(19) United States
(12) Patent Application Publication
(10) Pub. No.: US 2025/0188110 A1
(43) Pub. Date: Jun. 12, 2025

(54) SPECIFIC SMALL MOLECULE INHIBITORS THAT BLOCK KMT9 METHYLTRANSFERASE ACTIVITY AND FUNCTION

(71) Applicant: ALBERT-LUDWIGS-UNIVERSITÄT FREIBURG, Freiburg (DE)

(72) Inventors: Roland Schüle, Weisweil (DE); Sheng Wang, Freiburg im Breisgau (DE); Eric Metzger, Algolsheim (FR); Manfred Jung, Gundelfingen (DE); Sebastian Klein, Stein AG (CH)

(73) Assignee: ALBERT-LUDWIGS-UNIVERSITÄT FREIBURG, Freiburg (DE)

(21) Appl. No.: 18/683,022

(22) PCT Filed: Aug. 12, 2022
(86) PCT No.: PCT/EP2022/072677

§ 371 Date: Feb. 12, 2024

(30) Foreign Application Priority Data
Aug. 13, 2021 (EP) 21191163.1

(51) Int. Cl. C07H 19/167; A61K 31/519; A61K 31/5377; A61K 31/55; A61K 31/7064; A61K 31/7076; A61P 35/00; C07D 487/04; C07D 487/16; C07D 519/00; C07H 19/14

(52) U.S. Cl. CPC C07H 19/167; A61K 31/519; A61K 31/5377; A61K 31/55; A61K 31/7064; A61K 31/7076; A61P 35/00; C07D 487/04; C07D 487/16; C07D 519/00; C07H 19/14

(57) ABSTRACT

The present invention relates to novel specific small molecule inhibitors that block KMT9 methyltransferase activity. In particular, the present invention is concerned with a compound of formula (I) wherein X¹, X², X³, X⁴, R¹, R², R³, R⁵, R⁶ and L are as defined herein. Further, the present invention is concerned with a pharmaceutical composition comprising a pharmaceutically effective amount of the compound of formula (I). The present invention also relates to a compound of formula (I) and a pharmaceutical composition comprising a compound of formula (I) for use in medicine. Yet further, the present invention is concerned with a compound of formula (I) and a pharmaceutical composition comprising a compound of formula (I) for use as inhibitor of KMT9. Finally, the present invention is concerned with a compound of formula (I), wherein X¹, X², X³, X⁴, R¹, R², R³, R⁵, R⁶ and L are as defined herein, for use in the treatment of cancer selected from the group as defined herein

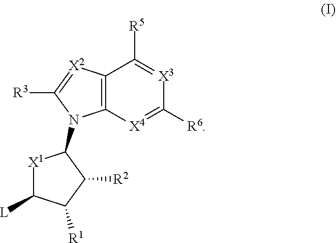

### FIELD OF THE INVENTION

The present invention relates to novel specific small molecule inhibitors that block KMT9 methyltransferase activity. In particular, the present invention is concerned with a compound of formula (I)

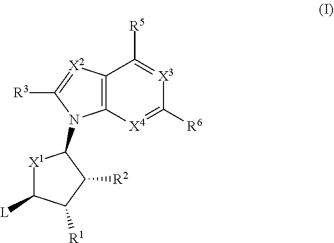

wherein X¹, X², X³, X⁴, R¹, R², R³, R⁵, R⁶ and L are as defined herein. Further, the present invention is concerned with a pharmaceutical composition comprising a pharmaceutically effective amount of the compound of formula (I). The present invention also relates to a compound of formula (I) and a pharmaceutical composition comprising a compound of formula (I) for use in medicine. Yet further, the present invention is concerned with a compound of formula (I) and a pharmaceutical composition comprising a compound of formula (I) for use as inhibitor of KMT9. Finally, the present invention is concerned with a compound of formula (I), wherein X¹, X², X³, X⁴, R¹, R², R³, R⁵, R⁶ and L are as defined herein, for use in the treatment of cancer selected from the group as defined herein.

### BACKGROUND OF THE INVENTION

Posttranslational modifications of histones such as methylation regulate chromatin structure and gene expression, and dysregulation of these mostly reversible modifications has been shown to have a central role in cancer onset and cancer progression (Strahl, B.D. & Allis, C.D. Nature 203, 41-45, doi: 10.1038/47412 (2000)).

Histone methyl transferases (HMT) possess high selectivity as regards the targeted histone lysine residue. Further, the pattern of methylation is specific for each HMT. There are two families of HMTs, namely the SET domain-containing HMTs (with the four subfamilies SET1 [a specific member here is EZH2], SET2, SUV39 and RIZ) and other HMTS, wherein e.g. DOT1L does not contain a SET domain but is a member of the seven-beta-strand family of histone methyltransferases. Further details in this respect as well as information on the effect of HMT-inhibition and specific inhibitors can be found in the review by Morera et al. Clinical Epigenetics, 8:57 (2016), doi: 10.1186/s13148-016-0223-4, 2016.

EZH2 and DOT1L have in particular been studied over the last years when it comes to their role in cancer. EZH2 is the catalytic component of the polycomb repressive complex 2 (PRc2), which performs three successive methyl transfer reactions arriving at H3K27me3. DOTIL is capable of catalyzing mono-, di-, and trimethylation of H3K79. While H3K79 is an activating mark when it comes to gene transcription, H3K27me3 is associated with gene silencing.

The inhibition of DOT1L is in particular implicated in the treatment of leukemias presenting a chromosomal translocation of the mixed-lineage leukemia (MLL) gene (chromosome 11q23), such as e.g., acute myeloid leukemias (AML), acute lymphoblastic leukemias (ALL) and the biphenotypic (mixed lineage) leukemias (MLL).
Recently, a further member of the seven-beta-strand family of histone methyltransferases was identified by Metzger et al., namely KMT9, a heterodimer comprised of KMT9alpha and KMT9beta (see Metzger et al., Nat. Struct. Mol. Biol., 2019 May 26 (5): 361). KMT9 writes the methylation mark on lysine 12 of histone H4 and H4K12 methylation has been shown to be implicated in prostate tumor cell proliferation.

There is of course an ongoing need for novel compounds that inhibit HMTs, in particular members of the seven-beta-strand family, preferably KMT9 as recent member of this family.

### OBJECTS AND SUMMARY OF THE INVENTION

It is therefore an object of the present invention to provide compounds, which inhibit HMTs, preferably members of the seven-beta-strand family, more preferably KMT9.

It is another object of the present invention to provide compounds, which are suitable for use as a medicament. It is another object of the present invention to provide compounds, which are suitable for use in the treatment of cancer linked to the inhibition of KMT9. In particular, it is an object to provide compounds, which are suitable for the treatment of cancer selected from the group consisting of prostate cancer, breast cancer, ovarian cancer, colon cancer, glioblastoma, lung cancer, neuroblastoma, osteosarcoma, liposarcoma, colorectal cancer, rectal adenocarcinoma, mesothelioma, endometrium adenocarcinoma, leukemia, erythroleukemia, medulloblastoma, astrocytoma, Ewing sarcoma, myelodysplastic syndrome (MDS), diffuse large

B-cell lymphoma, myelogenic leukemia, myeloid leukemia, acute monocytic leukemia, gallbladder carcinoma, cecum adenocarcinoma, gastric adenocarcinoma, stomach adenocarcinoma, renal cell carcinoma, bladder carcinoma, melanoma, cervical squamous cell carcinoma, pancreatic carcinoma, chondrosarcoma, duodenal adenocarcinoma, rhabdomyosarcoma, hepatocellular carcinoma and uterine adenocarcinoma.

The above objects can be achieved by the compounds of formula (I) as defined herein as well as pharmaceutical compositions comprising the same, and by the medical uses thereof.

The inventors of the present invention inter alia surprisingly found that the compounds of formula (I) as defined herein inhibit HMTs, in particular members of the seven-beta-strand family, preferably KMT9. Accordingly, the compounds of formula (I) can be used as a medicament, in particular for the treatment of cancer selected from the group consisting of prostate cancer, breast cancer, ovarian cancer, colon cancer, glioblastoma, lung cancer, neuroblastoma, osteosarcoma, liposarcoma, colorectal cancer, rectal adenocarcinoma, mesothelioma, endometrium adenocarcinoma, leukemia, erythroleukemia, medulloblastoma, astrocytoma, Ewing sarcoma, myelodysplastic syndrome (MDS), diffuse large B-cell lymphoma, myelogenic leukemia, myeloid leukemia, acute monocytic leukemia, gallbladder carcinoma, cecum adenocarcinoma, gastric adenocarcinoma, stomach adenocarcinoma, renal cell carcinoma, bladder carcinoma, melanoma, cervical squamous cell carcinoma, pancreatic carcinoma, chondrosarcoma, duodenal adenocarcinoma, rhabdomyosarcoma, hepatocellular carcinoma and uterine adenocarcinoma.

In a first aspect, the present invention therefore relates to a compound of formula (I)

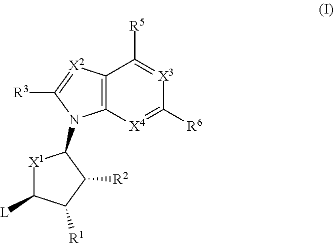

or a salt, stereoisomer, tautomer or N-oxide thereof,

wherein
X¹ is CH₂, N or O;
X² is CR⁴ or N;
X³ is CH or N;
X⁴ is CH, or N;
to 6-membered saturated, partially or fully unsaturated, or aromatic carbocyclyl, or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x};
R⁵ is H, C₁-C₄-alkyl, or NR^{a}R^{b}; or
R⁴ and R⁵ together with the atoms to which they are bonded form a 7- to 10-membered partially or fully unsaturated carbocyclyl or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x};
R⁶ is H, halogen, C₁-C₄-alkyl, NR^{a}R^{b}, or OR^{c};
wherein
L (i) is a 4- to 6-membered saturated heterocyclyl, wherein the aforementioned heterocyclic ring comprises one or more, same or different heteroatoms selected from O, N and S, and wherein said N-and/or S-atoms are independently oxidized or non-oxidized, or
   (ii) is selected from

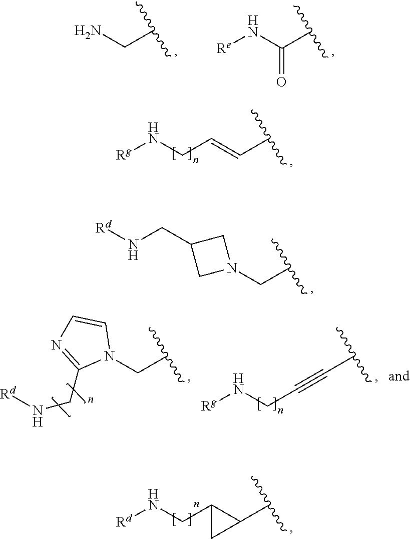

wherein
each substitutable carbon atom in the aforementioned groups is independently unsubstituted or substituted with one or more, same or different substituents selected from halogen, or two substituents together form C(═O);
wherein
R^{a}, R^{b} are independently of each other selected from H, C₁-C₄-alkyl and phenyl;
R^{c} is H, C₁-C₄-alkyl, NR^{a}R^{b}, NR^{a}R^{b}—C₁-C₄-alkyl, benzyl, or a 5- or 6-membered saturated heterocyclyl, wherein the aforementioned heterocyclic ring comprises one or more, same or different heteroatoms selected from O, N and S, and wherein said N- and/or S-atoms are independently oxidized or non-oxidized;
R^{d} is benzophenone-C₁-C₄-alkyl, phenoxybenzene-C₁-C₄-alkyl, N-methyl-diphenylamine-C₁-C₄-alkyl, or diphenylsulfide-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae

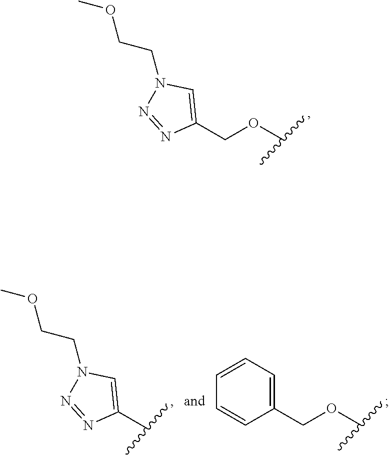

R^{e} is a 5- or 6-membered saturated heterocyclyl, wherein said heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x};
R^{g} is H, C(═O) R^{c}, phenyl-C₁-C₄-alkyl, benzophenone-C₁-C₄-alkyl, phenoxybenzene-C₁-C₄-alkyl, N-methyl-diphenylamine-C₁-C₄-alkyl, diphenylsulfide-C₁-C₄-alkyl, or naphthalene-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae

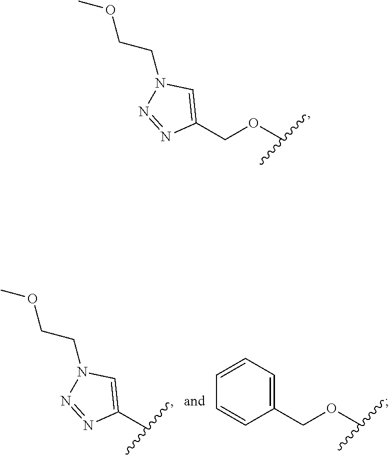

R^{x} is C₁-C₄-alkyl, C(═O) R^{c}, or phenyl-C₁-C₄-alkyl; and
n is 1, 2, 3 or 4;
with the proviso that if X² is N or CR⁴, wherein R⁴ is H, then R⁵ is not H or NH₂.

In a preferred embodiment, the present invention relates to a compound of formula (I), wherein
X¹ is CH₂ or O;
5 X² is CR⁴;
X³ is N; and
X⁴ is N;
with the proviso that if X² is CR⁴, wherein R⁴ is H, then R⁵ is not H or NH₂.

In another preferred embodiment, the present invention refers to a compound of formula (I), 10 wherein
R¹, R² are OH;
R³ is H;
R⁴ is H, halogen, C₁-C₄-alkyl or
   a 5-membered aromatic heterocyclyl, wherein the aforementioned heterocyclic ring comprises one or more, same or different heteroatoms selected from O, N and S, wherein said N-and/or S-atoms are independently oxidized or non-oxidized;
R⁵ is NR^{a}R^{b}; or
R⁴ and R⁵ together with the atoms to which they are bonded form a 7- to 10-membered partially or fully unsaturated carbocyclyl or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x};
R⁶ is H;
with the proviso that if X² is CR⁴, wherein R⁴ is H, then R⁵ is not NH₂.

In another preferred embodiment, the present invention relates to a compound of formula (I), wherein
L (i) is a 4- to 6-membered saturated heterocyclyl, wherein the aforementioned heterocyclic ring comprises one or more, same or different heteroatoms selected from O, N and S, and wherein said N-and/or S-atoms are independently oxidized or non-oxidized, or
(ii) is selected from

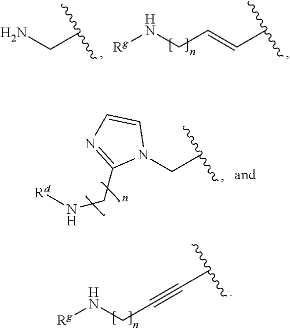

In a further aspect, the present invention relates to a pharmaceutical composition comprising a 35 pharmaceutically effective amount of the compound of formula (I) as defined herein and optionally a pharmaceutically acceptable carrier, diluent, or excipient.

In yet another aspect, the present invention relates to a compound of formula (I) as defined herein or a pharmaceutical composition comprising the same as defined herein for use in medicine.

In yet another aspect, the present invention relates to a compound of formula (I) as defined herein or a pharmaceutical composition comprising the same as defined herein for use in the treatment of cancer selected from the group consisting of prostate cancer, breast cancer, ovarian cancer, colon cancer, glioblastoma, lung cancer, neuroblastoma, osteosarcoma, liposarcoma, colorectal cancer, rectal adenocarcinoma, mesothelioma, endometrium adenocarcinoma, leukemia, erythroleukemia, medulloblastoma, astrocytoma, Ewing sarcoma, myelodysplastic syndrome (MDS), diffuse large B-cell lymphoma, myelogenic leukemia, myeloid leukemia, acute monocytic leukemia, gallbladder carcinoma, cecum adenocarcinoma, gastric adenocarcinoma, stomach adenocarcinoma, renal cell carcinoma, bladder carcinoma, melanoma, cervical squamous cell carcinoma, pancreatic carcinoma, chondrosarcoma, duodenal adenocarcinoma, rhabdomyosarcoma, hepatocellular carcinoma and uterine adenocarcinoma.

In one embodiment, the compound of formula (I) as defined herein or the pharmaceutical composition comprising the same as defined herein is for use in the treatment of cancer selected from the group consisting of prostate cancer, breast cancer, ovarian cancer, colon cancer, glioblastoma, lung cancer, neuroblastoma, colorectal cancer and bladder carcinoma.

In a second aspect, the present invention relates to a compound of formula (I)

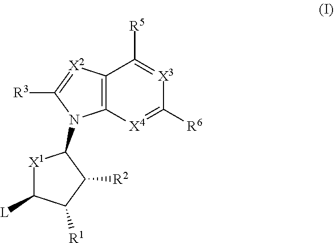

or a salt, stereoisomer, tautomer or N-oxide thereof,
wherein
X¹ is CH₂, N or O;
X² is CR⁴, or N;
X³ is CH, or N;
X⁴ is CH, or N;
R¹, R² are independently of each other selected from H, halogen and OH;
R³ is H, or C₁-C₄-alkyl;
R⁴ is H, CN, halogen, C₁-C₄-alkyl, C₂-C₄-alkenyl, phenyl, or a 3- to 6-membered saturated, partially or fully unsaturated, or aromatic carbocyclyl, or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more same or different substituents R^{x};
R⁵ is H, halogen, C₁-C₄-alkyl, C₁-C₄-haloalkyl, or NR^{a}R^{b}; or
R⁴ and R⁵ together with the atoms to which they are bonded form a 7- to 10-membered partially or fully unsaturated carbocyclyl or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x};
R⁶ is H, halogen, C₁-C₄-alkyl, NR^{a}R^{b}, or OR^{c};
wherein
L (i) is a 4- to 6-membered saturated, partially or fully unsaturated or aromatic heterocyclyl, or heterocyclyl-C₁-C₄-alkyl, wherein the aforementioned heterocyclic rings comprise one or more, same or different heteroatoms selected from O, N and S, and wherein said N-and/or S-atoms are independently oxidized or non-oxidized, and wherein each substitutable carbon or heteroatom in the aforementioned groups is independently unsubstituted or substituted with one or more, same or different substituents R^{Y}, or
   a 5- or 6-membered aromatic carbocyclyl, wherein the aforementioned carbocyclic ring is independently unsubstituted or substituted with one or more, same or different substituents R^{Y}; or
   a 7- to 10-membered aromatic carbobicyclyl or heterobicyclyl, wherein the aforementioned heterobicyclic ring comprises one or more, same or different heteroatoms selected from O, N and S, and wherein said N-and/or S-atoms are independently oxidized or non-oxidized, and wherein each substitutable carbon or heteroatom in the aforementioned groups is independently unsubstituted or substituted with one or more, same or different substituents R^{Y}; or
(ii) is selected from

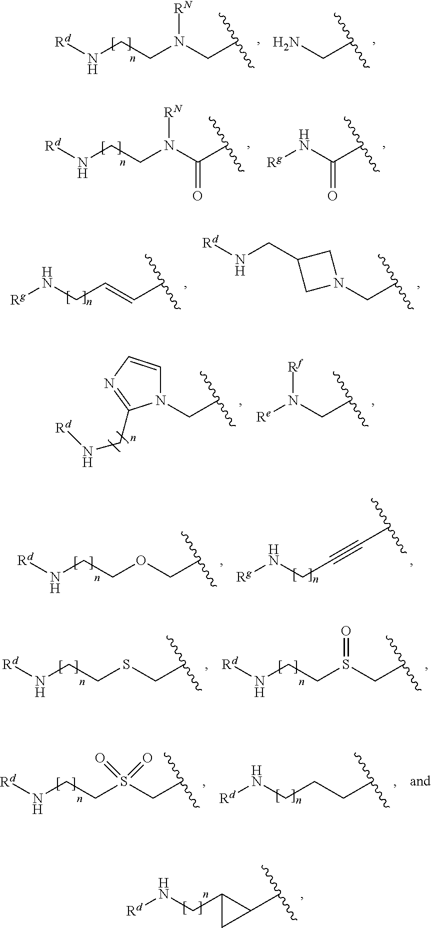

wherein
each substitutable carbon atom in the aforementioned groups is independently unsubstituted or substituted with one or more, same or different substituents selected from halogen, or two substituents together form C(═O);
R^{N} is H, C₁-C₄-alkyl, C₁-C₄-haloalkyl, NR^{a}R^{b}, cyclopropyl-C₁-C₄-alkyl, or a 5- or 6-membered saturated, partially or fully unsaturated or aromatic carbocyclyl, or heterocyclyl, wherein the aforementioned heterocyclic ring comprises one or more, same or different heteroatoms selected from O, N and S, wherein said N-and/or S-atoms are independently oxidized or non-oxidized and wherein each substitutable carbon or heteroatom in the aforementioned groups is independently unsubstituted or substituted with one or more, same or different substituents R^{x}; and wherein
R^{a}, R^{b} are independently of each other selected from H, C₁-C₄-alkyl, and phenyl;
R^{c} is H, C₁-C₄-alkyl, NR^{a}R^{b}, NR^{a}R^{b}—C₁-C₄-alkyl, benzyl, or a 5- or 6-membered saturated heterocyclyl, wherein the aforementioned heterocyclic ring comprises one or more, same or different heteroatoms selected from O, N and S, and wherein said N-and/or S-atoms are independently oxidized or non-oxidized;
R^{d} is H, C(═O)R^{c}, phenyl-C₁-C₄-alkyl, benzophenone-C₁-C₄-alkyl, phenoxybenzene-C₁-C₄-alkyl, N-methyl-diphenylamine-C₁-C₄-alkyl, diphenylsulfide-C₁-C₄-alkyl, or naphthalene-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae

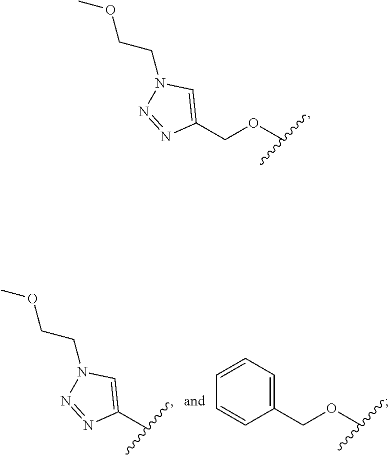

R^{e} is C₁-C₄-alkyl, C(═O) R^{c}, or a 5- or 6-membered saturated heterocyclyl, or heterocyclyl-C₁-C₄-alkyl, wherein said heterocyclic rings comprise one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x};
R^{f} is H, or C₁-C₄-alkyl; or
R^{e} and R^{f} together with the nitrogen atom to which they are bonded form a 7- to 10-membered saturated spiro-heterocyclyl, wherein the aforementioned heterocyclic ring comprises one or more, same or different heteroatoms selected from O, N and S, and wherein said N-and/or S-atoms are independently oxidized or non-oxidized;
R^{g} is H, C(═O) R^{c}, phenyl-C₁-C₄-alkyl, benzophenone-C₁-C₄-alkyl, phenoxybenzene-C₁-C₄-alkyl, N-methyl-diphenylamine-C₁-C₄-alkyl, diphenylsulfide-C₁-C₄-alkyl, or naphthalene-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae

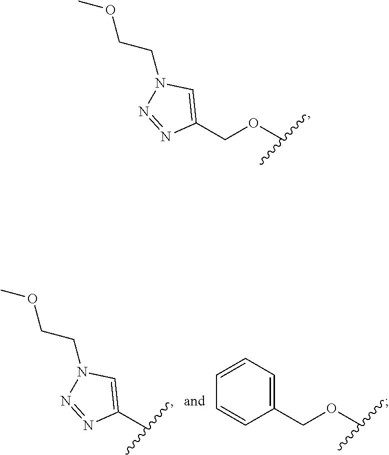

R^{h} is C₁-C₄-alkyl, or phenyl;
Rⁱ is C₁-C₄-alkyl, or phenyl;
R^{x} is halogen, C₁-C₄-alkyl, C(═O) R^{c}, S(═O) ¿R^{h}, ORⁱ, or phenyl-C₁-C₄-alkyl, or two R^{x} form ═O;
R^{Y} is halogen, NH₂, OH, C₁-C₄-alkyl, C₁-C₄-haloalkyl, C₂-C₆-alkenyl, C₁-C₄-alkoxy, C₁-C₄-haloalkoxy, cyclopropyl, cyclopropyl-C₁-C₄-alkyl, phenyl-C₁-C₄-alkyl, two R^{Y} form ═O, or a 4- to 6-membered saturated, partially or fully unsaturated or aromatic carbocyclyl, carbocyclyl-C₁-C₄-alkyl, heterocyclyl or heterocyclyl-C₁-C₄-alkyl, wherein the aforementioned heterocyclic rings comprise one or more, same or different heteroatoms selected from O, N and S, wherein said N-and/or S-atoms are independently oxidized or non-oxidized, and wherein each substitutable carbon in the aforementioned groups is independently unsubstituted or substituted with one or more, same or different substituents selected from NH₂, or two substituents form ═O; and
n is 1, 2, 3 or 4, for use in the treatment of prostate cancer.

In one embodiment of the present invention of said use, said prostate cancer is castration resistant prostate cancer.

In another embodiment of said use, in said compound of formula (I)
R^{x} is halogen, C₁-C₄-alkyl, C(═O) R^{c}, or phenyl-C₁-C₄-alkyl, or two R^{x} form ═O.

In another embodiment of said use, in said compound of formula (I)
X¹ is CH₂, or O;
X² is CR⁴;
X³ is N; and
X⁴ is N.

In another embodiment of said use, in said compound of formula (I)
R¹, R² are OH;
R³ is H; and
R⁶ is H, halogen, or C₁-C₄-alkyl.

In another embodiment of said use, in said compound of formula (I)
R⁴ is H, halogen, C₁-C₄-alkyl, C₂-C₄-alkenyl, phenyl, or a 3- to 6-membered fully unsaturated or aromatic heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more same or different substituents R^{x};
R⁵ is H, C₁-C₄-alkyl, or NR^{a}R^{b}; or
R⁴ and R⁵ together with the atoms to which they are bonded form a 7- to 10-membered partially or fully unsaturated carbocyclyl or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x}.

In another embodiment of said use, in said compound of formula (I)
L (i) is a 4- to 6-membered saturated, partially or fully unsaturated or aromatic heterocyclyl, or heterocyclyl-C₁-C₄-alkyl, wherein the aforementioned heterocyclic rings comprise one or more, same or different heteroatoms selected from O, N and S, and wherein said N-and/or S-atoms are independently oxidized or non-oxidized, and wherein each substitutable carbon or heteroatom in the aforementioned groups is independently unsubstituted or substituted with one or more, same or different substituents R^{Y}, or a 5- or 6-membered aromatic carbocyclyl, wherein the aforementioned carbocyclic ring is independently unsubstituted or substituted with one or more, same or different substituents R^{Y}; or
   (ii) is selected from

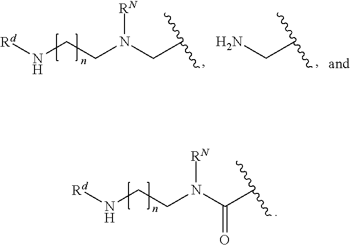

wherein
R^{N} is H, or a 5- or 6-membered saturated, partially or fully unsaturated or aromatic carbocyclyl, or heterocyclyl, wherein the aforementioned heterocyclic ring comprises one or more, same or different heteroatoms selected from O, N and S, wherein said N-and/or S-atoms are independently oxidized or non-oxidized and wherein each substitutable carbon or heteroatom in the aforementioned groups is independently unsubstituted or substituted with one or more, same or different substituents R^{x}; and
wherein
R^{d} is H, phenyl-C₁-C₄-alkyl, benzophenone-C₁-C₄-alkyl, phenoxybenzene-C₁-C₄-alkyl, or naphthalene-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae

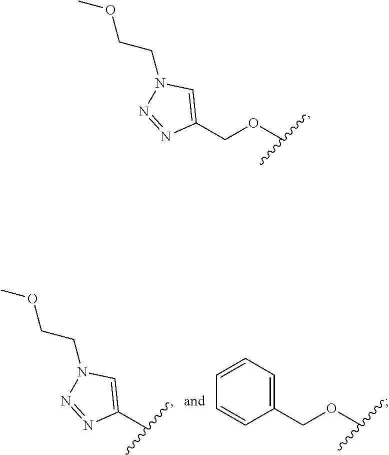

In another aspect, the present invention relates to a pharmaceutical composition comprising a pharmaceutically effective amount of the compound of formula (I) as defined in the above second aspect and optionally a pharmaceutically acceptable carrier, diluent, or excipient for use in the treatment of castration resistant prostate cancer.

In a third aspect, the present invention relates to a compound of formula (I)

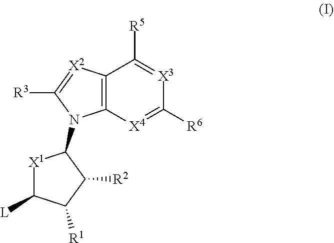

or a salt, stereoisomer, tautomer or N-oxide thereof,

wherein
X¹ is CH₂, or N;
X² is CR⁴ or N;
X³ is CH or N;
X⁴ is CH, or N;
R¹, R² are independently of each other selected from H, halogen and OH;
R³ is H, or C₁-C₄-alkyl;
R⁴ is H, CN, halogen, C₁-C₄-alkyl, C₂-C₄-alkenyl, phenyl, or a 3- to 6-membered saturated, partially or fully unsaturated, or aromatic carbocyclyl, or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more same or different substituents R^{x};
R⁵ is H, C₁-C₄-alkyl, C₁-C₄-haloalkyl, or NR^{a}R^{b}; or
R⁴ and R⁵ together with the atoms to which they are bonded form a 7- to 10-membered partially or fully unsaturated carbocyclyl or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x};
R⁶ is H, halogen, C₁-C₄-alkyl, NR^{a}R^{b}, or OR″;
wherein
L (i) is a 4- to 6-membered saturated, partially or fully unsaturated or aromatic heterocyclyl, wherein the aforementioned heterocyclic ring comprises one or more, same or different heteroatoms selected from O, N and S, and wherein said N-and/or S-atoms are independently oxidized or non-oxidized, and wherein each substitutable carbon or heteroatom in the aforementioned groups is independently unsubstituted or substituted with one or more, same or different substituents R^{Y}, or
   a 5- or 6-membered aromatic carbocyclyl, wherein the aforementioned carbocyclic ring is independently unsubstituted or substituted with one or more, same or different substituents R^{Y}; or
   a 7- to 10-membered aromatic carbobicyclyl or heterobicyclyl, wherein the aforementioned heterobicyclic ring comprises one or more, same or different heteroatoms selected from O, N and S, and wherein said N-and/or S-atoms are independently oxidized or non-oxidized, and wherein each substitutable carbon or heteroatom in the aforementioned groups is independently unsubstituted or substituted with one or more, same or different substituents R^{Y}; or
   (ii) is selected from
   wherein

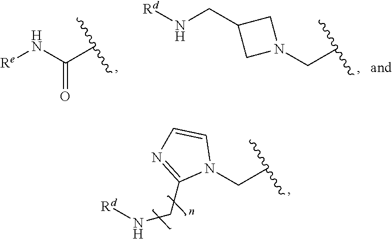

each substitutable carbon atom in the aforementioned groups is independently unsubstituted or substituted with one or more, same or different substituents selected from halogen, or two substituents together form C(═O);

wherein
R^{a}, R^{b} are independently of each other selected from H, C₁-C₄-alkyl and phenyl;
R^{c} is H, C₁-C₄-alkyl, NR^{a}R^{b}, NR^{a}R^{b}—C₁-C₄-alkyl, benzyl, or a 5- or 6-membered saturated heterocyclyl, wherein the aforementioned heterocyclic ring comprises one or more, same or different heteroatoms selected from O, N and S, and wherein said N-and/or S-atoms are independently oxidized or non-oxidized;
R^{d} is benzophenone-C₁-C₄-alkyl, phenoxybenzene-C₁-C₄-alkyl, N-methyl-diphenylamine-C₁-C₄-alkyl, or diphenylsulfide-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae

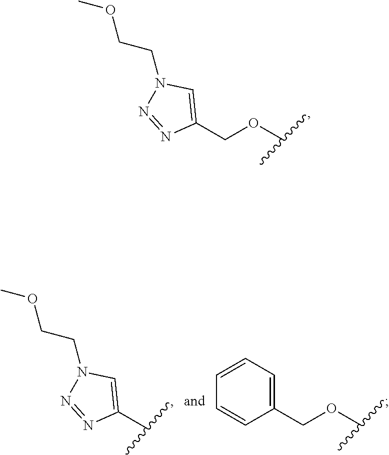

R^{e} is a 5- or 6-membered saturated heterocyclyl, wherein said heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x};
R^{g} is H, C(═O) R^{c}, phenyl-C₁-C₄-alkyl, benzophenone-C₁-C₄-alkyl, phenoxybenzene-C₁-C₄-alkyl, N-methyl-diphenylamine-C₁-C₄-alkyl, diphenylsulfide-C₁-C₄-alkyl, or naphthalene-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae

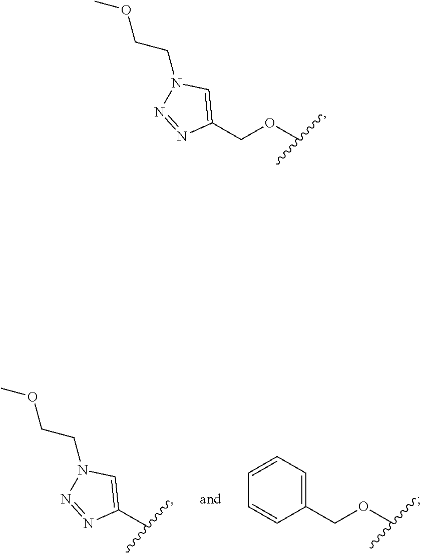

R^{h} is C₁-C₄-alkyl, or phenyl;
Rⁱ is C₁-C₄-alkyl, or phenyl;
R^{x} is C₁-C₄-alkyl, C(═O) R^{c}, S(═O) 2R^{h}, ORⁱ, or phenyl-C₁-C₄-alkyl;
R^{Y} is halogen, NH₂, OH, C₁-C₄-alkyl, C₁-C₄-haloalkyl, C₂-C₆-alkenyl, C₁-C₄-alkoxy, C₁-C₄-haloalkoxy, cyclopropyl, cyclopropyl-C₁-C₄-alkyl, phenyl-C₁-C₄-alkyl, two R^{Y} form ═O, or a 4- to 6-membered saturated, partially or fully unsaturated or aromatic carbocyclyl, carbocyclyl-C₁-C₄-alkyl, heterocyclyl or heterocyclyl-C₁-C₄-alkyl, wherein the aforementioned heterocyclic rings comprise one or more, same or different heteroatoms selected from O, N and S, wherein said N-and/or S-atoms are independently oxidized or non-oxidized, and wherein each substitutable carbon in the aforementioned groups is independently unsubstituted or substituted with one or more, same or different substituents selected from NH₂, or two substituents form ═O; and
n is 1, 2, 3 or 4;
with the proviso that if X² is N or CR⁴, wherein R⁴ is H, then R⁵ is not H or NH₂.

In a preferred embodiment, the present invention relates to a compound of formula (I), wherein
R^{x} is C₁-C₄-alkyl, C(═O) R^{c}, or phenyl-C₁-C₄-alkyl.

In another preferred embodiment, the present invention relates to a compound of formula (I), wherein
X¹ is CH₂;
X² is CR⁴.
X³ is N; and
X⁴ is N;
with the proviso that if X² is CR⁴, wherein R⁴ is H, then R⁵ is not H or NH₂.

In another preferred embodiment, the present invention relates to a compound of formula (I), wherein
R¹, R² are OH;
R³ is H;
R⁴ is H, halogen, C₁-C₄-alkyl or
   a 5-membered fully unsaturated or aromatic heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more same or different substituents R^{X}; R⁵ is H, or NR^{a}R^{b}; or R⁴ and R⁵ together with the atoms to which they are bonded form a 7- to 10-membered partially or fully unsaturated carbocyclyl or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x};
R⁶ is H, or halogen;
with the proviso that if X² is CR⁴, wherein R⁴ is H, then R⁵ is not H or NH₂.

In another preferred embodiment, the present invention relates to a compound of formula (I), wherein
L (i) is a 4- to 6-membered saturated, partially or fully unsaturated or aromatic heterocyclyl, wherein the aforementioned heterocyclic ring comprises one or more, same or different heteroatoms selected from O, N and S, and wherein said N-and/or S-atoms are independently oxidized or non-oxidized, and wherein each substitutable carbon or heteroatom in the aforementioned groups is independently unsubstituted or substituted with one or more, same or different substituents R^{Y}, or
   a 5- or 6-membered aromatic carbocyclyl, wherein the aforementioned carbocyclic ring is independently unsubstituted or substituted with one or more, same or different substituents R^{Y}.

In a further aspect, the present invention relates to a pharmaceutical composition comprising a pharmaceutically effective amount of the compound of formula (I) as defined above and optionally a pharmaceutically acceptable carrier, diluent, or excipient.

In yet another aspect, the present invention relates to a compound of formula (I) as defined above or a pharmaceutical composition comprising the same as defined above for use in medicine.

In yet another aspect, the present invention relates to a compound of formula (I) as defined above or a pharmaceutical composition comprising the same as defined above for use in the treatment of cancer selected from the group consisting of prostate cancer, breast cancer, ovarian cancer, colon cancer, glioblastoma, lung cancer, neuroblastoma, osteosarcoma, liposarcoma, colorectal cancer, rectal adenocarcinoma, mesothelioma, endometrium adenocarcinoma, leukemia, erythroleukemia, medulloblastoma, astrocytoma, Ewing sarcoma, myelodysplastic syndrome (MDS), diffuse large B-cell lymphoma, myelogenic leukemia, myeloid leukemia, acute monocytic leukemia, gallbladder carcinoma, cecum adenocarcinoma, gastric adenocarcinoma, stomach adenocarcinoma, renal cell carcinoma, bladder carcinoma, melanoma, cervical squamous cell carcinoma, pancreatic carcinoma, chondrosarcoma, duodenal adenocarcinoma, rhabdomyosarcoma, hepatocellular carcinoma and uterine adenocarcinoma.

In one embodiment, the compound of formula (I) as defined above or the pharmaceutical composition comprising the same as defined above is for use in the treatment of cancer selected from the group consisting of prostate cancer, breast cancer, ovarian cancer, colon cancer, glioblastoma, lung cancer, neuroblastoma, colorectal cancer and bladder carcinoma.

### DETAILED DESCRIPTION

In the following, preferred embodiments of the substituents of the compounds of formula (I) according to the first aspect of the present invention are described in further detail. It is to be understood that each preferred embodiment is relevant on its own as well as in combination with other preferred embodiments. Furthermore, it is to be understood that the preferences in each case also apply to the salts, stereoisomers, tautomers, and N-oxides of the compounds according to formula (I) of the invention.

As indicated above, in a first aspect the present invention relates to a compound of formula (I)

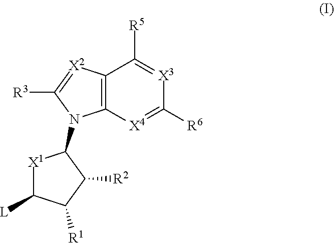

or a salt, stereoisomer, tautomer or N-oxide thereof,

wherein
X¹ is CH₂, N or O;
X² is CR⁴ or N;
X³ is CH or N; and
X⁴ is CH, or N.

In a preferred embodiment, the present invention relates to a compound of formula (I)

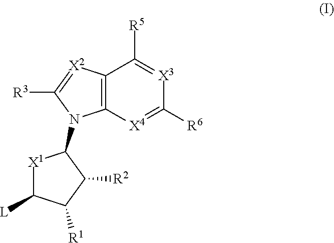

wherein the following substituent meanings are preferred for X¹, X², x3 and X⁴:
X¹ is CH₂ or O;
X² is CR⁴;
X³ is N; and
X⁴ is N.

Thus, in a more preferred embodiment, the compound of formula (I) is a compound of formula (Ia) or formula (Ib)

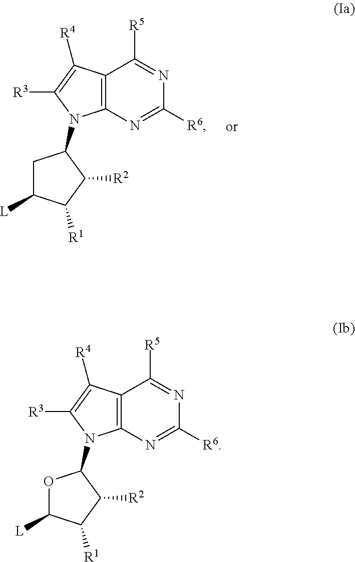

In connection with the above preferred and more preferred embodiments it is to be understood that R¹, R², R³, R⁴, R⁵, R⁶ and L are as defined above with regard to the compound of formula (I) of the first aspect of the present invention.

Furthermore, it is to be understood with regard to the above preferred and more preferred embodiments that if X² is N or CR⁴, wherein R⁴ is H, then R⁵ is not H or NH₂, preferably it is to be understood that if X² is CR⁴, wherein R⁴ is H, then R⁵ is not H or NH₂.

In a preferred embodiment of the present invention with regard to the compounds of formula (I), preferably with regard to the compounds of formula (Ia) and (Ib) the following substituent meanings with regard to R¹, R², R³, R⁴, R⁵ and R⁶ are preferred:
R¹, R² are independently of each other selected from H, halogen and OH;
R³ is H, or C₁-C₄-alkyl;
R⁴ is H, CN, halogen, C₁-C₄-alkyl, C₂-C₄-alkenyl, phenyl, or a 3- to 6-membered saturated, partially or fully unsaturated, or aromatic carbocyclyl, or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more same or different substituents R^{x};
R⁵ is H, C₁-C₄-alkyl, or NR^{a}R^{b}; or
R⁴ and R⁵ together with the atoms to which they are bonded form a 7- to 10-membered partially or fully unsaturated carbocyclyl or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x}; and
R⁶ is H, halogen, C₁-C₄-alkyl, NR^{a}R^{b}, or OR^{c}.

In a more preferred embodiment of the present invention with regard to the compounds of formula (I), preferably with regard to the compounds of formula (Ia) and (Ib) the following substituent meanings with regard to R¹, R², R³, R⁴, R⁵ and R⁶ are preferred:
R¹, R² are independently of each other selected from halogen and OH;
R³ is H;
R⁴ is H, halogen, C₁-C₄-alkyl or
   a 5-membered aromatic heterocyclyl, wherein the aforementioned heterocyclic ring comprises one or more, same or different heteroatoms selected from O, N and S, wherein said N-and/or S-atoms are independently oxidized or non-oxidized;
R⁵ is NR^{a}R^{b}; or
R⁴ and R⁵ together with the atoms to which they are bonded form a 7- to 10-membered partially or fully unsaturated carbocyclyl or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x}; and
R⁶ is H.

In another more preferred embodiment of the present invention with regard to the compounds of formula (I), preferably with regard to the compounds of formula (Ia) and (Ib) the following substituent meanings with regard to R¹, R², R³, R⁴, R⁵ and R⁶ are preferred:
R¹, R² are independently of each other selected from F and OH;
R³ is H;
R⁴ is H, halogen, C₁-C₄-alkyl or
   a 5-membered aromatic heterocyclyl, wherein the aforementioned heterocyclic ring comprises one or more, same or different heteroatoms selected from O, N and S, wherein said N-and/or S-atoms are independently oxidized or non-oxidized;
R⁵ is NR^{a}R^{b}; or
R⁴ and R⁵ together with the atoms to which they are bonded form a 7- to 10-membered partially or fully unsaturated carbocyclyl or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x}; and
R⁶ is H.

In a particularly preferred embodiment of the present invention with regard to the compounds of formula (I), preferably with regard to the compounds of formula (Ia) and (Ib) the following substituent meanings with regard to R¹, R², R³, R⁴, R⁵ and R⁶ are preferred:
R¹, R² are OH;
R³ is H;
R⁴ is H, halogen, C₁-C₄-alkyl or
   a 5-membered aromatic heterocyclyl, wherein the aforementioned heterocyclic ring comprises one or more, same or different heteroatoms selected from O, N and S, wherein said N-and/or S-atoms are independently oxidized or non-oxidized;
R⁵ is NR^{a}R^{b}; or
R⁴ and R⁵ together with the atoms to which they are bonded form a 7- to 10-membered partially or fully unsaturated carbocyclyl or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x}; and R⁶ is H.

Thus, in an even more preferred embodiment of the present invention, the compound of formula (I), preferably the compound of formula (Ia) or (Ib), is a compound according to formula (I.1a), or (I.1b)

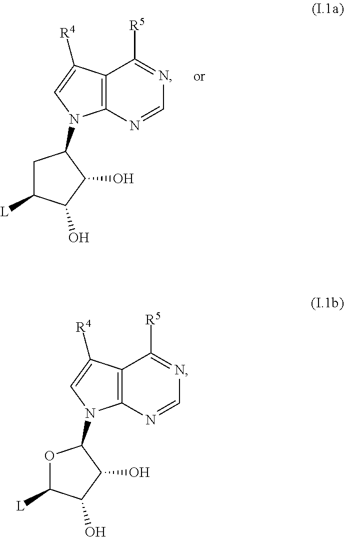

wherein
R⁴ is H, halogen, C₁-C₄-alkyl or
   a 5-membered aromatic heterocyclyl, wherein the aforementioned heterocyclic ring comprises one or more, same or different heteroatoms selected from O, N and S, wherein said N-and/or S-atoms are independently oxidized or non-oxidized;
R⁵ is NR^{a}R^{b}; or
R⁴ and R⁵ together with the atoms to which they are bonded form a 7- to 10-membered partially or fully unsaturated carbocyclyl or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x}.

In connection with the above preferred embodiments it is to be understood that R^{a}, R^{b}, R^{x} and L are as defined above with regard to the compounds of formula (I) of the first aspect of the present invention.

Preferably, R⁴ and R⁵ have the following substituent meanings with regard to the compounds of formula (I), preferably with regard to the compounds of formula (Ia) and (Ib), and more preferably with regard to the compounds of formula (I.1a), and (I.1b)
R⁴ is H, I, CH₃ or thiazolyl;
R⁵ is NH₂, or NHCH₃, or
R⁴ and R⁵ together with the atoms to which they are bonded form a 7-membered partially unsaturated heterocyclyl, wherein the aforementioned heterocyclic ring comprises one or more, same or different heteroatoms selected from O, N and S, wherein said N-and/or S-atoms are independently oxidized or non-oxidized.

In connection with the above preferred embodiments it is to be understood that if X² is CR⁴, wherein R⁴ is H, then R⁵ is not NH₂. Furthermore, it is to be understood that L is as defined above with regard to the compounds of formula (I) of the first aspect of the present invention.

In a preferred embodiment of the present invention with regard to the compounds of formula (I), preferably with regard to the compounds of formula (Ia) and (Ib), and more preferably with regard to the compounds of formula (I.1a) and (I.1b) the following substituent meanings with regard to L are preferred:
L (i) is a 4- to 6-membered saturated heterocyclyl, wherein the aforementioned heterocyclic ring comprises one or more, same or different heteroatoms selected from O, N and S, and wherein said N-and/or S-atoms are independently oxidized or non-oxidized, or
   (ii) is selected from

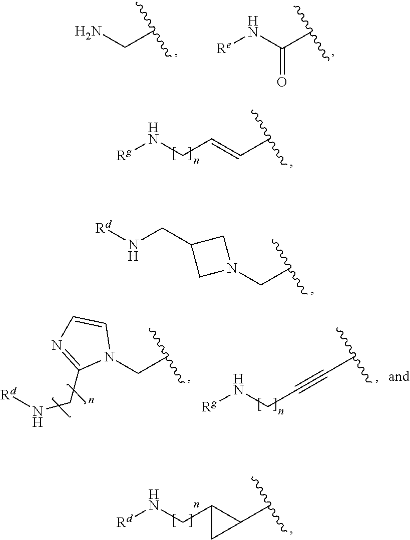

wherein
each substitutable carbon atom in the aforementioned groups is independently unsubstituted or substituted with one or more, same or different substituents selected from halogen, or two substituents together form C(═O).

In connection with the above definitions for L it is to be understood that the curled line in the structural formula indicates the connection to the remainder of the molecule

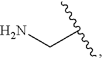 [SMILES: *CN]

it is to be understood that the group may be also present in the form of its salt. Preferably, the group of L being

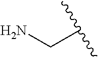 [SMILES: *CN]

may be present in the form of the trifluoroacetic acid salt, preferably the 2, 2, 2-being trifluoroacetic acid salt. Further, it is to be understood that R^{d}, R^{e}, R^{g} and n are as defined above with regard to the compounds of formula (I) of the first aspect of the present invention or as defined further below.

In a more preferred embodiment of the present invention with regard to the compounds of formula (I), preferably with regard to the compounds of formula (Ia) and (Ib), and more preferably with regard to the compounds of formula (I.1a) and (I.1b) the following substituent meanings with regard to L are preferred:

L (i) is a 4- to 6-membered saturated heterocyclyl, wherein the aforementioned heterocyclic ring comprises one or more, same or different heteroatoms selected from O, N and S, and wherein said N-and/or S-atoms are independently oxidized or non-oxidized, or
(ii) is selected from

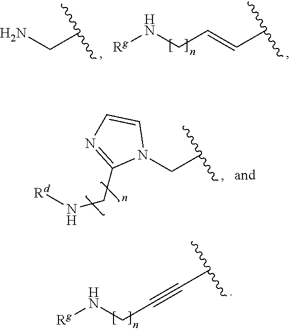

In connection with the above preferred embodiments it is to be understood that
R^{d} is benzophenone-C₁-C₄-alkyl, phenoxybenzene-C₁-C₄-alkyl, N-methyl-diphenylamine-C₁-C₄-alkyl, or diphenylsulfide-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae

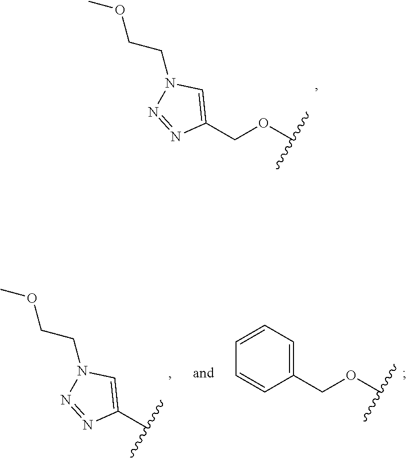

and
R^{g} is H, C(═O) R^{c}, phenyl-C₁-C₄-alkyl, benzophenone-C₁-C₄-alkyl, phenoxybenzene-C₁-C₄-alkyl, N-methyl-diphenylamine-C₁-C₄-alkyl, diphenylsulfide-C₁-C₄-alkyl, or naphthalene-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae

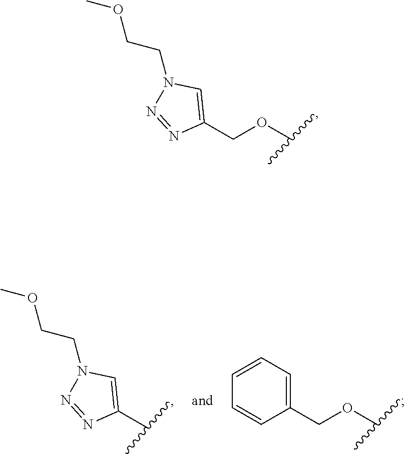

preferably
R^{d} is benzophenone-C₁-C₄-alkyl, preferably benzophenone-C₂-alkyl; and
R^{g} is phenyl-C₁-C₄-alkyl, preferably phenyl-C₂-alkyl.

Further, in connection with the above preferred embodiments it is to be understood that
R^{d} is phenoxybenzene-C₁-C₄-alkyl, preferably phenoxybenzene-C₂-alkyl; and
R⁹ is phenyl-C₁-C₄-alkyl, preferably phenyl-C₂-alkyl.

Furthermore, in connection with regard to the group of L being (ii)

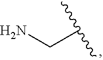 [SMILES: *CN]

it is to be understood that the group may be also present in the form of its salt. Preferably, the group of L being

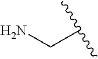 [SMILES: *CN]

may be present in the form of the trifluoroacetic acid salt, preferably the 2, 2, 2-trifluoroacetic acid salt.

Furthermore, in connection with the above preferred embodiments it is to be understood that n is 1, 2, 3 or 4.

Preferably, it is to be understood that n is 1, 2 or 3.

In connection with the above definitions for L, R^{d} and R^{g} it is to be understood that the curled line in the structural formula indicates the connection to the remainder of the molecule.

Thus, in a particularly preferred embodiment of the present invention with regard to the compounds of formula (I), preferably with regard to the compounds of formula (Ia) and (Ib), and more preferably with regard to the compounds of formula (I.1a) and (I.1b) the following substituent meanings with regard to L are preferred:
L (i) is a 6-membered saturated heterocyclyl, wherein the aforementioned heterocyclic ring comprises one or more, same or different heteroatoms selected from O, N and S, and wherein said N-and/or S-atoms are independently oxidized or non-oxidized,
preferably
L (i) is piperidinyl; or
L (ii) is

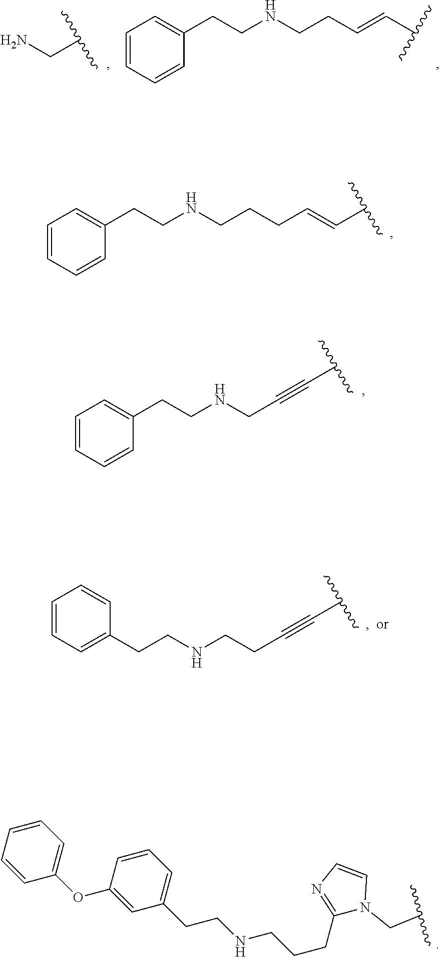

In connection with regard to the group of L being

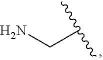 [SMILES: *CN]

it is to be understood that the group may be also present in the form of its salt. Preferably, the group of L being

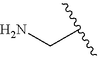 [SMILES: *CN]

may be present in the form of the trifluoroacetic acid salt, preferably the 2, 2, 2-trifluoroacetic acid salt.

Further, in connection with the above definitions for L it is to be understood that the curled line in the structural formula indicates the connection to the remainder of the molecule.

Thus, in another particularly preferred embodiment, the present invention relates to a compound of formula (I.1b)

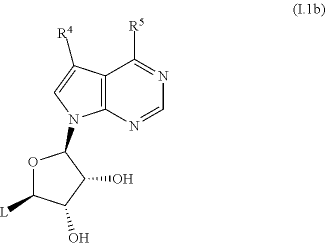

wherein
R⁴ is CH₃;

L is

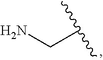 [SMILES: *CN]

wherein

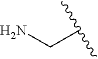 [SMILES: *CN]

is present in the form of the trifluoroacetic acid salt, preferably the 2, 2, 2-trifluoroacetic acid salt.

In another particularly preferred embodiment, the present invention relates to a compound of formula (I.1b)

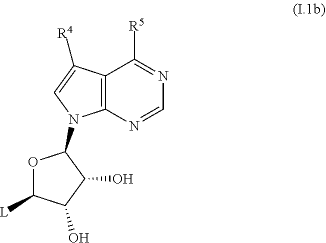

wherein
R⁴ is H;
R⁵ is NHCH₃; and
L is

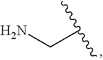 [SMILES: *CN]

wherein

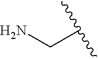 [SMILES: *CN]

is present in the form of the trifluoroacetic acid salt, wherein preferably the 2, 2, 2-trifluoroacetic acid salt.

In another particularly preferred embodiment, the present invention relates to a compound of formula (I.1b)

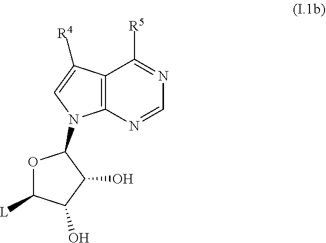

wherein
R⁴ and R⁵ together with the atoms to which they are bonded form a 7-membered partially unsaturated heterocyclyl, wherein the aforementioned heterocyclic ring comprises one nitrogen atom; and
L is

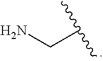 [SMILES: *CN]

In another particularly preferred embodiment, the present invention relates to a compound of formula (I.1a)

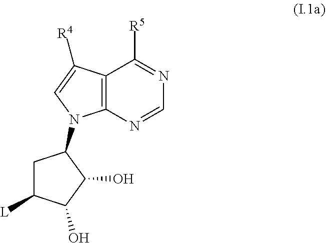

wherein
R⁴ is H;
R⁵ is NHCH₃; and
L is

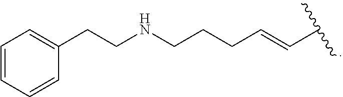 [SMILES: */C=C/CCCNCCc1ccccc1]

In another particularly preferred embodiment, the present invention relates to a compound of formula (I.1a)

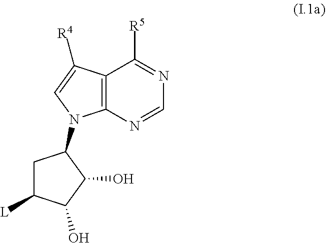

wherein
R⁴ is H;
R⁵ is NHCH₃; and
L is

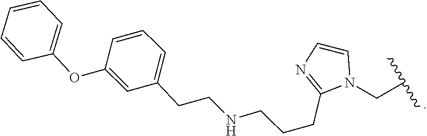 [SMILES: *Cn1ccnc1CCCNCCc1cccc(Oc2ccccc2)c1]

In another particularly preferred embodiment, the present invention relates to a compound of formula (I.1a)

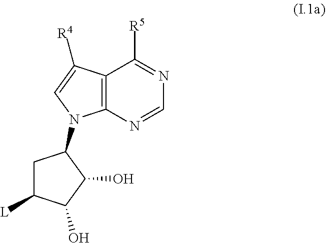

wherein
R⁴ is H;
R⁵ is NHCH₃; and
L is

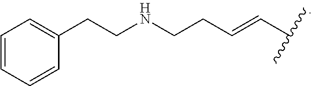 [SMILES: */C=C/CCNCCc1ccccc1]

In another particularly preferred embodiment, the present invention relates to a compound of formula (I.1a)

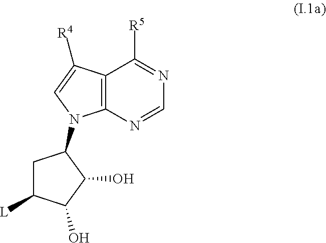

wherein
R⁴ is H;
R⁵ is NHCH₃; and
L is

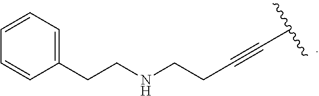 [SMILES: *C#CCCNCCc1ccccc1]

In another particularly preferred embodiment, the present invention relates to a compound of formula (I.1a)

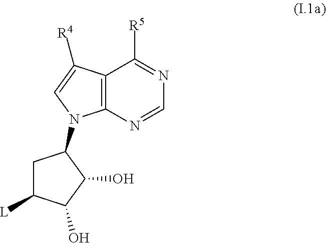

wherein
R⁴ is H;
R⁵ is NHCH₃; and
L is

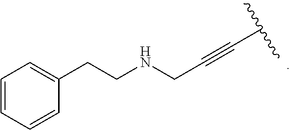 [SMILES: *C#CCNCCc1ccccc1]

In another particularly preferred embodiment, the present invention relates to a compound of formula (I.1a)

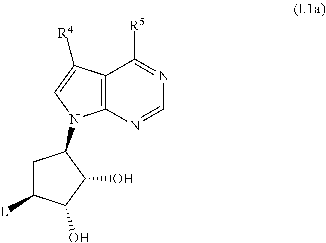

wherein
R⁴ is thiazolyl;
R⁵ is NH₂; and
L is piperidinyl.

Accordingly, in an even more particularly preferred embodiment of the present invention, the compound of formula (I) is selected from the group consisting of (1R,2S,3R,5R)-3-(4-(methylamino)-7H-pyrrolo[2,3-d]pyrimidin-7-yl)-5-(3-(phenethylamino)prop-1-yn-1-yl)cyclopentane-1,2-diol; (1R,2S,3R,5R)-3-(4-(methylamino)-7H-pyrrolo[2,3-d]pyrimidin-7-yl)-5-(4-(phenethylamino) but-1-yn-1-yl) cyclopentane-1,2-diol; (1R,2S,3R,5R)-3-(4-(methylamino)-7H-pyrrolo[2,3-d]pyrimidin-7-yl)-5-((E)-4-(phenethylamino) but-1-en-1-yl) cyclopentane-1,2-diol; (1R,2S,3R,5R)-3-(4-(methylamino)-7H-pyrrolo[2,3-d]pyrimidin-7-yl)-5-((2-(3-((3-phenoxyphenethyl) amino)propyl)-1H-imidazol-1-yl)methyl) cyclopentane-1,2-diol; (1R,2S,3R,5R)-3-(4-(methylamino)-7H-pyrrolo[2,3-d]pyrimidin-7-yl)-5-((E)-5-(phenethylamino) pent-1-en-1-yl) cyclopentane-1,2-diol; (2R,3S,4R,5R)-2-(aminomethyl)-5-(6,7,8,9-tetrahydro-2H-2,3,5,6-tetraazabenzo[cd]azulen-2-yl) tetrahydrofuran-3,4-diol; ((2R,3S,4R,5R)-3,4-dihydroxy-5-(4-(methylamino)-7H-pyrrolo[2,3-d]pyrimidin-7-yl) tetrahydrofuran-2-yl) methanaminium 2,2,2-trifluoroacetate; ((2R,3S,4R,5R)-3,4-dihydroxy-5-(5-methyl-4-(methylamino)-7H-pyrrolo[2,3-d]pyrimidin-7-yl) tetrahydrofuran-2-yl) methanaminium 2,2,2-trifluoroacetate; and (1R,2S,3R,5R)-3-(4-amino-5-(thiazol-2-yl)-7H-pyrrolo[2,3-d]pyrimidin-7-yl)-5-(piperidin-4-yl) cyclopentane-1,2-diol.

In a second aspect, the present invention relates to a compound of formula (I)

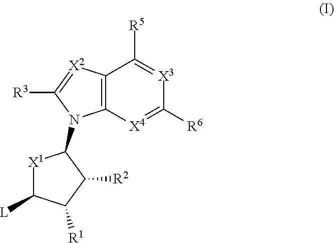

or a salt, stereoisomer, tautomer or N-oxide thereof,
wherein
X¹ is CH₂, N or O;
X² is CR⁴, or N;
X³ is CH, or N;
X⁴ is CH, or N;
R¹, R² are independently of each other selected from H, halogen and OH;
R³ is H, or C₁-C₄-alkyl;
R⁴ is H, CN, halogen, C₁-C₄-alkyl, C₂-C₄-alkenyl, phenyl, or a 3- to 6-membered saturated, partially or fully unsaturated, or aromatic carbocyclyl, or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more same or different substituents R^{x};
R⁵ is H, halogen, C₁-C₄-alkyl, C₁-C₄-haloalkyl, or NR^{a}R^{b}; or
R⁴ and R⁵ together with the atoms to which they are bonded form a 7- to 10-membered partially or fully unsaturated carbocyclyl or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x};
R⁶ is H, halogen, C₁-C₄-alkyl, NR^{a}R^{b}, or OR^{c};
wherein
L (i) is a 4- to 6-membered saturated, partially or fully unsaturated or aromatic heterocyclyl, or heterocyclyl-C₁-C₄-alkyl, wherein the aforementioned heterocyclic rings comprise one or more, same or different heteroatoms selected from O, N and S, and wherein said N-and/or S-atoms are independently oxidized or non-oxidized, and wherein each substitutable carbon or heteroatom in the aforementioned groups is independently unsubstituted or substituted with one or more, same or different substituents R^{Y}, or
   a 5- or 6-membered aromatic carbocyclyl, wherein the aforementioned carbocyclic ring is independently unsubstituted or substituted with one or more, same or different substituents R^{Y}; or
   a 7- to 10-membered aromatic carbobicyclyl or heterobicyclyl, wherein the aforementioned heterobicyclic ring comprises one or more, same or different heteroatoms selected from O, N and S, and wherein said N-and/or S-atoms are independently oxidized or non-oxidized, and wherein each substitutable carbon or heteroatom in the aforementioned groups is independently unsubstituted or substituted with one or more, same or different substituents R^{Y}; or
   (ii) is selected from

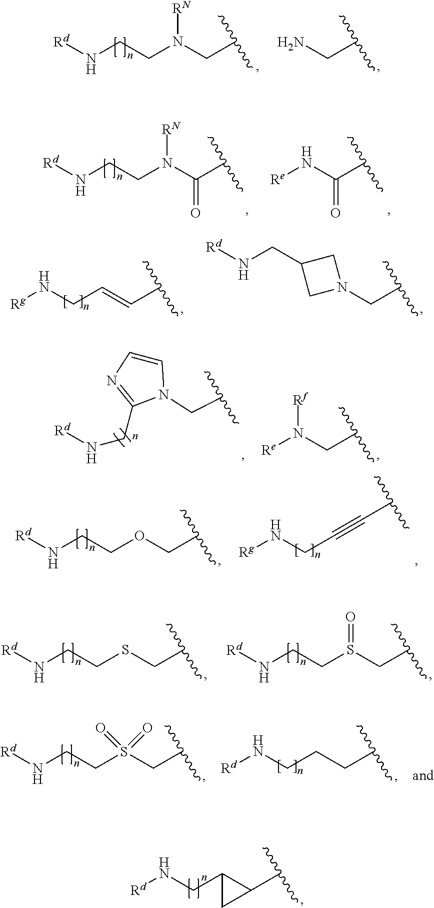

wherein
each substitutable carbon atom in the aforementioned groups is independently unsubstituted or substituted with one or more, same or different substituents selected from halogen, or two substituents together form C(═O);
R^{N} is H, C₁-C₄-alkyl, C₁-C₄-haloalkyl, NR^{a}R^{b}, cyclopropyl-C₁-C₄-alkyl, or a 5- or 6-membered saturated, partially or fully unsaturated or aromatic carbocyclyl, or heterocyclyl, wherein the aforementioned heterocyclic ring comprises one or more, same or different heteroatoms selected from O, N and S, wherein said N-and/or S-atoms are independently oxidized or non-oxidized and wherein each substitutable carbon or heteroatom in the aforementioned groups is independently unsubstituted or substituted with one or more, same or different substituents R^{x}; and wherein
R^{a}, R^{b} are independently of each other selected from H, C₁-C₄-alkyl, and phenyl;
R^{c} is H, C₁-C₄-alkyl, NR^{a}R^{b}, NR^{a}R^{b}—C₁-C₄-alkyl, benzyl, or a 5- or 6-membered saturated heterocyclyl, wherein the aforementioned heterocyclic ring comprises one or more, same or different heteroatoms selected from O, N and S, and wherein said N-and/or S-atoms are independently oxidized or non-oxidized;
R^{d} is H, C(═O) R^{c}, phenyl-C₁-C₄-alkyl, benzophenone-C₁-C₄-alkyl, phenoxybenzene-C₁-C₄-alkyl, N-methyl-diphenylamine-C₁-C₄-alkyl, diphenylsulfide-C₁-C₄-alkyl, or naphthalene-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae

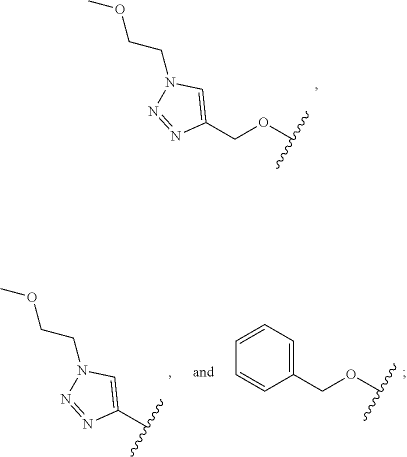

R^{e} is C₁-C₄-alkyl, C(═O) R^{c}, or a 5- or 6-membered saturated heterocyclyl, or heterocyclyl-C₁-C₄-alkyl, wherein said heterocyclic rings comprise one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x};
R^{f} is H, or C₁-C₄-alkyl; or
R^{e} and R^{f} together with the nitrogen atom to which they are bonded form a 7- to 10-membered saturated spiro-heterocyclyl, wherein the aforementioned heterocyclic ring comprises one or more, same or different heteroatoms selected from O, N and S, and wherein said N-and/or S-atoms are independently oxidized or non-oxidized;
R^{g} is H, C(═O) R^{c}, phenyl-C₁-C₄-alkyl, benzophenone-C₁-C₄-alkyl, phenoxybenzene-C₁-C₄-alkyl, N-methyl-diphenylamine-C₁-C₄-alkyl, diphenylsulfide-C₁-C₄-alkyl, or naphthalene-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae

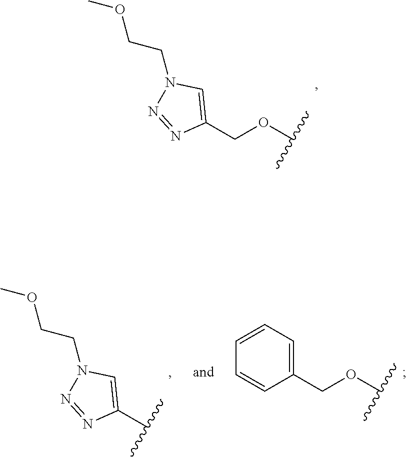

R^{h} is C₁-C₄-alkyl, or phenyl;
Rⁱ is C₁-C₄-alkyl, or phenyl;
R^{x} is halogen, C₁-C₄-alkyl, C(═O) R^{c}, S(═O) ¿R^{h}, ORⁱ, or phenyl-C₁-C₄-alkyl, or two R^{x} form ═O;
R^{Y} is halogen, NH₂, OH, C₁-C₄-alkyl, C₁-C₄-haloalkyl, C₂-C₆-alkenyl, C₁-C₄-alkoxy, C₁-C₄-haloalkoxy, cyclopropyl, cyclopropyl-C₁-C₄-alkyl, phenyl-C₁-C₄-alkyl, two R^{Y} form ═O, or a 4- to 6-membered saturated, partially or fully unsaturated or aromatic carbocyclyl, carbocyclyl-C₁-C₄-alkyl, heterocyclyl or heterocyclyl-C₁-C₄-alkyl, wherein the aforementioned heterocyclic rings comprise one or more, same or different heteroatoms selected from O, N and S, wherein said N-and/or S-atoms are independently oxidized or non-oxidized, and wherein each substitutable carbon in the aforementioned groups is independently unsubstituted or substituted with one or more, same or different substituents selected from NH₂, or two substituents form ═O; and
n is 1, 2, 3 or 4,
for use in the treatment of prostate cancer.

In a further embodiment of the second aspect, the present invention relates to a compound of formula (I)

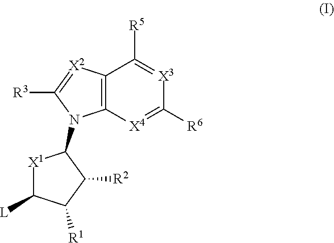

or a salt, stereoisomer, tautomer or N-oxide thereof,
wherein
X¹ is CH₂, N or O;
X² is CR⁴, or N;
X³ is CH, or N;
X⁴ is CH, or N;
R¹, R² are independently of each other selected from H, halogen and OH;
R³ is H, or C₁-C₄-alkyl;
R⁴ is H, CN, halogen, C₁-C₄-alkyl, C₂-C₄-alkenyl, phenyl, or a 3- to 6-membered saturated, partially or fully unsaturated, or aromatic carbocyclyl, or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more same or different substituents R^{x};
R⁵ is H, halogen, C₁-C₄-alkyl, or NR^{a}R^{b}; or
R⁴ and R⁵ together with the atoms to which they are bonded form a 7- to 10-membered partially or fully unsaturated carbocyclyl or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x};
R⁶ is H, halogen, C₁-C₄-alkyl, NR^{a}R^{b}, or OR”;
wherein
L (i) is a 4- to 6-membered saturated heterocyclyl, or heterocyclyl-C₁-C₄-alkyl, wherein the aforementioned heterocyclic rings comprise one or more, same or different heteroatoms selected from O, N and S, and wherein said N-and/or S-atoms are independently oxidized or non-oxidized, or
   (ii) is selected from

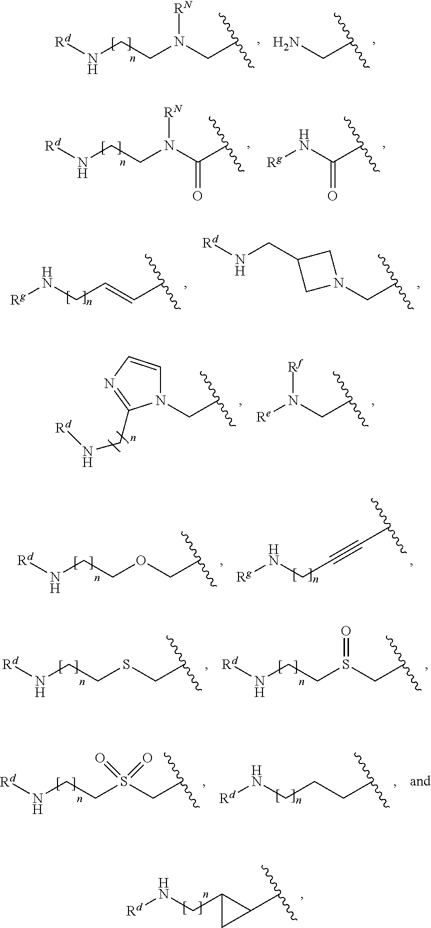

wherein
each substitutable carbon atom in the aforementioned groups is independently unsubstituted or substituted with one or more, same or different substituents selected from halogen, or two substituents together form C(═O);
R^{N} is H, C₁-C₄-alkyl, C₁-C₄-haloalkyl, NR^{a}R^{b}, cyclopropyl-C₁-C₄-alkyl, or a 5- or 6-membered saturated, partially or fully unsaturated or aromatic carbocyclyl, or heterocyclyl, wherein the aforementioned heterocyclic ring comprises one or more, same or different heteroatoms selected from O, N and S, wherein said N-and/or S-atoms are independently oxidized or non-oxidized and wherein each substitutable carbon or heteroatom in the aforementioned groups is independently unsubstituted or substituted with one or more, same or different substituents R^{x}; and
wherein
R^{a}, R^{b} are independently of each other selected from H, C₁-C₄-alkyl, and phenyl;
R^{c} is H, C₁-C₄-alkyl, NR^{a}R^{b}, NR^{a}R^{b}—C₁-C₄-alkyl, benzyl, or a 5- or 6-membered saturated heterocyclyl, wherein the aforementioned heterocyclic ring comprises one or more, same or different heteroatoms selected from O, N and S, and wherein said N-and/or S-atoms are independently oxidized or non-oxidized;
R^{d} is H, C(═O) R^{c}, phenyl-C₁-C₄-alkyl, benzophenone-C₁-C₄-alkyl, phenoxybenzene-C₁-C₄-alkyl, N-methyl-diphenylamine-C₁-C₄-alkyl, diphenylsulfide-C₁-C₄-alkyl, or naphthalene-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae

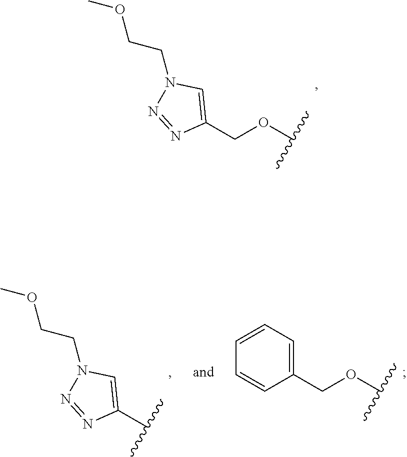

R^{e} is C₁-C₄-alkyl, C(═O) R^{c}, or a 5- or 6-membered saturated heterocyclyl, or heterocyclyl-C₁—R^{e} C₄-alkyl, wherein said heterocyclic rings comprise one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x};
R^{f} is H, or C₁-C₄-alkyl; or
R^{e} and R^{f} together with the nitrogen atom to which they are bonded form a 7- to 10-membered saturated spiro-heterocyclyl, wherein the aforementioned heterocyclic ring comprises one or more, same or different heteroatoms selected from O, N and S, and wherein said N-and/or S-atoms are independently oxidized or non-oxidized;
R^{g} is H, C(═O) R^{c}, phenyl-C₁-C₄-alkyl, benzophenone-C₁-C₄-alkyl, phenoxybenzene-C₁-C₄-alkyl, N-methyl-diphenylamine-C₁-C₄-alkyl, diphenylsulfide-C₁-C₄-alkyl, or naphthalene-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae

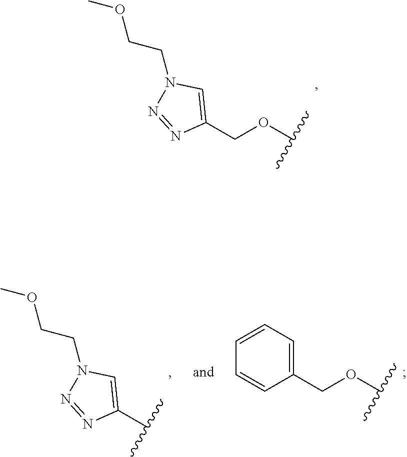

R^{x} is halogen, C₁-C₄-alkyl, C(═O) R^{c}, or phenyl-C₁-C₄-alkyl, or two R^{x} form ═O; and
n is 1, 2, 3 or 4,
for use in the treatment of prostate cancer.

In the following, preferred embodiments of the substituents of the compounds of formula (I) according to the second aspect of the present invention are described in further detail. It is to be understood that each preferred embodiment is relevant on its own as well as in combination with other preferred embodiments. Furthermore, it is to be understood that the preferences in each case also apply to the salts, stereoisomers, tautomers, and N-oxides of the compounds according to formula (I) of the invention. Further, it is to be understood that the following preferred embodiments of the substituents of the compounds of formula (I) relate to the compound of formula (I) for use in the treatment of prostate cancer.

As indicated above, in a second aspect the present invention relates to a compound of formula (I)

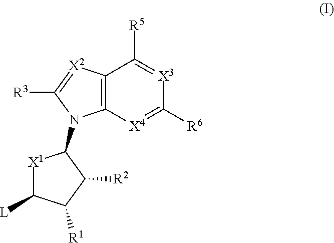

or a salt, stereoisomer, tautomer or N-oxide thereof,

wherein
X¹ is CH₂, N or O;
X² is CR⁴, or N;
X³ is CH, or N; and
X⁴ is CH, or N.

In a preferred embodiment, the present invention relates to a compound of formula (I)

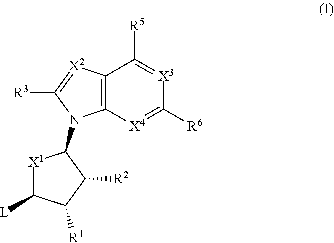

wherein the following substituent meanings are preferred for X¹, X², X³ and X⁴:
X¹ is CH₂, or O;
X² is CR⁴:
X³ is N; and
X⁴ is N.

Thus, in a more preferred embodiment, the compound of formula (I) is a compound of formula (Ia) or formula (Ib)

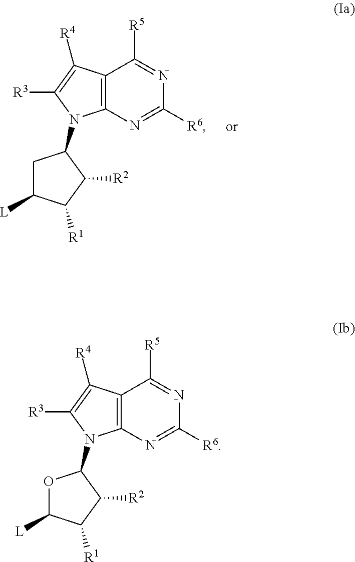

In connection with the above preferred and more preferred embodiments it is to be understood that R¹, R², R³, R⁴, R⁵, R⁶ and L are as defined above with regard to the compound of formula (I) for use in the treatment of prostate cancer of the second aspect of the present invention.

In a preferred embodiment of the present invention with regard to the compounds of formula (I), preferably with regard to the compounds of formula (Ia) and (Ib) the following substituent meanings with regard to R¹, R², R³ and R⁶ are preferred:
R¹, R² are independently of each other selected from H, halogen and OH;
R³ is H, or C₁-C₄-alkyl; and
R⁶ is H, halogen, C₁-C₄-alkyl, NR^{a}R^{b}, or OR^{c}.

In another preferred embodiment of the present invention with regard to the compounds of formula (I), preferably with regard to the compounds of formula (Ia) and (Ib) the following substituent meanings with regard to R¹, R², R³ and R⁶ are preferred:
R¹, R² are independently of each other selected from halogen and OH;
R³ is H; and
R⁶ is H, halogen, or C₁-C₄-alkyl.

In another preferred embodiment of the present invention with regard to the compounds of formula (I), preferably with regard to the compounds of formula (Ia) and (Ib) the following substituent meanings with regard to R¹, R², R³ and R⁶ are preferred:
R¹, R² are independently of each other selected from F, C₁, Br and OH; R³ is H; and
R⁶ is H, halogen, or C₁-C₄-alkyl.

In a particularly preferred embodiment of the present invention with regard to the compounds of formula (I), preferably with regard to the compounds of formula (Ia) and (Ib) the following substituent meanings with regard to R¹, R², R³ and R⁶ are preferred:
R¹, R² are OH;
R³ is H; and
R⁶ is H, halogen, or C₁-C₄-alkyl.

In another particularly preferred embodiment of the present invention with regard to the compounds of formula (I), preferably with regard to the compounds of formula (Ia) and (Ib) the following substituent meanings with regard to R¹, R², R³ and R⁶ are preferred:
R¹, R² are OH;
R³ is H; and
R⁶ is H, Br, C₁, or C₁-C₄-alkyl.

In another particularly preferred embodiment of the present invention with regard to the compounds of formula (I), preferably with regard to the compounds of formula (Ia) and (Ib) the following substituent meanings with regard to R¹, R², R³ and R⁶ are preferred:
R¹, R² are OH;
R³ is H; and
R⁶ is H, Cl, or C₁-C₄-alkyl.

In another particularly preferred embodiment of the present invention with regard to the compounds of formula (I), preferably with regard to the compounds of formula (Ia) and (Ib) the following substituent meanings with regard to R¹, R², R³ and R⁶ are preferred:
R¹, R² are OH;
R³ is H; and
R⁶ is H, or C₁-C₄-alkyl.

Thus, in a more particularly preferred embodiment of the present invention with regard to the compounds of formula (I), preferably with regard to the compounds of formula (Ia) and (Ib) the following substituent meanings with regard to R¹, R², R³ and R⁶ are preferred:
R¹, R² are OH;
R³ is H; and
R⁶ is H.

Thus, in another more particularly preferred embodiment of the present invention with regard to the compounds of formula (I), preferably with regard to the compounds of formula (Ia) and (Ib) the following substituent meanings with regard to R¹, R², R³ and R⁶ are preferred:
R¹, R² are OH;
R³ is H; and
R⁶ is H, or C₁.

In connection with the above preferred embodiments it is to be understood that R⁴, R⁵, R^{a}, R^{b}, R^{c} and L are as defined above with regard to the compound of formula (I) for use in the treatment of prostate cancer of the second aspect of the present invention or as defined further below.

Thus, in an even more preferred embodiment of the present invention, the compound of formula (I) is a compound according to formula (I.1a), (1.2a) or (I.1b)

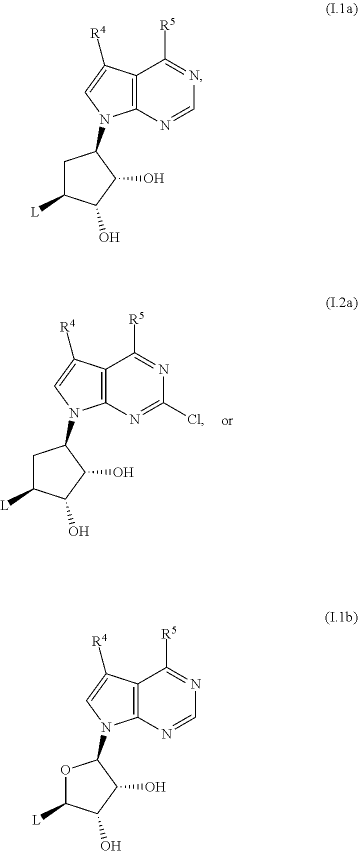

wherein R⁴, R⁵ and L are as defined above with regard to the compound of formula (I) of the second aspect of the present invention or further below.

In another more preferred embodiment of the present invention, the compound of formula (I) is a compound according to formula (I.1a) or (1.2a).

In a preferred embodiment of the present invention with regard to the compounds of formula (I), preferably with regard to the compounds of formula (Ia) and (Ib), and more preferably with regard to the compounds of formula (I.1a), (1.2a) and (I.1b) the following substituent meanings with regard to R⁴ and R⁵ are preferred:
R⁴ is H, CN, halogen, C₁-C₄-alkyl, C₂-C₄-alkenyl, phenyl, or a 3- to 6-membered saturated, partially or fully unsaturated, or aromatic carbocyclyl, or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more same or different substituents R^{x};
R⁵ is H, halogen, C₁-C₄-haloalkyl, C₁-C₄-alkyl, or NR^{a}R^{b}; or
R⁴ and R⁵ together with the atoms to which they are bonded form a 7- to 10-membered partially or fully unsaturated carbocyclyl or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x}.

In another preferred embodiment of the present invention with regard to the compounds of formula (I), preferably with regard to the compounds of formula (Ia) and (Ib), and more preferably with regard to the compounds of formula (I.1a), (1.2a) and (I.1b) the following substituent meanings with regard to R⁴ and R⁵ are preferred:
R⁴ is H, CN, halogen, C₁-C₄-alkyl, C₂-C₄-alkenyl, phenyl, or a 3- to 6-membered saturated, partially or fully unsaturated, or aromatic carbocyclyl, or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more same or different substituents R^{x};
R⁵ is H, halogen, C₁-C₄-alkyl, or NR^{a}R^{b}; or
R⁴ and R⁵ together with the atoms to which they are bonded form a 7- to 10-membered partially or fully unsaturated carbocyclyl or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x}.

In a more preferred embodiment of the present invention with regard to the compounds of formula (I), preferably with regard to the compounds of formula (Ia) and (Ib), and more preferably with regard to the compounds of formula (I.1a), (1.2a) and (I.1b) the following substituent meanings with regard to R⁴ and R⁵ are preferred:
R⁴ is H, halogen, C₁-C₄-alkyl, C₂-C₄-alkenyl, phenyl, or a 3- to 6-membered fully unsaturated or aromatic heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more same or different substituents R^{x};
R⁵ is H, C₁-C₄-alkyl, or NR^{a}R^{b}; or
R⁴ and R⁵ together with the atoms to which they are bonded form a 7- to 10-membered partially or fully unsaturated carbocyclyl or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x}.

In a more preferred embodiment of the present invention with regard to the compounds of formula (I), preferably with regard to the compounds of formula (Ia) and (Ib), and more preferably with regard to the compounds of formula (I.1a), (1.2a) and (I.1b) the following substituent meanings with regard to R⁴ and R⁵ are preferred:
R⁴ is H, halogen, C₁-C₄-alkyl, C₂-C₄-alkenyl, phenyl, or a 3- to 6-membered aromatic heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more same or different substituents R^{x}; R⁵ is H, C₁-C₄-alkyl, or NR^{a}R^{b}; or R⁴ and R⁵ together with the atoms to which they are bonded form a 7- to 10-membered partially or fully unsaturated carbocyclyl or heterocyclyl, wherein the aforementioned heterocyclic ring comprises one or more, same or different heteroatoms selected from O, N and S, wherein said N- and/or S-atoms are independently oxidized or non-oxidized, and wherein each substitutable carbon or heteroatom in the aforementioned groups is independently unsubstituted or substituted with one or more, same or different substituents R^{x}.

In a particularly preferred embodiment of the present invention with regard to the compounds of formula (I), preferably with regard to the compounds of formula (Ia) and (Ib), and more preferably with regard to the compounds of formula (I.1a), (1.2a) and (I.1b) the following substituent meanings with regard to R⁴ and R⁵ are preferred:
R⁴ is H, Br, C₁-C₂-alkyl, C₂-C₃-alkenyl, or a 5-membered fully unsaturated or aromatic heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more same or different substituents R^{x}; and
R⁵ is H, or NR^{a}R^{b}.

In connection with the above preferred embodiments it is to be understood that R^{a}, R^{b} and R^{x} are as defined above with regard to the compounds of formula (I) of the second aspect of the present invention.

Furthermore, in connection with the above preferred embodiments it is to be understood that R^{x} is as defined above with regard to the compounds of formula (I) of the second aspect of the present invention, preferably
R^{x} is halogen, C₁-C₄-alkyl, C(═O) R^{c}, or phenyl-C₁-C₄-alkyl, or two R^{x} form ═O.

More preferably, in connection with the above preferred embodiments R^{x} has the following preferred substituent meanings:
R^{x} is Cl, C₁-C₂-alkyl, or phenyl-C₁-C₂-alkyl, more preferably
R^{x} is C₁, C₁-alkyl, or phenyl-C₁-alkyl.

Furthermore, in connection with the above preferred embodiments it is to be understood that R^{a} and R^{b} are as defined above with regard to the compounds of formula (I) of the second aspect of the present invention, preferably R^{a}, R^{b} are independently of each other selected from H, C₁-C₄-alkyl, and phenyl, more preferably
R^{a}, R^{b} are independently of each other selected from H, and C₁-alkyl.

In a preferred embodiment of the present invention with regard to the compounds of formula (I), preferably with regard to the compounds of formula (Ia) and (Ib), and more preferably with regard to the compounds of formula (I.1a), (1.2a) and (I.1b) the following substituent meanings with regard to L are preferred:
L (i) is a 4- to 6-membered saturated, partially or fully unsaturated or aromatic heterocyclyl, or heterocyclyl-C₁-C₄-alkyl, wherein the aforementioned heterocyclic rings comprise one or more, same or different heteroatoms selected from O, N and S, and wherein said N-and/or S-atoms are independently oxidized or non-oxidized, and wherein each substitutable carbon or heteroatom in the aforementioned groups is independently unsubstituted or substituted with one or more, same or different substituents R^{Y}, or
   a 5- or 6-membered aromatic carbocyclyl, wherein the aforementioned carbocyclic ring is independently unsubstituted or substituted with one or more, same or different substituents R^{Y}; or
   a 7- to 10-membered aromatic carbobicyclyl or heterobicyclyl, wherein the aforementioned heterobicyclic ring comprises one or more, same or different heteroatoms selected from O, N and S, and wherein said N-and/or S-atoms are independently oxidized or non-oxidized, and wherein each substitutable carbon or heteroatom in the aforementioned groups is independently unsubstituted or substituted with one or more, same or different substituents R^{Y}; or
   (ii) is selected from

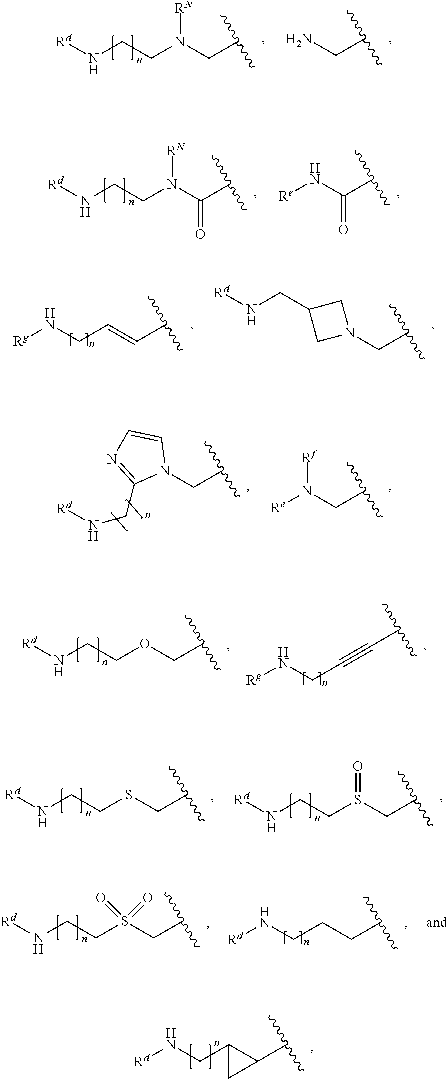

wherein
each substitutable carbon atom in the aforementioned groups is independently unsubstituted or substituted with one or more, same or different substituents selected from halogen, or two substituents together form C(═O);
R^{N} is H, C₁-C₄-alkyl, C₁-C₄-haloalkyl, NR^{a}R^{b}, cyclopropyl-C₁-C₄-alkyl, or a 5- or 6-membered saturated, partially or fully unsaturated or aromatic carbocyclyl, or heterocyclyl, wherein the aforementioned heterocyclic ring comprises one or more, same or different heteroatoms selected from O, N and S, wherein said N-and/or S-atoms are independently oxidized or non-oxidized and wherein each substitutable carbon or heteroatom in the aforementioned groups is independently unsubstituted or substituted with one or more, same or different substituents R^{x}; and wherein
R^{a}, R^{b} are independently of each other selected from H, C₁-C₄-alkyl, and phenyl;
R^{c} is H, C₁-C₄-alkyl, NR^{a}R^{b}, NR^{a}R^{b}—C₁-C₄-alkyl, benzyl, or a 5- or 6-membered saturated heterocyclyl, wherein the aforementioned heterocyclic ring comprises one or more, same or different heteroatoms selected from O, N and S, and wherein said N-and/or S-atoms are independently oxidized or non-oxidized;
R^{d} is H, C(═O) R^{c}, phenyl-C₁-C₄-alkyl, benzophenone-C₁-C₄-alkyl, phenoxybenzene-C₁-C₄-alkyl, N-methyl-diphenylamine-C₁-C₄-alkyl, diphenylsulfide-C₁-C₄-alkyl, or naphthalene-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae

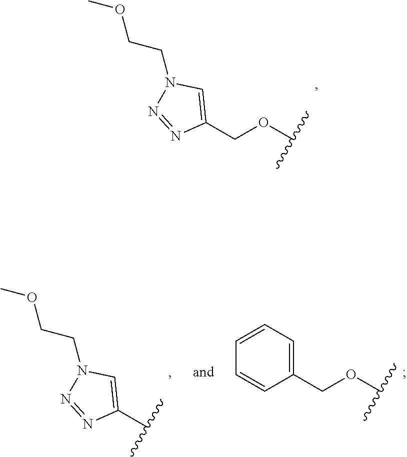

R^{e} is C₁-C₄-alkyl, C(═O) R^{c}, or a 5- or 6-membered saturated heterocyclyl, or heterocyclyl-C₁-C₄-alkyl, wherein said heterocyclic rings comprise one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x};
R^{f} is H, or C₁-C₄-alkyl; or
R^{e} and R^{f} together with the nitrogen atom to which they are bonded form a 7- to 10-membered saturated spiro-heterocyclyl, wherein the aforementioned heterocyclic ring comprises one or more, same or different heteroatoms selected from O, N and S, and wherein said N-and/or S-atoms are independently oxidized or non-oxidized;
R^{g} is H, C(═O) R^{c}, phenyl-C₁-C₄-alkyl, benzophenone-C₁-C₄-alkyl, phenoxybenzene-C₁-C₄-alkyl, N-methyl-diphenylamine-C₁-C₄-alkyl, diphenylsulfide-C₁-C₄-alkyl, or naphthalene-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae

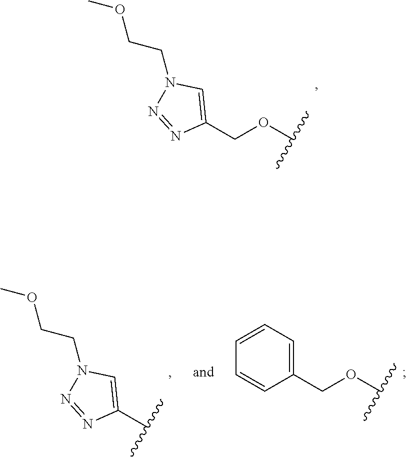

R^{h} is C₁-C₄-alkyl, or phenyl;
Rⁱ is C₁-C₄-alkyl, or phenyl;
R^{x} is halogen, C₁-C₄-alkyl, C(═O) R^{c}, S(═O) 2R^{h}, ORⁱ, or phenyl-C₁-C₄-alkyl, or two R^{x} form ═O;
R^{Y} is halogen, NH₂, OH, C₁-C₄-alkyl, C₁-C₄-haloalkyl, C₂-C₆-alkenyl, C₁-C₄-alkoxy, C₁-C₄-haloalkoxy, cyclopropyl, cyclopropyl-C₁-C₄-alkyl, phenyl-C₁-C₄-alkyl, two R^{Y} form ═O, or a 4- to 6-membered saturated, partially or fully unsaturated or aromatic carbocyclyl, carbocyclyl-C₁-C₄-alkyl, heterocyclyl or heterocyclyl-C₁-C₄-alkyl, wherein the aforementioned heterocyclic rings comprise one or more, same or different heteroatoms selected from O, N and S, wherein said N-and/or S-atoms are independently oxidized or non-oxidized, and wherein each substitutable carbon in the aforementioned groups is independently unsubstituted or substituted with one or more, same or different substituents selected from NH₂, or two substituents form ═O. In another preferred embodiment of the present invention with regard to the compounds of formula (I), preferably with regard to the compounds of formula (Ia) and (Ib), and more preferably with regard to the compounds of formula (I.1a), (1.2a) and (I.1b) the following substituent meanings with regard to L are preferred:
L (i) is a 4- to 6-membered saturated heterocyclyl, or heterocyclyl-C₁-C₄-alkyl, wherein the aforementioned heterocyclic rings comprise one or more, same or different heteroatoms selected from O, N and S, and wherein said N-and/or S-atoms are independently oxidized or non-oxidized, or
   (ii) is selected from

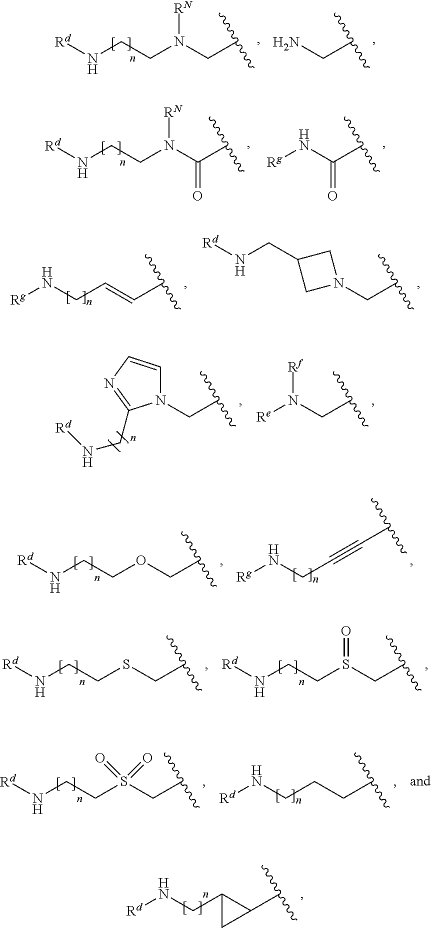

wherein
each substitutable carbon atom in the aforementioned groups is independently unsubstituted or substituted with one or more, same or different substituents selected from halogen, or two substituents together form C(═O);

wherein
R^{N} is H, C₁-C₄-alkyl, C₁-C₄-haloalkyl, NR^{a}R^{b}, cyclopropyl-C₁-C₄-alkyl, or a 5- or 6-membered saturated, partially or fully unsaturated or aromatic carbocyclyl, or heterocyclyl, wherein the aforementioned heterocyclic ring comprises one or more, same or different heteroatoms selected from O, N and S, wherein said N-and/or S-atoms are independently oxidized or non-oxidized and wherein each substitutable carbon or heteroatom in the aforementioned groups is independently unsubstituted or substituted with one or more, same or different substituents R^{x}; and wherein
R^{a}, R^{b} are independently of each other selected from H, C₁-C₄-alkyl, and phenyl;
R^{c} is H, C₁-C₄-alkyl, NR^{a}R^{b}, NR^{a}R^{b}—C₁-C₄-alkyl, benzyl, or a 5- or 6-membered saturated heterocyclyl, wherein the aforementioned heterocyclic ring comprises one or more, same or different heteroatoms selected from O, N and S, and wherein said N-and/or S-atoms are independently oxidized or non-oxidized;
R^{d} is H, C(═O) R^{c}, phenyl-C₁-C₄-alkyl, benzophenone-C₁-C₄-alkyl, phenoxybenzene-C₁-C₄-alkyl, N-methyl-diphenylamine-C₁-C₄-alkyl, diphenylsulfide-C₁-C₄-alkyl, or naphthalene-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae

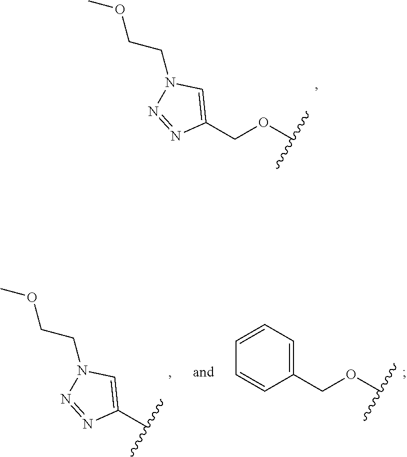

R^{e} is C₁-C₄-alkyl, C(═O) R^{c}, or a 5- or 6-membered saturated heterocyclyl, or heterocyclyl-C₁-C₄-alkyl, wherein said heterocyclic rings comprise one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x};
R^{f} is H, or C₁-C₄-alkyl; or
R^{e} and R^{f} together with the nitrogen atom to which they are bonded form a 7- to 10-membered saturated spiro-heterocyclyl, wherein the aforementioned heterocyclic ring comprises one or more, same or different heteroatoms selected from O, N and S, and wherein said N-and/or S-atoms are independently oxidized or non-oxidized;
R^{g} is H, C(═O) R^{c}, phenyl-C₁-C₄-alkyl, benzophenone-C₁-C₄-alkyl, phenoxybenzene-C₁-C₄-alkyl, N-methyl-diphenylamine-C₁-C₄-alkyl, diphenylsulfide-C₁-C₄-alkyl, or naphthalene-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae

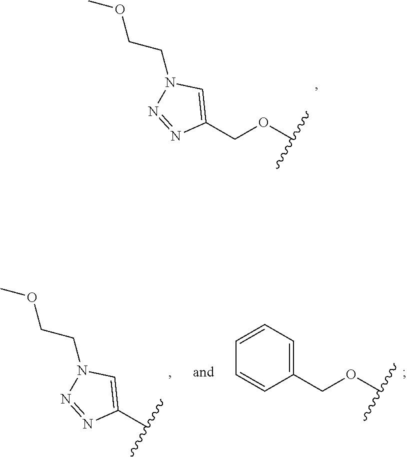

In connection with the above definitions for L, R^{d} and R^{g} it is to be understood that the curled line in the structural formula indicates the connection to the remainder of the molecule. Furthermore, it is to be understood that R^{x} is as defined above with regard to the compounds of formula (I) of the second aspect of the present invention.

In a more preferred embodiment of the present invention with regard to the compounds of formula (I), preferably with regard to the compounds of formula (Ia) and (Ib), and more preferably with regard to the compounds of formula (I.1a), (1.2a) and (I.1b) the following substituent meanings with regard to L are preferred:
L (i) is a 4- to 6-membered saturated, partially or fully unsaturated or aromatic heterocyclyl, or heterocyclyl-C₁-C₄-alkyl, wherein the aforementioned heterocyclic rings comprise one or more, same or different heteroatoms selected from O, N and S, and wherein said N-and/or S-atoms are independently oxidized or non-oxidized, and wherein each substitutable carbon or heteroatom in the aforementioned groups is independently unsubstituted or substituted with one or more, same or different substituents R^{Y}, or a 5- or 6-membered aromatic carbocyclyl, wherein the aforementioned carbocyclic ring is independently unsubstituted or substituted with one or more, same or different substituents R^{Y}; or
   (ii) is selected from

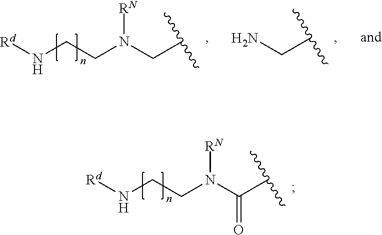

wherein
R^{N} is H, or a 5- or 6-membered saturated, partially or fully unsaturated or aromatic carbocyclyl, or heterocyclyl, wherein the aforementioned heterocyclic ring comprises one or more, same or different heteroatoms selected from O, N and S, wherein said N-and/or S-atoms are independently oxidized or non-oxidized and wherein each substitutable carbon or heteroatom in the aforementioned groups is independently unsubstituted or substituted with one or more, same or different substituents R^{x}; and
wherein
R^{d} is H, phenyl-C₁-C₄-alkyl, benzophenone-C₁-C₄-alkyl, phenoxybenzene-C₁-C₄-alkyl, or naphthalene-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae

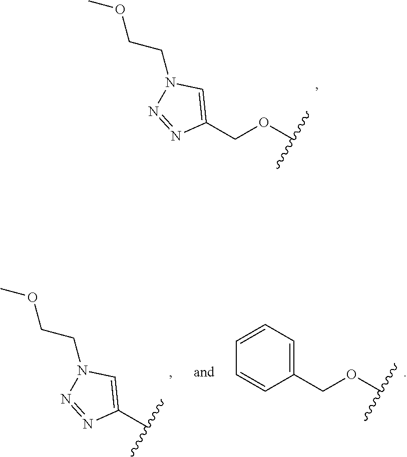

In an even more preferred embodiment of the present invention with regard to the compounds of formula (I), preferably with regard to the compounds of formula (Ia) and (Ib), and more preferably with regard to the compounds of formula (I.1a), (1.2a) and (I.1b) the following substituent meanings with regard to L are preferred:
L (i) is a 4- to 6-membered saturated, partially or fully unsaturated or aromatic heterocyclyl, wherein the aforementioned heterocyclic ring comprises one or more, same or different heteroatoms selected from O, N and S, and wherein said N-and/or S-atoms are independently oxidized or non-oxidized, and wherein each substitutable carbon or heteroatom in the aforementioned groups is independently unsubstituted or substituted with one or more, same or different substituents R^{Y}, or a 6-membered aromatic carbocyclyl, wherein the aforementioned carbocyclic ring is independently unsubstituted or substituted with one or more, same or different substituents R^{Y}; or
   (ii) is selected from

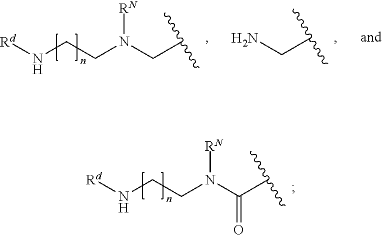

wherein
R^{N} is H, or a 5- or 6-membered saturated, partially or fully unsaturated or aromatic carbocyclyl, or heterocyclyl, wherein the aforementioned heterocyclic ring comprises one or more, same or different heteroatoms selected from O, N and S, wherein said N-and/or S-atoms are independently oxidized or non-oxidized and wherein each substitutable carbon or heteroatom in the aforementioned groups is independently unsubstituted or substituted with one or more, same or different substituents R^{x}; and
wherein
R^{d} is H, phenyl-C₁-C₄-alkyl, benzophenone-C₁-C₄-alkyl, phenoxybenzene-C₁-C₄-alkyl, or naphthalene-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae

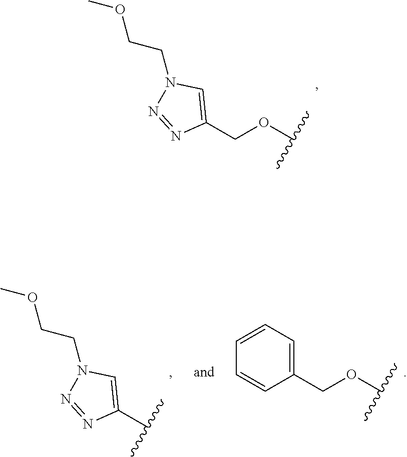

In another even more preferred embodiment of the present invention with regard to the compounds of formula (I), preferably with regard to the compounds of formula (Ia) and (Ib), and more preferably with regard to the compounds of formula (I.1a), (1.2a) and (I.1b) the following substituent meanings with regard to L are preferred:
L (i) is a 4- to 6-membered saturated, partially or fully unsaturated or aromatic heterocyclyl, wherein the aforementioned heterocyclic ring comprises one or more, same or different heteroatoms selected from O, N and S, and wherein said N-and/or S-atoms are independently oxidized or non-oxidized, and wherein each substitutable carbon or heteroatom in the aforementioned groups is independently unsubstituted or substituted with one or more, same or different substituents R^{Y}, or a 6-membered aromatic carbocyclyl, wherein the aforementioned carbocyclic ring is independently unsubstituted or substituted with one or more, same or different substituents R^{Y}; or
   (ii) is selected from

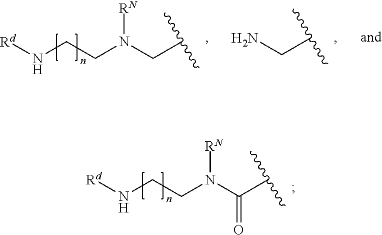

wherein
R^{N} is H, or a 5- or 6-membered saturated, partially or fully unsaturated or aromatic carbocyclyl, or heterocyclyl, wherein the aforementioned heterocyclic ring comprises one or more, same or different heteroatoms selected from O, N and S, wherein said N-and/or S-atoms are independently oxidized or non-oxidized and wherein each substitutable carbon or heteroatom in the aforementioned groups is independently unsubstituted or substituted with one or more, same or different substituents R^{x}; and
wherein
R^{d} is phenyl-C₁-C₂-alkyl, phenoxybenzene-C₁-C₂-alkyl, or naphthalene-C₁-C₂-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae

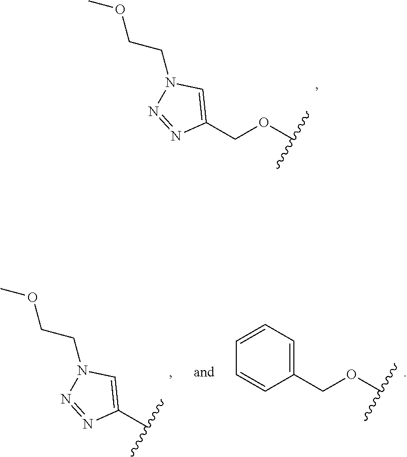

In a particularly preferred embodiment of the present invention with regard to the compounds of formula (I), preferably with regard to the compounds of formula (Ia) and (Ib), and more preferably with regard to the compounds of formula (I.1a), (1.2a) and (I.1b) the following substituent meanings with regard to L are preferred:
L (i) is a 4- to 6-membered saturated, partially or fully unsaturated or aromatic heterocyclyl, wherein the aforementioned heterocyclic ring comprises one or more, same or different heteroatoms selected from O, N and S, and wherein said N-and/or S-atoms are independently oxidized or non-oxidized, and wherein each substitutable carbon or heteroatom in the aforementioned groups is independently unsubstituted or substituted with one or more, same or different substituents R^{Y}, or a 6-membered aromatic carbocyclyl, wherein the aforementioned carbocyclic ring is independently unsubstituted or substituted with one or more, same or different substituents R^{Y}; or
   (ii) is selected from

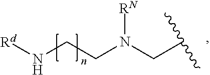

wherein
R^{N} is H, or a 5- or 6-membered saturated, partially or fully unsaturated or aromatic carbocyclyl, or heterocyclyl, wherein the aforementioned heterocyclic ring comprises one or more, same or different heteroatoms selected from O, N and S, wherein said N-and/or S-atoms are independently oxidized or non-oxidized and wherein each substitutable carbon or heteroatom in the aforementioned groups is independently unsubstituted or substituted with one or more, same or different substituents R^{x}; and
wherein
R^{d} is phenyl-C₁-C₂-alkyl, phenoxybenzene-C₁-C₂-alkyl, or naphthalene-C₁-C₂-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae

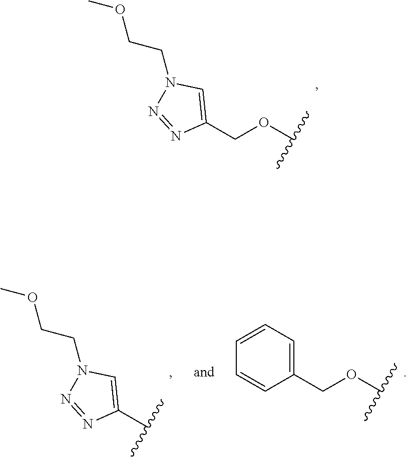

In an even more particularly preferred embodiment of the present invention with regard to the compounds of formula (I), preferably with regard to the compounds of formula (Ia) and (Ib), and more preferably with regard to the compounds of formula (I.1a), (1.2a) and (I.1b) the following substituent meanings with regard to L are preferred:
L (i) is a 4- to 6-membered saturated, partially or fully unsaturated or aromatic heterocyclyl, wherein the aforementioned heterocyclic ring comprises one or more, same or different heteroatoms selected from O, N and S, and wherein said N-and/or S-atoms are independently oxidized or non-oxidized, and wherein each substitutable carbon or heteroatom in the aforementioned groups is independently unsubstituted or substituted with one or more, same or different substituents R^{Y}, or a 6-membered aromatic carbocyclyl, wherein the aforementioned carbocyclic ring is independently unsubstituted or substituted with one or more, same or different substituents R^{Y}; or
   (ii) is selected from

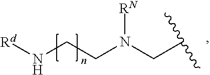

wherein
R^{N} is H; and

wherein
R^{d} is phenyl-C₁-C₂-alkyl, phenoxybenzene-C₁-C₂-alkyl, or naphthalene-C₁-C₂-alkyl, wherein each substitutable carbon or heteroatom in the aforementioned groups is independently unsubstituted or substituted with one or more, same or different substituents selected from halogen, and C₁-C₄-haloalkyl.

In connection with the above preferred embodiments, it is to be understood that R^{x} and R^{Y} are as defined above with regard to the compounds of formula (I) of the second aspect of the present invention.

Preferably, R^{x} has the following substituent meanings with regard to the above preferred embodiments:
R^{x} is halogen, C₁-C₄-alkyl, C(═O) R^{c}, or phenyl-C₁-C₄-alkyl, or two R^{x} form ═O. Furthermore, in connection with the above definitions for L and R^{d} it is to be understood that the curled line in the structural formula indicates the connection to the remainder of the molecule.

In another more preferred embodiment of the present invention with regard to the compounds of formula (I), preferably with regard to the compounds of formula (Ia) and (Ib), and more preferably with regard to the compounds of formula (I.1a), (1.2a) and (I.1b) the following substituent meanings with regard to L are preferred:
L (i) is a 4- to 6-membered saturated heterocyclyl, wherein the aforementioned heterocyclic ring comprises one or more, same or different heteroatoms selected from O, N and S, and wherein said N-and/or S-atoms are independently oxidized or non-oxidized, or
   (ii) is selected from

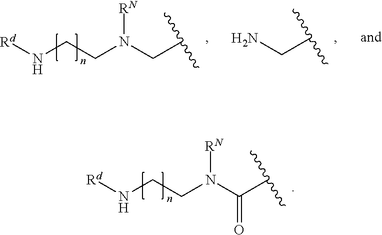

In connection with the above more preferred embodiment it is to be understood that
R^{N} is H, or a 5- or 6-membered saturated, partially or fully unsaturated or aromatic carbocyclyl, or heterocyclyl, wherein the aforementioned heterocyclic ring comprises one or more, same or different heteroatoms selected from O, N and S, wherein said N-and/or S-atoms are independently oxidized or non-oxidized and wherein each substitutable carbon or heteroatom in the aforementioned groups is independently unsubstituted or substituted with one or more, same or different substituents R^{x};
R^{d} is H, phenyl-C₁-C₄-alkyl, benzophenone-C₁-C₄-alkyl, phenoxybenzene-C₁-C₄-alkyl, or naphthalene-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae

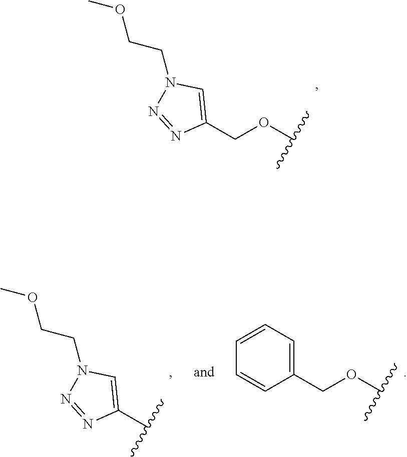

In connection with the above more preferred embodiment it is to be understood that R^{x} is as defined above with regard to the compounds of formula (I) of the second aspect of the present invention. Furthermore, in connection with the above preferred embodiments it is to be understood that R^{x} is as defined above with regard to the compounds of formula (I) of the second aspect of the present invention, preferably
R^{x} is halogen, C₁-C₄-alkyl, C(═O) R^{c}, or phenyl-C₁-C₄-alkyl, or two R^{x} form ═O.

Furthermore, in connection with the above definitions for L and R^{d} it is to be understood that the curled line in the structural formula indicates the connection to the remainder of the molecule.

Furthermore, it is to be understood that
n is 1, 2, 3 or 4, preferably
n is 1, 2, or 3, more preferably
n is 1 or 2.

In the following, preferred embodiments of the substituents of the compounds of formula (I) according to the third aspect of the present invention are described in further detail. It is to be understood that each preferred embodiment is relevant on its own as well as in combination with other preferred embodiments. Furthermore, it is to be understood that the preferences in each case also apply to the salts, stereoisomers, tautomers, and N-oxides of the compounds according to formula (I) of the invention.

As indicated above with regard to the objects and summary of the invention, in a third aspect the present invention relates to a compound of formula (I)

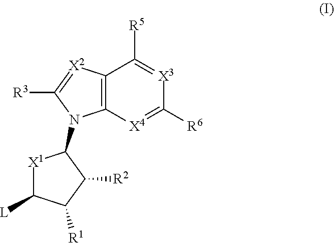

or a salt, stereoisomer, tautomer or N-oxide thereof,

wherein
X¹ is CH₂, or N;
X² is CR⁴ or N;
X³ is CH or N;
X⁴ is CH, or N.

In a preferred embodiment, the present invention relates to a compound of formula (I)

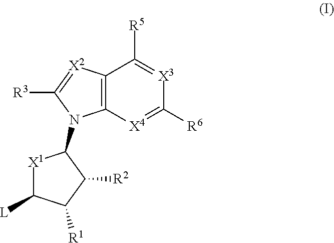

wherein the following substituent meanings are preferred for X¹, x2, x3 and X⁴:
X¹ is CH₂;
X² is CR⁴;
X³ is CH or N; and
X⁴ is CH or N.

In a more preferred embodiment, the present invention relates to a compound of formula (I)

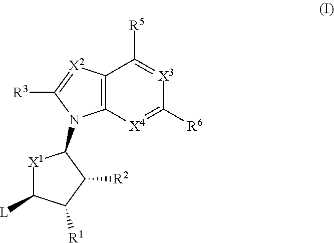

wherein the following substituent meanings are preferred for X¹, X², x3 and X⁴:
X¹ is CH₂;
X² is CR⁴;
X³ is N; and
X⁴ is N.

Thus, in an even more preferred embodiment, the compound of formula (I) is a compound of formula (Ia)

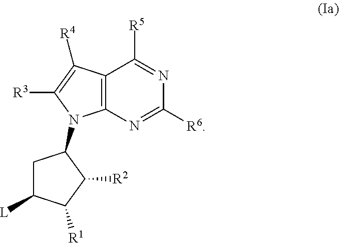

In connection with the above preferred and more preferred embodiments it is to be understood that R¹, R², R³, R⁴, R⁵, R⁶ and L are as defined above with regard to the compounds of formula (I) of the third aspect of the present invention or as defined further below.

Furthermore, it is to be understood with regard to the above preferred and more preferred embodiments that if X² is N or CR⁴, wherein R⁴ is H, then R⁵ is not H or NH₂, preferably it is to be understood that if X² is CR⁴, wherein R⁴ is H, then R⁵ is not H or NH₂.

In a preferred embodiment of the present invention with regard to the compounds of formula (I), preferably with regard to the compounds of formula (Ia) of the third aspect as defined above, the following substituent meanings with regard to R¹, R², R³, R⁴, R⁵ and R⁶ are preferred:
R¹, R² are independently of each other selected from H, halogen and OH;
R³ is H, or C₁-C₄-alkyl;
R⁴ is H, CN, halogen, C₁-C₄-alkyl, C₂-C₄-alkenyl, phenyl, or a 3- to 6-membered saturated, partially or fully unsaturated, or aromatic carbocyclyl, or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more same or different substituents R^{x};
R⁵ is H, C₁-C₄-alkyl, C₁-C₄-haloalkyl, or NR^{a}R^{b}; or
R⁴ and R⁵ together with the atoms to which they are bonded form a 7- to 10-membered partially or fully unsaturated carbocyclyl or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x}; and
R⁶ is H, halogen, C₁-C₄-alkyl, NR^{a}R^{b}, or OR^{c}.

In a more preferred embodiment of the present invention with regard to the compounds of formula (I), preferably with regard to the compounds of formula (Ia) of the third aspect as defined above, the following substituent meanings with regard to R¹, R², R³, R⁴, R⁵ and R⁶ are preferred:
R¹, R² are OH;
R³ is H;
R⁴ is H, halogen, C₁-C₄-alkyl or
   a 5-membered fully unsaturated or aromatic heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more same or different substituents R^{x}:
R⁵ is H, or NR^{a}R^{b}; or
R⁴ and R⁵ together with the atoms to which they are bonded form a 7- to 10-membered partially or fully unsaturated carbocyclyl or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x}; and
R⁶ is H, or halogen.

In another more preferred embodiment of the present invention with regard to the compounds of formula (I), preferably with regard to the compounds of formula (Ia) of the third aspect as defined above, the following substituent meanings with regard to R¹, R², R³, R⁴, R⁵ and R⁶ are preferred:
R¹, R² are OH;
R³ is H;
R⁴ is H, Br, CH₃, CH₂CH₃ Or a 5-membered fully unsaturated or aromatic heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more same or different substituents R^{x};
R⁵ is H, or NR^{a}R^{b}; or
R⁴ and R⁵ together with the atoms to which they are bonded form a 7- to 10-membered partially or fully unsaturated carbocyclyl or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x};
R⁶ is H, or Cl.

In an even more preferred embodiment of the present invention with regard to the compounds of formula (I), preferably with regard to the compounds of formula (Ia) of the third aspect as defined above, the following substituent meanings with regard to R¹, R², R³, R⁴, R⁵ and R⁶ are preferred:
R¹, R² are OH;
R³ is H;
R⁴ is H, Br, CH₃, CH₂CH₃ Or a 5-membered fully unsaturated or aromatic heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more same or different substituents R^{x};
R⁵ is H, or NR^{a}R^{b}; and
R⁶ is H, or C₁.

In connection with the above preferred embodiments it is to be understood that R^{a}, R^{b}, R^{c}, R^{x} and L are as defined above with regard to the compounds of formula (I) of the third aspect of the present invention. In addition, it is to be understood that the remaining substituents such as X¹, X², X³, X⁴ are as defined above with regard to the compound of formula (I) of the third aspect. Furthermore, in connection with the above preferred embodiments it is to be understood that if X² is N or CR⁴, wherein R⁴ is H, then R⁵ is not H or NH₂, preferably it is to be understood that if X² is CR⁴, wherein R⁴ is H, then R⁵ is not H or NH₂.

Thus, in an even more preferred embodiment of the present invention, the compound of formula (I), preferably the compound of formula (Ia) of the third aspect, is a compound according to formula (I.1a), or (1.2a)

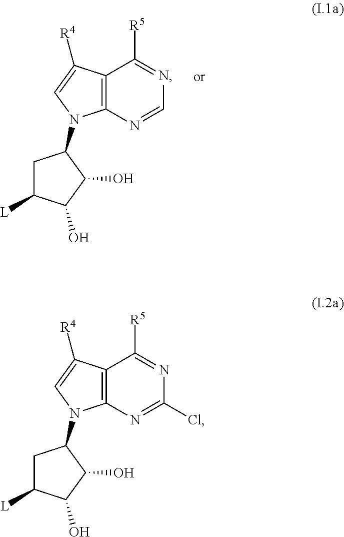

wherein
R⁴ is H, Br, CH₃, CH₂CH₃ Or a 5-membered fully unsaturated or aromatic heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more same or different substituents R^{x}; and
R⁵ is H, or NR^{a}R^{b}.

In connection with the above more preferred embodiment it is to be understood that R^{a}, R^{b}, R^{x} and L are as defined above with regard to the compounds of formula (I) of the third aspect of the present invention or as defined further below.

In a preferred embodiment of the present invention, R⁴ and R⁵ have the following substituent meanings with regard to the compounds of formula (I), preferably with regard to the compounds of formula (Ia) and more preferably with regard to the compounds of formula (I.1a), and (1.2a)
R⁴ is H, Br, CH₃, CH₂CH₃ Or a 5-membered fully unsaturated or aromatic heterocyclyl selected from the following structural formulae

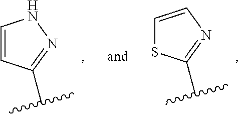 [SMILES: *c1cc[nH]n1 ; *c1nccs1]

wherein each substitutable carbon or heteroatom in the aforementioned groups is independently unsubstituted or substituted with one or more same or different substituents R^{x}; and R⁵ is H, or NR^{a}R^{b}.

In connection with the above structural formulae of R⁴ it is to be understood that the curled line in the structural formulae indicates the connection to the remainder of the molecule.

Furthermore, in connection with the above preferred embodiments it is to be understood that if X² is N or CR⁴, wherein R⁴ is H, then R⁵ is not H or NH₂, preferably it is to be understood that if X² is CR⁴, wherein R⁴ is H, then R⁵ is not H or NH₂.

Further, in connection with the above preferred embodiments regarding the substituents R⁴ and R⁵, it is to be understood that R^{x}, R^{a} and R^{b} have the following preferred substituent meanings:
R^{x} is C₁-C₄-alkyl, C(═O) R^{c}, S(═O) 2R^{h}, ORⁱ, or phenyl-C₁-C₄-alkyl; and R^{a}, R^{b} are independently of each other selected from H, C₁-C₄-alkyl and phenyl.

Preferably, R^{x}, R^{a} and R^{b} have the following preferred substituent meanings:
R^{x} is C₁-C₄-alkyl, C(═O) R^{c}, or phenyl-C₁-C₄-alkyl; and
R^{a}, R^{b} are independently of each other selected from H, C₁-C₄-alkyl and phenyl.

More preferably, R^{x}, R^{a} and R^{b} have the following preferred substituent meanings:
R^{x} is CH₃, CH₂CH₃, or phenyl-C₁-C₂-alkyl; and R^{a}, R^{b} are independently of each other selected from H, CH₃ and CH₂CH₃.

Even more preferably, R^{x}, R^{a} and R^{b} have the following preferred substituent meanings:
R^{x} is CH₃, CH₂CH₃, or phenyl-C₁-alkyl; and
R^{a}, R^{b} are independently of each other selected from H, and CH₃.

In connection with the above preferred embodiments it is to be understood that if X² is CR⁴, wherein R⁴ is H, then R⁵ is not H or NH₂.

In a preferred embodiment of the present invention with regard to the compounds of formula (I), preferably with regard to the compounds of formula (Ia), and more preferably with regard to the compounds of formula (I.1a) and (1.2a) of the third aspect as defined above, the following substituent meanings with regard to L are preferred:
L (i) is a 4- to 6-membered saturated, partially or fully unsaturated or aromatic heterocyclyl, wherein the aforementioned heterocyclic ring comprises one or more, same or different heteroatoms selected from O, N and S, and wherein said N-and/or S-atoms are independently oxidized or non-oxidized, and wherein each substitutable carbon or heteroatom in the aforementioned groups is independently unsubstituted or substituted with one or more, same or different substituents R^{Y}, or
   a 5- or 6-membered aromatic carbocyclyl, wherein the aforementioned carbocyclic ring is independently unsubstituted or substituted with one or more, same or different substituents R^{Y}; or
   a 7- to 10-membered aromatic carbobicyclyl or heterobicyclyl, wherein the aforementioned heterobicyclic ring comprises one or more, same or different heteroatoms selected from O, N and S, and wherein said N-and/or S-atoms are independently oxidized or non-oxidized, and wherein each substitutable carbon or heteroatom in the aforementioned groups is independently unsubstituted or substituted with one or more, same or different substituents R^{Y}; or
   (ii) is selected from

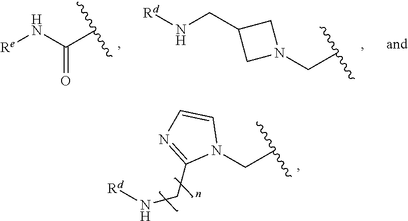

wherein
each substitutable carbon atom in the aforementioned groups is independently unsubstituted or substituted with one or more, same or different substituents selected from halogen, or two substituents together form C(═O).

In connection with the above preferred embodiment, it is to be understood that R^{e}, R^{d}, R^{Y} and n are as defined above with regard to the compounds of formula (I) of the third aspect or as defined further below.

Preferably, the substituents R^{d}, R^{e} as well as n have the following preferred meanings:
R^{d} is benzophenone-C₁-C₄-alkyl, phenoxybenzene-C₁-C₄-alkyl, N-methyl-diphenylamine-C₁-C₄-alkyl, or diphenylsulfide-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae

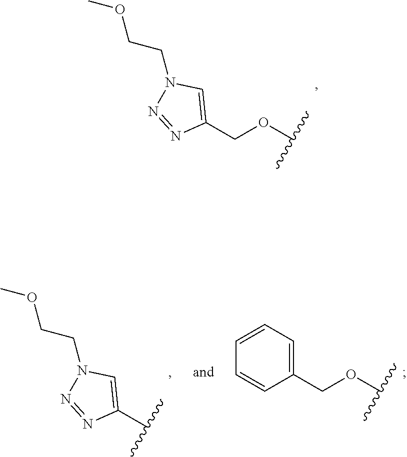

R^{e} is a 5- or 6-membered saturated heterocyclyl, wherein said heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x};
wherein
R^{x} is C₁-C₄-alkyl, C(═O) R^{c}, S(═O) 2R^{h}, ORⁱ, or phenyl-C₁-C₄-alkyl; and
n is 1, 2, 3 or 4.

Preferably, R^{x} has the following preferred substituent meanings with regard to the above preferred embodiments:
R^{x} is C₁-C₄-alkyl, C(═O) R^{c}, or phenyl-C₁-C₄-alkyl.

In a more preferred embodiment of the present invention with regard to the compounds of formula (I), preferably with regard to the compounds of formula (Ia), and more preferably with regard to the compounds of formula (I.1a) and (1.2a) of the third aspect as defined above, the following substituent meanings with regard to L are preferred:
L (i) is a 4- to 6-membered saturated, partially or fully unsaturated or aromatic heterocyclyl, wherein the aforementioned heterocyclic ring comprises one or more, same or different heteroatoms selected from O, N and S, and wherein said N-and/or S-atoms are independently oxidized or non-oxidized, and wherein each substitutable carbon or heteroatom in the aforementioned groups is independently unsubstituted or substituted with one or more, same or different substituents R^{Y}, or
   a 5- or 6-membered aromatic carbocyclyl, wherein the aforementioned carbocyclic ring is independently unsubstituted or substituted with one or more, same or different substituents R^{Y}; or
   a 7- to 10-membered aromatic carbobicyclyl or heterobicyclyl, wherein the aforementioned heterobicyclic ring comprises one or more, same or different heteroatoms selected from O, N and S, and wherein said N-and/or S-atoms are independently oxidized or non-oxidized, and wherein each substitutable carbon or heteroatom in the aforementioned groups is independently unsubstituted or substituted with one or more, same or different substituents R^{Y}.

More preferably, with regard to the compounds of formula (I), preferably with regard to the compounds of formula (Ia), and more preferably with regard to the compounds of formula (I.1a) and (1.2a) of the third aspect as defined above, the following substituent meanings with regard to L are preferred:
L (i) is a 4- to 6-membered saturated, partially or fully unsaturated or aromatic heterocyclyl, wherein the aforementioned heterocyclic ring comprises one or more, same or different heteroatoms selected from O, N and S, and wherein said N-and/or S-atoms are independently oxidized or non-oxidized, and wherein each substitutable carbon or heteroatom in the aforementioned groups is independently unsubstituted or substituted with one or more, same or different substituents R^{Y}, or
   a 5- or 6-membered aromatic carbocyclyl, wherein the aforementioned carbocyclic ring is independently unsubstituted or substituted with one or more, same or different substituents R^{Y}.

In connection with the above preferred embodiments, it is to be understood that R^{Y} are as defined above with regard to the compounds of formula (I) of the third aspect of the present invention or as defined further below.

Even more preferably, with regard to the compounds of formula (I), preferably with regard to the compounds of formula (Ia), and more preferably with regard to the compounds of formula (I.1a) and (1.2a) of the third aspect as defined above, the following substituent meanings with regard to L are preferred:
L (i) is a 5- or 6-membered saturated, partially or fully unsaturated or aromatic heterocyclyl, wherein the aforementioned heterocyclic ring comprises one or more, same or different heteroatoms selected from O, N and S, and wherein said N-and/or S-atoms are independently oxidized or non-oxidized, and wherein each substitutable carbon or heteroatom in the aforementioned groups is independently unsubstituted or substituted with one or more, same or different substituents R^{Y}, or
   a 5- or 6-membered aromatic carbocyclyl, wherein the aforementioned carbocyclic ring is independently unsubstituted or substituted with one or more, same or different substituents R^{Y}.

In a particularly preferred embodiment, L has the following substituent meanings with regard to the compounds of formula (I), preferably with regard to the compounds of formula (Ia) and more preferably with regard to the compounds of formula (I.1a), and (1.2a) of the third aspect as defined above:
L (i) is a 5- or 6-membered saturated, partially or fully unsaturated or aromatic heterocyclyl wherein the aforementioned heterocyclic ring comprises one or more, same or different heteroatoms selected from O, N and S, and wherein said N-and/or S-atoms are independently oxidized or non-oxidized, selected from the following structural formulae

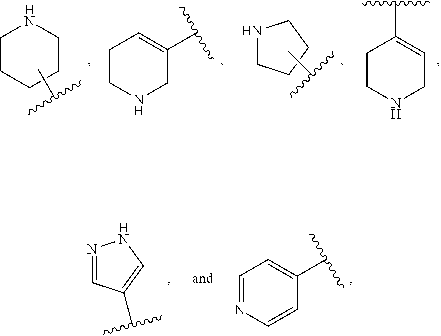

wherein each substitutable carbon or heteroatom in the aforementioned groups is independently unsubstituted or substituted with one or more, same or different substituents R^{Y}, or
L is a 6-membered aromatic carbocyclyl and has the following structural formula

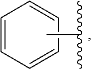

wherein the aforementioned carbocyclic ring is independently unsubstituted or substituted with one or more, same or different substituents R^{Y}.

In one preferred embodiment of the present invention, L is a 5- or 6-membered saturated, partially or fully unsaturated or aromatic heterocyclyl wherein the aforementioned heterocyclic ring comprises one or more, same or different heteroatoms selected from O, N and S, and wherein said N-and/or S-atoms are independently oxidized or non-oxidized, according to the following structural formulae

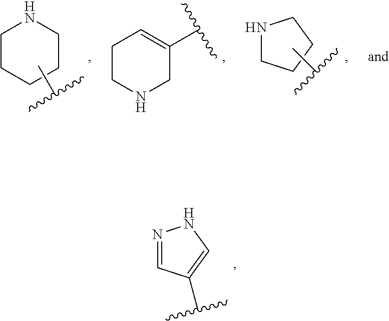

wherein the heterocyclic ring is not further substituted.

In another preferred embodiment of the present invention, L is a 6-membered aromatic carbocyclyl and has the following structural formula

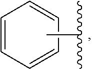

wherein the carbocyclic ring is not further substituted.

In connection with the above structural formulae of L it is to be understood that the curled line in the structural formulae indicates the connection to the remainder of the molecule.

Furthermore, in connection with the above preferred embodiments regarding the substituent L, it is to be understood that R^{Y} has the following preferred substituent meanings:
R^{Y} is halogen, NH₂, OH, C₁-C₄-alkyl, C₁-C₄-haloalkyl, C₂-C₆-alkenyl, C₁-C₄-alkoxy, C₁-C₄-haloalkoxy, cyclopropyl, cyclopropyl-C₁-C₄-alkyl, phenyl-C₁-C₄-alkyl, two R^{Y} form ═O, or a 4- to 6-membered saturated, partially or fully unsaturated or aromatic carbocyclyl, carbocyclyl-C₁-C₄-alkyl, heterocyclyl or heterocyclyl-C₁-C₄-alkyl, wherein the aforementioned heterocyclic rings comprise one or more, same or different heteroatoms selected from O, N and S, wherein said N-and/or S-atoms are independently oxidized or non-oxidized, and wherein each substitutable carbon in the aforementioned groups is independently unsubstituted or substituted with one or more, same or different substituents selected from NH₂, or two substituents form ═O.

Preferably, R^{Y} has the following preferred substituent meanings with regard to L as defined above:
R^{Y} is Cl, Br, OH, C₁-C₃-alkyl, C₁-C₂-haloalkyl, C₂-C₄-alkenyl, C₁-C₂-alkoxy, cyclopropyl, cyclopropyl-C₁-C₂-alkyl, phenyl-C₁-C₂-alkyl, or a 4- to 6-membered saturated or fully unsaturated or aromatic carbocyclyl, carbocyclyl-C₁-C₂-alkyl, heterocyclyl or heterocyclyl-C₁-C₂-alkyl, wherein the aforementioned heterocyclic rings comprise one or more, same or different heteroatoms selected from O, N and S, wherein said N-and/or S-atoms are independently oxidized or non-oxidized, and wherein each substitutable carbon in the aforementioned groups is independently unsubstituted or substituted with one or more, same or different substituents selected from NH₂, or two substituents form ═O.

More preferably, R^{Y} has the following preferred substituent meanings with regard to L as defined above:
R^{Y} is Cl, OH, C₁-C₃-alkyl, C₁-alkoxy, cyclopropyl, cyclopropyl-C₁-alkyl, C₂-haloalkyl, phenyl-C₁-C₂-alkyl, C₃-alkenyl, or a 4- to 6-membered saturated or fully unsaturated or aromatic carbocyclyl, carbocyclyl-C₁-alkyl, heterocyclyl or heterocyclyl-C₁-C₂-alkyl, wherein the aforementioned heterocyclic rings comprise one or more, same or different heteroatoms selected from O, N and S, wherein said N-and/or S-atoms are independently oxidized or non-oxidized, and wherein each substitutable carbon in the aforementioned groups is independently unsubstituted or substituted with one or more, same or different substituents selected from NH₂, or two substituents form ═O.

In a particularly preferred embodiment of the present invention, R^{Y} has the following preferred substituent meanings with regard to L as defined above:
R^{Y} is CH₃, CH(CH₃) 2, cyclopropyl-C₁-alkyl, phenyl-C₂-alkyl, or a 4- to 6-membered saturated or aromatic carbocyclyl-C₁-alkyl or heterocyclyl-C₁-C₂-alkyl, wherein the aforementioned heterocyclic ring comprises one or more, same or different heteroatoms selected from O, N and S, wherein said N-and/or S-atoms are independently oxidized or non-oxidized, and wherein each substitutable carbon in the aforementioned groups is independently unsubstituted or substituted with one or more, same or different substituents selected from NH₂, or two substituents form ═O;
more preferably
R^{Y} is CH₃, CH(CH₃) 2, cyclopropyl-C₁-alkyl, phenyl-C₂-alkyl, or a 4- to 6-membered saturated or aromatic carbocyclyl-C₁-alkyl or heterocyclyl-C₁-C₂-alkyl, wherein the aforementioned heterocyclic ring comprises one or more, same or different heteroatoms selected from O, N and S, wherein said N-and/or S-atoms are independently oxidized or non-oxidized.

In connection with the above embodiments regarding the substituent L it is to be understood that the remaining substituents of the compound of formula (I) such as X¹, X², X³ and X⁴ as well as R¹, R², R³, R⁴, R⁵ and R⁶ are as defined above with regard to the compounds of formula (I) of the third aspect.

In a fourth aspect, the present invention relates to a compound of formula (I)

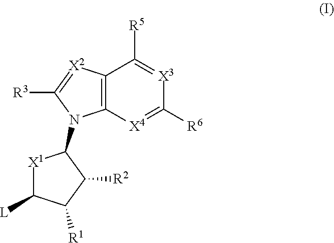

or a salt, stereoisomer, tautomer or N-oxide thereof,

wherein
X¹ is CH₂, or N;
X² is CR⁴ or N;
X³ is CH or N;
X⁴ is CH, or N;
R¹, R² are independently of each other selected from H, halogen and OH;
R³ is H, or C₁-C₄-alkyl;
R⁴ is H, CN, halogen, C₁-C₄-alkyl, C₂-C₄-alkenyl, phenyl, or a 3- to 6-membered saturated, partially or fully unsaturated, or aromatic carbocyclyl, or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more same or different substituents R^{x};
R⁵ is H, C₁-C₄-alkyl, or NR^{a}R^{b}; or
R⁴ and R⁵ together with the atoms to which they are bonded form a 7- to 10-membered partially or fully unsaturated carbocyclyl or heterocyclyl, wherein the aforementioned heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x};
R⁶ is H, halogen, C₁-C₄-alkyl, NR^{a}R^{b}, or ORG;
wherein
L (i) is a 4- to 6-membered saturated heterocyclyl, wherein the aforementioned heterocyclic ring comprises one or more, same or different heteroatoms selected from O, N and S, and wherein said N-and/or S-atoms are independently oxidized or non-oxidized, or
   (ii) is selected from

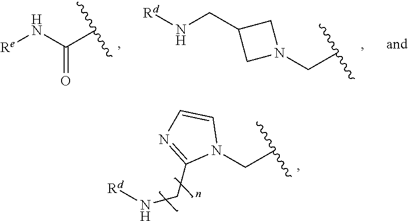

wherein
each substitutable carbon atom in the aforementioned groups is independently unsubstituted or substituted with one or more, same or different substituents selected from halogen, or two substituents together form C(═O);
wherein
R^{a}, R^{b} are independently of each other selected from H, C₁-C₄-alkyl and phenyl;
R^{c} is H, C₁-C₄-alkyl, NR^{a}R^{b}, NR^{a}R^{b}—C₁-C₄-alkyl, benzyl, or a 5- or 6-membered saturated heterocyclyl, wherein the aforementioned heterocyclic ring comprises one or more, same or different heteroatoms selected from O, N and S, and wherein said N-and/or S-atoms are independently oxidized or non-oxidized;
R^{d} is benzophenone-C₁-C₄-alkyl, phenoxybenzene-C₁-C₄-alkyl, N-methyl-diphenylamine-C₁-C₄-alkyl, or diphenylsulfide-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae

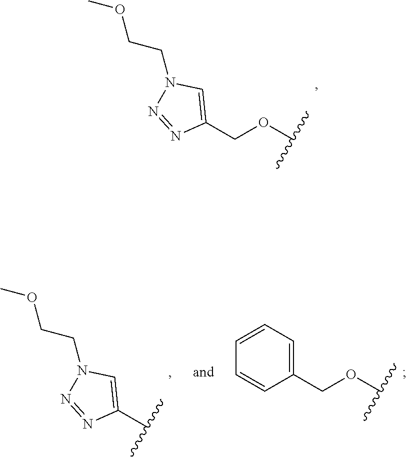

R^{e} is a 5- or 6-membered saturated heterocyclyl, wherein said heterocyclic ring comprises one or more, same or different heteroatoms selected from O, N and S, wherein said N-and/or S-atoms are independently oxidized or non-oxidized, and wherein each substitutable carbon or heteroatom in the aforementioned groups is independently unsubstituted or substituted with one or more, same or different substituents R^{x};
R^{g} is H, C(═O) R^{c}, phenyl-C₁-C₄-alkyl, benzophenone-C₁-C₄-alkyl, phenoxybenzene-C₁-C₄-alkyl, N-methyl-diphenylamine-C₁-C₄-alkyl, diphenylsulfide-C₁-C₄-alkyl, or naphthalene-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae

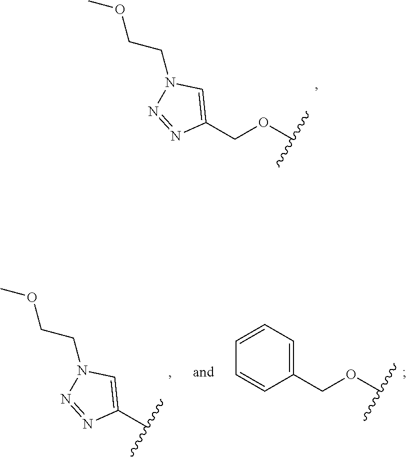

R^{x} is C₁-C₄-alkyl, C(═O) R^{c}, or phenyl-C₁-C₄-alkyl; and
n is 1, 2, 3 or 4;
with the proviso that if X² is N or CR⁴, wherein R⁴ is H, then R⁵ is not H or NH₂.

Preferred embodiments of the substituents of the compounds of formula (I) according to the fourth aspect of the present invention are described in further detail in the first aspect of the present invention. It is to be understood that each preferred embodiment is relevant on its own as well as in combination with other preferred embodiments. Furthermore, it is to be understood that the preferences in each case also apply to the salts, stereoisomers, tautomers, and N-oxides of the compounds according to formula (I) of the invention.

Thus, the preferred embodiments of the substituents of the compounds of formula (I) according to the fourth aspect are covered by the first aspect of the present invention.

### Definitions

The term “compound(s) of the present invention” is to be understood as equivalent to the term “compound(s) according to the invention”, and also covers a salt, stereoisomer, tautomer or N-oxide thereof.

The compounds according to the invention may be amorphous or may exist in one or more different crystalline states (polymorphs), which may have different macroscopic properties such as stability or show different biological properties such as activities. The present invention relates to amorphous and crystalline forms of compounds of formula (I), mixtures of different crystalline states of the compounds of formula (I), as well as amorphous or crystalline salts thereof.

Salts of the compounds according to the invention are preferably pharmaceutically acceptable salts, such as those containing counterions present in drug products listed in the US FDA orange Book database. They can be formed in a customary manner, e.g., by reacting the compound with an acid of the anion in question, if the compounds according to the invention have a basic functionality, or by reacting acidic compounds according to the invention with a suitable base. Suitable cationic counterions are in particular the ions of alkali metals, preferably lithium, sodium and potassium, of the alkaline earth metals, preferably calcium, magnesium and barium, and of the transition metals preferably manganese, copper, silver, zinc and iron, and also ammonium (NH₄+) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxy-ethylammonium, 2-(2-hydroxyethoxy)ethyl-ammonium, bis(2-hydroxyethyl) ammonium, benzyltrimethylammonium and benzyltriethylammonium, furthermore the cations of 1,4-piperazine, meglumine, benzathine and lysine.

Suitable anionic counterions are in particular chloride, bromide, hydrogensulfate, sulfate, dihydrogenphosphate, hydrogenphosphate, phosphate, nitrate, bicarbonate, carbonate, hexafluorosilicate, hexafluorophosphate, benzoate, and the anion of C₁-C₄-alkanoic acids, preferably formate, acetate, propionate and butyrate, furthermore lactate, gluconate, and the anions of poly acids such as succinate, oxalate, maleate, fumarate, malate, tartrate and citrate, furthermore sulfonate anions such as besylate (benzenesulfonate), tosylate (p-toluenesulfonate), napsylate (naphthalene-2-sulfonate), mesylate (methanesulfonate), esylate (ethanesulfonate), and ethanedisulfonate. They can be formed by reacting compounds according to the invention that have a basic functionality with an acid of the corresponding anion.

Depending on the substitution pattern, the compounds according to the invention may have one or more centers of chirality, including axial chirality. The invention provides both, pure enantiomers or pure diastereomers, of the compounds according to the invention, and their mixtures, including racemic mixtures. Suitable compounds according to the invention also include all possible geometrical stereoisomers (cis/trans isomers or E/Z isomers) and mixtures thereof. E/Z-isomers may be present with respect to e.g., an alkene, carbon-nitrogen double bond or amide groups.

Tautomers may be formed, if a substituent is present at the compound of formula (I), which allows for the formation of tautomers such as keto-enol tautomers, imine-enamine tautomers, amide-imidic acid tautomers or the like.

The term “N-oxide” includes any compound of the present invention which has at least one tertiary nitrogen atom that is oxidized to a N-oxide moiety.

The term “substituted” as used herein, means that a hydrogen atom bonded to a designated atom is replaced with a specified substituent, provided that the substitution results in a stable or chemically feasible compound. Unless otherwise indicated, a substituted atom may have one or more substituents and each substituent is independently selected.

The term “substitutable”, when used in reference to a designated atom, means that attached to the atom is a hydrogen, which can be replaced with a suitable substituent.

When it is referred to certain atoms or moieties being substituted with “one or more” substituents, the term “one or more” is intended to cover at least one substituent, e.g. 1 to 10 substituents, preferably 1, 2, 3, 4 or 5 substituents, more preferably 1, 2, or 3 substituents, most preferably 1 or 2 substituents. When neither the term “unsubstituted” nor “substituted” is explicitly mentioned concerning a moiety, said moiety is to be considered as unsubstituted.

The organic moieties mentioned in the above definitions of the variables are—like the term halogen—collective terms for individual listings of the individual group members. The prefix Cₙ-Cₘ indicates in each case the possible number of carbon atoms in the group.

The term “halogen” denotes in each case fluorine, bromine, chlorine or iodine, in particular fluorine, chlorine, or bromine.

As used herein, the term “two substituents together form (═O)” or the term “two substituents together form C(═O)” is to be understood in a way that the substitutable designated atom to which two hydrogen atoms are attached is substituted by two substituents together forming (═O). In other words, if the designated atom is a carbon atom, from which two hydrogen atoms are substituted by forming a group (═O) this results in the following moiety

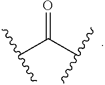 [SMILES: *C(*)=O]

The term “alkyl” as used herein denotes in each case a straight-chain or branched alkyl group having usually from 1 to 6 carbon atoms, preferably 1 to 4 carbon atoms, more preferably 1 to 3 or 1 or 2 carbon atoms. Examples of an alkyl group are methyl, ethyl, n-propyl, iso-propyl, n-butyl, 2-butyl, iso-butyl, tert-butyl, n-pentyl, 1-methylbutyl, 2-methylbutyl, 3-methylbutyl, 2,2-di-methylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethyl-butyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, and 1-ethyl-2-methylpropyl. The term “haloalkyl” as used herein denotes in each case a straight-chain or branched alkyl group having usually from 1 to 6 carbon atoms, frequently 1 to 4 carbon atoms, preferably 1 to 3 or 1 or 2 carbon atoms, wherein the hydrogen atoms of this group are partially or totally replaced with halogen atoms. Preferred haloalkyl moieties are selected from C₁-C₄-haloalkyl, more preferably from C₁-C₃-haloalkyl or C₁-C₂-haloalkyl, in particular from C₁-C₂-fluoroalkyl such as fluoromethyl, difluoromethyl, trifluoromethyl, 1-fluoroethyl, 2-fluoroethyl, 2,2-difluoroethyl, 2,2,2-trifluoroethyl, pentafluoroethyl, and the like.

The term “alkoxy” as used herein denotes in each case a straight-chain or branched alkyl group which is bonded via an oxygen atom and has usually from 1 to 4 carbon atoms, preferably 1 to 2 carbon atoms, more preferably 1 carbon atom. Examples of an alkoxy group are methoxy, ethoxy, n-propoxy, iso-propoxy, n-butyloxy, 2-butyloxy, iso-butyloxy, tert.-butyloxy, and the like.

The term “haloalkoxy” as used herein denotes in each case a straight-chain or branched alkoxy group having from 1 to 4 carbon atoms, preferably 1 to 2 carbon atoms, more preferably 1 carbon atom, wherein the hydrogen atoms of this group are partially or totally replaced with halogen atoms, in particular fluorine atoms. Preferred haloalkoxy moieties include C₁-haloalkoxy, in particular C₁-fluoroalkoxy, such as trifluoromethoxy and the like.

The term “alkenyl” as used herein denotes in each case an unsaturated hydrocarbon group having usually 2 to 6, preferably 2 to 4 carbon atoms comprising at least one carbon-carbon double bond in any position, e.g. vinyl(ethenyl), allyl(2-propen-1-yl), 1-propen-1-yl, 2-propen-2-yl, methallyl(2-methylprop-2-en-1-yl), 2-buten-1-yl, 3-buten-1-yl, 2-penten-1-yl, 3-penten-1-yl, 4-penten-1-yl, 1-methylbut-2-en-1-yl, 2-ethylprop-2-en-1-yl and the like. If geometric isomers are possible with regard to the double bond, the present invention relates to both, the E-and Z-isomers. The bonding of vinyl is exemplified below:

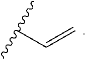 [SMILES: *C=C]

The term “cycloalkyl” as used herein denotes in each case a monocyclic cycloaliphatic radical having usually from 3 to 10 or from 3 to 6 carbon atoms, such as cyclopropyl, cyclobutyl, cyclopentyl, cyclohexyl, cycloheptyl, cyclooctyl, cyclononyl and cyclodecyl or cyclopropyl, cyclobutyl, cyclopentyl and cyclohexyl.

The term “carbocyclic” or “carbocyclyl” includes, unless otherwise indicated, in general a 3- to 9-membered, preferably a 4- to 8-membered or a 3- to 6-membered or a 5- to 7-membered, more preferably a 5- or 6-membered monocyclic ring comprising 3 to 9, preferably 4 to 8 or 3 to 6 or 5 to 7, more preferably 5 or 6 carbon atoms. The carbocycle may be saturated, partially or fully unsaturated, or aromatic, wherein saturated means that only single bonds are present, and partially or fully unsaturated means that one or more double bonds may be present in suitable positions, while the Huckel rule for aromaticity is not fulfilled, whereas aromatic means that the Hückel (4n+2) rule is fulfilled. The term “carbocycle” or “carbocyclyl”, unless otherwise indicated, may therefore cover inter alia cycloalkyl, cycloalkenyl, as well as phenyl. Preferably, the term “carbocycle” covers cycloalkyl and cycloalkenyl groups, for example cyclopropane, cyclobutane, cyclopentane and cyclohexane rings.

The term “heterocyclic” or “heterocyclyl” includes, unless otherwise indicated, in general a 3- to 9-membered, preferably a 4- to 8-membered or 5- to 7-membered, more preferably 5- or 6-membered, in particular 6-membered monocyclic ring. The heterocycle may be saturated, partially or fully unsaturated, or aromatic, wherein saturated means that only single bonds are present, and partially or fully unsaturated means that one or more double bonds may be present in suitable positions, while the Hückel rule for aromaticity is not fulfilled, whereas aromatic means that the Hückel (4n+2) rule is fulfilled. The heterocycle typically comprises one or more, e.g. 1, 2, 3, or 4, preferably 1, 2, or 3 heteroatoms selected from N, O and S as ring members, where S-atoms as ring members may be present as S, SO or SO₂. The remaining ring members are carbon atoms. In a preferred embodiment, the heterocycle is an aromatic heterocycle, preferably a 5- or 6-membered aromatic heterocycle comprising one or more, e.g. 1, 2, 3, or 4, preferably 1, 2, or 3 heteroatoms selected from N, O and S as ring members, where S-atoms as ring members may be present as S, SO or SO₂. Examples of 5- or 6-membered aromatic heterocycles include pyridyl (also referred to as pyridinyl), i.e. 2—, 3-, or 4-pyridyl, pyrimidinyl, i.e. 2—, 4- or 5-pyrimidinyl, pyrazinyl, pyridazinyl, i.e. 3- or 4-pyridazinyl, thienyl, i.e. 2- or 3-thienyl, furyl, i.e. 2- or 3-furyl, pyrrolyl, i.e. 2- or 3-pyrrolyl, oxazolyl, i.e. 2—, 3- or 5-oxazolyl, isoxazolyl, i.e. 3—, 4- or 5-isoxazolyl, thiazolyl, i.e. 2—, 3- or 5-thiazolyl, isothiazolyl, i.e. 3—, 4- or 5-isothiazolyl, pyrazolyl, i.e. 1-, 3—, 4- or 5-pyrazolyl, i.e. 1-, 2—, 4- or 5-imidazolyl, oxadiazolyl, e.g. 2- or 5-[1,3,4]oxadiazolyl, 4- or 5-(1,2,3-oxadiazol)yl, 3- or 5-(1,2,4-oxadiazol)yl, 2- or 5-(1,3,4-thiadiazol)yl, thiadiazolyl, e.g. 2- or 5-(1,3,4-thiadiazol)yl, 4- or 5-(1,2,3-thiadiazol)yl, 3- or 5-(1,2,4-thiadiazol)yl, triazolyl, e.g. 1H-, 2H- or 3H-1,2,3-triazol-4-yl, 2H-triazol-3-yl, 1H—, 2H-, or 4H-1,2,4-triazolyl and tetrazolyl, i.e. 1H- or 2H-tetrazolyl. “Hetaryls” or “heteroaryls” are covered by the term “heterocycles”. The saturated or partially or fully unsaturated heterocycles usually comprise 1, 2, 3, 4 or 5, preferably 1, 2 or 3 heteroatoms selected from N, O and S as ring members, where S-atoms as ring members may be present as S, SO or SO₂. The skilled person is aware that S, SO or SO₂ is to be understood as follows:

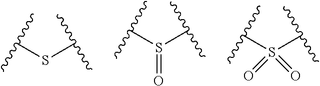

Further, a skilled person is aware that resonance structures of the oxidized forms may be possible. Saturated heterocycles include, unless otherwise indicated, in general 3- to 9-membered, preferably 4- to 8-membered or 5- to 7-membered, more preferably 5- or 6-membered monocyclic rings comprising 3 to 9, preferably 4 to 8 or 5 to 7, more preferably 5 or 6 atoms comprising at least one heteroatom, such as pyrrolidine, tetrahydrothiophene, tetrahydrofuran, piperidine, tetrahydropyran, dioxane, morpholine or piperazine.

As used herein the term “carbobicyclic” or “carbobicyclyl” includes in general 6 to 14-membered, preferably 7- to 12-membered or 7- to 10-membered bicyclic rings comprising 6 to 14, preferably 7 to 12 or 7 to 10 carbon atoms. The carbobicycle may be saturated, partially or fully unsaturated, or aromatic, wherein saturated means that only single bonds are present, and partially or fully unsaturated means that one or more double bonds may be present in suitable positions, while the Huckel rule for aromaticity is not fulfilled, whereas aromatic means that the Hückel (4n+2) rule is fulfilled. Preferably, the term “aromatic” in connection with the carbobicyclic ring means that both rings of the bicyclic moiety are aromatic, so that, e.g., 8 It electrons are present in case of a 10-membered aromatic carbobicyclic ring. The term “carbobicyclic” or “carbobicyclyl”, unless otherwise indicated, may therefore cover inter alia bicycloalkyl, bicycloalkenyl, as well as bicyclic aromatic groups, for example bicyclohexane (decalin), bicycloheptane (such as norbornane), bicyclooctane (such as bicyclo[2.2.2]octane, bicyclo[3.2.1]octane or bicyclo[4.2.0]octane), bicyclononane (such as bicyclo[3.3.1]nonane or bicyclo[4.3.0]nonane), bicyclodecane (such as bicyclo[4.4.0]decane), bicycloundecane (such as bicyclo[3.3.3]undecane), norbornene, naphthalene and the like. Preferably, the carbobicycle is a fused carbobicycle, which is preferably aromatic, for example naphthalene.

The term “heterobicyclic” or “heterobicyclyl” includes, unless otherwise indicated, in general 6 to 14-membered, preferably 7- to 12-membered or 7- to 10-membered bicyclic rings. The heterobicycle may be saturated, partially or fully unsaturated, or aromatic, wherein saturated means that only single bonds are present, and partially or fully unsaturated means that one or more double bonds may be present in suitable positions, while the Hückel rule for aromaticity is not fulfilled, whereas aromatic means that the Hückel (4n+2) rule is fulfilled. In principal, for being “aromatic”, it is sufficient if one of the two rings of the bicyclic moieties is aromatic, while the other is non-aromatic. However, it is preferred in connection with the term “aromatic” that both rings of the bicyclic moiety are aromatic, so that, e.g., 8 It electrons are present in case of a 9- or 10-membered aromatic heterobicyclic ring. The heterobicycle typically comprises one or more, e.g. 1, 2, 3, or 4, preferably 1, 2, or 3 heteroatoms selected from N, O and S as ring members, where S-atoms as ring members may be present as S, SO or SO₂. The remaining ring members are carbon atoms. Examples of heterobicycles include benzofuranyl, benzothienyl, indolyl, indazolyl, benzimidazolyl, benzoxathiazolyl, benzoxadiazolyl, benzothiadiazolyl, benzoxazinyl, quinolinyl, isoquinolinyl, purinyl, or quinuclidine and the like. Preferred heterobicycles according to the invention are aromatic heterobicycles.

As used herein, the terms “carbocyclylalkyl”, “heterocyclylalkyl”, and “cyclopropylalkyl” as well as the terms “NRR-alkyl”, “phenyl-alkyl, benzophenone-alkyl, phenoxybenzene-alkyl, N-methyl-diphenylamine-alkyl, diphenylsulfide-Calkyl, or naphthalene-alkyl” and the like refer to the corresponding groups, which are bonded to the remainder of the molecule via an alkyl, preferably via a C₁-C₄-alkyl group. Examples include but are not limited to benzyl(i.e. phenylmethyl), cyclohexylmethyl, pyridinylmethyl, and piperidinomethyl.

As used herein the term “spiro-heterocyclyl” refers to a polycyclic heterocyclyl having usually from 7 to 10 atoms. The atoms may be carbon or heteroatoms, wherein the spiro-heterocycle comprises at least one heteroatom, preferably 1 to 3 heteroatoms, more preferably 1 or 2 heteroatoms. The remaining atoms of the spiro-heterocycle are carbon atoms. The polycycle is preferably a bicycle, preferably a heterobicycle having usually from 7 to 10 atoms. The cycles of the polycycle, preferably the two cycles of the heterobicycle are attached to each other over one atom only, which is referred to as the “spiro-atom”, wherein said spiro-atom may be a carbon or heteroatom. The cycles of the polycyclic ring may be the same or different.

As used in the specification and the claims, the singular forms of “a” and “an” also include the corresponding plurals unless the context clearly dictates otherwise. The same applies for plural forms used herein, which also include the singular forms unless the context clearly dictates otherwise.

The terms “about” and “approximately” in the context of the present invention denotes an interval of accuracy that a person skilled in the art will understand to still ensure the technical effect of the feature in question. The term typically indicates a deviation from the indicated numerical value of +10% and preferably +5%.

It needs to be understood that the term “comprising” is not limiting. For the purposes of the present invention, the term “consisting of” is considered to be a preferred embodiment of the term “comprising of”. If hereinafter a group is defined to comprise at least a certain number of embodiments, this is also meant to encompass a group, which preferably consists of these embodiments only.

The term “pharmaceutically acceptable excipient” as used herein refers to compounds commonly comprised in pharmaceutical compositions, which are known to the skilled person. Examples of suitable excipients are exemplary listed below. Typically, a pharmaceutically acceptable excipient can be defined as being pharmaceutically inactive.

The term “treatment” is to be understood as also including the option of “prophylaxis”. Thus, whenever reference is made herein to a “treatment” or “treating”, this is to be understood as “treatment and/or prophylaxis” or “treating and/or preventing”.

The term “seven-beta-strand family of histone methyltransferases” refers to the respective family of enzymes. Presently, this family comprises DOT1L and KMT9.

The term “KTM9” means the heterodimer composed of KMT9 alpha and KMT9beta. The term “KMT9alpha” as used herein refers to the protein “N-6 adenine-specific DNA methyltransferase 1” [Homo sapiens (human)], with the underlying Gene ID: 29104 (updated on 11Sep. 2019, database: https://www.ncbi.nlm.nih.gov/gene). “N6AMT1” or “KMT9alpha” is the corresponding gene. Other names for KMT9alpha are C₂₁orf127, Hemk2, Mtq2, N6amt1, PrmC or PRED28. The sequence of the KMT9alpha protein (isoform 1 [Homo sapiens]) is depicted in SEQ ID NO: 1. The term “KMT9beta” as used herein refers to the protein “tRNA methyltransferase subunit11-2” [Homo sapiens (human)] with the underlying Gene ID: 51504 (updated on 11Sep. 2019, database: https://www.ncbi.nlm.nih.gov/gene). “TRMT112” or “KMT9beta” is the corresponding gene. The sequence of the KMT9beta protein (isoform 2 [Homo sapiens]) is depicted in SEQ ID NO: 2.

### Description of Pharmaceutical Compositions According to the Present Invention

A pharmaceutical composition according to the present invention may be formulated for oral, buccal, nasal, rectal, topical, transdermal, or parenteral application. Preferred non-parenteral routes include mucosal (e.g., oral, vaginal, nasal, cervical, etc.) routes, of which the oral application may be preferred. Preferred parenteral routes include but, are not limited to, one or more of subcutaneous, intravenous, intra-muscular, intraarterial, intradermal, intrathecal, and epidural administrations. Preferred administration is by subcutaneous, intratumoral or peritumoral routes. Particularly preferred is intratumoral administration. The compound according to formula (I) should be applied in pharmaceutically effective amounts, for example in the amounts as set out herein below.

A pharmaceutical composition of the present invention may also be designated as formulation or dosage form. A compound of formula (I) may also be designated in the following as (pharmaceutically) active agent, active ingredient, or active compound.

Pharmaceutical compositions may be solid or liquid dosage forms or may have an intermediate, e.g. gel-like character depending inter alia on the route of administration.

In general, the inventive dosage forms can comprise various pharmaceutically acceptable excipients, which will be selected depending on which functionality is to be achieved for the dosage form. A “pharmaceutically acceptable excipient” in the meaning of the present invention can be any substance used for the preparation of pharmaceutical dosage forms, including coating materials, film-forming materials, fillers, disintegrating agents, release-modifying materials, carrier materials, diluents, binding agents, and other adjuvants. Typical pharmaceutically acceptable excipients include substances like sucrose, mannitol, sorbitol, starch and starch derivatives, lactose, and lubricating agents such as magnesium stearate, disintegrants, and buffering agents.

The term “carrier” denotes pharmaceutically acceptable organic or inorganic carrier substances with which the active ingredient is combined to facilitate the application. Suitable pharmaceutically acceptable carriers include, for instance, water, aqueous salt solutions, alcohols, oils, preferably vegetable oils, propylene glycol, polyoxyethelene sorbitans, polyethylene-polypropylene block co-polymers such as poloxamer 188 or poloxamer 407, polyethylene glycols such as polyethylene glycol 200, 300, 400, 600, etc., gelatin, lactose, amylose, magnesium stearate, surfactants, perfume oil, fatty acid monoglycerides, diglycerides and triglycerides, polyoxyethylated medium or long chain fatty acids such as ricinoleic acid, and polyoxyethylated fatty acid mono-, di-, and triglycerides such as capric or caprilic acids, petroethral fatty acid esters, hydroxymethyl celluloses such as hydroxymethyl, hydroxyethyl, hydroxypropyl, hydroxypropyl acetate succinate, polyvinylpyrrolidone, crosspovidone, and the like.

The pharmaceutical compositions can be sterile and, if desired, mixed with auxiliary agents, like lubricants, preservatives, stabilizers, wetting agents, emulsifiers, salts for influencing osmotic pressure, buffers, colorings, flavoring and/or aromatic substances and the like which do not deleteriously react with the active compound.

If liquid dosage forms are considered for the present invention, these can include pharmaceutically acceptable emulsions, solutions, suspensions, and syrups containing inert diluents commonly used in the art such as water. These dosage forms may contain e.g. microcrystalline cellulose for imparting bulk, alginic acid or sodium alginate as a suspending agent, methylcellulose as a viscosity enhancer and sweeteners/flavoring agents.

For parenteral application, particularly suitable vehicles consist of solutions, preferably oily or aqueous solutions, as well as suspensions, emulsions, or implants. Pharmaceutical formulations for parenteral administration are particularly preferred and include aqueous solutions of the compounds of formula (I) in water-soluble form. Additionally, suspensions of the compounds of formula (I) may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances, which increase the viscosity of the suspension, such as sodium carboxymethyl cellulose, sorbitol, or dextran.

In one preferred embodiment, dosage forms are injectable preparations of a compound of formula (I). Thus, sterile injectable aqueous or oleaginous suspensions can for example be formulated according to the known art using suitable dispersing agents, wetting agents and/or suspending agents. A sterile injectable preparation can also be a sterile injectable solution or suspension in a non-toxic parenterally acceptable diluent or solvent. Among the acceptable vehicles and solvents that can be used are water and isotonic sodium chloride solution. Sterile oils are also conventionally used as solvent or suspending medium. Preferred applications for injectable preparations comprising the compounds of the present invention are intravenous, intratumoral and peritumoral administration.

Suppositories for rectal administration of a compound of formula (I) can be prepared by e.g. mixing the compound with a suitable non-irritating excipient such as cocoa butter, synthetic triglycerides and polyethylene glycols which are solid at room temperature but liquid at rectal temperature such that they will melt in the rectum and release the compound according to formula (I) from said suppositories.

For administration by inhalation, the compounds according to the present invention may be conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

In one preferred embodiment the pharmaceutical composition is an oral dosage form. Oral dosage forms may be liquid or solid and include e.g. tablets, troches, pills, capsules, powders, effervescent formulations, dragees, and granules. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. The oral dosage forms may be formulated to ensure an immediate release of the compound of formula (I) or a sustained release of the compound of formula (I).

A solid dosage form may comprise a film coating. For example, the inventive dosage form may be in the form of a so-called film tablet. A capsule of the invention may be a two-piece hard gelatin capsule, a two-piece hydroxypropylmethylcellulose capsule, a two-piece capsule made of vegetable or plant-based cellulose or a two-piece capsule made of polysaccharide.

The dosage form according to the invention may be formulated for topical application. Suitable pharmaceutical application forms for such an application may be a topical nasal spray, sublingual administration forms and controlled and/or sustained release skin patches. For buccal administration, the compositions may take the form of tablets or lozenges formulated in conventional manner.

The compositions may conveniently be presented in unit dosage forms and may be prepared by any of the methods well known in the art of pharmacy. The methods can include the step of bringing the compounds into association with a carrier, which constitutes one or more accessory ingredients. In general, the compositions are prepared by uniformly and intimately bringing the compounds into association with a liquid carrier, a finely divided solid carrier, or both, and then, if necessary, shaping the product. Liquid dose units are vials or ampoules. Solid dose units are tablets, capsules and suppositories.

As regards human patients, the compound of formula (I) may be administered to a patient in an amount of about 0.001 mg to about 5000 mg per day, preferably of about 0.01 mg to about 1000 mg per day, more preferably of about 0.05 mg to about 250 mg per day, which is the effective amount. The phrase “effective amount” means an amount of compound that, when administered to a mammal in need (i.e. a patient in need) of such treatment, is sufficient to treat or prevent a particular disease or condition.

In one embodiment, the pharmaceutical composition may contain the compound of formula (I) in the form of a prodrug. A prodrug is generally any compound, which is converted under physiological conditions or by solvolysis to a more potent compound. A prodrug may be inactive or only slightly active prior to administration but may be converted to an active compound of the invention in vivo.

It is to be understood that the use of either a prodrug or a compound that has shown to have strong in vitro inhibitory capacity depends on the pharmaceutical composition and the route of administration that is used. If a pharmaceutical composition is used that includes a delivery system of an active agent into an intact cell, one would be inclined to use a compound with a strong in vitro inhibitory capacity, while rather a compound assumed to be a prodrug would be used if the pharmaceutical formulation rather delivers the compound to the cell membrane of an intact cell.

In one embodiment, the present invention relates to a pharmaceutical composition comprising a compound according to formula (I) as defined above with regard to the second aspect of the present invention and optionally a pharmaceutically acceptable carrier, diluent, or escipient as defined above for use in the treatment of castration resistant prostate cancer.

Indications for which the Compounds of the Present Invention May be Used

The compounds according to the present invention, in particular the compounds of formula (I) according to the first, third and fourth aspect of the present invention or the pharmaceutical composition comprising the same are suitable for use in medicine. In particular, the compounds according to formula (I) of the first, third and fourth aspect of the present invention are suitable for use in the treatment of cancer.

In one embodiment, the compound of formula (I) according to the first, third and fourth aspect of the present invention or a pharmaceutical composition comprising the same is for use in the treatment of cancer selected from the group consisting of prostate cancer, breast cancer, ovarian cancer, colon cancer, glioblastoma, lung cancer, neuroblastoma, osteosarcoma, liposarcoma, colorectal cancer, rectal adenocarcinoma, mesothelioma, endometrium adenocarcinoma, leukemia, erythroleukemia, medulloblastoma, astrocytoma, Ewing sarcoma, myelodysplastic syndrome (MDS), diffuse large B-cell lymphoma, myelogenic leukemia, myeloid leukemia, acute monocytic leukemia, gallbladder carcinoma, cecum adenocarcinoma, gastric adenocarcinoma, stomach adenocarcinoma, renal cell carcinoma, bladder carcinoma, melanoma, cervical squamous cell carcinoma, pancreatic carcinoma, chondrosarcoma, duodenal adenocarcinoma, rhabdomyosarcoma, hepatocellular carcinoma and uterine adenocarcinoma.

In a preferred embodiment of the present invention, the cancer is selected from the group consisting of prostate cancer, breast cancer, ovarian cancer, colon cancer, glioblastoma, lung cancer, neuroblastoma, colorectal cancer and bladder carcinoma.

In a particularly preferred embodiment of the present invention, the compound of formula (I) according to the first, third and fourth aspect of the present invention or a pharmaceutical composition comprising the same is for use in the treatment of prostate cancer, preferably castration resistant prostate cancer.

With regard to the above embodiments it is noted that the prostate cancer may be hormone-dependent prostate cancer or castration-resistant prostate cancer, wherein the castration resistant prostate cancer may be further resistant to enzalutamide. Preferably, the prostate cancer as mentioned above is castration resistant prostate cancer.

Furthermore, in connection with the above embodiments it is to be understood that the lung cancer may be non-small cell lung cancer or small cell lung cancer.

In another embodiment of the present invention the compounds according to the present invention, in particular the compounds of formula (I) according to the second aspect of the present invention are for use in the treatment of prostate cancer.

In a particularly preferred embodiment of the present invention, said prostate cancer is a castration resistant prostate cancer. In this connection it is to be understood that the castration resistant prostate cancer may be further resistant to enzalutamide.

The present invention is further illustrated by the following examples.

### EXAMPLES

### 1. Synthesis of Compounds

List of abbreviations

| | |
|---|---|
| Ac | acetyl |
| ACN | acetonitrile |
| AcOH | acetic acid |
| AcOEt | ethyl acetate |
| BINAP | (2,2′-bis(diphenylphosphino)-1,1′-binaphthyl) |
| C18 | C18 stationary phase column |
| DEAD | diethyl azodicarboxylate |
| DIAD | diisopropyl azodicarboxylate |
| DIEA | N,N-diisopropylethylamine |
| DCE | dichloromethane |
| DCM | dichloromethane |
| DMAP | 4-dimethylaminopyridine |
| DMF | N,N-dimethylformamide |
| DMP | Dess-Martin periodinane |
| DMSO | dimethyl sulfoxide |
| dba | dibenzylideneacetone |
| dppf | bis(diphenylphosphino)ferrocen |
| dtbpf | 1,1′-bis(di-tert-butylphosphino)ferrocene |
| dr | diastereomeric ratio |
| ES | electrospray ionization |
| EtOH | ethanol |
| FA | formic acid |
| h | hours |
| HATU | 1-[Bis(dimethylamino)methylene]-1H-1,2,3- |
| | triazolo[4,5-b]pyridinium |
| | 3-oxide hexafluorophosphate |
| HPLC | high performance liquid chromatography |
| LCMS | liquid chromatography-mass spectrometry |
| M | Molar |
| m-CPBA | meta-chloroperoxybenzoic acid |
| MeOH | methanol |
| MH: | megahertz |
| min | minutes |
| mL | millilitres |
| mM | millimole |
| MTBE | methyl tert-butyl ether |
| Ms | mesylate |
| MSA | methanesulfonic acid |
| NBS | N-bromosuccinimide |
| NIS | N-iodosuccinimide |
| NMR | nuclear magnetic resonance |
| NMP | N-methyl-2-pyrrolidone |
| PCy₃ | tricyclohexylphosphine |
| Prep-HPLC | preparative-scale high performance liquid |
| | chromatography |
| Prep-TLC | preparative thin layer chromatography |
| P(t-Bu)₃-PdCl-2nd G | (chloro[(tri-tert-butylphosphine)-2-(2- |
| | aminobiphenyl)] palladium(II)) |
| SEM | trimethylsilylethoxymethyl |
| THF | tetrahydrofuran |
| T_{R} | retention time |
| Ts | toluenesulfonyl |
| TsOH | p-toluenesulfonic acid |
| SFC | supercritical fluid chromatography |
| TEA or Et₃N | triethylamine |
| TFA | trifluoroacetic acid |
| UV | ultraviolet |
| PE | petroleum ether |
| EtOAC | ethylacetate |
| DIPEA | diisopropylethylamine |
| EDCI | 1-ethyl-3-(3-dimethylaminopropyl)carbodiimide |
| NaBH4 | sodium borohydride |
| STAB | sodium trisacetoxyborohydride |
| NH₄Cl | ammonium chloride |
| DMP | Dess-Martin-periodinane |

Due to the respective synthetic procedure, the compounds according to the following examples may be provided as the corresponding salt thereof, such as e.g., a trifluoroacetate salt.

### Materials & Methods

All reactions were carried out in glassware under inert (nitrogen) atmosphere. All used chemicals and reagents were purchased from commercial sources and were used without further purification. Solvents were freshly purified by distillation/drying over molecular sieves following the instructions from the Purification Book. Particularly mentioned anhydrous/dry solvents were purchased from Acros organics. Reactions were monitored by thin-layer chromatography (TLC) performed with Merck alumina plates coated with silica gel 60 F254, silica gel 60RP-18 F254s or silica gel 60 NH₂ F254S (layer thickness: 0.2 mm) and analyzed under UV light (254 nm and 365 nm) or revealed using KMnO₄, Bromocresol green, ninhydrin, phosphomolybdic acid or 2,4-dinitrophenylhydrazine (2,4-DNPH) as staining agent. The composition of the mobile phase was adjusted to the compound properties. Yields were not optimized. Flash column chromatography was performed on a Biotage® Isolera Prime/One purification system using 40-60 μm pre-packed silica gel columns from Biotage®, HP-spherical 50 μm pre-packed silica gel columns from Interchim (Jumbo Pack), Sfär Silica D 60 μm, Sfär KP amino D 50 μm or Sfär Silica HC D 20 μm pre-packed silica gel columns from Biotage®. NMR spectroscopy and mass spectrometry were used for product identification. NMR spectra were acquired on a BRUKER Avance 400 spectrometer (400 MHz and 100.6 MHz for ¹H and ¹³C respectively), at a temperature of 303 K unless specified using DMSO-d, as solvent. Chemical shifts (δ) are reported in ppm, multiplicity abbreviations are as follows: bs=broad singlet, s=singlet, d=doublet, dd=doublet of doublets, dt=doublet of triplets, t=triplet, td=triplet of doublets, q=quartet, m=multiplet, coupling constant (J) are expressed in Hz. The ¹H assignment resulted from COSY experiments. Mass spectra were recorded on an Advion expression CMS using an ASAP®(Atmospheric Solids Analysis Probe; aka APCI: Atmospheric Pressure Chemical Ionization) as ion source, on a Thermo Scientific Exactive mass spectrometer using electrospray ionization (ESI) as ion source or HR-MS were obtained on a THERMO SCIENTIFIC Advantage. HPLC analysis was performed to determine the purity of all final compounds on an Agilent Technologies 1260 Infinity II system using diode array detector (DAD) UV detection at either 230, 248, 254, 260 & 280 nm or at 210 & 260 nm. 2 methods were used: Method A: Phenomenex Kinetex® 5 μm XB-C18 100 Å 250×4.6 mm column and eluent A was H₂O containing 0.05% trifluoracetic acid (TFA) and eluent B was CH₃CN containing 0.05% TFA. Linear gradient conditions were as follows: 0-4 min: 90:10 (A/B); 4-29 min: 90:0-+100 (A/B); 29-31 min: 0:100; (A/B); 31-31.5 min: 90:10 (A/B); 31.5-40 min: 90:10 (A/B) with a flowrate of 1.00 mL.min-1.

Method A XBridge: XBridge® Shield RP18 5 μm XB-C18 100 Å 150×4.6 mm column and eluent A was H₂O containing 0.05% trifluoracetic acid (TFA) and eluent B was CH₃CN containing 0.05% TFA. Linear gradient conditions were as follows: 0-4 min: 90:10 (A/B); 4-19 min: 90:0-+100 (A/B); 19-21 min: 0:100; (A/B); 21-31.5 min: 90:10 (A/B); 31.5-25 min: 90:10 (A/B) with a flowrate of 1.00 mL.min⁻¹.

Method B: Phenomenex Kinetex® 5 μm XB-C18 100 Å 250×4.6 mm column and eluent A was H₂O containing 0.05% trifluoracetic acid (TFA) and eluent B was CH₃CN containing 0.05% TFA. Linear gradient conditions were as follows: 0-1 min: 100:0 (A/B); 1-9 min: 60:40 (A/B); 9-11 min: 5:95; (A/B); 11-13 min: 5:95 (A/B); 13-14 min: 100:0 (A/B); 14-16 min: 100:0 (A/B) with a flowrate of 0.95 mL.min⁻¹.

Method B XBridge: XBridge® Shield RP18 5 μm XB-C18 100 Å 150×4.6 mm column and eluent A was H₂O containing 0.05% trifluoracetic acid (TFA) and eluent B was CH₃CN containing 0.05% TFA. Linear gradient conditions were as follows: 0-1 min: 100:0 (A/B); 1-9 min: 60:40 (A/B); 9-11 min: 5:95; (A/B); 11-13 min: 5:95 (A/B); 13-14 min: 100:0 (A/B); 14-16 min: 100:0 (A/B) with a flowrate of 0.95 mL.min-1.

### HPLC Purification Methods:

Method C: Prep-HPLC was performed at conditions: (Flash: Welchrom C18, 150×20 mm); Wavelength 220 nm; Mobile phase: A MeCN (0.1% TFA); B water (0.1% TFA); Flow rate: 25 mL/min; Injection volume: 2 mL; Run time: 30 min; Equilibration: 5 min.

Method D: Phenomenex Kinetex® 5u XB-C18 100 Å 250×21.2 mm column and eluent A was H₂O containing 0.05% trifluoracetic acid (TFA) and eluent B was CH₃CN containing 0.05% TFA. Linear gradient conditions were as follows: 0-4 min: 90:10 (A/B); 4-29 min: 90:0-+100 (A/B); 29-31 min: 0:100; (A/B); 31-31.5 min: 90:10 (A/B); 31.5-40 min: 90:10 (A/B) with a flowrate of 22.00 mL.min-1.

Method E: Phenomenex Kinetex® 5u XB-C18 100 Å 250×21.2 mm column and eluent A was H₂O containing 0.05% trifluoracetic acid (TFA) and eluent B was CH₃CN containing 0.05% TFA. Linear gradient conditions were as follows: 0-1 min: 100:0 (A/B); 1-9 min: 100:0-+60:40 (A/B); 9-11 min: 60:40-+5:95; (A/B); 11-13 min: 5:95 (A/B); 13-14 min: 5:95-+100:0 (A/B); 14-20 min: 100:0 (A/B) with a flowrate of 20.20 mL.min-1.

Method F: Chiral HPLC: column: chiralpak IC, 250 mm×4.6 mm, 5 μm; mobile phase: hexane/ethanol/diethylamine=70:30:0.3; Flow rate: 1.0 mL/min; Wavelength: 230 nm; T=30° C.

Method G: XBridge® Prep Shield RP 18 5 μm OBD™ 19×150 mm column and eluent A was H₂O containing 0.05% TFA and eluent B was CH₃CN containing 0.05% TFA. Linear gradient conditions were as follows: 0-4 min: 90:10 (A/B); 4-19 min: 90:10->0:100 (A/B); 19-21 min: 0:100 (A/B); 21-21.5 min: 0:100->90:10 (A/B); 21.5-25 min: 90:10 (A/B) with a flowrate of 1.00 mL/min (procedure B).

### General Procedures for Final Compounds

Reductive amination general procedure A: An aldehyde 3, 7, 32, 42, and 49 (1.00 eq.) was dissolved in dry MeOH (0.10 M based on the aldehyde). Then a solution of amine 138, 144, and 150 (1.00 eq.) in dry MeOH (0.10 M based on amine) was added and stirred at ambient temperature for 72 h. The solution was cooled down to 0° C. in an ice-bath before NaBH₄ (1.5 eq.) was added portion wise. The ice-bath was removed, and the solution was stirred for 6 h at ambient temperature. Afterwards, water was added, and the aqueous phase was extracted 3 times with EtOAc. The combined organic layers were washed with brine, dried over sodium sulfate, and concentrated under reduced pressure. The crude product was purified over silica eluting with CH₂Cl₂/MeOH (mostly 0-10%) to afford the secondary amine 286, 288, 290, 308, 310, 320 and 322 as yellowish foams to colorless foams.
Reductive amination general procedure B: For the secondary amines: A solution of aldehyde 13, 19, and 40 (1.20 eq.) and amine 150 (1.00 eq.) in MeOH (0.02 M based on 150) was stirred at room temperature for 20 min. NaBH₃CN (3.00 eq.) and AcOH (0.10 eq.) was added. The solution was stirred at room temperature for 4 hrs. The resulting mixture was diluted with EtOAc, washed with H₂O. The organic phase was dried over Na₂SO₄ and concentrated. The crude was purified by prep-HPLC using method C with varying buffer system (mentioned in the compound description) to give the pure compounds 361, 363, and 365 For the tertiary amines: To the reaction mixture was directly added either acetaldehyde, acetone, cyclopropanecarbalydehyde (1.20 eq.) and the mixture was stirred at room temperature for 12 hrs. The target was found by LC-MS. The reaction solution was quenched with several drops of water and concentrated to dryness. The residue was purified by prep-HPLC using method C with varying buffer system (mentioned in the compound description) to give the pure compounds 369, 371, and 373.

Acylation/urea synthesis general procedure C: To an ice-cooled solution of Fmoc-protected amine 111 (1.00 eq.) in a mixture of CH₂Cl₂ and Et₃N (0.03 M based on 111) was added the corresponding isocyanate or the acylchloride (1.00 eq.). The resulted solution was stirred for 24 h at ambient temperature. The reaction mixture was concentrated over vacuum to complete dryness. The obtained residue was purified by flash chromatography eluting CH₂Cl₂/MeOH (mostly 0-15%) to afford the penultimate products 312 and 314 yellowish foam.
Reductive amination general procedure D: To a solution of aldehyde 3, 27, 29, 30, 42, 59, 60, 63, 66, and 67 (1.10 eq.) in dry DCE (0.02 M based on aldeyhde) was added a solution of amine 127, 150, 156, 164, 167, 172, and 175 (0.02 M based on amine) under nitrogen atmosphere. The solution was stirred for 30 min at ambient temperature. Then, NaBH (OAc)₃ (1.50 eq.) was added portion wise under nitrogen. The resulted mixture was tired for 24 h at ambient temperature. HPLC analytics showed complete consumption of the amine. At this point, the reaction was quenched by addition of 1 M K2CO3 solution to reach pH 8-9. Then, CH₂Cl₂ was added, and the organic layer was separated. The aqueous phase was further extracted with CH₂Cl₂ (2 times). The combined organic layers were washed with brine, dried over sodium sulfate, and concentrated. The crude product was purified over silica CH₂Cl₂/MeOH (mostly 0-7%) to afford the product 310, 316, 318, 327, 329, 331, 333, 335, 337, 339, 341, 343, 378 and 380 as colorless foam.

Peptidic coupling general procedure E: The acid 133 (1.20 eq.) was suspended in DMF (0.20 M based on amine). The suspension was cooled down with an ice-bath before EDCl (1.50 eq.) was portion wise added. The mixture was stirred for 10 min at 0° C. and then allowed to warm up to rt. Then, a solution of amine 69, 71, 91, 98, 102, 103, 107, and 109 in CH₂Cl₂ (0.2 M based on amine), DIPEA, and DMAP were added. The mixture was stirred overnight at ambient temperature. Then, the organic solvent was evaporated by rotatory evaporation. The obtained residue was subjected to silica gel column chromatography eluting with CH₂Cl₂/MeOH (mostly 0-5%) to afford the penultimate amides 296, 298, 300, 302, 304, 306, 324, 683 and 687 as yellowish foams. Peptidic coupling general procedure F: A solution of acid 135 (1.00 eq), amine tert-butyl 3-(aminomethyl) piperidine-1-carboxylate, tert-butyl 2-(aminomethyl) piperidine-1-carboxylate 1-Boc-4-(aminomethyl) piperidine, 75, 77, 78, 79, 80, and 98 (2.00 eq.) and DIPEA (3.00 eq.) in DMF (0.10 M based on acid) was stirred at r. t. for 10 min. HATU (1.50 eq.) was then added, and the reaction mixture was stirred at r. t. overnight. The reaction solution was directly purified by prep-HPLC(TFA or NH₄OAc buffer) to give the desired compounds 345, 347, 349, 351, 352, 353, 354, 355, and 356 as white foams (TFA salt or free base).

CuAAC general procedure G: The alkyne 181 and 182 (1.00 eq.), THPTA (0.20 eq.), and CuBr (0.20 eq.) in a tBuOH/H₂O (2:1; 0.01 M based on alkyne). Then, 1-azido-2-methoxyethane (1.10 eq.) was added to the reaction mixture at ambient temperature. The mixture was stirred for 24 h at rt. The organic solvent was evaporated and the aqueous phase was extracted with CH₂Cl₂. The combine organic layers were washed with brine, dried, and concentrated. The obtained residue was purified over silica (CH₂Cl₂/MeOH; mostly 0-10%) to afford the pure products 292 and 294 as colorless solid.
Rh-catalyzed 1,4-addition general procedure H: To a heat-dried three-necked round bottom flask equipped with a stirring bar and air condenser was charged with (3αR,6αR)-2,2-dimethyl-3a,6a-dihydro-4H-cyclopenta[d][1,3]dioxol-4-one (1.00 eq.), [Cp*RhCl₂]₂ (0.01 eq.), KOH (0.2 eq.), and a corresponding aryl or alkenyl pinacol ester (1.50 eq.) under nitrogen atmosphere, Then, 1,4-dioxane (0.20 M) was added and the resulted mixture was degassed. Degassed water was then added, and the mixture was placed into a pre-heated heating plate (50° C.). The reaction mixture was heated to 80° C. and stirred for 3 to 7 h upon complete consumption of the carbasugar mimic. TLC was used for monitoring the reaction progress. Then, the reaction was cooled down to ambient temperature and was diluted with water. The aqueous phase was extracted three times with EtOAc and the combined organic layers were washed with brine, dried over sodium sulfate, and concentrated under reduced pressure. The obtained crude product was purified by flash chromatography to obtain the desired products 213, 214, 215, 216, 217, 218, 219, 220, 221, 222, 223, 224.

Ketone reduction general procedure I: Compounds 213, 214, 215, 216, 217, 218, 219, 220, 221, 222, 223, 224 (1.00 eq.) were dissolved in anhydrous MeOH (0.20 M) and cooled down to 0° C. Then, NaBH₄ (1.50 eq.) was added portion wise. The reaction mixture was stirred for 1 h at 0° C. (until the bubbling stopped). Upon full conversion indicated by TLC, Cold water was added. The aqueous phase was extracted five times with CH₂Cl₂. The combined organic layers were dried over sodium sulfate and concentrated under reduced pressure. The obtained residue was purified with silica gel chromatography to afford the pure products 227, 228, 229, 230, 231, 232, 233, 234, 235, 236, 237, 238, 239. 240.

Nucleophilic substitution general procedure J: The alcohol 227 & 228 (1.00 eq.) was dissolved in dry CH₂Cl₂ (0.10 M). Then, dry pyridine (3.00 eq.) was added and cooled down to 0° C. To the cooled solution Tf₂O (2.00 eq.) was added dropwise. The reaction mixture was stirred for 1 h at 0° C. Then, the reaction mixture was quenched with cold water and extracted with CH₂Cl₂. The combined organic layers were dried over sodium sulfate and concentrated to complete dryness.

Nucleobase (1.20 eq.) was dissolved in dry DMF (0.08 M) and cooled down to 0° C. before NaH (2.00 eq.) was added portion wise. The resulted mixture was stirred for 15 min at ambient temperature before a corresponding triflate solution (1.00 eq.) in DMF was added dropwise. The mixture was stirred for 17 h at ambient temperature. Then, water was added carefully, and the resulted mixture was extracted with EtOAc. The combined organic layers were washed extensively with brine, dried over sodium sulfate, and concentrated under reduced pressure. The crude products were purified over silica to afford the target compounds 244, 245, 246.

Mitsunobu type glycosylation general procedure K: To a cooled solution of compounds 229, 230, 231, 232, 233 (1.00 eq.) in dry THF was added PPh₃ (2.00 eq.) followed by dropwise addition of DIAD (1.80 eq.) under nitrogen atmosphere. The resulted mixture was stirred for 30 min at 0° C.

Then, corresponding nucleobase (1.40 eq.) was added at 0° C. The reaction mixture was stirred for 17 h at ambient temperature. After 17 h, the reaction was diluted with saturated bicarbonate solution and extracted with EtOAc. The combined organic layers were washed with brine, dried over sodium sulfate, and concentrated under reduced pressure. The obtained residue was purified by flash chromatography to afford the target compounds 247, 248, 249, 250, 251, 252, 253 Mitsunobu type glycosylation general procedure L: A heat dried three-necked round bottom flask equipped with stirring bar, thermometer, and air condenser was charged with alcohol 192, 197, 234, 235, 236 (1.50 eq.) under nitrogen atmosphere. Then, dry toluene (0.50 M) was added and cooled down to 0° C. To the solution PPh₃ (2.00 eq.) and nucleobase (1.00 eq.) was added and cooled down to 0° C. To the cooled solution a solution of DBAD (1.10 M, 2.00 eq.) in dry toluene was added dropwise under nitrogen atmosphere. The mixture was stirred 10 min at 0° C. before heated to 60° C. for 17 h. The resulting mixture was allowed to cool to room temperature and was concentrated under reduced pressure. The crude residue was purified by silica gel column chromatography to afford the title compounds 193, 254, 255, 256, 257, 259. Aromatic substitution general procedure M: A pressure flask was charged with compound 193, 244, 245, 246, 247, 248, 249, 250, 251, 252, 253, 254, 255, 256 (1.00 eq.). Then, a 2:1 mixture of ammonia/1,4-dioxane ( ) was added, and the resulted mixture was heated to 100° C. and stirred upon full consumption. After 24 h, the reaction was allowed to cool down to rt and concentrated to complete dryness. The crude products were purified over silica gel chromatography to afford the target compounds 264, 265, 266, 267, 268, 269, 270, 271, 272, 273, 274, 275, 276. Deprotection general procedure N: A solution of 358, 361, 363, 365, 369, 371, and 373 (0.01 M) in DCM/TFA (1:1) was stirred at room temperature for 16 hrs. The mixture was concentrated to dryness and the residue was purified by prep-HPLC to afford the products 357, 360, 362, 364, 366, 367, 368, 370, 372, 374, and 375 as white foam.

Deprotection general procedure O: Secondary amines 316, 318, 333, 335, 337, 339, 341, 343, 378, 380, and 382were dissolved (0.02 M) in pure TFA and stirred at 50° C. for 24 h. The solvent was evaporated by rotatory evaporation at 45° C. The crude products were purified by preparative HPLC according to method C to afford the desired products 317, 319, 334, 336, 338, 340, 342, 344, 379, and 381 as colorless foams (2 TFA).

Deprotection general procedure P: A solution of amides 345 & 347 were dissolved (0.02 M) in MeOH/HCl (1:1) and were stirred at r. t. for 1 hr, followed by concentration to afford the desired compounds 346, 348 and 350 as a white solid (TFA salt or free base).

Deprotection general procedure Q: Secondary amines and amides 194, 258, 259, 264, 266, 267, 268, 269, 270, 271, 272, 273, 274, 275, 276, 277, 278, 279, 280, 281 283, 284, 286, 288, 290, 292, 294, 296, 298, 300, 302, 304, 306, 308, 310, 312, 314, 320, 322, 324, 327, 329, 331, and 376 were dissolved (0.02 M) in freshly prepared TFA/H₂O (4:1) solution and stirred at rt mostly for 7 h. The solvent was evaporated by rotatory evaporation at 45° C. to give the desired products 287, 289, 291, 293, 295 297, 299, 301, 303, 305, 307, 311, 313, 315, 321, 323, 325, 328, 330, 332, 377, 384, 385, 386, 387, 388, 389, 390, 391, 392, 393, 394, 395, 396, 397, 398, 399, 400, 401, 402, 403, 404, 405, 406, 407, 408, 684, 686, and 688 as white foams (no, 1 or 2 TFA salts).

### Aldehyde Sidechains

### Preparation of tert-butyl (4-(benzyloxy)benzyl)(3-oxopropyl)carbamate 3-((4-(benzyloxy)benzyl) amino)propan-1-ol (1)

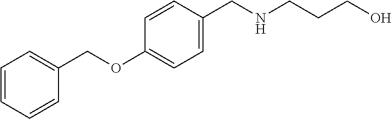 [SMILES: OCCCNCc1ccc(OCc2ccccc2)cc1]

To a mixture of 4-benzyloxybenzaldeyhde (1.00 g, 4.66 mmol) in dry MeOH (4.50 mL) was added dropwise as solution of 3-aminopropanol (0.39 mL, 4.99 mmol) in dry MeOH (4.50 mL). The solution was stirred overnight at ambient temperature. Then, the reaction mixture was cooled down to 0° C. Afterwards, NaBH₄ (0.29 g, 7.46 mmol) was added portion wise. After complete conversion the reaction was concentrated under reduced pressure. The obtained residue was diluted with water and extracted with EtOAc. The combined organic layers were washed with brine, dried over sodium sulfate, and concentrated. The crude product was purified by flash chromatography (CH₂Cl₂/MeOH; 0-10%) to afford the product as colorless oil (0.95 g; 75%).

### tert-butyl (4-(benzyloxy)benzyl)(3-hydroxypropyl)carbamate (2)

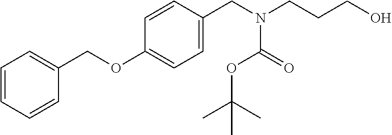 [SMILES: CC(C)(C)OC(=O)N(CCCO)Cc1ccc(OCc2ccccc2)cc1]

To a solution of compound 1 (0.94 g, 3.43 mmol) in CH₂Cl₂ (20.80 mL), Et₃N (0.72 mL, 5.14 mmol) was added. Then, the solution was cooled down in an ice-bath. Afterwards, di-tert-butyl dicarbonate (0.88 mL, 3.77 mmol) was added dropwise to the cooled solution. The reaction mixture was stirred magnetically at rt until complete conversion was monitored by TLC (cyclohexane/EtOAc; 50%). After 4 h, the reaction was diluted with saturated bicarbonate solution and extracted with CH₂Cl₂. The combined organic layers were washed with brine, dried over sodium sulfate, and concentrated under reduced pressure. The obtained crude product was purified by flash chromatography (cyclohexane/EtOAc; 10-80%) which afforded the desired product (1.21 g, 95%).

### tert-butyl (4-(benzyloxy)benzyl)(3-oxopropyl)carbamate (3)

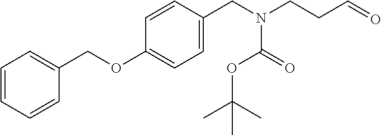 [SMILES: CC(C)(C)OC(=O)N(CCC=O)Cc1ccc(OCc2ccccc2)cc1]

To a solution of oxalyl chloride (0.41 mL, 4.84 mmol) in dry DCM (10.70 mL), DMSO (0.46 mL, 6.45 mmol) was added dropwise at −78° C. After stirring for 20 min at −78° C., a solution of compound 3 in dry DCM (10.70 mL) was added slowly. The solution was stirred at −78° C. for 30 min. Then, triethylamine (2.27 mL, 16.12 mmol) was added slowly and stirred for further 10 min. Afterwards, the reaction mixture was allowed to warm up to rt. The solution was poured onto brine and stirred for 10 min. The organic layer was separated, and the aqueous layer was extracted two times with DCM. The combined organic layers were dried over sodium sulfate and concentrated under vacuum. The obtained residue was purified over silica (cyclohexane, EtOAc, 10-100%) to afford the product (1.05 g, 88%).

### Preparation of tert-butyl (naphthalen-2-ylmethyl)(3-oxopropyl)carbamate 3-((naphthalen-2-ylmethyl) amino)propan-1-ol (4)

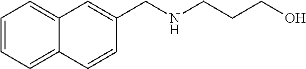 [SMILES: OCCCNCc1ccc2ccccc2c1]

To a mixture of 2-naphtaldehyde (1.00 g, 6.34 mmol) in dry MeOH (6.30 mL) was added dropwise as solution of 3-aminopropanol (0.49 mL, 6.34 mmol) in dry MeOH (6.30 mL). The solution was stirred overnight at ambient temperature. Then, the reaction mixture was cooled down to 0° C. Afterwards, NaBH₄ (0.36 g, 9.51 mmol)) was added portion wise. After complete conversion the reaction was concentrated under reduced pressure. The obtained residue was diluted with water and extracted with EtOAc. The combined organic layers were washed with brine, dried over sodium sulfate, and concentrated. The crude product was purified by flash chromatography (CH₂Cl₂/MeOH; 0-10%) to afford the product 4 as colorless oil (1.02 g; 74%).

### tert-butyl (3-hydroxypropyl)(naphthalen-2-ylmethyl)carbamate (5)

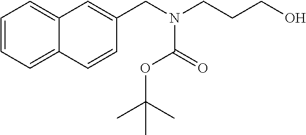 [SMILES: CC(C)(C)OC(=O)N(CCCO)Cc1ccc2ccccc2c1]

To a solution of compound 5 (1.00 g, 4.60 mmol) in CH₂Cl₂ (27.90 mL), Et₃N (0.97 mL, 6.90 mmol) was added. Then, the solution was cooled down in an ice-bath. Afterwards, di-tert-butyl dicarbonate (1.18 mL, 5.06 mmol) was added dropwise to the cooled solution. The reaction mixture was stirred magnetically at rt until complete conversion was monitored by TLC (cyclohexane/EtOAc; 50%). After 4 h, the reaction was diluted with saturated bicarbonate solution and extracted with CH₂Cl₂. The combined organic layers were washed with brine, dried over sodium sulfate, and concentrated under reduced pressure. The obtained crude product was purified by flash chromatography (cyclohexane/EtOAc; 10-80%) which afforded the desired product 6 (1.08 g, 75%).

### tert-butyl (naphthalen-2-ylmethyl)(3-oxopropyl)carbamate (7)

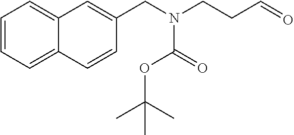 [SMILES: CC(C)(C)OC(=O)N(CCC=O)Cc1ccc2ccccc2c1]

To a solution of oxalyl chloride (0.44 mL, 5.09 mmol) in dry DCM (11.30 mL), DMSO (0.49 mL, 6.79 mmol) was added dropwise at −78° C. After stirring for 20 min at −78° C., a solution of compound 7 in dry DCM (11.30 mL) was added slowly. The solution was stirred at −78° C. for 30 min. Then, triethylamine (2.40 mL, 16.98 mmol) was added slowly and stirred for further 10 min. Afterwards, the reaction mixture was allowed to warm up to rt. The solution was poured onto brine and stirred for 10 min. The organic layer was separated, and the aqueous layer was extracted two times with DCM. The combined organic layers were dried over sodium sulfate and concentrated under vacuum. The obtained residue was purified over silica (cyclohexane, EtOAc, 10-100%) to afford the product (0.66 g, 62%).

### Preparation of tert-butyl (3-oxopropyl)(3-(phenylthio)phenethyl)carbamate 2-(3-(phenylthio)phenyl)-1,3-dioxolane (8)

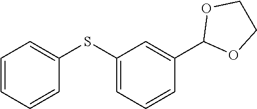 [SMILES: c1ccc(Sc2cccc(C3OCCO3)c2)cc1]

A solution of 2-(3-bromophenyl)-1,3-dioxolane (3 g, 13 mmol) in tetrahydrofuran (50 mL) cooled at −78° C. was treated with tert-butyllithium (26 mmol in pentane), followed by phenydisulphide (3.14 g, 25 mmol) in THF (40 mL). Once the additions were complete the reaction was stirred at −78° C. for one hour and then allowed to warm to room temperature. After one hour at room temperature the reaction was quenched by the addition of water (150 ml). The mixture was concentrated in vacuo and the residue extracted into ether (25 ml×5). The combined extracts were dried (MgSO₄) and evaporated in vacuo. The mixture was purified by flash chromatography (eluding with 10% ethyl acetate/petrol) to afford 8 as a color oil (2.5 g, yield: 77%).

### 3-(phenylthio) benzaldehyde (9)

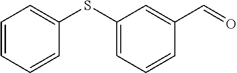 [SMILES: O=Cc1cccc(Sc2ccccc2)c1]

Compound 8 (2.5 g, 9.7 mmol) was dissolved in a mixture solution of ethanol (15 mL), water (15 mL), tetrahydrofuran (15 mL) and sulfuric acid (2 mL), and the solution was stirred for 2.5 hours under reflux. The reaction solution was cooled to 0° C., an aqueous solution of saturated sodium bicarbonate was added thereto. The solution was extracted with ethyl acetate (25 ml×3). The organic layer was washed with brine and dried over anhydrous magnesium sulfate. The solvent was evaporated in vacuo. The residue was purified by silica gel column chromatography (hexane: ethyl acetate=20:1), and the title compound 9 (2 g, 9.3 mmol, 96%) was obtained as a colorless oil. MS Calc.: 214.0; MS Found: 215.0 [M+H]⁺.

### 2-(3-(phenylthio)phenyl)acetaldehyde (10)

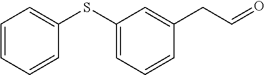 [SMILES: O=CCc1cccc(Sc2ccccc2)c1]

(Methoxymethyl) triphenylphosphonium chloride (12.8 g, 37.4 mmol, 4.0 eq) was suspended in dry THF (0.2 M) and cooled to 0° C. under an argon atmosphere. A solution of potassium tert-butoxide in THF (4.2 g, 37.4 mmol, 4.0 eq) was added slowly to the suspension and allowed to stir for 45 minutes at 0° C. The desired aryl aldehyde 9 (2 g, 9.3 mmol) was added dropwise and the solution was allowed to stir at room temperature for one hour. The reaction was quenched with saturated NH₄Cl (100 mL) and extracted with EtOAc (100 ml×3). The organic phase was dried over MgSO₄, filtered, and concentrated to yield an oil. The oil was then dissolved in a 5:2 THF: 5 N HCl solution (0.2 M) and refluxed for one hour. The solution was cooled to room temperature, quenched with saturated NaHCO₃ and extracted with EtOAc (100 ml×3). The organic layer was dried with MgSO₄, filtered, and concentrated to yield an oil which was purified via silica gel column chromatography eluting with 30:1 PE: EtOAc. and the title compound 10 (0.6 g, yield: 28%) was obtained as a colorless oil. MS Calc.: 228.1; MS Found: 229.1 [M+H]⁺.

### 3-((3-(phenylthio)phenethyl) amino)propan-1-ol (11)

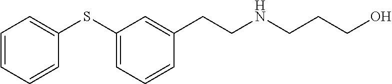 [SMILES: OCCCNCCc1cccc(Sc2ccccc2)c1]

To a solution of compound 10 (600 mg, 2.63 mmol) and in MeOH (10 mL) was added compound 3-aminopropan-1-ol (790 mg, 10.5 mmol)), and the mixture was stirred at room temperature for 0.1 h. Then NaBH₄ (390 mg, 10.5 mmol) was added and the mixture was stirred at room temperature for an additional hour. The reaction was quenched with water and concentrated to dryness. The residue was directly purified by flash (ACN: H₂O=5-95%) to afford compound 11 as an oil (2.5 g, 77%). MS Calc.: 287.1; MS Found: 288.1 [M+H]⁺

### tert-butyl (3-hydroxypropyl)(3-(phenylthio)phenethyl)carbamate (12)

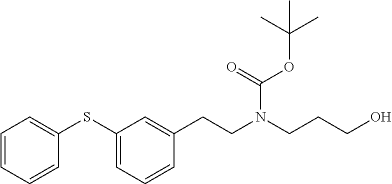 [SMILES: CC(C)(C)OC(=O)N(CCCO)CCc1cccc(Sc2ccccc2)c1]

To a solution of compound 11 (650 mg, 2.26 mmol) in DCM (20 mL) was added TEA (686 mg, 6.8 mmol)) and Boc₂O (739 mg, 3.4 mmol). The mixture was stirred room temperature for 2 hrs. Solvent was removed and the residue was purified by flash chromatography (25% EA in PE) to give compound 12 (650 mg, yield: 74.3%) as a yellow oil. MS Calc.: 387.2; MS Found: 288.2 [M−100+H]⁺.

### tert-butyl (3-oxopropyl)(3-(phenylthio)phenethyl)carbamate (13)

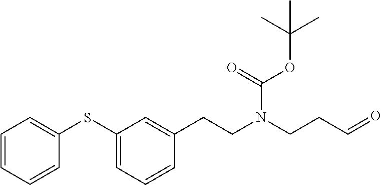 [SMILES: CC(C)(C)OC(=O)N(CCC=O)CCc1cccc(Sc2ccccc2)c1]

To a mixture of compound 12 (650 mg, 1.68 mmol) in DCM (20 mL) was added Dess-Martin Periodinane (1.39 g, 3.36 mmol) at room temperature. After addition the mixture was then stirred for 6 hrs. A mixture of sat. NaHCO₃ (50 mL) and sat. Na2S203 (50 mL) was added into the reaction mixture, which was stirred for 5 min and stood. The organic phase was separated, washed with brine (50 mL), dried and concentrated to provide a crude compound 13 (600 mg, yield: 92.3%), which could be used in the next step. MS Calc.: 385.2; MS Found: 386.2 [M+H]⁺.

### Preparation of tert-butyl (3-oxopropyl)(4-phenethylphenethyl)carbamate (E)-2-(4-styrylphenyl)ethan-1-ol (14)

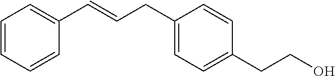 [SMILES: OCCc1ccc(C/C=C/c2ccccc2)cc1]

A suspension of (E)-4,4,5,5-tetramethyl-2-styryl-1,3,2-dioxaborolane (1.0 g, 4.3 mmol), 2-(4-bromophenyl)ethan-1-ol (960 mg, 4.8 mmol), K₂CO₃ (1.2 g, 8.6 mmol), Pd (dppf) Cl₂ (157 mg, 0.22 mmol) in dioxane (20 mL) and H₂O (2 ml) was stirred at 90° C. for 16 hrs under nitrogen atmosphere. The reaction mixture was filtered and the organic phase was concentrated. The crude was purified by flash (A: H₂O; B: MeCN) to give 14 (900 mg, yield: 92%) as a white solid. MS Calc.: 224.1; MS Found: 225.0 [M+H⁺], 207.0 [M−OH].

### 2-(4-phenethylphenyl)ethan-1-ol (15)

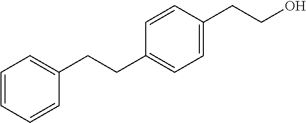 [SMILES: OCCc1ccc(CCc2ccccc2)cc1]

To a solution of 14 (900 mg, 4 mmol) in MeOH (20 mL) was added wet 10% Pd/C(50 mg), and the suspension was stirred at room temperature for 16 hrs. The suspension was filtered and the filtrate was concentrated to give 15 (800 mg, yield: 89%) as a white solid. MS Calc.: 226.1; MS Found: 209.0 [M-OH].

### 2-(4-phenethylphenyl)acetaldehyde (16)

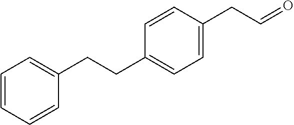 [SMILES: O=CCc1ccc(CCc2ccccc2)cc1]

To a solution of 15 (400 mg, 1.8 mmol) in DCM (10 mL) was added Dess-Martin periodinane (1.1 g, 2.6 mmol) and the solution was stirred at room temperature for 4 hrs. The reaction solution was diluted with DCM (20 mL) and washed with H₂O (20 mL), aq. NαSO₃ (20 ml), aq. NaHCO₃ (20 ml). The organic phase was dried over Na₂SO₄ and concentrated to give 16 (400 mg, crude) as a brown oil.

### 3-((4-phenethylphenethyl)amino)propan-1-ol (17)

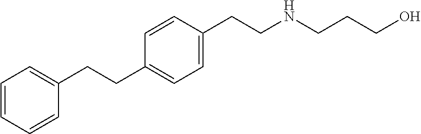 [SMILES: OCCCNCCc1ccc(CCc2ccccc2)cc1]

A solution of 16 (400 mg, 1.8 mmol), 3-aminopropanol (402 mg, 5.4 mmol) in MeOH (5 mL) was stirred at room temperature for 20 min. NaBH₃CN (234 mg, 3.6 mmol) and AcOH (2 drops) was added, the solution was stirred at room temperature for 4 hrs. The resulting mixture was diluted with EA (100 mL), washed with H₂O (100 mL×3) dried over Na₂SO₄. The solution was concentrated to give 17 (400 mg, crude) as a brown oil. MS Calc.: 283.2; MS Found: 284.1 [M+H⁺].

### tert-butyl (3-hydroxypropyl)(4-phenethylphenethyl)carbamate (18)

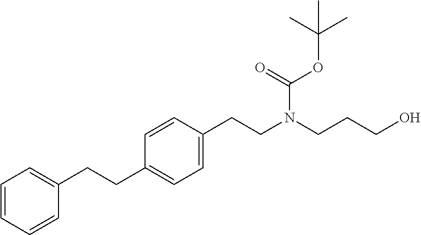 [SMILES: CC(C)(C)OC(=O)N(CCCO)CCc1ccc(CCc2ccccc2)cc1]

To a solution of 17 (400 mg crude), TEA (545 mg, 5.4 mmol) in DCM (10 mL) was added Boc₂O (785 mg, 3.6 mmol). The solution was stirred at room temperature for 16 hrs. The resulting mixture was concentrated and the crude was purified by flash (A: H₂O; B: MeCN) to give 18 (150 mg, three-step yield: 22%) as a clear oil. MS Calc.: 383.2; MS Found: 284.1 [M+H⁺-Boc].

### tert-butyl (3-oxopropyl)(4-phenethylphenethyl)carbamate (19)

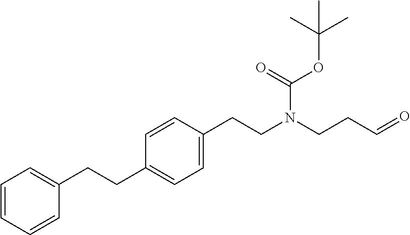 [SMILES: CC(C)(C)OC(=O)N(CCC=O)CCc1ccc(CCc2ccccc2)cc1]

To a solution of 18 (150 mg, 0.39 mmol) in DCM (10 mL), was added Dess-Martin periodinane (249 mg, 0.59 mmol) and the solution was stirred at room temperature for 4 hrs. The reaction mixture was diluted with DCM (20 mL) and washed with H₂O (20 mL), aq. NαSO₃ (20 ml), aq. NaHCO₃ (20 ml). The organic phase was dried over Na₂SO₄ and concentrated to give 19 (100 mg, crude) as a brown oil.

### Preparation of tert-butyl (2-(naphthalen-2-yl)ethyl)(3-oxopropyl)carbamate N-(3-hydroxypropyl)-2-(naphthalen-2-yl)acetamide (20)

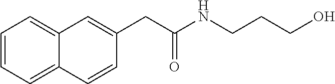 [SMILES: O=C(Cc1ccc2ccccc2c1)NCCCO]

A suspension of 2-(naphthalen-2-yl)acetic acid (3 g, 16.1 mmol), 3-aminopropanol (1.8 g, 24.2 mmol), DIPEA (6.2 g, 48.3 mmol), HATU (9.18 g, 24.2 mmol) in DMF (30 mL) was stirred at room temperature for 2 hrs. The resulting solution was concentrated to dryness and the residue was diluted with EtOAc (100 mL) and washed with water (50 mL×3). Then the organic phase was dried over Na2SO₄ and concentrated. The residue was purified by reverse phase flash (MeCN/H₂O) to afford 20 (3.5 g, yield: 90%) as a yellow oil. MS Calc.: 243.1; MS Found: 244.1 [M+H]⁺.

### 3-((2-(naphthalen-2-yl)ethyl) amino)propan-1-ol (21)

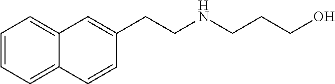 [SMILES: OCCCNCCc1ccc2ccccc2c1]

To a mixture of compound 20 (3.5 g, 14.4 mmol) in THF (50 mL) was added BH3. DMS (2.9 mL, 28.8 mmol) dropwise. Then the mixture was stirred at 65° C. for 4 hrs. The mixture was quenched by adding MeOH and then concentrated. The residue was purified by reverse phase flash (MeCN/H₂O) to afford 21 (2 g, yield: 60.4%) as a yellow oil. MS Calc.: 229.1; MS Found: 230.1 [M+H]⁺.

### tert-butyl (3-hydroxypropyl)(2-(naphthalen-2-yl)ethyl)carbamate (22)

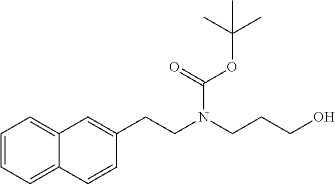 [SMILES: CC(C)(C)OC(=O)N(CCCO)CCc1ccc2ccccc2c1]

To a solution of compound 22 (2 g, 8.7 mmol) in DCM (30 mL) was added TEA (2.64 g, 26.1 mmol)) and Boc₂O (2.84 g, 13 mmol). The mixture was stirred at room temperature for 2 hrs. Solvent was removed and the residue was purified by flash chromatography (25% EA in PE) to give compound 23 (2.5 g, yield: 87.3%) as a yellow oil. MS Calc.: 329.1; MS Found: 230.1 [M−100+H]⁺

### tert-butyl (2-(naphthalen-2-yl) ethyl)(3-oxopropyl)carbamate (24)

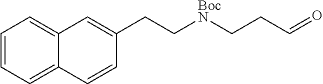 [SMILES: CC(C)(C)OC(=O)N(CCC=O)CCc1ccc2ccccc2c1]

To a mixture of compound 23 (2.5 g, 7.6 mmol) and in DCM (50 mL) was added Dess-Martin periodinane (6.3 g, 15.2 mmol) at room temperature. After addition the mixture was then stirred for 6 hrs at room temperature. A mixture of sat. NaHCO₃ (50 mL) and sat. Na2S203 (50 mL) was added into the reaction mixture, which was further stirred for 5 min and stood. The organic phase was separated, washed with brine (50 mL) and dried over Na₂SO₄. The solution was concentrated to provide a crude compound 24 (2.5 g, quantitative yield), which could be used in the next step. MS Calc.: 327.2; MS Found: 328.1 [M+H]⁺.

### Preparation of tert-butyl (3-oxopropyl)(4-phenoxybenzyl)carbamate 3-((4-phenoxybenzyl) amino)propan-1-ol (25)

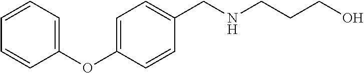 [SMILES: OCCCNCc1ccc(Oc2ccccc2)cc1]

4-Phenoxybenzaldehyde (0.51 g, 2.55 mmol, 1.03 equiv.) was dissolved in 37 ml dry MeOH under N₂ atmosphere. Then, 3-aminopropan-1-ol (0.19 ml, 0.187 g, 2.48 mmol, 1.0 equiv) was added dropwise at room temperature and the resulted mixture was stirred overnight. After 19 h stirring, the reaction mixture was cooled down to 0° C. and NaBH₄ (0.144 g, 3.81 mmol, 1.53 equiv.) was added portionwise. After the reaction was stirred at RT for 5 h, the solvent was removed under reduced pressure. Water was added and was extracted with EtOAc (3x), the combined organic phases were washed with brine (1x) and dried over Na₂SO₄. After filtration, the solvent was removed under vacuum and the residue was purified by flash chromatography eluting CH₂Cl₂/MeOH (0.4% to 8%) to afford the product as a yellowish oil. Yield: 0.43 g, 67%). C₁₆H₁₉NO₂ (257.33 g/mol). APCI: calc. For C₁₆H₁₉NO₂ [M+H]⁺: 257.14 found: 257.9. ¹H NMR (400 MHz, DMSO-d₆) δ 7.42-7.26 (m, 4H, o-H, m-H′), 7.14-7.05 (m, 1H, p-H′), 7.04-6.89 (m, 4H, m-H, o-H′), 3.64 (s, 2H, Ar—CH₂—N), 3.45 (t, J=6.3 Hz, 2H, H₁′), 3.39 (bs, 1H, NH), 2.56-2.44 (m, 2H, H₃′), 1.57 (p, J=12 Hz, 2H, H₂′).

### tert-butyl (3-hydroxypropyl)(4-phenoxybenzyl)carbamate (26)

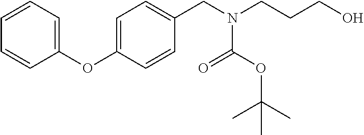 [SMILES: CC(C)(C)OC(=O)N(CCCO)Cc1ccc(Oc2ccccc2)cc1]

Compound 25 (0.43 g, 1.66 mmol, 1.0 equiv.) was dissolved in 10 ml dry DCM. Then triethylamine (0.35 ml, 0.26 g, 2.52 mmol, 1.52 equiv.) was added and the reaction was cooled down to 0° C. After that, Boc₂O (0.43 ml, 0.41 g, 1.87 mmol, 1.13 equiv.) was dropwise added to the mixture. The reaction was stirred at room temperature for 3 h (monitored by TLC; DCM/MeOH 9:1). After that, the reaction mixture was filtered over cotton and silica, washed with EtOAC and the solvents were evaporated. The residue was purified by flash chromatography eluting Cyclohexane/EtOAc (5%-50%) to afford the product as a transparent oil (yield: 0.529 g, 89%). ¹H NMR (400 MHZ, DMSO-d₆) δ 7.41-7.34 (m, 2H, m-H′), 7.24-7.21 (m, 2H, o-H), 7.14-7.05 (m, 1H, p-H′), 7.01-6.97 (m, 4H, o-H′, m-H), 4.41 (t, J=5.1 Hz, 1H, OH), 4.34 (bs, 2H, Ar—CH₂—N), 3.36 (td, J=6.3 Hz, 4.9 Hz, 2H, H₁′), 3.15 (bs, 2H, H₃′), 1.66-1.53 (m, 2H, H₂′), 1.47-1.26 (m, 9H, t-Bu, Boc)

### tert-butyl (3-oxopropyl)(4-phenoxybenzyl)carbamate (27)

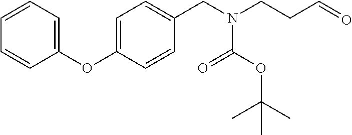 [SMILES: CC(C)(C)OC(=O)N(CCC=O)Cc1ccc(Oc2ccccc2)cc1]

Under N₂ atmosphere, solution of oxalyl-chloride (89 μL, 0.13 g, 1.05 mmol, 1.50 equiv.) in 1.4 ml dry DCM was cooled down to −78° C. Then solution of DMSO (0.1 ml, 0.11 g, 1.41 mmol, 2.01 equiv.) in 0.6 ml dry DCM was added. It was stirred for 20 min at −78° C. Then, solution of 26 (0.25 g, 0.70 mmol, 1.0 equiv.) in 1 ml dry DCM was added dropwise at −78° C. After the reaction mixture was stirred at −78° C. for 1 h, triethylamine (0.49 ml, 0.36 g, 3.53 mmol, 5.05 eqiv.) was slowly added and was stirred at −78° C. for 20 min. Then the reaction was allowed to warm up to room temperature and 5% citric acid (aq.) solution was added. The mixture was extracted with DCM (3x), the combined organic phases were washed with saturated NaHCO₃(aq.) solution (1x) and dried over Na₂SO₄. After filtration, the solvent was removed under vacuum and the residue was purified by flash chromatography eluting Cyclohexane/EtOAc (2% to 50%) to afford the product as an oil (yield: 0.106 g, 43%). ¹H NMR (400 MHZ, DMSO-d₆) δ 9.64 (t, J=1.8 Hz, 1H, CHO), 7.43-7.37 (m, 2H, m-H′), 7.31-7.20 (m, 2H, o-H), 7.14-7.05 (m, 1H, p-H′), 7.01-6.97 (m, 4H, o-H′, m-H), 4.36 (s, 2H, Ar—CH₂—N), 3.39 (bs, 2H, H₃′), 2.62 (td, J=6.7 Hz, 1.9 Hz, 2H, H₂′), 1.50-1.32 (m, 10H, t-Bu, Boc).

### Preparation of 2-(2,4-difluorophenyl)acetaldehyde 2-(2,4-difluorophenyl)ethan-1-ol (28)

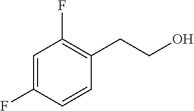 [SMILES: OCCc1ccc(F)cc1F]

1 M solution of LiAlH₄ (2.3 ml, 2.30 mmol, 2.0 equiv.) was charged into a previously N₂ flushed round bottom flask and it was cooled to 0° C. Then solution of 2-(2,4-difluorophenyl)acetic acid (0.203 g, 1.18 mmol, 1.0 equiv.) in 4.4 ml dry THF was added dropwise over 4 min to the LiAlH₄ solution at 0° C. The reaction was stirred at ambient temperature for 6.5 h. Then the reaction mixture was cooled down to 0° C. and 0.1 ml H₂O was added carefully during stirring and it was stirred for 50 min. After that, 0.1 ml 15 w/w % NaOH (aq.) solution and 0.2 ml H₂O was added and stirred overnight. On the next morning (after 11 h stirring), the reaction mixture was filtered through celite and washed with THF. Then the filtrate was concentrated under vacuum. The residue was purified by flash chromatography eluting cyclohexane/ethylacetate (1%-25%) to afford the product (0.095 g, 52%) as a yellow oil. R^{f}=0.16 (cyclohexane/EtOAc 8:2). ¹H NMR (400 MHz, DMSO-d₆) δ 7.34 (q, J=6.9 Hz, 1H,), 7.14 (m, 1H,), 6.99 (tdd, J=8.6, 2.6, 0.9 Hz, 1H,), 4.71 (t, J=5.3 Hz, 1H, OH), 3.55 (td, J=6.9, 5.5 Hz, 2H, CH₂—OH), 2.71 (t, J=6.9 Hz, 2H, Ph-CH₂)

### 2-(2,4-difluorophenyl)acetaldehyde (29)

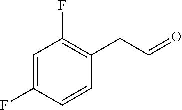 [SMILES: O=CCc1ccc(F)cc1F]

To a suspension of Dess-Martin periodinane (0.277 mg, 0.65 mmol, 1.2 equiv.) in anhydrous CH₂Cl₂ (5 ml) was added dropwise a solution of 28 (0.086 mg, 0.54 mmol, 1 equiv.) in anhydrous CH₂Cl₂ (1.7 ml) at 0° C. The resulted reaction mixture was stirred at room temperature for 18.7 h. Then the reaction was quenched with 1.7 M Na₂S₂O₃ (aq.) solution (5.4 ml) and stirred for 15 min. The two layers were separated and the aqueous layer was extracted with CH₂Cl₂ (2x), then the combined organic layers were washed with 5% NaHCO₃(aq.) solution (1x) and with brine (1x), and dried over Na₂SO₄. After filtration, the solvent was removed under vacuum and the residue was purified by flash chromatography eluting Cyclohexane/EtOAc (2%-12%) to afford the product (0.020 g, 23%) as a clear liquid.

### Preparation of 2-(4-(trifluoromethyl)phenyl)acetaldehyde 2-(4-(trifluoromethyl)phenyl)acetaldehyde (30)

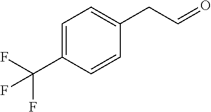 [SMILES: O=CCc1ccc(C(F)(F)F)cc1]

Dess-Martin periodinane (0.54 g, 1.25 mmol, 1.2 equiv.) was suspended in 13 ml dry CH₂CL2 and it was cooled down to 0° C. Then 0.16 ml 2-(4-(trifluoromethyl)phenyl)ethan-1-ol(0.20 g, 1.04 mmol, 1 equiv.) was added dropwise to the suspension. The mixture was stirred at room temperature for 15.5 h, then 1 M Na₂S₂O₃ (aq.) solution was added and the phases were separated. The water phase was extracted with CH₂CL2 (2x), the combined organic phases were washed with 5% NaHCO₃(aq.) solution (2×) and with brine (1x), dried over Na₂SO₄, filtered and the solvent was evaporated. The residue was purified by flash chromatography eluting Cyclohexane/EtOAc (2%-15%) to afford the product as a yellow liquid (0.095 mg, 48%). R^{f}=0.4 (Cyclohexane/EtOAc 8:2). ¹H NMR (400 MHZ, DMSO-d₆) δ 9.73 (t, J=1.5 Hz, 1H, —CHO), 7.72 (d, J=8.1 Hz, 2H, m-H), 7.48 (d, J=8.1 Hz, 2H, o-H,), 3.95 (s, 2H, —CH₂)

### Preparation of tert-butyl (3-oxopropyl)(3-phenoxyphenethyl)carbamate 3-((3-phenoxyphenethyl) amino)propan-1-ol (31)

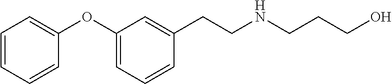 [SMILES: OCCCNCCc1cccc(Oc2ccccc2)c1]

To a solution of compound 2-(3-phenoxyphenyl)acetaldehyde (2 g, 9.4 mmol) in MeOH (20 mL) was added 3-aminopropan-1-ol (1.41 g, 18.8 mmol)), and the mixture was stirred at room temperature for 1 hr. Then NaBH₃CN (2.87 g, 47 mmol) was added and the mixture was stirred overnight. Solvent was removed and the residue was purified by flash chromatography (30% EA in PE) to give compound 31 (1 g, yield: 39.2%) as a yellow oil. MS Calc.: 271.2; MS Found: 272.2 [M+H]⁺

### tert-butyl (3-hydroxypropyl)(3-phenoxyphenethyl)carbamate (32)

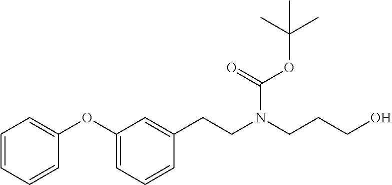 [SMILES: CC(C)(C)OC(=O)N(CCCO)CCc1cccc(Oc2ccccc2)c1]

To a solution of compound 31 (1 g, 3.7 mmol) in DCM (20 mL) was added TEA (1.1 g, 11.1 mmol) and (Boc) 2O (1.2 g, 5.5 mmol). The mixture was stirred at room temperature for 2 hrs. Solvent was removed and the residue was purified by flash chromatography (25% EA in PE) to give compound 32 (1.3 g, yield: 94.7%) as a yellow oil. MS Calc.: 371.2; MS Found: 272.1 [M-100+H]⁺.

### tert-butyl (3-oxopropyl)(3-phenoxyphenethyl)carbamate (33)

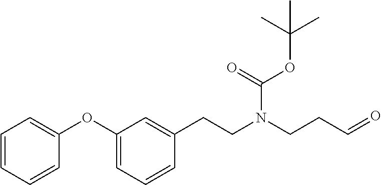 [SMILES: CC(C)(C)OC(=O)N(CCC=O)CCc1cccc(Oc2ccccc2)c1]

To a mixture of compound 32 (1.3 g, 3.5 mmol) in DCM (20 mL) was added Dess-Martin periodinane (2.9 g, 7 mmol), and the mixture was at room temperature stirred for 1 hr. A mixture of sat. NaHCO₃ (20 mL) and sat. Na₂S203 (20 mL) was added into the reaction mixture, which was stirred for 5 min and separated. The organic phase was washed with brine (10 mL), dried over Na₂SO₄ and concentrated to provide a crude compound 33 (1 g, yield: 77%), which was used for the next step without further purification. MS Calc.: 369.2; MS Found: 370.2 [M+H]⁺.

### Preparation of tert-butyl (4-(4-chlorophenoxy)-3-fluorophenethyl)(3-oxopropyl)carbamate 2-fluoro-4-(2-hydroxyethyl)phenol (34)

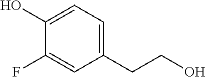 [SMILES: OCCc1ccc(O)c(F)c1]

To a heat dried three-necked round bottom flask equipped with a thermometer and a dropping funnel, NaBH₄ (2.65 g, 70.00 mmol) was added in dry THF (60.00 mL). To this mixture, Me2SO₄ (6.64 mL, 70.00 mmol) was slowly added at 0° C. and stirred for 1 h at 0° C. Then, the mixture was allowed to warm up to rt and stirred for 3 h. Afterwards, a solution of 2-(3-fluoro-4-hydroxyphenyl)acetic acid (5.95 g, 35.00 mmol) and B(OMe)₃ (7.91 mL, 70.00 mmol) in dry THF (10 mL) were added slowly added at rt while cooling the flask with an ice-bath. The mixture was stirred overnight at rt. TLC(100% EtOAc) monitored the formation of a new spot. The mixture was cooled down in an ice-bath and stirred vigorously while water was carefully added. Then, the organic solvent was evaporated under reduced pressure. The aqueous phase was extracted three times with EtOAc. The combined organic layers were washed three times with saturated sodium bicarbonate solution and three times with brine. The separated organic layer was dried over sodium sulfate and concentrated under reduced pressure. ¹H NMR (400 MHZ, DMSO-d₆) δ9.53 (s, Ar—OH 1H), 6.98 (dd, J=12.7, 1.8 Hz, 1H, Ar—H), 6.89-6.77 (m, 2H—Ar—H), 4.61 (t, J=5.1 Hz, 1H, OH), 3.55 (td, J=7.0, 5.1 Hz, 2H, CH₂), 2.61 (t, J=7.0 Hz, 2H, CH₂). APCI-MS (+): m/z 157.0 [M+H]⁺.

### 2-(4-(4-chlorophenoxy)-3-fluorophenyl)ethan-1-ol (35)

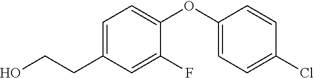 [SMILES: OCCc1ccc(Oc2ccc(Cl)cc2)c(F)c1]

In a heat-dried three-necked round bottom flask, 4-Chlorophenylboronic acid (3.05 g, 19.50 mmol) and 34 (1.0 g, 6.50 mmol) were added in dry C₂H₂Cl₂ (40.60 mL). Afterwards, dry pyridine (1.51 mL, 19.50 mmol), anhydrous copper acetate (1.77 g, 9.75 mmol), and 4 Å molecular sieves (1.12 g) were added to the mixture. The reaction mixture was stirred for 48 h at rt. The mixture was filtered over celite and the cake was rinsed several times with CH₂Cl₂. The filtrate was concentrated under vacuum to complete dryness. The obtained residue was purified over silica (cyclohexane/EtOAc; 10-80%) to afford the pure product (0.93 g, 54%). ¹H NMR (400 MHZ, DMSO-d₆) δ7.46-7.37 (m, 2H, H 2,6), 7.28 (dd, J=12.1, 1.9 Hz, 1H, 6′H), 7.15 (t, J=8.3 Hz, 1H, 2′H), 7.10 (dd, J=8.5, 1.9 Hz, 1H, 5′H), 6.99-6.94 (m, 2H, 2,6 H), 4.69 (t, J=5.2 Hz, 1H, OH), 3.64 (td, J=6.8, 5.2 Hz, 2H, CH₂), 2.75 (t, J=6.8 Hz, 2H, CH₂). APCI-MS (+): m/z 265.1/268.1 [M+H]⁺.

### 4-(4-chlorophenoxy)-3-fluorophenethyl 4-methylbenzenesulfonate (36)

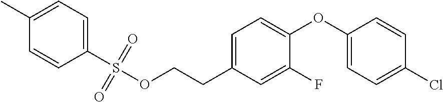 [SMILES: Cc1ccc(S(=O)(=O)OCCc2ccc(Oc3ccc(Cl)cc3)c(F)c2)cc1]

The compound 35 (1.28 g, 4.80 mmol) was dissolved in dry CH₂Cl₂ (24.00 mL). Then, TsCl (0.92 g, 4.80 mmol) was added. Afterwards, dry pyridine (1.16 mL, 14.34 mmol) and DMAP (0.06 g, 0.48 mmol) were added 0° C. The solution was stirred at rt overnight. TLC(cyclohexane/EtOAc; 20%) monitored no full conversion. In addition, dry pyridine (0.58 mL, 7.20 mmol) was added and stirred at rt till complete conversion. After 6 h, the mixture was diluted with saturated bicarbonate solution and extracted three times with CH₂Cl₂. The combined organic layers were washed with brine, dried over sodium sulfate, and concentrated under reduced pressure. The crude was purified by flash chromatography (cyclohexane/EtOAc; 0-80%) to afford the product as a colorless oil (1.33 g, 75%). ¹H NMR (400 MHZ, DMSO-d₆) δ7.73-7.68 (m, 2H, o-tosyl), 7.48-7.38 (m, 4H, m-tosyl, H_{3/5}), 7.19 (dd, J=12.0, 2.0 Hz, 1H, H₅’), 7.10 (t, J=8.4 Hz, 1H, H₂′), 7.02 (dd,)=8.3, 1.3 Hz, 1H, H₆′), 7.02-6.93 (m, 2H, H_{2/6}), 4.28 (t, J=6.3 Hz, 2H, CH₂), 2.92 (t, J=6.3 Hz, 2H, CH₂), 2.41 (s, 3H, CH₃). APCI-MS (+): m/z 420.0/422.1 [M+H]⁺.

### 3-((tert-butyldiphenylsilyl)oxy)propan-1-amine (37)

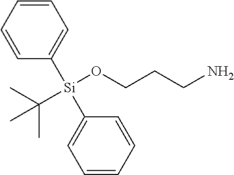 [SMILES: CC(C)(C)[Si](OCCCN)(c1ccccc1)c1ccccc1]

3-amino-1-propanol (2.07 mL, 27.29 mmol) was dissolved in dry CH₂Cl₂ (91.00 mL) and cooled down in an ice-bath. Then, TBDPS-Cl (4.73 mL, 18.19 mmol) was added dropwise at 0° C. Afterwards, triethylamine (3.78 mL, 27.29 mmol) was added. The solution was allowed to warm up to rt and the reaction was stirred magnetically at rt overnight. The reaction mixture was diluted with saturated bicarbonate solution and extracted with CH₂Cl₂. The combined organic layers were washed with brine, dried over sodium sulfate, and concentrated over vacuum. The obtained crude was purified over silica (CH₂Cl₂/MeOH; 0-20%) to afford the pure product as a colorless oil (3.42 g, 60%). ¹H NMR (400 MHZ, DMSO-d₆) δ 7.67-7.58 (m, 4H, o-Ar—H), 7.52-7.39 (m, 6H, m,p-Ar—H), 3.71 (t, J=6.3 Hz, 2H, CH₂, H₃), 2.64 (t, J=6.8 Hz, 2H, CH₂, H₁), 1.61 (p, J=6.5 Hz, 2H, CH₂, H₂), 1.00 (s, 9H, CH₃, t-butyl). APCI-MS (+): m/z 314.0 [M+H]⁺.

### 3-((tert-butyldiphenylsilyl)oxy)-N-(4-(4-chlorophenoxy)-3-fluorophenethyl)propan-1-amine (38)

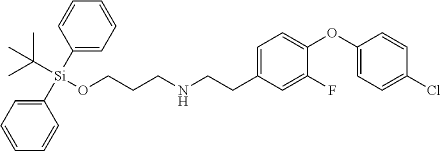 [SMILES: CC(C)(C)[Si](OCCCNCCc1ccc(Oc2ccc(Cl)cc2)c(F)c1)(c1ccccc1)c1ccccc1]

A solution of 36 (1.05 g, 2.51 mmol) in dry DMF (6.25 mL) was added to a solution of 37 (1.18 g, 3.76 mmol) in dry DMF (6.25 mL) at rt. Then, CsCO₃ (1.77 g, 5.01 mmol) was added and the suspension was heated to 80° C. and stirred at this temperature for 5 h. TLC(cyclohexane/EtOAc; 50%) monitored full consumption of 35. Then, the reaction was allowed to cool down to rt and was diluted with saturated bicarbonate solution. The aqueous phase was extracted three times with EtOAc. Afterwards, the combined organic layers were washed with brine, dried over sodium sulfate, and concentrated under reduced pressure. The obtained residue was purified by flash chromatography (cyclohexane/EtOAc; 0-60%) to afford the pure product. ¹H NMR (400 MHZ, DMSO-d₆) δ7.64-7.60 (m, 2H, Ar—H), 7.51-7.35 (m, 8H, Ar—H, H_{3/5}), 7.26 (dd, J=12.0, 1.9 Hz, 1H, H₅′), 7.15-7.08 (m, 1H, H₂′), 7.06 (dd, J=8.4, 1.8 Hz, 1H, H₆′), 7.00-6.91 (m, 2H, H_{2/6}), 3.71 (t, J=6.3 Hz, 2H, CH₂, H₅″), 2.77-2.66 (m, 4H, CH₂, H₁″/H₂″), 2.63 (t, J=6.8 Hz, 2H, CH₂, H₃″), 1.67 (p, J=6.5 Hz, 2H, CH₂, H₄″), 1.00 (s, 9H, CH₃, t-butyl). APCI-MS (+): m/z 561.8/563.8 [M+H]⁺.

### 3-((4-(4-chlorophenoxy)-3-fluorophenethyl) amino)propan-1-ol(39)

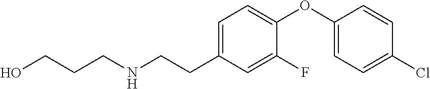 [SMILES: OCCCNCCc1ccc(Oc2ccc(Cl)cc2)c(F)c1]

To a solution of 38 (0.67 g, 1.19 mmol) in dry THF (5.90 mL), TBAF (0.65 mL, 2.38 mmol) was added dropwise. Then, the solution was stirred magnetically at rt for 5 h. TLC(CH₂Cl₂/MeOH; 10%) indicated full conversion. After 5 h, the organic solvent was evaporated under vacuum and the obtained residue was purified over silica (CH₂Cl₂/MeOH; 0-10%) to afford the desired product (0.23 g, 60%). ¹H NMR (400 MHZ, DMSO-d₆) δ7.44-7.39 (m, 2H, H_{2/6}), 7.29 (dd, J=12.1, 1.9 Hz, 1H, H₅′), 7.15 (t, J=8.3 Hz, 1H, H₂′), 7.09 (dd, J=8.2, 1.8 Hz, 1H, H₆′), 6.99-6.95 (m, 2H, H_{3/5}), 3.45 (t, J=6.3 Hz, 2H, CH₂, H₅″), 2.82-2.68 (m, 4H, CH₂, H_{1″/2}″), 2.61 (t, J=6.9 Hz, 2H, CH₂, H₃″), 1.56 (p, J=6.6 Hz, 2H, CH₂, H₄″). APCI-MS (+): m/z 324.0/326.0 [M+H]⁺.

### tert-butyl (4-(4-chlorophenoxy)-3-fluorophenethyl)(3-hydroxypropyl)carbamate (40)

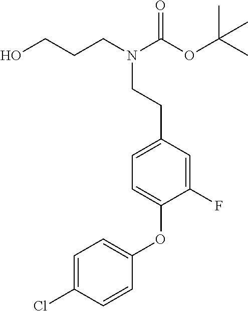 [SMILES: CC(C)(C)OC(=O)N(CCCO)CCc1ccc(Oc2ccc(Cl)cc2)c(F)c1]

Triethylamine (0.14 mL, 1.02 mmol) was added to a solution of 39 (0.22 g, 0.68 mmol) in dry CH₂Cl₂ (3.40 mL). The solution was cooled down to 0° C. in an ice-bath. Afterwards, di-tert-butyl dicarbonate (0.17 mL, 0.75 mmol) was added portion wise. Then, the reaction mixture was allowed to warm up to rt and stirred for 5 h till complete conversion was monitored by TLC (cyclohexane/EtOAc; 50%). The mixture was diluted with saturated sodium bicarbonate solution and the organic phase was separated. The aqueous phase was extracted three times with CH₂Cl₂. The combined organic layers were washed with brine, dried over sodium sulfate, and concentrated over vacuum. The crude was purified by flash chromatography (cyclohexane/EtOAc; 10-80%) to afford a white solid (0.26 g, 89%). ¹H NMR (400 MHZ, DMSO-d₆) δ7.44-7.38 (m, 2H, H2/6), 7.26 (dd, J=11.9, 2.0 Hz, 1H, H5′), 7.17 (t, J=8.5 Hz, 1H, H2′), 7.06 (d, J=8.3 Hz, 2H, H6′), 6.99-6.94 (m, 2H, H3/5), 4.43 (t, J=5.0 Hz, 1H, OH), 3.42-3.35 (m, 4H, CH₂, H2″/5″), 3.21-3.10 (m, 2H, CH₂, H3″), 2.79 (t, J=7.2 Hz, 2H, CH₂, H1″), 1.60 (p, J=6.5 Hz, 2H, CH₂, H4″), 1.35 (s, 9H, CH₃, t-butyl). APCI-MS (+): m/z 324.2/326.2 [M+H]+ without Boc-group.

tert-butyl (4-(4-chlorophenoxy)-3-fluorophenethyl)(3-oxopropyl)carbamate (41)

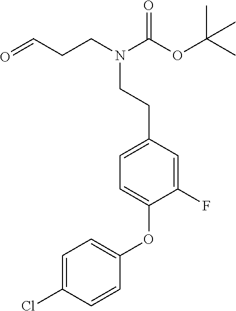 [SMILES: CC(C)(C)OC(=O)N(CCC=O)CCc1ccc(Oc2ccc(Cl)cc2)c(F)c1]

To a solution of 40 (0.06 g, 0.14 mmol) in dry DMSO (0.70 mL), triethylamine (0.04 mL, 0.28 mmol) was added at rt. Then, a solution of sulfur trioxide pyridine complex (0.05 g, 0.28 mmol) in dry DMSO (0.70 mL) was added while vigorously stirring. TLC(cyclohexane/EtOAc; 50%) monitored conversion. After 1 h, the solution was quenched with water at 0° C. and the aqueous phase was extracted with CH₂Cl₂. The combined organic layers were washed with brine, dried over sodium sulfate, and concentrated under reduced pressure. The crude was purified over silica (cyclohexane/EtOAc; 10-60%) to afford the product (0.06 g, 100%) as a yellowish resin. ¹H NMR (400 MHZ, DMSO-d₆) δ9.66 (s, 1H, R-CHO), 7.45-7.37 (m, 2H, H2/6), 7.27 (dd, J=12.0, 1.8 Hz, 1H, H5), 7.17 (t, J=8.5 Hz, 1H, H2), 7.07 (dd, J=8.0, 1.3 Hz, 1H, H6), 3.45-3.36 (m, 4H, CH₂, H2″/3″), 2.78 (t, J=7.2 Hz, 2H, CH₂, H1″), 2.63 (td, J=6.6, 1.7 Hz, 2H, CH₂, H4″), 1.33 (s, 9H, CH₃, t-butyl). APCI-MS (+): m/z 322.1/324.1 [M+H]+ without Boc-group.

### Preparation of tert-butyl (3-oxopropyl)(phenethyl)carbamate 3-(phenethylamino)propan-1-ol (42)

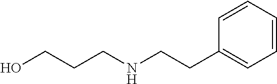 [SMILES: OCCCNCCc1ccccc1]

To a solution of 2-phenylacetaldehyde (0.60 g, 5.00 mmol) in dry MeOH (10 mL) was added 3-aminopropan-1-ol (0.38 g, 5.00 mmol) dropwise at rt. The reaction mixture was stirred overnight at rt. TLC monitored complete conversion. The reaction was then cooled down in an ice-bath. Then, NaBH₄ (0.28 g, 7.50 mmol) was added portion wise to the solution. After the bubbling hat stopped, the solvent was evaporated under vacuum. The obtained residue was diluted with water and extracted with EtOAc. The organic layer was washed with brine, dried over sodium sulfate, and concentrated under reduced pressure. The obtained crude was purified by flash chromatography (EtOAc 100%, then DCM/MeOH; 0-20%) to afford the product 42 (0.82 g, 92%). ¹H NMR (400 MHZ, DMSO-d₆) δ7.31-7.25 (m, 1H), 7.23-7.15 (m, 2H), 3.44 (t, J=6.3 Hz, 1H), 2.70 (dt, J=4.4, 2.4 Hz, 3H), 2.58 (t, J=6.8 Hz, 1H), 1.54 (p, J=6.6 Hz, 1H).

### tert-butyl (3-oxopropyl)(phenethyl)carbamate (43)

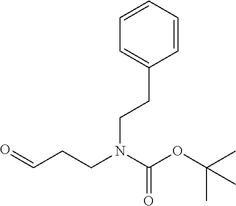 [SMILES: CC(C)(C)OC(=O)N(CCC=O)CCc1ccccc1]

To a solution of 42 (0.59 g, 3.29 mmol) in dry DCM (16.5 mL), triethylamine (0.68 mL, 4.94 mmol). Then, the solution was cooled down in an ice-bath and stirred at this temperature for 10 min. Afterwards, di-tert-butyl dicarbonate (0.83 mL, 3.62 mmol) was added portionwise to the cooled solution. The reaction mixture was allowed to warm up to rt and stirred magnetically at this temperature until TLC(cyclohexane/EtOAc; 50% and DCM/MeOH: 20%) monitored complete conversion. After the reaction was completed, the mixture was filtrated over silica and washed with EtOAc. The filtrate was then concentrated under reduced pressure. Step 2: To a solution of oxalyl chloride (0.42 mL, 4.94 mmol) in dry DCM (14.0 mL), DMSO (0.70 mL, 9.87 mmol) was added dropwise at −78° C. After stirring for 20 min at −78° C., a solution of the boc-protected amine in dry DCM (2.5 mL) was added slowly. The solution was stirred at −78° C. for 30 min. Then, triethylamine (2.05 mL, 14.81 mmol) was added slowly and stirred for further 10 min. Afterwards, the reaction mixture was allowed to warm up to rt. The solution was poured onto brine and stirred for 10 min. The organic layer was separated, and the aqueous layer was extracted two times with DCM. The combined organic layers were dried over sodium sulfate and concentrated under vacuum. The obtained residue was purified over silica (cyclohexane, EtOAc, 0-35%) to afford the product 43 (0.58 g, 64%). ¹H NMR (400 MHZ, DMSO-d₆) δ 9.65 (t, J=1.8 Hz, 1H, CHO), 7.34-7.24 (m, 2H, o′), 7.24-7.14 (m, 3H, m′, p′), 3.44-3.37 (m, 2H, CH₂, H2′), 3.37-3.31 (m, 2H, CH₂, H3′), 2.79-2.71 (m, 2H, CH₂, H4′), 2.61 (td, J=6.7, 1.9 Hz, 2H, CH₂, H1′), 1.45-1.25 (m, 9H CH₃, (Bu).

### Preparation of tert-butyl (3-(methyl(phenyl)amino)phenethyl)(3-oxopropyl)carbamate 2-(3-(methyl(phenyl)amino)phenyl)ethan-1-ol (44)

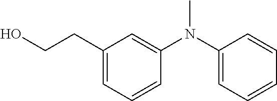 [SMILES: CN(c1ccccc1)c1cccc(CCO)c1]

A heat-dried three-necked round bottom flask was charged with Pd₂ (dba)₃ (0.20 g, 0.22 mmol) and cyclohexyl-JohnPhos (0.09 g, 0.26 mmol) under inert atmosphere. Then, dry degassed THF (55.10 mL) was added. To this stirred suspension were added 2-(3-bromophenyl)ethan-1-ol (1.50 g, 11.02 mmol), methylaniline (1.82 g, 13.22 mmol), and a 1M solution of LIN (TMS) 2 (4.10 g, 24.24 mmol) in THF. The resulted mixture was degassed and heated to 65° C. After 18 h, the mixture was cooled down to rt before diluted with saturated bicarbonate solution. The aqueous phase was extracted with EtOAc. The combined organic layers were washed with brine, dried over sodium sulfate, and concentrated under reduced pressure. The obtained residue was purified over silica (cyclohexane/EtOAc; 0-30%) to afford the desired compound as an orange resin (0.95 g, 38%). ¹H NMR (400 MHZ, DMSO-d₆) δ7.27-7.21 (m, 2H, H3, H4), 7.16 (t, J=7.8 Hz, 1H, H5′), 6.98-6.93 (m, 2H, H2, H6), 6.92-6.87 (m, 1H, H4), 6.87-6.85 (m, 1H, H2′), 6.84-6.78 (m, 2H, H4′, H6′), 4.60 (t, J=5.3 Hz, 1H, OH), 3.56 (td, J=7.1, 5.3 Hz, 2H, CH₂, H1″), 3.23 (s, 3H, CH₃, N-Me), 2.64 (t, J=7.1 Hz, 2H,, CH₂, H2″).

### 3-(methyl(phenyl) amino)phenethyl 4-methylbenzenesulfonate (45)

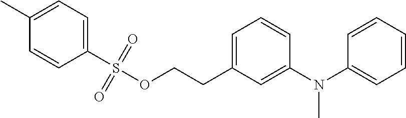 [SMILES: Cc1ccc(S(=O)(=O)OCCc2cccc(N(C)c3ccccc3)c2)cc1]

The alcohol 44 (0.95 g, 4.14 mmol) and TsCl (0.80 g, 4.14 mmol) were dissolved in CH₂Cl₂ (20.70 mL). Then, pyridine (1.52 mL, 18.62 mmol) and DMAP (0.05 g, 0.41 mmol) were added at 0° C. The resulted mixture was stirred overnight at rt. Then, the reaction mixture was diluted with sat. bicarbonate solution and the aqueous phase was extracted with CH₂Cl₂. The combined organic layers were washed with brine, dried over sodium sulfate, and concentrated over vacuum. The obtained crude was purified by flash chromatography (cyclohexane/EtOAc; 0-60%) to afford the product as colorless oil (0.80 g, 51%). ¹H NMR (400 MHZ, DMSO-d₆) δ7.71-7.64 (m, 2H, m-tosyl), 7.47-7.38 (m, 2H, o-tosyl), 7.32-7.22 (m, 2H, H3, H5), 7.16 (t, J=7.8 Hz, 1H, H5′), 7.01-6.90 (m, 3H, H2, H4, H6), 6.84 (ddd, J=8.1, 2.3, 0.8 Hz, 1H, H4), 6.82-6.76 (m, 1H, H2′), 6.76-6.71 (m, 1H, H6′), 4.20 (t, J=6.4 Hz, 2H, CH₂, H1″), 3.22 (s, 3H, CH₃, N-Me), 2.82 (t, J=6.4 Hz, 2H, CH₂, H2″), 2.40 (s, 3H, CH₃, tosyl-Me).

### 3-((tert-butyldimethylsilyl) oxy)propan-1-amine (46)

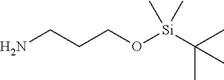

To a solution of 3-aminopropanol (2.00 g, 26.63 mmol) in CH₂Cl₂ (100.00 mL), Et₃N (4.10 mL, 29.29 mmol) was added. Followed by the addition of a solution of TBSCl (4.05 g, 26.63 mmol) in CH₂Cl₂ (33.10 mL). over 5 min. The reaction mixture was stirred magnetically overnight at ambient temperature. Then, the reaction was diluted with saturated bicarbonate solution and the organic layer was separated. The aqueous phase was extracted with CH₂Cl₂. The combine organic layers were washed with brine, and dried over sodium sulfate. The crude product was purified over silica to afford the product as colorless liquid (4.35 g, 84%). ¹H NMR (400 MHZ, DMSO-d₆) δ3.60 (t, J=6.3 Hz, 2H, CH₂, H3), 2.56 (t, J=6.8 Hz, 2H, CH₂, H1), 1.50 (p, J=6.5 Hz, 2H, CH₂, H2), 0.83 (s, 9H, CH₃, t-butyl), 0.00 (s, 6H, CH₃)

### 3-(2-((3-((tert-butyldimethylsilyl)oxy)propyl) amino) ethyl)-N-methyl-N-phenylaniline (47)

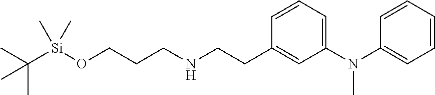 [SMILES: CN(c1ccccc1)c1cccc(CCNCCCO[Si](C)(C)C(C)(C)C)c1]

A solution of compound 45 (0.57 g, 1.49 mmol) in dry DMF (3.70 mL) was added a solution of amine 46 (0.43 g, 2.23 mmol) in dry DMF (3.70 mL). Then, CsCO₃ (1.06 g, 2.98 mmol) was added and the suspension was heated to 80° C. and stirred at this temperature for 6 h. The reaction was allowed to cool down to rt. Then, the organic solvent was removed, and the obtained residue was diluted with saturated bicarbonate solution. The aqueous phase was extracted with EtOAc. The combined organic layers were washed with brine, dried over sodium sulfate, and concentrated. The crude product was purified over silica (cyclohexane/EtOAc; 0-70-100%) to afford the product (0.25 g, 43%). ¹H NMR (400 MHZ, DMSO-d₆) δ7.28-7.21 (m, 2H, H3, H5), 7.17 (t, J=7.7 Hz, 1H, H5′), 6.99-6.94 (m, 2H, H2, H6), 6.90 (tt, J=7.5, 1.1 Hz, 1H, H4), 6.86-6.83 (m, 1H, H2′), 6.82 (ddd, J=8.1, 2.4, 1.0 Hz, 1H, H4′), 6.80-6.77 (m, 1H, H6′), 3.60 (t, J=6.3 Hz, 2H, CH₂, H1”), 3.23 (s, 3H, CH₃, N-Me), 2.70-2.64 (m, 2H, CH₂, H4″), 2.64-2.57 (m, 2H, CH₂, H5″), 2.57-2.51 (m, 2H, CH₂, H3″), 1.55 (p, J=6.6 Hz, 2H, CH₂, H2″), 0.84 (s, 9H, CH₃, t-butyl), 0.00 (s, 6H, CH₃)

### 3-((3-(methyl(phenyl)amino)phenethyl) amino)propan-1-ol(48)

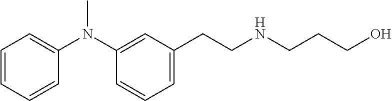 [SMILES: CN(c1ccccc1)c1cccc(CCNCCCO)c1]

Compound 47 (0.23 g, 0.57 mmol) was dissolved in THF (2.80 mL). Then, TBAF (0.31 mL, 1.13 mmol) was added dropwise. The reaction mixture was stirred overnight at ambient temperature. TLC(CH₂Cl₂/MeOH; 10%) indicated complete conversion. Then, the reaction mixture was concentrated under reduced pressure. The obtained residue was purified over silica (CH₂Cl₂/MeOH; 0-20%) to give the desired product 48 (0.14 g, 87%). ¹H NMR (400 MHZ, DMSO-d₆) δ7.30-7.23 (m, 2H, H3, H5), 7.19 (t, J=7.8 Hz, 1H, H5′), 7.01-6.96 (m, 2H, H2, H6), 6.92 (tt, J=7.5, 1.1 Hz, 1H, H4), 6.88-6.85 (m, 1H, H2′), 6.87-6.82 (m, 1H, H4′), 6.82-6.79 (m, 1H, H6′), 3.44 (t, J=6.3 Hz, 2H, CH₂, H1″), 3.25 (s, 3H, CH₃, N-Me), 2.75-2.68 (m, 2H, CH₂, 4″), 2.67-2.62 (m, 2H, CH₂, H5″), 2.59 (t, J=6.9 Hz, 2H, CH₂, H3″), 1.54 (p, J=6.6 Hz, 2H, CH₂, H2″).

### tert-butyl (3-hydroxypropyl)(3-(methyl(phenyl)amino)phenethyl)carbamate (49)

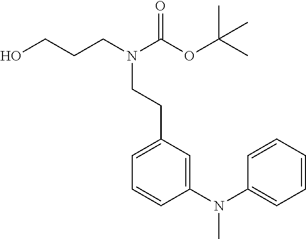 [SMILES: CN(c1ccccc1)c1cccc(CCN(CCCO)C(=O)OC(C)(C)C)c1]

To a solution of compound 48 (0.13 g, 0.47 mmol) in CH₂Cl₂ (2.30 mL), Et₃N (0.10 mL, 0.70 mmol) was added. Then, the solution was cooled down in an ice-bath. Afterwards, di-tert-butyl dicarbonate (0.12 mL, 0.51 mmol) was added dropwise to the cooled solution. The reaction mixture was stirred magnetically at rt until complete conversion was monitored by TLC (cyclohexane/EtOAc; 50%). After 5 h, the reaction was diluted with saturated bicarbonate solution and extracted with CH₂Cl₂. The combined organic layers were washed with brine, dried over sodium sulfate, and concentrated under reduced pressure. The obtained crude product was purified by flash chromatography (cyclohexane/EtOAc; 10-80%) which afforded the desired product (0.15 g, 85%). ¹H NMR (400 MHZ, DMSO-d₆) δ7.27 (m, 2H, H3, H5), 7.20 (dd, J=8.4, 7.5 Hz, 1H, H5′), 7.02-6.95 (m, 2H, H2, H6), 6.93 (t, J=7.2 Hz, 1H, H4), 6.88-6.81 (m, 2H. H2′, H4′), 6.81-6.75 (m, 1H, H6′), 4.41 (t, J=4.8 Hz, 1H, OH), 3.37 (q, J=6.4 Hz, 2H, CH₂, 1″), 3.34-3.28 (m, 2H, CH₂, H4″), 3.25 (s, 3H, CH₃, N-Me), 3.18-3.08 (m, 2H, CH₂, H3″), 2.73-2.65 (m, 2H, CH₂, H5″), 1.58 (p, J=6.4 Hz, 2H, CH₂, H2″), 1.42-1.26 (m, 9H, CH₃, t-butyl).

### tert-butyl (3-(methyl(phenyl)amino)phenethyl)(3-oxopropyl)carbamate (50)

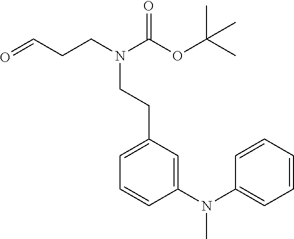 [SMILES: CN(c1ccccc1)c1cccc(CCN(CCC=O)C(=O)OC(C)(C)C)c1]

To a solution of alcohol 49 (0.09 g, 0.23 mmol) in dry DMSO (1.20 mL) was added Et₃N (0.07 mL, 0.47 mmol) at rt. The solution was vigorously stirred while a solution of SO₃ pyridine complex (0.08 g, 0.47 mmol) in dry DMSO (1.10 mL) was dropwise added. TLC (cyclohexane/EtOAc; 33%) indicated the formation of a new spot. After 1 h, the solution was quenched with water at 0° C. and extracted with CH₂Cl₂. The combined organic layers were dried over sodium sulfate and concentrated over vacuum. The crude product was purified over silica (cyclohexane/EtOAc; 10-60%) to afford the product as yellowish oil (0.05 g, 60%). ¹H NMR (400 MHZ, DMSO-d₆) δ9.64 (s, 1H, —CHO, H1″), 7.30-7.24 (m, 2H, H3, H5), 7.21 (dd, J=8.3, 7.6 Hz, 1H, H5′), 6.99 (d, J=7.7 Hz, 2H, H2, H6), 6.93 (t, J=7.3 Hz, 1H, H4), 6.89-6.81 (m, 2H, H2′, H4′), 6.82-6.75 (m, 1H, H6′), 3.43-3.35 (m, 2H, CH₂, H3″), 3.34-3.28 (m, 2H, CH₂, H4″), 3.25 (s, 3H, CH₃, N-Me), 2.72-2.65 (m, 2H, CH₂, H5″), 2.59 (td, J=6.7, 1.8 Hz, 2H, CH₂, H2″), 1.41-1.27 (m, 9H, CH₃, t-butyl).

### Preparation of (9H-fluoren-9-yl) methyl(3-oxopropyl)carbamate (9H-fluoren-9-yl) methyl(3-hydroxypropyl)carbamate (51)

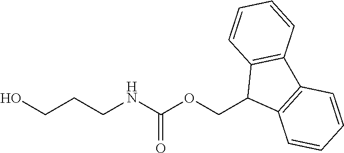 [SMILES: O=C(NCCCO)OCC1c2ccccc2-c2ccccc21]

3-Amino-propanol (1.24 mL, 1.22 mmol) was dissolved in CH₂Cl₂ (33.50 mL) and then cooled down in an ice-bath. To this cooled solution a solution of FmocCl (3.50 g, 13.39 mmol) in CH₂Cl₂ (33.50 mL) was added dropwise. The reaction mixture was allowed to warm up to rt and stirred at this temperature overnight. Then, the mixture was diluted with sat. bicarbonate solution. The organic phase was separated, and the aqueous phase was extracted three times with CH₂Cl₂. The combined organic layers were washed with brine, dried over sodium sulfate, and concentrated under reduced pressure. The obtained residue was purified over silica (cyclohexane/EtOAc; 0-100%) to afford the product as white solid (2.74 g, 69%). ¹H NMR (400 MHZ, DMSO-d₆) δ 7.90 (dd, J=0.8 Hz, 1H, H5, Fmoc), 7.88 (dd, J=1.2, 0.7 Hz, 1H, H4, Fmoc), 7.86 (dd, J=0.8 Hz, 1H, H1, Fmoc), 7.84 (dd, J=0.8 Hz, 1H, H8, Fmoc), 7.42 (td, J=7.4, 1.2 Hz, 2H, H2/7, Fmoc), 7.35 (td,)=7.4, 1.2 Hz, 2H, H3/6, Fmoc), 6.62 (t, J=4.8 Hz, 1H, NH), 6.29 (s, 2H, CH₂, Fmoc), 3.40 (t, J=6.3 Hz, 2H, CH₂, H1), 2.98 (q, J=6.5 Hz, 2H, CH₂, H3), 1.53 (p, J=6.5 Hz, 2H, CH₂, H2)

### (9H-fluoren-9-yl) methyl(3-oxopropyl)carbamate (52)

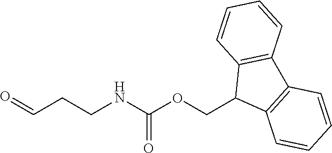 [SMILES: O=CCCNC(=O)OCC1c2ccccc2-c2ccccc21]

The alcohol 51 (0.93 g, 3.08 mmol) was dissolved in CH₂Cl₂ (15.40 mL) and cooled down to 0° C. in an ice-bath. Then, DMP (1.45 g, 3.39 mmol) was added, and the resulted mixture was stirred for 10 min at 0° C. The reaction mixture was allowed to warm up to rt and stirred magnetically for 4 h. TLC( ) indicated that the reaction was terminated. Then, the reaction was quenched with saturated bicarbonate solution and stirred for 10 min at ambient temperature. The organic layer was separated, and the aqueous phase was extracted with CH₂Cl₂. The combined organic layers were washed with brine, dried over sodium sulfate, and concentrated under reduced pressure. The crude product was purified by flash chromatography ( ) to afford the pure product was white solid (0.78 g, 86%). ¹H NMR (400 MHZ, DMSO-d₆) δ9.62 (t, J=1.6 Hz, 1H, H3, —CHO), 7.91-7.87 (m, 2H, H4/5, Fmoc), 7.67 (d, J=7.3 Hz, 2H, H1/8, Fmoc), 7.44-7.39 (m, 2H, H2/7, Fmoc), 7.37 (t, J=5.7 Hz, 1H, NH), 7.33 (td, J=7.4, 1.1 Hz, 2H, H3/6, Fmoc), 4.29 (s, 2H, CH₂, Fmoc), 3.27 (q, J=6.4 Hz, 2H, CH₂, H1), 2.56 (td, J=6.5, 1.5 Hz, 2H, CH₂, H2)

### Preparation of tert-butyl (4-ethynylphenethyl)(3-oxopropyl)carbamate N-(4-bromophenethyl)-3-((tert-butyldimethylsilyl)oxy)propan-1-amine (53)

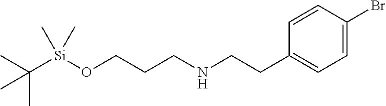 [SMILES: CC(C)(C)[Si](C)(C)OCCCNCCc1ccc(Br)cc1]

4-Bromophenyl-acetaldehyde (0.72 g, 3.58 mmol) was dissolved in dry MeOH (8.95 mL). Then, a solution of compound x (0.69 g, 3.58 mmol) in dry MeOH (8.95 mL) was added portion wise ade rt. The resulted mixture was stirred for 72 h at ambient temperature. The solution was cooled down to 0° C. before NaBH₄ (0.21 g, 5.37 mmol) was added portion wise. The solution was stirred for 6 h at rt. Then, water was added. The aqueous phase was extracted with EtOAc. The combined organic layers were dried over sodium sulfate and concentrated under reduced pressure. The obtained residue was purified over silica (cyclohexane/EtOAc; 20-100%) to afford the desired product as yellowish resin (0.36 g, 27%). ¹H NMR (400 MHZ, DMSO-d₆) δ7.49-7.42 (m, 2H, m-Ar), 7.21-7.14 (m, 2H, o-Ar), 3.65-3.57 (m, 4H, CH₂, H3′, H4′), 2.72-2.61 (m, 2H, CH₂, H5′), 2.57-2.52 (m, 2H, CH₂, H1′), 1.62-1.51 (m, 2H, CH₂, H2′), 0.85-0.83 (m, 9H, CH₃, t-butyl, OTBS), 0.01--0.01 (m, 6H, CH₃, Me, OTBS).

### tert-butyl (4-bromophenethyl)(3-((tert-butyldimethylsilyl)oxy)propyl)carbamate (54)

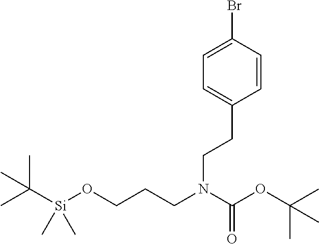 [SMILES: CC(C)(C)OC(=O)N(CCCO[Si](C)(C)C(C)(C)C)CCc1ccc(Br)cc1]

To a solution of 53 (0.36 g, 0.97 mmol) in CH₂Cl₂ (4.80 mL), Et₃N (0.20 mL, 1.45 mmol) was added. Then, the solution was cooled down in an ice-bath before di-tert-butyl decarbonate (0.25 mL, 1.07 mmol) was added portion wise. The reaction mixture was allowed to warm up to rt and stirred magnetically at this temperature until TLC(cyclohexane/EtOAc; 50%) monitored complete conversion. After 3 h, the reaction was diluted with sat. bicarbonate solution and extracted with CH₂Cl₂. The combined organic layers were washed with brine, dried over sodium sulfate, and concentrated over vacuum. The obtained residue was purified over silica (cyclohexane/EtOAc; 10-80%) to afford the product as yellowish resin (0.36 g, 79%). ¹H NMR (400 MHz, DMSO-d₆) δ7.56-7.41 (m, 2H, m-Ar), 7.19-7.08 (m, 2H, o-Ar), 3.58-3.47 (m, 2H, CH₂, H1′), 3.32-3.27 (m, 2H, CH₂, H4′), 3.20-3.05 (m, 2H, CH₂, H3′), 2.77-2.66 (m, 2H, CH₂, H5′), 1.65-1.54 (m, 2H, CH₂, H2′), 1.39-1.28 (m, 9H, CH₃, t-butyl, NBoc), 0.85-0.81 (m, 9H, CH₃, t-butyl, OTBS), 0.01--0.03 (m, 6H, CH₃, Me, OTBS).

### tert-butyl (3-((tert-butyldimethylsilyl)oxy)propyl)(4-((trimethylsilyl) ethynyl)phenethyl)carbamate (55)

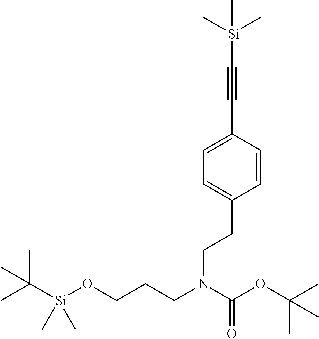 [SMILES: CC(C)(C)OC(=O)N(CCCO[Si](C)(C)C(C)(C)C)CCc1ccc(C#C[Si](C)(C)C)cc1]

A heat-dried two-necked round bottom flash was equipped with a stirring bar and charged with Na₂PdCl₄ (7.00 mg, 0.02 mmol), Cul (13.00 mg, 0.07 mmol), and PIntB (7.00 mg, 0.02 mmol) under nitrogen atmosphere. Afterwards, a solution of compound 54 in TEMEDA (3.50 mL) was added under nitrogen atmosphere. Then, the mixture was degassed before TMS-acetylene (0.22 mL, 1.47 mmol) was added. The reaction mixture was degassed and then heated to 80° C. The mixture was stirred overnight at 80° C. After 17 h, the reaction mixture was cooled down to ambient temperature. Then, water was added, and the mixture was extracted with EtOAc. The combined organic layers were washed with brine, dried over sodium suflate, and concentrated under vacuum. The obtained residue was purified over silica (cyclohexane/EtOAc; 0-100%) to afford the product as colorless oil (0.25 g, 75%). ¹H NMR (400 MHZ, DMSO-d₆) δ7.25-7.11 (m, 2H, m-Ar), 7.02-6.92 (m, 2H, o-Ar), 3.31 (t, J=6.1 Hz, 2H, CH₂, H1′), 3.13-3.06 (m, 2H, CH₂, H4′), 2.89-2.81 (m, 2H, CH₂, H3′), 2.58-2.51 (m, 2H, CH₂, H5′), 1.42-1.32 (m, 2H, CH₂, H2′), 1.22-1.09 (m, 9H, CH₃, t-butyl, NBoc), 0.64-0.60 (m, 9H, CH₃, t-butyl, OTBS), 0.01--0.01 (m, 9H, CH₃, CH₃, TMS), —0.21--0.23 (m, 6H, CH₃, Me, OTBS).

### tert-butyl (4-ethynylphenethyl)(3-hydroxypropyl)carbamate (56)

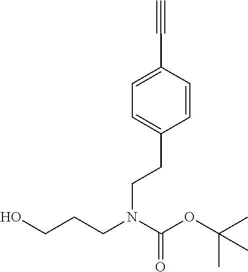 [SMILES: C#Cc1ccc(CCN(CCCO)C(=O)OC(C)(C)C)cc1]

Compound 55 (0.25 g, 0.51 mmol) was dissolved in THF (5.10 mL). Then, a 1 M TBAF (0.21 mL, 0.76 mmol) solution in THF (0.55 mL) was added portion wise. The reaction mixture was stirred at ambient temperature for 4 h. TLC(CH₂Cl₂/MeOH, 5%) indicated full conversion. The reaction mixture was diluted with CH₂Cl₂ and water. The organic layer was separated, and the aqueous phase was extracted with CH₂Cl₂. The combined organic layers were dried over sodium sulfate and concentrated over vacuum. The obtained residue was purified over silica (CH₂Cl₂/MeOH, 0-5%) to afford the desired product as colorless oil (0.14 g, 93%). ¹H NMR (400 MHZ, DMSO-d₆) δ 7.48-7.36 (m, 2H, m-Ar), 7.24-7.17 (m, 2H, o-Ar), 4.44-4.35 (m, 2H, OH, alkyne), 3.39-3.30 (m, 2H, CH₂, H1′), 3.22-3.07 (m, 2H, CH₂, H3′, H4′), 2.76 (t, J=7.4 Hz, 2H, CH₂, H5′), 1.58 (p, J=6.4 Hz, 2H, CH₂, H2′), 1.40-1.28 (m, 9H, CH₃, t-butyl, NBoc).

### tert-butyl (4-ethynylphenethyl)(3-oxopropyl)carbamate (57)

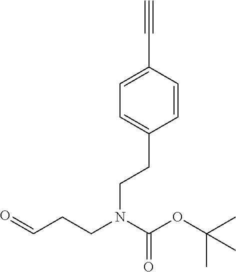 [SMILES: C#Cc1ccc(CCN(CCC=O)C(=O)OC(C)(C)C)cc1]

The alcohol 56 (0.06 g, 0.20 mmol) was dissolved in dry DMSO (2.00 mL). To this solution was added Et₃N (0.06 mL, 0.40 mmol). Then, a solution of sulfur trioxide pyridine complex in dry DMSO (2.00 mL) were added slowly to the vigorously stirred solution. After 2 h, the solution was quenched with water at 0° C. and the aqueous phase was extracted with CH₂Cl₂. The combined organic layers were dried over sodium sulfate and concentrated under reduced pressure. The crude product was purified by FC(cyclohexane/EtOAc; 10-60%) to afford the product as yellowish oil (0.04 g, 69%). ¹H NMR (400 MHZ, DMSO-d₆) δ9.66-9.63 (m, 1H, CHO), 7.50-7.37 (m, 2H, m-Ar), 7.27-7.18 (m, 2H, o-Ar), 4.39 (s, 1H, CH, alkyne), 3.44-3.32 (m, 2H, CH₂, H2′, H3′), 2.77 (t, J=7.3 Hz, 2H, CH₂, H4′), 2.65-2.58 (m, 2H, CH₂, H1′), 1.40-1.27 (m, 9H, CH₃, t-butyl, NBoc).

### Synthesis of tert-butyl (2-fluorophenethyl)(3-oxopropyl)carbamate 2-(2-fluorophenyl)acetaldehyde (58)

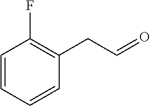

1.34 mL (10 mmol; 1.0 eq) of 2-fluorophenethyl alcohol were dissolved in 50 ml of DCM. At 0° C., 5090 mg (12 mmol; 1.2 eq) of dess-martin-periodinane were added in portions. The reaction solution was continuously stirred for 2.5 h while warming to RT. Then, 40 mL of a 1 m Na₂S203 solution were added to the reaction mixture and stirred for an additional 15 min. After addition of another 100 ml of DCM, the organic phase was washed with 80 mL each of a 5% NaHCO₃ solution and a saturated NaCl solution. The organic phase was dried over MgSO₄, filtered, and the solvent was removed under reduced pressure. Column chromatography on silica gel (cyclohexane/EtOAc 100:0% to 90:10%) gave 663 mg (4.80 mmol; 48%) of the title compound in the form of a colorless oil. 1H-NMR: (400 MHZ, DMSO-d₆) δ=9.72-9.69 (m, 1H), 7.38-7.33 (m, 1H), 7.32-7.29 (m, 1H), 7.23-7.19 (m, 1H), 7.19-7.16 (m, 1H), 3.88-3.84 (m, 2H) ppm. 1H-NMR: (400 MHZ, DMSO-d₆) δ=7.32-7.28 (m, 1H), 7.26-7.21 (m, 2H), 7.14-7.09 (m, 2H), 3.43 (t, 3 J=6.3 Hz, 2H), 2.76-2.67 (m, 4H), 2.58 (t, 3)=6.8 Hz, 2H), 1.57-1.50 (m, 2H) ppm.

### 3-((2-fluorophenethyl) amino)propan-1-ol (59)

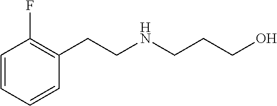 [SMILES: OCCCNCCc1ccccc1F]

663 mg (4.80 mmol; 1.0 eq) of 2-(2-fluorophenyl)acetaldehyde were dissolved in 20 ml of methanol and 0.37 mL (4.80 mmol; 1.0 eq) of 3-amino-1-propanol were added. The reaction solution was stirred at RT for 16 h under N₂ atmosphere. Finally, at 0° C., 272 mg (7.20 mmol; 1.5 eq) NaBH₄ were added. After another 2 h of stirring, during which the solution warmed to RT, it was quenched with 50 ml of a saturated NaCl solution. Extraction was performed with EtOAc (3×60 mL). The combined organic phases were dried over MgSO₄, filtered, and the solvent was removed under reduced pressure. After purification by column chromatography on silica gel (DCM/MeOH 100:0% to 80:20%), 300 mg (1.52 mmol; 32%) of the title compound was obtained in the form of a colorless oil.

### tert-butyl (2-fluorophenethyl)(3-hydroxypropyl)carbamate (60)

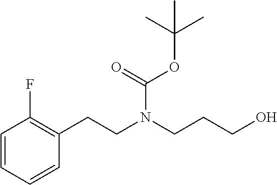 [SMILES: CC(C)(C)OC(=O)N(CCCO)CCc1ccccc1F]

300 mg (1.52 mmol; 1.0 eq)₃-((2-fluorophenethyl) amino)propan-1-ol were dissolved in 30 ml DCM and 0.32 mL Et₃N (2.28 mmol; 1.5 eq) and 365 mg (1.67 mmol; 1.1 eq) Boc₂O were added at 0° C. The solution was warmed to RT and stirred for 16 h. The solvent was then removed and the resulting residue adsorbed on silica gel. Column chromatography (cyclohexane/EtOAc 100:0% to 50:50%) gave 411 mg (1.38 mmol; 91%) of the title compound in the form of a colorless oil. 1H-NMR: (400 MHZ, DMSO-d₆) δ=7.30-7.21 (m, 2H), 7.18-7.09 (m, 2H), 4.41 (t, 3)=5.1 Hz, 1H), 3.41-3.34 (m, 4H), 3.22-3.03 (m, 2H), 2.79 (t, 3)=7.1 Hz), 1.63-1.54 (m, 2H), 1.35 and 1.28 (s, 9H) ppm.

### tert-butyl (2-fluorophenethyl)(3-oxopropyl)carbamate (61)

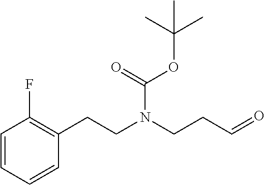 [SMILES: CC(C)(C)OC(=O)N(CCC=O)CCc1ccccc1F]

411 mg (1.38 mmol; 1.0 eq) tert-butyl (2-fluorophenethyl)(3-hydroxypropyl)carbamate were dissolved in 5 mL DMSO, to which were added 0.38 mL (2.76 mmol; 2.0 eq) Et₃N. Then, 439 mg (2.76 mmol; 2.0 eq) of pyridine sulfur trioxide were added to the reaction solution in portions. Stirring was continued for 2 h at RT before the reaction was stopped with addition of 20 mL dist. H₂O. After extraction with DCM (3×60 mL), the combined organic phases were dried over MgSO₄ and filtered. The solvent was removed under reduced pressure and the residue was purified by column chromatography on silica gel (cyclohexane/EtOAc 100:0% to 70:30%). 282 mg (0.95 mmol; 69%) of the title compound was obtained as a colorless oil. 1H-NMR: (400 MHZ, DMSO-d₆) δ=9.64 (t, 3) =1.8 Hz), 7.31-7.21 (m, 2H), 7.18-7.09 (m, 2H), 3.49-3.30 (m, 4H), 2.79 (t, 3 J=7.0 Hz, 2H), 2.61 (td, 3)=6.6, 1.5 Hz), 1.35 and 1.25 (s, 9H) ppm.

### Synthesis of tert-butyl (4-fluorophenethyl)(3-oxopropyl)carbamate 2-(4-fluorophenyl)acetaldehyde (62)

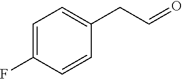

1.35 mL (10.78 mmol; 1.0 eq) of 4-fluorophenethyl alcohol were dissolved in 50 mL of DCM. At 0° C., 5480 mg (12.92 mmol; 1.2 eq) of dess-martin-periodinane were added in portions. The reaction solution was continuously stirred for 2 h while warming to RT. Then, 40 ml of a 1 M Na₂S203 solution were added to the reaction mixture and stirred for an additional 15 min. After addition of another 100 mL of DCM, the organic phase was washed with 80 mL each of a 5% NaHCO₃ solution and a saturated NaCl solution. The organic phase was dried over MgSO₄, filtered, and the solvent was removed under reduced pressure. Column chromatography on silica gel (cyclohexane/EtOAc 100:0% to 90:10%) gave 1140 mg (8.24 mmol; 76%) of the title compound in the form of a colorless oil.

### 3-((4-fluorophenethyl) amino)propan-1-ol (63)

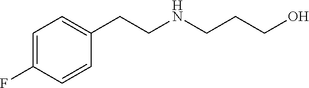 [SMILES: OCCCNCCc1ccc(F)cc1]

1140 mg (8.24 mmol; 1.0 eq) of 2-(4-fluorophenyl)acetaldehyde were dissolved in 50 ml of methanol and 0.63 mL of 3-amino-1-propanol (8.24 mmol; 1.0 eq) were added. The reaction solution was stirred at RT for 14 h under N₂ atmosphere. Finally, at 0° C., 467 mg (12.36 mmol; 1.5 eq) NaBH₄ were added. After another 3.5 h of stirring, during which the solution warmed to RT, it was quenched with 80 ml of a saturated NaCl solution. Extraction was performed with EtOAc (3×90 mL). The combined organic phases were dried over MgSO₄, filtered, and the solvent was removed under reduced pressure. After purification by column chromatography on silica gel (DCM/MeOH 100:0% to 80:20%), 490 mg (2.48 mmol; 30%) of the title compound was obtained in the form of a pale yellow oil.

### tert-butyl (4-fluorophenethyl)(3-hydroxypropyl)carbamate (64)

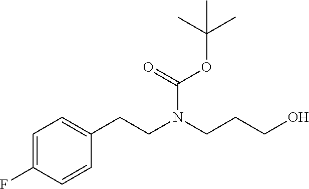 [SMILES: CC(C)(C)OC(=O)N(CCCO)CCc1ccc(F)cc1]

490 mg (2.48 mmol; 1.0 eq)₃-((4-fluorophenethyl) amino)propan-1-ol were dissolved in 30 ml DCM and 0.50 mL Et₃N (3.60 mmol; 1.5 eq) and 595 mg (2.73 mmol; 1.1 eq) Boc₂O were added at 0° C. The solution was warmed to RT and stirred for 14 h. The solvent was then removed and the resulting residue adsorbed on silica gel. Column chromatography (cyclohexane/EtOAc 100:0% to 50:50%) gave 640 mg (2.15 mmol; 87%) of the title compound in the form of a colorless oil.

### tert-butyl (4-fluorophenethyl)(3-oxopropyl)carbamate (65)

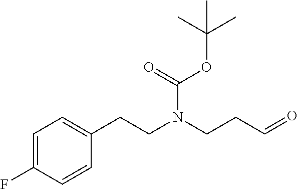 [SMILES: CC(C)(C)OC(=O)N(CCC=O)CCc1ccc(F)cc1]

640 mg (2.15 mmol; 1.0 eq) tert-butyl (4-fluorophenethyl)(3-hydroxypropyl)carbamate were dissolved in 7 mL DMSO, to which were added 0.60 mL (4.30 mmol; 2.0 eq) Et₃N. Then, 684 mg (4.30 mmol; 2.0 eq) of pyridine sulfur trioxide were added to the reaction solution in portions. Stirring was continued for 2 h at RT before the reaction was stopped with addition of 20 mL dist. H₂O. After extraction with DCM (3×60 mL), the combined organic phases were dried over MgSO₄ and filtered. The solvent was removed under reduced pressure and the residue was purified by column chromatography on silica gel (cyclohexane/EtOAc 100:0% to 40:60%). 437 mg (1.48 mmol; 62%) of the title compound was obtained as a colorless oil.

### Synthesis of tert-butyl (4-fluorobenzyl)(3-oxopropyl)carbamate 3-((4-fluorobenzyl) amino)propan-1-ol (66)

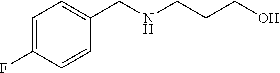 [SMILES: OCCCNCc1ccc(F)cc1]

2482 mg (20.00 mmol; 1.0 eq) of 4-fluorobenzaldehyde were dissolved in 30 ml of methanol and 1.53 mL (20.00 mmol; 1.0 eq) of 3-amino-1-propanol were added. The reaction solution was stirred at RT for 20 h under N₂ atmosphere. Finally, at 0° C., 1135 mg (30.00 mmol; 1.5 eq) NaBH₄ were added. After another 2 h of stirring, during which the solution warmed to RT, it was quenched with 50 ml of a saturated NaCl solution. Extraction was performed with EtOAc (3×60 mL). The combined organic phases were dried over MgSO₄, filtered, and the solvent was removed under reduced pressure. After purification by column chromatography on silica gel (DCM/MeOH 100:0% to 80:20%), 2163 mg (11.80 mmol; 59%) of the title compound was obtained in the form of a colorless oil.

### tert-butyl (4-fluorobenzyl)(3-hydroxypropyl)carbamate (67)

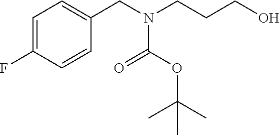 [SMILES: CC(C)(C)OC(=O)N(CCCO)Cc1ccc(F)cc1]

2163 mg (11.80 mmol; 1.0 eq)₃-((4-fluorobenzyl) amino)propan-1-ol were dissolved in 100 ml DCM and 2.47 mL Et₃N (17.70 mmol; 1.5 eq) and 2832 mg (12.98 mmol; 1.1 eq) Boc₂O were added at 0° C. The solution was warmed to RT and stirred for 13.5 h. The solvent was then removed and the resulting residue adsorbed on silica gel. Column chromatography (cyclohexane/EtOAc 100:0% to 50:50%) gave 3087 mg (10.89 mmol; 92%) of the title compound in the form of a colorless oil.

### tert-butyl (4-fluorobenzyl)(3-oxopropyl)carbamate (68)

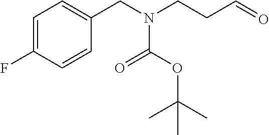 [SMILES: CC(C)(C)OC(=O)N(CCC=O)Cc1ccc(F)cc1]

850 mg (3.00 mmol; 1.0 eq)₃-((4-fluorobenzyl) amino)propan-1-ol were dissolved in 5 mL DMSO, to which was added 0.83 mL (6.00 mmol; 2.0 eq) Et₃N. Then, 955 mg (6.00 mmol; 2.0 eq) of pyridine sulfur trioxide were added to the reaction solution in portions. Stirring was continued for 3.4 h at RT before the reaction was stopped with addition of 20 mL dist. H₂O. After extraction with DCM (5×60 mL), the combined organic phases were dried over MgSO₄ and filtered. The solvent was removed under reduced pressure and the residue was purified by column chromatography on silica gel (cyclohexane/EtOAc 100:0% to 40:60%). 614 mg (2.18 mmol; 73%) of the title compound was obtained as a colorless oil.

### Synthesis of 2-(3-fluorophenyl)acetaldehyde 2-(3-fluorophenyl)acetaldehyde (69)

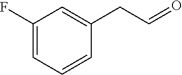

To a cooled solution of 3-fluorophenetyl alcohol (1.00 g, 7.06 mmol) in DCM (35.30 mL) DMP (3.33 g, 7.77 mmol) was added. After 10 min, the mixture was allowed to warm up to rt and stirred for 3 h. The mixture was quenched with saturated bicarbonate solution and stirred for 10 min. The organic layer was separate. The aqueous phase was extracted with DCM. The combined organic layers were washed with brine, dried over sodium sulfate, and concentrated. The crude product was purified over silica (cyclohexane/EtOAc; 0-10%) to afford the pure product as colorless oil (0.36 g, 37%).

### Synthesis of tert-butyl (3-oxopropyl)(4-(trifluoromethyl)phenethyl)carbamate 3-((4-(trifluoromethyl)phenethyl) amino)propan-1-ol (70)

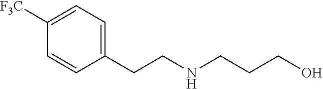 [SMILES: OCCCNCCc1ccc(C(F)(F)F)cc1]

0.75 ml (4.94 mmol; 1.00 eq) of 2-(4-(trifluoromethyl)phenyl)acetaldehyde were dissolved in 20 ml of Dichloromethane and 2515 mg (5.93 mmol; 1.20 eq) of Dess-Martin-periodinane were added in portions at 0° C. The reaction solution was continuously stirred for 6 h while warming to RT. Afterwards the solvent was removed under reduced pressure and the crude was adsorbed on silica. Column chromatography (Cyclohexane/EtOAc 100:0% to 80:20%) yielded to 440 mg (2.34 mmol; 24%) of the corresponding aldehyde, which was used for further reaction without characterization. Therefore, the aldehyde was dissolved in 20 ml of MeOH and 176 mg (2.34 mmol; 1.00 eq referred to the Aldehyde) of Aminopropanol were added. The reaction mixture was stirred over night at RT and then 133 mg (3.51 mmol; 1.50 eq) of NaBH₄ were added at 0° C. The reaction was warmed to RT. After 3 h the solvent was removed under reduced pressure. Column chromatography resulted in 260 mg (1.05 mmol; 30%) of the title compound in form of a colorless oil. 1H-NMR: (400 MHZ, DMSO-d₆) δ=7.70-7.60 (m, 2H), 7.49-7.41 (m, 2H), 3.44 (t, J=6.3 Hz, 1H), 2.85-2.76 (m, 4H), 2.63 (t, J=6.9 Hz, 2H), 1.59-1.53 (m, 2H) ppm; signal of two Protons overlayed by the H₂O-peak. APCI-MS (+) m/z for C₁₂H₁₆F₃NO: calc.: 247.26; found: 248.2.

### tert-butyl (3-hydroxypropyl)(4-(trifluoromethyl)phenethyl)carbamate (71)

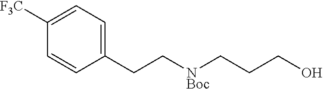 [SMILES: CC(C)(C)OC(=O)N(CCCO)CCc1ccc(C(F)(F)F)cc1]

260 mg (1.25 mmol; 1.00 eq) 3-((4-(trifluoromethyl)phenethyl) amino)propan-1-ol were dissolved in 30 ml of DCM. At 0° C. 0.22 mL (1.58 mmol; 1.50 eq) of Et₃N and 253 mg (1.16 mmol; 1.10 eq) of Boc₂O were added. The solution was warmed to RT and stirred for 20 h. The solvent was then removed and the resulting residue was adsorbed on silica gel. Column chromatography (Cyclohexane/EtOAc 100:0% to 50:50%) gave 340 mg (0.98 mmol; 78%) of the title compound in the form of a colorless oil. TLC: R^{f}=0.50 (Cyclohexane/EtOAc 1:1). 1H-NMR: (400 MHZ, DMSO-d₆)}=7.70-7.60 (m, 2H), 7.46-7.38 (m, 2H), 4.41 (t, J=5.0 Hz, 1H), 3.40-3.35 (m, 4H), 3.24-3.11 (m, 2H), 2.85 (t, J=7.2 Hz, 2H), 1.63-1.56 (m, 2H), 1.35 and 1.26 (s, 9H) ppm.

### tert-butyl (3-oxopropyl)(4-(trifluoromethyl)phenethyl)carbamate (72)

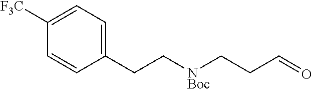 [SMILES: CC(C)(C)OC(=O)N(CCC=O)CCc1ccc(C(F)(F)F)cc1]

340 mg (0.98 mmol; 1.00 eq) of tert-butyl (3-hydroxypropyl)(4-(trifluoromethyl)phenethyl)carbamate were dissolved in 7 mL of DMSO. 0.27 ml (1.96 mmol; 2.00 eq) of Et₃N and 312 mg (1.96 mmol; 2.00 eq) of pyridine sulfur trioxide were added to the. Stirring was continued for 3 h at RT before the reaction was stopped with addition of 20 mL dist. H₂O. Extraction with DCM (3×60 mL) followed. The combined organic phases were dried over MgSO₄ and filtered. The solvent was removed under reduced pressure and the residue was purified by column chromatography on silica gel (Cyclohexane/EtOAc 100:0% to 40:60%). 235 mg (0.68 mmol; 69%) of the title compound were obtained as a colorless oil. TLC: R^{f}=0.71 (Cyclohexane/EtOAc 1:1). 1H-NMR: (400 MHZ, DMSO-d₆) δ=9.65 (s, 1H), 7.69-7.58 (m, 2H), 7.49-7.36 (m, 2H), 3.46-3.36 (m, 4H), 2.84 (t, J=7.2 Hz, 2H), 2.65-2.58 (m, 2H), 1.35 and 1.26 (s, 9H) ppm.

### Synthesis of tert-butyl (2,4-difluorophenethyl)(3-oxopropyl)carbamate

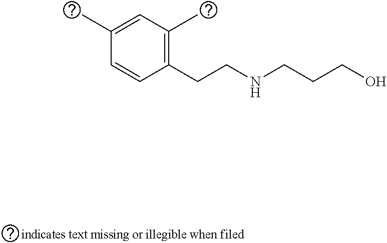

3-((2,4-difluorophenethyl) amino)propan-1-ol (73)

1400 mg (8.97 mmol; 1.0 eq) of 2-(2,4-difluorophenyl)acetaldehyde were dissolved in 20 ml of methanol and 0.37 mL (4.80 mmol; 1.0 eq) of 3-amino-1-propanol were added. The reaction solution was stirred at RT overnight under N₂ atmosphere. Finally, at 0° C., 512 mg (13.45 mmol; 1.5 eq) of NaBH₄ were added. After another 2 h of stirring, during which the solution was warmed to RT, it was quenched with 50 ml of a saturated NaCl solution. Extraction was performed with EtOAc (3×60 mL). The combined organic phases were dried over MgSO₄, filtered, and the solvent was removed under reduced pressure. After purification by column chromatography on silica gel (DCM/MeOH 100:0% to 90:10%), 738 mg (3.74 mmol; 42%) of the title compound were obtained in the form of a colorless oil. TLC: R^{f}=0.09 (DCM/MeOH 10:1). 1H-NMR: (400 MHZ, DMSO-d₆) δ=7.37-7.31 (m, 1H), 7.17-7.12 (m, 1H), 7.02-6.97 (m, 1H), 3.43 (t, J=6.3 Hz, 2H), 2.72-2.66 (m, 4H), 2.56 (t, J=6.8 Hz, 2H), 1.56-1.49 (m, 2H) ppm; signal of one Proton overlayed by the H₂O-peak.

### tert-butyl(2 4-diflunronhenethyl)/3-hydroxypropyl)carbamate (74)

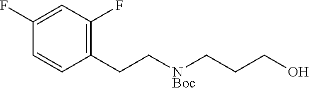 [SMILES: CC(C)(C)OC(=O)N(CCCO)CCc1ccc(F)cc1F]

514 mg (2.38 mmol; 1.0 eq) of 3-((2,4-difluorophenethyl) amino)propan-1-ol were dissolved in 30 ml of DCM and 0.49 mL (3.57 mmol; 1.5 eq) of Et₃N and 571 mg (2.62 mmol; 1.1 eq) of Boc₂O were added at 0° C. The solution was warmed to RT and stirred overnight. The solvent was then removed and the resulting residue adsorbed on silica gel. Column chromatography (Cyclohexane/EtOAc 95:5% to 50:50%) gave 642 mg (2.04 mmol; 86%) of the title compound in the form of a colorless oil. 1H-NMR: (400 MHZ, DMSO-d₆) δ=7.33-7.26 (m, 1H), 7.23-7.13 (m, 1H), 7.06-6.97 (m, 1H), 4.42 (t, J=5.1 Hz, 1H), 3.39-3.31 (m, 4H), 3.21-3.05 (m, 2H), 2.76 (t, J=7.0 Hz, 2H), 1.61-1.54 (m, 2H), 1.34 and 1.27 (s, 9H) ppm.

### tert-butyl (2.4-difluorophenethyl)(3-oxopropyl)carbamate (75)

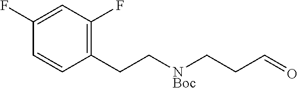 [SMILES: CC(C)(C)OC(=O)N(CCC=O)CCc1ccc(F)cc1F]

990 mg (3.18 mmol; 1.0 eq) of tert-butyl (2,4-difluorophenethyl)(3-hydroxypropyl)carbamate were dissolved in 7 mL of DMSO, to which were added 0.88 mL (6.36 mmol; 2.0 eq) of Et₃N. Then, 1012 mg (6.36 mmol; 2.0 eq) of pyridine sulfur trioxide were added to the reaction solution, which was stirred for another 3 h at RT. After extraction with DCM (3×60 mL), the combined organic phases were dried over MgSO₄ and filtered. The solvent was removed under reduced pressure and the residue was purified by column chromatography on silica gel (Cyclohexane/EtOAc 100:0% to 40:60%). 554 mg (1.79 mmol; 56%) of the title compound were obtained in form of a colorless oil. TLC: R^{f}=0.33 (Cyclohexane/EtOAc 3:1). 1H-NMR: (400 MHZ, DMSO-d₆) δ=9.64 (t, J=1.8 Hz, 1H), 7.34-7.28 (m, 1H), 7.23-7.11 (m, 1H), 7.08-6.97 (m, 1H), 3.44-3.34 (m, 4H), 2.77 (t, J=6.9 Hz, 2H), 2.61 (td, J=6.7, 1.9 Hz, 2H), 1.33 and 1.25 (s, 9H) ppm.

### Amine Sidechains

### Preparation of 1-phenethylpiperidin-4-amine

### tert-butyl (1-phenethylpiperidin-4-yl)carbamate (76)

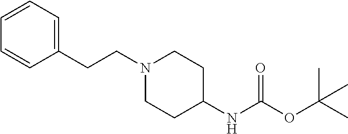 [SMILES: CC(C)(C)OC(=O)NC1CCN(CCc2ccccc2)CC1]

To a mixture of phenylacetaldeyde (0.50 g, 4.12 mmol) in dry MeOH (4.10 mL) was added dropwise as solution of tert-butyl piperidin-4-ylcarbamate (0.83 mL, 4.12 mmol) in dry MeOH (4.10 mL). The solution was stirred overnight at ambient temperature. Then, the reaction mixture was cooled down to 0° C. Afterwards, NaBH₄ (0.24 g, 6.18 mmol)) was added portion wise. After complete conversion the reaction was concentrated under reduced pressure. The obtained residue was diluted with water and extracted with EtOAc. The combined organic layers were washed with brine, dried over sodium sulfate, and concentrated. The crude product was purified by flash chromatography (CH₂Cl₂/MeOH; 0-10%) to afford the product as colorless oil (0.31 g; 24%).

### 1-phenethylpiperidin-4-amine (77)

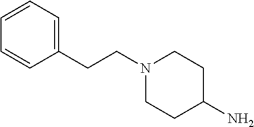 [SMILES: NC1CCN(CCc2ccccc2)CC1]

Compound 76 (0.30 g, 0.98 mmol) was dissolved in dry CH₂Cl₂ (5.90 mL). Then, a 6M HCl solution in iPrOH (4.00 mL) was added stirred for 3 h at rt. The solution was evaporated, and the obtained residue was diluted with 10% NaOH solution. The aqueous phase was extracted with EtOAc. The combined organic layers were dried over sodium sulfate and concentrated under vacuum to afford the pure product (0.17 g, 84%).

### Preparation of tert-butyl (3-aminopropyl)(2-(naphthalen-2-yl)ethyl)carbamate

### tert-butyl (3-azidopropyl)(2-(naphthalen-2-yl)ethyl)carbamate (70h)

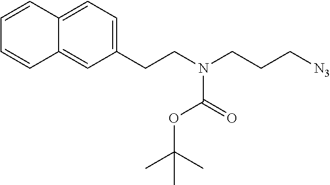 [SMILES: CC(C)(C)OC(=O)N(CCCN=[N+]=[N-])CCc1ccc2ccccc2c1]

To a stirred solution of tert-butyl (3-hydroxypropyl)(2-(naphthalen-2-yl)ethyl)carbamate (0.30 g, 0.90 mmol) in CH₂Cl₂ (4.50 mL) Et₃N (0.19 mL; 1.35 mmol) and MsCl (0.09 mL, 1.08 mmol) were added at 0° C. under nitrogen atmosphere. After 1h stirring at rt, the reaction was diluted with brine. The organic layer was separated, and the aqueous phase was extracted with CH₂Cl₂. The combined organic layers were dried over sodium sulfate and concentrated over vacuum to complete dryness. The mesylated product was dissolved in dry DMF (4.50 mL). To this solution NaN₃ (0.18 g, 2.71 mmol) was added and heated to 60° C. After 5 h, TLC(cyclohexane/EtOAc; 50%) monitored full consumption. Then, the organic solvent was removed under reduced pressure. Water was added and the aqueous phase was extracted with EtOAc. The combined organic layers were washed with brine, dried over sodium sulfate, and concentrated over vacuum. The obtained residue was purified over silica (cyclohexane/EtOAc; 0-50%) to afford the pure product (0.15, 45%).

### tert-butyl (3-aminopropyl)(2-(naphthalen-2-yl)ethyl)carbamate (71h)

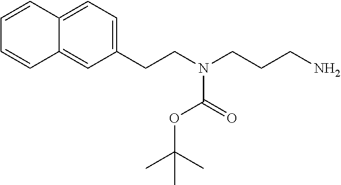 [SMILES: CC(C)(C)OC(=O)N(CCCN)CCc1ccc2ccccc2c1]

To a solution of 70h (0.14 g, 0.39 mmol) in EtOH (3.90 mL) palladium on activated charcoal moistened with water (0.08 g, 0.04 mmol) was added under nitrogen atmosphere at rt. Then, the reaction mixture was purged with hydrogen and stirred for 2 h at rt. The reaction progress was monitored by TLC(CH₂Cl₂/MeOH; 1%). Then, the reaction mixture was purged with nitrogen and the catalyst was filtered off. The filtrate was concentrated under reduced pressure and the obtained crude product was purified over silica (CH₂Cl₂/MeOH; 0-10%) to afford the product as colorless oil (0.12 g, 95%).

### Preparation of N₁-(4-(4-chlorophenoxy)-3-fluorophenethyl)propane-1,3-diamine 4-(4-chlorophenoxy)-3-fluorobenzaldehyde (78)

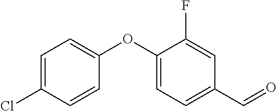 [SMILES: O=Cc1ccc(Oc2ccc(Cl)cc2)c(F)c1]

A suspension of 3,4-difluorobenzaldehyde (1.4 g, 10 mmol), 4-chlorophenol (1.3 g, 10 mmol), K₂CO₃ (2.8 g, 20 mmol) in DMF (15 mL) was stirred at 100° C. for 2 hrs. The mixture was diluted with EA (200 mL) and washed with H₂O (100 ml×2). The organic layer was dried over Na₂SO₄ and concentrated. The crude was purified by silica gel column (PE/EA=10/1) to give 4-(4-chlorophenoxy)-3-fluorobenzaldehyde (2.0 g, 80% yield) as a brown oil. MS Calc.: 250.0; MS Found: 251.2 [M+H]⁺.

### (E)-1-(4-chlorophenoxy)-2-fluoro-4-(2-methoxyvinyl)benzene (79)

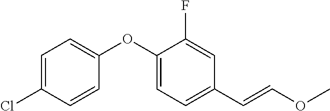 [SMILES: CO/C=C/c1ccc(Oc2ccc(Cl)cc2)c(F)c1]

To a solution of (methoxymethyl) triphenylphosphonium chloride (2.7 g, 8 mmol) in THF (20 mL) was added t-BuOK (900 mg, 8 mmol) at 0° C., and the solution was stirred at r. t. for 20 min. 4-(4-chlorophenoxy)-3-fluorobenzaldehyde (3, 500 mg, 2 mmol) was added and the solution was reacted for 1 hr. The reaction solution was diluted with EA (100 mL) and filtered. The filtrate was concentrated. The crude was purified by silica gel column (PE/EA=20/1) to give (E)-1-(4-chlorophenoxy)-2-fluoro-4-(2-methoxyvinyl)benzene (200 mg, Y: 36%) as a brown oil. MS Calc.: 278.1; MS Found: 279.2 [M+H]⁺.

### 2-(4-(4-chlorophenoxy)-3-fluorophenyl)acetaldehyde (80)

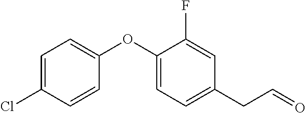 [SMILES: O=CCc1ccc(Oc2ccc(Cl)cc2)c(F)c1]

To a solution of (E)-1-(4-chlorophenoxy)-2-fluoro-4-(2-methoxyvinyl)benzene (200 mg, 0.82 mmol) in THF (5 mL) was added con. HCl (3 mL), and the solution was stirred at 70° C. for 1 hr. The reaction solution was diluted with EA (100 mL) and washed with H₂O (100 mL×2). The organic phase was dried over Na₂SO₄ and concentrated to give 2-(4-(4-chlorophenoxy)-3-fluorophenyl)acetaldehyde (200 mg, crude) as a brown oil.

### M-(4-(4-chlorophenoxy)-3-fluorophenethyl)propane-1,3-diamine (81)

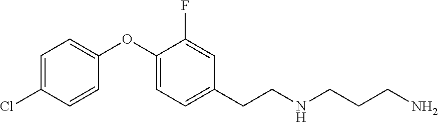 [SMILES: NCCCNCCc1ccc(Oc2ccc(Cl)cc2)c(F)c1]

A solution of 2-(4-(4-chlorophenoxy)-3-fluorophenyl)acetaldehyde (200 mg, 0.82 mmol) and propane-1,3-diamine (607 mg, 8.2 mmol) in MeOH (5 mL) was stirred at r. t. for 20 min. Then NaBH₄ (47 mg, 1.2 mmol) was added at 0° C., and the solution was stirred at r. t. for 1 hr. The reaction solution was diluted with EA (100 mL) and washed with H₂O (100 ml×3). The organic phase was dried over Na₂SO₄ and concentrated to give N₁-(4-(4-chlorophenoxy)-3-fluorophenethyl)propane-1,3-diamine (250 mg, crude) as a brown oil. MS Calc.: 322.0; MS Found: 323.3 [M+H]⁺.

### Preparation of N₁-methyl-N₃-(3-phenoxyphenethyl)propane-1,3-diamine tert-butyl methyl(3-((3-phenoxyphenethyl) amino)propyl)carbamate (82)

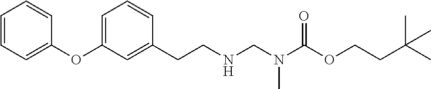 [SMILES: CN(CNCCc1cccc(Oc2ccccc2)c1)C(=O)OCCC(C)(C)C]

To a solution of tert-butyl (3-aminopropyl)(methyl)carbamate (376 mg; 2 mmol) in MeOH (5 mL) at 0° C. was added 2-(3-phenoxyphenyl)acetaldehyde (106 mg; 0.5 mmol) in small portions. After the addition, sodium borohydride (19 mg, 0.5 mmol) was added slowly in small portions at 0° C. The reaction was allowed to warm to room temperature overnight with stirring. The reaction was quenched with little water and concentrated to dryness. The crude was purified by reverse phase flash (MeCN/H₂O) to afford tert-butyl methyl(3-((3-phenoxyphenethyl) amino)propyl)carbamate (100 mg, 74% yield) as a yellow oil. MS Calc.: 385.2; MS Found: 386.2 [M+H]⁺.

### M-methyl-N₃-(3-phenoxyphenethyl)propane-1,3-diamine (83)

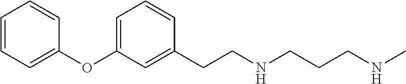 [SMILES: CNCCCNCCc1cccc(Oc2ccccc2)c1]

A solution of tert-butyl methyl(3-((3-phenoxyphenethyl) amino)propyl)carbamate (300 mg) in MeOH/HCl (5 mL) was stirred at r. t. for 2 hrs, followed by concentration to afford N₁-methyl-N₃-(3-phenoxyphenethyl)propane-1,3-diamine (crude) as a yellow oil.

### Preparation of N₁-(4-(4-chlorophenoxy)-3-fluorobenzyl)propane-1,3-diamine N-(4-(4-chlorophenoxy)-3-fluorobenzyl)propane-1,3-diamine (84)

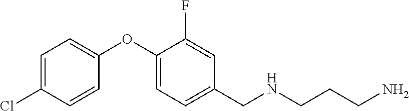 [SMILES: NCCCNCc1ccc(Oc2ccc(Cl)cc2)c(F)c1]

A solution of 4-(4-chlorophenoxy)-3-fluorobenzaldehyde (500 mg, 2 mmol), 1,3-diaminopropane (740 mg, 10 mmol) in MeOH (5 mL) was stirred at r. t. for 20 min. Then NaBH₄ (114 mg, 3 mmol) was added at 0° C., and the solution was stirred at r. t. for 1 h r. The reaction mixture was diluted with EA (100 mL). The organic phase was washed with H₂O (100 ml×3), dried over Na₂SO₄ and concentrated to give N₁-(4-(4-chlorophenoxy)-3-fluorobenzyl)propane-1,3-diamine (200 mg, crude) as a brown oil. MS Calc.: 308.1; MS Found: 309.3 [M+H]⁺.

### Preparation of N₁-(3-phenoxybenzyl)propane-1,3-diamine M-(3-phenoxybenzyl)propane-1,3-diamine (85)

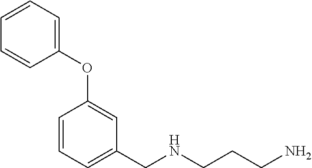 [SMILES: NCCCNCc1cccc(Oc2ccccc2)c1]

To a solution of 1,3-diaminopropane (150 mg; 2 mmol) in MeOH (5 mL) at 0° C. was added 3-(phenoxy)benzaldehyde (100 mg; 0.5 mmol) in small portions. After the addition, sodium borohydride (19 mg, 0.5 mmol) was added slowly in small portions at 0° C. The reaction was allowed to warm to room temperature overnight with stirring. The reaction was quenched with little water and concentrated to dryness. The crude was purified by reverse phase flash (MeCN/H₂O) to afford N₁-(3-phenoxybenzyl)propane-1,3-diamine (100 mg, 77% yield) as a yellow oil. MS Calc.: 256.2; MS Found: 257.3 [M+H]⁺.

### Preparation of N₁-(3-phenoxybenzyl)ethane-1,2-diamine M-(3-phenoxybenzyl)ethane-1,2-diamine (86)

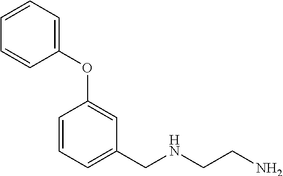 [SMILES: NCCNCc1cccc(Oc2ccccc2)c1]

To a solution of ethane-1,2-diamine (120 mg, 2 mmol)(150 mg, 2 mmol) in MeOH (5 mL) at 0° C. was added 3-(phenoxy)benzaldehyde (100 mg, 0.5 mmol) in small portions. After the addition, sodium borohydride (19 mg, 0.5 mmol) was added slowly in small portions at 0° C. The reaction was allowed to warm to room temperature overnight with stirring. The reaction was quenched with little water and concentrated to dryness. The crude was purified by reverse phase flash (MeCN/H₂O) to afford N₁-(3-phenoxyphenethyl)ethane-1,2-diamine (100 mg, 78% yield) as a yellow oil. MS Calc.: 256.2; MS Found: 257.3 [M+H]⁺.

### Preparation of 2,2-difluoro-N₁-(2-(naphthalen-2-yl)ethyl)propane-1,3-diamine 2,2-difluoromalonamide (87)

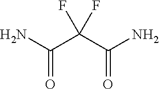

A mixture of diethyl 2,2-difluoromalonate (5.0 g, 25.5 mmol) in ammonia solution in methanol (100 mL) was stirred at room temperature for 16 hrs. The reaction mixture was concentrated to afford 2,2-difluoromalonamide (3.5g, yield: 99%) as a white solid.

### 2,2-difluoropropane-1,3-diamine (88)

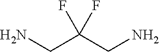

To a solution of 87 (3.5 g, 25.5 mmol) in THF (50 mL) was added BH3 in THF (12.7 mL, 127 mmol, 10N) at 0° C. The resulting mixture was refluxed overnight. After cooling to 0° C., the reaction was quenched with MeOH (20 mL) and concentrated. Then a solution of HCl in MeOH was added, and the mixture was stirred at room temperature for 30 min. The solid was filtered out and dried under vacuum to afford 2, 2-difluoropropane-1,3-diamine (2.1g, yield: 75%) as a colorless oil.

### 2-(naphthalen-2-yl)acetaldehyde (89)

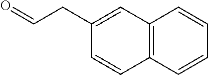 [SMILES: O=CCc1ccc2ccccc2c1]

To a solution of 2-(naphthalen-2-yl)ethan-1-ol (200 mg, 1.16 mmol) in DCM (10 mL) was added Dess-Martin periodinane (0.74 g, 1.75 mmol) and the solution was stirred at room temperature for 2 hrs. The reaction solution was diluted with DCM (20 mL) and washed with H₂O (10 mL), aq. Na₂SO₃ (10 ml), and aq. NaHCO₃ (10 ml). The organic phase was dried over Na₂SO₄ and concentrated to give 89 (70 mg, yield: 35%) as a brown oil.

### 2,2-difluoro-M-(2-(naphthalen-2-yl)ethyl)propane-1,3-diamine (90)

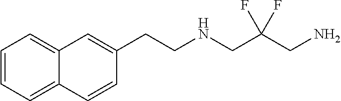 [SMILES: NCC(F)(F)CNCCc1ccc2ccccc2c1]

A solution of 89 (70 mg, 0.41 mmol) and 88 (68 mg, 0.62 mmol) in MeOH (3 mL) was stirred at room temperature for 20 min. NaBH₃CN (102 mg, 1.65 mmol) and AcOH (2 drops) was added, the solution was stirred at room temperature for 16 hrs. The resulting mixture was concentrated. The residue was purified via flash with the solvent of ACN in H₂O. The collected fraction was concentrated. This afforded the desired product 90 (50 mg, yield: 46%) as a white solid. MS Calc.: 264.1; MS Found: 265.0 [M+H⁺].

### Preparation of tert-butyl (3-aminopropyl)(4-phenoxyphenethyl)carbamate 2-(4-phenoxyphenyl)ethan-1-ol (91)

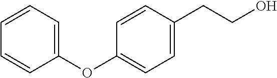 [SMILES: OCCc1ccc(Oc2ccccc2)cc1]

In a heat-dried three-necked round bottom flask, Phenylboronic acid (3.97 g, 32.57 mmol) and 2-(4-Hydroxyphenyl)ethanol (1.50 g, 10.86 mmol) were added in dry C₂H2Cl₂ (67.90 mL). Afterwards, dry pyridine (2.52 mL, 32.57 mmol), anhydrous copper acetate (2.96 g, 16.28 mmol), and 4 Å molecular sieves (1.87 g) were added to the mixture. The reaction mixture was stirred for 48 h at rt. The mixture was filtered off and the cake was rinsed several times with CH₂Cl₂. The filtrate was concentrated under vacuum to complete dryness. The obtained residue was purified over silica (cyclohexane/EtOAc; 10-80%) to afford the pure product (0.0.85 g, 37%). ¹H NMR (400 MHz, DMSO-d₆) δ7.43-7.33 (m, 2H, H3/5), 7.26-7.21 (m, 2H, H3′/5′), 7.15-7.09 (m, 1H, H4), 7.00-6.95 (m, 2H. H2/6), 6.95-6.91 (m, 2H, H2′/6′), 4.65 (t, J=5.2 Hz, 1H, OH), 3.60 (td, J=7.0, 5.2 Hz, 2H, CH₂, 1”), 2.71 (t, J=7.0 Hz, 2H, CH₂, 2″).

### 4-phenoxyphenethyl 4-methylbenzenesulfonate (92)

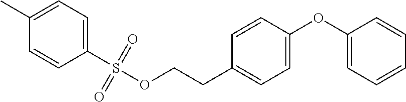 [SMILES: Cc1ccc(S(=O)(=O)OCCc2ccc(Oc3ccccc3)cc2)cc1]

The compound 91 (1.06 g, 4.97 mmol) was dissolved in dry CH₂Cl₂ (24.80 mL). Then, TsCl (0.95 g, 4.97 mmol) was added. Afterwards, dry pyridine (1.20 mL, 14.90 mmol) and DMAP (0.06 g, 0.50 mmol) were added 0° C. The solution was stirred at rt overnight. The mixture was diluted with saturated bicarbonate solution and extracted three times with CH₂Cl₂. The combined organic layers were washed with brine, dried over sodium sulfate, and concentrated under reduced pressure. The crude was purified by flash chromatography (cyclohexane/EtOAc; 0-80%) to afford the product as a colorless solid (1.06 g, 58%). ¹H NMR (400 MHZ, DMSO-d₆) δ7.74-7.65 (m, 2H, o-tosyl), 7.45-7.40 (m, 2H, m-tosyl), 7.40-7.36 (m, 2H, H3/5), 7.20-7.10 (m, 3H, H4/3′/5′), 7.02-6.94 (m, 2H, H2/6), 6.93-6.84 (m, 2H, H2′/6′), 4.22 (t, J=6.5 Hz, 2H, CH₂, 1′″), 2.87 (t, J=6.5 Hz, 2H, CH₂, 2″), 2.40 (s, 3H, CH₃)

### 3-((tert-butyldiphenylsilyl)oxy)-N-(4-phenoxyphenethyl)propan-1-amine (93)

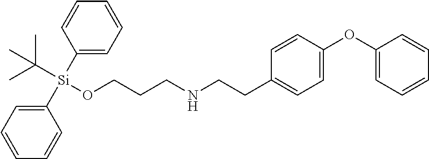 [SMILES: CC(C)(C)[Si](OCCCNCCc1ccc(Oc2ccccc2)cc1)(c1ccccc1)c1ccccc1]

A solution of 92 (0.63 g, 1.71 mmol) in dry DMF (4.25 mL) was added to a solution of 3-((tert-butyldiphenylsilyl)oxy)propan-1-amine (0.80 g, 2.57 mmol) in dry DMF (4.25 mL) at rt. Then, K₂CO₃ (0.47 g, 3.42 mmol) was added and the suspension was heated to 80° C. and stirred at this temperature overnight. TLC(cyclohexane/EtOAc; 50%) monitored full consumption of 92. Then, the reaction was allowed to cool down to rt and was diluted with saturated bicarbonate solution. The aqueous phase was extracted three times with EtOAc. Afterwards, the combined organic layers were washed with brine, dried over sodium sulfate, and concentrated under reduced pressure. The obtained residue was purified by flash chromatography (cyclohexane/EtOAc; 0-60%) to afford the pure product (0.34 g, 43%). ¹H NMR (400 MHZ, DMSO-d₆) δ7.66-7.58 (m, 4H, Ar—H), 7.49-7.40 (m, 6H, Ar—H), 7.40-7.34 (m, 2H, H3/5), 7.24-7.17 (m, 2H, H3′/5′), 7.16-7.07 (m, 1H, H4), 6.99-6.95 (m, 2H, H2/6), 6.92-6.89 (m, 2H, H2′/6′), 3.71 (t, J=6.3 Hz, 2H, CH₂, H1″), 2.72-2.60 (m, 6H, H3″/4″/5″), 1.67 (h, J=6.5 Hz, 2H, CH₂, H2″), 0.99 (s, 9H, CH₃, t-butyl).

### 3-((4-phenoxyphenethyl) amino)propan-1-ol(94)

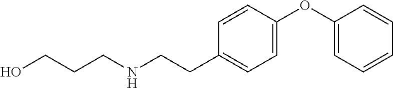 [SMILES: OCCCNCCc1ccc(Oc2ccccc2)cc1]

To a solution of 93 (0.33 g, 0.65 mmol) in dry THF (3.20 mL), TBAF (0.36 mL, 1.30 mmol) was added dropwise. Then, the solution was stirred magnetically at rt for 5 h. TLC(CH₂Cl₂/MeOH; 10%) indicated after this 5 h full conversion. The organic solvent was evaporated under vacuum and the obtained residue was purified over silica (CH₂Cl₂/MeOH; 0-20%) to afford the desired product (0.13 g, 71%). ¹H NMR (400 MHZ, DMSO-d₆) δ 7.42-7.34 (m, 2H, m″), 7.26-7.20 (m, 2H, m′), 7.17-7.07 (m, 1H, p″), 7.01-6.90 (m, 4H, o′, o″), 3.45 (t, J=6.3 Hz, 2H, CH₂, H1′), 2.78-2.66 (m, 4H, CH₂, H4′, H5′), 2.63 (t, J=6.9 Hz, 2H, CH₂, H3′), 1.56 (p, J=6.6 Hz, 2H, CH₂, H2′).

### tert-butyl (3-hydroxypropyl)(4-phenoxyphenethyl)carbamate (95)

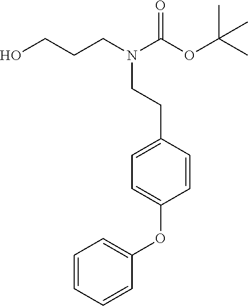 [SMILES: CC(C)(C)OC(=O)N(CCCO)CCc1ccc(Oc2ccccc2)cc1]

Triethylamine (0.12 mL, 0.83 mmol) was added to a solution of 94 (0.15 g, 0.55 mmol) in dry CH₂Cl₂ (2.80 mL). The solution was cooled down to 0° C. in an ice-bath. Afterwards, di-tert-butyl dicarbonate (0.14 mL, 0.61 mmol) was added portion wise. Then, the reaction mixture was allowed to warm up to rt and stirred for 5 h till complete conversion was monitored by TLC (cyclohexane/EtOAc; 80%). The mixture was diluted with saturated sodium bicarbonate solution and the organic phase was separated. The aqueous phase was extracted three times with CH₂Cl₂. The combined organic layers were washed with brine, dried over sodium sulfate, and concentrated over vacuum. The crude was purified by flash chromatography (cyclohexane/EtOAc; 0-80%) to afford a colorless oil (0.18 g, 88%). ¹H NMR (400 MHZ, DMSO-d₆) δ 7.42-7.34 (m, 2H, m″), 7.21 (d, J=8.0 Hz, 2H, m′), 7.15-7.09 (m, 1H, p″), 7.01-6.91 (m, 4H, o′, o″), 4.42 (t, J=5.2 Hz, 1H, OH), 3.42-3.30 (m, 4H, CH₂, H1, H4′), 3.21-3.11 (m, 2H, CH₂, H3′), 2.75 (t, J=7.4 Hz, 2H, CH₂, H5′), 1.60 (p, J=6.5 Hz, 2H, CH₂, H2′), 1.36 (s, 9H, CH₃, &Bu).

### tert-butyl (3-azidopropyl)(4-phenoxyphenethyl)carbamate (96)

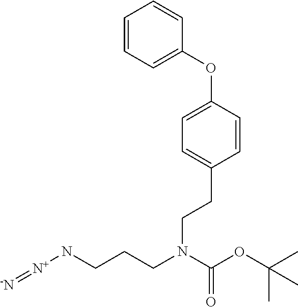

Compound 95 (0.09 g, 0.24 mmol) was dissolved in dry 1,4-dioxane (1.20 mL) and cooled down to 0° C. To the cooled solution DPPA (0.10 mL, 0.48 mmol) was added followed by DBU (0.11 mL, 0.72 mmol). The reaction mixture was stirred for 17 h at rt. Then, NaN₃ (0.08 g, 1.2 mmol) and 15-crown-5 (0.05 mL, 0.24 mmol) were added to the suspension and heated to 110° C. After 6 h, the organic solvent was removed under vacuum and then water was added. The aqueous phase was extracted with EtOAc. The combined organic layers were washed with brine, dried over sodium sulfate, and concentrated. ¹H NMR (400 MHZ, DMSO-d₆) δ 7.41-7.34 (m, 2H, m″), 7.22 (d, J=8.0 Hz, 2H, m′), 7.15-7.09 (m, 1H, p′), 7.01-6.92 (m, 4H, o′, o″), 3.38-3.27 (m, 4H, CH₂, H3′, H4′), 3.22-3.14 (m, 2H, CH₂, H1′), 2.75 (t, J=7.4 Hz, 2H, CH₂, H5′), 1.70 (p, J=6.8 Hz, 2H, CH₂, H2′), 1.44-1.31 (m, 9H CH₃, Bu).

### tert-butyl (3-aminopropyl)(4-phenoxyphenethyl)carbamate (97)

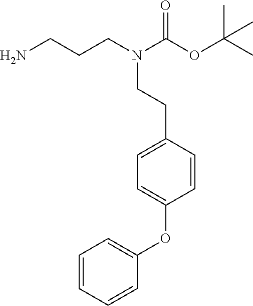 [SMILES: CC(C)(C)OC(=O)N(CCCN)CCc1ccc(Oc2ccccc2)cc1]

To a solution of 96 (0.07 g, 0.18 mmol) in EtOH (1.70 mL) palladium on activated charcoal moistened with water (0.04 g, 0.02 mmol) was added under nitrogen atmosphere at rt. Then, the reaction mixture was purged with hydrogen and stirred for 2 h at rt. The reaction progress was monitored by TLC(cyclohexane/EtOAc; 20%). Then, the reaction mixture was purged with nitrogen and the catalyst was filtered off. The filtrate was concentrated under reduced pressure and the obtained crude product was purified over silica (CH₂Cl₂/MeOH; 0-20%) to afford the product as colorless oil (0.05 g, 69%). ¹H NMR (400 MHZ, DMSO-d₆) δ 7.42-7.34 (m, 2H, m″), 7.25-7.18 (m, 2H, m′), 7.15-7.09 (m, 1H, p″), 7.00-6.91 (m, 4H, o′, o″), 3.39-3.28 (m, 2H, CH₂, H4′), 3.22-3.12 (m, 2H, CH₂, H3′), 2.75 (t, J=7.4 Hz, 2H, CH₂, H5′), 2.54-2.52 (m, 2H, H1′), 1.53 (p, J=6.9 Hz, 2H, CH₂, H2′), 1.41-1.32 (m, 9H CH₃, &Bu).

### Preparation of tert-butyl (3-aminopropyl)(3-phenoxyphenethyl)carbamate 2-(3-phenoxyphenyl)ethan-1-ol (98)

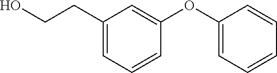 [SMILES: OCCc1cccc(Oc2ccccc2)c1]

In a heat-dried three-necked round bottom flask, Phenylboronic acid (3.97 g, 32.57 mmol) and 2-(3-Hydroxyphenyl)ethanol (1.50 g, 10.86 mmol) were added in dry C₂H2Cl₂ (67.90 mL).

Afterwards, dry pyridine (2.52 mL, 32.57 mmol), anhydrous copper acetate (2.96 g, 16.28 mmol), and 4 Å molecular sieves (1.87 g) were added to the mixture. The reaction mixture was stirred for 48 h at rt. The mixture was filtered off and the cake was rinsed several times with CH₂Cl₂. The filtrate was concentrated under vacuum to complete dryness. The obtained residue was purified over silica (cyclohexane/EtOAc; 10-80%) to afford the pure product (0.58 g, 25%) as a yellowish oil. ¹H NMR (400 MHZ, DMSO-d₆) δ7.42-7.36 (m, 2H, H3/5), 7.33-7.23 (m, 1H, H5′), 7.18-7.09 (m, 1H, H4), 7.05-6.96 (m, 3H, H2/6/6′), 6.90-6.88 (m, 1H, H2′), 6.81 (ddd, J=8.1, 2.5, 1.0 Hz, 1H, H4′), 4.64 (t, J=5.2 Hz, 1H, OH), 3.60 (td, J=7.0, 5.2 Hz, 2H, CH₂, 1″), 2.71 (t, J=6.9 Hz, 2H, CH₂, H2″).

### 3-phenoxyphenethyl 4-methylbenzenesulfonate (99)

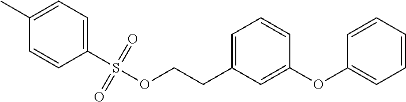 [SMILES: Cc1ccc(S(=O)(=O)OCCc2cccc(Oc3ccccc3)c2)cc1]

The compound 98 (0.55 g, 2.57 mmol) was dissolved in dry CH₂Cl₂ (12.80 mL). Then, TsCl (0.49 g, 2.57 mmol) was added. Afterwards, dry pyridine (0.62 mL, 7.70 mmol) and DMAP (0.03 g, 0.26 mmol) were added at 0° C. The solution was stirred at rt overnight. TLC (cyclochexane/EtOAc, 20%) monitored no complete conversion. At this point further dry pyridine (0.31 mL, 3.85 mmol) was added and stirred magnetically for 8 h at rt. Then, the mixture was diluted with saturated bicarbonate solution and extracted three times with CH₂Cl₂. The combined organic layers were washed with brine, dried over sodium sulfate, and concentrated under reduced pressure. The crude was purified by flash chromatography (cyclohexane/EtOAc; 0-60%) to afford the product as a colorless oil (0.66 g, 70%). ¹H NMR (400 MHZ, DMSO-d₆) δ7.69-7.65 (m, 2H, o-tosyl), 7.45-7.40 (m, 2H, m-tosyl), 7.40-7.36 (m, 2H, H3/5), 7.32-7.23 (m, 1H, H5′), 7.19-7.10 (m, 1H, H4), 6.99-6.96 (m, 2H, H2/6), 6.98-6.91 (m, 1H, H4′), 6.87-6.81 (m, 2H, H2′/6′), 4.23 (t, J=6.3 Hz, 2H, CH₂, 1″), 2.88 (t, J=6.3 Hz, 2H, CH₂, H2″), 2.41 (s, 3H, CH₃)

### 3-((tert-butyldiphenylsilyl)oxy)-N-(3-phenoxyphenethyl)propan-1-amine (100)

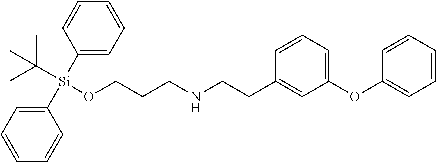 [SMILES: CC(C)(C)[Si](OCCCNCCc1cccc(Oc2ccccc2)c1)(c1ccccc1)c1ccccc1]

A solution of 99 (0.62 g, 1.69 mmol) in dry DMF (4.20 mL) was added to a solution of 3-((tert-butyldiphenylsilyl)oxy)propan-1-amine (0.79 g, 2.53 mmol) in dry DMF (4.20 mL) at rt. Then, K₂CO₃ (0.47 g, 3.38 mmol) was added and the suspension was heated to 80° C. and stirred at this temperature overnight. TLC(cyclohexane/EtOAc; 50%) monitored full consumption of 99. Then, the reaction was allowed to cool down to rt and was diluted with saturated bicarbonate solution. The aqueous phase was extracted three times with EtOAc. Afterwards, the combined organic layers were washed with brine, dried over sodium sulfate, and concentrated under reduced pressure. The obtained residue was purified by flash chromatography (cyclohexane/EtOAc; 0-60%) to afford the pure product (0.29 g, 34%). ¹H NMR (400 MHZ, DMSO-d₆) δ7.66-7.57 (m, 4H, Ar—H), 7.49-7.38 (m, 6H, Ar—H), 7.42-7.33 (m, 2H, H3/5), 7.32-7.23 (m, 1H, H5′), 7.17-7.08 (m, 1H. H4), 7.04-6.94 (m, 3H, H2/4′/6), 6.88-6.77 (m, 2H, H2′/6′), 3.70 (t, J=6.3 Hz, 2H, CH₂, 1″), 2.73-2.60 (m, 4H, CH₂, 4″/5′), 2.60 (t, J=6.8 Hz, 2H, CH₂, 3″), 1.64 (p, J=6.5 Hz, 2H, CH₂, H2″), 0.99 (s, 9H, CH₃, t-butyl).

### 3-((3-phenoxyphenethyl) amino)propan-1-ol(101)

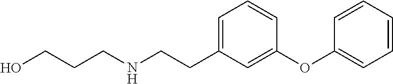 [SMILES: OCCCNCCc1cccc(Oc2ccccc2)c1]

To a solution of 100 (0.30 g, 0.59 mmol) in dry THF (2.90 mL), TBAF (0.32 mL, 1.18 mmol) was added dropwise. Then, the solution was stirred magnetically at rt for 5 h. TLC(CH₂Cl₂/MeOH; 10%) indicated after this 5 h full conversion. The organic solvent was evaporated under vacuum and the obtained residue was purified over silica (CH₂Cl₂/MeOH; 0-20%) to afford the desired product (0.13 g, 75%). ¹H NMR (400 MHZ, DMSO-d₆) δ7.41-7.35 (m, 2H, H3/H5), 7.28 (t, J=7.8 Hz, 1H, H5′), 7.17-7.08 (m, 1H, H4), 7.02-6.97 (m, 3H, H2/6/4′), 6.88-6.85 (m, 1H, H2′), 6.81 (ddd, J=8.1, 2.5, 0.9 Hz, 1H, H6′), 3.42 (t, J=6.3 Hz, 2H, CH₂, 1″), 2.76-2.64 (m, 4H, CH₂, H4″/H5″), 2.58 (t, J=6.9 Hz, 2H, CH₂, H3″), 1.53 (p, J=6.6 Hz, 2H, CH₂, H2″).

### tert-butyl (3-hydroxypropyl)(3-phenoxyphenethyl)carbamate (102)

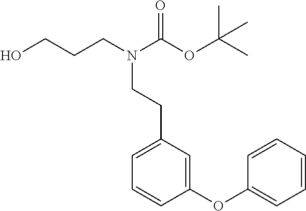 [SMILES: CC(C)(C)OC(=O)N(CCCO)CCc1cccc(Oc2ccccc2)c1]

Triethylamine (0.11 mL, 0.77 mmol) was added to a solution of 101 (0.14 g, 0.52 mmol) in dry CH₂Cl₂ (2.60 mL). The solution was cooled down to 0° C. in an ice-bath. Afterwards, di-tert-butyl dicarbonate (0.13 mL, 0.57 mmol) was added portion wise. Then, the reaction mixture was allowed to warm up to rt and stirred for 5 h till complete conversion was monitored by TLC (cyclohexane/EtOAc; 50%). The mixture was diluted with saturated sodium bicarbonate solution and the organic phase was separated. The aqueous phase was extracted three times with CH₂Cl₂. The combined organic layers were washed with brine, dried over sodium sulfate, and concentrated over vacuum. The crude was purified by flash chromatography (cyclohexane/EtOAc; 0-80%) to afford a colorless oil (0.15 g, 73%). ¹H NMR (400 MHZ, DMSO-d₆) δ7.42-7.36 (m, 2H, H3,5), 7.31 (t, J=7.9 Hz, 1H, H5’), 7.14 (t, J=7.3 Hz, 1H, H4), 7.03-6.94 (m, 3H, H3,5,4′), 6.85 (d, J=7.5 Hz, 2H, H2′,6′), 4.42 (t, J=4.8 Hz, 1H, OH), 3.40-3.29 (m, 4H, CH₂, H1″,4″), 3.19-3.05 (m, 2H, CH₂, H3″), 2.74 (t, J=7.3 Hz, 2H, CH₂, H5″), 1.58 (p, J=6.6 Hz, 2H, CH₂, H2″), 1.38-1.28 (m, 9H, CH₃, t-butyl).

### tert-butyl (3-azidopropyl)(3-phenoxyphenethyl)carbamate (103)

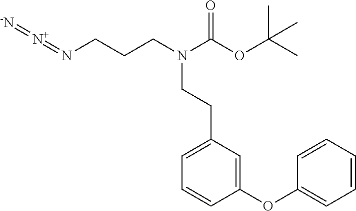

To a stirred solution of 102 (0.14 g, 0.37 mmol) in CH₂Cl₂ (1.90 mL) Et₃N (0.08 mL; 0.56 mmol) and MsCl (0.04 mL, 0.45 mmol) were added at 0° C. under nitrogen atmosphere. After 1 h stirring at rt, the reaction was diluted with brine. The organic layer was separated, and the aqueous phase was extracted with CH₂Cl₂. The combined organic layers were dried over sodium sulfate and concentrated over vacuum to complete dryness. The mesylated product was dissolved in dry DMF (1.90 mL). To this solution NaN₃ (0.07 g, 1.12 mmol) was added and heated to 60° C. After 5 h, TLC(cyclohexane/EtOAc; 50%) monitored full consumption. Then, the organic solvent was removed under reduced pressure. Water was added and the aqueous phase was extracted with EtOAc. The combined organic layers were washed with brine, dried over sodium sulfate, and concentrated over vacuum. The obtained residue was purified over silica (cyclohexane/EtOAc; 0-30%). ¹H NMR (400 MHZ, DMSO-d₆) δ7.42-7.36 (m, 2H, H3/5), 7.31 (dd, J=8.4, 7.6 Hz, 1H, H5′), 7.18-7.10 (m, 1H, H4), 7.03-6.95 (m, 3H, H2,6,4′), 6.88-6.83 (m, 2H, H2′,6′), 3.37-3.27 (m, 4H, CH₂, H1″,4″), 3.21-3.06 (m, 2H, CH₂, H3″), 2.75 (t, J=7.2 Hz, 2H, CH₂, H5″), 1.67 (p, J=6.7 Hz, 2H, CH₂, H2″), 1.43-1.27 (m, 9H, CH₃, t-butyl).

### tert-butyl (3-aminopropyl)(3-phenoxyphenethyl)carbamate (104)

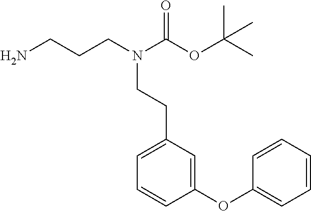 [SMILES: CC(C)(C)OC(=O)N(CCCN)CCc1cccc(Oc2ccccc2)c1]

Compound 103 (0.09 g, 0.23 mmol) was dissolved in EtOH (2.30 mL). This solution was degassed and stirred at rt under nitrogen. Afterwards, palladium on activated charcoal moistened with water (0.05 g, 0.02 mmol) was added and the resulted suspension was degassed purged with nitrogen. Then, the mixture was set under hydrogen atmosphere with a storage vessel and stirred at rt for 2 h. TLC(cyclohexane/EtOAc; 20%) showed full consumption of 103. Thus, the mixture was purged with nitrogen and filtered off. The filter cake was rinsed with EtOH and CH₂Cl₂. The filtrate was concentrated under reduced pressure. The crude product was purified by flash chromatography (CH₂Cl₂/MeOH; 0-20%) to afford the product as colorless oil (0.05 g, 59%). ¹H NMR (400 MHZ, DMSO-d₆) δ7.42-7.36 (m, 2H, H3,5), 7.34-7.28 (m, 1H, H5′), 7.14 (t, J=7.4 Hz, 1H, H4), 7.03-6.95 (m, 3H, H2,6,4′), 6.90-6.80 (m, 2H, H2′,6′), 3.35-3.28 (m, 2H, CH₂, H4″), 3.19-3.05 (m, 2H, CH₂, H1″), 2.78-2.71 (m, 2H, CH₂, H5″), 2.49-2.45 (m, 2H, CH₂, H3″), 1.50 (p, J=6.9 Hz, 2H, CH₂, H2″), 1.41-1.26 (m, 9H, CH₃, t-butyl).

### Preparation of tert-butyl (3-aminopropyl)(phenethyl)carbamate 3-(phenethylamino)propan-1-ol(105)

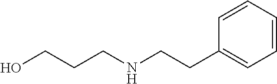 [SMILES: OCCCNCCc1ccccc1]

To a solution of 2-phenylacetaldehyde (0.60 g, 5.00 mmol) in dry MeOH (10 mL) was added 3-aminopropan-1-ol (0.38 g, 5.00 mmol) dropwise at rt. The reaction mixture was stirred overnight at rt. TLC monitored complete conversion. The reaction was then cooled down in an ice-bath. Then, NaBH₄ (0.28 g, 7.50 mmol) was added portion wise to the solution. After the bubbling hat stopped, the solvent was evaporated under vacuum. The obtained residue was diluted with water and extracted with EtOAc. The organic layer was washed with brine, dried over sodium sulfate, and concentrated under reduced pressure. The obtained crude was purified by flash chromatography (EtOAc 100%, then DCM/MeOH; 0-20%) to afford the product (0.82 g, 92%). 1H NMR (400 MHZ, DMSO-d₆) δ7.31-7.25 (m, 1H), 7.23-7.15 (m, 2H), 3.44 (t, J=6.3 Hz, 1H), 2.70 (dt, J=4.4, 2.4 Hz, 3H), 2.58 (t,)=6.8 Hz, 1H), 1.54 (p, J=6.6 Hz, 1H).

### tert-butyl (3-hydroxypropyl)(phenethyl)carbamate (106)

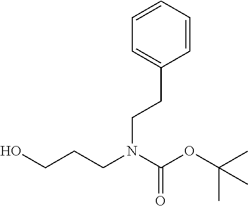 [SMILES: CC(C)(C)OC(=O)N(CCCO)CCc1ccccc1]

Compound 105 (0.46 g, 2.57 mmol) in CH₂Cl₂ (12.80 mL) Et₃N (0.53 mL, 3.85 mmol) was added. Then, the solution was cooled down in an ice-bath. Afterwards, boc anhydride (0.65 mL, 2.82 mmol) was added portionwise to the cooled solution. The reaction mixture was allowed to warm up to rt and stirred magnetically at this temp until complete conversion. TLC was used to monitor the progress (cyclohexane/EtOAc; 50%) & (DCM/MeOH; 20%). After 3 h, the solution was diltued with sat. bicarbonate solution and extracted with EtOAc. The combined organic layers were washed with brine, dried over sodium sulfate, and concentrated under reduced pressure. The obtained residue was purified over silica (cyclohexane/EtOAc; 20-100%) to afford the product (0.60 g, 83%). ¹H NMR (400 MHZ, DMSO-d₆) δ 7.33-7.26 (m, 2H, H3, H5), 7.24-7.16 (m, 3H, H2, H4, H6), 4.43 (t, J=5.0 Hz, 1H, OH), 3.41-3.30 (m, 4H, CH₂, H4′, H5′), 3.21-3.09 (m, 2H, CH₂, H1′), 2.75 (t, 2H, CH₂, H3′), 1.60 (p, J=6.4 Hz, 2H, CH₂, H2′), 1.46-1.27 (m, 9H, CH₃, t-butyl).

### tert-butyl (3-azidopropyl)(phenethyl)carbamate (107)

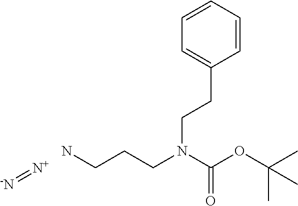

To a stirred solution of 106 (0.10 g, 0.35 mmol) in CH₂Cl₂ (1.80 mL) Et₃N (0.07 mL; 0.53 mmol) and MsCl (0.03 mL, 0.05 mmol) were added at 0° C. under nitrogen atmosphere. After 1 h stirring at rt, the reaction was diluted with brine. The organic layer was separated, and the aqueous phase was extracted with CH₂Cl₂. The combined organic layers were dried over sodium sulfate and concentrated over vacuum to complete dryness. The mesylated product was dissolved in dry DMF (1.80 mL). To this solution NaN₃ (0.07 g, 1.06 mmol) was added and heated to 60° C. After 5 h, TLC(cyclohexane/EtOAc; 50%) monitored full consumption. Then, the organic solvent was removed under reduced pressure. Water was added and the aqueous phase was extracted with EtOAc. The combined organic layers were washed with brine, dried over sodium sulfate, and concentrated over vacuum. The obtained residue was purified over silica (cyclohexane/EtOAc; 0-30%) to afford a colorless oil. ¹H NMR (400 MHZ, DMSO-d₆) δ 7.36-7.26 (m, 2H, o′), 7.25-7.17 (m, 3H, m′, p′), 3.34-3.27 (m, 2H, CH₂, H3′, H4′), 3.22-3.11 (m, 2H, CH₂, H1′), 2.81-2.70 (m, 2H, CH₂, H5′), 1.69 (p, J=6.7z Hz, 2H, CH₂, H2′), 1.43-1.29 (m, 9H, CH₃, (Bu).

### tert-butyl (3-aminopropyl)(phenethyl)carbamate (108)

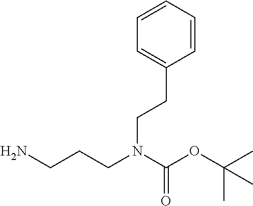 [SMILES: CC(C)(C)OC(=O)N(CCCN)CCc1ccccc1]

To a solution of 107 (0.11 g, 0.36 mmol) in EtOH (3.60 mL) palladium on activated charcoal moistened with water (0.08 g, 0.04 mmol) was added under nitrogen atmosphere at rt. Then, the reaction mixture was purged with hydrogen and stirred for 2 h at rt. The reaction progress was monitored by TLC(cyclohexane/EtOAc; 20%). Then, the reaction mixture was purged with nitrogen and the catalyst was filtered off. The filtrate was concentrated under reduced pressure and the obtained crude product was purified over silica (CH₂Cl₂/MeOH; 0-10%) to afford the product as colorless oil (0.05 g, 69%). ¹H NMR (400 MHZ, DMSO-d₆) δ 7.34-7.25 (m, 2H, o′), 7.25-7.17 (m, 3H, m′, p′), 3.43-3.26 (m, 2H, CH₂, H4′), 3.21-3.09 (m, 2H, CH₂, H3′), 2.81-2.71 (m, 2H, CH₂, H5′), 1.60-1.47 (m, 2H, CH₂, H2′), 1.42-1.28 (m, 9H, CH₃, (Bu).

### Preparation of N-(3-aminopropyl)-2-phenylacetamide

### N-(3-aminopropyl)-2-phenylacetamide (109)

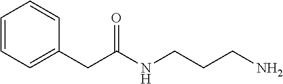 [SMILES: NCCCNC(=O)Cc1ccccc1]

A solution of phenylacetylchloride (2.00 g, 14.48 mmol) in dry CH₂Cl2 was added over 2 h to a solution of 3-propanmediamine (12.18 mL, 144.75 mmol) and triethylamine (4.05 mL, 28.95 mmol) in dry CH₂Cl₂ at ambient temperature. The mixture was stirred for 24 h at rt. The organic solvent was removed under vacuum and the residue was taken up with CH₂Cl₂ and brine. The organic layer was separated, and the aqueous phase was extracted with CH₂Cl₂. The combined organic layers were dried over sodium sulfate, and concentrated. The obtained residue was purified over NH-coated silica (DCM/MeOH; 0-10%) to afford the compound as a colorless oil (0.20 g, 7%). NMR according to literature data.

### Preparation of N-(3-aminopropyl)propionamide

### tert-butyl (3-propionamidopropyl)carbamate (110)

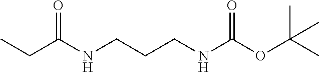 [SMILES: CCC(=O)NCCCNC(=O)OC(C)(C)C]

A solution of propionyl chloride (0.50 g, 5.40 mmol) in dry CH₂Cl₂ was added to a solution of tert-butyl (3-aminopropyl)carbamate (1.14 mL, 6.49 mmol) and triethylamine (1.13 mL, 8.11 mmol) in dry CH₂Cl₂ at ambient temperature. The mixture was stirred for 24 h at rt. The organic solvent was removed under vacuum and the residue was taken up with CH₂Cl₂ and brine. The organic layer was separated, and the aqueous phase was extracted with CH₂Cl₂. The combined organic layers were dried over sodium sulfate, and concentrated. The crude product was purified over silica ( ) to afford the desired product was a colorless oil (0.42 g, 34%). ¹H NMR (400 MHZ, DMSO-d₆) δ7.72 (t, J=5.0 Hz, 1H, NH), 6.78 (t, J=5.5 Hz, 1H, NH), 3.01 (q, J=6.8 Hz, 2H, CH₂, H3), 2.91 (q, J=6.7 Hz, 2H, CH₂, H·), 2.05 (q, J=7.6 Hz, 2H, CH₂, H2′), 1.48 (p, J=6.9 Hz, 2H, CH₂, H2), 1.38 (s, 9H, CH₃. t-butyl), 0.98 (t, J=7.6 Hz, 3H, CH₃, HB).

### N-(3-aminopropyl)propionamide (111)

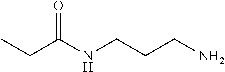

Compound 110 (0.37 g, 1.59 mmol) was dissolved in CH₂Cl₂ (6.40 mL). Then, TFA (1.60 mL) was added, and the solution was stirred at ambient temperature for 3 h. TLC(CH₂Cl₂/MeOH; 10%) monitored full conversion. Then, the solution was concentrated to complete dryness which afforded the product as colorless oil (0.39 g, quant.; 1 TFA salt). ¹H NMR (400 MHZ, DMSO-d₆) δ 7.94 (t, J=5.4 Hz, 1H, NH), 7.72 (bs, 3H, NH₃+), 3.10 (q, J=6.7 Hz, 2H, CH₂, H3), 2.83-2.72 (m, 2H, CH₂, H1), 2.09 (q, J=7.6 Hz, 2H, CH₂, H·), 1.66 (p, J=14.2, 6.8 Hz, 2H, CH₂, H2), 1.00 (t, J=7.6 Hz, 3H, CH₃, HB).

### Preparation of 1-(3-aminopropyl)-3-phenylurea

### tert-butyl (3-(3-phenylureido)propyl)carbamate (106h)

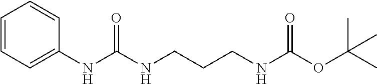 [SMILES: CC(C)(C)OC(=O)NCCCNC(=O)Nc1ccccc1]

To a cooled solution of tert-butyl 3-aminopropylcarbamate (0.74 g, 4.20 mmol) in dry CH₂Cl₂ (21.0 mL)phenylisocyanate (0.50 g, 4.20 mmol) was added and stirred overnight at rt. Then, the solvents were removed by rotatory evaporation. The obtained residue was purified over silica (cyclohexane/EtOAc; 20-80%) to afford the product as white solid (79%). ¹H NMR (400 MHZ, DMSO-d₆) δ8.49 (s, 1H, NH), 7.38 (dt, J=8.8, 1.6 Hz, 2H, H3, H5), 7.25-7.18 (m, 2H, H2, H6), 6.93-6.83 (m, 1H, H4), 6.84 (t, J=5.9 Hz, 1H, NH), 6.13 (t, J=5.7 Hz, 1H, NH), 3.07 (q, J=6.6 Hz, 2H, CH₂, H3), 2.96 (q, J=6.6 Hz, 2H, CH₂, H1), 1.51 (p, J=6.7, 6.1 Hz, 2H, CH₂, H2), 1.39 (s, 9H, CH₃, t-butyl).

### 1-(3-aminopropyl)-3-phenylurea (107h)

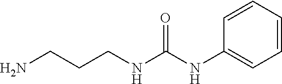 [SMILES: NCCCNC(=O)Nc1ccccc1]

Compound 106h (0.91 g, 3.11 mmol) was dissolved in CH₂Cl₂ (12.40 mL). Then, TFA (3.10 mL) was added and the mixture was stirred at rt for 3 h. TLC(CH₂Cl₂ MeOH; 10%) showed complete conversion. The reaction mixture was concentrated to complete dryness over vacuum which afforded the desired compound as colorless solid (0.61 g, quant.; 1 TFA salt). ¹H NMR (400 MHZ, DMSO-d₆) δ8.69 (s, 1H, NH), 7.72 (s, 3H, NH₃+), 7.42-7.37 (m, 2H, H3, H5), 7.25-7.19 (m, 2H, H2, H6), 6.90 (tt, J=7.5, 1.2 Hz, 1H, H4), 6.47 (s, 1H, NH), 3.16 (t, J=6.3 Hz, 2H, CH₂, H3), 2.89-2.76 (m, 2H, CH₂, H1), 1.71 (p, J=6.7 Hz, 2H, CH₂, H2)

### Preparation of 1-(3-aminopropyl)-3-ethylurea tert-butyl (3-(3-ethylureido)propyl)carbamate (108h)

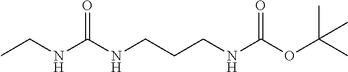 [SMILES: CCNC(=O)NCCCNC(=O)OC(C)(C)C]

To a cooled solution of tert-butyl 3-aminopropylcarbamate (1.24 g, 7.03 mmol) in dry CH₂Cl₂ (35.20 mL) ethylisocyanate (0.50 g, 7.03 mmol) was added and stirred overnight at rt. Then, the solvents were removed by rotatory evaporation. The obtained residue was purified over silica (cyclohexane/EtOAc; 20-80%) to afford the product as white solid (34%). ¹H NMR (400 MHZ, DMSO-d₆) δ6.79 (t, J=5.7 Hz, 1H, NH), 5.84-5.74 (m, 2H, NH), 3.03-2.93 (m, 4H, CH₂, H1′, H3), 2.90 (q, J=6.8 Hz, 2H, CH₂, H1), 1.48-1.41 (m, 2H, CH₂, H2), 1.38 (s, 9H, CH₃, t-butyl), 0.97 (t, J=7.2 Hz, 3H, CH₃, H2′).

### 1-(3-aminopropyl)-3-ethylurea (109h)

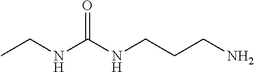

To a solution of compound 108h (1.05 g, 4.30 mmol) in CH₂Cl₂ (17.20 mL) was added TFA (4.30 mL) and stirred magnetically at rt for 3 h. After this time the solvents were removed under vacuum which afforded the compound as a colorless oil (1.20 g, quant.; 1 TFA salt). ¹H NMR (400 MHZ, DMSO-d₆) δ7.74 (s, 3H, NH₃+), 3.06 (t, J=6.5 Hz, 2H, CH₂, H3), 3.01 (q, J=7.2 Hz, 2H, CH₂, H1′), 2.76 (h, J=5.9 Hz, 2H, CH₂, H1), 1.63 (p, J=6.8 Hz, 2H, CH₂, H2), 0.99 (t, J=7.2 Hz, 3H, CH₃, H2′).

### Nucleosides building blocks

### Preparation of tert-butyl (3-aminopropyl)(((3αR,4R,6R,6αR)-6-(6-((tert-butoxycarbonyl) amino)-9H-purin-9-yl)-2,2-dimethyltetrahydrofuro[3,4-d][1,3]dioxol-4-yl)methyl)carbamate tert-butyl (9-((3αR,4R,6R,6αR)-6-(((3-((((9H-fluoren-9-yl) meth-oxy)carbonyl) amino)propyl) amino)methyl)-2,2-dimethyltetrahydrofuro[3,4-d][1,3]dioxol-4-yl)-9H-purin-6-yl)carbamate (112)

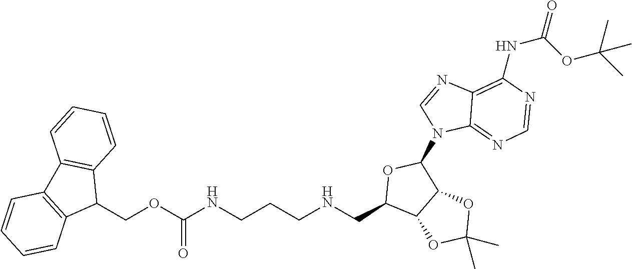 [SMILES: CC(C)(C)OC(=O)Nc1ncnc2c1ncn2[C@@H]1O[C@H](CNCCCNC(=O)OCC2c3ccccc3-c3ccccc32)[C@H]2OC(C)(C)O[C@H]21]

To a solution of aldehyde 51 (0.71 g, 2.37 mmol) in dry DCE (10.75 mL) was added drop wise as solution of 138 (0.88 g, 2.15 mmol) in dry DCE (10.75 mL). The solution was stirred 2 h at ambient temperature and then STAB (0.69 g, 3.23 mmol) was added portion wise over 30 min at ambient temperature. The mixture was stirred for 48 h and then quenched with an aqueous 1 m K₂CO₃ solution to reach pH ˜8. The organic layer was separated, and the aqueous phase was extracted with CH₂Cl₂. The combined organic layers were washed with brine, dried over sodium sulfate, and concentrated over vacuum. The obtained residue was purified over silica (CH₂Cl₂/MeOH; 0-10%) to afford the product as white foam (0.60 g, 41%). ¹H NMR (400 MHZ, DMSO-d₆) δ8.66 (s, 1H, H2, adenine), 8.63 (s, 1H, H8, adenine), 7.90 (dd, J=0.8 Hz, 1H, H5, Fmoc), 7.88 (dd, J=1.2, 0.7 Hz, 1H, H4, Fmoc), 7.86 (dd, J=0.8 Hz, 1H, H8, Fmoc), 7.87-7.81 (m, 1H, H1, Fmoc), 7.42 (td, J=7.4, 1.2 Hz, 2H, H2/7, Fmoc), 7.35 (td, J=7.4, 1.2 Hz, 2H, H3/6, Fmoc), 6.29 (s, 2H, CH₂, Fmoc), 6.19 (d, J z=2.6 Hz, 1H, H1′), 5.49 (dd, J=6.1, 2.6 Hz, 1H, H2′), 4.99 (dd, J=6.1, 2.3 Hz, 1H, H3′), 4.28-4.20 (m, 1H, H4′), 2.97-2.79 (m, 2H, CH₂, H3″), 2.74-2.58 (m, 2H, CH₂, H5′),, 2.49-2.43 (m, 2H, CH₂, H1″), 1.55 (s, 3H, CH₃, acetonide), 1.48 (s, 9H, CH₃, t-butyl), 1.48-1.45 (m, 2H, CH₂, H2″), 1.33 (s, 3H, CH₃, acetonide).

### tert-butyl (3-((((9H-fluoren-9-yl)methoxy)carbonyl)amino)propyl)(((3αR,4R,6R,6αR)-6-(6-((tert-butoxycarbonyl)amino)-9H-purin-9-yl)-2,2-dimethyltetrahydrofuro[3,4-d][1,3]dioxol-4-yl)methyl)carbamate (113)

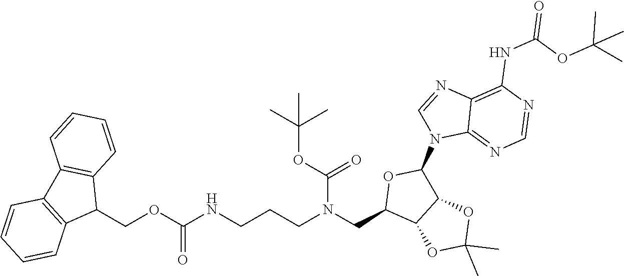 [SMILES: CC(C)(C)OC(=O)Nc1ncnc2c1ncn2[C@@H]1O[C@H](CN(CCCNC(=O)OCC2c3ccccc3-c3ccccc32)C(=O)OC(C)(C)C)[C@H]2OC(C)(C)O[C@H]21]

To cooled a solution of compound 112 (0.59 g, 0.85 mmol) in dry CH₂Cl₂ (8.50 mL), di-tert-butyl decarbonate (0.22 mL, 0.94 mmol) was added. The reaction solution was stirred overnight at ambient temperature. After 17 h, reaction mixture was diluted with sodium bicarbonate solution. The organic layer was separated, and the aqueous phase was extracted with CH₂Cl₂. The combined organic layers were washed with brine, dried over sodium sulfate, and concentrated. The obtained residue was purified by flash chromatography (CH₂Cl₂/MeOH; 0-7%) to afford the product as white foam (0.55 g, 82%). ¹H NMR (400 MHZ, DMSO-d₆) δ10.18 (s, 1H, NHBoc), 8.64 (s, 1H, H2, adenine), 8.63 (s, 1H, H8, adenine), 7.89 (d, J=7.4 Hz, 2H, H4/5, Fmoc), 7.67 (d, J=7.2 Hz, 2H, H1/8, Fmoc), 7.41 (t, J=7.4 Hz, 2H, H3/6, Fmoc), 7.32 (t, J=7.4 Hz, 2H, H2/7, Fmoc), 7.19 (t, J=5.3 Hz, 1H, NH), 6.28 (s, 1H, H1′), 5.56-5.47 (m, 1H, H2′), 5.08-4.98 (m, 1H, H3′), 4.32 (dd, J=6.5, 2.3 Hz, 1H, H4′), 4.32-4.22 (m, 2H, CH₂, Fmoc), 3.58-3.47 (m, 1H, CH₂, H5′), 3.32-3.19 (m, 1H, CH₂, H5′), 3.16-3.02 (m, 2H, CH₂, H1″), 2.93-2.75 (m, 2H, CH₂, H3″), 1.54 (s, 3H, CH₃, acetonide), 1.47 (s, 11H, CH₂, CH₃, H2″, t-butyl), 1.34 (s, 9H, CH₃, t-butyl), 1.33 (s, 3H, CH₃, acetonide).

### tert-butyl (3-aminopropyl)(((3αR,4R,6R,6αR)-6-(6-((tert-butoxycarbonyl)amino)-9H-purin-9-yl)-2,2-dimethyltetrahydrofuro[3,4-d][1,3]dioxol-4-yl)methyl)carbamate (114)

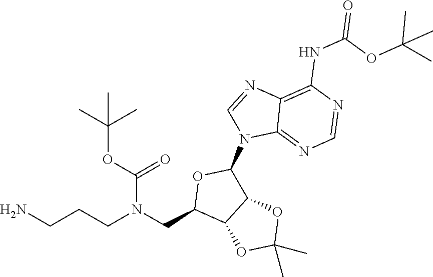 [SMILES: CC(C)(C)OC(=O)Nc1ncnc2c1ncn2[C@@H]1O[C@H](CN(CCCN)C(=O)OC(C)(C)C)[C@H]2OC(C)(C)O[C@H]21]

Nucleoside 113 (0.54 g, 0.79 mmol) was dissolved in a 10% piperidine solution in CH₂Cl₂ (7.80 mL). The reaction mixture was stirred for 24 h at ambient temperature. After 24 h, the mixture was diluted with water. The organic phase was separated, and the aqueous phase was extracted with CH₂Cl₂. The combined organic layers were dried over sodium sulfate and concentrated to complete dryness. The crude product was purified over silica (CH₂Cl₂/MeOH; 0-20%) to afford the pure product as colorless solid (0.44 g, 100%).

### Preparation of N₁-(((3αR,4R,6R,6αS)-2,2-dimethyl-6-(4-(methylamino)-7H-pyrrolo[2,3-d]pyrimidin-7-yl) tetrahydro-4H-cyclopenta[d][1,3]dioxol-4-yl)methyl)-2,2-difluoro-N₃-(2-(naphthalen-2-yl)ethyl)propane-1,3-diamine

### N-(((3αR,4R,6R,6a.S)-6-(4-chloro-7H-pyrrolo[2,3-a]pyrimidin-7-yl)-2,2-dimethyltetrahydro-4H-cyclopenta[d][1,3]dioxol-4-yl)methyl)-2,2-difluoro-N₃-(2-(naphthalen-2-yl)ethyl)propane-1,3-diamine (115)

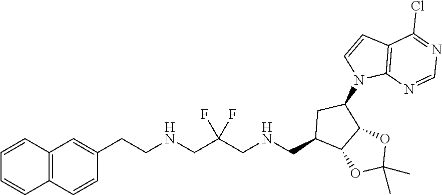 [SMILES: CC1(C)O[C@@H]2[C@@H](CNCC(F)(F)CNCCc3ccc4ccccc4c3)C[C@@H](n3ccc4c(Cl)ncnc43)[C@@H]2O1]

A solution of 2,2-difluoro-N-(2-(naphthalen-2-yl)ethyl)propane-1,3-diamine (25 mg, 0.09 mmol), (3αR,45,6R,6αS)-6-(4-chloro-7H-pyrrolo[2,3-d]pyrimidin-7-yl)-2,2-dimethyltetrahydro-4H-cyclopenta[d][1,3]dioxole-4-carbaldehyde (30 mg, 0.09 mmol) in MeOH (3 mL) was stirred at room temperature for 20 min. NaBH₃CN (22 mg, 0.35 mmol) and AcOH (2 drops) was added. The solution was stirred at room temperature for 16 hrs. The resulting mixture was concentrated. The residue was purified via flash with the solvent of ACN in H₂O. The collected fraction was concentrated. This afforded the desired product 8 (30 mg, yield: 57%) as a white solid. MS Calc.: 569.2; MS Found: 570.0 [M+H⁺].

### N-(((3αR,4R,6R,6a.S)-2,2-dimethyl-6-(4-(methylamino)-7H-pyrrolo[2,3-a]pyrimidin-7-yl) tetrahydro-4//-cyclopenta[d][1,3]dioxol-4-yl)methyl)-2,2-difluoro-N₃-(2-(naphthalen-2-yl)ethyl)propane-1,3-diamine (116)

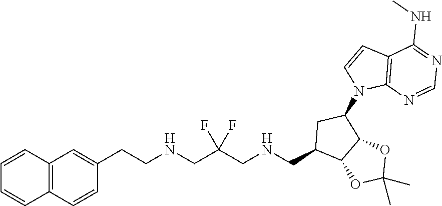 [SMILES: CNc1ncnc2c1ccn2[C@@H]1C[C@H](CNCC(F)(F)CNCCc2ccc3ccccc3c2)[C@H]2OC(C)(C)O[C@H]21]

A mixture of 115 (30 mg, 0.05 mmol) in methylamine in MeOH (5 mL) was stirred at room temperature for 16 hrs. The reaction solution was diluted with water (30 mL), extracted with EA (15 mL×3). The combined organic layer was dried with Na₂SO₄, filtered and concentrated. This afforded the crude desired product 116 (30 mg) as colorless oil. The crude product was used for next step without further purification.

### Preparation of tert-butyl ((E)-5-((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) pent-4-en-1-yl)(phenethyl)carbamate

### 3-((tert-butoxycarbonyl)(phenethyl) amino)propyl methanesulfonate (117)

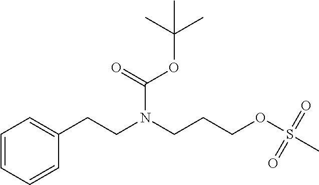 [SMILES: CC(C)(C)OC(=O)N(CCCOS(C)(=O)=O)CCc1ccccc1]

To a solution of tert-butyl (3-hydroxypropyl)(phenethyl)carbamate (1.0 g, 3.4 mmol), TEA (1.2 g, 10.2 mmol) in DCM (20 mL) was added MsCl (534 mg, 5.1 mmol) at 0° C., and the solution was stirred at room temperature for 2 hrs. The reaction solution was diluted with DCM (100 mL). The organic phase was washed with H₂O (100 mL), dried over Na₂SO₄ and concentrated to dryness. The crude was purified by flash (A: H₂O; B: MeCN) to give 117 (1.2 g, crude) as a clear oil. MS Calc.: 371.2; MS Found: 316.0 [M+H⁺-56].

### tert-butyl (3-iodopropyl)(phenethyl)carbamate (118)

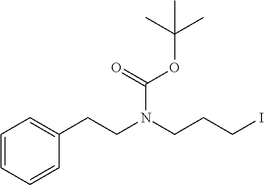 [SMILES: CC(C)(C)OC(=O)N(CCCI)CCc1ccccc1]

A solution of 117 (1.2 g, 3.4 mmol) and Nal (1.0 g, 6.8 mmol) in acetone (20 mL) was stirred at 40° C. for 2 hrs. The reaction solution was concentrated and the crude was purified by flash (A: H₂O; B: MeCN) to give 118 (600 mg, two step yield: 48%) as a clear oil. MS Calcd.: 403.1; MS Found: 347.8 [M+H⁺-56].

### (4-((tert-butoxycarbonyl)(phenethyl) amino) butyl) triphenylphosphonium iodide (119)

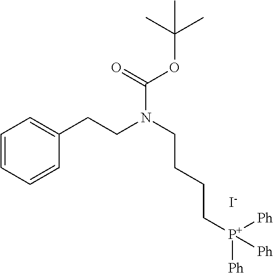 [SMILES: CC(C)(C)OC(=O)N(CCCC[P+](c1ccccc1)(c1ccccc1)c1ccccc1)CCc1ccccc1 ; [I-]]

A solution of 118 (300 mg, 0.74 mmol) and PPh₃ (215 mg, 0.82 mmol) in toluene (10 mL) was stirred at 110° C. for 24 hrs. The reaction mixture was hold still at room temperature for 2 hrs. The supernatant was removed and the remaining was concentrated to give 119 (300 mg, yield: 75%) as a white solid. MS Calcd.: 538.3; MS Found: 538.1 [M+].

### ((3αR,4R,6R,6a S)-6-(4-((4-methoxybenzyl)(methyl) amino)-7//pyrrolo[2,3-a]pyrimidin-7-yl)-2,2-dimethyltetrahydro-4H-cyclopenta[d][1,3]dioxol-4-yl) methanol (120)

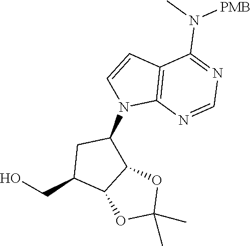

A solution of ((3αR,4R,6R,6αS)-6-(4-chloro-7H-pyrrolo[2,3-a]pyrimidin-7-yl)-2,2-dimethyltetrahydro-4H-cyclopenta[d][1,3]dioxol-4-yl) methanol (100 mg, 0.31 mmol), 1-(4-methoxyphenyl)-N-methylmethanamine (94 mg, 0.62 mmol) and TEA (94 mg, 0.93 mmol) in DMF (5 mL) was stirred at 70° C. for 24 hrs. The crude was purified by flash (A: H₂O; B: MeCN) to give 120 (100 mg, yield: 73%) as a clear oil. MS Calc.: 438.2; MS Found: 439.0 [M+H⁺].

### (3αR,4S,6R,6αS)-6-(4-((4-methoxybenzyl)(methyl) amino)-7//pyrrolo[2,3-d]pyrimidin-7-yl)-2,2-dimethyltetrahydro-4H-cyclopenta[d][1,3]dioxole-4-carbaldehyde (121)

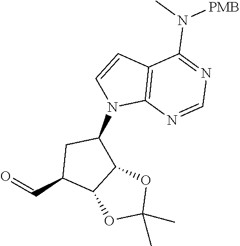

To a solution of 120 (100 mg, 0.23 mmol) in DCM (10 mL) was added Dess-Martin periodinane (134 mg, 0.34 mmol) and the solution was stirred at room temperature for 4 hrs. The reaction solution was diluted with DCM (20 mL). The organic phase was washed with H₂O (20 mL), aq. NαSO₃ (20 ml), aq. NaHCO₃ (20 ml) and dried over Na₂SO₄. The solution was concentrated to give 121 (100 mg, crude) as a brown oil.

### tert-butyl ((E)-5-((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) pent-4-en-1-yl)(phenethyl)carbamate (122)

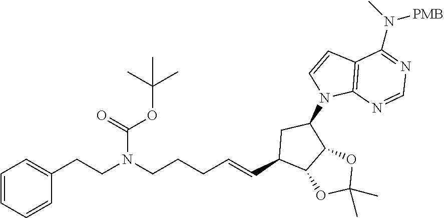

To a solution of 119 (200 mg, 0.37 mmol) in THF (5 ml), was added n-BuLi (0.15 ml, 0.37 mmol) at −78° C. and then was added 121 (100 mg, 0.23 mmol), the solution was stirred at room temperature for 2 hrs. The crude was purified by prep-HPLC(A: H₂O, TFA; B: MeCN) to give 122 (7 mg) as a white solid. MS Calcd.: 695.4; MS Found: 696.1 [M+H⁺]. Preparation of 7-((3αS,4R,6R,6αR)-2,2-dimethyl-6-((3-(((2-(naphthalen-2-yl)ethyl) amino) methyl) azetidin-1-yl)methyl)tetrahydro-4H-cyclopenta[d][1,3]dioxol-4-yl)-7H-pyrrolo[2,3-d]pyrimidin-4-amine

### tert-butyl 3-((2-(naphthalen-2-yl)acetamido) methyl) azetidine-1-carboxylate (123)

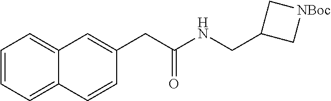 [SMILES: CC(C)(C)OC(=O)N1CC(CNC(=O)Cc2ccc3ccccc3c2)C1]

A suspension of 2-(naphthalen-2-yl)acetamide (300 mg, 1.61 mmol), tert-butyl 3-(aminomethyl) azetidine-1-carboxylate (300 mg, 1.61 mmol), HATU (735 mg, 1.94 mmol), TEA (325 mg, 3.22 mmol) in DMF (10 mL) was stirred at room temperature for 16 hrs under nitrogen atmosphere. The reaction mixture was diluted with EA (100 mL), washed with H₂O (50 mL×3) and dried over Na₂SO₄. The solution was concentrated and the crude was purified by flash (A: H₂O; B: MeCN) to give 123 (495 mg, yield: 87%) as a white solid. MS Calcd.: 354.1; MS Found: 355.0 [M+H⁺].

### tert-butyl 3-(((2-(naphthalen-2-yl)ethyl) amino) methyl) azetidine-1-carboxylate (124)

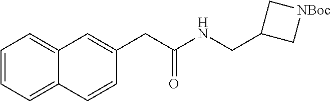 [SMILES: CC(C)(C)OC(=O)N1CC(CNC(=O)Cc2ccc3ccccc3c2)C1]

To a solution of 123 (425 mg, 1.2 mmol) in DCM (14 mL) was added DIBAL-H (6 mL, 6.0 mmol, 1M in THF) at −78° C., and the solution was stirred at room temperature for 16 hrs. The reaction was quenched with 10 ml of aq. NaHCO₃. The resulting solid was filtered. The filtrate was concentrated and the crude was purified by flash (A: H₂O, 0.1% TFA; B: MeCN) to give 124 (134 mg, yield: 33%) as a clear oil. MS Calcd.: 340.2; MS Found: 341.0 [M+H⁺].

### tert-butyl 3-((2,2,2-trifluoro-N-(2-(naphthalen-2-yl)ethyl)acetamido) methyl) azetidine-1-carboxylate (125)

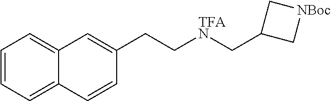

To a solution of 124 (134 mg, 0.394 mmol), TEA (119 mg, 1.182 mmol) in DCM (10 mL) was added TFAA (124 mg, 0.591 mmol) and the solution was stirred at room temperature for 1 hr. The resulting mixture was concentrated and the crude was purified by flash (A: H₂O, 0.1% TFA; B: MeCN) to give 125 (78 mg, yield: 45%) as a clear oil. MS Calcd.: 436.2; MS Found: 437.0 [M+H⁺].

### N-(azetidin-3-ylmethyl)-2.2.2-trifluoro-N-(2-(naphthalen-2-yl)ethyl)acetamide (126)

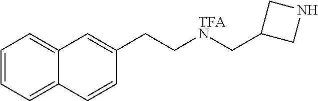

To a solution of 125 (78 mg, 0.179 mmol) in DCM (1.5 mL) was added TFA (1.5 mL), and the solution was stirred at room temperature for 1 hr. The mixture was concentrated to give 126 (80 mg, crude) as a clear oil. MS Calcd.: 336.1; MS Found: 337.0 [M+H⁺].

### N-((1-(((3αR,4R,6R,6αS)-6-(4-chloro-7H-pyrrolo[2,3-a]pyrimidin-7-yl)-2,2-dimethyltetrahydro-4H-cyclopenta[d][1,3]dioxol-4-yl)methyl) azetidin-3-yl)methyl)-2,2,2-trifluoro-N-(2-(naphthalen-2-yl)ethyl)acetamide (127)

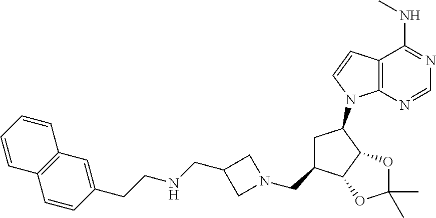 [SMILES: CNc1ncnc2c1ccn2[C@@H]1C[C@H](CN2CC(CNCCc3ccc4ccccc4c3)C2)[C@H]2OC(C)(C)O[C@H]21]

A solution of 126 (80 mg, 0.18 mmol) and (3αR,45,6R,6αS)-6-(4-chloro-7H-pyrrolo[2,3-d]pyrimidin-7-yl)-2,2-dimethyltetrahydro-4H-cyclopenta[d][1,3]dioxole-4-carbaldehyde (90 mg, 0.28 mmol) in MeOH (15 mL) was stirred at room temperature for 30 min. NaBH₃CN (88 mg, 0.54 mmol) and AcOH (1 drop) was added. The solution was stirred at room temperature for 16 hrs. The reaction mixture was purified by flash (A: H₂O; B: MeCN) after low temperature decompression and concentration to give 127 (104 mg, yield: 90%) as a clear oil. MS Calcd.: 641.2; MS Found: 642.0 [M+H⁺].

### 7-((3αS,4R,6R,6αR)-2,2-dimethyl-6-((3-(((2-(naphthalen-2-yl)ethyl) amino) methyl) azetidin-1-yl)methyl)tetrahydro-4//-cyclopenta[d][1,3]dioxol-4-yl)-N-methyl-7H-pyrrolo[2,3-a]pyrimidin-4-amine (128)

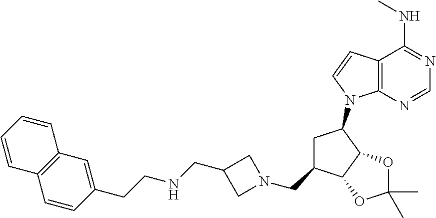 [SMILES: CNc1ncnc2c1ccn2[C@@H]1C[C@H](CN2CC(CNCCc3ccc4ccccc4c3)C2)[C@H]2OC(C)(C)O[C@H]21]

A solution of 127 (104 mg, 0.16 mmol) in MeNH₂ (6 mL) was stirred at 60° C. for 30 min. The resulting mixture was diluted with EA (50 mL), washed with H₂O (20 mL×2). The organic phase was dried over Na₂SO₄ and concentrated to give 128 (71 mg, yield: 81%) as a clear oil. MS Calcd.: 540.3; MS Found: 541.1 [M+H⁺].

### Synthesis of tert-butyl (7-((3αS,4R,6R,6αR)-6-(((3-((tert-butoxycarbonyl)(phenethyl) amino)propyl)(2,2,2-trifluoroethyl) amino)methyl)-2,2-dimethyltetrahydro-4H-cyclopenta[d][1,3]dioxol-4-yl)-7H-pyrrolo[2,3-d]pyrimidin-4-yl)(methyl)carbamate

### tert-butyl (7-((3αS,4R,6R,6αR)-2,2-dimethyl-6-(((2,2,2-trifluoroethyl) amino) methyl)tetrahydro-4H-cyclopenta[d][1,3]dioxol-4-yl)-7H-pyrrolo[2,3-d]pyrimidin-4-yl)(methyl)carbamate (129)

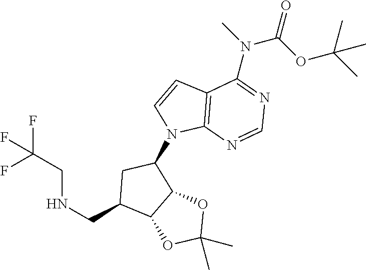 [SMILES: CN(C(=O)OC(C)(C)C)c1ncnc2c1ccn2[C@@H]1C[C@H](CNCC(F)(F)F)[C@H]2OC(C)(C)O[C@H]21]

Under inert (N₂) atmosphere, tert-butyl (7-((3αS,4R,6R,6αR)-6-(aminomethyl)-2,2-dimethyltetrahydro-4H-cyclopenta[d][1,3]dioxol-4-yl)-7H-pyrrolo[2,3-d]pyrimidin-4-yl)(methyl) carbamate (0.103 g, 0.25 mmol, 1.0 equiv.) was dissolved in 2 ml dry THF. Then, triethylamine (67 μl, 0.049 g, mmol, 1.9 equiv.) was added, followed by addition of 2,2,2-trifluoroethyl trifluoromethanesulfonate (69 μl, 0.11 g, 0.48 mmol, 1.9 equiv.). The reaction was then stirred overnight at room temperature. After 17.5 h, the solvent was evaporated and water was added. The mixture was extracted with DCM (3x), dried over Na₂SO₄ and after filtration and evaporation the residue was purified by flash chromatography eluting Cyclohexane/EtOAc (5% to 57%) to afford the product as a white foam (yield: 0.082 g, 0.16 mmol, 66%). ¹H NMR (400 MHZ, DMSO-d₆) δ 8.63 (s, 1H, H2), 7.75 (d, J=3.7 Hz, 1H, H8), 6.45 (d, J=3.7 Hz, 1H, H7), 5.09 (dt, J=12.7, 6.4 Hz, 1H, H1′), 4.94-4.88 (m,, 1H, H2′), 4.50 (dd, J=7.3, 4.9 Hz, 1H, H3′), 3.35 (s, 3H, —NH—CH₃), 3.27 (qd, J=10.3, 6.7 Hz, 2H), 2.86-2.77 (m, 1H, H5′A), 2.76-2.69 (m, 1H, H5′B), 2.49-2.39 (m, 1H, —NH—), 2.35-2.18 (m, 2H, H4′, H6′A), 2.07-1.96 (m, 1H, H6′B), 1.49 (s, 3H, CH₃, acetonide), 1.45 (s, 9H, t-Bu, Boc), 1.20 (s, 3H, CH₃, acetonide). C₂₃H32F3N₅₀₄ (499.54 g/mol). APCI: calcd. For C₂₃H32F3N₅₀₄ [M+H]⁺: 499.24, found: 499.7/399.7.

### Preparation of tert-butyl 2-(3-((tert-butoxycarbonyl)(3-phenoxyphenethyl) amino)propyl)-2-(((3αR,4R,6R,6αS)-2,2-dimethyl-6-(4-(methylamino)-7H-pyrrolo[2,3-d]pyrimidin-7-yl) tetrahydro-4H-cyclopenta[d][1,3]dioxol-4-yl)methyl)-1-methylhydrazine-1-carboxylate

### tert-butyl 1-(3-((tert-butoxycarbonyl)(3-phenoxyphenethyl) amino)propyl)-2-methylhydrazine-1-carboxylate (130)

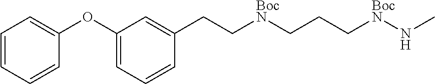 [SMILES: CNN(CCCN(CCc1cccc(Oc2ccccc2)c1)C(=O)OC(C)(C)C)C(=O)OC(C)(C)C]

A mixture of compound tert-butyl (3-oxopropyl)(3-phenoxyphenethyl)carbamate (200 mg, 0.54 mmol) and tert-butyl 1-methylhydrazinecarboxylate (157 mg, 1.08 mmol) in MeOH (3 mL) was stirred at room temperature for 1 hr. Then NaBH₃CN (170 mg, 2.7 mmol) was added. The resulting mixture was stirred for additional 1 hr and purified by flash chromatography (50% EA in PE) to give compound 130 (100 mg, yield: 37%) as a yellow solid. MS Calc.: 499.3; MS Found: 500.3 [M+H]⁺.

### (3αR,45,6R,6αS)-2,2-dimethyl-6-(4-(methylamino)-7H-pyrrolo[2,3-d]pyrimidin-7-yl) tetrahydro-4H-cyclopenta[d][1,3]dioxole-4-carbaldehyde (131)

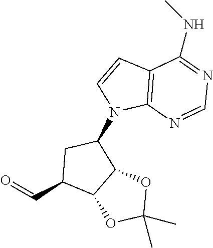 [SMILES: CNc1ncnc2c1ccn2[C@@H]1C[C@H](C=O)[C@H]2OC(C)(C)O[C@H]21]

To a solution of ((3αR,4R,6R,6αS)-2,2-dimethyl-6-(4-(methylamino)-7H-pyrrolo[2,3-d]pyrimidin-7-yl) tetrahydro-4H-cyclopenta[d][1,3]dioxol-4-yl) methanol (100 mg, 0.23 mmol) in DCM (10 mL) was added Dess-Martin periodinane (134 mg, 0.34 mmol) and the solution was stirred at room temperature for 4 hrs. The reaction solution was diluted with DCM (20 mL). The organic phase was washed with H₂O (20 mL), aq. NαSO₃ (20 ml), aq. NaHCO₃ (20 ml) and dried over Na₂SO₄. The solution was concentrated to give 131 (100 mg, crude) as a brown oil.

### tert-butyl 2-(3-((tert-butoxycarbonyl)(3-phenoxyphenethyl) amino)propyl)-2-(((3αR,4R,6R,6a.S)-2,2-dimethyl-6-(4-(methylamino)-7H-pyrrolo[2,3-a]pyrimidin-7-yl) tetrahydro-4H-cyclopenta[d][1,3]dioxol-4-yl)methyl)-1-methylhydrazine-1-carboxylate (132)

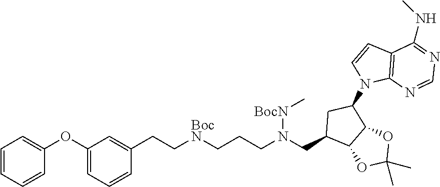 [SMILES: CNc1ncnc2c1ccn2[C@@H]1C[C@H](CN(CCCN(CCc2cccc(Oc3ccccc3)c2)C(=O)OC(C)(C)C)N(C)C(=O)OC(C)(C)C)[C@H]2OC(C)(C)O[C@H]21]

A mixture of compound 130 (100 mg, 0.2 mmol) and 131 (63.5 mg, 0.2 mmol) in MeOH (3 mL) was stirred at room temperature for 1 hr. Then NaBH₃CN (120 mg, 2.0 mmol) was added. The resulting mixture was stirred for additional 1 h and purified by prep-HPLC(TFA buffer) to give compound 132 (40 mg, yield: 25%) as a white solid.

### Synthesis of tert-butyl (3-((((3αR,4R,6R,6αS)-2,2-dimethyl-6-(4-(methylamino)-7H-pyrrolo[2,3-d]pyrimidin-7-yl) tetrahydro-4H-cyclopenta[d][1,3]dioxol-4-yl)methyl)(ethyl) amino)propyl)(2-(naphthalen-2-yl)ethyl)carbamate

### tert-butyl (3-((((3αR,4R,6R,6αS)-2,2-dimethyl-6-(4-(methylamino)-7H-pyrrolo[2,3-d]pyrimidin-7-yl) tetrahydro-4H-cyclopenta[d][1,3]dioxol-4-yl)methyl) amino)propyl)(2-(naphthalen-2-yl)ethyl)carbamate (133)

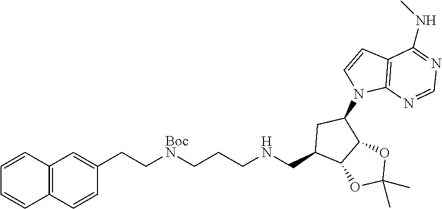 [SMILES: CNc1ncnc2c1ccn2[C@@H]1C[C@H](CNCCCN(CCc2ccc3ccccc3c2)C(=O)OC(C)(C)C)[C@H]2OC(C)(C)O[C@H]21]

To a solution of 131 (50 mg, 0.16 mmol) and HOAc (0.1 mL) in MeOH (2 mL) was added compound N-(2-(naphthalen-2-yl)ethyl)propane-1,3-diamine (65 mg, 0.2 mmol)), and the mixture was stirred at room temperature for 0.1 h. Then NaBH₃CN (99 mg, 1.6 mmol) was added and the mixture was stirred at room temperature for 1 hr. The mixture was used for next step. MS Calc.: 628.4; MS Found: 629.2 [M+H]⁺

### tert-butyl (3-((((3αR,4R,6R,6αS)-2,2-dimethyl-6-(4-(methylamino)-7H-pyrrolo[2,3-d]pyrimidin-7-yl) tetrahydro-4H-cyclopenta[d][1,3]dioxol-4-yl)methyl)(ethyl) amino)propyl)(2-(naphthalen-2-yl)ethyl)carbamate (134)

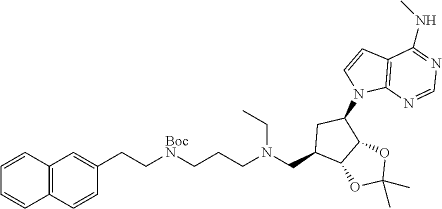 [SMILES: CCN(CCCN(CCc1ccc2ccccc2c1)C(=O)OC(C)(C)C)C[C@H]1C[C@@H](n2ccc3c(NC)ncnc32)[C@@H]2OC(C)(C)O[C@H]12]

To a solution of the last step was added acetaldehyde (0.2 mL), and the mixture was stirred at room temperature for 2 hrs. The compound was found by LC-MS. The reaction was quenched with water (0.5 mL). The solution was concentrated to dryness and the residue was purified by prep-HPLC(NH₄OAc buffer) to give compound 134 (30 mg, two-step yield: 28.5%) as an off-white solid. MS Calc.: 656.4; MS Found: 657.4 [M+H]⁺.

### Synthesis of (3αS,45,6R,6αR)-6-(6-amino-9H-purin-9-yl)-2,2-dimethyltetrahydrofuro[3,4-d][1,3]dioxole-4-carboxylic acid

### (3αS,45,6R,6αR)-6-(6-amino-9H-purin-9-yl)-2,2-dimethyltetrahydrofuro[3,4-d][1,3]dioxole-4-carboxylic acid (135)

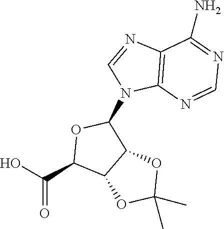 [SMILES: CC1(C)O[C@@H]2[C@H](O1)[C@@H](C(=O)O)O[C@H]2n1cnc2c(N)ncnc21]

2,3-O-ispropylideneadenosine (2.10 g, 6.77 mmol), BAIB (4.84 g, 14.88 mmol), and TEMPO (0.21 g, 1.35 mmol) were added to a 100 mL flask. Then, this solid mixture was dissolved in a 1:1 mixture of MeCN/H₂O (33.80 mL) and stirred magnetically at rt for 4 h. During this time a precipitate was formed which was filtered off. The filter cake was rinsed with additional MeCN and dried to complete dryness which afforded the pure product as white solid (1.85 g, 85%). 1H NMR (400 MHZ, DMSO-d₆) δ 12.82 (s, 1H, COOH), 8.25 (s, 1H, H2), 8.09 (s, 1H, H8), 7.30 (s, 2H, N₆—NH₂), 6.34 (s, 1H, H1′), 5.55 (dd, J=6.0, 2.0 Hz, 1H, H3′), 5.47 (d, J=6.0 Hz, 1H, H2′), 4.70 (d, J=1.9 Hz, 1H, H4′), 1.53 (s, 3H, CH₃, acetonide), 1.36 (s, 3H, CH₃, acetonide).

### Synthesis of (3αR,45,6R,6αS)-6-(4-chloro-7H-pyrrolo[2,3-d]pyrimidin-7-yl)-2,2-dimethyltetrahydro-4H-cyclopenta[d][1,3]dioxole-4-carboxylic acid

### (3αR,45,6R,6αS)-6-(4-chloro-7H-pyrrolo[2,3-d]pyrimidin-7-yl)-2,2-dimethyltetrahydro-4H cyclopenta[d][1,3]dioxole-4-carboxylic acid (136)

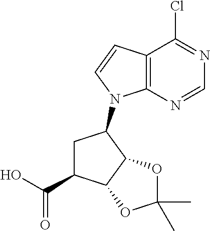 [SMILES: CC1(C)O[C@@H]2[C@H](O1)[C@@H](C(=O)O)C[C@H]2n1ccc2c(Cl)ncnc21]

To a solution of ((3αR,4R,6R,6αS)-6-(4-chloro-7H-pyrrolo[2,3-d]pyrimidin-7-yl)-2,2-dimethyltetrahydro-4H-cyclopenta[d][1,3]dioxol-4-yl) methanol (2.85 g, 8.8 mmol) in DCM (130 mL) was added Dess-Martin periodinane (9.7 g, 23 mmol) at 0° C. under N₂. The mixture was stirred at room temperature for 24 h. The reaction was quenched with saturated NaHCO₃ solution (65 mL) and stirred for around 15 min. The organic layer was separated, dried over Na₂SO₄ and concentrated in vacuum to afford a crude product, which was purified by reverse phase flash (MeCN/H₂O)(330 mg, 11% yield) to afford compound 136 as a white solid. MS Calc.: 337.1; MS Found: 338.3 [M+H]+

### (3αR,45,6R,6αS)-2,2-dimethyl-6-(4-(methylamino)-7H-pyrrolo[2,3-a]pyrimidin-7-yl) tetrahydro-4H-cyclopenta[d][1,3]dioxole-4-carboxylic acid (137)

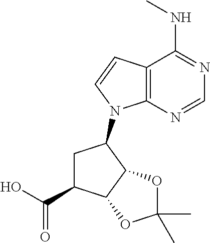 [SMILES: CNc1ncnc2c1ccn2[C@@H]1C[C@H](C(=O)O)[C@H]2OC(C)(C)O[C@H]21]

To a solution of compound 136 (330 mg, 1.0 mmol) in MeOH (5 mL) was added CH₃NH₂/C₂H₅OH (5 mL), the reaction mixture was stirred at r. t. for 0.5 hr, followed by concentration. The residue was acidified with HCl (6M) and purified by reverse phase flash (MeCN/H₂O) to afford compound 137 (200 mg, 68% yield) as a white solid. MS Calc.: 292.1; MS Found: 293.1 [M+H]⁺.

### Preparation of tert-butyl (9-((3αR,4R,6R,6αR)-6-(aminomethyl)-2,2-dimethyltetrahydrofuro[3,4-d][1,3]dioxol-4-yl)-9H-purin-6-yl)carbamate

### tert-butyl (9-((3αR,4R,6R,6αR)-6-(hydroxymethyl)-2,2-dimethyltetrahydrofuro[3,4-d][1,3]dioxol-4-yl)-9H-purin-6-yl)carbamate (138)

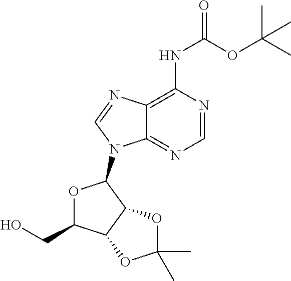 [SMILES: CC(C)(C)OC(=O)Nc1ncnc2c1ncn2[C@@H]1O[C@H](CO)[C@H]2OC(C)(C)O[C@H]21]

In an oven-dried three-necked round bottom flask 2′,3′-O-Isopropylideneadenosine (4.00 g, 12.89 mmol) was suspended in hexamethyldisilazane (5.47 mL, 25.77 mmol) under nitrogen, Then, DMAP (0.40 g, 3.22 mmol) was added followed by addition of TMSOTf (0.05 mL, 0.26 mmol) at rt. The suspension was heated to 75° C. and stirred for 2 h at this temperature. Afterwards, the mixture was concentrated to complete dryness. The obtained residue was dissolved in dry THF (43.00 mL) and cooled down in an ice-bath. To this cooled solution di-t-butyl decarbonate (8.36 mL, 38.66 mmol) was added portion wise. The mixture was stirred overnight at ambient temperature. The solution was concentrated under reduced pressure. The obtained residue was re-dissolved in EtOH (43.00 mL) and then treated with a 33% MeNH₂ solution in EtOH (5.28 mL, 4.00 eq). The solution was heated to 50° C. and stirred for 48 h at this temperature. Then, the reaction mixture was concentrated under reduced pressure. The residue was diluted with water and the aqueous phase was extracted with CH₂Cl₂. The combined organic layers were dried over sodium sulfate and concentrated to an oil. The crude product was purified over silica (cyclohexane/EtOAc; 0-100%) to afford a colorless foam (3.88 g, 74%). Analytical data according to literature data.

### tert-butyl (9-((3αR,4R,6R,6αR)-6-(azidomethyl)-2,2-dimethyltetrahydrofuro[3,4-d][1,3]dioxol-4-yl)-9H-purin-6-yl)carbamate (139)

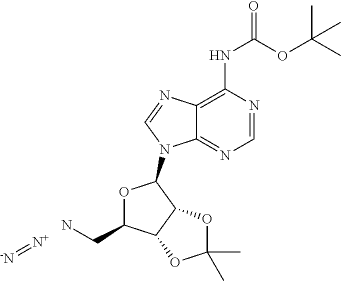

To a stirred solution of compound 138 (1.75 g, 4.30 mmol) in CH₂Cl₂ (21.50 mL) triethylamine (0.90 mL, 6.44 mmol) and MsCl (0.40 mL, 5.15 mmol) were added at 0° C. under nitrogen atmosphere. After 30 min stirring at rt, the solution was diluted with brine. The organic layer was separated, and the aqueous phase was extracted with CH₂Cl₂. The combined organic layers were dried over sodium sulfate, filtered, and concentrated to complete dryness. The crude product was redissolved in DMF and sodium azide (0.85 g, 12.87 mmol) was added. The suspension was heated to 60° C. and stirred for 5 h. Then, the organic solvent was evaporated. Water was added to the obtained residue and the aqueous phase was extracted with EtOAc. The combined organic layers were washed with brine, dried over sodium sulfate, and concentrated. The crude product was purified by flash chromatography (cyclohexane/EtOAc; 0-80%) to afford the product as colorless foam (1.27 g, 69%). Analytical data according to literature data.

### tert-butyl (9-((3αR,4R,6R,6αR)-6-(aminomethyl)-2,2-dimethyltetrahydrofuro[3,4-d][1,3]dioxol-4-yl)-9H-purin-6-yl)carbamate (140)

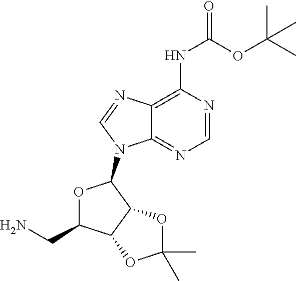 [SMILES: CC(C)(C)OC(=O)Nc1ncnc2c1ncn2[C@@H]1O[C@H](CN)[C@H]2OC(C)(C)O[C@H]21]

Compound 139 (1.25 g, 2.86 mmol) was dissolved in a mixture of EtOH and MeOH (2:1, 38.60 mL). The solution was degassed and stirred at rt under nitrogen. Then, palladium on activated charcoal moistened with water (0.61 g, 0.29 mmol) was added and the mixture was again degassed. Afterwards, the reaction mixture was purged with H2 from a storage vessel and stirred overnight at ambient temperature. Then the suspension was purged with nitrogen and filtered off over Celite. The filter cake was rinsed with EtOH and CH₂Cl₂. The filtrate was concentrated under reduced pressure and the obtained residue was purified over silica ( ) which afforded the product as colorless foam (0.90 g, 85%). Analytical data according to literature data. Preparation of tert-butyl (7-((3αR,4R,6R,6αR)-6-(aminomethyl)-2,2dimethyltetrahydrofuro[3,4-d][1,3]dioxol-4-yl)-7H-pyrrolo[2,3-d]pyrimidin-4-yl)(methyl)carbamate

### (2R,3R,4R,5R)-2-((benzoyloxy) methyl)-5-(4-chloro-5-iodo-7H-pyrrolo[2,3-d]pyrimidin-7-yl) tetrahydrofuran-3,4-diyl dibenzoate (141)

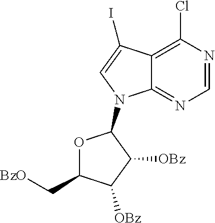

In a heat-dried three-necked round bottom flask was added the nucleobase (2.89 g, 10.34 mmol) in dry MeCN (51.70 mL) under nitrogen. Then, BSA (2.81 mL, 11.38 mmol) was added to the stirred suspension. The suspension was stirred for 10 minutes. During this time the reaction became a homogenous solution. Then, the sugar (5.80 g, 11.38 mmol) was added, and the reaction mixture was heated to 85° C. for 2 h. After this the reaction was cooled down to rt before diluted with EtOAc (100 mL). The organic phase was washed with saturated bicarbonate solution (1×100 mL) and with brine (1×100 mL). The combined organic layers were dried over sodium sulfate, filtered, and concentrated under reduced pressure. The crude product was purified over silica (cyclohexane/EtOAc; 0-40%) to afford the product as yellowish foam. (5.24 g, 64%). ¹H NMR (400 MHZ, DMSO-d₆) δ8.60 (s, 1H, H2), 8.30 (s, 1H, H5), 8.01-7.83 (m, 6H, o-Ar, Bz), 7.71-7.60 (m, 3H, p-Ar, Bz), 7.55-7.40 (m, 6H, m-Ar, Bz), 6.71 (d, J=5.0 Hz, 1H, H1′), 6.29 (dd, J=6.1, 5.1 Hz, 1H, H2′), 6.14 (t, J=5.8 Hz, 1H, H3′), 4.87 (q, J=5.0 Hz, 1H, H4′), 4.83-4.64 (m, 2H, H5′).

### (2R,35,4R,5R)-2-(hydroxymethyl)-5-(5-iodo-4-(methylamino)-7H-pyrrolo[2,3-d]pyrimidin-7-yl) tetrahydrofuran-3,4-diol(142)

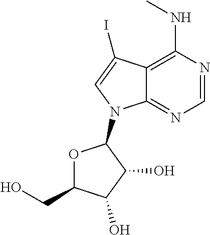 [SMILES: CNc1ncnc2c1c(I)cn2[C@@H]1O[C@H](CO)[C@@H](O)[C@H]1O]

The nucleoside 141 (7.08 g, 9.68 mmol) was dissolved in 1,4-dioxane (96.90 mL) and 33% MeNH₂ solution in EtOH (96.90 mL) in a sealed flask under nitrogen. The solution was heated to 60° C. overnight. Then, the reaction was allowed to cool down to rt before concentrated under reduced pressure. The obtained oil was purified by flash chromatography (CH₂Cl₂/MeOH; 0-20%) which afford the product as slightly yellowish foam (3.02 g, 77%). ¹H NMR (400 MHZ, DMSO-d₆) δ 8.19 (s, 1H, H2), 7.66 (s, 1H, H6), 6.44 (q, J=4.6 Hz, 1H, NH), 6.03 (d, J=6.3 Hz, 1H, H1′), 5.31 (d, J=6.5 Hz, 1H, 2′OH), 5.16 (t, J=5.6 Hz, 1H, 5′OH), 5.12 (d, J=4.8 Hz, 1H, 3′OH), 4.38-4.32 (m, 1H, H2′), 4.06 (td, J=4.8, 3.2 Hz, 1H, H3′), 3.88 (q, J=3.7 Hz, 1H, H4′), 3.57 (m, 2H, H5′), 3.02 (d, J=4.7 Hz, 3H, CH₃, NMe).

((3αR,4R,6R,6αR)-2,2-dimethyl-6-(4-(methylamino)-7H-pyrrolo[2,3-d]pyrimidin-7-yl) tetrahydrofuro[3,4-d][1,3]dioxol-4-yl) methanol (143)

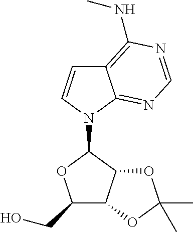 [SMILES: CNc1ncnc2c1ccn2[C@@H]1O[C@H](CO)[C@H]2OC(C)(C)O[C@H]21]

Compound 142 (3.00 g, 7.31 mmol) was dissolved in dry DMF (36.60 mL) under nitrogen. Then, Et₃N (2.05 mL, 14.62 mmol) was added under nitrogen before palladium on activated charcoal moistened with water (1.56 g, 0.73 mmol) was added. The reaction mixture was purged with hydrogen from a storage vessel and was magnetically stirred overnight. At this point TLC (CH₂Cl₂/MeOH; 10%) indicated full conversion of the starting. Then, the reaction mixture was purged with nitrogen followed by filtration over Celite. The filter cake was rinsed with EtOH and the filtrate was concentrated over vacuum. The dehalogenated compound (2.05 g, 7.24 mmol) was suspended in acetone (36.60 mL). To the stirred suspension triethoxymethane (6.09 mL, 36.19 mmol) and p-TsOH (6.95 g, 36.19 mmol) were added sequentially at rt. The suspension became a clear yellow solution and was stirred overnight at rt. Then, the mixture was quenched by 5% aqueous sodium bicarbonate solution. The organic solvent was removed under reduced pressure. The aqueous phase was extracted with CH₂Cl₂. The combined organic layers were washed with brine, dried over sodium sulfate, and concentrated over vacuum. The obtained residue was purified by flash chromatography (CH₂Cl₂/MeOH; 0-5%) which afforded the product as a colorless foam (2.24 g, 97%). ¹H NMR (400 MHZ, DMSO-d₆) δ8.16 (s, 1H, H2), 7.61-7.54 (m, 1H, NH), 7.35 (d, J=3.6 Hz, 1H, H6), 6.60 (d, J=3.5 Hz, 1H, H5), 6.16 (d, J=3.6 Hz, 1H, H1′), 5.23 (t, J=5.6 Hz, 1H, 5′OH), 5.15 (dd, J=6.2, 3.7 Hz, 1H, H2′), 4.90 (dd, J=6.3, 2.8 Hz, 1H, H3′), 4.15-4.09 (m, 1H, H4′), 3.60-3.48 (m, J=5.2 Hz, 2H, H5′), 2.95 (d, J=4.5 Hz, 3H, CH₃, NMe), 1.54 (s, 3H, CH₃, acetonide), 1.31 (s, 3H, CH₃, acetonide).

### 7-((3αR,4R,6R,6αR)-6-(azidomethyl)-2,2-dimethyltetrahydrofuro[3,4-d][1,3]dioxol-4-yl)-N-methyl-7H-pyrrolo[2,3-d]pyrimidin-4-amine (144)

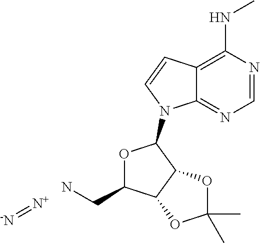

Compound 143 (2.23 g, 6.88 mmol) was dissolved in dry 1,4-dioxane (34.40 mL) and then cooled down in an ice-bath. Afterwards, DPPA (2.99 mL, 13.75 mmol) and DBU (3.11 mL, 20.63 mmol) were added to the cooled solution and stirred for 24 h at rt. Then, 15-crown-5 (1.44 mL, 6.88 mmol) and NaN₃ (2.26 g, 34.38 mmol) were added, and the suspension was heated to 110° C. After 6 h, the reaction mixture was allowe to cool down to rt and then the organic solvent was evaporated. The obtained oil was diluted with water and the aqueous phase was extracted three times with EtOAc. The combined organic layers were washed with brine, dried over sodium sulfate, and concentrated under reduced pressure. The crude product was purified over silica (cyclohexane/EtOAc; 0-100%) to obtain the desired product as colorless foam (1.06 g, 45%). ¹H NMR (400 MHZ, DMSO-d₆) δ8.18 (s, 1H, H2), 7.58 (q, J=4.0 Hz, 1H, NH), 7.34 (d, J=3.7 Hz, 1H, H6), 6.62 (d, J=3.6 Hz, 1H, H5), 6.23 (d, J=3.2 Hz, 1H, H1′), 5.31 (dd, J=6.4, 3.2 Hz, 1H, H2′), 4.93 (dd, J=6.4, 3.4 Hz, 1H, H3′), 4.21 (td, J=5.6, 3.4 Hz, 1H, H4′), 3.57-3.54 (m, 2H, H5′), 2.95 (d, J=4.6 Hz, 3H, CH₃, NMe), 1.54 (s, 3H, CH₃, acetonide), 1.32 (s, 3H, CH₃, acetonide).

### tert-butyl (7-((3αR,4R,6R,6αR)-6-(azidomethyl)-2,2-dimethyltetrahydrofuro[3,4-d][1,3]dioxol-4-yl)-7H-pyrrolo[2,3-d]pyrimidin-4-yl)(methyl)carbamate (145)

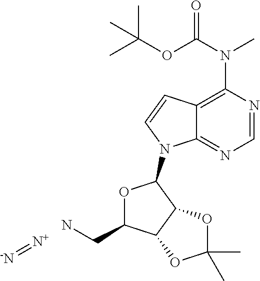

Compound 144 (1.04 g, 2.99 mmol) was dissolved in dry THF (13.00 mL). The solution was cooled down before NaH (60% in mineral oil, 1.79 g, 44.81 mmol) was added portion wise. The suspension was stirred for 45 min at rt before cooling down to 0° C. To the cooled suspension, di-tert butyl dicarbonate (4.16 mL, 17.92 mmol) was added dropwise. After 3 days TLC (CH₂Cl₂/MeOH; 5%) indicated poor conversion. At this point dry DMF (3.00 mL) was added, and the mixture was stirred for 6 h at rt. TLC(CH₂Cl₂/MeOH; 5%) indicated almost full conversion. Then, the reaction mixture was cooled down in an ice-bath and then carefully quenched with dropwise addition of water. The aqueous phase was extracted with CH₂Cl₂. The combined organic layers were washed with brine, dried over sodium sulfate, and concentrated under reduced pressure. The obtained residue was purified over silica (cyclohexane/EtOAc; 0-100%) which afforded the product as colorless foam (0.82 g, 62%). ¹H NMR (400 MHZ, DMSO-d₆) δ8.67 (s, 1H, H2), 7.76 (d, J=3.8 Hz, 1H, H6), 6.50 (d, J=3.8 Hz, 1H, H5), 6.37 (d, J=3.2 Hz, 1H, H1′), 5.36 (dd, J=6.4, 3.2 Hz, 1H, H2′), 4.97 (dd, J=6.4, 3.4 Hz, 1H, H3′), 4.30-4.24 (m, 1H, H4′), 3.63-3.51 (m, 2H, H5′), 3.36 (s, 3H, CH₃. N-Me), 1.56 (s, 3H, CH₃, acetonide), 1.44 (s, 9H, CH₃, t-butyl), 1.33 (s, 3H, CH₃, acetonide).

### tert-butyl (7-((3αR,4R,6R,6αR)-6-(aminomethyl)-2,2-dimethyltetrahydrofuro[3,4-d][1,3]dioxol-4-yl)-7H-pyrrolo[2,3-d]pyrimidin-4-yl)(methyl)carbamate (146)

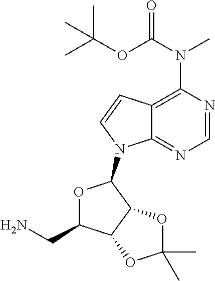 [SMILES: CN(C(=O)OC(C)(C)C)c1ncnc2c1ccn2[C@@H]1O[C@H](CN)[C@H]2OC(C)(C)O[C@H]21]

The azide 145 (0.81 g, 1.81 mmol) was dissolved in a 4:1 EtOH/MeOH mixture (18.00 mL) under nitrogen. Then, palladium on activated charcoal moistened with water (0.38 g, 0.18 mmol) was added at rt. Then, the mixture was purged with H2 from a storage vessel and stirred overnight at rt. TLC(CH₂Cl₂/MeOH; 5%) monitored full consumption of the SM. Then, the mixture was purged with nitrogen and filtered over Celite. The filter cake was rinsed with EtOH and the filtrate was concentrated under reduced pressure. The crude product was purified over silica (CH₂Cl₂/MeOH; 0-5%) which afforded the product as colorless foam (0.47 g, 63%). ¹H NMR (400 MHZ, DMSO-d₆) δ8.66 (s, 1H, H2), 7.78 (d, J=3.8 Hz, 1H, H6), 6.48 (d, J=3.7 Hz, 1H, H5), 6.29 (d, J=3.7 Hz, 1H, H1′), 5.29 (dd, J=6.4, 3.7 Hz, 1H, H2′), 4.96 (dd, J=6.4, 3.1 Hz, 1H, H3′), 4.09-4.03 (m, 1H, H4′), 3.37 (s, 3H, CH₃, N-Me), 2.73 (dd, J=5.6, 3.9 Hz, 2H, H5′), 1.56 (s, 3H, CH₃, acetonide), 1.45 (s, 9H, CH₃, t-butyl), 1.33 (s, 3H, CH₃, acetonide).

### Preparation of tert-butyl (7-((3a5,4R,6R,6αR)-6-(aminomethyl)-2,2-dimethyltetrahydro-4H-cyclopenta[d][1,3]dioxol-4-yl)-7H-pyrrolo[2,3-d]pyrimidin-4-yl)(methyl)carbamate

### (1R,2S,3R,5R)-3-(4-chloro-7H-pyrrolo[2,3-d]pyrimidin-7-yl)-5-(hydroxymethyl) cyclopentane-1,2-diol (147)

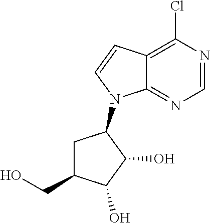 [SMILES: OC[C@H]1C[C@@H](n2ccc3c(Cl)ncnc32)[C@H](O)[C@@H]1O]

The 3-amino-5-(hydroxymethyl) cyclopentane-1,2-diol (5.00 g, 26.96 mmol) was dissolved in EtOH (234.40 mL). To this solution, 4,6-dichloropyrimidin-5-acetaldehyde (5.15 g, 26.96 mmol) and triethylamine (12.52 mL, 53.91 mmol) were added at ambient temperature. The reaction mixture was heated to 90° C. and stirred magnetically overnight. After 18 h, the volatiles were evaporated under reduces pressure and the obtained residue was purified over silica (CH₂Cl₂/MeOH; 0-10%) which afforded the product as yellowish solid (6.96 g, 91%). ¹H NMR (400 MHz, DMSO-d₆) δ 8.63 (s, 1H, H2), 7.91 (d, J=3.7 Hz, 1H, H6), 6.70 (d, J=3.6 Hz, 1H, H5), 5.05 (dt, J=10.4, 8.6 Hz, 1H, H1′), 4.87 (d, J=6.7 Hz, 1H, 2′OH), 4.76 (t, J=5.2 Hz, 1H, 5′OH), 4.70 (d, J=4.2 Hz, 1H, 3′OH), 4.24 (ddd, J=9.2, 6.6, 5.3 Hz, 1H, H2′), 3.84 (dq, J=4.9, 3.0 Hz, 1H, H3′), 3.55-3.43 (m, 2H, CH₂, H5′), 2.23 (dt, J=12.8, 8.7 Hz, 1H, CH₂, cyclopentane), 2.11-2.01 (m, 1H, H4′), 1.61 (ddd, J=12.9, 10.5, 7.9 Hz, 1H, CH₂, cyclopentane).

### ((3αR,4R,6R,6αS)-6-(4-chloro-7H-pyrrolo[2,3-d]pyrimidin-7-yl)-2,2-dimethyltetrahydro-4H-cyclopenta[d][1,3]dioxol-4-yl) methanol (148)

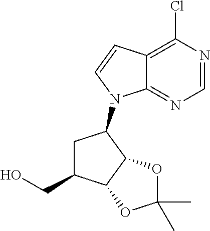 [SMILES: CC1(C)O[C@@H]2[C@@H](CO)C[C@@H](n3ccc4c(Cl)ncnc43)[C@@H]2O1]

To a suspension of compound 147 (4.21 g, 14.67 mmol) in acetone (391.30 mL), triethoxymethane (12.33 mL, 73.37 mmol) and pTsOH (14.10 g, 73.37 mmol) were added. The suspension became a clear solution and was stirred at ambient temperature for 20 h. Then, the reaction mixture was quenched with 5% aqueous sodium bicarbonate solution. The organic solvent was removed under reduced pressure. The aqueous phase was extracted with CH₂Cl₂. The combined organic layers were washed with brine, dried over sodium sulfate, and concentrated. The obtained residue was purified over silica (CH₂Cl₂/MeOH; 0-5%) to afford the product as yellowish foam (4.90 g, 62%). ¹H NMR (400 MHZ, DMSO-d₆) δ8.66 (s, 1H, H2), 7.97 (d, J=3.7 Hz, 1H, H6), 6.73 (d, J=3.7 Hz, 1H, H5), 5.10 (dt, J=13.0, 6.5 Hz, 1H, H1′), 4.92 (t, J=6.8 Hz, 1H, H2′), 4.82 (t, J=5.3 Hz, 1H, 5′OH), 4.55 (dd, J=7.2, 4.3 Hz, 1H, H3′), 3.53 (td, J=5.4, 1.3 Hz, 2H, CH₂, H5′), 2.31-2.20 (m, 1H, H4′, CH₂, cyclopentane), 2.14-2.09 (m, 1H, CH₂, cyclopentane), 1.49 (s, 3H, CH₃, acetonide), 1.23 (s, 3H, CH₃, acetonide).

### ((3αR,4R,6R,6αS)-2,2-dimethyl-6-(4-(methylamino)-7H-pyrrolo[2,3-d]pyrimidin-7-yl) tetrahydro-4H-cyclopenta[d][1,3]dioxol-4-yl) methanol (149)

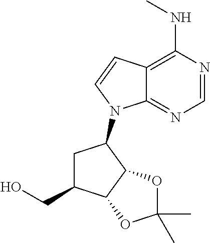 [SMILES: CNc1ncnc2c1ccn2[C@@H]1C[C@H](CO)[C@H]2OC(C)(C)O[C@H]21]

Compound 148 (2.93 g, 8.96 mmol) was dissolved in n-BuOH (22.40 mL). Then, a 33% MeNH₂ solution in EtOH (22.40 mL) was added. The mixture was stirred under microwave irradiation at 120° C. (100 W, 100 psi) for 25 min The volatiles were removed in vacuo and the resulting residue was purified over silica (CH₂Cl₂/MeOH; 0-10%) to afford the product as greyish foam (2.25 g, 79%). ¹H NMR (400 MHZ, DMSO-d₆) δ8.14 (s, 1H, H2), 7.46 (q, J=4.3 Hz, 1H, NH), 7.30 (d, J=3.5 Hz, 1H, H6), 6.56 (d, J=3.5 Hz, 1H, H5), 4.93 (dt, J=11.7, 6.3 Hz, 1H, H1′), 4.90-4.85 (m, 1H, H2′), 4.77 (t, J=5.4 Hz, 1H, 5′OH), 4.52 (dd, J=7.0, 4.5 Hz, 1H, H3′), 3.50 (td, J=5.4, 2.0 Hz, 2H, CH₂, H5′), 2.96 (d, J=4.7 Hz, 3H, CH₃, NMe), 2.23-2.13 (m, 2H, H4′, CH₂, cyclopentane), 2.08-1.99 (m, 1H, CH₂, cyclopentane), 1.47 (s, 3H, CH₃, acetonide), 1.22 (s, 3H, CH₃, acetonide).

### 7-((3αS,4R,6R,6αR)-6-(azidomethyl)-2,2-dimethyltetrahydro-4H-cyclopenta[d][1,3]dioxol-4-yl)-N-methyl-7H-pyrrolo[2,3-d]pyrimidin-4-amine (150)

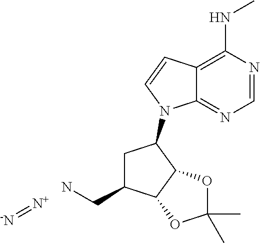

Compound 149 (1.30 g, 4.04 mmol) was dissolved in dry 1,4 dioxane (16.20 mL). The solution was cooled down to 0° C. Then, DPPA (1.76 mL, 8.09 mmol) and DBU (1.83 mL, 12.13 mmol) were added to the cooled solution. The reaction mixture was stirred magnetically overnight at rt. TLC monitored full consumption of the SM. Then, NaN₃ (1.33 g, 20.21 mmol) and 15-crown-5 (0.84 mL, 4.04 mmol) were added, and the mixture was heated to 110° C. At this temperature, the mixture was stirred for 6 h. After this time, the mixture was allowed to cool down to rt and the organic solvent was evaporated under vacuum. Afterwards, water was added, and the aqueous phase was extracted with EtOAc. The combined organic layers were washed with brine, dired over sodium sulfate, and concentrated under reduced pressure. The obtained residue was purified over silica (cyclohexane/EtOAc; 20-80%) afford the product as yellowish foam (0.61 g, 44%). ¹H NMR (400 MHZ, DMSO-d₆) δ8.15 (s, 1H, H2), 7.48 (q, J=4.5 Hz, 1H, NH), 7.30 (d, J=3.6 Hz, 1H, H6), 6.57 (d, J=3.5 Hz, 1H, H5), 4.96 (dt, J=11.7, 6.3 Hz, 1H, H1′), 4.95-4.86 (m, 1H, H2′), 4.52 (dd, J=7.1, 5.4 Hz, 1H, H3′), 3.58-3.45 (m, 2H, CH₂, H5′), 2.96 (d, J=4.7 Hz, 3H, CH₃, NMe), 2.36-2.21 (m, 2H, H4′, CH₂, cyclopentane), 2.12-2.01 (m, 1H, CH₂, cyclopentane), 1.48 (s, 3H, CH₃, acetonide), 1.23 (s, 3H, CH₃, acetonide).

### tert-butyl (7-((3αS,4R,6R,6αR)-6-(azidomethyl)-2,2-dimethyltetrahydro-4H-cyclopenta[d][1,3]dioxol-4-yl)-7H-pyrrolo[2,3-d]pyrimidin-4-yl)(methyl)carbamate (151)

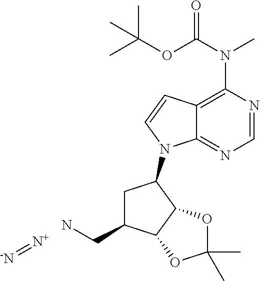

The azido-nucleoside 150 (0.60 g, 1.73 mmol) was dissolved in dry THF (7.50 mL) and cooled down in an ice-bath. To this cooled solution, NaH (60% in mineral oil, 0.14 g, 3.46 mmol) was added portion wise. The mixture was allowed to warm up rt and stirred for 30 min before the mixture was cooled down again to 0° C. Then, di-tert-butyl decarbonate (0.40 mL, 1.73 mmol) was added portion wise at 0° C. The mixture was stirred magnetically at rt for 6 h. TLC indicated slow conversion. At this point, the mixture was cooled down and 8 eq. NaH was added. The reaction mixture was stirred for 30 min at rt before the mixture was again cooled down to 0° C. Then, 3 eq. di-tert-butyl decarbonate was added and the mixture was stirred for 72 h at rt. At this point, TLC monitored still SM. Therefore, the mixture was cooled down and 5 eq NaH was added and the mixture was stirred at rt for 30 min. Then, the mixture was cooled down again and 2 eq. di-tert-butyl decarbonate were added. The reaction mixture was stirred for further 48 h at ambient temperature. After this time almost all SM was converted. Then, the mixture was cooled down and quenched carefully with water (highly exothermic reaction). Then, the aqueous phase was extracted with CH₂Cl₂. The combined organic layers were washed with brine, dried over sodium sulfate, and concentrated under vacuum. The crude product was purified by flash chromatography (CH₂Cl₂/MeOH, 0-10%) to afford the product as greyish foam (0.57 g, 74%). 1H NMR (400 MHZ, DMSO-d₆) δ 8.62 (s, 1H, H2), 7.72 (d, J=3.7 Hz, 1H, H6, 6.45 (d, J=3.7 Hz, 1H, H5), 5.12 (dt, J=12.1, 6.4 Hz, 1H, H1′), 4.92 (dd, J=7.2, 6.2 Hz, 1H, H2′), 4.53 (dd, J=7.3, 5.2 Hz, 1H, H3′), 3.53 (ddt, J=19.4, 12.4, 6.6 Hz, 2H, CH₂, H5′), 3.34 (s, 3H, CH₃, NMe), 2.40-2.26 (m, 2H, H4′, CH₂, cyclopentane), 2.16-2.06 (m, 1H, CH₂, cyclopentane), 1.49 (s, 3H, CH₃, acetonide), 1.44 (s, 9H, CH₃, t-butyl), 1.23 (s, 3H, CH₃, acetonide).

### 7-((3αS,4R,6R,6αR)-6-(azidomethyl)-2,2-dimethyltetrahydro-4H-cyclopenta[d][1,3]dioxol-4-yl)-N-methyl-7H-pyrrolo[2,3-d]pyrimidin-4-amine (152)

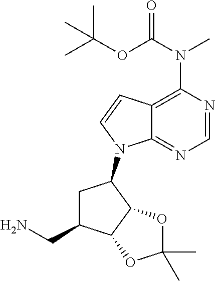 [SMILES: CN(C(=O)OC(C)(C)C)c1ncnc2c1ccn2[C@@H]1C[C@H](CN)[C@H]2OC(C)(C)O[C@H]21]

Compound 151 (0.56 g, 1.25 mmol) was dissolved in a mixture of EtOAc/MeOH (4.20 mL, 1:1) under nitrogen atmosphere. Then, the solution was degassed before palladium on activated charcoal moistened with water (0.10 g, 0.13 mmol) was added. The suspension was degassed and purged with H2 from a storage vessel. The reaction mixture was stirred for 17 h at ambient temperature. Then, the mixture was purged with nitrogen and filtered over celite. The filter cake was rinsed with MeOH and the filtrate was concentrated under reduced pressure. The obtained residue was purified over silica (CH₂Cl₂/MeOH, 0-20%) for afford the desired product as greyish foam (0.25 g, 48%). ¹H NMR (400 MHZ, DMSO-d₆) δ8.63 (s, 1H, H2), 7.76 (d, J=3.7 Hz, 1H, H6), 6.45 (d, J=3.7 Hz, 1H, H5), 5.09 (dt, J=12.1, 6.7 Hz, 1H, H1′), 4.91-4.86 (m, 1H, H2′), 4.51 (dd, J=7.2, 4.9 Hz, 1H, H3′), 3.35 (s, 3H, CH₃, NMe), 2.78-2.62 (m, 2H, CH₂, H5′), 2.31-2.23 (m, 1H, CH₂, cyclopentane), 2.19-2.08 (m, 1H, H4′), 2.08-1.99 (m, 1H, CH₂, cyclopentane), 1.48 (s, 3H, CH₃, acetonide), 1.45 (s, 9H, CH₃, t-butyl), 1.23 (s, 3H, CH₃, acetonide).

### Preparation of tert-butyl (7-((3αR,4R,6R,6αR)-6-(aminomethyl)-2,2-dimethyltetrahydrofuro[3,4-d][1,3]dioxol-4-yl)-5-methyl-7H-pyrrolo[2,3-d]pyrimidin-4-yl)(methyl)carbamate

### (2R,3R,4R,5R)-2-((benzoyloxy) methyl)-5-(4-chloro-5-methyl-7H-pyrrolo[2,3-d]pyrimidin-7-yl) tetrahydrofuran-3,4-diyl dibenzoate (153)

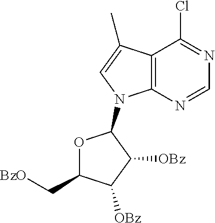

In a heat-dried three-necked round bottom flask was added 4-chloro-5-methyl-7H-pyrrolo[2,3-d]pyrimidine (2.32 g, 13.85 mmol) in dry MeCN (69.20 mL) under nitrogen. Then, BSA (3.76 mL, 15.23 mmol) was added to the stirred suspension. The suspension was stirred for 10 minutes. During this time the reaction became a homogenous solution. Then, the 1-O-Acetyl-2,3,5-tri-O-benzoyl-B-D-ribofuranose (7.76 g, 15.23 mmol) was added and the reaction mixture was heated to 85° C. for 5 h. After this the reaction was cooled down to rt before diluted with EtOAc (100 ml). The organic phase was washed with saturated bicarbonate solution (1×100 mL) and with brine (1×100 mL). The combined organic layers were dried over sodium sulfate, filtered, and concentrated under reduced pressure. The crude product was purified over silica (cyclohexane/EtOAc; 0-40%) to afford the product as yellowish foam. (2.60 g, 31%). ¹H NMR (400 MHz, DMSO-d₆) δ8.52 (s, 1H, H2), 8.03-7.89 (m, 6H, o-Ar, Bz), 7.73 (s, 1H, H6), 7.70-7.58 (m, 3H, p-Ar, Bz), 7.57-7.45 (m, 6H, m-Ar, Bz), 6.68 (d, J=5.2 Hz, 1H, H1′), 6.29 (t, J=6.0 Hz, 1H, H2′), 6.11 (t, J=5.6 Hz, 1H, H3′), 4.84 (q, J=3.6, 3.0 Hz, 1H, H4′), 4.79 (dd, J=12.0, 3.5 Hz, 1H, H5′), 4.64 (dd, J=12.0, 4.6 Hz, 1H, H6′), 2.34 (s, 3H, CH₃, Me).

### (2R,3S,4R,5R)-2-(hydroxymethyl)-5-(5-methyl-4-(methylamino)-7H-pyrrolo[2,3-d]pyrimidin-7-yl) tetrahydrofuran-3,4-diol (154)

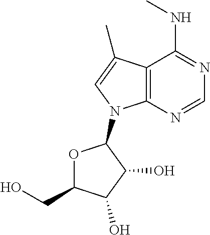 [SMILES: CNc1ncnc2c1c(C)cn2[C@@H]1O[C@H](CO)[C@@H](O)[C@H]1O]

The nucleoside 153 (4.18 g, 6.76 mmol) was dissolved in 1,4-dioxane (33.80 mL) and 33% MeNH₂ solution in EtOH (33.80 mL) in a sealed flask under nitrogen. The solution was heated to 60° C. overnight. Then, the reaction was allowed to cool down to rt before concentrated under reduced pressure. The obtained oil was purified by flash chromatography (CH₂Cl₂/MeOH; 0-20%) which afforded the product as slightly yellowish foam (1.69 g, 85%). ¹H NMR (400 MHZ, DMSO-d₆) δ8.11 (s, 1H, H2), 7.08 (s, 1H, H6), 6.56 (q, J=4.2 Hz, 1H, NH), 5.98 (d, J=6.4 Hz, 1H, H1′), 4.35 (t, J=5.7 Hz, 1H, H2′), 4.06 (dd, J=5.0, 3.2 Hz, 1H, H3′), 3.86 (q, J=3.7 Hz, 1H, H4′), 3.55 (ddd, J=34.8, 11.7, 3.6 Hz, 1H, H5′), 2.95 (d, J=4.6 Hz, 3H, CH₃, N-Me), 2.36 (s, 3H, CH₃, Me).

### ((3αR,4R,6R,6αR)-2,2-dimethyl-6-(5-methyl-4-(methylamino)-7H-pyrrolo[2,3-d]pyrimidin-7-yl) tetrahydrofuro[3,4-d][1,3]dioxol-4-yl) methanol (155)

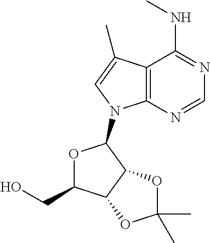 [SMILES: CNc1ncnc2c1c(C)cn2[C@@H]1O[C@H](CO)[C@H]2OC(C)(C)O[C@H]21]

Compound 154 (1.68 g, 5.65 mmol) was suspended in acetone (28.30 mL). To the stirred suspension triethoxymethane (4.76 mL, 28.27 mmol) and p-TsOH (5.43 g, 28.27 mmol) were added sequentially at rt. The suspension became a clear yellow solution and was stirred overnight at rt. Then, the mixture was quenched with 5% aqueous sodium bicarbonate solution. The organic solvent was removed under reduced pressure. The aqueous phase was extracted with CH₂Cl₂. The combined organic layers were washed with brine, dried over sodium sulfate, and concentrated over vacuum. The obtained residue was purified by flash chromatography (CH₂Cl₂/MeOH; 0-5%) which afforded the product as a colorless foam (1.38 g, 73%). ¹H NMR (400 MHZ, DMSO-d₆) δ8.12 (s, 1H, H2), 7.09 (d, J=1.1 Hz, 1H, H6), 6.60 (q, J=4.4 Hz, 1H, NH), 6.13 (d, J=3.7 Hz, 1H, H1′), 5.17 (t, J=5.6 Hz, 1H, OH), 5.09 (dd, J=6.3, 3.7 Hz, 1H, H2′), 4.87 (dd, J=6.4, 3.0 Hz, 1H, H3′), 4.07 (td, J=4.7, 3.2 Hz, 1H, H4′), 3.58-3.45 (m, 2H, CH₂, H5′), 2.94 (d, J=4.6 Hz, 3H, CH₃, NMe), 2.35 (d, J=1.0 Hz, 3H, CH₃), 1.53 (s, 3H, CH₃, acetonide), 1.30 (s, 3H, CH₃, acetonide).

### 7-((3αR,4R,6R,6αR)-6-(azidomethyl)-2,2-dimethyltetrahydrofuro[3,4-d][1,3]dioxol-4-yl)-N,5-dimethyl-7H-pyrrolo[2,3-d]pyrimidin-4-amine (156)

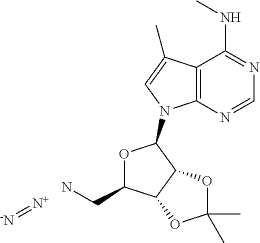

Compound 155 (1.37 g, 4.05 mmol) was dissolved in dry 1,4-dioxane (20.30 mL) and then cooled down in an ice-bath. Afterwards, DPPA (1.76 mL, 8.11 mmol) and DBU (1.83 mL, 12.16 mmol) were added to the cooled solution and stirred for 24 h at rt. Then, 15-crown-5 (0.85 mL, 4.05 mmol) and NaN₃ (1.33 g, 20.27 mmol) were added and the suspension was heated to 110° C. After 7 h, the reaction mixture was allowed to cool down to rt and then the organic solvent was evaporated. The obtained oil was diluted with water and the aqueous phase was extracted three times with EtOAc. The combined organic layers were washed with brine, dried over sodium sulfate, and concentrated under reduced pressure. The crude product was purified over silica ( ) to obtain the desired product as colorless foam (0.71 g, 49%). ¹H NMR (400 MHZ, DMSO-d₆) δ 8.15 (s, 1H, H2), 7.12-7.07 (m, 1H, H6), 6.62 (q, J=4.3 Hz, 1H, NH), 6.20 (d, J=3.2 Hz, 1H, H1′), 5.25 (dd, J=6.4, 3.2 Hz, 1H, H2′), 4.91 (dd, J=6.4, 3.5 Hz, 1H, H3′), 4.22-4.15 (m, 1H, H4′), 3.61-3.49 (m, 2H, CH₂, H5′), 2.95 (d, J=4.6 Hz, 3H, CH₃, NMe), 2.39-2.34 (m, 3H, CH₃), 1.53 (s, 3H, CH₃, acetonide), 1.32 (s, 3H, CH₃, acetonide).

### tert-butyl (7-((3αR,4R,6R,6αR)-6-(azidomethyl)-2,2-dimethyltetrahydrofuro[3,4-d][1,3]dioxol-4-yl)-5-methyl-7H-pyrrolo[2,3-d]pyrimidin-4-yl)(methyl)carbamate (157)

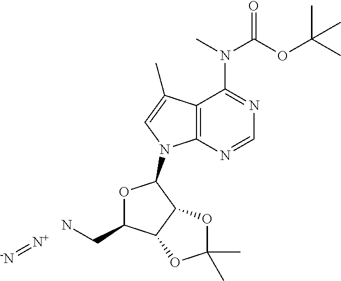

Compound 156 (0.69 g, 1.91 mmol) was dissolved in dry THF (9.60 mL) under nitrogen atmosphere. To the solution, triethylamine (0.40 mL, 2.89 mmol) and DMAP (0.12 g, 0.96 mmol) were added at rt. Then, the solution was cooled down in an ice-bath before di-tert butyl dicarbonate (0.67 mL, 2.87 mmol) was added portion wise. The reaction mixture was stirred overnight at ambient temperature. TLC(cyclohexane/EtOAc; 60%) indicated almost complete conversion. After 18 h the reaction mixture was diluted with saturated sodium bicarbonate solution and extracted with CH₂Cl₂. The combined organic layers were washed with brine, dried over sodium sulfate, and concentrated under reduced pressure. The crude product was purified by flash chromatography (cyclohexane/EtOAc; 0-80%) to afford the product as greyish foam (0.66 g, 75%). ¹H NMR (400 MHZ, DMSO-d₆) δ8.69 (s, 1H, 1H, H2), 7.60-7.57 (m, 1H, H6), 6.34 (d, J=3.2 Hz, 1H, H1′), 5.32 (dd, J=6.4, 3.2 Hz, 1H, H2′), 4.96 (dd, J=6.4, 3.5 Hz, 1H, H3′), 4.26 (dt, J=6.3, 4.5 Hz, 1H, H4′), 3.64-3.51 (m, 2H, CH₂, H5′), 3.26 (s, 3H, CH₃, NMe), 2.24 (d, J=1.0 Hz, 3H, CH₃), 1.56 (s, 3H, CH₃, acetonide), 1.38 (s, 9H, CH₃, t-butyl), 1.34 (s, 3H, CH₃, acetonide).

### tert-butyl (7-((3αR,4R,6R,6αR)-6-(aminomethyl)-2,2-dimethyltetrahydrofuro[3,4-d][1,3]dioxol-4-yl)-5-methyl-7H-pyrrolo[2,3-d]pyrimidin-4-yl)(methyl)carbamate (158)

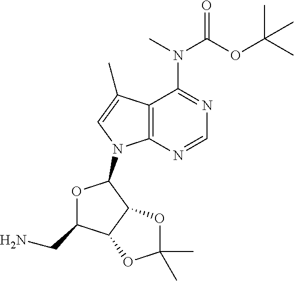 [SMILES: Cc1cn([C@@H]2O[C@H](CN)[C@H]3OC(C)(C)O[C@H]32)c2ncnc(N(C)C(=O)OC(C)(C)C)c12]

The azido nucleoside 157 (0.65 g, 1.40 mmol) was dissolved in EtOH (14.00 mL) under nitrogen atmosphere. The solution was degassed and then palladium on activated charcoal moistened with water (0.30 g, 0.14 mmol) was added under nitrogen. The reaction mixture was purged with hydrogen from a storage vessel (1 atm) and stirred at ambient temperature overnight. TLC (CH₂Cl₂/MeOH; 10%) indicated full conversion. Then, the mixture was purged with nitrogen and filtered over Celite. The filter cake was rinsed with EtOH and CH₂Cl₂. The filtrated was concentrated under reduced pressure. The obtained residue was purified over silica (CH₂Cl₂/MeOH; 20%) to afford the product as greyish foam (0.53 g, 87%). ¹H NMR (400 MHZ, DMSO-d₆) δ8.67 (s, 1H, H2), 7.60 (d, J=1.1 Hz, 1H, H6), 6.27 (d, J=3.7 Hz, 1H, H1′), 5.23 (dd, J=6.5, 3.7 Hz, 1H, H2′), 4.94 (dd, J=6.5, 3.2 Hz, 1H, H3′), 4.04 (td, J=5.6, 3.2 Hz, 1H, H4′), 3.26 (s, 3H, CH₃, N-Me), 2.72 (dd, J=5.6, 3.9 Hz, 2H H5′), 2.24 (d, J=1.1 Hz, 3H, CH₃, 7′Me), 1.57 (bs, 2H, NH₂), 1.55 (s, 3H, CH₃, acetonide), 1.39 (s, 9H, CH₃, t-butyl), 1.33 (s, 3H, CH₃, acetonide).

### 7-((3αS,4R,6R,6αR)-6-(aminomethyl)-2,2-dimethyltetrahydro-4H-cyclopenta[d][1,3]dioxol-4-yl)-5-bromo-N-(4-methoxybenzyl)-7H-pyrrolo[2,3-d]pyrimidin-4-amine (1R,2S,3R,5R)-3-(4-chloro-7H-pyrrolo[2,3-d]pyrimidin-7-yl)-5-(hydroxymethyl) cyclopentane-1,2-diol (159)

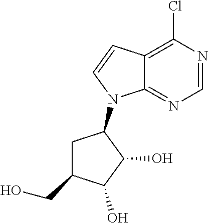 [SMILES: OC[C@H]1C[C@@H](n2ccc3c(Cl)ncnc32)[C@H](O)[C@@H]1O]

The 3-amino-5-(hydroxymethyl) cyclopentane-1,2-diol (5.00 g, 26.96 mmol) was dissolved in EtOH (234.40 mL). To this solution, 4,6-dichloropyrimidin-5-acetaldehyde (5.15 g, 26.96 mmol) and triethylamine (12.52 mL, 53.91 mmol) were added at ambient temperature. The reaction mixture was heated to 90° C. and stirred magnetically overnight. After 18 h, the volatiles were evaporated under reduces pressure and the obtained residue was purified over silica (CH₂Cl₂/MeOH; 0-10%) which afforded the product as yellowish solid (6.96 g, 91%). ¹H NMR (400 MHz, DMSO-d₆) δ8.63 (s, 1H, H2), 7.91 (d, J=3.7 Hz, 1H, H6), 6.70 (d, J=3.6 Hz, 1H, H5), 5.05 (dt, J=10.4, 8.6 Hz, 1H, H1′), 4.87 (d, J=6.7 Hz, 1H, 2′OH), 4.76 (t, J=5.2 Hz, 1H, 5′OH), 4.70 (d, J=4.2 Hz, 1H, 3′OH), 4.24 (ddd, J=9.2, 6.6, 5.3 Hz, 1H, H2′), 3.84 (dq, J=4.9, 3.0 Hz, 1H, H3′), 3.55-3.43 (m, 2H, CH₂, H5′), 2.23 (dt, J=12.8, 8.7 Hz, 1H, CH₂, cyclopentane), 2.11-2.01 (m, 1H, H4′), 1.61 (ddd, J=12.9, 10.5, 7.9 Hz, 1H, CH₂, cyclopentane).

### ((3αR,4R,6R,6αS)-6-(4-chloro-7H-pyrrolo[2,3-d]pyrimidin-7-yl)-2,2-dimethyltetrahydro-4H-cyclopenta[d][1.3]dioxol-4-yl) methanol (160)

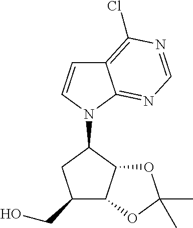 [SMILES: CC1(C)O[C@@H]2[C@@H](CO)C[C@@H](n3ccc4c(Cl)ncnc43)[C@@H]2O1]

To a suspension of compound 159 (4.21 g, 14.67 mmol) in acetone (391.30 mL), triethoxymethane (12.33 mL, 73.37 mmol) and pTsOH (14.10 g, 73.37 mmol) were added. The suspension became a clear solution and was stirred at ambient temperature for 20 h. Then, the reaction mixture was quenched with 5% aqueous sodium bicarbonate solution. The organic solvent was removed under reduced pressure. The aqueous phase was extracted with CH₂Cl₂. The combined organic layers were washed with brine, dried over sodium sulfate, and concentrated. The obtained residue was purified over silica (CH₂Cl₂/MeOH; 0-5%) to afford the product as yellowish foam (4.90 g, 62%). ¹H NMR (400 MHZ, DMSO-d₆) δ8.66 (s, 1H, H2), 7.97 (d, J=3.7 Hz, 1H, H6), 6.73 (d, J=3.7 Hz, 1H, H5), 5.10 (dt, J=13.0, 6.5 Hz, 1H, H1’), 4.92 (t, J=6.8 Hz, 1H, H2′), 4.82 (t, J=5.3 Hz, 1H, 5′OH), 4.55 (dd, J=7.2, 4.3 Hz, 1H, H3′), 3.53 (td, J=5.4, 1.3 Hz, 2H, CH₂, H5′), 2.31-2.20 (m, 1H, H4′, CH₂, cyclopentane), 2.14-2.09 (m, 1H, CH₂, cyclopentane), 1.49 (s, 3H, CH₃, acetonide), 1.23 (s, 3H, CH₃, acetonide).

### 7-((3αS,4R,6R,6αR)-6-(((tert-butyldimethylsilyl)oxy) methyl)-2,2-dimethyltetrahydro-4H-cyclopenta[d][1,3]dioxol-4-yl)-4-chloro-7H-pyrrolo[2,3-d]pyrimidine (161)

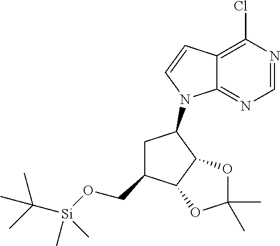 [SMILES: CC1(C)O[C@@H]2[C@@H](CO[Si](C)(C)C(C)(C)C)C[C@@H](n3ccc4c(Cl)ncnc43)[C@@H]2O1]

TBSCl (2.15 g, 14.15 mmol) was added to a solution of compound 160 (3.86 g, 11.79 mmol) and imidazole (1.95 g, 28.29 mmol) in CH₂Cl₂ (58.90 mL). The reaction mixture was stirred magnetically at ambient temperature overnight. After 18 h, the mixture was quenched with saturated bicarbonate solution and extracted with CH₂Cl₂. The combined organic layers were washed with brine, dried over sodium sulfate, and concentrated under reduced pressure. The crude product was purified by FC(cyclohexane/EtOAc; 0-100%) to afford the product as yellowish oil (4.79 g, 93%). ¹H NMR (400 MHZ, DMSO-d₆) δ8.63 (s, 1H, H2), 7.92 (d, J=3.7 Hz, 1H, H6), 6.73 (d, J=3.7 Hz, 1H, H5), 5.12-5.04 (m, 1H, H1′), 4.96-4.91 (m, 1H, H2′), 4.56-4.49 (m, 1H, H3′), 3.76-3.63 (m, 2H, CH₂, H5′), 2.31-2.21 (m, 2H, CH₂, cyclopentane & H4′), 2.21-2.12 (m, 1H, CH₂, cyclopentane), 1.48 (s, 3H, CH₃, acetonide), 1.21 (s, 3H, CH₃, acetonide), 0.88 (s, 9H, CH₃, t-butyl, OTBS), 0.06-0.05 (m, 6H, CH₃, Me, OTBS).

### 5-bromo-7-((3αS,4R,6R,6αR)-6-(((tert-butyldimethylsilyl)oxy) methyl)-2,2-dimethyltetrahydro-4H-cyclopenta[d][1,3]dioxol-4-yl)-4-chloro-7H-pyrrolo[2,3-d]pyrimidine (162)

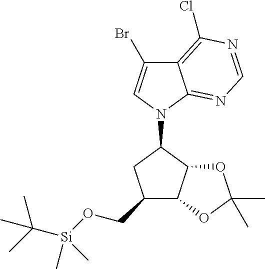 [SMILES: CC1(C)O[C@@H]2[C@@H](CO[Si](C)(C)C(C)(C)C)C[C@@H](n3cc(Br)c4c(Cl)ncnc43)[C@@H]2O1]

A solution of NBS (2.04 g, 11.32 mmol) in DMF (20.00 mL) was added dropwise onto a solution of compound 161 (4.77 g, 10.78 mmol) in DMF (33.90 mL). The reaction mixture was stirred at 35° C. for 30 min. The mixture was allowed to cool down to rt and then water was added. The mixture was extracted with EtOAc. The combined organic layers were dried over sodium sulfate, filtered, and concentrated in vacuo. The obtained residue was purified over silica (cyclohexane/EtOAc; 0-100%) to afford the product as yellowish solid (4.67 g, 84%). ¹H NMR (400 MHz, DMSO-d₆) δ8.67 (s, 1H, H2), 8.21 (s, 1H, H6), 5.10 (dt, J=12.5, 6.3 Hz, 1H, H1′), 4.92 (dd, J=7.1, 5.9 Hz, 1H, H2′), 4.50 (dd, J=7.1, 4.5 Hz, 1H, H3′), 3.74-3.62 (m, 2H, CH₂, H5′), 2.32-2.22 (m, 2H, CH₂, cyclopentane & H4′), 2.18-2.07 (m, 1H, CH₂, cyclopentane), 1.47 (s, 3H, CH₃, acetonide), 1.21 (s, 3H, CH₃, acetonide), 0.88 (s, 9H, CH₃, t-butyl, OTBS), 0.05 (s, 6H, CH₃, Me, OTBS).

### 5-bromo-7-((3αS,4R,6R,6αR)-6-(((tert-butyldimethylsilyl)oxy) methyl)-2,2-dimethyltetrahydro-4H-cyclopenta[d][1,3]dioxol-4-yl)-N-(4-methoxybenzyl)-7H-pyrrolo[2,3-d]pyrimidin-4-amine (163)

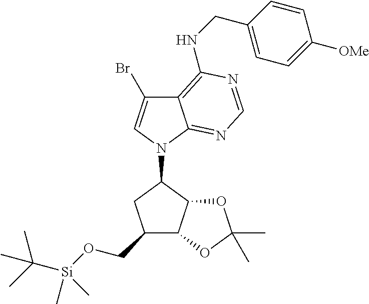 [SMILES: COc1ccc(CNc2ncnc3c2c(Br)cn3[C@@H]2C[C@H](CO[Si](C)(C)C(C)(C)C)[C@H]3OC(C)(C)O[C@H]32)cc1]

To a solution of compound 162 (4.65 g, 8.91 mmol) in 1,4-dioxane (44.50 mL), Et₃N (1.88 mL, 13.36 mmol) was added. Then, 4-methoxybenzylamine (1.41 mL, 10.69 mmol) was added portion wise and the resulted mixture was stirred at 80° C. over 2 d. The mixture was allowed to cool down to rt. Then, the organic solvent was evaporated in vacuo. To the obtained residue saturated bicarbonate solution was added and then extracted with EtOAc. The combined organic layers were washed with brine, dried over sodium sulfate, and concentrated in vacuo. The crude product was purified by FC(cyclohexane/EtOAc; 0-50%) to afford the desired product as yellowish solid (4.32 g, 79%). ¹H NMR (400 MHZ, DMSO-d₆) δ 8.15 (s, 1H, H2), 7.61 (s, 1H, H6), 7.31-7.25 (m, 2H, o-benzyl), 6.98 (t, J=6.1 Hz, 1H, NH), 6.90-6.83 (m, 2H, m-benzyl), 4.96 (dt, J=11.6, 6.2 Hz, 1H, H1′), 4.88 (dd, J=7.1, 5.9 Hz, 1H, H2′), 4.69 (d, J=6.1 Hz, 2H, CH₂, PMB), 4.47 (dd, J=7.0, 4.6 Hz, 1H, H3′), 3.71 (s, 3H, CH₃, OMe), 3.70-3.60 (m, 2H, CH₂, H5′), 2.29-2.14 (m, 2H, CH₂, cyclopentane & H4′), 2.10-2.01 (m, 1H, CH₂, cyclopentane), 1.45 (s, 3H, CH₃, acetonide), 1.20 (s, 3H, CH₃, acetonide), 0.87 (s, 9H, CH₃, t-butyl, OTBS), 0.05 (s, 6H, CH₃, Me, OTBS).

### ((3αR,4R,6R,6αS)-6-(5-bromo-4-((4-methoxybenzyl)amino)-7H-pyrrolo[2,3-d]pyrimidin-7-yl)-2,2-dimethyltetrahydro-4H-cyclopenta[d][1,3]dioxol-4-yl) methanol (164)

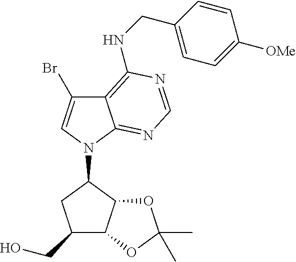 [SMILES: COc1ccc(CNc2ncnc3c2c(Br)cn3[C@@H]2C[C@H](CO)[C@H]3OC(C)(C)O[C@H]32)cc1]

Compound 163 (2.35 g, 3.77 mmol) was dissolved in dry THF (18.80 mL). Then, a 1 M TBAF (1.42 mL, 5.65 mmol) solution in THF (4.23 mL) was added dropwise. The reaction mixture was stirred at ambient temperature for 4 h. Then, the mixture was diluted with water and extracted with CH₂Cl₂. The combined organic layers were dried over sodium sulfate and concentrated in vacuo. The crude product was purified over silica (cyclohexane/EtOAc; 0-100%) to afford the product as yellowish foam (1.50 g, 79%). ¹H NMR (400 MHZ, DMSO-d₆) δ8.17 (s, 1H, H2), 7.65 (s, 1H, H6), 7.31-7.26 (m, 2H, o-benzyl), 6.97 (t, J=6.0 Hz, 1H, NH), 6.90-6.84 (m, 2H-m-benzyl), 4.96 (dt, J=12.6, 6.4 Hz, 1H, H1′), 4.85 (t, J=6.7 Hz, 1H, H2′), 4.76 (t, J=5.3 Hz, 1H, 5′OH), 4.69 (d, J=6.1 Hz, 2H, CH₂, PMB), 4.49 (dd, J=7.2, 4.4 Hz, 1H, H3′), 3.71 (s, 3H, CH₃. OMe), 3.52-3.45 (m, 2H, CH₂, H5′), 2.22-2.13 (m, CH₂, cyclopentane & H4′), 2.02-1.96 (m, 1H, CH₂, cyclopentane), 1.45 (s, 3H, CH₃, acetonide), 1.21 (s, 3H, CH₃, acetonide).

### 2-(((3αR,4R,6R,6αS)-6-(5-bromo-4-((4-methoxybenzyl)amino)-7H-pyrrolo[2,3-d]pyrimidin-7-yl)-2,2-dimethyltetrahydro-4H-cyclopenta[d][1,3]dioxol-4-yl)methyl) isoindoline-1,3-dione (165)

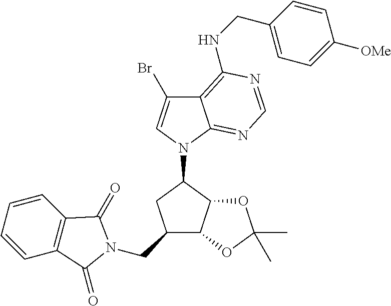 [SMILES: COc1ccc(CNc2ncnc3c2c(Br)cn3[C@@H]2C[C@H](CN3C(=O)c4ccccc4C3=O)[C@H]3OC(C)(C)O[C@H]32)cc1]

To a solution of the alcohol 164 (1.52 g, 2.99 mmol) in dry THF (29.90 mL), phthalimide (0.58 g, 3.89 mmol), PPh₃ (1.03 g, 3.89 mmol), and DEAD (0.62 mL, 3.89 mmol) were added in this order. The solution was stirred overnight at ambient temperature. The reaction mixture was diluted with water and was extracted with CH₂Cl₂. The combined organic layers were washed with brine, dried over sodium sulfate, and concentrated in vacuo. The crude product was purified by FC (cyclohexane/EtOAc; 0-100%) to afford the product as colorless foam (1.33 g, 70%). ¹H NMR (400 MHZ, DMSO-d₆) δ8.12 (s, 1H, H2), 7.90-7.87 (m, 2H, H5/6, phthalimide), 7.85-7.82 (m, 2H, H4/7, phthalimide), 7.71 (s, 1H, H6), 7.29-7.25 (m, 2H, o-benzyl), 6.97 (t, J=6.0 Hz, 1H, NH), 6.89-6.84 (m, 2H, m-benzyl), 5.02-4.93 (m, 1H, H1′), 4.82 (dd, J=7.4, 5.3 Hz, 1H, H2′), 4.69 (d, J=6.1 Hz, 2H, CH₂, PMB), 4.59-4.53 (m, 1H, H3′), 3.85-3.65 (m, 2H, CH₂, H5′), 3.71 (s, 3H, CH₃, OMe), 2.47-2.40 (m, 1H, H4′), 2.25 (dt, J=13.0, 6.7 Hz, 1H, CH₂, cyclopentane), 2.14-2.02 (m, 1H, CH₂, cyclopentane), 1.38 (s, 3H, CH₃, acetonide), 1.14 (s, 3H, CH₃, acetonide).

### 7-((3αS,4R,6R,6αR)-6-(aminomethyl)-2,2-dimethyltetrahydro-4H-cyclopenta[d][1,3]dioxol-4-yl)-5-bromo-N-(4-methoxybenzyl)-7H-pyrrolo[2,3-d]pyrimidin-4-amine (166)

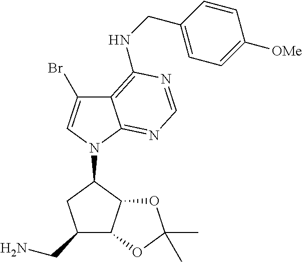 [SMILES: COc1ccc(CNc2ncnc3c2c(Br)cn3[C@@H]2C[C@H](CN)[C@H]3OC(C)(C)O[C@H]32)cc1]

Hydrazine monohydrate (1.62 mL, 33.06 mmol) was added to a suspension of compound 165 (1.32 g, 2.07 mmol) in EtOH (51.70 mL) and stirred at reflux for 1 h. After 10 min the suspension became a clear solution. After 20 min, a precipitation is formed. The reaction mixture was cooled down to 0° C. in an ice-bath. The suspension was filtered, and the filter cake was washed with EtOH. The filtrate was evaporated in vacuo and the obtained residue was purified by FC (CH₂Cl₂/MeOH, 0-10%) to afford the product as colorless foam (0.79 g, 76%). ¹H NMR (400 MHZ, DMSO-d₆) δ8.17 (s, 1H, H2), 7.67 (s, 1H, H6), 7.31-7.26 (m, 2H, o benzyl), 6.97 (t, J=6.0 Hz, 1H, NH), 6.90-6.84 (m, 2H, m-benzyl), 4.96 (dt, J=11.7, 6.6 Hz, 1H, H1′), 4.85-4.80 (m, 1H, H2′), 4.69 (d, J=6.1 Hz, 2H, CH₂, PMB), 4.45 (dd, J=7.2, 5.0 Hz, 1H, H3′), 3.71 (s, 3H, CH₃, OMe), 2.74-2.57 (m, 2H, CH₂, H5′), 2.21 (dt, J=12.2, 6.6 Hz, CH₂, cyclopentane), 2.14-2.01 (m, 1H, H4′), 2.00-1.92 (m, 1H, CH₂, cyclopentane), 1.45 (s, 3H, CH₃, acetonide), 1.21 (s, 3H, CH₃, acetonide).

### Synthesis of 7-((3as,4r,6r,6ar)-6-(aminomethyl)-2,2-dimethyltetrahydro-4H-cyclopenta[d][1,3]dioxol-4-yl)-N-(4-methoxybenzyl)-5-phenyl-7H-pyrrolo[2,3-d]pyrimidin-4-amine

### 7-((3as,4r,6r,6ar)-6-(((tert-butyldimethylsilyl)oxy) methyl)-2,2-dimethyltetrahydro-4H-cyclopenta[d][1,3]dioxol-4-yl)-4-chloro-5-phenyl-7H-pyrrolo[2,3-d]pyrimidine (167)

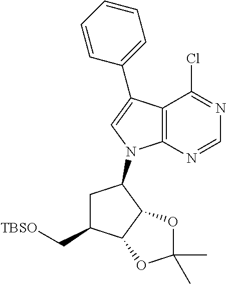 [SMILES: CC1(C)O[C@@H]2[C@@H](CO[Si](C)(C)C(C)(C)C)C[C@@H](n3cc(-c4ccccc4)c4c(Cl)ncnc43)[C@@H]2O1]

1228 mg of 162 (2.38 mmol; 1.0 eq) were dissolved in 25 ml degassed H₂O and 25 mL dioxane. 319 mg (2.62 mmol; 1.1 eq)phenylboronic acid, 1010 mg (4.76 mmol; 2.0 eq) K₃PO4 and 156 mg (0.24 mmol; 0.1 eq) Pd (dtbpf) Cl₂ were added. The reaction solution was heated at 80° C. for 15 h under N₂ atmosphere. The dioxan was removed under reduced pressure and the residue obtained was mixed with 60 mL EtOAc and extracted 3 times. After column chromatography on silica gel (cyclohexane/EtOAc 100:0% to 70:30%), 490 mg (0.95 mmol; 40%) of the title compound was obtained as a colorless oil. 7-((3as,4r,6r,6ar)-6-(((tert-butyldimethylsilyl)oxy) methyl)-2,2-dimethyltetrahydro-4H-cyclopenta[d][1,3]dioxol-4-yl)-N-(4-methoxybenzyl)-5-phenyl-7H-pyrrolo[2,3-d]pyrimidin-4-amine (168)

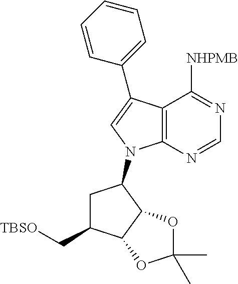

490 mg of 16/(0.95 mmol; 1.0 eq) 7-((3as,4r,6r,6ar)-6-(((tert-butyldimethylsilyl)oxy) methyl)-2,2-dimethyltetrahydro-4H-cyclopenta[d][1,3]dioxol-4-yl)-4-chloro-5-phenyl-7H-pyrrolo[2,3-d]pyrimidine were dissolved in 15 mL dioxane and mixed with 0.20 ml (1.43 mmol; 1.5 eq) Et₃N. To this were added 0.15 ml (1.14 mmol; 1.2 eq) of 4-methoxybenzylamine, and the reaction solution was heated at reflux for 72 h. Then the solvent was removed under reduced pressure. After purification by column chromatography on silica gel (cyclohexane/EtOAc 100:0% to 70:30%), 370 mg (0.60 mmol; 63%) of the title compound was obtained as a colorless foam.

### ((3αR,4R,6r,6as)-6-(4-((4-methoxybenzyl)amino)-5-phenyl-7H-pyrrolo[2,3-d]pyrimidin-7-yl)-2,2-dimethyltetrahydro-4H-cyclopenta[d][1,3]dioxol-4-yl) methanol (169)

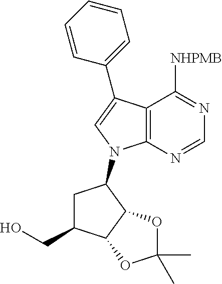

370 mg of 168 (0.60 mmol; 1.0 eq) 7-((3as,4r,6r,6ar)-6-(((tert-butyldimethylsilyl)oxy) methyl)-2,2-dimethyltetrahydro-4H-cyclopenta[d][1,3]dioxol-4-yl)-N-(4-methoxybenzyl)-5-phenyl-7H-pyrrolo[2,3-d]pyrimidin-4-amine were dissolved in 12 mL THF and reacted with 0.90 mL (0.90 mmol; 1.5 eq) of a 1 m solution of TBAF in THF. After 18 h, the reaction was stopped by addition of 20 mL dist. H₂O and extracted with DCM (3×60 mL). The combined organic phases were dried over MgSO₄, filtered, and the solvent was removed under reduced pressure. Column chromatography on silica gel (cyclohexane/EtOAc 100:0% to 50:50%) gave 275 mg (0.55 mmol; 92%) of the title compound in the form of a colorless foam.

### 7-((3as,4r,6r,6ar)-6-(aminomethyl)-2,2-dimethyltetrahydro-4//-cyclopenta[d][1,3]dioxol-4-yl)-N-(4-methoxybenzyl)-5-phenyl-7H-pyrrolo[2,3-d]pyrimidin-4-amine (170)

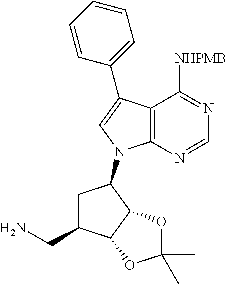

275 mg of 169 (0.55 mmol; 1.0 eq)((3αR,4R,6r,6as)-6-(4-((4-methoxybenzyl)amino)-5-phenyl-7 H-pyrrolo[2,3-d]pyrimidin-7-yl)-2,2-dimethyltetrahydro-4H-cyclopenta[d][1,3]dioxol-4-yl) methanol were dissolved in 10 mL THF. There were first added 105 mg (0.72 mmol; 1.3 eq) phthalimide, then 188 mg (0.72 mmol; 1.3 eq) PPh₃ and finally 0.31 mL (0.72 mmol; 1.3 eq) of a 40% solution of azodicarboxylic acid diethyl ester in toluene. The reaction solution was stirred at RT for 16 h and afterwards adsorbed on silica gel. The residue was roughly cleaned by column chromatography (cyclohexane/EtOAc 100:0% to 50:50%) and then after drying used without further purification. The residue was therefore dissolved in 25 ml EtOH. 0.43 ml (8.80 mmol; 16 eq) hydrazine hydrate were added and the resulting solution was heated under refluxation for 3 h. The reaction mixture was cooled to 0° C. The resulting precipitate was filtered off and the residue was washed with 80 mL EtOH. Column chromatography on silica gel (DCM/MeOH 100:0% to 90:10%) gave 275 mg (0.55 mmol; quantitative over 2 steps; crude) of the title compound in the form of a colorless foam.

### Synthesis of 7-((3as,4r,6r,6ar)-6-(aminomethyl)-2,2-dimethyltetrahydro-4H-cyclopenta[d][1,3]dioxol-4-yl)-5-iodo-N-(4-methoxybenzyl)-7H-pyrrolo[2,3-d]pyrimidin-4-amine

### 7-((3as,4R,6R,6ar)-6-(((tert-butyldimethylsilyl)oxy) methyl)-2,2-dimethyltetrahydro-4H-cyclopenta[d][1,3]dioxol-4-yl)-4-chloro-5-iodo-7H-pyrrolo[2,3-d]pyrimidine (171)

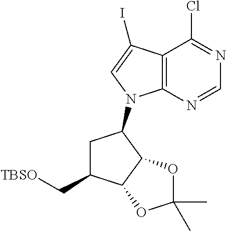 [SMILES: CC1(C)O[C@@H]2[C@@H](CO[Si](C)(C)C(C)(C)C)C[C@@H](n3cc(I)c4c(Cl)ncnc43)[C@@H]2O1]

1348 mg of 7-((3αS,4R,6R,6αR)-6-(((tert-butyldimethylsilyl)oxy) methyl)-2,2-dimethyltetrahydro-4H-cyclopenta[d][1,3]dioxol-4-yl)-4-chloro-7H-pyrrolo[2,3-a]pyrimidine (3.08 mmol; 1.0 eq) were dissolved in 20 mL DMF and 762 mg (3.39 mmol; 1.1 eq) of N-iodosuccinimide were added. The reaction solution was heated to 60° C. for 15 h. The solvent was removed under reduced pressure and the resulting residue was taken up in 120 mL EtOAc. The organic phase was washed with a saturated NaCl solution, dried over MgSO₄, filtered, and adsorbed on silica gel. Column chromatography (cyclohexane/EtOAc 100:0% to 90:10%) gave 1200 mg (2.13 mmol; 69%) of the title compound in the form of a colorless foam.

### 7-((3as,4r,6r,6ar)-6-(((tert-butyldimethylsilyl)oxy) methyl)-2,2-dimethyltetrahydro-4H-cyclopenta[d][1,3]dioxol-4-yl)-5-iodo-N-(4-methoxybenzyl)-7H-pyrrolo[2,3-d]pyrimidin-4-amine (172)

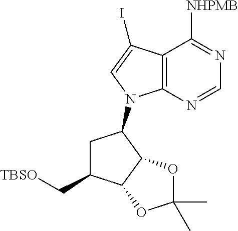

1200 mg of 171 (2.13 mmol; 1.0 eq) were dissolved in 25 mL dioxane and mixed with 0.44 mL (3.19 mmol; 1.5 eq) Et₃N. To this were added 0.33 mL (2.55 mmol; 1.2 eq) of 4-methoxybenzylamine, and the reaction solution was heated at reflux for 48 h. Then the solvent was removed under reduced pressure. After purification by column chromatography on silica gel (cyclohexane/EtOAc 100:0% to 60:40%), 1090 mg (1.64 mmol; 77%) of the title compound was obtained as a colorless foam.

### ((3αR,4R,6r,6as)-6-(5-iodo-4-((4-methoxybenzyl)amino)-7//pyrrolo[2,3-d]pyrimidin-7-yl)-2,2-dimethyltetrahydro-4H-cyclopenta[d][1,3]dioxol-4-yl) methanol (173)

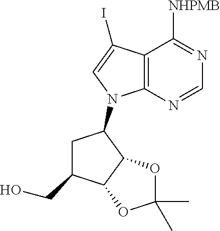

1090 mg of 172 (1.64 mmol; 1.0 eq) were dissolved in 15 mL THF and reacted with 2.46 mL (2.46 mmol; 1.5 eq) of a 1 m solution of TBAF in THF. After 16 h, the reaction was stopped by addition of 30 mL dist. H₂O and extracted with DCM (3×80 mL). The combined organic phases were dried over MgSO₄, filtered, and the solvent was removed under reduced pressure. Column chromatography on silica gel (cyclohexane/EtOAc 100:0% to 20:80%) gave 854 mg (1.55 mmol; 95%) of the title compound in the form of a colorless foam.

### 7-((3as,4r,6r,6ar)-6-(aminomethyl)-2,2-dimethyltetrahydro-4H-cyclopenta[d][1,3]dioxol-4-yl)-5-iodo-N-(4-methoxybenzyl)-7H-pyrrolo[2,3-d]pyrimidin-4-amine (174)

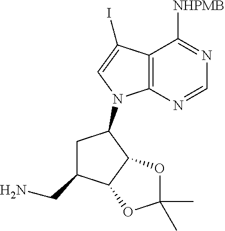

854 mg of 173 (1.55 mmol; 1.0 eq) were dissolved in 20 mL THF. There were first added 296 mg (2.02 mmol; 1.3 eq) phthalimide, then 529 mg (2.02 mmol; 1.3 eq) PPh₃ and finally 0.79 mL (2.02 mmol; 1.3 eq) of a 40% solution of azodicarboxylic acid diethyl ester in toluene. The reaction solution was stirred at RT for 18 h and afterwards adsorbed on silica gel. The residue was roughly cleaned by column chromatography (cyclohexane/EtOAc 100:0% to 50:50%) and then after drying used without further purification. The residue was therefore dissolved in 40 ml EtOH. 1.20 mL (24.80 mmol; 16 eq) hydrazine hydrate were added and the resulting solution was heated under refluxation for 2 h. The reaction mixture was cooled to 0° C. The resulting precipitate was filtered off and the residue was washed with 80 mL EtOH. Column chromatography on silica gel (DCM/MeOH 100:0% to 80:10%) gave 600 mg (1.09 mmol; 70% over 2 steps) of the title compound in the form of a colorless foam.

### Preparation of tert-butyl (3-aminopropyl)(((3αR,4R,6R,6αS)-6-(5-bromo-4-((4-methoxybenzyl)amino)-7H-pyrrolo[2,3-d]pyrimidin-7-yl)-2,2-dimethyltetrahydro-4H-cyclopenta[d][1,3]dioxol-4-yl)methyl)carbamate

### (9H-fluoren-9-yl) methyl(3-((((3αR,4R,6R,6αS)-6-(5-bromo-4-((4-methoxybenzyl)amino)-7H-pyrrolo[2,3-d]pyrimidin-7-yl)-2,2-dimethyltetrahydro-4H-cyclopenta[d][1,3]dioxol-4-yl)methyl) amino)propyl)carbamate (175)

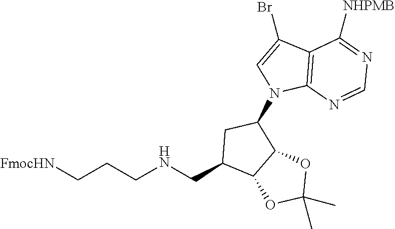

To a solution of compound 166 (0.77 g, 1.52 mmol) in dry DCE (7.50 mL) was added a solution of 52 (0.50 g, 1.70 mmol) in dry DCE (7.50 mL) under nitrogen. The solution was stirred for 30 min at ambient temperature. Then, STAB (0.49 g, 2.28 mmol) was added portion wise over 30 min under nitrogen. The mixture was stirred for 48 h at rt. Then, 1 m K₂CO₃ solution was added to reach pH 8-9. Then, DCM was added, and the organic layer was separated. The water phase was extracted 2 times with DCM. The combined organic layers were washed with brine, dried over sodium sulfate, and concentrated under rotatory evaporation. The crude product was purified over silica (DCM/MeOH; 0-10%) to afford the product as colorless foam (0.50 g, 42%).

### tert-butyl (3-((((9//-fluoren-9-yl) methoxy)carbonyl) amino)propyl)(((3αR,4R,6R,6αS)-6-(5-bromo-4-((4-methoxybenzyl)amino)-7H-pyrrolo[2,3-d]pyrimidin-7-yl)-2,2-dimethyltetrahydro-4H-cyclopenta[d][1,3]dioxol-4-yl)methyl)carbamate (176)

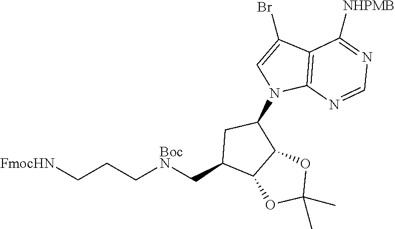

To cooled a solution of compound 175 (0.49 g, 0.63 mmol) in dry CH₂Cl₂ (6.30 mL), di-tert-butyl decarbonate (0.16 mL, 0.69 mmol) was added. The reaction solution was stirred overnight at ambient temperature. After 17 h, reaction mixture was diluted with sodium bicarbonate solution. The organic layer was separated, and the aqueous phase was extracted with CH₂Cl₂. The combined organic layers were washed with brine, dried over sodium sulfate, and concentrated. The obtained residue was purified by flash chromatography (CH₂Cl₂/MeOH; 0-10%) to afford the title product as colorless foam (0.49 g, 89%).

### tert-butyl (3-aminopropyl)(((3αR,4R,6R,6αS)-6-(5-bromo-4-((4-methoxybenzyl)amino)-7H-pyrrolo[2,3-d]pyrimidin-7-yl)-2,2-dimethyltetrahydro-4H-cyclopenta[d][1,3]dioxol-4-yl)methyl)carbamate (177)

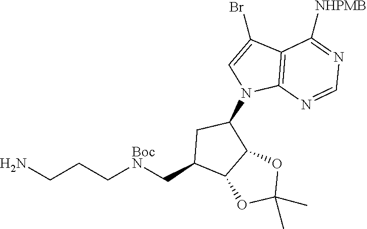

Nucleoside 176 (0.48 g, 0.55 mmol) was dissolved in a 10% piperidine solution in CH₂Cl₂ (11.00 mL). The reaction mixture was stirred for 24 h at ambient temperature. After 24 h, the mixture was diluted with water. The organic phase was separated, and the aqueous phase was extracted with CH₂Cl₂. The combined organic layers were dried over sodium sulfate and concentrated to complete dryness. The crude product was purified over silica (CH₂Cl₂/MeOH; 0-20%) to afford the pure product 177 as colorless solid (0.29 g, 79%).

### tert-butyl (3-(1-(((3αR,4R,6R,6αS)-2,2-dimethyl-6-(4-(methylamino)-7H-pyrrolo[2,3-d]pyrimidin-7-yl) tetrahydro-3aH-cyclopenta[d][1,3]dioxol-4-yl)methyl)-1H-imidazol-2-yl)propyl)(3-phenoxyphenethyl)carbamate

### Synthesis of 4,4-diethoxy-N-(3-phenoxyphenethyl) butan-1-amine (178)

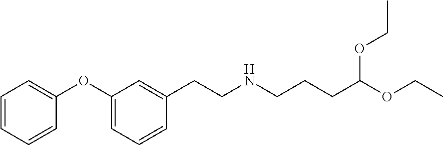 [SMILES: CCOC(CCCNCCc1cccc(Oc2ccccc2)c1)OCC]

A solution of 2-(3-phenoxyphenyl)acetaldehyde (400 mg, 1.9 mmol), 4,4-diethoxybutan-1-amine (911 mg, 5.7 mmol) in MeOH (3 mL) was stirred at room temperature for 30 min. Then NaBH₄ (72 mg, 1.9 mmol) was added to the solution. The solution was stirred at room temperature for 16 hrs. The reaction was quenched with water (2 mL) and diluted with EA (20 mL). The organic phase was washed with H₂O (10 mL×3), dried over Na₂SO₄ and concentrated to dryness. The residue was purified by silica gel flash chromatography (DCM:MeOH=20:1) to give compound 178 (400 mg, yield: 80%) as colorless oil. ¹H NMR (400 MHZ, CD3Cl): 8=7.38 (t,) =7.6 Hz, 2H), 7.28 (t, J=7.6 Hz, 1H), 7.13 (t, J=7.2 Hz, 1H), 7.00-6.98 (m, 3H), 6.87 (s, 1H), 6.80 (dd, J=8.0, 2.0 Hz, 1H), 4.42 (t, J=5.2 Hz, 1H), 3.56-3.49 (m, 2H), 3.43-3.36 (m, 2H), 2.72-2.66 (m, 4H), 2.51-2.48 (m, 2H), 1.52-1.36 (m, 4H), 1.09 (t, J=7.2 Hz, 6H).

### benzyl(4,4-diethoxybutyl)(3-phenoxyphenethyl)carbamate (179)

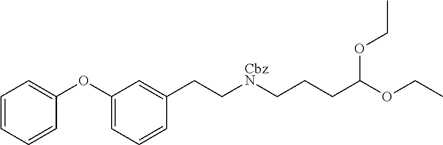 [SMILES: CCOC(CCCN(CCc1cccc(Oc2ccccc2)c1)C(=O)OCc1ccccc1)OCC]

To a solution of 4,4-diethoxy-N-(3-phenoxyphenethyl) butan-1-amine 178 (200 mg, 0.7 mmol) in THF/H₂O (5/1 mL) was added benzyl chloroformate (256 mg, 1.5 mmol) and K₂CO₃ (207 mg, 1.5 mmol), then the solution was stirred at room temperature overnight. The solution was partitioned between water (10 mL) and EA (20 mL). The organic phase was washed with H₂O (10 mL×3), dried over Na₂SO₄ and concentrated to dryness. The residue was purified by silica gel flash chromatography (PE:EA=10:1) to give compound 177 (230 mg, yield: 63%) as colorless oil.

### benzyl(3-oxopropyl)(3-phenoxyphenethyl)carbamate (180)

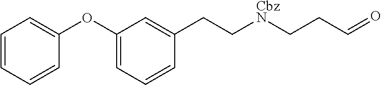 [SMILES: O=CCCN(CCc1cccc(Oc2ccccc2)c1)C(=O)OCc1ccccc1]

To a solution of 179 (230 mg, 0.47 mmol) in THF (5 mL) was added aq HCl (2 mL, 2 M) and the solution was stirred at room temperature for 2 hrs. The reaction solution was partitioned between aq. NH₄Cl (20 mL) EA (20 mL). The organic phase was washed with H₂O (10 mL×3), dried over Na₂SO₄ and concentrated to dryness. The residue was purified by silica gel flash chromatography (PE:EA=4:1) to give compound 180 (160 mg, yield: 82%) as colorless oil. 1H NMR (400 MHZ, CD₃Cl): δ=9.68 (d, J=45.2 Hz, 1H), 7.37-6.79 (m, 14H), 5.12-5.10 (m, 2H), 3.44 (s, 2H), 3.23-3.18 (m, 2H), 2.85-2.78 (m, 2H), 2.44-2.35 (m, 2H), 1.83-1.77 (m, 2H), 2.62 (t, J=8.4 Hz, 2H), 1.33-1.28 (m, 3H).

### benzyl(3-(1-(((3αR,4R,6R,6αS)-2,2-dimethyl-6-(4-(methylamino)-7H-pyrrolo[2,3-d]pyrimidin-7-yl) tetrahydro-3aH-cyclopenta[d][1,3]dioxol-4-yl)methyl)-1H-imidazol-2-yl)propyl)(3-phenoxyphenethyl)carbamate (181)

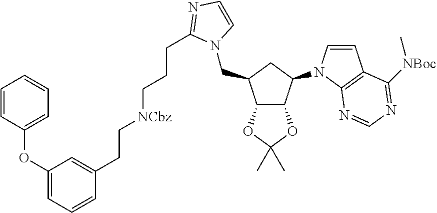 [SMILES: CN(C(=O)OC(C)(C)C)c1ncnc2c1ccn2[C@@H]1C[C@H](Cn2ccnc2CCCN(CCc2cccc(Oc3ccccc3)c2)C(=O)OCc2ccccc2)[C@H]2OC(C)(C)O[C@H]21]

To a solution of 180 (100 mg, 0.2 mmol), 131 (76 mg, 0.2 mmol) and ammonium acetate (18 mg, 0.2 mmol) in MeOH (5 mL) was added glyoxal (30 mg, 0.2 mmol) and the solution was stirred at 35° C. overnight. The crude was purified by reverse phase flash (MeCN/H₂O) to afford 181 (30 mg, yield: 17%) as a white solid. MS Calc.: 755.3; MS Found: 756.1 [M+H⁺].

### tert-butyl (3-(1-(((3αR,4R,6R,6αS)-2,2-dimethyl-6-(4-(methylamino)-7H-pyrrolo[2,3-d]pyrimidin-7-yl) tetrahydro-3aH-cyclopenta[d][1,3]dioxol-4-yl)methyl)-1H-imidazol-2-yl)propyl)(3-phenoxyphenethyl)carbamate (182)

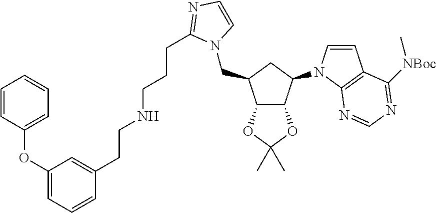 [SMILES: CN(C(=O)OC(C)(C)C)c1ncnc2c1ccn2[C@@H]1C[C@H](Cn2ccnc2CCCNCCc2cccc(Oc3ccccc3)c2)[C@H]2OC(C)(C)O[C@H]21]

To a solution of 181 (10 mg, 0.01 mmol) in EA (3 mL) was added Pd/C(10 mg) and Boc₂O (5 mg, 0.02 mmol). The suspension was stirred at room temperature under H2 for 16 hrs. The suspension is filtered and concentrated for the use of next step. MS Calcd.: 721.4; MS Found: 722.4 [M+H⁺].

### Preparation of tert-butyl (((3αR,4R,6R,6αR)-6-(6-((tert-butoxycarbonyl)amino)-9H-purin-9-yl)-2,2-dimethyltetrahydrofuro[3,4-d][1,3]dioxol-4-yl)methyl)(3-((4-(prop-2-yn-1-yloxy)benzyl) amino)propyl)carbamate

### tert-butyl (((3αR,4R,6R,6αR)-6-(6-((tert-butoxycarbonyl)amino)-9H purin-9-yl)-2,2-dimethyltetrahydrofuro[3,4-d][1,3]dioxol-4-yl)methyl)(3-((4-(prop-2-yn-1-yloxy)benzyl) amino)propyl)carbamate (183)

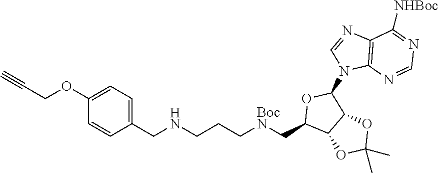 [SMILES: C#CCOc1ccc(CNCCCN(C[C@H]2O[C@@H](n3cnc4c(NC(=O)OC(C)(C)C)ncnc43)[C@@H]3OC(C)(C)O[C@@H]32)C(=O)OC(C)(C)C)cc1]

4-(prop-2-yn-1-yloxy)benzaldehyde (0.06 g, 0.35 mmol) was dissolved in dry DCE under nitrogen. Then, a solution of compound tert-butyl (3-aminopropyl)(((3αR,4R,6R,6αR)-6-(6-((tert-butoxycarbonyl)amino)-9H-purin-9-yl)-2,2-dimethyltetrahydrofuro[3,4-d][1,3]dioxol-4-yl)methyl)carbamate (0.18 g, 0.31 mmol) was added. The solution was stirred for 30 min at rt. STAB (0.10 g, 0.47 mmol) was then added portion wise over 30 min. The mixture was stirred for 48 h at rt. 1M K₂CO₃ solution was added to reach pH 8-9. DCM was added and separated. The aqueous phase was extracted with DCM. The combined organic layers were dried over sodium sulfate, and concentrated. The crude product was purified by FC(CH₂Cl₂/MeOH; 0-10%) to afford the product as white foam (0.14 g, 64%).

### Preparation of tert-butyl (3-((((3αR,4R,6R,6αR)-6-(6-((tert-butoxycarbonyl)amino)-9H-purin-9-yl)-2,2-dimethyltetrahydrofuro[3,4-d][1,3]dioxol-4-yl)methyl) amino)propyl)(4-ethynylphenethyl)carbamate

### tert-butyl (3-((((3αR,4R,6R,6αR)-6-(6-((tert-butoxycarbonyl)amino)-9H purin-9-yl)-2,2-dimethyltetrahydrofuro[3,4-d][1,3]dioxol-4-yl)methyl) amino)propyl)(4-ethynylphenethyl)carbamate (184)

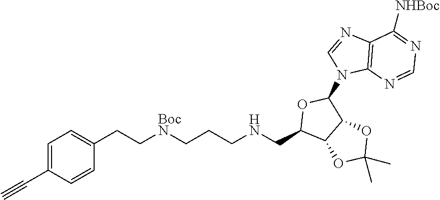 [SMILES: C#Cc1ccc(CCN(CCCNC[C@H]2O[C@@H](n3cnc4c(NC(=O)OC(C)(C)C)ncnc43)[C@@H]3OC(C)(C)O[C@@H]32)C(=O)OC(C)(C)C)cc1]

Compound tert-butyl (4-ethynylphenethyl)(3-oxopropyl)carbamate (0.08 g, 0.26 mmol) was dissolved in dry DCE under nitrogen. Then, a solution of tert-butyl (9-((3αR,4R,6R,6αR)-6-(aminomethyl)-2,2-dimethyltetrahydrofuro[3,4-d][1,3]dioxol-4-yl)-9H-purin-6-yl)carbamate (0.11 g, 0.26 mmol) was added under nitrogen. The solution was stirred for 30 min at rt. STAB (0.08 g, 0.38 mmol) was then added portion wise over 30 min. The mixture was stirred for 48 h at rt. 1M K₂CO₃ solution was added to reach pH 8-9. DCM was added and separated. The aqueous phase was extracted with DCM. The combined organic layers were dried over sodium sulfate, and concentrated. The crude product was purified by FC(CH₂Cl₂/MeOH; 0-10%) to afford the product as white foam (0.08 g, 42%).

### Preparation of 7-((3αS,4R,6R,6αR)-6-(aminomethyl)-2,2-dimethyltetrahydro-4H-cyclopenta[d][1,3]dioxol-4-yl)-5-ethyl-N-(4-methoxybenzyl)-7H-pyrrolo[2,3-d]pyrimidin-4-amine 7-((3αS,4R,6R,6αR)-6-(((tert-butyldimethylsilyl)oxy) methyl)-2,2-dimethyltetrahydro-4H cyclopenta[d][1,3]dioxol-4-yl)-N-(4-methoxybenzyl)-5-vinyl-7//-pyrrolo[2,3-d]pyrimidin-4-amine (185)

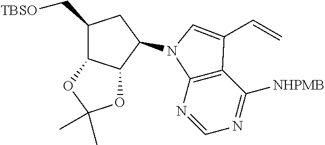

1300 mg (2.10 mmol; 1.00 eq) of 5-bromo-7-((3as,4r,6r,6ar)-6-(((tert-butyldimethylsilyl)oxy) methyl)-2,2-dimethyltetrahydro-4H-cyclopenta[d][1,3]dioxol-4-yl)-N-(4-methoxybenzyl)-7H-pyrrolo[2,3-d]pyrimidin-4-amine were dissolved in 15 ml of Dioxane and 15 ml of dist. H₂O. 0.40 mL (2.31 mmol; 1.10 eq) of Vinylboronicpinacolester, 890 mg (4.20 mmol; 2.00 eq) of K₃PO4 and 137 mg (0.210 mol; 0.10 eq) of Pd (dtbpf) were added under N₂-atmosphere. The resulting reaction mixture was heated to 80° C. for 14 h. Extraction with EtOAc (5×50 mL) followed. The combined organic phases were dried over MgSO₄, filtered and the solvent was removed under reduced pressure. The residue was purified by column chromatography on silica gel (Cyclohexane/EtOAc 100:0% to 50:50%). 400 mg (0.71 mmol; 34%) of the title compound were received in form of a white solid. TLC: R^{f}=0.33 (Cyclohexane/EtOAc 1:1). 1H-NMR: (400 MHZ, DMSO-d₆)}=8.10 (s, 1H), 7.61 (s, 1H), 7.28-7.24 (m, 2H), 7.20 (d, J=10.9 Hz, 1H), 7.16 (d,)=11.0 Hz, 1H), 7.11 (t, J=6.1 Hz, 1H), 6.87-6.83 (m, 2H), 5.55 (dd, J=17.2, 1.7 Hz, 1H), 5.13 (dd, J=10.8, 1.7 Hz, 1H), 4.99-4.95 (m, 1H), 4.94-4.89 (m, 1H), 4.66 (d, J=6.1 Hz, 2H), 4.52-4.48 (m, 1H), 3.70 (s, 3H), 3.68-3.63 (m, 2H), 2.27-2.16 (m, 2H), 2.12-2.03 (m, 1H), 1.46 (s, 3H), 1.21 (s, 3H), 0.88 (s, 9H), 0.05 (s, 6H) ppm. APCI-MS (+) m/z for C₃₁H₄₄N₄O₄Si: calc.: 564.80; found: 565.7 and 566.7.

### 7-((3αS,4R,6R,6αR)-6-(((tert-butyldimethylsilyl)oxy) methyl)-2,2-dimethyltetrahydro-4H-cyclopenta[d][1,3]dioxol-4-yl)-5-ethyl-N-(4-methoxybenzyl)-7H-pyrrolo[2,3-a]pyrimidin-4-amine (186)

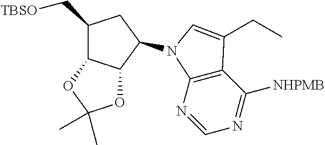

400 mg (0.71 mmol; 1.00 eq) of 7-((3as,4r,6r,6ar)-6-(((tert-butyldimethylsilyl)oxy) methyl)-2,2-dimethyltetrahydro-4H-cyclopenta[d][1,3]dioxol-4-yl)-N-(4-methoxybenzyl)-5-vinyl-7H-pyrrolo[2,3-d]pyrimidin-4-amine were dissolved in 40 ml of Methanol. Catalytic amounts of Pd/C were added and the reaction mixture was flushed by H2-gas with continuous stirring. The catalyst was filtered off over celite and the filtrate was purified by column chormatography on silica gel (Cyclohexane/EtOAc 100:0% to 60:40%). 400 mg (0.71 mmol; quantitative) of the title compound were isolated in form of a colorless foam. TLC: R^{f}=0.48 (Cyclohexane/EtOAc 1:1). 1H-NMR: (400 MHZ, DMSO-d₆) δ=8.05 (s, 1H), 7.30-7.22 (m, 2H), 7.03 (s, 1H), 6.89 (t, J=6.3 Hz, 1H), 6.87-6.82 (m, 2H), 4.96-4.89 (m, 1H), 4.87-4.83 (m, 1H), 4.65 (d, J=6.1 Hz, 2H), 4.51-4.47 (m, 1H), 3.70 (s, 3H), 3.68-3.63 (m, 2H), 2.86-2.80 (m, 2H), 2.26-2.12 (m, 2H), 2.12-2.01 (m, 1H), 1.45 (s, 3H), 1.23 (t, J=7.4 Hz, 3H), 1.20 (s, 3H), 0.88 (s, 9H), 0.05 (s, 6H) ppm. APCI-MS (+) m/z for C₃₁H46N₄O4Si: calc.: 566.82; found: 567.6 and 568.7.

### ((3αR,4R,6R,6αS)-6-(5-ethyl-4-((4-methoxybenzyl)amino)-7//pyrrolo[2,3-d]pyrimidin-7-yl)-2,2-dimethyltetrahydro-4H-cyclopenta[d][1,3]dioxol-4-yl) methanol (187)

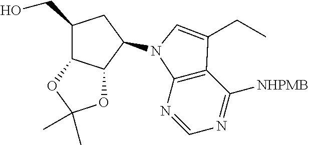

660 mg (1.16 mmol; 1.00 eq) of 7-((3as,4r,6r,6ar)-6-(((tert-butyldimethylsilyl)oxy) methyl)-2,2-dimethyltetrahydro-4H-cyclopenta[d][1,3]dioxol-4-yl)-5-ethyl-N-(4-methoxybenzyl)-7H-pyrrolo[2,3-d]pyrimidin-4-amine were dissolved in 10 ml of THF. 1.80 mL of a 1 m solution of TBAF in THF (1.74 mmol; 1.50 eq) were added and the reaction mixture was stirred at RT overnight. The solvent was removed under reduced pressure and the remaining residue was purified by flash chromatography eluting with Cyclohexane/EtOAc (100%/0% to 50%/50%). 300 mg (0.66 mmol; 57% of the title compound were isolated in form of a white solid. 1H-NMR: (400 MHZ, DMSO-d₆) δ=8.06 (s, 1H), 7.30-7.25 (m, 2H), 7.08 (s, 1H), 6.89 (t, J=6.0 Hz, 1H), 6.86-6.83 (m, 2H), 4.95-4.88 (m, 1H), 4.85-4.81 (m, 1H), 4.72 (t, J=5.3 Hz, 1H), 4.65 (d, J=5.9 Hz, 2H), 4.51-4.48 (m, 1H), 3.70 (s, 3H), 3.54-3.44 (m, 2H), 2.83 (q, J=7.3 Hz, 2H), 2.22-2.09 (m, 2H), 2.03-1.94 (m, 1H), 1.45 (s, 3H), 1.23 (t, J=7.4 Hz, 3H), 1.21 (s, 3H) ppm. APCI-MS (+) m/z for C₂₅H₃₂N₄O₄: calc.: 452.56; found 453.4 and 454.4.

### 2-(((3ar,4r,6r,6as)-6-(5-ethyl-4-((4-methoxybenzyl)amino)-7H-pyrrolo[2,3-d]pyrimidin-7-yl)-2,2-dimethyltetrahydro-4H-cyclopenta[d][1,3]dioxol-4-yl)methyl) isoindoline-1,3-dione (188) and 7-((3ar,4r,6r,6as)-6-(aminomethyl)-2,2-dimethyltetrahydro-4H-cyclopenta[d][1,3]dioxol-4-yl)-5-ethyl-N-(4-methoxybenzyl)-7H-pyrrolo[2,3-d]pyrimidin-4-amine (189)

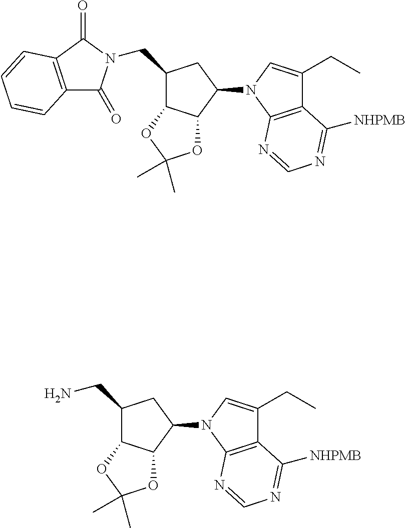

300 mg (0.66 mmol; 1.0 eq) of ((3αR,4R,6R,6as)-6-(5-ethyl-4-((4-methoxybenzyl)amino)-7H-pyrrolo[2,3-d]pyrimidin-7-yl)-2,2-dimethyltetrahydro-4H-cyclopenta[d][1,3]dioxol-4-yl) methanol were dissolved in 20 ml of THF. 226 mg (0.86 mmol; 1.3 eq) of PPh₃, 127 mg (0.86 mmol; 1.3 eq) of phthalimide and finally 0.38 mL (0.86 mmol; 1.3 eq) of a 40% solution of azodicarboxylic acid diethyl ester in toluene were added The reaction solution was stirred at RT overnight and afterwards adsorbed on silica gel. The residue was purified by column chromatography (Cyclohexane/EtOAc 95:5% to 0:100%) and directly used for further reaction. Therefore, the intermediate (320 mg; 0.55 mmol; 1.0 eq) was dissolved in 40 ml of EtOH and 0.43 mL (8.80 mmol; 16 eq) hydrazine hydrate were added. The resulting solution was heated under refluxation for 2 h. The reaction mixture was then cooled to 0° C. and the formed precipitate was filtered off. The residue was washed with EtOH. Final column chromatography on silica gel (DCM/MeOH 100:0% to 80:20%) gave 200 mg (0.44 mmol; 66% over 2 steps) of the title compound in the form of a colorless foam. TLC: R^{f}=0.08 (DCM/MeOH 10:1). 1H-NMR: (400 MHZ, DMSO-d₆) δ=8.06 (s, 1H), 7.30-7.23 (m, 2H), 7.11 (s, 1H), 6.89 (t, J=6.0 Hz, 1H), 6.87-6.81 (m, 2H), 4.94-4.88 (m, 1H), 4.84-4.81 (m, 1H), 4.65 (d, J=5.7 Hz, 2H), 4.49-4.45 (m, 1H), 3.70 (s, 3H), 2.83 (q, J=7.2 Hz, 2H), 2.78-2.61 (m, 2H), 2.24-2.04 (m, 2H), 1.98-1.90 (m, 1H), 1.45 (s, 3H), 1.23 (t, J=7.3 Hz, 3H), 1.21 (s, 3H) ppm. APCI-MS (+) m/z for C₃₃H35N₅O₅: calc.: 581.67; found 582.4 and 583.4. APCI-MS (+) m/z for C₂₅H33N₅O₃: calc.: 451.57; found 452.5 and 453.5.

### Constrained left hand side nucleoside series tert-butyl 4-((3αR,4R,6αR)-2,2-dimethyl-6-oxotetrahydro-4H-cyclopenta[d][1,3]dioxol-4-yl)-3,6-dihydropyridine-1 (2H)-carboxylate (190)

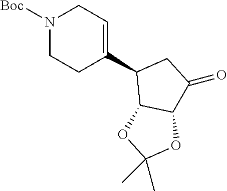 [SMILES: CC(C)(C)OC(=O)N1CC=C([C@H]2CC(=O)[C@@H]3OC(C)(C)O[C@H]23)CC1]

To a heat-dried three-necked round bottom flask equipped with a stirring bar and air condenser was charged with (3αR,6αR)-2,2-dimethyl-3a,6a-dihydro-4H-cyclopenta[d][1,3]dioxol-4-one (0.80 g, 4.93 mmol), [Cp*RhCl₂]₂ (0.25 g, 0.49 mmol), KOH (0.06 g, 0.99 mmol), and tert-butyl 4-(4,4,5,5-tetramethyl-1,3,2-dioxaborolan-2-yl)-3,6-dihydropyridine-1 (2H)-carboxylate (2.36 g, 7.39 mmol) under nitrogen atmosphere, Then, 1,4-dioxane (32.90 mL) was added and the resulted mixture was degassed. Degassed water (1.00 mL) was then added, and the mixture was placed into a pre-heated heating plate (50° C.). The reaction mixture was heated to 80° C. and stirred for 4 h upon complete consumption of the starting material. TLC(cyclohexane/EtOAc; 30%) was used for monitoring the reaction progress. Then, the reaction was cooled down to ambient temperature and was diluted with water. The aqueous phase was extracted three times with EtOAc and the combined organic layers were washed with brine, dried over sodium sulfate, and concentrated under reduced pressure. The obtained crude product was purified by flash chromatography (cyclohexane/EtOAc; 0-20%) to obtain the desired product as colorless oil (1.40 g, 84%). R^{f}=0.37 (petrol ether/EtOAc; 30%). ¹H NMR (400 MHZ, Chloroform-d) 8 5.31-5.20 (m, 1H, —NC H2CH—), 4.57 (d, J=5.5 Hz, 1H, H2’), 4.17 (d, J=5.5 Hz, 1H, H1′), 3.82-3.77 (m, 2H, —NC H2CH—), 3.48-3.39 (m, 2H, —NC H2CH₂—), 2.86 (d, J=9.4 Hz, 1H, H3′), 2.83-2.75 (m, 1H, H4′), 2.28-2.21 (m, 1H, H4′), 2.20-1.94 (m, 2H, —NC H2CH₂—), 1.40 (s, 9H, —NCO₂C(CH₃)₃), 1.39 (s, 3H, CH₃, acetonide), 1.28 (s, 3H, CH₃, acetonide). APCI: calc. for C₁₈H₂₈NO₅ [M+H]⁺: 338.19, found: 238.20 [M+H, Boc cleaved]⁺.

### tert-butyl 4-((3αR,4R,6S,6αS)-6-hydroxy-2,2-dimethyltetrahydro-4H-cyclopenta[d][1,3]dioxol-4-yl)-3.6-dihydropyridine-1 (2)-carboxylate (191)

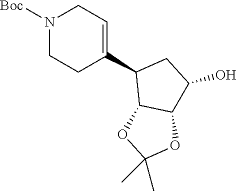 [SMILES: CC(C)(C)OC(=O)N1CC=C([C@H]2C[C@H](O)[C@@H]3OC(C)(C)O[C@@H]32)CC1]

Compound 190 (2.71 g, 6.43 mmol) was dissolved in anhydrous MeOH (32.20 mL) and cooled down to 0° C. Then, NaBH₄ (0.37 g, 9.65 mmol) was added portion wise. The reaction mixture was stirred for 1 h at 0° C. (until the bubbling stopped). Upon full conversion indicated by TLC (cyclohexane/EtOAc; 40%), cold water was added. The aqueous phase was extracted five times with CH₂Cl₂. The combined organic layers were dried over sodium sulfate and concentrated under reduced pressure. The obtained residue was purified with silica gel chromatography (cyclohexane/EtOAc; 0-40%) to afford the pure product as colorless resin (1.68 g, 77%). ¹H NMR (400 MHZ, DMSO-d₆) δ 5.34 (s, 1H, —NC H₂CH—), 4.44-4.40 (m, 1H, H3′), 4.38-4.33 (m, 2H, H2′, —CHOH), 3.90 (ddd, J=11.0, 9.1, 5.5 Hz, 1H, H1′), 3.78 (s, 2H, —NC H₂CH—), 3.46-3.34 (m, 2H, —NC H₂CH₂—), 2.45-2.39 (m, 1H, H4′), 2.07-1.98 (m, 2H, —NC H₂CH₂—), 1.82 (dt, J=12.5, 8.2 Hz, 1H, H5′), 1.69 (dt, J=12.5, 5.0 Hz, 1H. H5′), 1.41 (s, 12H, —NCO₂C(CH₃)₃, CH₃, acetonide), 1.25 (s, 3H, CH₃, acetonide). APCI: calc. for C₁₈H30NO₅ [M+H]⁺: 340.20, found: 239.90 [M+H, Boc cleaved]⁺. tert-butyl 4-((3αR,4R,6S,6αS)-6-hydroxy-2,2-dimethyltetrahydro-4H/-cyclopenta[d][1,3]dioxol-4-yl) piperidine-1-carboxylate (192)

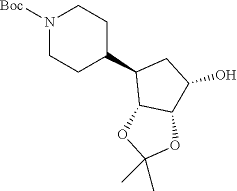 [SMILES: CC(C)(C)OC(=O)N1CCC([C@H]2C[C@H](O)[C@@H]3OC(C)(C)O[C@@H]32)CC1]

Compound 191 (1.65 g, 4.81 mmol) was dissolved in MeOH (24.10 mL) under nitrogen atmosphere. Then, palladium on activated charcoal moistened with water (0.51 g, 10 mol %) was added and the mixture was degassed. The reaction mixture was then purged with hydrogen (balloon) and stirred upon complete consumption. After 17 h, the reaction mixture was set under nitrogen atmosphere and was filtered over celite. The filter cake was rinsed with MeOH and the filtrate was concentrated under reduced pressure. The obtained crude product was purified over silica gel chromatography (cyclohexane/EtOAc; 0-40%) to afford the title product as colorless resin (2.20 g, 72%). R^{f}=0.45 (petrol ether/EtOAc; 50%). ¹H NMR (400 MHZ, Chloroform-d) 8 4.40 (dd, J=7.1, 5.0 Hz, 1H, H2′), 4.29 (dd, J=7.1, 3.6 Hz, 1H, H3′), 4.09-3.99 (m, 2H, —N(CH₂)₂(CH₂)₂—), 3.99-3.94 (m, 1H, H1′), 2.67-2.52 (m, 2H, —N(CH₂) 2 (CH₂) 2—), 2.47 (s, 1H, —CHOH), 2.01-1.86 (m, 2H, H4, H5′), 1.77-1.68 (m, 1H, —N(CH₂) 2 (CH₂) 2—), 1.58-1.46 (m, 3H, N (CH₂) 2 (CH₂) 2, H5′), 1.45 (s, 3H, CH₃, acetonide), 1.38 (s, 9H, —NCO₂C(CH₃)₃), 1.29 (s, 3H, CH₃, acetonide), 1.20-1.01 (m, 2H, —N(CH₂) 2 (CH₂) 2CH—). APCI: calc. for C₁₈H32NO₅ [M+H]⁺: 342.22, found: 242.00 [M+H, Boc cleaved]⁺.

### tert-butyl 4-((3αR,4R,6R,6αS)-6-(4-chloro-7H-pyrrolo[2,3-d]pyrimidin-7-yl)-2,2-dimethyltetrahydro-4H-cyclopenta[d][1,3]dioxol-4-yl) piperidine-1-carboxylate (193)

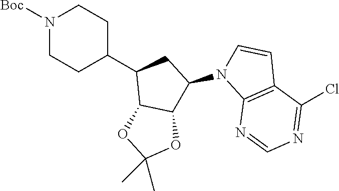 [SMILES: CC(C)(C)OC(=O)N1CCC([C@H]2C[C@@H](n3ccc4c(Cl)ncnc43)[C@@H]3OC(C)(C)O[C@@H]32)CC1]

Following the general procedure B for Mitsunobu-type glycosylation, compound x was obtained starting from 4-chloro-7H-pyrrolo[2,3-d]pyrimidine (0.32 g, 2.03 mmol), compound 192 (0.50 g, 1.45 mmol), PPh₃ (0.77 g, 2.90 mmol), and DIAD (0.53 mL, 2.61 mmol) in dry THF (0.15 M, 9.70 mL) after column chromatography on silica (petrol ether/THF; 0-20%) as white foam (336 mg, 49%). APCI: calc. for C₂₄H₃₄ClN₄O₄ [M+H]⁺: 476.22, found: 476.9/478.9 [M+H]⁺.

### tert-butyl 4-((3αR,4R,6R,6αS)-6-(4-amino-7//-pyrrolo[2,3-d]pyrimidin-7-yl)-2,2-dimethyltetrahydro-4H-cyclopenta[d][1,3]dioxol-4-yl) piperidine-1-carboxylate (194)

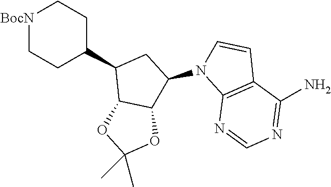 [SMILES: CC(C)(C)OC(=O)N1CCC([C@H]2C[C@@H](n3ccc4c(N)ncnc43)[C@@H]3OC(C)(C)O[C@@H]32)CC1]

Following the general procedure for aromatic substitution, compound x was obtained starting from compound 193 (0.12 g, 0.25 mmol) in a mixture of 1,4-dioxane and ammonia (0.10 M, 2.40 mL, 1:2) after column chromatography on silica (n-heptane/EtOAc; 0-80%) as white foam (177 mg, 94%). APCI: calc. for C₂₄H36N₅O₄ [M+H]⁺: 458.27, found: 458.1 [M+H]⁺. tert-butyl 5-((3αR,4R,6αR)-2,2-dimethyl-6-oxotetrahydro-4H-cyclopenta[d][1,3]dioxol-4-yl)-3,6-dihydropyridine-1 (2H)-carboxylate (195)

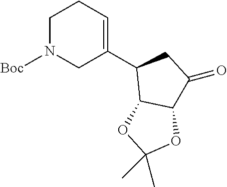 [SMILES: CC(C)(C)OC(=O)N1CCC=C([C@H]2CC(=O)[C@@H]3OC(C)(C)O[C@H]23)C1]

To a heat-dried three-necked round bottom flask equipped with a stirring bar and air condenser was charged with (3αR,6αR)-2,2-dimethyl-3a,6a-dihydro-4H-cyclopenta[d][1,3]dioxol-4-one (1.00 g, 6.16 mmol), [Cp*RhCl₂]₂ (0.31 g, 0.62 mmol), KOH (0.07 g, 1.23 mmol), and tert-butyl 5-(4,4,5,5-tetramethyl-1,3,2-dioxaborolan-2-yl)-3,6-dihydropyridine-1 (2H)-carboxylate (2.95 g, 9.24 mmol) under nitrogen atmosphere, Then, 1,4-dioxane (41.10 mL) was added and the resulted mixture was degassed. Degassed water (1.20 mL) was then added, and the mixture was placed into a pre-heated heating plate (50° C.). The reaction mixture was heated to 80° C. and stirred for 4 h upon complete consumption of the starting material. TLC(cyclohexane/EtOAc; 30%) was used for monitoring the reaction progress. Then, the reaction was cooled down to ambient temperature and was diluted with water. The aqueous phase was extracted three times with EtOAc and the combined organic layers were washed with brine, dried over sodium sulfate, and concentrated under reduced pressure. The obtained crude product was purified by flash chromatography (cyclohexane/EtOAc; 0-20%) to obtain the desired product as colorless oil (1.72 g, 83%). APCI: calc. for C₁₈H28NO₅ [M+H]⁺: 338.19, found: 238.20 [M+H, Boc cleaved]⁺.

### tert-butyl 5-((3αR,4R,65,6αS)-6-hydroxy-2,2-dimethyltetrahydro-4H-cyclopenta[d][1,3]dioxol-4-yl)-3,6-dihydropyridine-1 (2H)-carboxylate (196)

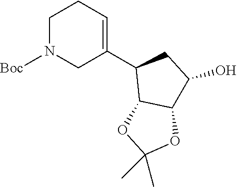 [SMILES: CC(C)(C)OC(=O)N1CCC=C([C@H]2C[C@H](O)[C@@H]3OC(C)(C)O[C@@H]32)C1]

Compound 195 (3.92 g, 11.51 mmol) was dissolved in anhydrous MeOH (57.60 mL) and cooled down to 0° C. Then, NaBH₄ (0.66 g, 17.27 mmol) was added portion wise. The reaction mixture was stirred for 1 h at 0° C. (until the bubbling stopped). Upon full conversion indicated by TLC (cyclohexane/EtOAc; 40%), cold water was added. The aqueous phase was extracted five times with CH₂Cl₂. The combined organic layers were dried over sodium sulfate and concentrated under reduced pressure. The obtained residue was purified with silica gel chromatography (cyclohexane/EtOAc; 0-40%) to afford the pure product as colorless resin (3.54 g, 91%).

### tert-butyl 3-((3αR,4R,65,6αS)-6-hydroxy-2,2-dimethyltetrahydro-4H/-cyclopenta[d][1,3]dioxol-4-yl) piperidine-1-carboxylate (197)

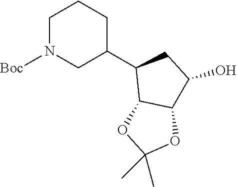 [SMILES: CC(C)(C)OC(=O)N1CCCC([C@H]2C[C@H](O)[C@@H]3OC(C)(C)O[C@@H]32)C1]

Compound 196 (0.02 g, 0.05 mmol) was dissolved in MeOH ( ) under nitrogen atmosphere. Then, palladium on activated charcoal moistened with water (0.01 g, 10 mol %) was added and the mixture was degassed. The reaction mixture was then purged with hydrogen (balloon) and stirred upon complete consumption. After 17 h, the reaction mixture was set under nitrogen atmosphere and was filtered over celite. The filter cake was rinsed with MeOH and the filtrate was concentrated under reduced pressure. The obtained crude product was purified over silica gel chromatography (cyclohexane/EtOAc; 0-50%) to afford the title product as colorless resin ( ) APCI: calc. for C₁₈H32NO₅ [M+H]⁺: 342.22, found:

### 4-iodo-7H-pyrrolo[2,3-d]pyrimidine (198)

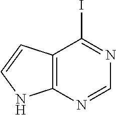

A mixture of 4-chloro-7H-pyrrolo[2,3-d]pyrimidine (3.00 g, 19.34 mmol) and hydroiodic acid (57%, 4.34 g, 19.34 mmol) was stirred at room temperature overnight. The resulted solid was filtered off, and suspended with water. The suspension was cooled down to 0° C. and the pH was subsequently adjusted to 8 by adding aqueous ammonia solution. The solid was filtered off, washed with cold water and dried to complete dryness to afford the title product as beige solid (4191 mg, 88%).

### 7H-pyrrolo[2,3-d]pyrimidine (199)

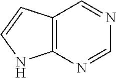

Compound 198 (2.00 g, 8.08 mmol) was dissolved in MeOH (40.40 mL) under nitrogen atmosphere. Then, Et₃N (1.71 mL, 12.12 mmol) was added, and the resulted solution was degassed. Palladium on activated charcoal moistened with water (1.72 g, 0.81 mmol) was added and the mixture was degassed. Afterwards the reaction mixture was purged with hydrogen (ballon) and stirred at ambient temperature overnight. After 17 h, the reaction mixture was purged with nitrogen and filtered over celite. The filtrate was concentrated under reduced pressure. The obtained residue was purified over silica (cyclohexane/EtOAc; 0-100%) to afford the pure product (853 mg, 89%).

### 5-iodo-7H-pyrrolo[2,3-d]pyrimidine (200)

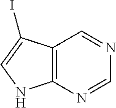

Compound 199 (0.84 g, 7.00 mmol) and NIS (1.67 g, 7.35 mmol) was stirred in dry MeCN (11.70 mL under nitrogen atmosphere. The solution was concentrated to complete dryness and the obtained crude product was purified by flash chromatography (cyclohexane/EtOAc; 0-100%) to afford the title product (1.44 g, 84%).

### 4-iodo-7-((2-(trimethylsilyl) ethoxy)methyl)-7H-pyrrolo[2,3-a]pyrimidine (201)

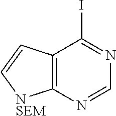 [SMILES: C[Si](C)(C)CCOCn1ccc2c(I)ncnc21]

4-iodo-7H-pyrrolo[2,3-a]pyrimidine (2.16 g, 7.52 mmol) was dissolved in dry DMF (25.10 mL) and cooled down to 0° C. The solution was treated portion wise with sodium hydride (60% in mineral oil, 0.33 g, 8.27 mmol) and stirred for 15 min. Then, a solution of SEMCl (1.41 mL, 7.89 mmol) in DMF was added and the mixture was stirred for 1 h. The mixture was partitioned between EtOAc and water. The organic layer was separated and the aqueous phase was extracted with EtOAc. The combined organic layers were washed with brine, dried over sodium sulfate, and concentrated under reduced pressure. The crude product was purified by flash chromatography (cyclohexane/EtOAc; 0-30%) to afford the title product as white soid (2.36 g, 84%).

### ethyl 2,2-difluoro-2-(7-((2-(trimethylsilyl) ethoxy)methyl)-7H-pyrrolo[2,3-d]pyrimidin-4-yl)acetate (202)

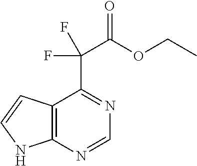 [SMILES: CCOC(=O)C(F)(F)c1ncnc2[nH]ccc12]

Compound 201 (2.00 g, 5.28 mmol) was added to a heat dried three-necked round bottom flask under nitrogen atmosphere. Then, Cul (1.22 g, 6.33 mmol), CsF (1.13 g, 7.39 mmol), and dry DMSO (17.60 mL) was added. The resulted mixture was degassed and at last TMSCF2CO₂Et (2.07 mL, 10.55 mmol) was added. The reaction mixture was stirred at 70° C. for 18 h. Then, the reaction mixture was allowed to cool down to rt and then diluted with water and EtOAc. The organic layer was separated, and the aqueous phase was extracted with EtOAc. The combined organic layers were washed with brine, dried over sodium sulfate, and concentrated. The obtained residue was purified over silica (cyclohexane/EtOAc; 0-20%) to afford the title product (867 mg, 44%).

### 4-(difluoromethyl)-7-((2-(trimethylsilyl) ethoxy)methyl)-7H-pyrrolo[2,3-d]pyrimidine (203)

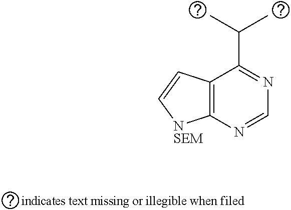 [SMILES: CC(C)c1ncnc2c1ccn2COCC[Si](C)(C)C]

To a solution of compound 202 (0.85 g, 2.27 mmol) in MeOH (2.30 mL) was added a 2 M sodium carbonate solution (4.53 mL, 9.06 mmol) and stirred at ambient temperature for 2 h. The reaction mixture was concentrated, and the obtained residue was taken up in water and the pH was adjusted to 4 with a 1 M HCl solution. The aqueous phase extracted with EtOAc. . . . The combined organic layers were dried over sodium sulfate and concentrated to complete dryness. The obtained residue was redissolved in DMF (26.30 mL). Then, KF (0.80 g, 13.59 mmol) was added and the resulted mixture was heated to 140° C. for 1 h. The mixture was allowed to cool down to rt and extracted with EtOAc. The combined organic layers were washed with brine, dried over sodium sulfate, and concentrated. The crude product was purified over silica gel chromatography (650 mg, 96%).

### 4-(difluoromethyl)-7H-pyrrolo[2,3-d]pyrimidine (204)

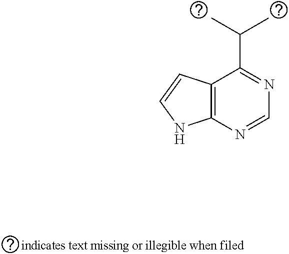

To a solution of compound 203 (0.64 g, 2.11 mmol) in CH₂Cl₂ (5.20 mL) was added TFA (5.20 mL). The reaction mixture was stirred at room temperature for 4 h and then concentrated to complete dryness. The obtained residue was dissolved in MeOH (10.50 mL) and then ammonia solution (2.19 mL, 29.32 mmol) and ethylenediamine (0.07 mL, 1.06 mmol) were added. The reaction mixture was stirred for 2 h and concentrated. The obtained residue was diluted with water and pH was adjusted to 8. The aqueous phase was extracted with CH₂Cl₂. The combined organic layers were dried over sodium sulfate and concentrated to obtain the title product (261 mg, 73%).

### tert-butyl 2-((3αR,4R,6αR)-2,2-dimethyl-6-oxotetrahydro-4H-cyclopenta[d][1,3]dioxol-4-yl) pyrrolidine-1-carboxylate (205)

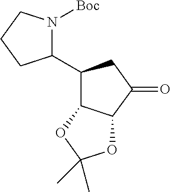 [SMILES: CC(C)(C)OC(=O)N1CCCC1[C@H]1CC(=O)[C@@H]2OC(C)(C)O[C@H]12]

A heat-dried vial was charged with (−)-(3αR,6αR)-3a,6a-Dihydro-2,2-dimethyl-4H-cyclopenta-1,3-dioxol-4-one (0.20 g, 1.23 mmol), Boc-Pro-OH (0.80 g, 3.70 mmol), and anhydrous CsF (0.57 g, 3.70 mmol) under nitrogen atmosphere. Then, dry DMSO (12.30 mL, 0.10 M) was added followed by addition of 3 mol % (Ir[dF (CF3) ppy]2 (dtbpy)) PF6 (0.04 g, 0.04 mmol). The resulted solution was degassed for 15 min. Then, the reaction vial was irradiated by blue LED light at 467 nm at ambient temperature for 17 h. The reaction progress was monitored by TLC(petrol ether/EtOAc; 40%). Then, the reaction mixture was diluted with water and extracted with EtOAc (3×50 mL). The combined organic layers were washed with brine (3×50 mL), dried over sodium sulfate, and concentrated under reduced pressure. The obtained residue was purified by flash chromatography (cyclohexane/EtOAc; 0-30%) to afford the title compound as colorless resin (425 mg, 85%, diastereomeric mixture). R^{f}=0.51 (cyclohexane/EtOAc; 30%). ¹H NMR (400 MHZ, CDCl₃) δ4.95-4.80 (m, 1H, H1′), 4.70 (d, J=5.2 Hz, 1H, H1′), 4.47-4.38 (m, 1H, H2′), 4.34-4.23 (m, 1H, H2′), 4.08-3.97 (m, 1H, —CH₂CH₂CHCH—), 3.74-3.66 (m, 1H, —CH₂CH₂CHCH—), 3.57-3.39 (m, 2H, —NC H₂CH₂CH₂—), 3.38-3.28 (m, 1H, —NC H₂CH₂CH₂—), 3.24-3.09 (m, 1H, —NC H₂CH₂CH₂—), 2.75-2.67 (m, 1H, H4′), 2.67-2.59 (m, 1H, H4′), 2.54-2.43 (m, 2H, H3′), 2.30-2.18 (m, 1H, H4′), 2.15-2.05 (m, 1H, H4′), 1.97-1.78 (m, 5H, —NC H₂CH₂CH₂—), 1.72-1.56 (m, 2H, —NC H₂CH₂CH₂—), 1.46 (s, 9H, —NCO₂C(CH₃)₃), 1.45-1.41 (m, 12H, —NHCO₂C(CH₃)₃, CH₃, acetonide), 1.41 (s, 3H, CH₃, acetonide), 1.35-1.33 (m, 3H, CH₃, acetonide), 1.32 (s, 3H, CH₃, acetonide). HRMS calcd.: 325.19; found: 348.19 [M+H]⁺.

### tert-butyl 2-((3αR,4R,6αR)-2,2-dimethyl-6-oxotetrahydro-4H-cyclopenta[d][1,3]dioxol-4-yl) piperidine-1-carboxylate (206)

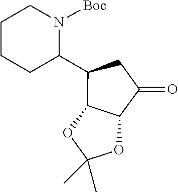 [SMILES: CC(C)(C)OC(=O)N1CCCCC1[C@H]1CC(=O)[C@@H]2OC(C)(C)O[C@H]12]

A heat-dried vial was charged with (−)-(3αR,6αR)-3a,6a-Dihydro-2,2-dimethyl-4H-cyclopenta-1,3-dioxol-4-one (0.25 g, 1.54 mmol), Boc-Pip-OH (1.07 g, 4.62 mmol), and anhydrous CsF (0.71 g, 4.62 mmol) under nitrogen atmosphere. Then, dry DMSO (7.70 mL, 0.20 M) was added followed by addition of 3 mol % (Ir[dF (CF3) ppy]2 (dtbpy)) PF6 (0.05 g, 0.05 mmol). The resulted solution was degassed for 15 min. Then, the reaction vial was irradiated by blue LED light at 467 nm at ambient temperature for 17 h. The reaction progress was monitored by TLC(petrol ether/EtOAc; 30%). Then, the reaction mixture was diluted with water and extracted with EtOAc (3×50 mL). The combined organic layers were washed with brine (3×50 mL), dried over sodium sulfate, and concentrated under reduced pressure. The obtained residue was purified by flash chromatography (cyclohexane/EtOAc; 0-30%) to afford the title compound as colorless resin (435 mg, 94%, d.r. 2.3:1). R^{f}=0.54 (petrol ether/EtOAc; 30%). ¹H NMR (400 MHZ, CDCl₃) δ4.53-4.47 (m, 2H, H1′), 4.43-4.30 (m, 2H, H2′), 4.10-3.91 (m, 2H, —CH₂CH₂CHCH—), 2.98-2.67 (m, 2H, H3′), 2.64-2.46 (m, 2H, H4′, —NC HC H₂—), 2.27-2.07 (m, 2H, H4′), 1.95-1.83 (m, 2H, —NC H₂CH₂—), 1.75-1.54 (m, 12H, —NC H₂CH₂CH₂CH₂—), 1.46 (s, —NHCO₂C(CH₃)₃), 1.45 (s, 3H, CH₃, acetonide), 1.43 (s, 3H, CH₃, acetonide), 1.41 (s, —NHCO₂C(CH₃)₃), 1.35 (s, 3H, CH₃, acetonide), 1.30 (s, 3H, CH₃, acetonide). HRMS calcd.: 339.20; found: 378.19 [M+Na]⁺.

### tert-butyl (3-(4,4,5,5-tetramethyl-1,3,2-dioxaborolan-2-yl)benzyl)carbamate (207)

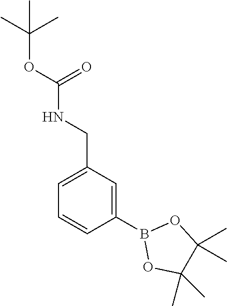 [SMILES: CC(C)(C)OC(=O)NCc1cccc(B2OC(C)(C)C(C)(C)O2)c1]

A three-necked round bottom flask was charged with tert-butyl (3-bromobenzyl)carbamate (5.00 g, 17.30 mmol), Kac (5.15 g, 51.89 mmol), B₂Pin₂ (5.32 g, 20.76 mmol), and Pd (dppf) Cl₂ (1.28 g, 1.73 mmol) under nitrogen atmosphere. Then, 1,4-dioxane (0.30 M, 57.70 mL) was added, and the resulted mixture was degassed. Then, the mixture was heated to 80° C. and stirred for 17 h. before, it was diluted with water and extracted with EtOAc. The combined organic layers were washed with water, dried over sodium sulfate, and concentrated under vacuo. The crude product was purified over silica gel chromatography (cyclohexane/EtOAc; 0-20%) to afford the desired compound x as white solid (5.49 g, 95%). ¹H NMR (400 MHZ, DMSO-d₆) δ7.58 (s, 1H, H6 aryl), 7.55-7.51 (m, 1H, H4 aryl), 7.40 (t, J=6.1 Hz, 1H, —NHCO₂—), 7.37-7.30 (m, 2H, H2, H3 aryl), 4.13 (d, J=6.2 Hz, 2H, —CH₂NH—), 1.40 (s, 9H, —NHCO₂C(CH₃)₃), 1.30 (s, 12H, —BO₂(C(CH₃)₂)₂). APCI: calc. for C₁₈H29BNO₄ [M+H]⁺: 334.21, found: 567.2 [2M+H-Boc]⁺.

### tert-butyldimethyl(3-(4,4,5,5-tetramethyl-1,3,2-dioxaborolan-2-yl)phenoxy) silane (208)

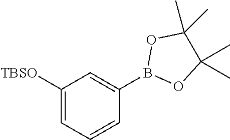 [SMILES: CC1(C)OB(c2cccc(O[Si](C)(C)C(C)(C)C)c2)OC1(C)C]

To a solution of 3-(4,4,5,5-tetramethyl-1,3,2-dioxaborolan-2-yl)phenol (1.21 g, 5.52 mmol, 1.0 eq.) in dry DMF (12 mL) was added imidazole (938.6 mg, 13.79 mmol, 2.6 eq.), then TBSCl (984.9 mg, 6.54 mmol, 1.2 eq.). The solution was stirred for 18.6 h at RT, then the reaction mixture was quenched with ice water and extracted two times with EtOAc. Afterwards the combined organic layers were washed with brine, dried over Na₂SO₄, filtered and concentrated under reduced pressure. The residue was purified by flash chromatography eluting with Cyclohexane/EtOAc (0-+1%) to afford tert-butyldimethyl(3-(4,4,5,5-tetramethyl-1,3,2-dioxaborolan-2-yl)phenoxy) silane (yield: 1.43 g, 4.29 mmol, 79%) as a transparent oil. R^{f}=0.62 (Cyclohexane/EtOAc 96:4). APCI: calcd. for C₁₈H₃₁BO₃Si [M+H]⁺: 335.2 found 335.1. ¹H NMR (400 MHz, DMSO-d₆) δ 7.30-7.25 (m, 2H, o-H, m-H), 7.09-7.07 (m, 1H, p-H), 7.00-6.95 (m, 1H, o′ H), 1.28 (s, 12H, 4 x CH₃), 0.95 (s, 9H, C(CH₃)₃), 0.17 (s, 6H, Si (CH₃) 2)

### tert-butyldimethyl(4-(4,4,5,5-tetramethyl-1,3,2-dioxaborolan-2-yl)phenoxy) silane (209)

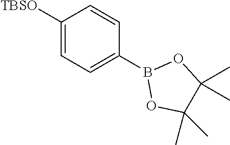 [SMILES: CC1(C)OB(c2ccc(O[Si](C)(C)C(C)(C)C)cc2)OC1(C)C]

To a solution of 3-(4,4,5,5-tetramethyl-1,3,2-dioxaborolan-2-yl)phenol (1.21 g, 5.50 mmol) in dry DMF (12 mL) was added imidazole (934 mg, 13.72 mmol, 2.5 eq.) followed by TBSCl (981 mg, 6.51 mmol, 1.2 eq.). After 18.3 h stirring at rt, more TBSCl (334 mg, 2.21 mmol, 0.4 eq.) was added. The solution was stirred for 24 h, then concentrated to remove DMF. 100 ml ice water was added, then extracted two times with ethyl acetate. The combined organic layers were washed with brine, dried over Na₂SO₄, filtered and concentrated. The residue was purified by flash chromatography eluting with Cyclohexane/EtOAc (0-+10%) to give tert-butyldimethyl(4-(4,4,5,5-tetramethyl-1,3,2-dioxaborolan-2-yl)phenoxy) silane (yield: 1.53 g, 4.58 mmol, 83%), as a transparent oil that solidified after a few days in the fridge. R^{f}=0.31 (Cyclohexane/EtOAc 98:2). APCI: calcd. for C₁₈H31BO₃Si [M+H]⁺: 335.2 found 335.1 ¹H NMR (400 MHZ, DMSO-d₆) δ 7.59-7.56 (m, 2H, 2 x o-H), 6.86-6.83 (m, 2H, 2 x m-H), 1.27 (s, 12H, 4 x CH₃), 0.94 (s, 9H, C(CH₃)₃), 0.18 (s, 6H, Si (CH₃) 2)

### 4,4,5,5-tetramethyl-2-(3-(2-methyl-1,3-dioxolan-2-yl)phenyl)-1,3,2-dioxaborolane (210)

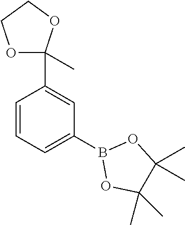 [SMILES: CC1(c2cccc(B3OC(C)(C)C(C)(C)O3)c2)OCCO1]

To a mixture of 1-(3-(4,4,5,5-tetramethyl-1,3,2-dioxaborolan-2-yl)phenyl)ethan-1-one (999 mg, 4.06 mmol, 1 eq.) and ethylene glycol (2.2 mL, 39 mmol, 9.7 equiv) in toluene (40 mL) was added p-tolylsulfonic acid trihydrate (39 mg, 0.2 mmol, 0.05 eq.). The mixture was refluxed (at 140° C.) for 18 h over a Dean-Stark apparatus to remove generated water. Then, the solvent was removed under reduced pressure and the residue was purified by silica chromatography eluting with Cyclohexane/EtOAc (1-+15%) to give 4,4,5,5-tetramethyl-2-(3-(2-methyl-1,3-dioxolan-2-yl)phenyl)-1,3,2-dioxaborolane (yield: 433 mg, 1.49 mmol, 37%) as a white solid. R^{f}=0.25 (Cyclohexane/EtOAc 95:5). APCI: calcd. for C₁₆H23BO₄ [M+H]⁺: 291.2 found 290.8. ¹H NMR (400 MHZ, DMSO-d₆) δ 7.75-7.72 (m, 1H, o-H), 7.61 (dt, J=7.4, 1.3 Hz, 1H, o-H), 7.55-7.52 (m, 1H, p-H), 7.40-7.35 (m, 1H, m-H), 3.98 (td, J=6.1 Hz, 4.2 Hz, 2H, CH₂, ketal), 3.67 (td, J=6.0 Hz, 4.1 Hz, 2H, CH₂, ketal), 1.54 (s, 3H, CH₃. ketal), 1.30 (s, 12H, 4 x CH₃, pinacol ester).

### tert-butyl (4-(4,4,5,5-tetramethyl-1,3,2-dioxaborolan-2-yl)phenyl)carbamate (211)

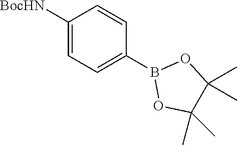 [SMILES: CC(C)(C)OC(=O)Nc1ccc(B2OC(C)(C)C(C)(C)O2)cc1]

2190 mg (10.00 mmol; 1.00 eq) of 4-(4,4,5,5-tetramethyl-1,3,2-dioxaborolan-2-yl) aniline were dissolved in 50 ml of DMF. 2728 mg (12.50 mmol; 1.25 eq) of Boc₂O and 2.77 mL (20.00 mmol; 2.00 eq) of Et₃N were added. The reaction mixture was heated at 90° C. for 72 h. The solvent was removed under reduced pressure and the residue was purified by column chromatography (Petrolether/EtOAc 99:1% to 65:35%) to give 2700 mg (8.46 mmol; 85%) of the title compound in the form of a colorless solid. 1H-NMR: (400 MHZ, DMSO-d₆) δ=9.52 (s, 1H), 7.58-7.51 (m, 2H), 7.50-7.43 (m, 2H), 1.47 (s, 9H), 1.27 (s, 12H) ppm.

### tert-butyl (3-(4.4,5,5-tetramethyl-1,3,2-dioxaborolan-2-yl)phenyl)carbamate (212)

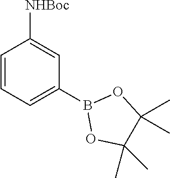 [SMILES: CC(C)(C)OC(=O)Nc1cccc(B2OC(C)(C)C(C)(C)O2)c1]

2190 mg (10.00 mmol; 1.00 eq) of 3-(4,4,5,5-tetramethyl-1,3,2-dioxaborolan-2-yl) aniline were dissolved in 50 ml of DMF. 2728 mg (12.50 mmol; 1.25 eq) of Boc₂O and 2.77 mL (20.00 mmol; 2.00 eq) of Et₃N were added. The reaction mixture was heated at 90° C. overnight. The solvent was removed under reduced pressure and the residue was purified by column chromatography (Petrolether/EtOAc 99:1% to 65:35%) to give 2380 mg (7.45 mmol; 75%) of the title compound in the form of a colorless solid. TLC: R^{f}=0.80 (Petrolether/EtOAc 3:1). 1H-NMR: (400 MHZ, DMSO-d₆) δ=9.32 (s, 1H), 7.90 (s, 1H), 7.48-7.42 (m, 1H), 7.27-7.22 (m, 2H), 1.47 (s, 9H), 1.29 (s, 12H) ppm.

### (3αR,6R,6αR)-6-(3,5-dichlorophenyl)-2,2-dimethyltetrahydro-4H-cyclopenta[d][1,3]dioxol-4-one (213)

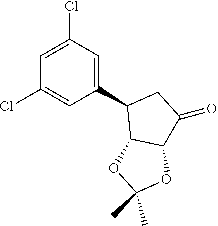

Following the general procedure, compound 213 was obtained starting from (3αR,6αR)-2,2-dimethyl-3a,6a-dihydro-4H-cyclopenta[d][1,3]dioxol-4-one (1.00 g, 6.16 mmol), 2-(3,5-dichlorophenyl)-4,4,5,5-tetramethyl-1,3,2-dioxaborolane (2.55 mg, 9.24 mmol), [Cp*RhCl₂]₂ (0.31 g, 0.62 mmol), and KOH (0.07 g, 1.23 mmol) in dry degassed 1,4-dioxane (0.15 M, 41.10 mL) and degassed water (5.00 M, 1.20 mL) after column chromatography on silica (cyclohexane/EtOAc; 0-20%) as yellowish resin (1190 mg, 64%). R^{f}=0.44 (cyclohexane/EtOAc; 20%). ¹H NMR (400 MHZ, DMSO-d₆) δ7.52 (t, J=1.9 Hz, 1H, p-Ar), 7.40-7.39 (m, 2H, o-Ar), 4.75 (dd, J=6.1, 3.5 Hz, 1H, H1′), 4.66-4.62 (m, 1H, H2′), 3.56 (td, J=8.7, 3.8 Hz, 1H, H3′), 2.90 (dd, J=18.3, 8.6 Hz, 1H, H4′), 2.65 (ddd, J=18.2, 8.0, 1.5 Hz, 1H, H5′), 1.41 (s, 3H, CH₃, acetonide), 1.28 (s, 3H, CH₃, acetonide). APCI: calc. for C₁₄H15Cl₂O3 [M+H]⁺: 301.03, found: 301.20/303.20.

### (6R)-2,2-dimethyl-6-phenyltetrahydro-4H-cyclopenta[d][1,3]dioxol-4-one (214)

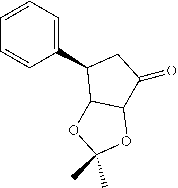

Following the general procedure H, compound 214 was obtained starting from (3αR,6αR)-2,2-dimethyl-3a,6a-dihydro-4H-cyclopenta[d][1,3]dioxol-4-one (1.00 g, 6.16 mmol), 4,4,5,5-tetramethyl-2-phenyl-1,3,2-dioxaborolane (1.95 g, 9.24 mmol), [Cp*RhCl₂]₂ (0.31 g, 0.62 mmol), and KOH (0.07 g, 1.23 mmol) in dry degassed 1,4-dioxane (0.15 M, 41.10 mL) and degassed water (5.00 M, 1.20 mL) after column chromatography on silica (cyclohexane/EtOAc; 0-30%) as yellowish resin (1143 mg, 80%). R^{f}=0.57 (cyclohexane/EtOAc; 30%). ¹H NMR (400 MHZ, DMSO-d₆) δ7.38-7.32 (m, 2H, m-Ar), 7.29-7.25 (m, 1H, p-Ar), 7.25-7.21 (m, 2H, o-Ar), 4.72 (dd, J=5.8, 2.5 Hz, 1H, H1′), 4.57 (dt, J=5.8, 0.9 Hz, 1H, H2′), 3.55 (ddd, J=8.7, 5.8, 2.6 Hz, 1H, H3′), 3.02-2.90 (m, 1H, H4′), 2.54 (dd, J=5.7, 1.7 Hz, 1H, H4′), 1.42-1.39 (m, 3H, CH₃, acetonide), 1.28 (d, J=0.7 Hz, 3H, CH₃, acetonide). APCI: calc. for C₁₄H1603 [M+H]⁺: 233.11, found: 233.20.

### (3αR,6R,6αR)-2,2-dimethyl-6-(naphthalen-2-yl) tetrahydro-4H-cyclopenta[d][1,3]dioxol-4-one (215)

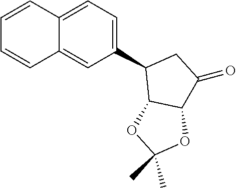

Following the general procedure H, compound 215 was obtained starting from (3αR,6αR)-2,2-dimethyl-3a,6a-dihydro-4H-cyclopenta[d][1,3]dioxol-4-one (1.00 g, 6.16 mmol), 4,4,5,5-tetramethyl-2-(naphthalen-2-yl)-1,3,2-dioxaborolane (1.95 g, 9.24 mmol), [Cp*RhCl₂]₂ (0.31 g, 0.62 mmol), and KOH (0.07 g, 1.23 mmol) in dry degassed 1,4-dioxane (0.15 M, 41.10 mL) and degassed water (5.00 M, 1.20 mL) after column chromatography on silica (cyclohexane/EtOAc; 0-30%) as yellowish resin (1290 mg, 62%). R^{f}=0.47 (cyclohexane/EtOAc; 20%). ¹H NMR (400 MHZ, DMSO-d₆) δ 7.94-7.86 (m, 3H, H4/5/8 naphthyl), 7.73-7.69 (m, 1H, H1, naphthyl), 7.54-7.48 (m, 2H, H3/H6 naphthyl), 7.48-7.43 (m, 1H, H7 naphthyl), 4.85 (dd, J=5.8, 2.5 Hz, 1H, H1′), 4.64 (dt, J=5.8, 0.9 Hz, 1H, H2′), 3.73 (ddd, J=8.7, 5.9, 2.6 Hz, 1H, H3′), 3.08-2.99 (m, 1H, H4′), 2.66 (ddd, J=18.3, 5.8, 1.2 Hz, 1H, H4′), 1.43 (s, 3H, CH₃, acetonide), 1.31 (s, 3H, CH₃, acetonide). APCI: calc. for C₁₈H19O3 [M+H]⁺: 283.13, found: 283.20.

### (3αR,6R,6αR)-2,2-dimethyl-6-(pyridin-4-yl) tetrahydro-4H-cyclopenta[d][1,3]dioxol-4-one (216)

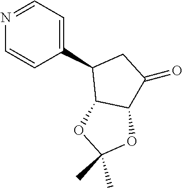

Following the general procedure H, compound 216 was obtained starting from (3αR,6αR)-2,2-dimethyl-3a,6a-dihydro-4H-cyclopenta[d][1,3]dioxol-4-one (1.00 g, 6.16 mmol), 4-(4,4,5,5-tetramethyl-1,3,2-dioxaborolan-2-yl) pyridine (1.95 g, 9.24 mmol), [Cp*RhCl₂]₂ (0.31 g, 0.62 mmol), and KOH (0.07 g 1.23 mmol) in dry degassed 1,4-dioxane (0.15 M, 41.10 mL) and degassed water (5.00 M, 1.20 mL) after column chromatography on silica (cyclohexane/EtOAc; 100%) as yellowish resin (1099 mg, 76%). R^{f}=0.37 (cyclohexane/EtOAc; 100%). ¹H NMR (400 MHZ, DMSO-d₆) δ8.55 -8.51 (m, 2H, H2/6 pyridine), 7.33-7.28 (m, 2H, H3/5, pyridine), 4.75 (dd, J=5.9, 3.1 Hz, 1H, H1′), 4.60 (dt, J=5.9, 0.9 Hz, 1H, H2′), 3.56 (ddd, J=9.4, 6.7, 3.1 Hz, 1H, H3′), 3.01-2.89 (m, 1H), 2.60 (ddd, J=18.3, 6.8, 1.6 Hz, 1H, H4′), 1.41 (s, 3H, CH₃, acetonide), 1.29 (s, 3H, CH₃, acetonide). APCI: calc. for C₁₃H₁₆NO₃ [M+H]⁺: 234.11, found: 234.30.

### (3αR,65,6αR)-2,2-dimethyl-6-(thiophen-2-yl) tetrahydro-4H-cyclopenta[d][1,3]dioxol-4-one (217)

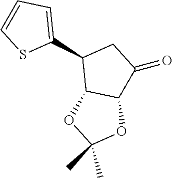

Following the general procedure H, compound 217 was obtained starting from (3αR,6αR)-2,2-dimethyl-3a,6a-dihydro-4H-cyclopenta[d][1,3]dioxol-4-one (1.00 g, 6.16 mmol), 4,4,5,5-tetramethyl-2-(thiophen-2-yl)-1,3,2-dioxaborolane (2.00 g, 9.24 mmol), [Cp*RhCl₂]₂ (0.16 g, 0.31 mmol), and KOH (0.07 g, 1.23 mmol) in dry degassed 1,4-dioxane (0.15 M, 41.10 mL) and degassed water (5.00 M, 1.20 mL) after column chromatography on silica (cyclohexane/EtOAc; 0-30%) as colorless resin (320 mg, 22%). R^{f}=0.27 (cyclohexane/EtOAc; 20%). ¹H NMR (400 MHZ, DMSO-d₆) δ1H NMR (400 MHZ, DMSO-d₆) δ 7.45 (dd, J=5.1, 1.2 Hz, 1H, H5 thiophene), 7.01-6.98 (m, 1H, H4 thiophene), 6.95 (dt, J=3.5, 1.1 Hz, 1H, H3 thiophene), 4.82 (dd, J=5.7, 2.3 Hz, 1H, H1′), 4.46 (ddt, J=5.7, 1.6, 0.8 Hz, 1H, H2′), 3.84-3.78 (m, 1H, H3′), 3.09-3.01 (m, 1H, H4′), 2.58-2.51 (m, 1H, H4′), 1.39 (s, 3H, CH₃, acetonide), 1.30 (s, 3H, CH₃, acetonide). APCI: calc. for C₁₂H₁₅O₃S [M+H]⁺: 239.07, found: 239.10.

### (3αR,6R,6αR)-2,2-dimethyl-6-(1H-pyrazol-5-yl) tetrahydro-4H-cyclopenta[d][1,3]dioxol-4-one (218)

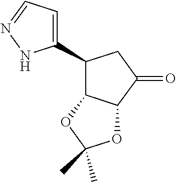

Following the general procedure H, compound 218 was obtained starting from (3αR,6αR)-2,2-dimethyl-3a,6a-dihydro-4H-cyclopenta[d][1,3]dioxol-4-one (0.80 g, 4.93 mmol), tert-butyl 5-(4,4,5,5-tetramethyl-1,3,2-dioxaborolan-2-yl)-1H-pyrazole-1-carboxylate (2.24 g, 7.39 mmol), [Cp*RhCl₂]₂ (0.25 g, 0.49 mmol), and KOH (0.06 g, 0.99 mmol) in dry degassed 1,4-dioxane (0.15 M, 32.90 mL) and degassed water (5.00 M, 1.00 mL) after column chromatography on silica (cyclohexane/EtOAc, 0-60%) as colorless resin (500 mg, 46%). Under standard conditions the boc group was cleaved off. ¹H NMR (400 MHZ, DMSO-d₆) δ7.91 (dd, J=2.3, 0.6 Hz, 1H, H3 pyrazol), 7.49 (d, J=1.8 Hz, 1H, H4 pyrazol), 6.27 (t, J=2.1 Hz, 1H, NH pyrazol), 5.17 (d, J=7.6 Hz, 1H, H1′), 4.72-4.65 (m, 1H, H2′), 4.50-4.45 (m, 1H, H3′), 3.14 (dd, J=18.1, 7.5 Hz, 1H, H4′), 2.39-2.31 (m, 1H, H4′), 1.40 (s, 3H, CH₃, acetonide), 1.28 (s, 3H, CH₃, acetonide). APCI: calc. for C₁₁H₁₅N₂O₃ [M+H]⁺: 223.10, found: 223.20.

### (3αR,6R,6αR)-2,2-dimethyl-6-(quinolin-6-yl) tetrahydro-4H-cyclopenta[d][1,3]dioxol-4-one (219)

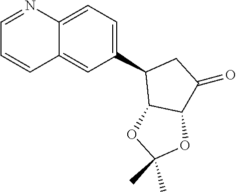

Following the general procedure H, compound 219 was obtained starting from (3αR,6αR)-2,2-dimethyl-3a,6a-dihydro-4H-cyclopenta[d][1,3]dioxol-4-one (0.80 g, 4.93 mmol), 6-(4,4,5,5-tetramethyl-1,3,2-dioxaborolan-2-yl) quinoline (1.95 g, 7.39 mmol), [Cp*RhCl₂]₂ (0.25 g, 0.49 mmol), and KOH (0.06 g, 0.99 mmol) in dry degassed 1,4-dioxane (0.15 M, 32.90 mL) and degassed water (5.00 M, 1.00 mL) after column chromatography on silica (petrol ether/EtOAc; 0-50%) as colorless resin (1066, 76.3%). ¹H NMR (400 MHZ, DMSO-d₆) δ8.89 (dd, J=4.2, 1.7 Hz, 1H, H2 quinolin), 8.34 (ddd, J=8.3, 1.5, 0.6 Hz, 1H, H3 quinolin), 8.03 (d, J=8.7 Hz, 1H, H8 quinolin), 7.81 (d, J=2.0 Hz, 1H, H5 quinolin), 7.73 (dd, J=8.7, 2.1 Hz, 1H, H7 quinolin), 7.54 (dd, J=8.3, 4.2 Hz, 1H, H4 quinolin), 4.87 (dd, J=5.9, 2.7 Hz, 1H, H1′), 4.69-4.65 (m, 1H, H2′), 3.78 (ddd, J=8.9, 6.1, 2.7 Hz, 1H, H3′), 3.10-3.01 (m, 1H, H4′), 2.70 (ddd, J=18.3, 6.1, 1.4 Hz, 1H, H4′), 1.44 (s, 3H, CH₃, acetonide), 1.32 (s, 3H, CH₃, acetonide). APCI: calc. for C₁₇H₁₈NO₃ [M+H]⁺: 284.12, found: 283.90.

### tert-butyl (3-((3αR,4R,6αR)-2,2-dimethyl-6-oxotetrahydro-4H-cyclopenta[d][1,3]dioxol-4-yl)benzyl)carbamate (220)

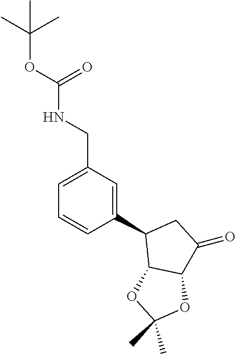

Following the general procedure H, compound 220 was obtained starting from (3αR,6αR)-2,2-dimethyl-3a,6a-dihydro-4H-cyclopenta[d][1,3]dioxol-4-one (1.65 g, 10.17 mmol), compound 207 (5.24 g, 15.25 mmol), [Cp*RhCl₂]₂ (0.51 g, 1.02 mmol), and KOH (0.12 g, 2.03 mmol) in dry degassed 1,4-dioxane (0.15 M, 67.80 mL) and degassed water (5.00 M, 2.00 mL) after column chromatography on silica (n-heptane/EtOAc; 0-50%) as colorless resin (3160 mg, 86%). ¹H NMR (400 MHZ, DMSO-d₆) δ7.31 (t, J=6.0 Hz, 1H—NH), 7.27-7.21 (m, 1H, m-Ar), 7.10-7.01 (m, 3H, o,p-Ar), 4.65 (dd, J=5.8, 2.5 Hz, 1H, H1′), 4.52-4.48 (m, 1H, H2′), 4.06 (d, J=6.1 Hz, 2H, —NHC H2), 3.48 (ddd, J=8.5, 5.6, 2.4 Hz, 1H, H3′), 2.91 (dd, J=18.1, 8.8 Hz, 1H, H4′), 2.48-2.41 (m, 1H, H4′, under DMSO peak) 1.35 (s, 12H, —NCO₂C(CH₃)₃, CH₃, acetonide), 1.23 (s, 3H, CH₃, acetonide). APCI: calc. for C₂₀H₂₈NO₅ [M+H]⁺: 362.19, found: 623.10 [2M+H, —Boc]⁺.

### (3αR,6R,6αR)-6-(3-((tert-butyldimethylsilyl)oxy)phenyl)-2,2-dimethyltetrahydro-4H cyclopenta[d][1,3]dioxol-4-one (221)

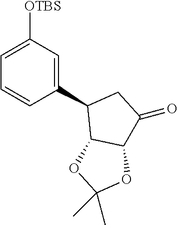 [SMILES: CC1(C)O[C@@H]2[C@@H](c3cccc(O[Si](C)(C)C(C)(C)C)c3)CC(=O)[C@@H]2O1]

Following the general procedure H, compound 221 was obtained starting from (3αR,6αR)-2,2-dimethyl-3a,6a-dihydro-4H-cyclopenta[d][1,3]dioxol-4-one (0.45 g, 2.94 mmol), compound 208 (1.49 g, 4.46 mmol), [Cp*RhCl₂]₂ (0.15 g, 0.30 mmol), and KOH (0.04 g, 0.68 mmol) in dry degassed 1,4-dioxane (10.00 mL) and degassed water (0.50 mL) after column chromatography on silica (cyclohexane/EtOAc; 1-15%) as yellow resin (857 mg, 85%). R^{f}=0.26 (Cyclohexane/EtOAc 9:1). APCI: calcd. for C₂₀H₃₀O₄Si [M+H]⁺: 363.2 found 263.0, 305.0, 363.1. ¹H NMR (400 MHZ, DMSO-d₆) δ 7.23 (t, J=7.9 Hz, 1H, m-H), 6.82-6.80 (m, 1H, o-H), 6.74 (ddd, J=8.1, 2.4, 0.9 Hz, 1H, o-H), 6.70-6.69 (m, 1H, p-H), 4.70 (dd, J=5.8, 2.7 Hz, 1H, H3′), 4.51 (dt, J=5.8 Hz, 1H, H2′), 3.52-3.48 (m, 1H, H4′), 2.94 (dd, J=18.0, 8.9 Hz, 1H, H5′A), 2.47 (m, 1H, H5′B), 1.40 (s, 3H, CH₃, acetonide), 1.28 (s, 3H, CH₃, acetonide), 0.95 (s, 9H, C(CH₃)₃), 0.18 (s, 6H, Si (CH₃) 2)

### (3αR,6R,6αR)-6-(4-((tert-butyldimethylsilyl)oxy)phenyl)-2,2-dimethyltetrahydro-4H cyclopenta[d][1,3]dioxol-4-one (222):/>

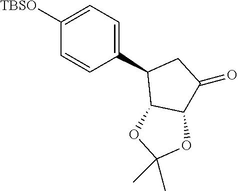 [SMILES: CC1(C)O[C@@H]2[C@@H](c3ccc(O[Si](C)(C)C(C)(C)C)cc3)CC(=O)[C@@H]2O1]

Following the general procedure H, compound 222 was obtained starting from (3αR,6αR)-2,2-dimethyl-3a,6a-dihydro-4H-cyclopenta[d][1,3]dioxol-4-one (0.50 g, 3.21 mmol), 209 (1.51 g, 4.48 mmol), [Cp*RhCl₂]₂ (0.16 g, 0.32 mmol), and KOH (0.03 g, 0.60 mmol) in dry degassed 1,4-dioxane (10.00 mL) and degassed water (0.60 mL) after column chromatography on silica (cyclohexane/EtOAc; 1-10%) as orange resin (1000 mg). R^{f}=0.16 (Cyclohexane/EtOAc 9:1). APCI: calcd. for C₂₀H₃₀O₄Si [M+H]⁺: 363.2 found 263.0, 304.9.

### (3αR,6R,6αR)-6-(3-chlorophenyl)-2,2-dimethyltetrahydro-4H-cyclopenta[d][1,3]dioxol-4-one (223)

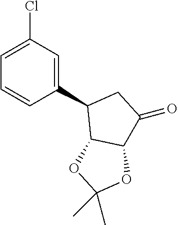 [SMILES: CC1(C)O[C@@H]2[C@@H](c3cccc(Cl)c3)CC(=O)[C@@H]2O1]

Following the general procedure H, compound 223 was obtained starting from (3αR,6αR)-2,2-dimethyl-3a,6a-dihydro-4H-cyclopenta[d][1,3]dioxol-4-one (0.61 g, 3.92 mmol), 2-(3-chlorophenyl)-4,4,5,5-tetramethyl-1,3,2-dioxaborolane (1.31 g, 5.51 mmol), [Cp*RhCl₂]₂ (0.19 g, 0.37 mmol), and KOH (0.04 g, 0.78 mmol) in dry degassed 1,4-dioxane (25.00 mL) and degassed water (0.70 mL) after column chromatography on silica (petrol ether/EtOAc; 0-12%) as yellowish solid (718 mg, 69%). R^{f}=0.14 (Petrolether/EtOAc 9:1). APCI: calcd. for C₁₄H15ClO₃ [M+H]⁺: 267.1 found 250.7 (—H₂O) ¹H NMR (400 MHZ, DMSO-d₆) δ 7.41-7.32 (m, 3H, o-H, m-H, p-H), 7.24-7.20 (m, 1H, o-H), 4.74 (dd, J=6.0, 3.1 Hz, 1H, H3′), 4.62-4.58 (m, 1H, H2′), 3.59-3.52 (m, 1H, H4′), 2.97-2.88 (m, 1H, H5A′), 2.59 (ddd, J=18.2, 7.0, 1.6 Hz, 1H, H5B′), 1.40 (s, 3H, CH₃, acetonide), 1.28 (s, 3H, CH₃, acetonide).

### (3αR,6R,6αR)-2,2-dimethyl-6-(3-(2-methyl-1,3-dioxolan-2-yl)phenyl) tetrahydro-4H-cyclopenta[d][1,3]dioxol-4-one (224)

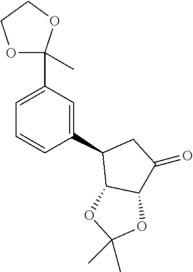 [SMILES: CC1(C)O[C@@H]2[C@@H](c3cccc(C4(C)OCCO4)c3)CC(=O)[C@@H]2O1]

Following the general procedure H, compound 224 was obtained starting from (3αR,6αR)-2,2-dimethyl-3a,6a-dihydro-4H-cyclopenta[d][1,3]dioxol-4-one (0.16 g, 1.02 mmol), compound 210 (0.43 g, 1.46 mmol), [Cp*RhCl₂]₂ (0.05 g, 0.10 mmol), and KOH (0.01 g, 0.23 mmol) in dry degassed 1,4-dioxane (6.50 mL) and degassed water (0.20 mL) after column chromatography on silica (cyclohexane/EtOAc; 1-50%) as yellow liquid (238 mg, 73%). R^{f}=0.21 (Petrolether/EtOAc 8:2). APCI: calcd. for C₁₈H₂₂O₅ [M+H]⁺: 319.2 found 318.8 ¹H NMR (400 MHZ, DMSO-d₆) δ 7.38-7.28 (m, 3H, o-H, p-H, m-H), 7.17 (dt, J=7.1, 1.8 Hz, 1H, o-H), 4.73 (dd, J=5.9, 2.8 Hz, 1H, H3′), 4.58-4.54 (m, 1H, H2′), 3.98 (td, J=6.3 Hz, 3.9 Hz, 2H, CH₂, ketal), 3.69 (td, J=5.9 Hz, 3.7 Hz, 2H, CH₂, ketal), 3.60-3.52 (m, 1H, H4′), 2.96 (dd, J=18.2 Hz, 8.8 Hz, 1H, H5′A), 2.60-2.52 (m, 1H, H5′B), 1.55 (s, 3H, CH₃, ketal), 1.41 (s, 3H, CH₃, acetonide), 1.29 (s, 3H, CH₃, acetonide).

### tert-butyl (4-((3ar,4r,6ar)-2,2-dimethyl-6-oxotetrahydro-4H-cyclopenta[d][1,3]dioxol-4-yl)phenyl)carbamate (225)

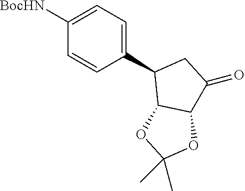 [SMILES: CC(C)(C)OC(=O)Nc1ccc([C@H]2CC(=O)[C@@H]3OC(C)(C)O[C@H]23)cc1]

570 mg (5.64 mmol; 1.00 eq) of (3ar,6ar)-2,2-dimethyl-3a,6a-dihydro-4H-cyclopenta[d][1,3]dioxol-4-one were dissolved in 50 ml of Dioxane and 5 mL of dist. H₂O. 2700 mg (8.46 mmol; 1.50 eq) of tert-butyl (4-(4,4,5,5-tetramethyl-1,3,2-dioxaborolan-2-yl)phenyl)carbamate, 63 mg (1.12 mmol; 0.20 eq) of KOH and 276 mg (0.56 mmol; 0.10 eq) of R^{h} ((COD) C1) 2 were added. The reaction mixture was heated under refluxation and inert gas atmosphere overnight. The solvent was removed under reduced pressure and the residue was purified by column chromatography (Cyclohexane/EtOAc 99:1% to 0:100%). The product was used without further characterization. TLC: R^{f}=0.52 (Cyclohexane/EtOAc 2:1)

### tert-butyl (3-((3ar,4r,6ar)-2,2-dimethyl-6-oxotetrahydro-4H-cyclopenta[d][1,3]dioxol-4-yl)phenyl)carbamate (226)

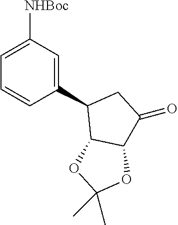 [SMILES: CC(C)(C)OC(=O)Nc1cccc([C@H]2CC(=O)[C@@H]3OC(C)(C)O[C@H]23)c1]

1044 mg (6.77 mmol; 1.00 eq) of (3ar,6ar)-2,2-dimethyl-3a,6a-dihydro-4H-cyclopenta[d][1,3]dioxol-4-one were dissolved in 50 ml of Dioxane and 5 ml of dist. H₂O. 2380 mg (7.45 mmol; 1.10 eq) of tert-butyl (3-(4,4,5,5-tetramethyl-1,3,2-dioxaborolan-2-yl)phenyl)carbamate, 77 mg (1.35 mmol; 0.20 eq) of KOH and 341 mg (0.68 mmol; 0.10 eq) of R^{h} ((COD) C1) 2 were added. The reaction mixture was heated under refluxation and inert gas atmosphere overnight. The solvent was removed under reduced pressure and the residue was purified by column chromatography (Cyclohexane/EtOAc 99:1% to 50:50%). The product (1100 mg; 3.16 mmol; 47%) was used without further characterization. TLC:
R^{f}=0.76 (Cyclohexane/EtOAc 1:1)

### (3αS,45,6R,6αR)-6-(3,5-dichlorophenyl)-2,2-dimethyltetrahydro-4H-cyclopenta[d][1,3]dioxol-4-ol (227)

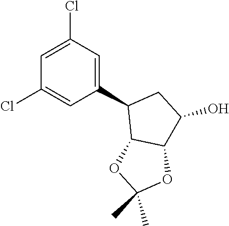

Following the general procedure for ketone reduction I, compound 227 was obtained starting from compound 213 (0.86 g, 2.82 mmol), NaBH₄ (0.16 g, 4.24 mmol) in dry MeOH (0.20 M, 14.10 mL) after column chromatography on silica (cyclohexane/EtOAc; 0-100%) as colorless resin (731 mg, 85%). R^{f}=0.44 (cyclohexane/EtOAc; 30%). ¹H NMR (400 MHZ, DMSO-d₆) δ7.47-7.45 (m, 1H, p-Ar), 7.29 (dd, J=1.9, 0.6 Hz, 2H, o-Ar), 4.65-4.57 (m, 1H, H3′, 4.59-4.52 (m, 1H, H2′), 4.51 (d, J=5.2 Hz, 1H, —OH), 3.96 (dq, J=7.3, 5.1 Hz, 1H, H1′), 3.25 (td, J=6.9, 2.9 Hz, 1H, H4′), 2.11 (dt, J=12.7, 7.4 Hz, 1H, H5′), 1.81 (dt, J=12.7, 5.5 Hz, 1H, H5′), 1.46 (s, 3H, CH₃, acetonide), 1.28 (s, 3H, CH₃, acetonide). APCI: calc. for C14H17Cl₂O3 [M+H]⁺: 303.05, found: 303.10/305.10.

### (3αS,4S,6R,6αR)-2,2-dimethyl-6-phenyltetrahydro-4H-cyclopenta[d][1,3]dioxol-4-ol (228)

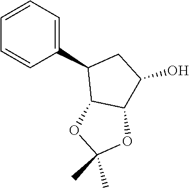

Following the general procedure for ketone reduction I, compound 228 was obtained starting from compound 214 (1.13 g, 4.82 mmol), NaBH₄ (0.28 g, 7.22 mmol) in dry MeOH (0.20 M, 24.10 mL) after column chromatography on silica (cyclohexane/EtOAc; 0-50%) as colorless resin (954 mg, 85%). R_{f}=0.22 (cyclohexane/EtOAc; 30%). ¹H NMR (400 MHZ, DMSO-d₆) δ7.33-7.28 (m, 2H, m-Ar), 7.22-7.17 (m, 3H, o,p-Ar), 4.60 (dd, J=6.3, 2.3 Hz, 1H, H3′), 4.57-4.49 (m, 1H, H2′), 4.45 (d, J=5.7 Hz, 1H, —OH), 4.04-3.95 (m, 1H, H1′), 3.19 (ddd, J=7.5, 5.1, 2.3 Hz, 1H, H4′), 2.10 (dt, J=12.5, 7.8 Hz, 1H, H5′), 1.79 (dt, J=12.5, 5.2 Hz, 1H, H5′), 1.46 (s, 3H, CH₃, acetonide), 1.27 (s, 3H, CH₃, acetonide). APCI: calc. for C₁₄H₁₉O₃ [M+H]⁺: 235.13, found: 235.20.

### ((3αS,45,6R,6αR)-2,2-dimethyl-6-(naphthalen-2-yl) tetrahydro-4H-cyclopenta[d][1,3]dioxol-4-ol (229)

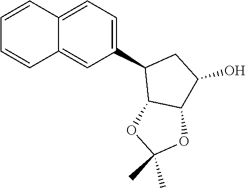

Following the general procedure for ketone reduction I, compound 229 was obtained starting from compound 215 (2.17 g, 7.60 mmol), NaBH₄ (0.44 g, 11.40 mmol) in dry MeOH (0.20 M, 38.00 mL) after column chromatography on silica (cyclohexane/EtOAc; 0-60%) as colorless resin (2251 mg, 7.92 mmol). R^{f}=0.85 (cyclohexane/EtOAc; 50%). APCI: calc. for C₁₈H21O₃ [M+H]⁺: 285.13, found: 285.10.

### (3αS,4S,6R,6αR)-2,2-dimethyl-6-(pyridin-4-yl) tetrahydro-4H-cyclopenta[d][1,3]dioxol-4-ol (230)

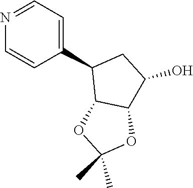

Following the general procedure for ketone reduction I, compound 230 was obtained starting from compound 216 (1.09 g, 4.63 mmol), NaBH₄ (0.27 g, 6.94 mmol) in dry MeOH (0.20 M, 23.10 mL) after column chromatography on silica (CH₂Cl₂/MeOH; 0-10%) as colorless resin (862 mg, 79%). R^{f}=0.22 (EtOAc; 100%). ¹H NMR (400 MHZ, DMSO-d₆) δ8.49-8.46 (m, 2H, H2, H6 pyridine), 7.25-7.22 (m, 2H, H3, H5 pyridine), 4.62 (dd, J=6.2, 2.4 Hz, 1H, H3′), 4.56 (d, J=5.6 Hz, 1H, —OH), 4.55-4.51 (m, 1H, H2′), 4.00-3.92 (m, 1H, H1′), 3.19 (ddd, J=7.6, 5.1, 2.4 Hz, 1H, H4′), 2.12 (dt, J=12.7, 7.9 Hz, 1H, H5′), 1.82 (dt, J=12.5, 5.2 Hz, 1H, H5′), 1.46 (s, 3H, CH₃, acetonide), 1.28 (s, 3H, CH₃, acetonide). APCI: calc. for C₁₃H₁₈NO₃ [M+H]⁺: 236.12, found: 236.30.

### (3αS,45,6S,6αR)-2,2-dimethyl-6-(thiophen-2-yl) tetrahydro-4H-cyclopenta[d][1,3]dioxol-4-ol (231)

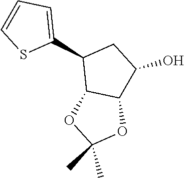

Following the general procedure for ketone reduction I, compound 231 was obtained starting from compound 217 (0.32 g, 0.79 mmol), NaBH₄ (0.05 g, 1.19 mmol) in dry MeOH (0.20 M, 4.00 mL) after column chromatography on silica (cyclohexane/EtOAc; 0-50%) as colorless resin (265 mg, 84%). R^{f}=0.72 (cyclohexane/EtOAc; 50%). ¹H NMR: (400 MHZ DMSO-d₆): δ 7.37 (dd, J=5.1, 1.2 Hz, 1H, H5-thiophene), 6.95 (dd, J=5.1, 3.5 Hz, 1H, H4 thiophene), 6.91 (dt, J=3.5, 1.2 Hz, 1H, H3-thiophene), 4.50-4.45 (m, 1H, H1′), 4.05-3.96 (m, 1H, H3′), 3.42-3.24 (m, 1H, H4′), 2.17-2.06 (m, 1H. H5′), 1.89 (m, 1H, H5′), 1.47-1.41 (m, 3H, CH₃), 1.27 (d, J=0.7 Hz, 3H, CH₃). APCI: calc. for C₁₂H₁₇O₃S [M+H]⁺: 241.08, found: 241.20.

### (3αS,45,6R,6αR)-2,2-dimethyl-6-(1//-pyrazol-5-yl) tetrahydro-4H-cyclopenta[d][1,3]dioxol-4-ol (232)

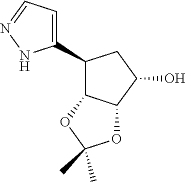

Following the general procedure for ketone reduction I, compound 232 was obtained starting from compound 218 (0.59 g, 1.81 mmol), NaBH₄ (0.10 g, 2.72 mmol) in dry MeOH (0.20 M, 9.10 mL) after column chromatography on silica (cyclohexane/EtOAc; 0-50%) as colorless resin (315 mg, 76%). R^{f}=0.44 (cyclohexane/EtOAc; 50%). ¹H NMR (400 MHZ, DMSO-d₆) δ 8.70 (s, 1H, H2), 8.07 (d, J=3.7 Hz, 1H, H6), 7.90 (dd, J=2.3, 0.6 Hz, 1H, H3 pyrazol), 7.56 (dd, J=1.8, 0.5 Hz, 1H, —NH pyrazol), 6.77 (d, J=3.7 Hz, 1H, H5), 6.32 (dd, J=2.3, 1.9 Hz, 1H, H4 pyrazol), 5.32-5.25 (m, 1H, H1′), 5.10 (dd, J=7.7, 5.6 Hz, 1H, H2′), 5.02 (dd, J=7.7, 5.4 Hz, 1H, H3′), 4.93-4.85 (m, 1H, H4′), 3.01 (q, J=12.2 Hz, 1H, H5′), 2.76-2.66 (m, 1H, H5′), 1.56 (s, 3H, CH₃, acetonide), 1.40 (s, 3H, CH₃, acetonide). APCI: calc. for C₁₁H₁₇N₂O₃ [M+H]⁺: 225.12, found: 225.00.

### (3αS,45,6R,6αR)-2,2-dimethyl-6-(quinolin-6-yl) tetrahydro-4H-cyclopenta[d][1,3]dioxol-4-ol (233)

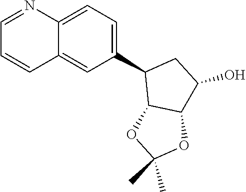

Following the general procedure for ketone reduction I, compound 233 was obtained starting from compound 219 (0.45 g, 1.57 mmol), NaBH₄ (0.09 g, 2.36 mmol) in dry MeOH (0.20 M, 7.90 mL) after column chromatography on silica (petrol ether/EtOAc; 0-100%) as colorless resin (370 mg, 1.30 mmol). R^{f}=0.09 (cyclohexane/EtOAc; 60%). APCI calcd.: 285.14; found: 285.90 [M+H]⁺. ¹H NMR (400 MHZ, DMSO-d₆) δ 8.85 (dd, J=4.2, 1.7 Hz, 1H, H2 quinoline), 8.35-8.30 (m, 1H, H4 quinoline), 7.97 (d, J=8.7 Hz, 1H, H8 quinoline), 7.78-7.76 (m, 1H, H5 quinoline), 7.64 (dd, J=8.8, 2.1 Hz, 1H, H7 quinoline), 7.51 (dd, J=8.3, 4.2 Hz, 1H, H3 quinoline), 4.73 (dd, J=6.2, 2.3 Hz, 1H, H3′), 4.62-4.57 (m, 1H, H2′), 4.56-4.49 (m, 1H, —CHOH), 4.10-4.05 (m, 1H, H1′), 3.43-3.37 (m, 1H, H4′), 2.19 (dt, J=12.6, 7.9 Hz, 1H, H5′), 1.99-1.89 (m, 1H, H5′), 1.49 (s, 3H, CH₃, acetonide), 1.30 (s, 3H, CH₃, acetonide). Note: product contained 30% of the unfavored alcohol.

### tert-butyl (3-((3αR,4R,6S,6αS)-6-hydroxy-2,2-dimethyltetrahydro-4H-cyclopenta[d][1,3]dioxol-4-yl)benzyl)carbamate (234)

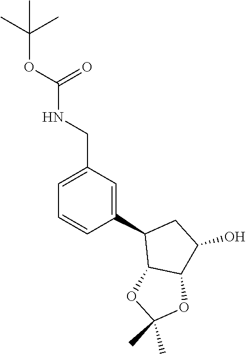

Following the general procedure for ketone reduction I, compound 234 was obtained starting from compound 220 (3.15 g, 8.63 mmol), NaBH₄ (0.50 g, 12.94 mmol) in dry MeOH (0.20 M, 43.10 mL) after column chromatography on silica (n-heptane/EtOAc; 0-50%) as white solid (2311 mg, 74%). R^{f}=0.32 (n-heptane/EtOAc; 50%). ¹H NMR (400 MHZ, DMSO-d₆) δ7.38 (t, J=6.0 Hz, 1H, —NH), 7.28-7.21 (m, 1H, m-Ar), 7.10-7.03 (m, 3H, o,p-Ar), 4.58 (dd, J=6.1, 2.1 Hz, 1H, H3′), 4.54-4.49 (m, 1H, H2′), 4.11 (d, J=6.2 Hz, 1H, H1′), 4.03-3.97 (m, 2H, —NHCH₂—), 3.20-3.13 (m, 1H, H4′), 2.09 (dt, J=12.5, 7.9 Hz, 1H, H5′), 1.80 (dt, J=12.3, 5.1 Hz, 1H, H5′), 1.47 (s, 3H, CH₃, acetonide), 1.41 (s, 9H, —NCO₂C(CH₃)₃), 1.28 (s, 3H, CH₃, acetonide). APCI: calc. for C20H30NO₅ [M+H]⁺: 364.20, found: 623.10 [2M+H, —Boc]⁺.

### tert-butyl 2-((3αR,4R,65,6αS)-6-hydroxy-2,2-dimethyltetrahydro-4H-cyclopenta[d][1,3]dioxol-4-yl) pyrrolidine-1-carboxylate (235)

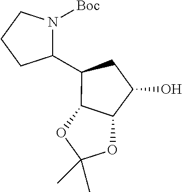 [SMILES: CC(C)(C)OC(=O)N1CCCC1[C@H]1C[C@H](O)[C@@H]2OC(C)(C)O[C@@H]21]

Following the general procedure for ketone reduction I, compound 235 was obtained starting from compound 205 (0.35 g, 1.07 mmol), NaBH₄ (0.06 g, 1.60 mmol) in dry MeOH (0.20 M, 5.30 mL) after column chromatography on silica (cyclohexane/EtOAc; 0-40%) as a colorless resin (260 mg, 75%). R^{f}=0.15 (petrol ether/EtOAc; 30%). ¹H NMR (400 MHZ, CDCl₃) δ4.66-4.30 (m, 4H, H2, H3′), 4.21-4.03 (m, 2H, H1′), 3.86-3.59 (m, 2H, —NC H—), 3.58-3.28 (m, 3H, —NC H₂CH₂CH₂—), 3.25 (ddd, J=11.7, 8.0, 3.7 Hz, 1H, —NC H₂CH₂CH₂—), 2.41 (s, 2H, —OH), 2.20-2.09 (m, 1H, H4′), 2.03-1.60 (m, 13H, H4, H5′, —NC H₂CH₂CH₂—), 1.49 (s, 3H, CH₃, acetonide), 1.49-1.42 (m, 21H, —NCO₂C(CH₃)₃, CH₃, acetonide), 1.34 (s, 3H, CH₃, acetonide), 1.32 (s, 3H, CH₃, acetonide). HRMS calcd.: 327.20; found: 350.19 [M+Na]⁺.

### tert-butyl 2-((3αR,4R,65,6αS)-6-hydroxy-2,2-dimethyltetrahydro-4/cyclopenta[d][1,3]dioxol-4-yl) piperidine-1-carboxylate (236)

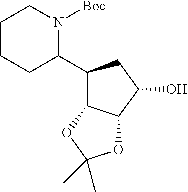 [SMILES: CC(C)(C)OC(=O)N1CCCCC1[C@H]1C[C@H](O)[C@@H]2OC(C)(C)O[C@@H]21]

Following the general procedure for ketone reduction I, compound 236 was obtained starting from compound 206 (0.40 g, 1.17 mmol), NaBH₄ (0.07 g, 1.75 mmol) in dry MeOH (0.20 M, 5.80 mL) after column chromatography on silica (cyclohexane/EtOAc; 0-40%) as a colorless resin (300 mg, 75%). R^{f}=0.26 (petrol ether/EtOAc; 30%). ¹H NMR (400 MHZ, Chloroform-d) 84.51 (dd, J=6.6, 5.2 Hz, 1H, H2′), 4.27 (dd, J=6.7, 3.1 Hz, 1H, H3′), 4.17-3.72 (m, 3H, H1′, H6 piperidine), 2.76-2.58 (m, 2H, H4′, H2 piperidine), 2.54-2.36 (m, 1H, H2 piperidine), 1.85-1.74 (m, 2H, H5′, H5 piperidine), 1.70-1.53 (m, 5H, H3, H4, H5 piperidine), 1.51 (s, 3H, CH₃, acetonide), 1.49 (s, 3H (1.4), CH₃, acetonide), 1.47-1.41 (m, 9H, —NCO₂C(CH₃)₃), 1.34 (s, 3H, CH₃, acetonide), 1.29 (s, 3H (1.7), CH₃, acetonide).

### (3αS,4S,6R,6αR)-6-(3-((tert-butyldimethylsilyl)oxy)phenyl)-2,2-dimethyltetrahydro-4H-cyclopenta[d][1,3]dioxol-4-ol (237)

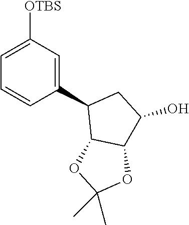 [SMILES: CC1(C)O[C@@H]2[C@H](O1)[C@@H](c1cccc(O[Si](C)(C)C(C)(C)C)c1)C[C@@H]2O]

Following the general procedure for ketone reduction I, compound 237 was obtained starting from compound 221 (0.84 g, 2.33 mmol), NaBH₄ (0.18 g, 4.68 mmol) in dry MeOH (0.20 M, 6.00 mL) after column chromatography on silica (cyclohexane/EtOAc; 1-21%) as a colorless resin (506 mg, 60%). R^{f}=0.29 (Cyclohexane/EtOAc 8:2). APCI: calcd. for C₂₀H32O₄Si [M+H]⁺: 365.2 found 289.0, 363.9, 364.9. ¹H NMR (400 MHZ, DMSO-d₆) δ 7.18 (t, J=7.8 Hz, 1H, m-H), 6.78 (d, J=7.7 Hz, 1H, o-H), 6.70-6.67 (m, 1H, o-H), 6.64-6.63 (m, 1H, p-H), 4.58-4.54 (m, 1H, H3′), 4.52-4.50 (m, 1H, H2′), 4.47 (d, J=5.6 Hz, 1H, OH), 3.98 (dq, J=8.1, 5.3 Hz, 1H, H1′), 3.16-3.12 (m, 1H, H4′), 2.08 (dt, J=12.6, 7.9 Hz, 1H, H5′A), 1.77 (dt, J=12.5, 5.2 Hz, 1H, H5′B), 1.46 (s, 3H, CH₃. acetonide), 1.28 (s, 3H, CH₃, acetonide), 0.95 (s, 9H, C(CH₃)₃), 0.18 (s, 6H, Si (CH₃) 2)

### (3αS,4S,6R,6αR)-6-(4-((tert-butyldimethylsilyl)oxy)phenyl)-2,2-dimethyltetrahydro-4H cyclopenta[d][1,3]dioxol-4-ol (238)

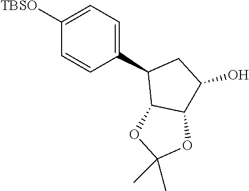 [SMILES: CC1(C)O[C@@H]2[C@H](O1)[C@@H](c1ccc(O[Si](C)(C)C(C)(C)C)cc1)C[C@@H]2O]

Following the general procedure for ketone reduction I, compound 238 was obtained starting from compound 222 (0.99 g, 2.71 mmol), NaBH₄ (0.22 g, 5.74 mmol) in dry MeOH (0.20 M, 7.00 mL) after column chromatography on silica (CH₂Cl₂/MeOH; 0.1-1%) as a colorless resin (612 mg). R^{f}=0.32 (Petrolether/EtOAc 8:2). APCI: calcd. for C₂₀H32O₄Si [M+H]⁺: 365.2 found 289.0, 364.0, 365.0. ¹H NMR (400 MHZ, DMSO-d₆) δ 7.08-7.03 (m, 2H, 2 x o-H), 6.79-6.75 (m, 2H, 2 x m-H), 4.56-4.52 (m, 1H, H3′), 4.52-4.48 (m, 1H, H2′), 4.42 (d, J=5.6, 1H, OH), 4.00-3.94 (m, 1H, H1′), 3.14-3.10 (m, 1H, H4), 2.05 (dt, J=12.6 Hz, 7.8 Hz, 1H, H5′A), 1.80-1.72 (m, 1H, H5′B), 1.45 (s, 3H, CH₃, acetonide), 1.26 (s, 3H, CH₃, acetonide), 0.94 (s, 9H, C(CH₃)₃), 0.16 (s, 6H, Si (CH₃) 2). Unknown impurities from cyclohexane (used for flash chromatography) were detected at 1.5 ppm, 2.3 ppm and 3.6 ppm by NMR

### (3αS,45,6R,6αR)-6-(3-chlorophenyl)-2,2-dimethyltetrahydro-4H-cyclopenta[d][1,3]dioxol-4-ol (239)

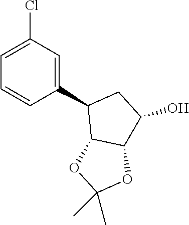 [SMILES: CC1(C)O[C@@H]2[C@H](O1)[C@@H](c1cccc(Cl)c1)C[C@@H]2O]

Following the general procedure for ketone reduction I, compound 239 was obtained starting from compound 223 (0.71 g, 2.65 mmol), NaBH₄ (0.21 g, 5.42 mmol) in dry MeOH (0.20 M, 7.00 mL) after column chromatography on silica (CH₂Cl₂/MeOH; 0.1-1%) as a colorless resin (181 mg, 25%). R_{f}=0.21 (Petrolether/EtOAc 8:2). APCI: calcd. for C₁₄H₁₇ClO₃ [M+H]⁺: 269.1 found 253 (—H₂O) ¹H NMR (400 MHZ, DMSO-d₆) δ 7.37-7.32 (m, 1H, m-H), 7.30-7.26 (m, 2H, p-H, o-H), 7.19-7.16 (m, 1H, o-H), 4.62-4.58 (m, 1H, H3′), 4.57-4.52 (m, 1H, H2′), 4.49 (d, J=5.4 Hz, 1H, OH), 3.98 (dq, J=7.7, 5.2 Hz, 1H, H1′), 3.25-3.20 (m, 1H, H4′), 2.11 (dt, J=12.7, 7.6 Hz, 1H, H5A′), 1.80 (dt, J=12.7, 5.5 Hz, 1H, H5B′), 1.47 (s, 3H, CH₃, acetonide), 1.28 (s, 3H, CH₃, acetonide).

### (3αS,45,6R,6αR)-2,2-dimethyl-6-(3-(2-methyl-1,3-dioxolan-2-yl)phenyl) tetrahydro-4H-cyclopenta[d][1,3]dioxol-4-ol (240)

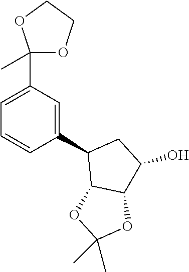 [SMILES: CC1(C)O[C@@H]2[C@H](O1)[C@@H](c1cccc(C3(C)OCCO3)c1)C[C@@H]2O]

Following the general procedure for ketone reduction I, compound 240 was obtained starting from compound 224 (0.23 g, 0.72 mmol), NaBH₄ (0.04 g, 1.08 mmol) in dry MeOH (0.20 M, 7.00 mL) after column chromatography on silica (cyclohexane/EtOAc; 5-50%) as a colorless resin (200 mg). APCI: calcd. for C₁₈H24O₅ [M+H]⁺: 321.2 found 320.8/366.7.

### tert-butyl (4-((3αR,4R,65,6αS)-6-hydroxy-2,2-dimethyltetrahydro-4H-cyclopenta[d][1,3]dioxol-4-yl)phenyl)carbamate (241)

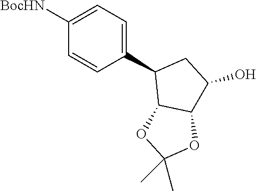 [SMILES: CC(C)(C)OC(=O)Nc1ccc([C@H]2C[C@H](O)[C@@H]3OC(C)(C)O[C@@H]32)cc1]

3330 mg (9.59 mmol; 1.00 eq) of tert-butyl (4-((3ar,4r,6ar)-2,2-dimethyl-6-oxotetrahydro-4H-cyclopenta[d][1,3]dioxol-4-yl)phenyl)carbamate were dissolved in 40 ml of MeOH. After cooling with an ice-bath to 0° C., 545 mg (14.39 mmol; 1.50 eq) of NaBH₄ were added portionwise. The reaction mixture was warmed to RT and thereby continuously stirred for 4 h. The solvent was then removed under reduced pressure and the resulting residue was purified by flash chromatography (Cyclohexane/EtOAc 99:1% to 50:50%) to give the desired compound (300 mg; 0.86 mmol; 9%) in the form of a colorless liquid. TLC: R^{f}=0.63 (Cyclohexane/EtOAc 1:1). 1H-NMR: (400 MHZ, DMSO-d₆) δ=9.26 (s, 1H), 7.41-7.33 (m, 2H), 7.09-7.01 (m, 2H), 4.57-4.53 (m, 1H), 4.51-4.48 (m, 1H), 4.42 (d, J=5.8 Hz, 1H), 4.00-3.93 (m, 1H), 3.13-3.07 (m, 1H), 2.08-2.01 (m, 1H), 1.79-1.72 (m, 1H), 1.46 (s, 9H), 1.45 (s, 3H), 1.27 (s, 3H) ppm.

### tert-butyl (3-((3αR,4R,6S,6αS)-6-hydroxy-2,2-dimethyltetrahydro-4H-cyclopenta[d][1,3]dioxol-4-yl)phenyl)carbamate (242)

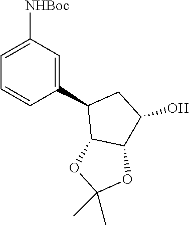 [SMILES: CC(C)(C)OC(=O)Nc1cccc([C@H]2C[C@H](O)[C@@H]3OC(C)(C)O[C@@H]32)c1]

1100 mg (3.16 mmol; 1.00 eq) of tert-butyl (3-((3ar,4r,6ar)-2,2-dimethyl-6-oxotetrahydro-4H cyclopenta[d][1,3]dioxol-4-yl)phenyl)carbamate were dissolved in 40 ml of MeOH. After cooling with an ice-bath to 0° C., 179 mg (4.74 mmol; 1.50 eq) of NaBH₄ were added portionwise. The reaction mixture was warmed to RT and thereby continuously stirred for 2 h. The solvent was then removed under reduced pressure and the resulting residue was purified by flash chromatography (Cyclohexane/EtOAc 99:1% to 75:25%) to give the desired compound (950 mg; 2.72 mmol; 86%) in the form of a colorless liquid, which was used without further characterization. TLC: R^{f}=0.48 (Cyclohexane/EtOAc 1:1)

### tert-butyl (4-((3αR,4R,6R,6αS)-6-(4-chloro-7H-pyrrolo[2,3-d]pyrimidin-7-yl)-2,2-dimethyltetrahydro-4H-cyclopenta[d][1,3]dioxol-4-yl)phenyl)carbamate (243)

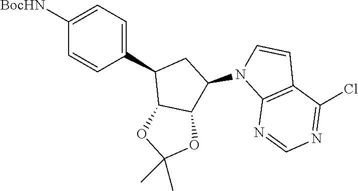 [SMILES: CC(C)(C)OC(=O)Nc1ccc([C@H]2C[C@@H](n3ccc4c(Cl)ncnc43)[C@@H]3OC(C)(C)O[C@@H]32)cc1]

300 mg (0.86 mmol; 1.00 eq) of tert-butyl (4-((3ar,4r,6as)-6-hydroxy-2,2-dimethyltetrahydro-4H cyclopenta[d][1,3]dioxol-4-yl)phenyl)carbamate were dissolved in 30 ml of Toluene under inert gas atmosphere. At 0° C., 450 mg (1.72 mmol; 2.00 eq) of PPh₃ were added. 10 minutes later 400 mg (1.72 mmol; 2.00 eq) of Di-tert-butyl-azodicarboxylate were attached. The reaction solution was stirred for further 10 minutes before 170 mg (1.12 mmol; 1.30 eq) of 4-chloro-7H-pyrrolo[2,3-d]pyrimidine were added and the whole was heated to 80° C. overnight. The solvent was removed under reduced pressure and the crude was purified by column chromatography (Petrolether/EtOAc 99:1% to 60:40%; R^{f}=0.83 in Petrolether/EtOAc 1:1). The product was directly used without further characterization. Therefore 20 ml of Dioxane and 20 ml of a 25% aqueous solution of NH₃ were added. The reaction solution was heated to 100° C. in a sealed tube overnight. The solvent was then removed under reduced pressure and the resulting residue was adsorbed on silica and purified by flash chromatography (DCM/MeOH 99:1% to 90:10%). 260 mg (0.56 mmol; 65% over 2 steps) of the title compound were obtained in the form of a colorless foam. TLC: R^{f}=0.29 (DCM/MeOH 10:1). APCI-MS (+) m/z for C₂₅H₂₉ClN₄O₄: calc.: 484.98; found: 485.0 and 487.0. APCI-MS (+) m/z for C₂₅H₃₁N₅O₄: calc.: 465.55; found: 466.0 and 467.0.

### 4-chloro-7-((3αS,4R,6R,6αR)-6-(3,5-dichlorophenyl)-2,2-dimethyltetrahydro-4H-cyclopenta[d][1,3]dioxol-4-yl)-7H-pyrrolo[2,3-d]pyrimidine (244)

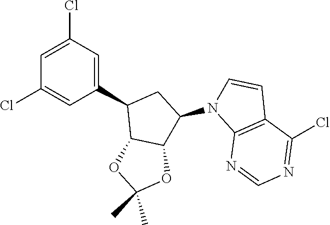

Following the general procedure for substitution/esterification J, compound 244 was obtained from first esterification of the alcohol 227 (0.36 g, 0.81 mmol) with Tf₂O (2.00 eq.), dry pyridine (3.00 eq.) in CH₂Cl₂ (0.10 M) followed by substitution with NaH (60%, 0.07 g, 1.62 mmol), 6-chloro-7-deazapurine (0.15 g, 0.97 mmol) in dry DMF (0.08 M, 10.10 mL). After column chromatography on silica (petrol ether/EtOAc; 0-50%) as white foam (202 mg, 57%). ¹H NMR (400 MHZ, DMSO-d₆) δ8.68 (s, 1H, H2), 8.14 (d, J=3.7 Hz, 1H, H6), 7.53-7.50 (m, 3H, o, p-Ar), 6.79 (d, J=3.7 Hz, 1H, H5), 5.36-5.25 (m, 1H, H1′), 4.94 (dd, J=7.5, 5.3 Hz, 1H, H2′), 4.79 (t, J=7.2 Hz, 1H, H3′), 3.41-3.34 (m, 1H, H4′), 2.63-2.53 (m, 2H, H5′), 1.54 (s, 3H, CH₃, acetonide), 1.23 (s, 3H, CH₃, acetonide).

### 5-bromo-4-chloro-7-((3αS,4R,6R,6αR)-6-(3,5-dichlorophenyl)-2,2-dimethyltetrahydro-4H-cyclopenta[d][1,3]dioxol-4-yl)-7H-pyrrolo[2,3-a]pyrimidine (245)

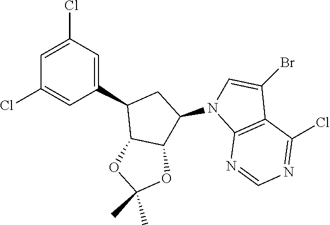

Following the general procedure for substitution/esterification J, compound 245 was obtained from first esterification of the alcohol 227 (0.65 g, 1.48 mmol) with Tf₂O (2.00 eq.), dry pyridine (3.00 eq.) in CH₂Cl₂ (0.10 M) followed by substitution with NaH (60%, 0.12 g, 2.96 mmol), 6-chloro-7-deazapurine (0.42 g, 1.77 mmol) in dry DMF (0.08 M, 18.50 mL). After column chromatography on silica (petrol ether/EtOAc; 0-40%) as white foam (240 mg, 31%). ¹H NMR (400 MHZ, DMSO-d₆) δ8.72 (s, 1H, H2 adenine), 8.44 (s, 1H, H6 adenine), 7.54-7.50 (m, 3H, o,p-Ar), 5.33 (ddd,)=11.8, 7.4, 5.0 Hz, 1H, H1′), 4.91 (dd, J=7.6, 5.1 Hz, 1H, H2′), 4.75 (t, J=7.2 Hz, 1H, H3′), 3.40-3.32 (m, 1H, H4′under water peak), 2.62-2.52 (m, 2H, H5′), 1.54 (s, 3H, CH₃, acetonide), 1.22 (s, 3H, CH₃, acetonide).

### 4-chloro-7-((3αS,4R,6R,6αR)-2,2-dimethyl-6-phenyltetrahydro-4//-cyclopenta[d][1,3]dioxol-4-yl)-7H-pyrrolo[2,3-d]pyrimidine (246)

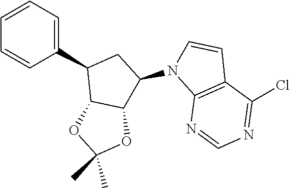

Following the general procedure for substitution/esterification J, compound 246 was obtained from first compound 228 (0.31 g, 0.72 mmol) with Tf₂O (2.00 eq.), dry pyridine (3.00 eq.) in CH₂Cl₂ (0.10 M) followed by substitution with NaH (60%, 0.06 g, 1.45 mmol), 6-chloro-7-deazapurine (0.14 g, 0.87 mmol) in dry DMF (0.08 M, 9.00 mL) after column chromatography on silica (cyclohexane/EtOAc; 0-50%) as white foam (189 mg, 59%). R^{f}=0.59 (cyclohexane/EtOAc; 40%). ¹H NMR (400 MHZ, DMSO-d₆) δ 8.68 (s, 1H, H2), 8.12 (d, J=3.7 Hz, 1H, H6), 7.43-7.33 (m, 4H, o,m-Ar), 7.29-7.24 (m, 1H, p-Ar), 6.77 (d, J=3.6 Hz, 1H, H5), 5.29 (ddd, J=11.3, 7.6, 5.5 Hz, 1H, H1′), 5.00 (dd, J=7.6, 5.5 Hz, 1H, H2′), 4.78 (t, J=7.2 Hz, 1H, H3′), 3.34-3.26 (m, 1H, H4′), 2.64-2.54 (m, 2H, H5′), 1.54 (s, 3H, CH₃, acetonide), 1.23 (s, 3H, CH₃, acetonide).

### 5-bromo-4-chloro-7-((3αS,4R,6R,6αR)-2,2-dimethyl-6-phenyltetrahydro-4H cyclopenta[d][1,3]dioxol-4-yl)-7H-pyrrolo[2,3-a]pyrimidine (247)

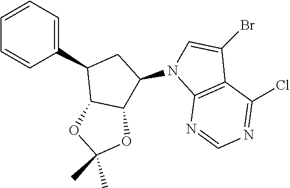

Following the general procedure K for Mitsunobu-type glycosylation, compound 247 was obtained starting from compound 228 (0.62 g, 2.63 mmol), 6-chloro-7-deazapurine (0.86 g, 3.68 mmol), PPh₃ (1.39 g, 5.26 mmol), and DIAD (0.94 mL, 4.73 mmol) in dry THF (0.10 M, 26.30 mL) after column chromatography on silica (petrol ether/EtOAc; 0-40%) as white foam (673 mg, 57%). R^{f}=0.60 (cyclohexane/EtOAc; 30%). ¹H NMR (400 MHZ, DMSO-d₆) δ8.72 (s, 1H, H2), 8.43 (s, 1H, H5), 7.43-7.33 (m, 4H, o, m-Ar), 7.30-7.22 (m, 1H, p-Ar), 5.32 (ddd, J=11.3, 7.6, 5.3 Hz, 1H, H1′), 4.96 (dd, J=7.6, 5.3 Hz, 1H, H2′), 4.75 (t, J=7.2 Hz, 1H, H3′), 3.32-3.25 (m, 1H, H4′), 2.63-2.51 (m, 2H, H5′), 1.53 (s, 3H, CH₃, acetonide), 1.22 (s, 3H, CH₃, acetonide).

### 4-chloro-7-((3αS,4R,6R,6αR)-2,2-dimethyl-6-(naphthalen-2-yl) tetrahydro-4H-cyclopenta[d][1.3]dioxol-4-yl)-7H-pyrrolo [2,3-d]pyrimidine (248)

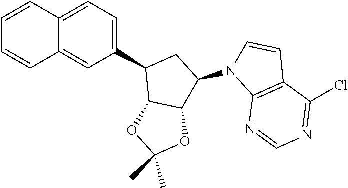

Following the general procedure K for Mitsunobu-type glycosylation, compound 248 was obtained starting from compound 229 (0.20 g, 0.70 mmol), 6-chloro-7-deazapurine (0.15 g, 0.98 mmol), PPh₃ (0.37 g, 1.39 mmol), and DIAD (0.25 mL, 1.25 mmol) in dry THF (0.10 M, 7.00 mL) after column chromatography on silica (petrol ether/EtOAc; 0-50%) as white foam (210 mg, 0.50 mmol). R^{f}=0.71 (cyclohexane/EtOAc; 50%). ¹H NMR (400 MHZ, DMSO-d₆) δ8.70 (s, 1H, H2 adenine), 8.17 (d, J=3.7 Hz, 1H, H6 adenine), 7.96-7.85 (m, 4H, H1, H4, H5, H8 naphthyl), 7.62 (dd, J=8.5, 1.8 Hz, 1H, H3 naphthyl), 7.54-7.45 (m, 2H, H6, H7 naphthyl), 6.79 (d, J=3.7 Hz, 1H, H5 adenine), 5.36 (dt, J=12.1, 6.3 Hz, 1H, H1′), 5.05 (dd, J=7.5, 5.5 Hz, 1H, H2′), 4.94-4.85 (m, 1H, H3′), 3.51 (dt, J=13.0, 6.4 Hz, 1H, H4′), 2.79-2.62 (m, 2H, H5′), 1.58 (s, 3H, CH₃, acetonide), 1.25 (s, 3H, CH₃, acetonide).

### 2,4-dichloro-7-((3αS,4R,6R,6αR)-2,2-dimethyl-6-(naphthalen-2-yl) tetrahydro-4H-cyclopenta[d][1.3]dioxol-4-yl)-7H-pyrrolo [2,3-d]pyrimidine (249)

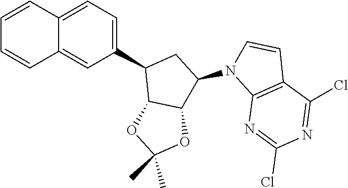

Following the general procedure K for Mitsunobu-type glycosylation, compound 249 was obtained starting from compound 229 (0.44 g, 1.53 mmol), 6-chloro-7-deazapurine (0.41 g, 2.14 mmol), PPh₃ (0.81 g, 3.10 mmol), and DIAD (0.54 mL, 2.75 mmol) in dry THF (0.10 M, 15.20 mL) after column chromatography on silica (petrol ether/EtOAc; 0-50%) as white foam (268 mg, 39%). ¹H NMR (400 MHZ, DMSO-d₆) δ7.93-7.84 (m, 4H, H4, H5, H8 naphthyl, H6 adenine), 7.83-7.81 (m, 1H, H1 naphthyl), 7.54-7.45 (m, 4H, H3, H6, H7 naphthyl, H5 adenine), 4.83-4.75 (m, 2H, H2′, H3′), 4.38-4.31 (m, 1H, H1′), 3.38-3.30 (m, 1H, H4′ under water peak), 2.75 (dt, J=12.7, 6.3 Hz, 1H, H5′), 2.26 (td, J=12.4, 11.0 Hz, 1H, H5′), 1.52 (s, 3H, CH₃, acetonide), 1.28 (s, 3H, CH₃, acetonide).

### 4-chloro-7-((3αS,4R,6R,6αR)-2,2-dimethyl-6-(pyridin-4-yl) tetrahydro-4H-cyclopenta[d][1,3]dioxol-4-yl)-7H-pyrrolo[2,3-d]pyrimidine (250)

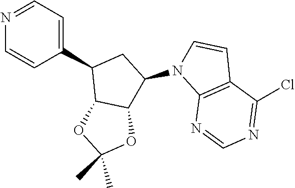

Following the general procedure K for Mitsunobu-type glycosylation, compound 250 was obtained starting from compound 230 (0.20 g, 0.84 mmol), 6-chloro-7-deazapurine (0.19 g, 1.18 mmol), PPh₃ (0.45 g, 1.68 mmol), and DIAD (0.30 mL, 1.52 mmol) in dry THF (0.15 M, 5.60 mL) after column chromatography on silica (petrol ether/EtOAc; 0-100%) as white foam (359 mg, 0.97 mmol). R^{f}=0.78 (cyclohexane/EtOAc; 50%). ¹H NMR (400 MHZ, DMSO-d₆) δ8.68 (s, 1H, H2 adenine), 8.56-8.52 (m, 2H, H2, H6 pyridine), 8.11 (d, J=3.7 Hz, 1H, H6 adenine), 7.45-7.41 (m, 2H, H3, H5 pyridine), 6.78 (d, J=3.7 Hz, 1H, H5), 5.39-5.27 (m, 1H, H1′), 4.99 (dd, J=7.5, 5.3 Hz, 1H, H2′), 4.82 (t, J=7.2 Hz, 1H, H3′), 3.39-3.34 (m, 1H, H4′), 2.65-2.57 (m, 2H, H5′), 1.55 (s, 3H, CH₃, acetonide), 1.24 (s, 3H, CH₃, acetonide).

### 4-chloro-7-((3αS,4R,6S,6αR)-2,2-dimethyl-6-(thiophen-2-yl) tetrahydro-4H-cyclopenta[d][1,3]dioxol-4-yl)-7H-pyrrolo[2,3-a]pyrimidine (251)

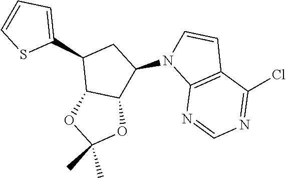

Following the general procedure K for Mitsunobu-type glycosylation, compound 251 was obtained starting from compound 231 (0.25 g, 1.03 mmol), 6-chloro-7-deazapurine (0.34 g, 1.44 mmol), PPh₃ (0.55 g, 2.06 mmol), and DIAD (0.37 mL, 1.85 mmol) in dry THF (0.25 M, 4.10 mL) after column chromatography on silica (petrol ether/EtOAc; 0-50%) as colorless resin (197 mg, 51%). R^{f}=0.73 (cyclohexane/EtOAc; 50%). ¹H NMR (400 MHZ, DMSO-d₆) δ8.68 (s, 1H, H2), 8.08 (d, J=3.7 Hz, 1H, H6), 7.42 (dd,)=4.8, 1.5 Hz, 1H, H5 thiophene), 7.04-6.99 (m, 2H, H3, H4 thiophene), 6.75 (d, J=3.6 Hz, 1H, H5), 5.28 (ddd, J=10.7, 8.1, 5.2 Hz, 1H, H1′), 5.00 (dd, J=7.5, 5.2 Hz, 1H, H2′), 4.77 (t, J=7.2 Hz, 1H, H3′), 3.56 (dt, J=11.3, 7.3 Hz, 1H, H4′), 2.68-2.59 (m, 2H, H5′), 1.54 (s, 3H, CH₃, acetonide), 1.24 (s, 3H, CH₃, acetonide).

### 4-chloro-7-((3αS,4R,6R,6αR)-2,2-dimethyl-6-(1H-pyrazol-5-yl) tetrahydro-4H-cyclopenta[d][1,3]dioxol-4-yl)-7H-pyrrolo[2,3-a]pyrimidine (252)

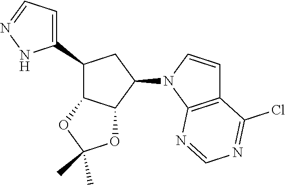

Following the general procedure K for Mitsunobu-type glycosylation, compound 252 was obtained starting from compound 232 (0.26 g, 0.79 mmol), 6-chloro-7-deazapurine (0.31 g, 1.11 mmol), PPh₃ (0.42 g, 1.59 mmol), and DIAD (0.29 mL, 1.43 mmol) in dry THF (0.15 M, 5.30 mL) after column chromatography on silica (petrol ether/EtOAc; 0-50%) as white foam (267 mg, 0.58 mmol). R_{f}=0.42 (petrol ether/EtOAc; 50%). ¹H NMR (400 MHZ, DMSO-d₆) δ 8.70 (s, 1H, H2), 8.07 (d, J=3.7 Hz, 1H, H6), 7.90 (dd, J=2.3, 0.6 Hz, 1H, H3 pyrazol), 7.56 (dd, J=1.8, 0.5 Hz, 1H, —NH pyrazol), 6.77 (d, J=3.7 Hz, 1H, H5), 6.32 (dd, J=2.3, 1.9 Hz, 1H, H4 pyrazol), 5.32-5.25 (m, 1H, H1′), 5.10 (dd, J=7.7, 5.6 Hz, 1H, H2′), 5.02 (dd, J=7.7, 5.4 Hz, 1H, H3′), 4.93-4.85 (m, 1H, H4′), 3.01 (q, J=12.2 Hz, 1H, H5′), 2.76-2.66 (m, 1H, H5′), 1.56 (s, 3H, CH₃, acetonide), 1.40 (s, 3H, CH₃, acetonide).

### 6-((3αR,4R,6R,6αS)-6-(2,4-dichloro-7H-pyrrolo[2,3-a]pyrimidin-7-yl)-2,2-dimethyltetrahydro-4H-cyclopenta[d][1,3]dioxol-4-yl) quinoline (253)

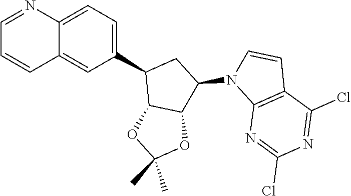

Following the general procedure K for Mitsunobu-type glycosylation, compound 253 was obtained starting from compound 233 (0.25 g, 0.87 mmol), 6-chloro-7-deazapurine (0.23 g, 1.21 mmol), PPh₃ (0.46 g, 1.74 mmol), and DIAD (0.32 mL, 1.56 mmol) in dry THF (0.15 M, 5.80 mL) after column chromatography on silica (petrol ether/EtOAc; 0-40%) as white foam (100 mg, 25%). ¹H NMR (400 MHZ, DMSO-d₆) δ 8.90-8.86 (m, 1H, H2 quinoline), 8.35 (d, J=8.6 Hz, 1H. H4 quinoline), 8.21 (d, J=3.7 Hz, 1H, H6 adenine), 8.05 (d, J=8.8 Hz, 1H, H8 quinoline), 7.94 (d, J=1.8 Hz, 1H, H5 quinoline), 7.87 (dd, J=8.8, 2.0 Hz, 1H, H7 quinoline), 7.58-7.51 (m, 1H, H3 quinoline), 6.87 (d, J=3.7 Hz, 1H, H5, adenine), 5.34-5.25 (m, 1H, H1′), 5.03-4.99 (m, 1H, H2′), 4.92-4.87 (m, 1H, H3′), 3.62-3.53 (m, 1H, H4′), 2.72-2.64 (m, 2H, H5′), 1.59 (s, 3H, CH₃, acetonide), 1.26 (s, 3H, CH₃, acetonide).

### tert-butyl (3-((3αR,4R,6R,6αS)-6-(5-bromo-2,4-dichloro-7H-pyrrolo[2,3-a]pyrimidin-7-yl)-2,2-dimethyltetrahydro-4H-cyclopenta[d][1 3]dioxol-4-yl)benzyl)carbamate (254)

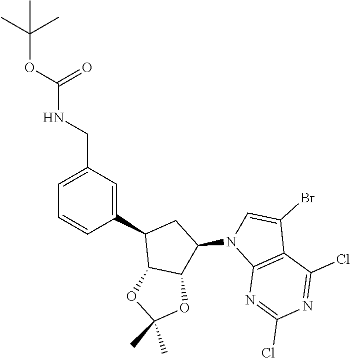

Following the general procedure L for Mitsunobu-type glycosylation, compound 254 was obtained starting from 5-bromo-2,4-dichloro-7H-pyrrolo[2,3-d]pyrimidine (0.62 g, 2.30 mmol), compound 234 (1.27 g, 3.45 mmol), PPh₃ (1.22 g, 4.60 mmol), and DBAD (0.56 M, 1.07 g, 4.60 mmol) in dry toluene (0.40 M, 5.70 mL) after column chromatography on silica (petrol ether/THF; 0-30%) as yellowish foam (525 mg, 37%). ¹H NMR (400 MHZ, DMSO-d₆) δ8.45 (s, 1H, H6), 7.39 (t, J=6.2 Hz, 1H, —CH₂NHCO₂C(CH₃)₃), 7.34-7.23 (m, 1H, H2, H4, H5 aryl), 7.16-7.11 (m, 1H, H6 aryl), 5.28-5.17 (m, 1H, H1′), 4.94 (dd, J=7.5, 5.2 Hz, 1H, H2′), 4.73 (t, J=7.2 Hz, 1H, H3′), 4.13 (d, J=6.2 Hz, 2H, —CH₂NHCO₂C(CH₃)₃), 3.32-3.25 (m, 1H, H4′), 2.58-2.51 (m, 2H, H5′), 1.54 (s, 3H, CH₃, acetonide), 1.40 (s, 9H, —NHCO₂C(CH₃)₃), 1.22 (s, 3H, CH₃, acetonide).

### tert-butyl 2-((3αR,4R,6R,6αS)-6-(4-chloro-7H-pyrrolo[2,3-a]pyrimidin-7-yl)-2,2-dimethyltetrahydro-4H-cyclopenta[d][1,3]dioxol-4-yl) pyrrolidine-1-carboxylate (255)

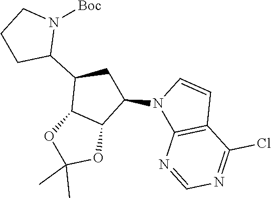 [SMILES: CC(C)(C)OC(=O)N1CCCC1[C@H]1C[C@@H](n2ccc3c(Cl)ncnc32)[C@@H]2OC(C)(C)O[C@@H]21]

Following the general procedure L for Mitsunobu-type glycosylation, compound 255 was obtained starting from 4-chloro-7H-pyrrolo[2,3-d]pyrimidine (0.10 g, 0.62 mmol), compound 235 (0.31 g, 0.93 mmol), PPh₃ (0.33 g, 1.24 mmol), and DBAD (1.10 M, 0.29 g, 1.24 mmol) in dry toluene (0.20 M, 3.10 mL) after column chromatography on silica (petrol ether/THF; 0-30%) as white foam (diastereomeric mix, 182 mg, 64%, d.r. 1.5:1). R^{f}=0.24 (petrol ether/THF; 30%). 1H NMR (400 MHZ, DMSO-d₆) δ 8.65 (s, 2H, H2), 8.03 (d, J=3.7 Hz, 1H, H6), 7.96 (bs, 1H, H6), 6.74 (d, J=3.6 Hz, 2H, H5), 5.10-4.90 (m, 3H, H1′, H2′), 4.89-4.66 (m, 1H, H3′), 4.60-4.52 (m, 1H, H3′), 4.47-4.16 (m, 1H, H2′), 4.06-3.96 (m, 1H, —NC H—), 3.96-3.89 (m, 1H, —NC H—), 3.41-3.12 (m, 4H, —NC H₂CH₂CH₂—), 2.47-2.05 (m, 6H, H4, H5′), 2.03-1.66 (m, 8H, —NC H₂CH₂CH₂—), 1.47 (s, 3H, CH₃, acetonide), 1.48-1.36 (m, 15H, —NCO₂C(CH₃)₃, CH₃, acetonide), 1.34 (s, 9H, —NCO₂C(CH₃)₃), 1.23 (s, 3H, CH₃, acetonide). APCI calc.: 462.20; found: 463.00/465.00 [M+H]⁺.

### tert-butyl (R)-2-((3αR,4R,6R,6αS)-6-(4-chloro-7H-pyrrolo[2,3-d]pyrimidin-7-yl)-2,2-dimethyltetrahydro-4H-cyclopenta[d][1,3]dioxol-4-yl) piperidine-1-carboxylate (256)

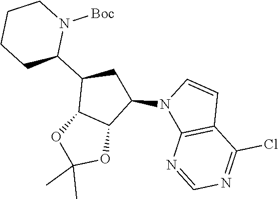 [SMILES: CC(C)(C)OC(=O)N1CCCC[C@@H]1[C@H]1C[C@@H](n2ccc3c(Cl)ncnc32)[C@@H]2OC(C)(C)O[C@H]12]

Following the general procedure L for Mitsunobu-type glycosylation, compound 256 was obtained starting from 4-chloro-7H-pyrrolo[2,3-d]pyrimidine (0.11 g, 0.71 mmol), compound 236 (0.37 g, 1.06 mmol), PPh₃ (0.38 g, 1.42 mmol), and DBAD (1.10 M, 0.33 g, 1.42 mmol) in dry toluene (0.50 M, 1.40 mL) after column chromatography on silica (petrol ether/THF; 0-30%) as white foam (165 mg, 49%). ¹H NMR (400 MHZ, DMSO-d₆) δ8.64 (s, 1H, H2), 7.93 (d, J=22.6 Hz, 1H, H6), 6.73 (d, J=3.7 Hz, 1H, H5), 5.18-4.94 (m, 2H, H1′, H2′), 4.54-4.46 (m, 1H, H3′), 4.29-4.07 (m, 1H, —CHNCO₂C(CH₃)₃), 3.92-3.71 (m, 1H, —NCO₂C(CH₃)₃CH₂—), 2.97-2.68 (m, 2H, NCO₂C(CH₃)₃CH₂—, H4′), 2.31-2.19 (m, 1H, H5′), 2.10-2.01 (m, 1H, H5′), 1.83 (d, J=11.7 Hz, 1H, —NCO₂C(CH₃)₃CH₂CH₂—), 1.57 (s, 5H, —NCO₂C(CH₃)₃CH₂CH₂CH₂CH₂—), 1.49 (s, 3H, CH₃, acetonide), 1.30 (s, 9H, —NCO₂C(CH₃)₃), 1.22 (s, 3H, CH₃, acetonide).

### tert-butyl 3-((3αR,4R,6R,6αS)-6-(5-iodo-7H-pyrrolo[2,3-a]pyrimidin-7-yl)-2,2-dimethyltetrahydro-4H-cyclopenta[d][1,3]dioxol-4-yl) piperidine-1-carboxylate (257)

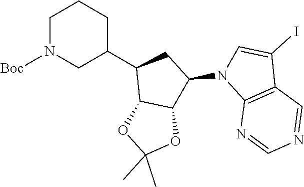 [SMILES: CC(C)(C)OC(=O)N1CCCC([C@H]2C[C@@H](n3cc(I)c4cncnc43)[C@@H]3OC(C)(C)O[C@@H]32)C1]

Following the general procedure L for Mitsunobu-type glycosylation, compound 257 was obtained starting from 5-iodo-7H-pyrrolo[2,3-d]pyrimidine (0.20 g, 0.81 mmol), compound 197 (0.42 g, 1.21 mmol), PPh₃ (0.43 g, 1.62 mmol), and DBAD (1.10 M, 0.38 g, 1.62 mmol) in dry toluene (0.50 M, 1.60 mL) after column chromatography on silica (petrol ether/THF; 0-30%) as white foam (269 mg, 59%).

### tert-butyl 3-((3αR,4R,6R,6αS)-2,2-dimethyl-6-(5-(thiazol-2-yl)-7H-pyrrolo[2,3-d]pyrimidin-7-yl) tetrahydro-4H-cyclopenta[d][1,3]dioxol-4-yl) piperidine-1-carboxylate (258)

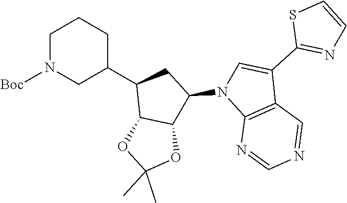 [SMILES: CC(C)(C)OC(=O)N1CCCC([C@H]2C[C@@H](n3cc(-c4nccs4)c4cncnc43)[C@@H]3OC(C)(C)O[C@@H]32)C1]

Compound 257 (0.26 g, 0.45 mmol) was added to a heat-dried vial under nitrogen. Then, Pd(Ph₃)₂Cl₂ (0.03 g, 0.05 mmol) was added under nitrogen. Afterwards, dry toluene (4.50 mL) was added and the resulted mixture was degassed. At last, 2-(tributylstannyl) thiazole (0.29 mL, 0.90 mmol) was added and the mixture was heated to 100° C. and stirred for 17 h. The reaction mixture was allowed to cool down to room temperature and concentrated to complete dryness. The crude product was purified over silica gel chromatography (0-100%) to afford the title product (150 mg, 64%).

### tert-butyl 3-((3αR,4R,6R,6αS)-6-(4-(difluoromethyl)-7H-pyrrolo[2,3-d]pyrimidin-7-yl)-2,2-dimethyltetrahydro-4H-cyclopenta[d][1,3]dioxol-4-yl) piperidine-1-carboxylate (259)

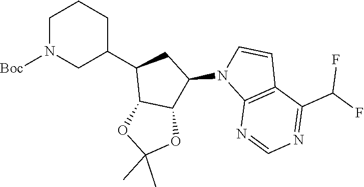 [SMILES: CC(C)(C)OC(=O)N1CCCC([C@H]2C[C@@H](n3ccc4c(C(F)F)ncnc43)[C@@H]3OC(C)(C)O[C@@H]32)C1]

Following the general procedure L for Mitsunobu-type glycosylation, compound 259 was obtained starting from 4-(difluoromethyl)-7H-pyrrolo[2,3-d]pyrimidine (0.13 g, 0.73 mmol), compound 197 (0.38 g, 1.10 mmol), PPh₃ (0.39 g, 1.46 mmol), and DBAD (1.10 M, 0.34 g, 1.46 mmol) in dry toluene (0.20 M, 3.70 mL) after column chromatography on silica (cyclohexane/EtOAc; 0-30%) as white foam (199 mg, 55%).

### 7-((3αS,4R,6R,6αR)-6-(3-((tert-butyldimethylsilyl)oxy)phenyl)-2,2-dimethyltetrahydro-4H-cyclopenta[d][1 3]dioxol-4-yl)-4-chloro-7H-pyrrolo [2,3-d]pyrimidine (260)

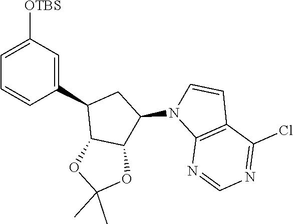 [SMILES: CC1(C)O[C@@H]2[C@H](O1)[C@@H](c1cccc(O[Si](C)(C)C(C)(C)C)c1)C[C@H]2n1ccc2c(Cl)ncnc21]

In the first step, PPh₃ (521 mg, 1.99 mol, 2 eq.) and DBAD (454 mg, 1.97 mol, 2 eq.) in toluene (10 mL) were stirred for 30 min at 0° C. under N₂ atmosphere. A solution of (3αS,4S,6R,6αR)-6-(3-((tert-butyldimethylsilyl)oxy)phenyl)-2,2-dimethyltetrahydro-4H-cyclopenta[d][1,3]dioxol-4-ol (360 mg, 0.99 mol, 1 eq.) in toluene (5 mL) was added dropwise and stirred for 30 min at 0° C. Then, 4-chloro-7Hpyrrolo[2,3-d]pyrimidine (153 mg, 1.00 mol, 1 eq.) was added to the reaction mixture and heated up to 80° C. and stirred for 15.5 h. After the reaction mixture was allowed to cool down to RT, the solvent was evaporated. Then, water was added and the residue was extracted three times with EtOAc, the combined organic phases were once washed with brine and then dried over Na₂SO₄. After filtration and concentration, the residue was purified by flash chromatography eluting with PE/EtOAc (0.2%->4%) to give 7-((3αS,4R,6R,6αR)-6-(3 ((tertbutyldimethylsilyl)oxy)phenyl)-2,2-dimethyltetrahydro-4Hcyclopenta[d][1,3]dioxol-4-yl)-4-chloro-7Hpyrrolo[2,3-d]pyrimidine (221 mg) as a transparent oil. R^{f}=0.25 (Petrolether/EtOAc 9:1). APCI: calcd. for C26H34ClN303Si [M+H]⁺: 500.2 found 499.9/441.

### 7-((3αS,4R,6R,6αR)-6-(4-((tert-butyldimethylsilyl)oxy)phenyl)-2,2-dimethyltetrahydro-4H cyclopenta[d][1.3]dioxol-4-yl)-4-chloro-7H-pyrrolo [2,3-d]pyrimidine (261)

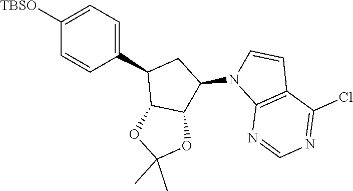 [SMILES: CC1(C)O[C@@H]2[C@H](O1)[C@@H](c1ccc(O[Si](C)(C)C(C)(C)C)cc1)C[C@H]2n1ccc2c(Cl)ncnc21]

In the first step, PPh₃ (721 mg, 2.74 mmol, 2 eq.) and DBAD (632 mg, 2.75 mmol, 2 eq.) were dissolved in toluene (7 mL) at 0° C. under nitrogen and stirred for 30 min. A solution of (3αS,4S,6R,6αR)-6-(4-((tert-butyldimethylsilyl)oxy)phenyl)-2,2-dimethyltetrahydro-4H-cyclopenta[d][1,3]dioxol-4-ol (504 mg, 1.38 mmol, 1 eq.) in toluene (7 mL) was added at 0° C. After 30 min stirring at this temperature, 4-chloro-7H-pyrrolo[2,3-d]pyrimidine (216 mg, 1.41 mmol, 1 eq.) was added to the yellow solution. The reaction mixture was stirred for 17.6 h at 110° C. After evaporation of the solvent, water was added and the mixture was extracted three times with EtOAc. The combined organic layers were washed with brine and dried over Na₂SO₄. After filtration and concentration, the residue was purified twice by flash chromatography eluting with Petrolether/EtOAc (1->5%) to give 7-((3αS,4R,6R,6αR)-6-(4-((tert-butyldimethylsilyl)oxy)phenyl)-2,2-dimethyltetrahydro-4H-cyclopenta[d][1,3]dioxol-4-yl)-4-chloro-7H-pyrrolo[2,3-d]pyrimidine (300 mg), as a viscose and transparent oil. R^{f}=0.10 (Petrolether/EtOAc 9:1). APCI: calcd. for C26H34ClN303Si [M+H]⁺: 500.2 found 500.0

### 4-chloro-7-((3αS,4R,6R,6αR)-6-(3-chlorophenyl)-2,2-dimethyltetrahydro-4H-cyclopenta[d][1,3]dioxol-4-yl)-7H-pyrrolo[2,3-a]pyrimidine (262)

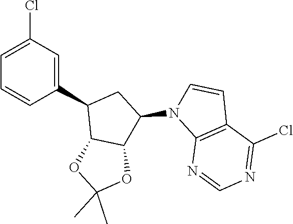 [SMILES: CC1(C)O[C@@H]2[C@H](O1)[C@@H](c1cccc(Cl)c1)C[C@H]2n1ccc2c(Cl)ncnc21]

To a stirred solution of PPh₃ (237 mg, 0.90 mmol, 2 eq.), 4-chloro-7H-pyrrolo[2,3-d]pyrimidine (69 mg, 0.45 mmol, 1 eq.) and (3αS,4S,6R,6αR)-6-(3-chlorophenyl)-2,2-dimethyltetrahydro-4H-cyclopenta[d][1,3]dioxol-4-ol (181 mg, 0.67 mmol, 1.5 eq.) in toluene (4 mL) was added DBAD (207 mg, 0.90 mmol, 2 eq.) portionwise at 0° C. under N₂. The reaction mixture was stirred at 0° C. for 10 min, then it was heated up to 60° C. and stirred for 22 h at this temperature. Then, the mixture was concentrated under reduced pressure and was purified by flash chromatography eluting with Petrolether/EtOAc (1->10%) to give 4-chloro-7-((3αS,4R,6R,6αR)-6-(3-chlorophenyl)-2,2-dimethyltetrahydro-4H-cyclopenta[d][1,3]dioxol-4-yl)-7H-pyrrolo[2,3-d]pyrimidine (125 mg), as a transparent oil. APCI: calcd. for C20H19Cl₂N₃₀₂ [M+H]⁺: 404.1 found 403.6.

### 4-chloro-7-((3αS,4R,6R,6αR)-2,2-dimethyl-6-(3-(2-methyl-1,3-dioxolan-2-yl)phenyl) tetrahydro-4H-cyclopenta[d][1,3]dioxol-4-yl)-7H-pyrrolo[2,3-d]pyrimidine (263)

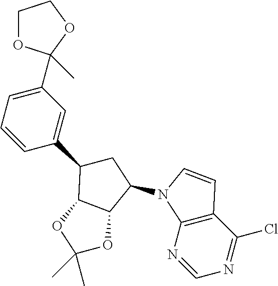 [SMILES: CC1(C)O[C@@H]2[C@H](O1)[C@@H](c1cccc(C3(C)OCCO3)c1)C[C@H]2n1ccc2c(Cl)ncnc21]

To a stirred solution of PPh₃ (213 mg, 0.81 mmol, 2 eq.), 4-chloro-7H-pyrrolo[2,3-d]pyrimidine (61 mg, 0.40 mmol, 1 eq.) in toluene (0.6 mL) was added solution of (3αS,4S,6R,6αR)-2,2-dimethyl-6-(3-(2-methyl-1,3-dioxolan-2-yl)phenyl) tetrahydro-4H-cyclopenta[d][1,3]dioxol-4-ol (188 mg, 0.59 mmol, 1.5 eq.) in toluene (1.4 ml) under N₂. To this, solution of DBAD (180 mg, 0.78 mmol, 2 eq.) in toluene (1.6 ml) was added dropwise at 0° C. under N₂. The reaction mixture was stirred at 0° C. for 20 min, then it was heated up to 60° C. and stirred for 4 h at this temperature. Then, the mixture was concentrated under reduced pressure and was purified by flash chromatography eluting with Cyclohexane/EtOAc (1-+20%) to give 4-chloro-7-((3αS,4R,6R,6αR)-2,2-dimethyl-6-(3-(2-methyl-1,3-dioxolan-2-yl)phenyl) tetrahydro-4H-cyclopenta[d][1,3]dioxol-4-yl)-7H-pyrrolo[2,3-d]pyrimidine (200 mg), as a transparent foam. APCI: calcd. for C₂₄H₂₆ClN₃O₄ [M+H]⁺: 456.2 found 455.8.

### 7-((3αS,4R,6R,6αR)-6-(3,5-dichlorophenyl)-2,2-dimethyltetrahydro-4H-cyclopenta[d][1,3]dioxol-4-yl)-7H-pyrrolo[2,3-a]pyrimidin-4-amine (264)

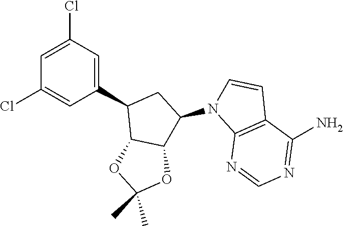

Following the general procedure M for aromatic substitution, compound 264 was obtained starting from compound 244 (0.19 g, 0.43 mmol) in a mixture of 1,4-dioxane and ammonia (0.05 M, 8.60 mL, 1:2) after column chromatography on silica (cyclohexane/EtOAc; 0-100%) as white foam (160 mg, 98%). ¹H NMR (400 MHZ, DMSO-d₆) δ8.07 (s, 1H, H2), 7.52-7.47 (m, 4H, H6, o,p-Ar), 7.00 (s, 2H, —CN H₂), 6.60 (d, J=3.5 Hz, 1H, H5), 5.14 (td, J=9.5, 5.1 Hz, 1H, H1′), 4.89 (dd, J=7.6, 5.2 Hz, 1H, H2′), 4.76 (t, J=7.2 Hz, 1H, H3′), 3.32-3.27 (m, 1H, H4′), 2.53-2.44 (m, 2H, H5′under DMSO peak), 1.52 (s, 3H, CH₃, acetonide), 1.22 (s, 3H, CH₃, acetonide).

### 5-bromo-7-((3αS,4R,6R,6αR)-6-(3,5-dichlorophenyl)-2,2-dimethyltetrahydro-4H-cyclopenta[d][1,3]dioxol-4-yl)-7H-pyrrolo[2,3-a]pyrimidin-4-amine (265)

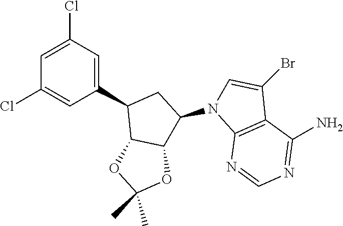

Following the general procedure M for aromatic substitution, compound 265 was obtained starting from compound 245 (0.24 g, 0.45 mmol) in a mixture of 1,4-dioxane and ammonia (0.05 M, 9.00 mL, 1:2) after column chromatography on silica (cyclohexane/EtOAc; 0-100%) as white foam (88 mg, 39%). ¹H NMR (400 MHZ, DMSO-d₆) δ8.12 (s, 1H, H2 adenine), 7.84 (s, 1H, H6 adenine), 7.52-7.49 (m, 3H,,o,p-Ar), 5.19 (td, J=9.3, 5.0 Hz, 1H, H1′), 4.85 (dd, J=7.5, 5.0 Hz, 1H, H2′), 4.75-4.69 (m, 1H, H3′), 3.32-3.25 (m, 1H, H4′ under water peak), 2.52-2.44 (m, 2H, H5′ under DMSO peak), 1.52 (s, 3H, CH₃, acetonide), 1.21 (s, 3H, CH₃, acetonide).

### 7-((3αS,4R,6R,6αR)-2,2-dimethyl-6-phenyltetrahydro-4//-cyclopenta[d][1,3]dioxol-4-yl)-7H-pyrrolo[2,3-d]pyrimidin-4-amine (266)

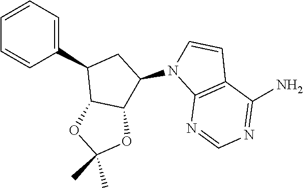

Following the general procedure M for aromatic substitution, compound 266 was obtained starting from compound 246 (0.18 g, 0.45 mmol) in a mixture of 1,4-dioxane and ammonia (0.05 M, 9.60 mL, 1:2) after column chromatography on silica (cyclohexane/EtOAc; 0-100%) as white foam (72 mg, 89%). ¹H NMR (400 MHZ, DMSO-d₆) δ8.07 (s, 1H, H2), 7.44 (d, J=3.6 Hz, 1H, H6), 7.41-7.32 (m, 4H, o,m-Ar), 7.29-7.22 (m, 1H, p-Ar), 6.98 (s, 2H, —CN H₂), 6.59 (d, J=3.6 Hz, 1H, H5), 5.12 (ddd, J=10.8, 8.1, 5.4 Hz, 1H, H1′), 4.96 (dd, J=7.6, 5.4 Hz, 1H, H2′), 4.75 (t, J=7.2 Hz, 1H, H3′), 3.29-3.22 (m, 1H, H4′), 2.54-2.45 (m, 2H, H5′ under DMSO peak), 1.52 (s, 3H, CH₃, acetonide), 1.22 (s, 3H, CH₃, acetonide).

### 5-bromo-7-((3αS,4R,6R,6αR)-2,2-dimethyl-6-phenyltetrahydro-4H-cyclopenta[d][1,3]dioxol-4-yl)-7H-pyrrolo[2.3-d]pyrimidin-4-amine (267)

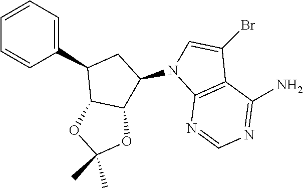

Following the general procedure M for aromatic substitution, compound 267 was obtained starting from compound 247 (0.20 g, 0.44 mmol) in a mixture of 1,4-dioxane and ammonia (0.20 M, 2.20 mL, 1:2) after column chromatography on silica (cyclohexane/EtOAc; 0-100%) as white foam (72 mg, 89%). R^{f}=0.69 (CH₂Cl₂/MeOH; 10%). ¹H NMR (400 MHZ, DMSO-d₆) δ8.13 (s, 1H, H2), 7.81 (s, 1H, H6), 7.43-7.31 (m, 4H, o, m-Ar), 7.31-7.22 (m, 1H, p-Ar), 5.17 (td, J=9.8, 9.3, 5.3 Hz, 1H, H1′), 4.92 (dd, J=7.5, 5.3 Hz, 1H, H2′), 4.72 (t, J=7.2 Hz, 1H, H3′), 3.29-3.16 (m, 1H, H4′), 2.53-2.47 (m, 2H, H5′ under DMSO peak), 1.52 (s, 3H, CH₃, acetonide), 1.22 (s, 3H, CH₃, acetonide).

### 7-((3αS,4R,6R,6αR)-2,2-dimethyl-6-(naphthalen-2-yl) tetrahydro-4H/-cyclopenta[d][1,3]dioxol-4-yl)-7H pyrrolo[2,3d]pyrimidin-4-amine (268)

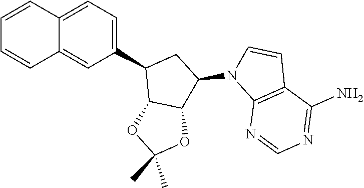

Following the general procedure M for aromatic substitution, compound 268 was obtained starting from compound 248 (0.20 g, 0.47 mmol) in a mixture of 1,4-dioxane and ammonia (0.20 M, 2.40 mL, 1:2) after column chromatography on silica (cyclohexane/EtOAc; 0-100%) as white foam (72 mg, 89%). R_{f}=0.73 (CH₂Cl₂/MeOH; 10%). ¹H NMR (400 MHZ, DMSO-d₆) δ 8.09 (s, 1H, H2 adenine), 7.96-7.83 (m, 4H, H1, H4, H5, H8 naphthyl), 7.70 (dd, J=3.5, 2.4 Hz, 1H, H6 adenine), 7.61 (dd, J=8.5, 1.8 Hz, 1H, H3 naphthyl), 7.55-7.45 (m, 2H, H6, H7 naphthyl), 6.62-6.60 (m, 1H, H5 adenine), 5.19 (dt,)=12.0, 6.2 Hz, 1H, H1′), 5.01 (dd, J=7.5, 5.4 Hz, 1H, H2′), 4.86 (t, J=7.2 Hz, 1H, H3′), 3.52-3.40 (m, 1H, H4′), 2.73-2.55 (m, 2H, H5′), 1.56 (s, 3H, CH₃, acetonide), 1.25 (s, 3H, CH₃, acetonide).

### 2-chloro-7-((3αS,4R,6R,6αR)-2,2-dimethyl-6-(naphthalen-2-yl) tetrahydro-4H-cyclopenta[d][1,3]dioxol-4-yl)-7H-pyrrolo[2,3-d]pyrimidin-4-amine (269)

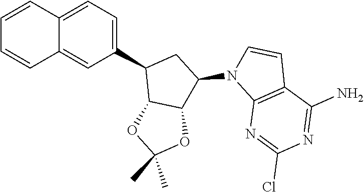

Following the general procedure M for aromatic substitution, compound 269 was obtained starting from compound 249 (0.26 g, 0.35 mmol) in a mixture of 1,4-dioxane and ammonia (0.10 M, 3.50 mL, 1:2) after column chromatography on silica (cyclohexane/EtOAc; 0-100%) as white foam (242 mg, 0.56 mmol, impurity in cyclohexane). R^{f}=0.60 (cyclohexane/EtOAc; 70%). ¹H NMR (400 MHZ, DMSO-d₆) δ7.96-7.84 (m, 4H, H1, H4, H5, H8 naphthyl), 7.62 (dd, J=8.4, 1.6 Hz, 1H, H6 naphthyl), 7.57-7.44 (m, 5H, —CN H₂, H3, H7 naphthyl, H6 adenine), 6.64 (d, J=3.6 Hz, 1H, H5 adenine), 5.16-5.06 (m, 1H, H1′), 4.94 (dd, J=7.4, 5.2 Hz, 1H, H2′), 4.88-4.83 (m, 1H, H3′), 3.51-3.44 (m, 1H, H4′), 2.68-2.65 (m, 1H, H5′), 2.62-2.58 (m, 1H, H5′), 1.57 (s, 3H, CH₃, acetonide), 1.25 (s, 3H, CH₃, acetonide). APCI calc.: 394.12; found: 434.90/436.90 [M+H]⁺.

### 7-((3αS,4R,6R,6αR)-2,2-dimethyl-6-(pyridin-4-yl) tetrahydro-4H-cyclopenta[d][1,3]dioxol-4-yl)-7H-nvrrolnl2 3-dIpyrimidin-4-amine (270)

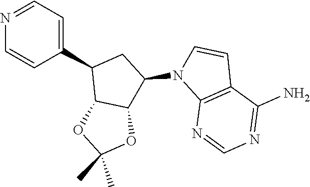

Following the general procedure M for aromatic substitution, compound 270 was obtained starting from compound 250 (0.35 g, 0.93 mmol) in a mixture of 1,4-dioxane and ammonia (0.10 M, 9.40 mL, 1:2) after column chromatography on silica (CH₂Cl₂; 0-10%) as white foam (66.20 mg, 20%). R^{f}=0.59 (CH₂Cl₂/MeOH; 10%). ¹H NMR (400 MHZ, DMSO-d₆) δ8.55-8.51 (m, 2H, H2, H6 pyridine), 8.07 (s, 1H, H2 adenine), 7.44 (d, J=3.6 Hz, 1H, H6 adenine), 7.42-7.39 (m, 2H, H3, H5 pyridine), 7.00 (s, 2H, —CN H₂), 6.60 (d, J=3.5 Hz, 1H, H5 adenine), 5.14 (ddd, J=11.0, 7.8, 5.2 Hz, 1H, H1′), 4.95 (dd, J=7.5, 5.2 Hz, 1H, H2′), 4.79 (t, J=7.2 Hz, 1H, H3′), 3.31-3.24 (m, 1H, H4′), 2.52 (s, 2H, H5′ under DMSO peak), 1.53 (s, 3H, CH₃, acetonide), 1.23 (s, 3H, CH₃, acetonide).

### 7-((3αS,4R,65,6αR)-2,2-dimethyl-6-(thiophen-2-yl) tetrahydro-4//-cyclopenta[d][1,3]dioxol-4-yl)-7H-pyrrolo[2.3-d]pyrimidin-4-amine (271)

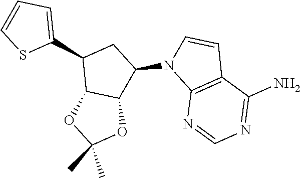

Following the general procedure M for aromatic substitution, compound 271 was obtained starting from compound 251 (0.20 g, 0.52 mmol) in a mixture of 1,4-dioxane and ammonia (0.10 M, 5.20 mL, 1:2) after column chromatography on silica (cyclohexane/EtOAc; 0-100%) as white foam (27 mg, 15%). R^{f}=0.67 (CH₂Cl₂/MeOH; 10%). ¹H NMR (400 MHZ, DMSO-d₆) δ 8.06 (s, 1H, H2), 7.42-7.38 (m, 2H, H6, H5 thiophene), 7.03-6.96 (m, 4H, —CN H₂, H3, H4 thiophene), 6.58 (d, J=3.5 Hz, 1H, H5), 5.15-5.04 (m, 1H, H1′), 4.96 (dd, J=7.5, 5.2 Hz, 1H, H2′), 4.73 (t, J=7.2 Hz, 1H, H3′), 3.49 (dt, J=11.3, 7.3 Hz, 1H, H4′), 2.60-2.52 (m, 2H, H5′), 1.52 (s, 3H, CH₃, acetonide), 1.23 (s, 3H, CH₃, acetonide).

### 7-((3αS,4R,6R,6αR)-2,2-dimethyl-6-(1H-pyrazol-5-yl) tetrahydro-4//-cyclopenta[d][1,3]dioxol-4-yl)-7H-pyrrolo[2,3-a]pyrimidin-4-amine (272)

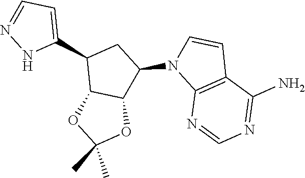

Following the general procedure M for aromatic substitution, compound 272 was obtained starting from compound 252 (0.26 g, 0.50 mmol) in a mixture of 1,4-dioxane and ammonia (0.10 M, 5.00 mL, 1:2) after column chromatography on silica (CH₂Cl₂, MeOH; 0-20%) as white foam (27 mg, 0.08 mmol). R^{f}=0.43 (CH₂Cl₂, MeOH; 10%). APCI calc.: 340.16; found: 340.90 [M+H]⁺.

### 2-chloro-7-((3αS,4R,6R,6αR)-2,2-dimethyl-6-(quinolin-6-yl) tetrahydro-4H-cyclopenta[d][1,3]dioxol-4-yl)-7H-pyrrolo[2,3-d]pyrimidin-4-amine (273)

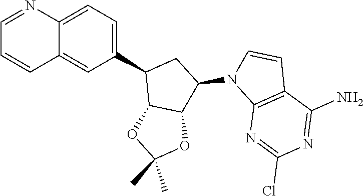

Following the general procedure M for aromatic substitution, compound 273 was obtained starting from compound 253 (0.07 g, 0.15 mmol) in a mixture of 1,4-dioxane and ammonia (0.10 M, 1.50 mL, 1:2) after column chromatography on silica (cyclohexane/EtOAc; 0-100%) as white foam (53 mg, 80%). ¹H NMR (400 MHZ, DMSO-d₆) δ8.87 (dd, J=4.1, 1.6 Hz, 1H, H2 quinoline), 8.35 (d, J=8.3 Hz, 1H, H4 quinoline), 8.03 (d, J=8.8 Hz, 1H, H8 quinoline), 7.94 (d, J=10.5 Hz, 1H, H5 quinoline), 7.86 (dd, J=8.7, 1.8 Hz, 1H, H7 quinoline), 7.58-7.50 (m, 2H, H3 quinoline, H6 adenine), 6.64 (d, J=3.6 Hz, 1H, H5 adenine), 5.13 (dt, J=11.8, 6.6 Hz, 1H, H1′), 4.94 (dd, J=7.3, 5.2 Hz, 1H, H2′), 4.87 (t, J=7.1 Hz, 1H, H3′), 3.51 (dt, J=13.1, 6.9 Hz, 1H, H4′), 2.65-2.53 (m, 2H, H5′), 1.57 (s, 3H, CH₃, acetonide), 1.25 (s, 3H, CH₃, acetonide). APCI calc.: 435.15; found: 435.60/437.60 [M+H]⁺.

### tert-butyl (3-((3αR,4R,6R,6αS)-6-(4-amino-5-bromo-2-chloro-7H-pyrrolo[2,3-d]pyrimidin-7-yl)-2,2-dimethyltetrahydro-4H-cyclopenta[d][1,3]dioxol-4-yl)benzyl)carbamate (274)

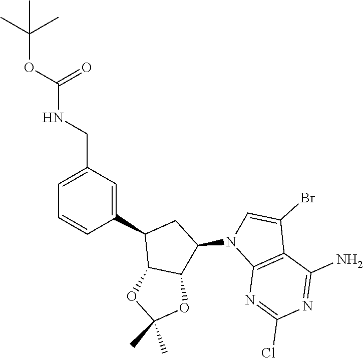

Following the general procedure M for aromatic substitution, compound 274 was obtained starting from compound 254 (0.52 g, 0.84 mmol) in a mixture of 1,4-dioxane and ammonia (0.10 M, 8.40 mL, 1:2) after column chromatography on silica (n-heptane/EtOAc; 0-100%) as white foam (230 mg, 46%). ¹H NMR (400 MHZ, DMSO-d₆) δ7.84 (s, 1H, H6 adenine), 7.39 (t, J=6.2 Hz, 1H, —CH₂NHCO₂C(CH₃)₃), 7.32-7.22 (m, 3H, H2, H4, H5 aryl), 7.15-7.10 (m, 1H, H6 aryl), 5.11-5.03 (m, 1H, H1′), 4.87 (dd, J=7.5, 5.3 Hz, 1H, H2′), 4.70 (t, J=7.2 Hz, 1H, H3′), 4.13 (d, J=6.2 Hz, 2H, —CH₂NHCO₂C(CH₃)₃), 3.24 (dt, J=13.6, 6.6 Hz, 1H, H4′), 2.48-2.38 (m, 2H, H5′), 1.52 (s, 3H, CH₃, acetonide), 1.39 (s, 9H, —NHCO₂C(CH₃)₃), 1.22 (s, 3H, CH₃, acetonide).

### tert-butyl 2-((3αR,4R,6R,6αS)-6-(4-amino-7H-pyrrolo[2,3-d]pyrimidin-7-yl)-2,2-dimethyltetrahydro-4H-cyclopenta[d][1,3]dioxol-4-yl) pyrrolidine-1-carboxylate (275)

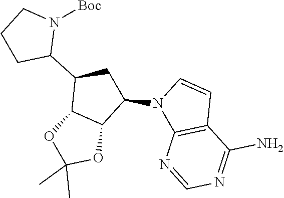 [SMILES: CC(C)(C)OC(=O)N1CCCC1[C@H]1C[C@@H](n2ccc3c(N)ncnc32)[C@@H]2OC(C)(C)O[C@@H]21]

Following the general procedure M for aromatic substitution, compound 275 was obtained starting from compound 255 (0.16 g, 0.34 mmol) in a mixture of 1,4-dioxane and ammonia (0.10 M, 3.40 mL, 1:2) after column chromatography on silica (CH₂Cl₂/MeOH; 0-10%) afforded the desired product as white foam (101 mg, 67%, diastereomeric mixture; d.r. 1.5:1). R^{f}=0.68 (CH₂Cl₂/MeOH; 10%). ¹H NMR (400 MHZ, DMSO) δ8.04 (s, 2H, H2), 7.34 (d, J=3.5 Hz, 1H, H6), 7.31-7.25 (m, 1H, H6), 6.98 (bs, 2H, 2H, —CN H₂), 6.57-6.54 (m, 2H, H5), 4.96-4.75 (m, 4H, H1′, H2′), 4.55-4.47 (m, 2H, H3′), 4.01-3.93 (m, 1H, —CH₂CH₂CHCH—), 3.93-3.85 (m, 1H, —CH₂CH₂CHCH—), 3.50-3.37 (m, 1H, —NC H₂CH₂CH₂—), 3.26-3.11 (m, 1H, —NC H₂CH₂CH₂—), 2.40-2.26 (m, 2H, H4′, H5′), 2.25-2.14 (m, 2H, H4′, H5′), 2.12-2.00 (m, 2H, H5′), 1.95-1.71 (m, 8H, —NC H₂CH₂CH₂—), 1.44 (s, 6H, CH₃, acetonide), 1.42 (s, 9H, —NCO₂C(CH₃)₃), 1.33 (s, 9H, —NCO₂C(CH₃)₃), 1.21 (s, 6H, CH₃, acetonide). APCI calcd.: 443.25; found: 444.30 [M+H]⁺.

### tert-butyl (R)-2-((3αR,4R,6R,6αS)-6-(4-amino-7//pyrrolo[2,3-d]pyrimidin-7-yl)-2,2-dimethyltetrahydro-4H-cyclopenta[d][1,3]dioxol-4-yl) piperidine-1-carboxylate (276)

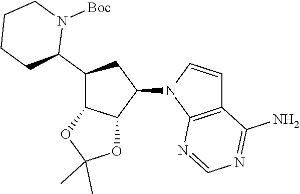 [SMILES: CC(C)(C)OC(=O)N1CCCC[C@@H]1[C@H]1C[C@@H](n2ccc3c(N)ncnc32)[C@@H]2OC(C)(C)O[C@H]12]

Following the general procedure M for aromatic substitution, compound 276 was obtained starting from compound 256 (0.15 g, 0.31 mmol) in a mixture of 1,4-dioxane and ammonia (0.10 M, 3.10 mL, 1:2) after column chromatography on silica (CH₂Cl₂/MeOH; 0-10%) afforded the desired product as white foam (52 mg, 37%, 43 mg starting material recovered). R^{f}=0.38 (CH₂Cl₂/MeOH; 10%). ¹H NMR (400 MHZ, DMSO-d₆) δ8.03 (s, 1H, H2), 7.25 (d, J=20.2 Hz, 1H, H6), 6.97 (s, 2H, —NH₂), 6.55 (d, J=3.4 Hz, 1H, H5), 5.05-4.77 (m, 2H, H1′, H2′), 4.46 (t, J=6.7 Hz, 1H, H3′), 4.25-4.04 (m, 1H, —CH₂CH₂CH₂CH—), 3.95-3.71 (m, 1H, —NC H₂CH₂CH₂CH₂—), 2.93-2.58 (m, 1H, —NC H₂CH₂CH₂CH₂—, H4′), 2.38-2.22 (m, 1H, H5′), 2.03-1.91 (m, 1H, H5′), 1.83 (d, J=12.0 Hz, 1H, —NC H₂CH₂CH₂CH₂—), 1.63-1.41 (m, 5H, —NC H₂CH₂CH₂CH₂—), 1.48 (s, 3H, CH₃, acetonide), 1.30 (s, 9H, —NCO₂C(CH₃)₃), 1.22 (s, 3H, CH₃, acetonide).

### 3-((3αR,4R,6R,6αS)-6-(4-amino-7H-pyrrolo[2,3-d]pyrimidin-7-yl)-2,2-dimethyltetrahydro-4H cyclopenta[d][1,3]dioxol-4-yl)phenol (277)

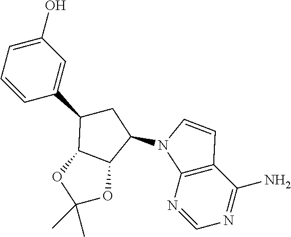 [SMILES: CC1(C)O[C@@H]2[C@H](O1)[C@@H](c1cccc(O)c1)C[C@H]2n1ccc2c(N)ncnc21]

The product from previous step (210) was dissolved in dioxane (0.5 mL) in a pressure flask. Aqueous ammonia (25%, 0.5 mL) was added and the solution was stirred for 16 h at 100° C. After evaporation under reduced pressure, the residue was purified by flash chromatography eluting DCM/MeOH and besides the corresponding product, TBS deprotected, but not substituted intermediate was recovered and the reaction was relaunched with aq. ammonia (50%, 1 mL) and dioxane (1 mL) for 2 days at 100° C. Then, the residue was purified by flash chromatography eluting with DCM/MeOH (0.3%->7%) to afford product (yield: 65 mg, 0.18 mmol, 13% over 2 steps), as a yellow oil. R^{f}=0.53 (DCM/MeOH 9:1). APCI: calcd. for C26H36N₄₀₃Si [M+H]⁺: 367.2 found 366.9 ¹H NMR (400 MHZ, DMSO-d₆) δ 9.34 (s, 1H, phenyl OH), 8.07 (s, 1H, H2), 7.42 (d, J=3.6 Hz, 1H, H8), 7.15-7.10 (m, 1H, m-H), 6.99 (bs, 2H, NH₂), 6.80-6.76 (m, 2H, 2 x o-H,), 6.64 (ddd, J=8.1 Hz, 2.5 Hz, 1.1 Hz, 1H, p-H), 6.59 (d, J=3.5, 1H, H7), 5.12-5.05 (m, 1H, H1′), 4.95 (dd, J=7.6 Hz, 5.4 Hz, 1H, H2′), 4.70 (t, J=7.2 Hz, 1H, H3′), 3.17 (dt, J=12.4, 6.9 Hz, 1H, H4′), 2.47-2.42 (m, 2H, H5′), 1.52 (s, 3H, CH₃, acetonide), 1.22 (s, 3H, CH₃, acetonide). 1.75 eq. of DMF (24% of the overall mass) was observed by NMR. The yield was calculated on the 76% of the overall mass, corresponding to the pure target compound.

### 4-((3αR,4R,6R,6αS)-6-(4-amino-7H-pyrrolo[2,3-d]pyrimidin-7-yl)-2,2-dimethyltetrahydro-4H cyclopenta[d][1 3]dioxol-4-yl)phenol (278)

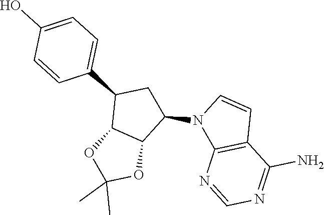 [SMILES: CC1(C)O[C@@H]2[C@H](O1)[C@@H](c1ccc(O)cc1)C[C@H]2n1ccc2c(N)ncnc21]

To a solution or the product from previous step (151 mg) in dioxane (0.54 mL) in a pressure flask was added aqueous ammonia (25%, 0.54 mL). The reaction mixture was stirred for 16.3 h at 100° C. After evaporation under reduced pressure, the residue was purified by flash chromatography eluting with DCM/MeOH (0.3->10%) to give product (yield: 30.8 mg, 0.08 mmol, 11% over 2 steps), a white to yellowish solid. R^{f}=0.34 (DCM/MeOH 9:1). APCI: calcd. for C26H36N₄₀₃Si [M+H]⁺: 367.2 found 366.8. ¹H NMR (400 MHZ, DMSO-d₆) δ 9.26 (s, 1H, phenyl OH), 8.06 (s, 1H, H2), 7.42 (d, J=3.6 Hz, 1H, H8), 7.19-7.13 (m, 2H, 2 x o-H), 6.99 (br s, 2H, NH₂), 6.76-6.70 (m, 2H, m-H), 6.59 (d, J=3.5 Hz, 1H, H7), 5.12-5.03 (m, 1H, H1’), 4.93 (dd, J=7.5 Hz, 5.5 Hz, 1H, H2′), 4.66 (t, J=7.2 Hz, 1H, H3′), 3.19-3.10 (m, 1H, H4′), 2.47-2.38 (m, 2H, H5′), 1.51 (s, 3H, CH₃, acetonide), 1.21 (s, 3H, CH₃, acetonide).

### 7-((3αS,4R,6R,6αR)-6-(4-((tert-butyldimethylsilyl)oxy)phenyl)-2,2-dimethyltetrahydro-4H-cyclopenta[d][1,3]dioxol-4-yl)-N-methyl-7H- pyrrolo[2,3-d]pyrimidin-4-amine (279)

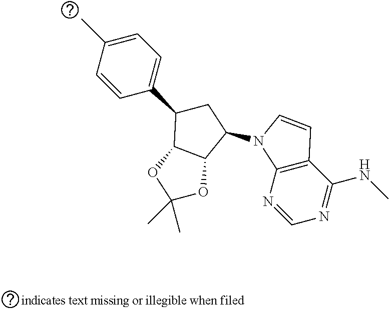

To the solution of 7-((3αS,4R,6R,6αR)-6-(4-((tert-butyldimethylsilyl)oxy)phenyl)-2,2-dimethyltetrahydro-4H-cyclopenta[d][1,3]dioxol-4-yl)-4-chloro-7H-pyrrolo[2,3-d]pyrimidine (145 mg, 0.29 mmol, 1 eq.) in n-BuOH (0.7 mL) was added solution of methylamine in EtOH (33%, 0.7 mL) in a microwave vial. The reaction mixture was stirred under u-waves irradiation at 120° C. (100 W) for 15 min. After full conversion of starting material, the solvent was evaporated and the brown residue was purified by flash chromatography eluting with Petrolether/EtOAc (2->70%) to afford 7-((3αS,4R,6R,6αR)-6-(4-((tert-butyldimethylsilyl)oxy)phenyl)-2,2-dimethyltetrahydro-4H-cyclopenta[d][1,3]dioxol-4-yl)-N-methyl-7H-pyrrolo[2,3-d]pyrimidin-4-amine (53 mg) as a brown oil. R^{f}=0.18 (Petrolether/EtOAc 1:1). APCI: calcd. for C₂₇H₃₈N₄O₃Si [M+H]⁺: 495.3 found 494.8.

### 7-((3αS,4R,6R,6αR)-6-(3-chlorophenyl)-2,2-dimethyltetrahydro-4H-cyclopenta[d][1,3]dioxol-4-yl)-7H-pyrrolo[2,3-a]pyrimidin-4-amine (280)

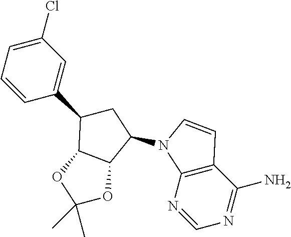 [SMILES: CC1(C)O[C@@H]2[C@H](O1)[C@@H](c1cccc(Cl)c1)C[C@H]2n1ccc2c(N)ncnc21]

To a solution of the product from previous step (113 mg) in dioxane (1.3 mL) in a pressure flask was added aqueous ammonia (50%, 1.3 mL). The reaction mixture was stirred for 67 h at 100° C. After concentration, the residue was purified by flash chromatography eluting with DCM/MeOH (0.5-+5%) to give 7-((3αS,4R,6R,6αR)-6-(3-chlorophenyl)-2,2-dimethyltetrahydro-4H-cyclopenta[d][1,3]dioxol-4-yl)-7H-pyrrolo[2,3-d]pyrimidin-4-amine (yield: 28 mg, 0.072 mmol, 11% over 2 steps), a brown oil. R^{f}=0.16 (DCM/MeOH 95:5). APCI: calcd. for C₂₀H₂₁ClN₄O₂ [M+H]*: 385.1 found 384.7 ¹H NMR (400 MHZ, DMSO-d₆) δ 8.06 (s, 1H, H2), 7.49-7.47 (m, 1H, m-H), 7.45 (d, J=3.6 Hz, H8), 7.40-7.30 (m, 3H, 2 x o-H, p-H), 6.99 (bs, 2H, NH₂), 6.59 (d, J=3.5 Hz, 1H, H7), 5.12 (td, J=9.3, 5.2 Hz, 1H, H1′), 4.91 (dd, J=7.6, 5.2 Hz, 1H, H2′), 4.75 (t, J=7.2 Hz, 1H, H3′), 3.30-3.23 (m, 1H, H4′), 2.59-2.52 (m, 2H, H5′, under DMSO peak), 1.52 (s, 3H, CH₃, acetonide), 1.21 (s, 3H, CH₃, acetonide). 0.5 eq. of EtOAc and 0.3 eq. of DMF were observed on NMR spectra

### 7-((3αS,4R,6R,6αR)-2,2-dimethyl-6-(3-(2-methyl-1,3-dioxolan-2-yl)phenyl) tetrahydro-4H-cyclopenta[d][1,3]dioxol-4-yl)-7H-pyrrolo[2,3-d]pyrimidin-4-amine (281)

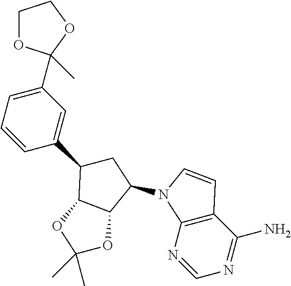 [SMILES: CC1(C)O[C@@H]2[C@H](O1)[C@@H](c1cccc(C3(C)OCCO3)c1)C[C@H]2n1ccc2c(N)ncnc21]

To a solution of the product from previous step (200 mg) in dioxane (4.4 mL) in a pressure flask was added aqueous ammonia (25%, 9 mL). The reaction mixture was stirred for 15 h at 100° C. After concentration, the residue was purified by flash chromatography eluting with DCM/MeOH (0.3-+5.2%) to give 7-((3αS,4R,6R,6αR)-2,2-dimethyl-6-(3-(2-methyl-1,3-dioxolan-2-yl)phenyl) tetrahydro-4H-cyclopenta[d][1,3]dioxol-4-yl)-7H-pyrrolo[2,3-d]pyrimidin-4-amine (yield: 74 mg, 0.17 mmol, 23% over 3 steps), a yellow to brown solid. R^{f}=0.26 (DCM/MeOH 95:5). APCI: calcd. for C₂₄H₂₈N₄O₄ [M+H]⁺: 437.2 found 436.7 ¹H NMR (400 MHZ, DMSO-d₆) δ 8.07 (s, 1H, H2), 7.46 (d, J=3.6 Hz, 1H, H8), 7.43-7.41 (m, 1H, m-H), 7.34-7.27 (m, 3H, o-H, p-H), 6.98 (br s, 2H, NH₂), 6.58 (d, J=3.5 Hz, 1H, H7), 5.15-5.06 (m, 1H, H1′), 4.97 (dd, J=7.6, 5.4 Hz, 1H, H2′), 4.75 (t, J=7.2 Hz, 1H, H3′), 4.00-3.95 (m, 2H, CH₂, ketal), 3.74-3.64 (m, 2H, CH₂, ketal), 3.31-3.22 (m, 1H, H4′), 2.5 (m, 2H, H5′, under DMSO peak), 1.55 (s, 3H, CH₃, ketal), 1.52 (s, 3H, CH₃, acetonide), 1.22 (s, 3H, CH₃, acetonide).

### tert-butyl (4-((3ar,4r,6as)-6-hydroxy-2,2-dimethyltetrahydro-4H-cyclopenta[d][1,3]dioxol-4-yl)phenyl)carbamate (282)

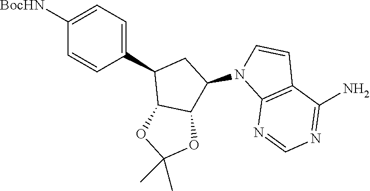 [SMILES: CC(C)(C)OC(=O)Nc1ccc([C@H]2C[C@@H](n3ccc4c(N)ncnc43)[C@@H]3OC(C)(C)O[C@@H]32)cc1]

950 mg (2.72 mmol; 1.00 eq) of tert-butyl (3-((3ar,4r,6as)-6-hydroxy-2,2-dimethyltetrahydro-4H-cyclopenta[d][1,3]dioxol-4-yl)phenyl)carbamate were dissolved in 30 ml of Toluene under inert gas atmosphere. At 0° C., 1427 mg (5.44 mmol; 2.00 eq) of PPh₃ were added. 10 minutes later 1253 mg (5.44 mmol; 2.00 eq) of Di-tert-butyl-azodicarboxylate were attached. The reaction solution was stirred for further 10 minutes before 544 mg (3.54 mmol; 1.30 eq) of 4-chloro-7H pyrrolo[2,3-d]pyrimidine were added and the whole was heated to 80° C. overnight. The solvent was removed under reduced pressure and the crude was purified by column chromatography (Cyclohexane/EtOAc 99:1% to 60:40%). The product was directly used without further characterization. Therefore 20 ml of Dioxane and 20 ml of a 25% aqueous solution of NH₃ were added. The reaction solution was heated to 100° C. in a sealed tube overnight. The solvent was then removed under reduced pressure and the resulting residue was adsorbed on silica and purified by flash chromatography (DCM/MeOH 99%/1% to 90%/10%). 170 mg (0.36 mmol; 13% over 2 steps) of the title compound were obtained in the form of a colorless foam. TLC: R_{f}=0.41 (DCM/MeOH 10:1). 1H-NMR: (400 MHZ, DMSO-d₆) δ=9.30 (s, 1H), 8.08 (s, 1H), 7.48 (s, 1H), 7.43 (d, J=3.6 Hz, 1H), 7.33-7.26 (m, 1H), 7.24-7.20 (m, 1H), 7.03-6.98 (m, 1H), 6.60 (d, J=3.5 Hz, 1H), 5.12-5.06 (m, 1H), 4.99-4.96 (m, 1H), 4.72 (t, J=7.2 Hz, 1H), 3.24-3.14 (m, 2H), 2.46-2.41 (m, 1H), 1.52 (s, 3H), 1.47 (s, 9H), 1.22 (s, 3H) ppm. APCI-MS (+) m/z for C₂₅H₂₉ClN₄O₄: calc.: 484.98; found: 484.6 and 485.5. APCI-MS (+) m/z for C25H31N₅O₄: calc.: 465.55; found: 465.7 and 466.7.

### 7-((3αS,4R,6R,6αR)-6-(3,5-dichlorophenyl)-2,2-dimethyltetrahydro-4H-cyclopenta[d][1,3]dioxol-4-yl)-5-(thiazol-2-yl)-7H-pyrrolo[2,3-d]pyrimidin-4-amine (283)

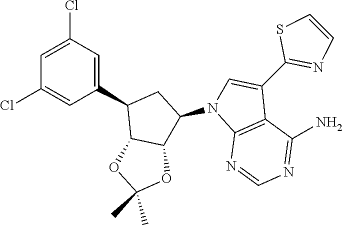

To a solution of compound 265 (0.09 g, 0.17 mmol) in dry toluene (0.01 M, 1.70 mmol) was added Pd (PPh₃) 2Cl₂ (0.04 g, 0.05 mmol) under nitrogen atmosphere, Then, 2-(tributylstanny) thiazole (0.16 mL, 0.51 mmol) was added and the resulted mixture was degassed. The reaction mixture was heated to 120° C. and stirred for 17 h. The mixture was cooled down to ambient temperature and concentrated to complete dryness. The obtained residue was purified over silica gel chromatography (cyclohexane/EtOAc; 0-80%) to afford the title compound as yellowish foam (15 mg, 18%). ¹H NMR (400 MHZ, DMSO-d₆) δ8.40 (s, 1H, H2 adenine), 8.14 (s, 1H, H6 adenine), 7.84 (d, J=3.4 Hz, 1H, H4 thiazole), 7.66 (d, J=3.4 Hz, 1H, H5 thiazole), 7.54-7.51 (m, 3H, o,p Ar), 5.25 (dt, J=12.1, 6.1 Hz, 1H, H1′), 4.96 (dd, J=7.6, 5.2 Hz, 1H, H2′), 4.78 (t, J=7.2 Hz, 1H, H3′), 3.41-3.35 (m, 1H, H4′), 2.63-2.50 (m, 2H, H5′), 1.54 (s, 3H, CH₃, acetonide), 1.24 (s, 3H, CH₃, acetonide).

### 7-((3αS,4R,6R,6αR)-2,2-dimethyl-6-phenyltetrahydro-4//-cyclopenta[d][1,3]dioxol-4-yl)-5-(thiazol-2-yl)-7H-pyrrolo[2,3-a]pyrimidin-4-amine (284)

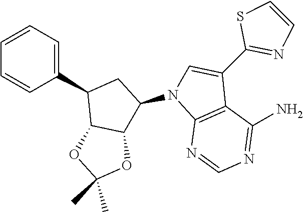

A heat-dried flask was charged with compound 267 (0.09 g, 0.21 mmol) and Pd (PPh₃) 2Cl₂ (0.04 g, 0.06 mmol) under nitrogen atmosphere. Then, dry toluene (0.01 M, 2.10 mmol) was added followed by 2-(tributylstannyl) thiazole (0.20 mL, 0.62 mmol). The resulted mixture was degassed before heated to 120° C. The reaction mixture was refluxed for 17 h before cooled down to ambient temperature. The mixture was concentrated to complet dryness over vacuo and the obtained residue was purified by flash chromatography (cyclohexane/EtOAc; 0-80%) to afford the desired product as yellowish foam (23 mg, 25.6%). R^{f}=0.33 (cyclohexane/EtOAc; 50%). 1H NMR (400 MHZ, DMSO-d₆) δ8.41 (s, 1H, H2), 8.14 (s, 1H, H6), 7.84 (d, J=3.4 Hz, 1H, H4 thiazole), 7.65 (d, J=3.4 Hz, 1H, H5 thiazole), 7.45-7.34 (m, 4H, o, m-Ar), 7.30-7.24 (m, 1H, p-Ar), 5.24 (dt, J=12.1, 6.3 Hz, 1H, H1′), 5.02 (dd, J=7.6, 5.4 Hz, 1H, H2′), 4.77 (t, J=7.2 Hz, 1H, H3′), 3.29 (dt, J=13.0, 6.8 Hz, 1H, H4′), 2.63-2.52 (m, 2H, H5′), 1.54 (s, 3H, CH₃, acetonide), 1.24 (s, 3H, CH₃, acetonide).

### 7-((3αS,4R,6R,6αR)-2,2-dimethyl-6-phenyltetrahydro-4//-cyclopenta[d][1,3]dioxol-4-yl)-5-vinyl-7H-pyrrolo[2,3-d]pyrimidin-4-amine (285)

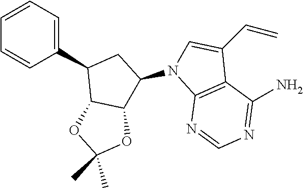

A heat-dried two-round bottom flask was charged with compound 267 (0.10 g, 0.23 mmol), Pd (dtbpf) Cl₂ (0.01 g, 0.01 mmol), 4,4,5,5-tetramethyl-2-vinyl-1,3,2-dioxaborolane (0.05 mL, 0.28 mmol), and K₃PO₄ (0.10 g, 0.46 mmol) under nitrogen atmosphere. Then, 1,4-dioxane (0.10 M, 2.30 mmol) was added and the mixture was degassed. Afterwards, degassed water (1.50 M, 0.20 mmol) was added and the reaction mixture was placed into a pre-heated heating plate (50° C.). The reaction mixture was then heated to 80° C. and stirred for 17 h at this temperature. After cooling down to ambient temperature, the mixture was diluted with water and the aqueous phase was extracted with EtOAc. The combined organic layers were washed with brine, dried over sodium sulfate, and concentrated under reduced pressure. The crude product was purified over silica (cyclohexane/EtOAc; 0-100%) to afford the title product as white foam (17 mg, 20%, 50 mg starting material recovered).

### 7-((3αS,4R,6R,6αR)-2,2-dimethyl-6-phenyltetrahydro-4//-cyclopenta[d][1,3]dioxol-4-yl)-5-ethyl-7H-pyrrolo[2,3-d]pyrimidin-4-amine (286)

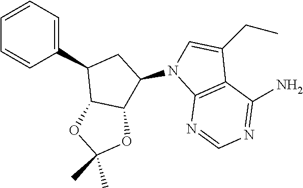

Compound 285 (0.02 g, 0.05 mmol) was dissolved in a mixture of EtOAC/MeOH (1:1, 0.01 M, 4.50 mL) under nitrogen atmosphere. Then, palladium on activated charcoal moistened with water (0.01 g, 10 mol %) was added and the mixture was degassed. The reaction mixture was then purged with hydrogen (balloon) and stirred upon complete consumption. After 3 h, the reaction mixture was set under nitrogen atmosphere and was filtered over celite. The filter cake was rinsed with MeOH and the filtrate was concentrated under reduced pressure to give a colorless oil. The obtained residue was directly used for the next step.

### Synthesis of final compounds

### tert-butyl (7-((3αS,4R,6R,6αR)-6-(((3-((tert-butoxycarbonyl)(3-phenoxyphenethyl) amino)propyl) amino)methyl)-2,2-dimethyltetrahydro-4H-cyclopenta[d][1,3]dioxol-4-yl)-7H-pyrrolo[2,3-d]pyrimidin-4-yl)(methyl)carbamate (286) & (1R,2S,3R,5R)-3-(4-(methylamino)-7H-pyrrolo[2,3-d]pyrimidin-7-yl)-5-(((3-((3-phenoxyphenethyl) amino)propyl) amino) methyl) cyclopentane-1,2-diol (287)

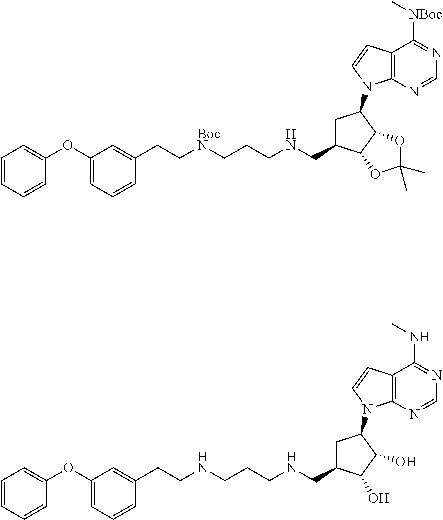 [SMILES: CN(C(=O)OC(C)(C)C)c1ncnc2c1ccn2[C@@H]1C[C@H](CNCCCN(CCc2cccc(Oc3ccccc3)c2)C(=O)OC(C)(C)C)[C@H]2OC(C)(C)O[C@H]21 ; CNc1ncnc2c1ccn2[C@@H]1C[C@H](CNCCCNCCc2cccc(Oc3ccccc3)c2)[C@@H](O)[C@H]1O]

According to general procedure A. Reductive amination (286): yield: 42 mg, 0.071 mmol, 77%. C43H58N₆O₇ (770.97 g/mol). APCI: calc. for C43H58N₆O₇ [M+H]⁺: 770.4, found: 771.5. According to general procedure Q. Deprotection (287): yield: 20.00 mg, 0.037 mmol, 100% (2 TFA salt). C20H34N₈O₅ (466.54 g/mol). The crude product was purified according to method D. yield: 8.15 mg, 0.02 mmol, 42% (2 TFA salt). C30H38N₆O₃ (530.67 g/mol). ¹H NMR (400 MHZ, DMSO-d₆) δ9.57 (bs, 1H, NH), 8.89-8.80 (m, 2H, NH₂+), 8.80-8.67 (m, 2H, NH₂*), 8.36 (s, 1H, H2), 7.66-7.59 (m, 1, H6), 7.43-7.38 (m, 2H, m″), 7.38-7.33 (m, 1H, m′), 7.15 (tt, J=7.2, 1.1 Hz, 1H, p″), 7.06-7.03 (m, 1H, o′), 7.03-6.99 (m, 2H, o″), 6.96 (t, J=2.1 Hz, 1H, o′), 6.93-6.91 (m, 1H, H5), 6.89 (ddd, J=8.1, 2.5, 0.9 Hz, 1H, p′), 4.94 (q, J=8.5 Hz, 1H, H1′), 4.17 (dd, J=7.3, 5.9 Hz, 1H, H2′), 3.88 (t, J=5.3 Hz, 1H, H3′), 3.22-3.11 (m, 2H, CH₂, H4″), 3.12-2.98 (m, 9H, H5′, H1″, H3″, NMe), 2.95-2.87 (m, 2H, CH₂, H5″), 2.37-2.22 (m, 2H, H4′, H6-cyclopentane), 1.97 (p, J=7.8 Hz, 2H, CH₂, H3″), 1.67-1.55 (m, 1H, H6-cyclopentane). APCI: calc. for C30H38N₆O₃ [M+H]⁺: 530.3, found: 530.4. HPLC: tR=13.275 min (Method A). UV-purity at 210 nm=96.4%.

### tert-butyl (7-((3αS,4R,6R,6αR)-6-(((3-((tert-butoxycarbonyl)(3-(methyl(phenyl)amino)phenethyl) amino)propyl) amino)methyl)-2,2-dimethyltetrahydro-4H-cyclopenta[d][1,3]dioxol-4-yl)-7H-pyrrolo[2,3-d]pyrimidin-4-yl)(methyl)carbamate (288) & (1S,2R,3R,5R)-3-(((3-((3-(methyl(phenyl)amino)phenethyl) amino)propyl) amino)methyl)-5-(4-(methylamino)-7H-pyrrolo[2,3-d]pyrimidin-7-yl) cyclopentane-1,2-diol (289)

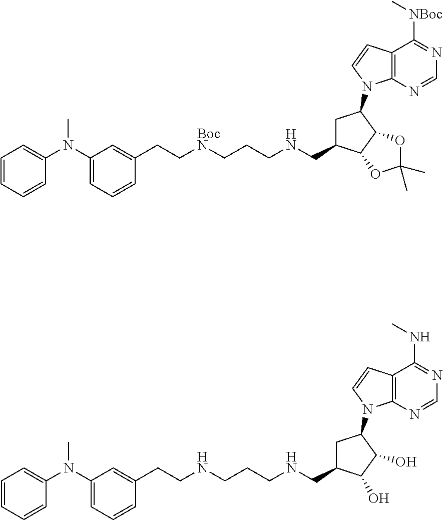 [SMILES: CN(C(=O)OC(C)(C)C)c1ncnc2c1ccn2[C@@H]1C[C@H](CNCCCN(CCc2cccc(N(C)c3ccccc3)c2)C(=O)OC(C)(C)C)[C@H]2OC(C)(C)O[C@H]21 ; CNc1ncnc2c1ccn2[C@@H]1C[C@H](CNCCCNCCc2cccc(N(C)c3ccccc3)c2)[C@@H](O)[C@H]1O]

According to general procedure A. Reductive amination (288): yield: 21.00 mg, 0.03 mmol, 23%. C44H61N₇O₆ (784.02 g/mol). APCI: calc. for C44H61N₇O₆ [M+H]⁺: 784.5, found: 785.2. According to general procedure Q. Deprotection (289): C31H41N₇O₂ (543.72 g/mol). The crude product was purified by preparative HPLC according to method A. yield: 8.00 mg, 0.02 mmol, 42% (2 TFA salt). ¹H NMR (400 MHZ, DMSO-d₆) δ8.77 (s, 2H, NH₂*), 8.70 (s, 2H, NH₂*), 8.32 (s, 1H, H2), 7.55 (s, 1H, H6), 7.32-7.27 (m, 2H, m″), 7.27-7.22 (m, 1H, m′), 7.05-7.01 (m, 2H, o″), 6.98 (tt, J=7.6, 1.1 Hz, 1H, p″), 6.91-6.80 (m, 4H, H5, o′, p′), 4.92 (q, J=8.5 Hz, 1H, H1′), 4.18 (dd, J=7.3, 5.8 Hz, 1H, H2′), 3.92-3.87 (m, 1H, H3′), 3.27 (s, 3H, CH₃, NMe (Ar)), 3.21-3.10 (m, 3H, CH₂, H4″, H5′), 3.11-2.98 (m, 8H, CH₂, CH₃, NMe, 1″, 3″, H5′), 2.90-2.81 (m, 2H CH₂, H5″), 2.38-2.21 (m, 2H, H4′, H6-cyclopentane), 1.97 (p, J=7.9 Hz, 2H CH₂, H2″), 1.67-1.56 (m, 1H, H6-cyclopentane). APCI: calc. for C31H41NO₂ [M+H]⁺: 544.7, found: 545.1. HPLC: tR=13.802 min (Method A). UV-purity at 210 nm=95.7%.

### tert-butyl (7-((3αS,4R,6R,6αR)-6-(((3-((tert-butoxycarbonyl)(phenethyl) amino)propyl) amino)methyl)-2,2-dimethyltetrahydro-4H-cyclopenta[d][1,3]dioxol-4-yl)-7H-pyrrolo[2,3-d]pyrimidin-4-yl)(methyl)carbamate (290) & (1R,2S,3R,5R)-3-(4-(methylamino)-7H-pyrrolo[2,3-a]pyrimidin-7-yl)-5-(((3-(phenethylamino)propyl) amino) methyl) cyclopentane-1,2-diol (291)

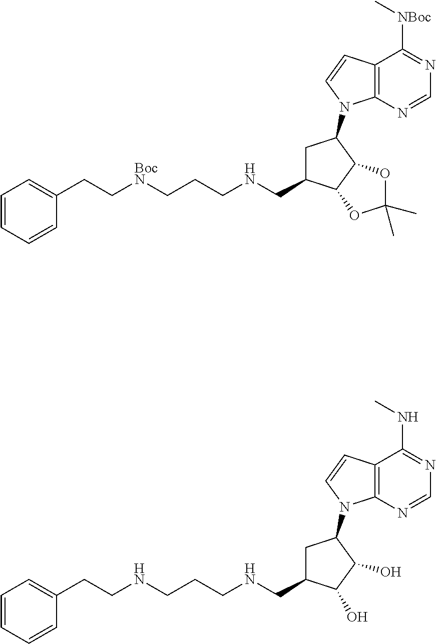 [SMILES: CN(C(=O)OC(C)(C)C)c1ncnc2c1ccn2[C@@H]1C[C@H](CNCCCN(CCc2ccccc2)C(=O)OC(C)(C)C)[C@H]2OC(C)(C)O[C@H]21 ; CNc1ncnc2c1ccn2[C@@H]1C[C@H](CNCCCNCCc2ccccc2)[C@@H](O)[C@H]1O]

According to general procedure A. Reductive amination (290): yield: 32.00 mg, 0.05 mmol, 52%. C₃₇H₅₄N₆O₆ (678.8 g/mol). APCI: calc. for C₃₇H₅₄NO₆ [M+H]⁺: 679.4, found: 679.7. According to general procedure Q. Deprotection (291): C₂₄H₃₄N₆O₂ (438.27 g/mol). The crude product was purified by preparative HPLC according to method A. yield: 12.70 mg, 0.03 mmol, 65% (2 TFA salt). ¹H NMR (400 MHZ, DMSO-d₆) δ 8.94-8.81 (m, 2H, NH₂*), 8.78-8.64 (m, 2H, NH₂+), 8.32 (s, 1H, H2), 7.55 (s, 1H, H6), 7.40-7.32 (m, 2H, m′-Ar), 7.31-7.23 (m, 3H, o′/p′-Ar), 6.84 (s, 1H, H5), 4.92 (q, J=8.6 Hz, 1H, H1′), 4.18 (dd, J=7.3, 5.8 Hz, 1H, H2′), 3.90 (t, J=5.4 Hz, 1H, H3′), 3.24-3.13 (m, 2H, CH₂, H4″), 3.13-3.00 (m, 9H, CH₂ & CH₃, H5′, H1″, H3″, NMe), 2.97-2.89 (m, 2H, CH₂, H5″), 2.39-2.21 (m, 2H, H4′, H6-cyclopentane), 1.99 (p, J=8.0 Hz, 2H, CH₂, H2″), 1.68-1.57 (m, 1H, H6-cyclopentane). APCI: calc. for C24H34NO₂ [M+H]⁺: 439.3, found: 439.4. HPLC: t_{R}=9.836 min (Method A). UV-purity at 210 nm=96.6%.

### tert-butyl (((3αR,4R,6R,6αR)-6-(6-((tert-butoxycarbonyl)amino)-9//purin-9-yl)-2,2-dimethyltetrahydrofuro[3,4-d][1,3]dioxol-4-yl)methyl)(3-((4-((1-(2-methoxyethyl)-1H-1,2,3-triazol-4-yl) methoxy)benzyl) amino)propyl)carbamate (292) & (2R,3R,4S,5R)-2-(6-amino-9H-purin-9-yl)-5-(((3-((4-((1-(2-methoxyethyl)-1/-1,2,3-triazol-4-yl) methoxy)benzyl) amino)propyl) amino) methyl)tetrahydrofuran-3,4-diol (293)

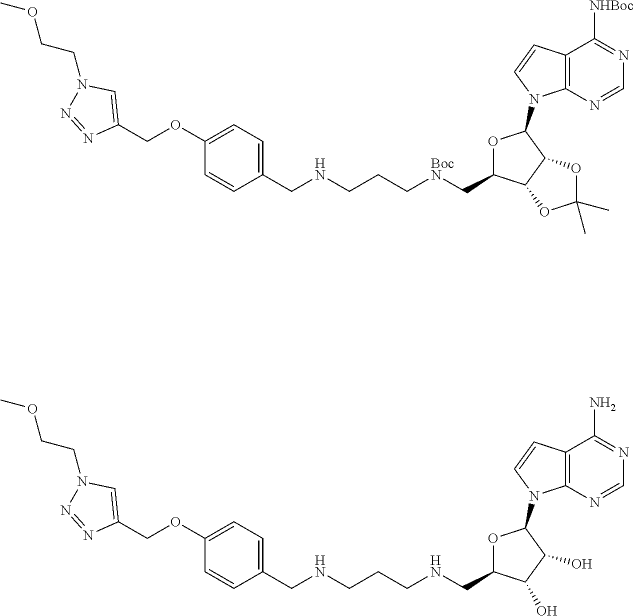 [SMILES: COCCn1cc(COc2ccc(CNCCCN(C[C@H]3O[C@@H](n4ccc5c(NC(=O)OC(C)(C)C)ncnc54)[C@@H]4OC(C)(C)O[C@@H]43)C(=O)OC(C)(C)C)cc2)nn1 ; COCCn1cc(COc2ccc(CNCCCNC[C@H]3O[C@@H](n4ccc5c(N)ncnc54)[C@H](O)[C@@H]3O)cc2)nn1]

According to general procedure G. CuAAC(292): yield: 17.00 mg, 0.02 mmol, 75%. C39H56N₁₀₀₉ (808.94 g/mol). APCI: calc. For C39H56N₁₀₀₉ [M+H]⁺: 809.4, found: 809.5. According to general procedure Q. Deprotection (293): C26H36N₁₀₀₅ (568.64 g/mol). yield: 12.08 mg, 0.02 mmol, quant. (2 TFA salt). ¹H NMR (400 MHZ, DMSO-d₆) δ 8.95-8.62 (m, 4H, NH₂+), 8.47 (s, 1H, H2), 8.29 (s, 1H, H8), 8.21 (s, 1H, H-triazol), 7.92 (s, 2H, N₆-amine), 7.43-7.38 (m, 2H, m′), 7.15-7.10 (m, 2H, o′), 5.99 (d, J=5.2 Hz, 1H, H1′), 5.16 (s, 2H, CH₂, O—CH₂), 4.70 (t, J=5.1 Hz, 1H, H2′), 4.55 (dd, J=5.6, 4.6 Hz, 2H, CH₂, H1′″), 4.27 (t, J=4.5 Hz, 1H, H3′), 4.24-4.18 (m, 1H, H4′), 4.07 (d, J=6.2 Hz, 2H, CH₂, benzylic), 3.74 (dd, J=5.6, 4.7 Hz, 2H, CH₂, H2′″), 3.48-3.30 (m, 2H, CH₂, H5′), 3.25 (s, 3H, CH₃, OMe), 3.07-3.00 (m, 2H, CH₂, H3″), 2.99-2.91 (m, 2H, CH₂, H2″), 1.95 (p, J=6.9 Hz, 2H, CH₂, H2″). APCI: calc. for C26H36N₁₀₀₅ [M+H]⁺: 569.3, found: 569.6. HPLC: t_{R}=8.884 min (Method A). UV-purity at 210 nm=86.8%.

### tert-butyl (((3αR,4R,6R,6αR)-6-(6-((tert-butoxycarbonyl)amino)-9H-purin-9-yl)-2,2-dimethyltetrahydrofuro[3,4-d][1,3]dioxol-4-yl)methyl)(3-((4-(1-(2-methoxyethyl)-1H-1,2,3-triazol-4-yl)phenethyl) amino)propyl)carbamate (294) & (2R,3R,4S,5R)-2-(6-amino-9H-purin-9-yl)-5-(((3-((4-(1-(2-methoxyethyl)-1H-1,2,3-triazol-4-yl)phenethyl) amino)propyl) amino) methyl)tetrahydrofuran-3,4-diol (295)

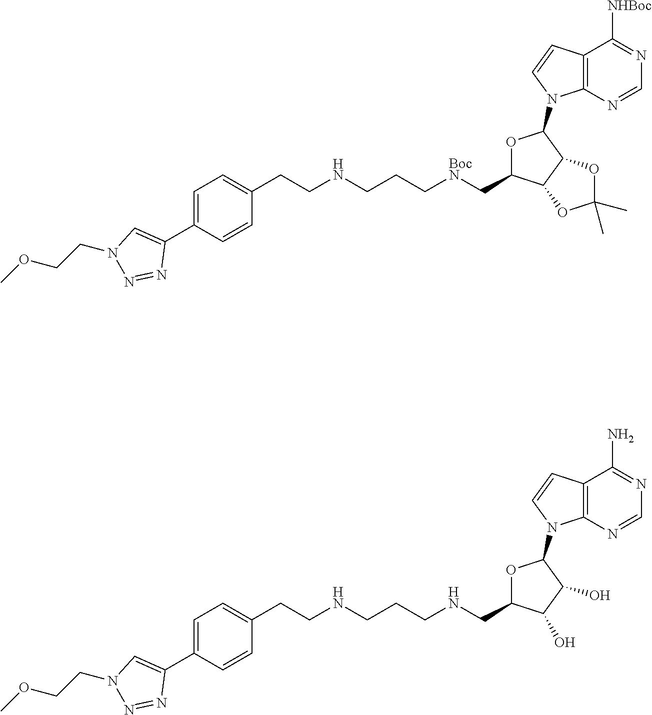 [SMILES: COCCn1cc(-c2ccc(CCNCCCN(C[C@H]3O[C@@H](n4ccc5c(NC(=O)OC(C)(C)C)ncnc54)[C@@H]4OC(C)(C)O[C@@H]43)C(=O)OC(C)(C)C)cc2)nn1 ; COCCn1cc(-c2ccc(CCNCCCNC[C@H]3O[C@@H](n4ccc5c(N)ncnc54)[C@H](O)[C@@H]3O)cc2)nn1]

According to general procedure G. CuAAC(294): yield: 15.00 mg, 0.01 mmol, 19%. C₃₉H₅N₁₀O₈ (792.94 g/mol). APCI: calc. For C₃₉H₅₆N₁₀O₈ [M+H]⁺: 793.4, found: 793.8. According to general procedure Q. Deprotection (295): C₂₆H₃₆N₁₀O₄ (552.64 g/mol). yield: 2.08 mg, 0.004 mmol, quant. (2 TFA salt). ¹H NMR (400 MHZ, DMSO-d₆) δ 8.94-8.66 (m, 4H, NH₂*), 8.54 (s, 1H, H2), 8.44 (s, 1H, H8), 8.26 (s, 1H, H-triazol), 7.87-7.81 (m, 2H, m′), 7.71 (s, 2H, N₆-amine), 7.38-7.32 (m, 2H, o′), 5.99 (d, J=5.3 Hz, 1H, H1′), 4.72 (t, J=5.2 Hz, 1H, H2′), 4.58 (t, J=5.2 Hz, 2H, CH₂, H1′″), 4.28 (t, J=4.6 Hz, 1H, H3′), 4.25-4.19 (m, 1H, H4′), 3.78 (dd, J=5.7, 4.7 Hz, 2H, CH₂, H2′″), 3.49-3.40 (m, 2H, CH₂, H5′), 3.27 (s, 3H, CH₃, OMe), 3.20-3.16 (m, 2H, CH₂, H4″), 3.03 (d, J=6.5 Hz, 4H, CH₂, H1″, H3″), 2.97-2.90 (m, 2H, CH₂, H5″), 2.03-1.90 (m, 2H, CH₂, H2″). APCI: calc. for C₂₆H₃₆N₁₀O₄ [M+H]⁺: 553.3, found 553.5. HPLC: t_{R}=9.449 min (Method A). UV-purity at 210 nm=75.5%.

### tert-butyl (3-((3αS,45,6R,6αR)-6-(6-amino-9H-purin-9-yl)-2,2-dimethyltetrahydrofuro[3,4-d [1,3]dioxole-4-carboxamido)propyl)(4-phenoxyphenethyl)carbamate (296) & (2S,3S,4R,5R)-5-(6-amino-9H-purin-9-yl)-3,4-dihydroxy-N-(3-((4-phenoxyphenethyl) amino)propyl) tetrahydrofuran-2-carboxamide (297)

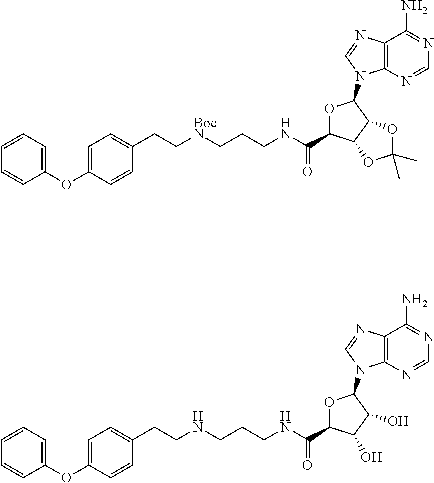 [SMILES: CC(C)(C)OC(=O)N(CCCNC(=O)[C@H]1O[C@@H](n2cnc3c(N)ncnc32)[C@@H]2OC(C)(C)O[C@@H]21)CCc1ccc(Oc2ccccc2)cc1 ; Nc1ncnc2c1ncn2[C@@H]1O[C@H](C(=O)NCCCNCCc2ccc(Oc3ccccc3)cc2)[C@@H](O)[C@H]1O]

According to general procedure E. Amide coupling (296): yield: 67.00 mg, 0.10 mmol, 89%. C₃₅H₄₃N₇O₇ (673.77 g/mol). APCI: calc. C₃₅H₄₃N₇O₇ [M+H]⁺: 674.3, found: 674.4. According to general procedure Q. Deprotection (297): C₂₇H₃₁N₇O₅ (533.59 g/mol). yield: 23.00 mg, 0.04 mmol, 53% (1 TFA salt). ¹H NMR (400 MHZ, DMSO-d₆) δ 9.25 (t, J=6.0 Hz, 1H, NH, amide), 8.53 (s, 2H, NH₂+), 8.39 (s, 1H, H2), 8.27 (s, 1H, H8), 7.51 (s, 2H, NH₂, N₆-amine), 7.43-7.36 (m, 2H, m″), 7.31-7.25 (m, 2H, m′), 7.17-7.11 (m, 1H, p″), 7.02-6.96 (m, 4H, o′, o″), 5.98 (d, J=7.8 Hz, 1H, H1′), 5.87-5.81 (m, 1H, 3′OH), 5.65-5.58 (m, 1H, 2′OH), 4.64-4.57 (m, 1H, H2′), 4.35 (d, J=1.4 Hz, 1H, H4′), 4.17 (d, J=4.6 Hz, 1H, H3′), 3.31 (q, J=6.5 Hz, 2H, CH₂, H1″), 3.21-3.11 (m, 2H, CH₂, H4″), 3.02-2.92 (m, 2H, CH₂, H3″), 2.91-2.84 (m, 2H, CH₂, H5″), 1.83 (p, J=7.0 Hz, 2H, CH₂, H2″). APCI: calc. for C₂₇H₃₁N₇O₅ [M+H]⁺: 534.2, found: 534.3. HPLC: t_{R}=14.071 min (Method A). UV-purity at 210 nm=89.0%.

### tert-butyl (3-((3αS,4S,6R,6αR)-6-(6-amino-9H-purin-9-yl)-2,2-dimethyltetrahydrofuro[3,4-d [1,3]dioxole-4-carboxamido)propyl)(3-phenoxyphenethyl)carbamate (298) & (2S,3S,4R,5R)-5-(6-amino-9H-purin-9-yl)-3,4-dihydroxy-N-(3-((3-phenoxyphenethyl) amino)propyl) tetrahydrofuran-2-carboxamide (299)

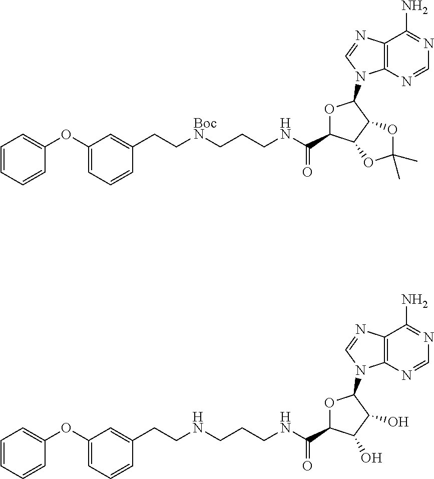 [SMILES: CC(C)(C)OC(=O)N(CCCNC(=O)[C@H]1O[C@@H](n2cnc3c(N)ncnc32)[C@@H]2OC(C)(C)O[C@@H]21)CCc1cccc(Oc2ccccc2)c1 ; Nc1ncnc2c1ncn2[C@@H]1O[C@H](C(=O)NCCCNCCc2cccc(Oc3ccccc3)c2)[C@@H](O)[C@H]1O]

According to general procedure E. Amide coupling (298): yield: 32.00 mg, 0.05 mmol, 39%. C₃₅H₄₃N₇O₇ (673.77 g/mol). APCI: calc. C₃₅H₄₃N₇O₇ [M+H]⁺: 674.3, found: 674.7. According to general procedure Q. Deprotection (299): C₂₇H₃₁N₇O₅ (533.59 g/mol). The crude product was purified by preparative HPLC according to method A. yield: 17.80 mg, 0.03 mmol, 81% (1 TFA salt). ¹H NMR (400 MHZ, DMSO-d₆) δ 8.96 (t, J=6.1 Hz, 1H, NH, amide), 8.59 (s, 1H, H2), 8.48 (s, 2H, NH₂+), 8.38 (s, 1H, H8), 8.25 (s, 2H, NH₂, N₆-amino), 7.43-7.38 (m, 2H, m″), 7.36 (t, J=7.9 Hz, 1H, m′), 7.18-7.13 (m, 1H, p″), 7.06-7.03 (m, 1H, p′), 7.03-7.00 (m, 2H, o″), 6.97-6.95 (m, 1H, o′), 6.92-6.87 (m, 1H, o′), 6.02 (d, J=7.2 Hz, 1H, H1′), 4.60 (dd, J=7.2, 4.5 Hz, 1H, H2′), 4.38 (d, J=1.8 Hz, 1H, H4′), 4.20 (dd, J=4.6, 1.9 Hz, 1H, H3′), 3.27 (q, J=6.6 Hz, 2H, CH₂, H1″), 3.22-3.12 (m, 2H, CH₂, H4″), 3.00-2.92 (m, 2H, CH₂, H3″), 2.92-2.85 (m, 2H, H5″), 1.80 (p, J=6.7 Hz, 2H, CH₂, H2″). APCI: calc. for C₂₇H₃₁N₇O₅ [M+H]⁺: 534.2, found: 534.5. HPLC: t_{R}=13.888 min (Method A). UV-purity at 210 nm=99.7%.

### tert-butyl (3-((3αS,4S,6R,6αR)-6-(6-amino-9H-purin-9-yl)-2,2-dimethyltetrahydrofuro[3,4-d][1,3]dioxole-4-carboxamido)propyl)(phenethyl)carbamate (300) & (2S,3S,4R,5R)-5-(6-amino-9H-purin-9-yl)-3,4-dihydroxy-N-(3-(phenethylamino)propyl) tetrahydrofuran-2-carboxamide (301)

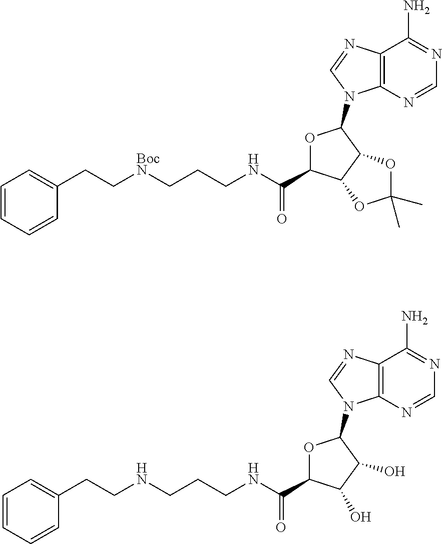 [SMILES: CC(C)(C)OC(=O)N(CCCNC(=O)[C@H]1O[C@@H](n2cnc3c(N)ncnc32)[C@@H]2OC(C)(C)O[C@@H]21)CCc1ccccc1 ; Nc1ncnc2c1ncn2[C@@H]1O[C@H](C(=O)NCCCNCCc2ccccc2)[C@@H](O)[C@H]1O]

According to general procedure E. Amide coupling (300): yield: 20.00 mg, 0.03 mmol, 51%. C₂₉H₃₉N₇O₆ (581.67 g/mol). APCI: calc. C₂₉H₃₉N₇O₆ [M+H]⁺: 582.3, found: 582.6. According to general procedure Q. Deprotection (301): C₂₁H₂₇N₇O₄ (441.49 g/mol). The crude product was purified by preparative HPLC according to method D. yield: 7.80 mg, 0.02 mmol, 83% (1 TFA salt). ¹H NMR (400 MHZ, DMSO-d₆) δ 9.09 (t, J=5.9 Hz, 1H, NH, amide), 8.50 (s, 3H, H2, NH₂+), 8.33 (s, 1H, H8), 7.92 (bs, 2H, NH₂, N₆-amine), 7.39-7.31 (m, 3H, m/p-Ar), 7.31-7.22 (m, 2H, o-Ar), 6.00 (d, J=7.4 Hz, 1H, H1′), 4.61 (dd, J=7.5, 4.6 Hz, 1H, H2′), 4.37 (d, J=1.7 Hz, 1H, H4′), 4.19 (dd, J=4.6, 1.7 Hz, 1H, H3′), 3.29 (q, J=6.6 Hz, 2H, CH₂, H1″), 3.22-3.12 (m, 2H, CH₂, H4′″), 3.02-2.92 (m, 2H, CH₂, H3″), 2.92-2.86 (m, 2H, CH₂, H5″), 1.82 (p, J=6.7 Hz, 2H, CH₂, H2″). APCI: calc. for C₂₁H₂₇N₇O₄ [M+H]⁺: 442.2, found: 442.2. HPLC: t_{R}=10.084 min (Method A). UV-purity at 210 nm =98.8%.

### (3αS,4S,6R,6αR)-6-(6-amino-9H-purin-9-yl)-2,2-dimethyl-N-(3-(2-phenylacetamido)propyl) tetrahydrofuro[3,4-d][1,3]dioxole-4-carboxamide (302) & 2S,3S,4R,5R)-5-(6-amino-9H-purin-9-yl)-3,4-dihydroxy-N-(3-(2-phenylacetamido)propyl) tetrahydrofuran-2-carboxamide (303)

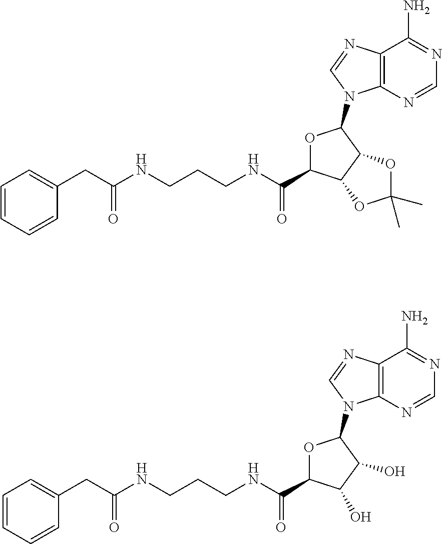 [SMILES: CC1(C)O[C@@H]2[C@H](O1)[C@@H](C(=O)NCCCNC(=O)Cc1ccccc1)O[C@H]2n1cnc2c(N)ncnc21 ; Nc1ncnc2c1ncn2[C@@H]1O[C@H](C(=O)NCCCNC(=O)Cc2ccccc2)[C@@H](O)[C@H]1O]

According to general procedure E. Amide coupling (302): yield: 136.00 mg, 0.27 mmol, 53%. C₂₄H₂₉N₇O₅ (495.54 g/mol). APCI: calc. C₂₄H₂₉N₇O₅ [M+H]⁺: 496.2, found: 496.1. According to general procedure Q. Deprotection (303): C21H25N₇O₅ (455.48 g/mol). The crude product was purified by preparative HPLC according to method D. yield: 84.90 mg, 0.19 mmol, 93% (1 TFA salt). ¹H NMR (400 MHZ, DMSO-d₆) δ 8.70 (s, 1H, H2), 8.62 (t, J=5.7 Hz, 1H, NH, amide), 8.40 (s, 1H, H8), 8.07 (t, J=5.6 Hz, 1H, NH, amide), 7.33-7.17 (m, 5H, Ar), 6.03 (d, J=7.0 Hz, 1H, H1′), 4.61 (dd, J=7.0, 4.5 Hz, 1H, H2′), 4.37 (d, J=2.1 Hz, 1H, H4′), 4.19 (dd, J=4.5, 2.1 Hz, 1H, H3′), 3.39 (s, 2H, CH₂, benzylic), 3.21-3.12 (m, 2H, CH₂, H3″), 3.07 (q, J=6.5 Hz, 2H, CH₂, H1″), 1.60 (p, J=7.0 Hz, 2H, CH₂, H2″). APCI: calc. for C21H25N₇O₅ [M+H]⁺: 456.2, found: 456.4. HPLC: TR=10.899 min (Method A). UV-purity at 210 nm=98.7%.

### (3αS,4S,6R,6αR)-6-(6-amino-9H-purin-9-yl)-2,2-dimethyl-N-(3-(3-phenylureido)propyl) tetrahydrofuro[3,4-d][1,3]dioxole-4-carboxamide (683) & (2S,3S,4R,5R)-5-(6-amino-9H-purin-9-yl)-3,4-dihydroxy-N-(3-(3-phenylureido)propyl) tetrahydrofuran-2-carboxamide (684)

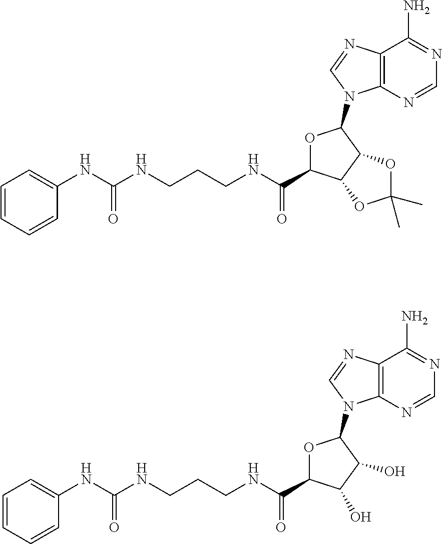 [SMILES: CC1(C)O[C@@H]2[C@H](O1)[C@@H](C(=O)NCCCNC(=O)Nc1ccccc1)O[C@H]2n1cnc2c(N)ncnc21 ; Nc1ncnc2c1ncn2[C@@H]1O[C@H](C(=O)NCCCNC(=O)Nc2ccccc2)[C@@H](O)[C@H]1O]

According to general procedure E. Amide coupling (683): yield: 44.00 mg, 0.09 mmol, 17%. C₂₃H₂₈N₈O₅ (496.53 g/mol). APCI: calc. [M+H]⁺: 497.2, found: 497.1. According to general procedure Q. Deprotection (684): C₂₀H₂₄N₇O₅ (456.46 g/mol). The crude product was purified by preparative HPLC according to method D. yield: 13.30 mg, 0.03 mmol, 37% (1 TFA salt). ¹H NMR (400 MHZ, DMSO-d₆) δ 8.70 (s, 1H, H2), 8.63 (t, J=5.7 Hz, 1H, NH, amide), 8.51 (s, 1H, NH, urea), 8.41 (s, 1H, H8), 7.39-7.34 (m, 2H, o′), 7.23-7.16 (m, 2H, m′), 6.87 (tt, J=7.4, 1.2 Hz, 1H, p′), 6.24-6.16 (m, 1H, NH, urea), 6.03 (d, J=7.0 Hz, 1H, H1′), 4.62 (dd, J=6.9, 4.5 Hz, 1H, H2′), 4.38 (d, J=2.1 Hz, 1H, H4′), 4.21 (dd, J=4.6, 2.1 Hz, 1H, H3′), 3.21 (p, J=6.8 Hz, 2H, CH₂, H1″), 3.13-3.05 (m, 2H, CH₂, H3″), 1.61 (p, J=6.8 Hz, 2H, CH₂, H2″). APCI: calc. for C20H24N₈O₅ [M+H]⁺: 457.2, found: 457.4. HPLC: t_{R}=10.460 min (Method A). UV-purity at 210 nm=95.9%.

### (3αS,4S,6R,6αR)-6-(6-amino-9H-purin-9-yl)-2,2-dimethyl-N-(3-propionamidopropyl) tetrahydrofuro[3,4-d][1,3]dioxole-4-carboxamide (304) & (2,3S,4R,5R)-5-(6-amino-9//-purin-9-yl)-3,4-dihydroxy-N-(3-propionamidopropyl) tetrahydrofuran-2-carboxamide (305)

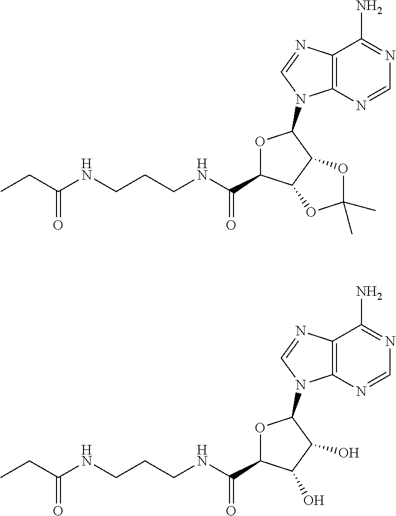 [SMILES: CCC(=O)NCCCNC(=O)[C@H]1O[C@@H](n2cnc3c(N)ncnc32)[C@@H]2OC(C)(C)O[C@@H]21 ; CCC(=O)NCCCNC(=O)[C@H]1O[C@@H](n2cnc3c(N)ncnc32)[C@H](O)[C@@H]1O]

According to general procedure E. Amide coupling (304): yield: 36.00 mg, 0.08 mmol, 20%. C19H27N₇O₅ (433.47 g/mol). APCI: calc. [M+H]⁺: 434.2, found: 434.1. According to general procedure Q. Deprotection (305): C₁₆H₂₃N₇O₅ (448.48 g/mol). The crude product was purified by preparative HPLC according. yield: 33.02 mg, 0.08 mmol, 100% (1 TFA salt). ¹H NMR (400 MHZ, DMSO-d₆) δ 8.75 (s, 1H, H2), 8.53 (t, J=5.8 Hz, 1H, NH, amide), 8.44 (s, 1H, H8), 7.76 (t, J=5.6 Hz, 1H, NH, amide), 6.04 (d, J=6.8 Hz, 1H, H1′), 4.62 (dd, J=6.9, 4.5 Hz, 1H, H2′), 4.37 (d, J=2.2 Hz, 1H, H4′), 4.21 (dd, J=4.6, 2.2 Hz, 1H, H3′), 3.16 (q, J=6.8 Hz, 2H, CH₂, H1″), 3.05 (q, J=6.7 Hz, 2H, CH₂, H3″), 2.05 (q, J=7.6 Hz, 2H, CH₂, H·), 1.58 (p, J=6.9 Hz, 2H, CH₂, H2″), 0.98 (t, J=7.6 Hz, 3H, CH₃, HB). APCI: calc. for C₁₆H₂₃N₇O₅ [M+H]⁺: 394.2, found: 393.8. HPLC: t_{R}=4.881 min (Method B). UV-purity at 210 nm=97.1%.

### (3αS,4S,6R,6αR)-6-(6-amino-9H-purin-9-yl)-N-(3-(3-ethylureido)propyl)-2,2-dimethyltetrahydrofuro[3,4-d][1,3]dioxole-4-carboxamide (306) & (2S,3S,4R,5R)-5-(6-amino-9H-purin-9-yl)-N-(3-(3-ethylureido)propyl)-3,4-dihydroxytetrahydrofuran-2-carboxamide (307)

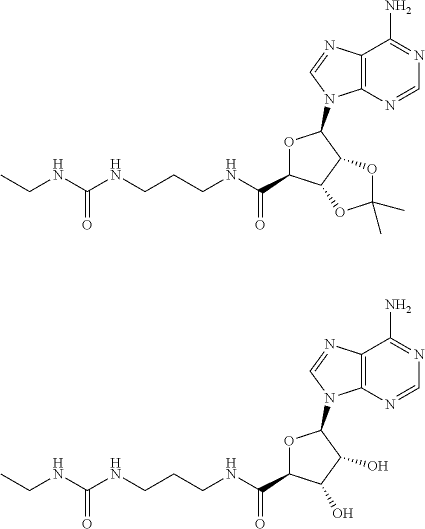 [SMILES: CCNC(=O)NCCCNC(=O)[C@H]1O[C@@H](n2cnc3c(N)ncnc32)[C@@H]2OC(C)(C)O[C@@H]21 ; CCNC(=O)NCCCNC(=O)[C@H]1O[C@@H](n2cnc3c(N)ncnc32)[C@H](O)[C@@H]1O]

According to general procedure E. Amide coupling (306): yield: 46.00 mg, 0.10 mmol, 27%. C₁₉H₂₈N₈O₅ (448.48 g/mol). APCI: calc. [M+H]⁺: 449.2, found: 448.9. According to general procedure Q. Deprotection (307): C₁₆H₂₄N₇O₅ (408.42 g/mol). The crude product was purified by preparative HPLC according to method E. yield: 46.00 mg, 0.11 mmol, 100% (1 TFA salt). ¹H NMR (400 MHZ, DMSO-d₆) δ 8.71 (s, 1H, H2), 8.60 (t, J=5.9 Hz, 1H, NH, amide), 8.40 (s, 1H, H8), 6.03 (d, J=6.9 Hz, 1H, H1′), 4.61 (dd, J=6.9, 4.5 Hz, 1H, H2′), 4.37 (d, J=2.1 Hz, 1H, H4′), 4.20 (dd, J=4.6, 2.2 Hz, 1H, H3′), 3.16 (qd, J=6.7, 2.8 Hz, 2H, CH₂, H1″), 2.98 (q, J=7.1 Hz, 4H, CH₂, H3″, H·-urea), 1.54 (p, J=6.9 Hz, 2H, CH₂, H2″), 0.97 (t, J=7.2 Hz, 3H, CH₃, H·-urea). APCI: calc. for C₁₆H₂₄N₇O₅ [M+H]⁺: 408.2, found: 408.9. HPLC: t_{R}=4.284 min (Method B). UV-purity at 210 nm=100%.

### (3αS,45,6R,6αR)-6-(6-amino-9H-purin-9-yl)-N-(3-(3-ethylureido)propyl)-2,2-dimethyltetrahydrofuro[3,4-d][1,3]dioxole-4-carboxamide (685) & (2S,3S,4R,5R)-5-(6-amino-9H-purin-9-yl)-N-(3-(3-ethylureido)propyl)-3,4-dihydroxytetrahydrofuran-2-carboxamide (686)

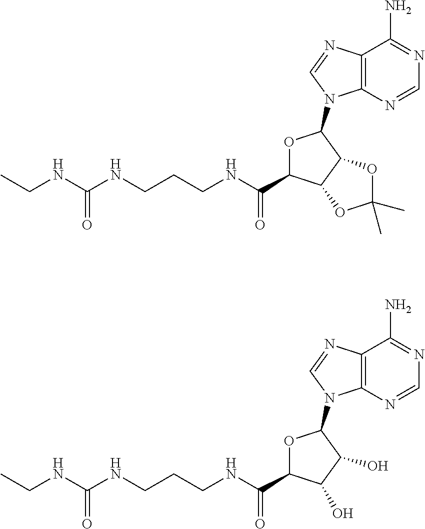 [SMILES: CCNC(=O)NCCCNC(=O)[C@H]1O[C@@H](n2cnc3c(N)ncnc32)[C@@H]2OC(C)(C)O[C@@H]21 ; CCNC(=O)NCCCNC(=O)[C@H]1O[C@@H](n2cnc3c(N)ncnc32)[C@H](O)[C@@H]1O]

According to general procedure E. Amide coupling (685): yield: 46.00 mg, 0.10 mmol, 27%. C19H28N₈O₅ (448.48 g/mol). APCI: calc. [M+H]⁺: 449.2, found: 448.9. According to general procedure Q. Deprotection (686): C16H24N₈O₅ (408.42 g/mol). The crude product was purified by preparative HPLC according to method E. yield: 46.00 mg, 0.11 mmol, 100% (1 TFA salt). ¹H NMR (400 MHZ, DMSO-d₆) δ 8.71 (s, 1H, H2), 8.60 (t, J=5.9 Hz, 1H, NH, amide), 8.40 (s, 1H, H8), 6.03 (d, J=6.9 Hz, 1H, H1′), 4.61 (dd, J=6.9, 4.5 Hz, 1H, H2′), 4.37 (d, J=2.1 Hz, 1H, H4′), 4.20 (dd, J=4.6, 2.2 Hz, 1H, H3′), 3.16 (qd, J=6.7, 2.8 Hz, 2H, CH₂, H1″), 2.98 (q, J=7.1 Hz, 4H, CH₂, H3″, H·-urea), 1.54 (p, J=6.9 Hz, 2H, CH₂, H2″), 0.97 (t, J=7.2 Hz, 3H, CH₃, H·-urea). APCI: calc. for C16H24N₇O₅ [M+H]⁺: 408.2, found: 408.9. HPLC: t_{R}=4.284 min (Method B). UV-purity at 210 nm=100%.

### tert-butyl (7-((3αR,4R,6R,6αR)-6-(((3-((tert-butoxycarbonyl)(phenethyl) amino)propyl) amino)methyl)-2,2-dimethyltetrahydrofuro[3,4-ď [1,3]dioxol-4-yl)-7H-pyrrolo[2,3-d]pyrimidin-4-yl)(methyl)carbamate (308) & (2R,3R,45,5R)-2-(4-(methylamino)-7H-pyrrolo[2,3-d]pyrimidin-7-yl)-5-(((3-(phenethylamino) pronyl) amino) methyl) tetrahydrofuran-3.4-diol (309)

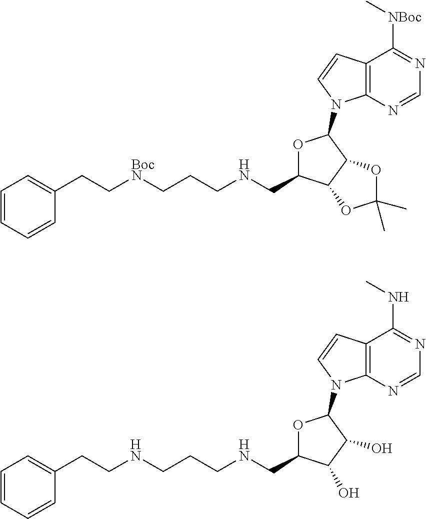 [SMILES: CN(C(=O)OC(C)(C)C)c1ncnc2c1ccn2[C@@H]1O[C@H](CNCCCN(CCc2ccccc2)C(=O)OC(C)(C)C)[C@H]2OC(C)(C)O[C@H]21 ; CNc1ncnc2c1ccn2[C@@H]1O[C@H](CNCCCNCCc2ccccc2)[C@@H](O)[C@H]1O]

According to general procedure A. Reductive amination (308): yield: 44.00 mg, 0.07 mmol, 27%. C₃₆H₅₂N₆O₇ (680.85 g/mol). APCI: calc. [M+H]⁺: 680.4, found: 680.6. According to general procedure Q. Deprotection (309): C₂₃H₃₂N₇O₃ (440.55 g/mol). The crude product was purified by preparative HPLC according to method D. yield: 27.00 mg, 0.06 mmol, 100% (1 TFA salt). ¹H NMR (400 MHZ, DMSO-d₆) δ 8.93 (s, 2H, NH₂+), 8.82 (s, 2H, NH₂+), 8.34 (s, 1H, H2), 7.57 (s, 1H, H6), 7.38-7.32 (m, 2H, o′), 7.30-7.24 (m, 3H, m′/p′), 6.84 (s, 1H, H5), 6.12 (d, J=5.1 Hz, 1H, H1′), 4.47 (t, J=4.9 Hz, 1H, H2′), 4.21-4.13 (m, 2H, H3′, H4′), 3.41-3.28 (m, 2H, CH₂, H5′), 3.15 (d, J=6.0 Hz, 2H, CH₂, H4″), 3.10-2.97 (m, 7H, CH₃, CH₂, NMe, H1″, H3″), 2.95-2.87 (m, 2H, CH₂, H5″), 2.01-1.91 (m, 2H, CH₂, H2″). APCI: calc. for C₂₃H₃₂N₆O₃ [M+H]⁺: 440.3, found: 440.5. HPLC: t_{R}=8.816 min (Method A). UV-purity at 210 nm=98.3%.

### tert-butyl (7-((3αR,4R,6R,6αR)-6-(((3-((tert-butoxycarbonyl)(phenethyl) amino)propyl) amino)methyl)-2,2-dimethyltetrahydrofuro[3,4-d][1,3]dioxol-4-yl)-5-methyl-7H-pyrrolo[2,3-a]pyrimidin-4-yl)(methyl)carbamate (310) & (2R,3R,45,5R)-2-(5-methyl-4-(methylamino)-7//-pyrrolo[2,3-a]pyrimidin-7-yl)-5-(((3-(phenethylamino)propyl) amino) methyl)tetrahydrofuran-3,4-diol (311)

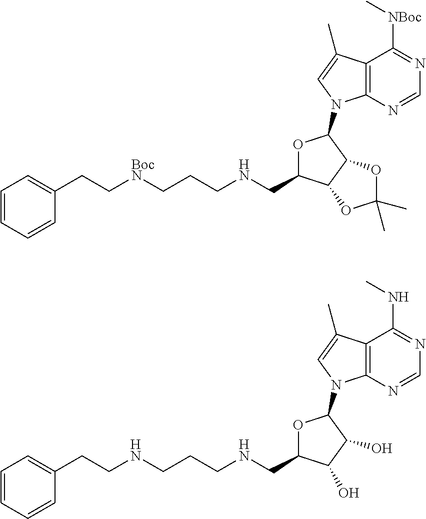 [SMILES: Cc1cn([C@@H]2O[C@H](CNCCCN(CCc3ccccc3)C(=O)OC(C)(C)C)[C@H]3OC(C)(C)O[C@H]32)c2ncnc(N(C)C(=O)OC(C)(C)C)c12 ; CNc1ncnc2c1c(C)cn2[C@@H]1O[C@H](CNCCCNCCc2ccccc2)[C@@H](O)[C@H]1O]

According to general procedure D. Reductive amination (310): yield: 98.00 mg, 0.14 mmol, 62%. C₃₇H₅₄N₆O₇ (694.87 g/mol). APCI: calc. [M+H]⁺: 694.4, found: 694.8. According to general procedure Q. Deprotection (311): C₂₄H₃₄N₆O₃ (454.58 g/mol). The crude product was purified by preparative HPLC according to method D. yield: 6.87 mg, 0.02 mmol, 70% (2 TFA salt). ¹H NMR (400 MHZ, DMSO-d₆) δ 8.84 (bs, 2H, NH₂+), 8.74 (bs, 2H, NH₂+), 8.26 (s, 1H, H2), 7.39-7.32 (m, 2H, o′), 7.31-7.23 (m, 4H, m′, p′, H6), 6.09 (d, J=5.0 Hz, 1H, H1′), 4.40 (t, J=4.8 Hz, 1H, H2′), 4.16-4.10 (m, 2H, H3′, H4′), 3.39-3.24 (m, 2H, CH₂, H5′), 3.20-3.10 (m, 2H, CH₂, H4″), 3.06-2.97 (m, 4H, CH₂, H1″, H3″), 3.02 (d, J=4.6 Hz, 3H, CH₃, NMe), 2.94-2.86 (m, 2H, CH₂, H5″), 2.39 (d, J=1.2 Hz, 3H, CH₃, 5′-Me), 2.00-1.89 (m, 2H, CH₂, H2″). APCI: calc. for C24H34N₆O₃ [M+H]⁺: 454.3, found: 454.5. HPLC: t_{R}=9.136 min (Method A). UV-purity at 210 nm=98.0%.

### tert-butyl (((3αR,4R,6R,6αR)-6-(6-((tert-butoxycarbonyl)amino)-9//purin-9-yl)-2,2-dimethyltetrahydrofuro[3,4-d][1,3]dioxol-4-yl)methyl)(3-propionamidopropyl)carbamate (312) & N-(3-((((2R,3S,4R,5R)-5-(6-amino-9//-purin-9-yl)-3,4-dihydroxytetrahydrofuran-2-yl)methyl) amino)propyl)propionamide (313)

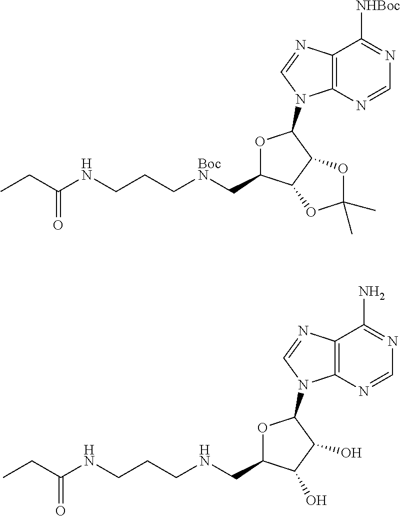 [SMILES: CCC(=O)NCCCN(C[C@H]1O[C@@H](n2cnc3c(NC(=O)OC(C)(C)C)ncnc32)[C@@H]2OC(C)(C)O[C@@H]21)C(=O)OC(C)(C)C ; CCC(=O)NCCCNC[C@H]1O[C@@H](n2cnc3c(N)ncnc32)[C@H](O)[C@@H]1O]

According to general procedure C. Acylation/urea synthesis (312): yield: 8.00 mg, 0.01 mmol, 26%. C₂₉H₄₅N₇O₈ (619.72 g/mol). APCI: calc. [M+H]⁺: 619.3, found: 619.5. According to general procedure Q. Deprotection (313): C₁₆H₂₅NO₄ (379.42 g/mol). yield: 8.61 mg, 0.02 mmol, quant. (1 TFA salt). ¹H NMR (400 MHZ, DMSO-d₆) δ8.84-8.56 (m, 2H, NH₂+), 8.60 (s, 1H, H2), 8.40 (s, 1H, H8), 8.35 (s, 2H, N₆—NH₂), 8.00 (t, J=5.8 Hz, 1H, NH, amide), 6.06 (d, J=5.5 Hz, 1H, H1′), 4.81-4.71 (m, 1H, H2′), 4.32-4.23 (m, 2H, H3′, H4′), 3.54-3.34 (m, 2H, CH₂, H5′), 3.14 (q, J=6.5 Hz, 2H, CH₂, H3″), 2.99 (p, J=7.1 Hz, 2H, CH₂, H1″), 2.12 (q, J=7.6 Hz, 2H, CH₂, Ha), 1.77 (dq, J=13.3, 6.6 Hz, 2H, CH₂, H2″), 1.04 (t, J=7.6 Hz, 3H, CH₃, HB). APCI: calc. for C₁₆H₂₅N₇O₄ [M+H]⁺: 379.2, found: 379.3. HPLC: t_{R}=3.063 min (Method B). UV-purity at 210 nm=91.8%.

### tert-butyl (((3αR,4R,6R,6αR)-6-(6-((tert-butoxycarbonyl)amino)-9//-purin-9-yl)-2,2-dimethyltetrahydrofuro[3,4-d][1,3]dioxol-4-yl)methyl)(2-propionamidoethyl)carbamate (314) & N-(2-((((2R,3S,4R,5R)-5-(6-amino-9H-purin-9-yl)-3,4-dihydroxytetrahydrofuran-2-yl)methyl) amino) ethyl)propionamide (315)

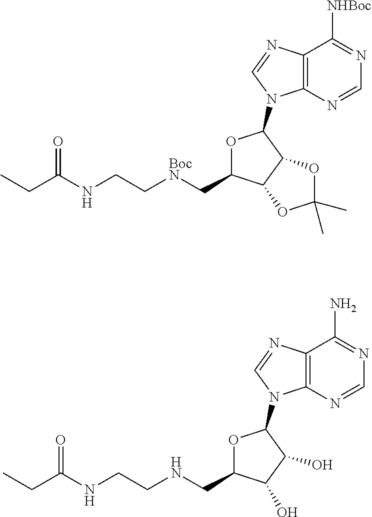 [SMILES: CCC(=O)NCCN(C[C@H]1O[C@@H](n2cnc3c(NC(=O)OC(C)(C)C)ncnc32)[C@@H]2OC(C)(C)O[C@@H]21)C(=O)OC(C)(C)C ; CCC(=O)NCCNC[C@H]1O[C@@H](n2cnc3c(N)ncnc32)[C@H](O)[C@@H]1O]

According to general procedure C. Acylation/urea synthesis (314): yield: 13.00 mg, 0.02 mmol, 17%.

C₂₈H₄₃N₇O₈ (605.69 g/mol). APCI: calc. [M+H]⁺: 605.3, found: 605.5. According to general procedure Q. Deprotection (315): C₁₅H₂₃N₇O₄ (365.39 g/mol). yield: 8.33 mg, 0.02 mmol, 100% (1 TFA salt). ¹H NMR (400 MHZ, DMSO-d₆) δ8.80-8.53 (m, 2H, NH₂+), 8.50 (s, 1H, H2), 8.31 (s, 1H, H8), 8.09 (s, 2H, N₆-amine), 8.00 (t, J=5.7 Hz, 1H, NH, amide), 5.99 (d, J=5.6 Hz, 1H, H1′), 4.69 (dd, J=5.5, 4.4 Hz, 1H, H2′), 4.26-4.18 (m, 2H, H3′, H4′), 3.50-3.36 (m, 2H, CH₂, H5′), 3.32 (q, J=6.3 Hz, 2H, CH₂, H2″), 3.03 (t, J=6.2 Hz, 2H, CH₂, H1″), 2.07 (q, J=7.5 Hz, 2H, CH₂, Ha), 0.96 (t, J=7.6 Hz, 3H, CH₃, HB). APCI: calc. for C₁₅H₂₃N₇O₄ [M+H]⁺: 365.2, found: 366.1. HPLC: t_{R}=6.431 min (Method B). UV-purity at 210 nm=86.0%.

### tert-butyl (((3αR,4R,6R,6αS)-6-(5-bromo-4-((4-methoxybenzyl)amino)-7H-pyrrolo[2,3-d]pyrimidin-7-yl)-2,2-dimethyltetrahydro-4H-cyclopenta[d][1,3]dioxol-4-yl)methyl)(3-((3-fluorophenethyl) amino)propyl)carbamate (316) & (1R,2S,3R,5R)-3-(4-amino-5-bromo-7H-pyrrolo[2,3-d]pyrimidin-7-yl)-5-(((3-((3-fluorophenethyl) amino)propyl) amino) methyl) cyclopentane-1,2-diol (317)

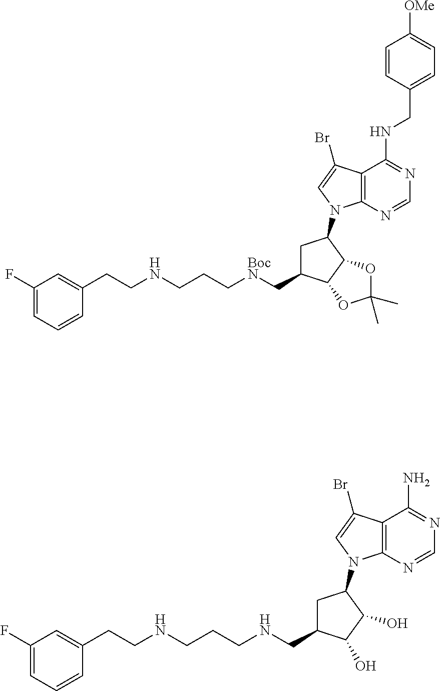 [SMILES: COc1ccc(CNc2ncnc3c2c(Br)cn3[C@@H]2C[C@H](CN(CCCNCCc3cccc(F)c3)C(=O)OC(C)(C)C)[C@H]3OC(C)(C)O[C@H]32)cc1 ; Nc1ncnc2c1c(Br)cn2[C@@H]1C[C@H](CNCCCNCCc2cccc(F)c2)[C@@H](O)[C@H]1O]

According to general procedure D. Reductive amination (316): yield: 20.00 mg, 0.03 mmol, 43%. C₃₉H₅₀BrFN₆O₅ (781.77 g/mol). APCI: calc. [M+H]⁺: 780.30, found:. According to general procedure O. Deprotection (317): C₂₃H₃₀BrFN₆O₂ (521.44 g/mol). The crude product was purified by preparative HPLC according to method D. yield: 12.61 mg, 0.02 mmol, 100% (2 TFA salt). 1H NMR (400 MHZ, DMSO-d₆) δ 8.81 (bs, 2H, NH₂+), 8.65 (bs, 2H, NH₂+), 8.30 (s, 1H, H2), 7.83 (s, 1H, H6), 7.44-7.37 (m, 1H, m′), 7.19-7.09 (m, 3H, o′, p′), 4.95 (dt, J=10.4, 7.9 Hz, 1H, H1′), 4.18 (dd, J=7.6, 5.7 Hz, 1H, H2′), 3.88 (t, J=5.2 Hz, 1H, H3′), 3.26-3.14 (m, 2H, CH₂, H5′, H4″), 3.10-2.99 (m, 5H, CH₂, H5′, H1″, H2″), 2.99-2.91 (m, 2H, CH₂, H5″), 2.36-2.21 (m, 2H, H4′, H6′-cyclopentane-axial), 2.04-1.92 (m, 2H, CH₂, H2″), 1.66-1.54 (m, 1H, H6′-cyclopentante-equatorial). APCI: calc. for C₂₃H₃₀BrFN₆O₂ [M+H]⁺: 520.2, found: 520.6/522.5. HPLC: t_{R}=10.499 min (Method A). UV-purity at 210 nm=97.0%.

### tert-butyl (((3αR,4R,6R,6αS)-6-(5-bromo-4-((4-methoxybenzyl)amino)-7H-pyrrolo[2,3-d]pyrimidin-7-yl)-2,2-dimethyltetrahydro-4H-cyclopenta[d][1,3]dioxol-4-yl)methyl)(3-((4-fluorophenethyl) amino)propyl)carbamate (318) & (1R,2S,3R,5R)-3-(4-amino-5-bromo-7H-pyrrolo[2,3-d]pyrimidin-7-yl)-5-(((3-((4-fluorophenethyl) amino)propyl) amino) methyl) cyclopentane-1,2-diol (319)

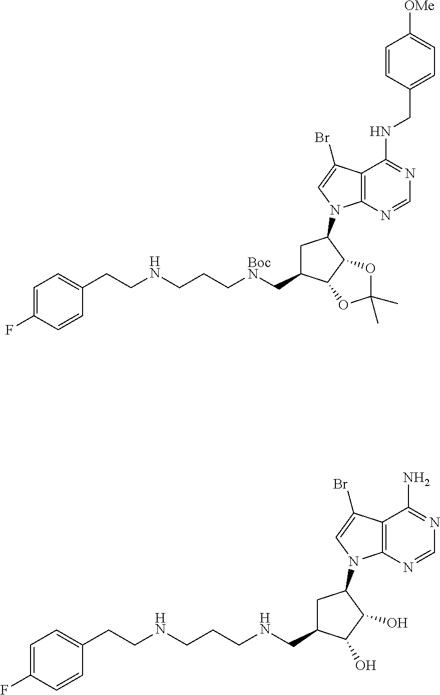 [SMILES: COc1ccc(CNc2ncnc3c2c(Br)cn3[C@@H]2C[C@H](CN(CCCNCCc3ccc(F)cc3)C(=O)OC(C)(C)C)[C@H]3OC(C)(C)O[C@H]32)cc1 ; Nc1ncnc2c1c(Br)cn2[C@@H]1C[C@H](CNCCCNCCc2ccc(F)cc2)[C@@H](O)[C@H]1O]

According to general procedure D. Reductive amination (318): yield: 36.7 mg, 0.05 mmol, 78%. C₃₉H₅₀BrFN₆O₅ (781.77 g/mol). APCI: calc. [M+H]⁺: 780.3, found: 780.5. According to general procedure O. Deprotection (319): C₂₃H₃₀BrFN₆O₂ (521.44 g/mol). The crude product was purified by preparative HPLC according to method D. yield: 19.24 mg, 0.04 mmol, 97% (2 TFA salt). 1H NMR (400 MHZ, DMSO-d₆) δ 8.85 (bs, 2H, NH₂+), 8.69 (bs, 2H, NH₂+), 8.30 (s, 1H, H2), 7.83 (s, 1H, H6), 7.37-7.27 (m, 2H, m′), 7.24-7.14 (m, 2H, o′), 4.95 (dt, J=10.4, 7.9 Hz, 1H, H1′), 4.18 (dd, J=7.6, 5.6 Hz, 1H, H2′), 3.88 (t, J=5.2 Hz, 1H, H3′), 3.22-3.13 (m, 2H, CH₂, H5′, H4″), 3.09-2.99 (m, 4H, CH₂, H1″, H3″), 2.96-2.88 (m, 2H, CH₂, H5″), 2.37-2.20 (m, 2H, H4′, H6-cyclopentane-axial), 1.98 (p, J=7.5 Hz, 2H, CH₂, H2″), 1.66-1.55 (m, 1H, H6, cyclopentane-equatorial). APCI: calc. for C₂₃H₃₀BrFN₆O₂ [M+H]⁺: 520.2, found: 520.6/522.5. HPLC: t_{R}=10.562 min (Method A). UV-purity at 210 nm=95.3%.

### tert-butyl (4-(benzyloxy)benzyl)(3-((((3αR,4R,6R,6αR)-6-(6-((tert-butoxycarbonyl)amino)-9H-purin-9-yl)-2,2-dimethyltetrahydrofuro[3,4-d][1,3]dioxol-4-yl)methyl) amino)propyl)carbamate (320) & (2R,3R,4S,5R)-2-(6-amino-9H-purin-9-yl)-5-(((3-((4-(benzyloxy)benzyl)amino)propyl)amino)methyl)tetrahydrofuran-3 4-diol (321)

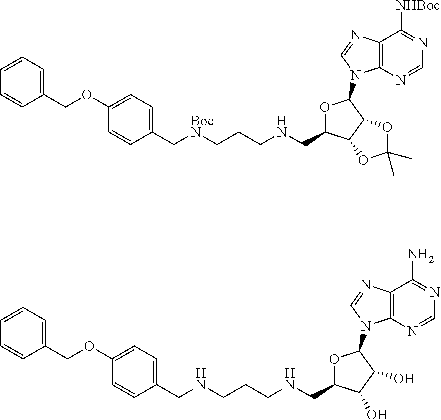 [SMILES: CC(C)(C)OC(=O)Nc1ncnc2c1ncn2[C@@H]1O[C@H](CNCCCN(Cc2ccc(OCc3ccccc3)cc2)C(=O)OC(C)(C)C)[C@H]2OC(C)(C)O[C@H]21 ; Nc1ncnc2c1ncn2[C@@H]1O[C@H](CNCCCNCc2ccc(OCc3ccccc3)cc2)[C@@H](O)[C@H]1O]

According to general procedure A. Reductive amination (320): yield: 37.80 mg, 6%. C₄₀H₅₃ N₇O₈ (759.91 g/mol). According to general procedure Q. Deprotection (321): C₂₇H₃ N₇O₄ (519.61 g/mol). yield: 11.60 mg, 30% (2 TFA salt). ¹H NMR (400 MHZ, DMSO-d₆) δ 8.87 (bs, 2H, NH₂+), 8.69 (bs, 2H, NH₂+), 8.45 (s, 1H, H2), 8.27 (s, 1H, H8), 7.83 (s, 2H, NH₂, N₆-amine), 7.47-7.43 (m, 2H, o″), 7.42-7.37 (m, 4H, m′, m″), 7.37-7.31 (m, 1H, p″), 7.11-7.06 (m, 2H, o′), 5.99 (d, J=5.3 Hz, 1H, H1′), 5.14 (s, 2H, CH₂, OBn), 4.71 (t, J=5.1 Hz, 1H, H2′), 4.27 (t, J=4.5 Hz, 1H, H3′), 4.24-4.18 (m, 1H, H4′), 4.06 (t, J=5.7 Hz, 2H, CH₂, H4″), 3.49-3.28 (m, 2H, CH₂, H5′), 3.08-2.98 (m, 2H, CH₂, H3″), 2.98-2.90 (m, 2H, CH₂, H1″), 2.00-1.89 (m, 2H, CH₂, H2″). HRMS: calc. for C₂₇H₃₃N₇O₄ [M+H]⁺: 520.3, found: 520.3. HPLC: t_{R}=8.323 min (Method A). UV-purity at 210 nm=95.1%.

### tert-butyl (3-((((3αR,4R,6R,6αR)-6-(6-((tert-butoxycarbonyl)amino)-9H-purin-9-yl)-2,2-dimethyltetrahydrofuro[3,4-d][1,3]dioxol-4-yl)methyl) amino)propyl)(naphthalen-2-ylmethyl)carbamate (322) & (2R,3R,45,5R)-2-(6-amino-9H-purin-9-yl)-5-(((3-((naphthalen-2-ylmethyl) amino)propyl) amino) methyl)tetrahydrofuran-3,4-diol (323)

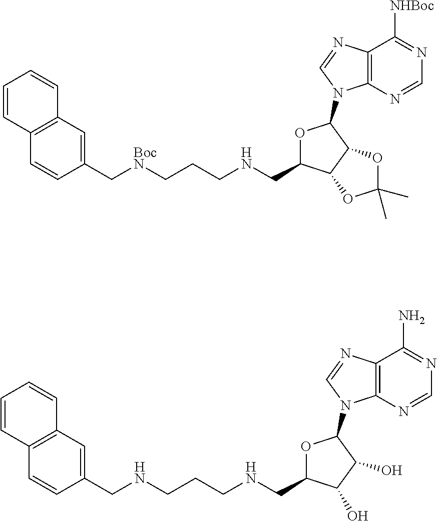 [SMILES: CC(C)(C)OC(=O)Nc1ncnc2c1ncn2[C@@H]1O[C@H](CNCCCN(Cc2ccc3ccccc3c2)C(=O)OC(C)(C)C)[C@H]2OC(C)(C)O[C@H]21 ; Nc1ncnc2c1ncn2[C@@H]1O[C@H](CNCCCNCc2ccc3ccccc3c2)[C@@H](O)[C@H]1O]

According to general procedure A. Reductive amination (322): yield: 90.30 mg, 16%. C₃₇H₄₉N₇O₇ (703.84 g/mol). According to general procedure Q. Deprotection (323): C₂₄H₃₉N₇O₃ (463.54 g/mol). yield: 19.00 mg, 22% (2 TFA salt). ¹H NMR (400 MHZ, DMSO-d₆) δ 9.13 (bs, 2H, NH₂+), 8.98-8.65 (m, 2H, NH₂+), 8.48 (s, 1H, H2), 8.30 (s, 1H, H8), 8.05-7.90 (m, 5H, naphtyl (H1′ “, H3”, H5′″), N₆-amine), 7.63-7.57 (m, 3H, naphtyl(H4′ “, H6”, H7′″), 5.99 (d, J=5.3 Hz, 1H, H1′), 4.70 (t, J=5.1 Hz, 1H, H2′), 4.33 (t, J=5.6 Hz, 2H, CH₂, H4″), 4.28-4.24 (m, 1H, H3′), 4.24-4.19 (m, 1H, H4′), 3.50-3.27 (m, 2H, CH₂, H5′), 3.11-2.97 (m, 4H, CH₂, H1″, H3″), 2.07-1.92 (m, 2H, CH₂, H2″). HRMS: calc. for C₂₄H₂₉N₇O₃ [M+H]⁺: 464.2, found: 464.2. HPLC: t_{R}=7.029 min (Method A). UV-purity at 210 nm=95.1%.

### (3αS,4S,6R,6αR)-6-(6-amino-9H-purin-9-yl)-2,2-dimethyl-N-(1-phenethylpiperidin-4-yl) tetrahydrofuro[3,4-d][1,3]dioxole-4-carboxamide (324) & (2S,3S,4R,5R)-5-(6-amino-9H-purin-9-yl)-3,4-dihydroxy-N-(1-phenethylpiperidin-4-yl) tetrahydrofuran-2-carboxamide (325)

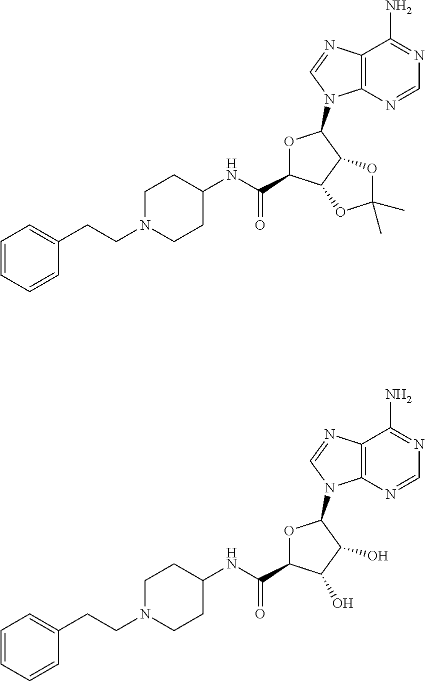 [SMILES: CC1(C)O[C@@H]2[C@H](O1)[C@@H](C(=O)NC1CCN(CCc3ccccc3)CC1)O[C@H]2n1cnc2c(N)ncnc21 ; Nc1ncnc2c1ncn2[C@@H]1O[C@H](C(=O)NC2CCN(CCc3ccccc3)CC2)[C@@H](O)[C@H]1O]

According to general procedure E. Peptidic coupling (324): yield: 66.30 mg, 41%. C₂₆H₃ N₇O₄ (507.60 g/mol). According to general procedure Q. Deprotection (325): C₂₃H₂₉N₇O₄ (467.53 g/mol). yield: 85 mg, quant. (1 TFA salt). ¹H NMR (400 MHZ, DMSO-d₆) δ 9.54 (s, 1H, NH₂⁺), 8.90 (d, J=8.1 Hz, 1H, NH, amide), 8.60 (s, 1H, H2), 8.49 (s, 1H, H8), 8.17 (s, 2H, N₆-amine), 7.45-7.37 (m, 2H, o′), 7.36-7.30 (m, 3H, m′, p′), 6.04 (d, J=7.4 Hz, 1H, H1′), 4.64 (dd, J=7.4, 4.5 Hz, 1H, H2′), 4.41 (d, J=1.7 Hz, 1H, H4′), 4.22 (dd, J=4.6, 1.7 Hz, 1H, H3′), 4.05-3.97 (m, 1H, H1″), 3.69 (t, J=11.2 Hz, 2H, H5″, CH₂, piperidine), 3.40-3.31 (m, 2H, CH₂NH⁺), 3.23-3.10 (m, 2H, H3″, CH₂, piperidine), 3.09-2.99 (m, 2H, CH₂Ph), 2.17-1.98 (m, 2H, H6″, CH₂, piperidine), 1.86-1.70 (m, 2H, H2″, CH₂, piperidine). HRMS: calc. for C₂₃H₂₉N₇O₄ [M+H]⁺: 468.2, found: 468.2. HPLC: t_{R}=7.029 min (Method A). UV-purity at 210 nm=95.1%.

### tert-butyl (3-((3αS,45,6R,6αR)-6-(6-amino-9H-purin-9-yl)-2,2-dimethyltetrahydrofuro[3,4-d [1,3]dioxole-4-carboxamido)propyl)(2-(naphthalen-2-yl)ethyl)carbamate (687) & (2S,3S,4R,5R)-5-(6-amino-9H-purin-9-yl)-3,4-dihydroxy-N-(3-((2-(naphthalen-2-yl)ethyl) amino)propyl) tetrahydrofuran-2-carboxamide (688)

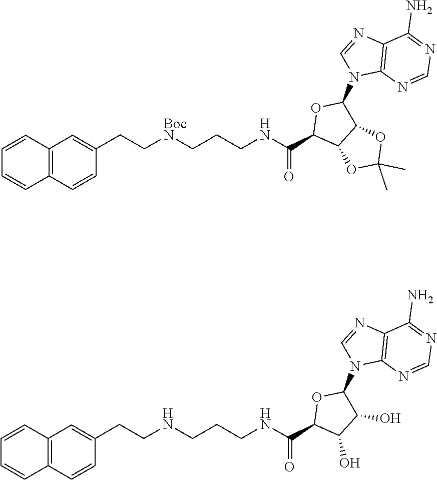 [SMILES: CC(C)(C)OC(=O)N(CCCNC(=O)[C@H]1O[C@@H](n2cnc3c(N)ncnc32)[C@@H]2OC(C)(C)O[C@@H]21)CCc1ccc2ccccc2c1 ; Nc1ncnc2c1ncn2[C@@H]1O[C@H](C(=O)NCCCNCCc2ccc3ccccc3c2)[C@@H](O)[C@H]1O]

According to general procedure E. Peptidic coupling (687): yield: 111.8 mg, 58%. C₃₃H₄]N₇₀₆ (631.73 g/mol). According to general procedure Q. Deprotection (688): C₂₅H₂₉N₇O₄ (491.55 g/mol). yield: 47.00 mg, 100% (1 TFA salt). ¹H NMR (400 MHZ, DMSO-d₆) δ 8.98 (t, J=5.9 Hz, 1H, NH, amide), 8.59 (s, 3H, H2, NH₂+), 8.40 (s, 1H, H8), 8.26 (bs, 2H, N₆-amine), 7.94-7.85 (m, 3H, naphtyl, H4′″, H5″, H8″), 7.80-7.76 (m, 1H, H1′″, naphtyl), 7.56-7.47 (m, 2H, H6′ “, H7′”, naphtyl), 7.44 (dd, J=8.4, 1.8 Hz, 1H, H3″ “, naphtyl), 6.02 (d, J=7.2 Hz, 1H, H1′), 4.61 (dd, J=7.2, 4.5 Hz, 1H, H2′), 4.38 (d, J=1.9 Hz, 1H, H4′), 4.21 (dd, J=4.5, 1.9 Hz, 1H, H3′), 3.34-3.22 (m, 4H, CH₂, H1”, H4″), 3.13-3.04 (m, 2H, CH₂, H5″), 3.04-2.95 (m, 2H, CH₂, H3″), 1.83 (d, J=6.7 Hz, 2H, CH₂, H2″). HRMS: calc. for C₂₅H₂₉N₇O₄ [M+H]⁺: 492.2, found: 492.2. HPLC: t_{R}=9.854 min (Method A). UV-purity at 210 nm=93.2%.

### (1R,2S,3R,5R)-3-(4-(methylamino)-7H-pyrrolo[2,3-d]pyrimidin-7-yl)-5-((2-(3-((3-phenoxyphenethyl) amino)propyl)-1H-imidazol-1-yl)methyl) cyclopentane-1,2-diol (326)

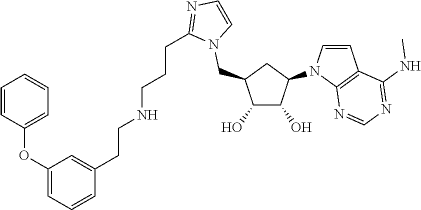 [SMILES: CNc1ncnc2c1ccn2[C@@H]1C[C@H](Cn2ccnc2CCCNCCc2cccc(Oc3ccccc3)c2)[C@@H](O)[C@H]1O]

To a solution of tert-butyl (7-((3αS,4R,6R,6αR)-2,2-dimethyl-6-((2-(3-((3-phenoxyphenethyl) amino)propyl)-1H-imidazol-1-yl)methyl)tetrahydro-4H cyclopenta[d][1,3]dioxol-4-yl)-7H-pyrrolo[2,3-a]pyrimidin-4-yl)(methyl)carbamate (10 mg, 0.01 mmol) in DCM (3 mL) was added TFA (3 mL). The solution was stirred at room temperature for 3 hrs. The mixture was concentrated to dryness and the residue was purified by prep-HPLC(TFA buffer) to give 326 (1.04 mg, yield: 12.5%) as a white solid. ¹H NMR (400 MHZ, CD₃OD): δ=8.21 (s, 1H), 7.51-7.46 (m, 2H), 7.36-7.32 (m, 4H), 7.13-6.83 (m, 7H), 5.01-4.95 (m, 1H), 4.44-4.26 (m, 3H), 4.07 (t, J=6.4 Hz, 1H), 3.31-3.27 (m, 2H), 3.24-3.11 (m, 7H), 3.00 (t, J=8 Hz, 2H), 2.54-2.48 (m, 1H), 2.39-2.32 (m, 1H), 2.22-2.18 (m, 2H), 1.90-1.81 (m, 1H). MS Calc.: 581.3; MS Found: 582.4 [M+H⁺].

### tert-butyl (7-((3αS,4R,6R,6αR)-6-(((3-((4-(benzyloxy)benzyl)(tert-butoxycarbonyl) amino)propyl) amino)methyl)-2,2-dimethyltetrahydro-4H-cyclopenta[d][1,3]dioxol-4-yl)-7H-pyrrolo[2,3-d]pyrimidin-4-yl)(methyl)carbamate (327) & (1S,2R,3R,5R)-3-(((3-((4-(benzyloxy)benzyl) amino)propyl) amino)methyl)-5-(4-(methylamino)-7H-pyrrolo[2,3-d]pyrimidin-7-yl) cyclopentane-1,2-diol (328)

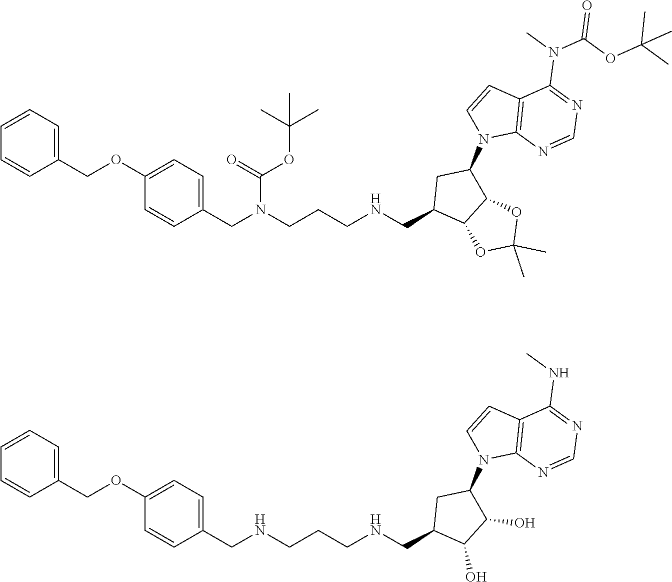 [SMILES: CN(C(=O)OC(C)(C)C)c1ncnc2c1ccn2[C@@H]1C[C@H](CNCCCN(Cc2ccc(OCc3ccccc3)cc2)C(=O)OC(C)(C)C)[C@H]2OC(C)(C)O[C@H]21 ; CNc1ncnc2c1ccn2[C@@H]1C[C@H](CNCCCNCc2ccc(OCc3ccccc3)cc2)[C@@H](O)[C@H]1O]

According to general procedure D. Reductive amination (327): yield: 54.00 mg, 26%. C₄₃H₅₈N₆O₇ (770.97 g/mol). According to general procedure Q. Deprotection (328): C₂₇H₃₄N₈O₂ (502.62 g/mol). yield: 5 mg, 10% (2 TFA salt). ¹H NMR (400 MHZ, DMSO-d₆) δ 8.93 (bs, 2H, NH₂*), 8.66 (bs, 2H, NH₂+), 8.29 (s, 1H, H2), 7.50 (bs, 1H, H8), 7.47-7.37 (m, 6H, o H, m′-H, m-H) 7.36-7.31 (m, 1H, p′-H) 7.11-7.07 (m, 2H, o-H), 6.80 (bs,1H, H7), 5.14 (s, 2H, Ph-CH₂—O—), 4.94-4.83 (m, 1H, H1′), 4.20-4.14 (m, 1H, H2′), 4.11-4.05 (m, 2H, —Ar—CH₂—NH—), 3.88 (t, J=5.4 Hz, 1H, H3′), 3.22-3.13 (m, 2H, H5′), 3.12-2.90 (m, 8H, H5′, H1″, H3″, —NH—CH₃) 2.30-2.16 (m, 2H, H4′, H6′A), 2.04-1.89 (m, 2H, H2″), 1.71-1.53 (m, 1H, H6′B). HRMS: calc. for C₃₀H₃₈N₆O₃ [M+H]⁺: 530.3 found: 530.1. HPLC: t_{R}=8.090 min. UV purity at 210 nm=99%.

### tert-butyl (7-((3αS,4R,6R,6αR)-6-(((3-((tert-butoxycarbonyl)(phenethyl) amino)propyl)(2,2,2-trifluoroethyl) amino)methyl)-2,2-dimethyltetrahydro-4H-cyclopenta[d][1,3]dioxol-4-yl)-7H-pyrrolo[2,3-d]pyrimidin-4-yl)(methyl)carbamate (329) & (1R,2S,3R,5R)-3-(4-(methylamino)-7H-pyrrolo[2,3-d]pyrimidin-7-yl)-5-(((3-(phenethylamino)propyl)(2,2,2-trifluoroethyl) amino) methyl) cyclopentane-1,2-diol (330)

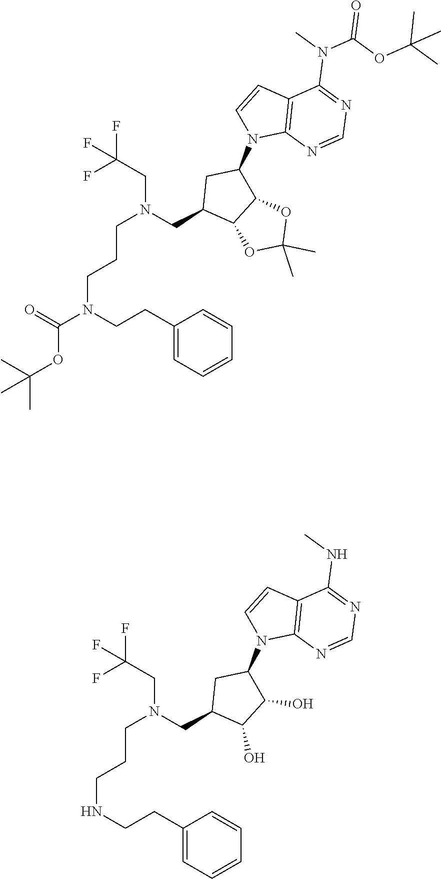 [SMILES: CN(C(=O)OC(C)(C)C)c1ncnc2c1ccn2[C@@H]1C[C@H](CN(CCCN(CCc2ccccc2)C(=O)OC(C)(C)C)CC(F)(F)F)[C@H]2OC(C)(C)O[C@H]21 ; CNc1ncnc2c1ccn2[C@@H]1C[C@H](CN(CCCNCCc2ccccc2)CC(F)(F)F)[C@@H](O)[C@H]1O]

According to general procedure D. Reductive amination (329): yield: 85.00 mg, 80%. C₃₉H₅₅F₃N₆O₆ (760.90 g/mol). APCI: calc. for C₃₉H₅₅F3N₆O₆ [M+H]⁺: 760.4, found: 660/560. According to general procedure Q. Deprotection (330): C₂₆H₃₅F3N₆O₂ (502.62 g/mol). yield: 84 mg, quantitative (1 TFA salt). ¹H NMR (400 MHZ, DMSO-d₆) δ 9.61 (bs, 1H, —NH—CH₃), 8.62 (bs, 2H, NH₂+), 8.36 (s, 1H, H2), 7.67-7.60 (m, 1H, H8), 7.37-7.31 (m, 2H, m-H), 7.29-7.22 (m, 3H, o-H, p-H), 6.96-6.87 (m, 1H, H7), 4.97 (q, J=9.2 Hz, 1H, H1′), 4.22 (dd, J=8.9, 5.4 Hz, 1H, H2′), 3.75 (dd, J=5.4, 3.1 Hz, 1H, H3′), 3.30 (q, J=10.0 Hz, 2H, —CH₂—CF₃), 3.18-3.13 (m, 2H, H4″), 3.12-3.05 (m, 3H, —NH—CH₃), 3.00-2.93 (m, 2H, H3″), 2.92-2.85 (m, 2H, H5″), 2.81 (dd, J=12.8, 7.2 Hz, 1H, H5′A), 2.72-2.65 (m, 2H, H1″), 2.62-2.56 (m, 1H, H5 B), 2.28-2.18 (m, 1H, H6′), 2.14 (td, J=8.3, 3.2 Hz, 1H, H4′), 1.77 (p, 2H, J=7.82 Hz, 2H, H2″), 1.51-1.41 (m, 1H, H6′). APCI calc. for C₂₆H₃₅F3N₆₀₂ [M+H]⁺: 520.3, found: 520.9. HPLC: t_{R}=13.08 min, UV purity at 210 nm=90%.

### (1R,2S,3R,5R)-3-(4-(methylamino)-7H-pyrrolo[2,3-d]pyrimidin-7-yl)-5-(((3-((4-phenoxybenzyl) amino)propyl) amino) methyl) cyclopentane-1,2-diol (331) & (1R,2S,3R,5R)-3-(4-(methylamino)-7H-pyrrolo[2,3-d]pyrimidin-7-yl)-5-(((3-((4-phenoxybenzyl) amino)propyl) amino) methyl) cyclopentane-1,2-diol (332)

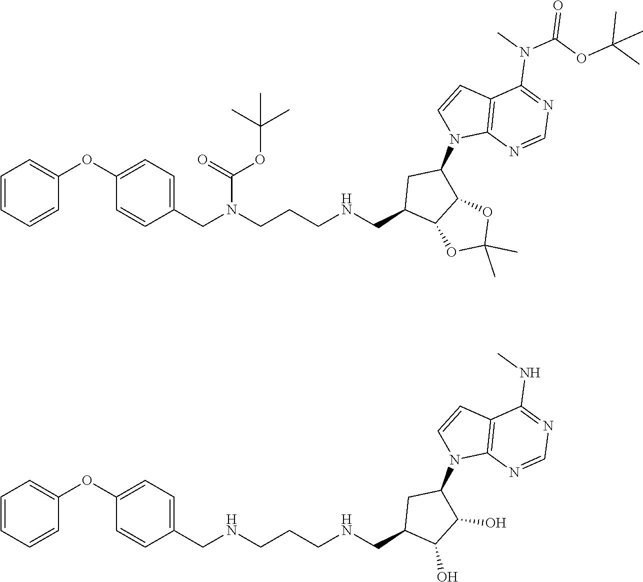 [SMILES: CN(C(=O)OC(C)(C)C)c1ncnc2c1ccn2[C@@H]1C[C@H](CNCCCN(Cc2ccc(Oc3ccccc3)cc2)C(=O)OC(C)(C)C)[C@H]2OC(C)(C)O[C@H]21 ; CNc1ncnc2c1ccn2[C@@H]1C[C@H](CNCCCNCc2ccc(Oc3ccccc3)cc2)[C@@H](O)[C@H]1O]

According to general procedure D. Reductive amination (331): yield: 11.00 mg, 7%. C₄₂H₅₆N₆O₇ (756.95 g/mol). APCI: calc. for C₄₂H₅₆N₆O₇ [M+H]⁺: 756.4, found: 756.9/656.9. According to general procedure Q. Deprotection (332): C₂₉H₃₆N₆O₃ (516.65 g/mol). yield: 4.20 mg, 42% (2 TFA salt). ¹H NMR (400 MHZ, DMSO-d₆) δ 9.32 (bs, 1H, —NH—CH₃), 9.02 (bs, 2H, NH₂+), 8.70 (bs, 2H, NH₂+), 8.33 (s, 1H, H2), 7.58 (bs, 1H, H8), 7.52-7.46 (m, 2H, m-H), 7.45-7.38 (m, 2H, m-H′), 7.21-7.16 (m, 1H, p-H′), 7.10-7.04 (m, 2H, o-H), 7.04-6.99 (m, 2H, o-H′), 6.87 (bs, 1H, H7), 4.92 (q, J=7.36 Hz, 1H, H1′), 4.21-4.08 (m, 3H, H2′), 3.88 (t, J=5.4 Hz, 1H, H3′), 3.24-3.13 (m, 1H, H4″), 3.12-2.90 (m, 8H, H5′, H1″, H3″, —NH—CH₃),2.33-2.21 (m, 1H, H4′), 2.35-2.28 (m, 1H, H6′A), 2.05-1.90 (m, 2H, H2″), 1.60 (q, J=10.2 Hz, 1H, H6′B). APCI calc. for C₂₉H₃₆N₆O₃ [M+H]⁺: 516.3, found: 516.7.). HPLC: t_{R}=12.997 min, UV purity at 210 nm=99%.

### tert-butyl (((3αR,4R,6R,6αS)-6-(5-bromo-4-((4-methoxybenzyl)amino)-7H-pyrrolo[2,3-d]pyrimidin-7-yl)-2,2-dimethyltetrahydro-4H-cyclopenta[d][1,3]dioxol-4-yl)methyl)(3-((2,4-difluorophenethyl) amino)propyl)carbamate (333) & (1R,2S,3R,5R)-3-(4-amino-5-bromo-7H-pyrrolo[2,3-d]pyrimidin-7-yl)-5-(((3-((2,4-difluorophenethyl) amino)propyl) amino) methyl) cyclopentane-1,2-diol (334)

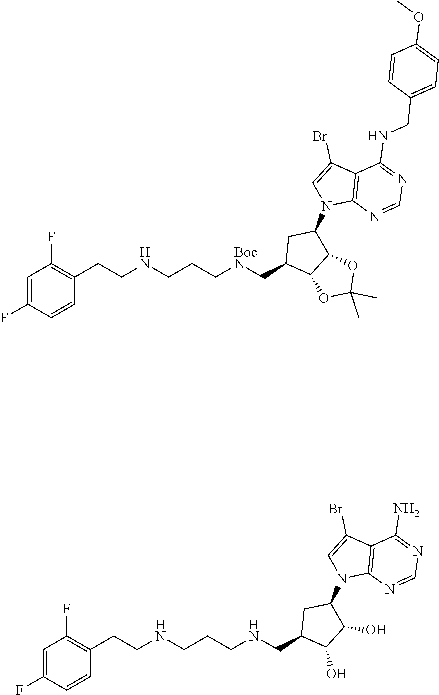 [SMILES: COc1ccc(CNc2ncnc3c2c(Br)cn3[C@@H]2C[C@H](CN(CCCNCCc3ccc(F)cc3F)C(=O)OC(C)(C)C)[C@H]3OC(C)(C)O[C@H]32)cc1 ; Nc1ncnc2c1c(Br)cn2[C@@H]1C[C@H](CNCCCNCCc2ccc(F)cc2F)[C@@H](O)[C@H]1O]

According to general procedure D. Reductive amination (333): yield: 19.00 mg, 52%. C₃₉H₄₉BrF₂N₆O₃ (799.76 g/mol). APCI: calc. [M+H]⁺: 798.3, found: 800.5/798.5 . . . . According to general procedure O. Deprotection (334): C₂₃H₂₉BrF₂N₆O₂ (539.43 g/mol). The crude product was purified by preparative HPLC according to method D. yield: 12.00 mg, 80% (2 TFA salt). ¹H NMR (400 MHZ, DMSO-d₆) δ 8.80 (bs, 2H, NH₂+), 8.61 (bs, 2H, NH₂+), 8.23 (s, 1H, H2), 7.79 (s, 1H, H6), 7.42-7.35 (m, 1H, m-H,), 7.30-7.22 (m, 1H, m-H), 7.12-7.05 (td, J=8.7, 2.7 Hz, 1H, o-H), 4.94 (dt, J=9.6 Hz, 1H, H1′), 4.16 (dd, J=7.6, 5.6 Hz, 1H, H2′), 3.86 (t, J=5.2 Hz, 1H, H3′), 3.20-3.08 (m, 2H, H4″), 3.07-2.96 (m, 6H, H5′, H1″, H3″), 2.95-2.88 (m, 2H, H5″), 2.34-2.16 (m, 2H, H4′, H6-cyclopentane-axial), 2.03-1.88 (m, 2H, CH₂, H2″), 1.62-1.52 (m, 1H, H6, cyclopentane-equatorial). APCI: calc. For C₂₃H₂₉BrF₂N₆₀₂ [M+H]*: 538,2, found: 538.5/540.5. HPLC: t_{R}=9.840 min. UV-purity at 210 nm=97%.

### tert-butyl (((3αR,4R,6R,6αS)-6-(5-bromo-4-((4-methoxybenzyl)amino)-7H-pyrrolo[2,3-d]pyrimidin-7-yl)-2,2-dimethyltetrahydro-4H-cyclopenta[d][1,3]dioxol-4-yl)methyl)(3-((4-(trifluoromethyl)phenethyl) amino)propyl)carbamate (335) & (1R,2S,3R,5R)-3-(4-amino-5-bromo-7H-pyrrolo[2,3-d]pyrimidin-7-yl)-5-(((3-((4-(trifluoromethyl)phenethyl) amino)propyl) amino) methyl) cyclopentane-1,2-diol (336)

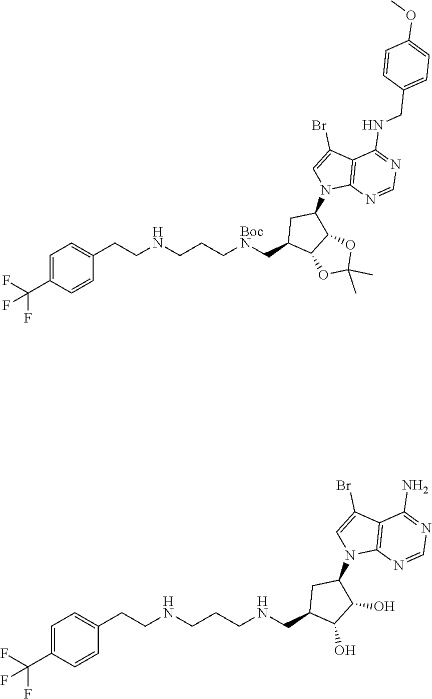 [SMILES: COc1ccc(CNc2ncnc3c2c(Br)cn3[C@@H]2C[C@H](CN(CCCNCCc3ccc(C(F)(F)F)cc3)C(=O)OC(C)(C)C)[C@H]3OC(C)(C)O[C@H]32)cc1 ; Nc1ncnc2c1c(Br)cn2[C@@H]1C[C@H](CNCCCNCCc2ccc(C(F)(F)F)cc2)[C@@H](O)[C@H]1O]

According to general procedure D. Reductive amination (335): yield: 17.00 mg, 58%. C₄₀H₅₀BrF₃N₆O₅ (831.73 g/mol). APCI: calc. [M+H]⁺: 830.3, found: 832/830. According to general procedure O. Deprotection (336): C₂₄H₃₀BrF₃N₆₀₂ (571.44 g/mol). The crude product was purified by preparative HPLC according to method D. yield: 10.00 mg, 77% (2 TFA salt). ¹H NMR (400 MHZ, DMSO-d₆) δ 8.79 (bs, 2H, NH₂+), 8.59 (bs, 2H, NH₂+), 8.22 (s, 1H, H2), 7.75-7.70 (m, 3H, H6, m-H), 7.53-7.48 (d, J=8.1 Hz, 2H, o-H), 4.90 (dt, J=10.2 Hz, 7.8 Hz, 1H, H1′), 4.16 (dd, J=7.6, 5.6 Hz, 1H, H2′), 3.86 (t, J=5.2 Hz, 1H, H3′), 3.26-3.16 (m, 2H, H4″, H5′), 3.08-2.95 (m, 7H, CH₂, H5′, H1″, H3″, H5″), 2.32-2.16 (m, 2H, H4′, H6′-axial), 2.00-1.88 (m, 2H, CH₂, H2″), 1.63-1.52 (m, 1H, H6′-equatorial). APCI: calc. For C₂₄H₃₀BrF₃N₆₀₂ [M+H]⁺: 570.2, found: 570.4/572.4. HPLC: (R=11.953. UV-purity at 210 nm=96%.

### tert-butyl (3-((((3αR,4R,6R,6as)-6-(5-bromo-4-((4-methoxybenzyl)amino)-7H-pyrrolo[2,3-d]pyrimidin-7-yl)-2,2-dimethyltetrahydro-4H-cyclopenta[d][1,3]dioxol-4-yl)methyl) amino)propyl)(2-fluorophenethyl)carbamate (337) & (1R,2S,3R,5R)-3-(4-amino-5-bromo-7H-pyrrolo[2,3-d]pyrimidin-7-yl)-5-(((3-((2-fluorophenethyl) amino)propyl) amino) methyl) cyclopentane-1,2-diol (338)

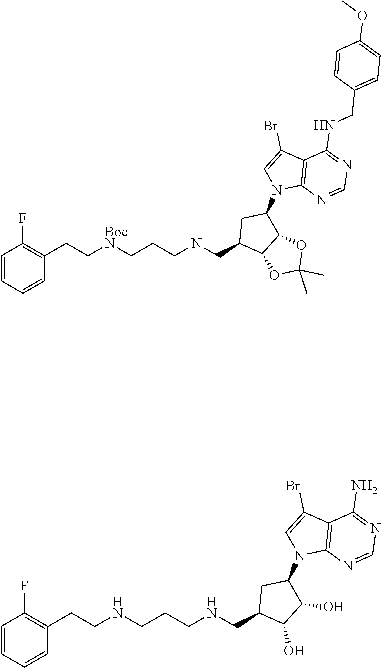

According to general procedure D. Reductive amination (337): yield: 45.00 mg, 64%. C₃₉H₅₀BrFN₆O₅ (781.7 g/mol). APCI: calc. [M+H]⁺: 781.8, found: 780.4/782.3/784.4. According to general procedure O. Deprotection (338): C₂₃H₃₀BrFN₆O₂ (521.44 g/mol). The crude product was purified by preparative HPLC according to method D. yield: 35.00 mg, 81% (2 TFA salt). 1H-NMR: (400 MHZ, DMSO-d₆) δ=8.87 (bs, 2H), 8.63 (bs, 2H), 8.25 (s, 1H), 7.77 (s, 1H), 7.39-7.32 (m, 2H), 7.25-7.17 (m, 2H), 4.96-4.89 (m, 1H), 4.20-4.14 (m, 1H), 3.89-3.84 (m, 1H), 3.25-3.11 (m, 3H), 3.10-2.91 (m, 8H), 2.35-2.32 (m, 1H), 2.31-2.18 (m, 2H), 2.01-1.93 (m, 2H), 1.63-1.55 (m, 1H) ppm. APCI: calc. For C₂₃H₃₀BrFN₆₀₂ [M+H]⁺: 521.4, found: 520.5/522.4. HPLC: {R=10.02 min. UV-purity at 210 nm=99%. tert-butyl (3-((((3αR,4R,6R,6as)-6-(4-((4-methoxybenzyl)amino)-5-phenyl-7H-pyrrolo[2,3-d]pyrimidin-7-yl)-2,2-dimethyltetrahydro-4//-cyclopenta[d][1,3]dioxol-4-yl)methyl) amino)propyl)(phenethyl)carbamate (339) & (1R,2S,3R,5R)-3-(4-amino-5-phenyl-7H-pyrrolo[2,3-d]pyrimidin-7-yl)-5-(((3-((4-fluorophenethyl) amino)propyl) amino) methyl) cyclopentane-1,2-diol (340)

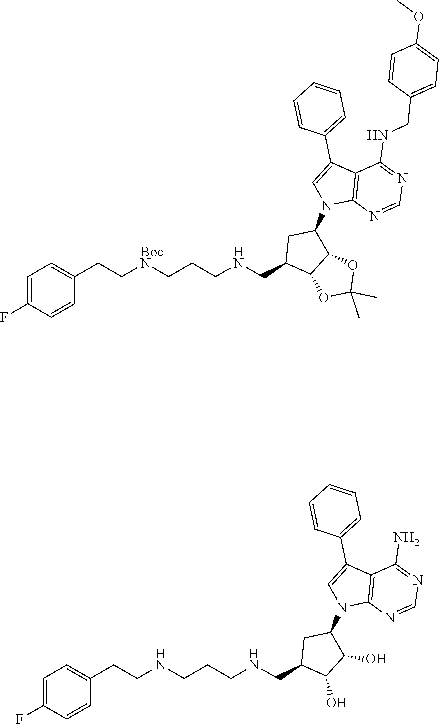 [SMILES: COc1ccc(CNc2ncnc3c2c(-c2ccccc2)cn3[C@@H]2C[C@H](CNCCCN(CCc3ccc(F)cc3)C(=O)OC(C)(C)C)[C@H]3OC(C)(C)O[C@H]32)cc1 ; Nc1ncnc2c1c(-c1ccccc1)cn2[C@@H]1C[C@H](CNCCCNCCc2ccc(F)cc2)[C@@H](O)[C@H]1O]

According to general procedure D. Reductive amination (339): yield: 160.00 mg, 37%. C₄₅H₅₅FN₆O₅ (778.97 g/mol). APCI: calc. [M+H]⁺: 778.9, found: 778.7/779.7. According to general procedure O. Deprotection (340): C₂₉H₃₅FN₆O₂ (518.64 g/mol). The crude product was purified by preparative HPLC according to method D. yield: 51.00 mg, 33% (2 TFA salt). 1H-NMR: (400 MHZ, DMSO-d₆) δ=8.88 (bs, 2H), 8.75 (bs, 2H), 8.48 (s, 1H), 7.84 (s, 1H), 7.57-7.47 (m, 4H), 7.46-7.40 (m, 1H), 7.34-7.27 (m, 2H), 7.22-7.14 (m, 2H), 5.10-5.00 (m, 1H), 4.28-4.22 (m, 1H), 3.94-3.88 (m, 1H), 3.25-3.11 (m, 3H), 3.10-2.97 (m, 5H), 2.95-2.87 (m, 2H), 2.40-2.35 (m, 1H), 2.34-2.24 (m, 1H), 2.04-1.91 (m, 2H), 1.75-1.60 (m, 1H) ppm. APCI: calc. For C₂₉H₃₅FN₆O₂ [M+H]⁺: 518.6, found: 518.9/519.9. HPLC: ¿ R=11.76 min. UV-purity at 210 nm=99%.

### tert-butyl (3-((((3αR,4R,6R,6as)-6-(5-iodo-4-((4-methoxybenzyl)amino)-7H-pyrrolo[2,3-d]pyrimidin-7-yl)-2,2-dimethyltetrahydro-4H-cyclopenta[d][1,3]dioxol-4-yl)methyl)-amino)propyl)(phenethyl)carbamate (341) & (1R,2S,3R,5R)-3-(4-amino-5-iodo-7H-pyrrolo[2,3-d]pyrimidin-7-yl)-5-(((3 (phenethylamino)-propyl) amino) methyl) cyclopentane-1,2-diol (342)

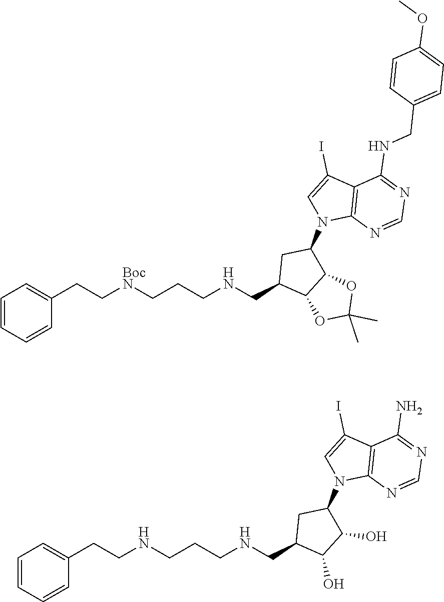 [SMILES: COc1ccc(CNc2ncnc3c2c(I)cn3[C@@H]2C[C@H](CNCCCN(CCc3ccccc3)C(=O)OC(C)(C)C)[C@H]3OC(C)(C)O[C@H]32)cc1 ; Nc1ncnc2c1c(I)cn2[C@@H]1C[C@H](CNCCCNCCc2ccccc2)[C@@H](O)[C@H]1O]

According to general procedure D. Reductive amination (342): yield: 83.00 mg, 51%. C₃₉H₅₁IN₆O₅ (810.78 g/mol). APCI: calc. [M+H]⁺: 810.8, found: 810.4. According to general procedure O. Deprotection (342): C₂₃H₃₁N₆O₂ (550.45 g/mol). The crude product was purified by preparative HPLC according to method D. yield: 40.00 mg, 53% (2 TFA salt). 1H-NMR: (400 MHZ, DMSO-d₆) δ=8.82 (s, 2H), 8.64 (s, 2H), 8.30 (s, 1H), 7.84 (s, 1H), 7.39-7.32 (m, 2H), 7.30-7.24 (m, 3H), 4.97-4.88 (m, 1H), 4.21-4.14 (m, 1H), 3.90-3.84 (m, 1H), 323-3.11 (m, 3H), 3.09-2.98 (m, 5H), 2.95-2.88 (m, 2H), 2.36-2.32 (m, 1H), 2.31-2.15 (m, 3H), 2.02-1.91 (m, 2H), 1.65-1.55 (m, 1H) ppm. APCI: calc. For C₂₃H₃₁ IN₆O₂ [M+H]⁺: 550.5, found: 550.5. HPLC: {R=10.25 min. UV-purity at 210 nm=99%.

### tert-butyl (3-((((3αR,4R,6R,6as)-6-(5-bromo-4-((4-methoxybenzyl)amino)-7H-pyrrolo[2,3-d]pyrimidin-7-yl)-2,2-dimethyltetrahydro-4H-cyclopenta[d][1,3]dioxol-4-yl)methyl) amino)propyl)(4-fluorobenzyl)carbamate (343) & (1R,2s,3R,5R)-3-(4-amino-5-bromo-7H-pyrrolo[2,3-d]pyrimidin-7-yl)-5-(((3-((4-fluorobenzyl) amino)propyl) amino) methyl) cyclopentane-1,2-diol (344)

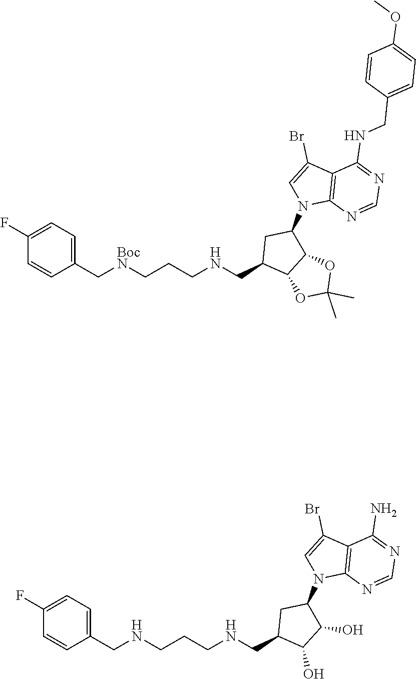 [SMILES: COc1ccc(CNc2ncnc3c2c(Br)cn3[C@@H]2C[C@H](CNCCCN(Cc3ccc(F)cc3)C(=O)OC(C)(C)C)[C@H]3OC(C)(C)O[C@H]32)cc1 ; Nc1ncnc2c1c(Br)cn2[C@@H]1C[C@H](CNCCCNCc2ccc(F)cc2)[C@@H](O)[C@H]1O]

According to general procedure D. Reductive amination (343): yield: 99.00 mg, 32%. C₃H 48BrFN₆₀₅ (767.74 g/mol). APCI: calc. [M+H]⁺: 767.7, found: 766.8/768.8/769.6/770.7. According to general procedure O. Deprotection (344): C₂₂H₂₈BrFN₆O₂ (507.41 g/mol). The crude product was purified by preparative HPLC according to method D. yield: 63.00 mg, 66% (2 TFA salt). 1H-NMR: (400 MHZ, DMSO-d₆) δ=8.99 (bs, 2H), 8.59 (bs, 2H), 8.27 (s, 1H), 7.80 (s, 1H), 7.57-7.52 (m, 2H), 7.39-7.27 (m, 2H), 4.98-4.87 (m, 2H), 4.20-4.13 (m, 4H), 3.89-3.84 (m, 1H), 3.22-3.10 (m, 1H), 3.08-2.92 (m, 5H), 2.35-2.32 (m, 1H), 2.31-2.18 (m, 1H), 2.02-1.93 (m, 2H), 1.62-1.54 (m, 1H) ppm. APCI: calc. For C₂₂H₂₈BrFN₆O₂ [M+H]⁺: 507.4, found: 506.9/508.9.4. HPLC: {R=9.32 min. UV-purity at 210 nm=98%.

### tert-butyl 4-(((1S,2R,3S,4R)-2,3-dihydroxy-4-(4-(methylamino)-7H-pyrrolo[2,3-a]pyrimidin-7-yl) cyclopentane-1-carboxamido) methyl) piperidine-1-carboxylate (345) & (1S,2R,3S,4R)-2,3-dihydroxy-4-(4-(methylamino)-7H-pyrrolo[2,3-d]pyrimidin-7-yl)-N-(piperidin-4-ylmethyl) cyclopentane-1-carboxamide (346)

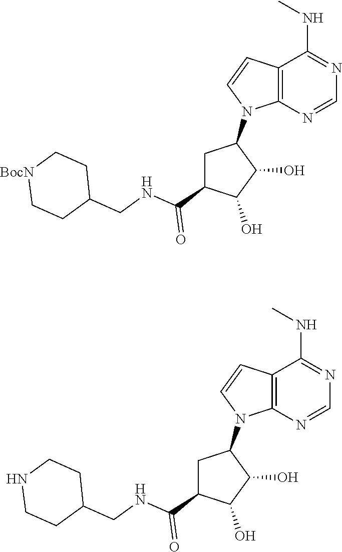 [SMILES: CNc1ncnc2c1ccn2[C@@H]1C[C@H](C(=O)NCC2CCN(C(=O)OC(C)(C)C)CC2)[C@@H](O)[C@H]1O ; CNc1ncnc2c1ccn2[C@@H]1C[C@H](C(=O)NCC2CCNCC2)[C@@H](O)[C@H]1O]

According to general procedure F. Peptidic coupling (345): yield: 50 mg, 60%. C₂₄H₃₆N₆O₅ (488.59 g/mol). MS calc. [M+H]⁺: 488.3, found: 489.5. According to general procedure P. Deprotection (346): yield: 36.50 mg, 95% (1 TFA salt). ¹H NMR (400 MHZ, CD₃OD): δ=8.27 (s, 1H), 7.71 (d, J=2.8 Hz, 1H), 6.93 (s, 1H), 5.20-5.17 (m, 1H), 4.40-4.32 (m, 1H), 4.28-4.25 (m, 1H), 3.40-3.15 (m, 8H), 2.97-2.91 (m, 2H), 2.77-2.71 (m, 1H), 2.58-2.54 (m, 1H), 2.18-1.77 (m, 5H). Mass calc. for C₁₉H₂₈N₆O₃. MS calc.: 388.2; MS Found: 389.3 [M+H]⁺. Purified by method C. UV-purity >93%.

### tert-butyl 3-(((1S,2R,3S,4R)-2,3-dihydroxy-4-(4-(methylamino)-7H-pyrrolo[2,3-d]pyrimidin-7-yl) cyclopentane-1-carboxamido) methyl) piperidine-1-carboxylate (347) & (1S,2R,3S,4R)-2,3-dihydroxy-4-(4-(methylamino)-7H-pyrrolo[2,3-d]pyrimidin-7-yl)-N-(piperidin-3-ylmethyl) cyclopentane-1-carboxamide (348)

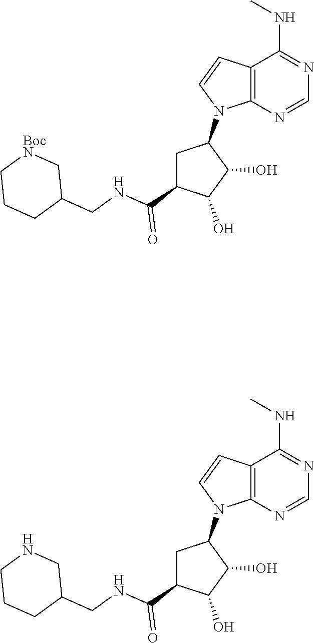 [SMILES: CNc1ncnc2c1ccn2[C@@H]1C[C@H](C(=O)NCC2CCCN(C(=O)OC(C)(C)C)C2)[C@@H](O)[C@H]1O ; CNc1ncnc2c1ccn2[C@@H]1C[C@H](C(=O)NCC2CCCNC2)[C@@H](O)[C@H]1O]

According to general procedure F. Peptidic coupling (347): yield: 50 mg, 60%. C₂₄H₃₆N₆O₅ (488.59 g/mol). MS calc. [M+H]⁺: 488.3, found: 489.3. According to general procedure P. Deprotection (348): yield: 36.00 mg, 95% (1 TFA salt). ¹H NMR (400 MHZ, DMSO-d₆): δ=9.98 (brs, 1H), 9.07 (brs, 1H), 8.86-8.83 (m, 1H), 8.36 (s, 1H), 8.29 (t, J=7.2 Hz, 1H), 7.73 (d, J=3.6 Hz, 1H), 7.07 (d, J=4.4 Hz, 1H), 5.02 (q,)=11.2 Hz, 1H), 4.24-4.19 (m, 1H), 4.08-4.05 (m, 1H), 3.06-2.58 (m, 8H), 2.58-2.53 (m, 2H) 2.00-1.50 (m, 6H), 1.32-1.05 (m, 1H). Mass calc. for C₁₉H₂₈N₆O₃. MS Calc.: 388.2; MS Found: 389.2 [M+H]⁺. Purified by method C. UV-purity >95%.

### tert-butyl 2-(((1S,2R,3S,4R)-2,3-dihydroxy-4-(4-(methylamino)-7H-pyrrolo[2,3-a]pyrimidin-7-yl) cyclopentane-1-carboxamido) methyl) piperidine-1-carboxylate (349) & (1S,2R,3S,4R)-2,3-dihydroxy-4-(4-(methylamino)-7H-pyrrolo[2,3-d]pyrimidin-7-yl)-N-(piperidin-2-ylmethyl) cyclopentane-1-carboxamide (350)

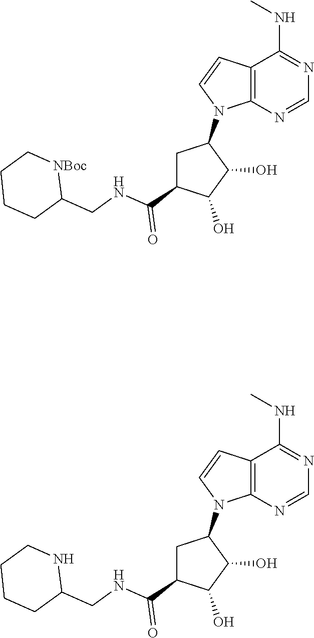 [SMILES: CNc1ncnc2c1ccn2[C@@H]1C[C@H](C(=O)NCC2CCCCN2C(=O)OC(C)(C)C)[C@@H](O)[C@H]1O ; CNc1ncnc2c1ccn2[C@@H]1C[C@H](C(=O)NCC2CCCCN2)[C@@H](O)[C@H]1O]

According to general procedure F. Peptidic coupling (349): yield: 50 mg, 60%. C₂₄H₃₆N₆O₅ (488.59 g/mol). MS calc. [M+H]⁺: 488.3, found: 489.3. According to general procedure P.

Deprotection (350): yield: 30.00 mg, 78% (1 TFA salt). ¹H NMR (400 MHZ, CD₃OD): δ=8.16 (s, 1H), 7.60 (d, J=3.2 Hz, 1H), 6.81 (s, 1H), 5.07 (q, J=7.2 Hz, 1H), 4.28-4.17 (m, 2H), 3.40-3.32 (m, 3H), 3.29-3.01 (m, 4H), 2.92-2.83 (m, 2H), 2.50-2.43 (m, 1H), 2.11-2.08 (m, 1H), 1.88-1.77 (m, 3H), 1.63-1.50 (m, 3H). Mass calc. for C₁₉H₂₈N₆O₃. MS Calc.: 388.2; MS Found: 389.2 [M+H]⁺. Purified by method C. UV-purity >99%.

### (1S,2R,3S,4R)-2,3-dihydroxy-N-methyl-4-(4-(methylamino)-7H-pyrrolo[2,3-a]pyrimidin-7-yl)-N-(3-((3-phenoxyphenethyl) amino)propyl) cyclopentane-1-carboxamide (351)

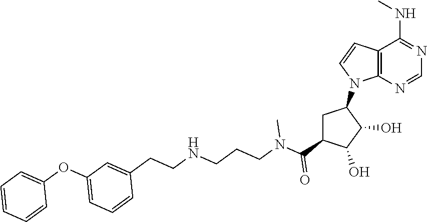 [SMILES: CNc1ncnc2c1ccn2[C@@H]1C[C@H](C(=O)N(C)CCCNCCc2cccc(Oc3ccccc3)c2)[C@@H](O)[C@H]1O]

According to general procedure F. Peptidic coupling (351): yield: 15 mg, 28% (HOAc salt). 1H NMR (400 MHZ, CD₃OD): δ=8.04 (s, 1H), 7.26-7.19 (m, 4H), 7.01-6.99 (m, 1H), 6.94-6.92 (m, 1H), 6.89-6.84 (m, 3H), 6.78-6.75 (m, 1H), 6.47-6.46 (m, 1H), 4.96-4.92 (m, 1H), 4.19-4.16 (m, 2H), 3.46-3.43 (m, 2H), 3.29-3.28 (m, 1H), 3.09-3.06 (m, 4H), 2.96-2.95 (m, 3H), 2.88-2.80 (m, 5H), 2.44 (m, 1H), 2.01-1.97 (m, 1H), 1.86-1.82 (m, 2H). Mass calc. for C₁₉H₂₈N₆O₃ (388.47 g/mol). Mass calc. for C₃₁H₃₈ N₆O₄. MS calc. [M+H]⁺: 558.3, found: 559.3 [M+H]⁺. Purified by method C(NH₄OAc buffer). UV-purity >91%.

### (1S,2R,3S,4R)—N-(3-((4-(4-chlorophenoxy)-3-fluorobenzyl) amino)propyl)-2,3-dihydroxy-4-(4-(methylamino)-7H-pyrrolo[2.3-d]pyrimidin-7-yl)cyclopentane-1-carboxamide (352)

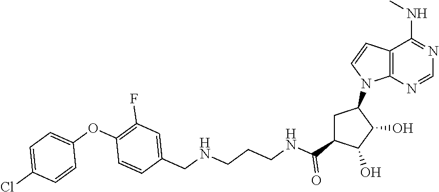 [SMILES: CNc1ncnc2c1ccn2[C@@H]1C[C@H](C(=O)NCCCNCc2ccc(Oc3ccc(Cl)cc3)c(F)c2)[C@@H](O)[C@H]1O]

According to general procedure F. Peptidic coupling (352): yield: 20 mg, 25% (free base). 1H NMR (400 MHZ, CD₃OD): δ=8.17 (s, 1H), 7.37-7.31 (m, 4H), 7.20 (d, J=8.0 Hz, 1H), 7.08 (t, J=8.4 Hz, 1H), 6.92 (d, J=8.4 Hz, 2H), 6.60 (d, J=3.2 Hz, 1H), 5.04 (dd, J=16.0, 6.8 Hz, 1H), 4.34-4.25 (m, 2H), 3.82 (s, 2H), 3.31-3.30 (m, 2H), 3.08 (s, 3H), 2.91-2.86 (m, 1H), 2.72-2.69 (m, 2H), 2.53-2.50 (m, 1H), 2.12-2.06 (m, 1H), 1.82-1.79 (m, 2H). Mass calc. for C₂₉H₃₂ClFN₆O₄. MS calc. [M+H]⁺: 582.2, found: 583.2 [M+H]⁺. Purified by method C(NH₄HCO₃ buffer). UV-purity >94%.

### (1S,2R,3S,4R)-2,3-dihydroxy-4-(4-(methylamino)-7H-pyrrolo[2,3-d]pyrimidin-7-yl)-N-(3-((3-phenoxybenzyl) amino)propyl) cyclopentane-1-carboxamide (353)

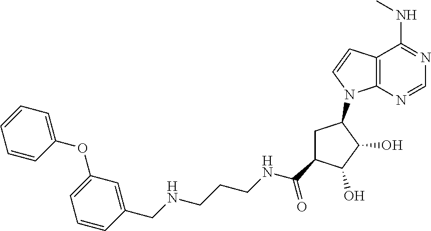 [SMILES: CNc1ncnc2c1ccn2[C@@H]1C[C@H](C(=O)NCCCNCc2cccc(Oc3ccccc3)c2)[C@@H](O)[C@H]1O]

According to general procedure F. Peptidic coupling (353): yield: 20 mg, 22.2% (TFA salt). 1H NMR (400 MHZ, DMSO-d₆): δ=8.76 (brs, 2H), 8.30 (s, 1H), 8.20 (t, J=6.0 Hz, 1H), 8.19 (s, 1H), 7.59 (brs, 1H), 7.50-7.37 (m, 3H), 7.25 (d, J=7.6 Hz, 1H), 7.23-7.14 (m, 2H), 7.05-7.00 (m, 3H), 6.84 (brs, 1H), 5.02-4.92 (m, 3H), 4.19-4.13 (m, 3H), 4.04 (t, J=4.8 Hz, 1H), 3.29-3.26 (m, 2H), 3.17 (s, 3H), 2.91-2.89 (m, 2H), 2.76-2.70 (m, 1H), 2.37-2.33 (m, 1H), 1.95-1.92 (m, 1H), 1.79-1.75 (m, 2H). Mass calc. for C₂₉H₃₄N₆O₄. MS calc. [M+H]⁺: 530.3, found: 531.3 [M+H]⁺. Purified by method C (TFA buffer). UV-purity >85%.

### (1S,2R,3S,4R)-2,3-dihydroxy-4-(4-(methylamino)-7H-pyrrolo[2,3-d]pyrimidin-7-yl)-N-(3-((3-phenoxyphenethyl) amino)propyl) cyclopentane-1-carboxamide (354)

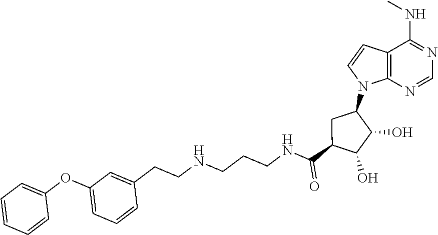 [SMILES: CNc1ncnc2c1ccn2[C@@H]1C[C@H](C(=O)NCCCNCCc2cccc(Oc3ccccc3)c2)[C@@H](O)[C@H]1O]

According to general procedure F. Peptidic coupling (354): yield: 15 mg, 27.5% (HOAc salt). 1H NMR (400 MHZ, CD₃OD): δ=8.04 (s, 1H), 7.25-7.19 (m, 4H), 7.02-7.00 (m, 1H), 6.94-6.92 (m, 1H), 6.88-6.83 (m, 3H), 6.78-6.76 (m, 1H), 6.48-6.47 (m, 1H), 4.93-4.87 (m, 1H), 4.20-4.14 (m, 2H), 3.27-3.24 (m, 2H), 3.07-3.03 (m, 2H), 2.96 (s, 3H), 2.86-2.83 (m, 4H), 2.79-2.75 (m, 1H), 2.45-2.38 (m, 1H), 2.02-1.97 (m, 1H), 1.78-1.75 (m, 2H). Mass calc. for C₃₀H₃₆N₆O₄. MS calc. [M+H]⁺: 544.2, found: 545.3 [M+H]⁺. Purified by method C(NH₄OAc buffer). UV-purity >96%.

### (1S,2R,3S,4R)—N-(3-((4-(4-chlorophenoxy)-3-fluorophenethyl) amino)propyl)-2,3-dihydroxy-4-(4-(methylamino)-7H-pyrrolo[2,3-a]pyrimidin-7-yl) cyclopentane-1-carboxamide (355)

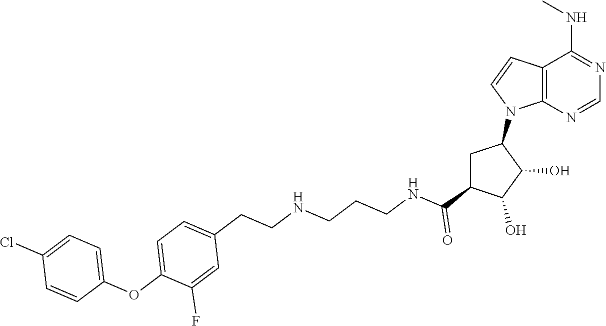 [SMILES: CNc1ncnc2c1ccn2[C@@H]1C[C@H](C(=O)NCCCNCCc2ccc(Oc3ccc(Cl)cc3)c(F)c2)[C@@H](O)[C@H]1O]

According to general procedure F. Peptidic coupling (355): yield: 12 mg, 15% (TFA salt). ¹H NMR (400 MHZ, CD₃OD): δ=8.26 (s, 1H), 7.64 (s, 1H), 7.33-7.26 (m, 3H), 7.18-7.12 (m, 2H), 6.94-6.88 (m, 3H), 5.15 (dd, J=16.0, 6.8 Hz 1H), 4.38-4.27 (m, 2H), 3.43-3.30 (m, 3H), 3.21 (s, 3H), 3.16-3.08 (m, 5H), 2.95-2.90 (m, 1H), 2.57-2.54 (m, 1H), 2.19-2.13 (m, 1H), 1.98-1.94 (m, 2H). MS calc. for C₃₀H₃₄ClFN₆O₄.MS calc. [M+H]⁺: 596.2, found: 597.3 [M+H]⁺. Purified by method C(TFA buffer). UV-purity >98%.

### (1S,2R,3S,4R)-2,3-dihydroxy-4-(4-(methylamino)-7H-pyrrolo[2,3-d]pyrimidin-7-yl)-N-(2-((3-phenoxyphenethyl) amino) ethyl) cyclopentane-1-carboxamide (356)

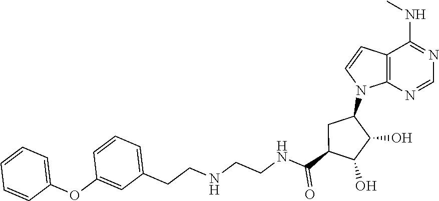 [SMILES: CNc1ncnc2c1ccn2[C@@H]1C[C@H](C(=O)NCCNCCc2cccc(Oc3ccccc3)c2)[C@@H](O)[C@H]1O]

According to general procedure F. Peptidic coupling (356): yield: 15 mg, 28.3% (TFA salt). 1H NMR (400 MHZ, CD₃OD): δ=8.04 (s, 1H), 7.24-7.22 (m, 3H), 7.12 (t, J=8.0 Hz, 1H), 6.98 (t, J=6.8 Hz, 1H), 6.88-6.76 (m, 3H), 6.76 (s, 1H), 6.68-6.65 (m, 1H), 6.48 (m, 1H), 4.92 (m, 1H), 4.19-4.16 (m, 1H), 4.13-4.11 (m, 1H), 3.26-3.24 (m, 2H), 2.96 (s, 3H), 2.77-2.65 (m, 7H), 2.39-2.35 (m, 1H), 2.09-2.07 (m, 1H). MS calc. for C₂₉H₃₄N₆O₄. MS calc. [M+H]⁺: 530.3, found: 531.3 [M+H]⁺. Purified by method C(NH₄HCO₃ buffer). UV-purity >85%.

### (1S,2R,3R,5R)-3-(((2,2-difluoro-3-((2-(naphthalen-2-yl)ethyl) amino)propyl) amino)methyl)-5-(4-(methylamino)-7H-pyrrolo[2,3-a]pyrimidin-7-yl) cyclopentane-1,2-diol (357)

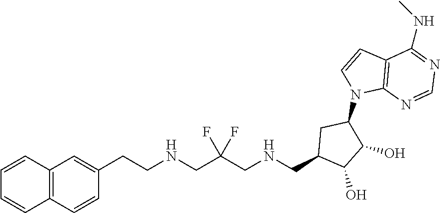 [SMILES: CNc1ncnc2c1ccn2[C@@H]1C[C@H](CNCC(F)(F)CNCCc2ccc3ccccc3c2)[C@@H](O)[C@H]1O]

According to general procedure N. Deprotection (357): yield: 13.3 mg, 48% (TFA salt). ¹H NMR (400 MHZ, CD₃OD): δ=8.25 (s, 1H), 7.90-7.83 (m, 3H), 7.77 (s, 1H), 7.53-7.47 (m, 3H), 7.43 (dd, J=1.6, 8.4 Hz, 1H), 6.89 (brs, 1H), 5.04-4.86 (m, 1H), 4.32-4.28 (m, 1H), 4.06-4.02 (m, 1H), 3.89-3.65 (m, 4H), 3.46-3.44 (m, 2H), 3.23-3.09 (m, 7H), 2.40-2.36 (m, 2H), 1.78-1.72 (m, 1H). MS calc. for C₂₈H₃₄F₂N₆O₂. MS calc. [M+H]⁺: 524.2, found: 525.3 [M+H]⁺. Purified by method C(TFA buffer). UV-purity >92%.

### tert-butyl ((E)-5-((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) pent-4-en-1-yl)(phenethyl)carbamate (358) & (1R,2S,3R,5R)-3-(4-(methylamino)-7H-pyrrolo[2,3-a]pyrimidin-7-yl)-5-((E)-5-(phenethylamino) pent-1-en-1-yl) cyclopentane-1,2-diol (359)

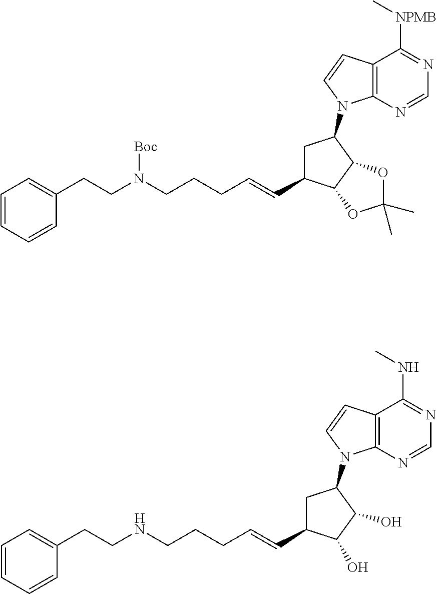

A solution of 122 (7 mg, 0.01 mmol) in TFA (6 mL) was stirred at room temperature for 16 hrs. The mixture was concentrated at low temperature to give 358 (5 mg, crude) as a clear oil. MS Calc.: 531.3; MS Found: 532.0 [M+H⁺]. To a solution of 358 (5 mg, 0.01 mmol) in MeOH (2 mL) was added K₂CO₃ (4 mg), and the solution was stirred at room temperature for 1 hr. The mixture was concentrated to dryness and the residue was purified by prep-HPLC(TFA buffer) to give 359 (2.6 mg, yield: 60%) as a white solid. ¹H NMR (400 MHZ, CD₃OD): δ=8.13 (s, 1H), 7.41 (d, J=3.6 Hz, 1H), 7.25 (t, J=8.4 Hz, 2H), 7.17 (t, J=7.6 Hz, 3H), 6.76 (brs, 1H), 5.50-5.40 (m, 2H), 4.97-4.87 (m, 1H), 4.24 (t, J=6.4 Hz, 1H), 3.85 (t, J=6.0 Hz, 1H), 3.18-2.87 (m, 10H), 2.28-2.12 (m, 3H), 1.74-1.67 (m, 3H). MS Calc.: 435.3; MS Found: 436.3 [M+H⁺].

### (1R,2S,3R,5R)-3-(4-(methylamino)-7H-pyrrolo[2,3-d]pyrimidin-7-yl)-5-((3-(((2-(naphthalen-2-yl)ethyl) amino) methyl) azetidin-1-yl)methyl) cyclopentane-1,2-diol (360)

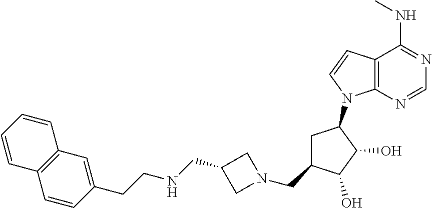

According to general procedure N. Deprotection (360): yield: 14.00 mg, 21% (TFA salt). ¹H NMR (400 MHZ, CD₃OD): δ=8.25 (s, 1H), 7.90-7.78 (m, 4H), 7.53-7.42 (m, 4H), 6.88 (s, 1H), 5.04-4.98 (m, 1H), 4.42 (s, 2H), 4.31 (t, J=6.4 Hz, 1H), 4.17-4.08 (m, 3H), 3.57-3.38 (m, 7H), 3.22-3.170 (m, 5H), 2.46-2.29 (m, 2H), 1.86-1.77 (m, 1H). MS calc. for C₂₉H₃₆N₆O₂. MS calc. [M+H]⁺: 500.3, found: 501.3 [M+H]⁺. Purified by method C(TFA buffer). UV-purity >96%.

### tert-butyl (7-((3αS,4R,6R,6αR)-6-(((3-((tert-butoxycarbonyl)(3-phenoxyphenethyl) amino)propyl)(isopropyl) amino)methyl)-2,2-dimethyltetrahydro-4H-cyclopenta[d][1,3]dioxol-4-yl)-7H-pyrrolo[2,3-d]pyrimidin-4-yl)(methyl)carbamate (361) & (1S,2R,3R,5R)-3-((isopropyl(3-((3-phenoxyphenethyl) amino)propyl) amino)methyl)-5-(4-(methylamino)-7H-pyrrolo[2,3-d]pyrimidin-7-yl) cyclopentane-1,2-diol (362)

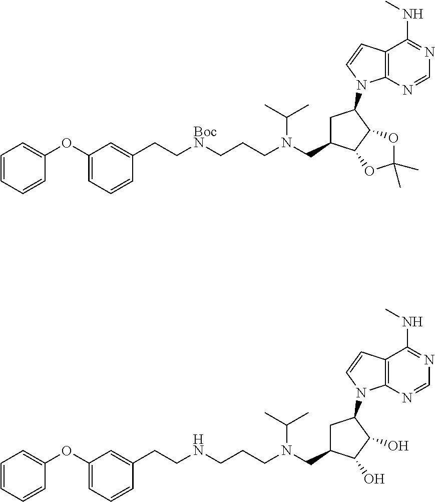 [SMILES: CNc1ncnc2c1ccn2[C@@H]1C[C@H](CN(CCCN(CCc2cccc(Oc3ccccc3)c2)C(=O)OC(C)(C)C)C(C)C)[C@H]2OC(C)(C)O[C@H]21 ; CNc1ncnc2c1ccn2[C@@H]1C[C@H](CN(CCCNCCc2cccc(Oc3ccccc3)c2)C(C)C)[C@@H](O)[C@H]1O]

According to general procedure B. Reductive amination (361): two-step yield: 30 mg, 13.5%. C₄₁H₅₆N₆O₅ (712.94 g/mol). MS calc. [M+H]⁺: 712.40, found: 713.40. Prep HPLC(NH₄OAc buffer). According to general procedure N. Deprotection (362): yield: 10.00 mg, 41.6% (TFA salt). ¹H NMR (400 MHZ, CD₃OD): δ=8.24 (s, 1H), 7.42-7.34 (m, 4H), 7.18-7.16 (m, 1H), 7.09-7.07 (m, 1H), 7.03-7.00 (m, 3H), 6.94-6.90 (m, 1H), 6.80 (s, 1H), 5.02-4.97 (m, 1H), 4.34-4.33 (m, 1H), 4.19-4.15 (m, 1H), 3.87-3.82 (m, 1H), 3.46-3.30 (m, 6H), 3.30-3.24 (m, 5H), 3.06-3.01 (m, 2H), 2.56-2.54 (m, 2H), 2.28 (m, 2H), 1.87-1.84 (m, 1H), 1.46-1.42 (m, 6H). MS calc. for C₃₃H₄₄N₆O₃. [M+H]⁺: 572.3, found: 573.4 [M+H]⁺. Purified by method C(TFA buffer). UV-purity >95%.

### tert-butyl (7-((3αS,4R,6R,6αR)-6-(((3-((tert-butoxycarbonyl)(3-phenoxyphenethyl) amino)propyl)(ethyl) amino)methyl)-2,2-dimethyltetrahydro-4H-cyclopenta[d][1,3]dioxol-4-yl)-7H-pyrrolo[2,3-d]pyrimidin-4-yl)(methyl)carbamate (363) & (1S,2R,3R,5R)-3-((ethyl(3-((3-phenoxyphenethyl) amino)propyl) amino)methyl)-5-(4-(methylamino)-7H-pyrrolo[2,3-d]pyrimidin-7-yl) cyclopentane-1,2-diol (364)

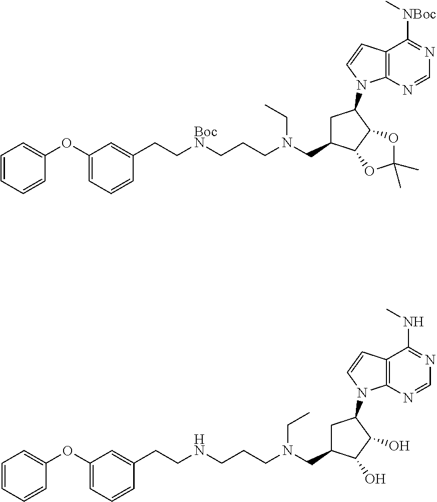 [SMILES: CCN(CCCN(CCc1cccc(Oc2ccccc2)c1)C(=O)OC(C)(C)C)C[C@H]1C[C@@H](n2ccc3c(N(C)C(=O)OC(C)(C)C)ncnc32)[C@@H]2OC(C)(C)O[C@H]12 ; CCN(CCCNCCc1cccc(Oc2ccccc2)c1)C[C@H]1C[C@@H](n2ccc3c(NC)ncnc32)[C@H](O)[C@@H]1O]

According to general procedure B. Reductive amination (363): two-step yield: 30 mg, 13.5%. C₄₀H₅₄N₆O₅ (698.91 g/mol). MS calc. [M+H]⁺: 698.40, found: 699.40. Prep HPLC(NH₄OAc buffer). According to general procedure N. Deprotection (364): yield: 10.00 mg, 42.7% (OAc salt). 1H NMR (400 MHZ, CD₃OD) δ:8.15 (s, 1H), 7.40-7.27 (m, 3H), 7.19 (d, J=3.6 Hz, 1H), 7.10 (t, J=8.0 Hz, 1H), 7.01-6.95 (m, 3H), 6.94 (t, J=2.0 Hz, 1H), 6.85 (dd, J=8.0, 2.0 Hz, 1H), 6.57 (d, J=7.6 Hz, 1H), 4.87-4.83 (m, 1H, overlap with water peak), 4.35 (t, J=6.8 Hz, 1H), 3.90 (t, J=5.4 Hz, 1H), 3.32-3.15 (m, 4H), 3.07 (s, 3H), 2.95 (t, J=7.6 Hz, 2H), 2.83-2.55 (m, 6H), 2.34-2.30 (m, 2H), 1.90-1.87 (m, 3H), 1.11 (t, J=6.8 Hz, 3H). MS calc. for C₃₂H₄₂N₆O₃. [M+H]⁺: 559.3, found: 559.4 [M+H]⁺. Purified by method C(NH₄OAc buffer). UV-purity >97%.

### tert-butyl (7-((3αS,4R,6R,6αR)-6-(((3-((tert-butoxycarbonyl)(3-phenoxyphenethyl) amino)propyl)(cyclopropylmethyl) amino)methyl)-2,2-dimethyltetrahydro-4H-cyclopenta[d][1,3]dioxol-4-yl)-7H-pyrrolo[2,3-d]pyrimidin-4-yl)(methyl)carbamate (365) & (1S,2R,3R,5R)-3-(((cyclopropylmethyl)(3-((3-phenoxyphenethyl) amino)propyl) amino)methyl)-5-(4-(methylamino)-7H-pyrrolo[2,3-d]pyrimidin-7-yl) cyclopentane-1,2-diol (366)

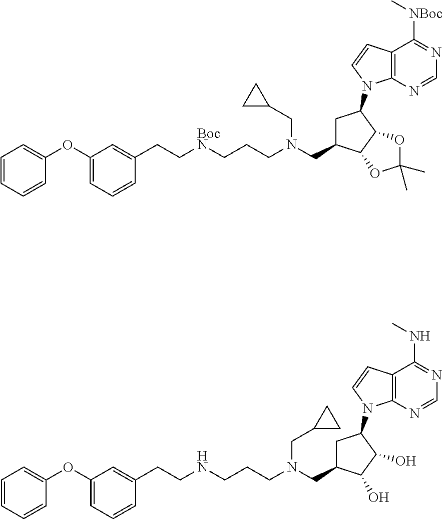 [SMILES: CN(C(=O)OC(C)(C)C)c1ncnc2c1ccn2[C@@H]1C[C@H](CN(CCCN(CCc2cccc(Oc3ccccc3)c2)C(=O)OC(C)(C)C)CC2CC2)[C@H]2OC(C)(C)O[C@H]21 ; CNc1ncnc2c1ccn2[C@@H]1C[C@H](CN(CCCNCCc2cccc(Oc3ccccc3)c2)CC2CC2)[C@@H](O)[C@H]1O]

According to general procedure B. Reductive amination (365): two-step yield: 30 mg, 13.3%. C₄₂H₅₆N₆O₅ (724.95 g/mol). MS calc. [M+H]⁺: 724.40, found: 725.40. Prep HPLC(NH₄OAc buffer). According to general procedure N. Deprotection (366): yield: 10.00 mg, 40.7% (TFA salt). ¹H NMR (400 MHZ, CD₃OD): δ=8.08 (s, 1H), 7.27-7.19 (m, 4H), 7.02 (t, J=7.6 Hz, 1H), 6.95-6.84 (m, 4H), 6.77 (dd, J=8.0, 2.0 Hz, 1H), 6.59 (d, J=3.2 Hz, 1H), 4.83-4.76 (m, 1H), 4.22 (t, J=6.0 Hz, 1H), 4.02 (t, J=6.4 Hz, 1H), 3.40-3.33 (m, 1H), 3.20-3.18 (m, 5H), 3.08 (t, J=7.2 Hz, 2H), 3.01 (s, 3H), 3.01-2.95 (m, 3H), 2.92-2.86 (m, 2H), 2.37-2.33 (m, 2H), 2.06-2.05 (m, 2H), 1.02-0.98 (m, 1H), 0.67-0.65 (m, 2H), 0.35-0.34 (m, 2H). MS calc. for C₃₄H₄₄N₆O₃. [M+H]⁺: 584.3, found: 585.4 [M+H]⁺. Purified by method C(TFA buffer). UV-purity >99%.

### (1S,2R,3R,5R)-3-((2-methyl-1-(3-((3-phenoxyphenethyl) amino)propyl) hydrazineyl)methyl)-5-(4-(methylamino)-7H-pyrrolo[2,3-d]pyrimidin-7-yl) cyclopentane-1,2-diol (367)

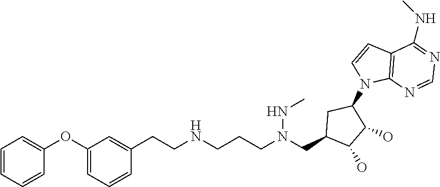 [SMILES: CNc1ncnc2c1ccn2[C@@H]1C[C@H](CN(CCCNCCc2cccc(Oc3ccccc3)c2)NC)[C@@H](O)[C@H]1O]

According to general procedure N. Deprotection (367): yield: 10.00 mg, 35.7% (TFA salt). ¹H NMR (400 MHZ, CD₃OD): δ=8.24 (s, 1H), 7.55 (d, J=3.2 Hz, 1H), 7.39-7.32 (m, 3H), 7.16-7.14 (m, 1H), 7.12 (m, 1H), 7.00-6.96 (m, 3H), 6.91-6.88 (m, 2H), 5.09-5.06 (m, 1H), 4. 37 (d, J=6.8 Hz, 1H), 4.07 (d, J=5.6 Hz, 1H), 3.29-3.27 (m, 3H), 3.19-3.03 (m, 11H), 2.89-2.88 (m, 2H), 2.45-2.39 (m, 2H), 1.96-2.11 (m, 2H), 1.74-1.69 (m, 1H). MS calc. for C₃₁H₄₁N₇O₃. [M+H]⁺: 559.3, found: 560.4 [M+H]⁺. Purified by method C(TFA buffer). UV-purity >99%.

### (1R,2S,3R,5R)-3-(4-(methylamino)-7H-pyrrolo[2,3-a]pyrimidin-7-yl)-5-(((3-((2-(naphthalen-2-yl)ethyl) amino)propyl) amino) methyl) cyclopentane-1,2-diol (368)

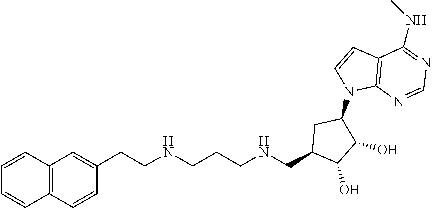 [SMILES: CNc1ncnc2c1ccn2[C@@H]1C[C@H](CNCCCNCCc2ccc3ccccc3c2)[C@@H](O)[C@H]1O]

According to general procedure N. Deprotection (368): yield: 10.00 mg, 42.7% (TFA salt). ¹H NMR (400 MHZ, D₂O): 8=8.13 (s, 1H), 7.78-7.72 (m, 3H), 7.67 (s, 1H), 7.40-7.36 (m, 3H), 7.31 (dd, J=8.4, 2.0 Hz, 1H), 6.76 (brs, 1H), 4.93-4.92 (m, 1H), 4.22 (t, J=6.4 Hz, 1H), 4.01 (t, J=6.0 Hz, 1H), 3.31-3.21 (m, 3H), 3.17-3.07 (m, 10H), 2.40-2.32 (m, 2H), 2.09-2.06 (m, 2H), 1.74-1.72 (m, 1H). MS calc. for C₂₈H₃₆NO₂. [M+H]⁺: 448.3, found: 489.3 [M+H]⁺. Purified by method C(TFA buffer). UV-purity >97%.

### tert-butyl (4-(4-chlorophenoxy)-3-fluorophenethyl)(3-((((3αR,4R,6R,6αS)-2,2-dimethyl-6-(4-(methylamino)-7H-pyrrolo[2,3-d]pyrimidin-7-yl) tetrahydro-4H-cyclopenta[d][1,3]dioxol-4-yl)methyl) amino)propyl)carbamate (369) & (1S,2R,3R,5R)-3-(((3-((4-(4-chlorophenoxy)-3-fluorophenethyl) amino)propyl) amino)methyl)-5-(4-(methylamino)-7H-pyrrolo[2,3-a]pyrimidin-7-yl) cyclopentane-1,2-diol (370)

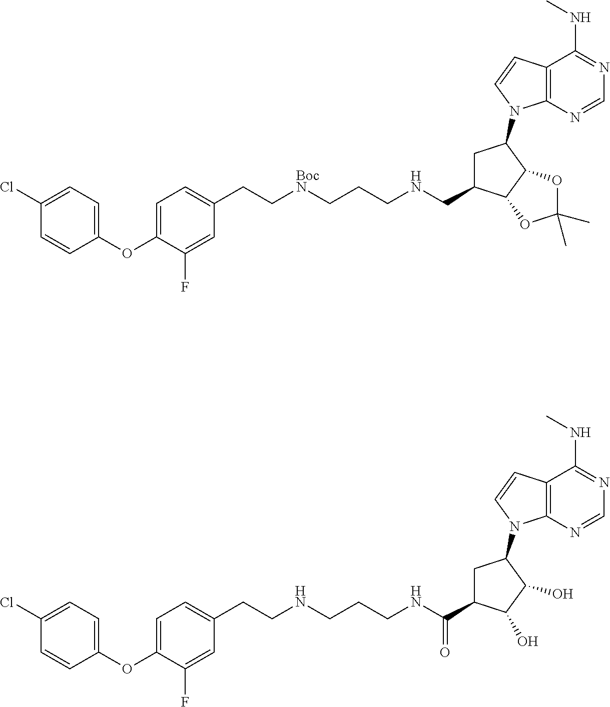 [SMILES: CNc1ncnc2c1ccn2[C@@H]1C[C@H](CNCCCN(CCc2ccc(Oc3ccc(Cl)cc3)c(F)c2)C(=O)OC(C)(C)C)[C@H]2OC(C)(C)O[C@H]21 ; CNc1ncnc2c1ccn2[C@@H]1C[C@H](C(=O)NCCCNCCc2ccc(Oc3ccc(Cl)cc3)c(F)c2)[C@@H](O)[C@H]1O]

According to general procedure B. Reductive amination (369): yield: 30 mg, 44%. C38H48ClFN6O5 (723.29 g/mol). Prep HPLC(TFA buffer). According to general procedure N. Deprotection (370): yield: 20.00 mg, 83.0% (TFA salt). ¹H NMR (400 MHZ, DMSO-d₆): δ=9.13 (brs, 1H), 9.00 (brs, 2H), 8.75 (brs, 2H), 8.32 (s, 1H), 7.55 (brs, 1H), 7.44-7.35 (m, 3H), 7.21 (t, J=8.0 Hz, 1H), 7.16-7.14 (m, 1H), 6.99-6.97 (m, 2H), 6.85 (brs, 1H), 4.95-4.89 (m, 1H), 4.18 (t, J=6.4 Hz, 1H), 3.90 (t, J=6.4 Hz, 1H), 3.15-2.94 (m, 13H), 2.38-2.25 (m, 2H), 2.03-1.96 (m, 2H), 1.67-1.59 (m, 1H). MS calc. for C₃₀H₃₆ClFN₆O₃. [M+H]⁺: 582.3, found: 583.3 [M+H]⁺. Purified by method C (TFA buffer). UV-purity >98%.

### tert-butyl (3-((((3αR,4R,6R,6αS)-2,2-dimethyl-6-(4-(methylamino)-7H-pyrrolo[2,3-a]pyrimidin-7-yl) tetrahydro-4H-cyclopenta[d][1,3]dioxol-4-yl)methyl) amino)propyl)(3-(phenylthio)phenethyl)carbamate (371) & (1R,2S,3R,5R)-3-(4-(methylamino)-7H-pyrrolo[2,3-a]pyrimidin-7-yl)-5-(((3-((3-(phenylthio)phenethyl) amino)propyl) amino) methyl) cyclopentane-1,2-diol (372)

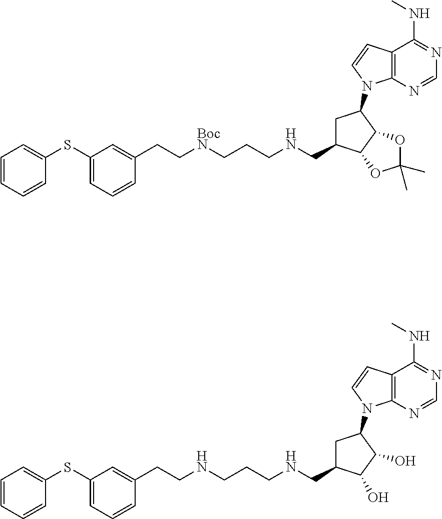 [SMILES: CNc1ncnc2c1ccn2[C@@H]1C[C@H](CNCCCN(CCc2cccc(Sc3ccccc3)c2)C(=O)OC(C)(C)C)[C@H]2OC(C)(C)O[C@H]21 ; CNc1ncnc2c1ccn2[C@@H]1C[C@H](CNCCCNCCc2cccc(Sc3ccccc3)c2)[C@@H](O)[C@H]1O]

According to general procedure B. Reductive amination (371): yield: 30 mg, 16.8%. C38H50N6O4S (686.92 g/mol). MS calc. [M+H]⁺: 686.40, found: 687.40. Prep HPLC(TFA buffer). According to general procedure N. Deprotection (372): yield: 15.00 mg, 63.9% (TFA salt). 1H NMR (400 MHZ, D₂₀): 8=8.25 (s, 1H), 7.52-7.49 (m, 1H), 7.37-7.29 (m, 7H), 7.21 (t, J=6.8 Hz, 2H), 6.88 (s, 1H), 5.05-5.04 (m, 1H), 4.36-4.33 (t, J=6.4 Hz, 1H), 4.14-4.11 (t, J=6.0 Hz, 1H), 3.39-3.34 (m, 1H), 3.29-3.16 (m, 10H), 3.01-2.97 (m, 2H), 2.51-2.44 (m, 2H), 2.19-2.17 (m, 2H), 1.86-1.83 (m, 1H). MS calc. for C30H38N6O2S. [M+H]⁺: 546.3, found: 547.4 [M+H]⁺. Purified by method C (TFA buffer). UV-purity >95%.

### tert-butyl (3-((((3αR,4R,6R,6αS)-2,2-dimethyl-6-(4-(methylamino)-7H-pyrrolo[2,3-a]pyrimidin-7-yl) tetrahydro-4H-cyclopenta[d][1,3]dioxol-4-yl)methyl) amino)propyl)(3-phenethylphenethyl)carbamate (373) & (1R,2S,3R,5R)-3-(4-(methylamino)-7H-pyrrolo[2,3-d]pyrimidin-7-yl)-5-(((3-((3-phenethylphenethyl) amino)propyl) amino) methyl) cyclopentane-1,2-diol (374)

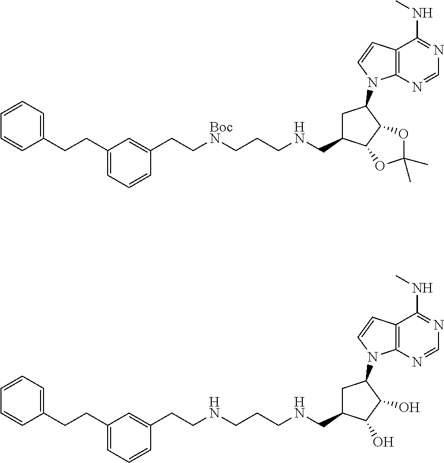 [SMILES: CNc1ncnc2c1ccn2[C@@H]1C[C@H](CNCCCN(CCc2cccc(CCc3ccccc3)c2)C(=O)OC(C)(C)C)[C@H]2OC(C)(C)O[C@H]21 ; CNc1ncnc2c1ccn2[C@@H]1C[C@H](CNCCCNCCc2cccc(CCc3ccccc3)c2)[C@@H](O)[C@H]1O]

According to general procedure B. Reductive amination (373): yield: 30 mg, 44%. C₄₀H₅₄N₆O₄ (682.91 g/mol). MS calc. [M+H]⁺: 682.4, found: 683.1. According to general procedure N. Deprotection (374): yield: 16.00 mg, 67.0% (TFA salt). ¹H NMR (400 MHZ, CD₃OD): δ=8.24 (s, 1H), 7.49 (s, 1H), 7.26-7.14 (m, 9H), 6.86 (s, 1H), 5.04-5.02 (m, 1H), 4.34 (t, J=6.4 Hz, 1H), 4.13 (t, J=6.4 Hz, 1H), 3.39-3.19 (m, 11H), 3.01-2.97 (m, 2H), 2.91 (s, 4H), 2.52-2.44 (m, 2H), 2.22-2.14 (m, 2H), 1.86-1.83 (m, 1H). MS calc. for C₃₂H₄₂N₆O₂. [M+H]⁺: 542.3, found: 547.4 [M+H]⁺. Purified by method C(TFA buffer). UV-purity >97%.

### (1S,2R,3R,5R)-3-((ethyl(3-((2-(naphthalen-2-yl)ethyl) amino)propyl) amino)methyl)-5-(4-(methylamino)-7H-pyrrolo[2,3-d]pyrimidin-7-yl) cyclopentane-1,2-diol (375)

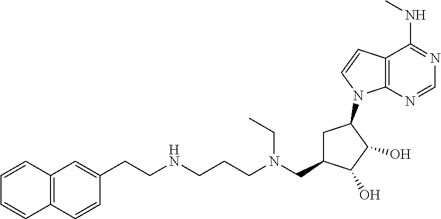 [SMILES: CCN(CCCNCCc1ccc2ccccc2c1)C[C@H]1C[C@@H](n2ccc3c(NC)ncnc32)[C@H](O)[C@@H]1O]

According to general procedure N. Deprotection (375): yield: 10.00 mg, 43.0% (TFA salt). ¹H NMR (400 MHZ, D₂O): 8=8.13 (s, 1H), 7.79-7.72 (m, 3H), 7.68 (s, 1H), 7.40-7.37 (m, 3H) 7.34-7.31 (m, 1H), 6.76 (m, 1H), 4.90 (m, 1H), 4.22-4.19 (m, 1H), 4.03-4.00 (m, 1H), 3.33-3.23 (m, 8H), 3.12-3.08 (m, 7H), 2.41-2.38 (m, 2H), 2.12-2.09 (m, 2H), 1.75 (m, 1H), 1.29-1.26 (m, 3H). MS calc. for C₃₀H₄₀N₆O₂. [M+H]⁺: 516.3, found: 517.5 [M+H]⁺. Purified by method C(TFA buffer). UV-purity >94%.

### tert-butyl (3-((((3αR,4R,6R,6αS)-2,2-dimethyl-6-(4-(methylamino)-7H-pyrrolo[2,3-a]pyrimidin-7-yl) tetrahydro-4H-cyclopenta[d][1,3]dioxol-4-yl)methyl)(2,2,2-trifluoroethyl) amino)propyl)(2-(naphthalen-2-yl)ethyl)carbamate (376) & (1R,2S,3R,5R)-3-(4-(methylamino)-7H-pyrrolo[2,3-d]pyrimidin-7-yl)-5-(((3-((2-(naphthalen-2-yl)ethyl) amino)propyl)(2,2,2-trifluoroethyl) amino) methyl) cyclopentane-1,2-diol (377)

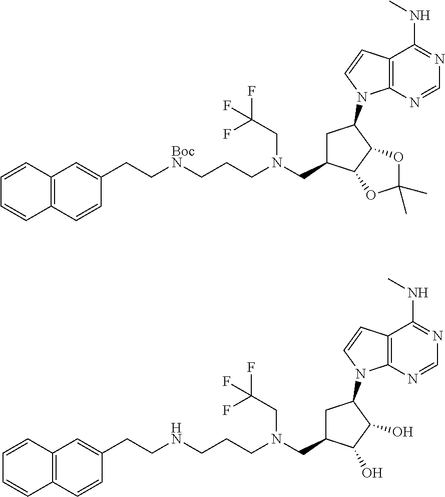 [SMILES: CNc1ncnc2c1ccn2[C@@H]1C[C@H](CN(CCCN(CCc2ccc3ccccc3c2)C(=O)OC(C)(C)C)CC(F)(F)F)[C@H]2OC(C)(C)O[C@H]21 ; CNc1ncnc2c1ccn2[C@@H]1C[C@H](CN(CCCNCCc2ccc3ccccc3c2)CC(F)(F)F)[C@@H](O)[C@H]1O]

To a solution of compound 129 (50 mg, 0.08 mmol) and TEA (0.1 mL) in DMF (2.00 mL) was added 2,2,2-trifluoroethyl trifluoromethanesulfonate (37 mg, 0.16 mmol), and the mixture was stirred at 60° C. for 1 hr. Then The mixture was purified by prep-HPLC(TFA buffer) to give compound 376 (10 mg, yield: 17.8%) as a white solid. According to general procedure Q. Deprotection (377): yield: 1.50 mg, 18.8% (TFA salt). ¹H NMR (400 MHZ, D₂O): 8=8.22 (s, 1H), 7.89-7.84 (m, 3H), 7.83 (s, 1H), 7.76-7.51 (m, 3H) 7.41 (dd, J=8.4, 1.6 Hz, 1H), 6.85 (brs, 1H), 5.09-5.02 (m, 1H), 4.40-4.36 (m, 1H), 3.98-3.96 (m, 1H), 3.50-3.36 (m, 2H), 3.29-3.12 (m, 9H), 2.91-2.69 (m, 4H), 2.40-2.35 (m, 1H), 2.30-2.28 (m, 1H), 1.98-1.86 (m, 2H) 1.71-1.62 (m, 1H). MS calc. for C₃₀H₃₇F₃N₆O₂. [M+H]⁺: 570.3, found: 571.4 [M+H]⁺. Purified by method C(TFA buffer). UV-purity >97%.

### tert-butyl (3-((((3αR,4R,6R,6αS)-6-(5-ethyl-4-((4-methoxybenzyl)amino)-7H-pyrrolo[2,3-d]pyrimidin-7-yl)-2,2-dimethyltetrahydro-4H-cyclopenta[d][1,3]dioxol-4-yl)methyl) amino)propyl)(4-(trifluoromethyl)phenethyl)carbamate (378) & (1R,2S,3R,5R)-3-(4-amino-5-ethyl-7H-pyrrolo[2,3-d]pyrimidin-7-yl)-5-(((3-((4-(trifluoromethyl)phenethyl) amino)propyl) amino) methyl) cyclopentane-1,2-diol (379)

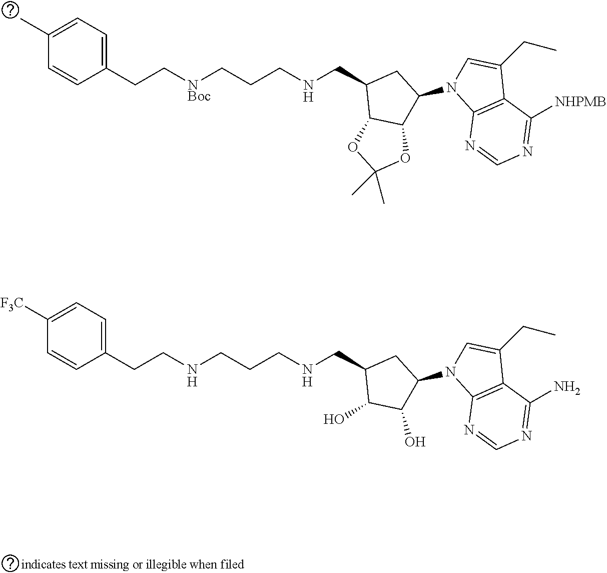

According to general procedure D. Reductive amination (378): yield: 100.00 mg, 37%. APCI: calc. [M+H]⁺: 780.93, found: 781.6. ¹H-NMR: (400 MHZ, DMSO-d₆)}=8.06 (s, 1H), 7.68-7.57 (m, 2H), 7.45-7.36 (m, 2H), 7.30-7.22 (m, 2H), 7.10 (s, 1H), 6.91 (t, J=6.1 Hz, 1H), 6.87-6.80 (m, 2H), 4.97-4.88 (m, 1H), 4.87-4.80 (m, 1H), 4.69-4.61 (m, 2H), 4.51-4.40 (m, 1H), 3.70 (s, 3H), 3.44-3.25 (m, 5H), 3.23-3.10 (m, 2H), 2.83 (q, J=7.3 Hz, 2H), 2.81-2.66 (m, 2H), 2.28-2.10 (m, 2H), 2.02-1.87 (m, 2H), 1.68-1.54 (m, 2H), 1.44 (s, 3H), 1.35 and 1.25 (s, 9H), 1.22 (t, J=7.4 Hz, 3H), 1.20 (s, 3H) ppm. According to general procedure O. Deprotection (379): The crude product was purified by preparative HPLC according to method D. yield: 76.00 mg, 79% (2 TFA salt). APCI-MS (+) m/z for C₂₆H₃₅F3N₆O₂: calc.: 520.60; found: 521.3 and 522.4. ¹H-NMR: (400 MHZ, DMSO-d₆) δ=8.91 (bs, 2H), 8.73 (bs, 2H), 8.35 (s, 1H), 7.72 (d, J=8.1 Hz, 2H), 7.51 (d, J=8.0 Hz, 2H), 7.45 (s, 1H), 4.97-4.90 (m, 1H), 4.18-4.14 (m, 1H), 3.89-3.84 (m, 1H), 3.26-3.17 (m, 3H), 3.05-2.97 (m, 7H), 2.84 (q, J=7.1 Hz, 2H), 2.30-2.22 (m, 2H), 2.02-1.94 (m, 2H), 1.64-1.56 (m, 1H), 1.23 (t, J=7.4 Hz, 3H) ppm. TR=11.87 min; UV-purity at 210, 230, 254, 242 and 275 nm >97%.

### tert-butyl (3-((((3αR,4R,6R,6αS)-6-(5-ethyl-4-((4-methoxybenzyl)amino)-7H-pyrrolo[2,3-d]pyrimidin-7-yl)-2,2-dimethyltetrahydro-4H-cyclopenta[d][1,3]dioxol-4-yl)methyl) amino)propyl)(4-fluorophenethyl)carbamate (380) & (1R,2S,3R,5R)-3-(4-amino-5-ethyl-7H-pyrrolo[2,3-d]pyrimidin-7-yl)-5-(((3-((4-fluorophenethyl) amino)propyl) amino) methyl) cyclopentane-1,2-diol (381)

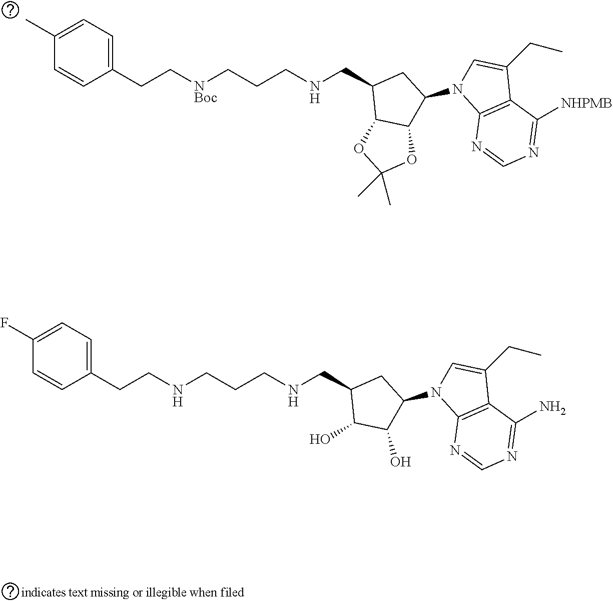

According to general procedure D. Reductive amination (380): yield: 140.00 mg, 62%. APCI: calc. [M+H]⁺: 730.93, found: 731.6 and 732.6. ¹H-NMR: (400 MHZ, DMSO-d₆) δ=8.06 (s, 1H), 7.28-7.24 (m, 2H), 7.22-7.15 (m, 2H), 7.12-7.03 (m, 3H), 6.89 (t, J=6.2 Hz, 1H), 6.86-6.82 (m, 2H), 4.94-4.87 (m, 1H), 4.85-4.82 (m, 1H), 4.65 (d, J=6.1 Hz, 2H), 4.45-4.41 (m, 1H), 3.70 (s, 3H), 3.40-3.24 (m, 6H), 3.19-3.09 (m, 2H), 2.83 (q, J=7.2 Hz, 2H), 2.75-2.71 (m, 2H), 2.26-2.21 (m, 2H), 1.98-1.91 (m, 1H), 1.62-1.54 (m, 2H), 1.44 (s, 3H), 1.36 and 1.32 (s, 9H), 1.22 (t, J=7.4 Hz, 3H), 1.20 (s, 3H) ppm. According to general procedure O. Deprotection (381): The crude product was purified by preparative HPLC according to method D. yield: 98.00 mg, 73% (2 TFA salt). APCI-MS (+) m/z for C₂₅H₃₅FN₆O₂: calc.: 470.59; found: 471.4 and 472.4. ¹H-NMR: (400 MHZ, DMSO-d₆) δ=8.81 (bs, 2H), 8.68 (bs, 2H), 8.34 (s, 1H), 7.43 (s, 1H), 7.33-7.28 (m, 2H), 7.21-7.15 (m, 2H), 4.96-4.90 (m, 1H), 4.18-4.14 (m, 1H), 3.89-3.85 (m, 1H), 3.23-3.12 (m, 3H), 3.09-2.98 (m, 5H), 2.94-2.88 (m, 2H), 2.84 (q, J=7.3 Hz, 2H), 2.30-2.20 (m, 2H), 2.01-1.93 (m, 2H), 1.63-1.57 (m, 1H), 1.23 (t, J=7.4 Hz, 3H) ppm. TR=10.56 min; UV-purity at 210, 230, 254, 242 and 275 nm >97%).

### tert-butyl (2,4-difluorophenethyl)(3-((((3αR,4R,6R,6αS)-6-(5-ethyl-4-((4-methoxybenzyl) amino)-7H-pyrrolo[2,3-a]pyrimidin-7-yl)-2,2-dimethyltetrahydro-4H-cyclopenta[d][1,3]dioxol-4-yl)methyl) amino)propyl)carbamate (382) & (1R,2S,3R,5R)-3-(4-amino-5-ethyl-7H-pyrrolo[2,3-d]pyrimidin-7-yl)-5-(((3-((2,4-difluorophenethyl) amino)propyl) amino) methyl) cyclopentane-1,2-diol (383)

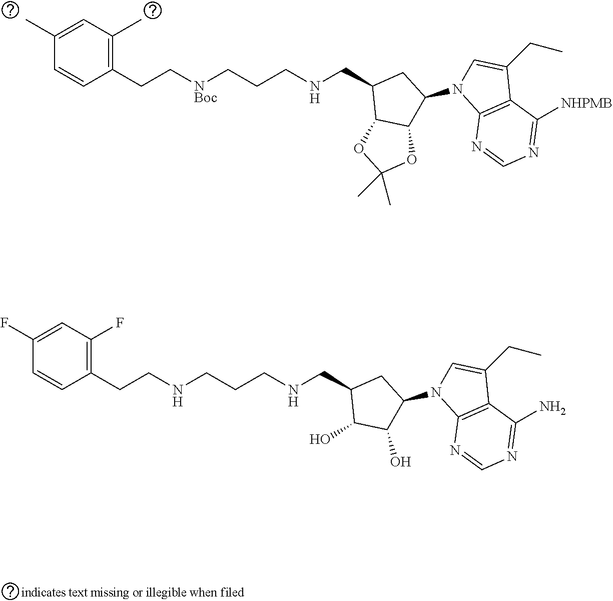

According to general procedure D. Reductive amination (382): yield: 60.00 mg, 40%. APCI-MS (+) m/z for C₄₁H₅₄F2N₆O₅: calc.: 748.92; found: 749.5 and 750.5. ¹H-NMR: (400 MHZ, DMSO-d₆) δ=8.06 (s, 1H), 7.30-7.22 (m, 3H), 7.20-7.12 (m, 1H), 7.09 (s, 1H), 7.03-6.94 (m, 1H), 6.90 (t, J=6.1 Hz, 1H), 6.87-6.81 (m, 2H), 4.93-4.87 (m, 1H), 4.85-4.81 (m, 1H), 4.65 (d, J=6.1 Hz, 2H), 4.41-4.44 (m, 1H), 3.70 (s, 3H), 3.22-3.05 (m, 2H), 2.79-2.72 (m, 2H), 2.83 (q, J=7.3 Hz, 2H), 2.66-2.61 (m, 1H), 2.25-2.09 (m, 2H), 1.99-1.88 (m, 1H), 1.63-1.52 (m, 2H), 1.44 (s, 3H), 1.34 and 1.26 (s, 9H), 1.23 (t, J=7.4 Hz, 3H), 1.20 (s, 3H) ppm; signal of five Protons overlayed by the H₂O-peak. According to general procedure O. Deprotection (383): The crude product was purified by preparative HPLC according to method D. yield: 45.00 mg, 79% (2 TFA salt). APCI-MS (+) m/z for C₂₅H₃₄F2N₆O₂: calc.: 488.58; found: 489.3 and 490.4. ¹H-NMR: (400 MHZ, DMSO-d₆) δ=8.86 (bs, 2H), 8.68 (bs, 2H), 8.35 (s, 1H), 7.43 (s, 1H), 7.45-7.38 (m, 1H), 7.33-7.26 (m, 1H), 7.14-7.09 (m, 1H), 4.98-4.91 (m, 1H), 4.18-4.14 (m, 1H), 3.89-3.85 (m, 1H), 3.23-3.11 (m, 3H), 3.09-2.99 (m, 5H), 2.98-2.93 (m, 2H), 2.85 (q, J=7.4 Hz, 2H), 2.31-2.24 (m, 2H), 2.01-1.93 (m, 2H), 1.64-1.55 (m, 1H), 1.24 (t, J=7.4 Hz, 3H) ppm. TR=10.78 min; UV-purity at 210, 230, 254, 242 and 275 nm >97%.

### (1R,2S,3R,5R)-3-(4-amino-7H-pyrrolo[2,3-a]pyrimidin-7-yl)-5-(3,5-dichlorophenyl) cyclopentane-1,2-diol (384)

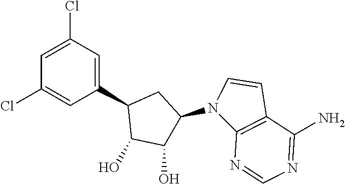 [SMILES: Nc1ncnc2c1ccn2[C@@H]1C[C@H](c2cc(Cl)cc(Cl)c2)[C@@H](O)[C@H]1O]

Following the general procedure for deprotection Q, compound 384 was obtained starting from compound 264 (0.03 g, 0.07 mmol) in a mixture of water and TFA (0.05 M, 2.00 mL, 1:4) after preparative HPLC using method D (water/MeCN; 0.05% TFA) as white foam (16.0 mg, 60%). 1H NMR (400 MHZ, DMSO-d₆) δ 8.37 (s, 1H, H2), 7.81 (d, J=3.6 Hz, 1H, H6), 7.49 (s, 3H, o, p-Ar), 6.96 (d, J=3.6 Hz, 1H, H5), 4.98 (dt, J=11.2, 7.1 Hz, 1H, H1′), 4.22 (t, J=6.6 Hz, 1H, H2′), 4.06 (t, J=7.2 Hz, 1H, H3′), 3.14 (dt, J=12.1, 7.0 Hz, 1H, H4′), 2.37 (dt, J=12.5, 7.4 Hz, 1H, H4′), 2.03 (q, J=12.0 Hz, 1H, H5′). HPLC: t_{R}=15.500 min (Method A). UV-purity at 210 nm=100.0%.

### (1R,2S,3R,5R)-3-(4-amino-7H-pyrrolo[2,3-d]pyrimidin-7-yl)-5-phenylcyclopentane-1,2-diol (385)

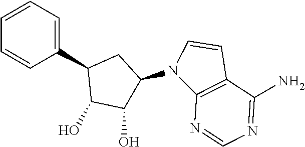 [SMILES: Nc1ncnc2c1ccn2[C@@H]1C[C@H](c2ccccc2)[C@@H](O)[C@H]1O]

Following the general procedure for deprotection Q, compound 385 was obtained starting from compound 266 (0.02 g, 0.003 mmol) in a mixture of water and TFA (0.03 M, 1.00 mL, 1:4) after preparative HPLC using method D (water/MeCN; 0.05% TFA) as white foam (15 mg, 83%). 1H NMR (400 MHZ, DMSO-d₆) δ8.06 (s, 1H, H2), 7.41-7.30 (m, 5H, H5, o, m-Ar), 7.25-7.19 (m, 1H, p-Ar), 6.94 (s, 2H, —CN/2), 6.56 (d, J=3.5 Hz, 1H, H5), 4.97 (d, J=5.9 Hz, 1H, 3′OH), 4.92-4.88 (m, 1H, H1′), 4.87 (d, J=6.3 Hz, 1H, 2′OH), 4.28 (d, J=6.3 Hz, 1H, H2′), 4.13-4.05 (m, 1H, H3′), 3.08 (dt, J=11.8, 7.3 Hz, 1H, H4′), 2.34 (dt, J=12.4, 7.6 Hz, 1H, H5′), 2.01 (q, J=11.9 Hz, 1H, H5′). HPLC: t_{R}=12.771 min (Method A). UV-purity at 210 nm=100.0%.

### (1R,2S,3R,5R)-3-(4-amino-5-bromo-7H-pyrrolo[2,3-a]pyrimidin-7-yl)-5-phenylcyclopentane-1,2-diol (386)

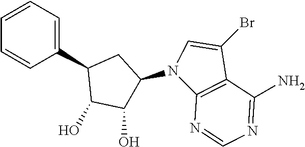 [SMILES: Nc1ncnc2c1c(Br)cn2[C@@H]1C[C@H](c2ccccc2)[C@@H](O)[C@H]1O]

Following the general procedure for deprotection Q, compound 386 was obtained starting from compound 267 (0.02 g, 0.05 mmol) in a mixture of water and TFA (0.05 M, 0.90 mL, 1:4) after preparative HPLC using method D (water/MeCN; 0.05% TFA) as white foam (10.4 mg, 58%). 1H NMR (400 MHZ, DMSO-d₆) δ8.33 (s, 1H, H2), 8.03 (s, 1H, H6), 7.42-7.37 (m, 2H, o-Ar), 7.36-7.31 (m, 2H, m-Ar), 7.27-7.20 (m, 1H, p-Ar), 5.00 (dt, J=11.4, 7.3 Hz, 1H, H1′), 4.28 (t, J=7.1 Hz, 1H, H2′), 4.01 (t, J=6.5 Hz, 1H, H3′), 3.08 (dt, J=11.7, 6.9 Hz, 1H, H4′), 2.39-2.30 (m, 1H, H5′), 2.00 (q, J=11.8 Hz, 1H, H5′). HPLC: TR=13.777 min (Method A). UV-purity at 210 nm=94.6%.

### (1R,2S,3R,5R)-3-(4-amino-7H-pyrrolo[2,3-d]pyrimidin-7-yl)-5-(naphthalen-2-yl) cyclopentane-1,2-diol (387)

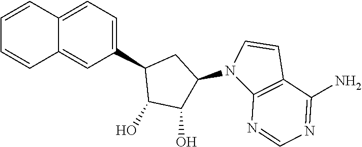 [SMILES: Nc1ncnc2c1ccn2[C@@H]1C[C@H](c2ccc3ccccc3c2)[C@@H](O)[C@H]1O]

Following the general procedure for deprotection Q, compound 387 was obtained starting from compound 268 (0.02 g, 0.04 mmol) in a mixture of water and TFA (0.03 M, 1.40 mL, 1:4) after preparative HPLC using method D (water/MeCN; 0.05% TFA) as white foam (9.10 mg, 57%). 1H NMR (400 MHZ, DMSO-d₆) δ8.40 (s, 1H, H2, adenine), 7.94-7.85 (m, 4H, H1, H4, H5, H8 naphthyl), 7.83 (d, J=3.6 Hz, 1H, H6 adenine), 7.61 (dd, J=8.5, 1.8 Hz, 1H, H3 naphthyl), 7.54-7.44 (m, 2H, H6, H7 naphthyl), 6.97 (d, J=3.6 Hz, 1H, H5 adenine), 5.06 (dt, J=11.3, 7.3 Hz, 1H, H1′), 4.36 (t, J=6.9 Hz, 1H, H2′), 4.16 (t, J=6.6 Hz, 1H, H3′), 3.30 (dt, J=11.6, 6.9 Hz, 1H, H4′), 2.47-2.41 (m, 1H, H5′), 2.16 (q, J=11.7 Hz, 1H, H5′). HPLC: t_{R}=15.349 min (Method A). UV-purity at 210 nm=99.2%.

### (1R,2S,3R,5R)-3-(4-amino-2-chloro-7H-pyrrolo[2,3-d]pyrimidin-7-yl)-5-(naphthalen-2-yl) cyclopentane-1,2-diol (388)

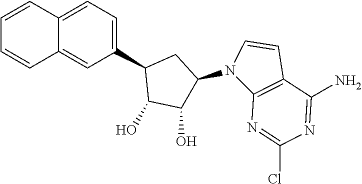 [SMILES: Nc1nc(Cl)nc2c1ccn2[C@@H]1C[C@H](c2ccc3ccccc3c2)[C@@H](O)[C@H]1O]

Following the general procedure for deprotection Q, compound 388 was obtained starting from compound 269 (0.24 g, 0.55 mmol) in a mixture of water and TFA (0.05 M, 10.90 mL, 1:4) after preparative HPLC using method D (water/MeCN; 0.05% TFA) as white foam (18.29 mg, 0.05 mmol). ¹H NMR (400 MHZ, DMSO-d₆) δ 7.92-7.86 (m, 4H, H1, H4, H5, H8 naphthyl), 7.64 (dd, J=8.6, 1.7 Hz, 1H, H6 naphthyl), 7.53-7.46 (m, 4H, —CN H₂, H3, H7 naphthyl), 7.45 (d, J=3.6 Hz, H6 adenine), 6.61 (d, J=3.5 Hz, 1H, H5 adenine), 4.88 (dt, J=10.5, 7.1 Hz, 1H, H1′), 4.32 (t, J=6.7 Hz, 1H, H2′), 4.14 (t, J=6.5 Hz, 1H, H3′), 3.27 (dt, J=11.8, 7.3 Hz, 1H, H4′), 2.47-2.40 (m, 1H, H5′), 2.12 (q, J=11.4 Hz, 1H, H5′). APCI calc.: 394.12; found: 395.20 [M+H]⁺. HPLC: t_{R}=18.589 min (Method A). UV-purity at 210 nm=100.0%.

### (1R,2S,3R,5R)-3-(4-amino-7H-pyrrolo[2,3-d]pyrimidin-7-yl)-5-(pyridin-4-yl) cyclopentane-1,2-diol (389)

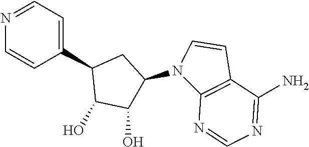 [SMILES: Nc1ncnc2c1ccn2[C@@H]1C[C@H](c2ccncc2)[C@@H](O)[C@H]1O]

Following the general procedure for deprotection Q, compound 389 was obtained starting from compound 270 (0.02 g, 0.06 mmol) in a mixture of water and TFA (0.05 M, 10.90 mL, 1:4) after preparative HPLC using method E (water/MeCN; 0.05% TFA) as white foam (16.20 mg, 89%). 1H NMR (400 MHZ, DMSO-d₆) δ8.83-8.79 (m, 2H, H2, H6 pyridine), 8.41 (s, 1H, H2 adenine), 7.96-7.92 (m, 2H, H3, H5 pyridine), 7.80 (d, J=3.6 Hz, 1H, H6 adenine), 6.99 (d, J=3.6 Hz, 1H, H5 adenine), 5.05 (dt, J=11.0, 7.3 Hz, 1H, H1′), 4.26 (t, J=6.3 Hz, 1H, H2′), 4.17 (t, J=7.1 Hz, 1H, H3′), 3.38 (dt,)=11.8, 7.2 Hz, 1H, H4′), 2.54-2.44 (m, 1H, H5′ under DMSO peak), 2.18-2.07 (m, 1H, H5′). HPLC: t_{R}=6.948 min (Method B). UV-purity at 210 nm=>95%.

### (1R,2S,3R,5S)-3-(4-amino-7H-pyrrolo[2,3-a]pyrimidin-7-yl)-5-(thiophen-2-yl) cyclopentane-1,2-diol (390)

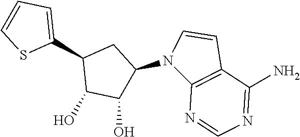 [SMILES: Nc1ncnc2c1ccn2[C@@H]1C[C@H](c2cccs2)[C@@H](O)[C@H]1O]

Following the general procedure for deprotection Q, compound 390 was obtained starting from compound 271 (0.02 g, 0.05 mmol) in a mixture of water and TFA (0.03 M, 1.60 mL, 1:4) after preparative HPLC using method D (water/MeCN; 0.05% TFA) as white foam (10 mg, 55.6%). 1H NMR (400 MHZ, DMSO-d₆) δ8.39 (s, 1H, H2), 7.71 (d, J=3.7 Hz, 1H, H6), 7.38 (dd, J=4.9, 1.4 Hz, 1H, H5 thiophene), 7.03-6.98 (m, 2H, H3, H4 thiophene), 6.96 (d, J=3.6 Hz, 1H, H5), 4.99 (dt, J=10.9, 7.3 Hz, 1H, H1′), 4.26 (t, J=6.7 Hz, 1H, H2′), 4.03 (t, J=6.5 Hz, 1H, H3′), 3.35 (dt, J=11.0, 7.0 Hz, 1H, H4′), 2.59-2.51 (m, 1H, H5′), 2.05 (dt, J=12.4, 11.0 Hz, 1H, H5′). HPLC: t_{R}=12.673 min (Method A). UV-purity at 210 nm=96.0%.

### (1R,2S,3R,5R)-3-(4-amino-7H-pyrrolo[2,3-d]pyrimidin-7-yl)-5-(1H-pyrazol-5-yl) cyclopentane-1,2-diol (391)

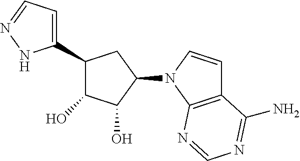 [SMILES: Nc1ncnc2c1ccn2[C@@H]1C[C@H](c2ccn[nH]2)[C@@H](O)[C@H]1O]

Following the general procedure for deprotection Q, compound 391 was obtained starting from compound 272 (0.03 g, 0.07 mmol) in a mixture of water and TFA (0.05 M, 1.50 mL, 1:4) after preparative HPLC using method E (water/MeCN; 0.05% TFA) as white foam (17.30 mg, 79%). 1H NMR (400 MHZ, DMSO-d₆) δ 8.39 (s, 1H, H2), 7.87 (dd, J=2.3, 0.7 Hz, 1H, H3 pyrazol), 7.83 (d, J=3.6 Hz, 1H, H6), 7.56 (d, J=1.7 Hz, 1H, —NH pyrazol), 7.00 (d, J=3.6 Hz, 1H, H5), 6.29 (dd, J=1.9 Hz, 1H, H4 pyrazol), 5.12-5.04 (m, 1H, H1′), 4.68 (td, J=8.2, 4.5 Hz, 1H, H4′), 4.42 (dd, J=7.2, 5.8 Hz, 1H, H2′), 4.13-4.09 (m, 1H, H3′), 2.75 (dt, J=13.6, 8.7 Hz, 1H, H5′), 2.39-2.31 (m, 1H, H5′). APCI calcd.: 300.13; found: 300.90 [M+H]⁺. HPLC: [R=8.818 min (Method B). UV-purity at 210 nm=100%.

### (1R,2S,3R,5R)-3-(4-amino-2-chloro-7H-pyrrolo[2,3-d]pyrimidin-7-yl)-5-(quinolin-6-yl) cyclopentane-1,2-diol (392)

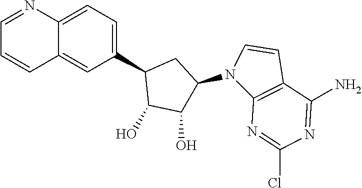 [SMILES: Nc1nc(Cl)nc2c1ccn2[C@@H]1C[C@H](c2ccc3ncccc3c2)[C@@H](O)[C@H]1O]

Following the general procedure for deprotection Q, compound 392 was obtained starting from compound 273 (0.07 g, 0.15 mmol) in a mixture of water and TFA (0.05 M, 2.00 mL, 1:4) after preparative HPLC using method D (water/MeCN; 0.05% TFA) as white foam (28 mg, 69%). 1H NMR (400 MHZ, DMSO-d₆) δ9.11 (dd, J=4.8, 1.5 Hz, 1H, H2 quinoline), 8.81 (d, J=9.5 Hz, 1H, H4, quinoline), 8.19-8.11 (m, 3H, H5, H6, H7 quinoline), 7.86 (dd, J=8.3, 4.9 Hz, 1H, H3 quinoline), 7.50 (bs, 2H, —CN H₂), 7.43 (d, J=3.6 Hz, 1H, H6), 6.62 (d, J=3.5 Hz, 1H, H5), 4.90 (dt, J=11.0, 7.5 Hz, 1H, H1′), 4.31 (t, J=6.5 Hz, 1H, H2′), 4.18 (t, J=6.7 Hz, 1H, H3′), 3.38 (dt, J=11.1, 7.2 Hz, 1H, H4′), 2.53-2.43 (m, 1H, H5′ under DMSO peak), 2.19-2.09 (m, 1H, H5′). APCI calc.: 395.11; found: 395.60/397.50.60 [M+H]⁺. HPLC: t_{R}=10.879 min (Method A). UV-purity at 210 nm=100%.

### (1R,2S,3R,5R)-3-(4-amino-5-bromo-2-chloro-7H-pyrrolo[2,3-d]pyrimidin-7-yl)-5-(3-(aminomethyl)phen-yl) cyclopentane-1,2-diol (393)

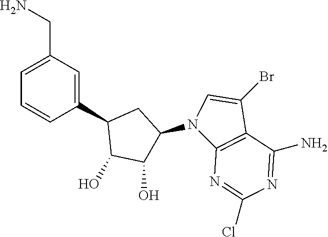 [SMILES: NCc1cccc([C@H]2C[C@@H](n3cc(Br)c4c(N)nc(Cl)nc43)[C@H](O)[C@@H]2O)c1]

Following the general procedure for deprotection Q, compound 393 was obtained starting from compound 274 (0.21 g, 0.35 mmol) in a mixture of water and TFA (0.10 M, 3.50 mL, 1:4). 36 mg of the crude product were purified over preparative HPLC using method D (water/MeCN; 0.05% TFA) as white foam (13.30 mg, 0.03 mmol). ¹H NMR (400 MHZ, DMSO-d₆) δ 8.21-8.11 (m, 3H, —CH₂NH₃+), 7.73 (s, 1H, H6, adenine), 7.49-7.44 (m, 2H, H4, H5 aryl), 7.41 (t, J=7.5 Hz, 1H, H2 aryl), 7.35-7.30 (m, 1H, H6 aryl), 4.90 (dt, J=11.5, 7.5 Hz, 1H, H1′), 4.33-4.27 (m, 1H, H2′), 4.08-4.02 (m, 2H, —CH₂NH₃+), 3.98 (dd, J=8.3, 4.0 Hz, 1H, H3′), 3.10 (ddd, J=11.4, 7.5, 6.0 Hz, 1H, H4′), 2.41-2.32 (m, 1H, H5′), 1.96 (q, J=11.5 Hz, 1H, H5′). HPLC: t_{R}=9.987 min (Method A-XBridge). UV-purity at 210 nm=95.5%.

### (1R,2S,3R,5R)-3-(4-amino-5-(thiazol-2-yl)-7H-pyrrolo[2,3-d]pyrimidin-7-yl)-5-(3,5-dichlorophenyl) cyclo-pentane-1,2-diol (394)

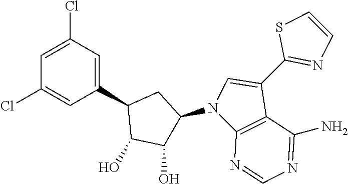 [SMILES: Nc1ncnc2c1c(-c1nccs1)cn2[C@@H]1C[C@H](c2cc(Cl)cc(Cl)c2)[C@@H](O)[C@H]1O]

Following the general procedure for deprotection Q, compound 394 was obtained starting from compound 283 (0.06 g, 0.13 mmol) in a mixture of water and TFA (0.05 M, 2.50 mL, 1:4) after preparative HPLC using method D (water/MeCN; 0.05% TFA) as white foam (13.30 mg, 0.03 mmol). ¹H NMR (400 MHZ, DMSO-d₆) δ 8.62 (s, 1H, H2), 8.39 (s, 1H, H6), 7.90 (d, J=3.4 Hz, 1H, H4 thiazole), 7.77 (d, J=3.4 Hz, 1H, H5 thiazole), 7.52-7.49 (m, 3H, olp-Ar), 5.05 (dt, J=11.4, 7.0 Hz, 1H, H1′), 4.32 (t, J=6.9 Hz, 1H, H2′), 4.08 (t, J=7.1 Hz, 1H, H3′), 3.15 (dt, J=12.3, 7.0 Hz, 1H, H4′), 2.37 (dt, J=12.3, 7.2 Hz, 1H, H5′), 2.13 (q, J=12.0 Hz, 1H, H5′). APCI calc.: 461.05; found: 462.20/464.20 [M+H]⁺. HPLC: [R=18.268 min (Method A). UV-purity at 210 nm=98.9%.

### (1R,2S,3R,5R)-3-(4-amino-5-(thiazol-2-yl)-7H-pyrrolo[2,3-d]pyrimidin-7-yl)-5-phenylcyclopentane-1,2-diol (395)

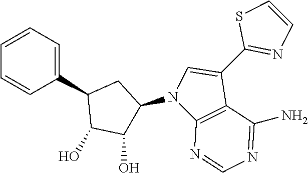 [SMILES: Nc1ncnc2c1c(-c1nccs1)cn2[C@@H]1C[C@H](c2ccccc2)[C@@H](O)[C@H]1O]

Following the general procedure for deprotection Q, compound 395 was obtained starting from compound 284 (0.02 g, 0.05 mmol) in a mixture of water and TFA (0.03 M, 1.60 mL, 1:4) after preparative HPLC using method D (water/MeCN; 0.05% TFA) as white foam (17.30 mg, 96%). 1H NMR (400 MHZ, DMSO-d₆) δ8.68 (s, 1H, H2), 8.43 (s, 1H, H6), 7.90 (d, J=3.4 Hz, 1H, H4 thiazole), 7.78 (d, J=3.4 Hz, 1H, H5 thiazole), 7.45-7.40 (m, 2H, o-Ar), 7.35 (dd, J=8.4, 6.9 Hz, 2H, m-Ar), 7.29-7.21 (m, 1H, p-Ar), 5.07 (dt, J=11.6, 7.3 Hz, 1H, H1′), 4.40 (t, J=7.2 Hz, 1H, H2′), 4.07 (t, J=6.6 Hz, 1H, H3′), 3.11 (dt, J=12.8, 6.9 Hz, 1H, H4′), 2.42-2.33 (m, 1H, H5′), 2.12 (q, J=11.9 Hz, 1H, H5′). HPLC: t_{R}=15.374 min (Method A). UV-purity at 210 nm=100.0%.

### (1R,2S,3R,5R)-3-(4-amino-5-ethyl-7H-pyrrolo[2,3-d]pyrimidin-7-yl)-5-phenylcyclopentane-1,2-diol (396)

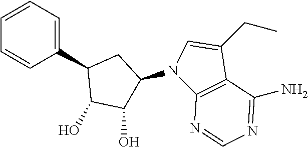 [SMILES: CCc1cn([C@@H]2C[C@H](c3ccccc3)[C@@H](O)[C@H]2O)c2ncnc(N)c12]

Following the general procedure for deprotection Q, compound 396 was obtained starting from crude compound 286 in a mixture of water and TFA (0.01 M, 4.50 mL, 1:4) after preparative HPLC using method D (water/MeCN; 0.05% TFA) as white foam (13.6 mg, 90%). ¹H NMR (400 MHZ, DMSO-d₆) δ 8.35 (s, 1H, H2), 8.28 (s, 2H, —CN H₂), 7.60 (d, J=1.2 Hz, 1H, H6), 7.42-7.37 (m, 2H, o-Ar), 7.34 (dd, J=8.4, 6.8 Hz, 2H, m-Ar), 7.27-7.20 (m, 1H, p-Ar), 4.98 (dt, J=11.4, 7.3 Hz, 1H, H1′), 4.27 (t, J=7.0 Hz, 1H, H2′), 4.03 (t, J=6.7 Hz, 1H, H3′), 3.09 (dt, J=11.8, 7.0 Hz, 1H, H4′), 2.85 (qd, J=7.4, 1.1 Hz, 2H, —CH₂CH₃), 2.32 (dt, J=12.4, 7.3 Hz, 1H, H5′), 2.01 (q, J=11.9 Hz, 1H, H5′), 1.25 (t, J=7.4 Hz, 3H, —CH₂CH₃). HPLC: t_{R}=14.398 min (Method A). UV-purity at 210 nm=100%.

### (1R,2S,3R,5R)-3-(4-amino-7H-pyrrolo[2,3-d]pyrimidin-7-yl)-5-(piperidin-4-yl) cyclopentane-1,2-diol (397)

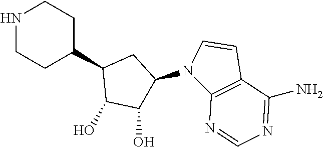 [SMILES: Nc1ncnc2c1ccn2[C@@H]1C[C@H](C2CCNCC2)[C@@H](O)[C@H]1O]

Following the general procedure for deprotection Q, compound 397 was obtained starting from compound 194 (0.05 g, 0.11 mmol) in a mixture of water and TFA (0.10 M, 1.10 mL, 1:4) after preparative HPLC using method E (water/MeCN; 0.05% TFA) as white foam (22.32 mg, 48%, 1 TFA salt,). ¹H NMR (400 MHZ, DMSO) δ8.58-8.50 (m, 1H, —NH₂+), 8.36 (s, 1H, H2), 8.32-8.21 (m, 1H, —NH₂+), 7.65 (d, J=3.7 Hz, 1H, H6), 6.94 (d, J=3.7 Hz, 1H, H5), 4.85 (dt, J=11.5, 8.0 Hz, 1H, H1′), 4.13 (dd, J=8.8, 6.4 Hz, 1H, H2′), 3.80 (dd, J=6.5, 4.3 Hz, 1H, H3′), 3.36-3.24 (m, 2H, —N (CH₂) 2 (CH₂) 2), 2.93-2.76 (m, 2H, —N(CH₂) 2 (CH₂) 2), 2.16-1.99 (m, 2H, H5′, —N(CH₂) 2 (CH₂) 2CH—), 1.81-1.66 (m, 2H, H4′, —N(CH₂) 2 (CH₂) 2CH—), 1.66-1.51 (m, 1H, H5, —N(CH₂) 2 (CH₂) 2CH—), 1.47-1.28 (m, 2H, —N(CH₂) 2 (CH₂) 2CH—). APCI calc. for C₁₆H₂₄N₅O₂. HPLC: t_{R}=6.633 min (Method B). UV-purity at 210 nm=96.4%.

### (1R,2S,3R,5R)-3-(4-amino-7H-pyrrolo[2,3-d]pyrimidin-7-yl)-5-((R)-pyrrolidin-2-yl) cyclopentane-1,2-diol (398) & (1R,2S,3R,5R)-3-(4-amino-7H-pyrrolo[2,3-a]pyrimidin-7-yl)-5-((S)-pyrrolidin-2-yl) cyclopentane-1,2-diol (399)

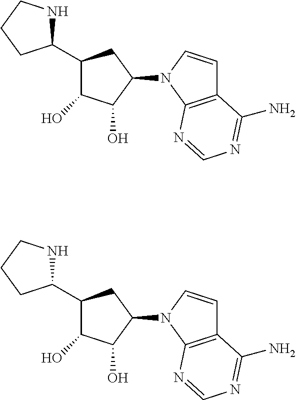 [SMILES: Nc1ncnc2c1ccn2[C@@H]1C[C@H]([C@H]2CCCN2)[C@@H](O)[C@H]1O ; Nc1ncnc2c1ccn2[C@@H]1C[C@H]([C@@H]2CCCN2)[C@@H](O)[C@H]1O]

Following the general procedure for deprotection Q, compounds 398 & 399 were obtained starting from compound 275 (0.09 g, 0.20 mmol) in a mixture of water and TFA (0.05 M, 4.00 mL, 1:4) after preparative HPLC using method E (water/MeCN; 0.05% TFA) as white foams (1 TFA salt,).

¹H NMR (400 MHZ, DMSO) δ 9.33-9.19 (m, 1H, NH₂+), 8.58-8.47 (m, 1H, NH₂+), 8.36 (s, 1H, H2), 7.62 (d, J=3.5 Hz, 1H, H6), 6.94 (d, J=3.5 Hz, 1H, H5), 4.91 (dt, J=10.8, 7.9 Hz, 1H, H1′), 4.20 (dd, J=8.2, 6.1 Hz, 1H, H2′), 3.88-3.83 (m, 1H, H3′), 3.57-3.44 (m, 1H, —NH₂+CH₂CH₂CH—), 3.26-3.09 (m, 2H, —NH₂+CH₂CH₂CH₂CH—), 2.35-2.20 (m, 2H, H5′, —NH₂+CH₂CH₂CH₂—), 2.20-2.10 (m, 1H, H4′), 2.04-1.81 (m, 2H, —NH₂+CH₂CH₂CH₂—), 1.80-1.63 (m, 2H, H5′, —NH₂+CH₂CH₂CH₂—). HPLC: t_{R}=6.429 min (Method B). UV-purity at 210 nm=100%.

¹H NMR (400 MHZ, DMSO) δ9.28-9.18 (m, 1H, —NH₂+), 8.49-8.40 (m, 1H, —NH₂+), 8.38 (s, 1H, H2), 7.67 (d, J=3.6 Hz, 1H, H6), 6.96 (d, J=3.6 Hz, 1H, H5), 4.94 (dt, J=10.9, 7.5 Hz, 1H, H1′), 4.18 (dd, J=7.5, 6.0 Hz, 1H, H2′), 3.98 (t,)=5.5 Hz, 1H, H3′), 3.56-3.43 (m, 1H, —NH₂+CH₂CH₂CH—), 3.28-3.12 (m, 2H, —NH₂+CH₂CH₂CH₂CH—), 2.30-2.14 (m, 2H, H4′, H5′), 2.12-2.01 (m, 1H, —NH₂+CH₂CH₂CH₂—), 1.99-1.78 (m, 1H, —NH₂+CH₂CH₂CH₂CH—), 1.70-1.50 (m, 2H, H5′, —NH₂+CH₂CH₂CH—). HPLC: {R=7.316 min (Method B). UV-purity at 210 nm=95.0%.

### (1R,2S,3R,5R)-3-(4-amino-7H-pyrrolo[2,3-d]pyrimidin-7-yl)-5-((R)-piperidin-2-yl) cyclopentane-1,2-diol (400)

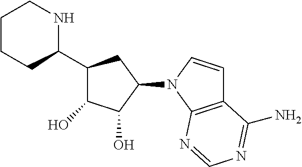 [SMILES: Nc1ncnc2c1ccn2[C@@H]1C[C@H]([C@H]2CCCCN2)[C@@H](O)[C@H]1O]

Following the general procedure for deprotection Q, compound 400 were obtained starting from compound 276 (0.04 g, 0.09 mmol) in a mixture of water and TFA (0.05 M, 1.90 mL, 1:4) after preparative HPLC using method E (water/MeCN; 0.05% TFA) as white foam (34.4 mg, 86%, 1 TFA salt). ¹H NMR (400 MHZ, DMSO-d₆) δ8.49-8.40 (m, 2H, —NH₂+), 8.39 (s, 1H, H2), 7.63 (d, J=3.6 Hz, 1H, H6), 6.96 (d, J=3.6 Hz, 1H, H5), 4.94-4.84 (m, 1H, H1′), 4.13 (dd, J=8.4, 6.4 Hz, 1H, H2′), 3.93 (dd, J=6.2, 4.6 Hz, 1H, H3′), 3.26 (d, J=12.5 Hz, 1H, —NH₂+CH₂CH₂CH₂CH₂CH—), 3.19-3.08 (m, 1H, —NH₂+CH₂CH₂CH₂CH₂CH—), 2.94-2.80 (m, 1H, —NH₂+CH₂CH₂CH₂CH—), 2.24-2.05 (m, 3H, H5′, H4′, —NH₂+CH₂CH₂CH₂CH₂CH—), 1.85-1.70 (m, 3H, H5′, —NH₂+CH₂CH₂CH₂CH₂CH—), 1.67-1.52 (m, 1H, NH₂+CH₂CH₂CH₂CH₂CH—), 1.52-1.37 (m, 2H, NH₂+CH₂CH₂CH₂CH₂CH—). HPLC: t_{R}=5.544 min (Method B). UV-purity at 210 nm=98.8%.

### (1S,2R,3R,5R)-3-((S)-piperidin-3-yl)-5-(5-(thiazol-2-yl)-7//pyrrolo[2,3-a]pyrimidin-7-yl) cyclopentane-1,2-diol (401) & (1S,2R,3R,5R)-3-((R)-piperidin-3-yl)-5-(5-(thiazol-2-yl)-7H-pyrrolo[2,3-a]pyrimidin-7-yl) cyclopentane-1,2-diol (402)

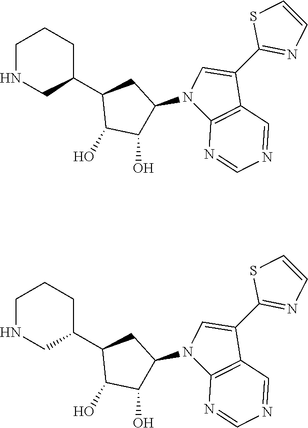 [SMILES: O[C@@H]1[C@H](O)[C@@H]([C@@H]2CCCNC2)C[C@H]1n1cc(-c2nccs2)c2cncnc21 ; O[C@@H]1[C@H](O)[C@@H]([C@H]2CCCNC2)C[C@H]1n1cc(-c2nccs2)c2cncnc21]

Following the general procedure for deprotection Q, compounds 401 & 402 were obtained starting from compound 258 (0.14 g, 0.27 mmol) in a mixture of water and TFA (0.05 M, 5.40 mL, 1:4) after preparative HPLC using method E (water/MeCN; 0.05% TFA) as white foams (134.5 mg, 100%, 1 TFA salt).

¹H NMR (400 MHZ, DMSO) δ9.54 (s, 1H, H2), 8.97 (s, 1H, H4), 8.70-8.62 (m, 2H, H6, —N/2+), 8.39-8.26 (m, 1H, —NH₂+), 7.93 (d, J=3.3 Hz, 1H, H4 thiazole), 7.73 (d, J=3.3 Hz, 1H, H5 thiazole), 5.05-4.95 (m, 1H, H1′), 4.32 (dd, J=8.9, 6.7 Hz, 1H, H2′), 3.90 (dd, J=6.7, 4.1 Hz, 1H, H3′), 3.27-3.17 (m, 2H, —NH₂+(CH₂) 2—), 2.89-2.77 (m, 1H, —NH₂+(CH₂) 2—), 2.76-2.65 (m, 1H, —NH₂+(CH₂) 2—), 2.15 (dt, J=11.7, 6.8 Hz, 1H, H5′), 2.07-1.98 (m, 1H, —NH₂+CH₂CH—), 1.90-1.67 (m, 4H, H4′, H5′, —NH₂+CH₂CH₂CH₂—), 1.67-1.54 (m, 1H, —NH₂+CH₂CH₂CH₂—), 1.38-1.25 (m, 1H, —NH₂+CH₂CH₂CH₂—). HPLC: t_{R}=8.120 min (Method B). UV-purity at 210 nm=100.0%.

¹H NMR (400 MHZ, DMSO) δ9.54 (s, 1H, H2), 8.98 (s, 1H, H4), 8.68 (s, 1H, H6), 8.67-8.61 (m, 1H, —NH₂+), 8.41-8.28 (m, 1H, —N/2+), 7.93 (d, J=3.3 Hz, 1H, H4 thiazole), 7.73 (d, J=3.3 Hz, 1H, H5 thiazole), 5.05-4.96 (m, 1H, H1′), 4.27 (dd, J=8.4, 6.7 Hz, 1H, H2′), 3.85 (dd, J=6.7, 4.5 Hz, 1H, H3′), 3.49 (d, J=12.2 Hz, 1H, —NH₂+(CH₂) 2—), 3.25 (d, J=12.4 Hz, 1H, —NH₂+(CH₂) 2—), 2.87-2.75 (m, 1H, —NH₂+(CH₂) 2—), 2.72-2.60 (m, 1H, —NH₂+(CH₂) 2—), 2.18-2.09 (m, 1H, H5′), 1.86-1.70 (m, 5H, H4′, H5′, —NH₂+CH₂CH—, —NH₂+CH₂CH₂CH₂—), 1.66-1.51 (m, 1H, —NH₂+CH₂CH₂CH₂—), 1.28-1.15 (m, 1H, —NH₂+CH₂CH₂CH₂—). HPLC: t_{R}=8.756 min (Method B). UV-purity at 210 nm=100.0%.

### (1R,2S,3R,5R)-3-(4-(difluoromethyl)-7H-pyrrolo[2,3-d]pyrimidin-7-yl)-5-((R)-piperidin-3-yl) cyclopentane-1,2-diol (403)

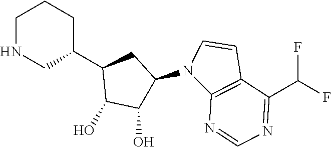 [SMILES: O[C@@H]1[C@H](O)[C@@H]([C@H]2CCCNC2)C[C@H]1n1ccc2c(C(F)F)ncnc21]

Following the general procedure for deprotection Q, compound 403 were obtained starting from compound 259 (0.19 g, 0.37 mmol) in a mixture of water and TFA (0.05 M, 7.50 mL, 1:4) after preparative HPLC using method E (water/MeCN; 0.05% TFA) as white foams (102.52 mg, 58.9%, 1 TFA salt). ¹H NMR (400 MHZ, DMSO) δ 8.89 (s, 1H, H2), 8.70-8.60 (m, 1H, —NH₂+), 8.37-8.25 (m, 1H, —NH₂+), 7.97 (d, J=3.7 Hz, 1H, H6), 7.36-7.06 (m, 1H, —CHF₂), 6.82-6.78 (m, 1H, H5), 5.02-4.90 (m, 1H, H1′), 4.24 (dd, J=8.7, 6.6 Hz, 1H, H2′), 3.88 (dd, J=6.5, 4.3 Hz, 1H, H3′), 3.26-3.16 (m, 2H, —NH₂+(CH₂) 2—), 2.89-2.75 (m, 1H, —NH₂+(CH₂) 2—), 2.74-2.62 (m, 1H, —NH₂+(CH₂) 2—), 2.13 (dt, J=11.9, 7.2 Hz, 1H, H5′), 2.01 (d, J=13.2 Hz, 1H, —NH₂+CH₂CH—), 1.88-1.53 (m, 5H, H4′, H5′, —NH₂+CH₂CH₂CH₂—, NH₂+CH₂CH₂CH₂—), 1.38-1.22 (m, 1H, NH₂+CH₂CH₂CH₂—). HPLC: t_{R}=7.679 min (Method B). UV-purity at 210 nm=96.3%.

### (1R,2S,3R,5R)-3-(4-amino-7H-pyrrolo[2,3-d]pyrimidin-7-yl)-5-(3-hydroxyphenyl) cyclopentane-1,2-diol (404)

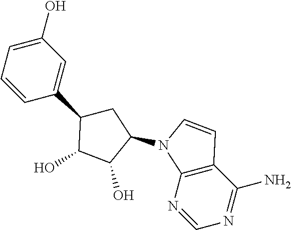 [SMILES: Nc1ncnc2c1ccn2[C@@H]1C[C@H](c2cccc(O)c2)[C@@H](O)[C@H]1O]

Following the general procedure for deprotection Q, compound 404 was obtained starting from compound 277 (0.06 g, 0.16 mmol) in a mixture of water and TFA (6.80 mL, 1:4) after preparative HPLC using method D (water/MeCN; 0.05% TFA) as white foam (19.60 mg, 37%). APCI: calcd. for C₁₇H₁₈N₄O₃ [M+H]⁺: 327.1 found 326.8. t_{R}=10.96 min, UV-purity at 210 nm=98%. ¹H NMR (400 MHZ, DMSO-d₆) δ 9.32 (bs, 1H, phenyl OH), 8.96-8.66 (br d, 2H, NH₂), 8.39 (s, 1H, H2), 7.77 (d, /=3.6 Hz, 1H, H8), 7.12 (t, J=7.8 Hz, 1H, m-H), 6.96 (d, J=3.6 Hz, 1H, H7), 6.82-6.78 (m, 2H, 2 x o-H), 6.63 (ddd, J=8.1 Hz, 2.3 Hz, 1.0 Hz, 1H, p-H), 4.97 (dt, J=11.3 Hz, 7.3 Hz, 1H, H1′), 4.29 (t, J=7.0 Hz, 1H, H2′), 4.02 (t, J=7.0 Hz, 1H, H3′), 3.02 (dt, J=11.4 Hz, 7.0 Hz, 1H, H4′), 2.35 (dt, J=12.6 Hz, 7.3 Hz, 1H, H5′A), 2.00 (q, J=11.8, 1H, H5′B)

### (1R,2S,3R,5R)-3-(4-amino-7H-pyrrolo[2,3-d]pyrimidin-7-yl)-5-(4-hydroxyphenyl) cyclopentane-1,2-diol (405)

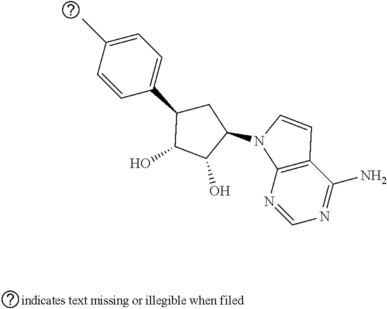 [SMILES: Cc1ccc([C@H]2C[C@@H](n3ccc4c(N)ncnc43)[C@H](O)[C@@H]2O)cc1]

Following the general procedure for deprotection Q, compound 405 was obtained starting from compound 278 (0.03 g, 0.07 mmol) in a mixture of water and TFA (2.80 mL, 1:4) after preparative HPLC using method D (water/MeCN; 0.05% TFA) as white foam (15.01 mg, 71%). APCI: calcd. for C₁₇H₁₈ N₄O₃ [M+H]⁺: 327.1 found 326.9. HPLC: t_{R}=9.98 min, UV-purity at 210 nm=99%. ¹H NMR (400 MHZ, DMSO-d₆) δ9.36-8.46 (s, 1H, NH₂, phenyl OH), 8.39 (s, 1H, H2), 7.78 (d, J=3.6 Hz, 1H, H8), 7.19-7.14 (m, 2H, o-H), 6.96 (d, J=3.6 Hz, 1H, H7), 6.74-6.60 (m, 2H, m-H), 4.97 (dt, J=11.2 Hz, 7.4 Hz, 1H, H1′), 4.27 (t, J=7.0 Hz, 1H, H2′), 3.98-3.93 (m, 1H, H3′)3.00 (dt, J=11.4 Hz, 7.0 Hz, 1H, H4′), 2.37-2.28 (m, H5′A), 1.95 (q, J=11.8 Hz, 1H, H5′B).

### (1S,2R,3R,5R)-3-(4-hydroxyphenyl)-5-(4-(methylamino)-7H-pyrrolo[2,3-a]pyrimidin-7-yl) cyclopentane-1,2-diol (406)

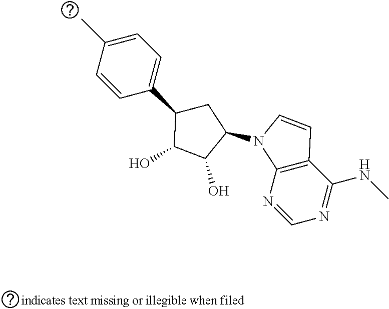

Following the general procedure for deprotection Q, compound 406 was obtained starting from compound 279 (0.05 g) in a mixture of water and TFA (3.20 mL, 1:4) after preparative HPLC using method D (water/MeCN; 0.05% TFA) as a yellowish foam (19.70 mg, 20%). APCI: calcd. for [M+H]⁺: 341.2 found 340.9. HPLC: t_{R}=10.57 min, UV-purity at 210 nm=98%. ¹H NMR (400 MHZ, DMSO-d₆) δ9.50 (br s, 1H, —NH—CH₃), 9.25 (br s, 1H, —phenyl OH), 8.40 (s, 1H, H2), 7.75 (s, 1H, H8), 7.20-7.15 (m, 2H, 2 x o-H), 6.92 (s, 1H, H7), 6.75-6.71 (m, 2H, m-H), 5.01-4.91 (m, 1H, H1′), 4.28 (t, J=6.9 Hz, 1H, H2′), 4.01-3.94 (m, 1H, H3′), 3.10 (s, 3H, —NH—CH₃), 3.00 (dt, J=11.7, 7.0 Hz, 1H, H4′), 2.38-2.28 (m, 1H, H5′A), 1.96 (q, J=11.7 Hz, 1H, H5′B).

### (1R,2S,3R,5R)-3-(4-amino-7H-pyrrolo[2,3-d]pyrimidin-7-yl)-5-(3-chlorophenyl) cyclopentane-1,2-diol (407)

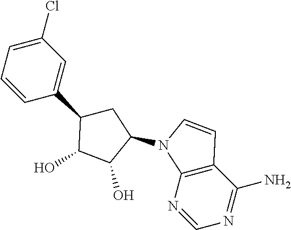 [SMILES: Nc1ncnc2c1ccn2[C@@H]1C[C@H](c2cccc(Cl)c2)[C@@H](O)[C@H]1O]

Following the general procedure for deprotection Q, compound 407 was obtained starting from compound 280 (0.02 g, 0.06 mmol) in a mixture of water and TFA (1.90 mL, 1:4) after preparative HPLC using method D (water/MeCN; 0.05% TFA) as a white foam (13.00 mg, 65%). APCI: calcd. for C₁₇H₁₇ClN₄O₂ [M+H]⁺: 345.1 found 344.8. HPLC: t_{R}=14.1 min, UV-purity at 210 nm=99%. 1H NMR (400 MHZ, DMSO-d₆) δ 9.39-8.44 (br m, 2H, NH₂), 8.39 (s, 1H, H2), 7.82 (d, J=3.6 Hz, 1H, H8), 7.50-7.49 (m 1H, m-H), 7.41-7.35 (m, 2H, o-H, p-H), 7.34-7.29 (m, 1H, o-H), 6.97 (d, J=3.7 Hz, 1H, H7), 5.00 (dt, J=11.2, 7.2 Hz, 1H, H1′), 4.26 (t, J=6.7 Hz, 1H, H2′), 4.07 (t, J=6.9 Hz, 1H, H3′), 3.14 (dt, J=11.8 Hz, 7.0 Hz, 1H, H4′), 2.39 (dt, J=12.3, 7.3 Hz, 1H, H5′A), 2.04 (q, J=11.9 Hz, 1H, H5′B).

### 1-(3-((1R,2R,3S,4R)-4-(4-amino-7H-pyrrolo[2,3-d]pyrimidin-7-yl)-2,3-dihydroxycyclopentyl)phenyl)ethan-1-one (408)

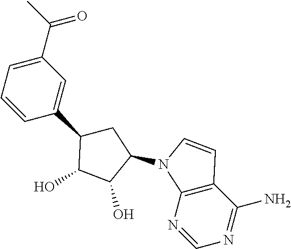 [SMILES: CC(=O)c1cccc([C@H]2C[C@@H](n3ccc4c(N)ncnc43)[C@H](O)[C@@H]2O)c1]

Following the general procedure for deprotection Q, compound 408 was obtained starting from compound 281 (0.04 g, 0.08 mmol) in a mixture of water and TFA (2.60 mL, 1:4) after preparative HPLC using method D (water/MeCN; 0.05% TFA) as a white foam (21.00 mg, 75%). APCI: calcd. for C₁₉H₂₀N₄O₃ [M+H]⁺: 353.2 found. 352.8 HPLC(Jung40.): t_{R}=12.37 min, UV-purity at 210 nm=98%. ¹H NMR (400 MHZ, DMSO-d₆) δ 9.33-8.44 (br d, 2H, —NH₂), 8.40 (s, 1H, H2), 7.96-7.95 (m, 1H, o-H), 7.85 (dt, J=7.7, 1.4 Hz, 1H, p-H), 7.81 (d, J=3.7 Hz, 1H, H8), 7.68 (dt, J=7.8, 1H, o-H), 7.51 (t, J=7.7 Hz, 1H, m-H), 6.97 (d, J=3.6 Hz, 1H, H7), 5.01 (dt, J=11.2, 7.1 Hz, 1H, H1′), 4.30 (t, J=6.8 Hz, 1H, H2′), 4.10 (t, J=6.9 Hz, 1H, H3′), 3.21 (dt, J=11.8, 7.1 Hz, 1H, H4′), 2.60 (s, CH₃, acetyl), 2.46-2.37 (m,,1H, H5′A), 2.08 (q, J=11.9 Hz, 1H, H5′B).

### (1r,2s,3r,5r)-3-(4-amino-7H-pyrrolo[2,3-d]pyrimidin-7-yl)-5-(4-aminophenyl) cyclopentane-1,2-diol (409)

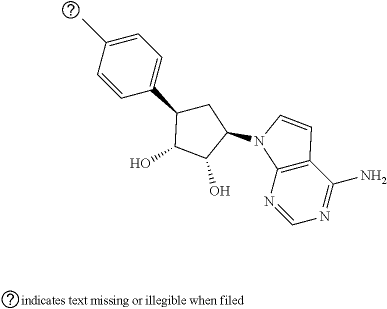 [SMILES: Cc1ccc([C@H]2C[C@@H](n3ccc4c(N)ncnc43)[C@H](O)[C@@H]2O)cc1]

260 mg (0.56 mmol; 1.00 eq) of tert-butyl (4-((3ar,4r,6as)-6-hydroxy-2,2-dimethyltetrahydro-4H cyclopenta[d][1,3]dioxol-4-yl)phenyl)carbamate were dissolved in 16 mL TFA/H₂O (1:1) and stirred at RT overnight. The solvent was removed under reduced pressure and the resulting residue was purified by HPLC according to method D (MeCN/H₂O+0.1% TFA; 0/100% to 5/95%; TR=7.22 min; UV-purity at 210, 230, 254, 242 and 275 nm >98%). 33 mg (0.10 mmol; 18%) of the title compound were obtained in the form of a colorless foam. APCI-MS (+) m/z for C₁₇H₁₉N₅O₂: calc.: 325.37; found: 325.9 and 327.0. ¹H-NMR: (400 MHZ, DMSO-d₆)}=9.14 (bs, 1H), 8.69 (bs, 1H), 8.41 (s, 1H), 7.80 (d, J=3.6 Hz, 1H), 7.41-7.34 (m, 2H), 7.13-7.06 (m, 2H), 6.99 (d, J=3.5 Hz, 1H), 5.04-4.97 (m, 1H), 4.29 (t, J=6.8 Hz, 1H), 4.02 (t, J=6.7 Hz, 1H), 3.13-3.07 (m, 1H), 2.42-2.34 (m, 1H), 2.04-1.94 (m, 1H) ppm.

### (1R,2S,3R,5R)-3-(4-amino-7H-pyrrolo[2,3-d]pyrimidin-7-yl)-5-(3-aminophenyl) cyclopentane-1,2-diol (410)

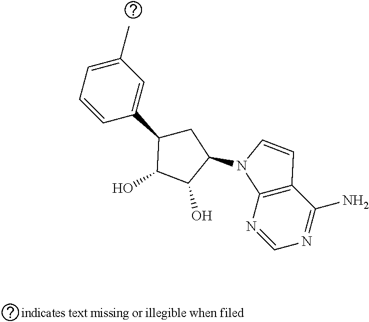 [SMILES: Cc1cccc([C@H]2C[C@@H](n3ccc4c(N)ncnc43)[C@H](O)[C@@H]2O)c1]

170 mg (0.36 mmol; 1.00 eq) of tert-butyl (3-((3ar,4r,6r,6as)-6-(4-amino-7H-pyrrolo[2,3-d]pyrimidin-7-yl)-2,2-dimethyltetrahydro-4H-cyclopenta[d][1,3]dioxol-4-yl)phenyl)carbamate were dissolved in 16 mL TFA/H₂O (1:1) and stirred at RT overnight. The solvent was removed under reduced pressure and the resulting residue was purified by HPLC according to method D (MeCN/H₂O+0.1% TFA; 0/100% to 5/95%; TR=7.86 min; UV-purity at 210, 230, 254, 242 and 275 nm >99%). 118 mg (0.37 mmol; quantitative) of the title compound were obtained in the form of a colorless foam. APCI-MS (+) m/z for C₁₇H₁₉N₅O₂: calc.: 325.37; found: 325.8 and 326.8. ¹H-NMR: (400 MHZ, DMSO-d₆)}=9.16 (bs, 1H), 8.73 (bs, 1H), 8.41 (s, 1H), 7.78 (d, J=3.6 Hz, 1H), 7.30-7.26 (m, 1H), 7.13-7.03 (m, 2H), 6.98 (d, J=3.6 Hz, 1H), 6.94-6.88 (m, 1H), 5.03-4.97 (m, 1H), 4.30 (t, J=6.9 Hz, 1H), 4.03 (t, J=6.6 Hz, 1H), 3.17-3.05 (m, 1H), 2.43-2.36 (m, 1H), 2.06-1.97 (m, 1H) ppm.

### Preparation of (2R,3S,4R,5R)-2-(aminomethyl)-5-(6,7,8,9-tetrahydro-2H-2,3,5,6-tetraazabenzo[cd]azulen-2-yl) tetrahydrofuran-3,4-diol 5-(3-((tert-butyldimethylsilyl)oxy)propyl)-4-chloro-7H-pyrrolo[2,3-d]pyrimidine (411)

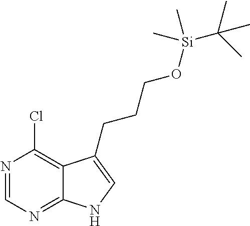 [SMILES: CC(C)(C)[Si](C)(C)OCCCc1c[nH]c2ncnc(Cl)c12]

A heat-dried three-necked round bottom flask was equipped with a magnetic stirring bar and the thermometer. Then, the flask was charged with 5-bromo-4-chloro-7H-pyrrolo[2,3-d]pyrimidine (2.80 g,11.92 mmol) under nitrogen atmosphere. The solid was suspended in dry THF (59.60 mL). The resulted suspension was cooled down to −70° C. Then, a solution of n-BuLi (1.6 M in hexane, 14.91 mL) was added dropwise over 1 h at −70° C. via syringe pump. After addition completed, (3-bromopropoxy)(tert-butyl)dimethylsilane (8.21 mL, 35.77 mmol) was added over 1 h at −70° C. via syringe pump. The cooling bath was removed, and the mixture was allowed to warm up slowly to −10° C. (ice-bath with NaCl). The mixture was stirred at this temperature for 1 h. Then, the beige suspension became a clear brown solution. After 1 h, the solution was stirred one additional hour at ambient temperature before quenched with saturated NH4Cl solution. The mixture was extracted with CH₂Cl₂. The collected organic layers were washed with brine, dried over sodium sulfate, filtered, and concentrated under reduced pressure. The crude product was purified over silica (cyclohexane/EtOAc; 0-40%) to afford the product as white solid (1.91 g, 49%).

### (2R,3R,4R,5R)-2-((benzoyloxy) methyl)-5-(4-chloro-5-(3-hydroxypropyl)-7H-pyrrolo[2,3-d]pyrimidin-7-yl) tetrahydrofuran-3,4-diyl dibenzoate (417)

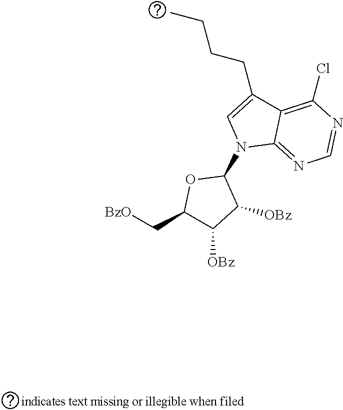

To a stirred suspension of compound 411 (1.90 g, 5.83 mmol) in MeCN (29.10 mL) was added BSA (1.58 mL, 6.41 mmol) at rt under nitrogen atmosphere. The suspension was stirred 10 min at ambient temperature during which time the reaction mixture became a clear solution. Then, 1-O-Acetyl-2,3,5-tri-O-benzoyl-B-D-ribofuranose (3.27 g, 6.41 mmol) was added followed by TMSOTf (1.17 mL, 6.41 mmol). The reaction mixture was heated to reflux (85° C.) and stirred 18 h before cooled down to rt. The mixture was diluted with EtOAc and the organic layer was washed with sat. bicarbonate solution and with brine. The organic layer was dried over sodium sulfate, filtered, and concentrated in vacuo. The obtained oil was purified by flash chromatography (cyclohexane/EtOAc; 0-50%) to afford a yellowish foam (1.13 g, 27%).

### (2R,3R,4R,5R)-2-((benzoyloxy) methyl)-5-(4-chloro-5-(3-(1,3-dioxoisoindolin-2-yl)propyl)-7H-pyrrolo[2,3-d]pyrimidin-7-yl) tetrahydrofuran-3,4-diyl dibenzoate (413)

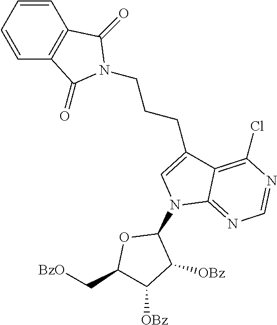

To a solution of 412 (1.10 g, 1.66 mmol) in dry THF (16.60 mL), phthatlimide (0.32 g, 2.16 mmol), PPh₃ (0.57 g, 2.16 mmol), and a solution of DEAD (40% in toluene, 0.94 g, 2.16 mmol) were added in this order. The solution was stirred magnetically at ambient temperature. The reaction mixture was diluted with CH₂Cl₂ and water. The organic layer was separated, and the aqueous phase was extracted with CH₂Cl₂. The collected organic layers were washed with brine, dried over sodium sulfate, and concentrated by rotatory evaporation. The obtained residue was purified over silica (cyclohexane/EtOAc; 0-50%) to afford the product as yellowish foam (0.82 g, 63%).

### (2R,35,4R,5R)-2-(hydroxymethyl)-5-(6,7,8,9-tetrahydro-2H-2,3,5,6-tetraazabenzo[cd]azulen-2-yl) tetrahydrofuran-3,4-diol (414)

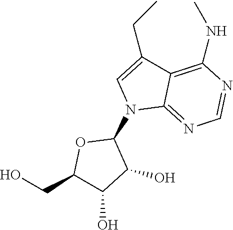 [SMILES: CCc1cn([C@@H]2O[C@H](CO)[C@@H](O)[C@H]2O)c2ncnc(NC)c12]

Compound 413 (0.61 g, 0.76 mmol) was suspended in absolute EtOH (38.10 mL). Then, ethylenediamine (0.25 mL, 1.53 mmol) was added and the mixture was heated to 50° C. The reaction mixture became after 20 min a clear solution and was stirred for 2 d at this temperature. Then, the reaction mixture was concentrated under reduced pressure. The obtained oil was redissolved in 1,4-dioxane (14.00 mL). Then, a solution of MeNH₂ (33 w % in EtOH, 14.00 mL) was added and the mixture was stirred at 60° C. for 2 d. After 2 d, the mixture was allowed to cool down to rt and concentrated under reduced pressure. The obtained crude product was purified over silica (CH₂Cl₂/MeOH; 0-20%) to give the product was white foam (0.06 g, 25%).

### 2-(((2R,35,4R,5R)-3,4-dihydroxy-5-(6,7,8,9-tetrahydro-2H-2,3,5,6-tetraazabenzo[cd]azulen-2-yl) tetrahydrofuran-2-yl)methyl) isoindoline-1,3-dione (415)

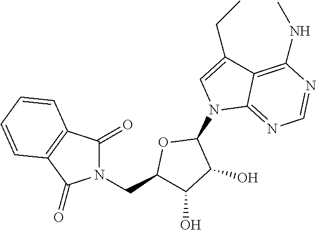 [SMILES: CCc1cn([C@@H]2O[C@H](CN3C(=O)c4ccccc4C3=O)[C@@H](O)[C@H]2O)c2ncnc(NC)c12]

To a solution of compound 414 (0.06 g, 0.18 mmol) in dry THF (3.60 mL) were added PPh₃ (0.19 g, 0.44 mmol), phthalimide (0.07 g, 0.44 mmol). The suspension was cooled down to −10° C. (ice-bath with NaCl). Then, DIAD (0.09 mL, 0.44 mmol) was added and stirred for 30 min at −10° C. Afterwards, the solution was allowed to warm up to rt. After 1 h, the solvents were removed in vacuo. The obtained residue was purififed over silica (CH₂Cl₂/MeOH; 0-10%) to afford the desired product as colorless foam (0.06 g, 82%).

### (2R,3S,4R,5R)-2-(aminomethyl)-5-(6,7,8,9-tetrahydro-2H-2,3,5,6-tetraazabenzo[cd]azulen-2-yl) tetrahydrofuran-3,4-diol (416)

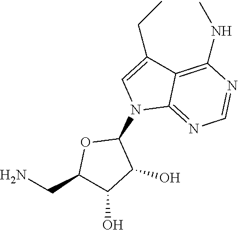 [SMILES: CCc1cn([C@@H]2O[C@H](CN)[C@@H](O)[C@H]2O)c2ncnc(NC)c12]

Hydrazine monohydrate (0.07 mL, 1.46 mmol) was added to a suspension of 415 (0.04 g, 0.09 mmol) in EtOH (2.30 mL) and stirred at 90° C. for 1 h. Note that the suspension became clear after approximately 5 min and before a white precipitate is formed after around 15 min. The reaction was cooled down in an ice-bath to rt. The precipitate was filtered off and the filter cake was washed with EtOH. The filtrate was concentrated under reduced pressure. The obtained residue was purified over amino coated silica (DCM/MeOH; 0-10%) to afford the desired product as colorless foam (0.02 g, 72%). ¹H NMR (400 MHZ, DMSO) δ8.01 (s, 1H, H2), 7.52 (t, J=3.7 Hz, 1H, —NH), 7.13 (s, 1H, H6), 6.02 (d, J=6.1 Hz, 1H, —OH), 5.23 (d, J=6.2 Hz, 1H, H1′), 5.08-5.01 (m, 1H, —OH), 4.34 (q, J=6.1 Hz, 1H, H2′), 4.06-4.01 (m, 1H, H3′), 3.77 (q, J=5.1 Hz, 1H, H4′), 3.41-3.28 (m, 2H, —CH₂CH₂CH₂NH-under water peak), 2.81 (t, J=5.6 Hz, 2H, —CH₂CH₂CH₂NH—), 2.74 (dd, J=20.4, 5.2 Hz, 1H, —C/2NH₂—), 2.53-2.46 (m, 2H, H5′ under DMSO peak), 1.93-1.86 (m, 2H, —CH₂CH₂CH₂NH—). HPLC: t_{R}=6.914 min (Method B). UV-purity at 210 nm=100.0%.

The synthesis of common intermediate A.

### Synthesis of ((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) methanol (Intermediate A)

### (1R,4S,5R,6S)-5,6-Dihydroxy-2-azabicyclo [2.2.1]heptan-3-one (1a)

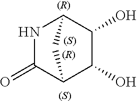

To a stirred solution of (1R,4S)-2-azabicyclo[2.2.1]hept-5-en-3-one (8.66 g, 79.4 mmol, 1.00 eq) and 4-methylmorpholine 4-oxide (10.23 g, 87.3 mmol, 1.1 eq) in 3-methylbutan-1-ol (40 mL) and H₂O (40 mL) was added OsO₄ (0.12 g, 0.48 mmol, 0.006 eq) in t-BuOH (2.0 mL) dropwise at room temperature and the reaction mixture was stirred for 2 h at 70° C. The resulting mixture was allowed to cool down to room temperature and was diluted with water (100 mL). The resulting mixture was extracted with AcOEt (3×100 mL). The combined organic layers were washed with brine and dried over anhydrous Na₂SO₄. After filtration, the filtrate was concentrated under reduced pressure. The crude residue was purified by silica gel column chromatography (90:10 DCM/MeOH) to afford (1R,4S,5R,6S)-5,6-dihydroxy-2-azabicyclo[2.2.1]heptan-3-one 1a (9.4 g, 83%) as a yellow solid. ¹H NMR (400 MHZ, Deuterium Oxide) 84.03 (d, J=5.9 Hz, 1H), 3.99 (d, J=5.9 Hz, 1H), 3.75 (p, J=1.6 Hz, 1H), 2.60 (d, J=1.9 Hz, 1H), 2.04 (d, J=1.7 Hz, 2H).

### Methyl(1S,2R,3S,4R)-4-amino-2,3-dihydroxycyclopentane-1-carboxylate hydrochloride (2a)

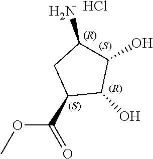

A solution of (1R,4S,5R,6S)-5,6-dihydroxy-2-azabicyclo[2.2.1]heptan-3-one 1a (9.4 g, 65.7 mmol, 1.0 eq) in HCl (g) in MeOH (100 mL) was stirred for 16 h at room temperature. The resulting mixture was concentrated under reduced pressure and the resulting residue was treated with AcOEt (50 mL). The precipitated solid was collected by filtration and washed with AcOEt (3×50 mL). The resulting solid was dried with an infared lamp. This resulted in methyl (1S,2R,3S,4R)-4-amino-2,3-dihydroxycyclopentane-1-carboxylate hydrochloride 2a (11.6 g, 83%) as a white solid. ¹H NMR (400 MHZ, Deuterium Oxide) 84.27 (t, J=5.3 Hz, 1H), 4.05 (ddd, J=7.3, 5.8, 1.7 Hz, 1H), 3.72 (s, 3H), 3.61-3.50 (m, 1H), 2.98 (td, J=9.0, 5.0 Hz, 1H), 2.50 (dt, J=13.8, 8.6 Hz, 1H), 1.83 (dt, J=13.7, 9.2 Hz, 1H).

### (1R,2S,3R,5R)-3-Amino-5-(hydroxymethyl) cyclopentane-1,2-diol (3a)

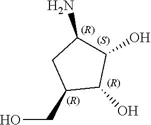

To a stirred solution of methyl(1S,2R,3S,4R)-4-amino-2,3-dihydroxycyclopentane-1-carboxylate hydrochloride 2 (9.0 g, 42.5 mmol, 1.0 eq) in THF (90 mL) was added lithium triethylborohydride (213 mL, 213 mmol, 5.0 eq) dropwise at 0° C. under N₂ atmosphere and the reaction mixture was stirred for 3 h at 0° C. The reaction was quenched by the addition of water (2 mL) at room temperature and HCl (4 M in MeOH) was added, dropwise, at room temperature. The precipitated solid was collected by filtration and washed with AcOEt (2×10 mL). The resulting solid was dried with an infrared lamp to afford (1R,2S,3R,5R)-3-amino-5-(hydroxymethyl) cyclopentane-1,2-diol hydrochloride 3a (8.50 g, crude) as a white solid.

### (1R,2S,3R,5R)-3-(4-Chloro-7H-pyrrolo[2,3-d]pyrimidin-7-yl)-5-(hydroxymethyl) cyclopentane-1,2-diol (4a)

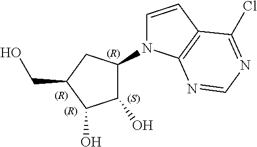

To a stirred solution of (1R,2S,3R,5R)-3-amino-5-(hydroxymethyl) cyclopentane-1,2-diol hydrochloride 3a (8.50 g, 46.3 mmol, 1.0 eq) and 2-(4,6-dichloropyrimidin-5-yl)acetaldehyde (9.73 g, 50.9 mmol, 1.1 eq) in EtOH (85 mL) were added TEA (14.1 g, 139 mmol, 3.0 eq) at room temperature under N₂ atmosphere. The resulting mixture was stirred for 3 h at 90° C. The mixture was allowed to cool down to room temperature. The resulting mixture was concentrated under reduced pressure to afford (1R,2S,3R,5R)-3-{4-chloropyrrolo[2,3-d]pyrimidin-7-yl}-5-(hydroxymethyl) cyclopentane-1,2-diol) as a brown oil 4a (10 g, crude). LCMS [conditions: XBridge C18, 50*3.0 mm, 3.5 μm, Mobile Phase A: Water/5 mM NH₄HCO₃, Mobile Phase B: Methanol, 10%-95% B-2 min (+), 1.50 mL/min, ES, m/z]: TR=1.05 min; [M+H]⁺: 284, 286

### ((3αR,4R,6R,6αS)-6-(4-Chloro-7H-pyrrolo[2,3-d]pyrimidin-7-yl)-2,2-dimethyltetrahydro-4H-cyclopenta[d][1,3]dioxol-4-yl) methanol (5a). (WO2017032840)

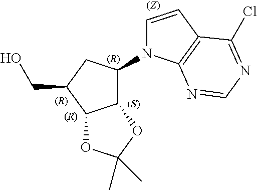

To a stirred solution of (1R,2S,3R,5R)-3-{4-chloropyrrolo[2,3-d]pyrimidin-7-yl}-5-(hydroxymethyl) cyclopentane-1,2-diol 4 (crude) and 2,2-dimethoxypropane (50 mL) in acetone (100 mL) were added TsOH (24 g) at room temperature. The reaction mixture was stirred for 2 h at room temperature, concentrated under reduced pressure and then suspended in water (200 mL). The resulting mixture was extracted with AcOEt (3×50 mL). The combined organic layers were washed with brine (50 mL), dried over anhydrous Na₂SO₄, filtered, and the filtrate was concentrated under reduced pressure. The crude residue was purified by silica gel column chromatography (1:1 Petroleum ether/AcOEt) to afford ((3αR,4R,6R,6αS)-6-(4-chloro-7H-pyrrolo[2,3-d]pyrimidin-7-yl)-2,2-dimethyltetrahydro-4H-cyclopenta[d][1,3]dioxol-4-yl) methanol 5a (5.3 g, 18% for 3 steps) as a yellow semi-solid. ¹H NMR (400 MHZ, DMSO-d₆) δ 8.65 (s, 1H), 7.96 (d, J=3.7 Hz, 1H), 6.72 (d, J=3.6 Hz, 1H), 5.10 (dt, J=12.8, 6.3 Hz, 1H), 4.92 (t, J=6.7 Hz, 1H), 4.56 (dd, J=7.3, 4.0 Hz, 1H), 3.53 (d, J=5.0 Hz, 2H), 2.26 (m, 2H), 2.18-2.04 (m, 1H), 1.49 (s, 3H), 1.23 (s, 3H). LCMS [conditions: Kinetex XB-C18, 50*3.0 mm, 2.6 μm, Mobile Phase A: Water/0.05% FA, Mobile Phase B: Acetonitrile/0.05% FA, 5%-100% B-2 min (+), 1.50 mL/min, ES, m/z]: TR=0.95 min; [M+H]⁺: 324, 326

### ((3αR,4R,6R,6αS)-6-(4-((4-Methoxybenzyl)(methyl) amino)-7H-pyrrolo[2,3-d]pyrimidin-7-yl)-2,2-dimethyltetrahydro-4H-cyclopenta[d][1,3]dioxol-4-yl) methanol (Intermediate A)

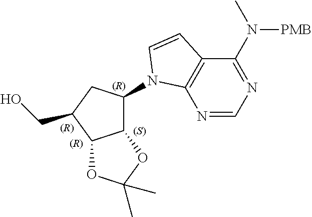

To a stirred solution of ((3αR,4R,6R,6αS)-6-(4-chloro-7H-pyrrolo[2,3-d]pyrimidin-7-yl)-2,2-dimethyltetrahydro-4H-cyclopenta[d][1,3]dioxol-4-yl) methanol 5a (1.50 g, 4.63 mmol, 1.0 eq) and [(4-methoxyphenyl)methyl](methyl) amine (1.05 g, 6.95 mmol, 1.5 eq) in EtOH (15 mL) was added TEA (0.94 g, 9.27 mmol, 2.0 eq) at room temperature and the reaction mixture was stirred for 16 h at 80° C. The resulting mixture was allowed to cool down to room temperature and was concentrated under reduced pressure. The crude residue was purified by silica gel column chromatography (AcOEt) to afford ((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) methanol Intermediate A (1.80 g, 89%) as an off-white solid. LCMS [conditions: Halo C18, 30*3.0 mm, 2.0 μm, Mobile Phase A: Water/0.05% TFA, Mobile Phase B: Acetonitrile, 5%-100% B-1.2 min (+), 1.50 L/min, ES, m/z]: TR=0.55 min; [M+H]⁺: 439. Chiral-HPLC [Column: YMC Cellulose-SB, 100*4.6 mm, 3 μm, Solvent B: Ethanol, Mobile Phase A: n-Hexane (0.1% DEA). Oven Temperature: 25° C., Total Flow: 3.00 mL/min]: TR=2.01 min; dr=>99:1.

### Synthesis of N-(((1R,2R,3S,4R)-2,3-dihydroxy-4-(4-(methylamino)-7H-pyrrolo[2,3-d]pyrimidin-7-yl) cyclopentyl)methyl)-N-methyl-3-(methylamino)propanamide (417)

### ((3αR,4R,6R,6αS)-6-(4-Chloro-7H-pyrrolo[2,3-d]pyrimidin-7-yl)-2,2-dimethyltetrahydro-4H-cyclopenta[d][1,3]dioxol-4-yl) methyl 4-methylbenzenesulfonate (ha)

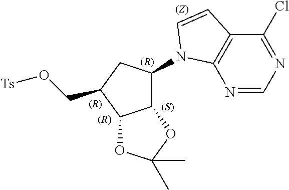

To a stirred solution of ((3αR,4R,6R,6αS)-6-(4-chloro-7H-pyrrolo[2,3-d]pyrimidin-7-yl)-2,2-dimethyltetrahydro-4H-cyclopenta[d][1,3]dioxol-4-yl) methanol 5a (200 mg, 0.62 mmol, 1.0 eq) and TsCl (141 mg, 0.74 mmol, 1.2 eq) in DCM (2.0 mL) was added TEA (125 mg, 1.24 mmol, 2.0 eq) and DMAP (15.1 mg, 0.12 mmol, 0.2 eq) and the reaction mixture was stirred for 16 h at room temperature. The crude reaction mixture was concentrated under reduced pressure and purified by silica gel column chromatography (3:1 Petroleum ether/AcOEt) to afford ((3αR,4R,6R,6αS)-6-(4-chloro-7H-pyrrolo[2,3-d]pyrimidin-7-yl)-2,2-dimethyltetrahydro-4H-cyclopenta[d][1,3]dioxol-4-yl)methyl-4-methylbenzenesulfonate 6a (200 mg, 68%) as a light yellow solid.

### 7-((3αS,4R,6R,6αR)-2,2-Dimethyl-6-((methylamino) methyl)tetrahydro-4H-cyclopenta[d][1,3]dioxol-4-yl)-N-methyl-7H-pyrrolo[2,3-d]pyrimidin-4-amine (7a)

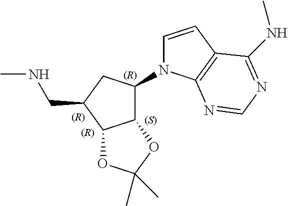

A mixture of ((3αR,4R,6R,6αS)-6-(4-chloro-7H-pyrrolo[2,3-d]pyrimidin-7-yl)-2,2-dimethyltetrahydro-4H-cyclopenta[d][1,3]dioxol-4-yl) methyl 4-methylbenzenesulfonate 6a (0.10 g, 0.21 mmol, 1.0 eq) and methylamine solution (30% in ethanol, 1.5 mL) was stirred overnight at room temperature. The resulting mixture was concentrated under reduced pressure and the crude residue was purified by reverse flash chromatography [column, C₁₈ silica gel; mobile phase: MeCN in water, 10% to 70% gradient in 13 min; detector, UV 254 nm] to afford 7-((3αS,4R,6R,6αR)-2,2-dimethyl-6-((methylamino) methyl)tetrahydro-4H-cyclopenta[d][1,3]dioxol-4-yl)-N-methyl-7H-pyrrolo[2,3-d]pyrimidin-4-amine 7a (25 mg, 36%) as a colorless oil. LCMS [conditions: XBridge C18, 50*3.0 mm, 3.5 μm, Mobile Phase A: Water/5 mM NH₄HCO₃, Mobile Phase B: Methanol, 10%-95% B-2 min (+), 1.50 mL/min, ES, m/z]: TR=1.08 min; [M+H]⁺: 332.

### tert-Butyl-(3-((((3αR,4R,6R,6αS)-2,2-dimethyl-6-(4-(methylamino)-7H-pyrrolo[2,3-d]pyrimidin-7-yl) tetrahydro-4H-cyclopenta[d][1,3]dioxol-4-yl)methyl)(methyl) amino)-3-oxopropyl)(methyl)carbamate (8a)

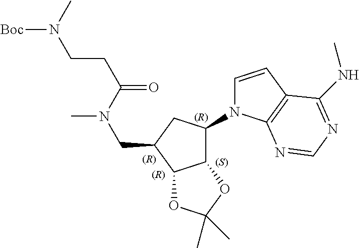

To a solution of 3-[(tert-butoxycarbonyl)(methyl) amino]propanoic acid (15 mg, 0.07 mmol, 1.0 eq) and triethylamine (11 mg, 0.11 mmol, 1.5 eq) in DCM (1.5 mL) was added HATU (34 mg, 0.09 mmol, 1.2 eq) and the mixture was stirred for 10 min at room temperature, under nitrogen. After this time, 7-((3αS,4R,6R,6αR)-2,2-dimethyl-6-((methylamino) methyl)tetrahydro-4H-cyclopenta[d][1,3]dioxol-4-yl)-N-methyl-7H-pyrrolo[2,3-d]pyrimidin-4-amine 7a (25 mg, 0.075 mmol, 1.0 eq) was added and the reaction mixture was stirred for 2 h at room temperature. The resulting crude mixture was evaporated and purified by silica gel column chromatography (9:1 DCM/MeOH) to afford tert-butyl-(3-((((3αR,4R,6R,6αS)-2,2-dimethyl-6-(4-(methylamino)-7H-pyrrolo[2,3-d]pyrimidin-7-yl) tetrahydro-4H-cyclopenta[d][1,3]dioxol-4-yl)methyl)(methyl) amino)-3-oxopropyl)(methyl)carbamate 8a (36 mg, 92%) as a colorless oil. LCMS [conditions: Xbridge Shield RP18, 50*3.0 mm, 3.5 μm, Mobile Phase A: Water/0.05% Ammonia, Mobile Phase B: Acetonitrile, 5%-100% B-2 min (+), 1.50 mL/min, ES, m/z]: TR=1.11 min; [M+H]⁺: 517.

### N-(((1R,2R,3S,4R)-2,3-Dihydroxy-4-(4-(methylamino)-7H-pyrrolo[2,3-d]pyrimidin-7-yl) cyclopentyl)methyl)-N-methyl-3-(methylamino) nronanamide (417)

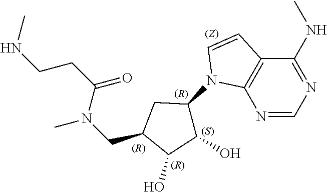

To a stirred solution of tert-butyl-(3-((((3αR,4R,6R,6αS)-2,2-dimethyl-6-(4-(methylamino)-7H-pyrrolo[2,3-d]pyrimidin-7-yl) tetrahydro-4H-cyclopenta[d][1,3]dioxol-4-yl)methyl)(methyl) amino)-3-oxopropyl)(methyl)carbamate 8a (36 mg, 0.07 mmol, 1.0 eq) in MeOH (1.0 mL) was added HCl (4M in MeOH, 1.0 mL) dropwise at 0° C. The reaction mixture was stirred overnight at room temperature. The resulting mixture was neutralized to pH=7-8 with ammonia water and was purified by Prep-HPLC [column, Xbridge Prep C18 OBD column, 5 μm, 19*150 mm; mobile phase, water (0.03% NH₄OH) and CH₃CN (20% CH₃CN up to 40% in 10 min); Detector, UV 220&254 nm] to afford N-{[(1R,2R,3S,4R)-2,3-dihydroxy-4-[4-(methylamino) pyrrolo[2,3-d]pyrimidin-7-yl]cyclopentyl]methyl}-N-methyl-3-(methylamino)propanamide 417 (11.3 mg, 42%) as a white solid. ¹H NMR (300 MHZ, DMSO-d₆) δ 8.12 (s, 1H), 7.38 (d, J=4.8 Hz, 1H), 7.25 (t, J=3.7 Hz, 1H), 6.54 (d, J=3.5 Hz, 1H), 4.91-4.74 (m, 1H), 4.30-4.18 (m, 1H), 3.82-3.69 (m, 1H), 3.65-3.51 (m, 1H), 3.35-3.19 (m, 1H), 2.99 (s, 2H), 2.96 (d, J=4.6 Hz, 3H), 2.83 (s, 1H), 2.79 (s, 1H), 2.66 (d, J=7.4 Hz, 1H), 2.51-2.34 (m, 1H), 2.33-2.01 (m, 4H), 1.79-1.22 (m, 1H). LCMS [conditions: ZORBAX SB-Aq, 50*4.6 mm, 1.8 μm, Mobile Phase A: Water/0.05% TFA, Mobile Phase B: Acetonitrile, 5%-30%-95% B-5 min, 1.50 mL/min, ES, m/z]: TR=1.36 min; [M+H]⁺: 377.

### Synthesis of N-(((1R,2R,3S,4R)-2,3-dihydroxy-4-(4-(methylamino)-7H-pyrrolo[2,3-d]pyrimidin-7-yl) cyclopentyl)methyl) piperidine-3-carboxamide (418)

### ((3αR,4R,6R,6αS)-2,2-Dimethyl-6-(4-(methylamino)-7H-pyrrolo[2,3-d]pyrimidin-7-yl) tetrahydro-4H-cyclopenta[d][1,3]dioxol-4-yl) methanol (9a)

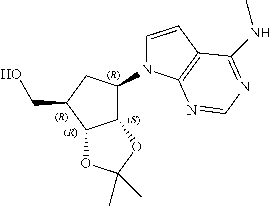

To ((3αR,4R,6R,6αS)-6-(4-chloro-7H-pyrrolo[2,3-d]pyrimidin-7-yl)-2,2-dimethyltetrahydro-4H-cyclopenta[d][1,3]dioxol-4-yl) methanol 5a (0.55 g, 1.70 mmol, 1.0 eq) was added methylamine (30% in ethanol, 4.0 mL) and the reaction mixture was stirred overnight at room temperature. The resulting mixture was diluted with AcOEt (10 mL) and the resulting solid was filtered and the filter cake was washed with AcOEt. The resulting filtrate was concentrated under reduced pressure to afford ((3αR,4R,6R,6αS)-2,2-dimethyl-6-(4-(methylamino)-7H-pyrrolo[2,3-d]pyrimidin-7-yl) tetrahydro-4H-cyclopenta[d][1,3]dioxol-4-yl) methanol 9a (0.50 g, 92%) as a light yellow solid. LCMS [conditions: Proshell HPH-C18, 50*3.0 mm, 2.7 μm, Mobile Phase A: Water/0.05% Ammonia, Mobile Phase B: Acetonitrile, 5%-100% B-2 min, 1.20 mL/min, ES, m/z]: TR=0.87 min; [M+H]⁺: 319.

### 2-(((3αR,4R,6R,6αS)-2,2-Dimethyl-6-(4-(methylamino)-7H-pyrrolo[2,3-d]pyrimidin-7-yl) tetrahydro-4H-cyclopenta[d][1,3]dioxol-4-yl)methyl) isoindoline-1,3-dione (10a)

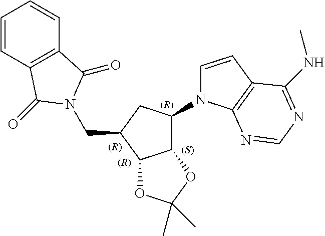

To a stirred mixture of ((3αR,4R,6R,6αS)-2,2-dimethyl-6-(4-(methylamino)-7H-pyrrolo[2,3-d]pyrimidin-7-yl) tetrahydro-4H-cyclopenta[d][1,3]dioxol-4-yl) methanol 9a (0.50 g, 1.57 mmol, 1.0 eq) and phthalimide (0.46 g, 3.14 mmol, 2.0 eq) and triphenylphosphine (0.82 g, 3.14 mmol, 2.0 eq) in THF was added DIAD (0.64 g, 3.14 mmol, 2.0 eq) dropwise, at 0° C., under nitrogen. The reaction mixture was stirred for 3 h at room temperature and the resulting mixture was concentrated under reduced pressure and was then purified by silica gel column chromatography (3:1 Petroleum ether/AcOEt) to afford 2-(((3αR,4R,6R,6αS)-2,2-dimethyl-6-(4-(methylamino)-7H-pyrrolo[2,3-d]pyrimidin-7-yl) tetrahydro-4H-cyclopenta[d][1,3]dioxol-4-yl)methyl) isoindoline-1,3-dione 10a (0.60 g, 85%) as a light yellow solid. LCMS [conditions: XBridge C18, 50*3.0 mm, 3.5 μm, Mobile Phase A: Water/5 mM NH₄HCO₃, Mobile Phase B: Methanol, 10%-95% B-2 min (+), 1.50 mL/min, ES, m/z]: TR=1.34 min; [M+H]⁺: 448.

### 7-((3αS,4R,6R,6αR)-6-(Aminomethyl)-2,2-dimethyltetrahydro-4H-cyclopenta[d][1,3]dioxol-4-yl)-N-methyl-7H-pyrrolo[2,3-d]pyrimidin-4-amine (11a)

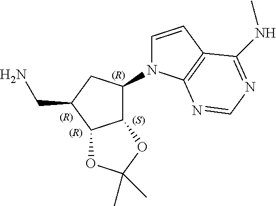

To a stirred solution of 2-(((3αR,4R,6R,6αS)-2,2-dimethyl-6-(4-(methylamino)-7H-pyrrolo[2,3-d]pyrimidin-7-yl) tetrahydro-4H-cyclopenta[d][1,3]dioxol-4-yl)methyl) isoindoline-1,3-dione 10a (0.60 g, 1.34 mmol, 1.0 eq) in ethanol (10 mL) was added hydrazine hydrate (80% aq., 0.13 g, 2.68 mmol, 2.0 eq) and the reaction mixture was stirred at 80° C. for 3 h. The resulting mixture was allowed to cool to room temperature, filtered and the filter cake was washed with ethanol. The filtrate was concentrated under reduced pressure to afford 7-((3αS,4R,6R,6αR)-6-(aminomethyl)-2,2-dimethyltetrahydro-4H-cyclopenta[d][1,3]dioxol-4-yl)-N-methyl-7H-pyrrolo[2,3-d]pyrimidin-4-amine 11a (0.40 g, 94%) as a colorless oil. LCMS [conditions: Xbridge Shield RP18, 50*3.0 mm, 3.5 μm, Mobile Phase A: Water/0.05% Ammonia, Mobile Phase B: Acetonitrile, 5%-100% B-2 min (+), 1.50, mL/min, ES, m/z]: TR=1.06 min; [M+H]⁺: 318.

### tert-Butyl 3-((((3αR,4R,6R,6αS)-2,2-dimethyl-6-(4-(methylamino)-7H-pyrrolo[2,3-d]pyrimidin-7-yl) tetrahydro-4H-cyclopenta[d][1,3]dioxol-4-yl) methyl)carbamoyl) piperidine-1-carboxylate (12a)

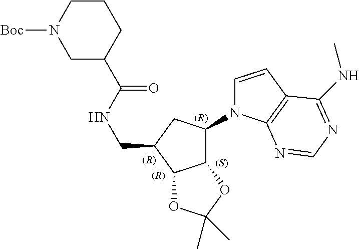

To a stirred solution of racemic 1-(tert-butoxycarbonyl) piperidine-3-carboxylic acid (36.1 mg, 0.16 mmol, 1.0 eq) in DCM (1.0 mL) were added TEA (31.9 mg, 0.32 mmol, 2.0 eq) and HATU (71.9 mg, 0.19 mmol, 1.2 eq) at room temperature. To the above mixture was then added 7-((3αS,4R,6R,6αR)-6-(aminomethyl)-2,2-dimethyltetrahydro-4H-cyclopenta[d][1,3]dioxol-4-yl)-N-methyl-7H-pyrrolo[2,3-d]pyrimidin-4-amine 11a (50 mg, 0.16 mmol, 1.00 eq) and the reaction mixture was stirred for 2 h at room temperature. The resulting mixture was purified by silica gel column chromatography (AcOEt) to afford tert-butyl 3-((((3αR,4R,6R,6αS)-2,2-dimethyl-6-(4-(methylamino)-7H-pyrrolo[2,3-d]pyrimidin-7-yl) tetrahydro-4H-cyclopenta[d][1,3]dioxol-4-yl)methyl)carbamoyl) piperidine-1-carboxylate 12a (50 mg, 60%) as an off-white solid. LCMS [conditions: Proshell HPH-C18, 50*3.0 mm, 2.7 μm, Mobile Phase A: Water/0.05% Ammonia, Mobile Phase B: Acetonitrile, 5%-100% B-2 min (+−), 1.20 mL/min, ES, m/z]: TR=1.13 min; [M+H]⁺: 529

### N-(((1R,2R,3S,4R)-2,3-dihydroxy-4-(4-(methylamino)-7H-pyrrolo[2,3-d]pyrimidin-7-yl) cyclopentyl)methyl) piperidine-3-carboxamide (418)

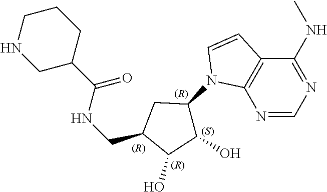

To tert-butyl-3-((((3αR,4R,6R,6αS)-2,2-dimethyl-6-(4-(methylamino)-7H-pyrrolo[2,3-d]pyrimidin-7-yl) tetrahydro-4H-cyclopenta[d][1,3]dioxol-4-yl)methyl)carbamoyl) piperidine-1-carboxylate 12a (50 mg, 0.08 mmol, 1.0 eq) was added TFA (1.0 mL) and the reaction mixture was stirred for 16 h at room temperature. The resulting mixture was concentrated under reduced pressure and the crude product was purified by Prep-HPLC [Xbridge Prep C18 OBD column, 5 μm, 19*150 mm; mobile phase, Water (0.03% NH₄OH) and CH₃CN (10% CH₃CN up to 35% in 8 min); Detector, UV 220&254 nm] to afford N-{[(1R,2R,3S,4R)-2,3-dihydroxy-4-[4-(methylamino) pyrrolo[2,3-d]pyrimidin-7-yl]cyclopentyl]methyl}piperidine-3-carboxamide 418 (25 mg, 83%) as a white solid. ¹H NMR (400 MHZ, DMSO-d₆) δ 8.12 (s, 1H), 8.01 (q, J=5.5 Hz, 1H), 7.38 (q, J=4.8 Hz, 1H), 7.22 (d, J=3.5 Hz, 1H), 6.54 (d, J=3.4 Hz, 1H), 6.12 (s, 1H), 4.81 (q, J=8.9 Hz, 1H), 4.19 (t, J=7.0 Hz, 1H), 4.09-3.61 (m, 3H), 3.27 (p,)=6.3 Hz, 1H), 3.12 (dq, J=17.3, 5.8 Hz, 1H), 2.96 (d, J=4.3 Hz, 3H), 2.83-2.56 (m, 2H), 2.32-1.97 (m, 3H), 1.79 (dd, J=13.0, 7.3 Hz, 1H), 1.66-1.42 (m, 3H), 1.32 (t, J=12.2 Hz, 1H). LCMS [conditions: L-column3 C18, 50*3.0 mm, 3 μm, Mobile Phase A: Water/0.05% Ammonia, Mobile Phase B: Acetonitrile, 5%-95% B-3 min (+), 1.20 mL/min, ES, m/z]: TR=0.85 min; [M+H]⁺: 389.

### Synthesis of 7-((3αS,4R,6R,6αR)-2,2-dimethyl-6-((2-(phenethylamino) ethoxy)methyl)tetrahydro-4H-cyclopenta[d][1,3]dioxol-4-yl)-N-(4-methoxybenzyl)-N-methyl-7H-pyrrolo[2,3-d]pyrimidin-4-amine (419)

### Methyl 2-(((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) methoxy)acetate (13a)

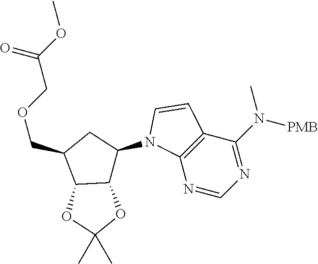

To a stirred solution of ((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) methanol (Intermediate A, 500 mg, 1.14 mmol, 1.0 eq) in THF (5.0 mL) was added NaH (41 mg, 1.71 mmol, 1.5 eq) in small portions at 0° C. To the reaction mixture was added methyl 2-bromoacetate (436 mg, 2.85 mmol, 2.5 eq) dropwise over 5 min at 0° C. and the resulting mixture was stirred for additional 12 h at room temperature and then quenched with water at 0° C. The resulting mixture was concentrated under reduced pressure and the crude residue was purified by silica gel column chromatography (3:1 Petroleum ether/AcOEt) to afford methyl 2-(((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) methoxy)acetate 13a (200 mg, 27%) as a white solid. ¹H NMR (400 MHz, DMSO-d₆) δ 8.18 (s, 1H), 7.37 (d, J=3.6 Hz, 1H), 7.19 (d, J=8.3 Hz, 2H), 6.92-6.84 (m, 2H), 6.58 (t, J=4.0 Hz, 1H), 5.04 (dt, J=12.3, 6.6 Hz, 1H), 4.94 (s, 2H), 4.90 (t, J=6.7 Hz, 1H), 4.56 (dd, J=7.2, 4.7 Hz, 1H), 4.17 (s, 2H), 3.72 (s, 3H), 3.67 (s, 3H), 3.65-3.53 (m, 2H), 3.28 (s, 3H), 2.35 (p, J=5.9 Hz, 1H), 2.24 (dt, J=13.3, 6.7 Hz, 1H), 2.11-1.97 (m, 1H), 1.48 (s, 3H), 1.23 (s, 3H). LCMS [column: Xbridge C18,50*3.0 mm, 3.5 μm, mobile phase A: Water/5 mM NH₄HCO₃, Mobile Phase B: Methanol, 10%-95% B-3 min (+), 1.50 mL/min]: TR=2.14 min; [M+H]⁺: 511.2.

### 2-(((3αR,4R,6R,6αS)-6-(4-((4-Methoxybenzyl)(methyl) amino)-7H-pyrrolo[2,3-d]pyrimidin-7-yl)-2,2-dimethyltetrahydro-4H-cyclopenta[d][1,3]dioxol-4-yl) methoxy)ethan-1-ol (14a)

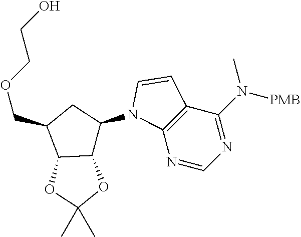

To a stirred solution of methyl 2-(((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) methoxy)acetate 13a (200 mg, 0.39 mmol, 1.0 eq) in THF (5 mL) was added LiAlH₄ (29.7 mg, 0.78 mmol, 2.0 eq) in several portions at 0° C. and the reaction mixture was stirred at 0° C. for. The reaction was quenched by the addition of crystalline sodium sulfate (1.0 g) at 0° C., the mixture was filtered and the filtrate was concentrated under reduced pressure. The crude residue was purified by silica gel column chromatography (AcOEt) to afford 2-(((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) methoxy)ethan-1-ol 14a (150 mg, 79%) as a white solid. LCMS [column: Xbridge Shield RP18, 50*3.0 mm, 3.5 μm, Mobile Phase A: Water/0.05% Ammonia, Mobile Phase B: Acetonitrile, 10%-95% B-3 min (+), 1.50 mL/min, ES, m/z]: TR=1.59 min; [M+H]⁺: 483.3.

### 2-(((3αR,4R,6R,6αS)-6-(4-((4-Methoxybenzyl)(methyl) amino)-7H-pyrrolo[2,3-d]pyrimidin-7-yl)-2,2-dimethyltetrahydro-4H-cyclopenta[d][1,3]dioxol-4-yl) methoxy) ethyl methanesulfonate (15a)

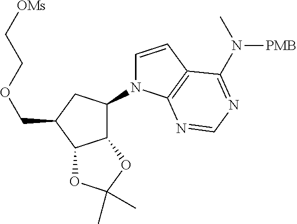

To a stirred solution of 2-(((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) methoxy)ethan-1-ol 14a (150 mg, 0.31 mmol, 1.0 eq) and TEA (62.9 mg, 0.62 mmol, 2.0 eq) in DCM (5 mL) was added MsCl (39.2 mg, 0.34 mmol, 1.1 eq) dropwise at 0° C. and the reaction mixture was stirred at 0° C. for 2 h. The resulting mixture was concentrated under reduced pressure to afford 2-(((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) methoxy) ethyl methanesulfonate 15a (300 mg, crude), which was used in the next step directly without further purification. LCMS [column: XBridge C18, 50*3.0 mm, 3.5 μm, Mobile Phase A: Water/5 mM NH₄HCO₃, Mobile Phase B: Methanol, 10%-95% B-3 min (+), 1.50 mL/min]: TR=2.09 min; [M+H]⁺: 561.2.

### 7-((3αS,4R,6R,6αR)-2,2-Dimethyl-6-((2-(phenethylamino) ethoxy)methyl)tetrahydro-4H-cyclopenta[d][1,3]dioxol-4-yl)-N-(4-methoxybenzyl)-N-methyl-7H-pyrrolo[2,3-d]pyrimidin-4-amine (16a)

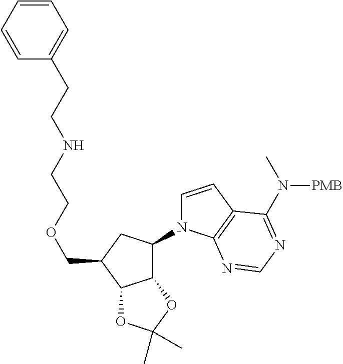

To a stirred solution of 2-(((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) methoxy) ethyl methanesulfonate 15a (70 mg, 0.13 mmol, 1.0 eq) and 2-phenylethylamine (22.7 mg, 0.19 mmol, 1.50 eq) in dioxane (3 mL) was added NaH (3.6 mg, 0.15 mmol, 1.1 eq) in several portions at 0° C. and the reaction mixture was stirred at 100° C. for 16 h. The resulting mixture was allowed to cool to room temperature and was quenched with water (0.5 mL). The crude residue was purified by reverse flash chromatography [C₁₈ silica gel; mobile phase, ACN in water, 10% to 80% gradient in 15 min; detector, UV 220 nm]: to afford 7-((3αS,4R,6R,6αR)-2,2-dimethyl-6-((2-(phenethylamino) ethoxy)methyl)tetrahydro-4H-cyclopenta[d][1,3]dioxol-4-yl)-N-(4-methoxybenzyl)-N-methyl-7H-pyrrolo[2,3-d]pyrimidin-4-amine 16a (65 mg, 89%) as a yellow solid. LCMS [column: Xbridge Shield RP18, 50*3.0 mm, 3.5 μm, Mobile Phase A: Water/0.05% Ammonia, Mobile Phase B: Acetonitrile, 10%-95% B-3 min (+), 1.50 mL/min, ES, m/z]: TR=2.01 min; [M+H]⁺: 586.3.

### (1R,2S,3R,5R)-3-[4-(Methylamino) pyrrolo[2,3-d]pyrimidin-7-yl]-5-({2-[(2-phenylethyl)amino]ethoxy}methyl) cyclopentane-1,2-diol (419)

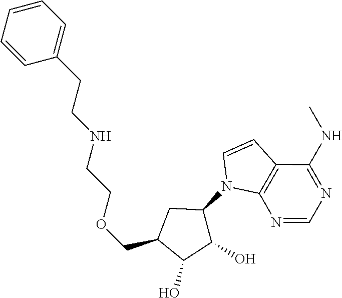 [SMILES: CNc1ncnc2c1ccn2[C@@H]1C[C@H](COCCNCCc2ccccc2)[C@@H](O)[C@H]1O]

To 7-((3αS,4R,6R,6αR)-2,2-dimethyl-6-((2-(phenethylamino) ethoxy)methyl)tetrahydro-4H-cyclopenta[d][1,3]dioxol-4-yl)-N-(4-methoxybenzyl)-N-methyl-7H-pyrrolo[2,3-d]pyrimidin-4-amine 16a (65 mg, 0.11 mmol) was added TFA (2.0 mL) and the reaction mixture was stirred for 2 h at room temperature. The resulting mixture was concentrated under reduced pressure and was purified by reverse flash chromatography [C₁₈ silica gel; mobile phase, ACN in water, 30% to 50% gradient in 10 min; detector, UV 254 nm, 220 nm]: to afford (1R,2S,3R,5R)-3-[4-(methylamino) pyrrolo[2,3-d]pyrimidin-7-yl]-5-({2-[(2-phenylethyl)amino]ethoxy}methyl) cyclopentane-1,2-diol (419, 10 mg, 21%) as a white solid. 1H NMR (400 MHZ, DMSO-d₆) δ 8.12 (s, 1H), 7.37 (q, J=4.8 Hz, 1H), 7.30-7.07 (m, 6H), 6.54 (d, J=3.4 Hz, 1H), 4.87 (q, J=8.9 Hz, 1H), 4.19 (dd, J=8.4, 5.5 Hz, 1H), 3.80 (s, 1H), 3.49-3.29 (m, 5H), 3.21-3.10 (m, 1H), 2.96 (d, J=4.5 Hz, 3H), 2.84-2.65 (m, 3H), 2.60 (t, J=7.0 Hz, 1H), 2.25-2.06 (m, 2H), 1.78-1.60 (m, 2H), 1.59-1.46 (m, 1H). LCMS (column HPH-C18,50*3.0 mm2.7 μm. mobile phase A: water/0.05% ammonia water, mobile phase B: Acetonitrile, 5%-95% B-3.0 min (+).Icm, 1.50 mL/min, ES, m/z): TR=1.36 min; [M+H]⁺: 426.2. Chiral-SFC [Column Name: SC 100×4.6 mm 3.0 μm Co Solvent: B: MeOH (20 mM NH₃), Start Conc. of Pump B: 30.0%; Oven Temperature: 35° C.; Total Flow: 3.00 mL/min; BPR Pressure: 15.00 MPa; BPR Temperature: 50° C.]: TR=1.98 min (4 min run), single peak.

### Synthesis of (1R,2S,3R,5R)-3-(4-(methylamino)-7H-pyrrolo[2,3-d]pyrimidin-7-yl)-5-((3-(phenethylamino)propoxy) methyl) cyclopentane-1,2-diol (420)

### 3-(Phenethylamino)propan-1-ol (56a)

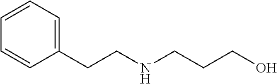 [SMILES: OCCCNCCc1ccccc1]

A mixture of 2-bromoethyl benzene (5.00 g, 27.0 mmol, 1.0 eq) and propanolamine (12.2 g, 162 mmol, 6.0 eq) in ethanol (30 mL) was heated at 80° C. for 3 h. The reaction mixture was then concentrated to remove ethanol and the resulting residue was diluted with DCM (100 mL). The organic layer was partitioned with water (100 mL) and the aqueous layer was extracted with DCM (120 mL). The combined organic layers were washed with brine (10 mL), dried over sodium sulfate, filtered, and concentrated to afford 3-(phenethylamino)propan-1-ol 56 (4.50 g, 85%) as a light yellow oil. ¹H NMR (300 MHZ, DMSO-d₆) δ 7.35-7.24 (m, 2H), 7.23-7.12 (m, 3H), 3.46 (t, J=6.3 Hz, 2H), 2.70 (q, J=5.0, Hz, 4H), 2.60 (t, J=6.8 Hz, 2H), 1.55 (p, J=6.6 Hz, 2H).

### tert-Butyl-(3-hydroxypropyl)(phenethyl)carbamate (57a)

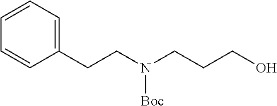 [SMILES: CC(C)(C)OC(=O)N(CCCO)CCc1ccccc1]

To a stirred mixture of 3-(phenethylamino)propan-1-ol 56 (4.50 g, 25.1 mmol, 1.0 eq) and Et₃N (5.10 g, 50.3 mmol, 2.0 eq) in DCM (80 mL) was added di-tert-butyl dicarbonate (6.60 g, 30.2 mmol, 1.2 eq) in several portions, at 0° C. and the reaction mixture was stirred for 3 h at room temperature. The reaction was quenched with water (100 mL) at room temperature and the resulting mixture was extracted with CH₂Cl₂ (60 mL). The organic layer were dried over anhydrous Na₂SO₄, filtered and the filtrate was concentrated under reduced pressure. The crude residue was purified by silica gel column chromatography (6:1 Petroleum ether/AcOEt) to afford tert-butyl-(3-hydroxypropyl)(phenethyl)carbamate 57a (5.00 g, 71%) as a colorless oil. 1H NMR (300 MHZ, DMSO-d₆) δ 7.35-7.25 (m, 2H), 7.24-7.15 (m, 3H), 4.41 (t, J=5.1 Hz, 1H), 3.44-3.28 (m, 4H), 3.16 (t, J=7.2 Hz, 2H), 2.76 (dd, J=8.6, 6.4 Hz, 2H), 1.68-1.53 (m, 2H), 1.36 (s, 9H).

### tert-Butyl-(3-bromopropyl)(phenethyl)carbamate (58a)

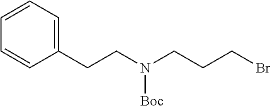 [SMILES: CC(C)(C)OC(=O)N(CCCBr)CCc1ccccc1]

To a stirred solution of tert-butyl-(3-hydroxypropyl)(phenethyl)carbamate 57 (1.00 g, 3.58 mmol, 1.0 eq) and triphenylphosphine (1.13 g, 4.30 mmol, 1.2 eq) in DCM was added NBS (0.76 g, 4.30 mmol, 1.2 eq) in several portions, at 0° C., under nitrogen. The resulting mixture was stirred for 1.5 h at 0° C. before the reaction was quenched by the addition of water (20 mL) at room temperature. The resulting mixture was extracted with DCM (30 mL) and the organic layer was washed with brine (15 mL), dried over anhydrous Na₂SO₄, filtered and the filtrate was concentrated under reduced pressure. The crude residue was purified by silica gel column chromatography (10:1 Petroleum ether/AcOEt) to afford tert-butyl (3-bromopropyl)(phenethyl)carbamate 58a (0.4 g, 33%) as a colorless oil.

### tert-Butyl-(3-(((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) methoxy)propyl)(phenethyl)carbamate (59a)

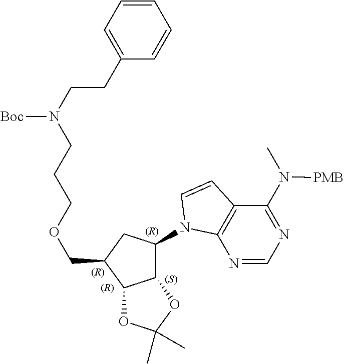

To a stirred solution of ((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) methanol (Intermediate A, 50 mg, 0.11 mmol, 1.0 eq) in THF (1 mL) was added NaH (6.8 mg, 0.17 mmol, 1.5 eq) in several portions at 0° C. and the reaction mixture was stirred at 0° C. for 10 min. After this time tert-butyl-(3-bromopropyl)(phenethyl)carbamate 58a (97.5 mg, 0.29 mmol, 2.5 eq) was added and the resulting mixture was stirred for 16 h at room temperature. The reaction mixture was quenched with water (0.2 mL) and purified by silica gel column chromatography (1:1 Petroleum ether/AcOEt) to afford tert-butyl-(3-(((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) methoxy)propyl)(phenethyl)carbamate 59 (45 mg, 56%) as a light yellow solid. LCMS [conditions: Kinetex XB-C18, 50*3.0 mm, 2.6 μm, Mobile Phase A: Water/0.05% FA, Mobile Phase B: Acetonitrile/0.05% FA, 5%-100% B-2.0 min (+−), 1.50 mL/min, ES, m/z]: TR=1.44 min; [M+H]⁺: 600.

### (1R,2S,3R,5R)-3-(4-(Methylamino)-7H-pyrrolo[2,3-d]pyrimidin-7-yl)-5-((3-(phenethylamino)propoxy) methyl) cyclopentane-1.2-diol (420)

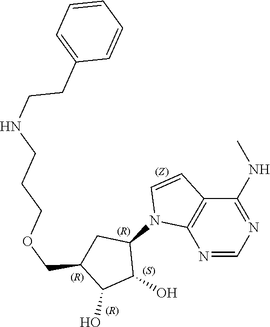

To tert-butyl-(3-(((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) methoxy)propyl)(phenethyl)carbamate 59a (45 mg, 0.064 mmol, 1.0 eq) was added trifluoroacetaldehyde (1.0 mL) and the reaction mixture was stirred for 16 h at room temperature. The resulting mixture was concentrated under reduced pressure and the crude product was purified by Prep-HPLC [Column, XBridge Shield RP18 OBD Column, 19*150 mm, 5 μm; mobile phase, Water (10 mmol/L NH₄HCO₃+0.1% NH₃.H₂O) and ACN (20% ACN up to 74% in 7 min); Detector, UV 220 nm]: to afford (1R,2S,3R,5R)-3-(4-(methylamino)-7H-pyrrolo[2,3-d]pyrimidin-7-yl)-5-((3-(phenethylamino)propoxy) methyl) cyclopentane-1,2-diol 420 (20 mg, 70%) as an off-white solid. ¹H NMR (400 MHZ, DMSO-d₆) δ 8.11 (s, 1H), 7.49-7.06 (m, 6H), 6.53 (d, J=3.5 Hz, 1H), 4.86 (q, J=8.9 Hz, 1H), 4.16 (dd, J=8.6, 5.2 Hz, 1H), 3.92-3.72 (m, 1H), 3.70-3.33 (m, 8H), 3.14 (s, 1H), 2.68 (s, 3H), 2.58 (t, J=6.9 Hz, 1H), 2.29-2.03 (m, 2H), 1.66 (q, J=6.8 Hz, 2H), 1.52 (q, J=10.3 Hz, 1H). LCMS [conditions: Halo C18, 50*3.0 mm, 2.7 μm, Mobile Phase A: Water/0.05% FA, Mobile Phase B: Acetonitrile, 5%-30%-95% B-3 min, 1.50 mL/min, ES, m/z]: TR=1.14 min; [M+H]⁺: 440.

### Synthesis of (1R,2S,3R,5R)-3-(4-(methylamino)-7H-pyrrolo[2,3-d]pyrimidin-7-yl)-5-((3-((3-phenoxyphenethyl) a mino)propoxy) methyl) cyclopentane-1,2-diol (421)

### 1-(2-Methoxyvinyl)-3-phenoxybenzene (44a)

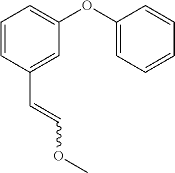 [SMILES: COC=Cc1cccc(Oc2ccccc2)c1]

To (methoxymethyl) triphenylphosphanium (93.0 g, 302 mmol, 4.0 eq) in dry THF (300 mL), at 0° C., under nitrogen, was added t-BuOK (34 g, 302 mmol, 4.0 equiv) and the reaction mixture was stirred for 45 minutes at 0° C. After this time, 3-phenoxybenzaldehyde (15 g, 75.7 mmol, 1.0 eq) was added dropwise, at 0° C., and the resulting mixture was stirred at room temperature for 3 h. The reaction was quenched with saturated NH₄Cl and extracted with EtOAc (300 mL). The organic phase was dried with Na₂SO₄, filtered, and concentrated. The crude residue was purified by silica gel column chromatography (Petroleum ether/AcOEt=30/1) to give 1-(2-methoxyvinyl)-3-phenoxybenzene 44a (12 g, 70%) as a colorless oil. LCMS [column: Kinetex XB-C18, 50*3.0 mm, 2.6 μm, Mobile Phase A: Water/0.05% TFA, Mobile Phase B: Acetonitrile, 5%-100% B-2 min (+), 1.50 mL/min, ES, m/z]: TR=1.29 min; [M+H]⁺: 227.

### 2-(3-Phenoxyphenyl)acetaldehyde (45a)

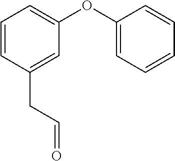 [SMILES: O=CCc1cccc(Oc2ccccc2)c1]

To a solution of 1-(2-methoxyvinyl)-3-phenoxybenzene 44a (8.0 g, 35.4 mmol, 1.0 eq) in THF (270 mL) was added HCl (5 M, 110 mL), and the reaction mixture was stirred at 70° C. for 30 min. The resulting mixture was diluted with EtOAc (300 mL) and washed with H₂O (100 ml×2). The organic phase was dried over Na₂SO₄ and concentrated to afford 2-(3-phenoxyphenyl)acetaldehyde 45a (6.10 g, 81%) as a light yellow oil. ¹H NMR (400 MHZ, Chloroform-d) 8 9.76 (t, J=2.3 Hz, 1H), 7.38-7.34 (m, 3H), 7.15 (t, J=7.4 Hz, 1H), 7.08-7.03 (m, 2H), 6.99-6.95 (m, 2H), 6.90 (d, J=2.1 Hz, 1H), 3.68 (d, J=2.3 Hz, 2H).

### 3-((3-Phenoxyphenethyl) amino)propan-1-ol (46a)

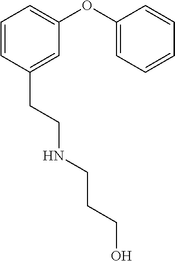 [SMILES: OCCCNCCc1cccc(Oc2ccccc2)c1]

To a solution 2-(3-phenoxyphenyl)acetaldehyde 45a (4.00 g, 18.8 mmol, 1.0 eq) in DCM (40 mL) was added propanolamine (1.70 g, 22.6 mmol, 1.2 eq), at room temperature, and the reaction mixture was stirred at room temperature, under nitrogen, overnight. The resulting mixture was concentrated under reduced pressure, the crude residue was dissolved in MeOH (100 mL) and then Pd/C(0.20 g, 10%) was added, under nitrogen. The resulting mixture was hydrogenated at room temperature for 3.0 h under a hydrogen balloon, filtered through a Celite pad and concentrated under reduced pressure to afford 3-((3-phenoxyphenethyl) amino)propan-1-ol 46a (4.60 g, 90%) as a light yellow oil, which was used in the next step without further purification. LCMS [Kinetex XB-C18, 3.0*30 mm, 1.7 μm, Mobile Phase A: Water/0.1% FA, Mobile Phase B: Acetonitrile/0.05% FA, 5%-100% B-1.2 min (+), 1.20 mL/min, ES, m/z]: TR=0.53 min; [M+H]⁺: 271.

### tert-Butyl-(3-hydroxypropyl)(3-phenoxyphenethyl) carhamate (47a)

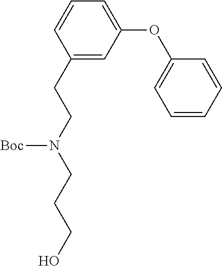 [SMILES: CC(C)(C)OC(=O)N(CCCO)CCc1cccc(Oc2ccccc2)c1]

To a stirred solution of 3-((3-phenoxyphenethyl) amino)propan-1-ol 46a (4.60 g, 17.0 mmol, 1.0 eq) and TEA (5.15 g, 50.9 mmol, 3.0 eq) in DCM (100 mL) was added Boc₂O (4.44 g, 20.3 mmol, 1.2 eq) in several portions, at room temperature, under nitrogen and the reaction mixture was stirred overnight. The resulting mixture was diluted with water (50 mL) and extracted with DCM (200 mL). The organic phase was washed with brine (50 mL), dried over anhydrous Na₂SO₄. After filtration, the filtrate was concentrated under reduced pressure and the crude residue was purified by silica gel column chromatography (Petroleum ether: AcOEt, 3:1) to afford tert-butyl-(3-hydroxypropyl)(3-phenoxyphenethyl)carbamate 47a (3.20 g, 51%) as a colorless oil. ¹H NMR (400 MHz, Chloroform-d) δ 7.36 (t, J=7.8 Hz, 2H), 7.26 (d, J=7.8 Hz, 1H), 7.13 (t, J=7.4 Hz, 1H), 7.03 (d, J=8.0 Hz, 2H), 6.95-6.82 (m, 3H), 3.55 (s, 2H), 3.37 (d, J=7.2 Hz, 4H), 2.82 (t, J=7.5 Hz, 2H), 1.65 (s, 2H), 1.46 (s, 9H). LCMS [Column: Proshell HPH-C18, 50*3.0 mm, 2.7 μm, Mobile Phase A: Water/0.05% Ammonia, Mobile Phase B: Acetonitrile, 5%-100% B-2.0 min (+), 1.20 mL/min, ES, m/z]: TR=1.39 min; [M+H]⁺: 371.

### tert-Butyl-(3-bromopropyl)(3-phenoxyphenethyl)carbamate (48a)

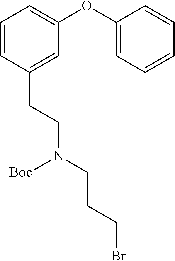 [SMILES: CC(C)(C)OC(=O)N(CCCBr)CCc1cccc(Oc2ccccc2)c1]

To a stirred mixture of tert-butyl-(3-hydroxypropyl)(3-phenoxyphenethyl)carbamate 47a (250 mg, 0.67 mmol, 1.0 eq) and CBr4 (267.8 mg, 0.81 mmol, 1.2 eq) in DCM (5.0 mL) was added PPh₃ (211 mg, 0.81 mmol, 1.2 eq) at room temperature under nitrogen and the reaction mixture was stirred for 3 h at room temperature. The resulting mixture was concentrated under reduced pressure and the crude residue was purified by silica gel column chromatography (5:1 Petroleum ether/AcOEt) to afford tert-butyl-(3-bromopropyl)(3-phenoxyphenethyl)carbamate 48a (250 mg, 86%) as a light yellow oil. LCMS [Kinetex XB-C18, 3.0*30 mm, 1.7 μm, Mobile Phase A: Water/0.1% FA, Mobile Phase B: Acetonitrile/0.05% FA, 5%-100% B-1.2 min (+), 1.20 mL/min, ES, m/z]: TR=0.90 min; [M+H]⁺: 434.

### tert-Butyl-(3-(((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4yl) methoxy)propyl)(3phenoxyphenethyl)carbamate (49a)

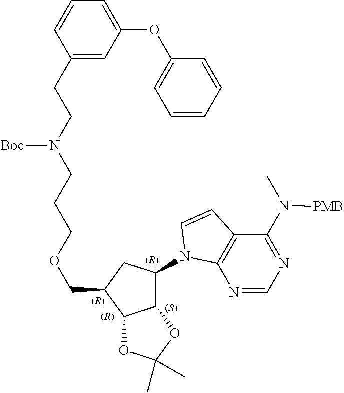

To a solution of ((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) methanol (Intermediate A, 151 mg, 0.35 mmol, 1.0 eq) in DMF (2.0 mL) was added NaH (60% in minerol oil, 16.6 mg, 0.41 mmol, 1.2 eq) at 0° C. The resulting mixture was stirred for 30 min at 0° C., before adding tert-butyl (3-bromopropyl)(3-phenoxyphenethyl)carbamate 48a (150 mg, 0.35 mmol, 1.0 eq) in DMF (1.0 mL) and the reaction mixture was stirred for 16 h at room temperature.

The reaction was quenched with water (10 mL) at 0° C. and extracted with DCM (75 mL). The organic layer was washed with brine (20 mL) and dried over anhydrous Na₂SO₄, filtered and concentrated under reduced pressure. The crude residue was purified by silica gel column chromatography (5:1 Petroleum ether/AcOEt) to afford tert-butyl (3-(((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) methoxy)propyl)(3-phenoxyphenethyl)carbamate 49a (60 mg, 23%) as a light yellow solid. ¹H NMR (400 MHZ, Chloroform-d) 8 8.43 (s, 1H), 7.54 (s, 1H), 7.34 (t, J=7.8 Hz, 2H), 7.20 (d, J=8.1 Hz, 3H), 7.11 (s, 1H), 7.00 (d, J=7.7 Hz, 3H), 6.88-6.83 (m, 4H), 6.49 (d, J=3.2 Hz, 1H), 5.15-5.09 (m, 1H), 5.03-4.98 (m, 2H), 4.92-4.87 (m, 1H), 4.64-4.59 (m, 1H), 3.81 (s, 3H), 3.57-3.53 (m, 2H), 3.49-3.46 (m, 2H), 3.40-3.34 (m, 4H), 2.85-2.76 (m, 2H), 2.50-2.41 (m, 2H), 2.25-2.19 (d, J=12.1 Hz, 1H), 1.85-1.74 (m, 2H), 1.59 (s, 3H), 1.56 (s, 3H), 1.44 (s, 9H), 1.28 (s, 3H), 1.02-0.86 (m, 3H). LCMS [Halo C18, 30*3.0 mm, 2.0 μm, Mobile Phase A: Water/0.05% TFA, Mobile Phase B: Acetonitrile, 5%-100% B-1.2 min (+), 1.20 mL/min, ES, m/z]: TR=0.82 min; [M+H]⁺: 792.

### (1R,2S,3R,5R)-3-(4-(Methylamino)-7H-pyrrolo[2,3-d]pyrimidin-7-yl)-5-((3-((3-phenoxyphenethyl) amino)propoxy) methyl) cyclopentane-1,2-diol (421)

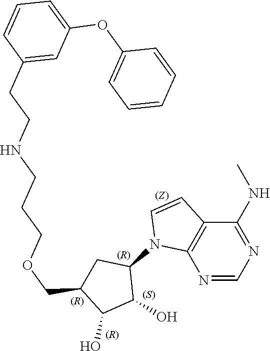

To tert-butyl-(3-(((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) methoxy)propyl)(3-phenoxyphenethyl)carbamate 49a (60 mg, 0.076 mmol, 1.0 eq) was added TFA (6.0 mL) and the reactin mixture was stirred at room temperature, under nitrogen, overnight. The resulting mixture was concentrated under reduced pressure and the crude residue was dissolved in MeOH (5 mL) and purified by prep-HPLC [Column: Sunfire C18 OBD; Mobile phase: A: water (0.01% NH₃.H₂O); B: ACN; Gradient: 32-53% B in 5 min; detector: 220 nm; flow rate: 20 ml/min]: to afford (1R,2S,3R,5R)-3-(4-(methylamino)-7H-pyrrolo[2,3-d]pyrimidin-7-yl)-5-((3-((3-phenoxyphenethyl) amino)propoxy) methyl) cyclopentane-1,2-diol 421 (16.2 mg, 38%) as a pale yellow solid. ¹H NMR (400 MHZ, DMSO-d₆) δ 8.11 (s, 1H), 7.38 (t, J=7.9 Hz, 3H), 7.27 (t, J=7.9 Hz, 1H), 7.20 (d, J=3.4 Hz, 1H), 7.12 (t, J=7.4 Hz, 1H), 6.98 (t, J=8.4 Hz, 3H), 6.86 (s, 1H), 6.80 (d, J=8.2 Hz, 1H), 6.53 (d, J=3.2 Hz, 1H), 5.33 (t, J=4.9 Hz, 1H), 4.86 (q, J=8.9 Hz, 1H), 4.78 (d, J=6.5 Hz, 1H), 4.64 (s, 1H), 4.23-4.13 (m, 1H), 3.81-3.77 (m, 1H), 3.48-3.35 (m, 4H), 2.98-2.93 (m, 2H), 2.73-2.65 (m, 2H), 2.60-2.56 (m, 1H), 2.24-2.08 (m, 1H), 2.04-1.98 (m, 1H), 1.67-1.60 (m, 2H), 1.58-1.42 (m, 2H), 1.34-1.27 (m, 2H), 0.90-0.83 (m, 1H). LCMS [Halo C18, 50*3.0 mm, 2.7 μm, Mobile Phase A: Water/0.05% TFA, Mobile Phase B: Acetonitrile, 5%-50%-95% B-3 min, 1.50 mL/min, ES, m/z]: TR=1.19 min; [M+H]⁺: 532.

### Synthesis of (1R,2S,3R,5R)-3-(4-(methylamino)-7H-pyrrolo[2,3-d]pyrimidin-7-yl)-5-((E)-4-(phenethylamino) but-1-en-1-yl) cyclopentane-1,2-diol (422)

### (3-((tert-Butoxycarbonyl)(phenethyl) amino) pronyl) trinhenylphosphonium bromide (60a)

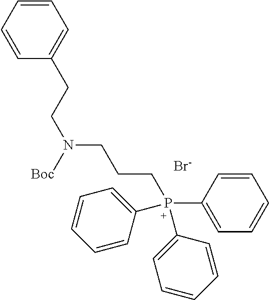

A solution of (3-bromopropyl) triphenylphosphanium bromide (1.00 g, 2.15 mmol, 1.0 eq) and 2-phenylethylamine (0.78 g, 6.46 mmol, 3.0 eq) in EtOH (10 mL) was stirred for 3 h at 78° C. The reslting mixture was allowed to cool down to room temperature, TEA (1.09 g, 10.8 mmol, 5.0 eq) and (Boc) 2O (2.82 g, 12.9 mmol, 6.0 eq) was added, the reaction mixture was stirred for 1 h at room temperature and then concentrated under reduced pressure. The resulting mixture was partitioned between DCM and brine, dried over anhydrous Na₂SO₄ and filtered. The filtrate was concentrated under reduced pressure and the crude residue was purified by silica gel column chromatography (90:10 DCM/MeOH) to afford (3-((tert-butoxycarbonyl)(phenethyl) amino)propyl) triphenylphosphonium bromide 60a (0.58 g, 45%) as a white solid. ¹H NMR (400 MHZ, Chloroform-d) 8 7.94-7.74 (m, 9H), 7.73-7.57 (m, 6H), 7.27-7.16 (m, 4H), 7.15-7.06 (m, 1H), 4.10-3.77 (m, 2H), 3.54 (t, J=5.6 Hz, 4H), 2.84 (q, J=6.5 Hz, 2H), 2.02-1.63 (m, 2H), 1.31 (s, 9H).

### tert-Butyl-((E)-4-((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) but-3-en-1-yl)(phenethyl)carbamate (61a)

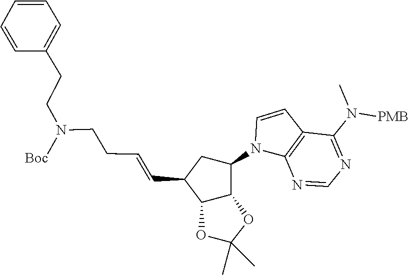

To a stirred solution of (3-((tert-butoxycarbonyl)(phenethyl) amino)propyl) triphenylphosphonium bromide 60 (554 mg, 0.92 mmol, 2.0 eq) in THF (5.0 mL) was added n-BuLi in hexanes (0.37 mL, 0.92 mmol, 2.0 eq) dropwise at −78 oC under N₂. The resulting mixture was stirred for 30 min at −78 oC under N₂ and then (3αR,4S,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxole-4-carbaldehyde 19a (200 mg, 0.46 mmol, 1.0 eq) in THF (2.0 mL) was added, dropwise, at −78 oC.

The reaction mixture was allowed to warm to room temperature and stirred for an additional 1 h. The resulting mixture was quenched by the addition of water (0.5 mL) at room temperature and then concentrated under reduced pressure. The crude residue (500 mg) was purified by silica gel column chromatography (1:1 Petroleum ether/AcOEt) to afford tert-butyl-((E)-4-((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) but-3-en-1-yl)(phenethyl)carbamate 61a (180 mg, 58%) as a yellow semi-solid. LCMS [conditions: XBridge C18, 50*3.0 mm, 3.5 μm, Mobile Phase A: Water/5 mM NH₄HCO₃, Mobile Phase B: Methanol, 10%-95% B-2 min, 1.00 mL/min, ES, m/z]: TR=1.70 min; [M+H]⁺: 682

### (1R,2S,3R,5R)-3-(4-(Methylamino)-7H-pyrrolo[2,3-d]pyrimidin-7-yl)-5-((E)-4-(phenethylamino) but-1-en-1-yl) cyclopentane-1.2-diol (422)

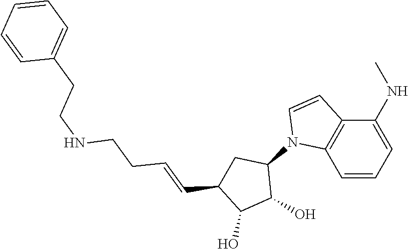 [SMILES: CNc1cccc2c1ccn2[C@@H]1C[C@H](/C=C/CCNCCc2ccccc2)[C@@H](O)[C@H]1O]

To tert-butyl-((E)-4-((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) but-3-en-1-yl)(phenethyl)carbamate 61a (50 mg, 0.07 mmol, 1.0 eq) was added TFA (1.5 mL) and the reaction mixture was stirred for 16 h at room temperature. The resulting mixture was concentrated under reduced pressure and the crude product was purified by Prep-HPLC [Column, XBridge Shield RP18 OBD Column, 19*150 mm, 5 μm; mobile phase, Water (10 mmol/L NH₄HCO₃+0.1% NH₃.H₂O) and ACN (21% ACN up to 39% in 7 min); Detector, UV 220 nm]: to afford (1R,2S,3R,5R)-3-(4-(methylamino)-7H-pyrrolo[2,3-d]pyrimidin-7-yl)-5-((E)-4-(phenethylamino) but-1-en-1-yl) cyclopentane-1,2-diol 293 (13 mg, 42%) as a white solid. ¹H NMR (400 MHZ, DMSO-d₆) δ 8.13 (s, 1H), 7.38 (d, J=5.0 Hz, 1H), 7.30-7.10 (m, 5H), 6.54 (d, J=3.4 Hz, 1H), 5.65-5.31 (m, 2H), 4.83 (d, J=10.7 Hz, 2H), 4.17 (s, 1H), 3.77 (t, J=5.7 Hz, 1H), 2.96 (d, J=4.6 Hz, 3H), 2.89-2.82 (m, 1H), 2.79-2.65 (m, 4H), 2.61-2.53 (m, 1H), 2.25-2.13 (m, 3H), 1.65-1.50 (m, 1H). LCMS [conditions: Halo C18, 50*3.0 mm, 2.7 μm, Mobile Phase A: Water/0.05% TFA, Mobile Phase B: Acetonitrile, 5%-95% B-3 min, 1.50 mL/min, ES, m/z]: TR=0.77 min; [M+H]⁺: 422

### Synthesis of (1R,2S,3R,5S)-3-(4-(methylamino)-7H-pyrrolo[2,3-d]pyrimidin-7-yl)-5-(4-(phenethylamino) butyl) cyclopentane-1,2-diol (423)

### tert-Butyl-(4-((3αR,4S,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) butyl)(phenethyl)carbamate (62a)

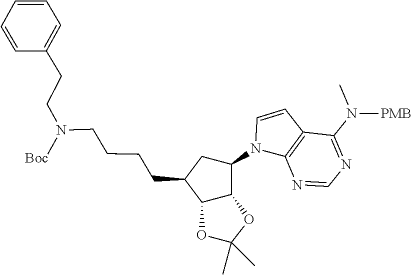

To a solution of tert-butyl-((E)-4-((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) but-3-en-1-yl)(phenethyl)carbamate 61a (55 mg, 0.08 mmol, 1.0 eq) in 10 mL MeOH was added Pd/C(10%, 20 mg) under nitrogen and the reaction mixture was hydrogenated at room temperature for 30 min under a hydrogen balloon. The resulting mixture was filtered through a Celite pad and concentrated under reduced pressure to afford tert-butyl-(4-((3αR,4S,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) butyl)(phenethyl)carbamate 62a (40 mg, 72%) as a yellow semi-solid. LCMS [conditions: XBridge C18, 50*3.0 mm, 3.5 μm, Mobile Phase A: Water/0.05% Ammonia, Mobile Phase B: Acetonitrile, 5%-100% B-2 min, 1.20 mL/min, ES, m/z]: TR=1.61 min; [M+H]⁺: 684

### (1R,2S,3R,5S)-3-(4-(Methylamino)-7H-pyrrolo[2,3-d]pyrimidin-7-yl)-5-(4-(phenethylamino) butyl) cyclopentane-12-diol (47?)

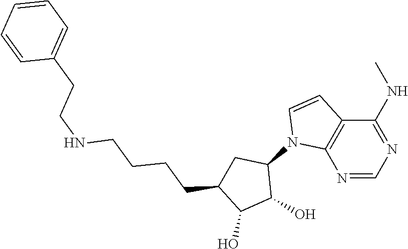 [SMILES: CNc1ncnc2c1ccn2[C@@H]1C[C@H](CCCCNCCc2ccccc2)[C@@H](O)[C@H]1O]

To tert-butyl-(4-((3αR,4S,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) butyl)(phenethyl)carbamate 62a (40 mg, 0.058 mmol, 1.0 eq) was added TFA (1.0 mL) and the reaction mixture was stirred for 16 h at room temperature. The resulting mixture was concentrated under reduced pressure and the crude residue was purified by Prep-HPLC [Column, XBridge Shield RP18 OBD Column, 19*150 mm, 5 μm; mobile phase, ACN and Water (10 mmol/L NH₄HCO₃+0.1% NH₃.H₂O)(16% up to 40% in 7 min); Detector, UV 220 nm]: to afford (1R,2S,3R,5S)-3-(4-(methylamino)-7H-pyrrolo[2,3-d]pyrimidin-7-yl)-5-(4-(phenethylamino) butyl) cyclopentane-1,2-diol 294 (7.0 mg, 22%) as a white solid. ¹H NMR (400 MHz, DMSO-d₆) δ 8.12 (s, 1H), 7.36 (s, 1H), 7.32-7.10 (m, 6H), 6.53 (d, J=3.4 Hz, 1H), 4.95-4.68 (m, 2H), 4.56 (s, 1H), 4.17 (s, 1H), 3.66 (d, J=5.4 Hz, 1H), 2.95 (d, J=4.6 Hz, 4H), 2.80-2.64 (m, 4H), 2.24-2.11 (m, 2H), 1.90-1.79 (m, 1H), 1.47-1.25 (m, 8H). LCMS [conditions: Halo C18, 50*3.0 mm, 2.7 μm, Mobile Phase A: Water/0.05% TFA, Mobile Phase B: Acetonitrile, 5%-95% B-3 min, 1.50 mL/min, ES, m/z]: TR=0.76 min; [M+H]⁺: 424

### Synthesis of (1R,2S,3R,5S)-3-(4-(Methylamino)-7H-pyrrolo[2,3-d]pyrimidin-7-yl)-5-(5-(phenethylamino) pentyl) cyclopentane-1,2-diol (424)

### (4-((tert-Butoxycarbonyl)(phenethyl) amino) butyl) triphenylphosphonium bromide (63a)

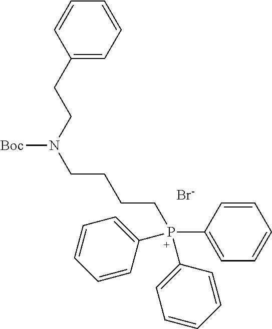

A solution of (4-bromobutyl) triphenylphosphanium bromide (2 q, 4.18 mmol, 1.0 eq) and 2-phenylethylamine (2.03 q, 16.7 mmol, 4.0 eq) in EtOH (20 mL) was stirred for 3 h at 78° C. under N₂. The resulting mixture was allowed to cool down to room temperature and TEA (2.54 q, 25.1 mmol, 6.0 eq) and (Boc) 2O (4.56 q, 20.9 mmol, 5.0 eq) were added and the reaction mixture was stirred for 1 h at room temperature. The resulting mixture was concentrated under reduced pressure and the resulting residue was partitioned between water and DCM. The organic layer were washed with brine (50 mL), dried over anhydrous Na₂SO₄, filtered and the filtrate was concentrated under reduced pressure. The crude residue was purified by silica gel column chromatography (90:10 DCM/MeOH) to afford (4-((tert-butoxycarbonyl)(phenethyl) amino) butyl) triphenylphosphonium bromide 63a (1.3 g, 50%) as a white solid. ¹H NMR (400 MHZ, Chloroform-d) 8 7.99-7.83 (m, 6H), 7.78 (dt, J=7.5, 3.5 Hz, 3H), 7.69 (td, J=7.7, 3.5 Hz, 6H), 7.25 (d, J=7.2 Hz, 2H), 7.21-7.15 (m, 3H), 4.00 (d, J=15.6 Hz, 2H), 3.36 (s, 2H), 3.27-3.16 (m, 2H), 2.78 (t, J=7.5 Hz, 2H), 1.97 (s, 2H), 1.63 (q, J=7.8 Hz, 2H), 1.35 (s, 9H)

### tert-Butyl-((E)-5-((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) pent-4-en-1-yl)(phenethyl)carbamate (64a)

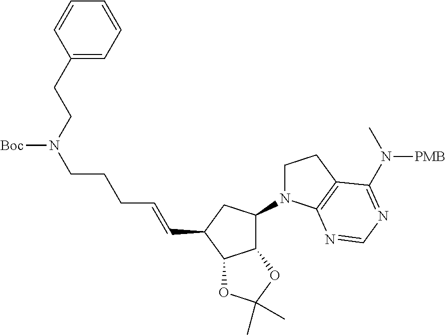

To a stirred solution of (4-((tert-butoxycarbonyl)(phenethyl) amino) butyl) triphenylphosphonium bromide 63a (1.08 q, 1.74 mmol, 2.0 eq) in THF (7 mL) were added n-BuLi in hexanes (0.70 mL, 1.74 mmol, 2.0 eq), dropwise, at −78° C., under N₂. The resulting mixture was stirred for 20 min at −78° C. To this mixture was added (3αR,4S,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxole-4-carbaldehyde 19a (380 mg, 0.87 mmol, 1.0 eq) in THF (3 mL), dropwise, at −78° C. and the reaction mixture was warmed to room temperature stirred for 1 h. The resulting mixture was quenched by the addition of water (0.5 mL) at room temperature and then concentrated under reduced pressure. The residue was purified by silica gel column chromatography (1:1 Petroleum ether/AcOEt) to afford tert-butyl-((E)-5-((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) pent-4-en-1-yl)(phenethyl)carbamate 64a (190 mg, 30%) as a yellow semi-solid. LCMS [conditions: Kinetex XB-C18, 3.0*30 mm, 1.7 μm, Mobile Phase A: Water/0.1% FA, Mobile Phase B: Acetonitrile/0.05% FA, 5%-95% B-1.2 min, 1.50 mL/min, ES, m/z]: TR=0.86 min; [M+H]⁺: 696

### tert-Butyl-(5-((3αR,4S,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) pentyl)(phenethyl)carbamate (65a)

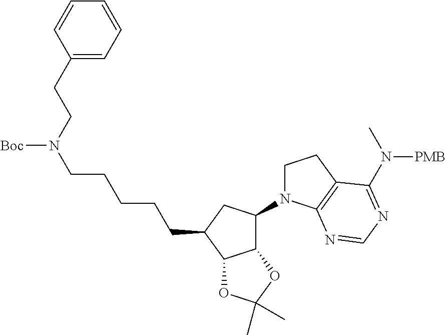

To a solution of tert-butyl-((E)-5-((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) pent-4-en-1-yl)(phenethyl)carbamate 64a (60 mq, 0.086 mmol, 1.0 eq) in 10 mL MeOH was added Pd/C (10%, 20 mq) under nitrogen. The reaction mixture was hydrogenated at room temperature for 30 min under a hydrogen balloon. The resulting mixture was filtered through a Celite pad and concentrated under reduced pressure to afford tert-butyl-(5-((3αR,4S,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) pentyl)(phenethyl)carbamate 65a (55 mg, 91%) as a light yellow semi-solid. LCMS [conditions: Kinetex XB-C18, 3.0*30 mm, 1.7 μm, Mobile Phase A: Water/0.1% FA, Mobile Phase B: Acetonitrile/0.05% FA, 5%-95% B-2 min, 1.50 mL/min, ES, m/z]: TR=1.41 min; [M+H]⁺: 698

### (1R,2S,3R,5S)-3-(4-(Methylamino)-7H-pyrrolo[2,3-d]pyrimidin-7-yl)-5-(5-(phenethylamino) pentyl) cyclopentane-1.2-diol (424)

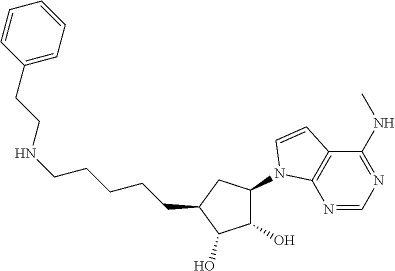 [SMILES: CNc1ncnc2c1ccn2[C@@H]1C[C@H](CCCCCNCCc2ccccc2)[C@@H](O)[C@H]1O]

To tert-butyl-(5-((3αR,4S,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) pentyl)(phenethyl)carbamate 65a (50 mg, 0.07 mmol, 1.0 eq) was added TFA (1.5 mL) and the reaction mixture was stirred for 16 h at room temperature. The resulting mixture was concentrated under reduced pressure and the crude product was purified by Prep-HPLC [Column, XBridge Shield RP18 OBD Column, 19*150 mm, 5 μm; mobile phase, ACN and Water (10 mmol/L NH₄HCO₃+0.1% NH₃.H₂O)(16% up to 40% in 7 min); Detector, UV 220 nm]: to afford (1R,2S,3R,5S)-3-(4-(methylamino)-7H-pyrrolo[2,3-d]pyrimidin-7-yl)-5-(5-(phenethylamino) pentyl) cyclopentane-1,2-diol 424 (17 mg, 53%) as a white solid. ¹H NMR (400 MHZ, DMSO-d₆) δ 8.12 (s, 1H), 7.36 (d, J=5.0 Hz, 1H), 7.31-7.13 (m, 6H), 6.52 (d, J=3.5 Hz, 1H), 4.91-4.71 (m, 2H), 4.56 (s, 1H), 4.27-4.11 (m, 1H), 3.88-3.62 (m, 1H), 2.95 (d, J=4.5 Hz, 3H), 2.76-2.66 (m, 4H), 2.23-2.09 (m, 1H), 1.83 (s, 1H), 1.56 (s, 1H), 1.50-1.19 (m, 8H). LCMS [(conditions: ZORBAX SB-Aq, 50*4.6 mm, 1.8 μm, Mobile Phase A: Water/0.05% TFA, Mobile Phase B: Acetonitrile, 5%-95% B-3 min, 1.50 mL/min, ES, m/z)]: TR=1.23 min; [M+H]⁺: 438. Chiral-HPLC [Column: CHIRALPAK IA-3, 50*4.6 mm, 3 μm IA30CC-UL005, Mobile Phase A: n-Hexane (0.1% DEA), Mobile Phase B: Ethanol; Total Flow: 1.00 mL/min; Conc. of Pump B: 30.0% Oven Temperature: 25° C.]: Two peaks, TR=3.20 min (major peak), 6.66 min (minor peak); dr=95.3:4.7.

### Synthesis of (1R,2S,3R,5R)-3-(4-(methylamino)-7H-pyrrolo[2,3-d]pyrimidin-7-yl)-5-((E)-5-(phenethylamino) pent-1-en-1-yl) cyclopentane-1,2-diol (425)

### (1R,2S,3R,5R)-3-(4-(Methylamino)-7H-pyrrolo[2,3-d]pyrimidin-7-yl)-5-((E)-5-(phenethylamino) pent-1-en-1-yl) cyclopentane-1.2-diol (425)

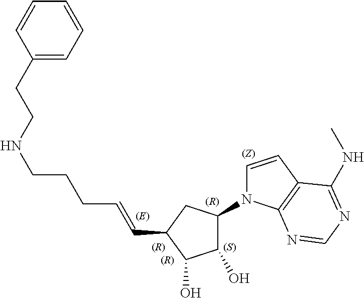

A solution of tert-butyl-((E)-5-((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) pent-4-en-1-yl)(phenethyl)carbamate 64a (20 mg, 0.03 mmol, 1.0 equiv) in TFA (1.0 mL) was stirred for 2 h at rt. The resulting mixture was concentrated under reduced pressure and the crude residue was purified by reverse phase flash chromatography [column, C18 silica gel; mobile phase, ACN in water, 25% to 40% gradient in 10 min; detector, UV 220 nm] to afford (1R,2S,3R,5R)-3-(4-(methylamino)-7H-pyrrolo[2,3-d]pyrimidin-7-yl)-5-((E)-5-(phenethylamino) pent-1-en-1-yl) cyclopentane-1,2-diol 425 (4.2 mg, 36%) as a white solid. ¹H NMR (400 MHZ, DMSO-d₆) δ 8.13 (s, 1H), 7.38 (d, J=4.9 Hz, 1H), 7.30-7.13 (m, 6H), 6.53 (d, J=3.5 Hz, 1H), 5.51-5.31 (m, 2H), 4.89-4.80 (m, 2H), 4.76 (br s, 1H), 4.19 (q, J=6.3 Hz, 1H), 3.76 (t, J=5.7 Hz, 1H), 2.96 (d, J=4.5 Hz, 3H), 2.90-2.78 (m, 1H), 2.74-2.65 (m, 4H), 2.19 (dt, J=12.7, 8.2 Hz, 1H), 2.09 (q, J=7.6 Hz, 2H), 1.57 (dt, J=12.7, 9.9 Hz, 1H), 1.49-1.38 (m, 2H). LCMS [(conditions: HPH-C18, 50*3.0 mm, 2.7 μm, Mobile Phase A: Water/0.05% ammonia water, Mobile Phase B: Acetonitrile, 5%-95% B-5 min, 1.20 mL/min, ES, m/z)]: TR=2.18 min; [M+H]⁺: 436. Chiral-SFC [Column: Solvent B: SC 100×4.6 mm 3.0 μm: MeOH (0.2% MIPA), Oven Temperature: 35° C., Total Flow: 3.00 mL/min, keep the Conc. of Pump B: 50.0% in 5 mins, BPR Pressure: 15.00 MP]: Two diastereomers, TR=3.11 min (major peak), TR=3.27 min (minor isomer); dr=98:2.

### Synthesis of (1R,2S,3R,5R)-3-[4-(methylamino) pyrrolo[2,3-d]pyrimidin-7-yl]-5-{3-[(2-phenylethyl) amino]prop-1-yn-1-yl}cyclopentane-1,2-diol (426)

### 7-((3αS,4R,6R,6αR)-6-Ethynyl-2,2-dimethyltetrahydro-4H-cyclopenta[d][1,3]dioxol-4-yl)-N-(4-methoxybenzyl)-N-methyl-7H-pyrrolo[2,3-d]pyrimidin-4-amine (42a)

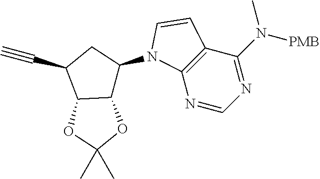

To a stirred solution of (3αR,4S,6R,6αS)-6-(4-{[(4-methoxyphenyl)methyl](methyl) amino}pyrrolo[2,3-d]pyrimidin-7-yl)-2,2-dimethyl-tetrahydro-3aH-cyclopenta[d][1,3]dioxole-4-carbaldehyde 13a (1.00 g, 2.29 mmol, 1.0 eq) and K₂CO₃ (0.63 g, 4.58 mmol, 2.0 eq) in THF (10 mL) and MeOH (10 mL) was added dimethyl(1-diazo-2-oxopropyl) phosphonate (0.88 g, 4.58 mmol, 2.0 eq) dropwise at 10° C. and the reaction mixture was stirred at 10° C. for 1 h. The resulting mixture was concentrated under vacuum and the crude residue was purified by silica gel column chromatography (1/1 Petroleum ether/AcOEt) to afford 7-((3αS,4R,6R,6αR)-6-ethynyl-2,2-dimethyltetrahydro-4H-cyclopenta[d][1,3]dioxol-4-yl)-N-(4-methoxybenzyl)-N-methyl-7H-pyrrolo[2,3-d]pyrimidin-4-amine 42a (220 mg, 22%) as a colourless oil. ¹H NMR (400 MHZ, DMSO-d₆) δ 8.19 (s, 1H), 7.42 (d, J=3.6 Hz, 1H), 7.19 (d, J=8.3 Hz, 2H), 6.93-6.85 (m, 2H), 6.58 (d, J=3.6 Hz, 1H), 5.05 (td, J=6.8, 3.5 Hz, 1H), 4.94 (s, 2H), 4.88 (dd, J=7.2, 4.9 Hz, 1H), 4.75 (t, J=6.8 Hz, 1H), 3.72 (s, 3H), 3.28 (s, 3H), 3.14 (d, J=2.4 Hz, 1H), 2.95-2.85 (m, 1H), 2.48-2.29 (m, 2H), 1.48 (s, 3H), 1.23 (s, 3H).

### 7-((3αS,4R,6R,6αR)-2,2-Dimethyl-6-(3-(phenethylamino)prop-1-yn-1-yl) tetrahydro-4H-cyclopenta[d][1,3]dioxol-4-yl)-N-(4-methoxybenzyl)-N-methyl-7H-pyrrolo[2,3-d]pyrimidin-4-amine (43a)

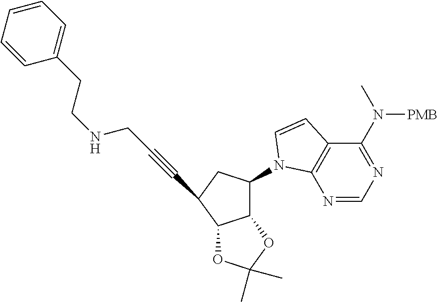

To a stirred solution of 7-((3αS,4R,6R,6αR)-6-ethynyl-2,2-dimethyltetrahydro-4H-cyclopenta[d][1,3]dioxol-4-yl)-N-(4-methoxybenzyl)-N-methyl-7H-pyrrolo[2,3-d]pyrimidin-4-amine 42a (100 mg, 0.23 mmol, 1.0 eq), 2-phenylethylamine (42 mg, 0.35 mmol, 1.5 eq) and CH₂O (15.3 mg, 0.46 mmol, 2.0 eq) in DMSO (5.0 mL) was added Cul (22.0 mg, 0.12 mmol, 0.5 eq) under N₂ and the reaction mixture was stirred at room temperature for 2 h. The resulting mixture was purified directly by reverse flash chromatography [column, C₁₈ silica gel; mobile phase, ACN in water, 10% to 80% gradient in 15 min; detector, UV 220 nm]: to afford 7-((3αS,4R,6R,6αR)-2,2-dimethyl-6-(3-(phenethylamino)prop-1-yn-1-yl) tetrahydro-4H-cyclopenta[d][1,3]dioxol-4-yl)-N-(4-methoxybenzyl)-N-methyl-7H-pyrrolo[2,3-d]pyrimidin-4-amine 43a (55 mg, 42%) as a yellow oil. LCMS: [column: Xbridge shield RP18,50*3.0 mm, 3.5 μm; mobile phase A: water/0.05% ammonia, mobile phase B: ACN, 5%-95%-3 min, ES, m/z]: TR=2.00 min; [M+H]⁺: 566.3.

### 7-[(3αS,4R,6R,6αR)-2,2-Dimethyl-6-{3-[(2-phenylethyl) amino]prop-1-yn-1-yl}-tetrahydro-3aH-cyclopenta[d][1,3]dioxol-4-yl]-N-[(4-methoxyphenyl)methyl]-N-methylpyrrolo[2,3-d]pyrimidin-4-amine (426)

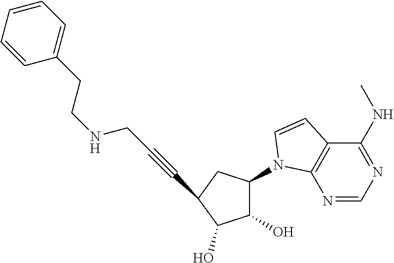 [SMILES: CNc1ncnc2c1ccn2[C@@H]1C[C@H](C#CCNCCc2ccccc2)[C@@H](O)[C@H]1O]

To 7-((3αS,4R,6R,6αR)-2,2-dimethyl-6-(3-(phenethylamino)prop-1-yn-1-yl) tetrahydro-4H-cyclopenta[d][1,3]dioxol-4-yl)-N-(4-methoxybenzyl)-N-methyl-7H-pyrrolo[2,3-d]pyrimidin-4-amine 43a (55 mg, 0.097 mmol) was added TFA (2.0 mL) and the reaction mixture was stirred for 12 h at room temperature. The resulting mixture was concentrated under vacuum and the crude residue was purified by Prep-HPLC [Xbridge Prep C18 OBD column, 5 μm, 19*150 mm; mobile phase, Water (0.03% NH₄OH) and CH₃CN (25% CH₃CN up to 45% in 12 min); Detector, UV 220&254 nm]: to afford 7-[(3αS,4R,6R,6αR)-2,2-dimethyl-6-{3-[(2-phenylethyl) amino]prop-1-yn-1-yl}-tetrahydro-3aH-cyclopenta[d][1,3]dioxol-4-yl]-N-[(4-methoxyphenyl)methyl]-N-methylpyrrolo[2,3-d]pyrimidin-4-amine 426 (10 mg, 25%) as a whitesolid.

¹H NMR (400 MHZ, DMSO-d₆) δ 8.12 (s, 1H), 7.40 (d, J=5.4 Hz, 1H), 7.32-7.13 (m, 6H), 6.56 (d, J=3.4 Hz, 1H), 5.08 (d, J=5.1 Hz, 1H), 4.97 (d, J=6.1 Hz, 1H), 4.85 (q, J=8.1 Hz, 1H), 4.26 (q, J=6.3 Hz, 1H), 3.99 (q, J=5.2 Hz, 1H), 3.37 (d, J=2.1 Hz, 2H), 2.96 (d, J=4.5 Hz, 3H), 2.85-2.77 (m, 2H), 2.75-2.65 (m, 3H), 2.49-2.41 (m, 1H), 1.96-1.70 (m, 2H). LCMS [column: HPH-C18,50*3.0 mm, 2.7 μm; mobile phase A: water/0.05% ammonia water, mobile phase B: ACN, 5%-30%-95% B-6.0 min (+), ES, m/z]: TR=4.20 min (major isomer), 4.33 min (minor isomer); [M+H]⁺: 406.2. Chiral-SFC [Column: Solvent B: SC 100×4.6 mm 3.0 μm: MeOH (0.2% MIPA), Oven Temperature: 35° C., Total Flow: 3.00 mL/min, Start Conc. of Pump B: 10.0%, BPR Pressure: 15.00 MP]: Two diastereomers, TR=2.66 min (major peak), 2.89 min (minor isomer); dr=95:5.

### Synthesis of 2-{[(1R,2R,3S,4R)-2,3-dihydroxy-4-[4-(Methylamino) pyrrolo[2,3-d]pyrimidin-7-yl]cyclopentyl]methoxy}-N-(2-phenylethyl)acetamide (689)

### 2-(((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) methoxy)acetic acid (17h)

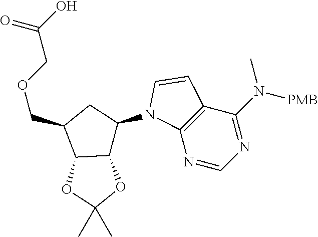

A solution of methyl 2-{[(3αR,4R,6R,6αS)-6-(4-{[(4-methoxyphenyl)methyl](methyl) amino}pyrrolo[2,3-d]pyrimidin-7-yl)-2,2-dimethyl-tetrahydro-3aH-cyclopenta[d][1,3]dioxol-4-yl]methoxy}acetate (80 mg, 0.16 mmol, 1.0 eq) and NaOH (12.5 mg, 0.32 mmol, 2.0 eq) in MeOH (2.0 mL) and H₂O (0.5 mL) was stirred for 2 h at room temperature. The reaction mixture was acidified to pH 2-3 with 1 M HCl. The precipitated solids were collected by filtration and washed with water. The resulting solids was dried by infrared lamp to afford 2-(((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) methoxy)acetic acid 17h (62 mg, 80%) as a white solid. LCMS [column: XBridge C18, 50*3.0 mm, 3.5 μm, Mobile Phase A: Water/5 mM NH₄HCO₃, Mobile Phase B: Methanol, 10%-95% B-3 min (+), 1.50 mL/min]: TR=1.61 min; [M+H]⁺: 497.2.

### 2-(((3αR,4R,6R,6αS)-6-(4-((4-Methoxybenzyl)(methyl) amino)-7H-pyrrolo[2,3-d]pyrimidin-7-yl)-2,2-dimethyltetrahydro-4H-cyclopenta[d][1,3]dioxol-4-yl) methoxy)-N-phenethylacetamide (18h)

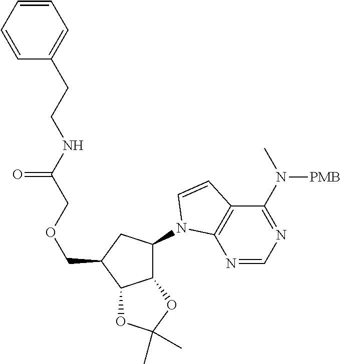

To a stirred solution of 2-(((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) methoxy)acetic acid 17h (60 mg, 0.12 mmol, 1.0 eq) and HATU (68.9 mg, 0.18 mmol, 1.5 eq) in DMF (2 mL) was added DIEA (31.2 mg, 0.24 mmol, 2.0 eq). To this mixture was then added 2-phenylethylamine (17.6 mg, 0.15 mmol, 1.2 eq) in several portions at room temperature. The reaction mixture was stirred for additional 2 h at room temperature, the resulting mixture was evaporated under reduce pressure and purified by silica gel column chromatography (AcOEt) to afford 2-(((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl) methoxy)-N-phenethylacetamide 18h (60 mg, 58%) as a white solid. LCMS [column: XBridge C18, 50*3.0 mm, 3.5 μm, Mobile Phase A: Water/5 mM NH₄HCO₃, Mobile Phase B: Methanol, 10%-95% B-3 min (+), 1.50 mL/min]: TR=2.28 min; [M+H]⁺: 600.4.

### 2-(((1R,2R,3S,4R)-2,3-Dihydroxy-4-(4-(methylamino)-7H-pyrrolo[2,3-d]pyrimidin-7-yl) cyclopentyl) methoxy)-N-phenethylacetamide (689)

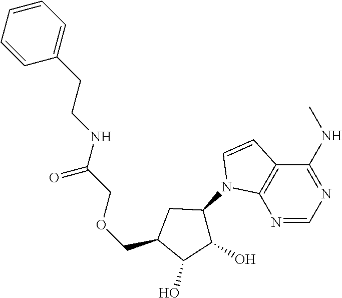 [SMILES: CNc1ncnc2c1ccn2[C@@H]1C[C@H](COCC(=O)NCCc2ccccc2)[C@@H](O)[C@H]1O]

To 2-(((3αR,4R,6R,6αS)-6-(4-((4-Methoxybenzyl)(methyl) amino)-7H-pyrrolo[2,3-d]pyrimidin-7-yl)-2,2-dimethyltetrahydro-4H-cyclopenta[d][1,3]dioxol-4-yl) methoxy)-N-phenethylacetamide 18h (20 mg, 0.033 mmol, 1.0 eq) in TFA (1 mL) was stirred for 2 h at room temperature. The resulting mixture was concentrated under reduced pressure and was purified by Prep-HPLC [(Waters I): Column, Xbridge Prep C18 OBD column, 5 μm, 19*150 mm; mobile phase, Water (0.03% NH₄OH) and CH₃CN (16% CH₃CN up to 34% in 10 min); Detector, UV 220&254 nm]: to afford 2-(((1R,2R,3S,4R)-2,3-dihydroxy-4-(4-(methylamino)-7H-pyrrolo[2,3-d]pyrimidin-7-yl) cyclopentyl) methoxy)-N-phenethylacetamide 689 (4.3 mg, 29%) as a white solid. ¹H NMR (400 MHz, DMSO-d₆) δ 8.13 (s, 1H), 7.69 (br s, 1H), 7.38 (br s, 1H), 7.30-7.11 (m, 6H), 6.54 (d, J=3.5 Hz, 1H), 4.90-4.82 (m, 1H), 4.81 (d, J=6.5 Hz, 1H), 4.70 (d, J=4.6 Hz, 1H), 4.19 (dt, J=8.1, 6.0 Hz, 1H), 3.87 (s, 2H), 3.83 (d, J=4.6 Hz, 1H), 3.54 (dd, J=9.4, 5.5 Hz, 1H), 3.44 (dd, J=9.4, 5.9 Hz, 1H), 3.39-3.33 (m, 1H), 2.96 (d, J=4.6 Hz, 3H), 2.74 (t, J=7.5 Hz, 2H), 2.25-2.11 (m, 2H), 1.64-1.50 (m, 1H). LCMS [column HPH-C18,50*3.0 mm, 2.7 μm. mobile phase A: water/0.05% ammonia water, mobile phase B: ACN, 5%-95% B-3 min (+), 1.20 mL/min]: TR=1.09 min; [M+H]⁺: 440.2. Chiral-HPLC [Column: YMC Cellulose-SC, 100*4.6 mm, 3 μm, 119IA70110; Mobile Phase A: n-Hexane (0.1% DEA), Mobile Phase B: Ethanol; Total Flow: 1.00 mL/min; Conc. of Pump B: 50.0%; Oven Temperature: 25° C.]: TR=2.60 min. dr >99:1.

### Synthesis of (1R,2S,3R,5S)-3-(4-(methylamino)-7H-pyrrolo[2,3-d]pyrimidin-7-yl)-5-(((3-(phenethylamino)propyl) thio) methyl) cyclopentane-1,2-diol (427)

### S-(((3αR,4S,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl)ethanethioate (92a)

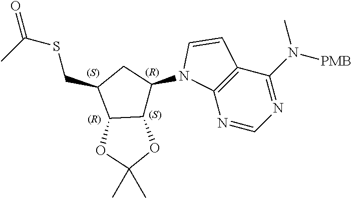

To a solution of PPh₃ (448 mg, 1.71 mmol, 1.5 eq) in THF (5.0 mL) was added DEAD (238 mg, 1.37 mmol, 1.2 eq) over 5 min, followed by a solution of [(3αR,4R,6R,6αS)-6-(4-{[(4-methoxyphenyl)methyl](methyl) amino}pyrrolo[2,3-d]pyrimidin-7-yl)-2,2-dimethyl-tetrahydro-3aH-cyclopenta[d][1,3]dioxol-4-yl]methanol (Intermediate A, 500 mg, 1.14 mmol, 1.0 eq) in THF (5.0 mL), dropwise at 0° C. To the resultant mixture was then added ethanethioic S-acid (122 mg, 1.60 mmol, 1.4 eq) dropwise over 5 min and the reaction mixture was stirred for 1 h at, 0° C., under nitrogen. The resulting mixture was concentrated under vacuum and purified by silica gel column chromatography (4:1 Petroleum ether/AcOEt) to afford S-(((3αR,4S,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl)ethanethioate 92a (550 mg, crude) as a yellow crude oil. LCMS [conditions: Halo C18, 30*3.0 mm, 2.0 μm, Mobile Phase A: Water/5 mM TFA, Mobile Phase B: Acetonitrile, 5%-100% B-1.2 min (+), 1.50 mL/min, ES, m/z]: TR=0.64 min; [M+H]⁺: 497.

### tert-Butyl-(3-((((3αR,4S,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl) thio)propyl)(phenethyl)carbamate (93a)

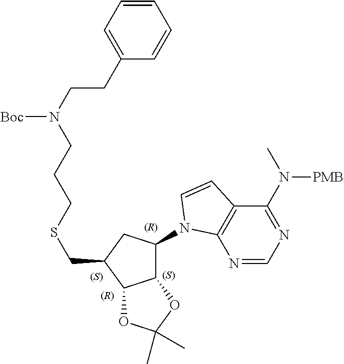

To S-(((3αR,4S,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl)ethanethioate 92a (300 mg, 0.60 mmol, 1.0 eq) in MeOH (5.0 mL) was added tert-butyl-(3-bromopropyl)(phenethyl)carbamate 58a (248 mg, 0.73 mmol, 1.2 eq) and K₂CO₃ (125 mg, 0.91 mmol, 1.5 eq) and the reaction mixture was stirred for 2 h at room temperature under a nitrogen. The resulting mixture was filtered and the filtrate was concentrated under vacuum. The crude residue was purified by silica gel column chromatography (3:1 Petroleum ether/AcOEt) to afford tert-butyl-(3-((((3αR,4S,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl) thio)propyl)(phenethyl)carbamate 93a (170 mg, 39%) as a light yellow oil. LCMS [conditions: Halo C18, 30*3.0 mm, 2.0 μm, Mobile Phase A: Water/5 mM TFA, Mobile Phase B: Acetonitrile, 5%-100% B-1.2 min (+), 1.50 mL/min, ES, m/z]: TR=0.80 min; [M+H]⁺: 716.

### (1R,2S,3R,5S)-3-(4-(Methylamino)-7H-pyrrolo[2,3-d]pyrimidin-7-yl)-5-(((3-(phenethylamino)propyl) thio) methyl) cyclopentane-1,2-diol (427)

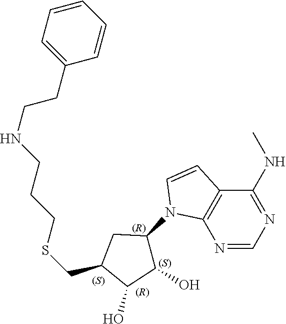

To tert-butyl-(3-((((3αR,45,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl) thio)propyl)(phenethyl)carbamate 93a (50 mg, 0.07 mmol, 1.0 eq) was added TFA (0.5 mL, 6.73 mmol) and the reaction mixture was stirred overnight at room temperature under nitrogen. The resulting mixture was concentrated and the crude residue was purified by Prep-HPLC [Column, Xselect CSH OBD Column 30×150 mm 5 μm; mobile phase, water (10MMOL/L NH₄HCO₃) and ACN (10% Phase B up to 50% in 8 min); Detector, UV 220 nm]: to afford (1R,2S,3R,5S)-3-(4-(methylamino)-7H-pyrrolo[2,3-d]pyrimidin-7-yl)-5-(((3-(phenethylamino)propyl) thio) methyl) cyclopentane-1,2-diol 427 (5.0 mg, 15%) as a white solid. 1H NMR (400 MHZ, DMSO-d₆) δ 8.12 (s, 1H), 7.37 (d, J=5.6 Hz, 1H), 7.27 (t, J=7.4 Hz, 2H), 7.26-7.13 (m, 4H), 6.53 (d, J=3.5 Hz, 1H), 4.84 (dd, J=13.5, 6.9 Hz, 2H), 4.74-4.68 (m, 1H), 4.23 (q, J=6.5 Hz, 1H), 3.79 (d, J=5.1 Hz, 1H), 2.96 (d, J=4.5 Hz, 3H), 2.76-2.69 (m, 5H), 2.62 (t, J=7.0 Hz, 2H), 2.61-2.57 (m, 2H), 2.26-2.24 (m, 1H), 2.09 (s, 1H), 1.67 (q, J=7.2 Hz, 2H), 1.55 (q,)=10.5 Hz, 1H). LCMS [conditions: ZORBAX SB-Aq, 50*4.6 mm, 1.8 μm, Mobile Phase A: Water/0.05% TFA, Mobile Phase B: Acetonitrile, 5%-95% B-3 min, 1.50 mL/min, ES, m/z]: TR=1.20 min; [M+H]⁺: 456. CHIRAL_HPLC [Column: YMC Cellulose-SB, 100*4.6 mm, 3 μm. Phase A: Ethanol (0.1% DEA). Phase B: Methanol, keep 50.0% Phase B in 10 min]: TR=1.75 min; single peak.

### Synthesis of (1R,2S,3R,5S)-3-(4-(methylamino)-7H-pyrrolo[2,3-d]pyrimidin-7-yl)-5-(((3-(phenethylamino)propyl) sulfinyl)methyl) cyclopentane-1,2-diol (428)

### tert-Butyl-(3-((((3αR,4S,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl) thio)propyl)(phenethyl) carhamate (94a)

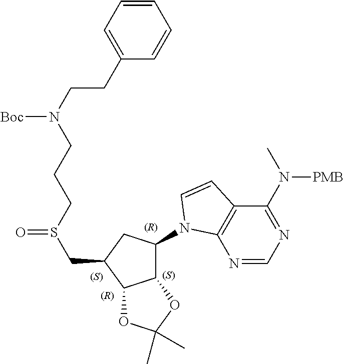

To a solution of tert-butyl-(3-((((3αR,45,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl) thio)propyl)(phenethyl)carbamate 93a (200 mg, 0.28 mmol, 1.0 eq) in DCM (5.0 mL) was added m-CPBA (50 mg, 0.29 mmol, 1.05 eq) in several portions at room temperature and the resulting mixture was stirred for 2 h at room temperature. The resulting mixture was concentrated under vacuum and the crude residue was purified by silica gel column chromatography (2:1 Petroleum ether/AcOEt) to afford tert-butyl-(3-((((3αR,4S,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl) sulfinyl)propyl)(phenethyl)carbamate 94a (150 mg, 73%) as off white solid. LCMS [conditions: Halo C18, 30*3.0 mm, 2.0 μm, Mobile Phase A: Water/5 mM TFA, Mobile Phase B: Acetonitrile, 5%-100% B-1.2 min (+), 1.50 mL/min, ES, m/z]: TR=0.95 min; [M+H]⁺: 732.

### (1R,2S,3R,5S)-3-(4-(Methylamino)-7H-pyrrolo[2,3-d]pyrimidin-7-yl)-5-(((3-(phenethylamino)propyl) sulfinyl)methyl) cyclopentane-1,2-diol (428)

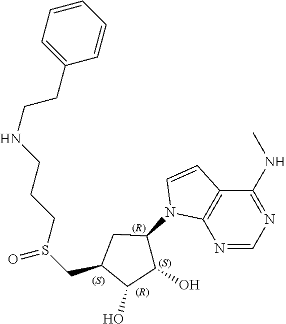

To tert-butyl-(3-((((3αR,4S,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl) sulfinyl)propyl)(phenethyl)carbamate 94a (80 mg, 0.11 mmol, 1.0 eq) was added TFA (2.0 mL) at room temperature and the reaction mixture was stirred overnight at room temperature. The resulting mixture was concentrated under vacuum and the crude residue was purified by Prep-HPLC [Column: Xselect CSH OBD Column 30×150 mm 5 μm; Mobile Phase A: water (0.05% NH₃.H₂O), Mobile Phase B: ACN; Flow rate: 60 mL/min; Gradient: 25% B to 63% B in 8 min, 63% B; Wave Length: 220 nm; TR (min): 7]: to afford (1R,2S,3R,5S)-3-(4-(methylamino)-7H-pyrrolo[2,3-d]pyrimidin-7-yl)-5-(((3-(phenethylamino)propyl) sulfinyl)methyl) cyclopentane-1,2-diol 428 (25.8 mg, 50%) as an off-white solid. ¹H NMR (400 MHZ, DMSO-d₆) δ 8.13 (s, 1H), 7.40 (br s, 1H), 7.31-7.13 (m, 6H), 6.54 (d, J=4.2 Hz, 1H), 4.90 (d, J=6.4 Hz, 2H), 4.84 (d, J=8.1 Hz, 1H), 4.23 (s, 1H), 3.90 (s, 1H), 3.10-2.96 (m, 4H), 2.86-2.77 (m, 2.4H), 2.77-2.69 (m, 4H), 2.68-2.66 (m, 3H), 2.36 (q, J=7.9, 7.0 Hz, 2H), 1.78 (t, J=7.3 Hz, 2H), 1.73-1.65 (m, 1H). LCMS [conditions: XBridge C18, 50*3.0 mm, 3.5 μm, Mobile Phase A: Water/5 mM NH₄HCO₃, Mobile Phase B: Methanol, 10%-95% B-2 min (+), 1.50 mL/min, ES, m/z]: TR=0.69 min; [M+H]⁺: 472. CHIRAL_SFC [Column: SC 100×4.6 mm 3.0 μm. 50 dergree, Solvent B: MeOH (20 mM NH₃), Solvent A: CO₂, keep 30% solvent B for 4 mins]: TR=2.72 min (minor peak), 3.13 min (major peak), dr=32:67.

### Synthesis of (1R,2S,3R,5S)-3-(4-(methylamino)-7H-pyrrolo[2,3-d]pyrimidin-7-yl)-5-(((3-(phenethylamino)propyl) sulfonyl)methyl) cyclopentane-1,2-diol (429)

### tert-Butyl-(3-((((3αR,4S,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl) sulfonyl)propyl)(phenethyl)carbamate (95a)

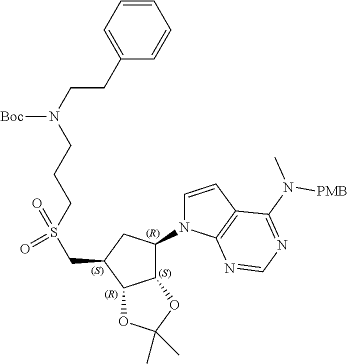

To a solution of tert-butyl (3-((((3αR,4S,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl) thio)propyl)(phenethyl)carbamate 93a (100 mg, 0.14 mmol, 1.0 eq) in DCM (3.0 mL) was added m-CPBA (60 mg, 0.35 mmol, 2.5 eq) in several portions at room temperature and the reaction mixture was stirred for 2 h at room temperature. The resultant mixture was concentrated under vacuum and the crude residue was purified by silica gel column chromatography (1:1 Petroleum ether/AcOEt) to afford tert-butyl-N-(3-{[(3αR,4S,6R,6αS)-6-(4-{[(4-methoxyphenyl)methyl](methyl) amino}pyrrolo[2,3-d]pyrimidin-7-yl)-2,2-dimethyl-tetrahydro-3aH-cyclopenta[d][1,3]dioxol-4-yl]methanesulfonyl}propyl)-N-(2-phenylethyl)carbamate 95a (65 mg, 62%) as off-white solid. LCMS [conditions: Halo C18, 30*3.0 mm, 2.0 μm, Mobile Phase A: Water/5 mM TFA, Mobile Phase B: Acetonitrile, 5%-100% B-1.2 min (+), 1.50 mL/min, ES, m/z]: TR=0.90; [M+H]⁺: 748.

### (1R,2S,3R,5S)-3-(4-(Methylamino)-7H-pyrrolo[2,3-d]pyrimidin-7-yl)-5-(((3-(phenethylamino)propyl) sulfonyl)methyl) cyclopentane-1,2-diol (429)

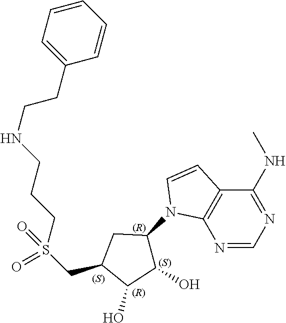

To tert-butyl-(3-((((3αR,4S,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl) sulfonyl)propyl)(phenethyl)carbamate 95a (65 mg, 0.087 mmol, 1.0 eq) was added TFA (2.0 mL) at room temperature and the reaction mixture was stirred overnight at room temperature. The resulting mixture was concentrated under vacuum and the crude residue was purified by Prep-HPLC [Column: Xselect CSH OBD Column 30×150 mm 5 μm; Mobile Phase A: water (0.05% NH₃.H₂O), Mobile Phase B: ACN; Flow rate: 60 mL/min; Gradient: 33% B to 60% B in 8 min, 60% B; Wave Length: 220 nm]: to afford (1R,2S,3R,5S)-3-(4-(methylamino)-7H-pyrrolo[2,3-d]pyrimidin-7-yl)-5-(((3-(phenethylamino)propyl) sulfonyl)methyl) cyclopentane-1,2-diol 429 (19 mg, 45%) as an off-white solid. ¹H NMR (400 MHZ, DMSO-d₆) δ 8.14 (s, 1H), 7.40 (q, J=4.5 Hz, 1H), 7.31-7.13 (m, 6H), 6.54 (d, J=3.5 Hz, 1H), 4.94 (t, J=6.0 Hz, 2H), 4.88-4.77 (m, 1H), 4.18 (q, J=5.8 Hz, 1H), 3.89 (d, J=5.5 Hz, 1H), 3.41 (dd, J=14.0, 3.7 Hz, 1H), 3.18-3.10 (m, 2H), 2.96 (d, J=4.6 Hz, 3H), 2.70-2.65 (m, 4H), 2.63 (t, J=6.7 Hz, 2H), 2.43-2.40 (m, 2H), 1.87-1.64 (m, 3H). LCMS [conditions: XBridge C18, 50*3.0 mm, 3.5 μm, Mobile Phase A: Water/5 mM NH₄HCO₃, Mobile Phase B: Methanol, 10%-95% B-2 min (+), 1.50 mL/min, ES, m/z]: TR=1.13 min; [M+H]⁺: 488. CHIRAL_SFC [Column: Lux-4 100×4.6 mm 3.0 μm. 50 dergree, Solvent B: MeOH (20 mM NH₃), Solvent A: CO₂, keep 50% solvent B for 4 mins]: TR=1.58 min, single peak.

### Synthesis of (1S,2R,3S,5R)-3-(((2-Aminoethyl) thio) methyl)-5-(4-(methylamino)-7H-pyrrolo[2,3-d]pyrimidin-7-yl) cyclopentane-1,2-diol (430)

### 7-((3αS,4R,6S,6αR)-6-(((2-Aminoethyl) thio) methyl)-2,2-dimethyltetrahydro-4H-cyclopenta[d][1,3]dioxol-4-yl)-N-(4-methoxybenzyl)-N-methyl-7H-pyrrolo[2,3-d]pyrimidin-4-amine (96a)

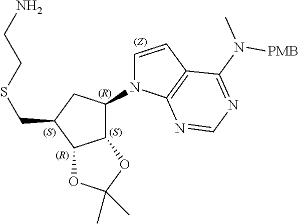

To a mixture of 2-bromoethanamine (599 mg, 4.83 mmol, 3.0 eq) and K₂CO₃ (1.11 g, 8.06 mmol, 5.0 eq) in MeOH (5.0 mL) was added S-(((3αR,4S,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl)ethanethioate 92a (800 mg, 1.61 mmol, 1.0 eq) in MeOH (5.0 mL) dropwise, at room temperature, under nitrogen and the reaction mixture was stirred overnight at room temperature. The resultant mixture was concentrated under vacuum and the crude residue was purified by silica gel column chromatography (1:2 Petroleum ether/AcOEt) to afford 7-((3αS,4R,6S,6αR)-6-(((2-aminoethyl) thio) methyl)-2,2-dimethyltetrahydro-4H-cyclopenta[d][1,3]dioxol-4-yl)-N-(4-methoxybenzyl)-N-methyl-7H-pyrrolo[2,3-d]pyrimidin-4-amine 96a (410 mg, 51%) as a brown solid. ¹H NMR (400 MHZ, Chloroform-d) 8 8.36 (s, 1H), 7.28-7.26 (m, 2H), 6.93 (d, J=3.6 Hz, 1H), 6.91-6.85 (m, 2H), 6.47 (d, J=3.6 Hz, 1H), 5.00-4.93 (m, 4H), 4.62-4.56 (m, 1H), 3.81 (s, 3H), 3.32 (s, 3H), 2.98-2.85 (m, 2H), 2.83-2.69 (m, 2H), 2.54-2.48 (m, 1H), 2.45-2.39 (m, 1H), 2.26-2.15 (m, 1H), 1.59 (s, 3H), 1.33 (s, 3H).

### (1S,2R,3S,5R)-3-(((2-Aminoethyl) thio) methyl)-5-(4-(methylamino)-7H-pyrrolo[2,3-d]pyrimidin-7-yl) cyclopentane-1,2-diol (430)

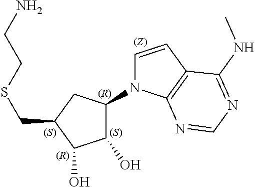

To 7-((3αS,4R,6S,6αR)-6-(((2-aminoethyl) thio) methyl)-2,2-dimethyltetrahydro-4H-cyclopenta[d][1,3]dioxol-4-yl)-N-(4-methoxybenzyl)-N-methyl-7H-pyrrolo[2,3-d]pyrimidin-4-amine 96a (50 mg, 0.10 mmol, 1.0 eq) was added TFA (2.0 mL) at room temperature and the reaction mixture was stirred overnight at room temperature under nitrogen. The resulting mixture was concentrated under vacuum and the crude residue was purified by Prep-HPLC [Column: Xselect CSH OBD Column 30×150 mm 5 μm; Mobile Phase A: water (0.05% NH₃.H₂O), Mobile Phase B: ACN; Flow rate: 60 mL/min; Gradient: 13% B to 43% B in 8 min, 43% B; Wave Length: 220 nm]: to afford (1S,2R,3S,5R)-3-(((2-aminoethyl) thio) methyl)-5-(4-(methylamino)-7H-pyrrolo[2,3-d]pyrimidin-7-yl) cyclopentane-1,2-diol 430 (14.3 mg, 42%) as off-white solid. ¹H NMR (400 MHZ, DMSO-d₆) δ 8.12 (s, 1H), 7.37 (q, J=4.9 Hz, 1H), 7.23 (d, J=3.5 Hz, 1H), 6.53 (d, J=3.5 Hz, 1H), 4.83 (dt, J=10.1, 8.0 Hz, 2H), 4.23 (t, J=6.8 Hz, 1H), 3.79 (t, J=5.0 Hz, 1H), 2.96 (d, J=4.9 Hz, 3H), 2.80 (dd, J=12.7, 6.3 Hz, 1H), 2.69 (t, J=6.8 Hz, 1H), 2.59 (dd, J=12.8, 8.5 Hz, 1H), 2.54-2.50 (m, 4H), 2.28 (dt, J=12.8, 8.2 Hz, 1H), 2.13-2.03 (m, 1H), 1.55 (dt, J=12.5, 9.5 Hz, 1H). LCMS [conditions: XBridge C18, 50*3.0 mm, 3.5 μm, Mobile Phase A: Water/5 mM NH₄HCO₃, Mobile Phase B: Methanol, 10%-95% B-2 min (+), 1.50 mL/min, ES, m/z]: TR=0.92 min; [M+H]⁺: 338. CHIRAL_SFC, Column: OJ 100×4.6 mm 3.0 μm. 50 dergree, Solvent B: MeOH (20 mM NH₃), Solvent A: CO₂, keep 30% solvent B for 4 mins]: TR=0.65 min, single peak.

### Synthesis of (1R,2S,3R,5S)-3-(4-(methylamino)-7H-pyrrolo[2,3-d]pyrimidin-7-yl)-5-(((2-(phenethylamino) ethyl) thio) methyl) cyclopentane-1,2-diol (431)

### 7-((3αS,4R,6S,6αR)-2,2-Dimethyl-6-(((2-(phenethylamino) ethyl) thio) methyl)tetrahydro-4H-cyclopenta[d][1,3]dioxol-4-yl)-N-(4-methoxybenzyl)-N-methyl-7H-pyrrolo[2,3-d]pyrimidin-4-amine (97a)

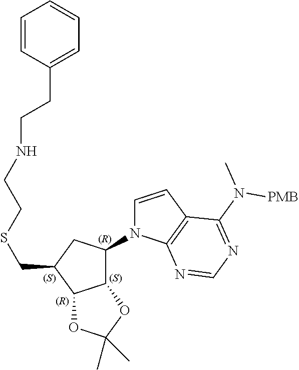

To a stirred solution of 7-((3αS,4R,6S,6αR)-6-(((2-aminoethyl) thio) methyl)-2,2-dimethyltetrahydro-4H-cyclopenta[d][1,3]dioxol-4-yl)-N-(4-methoxybenzyl)-N-methyl-7H-pyrrolo[2,3-d]pyrimidin-4-amine 96a (400 mg, 0.80 mmol, 1.0 eq) in CH₃CN (4.0 mL) was added (2-bromoethyl)benzene (148 mg, 0.80 mmol, 1.0 eq) and K₂CO₃ (222 mg, 1.60 mmol, 2.0 eq) at room temperature. The reaction mixture was stirred for 12 h at 80° C. and the resulting mixture was concentrated under reduced pressure. The crude residue was purified by silica gel column chromatography (1:12 Petroleum ether/AcOEt) to afford 7-((3αS,4R,65,6αR)-2,2-dimethyl-6-(((2-(phenethylamino) ethyl) thio) methyl)tetrahydro-4H-cyclopenta[d][1,3]dioxol-4-yl)-N-(4-methoxybenzyl)-N-methyl-7H-pyrrolo[2,3-d]pyrimidin-4-amine 97a (200 mg, 41%) as a light yellow solid. LCMS [conditions: XBridge C18, 50*3.0 mm, 3.5 μm, Mobile Phase A: Water/5 mM NH₄HCO₃, Mobile Phase B: Methanol, 10%-95% B-2 min (+), 1.50 mL/min, ES, m/z]: TR=1.43 min; [M+H]⁺: 602.4.

### (1R,2S,3R,5S)-3-(4-(Methylamino)-7H-pyrrolo[2,3-d]pyrimidin-7-yl)-5-(((2-(phenethylamino) ethyl) thio) methyl) cyclopentane-1,2-diol (431)

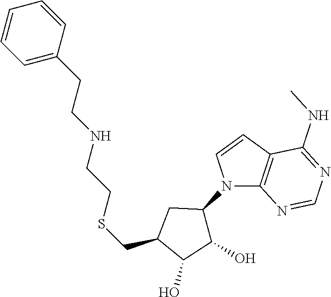 [SMILES: CNc1ncnc2c1ccn2[C@@H]1C[C@H](CSCCNCCc2ccccc2)[C@@H](O)[C@H]1O]

To a solution of 7-((3αS,4R,6S,6αR)-2,2-dimethyl-6-(((2-(phenethylamino) ethyl) thio) methyl)tetrahydro-4H-cyclopenta[d][1,3]dioxol-4-yl)-N-(4-methoxybenzyl)-N-methyl-7H-pyrrolo[2,3-d]pyrimidin-4-amine 97a (0.03 mg, 0.05 mmol, 1.0 eq) in DCM (1.0 mL), at room temperature, was added TFA (1.0 mL) and the reaction mixture was stirred for 16 h. The resulting mixture was concentrated under reduced pressure and the residue was dissolved in water (2.0 mL). The pH value of the solution was adjusted to 8-9 with NH₃.H₂O and the mixture was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 24% B to 49% B over 7 min, 49% B; Wave Length: 220 nm]: to afford (1R,2S,3R,5S)-3-(4-(methylamino)-7H-pyrrolo[2,3-d]pyrimidin-7-yl)-5-(((2-(phenethylamino) ethyl) thio) methyl) cyclopentane-1,2-diol 431 (5.1 mg, 23%) as an off-white solid. ¹H NMR (400 MHZ, DMSO-d₆) δ8.12 (s, 1H), 7.42-7.34 (m, 1H), 7.30-7.12 (m, 6H), 6.53 (d, J=3.5 Hz, 1H), 4.88-4.78 (m, 2H), 4.77-4.68 (m, 1H), 4.29-4.17 (m, 1H), 3.82-3.72 (m, 1H), 2.96 (d, J=4.7 Hz, 3H), 2.84-2.66 (m, 7H), 2.65-2.54 (m, 3H), 2.37-2.19 (m, 1H), 2.14-2.01 (m, 1H), 1.61-1.46 (m, 1H). LCMS [conditions ZORBAX SB-Aq, 50*4.6 mm, 1.8 μm, Mobile Phase A: Water/0.05% TFA, Mobile Phase B: Acetonitrile, 5%-95% B-3 min, 1.50 L/min, ES, m/z]: TR=1.18 min; [M+H]⁺: 442. CHIRAL_SFC, Column: SC 100×4.6 mm 3.0 μm. 50 dergree, Solvent B: MeOH (20 mM NH₃), Solvent A: CO₂, keep 50% solvent B for 4 mins]: TR=1.41 min, single peak.

### Synthesis of (1R,2S,3R,5S)-3-(4-(Methylamino)-7H-pyrrolo[2,3-d]pyrimidin-7-yl)-5-(((2-(phenethylamino) ethyl) sulfinyl)methyl) cyclopentane-1,2-diol (432)

### 7-((3αS,4R,6S,6αR)-2,2-Dimethyl-6-(((2-(phenethylamino) ethyl) sulfinyl)methyl)tetrahydro-4H-cyclopenta[d][1,3]dioxol-4-yl)-N-(4-methoxybenzyl)-N-methyl-7H-pyrrolo[2,3-d]pyrimidin-4-amine (98a)

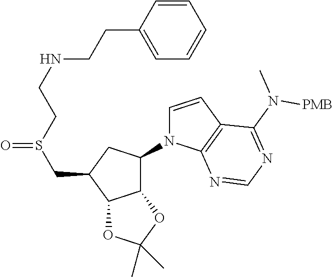

To a solution of 7-((3αS,4R,6S,6αR)-2,2-dimethyl-6-(((2-(phenethylamino) ethyl) thio) methyl)tetrahydro-4H-cyclopenta[d][1,3]dioxol-4-yl)-N-(4-methoxybenzyl)-N-methyl-7H-pyrrolo[2,3-d]pyrimidin-4-amine 97a (0.09 g, 0.20 mmol, 1.0 eq) in DCM (2.0 mL) was added m-CPBA (0.03 g, 0.20 mmol, 1.0 eq) ad the reaction mixture was stirred for 8 h at room temperature. The resulting mixture was concentrated under vacuum and purified by silica gel column chromatography (95:5 dichloromethane:methanol) to afford 7-((3αS,4R,6S,6αR)-2,2-dimethyl-6-(((2-(phenethylamino) ethyl) sulfinyl)methyl)tetrahydro-4H-cyclopenta[d][1,3]dioxol-4-yl)-N-(4-methoxybenzyl)-N-methyl-7H-pyrrolo[2,3-d]pyrimidin-4-amine (98a)(50 mg, 54%) as a colorless oil. LCMS [conditions conditions Xbridge Shield C18, 50*3.0 mm, 3.5 μm, Mobile Phase A: Water/0.05% Ammonia, Mobile Phase B: Acetonitrile, 5%-100% B-2 min (+), 1.50 L/min, ES, m/z]: TR=1.20 min; [M+H]⁺: 618.

### (1R,2S,3R,5S)-3-(4-(Methylamino)-7H-pyrrolo[2,3-d]pyrimidin-7-yl)-5-(((2-(phenethylamino) ethyl) sulfinyl)methyl) cyclopentane-1,2-diol (432)

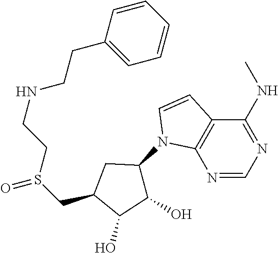 [SMILES: CNc1ncnc2c1ccn2[C@@H]1C[C@H](CS(=O)CCNCCc2ccccc2)[C@@H](O)[C@H]1O]

To a solution of 7-((3αS,4R,6S,6αR)-2,2-dimethyl-6-(((2-(phenethylamino) ethyl) sulfinyl) methyl)tetrahydro-4H-cyclopenta[d][1,3]dioxol-4-yl)-N-(4-methoxybenzyl)-N-methyl-7H-pyrrolo[2,3-d]pyrimidin-4-amine 98a (45 mg, 0.07 mmol, 1.0 eq) in dichloromethane (2.0 mL) was added TFA (2.0 mL) and the reaction mixture was stirred for 16 h at room temperature. The resulting mixture was concentrated under reduced pressure and the residue was basified to pH 10 with NH₃.H₂O. The crude residue was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 ml/min; Gradient: 24% B to 49% B in 7 min, 49% B; Wave Length: 220 nm]: to afford 1R,2S,3R,5S)-3-(4-(methylamino)-7H-pyrrolo[2,3-d]pyrimidin-7-yl)-5-(((2-(phenethylamino) ethyl) sulfinyl)methyl) cyclopentane-1,2-diol 432 (9.8 mg, 30%) as an off-white solid. ¹H NMR (400 MHZ, DMSO-d₆) δ8.13 (s, 1H), 7.44-7.35 (m, 1H), 7.32-7.11 (m, 6H), 6.56-6.50 (m, 1H), 4.94-4.77 (m, 3H), 4.27-4.14 (m, 1H), 3.93-3.82 (m, 1H), 3.10-2.97 (1H), 2.96 (d, J=4.7 Hz, 3H), 2.94-2.88 (m, 2H), 2.87-2.81 (m, 1H), 2.79-2.73 (m, 2H), 2.72-2.65 (m, 2H), 2.40-2.29 (m, 2H), 1.74-1.60 (m, 1H). LCMS [conditions Halo C18, 50*3.0 mm, 2.7 μm, Mobile Phase A: Water/0.05% TFA, Mobile Phase B: Acetonitrile, 5%-95% B-3 min, 1.50 L/min, ES, m/z]: TR=0.66 min; [M+H]⁺: 458 CHIRAL_SFC [Column: SC 100×4.6 mm 3.0 μm. 50° C., Solvent B: MeOH (20 mM NH₃), Solvent A: CO₂, keep Gradient 50% solvent B for 4 mins, two diastereomers]: TR=2.00 min, TR=2.95, dr=1:1.

### Synthesis of (1R,2S,3R,5S)-3-(4-(Methylamino)-7H-pyrrolo[2,3-d]pyrimidin-7-yl)-5-(((2-(phenethylamino) ethyl) sulfonyl)methyl) cyclopentane-1,2-diol (433)

### (1R,2S,3R,5S)-3-(4-(Methylamino)-7H-pyrrolo[2,3-d]pyrimidin-7-yl)-5-(((2-(phenethylamino) ethyl) sulfonyl)methyl) cyclopentane-1,2-diol (433)

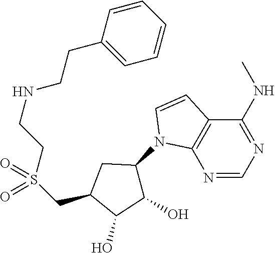 [SMILES: CNc1ncnc2c1ccn2[C@@H]1C[C@H](CS(=O)(=O)CCNCCc2ccccc2)[C@@H](O)[C@H]1O]

To a stirred solution of (1R,2S,3R,5S)-3-(4-(methylamino)-7H-pyrrolo[2,3-d]pyrimidin-7-yl)-5-(((2-(phenethylamino) ethyl) thio) methyl) cyclopentane-1,2-diol 291 (40 mg, 0.10 mmol, 1.0 eq) in DCM (1.0 mL) was added m-CPBA (39 mg, 0.23 mmol, 2.5 eq) in several portions at room temperature. The reaction mixture was stirred for 0.5 h at room temperature and then concentrated under vacuum. The crude residue was purified by Prep-HPLC [Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 30 ml/min; Gradient: 25% B to 53% B in 8 min, 53% B; Wave Length: 220 nm]: to afford (1R,2S,3R,5S)-3-[4-(methylamino) pyrrolo[2,3-d]pyrimidin-7-yl]-5-({2-[(2-phenylethyl)amino]ethanesulfonyl}methyl) cyclopentane-1,2-diol 433 (7.4 mg, 17%) as a white solid. ¹H NMR (400 MHZ, Chloroform-d+D₂O) 8 8.31 (s, 1H), 7.35-7.27 (m, 2H), 7.25-7.19 (m, 3H), 6.95 (d, J=3.7 Hz, 1H), 6.38 (s, 1H), 4.67-4.57 (m, 1H), 4.23 (t, J=7.0 Hz, 1H), 4.01 (t, J=6.8 Hz, 1H), 3.60 (dd, J=14.0, 5.0 Hz, 1H), 3.33-3.06 (m, 8H), 2.98-2.89 (m, 2H), 2.86-2.75 (m, 3H), 2.73-2.68 (m, 1H), 1.95 (q, J=11.6 Hz, 1H). LCMS [conditions Halo C18, 50*3.0 mm, 2.7 μm, Mobile Phase A: Water/0.05% TFA, Mobile Phase B: Acetonitrile, 5%-95% B-3 min-1.5.lcm, 1.50 L/min, ES, m/z]: TR=1.08 min; [M+H]⁺: 474. CHIRAL_SFC [Column: SC 100×4.6 mm 3.0 μm. 50° C., Solvent B: MeOH (20 mM NH₃), Solvent A: CO₂, keep Gradient 30% solvent B for 4 mins]: TR=2.41 min, single peak.

### Synthesis of 3-((((1R,2R,35,4R)-2,3-dihydroxy-4-(4-(methylamino)-7H-pyrrolo[2,3-d]pyrimidin-7-yl) cyclopentyl)methyl) amino)-N-phenethylpropanamide (434)

### tert-Butyl-N-{2-[(2-phenylethyl)carbamoyl]ethyl}carbamate (39a)

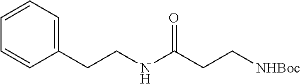 [SMILES: CC(C)(C)OC(=O)NCCC(=O)NCCc1ccccc1]

To a stirred mixture of 3-[(tert-butoxycarbonyl) amino]propanoic acid (1.87 g, 9.90 mmol, 1.0 eq) and DIEA (2.13 g, 16.5 mmol, 1.5 eq), HATU (4.71 g, 12.4 mmol, 1.2 eq) in DMF (10 mL) was added 2-phenylethylamine (1.0 g, 8.25 mmol, 0.83 eq) in several portions at room temperature and the reaction mixture was stirred at room temperature for 3 h. The resulting mixture was quenched with water (50 mL) and extracted with AcOEt (150 mL). The organic layer was washed with brine (50 mL), dried over anhydrous Na₂SO₄. After filtration, the filtrate was concentrated under reduced pressure and the crude residue was purified by silica gel column chromatography (AcOEt) to afford tert-butyl-N-{2-[(2-phenylethyl)carbamoyl]ethyl}carbamate 39a (1.00 g, 41%) as a white solid. LCMS [column: Kinetex XB-C18,30*3.0 mm, 1.7 μm; mobile phase A: water/0.1% FA, mobile phase B: CAN/0.05% FA, 5%-95% B-2 min (+) ES, m/z]: TR=1.00 min; [M+H]⁺: 293.2.

### tert-Butyl N-{2-[(2-phenylethyl)carbamoyl]ethyl}carbamate (40a)

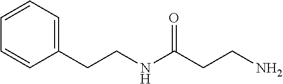 [SMILES: NCCC(=O)NCCc1ccccc1]

A solution of tert-butyl N-{2-[(2-phenylethyl)carbamoyl]ethyl}carbamate 39a (1.0 g, 3.42 mmol, 1.0 eq) in HCl (4M in dioxane, 10 mL) was stirred for 1 h at room temperature. The precipitated solids were collected by filtration and washed with Et₂O (50 mL) to afford 3-amino-N-[(4Z)-3-methylidenehex-4-en-1-yl]propanamide 40a (500 mg, 80%) as a white solid. LCMS [column: Kinetex XB-C18,30*3.0 mm, 1.7 μm. mobile phase A: water/0.1% FA, mobile phase B: CAN/0.05% FA, 5%-95% B-2 min (+), ES, m/z]: TR=0.50 min; [M+H]⁺: 193.1.

### A mixture of 3-((((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl) amino)-N phenethylpropanamide and 3-((((3αR,4S,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl) amino)-N-phenethylpropanamide (41a)

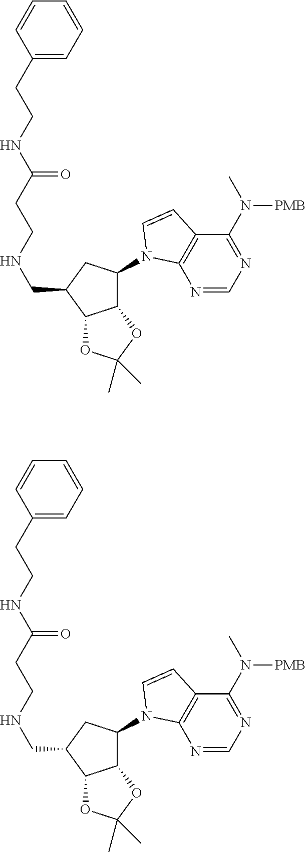

To a stirred solution of (3αR,4S,6R,6αS)-6-(4-{[(4-methoxyphenyl)methyl](methyl) amino}pyrrolo[2,3-d]pyrimidin-7-yl)-2,2-dimethyl-tetrahydro-3aH-cyclopenta[d][1,3]dioxole-4-carbaldehyde 19a (100 mg, 0.23 mmol, 1.0 eq) and 3-amino-N-phenethylpropanamide 40a (52.8 mg, 0.28 mmol, 1.2 eq) in DCE (5.0 mL) was added NaBH (AcO)₃ (121 mg, 0.57 mmol, 2.5 eq) in several portions at room temperature and the resulting mixture was stirred for 12 h. The reaction was quenched by the addition of sodium bicarbonate aqueous solution (0.5 mL). The crude mixture was concentrated and purified by silica gel column chromatography (DCM/MeOH=80/20) to afford the mixture of 3-((((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl) amino)-N-phenethylpropanamide and 3-((((3αR,4S,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl) amino)-N-phenethylpropanamide 41a (130 mg, 56%) as a white solid. LCMS [column: HPH-C18,50*3.0 mm, 2.7 μm. mobile phase A: water/0.05% ammonia water, mobile phase B: ACN, 5%-95%-3 min; ES, m/z]: TR=1.65 min, 1.69 min; 2 diastereomers (1:1) [M+H]⁺: 613.2.

### The mixture of 3-((((1R,2R,3S,4R)-2,3-dihydroxy-4-(4-(methylamino)-7H-pyrrolo[2,3-d]pyrimidin-7-yl) cyclopentyl)methyl) amino)-N-phenethylpropanamide and 3-((((1S,2R,3S,4R)-2,3-dihydroxy-4-(4-(methylamino)-7H-pyrrolo[2,3-d]pyrimidin-7-yl) cyclopentyl)methyl) amino)-N-phenethylpropanamide (434)

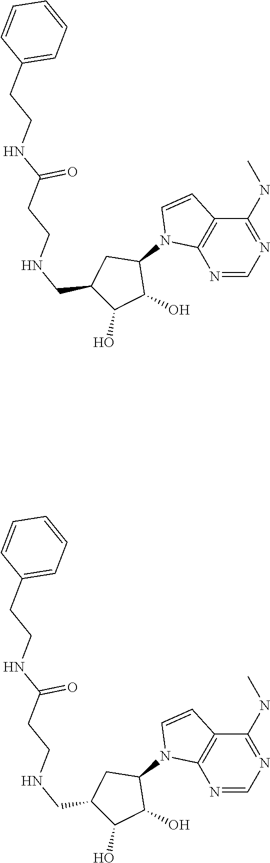 [SMILES: CNc1ncnc2c1ccn2[C@@H]1C[C@H](CNCCC(=O)NCCc2ccccc2)[C@@H](O)[C@H]1O ; CNc1ncnc2c1ccn2[C@@H]1C[C@@H](CNCCC(=O)NCCc2ccccc2)[C@@H](O)[C@H]1O]

To the mixture of 3-((((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl) amino)-N-phenethylpropanamide and 3-((((3αR,4S,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl) amino)-N-phenethylpropanamide 41a (100 mg, 0.16 mmol, 1.0 eq) was added TFA (3.0 mL) and the reaction mixture was stirred for 3 h at room temperature. The resulting mixture was concentrated under vacuum and the crude residue was purified by Prep-HPLC [column, Xbridge Prep C18 OBD column, 5 μm, 19*150 mm; mobile phase, water (0.03% NH₄OH) and CH₃CN (15% up to 38% in 10 min); Detector, UV 220&254 nm]: to afford a mixture of 3-((((1R,2R,3S,4R)-2,3-dihydroxy-4-(4-(methylamino)-7H-pyrrolo[2,3-d]pyrimidin-7-yl) cyclopentyl)methyl) amino)-N-phenethylpropanamide and 3-((((1S,2R,3S,4R)-2,3-dihydroxy-4-(4-(methylamino)-7H-pyrrolo[2,3-d]pyrimidin-7-yl) cyclopentyl)methyl) amino)-N-phenethylpropanamide 434 (15 mg, 20%) as a white solid. ¹H NMR (300 MHZ, DMSO-d₆) δ 8.11 (d, J=2.8 Hz, 1H), 7.34-7.11 (m, 6H), 6.52 (t, J=3.5 Hz, 1H), 4.90-4.84 (m, 1H), 4.40-4.11 (m, 1H), 3.95-3.74 (m, 1H), 3.32-3.18 (m, 2H), 2.94 (s, 3H), 2.77-2.61 (m, 5H), 2.49-2.38 (m, 1H), 2.30-2.24 (m, 1H), 2.23-2.12 (m, 2H), 2.08-1.40 (m, 2H). LCMS [column: HPH-C18,50*3.0 mm, 2.7 μm; mobile phase A: water/0.05% ammonia water, mobile phase B: ACN, 5%-95% B-3 min (+), ES, m/z]: TR=1.36 min; [M+H]⁺: 426.2. Chiral-HPLC [Column: CHIRALPAK IH-3, 50*4.6 mm, 3 μm IH30CC-WH004, Mobile Phase A: n-Hexane (0.1% DEA), Mobile Phase B: Ethanol (0.2% MIPA); Total Flow: 1.00 mL/min; Conc. of Pump B: 30.0% Oven Temperature: 25° C.]: two diastereomers; TR=2.42, 3.44 min. dr 49:51.

### Synthesis of (1R,2S,3R,5R)-3-[4-(methylamino) pyrrolo[2,3-d]pyrimidin-7-yl]-5-{[(morpholin-2-ylmethyl) amino]methyl}cyclopentane-1,2-diol (435)

### (3αR,4S,6R,6αS)-6-(4-((4-Methoxybenzyl)(methyl) amino)-7H-pyrrolo[2,3-d]pyrimidin-7-yl)-2,2-dimethyltetrahydro-4H-cyclopenta[d][1,3]dioxole-4-carbaldehyde (19a)

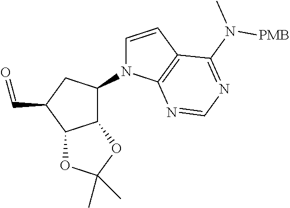

A solution of [(3αR,4R,6R,6αS)-6-(4-{[(4-methoxyphenyl)methyl](methyl) amino}pyrrolo[2,3-d]pyrimidin-7-yl)-2,2-dimethyl-tetrahydro-3aH-cyclopenta[d][1,3]dioxol-4-yl]methanol (Intermediate A, 600 mg, 1.37 mmol, 1.0 eq) and DMP (1.16 g, 2.73 mmol, 2.0 eq) in DCM (5.0 mL) and the reaction mixture was stirred for 4 h at room temperature. The crude mixture was purified by silica gel column chromatography (AcOEt) to afford (3αR,4S,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxole-4-carbaldehyde 19a (430 mg, 36%) as a yellow oil. ¹H NMR (300 MHZ, DMSO-d₆) δ 9.73 (s, 1H), 8.16 (s, 1H), 8.14-7.95 (m, 1H), 7.32 (d, J=3.7 Hz, 1H), 7.25-7.13 (m, 2H), 6.94-6.83 (m, 2H), 6.57 (d, J=3.7 Hz, 1H), 5.15-5.02 (m, 2H), 4.94 (d, J=1.6 Hz, 2H), 3.72 (s, 3H), 3.27 (s, 3H), 3.16-3.02 (m, 1H), 2.48-2.31 (m, 1H), 1.50 (s, 3H), 1.26 (s, 3H).

### tert-Butyl-2-(((((3αR,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl) amino) methyl) morpholine-4-carboxylate (20a)

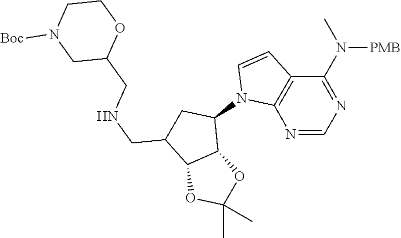

To a stirred solution of (3αR,4S,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxole-4-carbaldehyde 19 (100 mg, 0.23 mmol, 1.0 eq) and racemic tert-butyl-2-(aminomethyl) morpholine-4-carboxylate (59.5 mg, 0.28 mmol, 1.2 eq) in DCE (3.0 mL) was added NaBH (AcO)₃ (1.21 g, 0.57 mmol, 2.5 eq) in several portions at room temperature and the reaction mixture was stirred for 2 h at room temperature. The reaction was quenched with water (0.3 mL) and the resulting mixture was purified by silica gel column chromatography (80:20 DCM/MeOH) to afford tert-butyl-2-(((((3αR,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl) amino) methyl) morpholine-4-carboxylate 20a (150 mg, 82%) as a yellow oil. LCMS [column: Kinetex XB-C18,3.0*30 mm, 1.7 μm, Mobile Phase A: water/0.1% FA, Mobile Phase B: ACN/0.05% FA, 5%-95% B-2 min (+), 1.50 mL/min, ES, m/z]: TR=0.86 min; [M+H]⁺: 637. Epimerisation occured at this step but we saw no evedince by LCMS or chiral HPLC.

### The mixture of (1R,2S,3R,5R)-3-(4-(methylamino)-7H-pyrrolo[2,3-d]pyrimidin-7-yl)-5-(((morpholin-2-ylmethyl) amino) methyl) cyclopentane-1,2-diol and (1R,2S,3R,5S)-3-(4-(methylamino)-7H-pyrrolo[2,3-d]pyrimidin-7-yl)-5-(((morpholin-2-ylmethyl) amino) methyl) cyclopentane-1,2-diol (435)

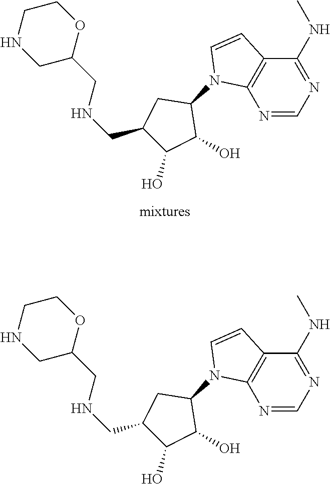 [SMILES: CNc1ncnc2c1ccn2[C@@H]1C[C@H](CNCC2CNCCO2)[C@@H](O)[C@H]1O ; CNc1ncnc2c1ccn2[C@@H]1C[C@@H](CNCC2CNCCO2)[C@@H](O)[C@H]1O]

To tert-butyl-2-(((((3αR,4R,6R,6αS)-2,2-dimethyl-6-(4-(methylamino)-7H-pyrrolo[2,3-d]pyrimidin-7-yl) tetrahydro-4H-cyclopenta[d][1,3]dioxol-4-yl)methyl) amino) methyl) morpholine-4-carboxylate 20 (100 mg, 0.21 mmol, 1.0 eq) was added TFA (3.0 mL) and the reaction mixture was stirred for 2 h at room temperature. The resulting mixture was concentrated under vacuum and the crude residue was purified by Prep-HPLC [column: Xbridge Prep C18 OBD column, 5 μm, 19*150 mm; mobile phase, Water (0.03% NH₄OH) and CH₃CN (10% up to 35% in 8 min); Detector, UV 220&254 nm]: to afford a mixture of (1R,2S,3R,5R)-3-[4-(methylamino) pyrrolo[2,3-d]pyrimidin-7-yl]-5-{[(morpholin-2-ylmethyl) amino]methyl}cyclopentane-1,2-diol and (1R,2S,3R,5S)-3-(4-(methylamino)-7H-pyrrolo[2,3-d]pyrimidin-7-yl)-5-(((morpholin-2-ylmethyl) amino) methyl) cyclopentane-1,2-diol 435 (22 mg, 28%) as a white solid. ¹H NMR (300 MHZ, Methanol-d₄) δ 8.15 (s, 1H), 7.26-7.18 (m, 1H), 6.58 (br s, 1H), 5.06-4.94 (m, 0.4H), 5.53-4.45 (m, 0.6H), 4.32-4.08 (m, 1H), 4.07-3.81 (m, 2H), 3.73-3.54 (m, 2H), 3.07 (br s, 3H), 3.06-2.67 (m, 7H), 2.63-2.48 (m, 1H), 2.47-2.20 (m, 1H), 2.17-2.02 (m, 1H), 1.79-1.51 (m, 1H). LCMS [column: L-column C18, 50*3.0 mm, 3 μm. mobile phase A: water/0.05% ammonia water, mobile phase B: ACN, 5%-40%-95% B-5.0 min (+); 1.50 mL/min, ES, m/z]: TR=1.31 min, TR=1.35 min; [M+H]⁺: 377.2. Chiral-HPLC [Column: CHIRALPAK IG-3, 100*4.6 mm, 3 μm IG30CS-UL011; Mobile Phase A: n-Hexane/DCM=3/1; Mobile Phase B: Ethanol (0.2% MIPA), Total Flow: 1.00 mL/min; Conc. of Pump B: 50.0%; Oven Temperature: 25° C., (12 min run)]: 4 diastereomers; TR=4.32, 5.22, 7.18, 8.91 min; dr=21:30:20:29.

### Synthesis of (1R,2,3R,5R)-3-[4-(methylamino) pyrrolo[2,3-d]pyrimidin-7-yl]-5-({[(3S)-piperidin-3-ylmethyl]amino}methyl) cyclopentane-1,2-diol (436) (3αR,4S,6R,6αS)-6-{4-Chloropyrrolo[2,3-d]pyrimidin-7-yl}-2,2-dimethyl-tetrahydro-3αH-cyclopenta[d][1,3]dioxole-4-carbaldehyde (77a)

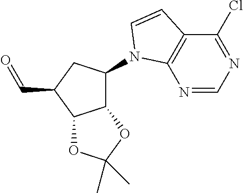 [SMILES: CC1(C)O[C@@H]2[C@H](O1)[C@@H](C=O)C[C@H]2n1ccc2c(Cl)ncnc21]

To [(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methanol 5 (0.50 g, 1.50 mmol, 1.0 eq) in DCM (5.0 mL) was added DMP (0.85 g, 2.00 mmol, 1.3 eq) and the reaction mixture was stirred for 1 h at room temperature. The crude residue was purified by silica gel column chromatography (80:20 Petroleum ether/AcOEt) to afford (3αR,4S,6R,6αS)-6-{4-chloropyrrolo[2,3-d]pyrimidin-7-yl}-2,2-dimethyl-tetrahydro-3αH-cyclopenta[d][1,3]dioxole-4-carbaldehyde 77a (0.25 g, 50%) as an off-white solid. LCMS [conditions XBridge C18, 50*3.0 mm, 3.5 μm, Mobile Phase A: Water/5 mM NH₄HCO₃, Mobile Phase B: Methanol, 10%-95% B-2 min (+), 1.50 L/min, ES, m/z]: TR=1.58 min; [M+H]⁺: 322.

### tert-Butyl-(3R)-3-[{{[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}amino) methyl]piperidine-1-carboxylate (78a)

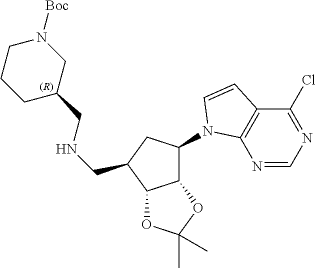

To a solution of tert-butyl-(3R)-3-(aminomethyl) piperidine-1-carboxylate (0.18 g, 0.90 mmol, 1.2 eq) in DCM (1.0 ml) was added NaBH (OAc)₃ (0.30 g, 1.40 mmol, 2.0 eq) and HOAc (0.43 g, 7.10 mmol, 10.0 eq) and the reaction mixture was stirred for 10 min at room temperature. To this mixture was added (3αR,4S,6R,6αS)-6-{4-chloropyrrolo[2,3-d]pyrimidin-7-yl}-2,2-dimethyl-tetrahydro-3αH-cyclopenta[d][1,3]dioxole-4-carbaldehyde 77 (0.23 g, 0.70 mmol, 1.0 eq) in DCM (1.0 mL), dropwise over 2 min, at 0° C., and the reaction mixture was stirred for 30 min at room temperature and the resulting mixture was concentrated under vacuum. The crude residue was dissolved in water (2.0 mL) and basified to pH>7 with TEA and purified by Prep-HPLC [Column: Xtimate C18 50×250 mm, 10 μm; Mobile Phase A: Water (0.1% NH₃.H₂O), Mobile Phase B: ACN; Flow rate: 90 mL/min; Gradient: 35% B to 65% B in 12 min, 65% B; Wave Length: 220 nm]: to afford tert-butyl-(3R)-3-[{{[(3αR,4R,6R,6αS)-6-{4-chloropyrrolo[2,3-d]pyrimidin-7-yl}-2,2-dimethyl-tetrahydro-3H-cyclopenta[d][1,3]dioxol-4-yl]methyl}amino) methyl]piperidine-1-carboxylate 78a (0.16 g, 43%) as an off-white solid. LCMS (conditions XBridge C18, 50*3.0 mm, 3.5 μm, Mobile Phase A: Water/5 mM NH₄HCO₃, Mobile Phase B: Methanol, 10%-95% B-2 min (+), 1.50 L/min, ES, m/z): TR=3.81 min; [M+H]⁺: 520.

### tert-Butyl(3R)-3-[{{[(3αR,4R,6R,6αS)-6-(4-{[(4-methoxyphenyl)methyl](methyl) amino}pyrrolo[2,3-d]pyrimidin-7-yl)-2,2-dimethyl-tetrahydro-3H-cyclopenta[d][1,3]dioxol-4-yl]methyl}amino) methyl]piperidine-1-carboxylate (79a)

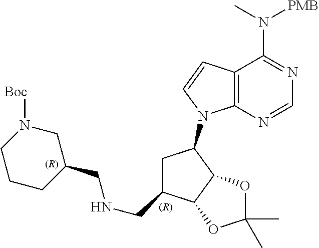

To tert-butyl-(3R)-3-[{{[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}amino) methyl]piperidine-1-carboxylate 78a (0.16 g, 0.30 mmol, 1.0 eq), in ethanol (2.0 mL), was added [(4-methoxyphenyl)methyl](methyl) amine (0.28 g, 1.80 mmol, 6.0 eq) at room temperature and the resulting mixture was stirred at 80° C. for 16 h. The crude residue was purified by Prep-HPLC to afford tert-butyl (3R)-3-[{{[(3αR,4R,6R,6αS)-6-(4-{[(4-methoxyphenyl)methyl](methyl) amino}pyrrolo[2,3-d]pyrimidin-7-yl)-2,2-dimethyl-tetrahydro-3aH-cyclopenta[d][1,3]dioxol-4-yl]methyl}amino) methyl]piperidine-1-carboxylate 79a (0.90 g, 46%) as an off-white solid. LCMS [conditions XBridge C18, 50*3.0 mm, 3.5 μm, Mobile Phase A: Water/5 mM NH₄HCO₃, Mobile Phase B: Methanol, 10%-95% B-5 min (+), 1.50 L/min, ES, m/z]: TR=4.00 min; [M+H]⁺: 635.

### (1R,2S,3R,5R)-3-[4-(Methylamino) pyrrolo[2,3-d]pyrimidin-7-yl]-5-({[(3S)-piperidin-3-ylmethyl]amino}methyl) cyclopentane-1,2-diol (436)

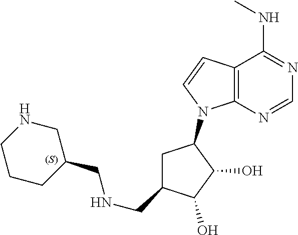

To tert-butyl-(3R)-3-[{{[(3αR,4R,6R,6αS)-6-(4-{[(4-methoxyphenyl)methyl](methyl) amino}pyrrolo[2,3-d]pyrimidin-7-yl)-2,2-dimethyl-tetrahydro-3αH-cyclopenta[d][1,3]dioxol-4-yl]methyl}amino) methyl]piperidine-1-carboxylate 79a (0.04 g, 0.10 mmol, 1.0 eq) was added TFA (1.0 mL) at room temperature and the reaction mixture was stirred for 30 min at 50° C. The resulting mixture was concentrated under vacuum and the crude residue was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 24% B to 60% B in 7 min, 60% B; Wave Length: 220 nm; TR (min): 6.9]: to afford (1R,2S,3R,5R)-3-[4-(methylamino) pyrrolo[2,3-d]pyrimidin-7-yl]-5-({[(3S)-piperidin-3-ylmethyl]amino}methyl) cyclopentane-1,2-diol 436 (10 mg, 42%) as an off-white solid. ¹H NMR (300 MHZ, DMSO-d₆) δ 8.11 (s, 1H), 8.09 (s, 1H), 7.21 (d, J=3.5 Hz, 1H), 6.52 (d, J=3.5 Hz, 1H), 4.87-4.73 (m, 1H), 4.18-4.11 (m, 1H), 3.80-3.71 (m, 1H), 3.05-2.77 (m, 5H), 2.75-2.59 (m, 1H), 2.58-2.48 (m, 1H), 2.44-2.26 (m, 3H), 2.25-1.91 (m, 3H), 1.87-1.64 (m, 1H), 1.61-1.43 (m, 3H), 1.42-1.24 (m, 1H), 1.11-0.85 (m, 1H). LCMS [conditions Atlantis T3, 100*4.6 mm, 3 μm, Mobile Phase A: Water/0.02% TFA, Mobile Phase B: Acetonitrile, 0%-30%-95% B-7 min, 1.50 L/min, ES, m/z]: TR=3.35 min; [M+H]⁺: 375 Chiral-SFC [Column: Lux-4 100×4.6 mm 3.0 μm, Solvent B: MeOH (20 mM NH₃), Oven Temperature: 35° C., Total Flow: 3.00 mL/min]: TR=2.54 min, single peak.

### Synthesis of (1S,2R,3R,5R)-3-{[(3-aminopropyl)(1,3-thiazol-5-yl) amino]methyl}-5-[4-(methylamino) pyrrolo[2,3-d]pyrimidin-7-yl]cyclopentane-1,2-diol (437)

### N-{[(3αR,4R,6R,6αS)-6-{4-Chloropyrrolo[2,3-d]pyrimidin-7-yl}-2,2-dimethyl-tetrahydro-3αH-cyclopenta[d][1,3]dioxol-4-yl]methyl}-1.3-thiazol-5-amine (89a)

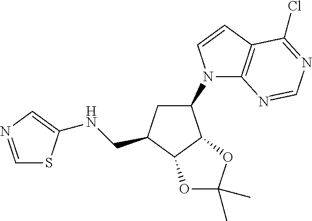 [SMILES: CC1(C)O[C@@H]2[C@@H](CNc3cncs3)C[C@@H](n3ccc4c(Cl)ncnc43)[C@@H]2O1]

To a solution of 1,3-thiazol-5-amine hydrochloride (95 mg, 0.70 mmol, 1.5 eq) in DCM (1.0 mL), at 0° C., was added AcOH (0.28 g, 4.70 mmol, 10 eq) and NaBH (OAc)₃ (0.20 g, 0.90 mmol, 2.0 eq) and the resulting mixture was stirred for 30 min at 0° C. Then (3αR,4S,6R,6αS)-6-{4-chloropyrrolo[2,3-d]pyrimidin-7-yl}-2,2-dimethyl-tetrahydro-3αH-cyclopenta[d][1,3]dioxole-4-carbaldehyde 77 (0.15 g, 0.50 mmol, 1.0 eq) in DCM (1.0 mL) were added dropwise, over 3 min at 0° C. and the reaction mixture was stirred for 30 min at 0° C. The resulting mixture was concentrated under vacuum and the crude residue was purified by silica gel column chromatography (96:4 DCM: MeOH) to afford NH {[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}-1,3-thiazol-5-amine 89a (0.15 g, 79%) as a light brown solid. LCMS [conditions XBridge C18, 50*3.0 mm, 3.5 μm, Mobile Phase A: Water/5 mM NH₄HCO₃, Mobile Phase B: Methanol, 10%-95% B-2 min (+), 1.50 L/min, ES, m/z]: TR=1.21 min; [M+H]⁺: 406

### N-[3-({[(3αR,4R,6R,6αS)-2,2-Dimethyl-6-[4-(methylamino) pyrrolo[2,3-d]pyrimidin-7-yl]-tetrahydro-3αH-cyclopenta[d][1,3]dioxol-4-yl]methyl}(1,3-thiazol-5-yl) amino)propyl]carbamate (90a)

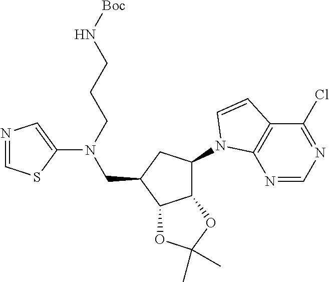 [SMILES: CC(C)(C)OC(=O)NCCCN(C[C@H]1C[C@@H](n2ccc3c(Cl)ncnc32)[C@@H]2OC(C)(C)O[C@H]12)c1cncs1]

To a solution of N-{[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}-1,3-thiazol-5-amine 89a (0.10 g, 0.25 mmol, 1.0 eq) in DCM (1.0 mL), at 0° C., was added HOAc (0.15 g, 2.50 mmol, 10 eq) and NaBH (OAc)₃ (0.10 g, 0.50 mmol, 2.0 eq) and the resulting mixture was stirred for 30 min at 0° C. To the above mixture was added a solution of tert-butyl-(3S)-3-formylpiperidine-1-carboxylate (0.12 g, 0.60 mmol, 1.5 eq) in DCM (1.0 mL), dropwise over 3 min, at 0° C. and the reaction mixture was stirred for an additional 30 min at 0° C. The resulting mixture was concentrated under vacuum and the crude mixture was purified by silica gel column chromatography (95:5 DCM: MeOH) to afford tert-butyl-N-[3-({[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}(1,3-thiazol-5-yl) amino)propyl]carbamate 90a (0.10 g, 72%) as a light brown oil. LCMS [conditions XBridge C18, 50*3.0 mm, 3.5 μm, Mobile Phase A: Water/5 mM NH₄HCO₃, Mobile Phase B: Methanol, 10%-95% B-2 min (+), 1.50 L/min, ES, m/z]: T_{R}=1.50 min; [M+H]⁺: 563.

### tert-Butyl-N-[3-({[(3αR,4R,6R,6αS)-2,2-dimethyl-6-[4-(methylamino) pyrrolo[2,3-d]pyrimidin-7-yl]-tetrahydro-3αH-cyclopenta[d][1,3]dioxol-4-yl]methyl}(1,3-thiazol-5-yl) amino)propyl]carbamate (91a)

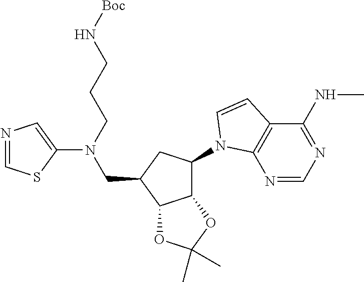 [SMILES: CNc1ncnc2c1ccn2[C@@H]1C[C@H](CN(CCCNC(=O)OC(C)(C)C)c2cncs2)[C@H]2OC(C)(C)O[C@H]21]

To tert-butyl-N-[3-({[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}(1,3-thiazol-5-yl) amino)propyl]carbamate 90a (0.10 g, 0.20 mmol, 1.0 eq) was added methylamine (30% in ethanol, 2.0 mL) and the reaction mixture was stirred for 2 h at 80° C. The resulting mixture was concentrated under vacuum and the residue was purified by Prep-HPLC [Column: Xtimate C18 50×250 mm, 10 μm; Mobile Phase A: Water (0.1% NH₃.H₂O), Mobile Phase B: ACN; Flow rate: 90 mL/min; Gradient: 35% B to 60% B in 12 min, 60% B; Wave Length: 220 nm]: to afford tert-butyl-N-[3-({[(3αR,4R,6R,6αS)-2,2-dimethyl-6-[4-(methylamino) pyrrolo[2,3-d]pyrimidin-7-yl]-tetrahydro-3αH-cyclopenta[d][1,3]dioxol-4-yl]methyl}(1,3-thiazol-5-yl) amino)propyl]carbamate 91a (80 mg, 81%) as an off-white solid. LCMS [conditions Cortecs C_{18+, 50*3.0} mm, 2.7 μm, Mobile Phase A: Water/0.05% TFA, Mobile Phase B: Acetonitrile, 5%-95% B-5 min, 1.50 L/min, ES, m/z]: T_{R}=2.64 min; [M+H]⁺: 558.

### (1S,2R,3R,5R)-3-{[(3-Aminopropyl)(1,3-thiazol-5-yl) amino]methyl}-5-[4-(methylamino) pyrrolo[2,3-d]pyrimidin-7-yl]cyclopentane-1,2-diol (437)

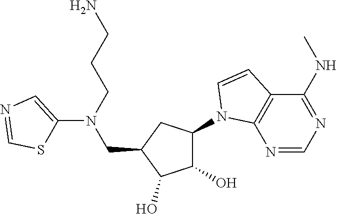 [SMILES: CNc1ncnc2c1ccn2[C@@H]1C[C@H](CN(CCCN)c2cncs2)[C@@H](O)[C@H]1O]

To tert-butyl-N-[3-({[(3αR,4R,6R,6αS)-2,2-dimethyl-6-[4-(methylamino) pyrrolo[2,3-d]pyrimidin-7-yl]-tetrahydro-3αH-cyclopenta[d][1,3]dioxol-4-yl]methyl}(1,3-thiazol-5-yl) amino)propyl]carbamate 91a (80 mg, 0.14 mmol, 1.0 eq) was added HCl (4M in 1,4-dioxane, 2.0 mL) and the reaction mixture was stirred for 2 h at room temperature. The resulting mixture was concentrated under vacuum, the residue was dissolved in methanol (1.0 mL) and neutralized to pH>7 with NH₃.H₂O. The crude residue was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: Acetonitrile; Flow rate: 30 mL/min; Gradient: 24% B to 45% B in 7 min, 45% B; Wave Length: 220 nm]: to afford (1S,2R,3R,5R)-3-{[(3-aminopropyl)(1,3-thiazol-5-yl) amino]methyl}-5-[4-(methylamino) pyrrolo[2,3-d]pyrimidin-7-yl]cyclopentane-1,2-diol 437 (31.1 mg, 52%) as an off-white solid. ¹H NMR (300 MHZ, DMSO-d₆+D₂O) 8 8.18 (s, 1H), 8.09 (s, 1H), 7.24 (d, J=3.6 Hz, 1H), 6.91 (s, 1H), 6.52 (d, J=3.5 Hz, 1H), 4.79 (q, J=8.8 Hz, 1H), 4.32-4.22 (m, 1H), 3.77 (t, J=4.9 Hz, 1H), 3.53-3.34 (m, 1H), 3.31-3.12 (m, 3H), 2.93 (s, 3H), 2.61-2.52 (m, 2H), 2.29 (s, 1H), 2.23-2.07 (m, 1H), 1.71-1.57 (m, 2H), 1.56-1.40 (m, 1H). LCMS [conditions ZORBAX SB-Aq, 50*4.6 mm, 1.8 μm, Mobile Phase A: Water/0.02% TFA, Mobile Phase B: Acetonitrile, 5%-95% B-3 min, 1.50 L/min, ES, m/z]: T_{R}=0.93 min; [M+H]⁺: 418. Chiral HPLC [CHIRALPAK IC-3, 50*4.6 mm, 3 μm, Mobile Phase A: Water/0.05% MSA, Mobile Phase B: CAN, Total Flow: 1.00 mL/min]: T_{R}=0.87 (major isomer), T_{R}=2.08 (minor isomer), dr>99:1.

### Synthesis of (1R,2S,3R,5R)-3-[4-(Methylamino) pyrrolo[2,3-d]pyrimidin-7-yl]-5-({[(3S)-piperidin-3-ylmethyl](1,3-thiazol-5-yl) amino}methyl) cyclopentane-1,2-diol (438)

### N-{[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}-1,3-thiazol-5-amine (80a)

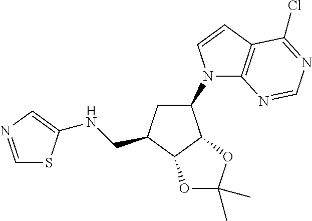 [SMILES: CC1(C)O[C@@H]2[C@@H](CNc3cncs3)C[C@@H](n3ccc4c(Cl)ncnc43)[C@@H]2O1]

To 1,3-thiazol-5-amine hydrochloride (0.09 g, 0.70 mmol, 1.5 eq), in DCM (1.0 mL) and AcOH (0.28 g, 4.70 mmol, 10.0 eq), was added NaBH (OAc)₃ (0.20 g, 0.90 mmol, 2.0 eq) and the resulting mixture was stirred for 30 min at 0° C. To this mixture was added (3αR,45,6R,6αS)-6-{4-chloropyrrolo[2,3-d]pyrimidin-7-yl}-2,2-dimethyl-tetrahydro-3αH-cyclopenta[d][1,3]dioxole-4-carbaldehyde 5 (0.15 g, 0.50 mmol, 1.0 eq) in DCM (1.0 mL), dropwise, over 3 min, at 0° C., and the reaction mixture was stirred for 30 min at 0° C. The crude residue was purified by silica gel column chromatography (94:6 DCM: MeOH) to afford N-{[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}-1,3-thiazol-5-amine 80a (0.15 g, 79%) as a light brown solid. LCMS [conditions Cortecs C18+, 50*3.0 mm, 2.7 μm, Mobile Phase A: Water/5 mM NH₄HCO₃, Mobile Phase B: Methanol, 10%-95% B-2 min (+), 1.50 L/min, ES, m/z, ES, m/z]: T_{R}=1.33 min; [M+H]⁺: 406. tert-Butyl-(3S)-3-[{{[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}(1,3-thiazol-5-yl) amino) methyl]piperidine-1-carboxylate (81a)

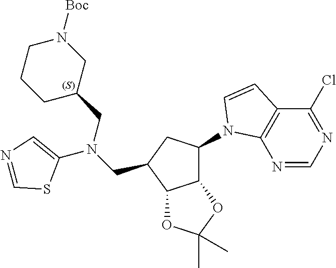

To N-{[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}-1,3-thiazol-5-amine 80a (0.15 g, 0.40 mmol, 1.0 eq) in DCM (1.0 mL) and AcOH (0.22 g, 3.70 mmol, 10.0 eq) was added NaBH (OAc)₃ (0.16 g, 0.70 mmol, 2.0 eq) and the resulting mixture was stirred for 30 min at 0° C. To the above mixture was added tert-butyl-(3S)-3-formylpiperidine-1-carboxylate (0.12 g, 0.50 mmol, 1.5 eq) in DCM (1.0 mL), dropwise over 3 min, at 0° C. and the reaction mixure was stirred for 30 mins at 0° C. The resulting mixture was purified by silica gel column chromatography (93:7 DCM: MeOH) to afford tert-butyl-(3S)-3-[{{[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}(1,3-thiazol-5-yl) amino) methyl]piperidine-1-carboxylate 81a (0.12 g, 54%) as a light brown oil. LCMS [conditions XBridge C18, 50*3.0 mm, 3.5 μm, Mobile Phase A: Water/5 mM NH₄HCO₃, Mobile Phase B: Methanol, 10%-95% B-2 min (+), 1.50 L/min, ES, m/z): T_{R}=1.51 min; [M+H]⁺: 603

### (3S)-3-[{{[(3αR,4R,6R,6αS)-2,2-Dimethyl-6-[4-(methylamino) pyrrolo[2,3-d]pyrimidin-7-yl]-tetrahydro-3αH-cyclopenta[d][1,3]dioxol-4-yl]methyl}(1,3-thiazol-5-yl) amino) methyl]piperidine-1-carboxylate (82a)

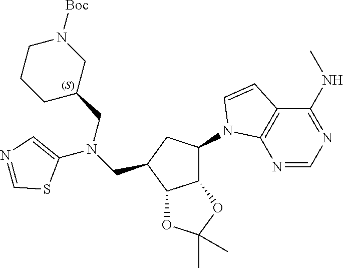

To tert-butyl-(3S)-3-[{{[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}(1,3-thiazol-5-yl) amino) methyl]piperidine-1-carboxylate 81a (0.12 g, 0.20 mmol, 1.0 eq) in methylamine (30% in ethanol, 2.0 mL). The resulting mixture was stirred for 2 h at 80° C. The residue was purified by Prep-HPLC to afford tert-butyl (3S)-3-[{{[(3αR,4R,6R,6αS)-2,2-dimethyl-6-[4-(methylamino) pyrrolo[2,3-d]pyrimidin-7-yl]-tetrahydro-3αH-cyclopenta[d][1,3]dioxol-4-yl]methyl}(1,3-thiazol-5-yl) amino) methyl]piperidine-1-carboxylate 82a (0.10 g, 84%) as an off-white solid. LCMS [conditions XBridge C18, 50*3.0 mm, 3.5 μm, Mobile Phase A: Water/5 mM NH₄HCO₃, Mobile Phase B: Methanol, 10%-95% B-5 min (+), 1.50 L/min, ES, m/z]: T_{R}=3.57 min; [M+H]⁺: 598

### (1R,2S,3R,5R)-3-[4-(Methylamino) pyrrolo[2,3-d]pyrimidin-7-yl]-5-({[(3S)-piperidin-3-ylmethyl](1,3-thiazol-5-yl) amino}methyl) cyclopentane-1,2-diol (438)

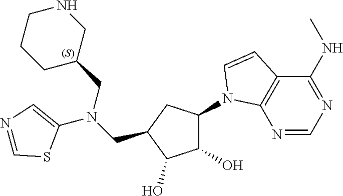

To tert-butyl-(3S)-3-[{{[(3αR,4R,6R,6αS)-2,2-dimethyl-6-[4-(methylamino) pyrrolo[2,3-d]pyrimidin-7-yl]-tetrahydro-3αH-cyclopenta[d][1,3]dioxol-4-yl]methyl}(1,3-thiazol-5-yl) amino) methyl]piperidine-1-carboxylate 82a (0.10 g, 0.20 mmol, 1.0 eq) was added HCl (4 M in 1,4-dioxane, 2.0 mL) and the reaction mixture was stirred for 1 h at room temperature. The resulting mixture was concentrated under vacuum, the crude residue was dissolved in methanol and the solution was neutralized to pH>7 with NH₄.H₂O. The crude residue was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 20% B to 50% B in 8 min, 49% B; Wave Length: 220 nm]: to afford (1R,2S,3R,5R)-3-[4-(methylamino) pyrrolo[2,3-d]pyrimidin-7-yl]-5-({[(3S)-piperidin-3-ylmethyl](1,3-thiazol-5-yl) amino}methyl) cyclopentane-1,2-diol 438 (32.7 mg, 43%) as an off-white solid. ¹H NMR (300 MHz, DMSO-d₆+D₂O) 88.19 (s, 1H), 8.10 (s, 1H), 7.24 (d, J=3.6 Hz, 1H), 6.91 (s, 1H), 6.52 (d, J=3.5 Hz, 1H), 4.84-4.69 (m, 1H), 4.34-4.21 (m, 1H), 3.82-3.73 (m, 1H), 3.49-3.39 (m, 1H), 3.31-3.17 (m, 1H), 3.11-3.03 (m, 2H), 2.94 (s, 3H), 2.90-2.70 (m, 2H), 2.46-2.25 (m, 2H), 2.24-2.09 (m, 2H), 1.93-1.76 (m, 1H), 1.75-1.61 (m, 1H), 1.60-1.46 (m, 2H), 1.42-1.18 (m, 1H), 1.17-0.96 (m, 1H). LCMS [conditions Cortecs C_{18+, 50*3.0} mm, 2.7 μm, Mobile Phase A: Water/0.02% TFA, Mobile Phase B: Acetonitrile, 5%-95% B-3 min, 1.50 L/min, ES, m/z]: T_{R}=0.55 min; [M+H]⁺: 458. Chiral HPLC [YMC Cellulose SB, 100*4.6 mm, 3 μm, Mobile Phase A: Water/0.05% MSA, Mobile Phase B: CAN, Total Flow: 1.00 mL/min]: T_{R}=1.38 min (major isomer), 1.68 min (minor isomer); dr>99:1.

### Synthesis of (1R,2S,3R,5R)-3-(4-(Methylamino)-7H-pyrrolo[2,3-d]pyrimidin-7-yl)-5-(((3-(phenethylamino)propyl)(thiazol-5-yl) amino) methyl) cyclopentane-1,2-diol (439)

### tert-Butyl-(3-((((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl)(thiazol-5-yl) amino)propyl)(phenethyl)carbamate (69a)

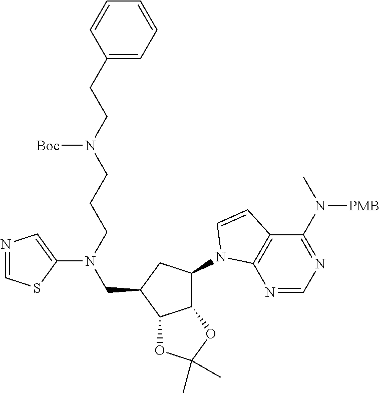

To tert-butyl-N-[3-({[(3αR,4R,6R,6αS)-6-(4-{[(4-methoxyphenyl)methyl](methyl) amino}pyrrolo[2,3-d]pyrimidin-7-yl)-2,2-dimethyl-tetrahydro-3αH-cyclopenta[d][1,3]dioxol-4-yl]methyl}amino)propyl]-N-(2-phenylethyl)carbamate 67a (0.10 g, 0.14 mmol, 1.0 eq) and toluene (5.0 mL) was added 5-bromo-1,3-thiazole (117 mg, 0.72 mmol, 5.0 eq), Pd₂ (dba)₃ (13 mg, 0.02 mmol, 0.1 eq), BINAP (13 mg, 0.02 mmol, 0.15 eq) and t-BuONa (69 mg, 0.72 mmol, 5.0 eq) and the reaction mixture was stirred at 100° C., for 16 h, under N₂. The resulting mixture was concentrated under reduced pressure and the crude residue was purified by reverse flash chromatography [column, C₁₈ silica gel; mobile phase, MeOH in Water/0.05% ammonia water, 50% to 90% gradient in 15 min; detector, UV 220 nm]: to afford tert-butyl-N-[3-({[(3ªR,4R,6R,6αS)-6-(4-{[(4-methoxyphenyl)methyl](methyl) amino}pyrrolo[2,3-d]pyrimidin-7-yl)-2,2-dimethyl-tetrahydro-3αH-cyclopenta[d][1,3]dioxol-4-yl]methyl}(1,3-thiazol-5-yl) amino)propyl]-N-(2-phenylethyl)carbamate 69a (93 mg, 63%) as a brown solid. LCMS [conditions: Kinetex XB-C18, 50*3.0 mm, 2.6 μm, Mobile Phase A: Water/5 mM NH₄HCO₃, Mobile Phase B: Methanol, 10%-95% B (2 min)-4 min, 1.50 mL/min, ES, m/z]: T_{R}=2.49 min; [M+H]⁺: 782.

### (1R,2S,3R,5R)-3-(4-(Methylamino)-7H-pyrrolo[2,3-d]pyrimidin-7-yl)-5-(((3-(phenethylamino)propyl)(thiazol-5-yl) amino) methyl) cyclopentane-1,2-diol (439)

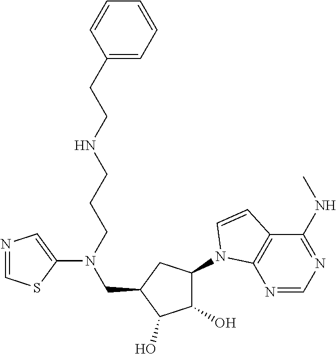 [SMILES: CNc1ncnc2c1ccn2[C@@H]1C[C@H](CN(CCCNCCc2ccccc2)c2cncs2)[C@@H](O)[C@H]1O]

To tert-butyl-N-[3-({[(3αR,4R,6R,6αS)-6-(4-{[(4-methoxyphenyl)methyl](methyl) amino}pyrrolo[2,3-d]pyrimidin-7-yl)-2,2-dimethyl-tetrahydro-3αH-cyclopenta[d][1,3]dioxol-4-yl]methyl}(1,3-thiazol-5-yl) amino)propyl]-N(2-phenylethyl)carbamate 69a (91 mg, 0.12 mmol, 1.0 eq) at 0° C. was added trifluoroacetic acid (2.0 mL) and the reaction mixture was stirred at room temperature for 16 h. The resulting mixture was concentrated under vacuum and the residue was diluted with DMF (2.0 mL) and basified to pH 9-10 with NH₃.H₂O. The crude product was purified by Prep-HPLC [Column: YMC-Actus Triart C₁₈ ExRS, 30*150 mm, 5 μm; Mobile Phase A: Water (10 mmol/L NH₄HCO₃+0.1% NH₃·H₂O), Mobile Phase B: ACN; Flow rate: 30 ml/min; Gradient: 20% B to 49% B in 7 min, 49% B; Wave Length: 220 nm]: to afford (1R,2S,3R,5R)-3-[4-(methylamino) pyrrolo[2,3-d]pyrimidin-7-yl]-5-[({3-[(2-phenylethyl) amino]propyl}(1,3-thiazol-5-yl) amino) methyl]cyclopentane-1,2-diol 439 (16.2 mg, 27%) as an off white solid. ¹H NMR (300 MHZ, Chloroform-d) 88.31 (s, 1H), 8.17 (d, J=0.8 Hz, 1H), 7.41-7.29 (m, 2H), 7.26-7.17 (m, 3H), 7.02 (d, J=0.9 Hz, 1H), 6.93 (d, J=3.6 Hz, 1H), 6.37 (d, J=3.6 Hz, 1H), 5.26-5.06 (m, 1H), 4.78-4.58 (m, 1H), 4.33-4.20 (m, 1H), 4.08-3.98 (m, 1H), 3.47-3.08 (m, 6H), 2.94-2.87 (m, 2H), 2.86-2.78 (m, 2H), 2.77-2.62 (m, 2H), 2.60-2.45 (m, 2H), 1.91-1.65 (m, 3H). LCMS [conditions: ZORBAX SB-Aq, 50*4.6 mm, 1.8 μm, Mobile Phase A: Water/0.05% TFA, Mobile Phase B: Acetonitrile, 5%-50%-95% B-5 min, 1.50 mL/min, ES, m/z]: T_{R}=1.80 min; [M+H]⁺: 522. Chiral-SFC [Column: Lux-4 100×4.6 mm 3.0 μm, Solvent B: MeOH (20 mM NH₃), Oven Temperature: 35° C., Total Flow: 3.00 mL/min]: T_{R}=1.44 min (major isomer), 2.08 min (minor isomer); dr >99:1.

### Synthesis of (1R,2S,3R,5R)-3-(4-(methylamino)-7H-pyrrolo[2,3-d]pyrimidin-7-yl)-5-(((3-(phenethylamino)propyl)(phenyl)amino) methyl) cyclopentane-1,2-diol (440)

### tert-Butyl-(3-((((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl) amino)propyl)(phenethyl)carbamate (67a)

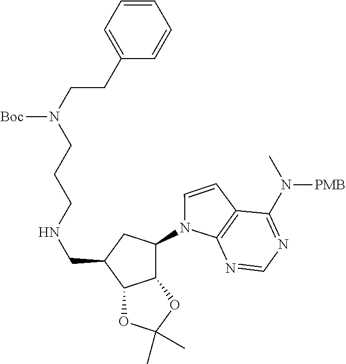

To 7-[(3αS,4R,6R,6αR)-6-(aminomethyl)-2,2-dimethyl-tetrahydro-3αH-cyclopenta[d][1,3]dioxol-4-yl]-N-[(4-methoxyphenyl)methyl]-N-methylpyrrolo[2,3-d]pyrimidin-4-amine (Intermediate A, 0.33 g, 0.75 mmol, 1.0 eq), in NMP (5.0 mL) was added tert-butyl-N-(3-bromopropyl)-N-(2-phenylethyl)carbamate 58a (387 mg, 1.13 mmol, 1.5 eq), and K₃PO₄ (320 mg, 1.51 mmol, 2.0 eq) and the reaction mixture was stirred at 80° C. or 2 h. The mixture was concentrated under vacuum and purified by reverse flash chromatography [column, C₁₈ silica gel; mobile phase, MeOH in Water/0.05% ammonia water, 50% to 90% gradient in 15 min; detector, UV 220 nm]: to afford tert-butyl-N-[3-({[(3αR,4R,6R,6αS)-6-(4-{[(4-methoxyphenyl)methyl](methyl) amino}pyrrolo[2,3-d]pyrimidin-7-yl)-2,2-dimethyl-tetrahydro-3αH-cyclopenta[d][1,3]dioxol-4-yl]methyl}amino)propyl]-N-(2-phenylethyl)carbamate 67a (260 mg, 49%) as an off-white solid. LCMS [conditions: XBridge C18, 50*3.0 mm, 3.5 μm, Mobile Phase A: Water/5 mM NH₄HCO₃, Mobile Phase B: Methanol, 10%-95% B-5 min, 1.50 mL/min, ES, m/z]: T_{R}=4.19 min; [M+H]⁺: 699.

### tert-Butyl-(3-((((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl)(phenyl)amino)propyl)(phenethyl)carbamate (68a)

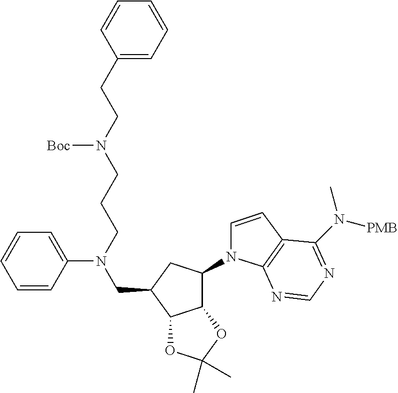

To tert-butyl-N-[3-({[(3αR,4R,6R,6αS)-6-(4-{[(4-methoxyphenyl)methyl](methyl) amino}pyrrolo[2,3-d]pyrimidin-7-yl)-2,2-dimethyl-tetrahydro-3αH-cyclopenta[d][1,3]dioxol-4-yl]methyl}amino)propyl]-N-(2-phenylethyl)carbamate 67a (72 mg, 0.10 mmol, 1.0 eq) in toluene (2.0 mL) was added bromobenzene (49 mg, 0.31 mmol, 3.0 eq), t-BuONa (30 mg, 0.31 mmol, 3.0 eq) and P (t-Bu)₃—PdCl-2nd G (11 mg, 0.02 mmol, 0.2 eq) and the reaction mixture was stirred for 6 h at 80° C. under N₂. The resulting mixture was concentrated under vacuum and the crude residue was purified by silica gel column chromatography (50:50 petroleum ether: AcOEt) to afford tert-butyl-N-[3-({[(3αR,4R,6R,6αS)-6-(4-{[(4-methoxyphenyl)methyl](methyl) amino}pyrrolo[2,3-d]pyrimidin-7-yl)-2,2-dimethyl-tetrahydro-3αH-cyclopenta[d][1,3]dioxol-4-yl]methyl}(phenyl)amino)propyl]-N-(2-phenylethyl)carbamate 68a (61 mg, 76%) as an off-white solid. LCMS [conditions: Kinetex XB-C18, 50*3.0 mm, 2.6 μm, Mobile Phase A: Water/5 mM NH₄HCO₃, Mobile Phase B: Methanol, 10%-95% B-3 min, 1.50 mL/min, ES, m/z]: T_{R}=2.72 min; [M+H]⁺: 775.

### (1R,2S,3R,5R)-3-(4-(Methylamino)-7H-pyrrolo[2,3-d]pyrimidin-7-yl)-5-(((3-(phenethylamino)propyl)(phenyl)amino)methyl)cyclopentane-12-diol (440)

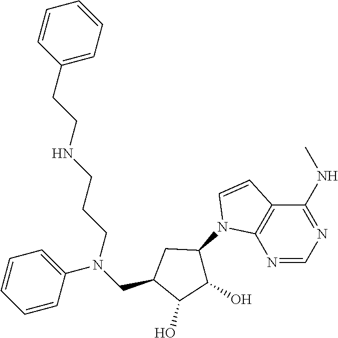 [SMILES: CNc1ncnc2c1ccn2[C@@H]1C[C@H](CN(CCCNCCc2ccccc2)c2ccccc2)[C@@H](O)[C@H]1O]

To tert-butyl-N-[3-({[(3αR,4R,6R,6αS)-6-(4-{[(4-methoxyphenyl)methyl](methyl) amino}pyrrolo[2,3-d]pyrimidin-7-yl)-2,2-dimethyl-tetrahydro-3αH-cyclopenta[d][1,3]dioxol-4-yl]methyl}(phenyl)amino)propyl]-N-(2-phenylethyl)carbamate 68a (59 mg, 0.08 mmol, 1.0 eq) was added trifluoroacetic acid (1.0 mL) at 0° C. and the reaction mixture was stirred for 16 h at room temperature. The resulting mixture was concentrated under vacuum and the residue was dissolved in DMF (2.0 mL) and basified to pH 9-10 with NH₃.H₂O. The crude residue was purified by Prep-HPLC [Column: YMC-Actus Triart C₁₈ ExRS, 30*150 mm, 5 μm; Mobile Phase A: Water (10 mmol/L NH₄HCO₃+0.1% NH₃.H₂O), Mobile Phase B: ACN; Flow rate: 30 ml/min; Gradient: 24% B to 49% B in 7 min, 49% B; Wave Length: 220 nm]: to afford (1R,2S,3R,5R)-3-[4-(methylamino) pyrrolo[2,3-d]pyrimidin-7-yl]-5-{[phenyl({3-[(2-phenylethyl) amino]propyl}) amino]methyl}cyclopentane-1,2-diol 440 (16.2 mg, 41%) as an off-white solid. ¹H NMR (300 MHz, Chloroform-d) δ 8.32 (s, 1H), 7.35-7.31 (m, 1H), 7.30-7.14 (m, 6H), 6.92 (d, J=3.7 Hz, 1H), 6.85-6.71 (m, 3H), 6.36 (d, J=3.6 Hz, 1H), 5.10 (s, 1H), 4.71-4.55 (m, 1H), 4.27 (t, J=7.3 Hz, 1H), 4.09-4.00 (m, 1H), 3.65-3.51 (m, 1H), 3.50-3.36 (m, 2H), 3.33-3.25 (m, 1H), 3.24-3.19 (m, 3H), 2.96-2.78 (m, 4H), 2.76-2.66 (m, 2H), 2.65-2.58 (m, 1H), 2.57-2.46 (m, 1H), 1.88-1.72 (m, 3H). LCMS [conditions: ZORBAX SB-Aq, 50*4.6 mm, 1.8 μm, Mobile Phase A: Water/0.05% TFA, Mobile Phase B: Acetonitrile, 5%-95% B-3 min-1.5, 1.50 mL/min, ES, m/z]: T_{R}=1.32 min; [M+H]⁺: 515. Chiral-SFC [Column: Lux-4 100×4.6 mm 3.0 μm, Solvent B: MeOH (20 mM NH₃), Oven Temperature: 35° C., Total Flow: 3.00 mL/min]: T_{R}=1.48 min; single peak.

### Synthesis of (1S,2R,3R,5R)-3-(((1-methyl-1H-pyrazol-4-yl)(3-(phenethylamino)propyl) amino)methyl)-5-(4-(methylamino)-7H-pyrrolo[2,3-d]pyrimidin-7-yl) cyclopentane-1,2-diol (441)

### 7-((3αS,4R,6R,6αR)-2,2-Dimethyl-6-(((1-methyl-1H-pyrazol-4-yl) amino) methyl)tetrahydro-4H-cyclopenta[d][1,3]dioxol-4-yl)-N-(4-methoxybenzyl)-N-methyl-7H-pyrrolo[2,3-d]pyrimidin-4-amine (70a)

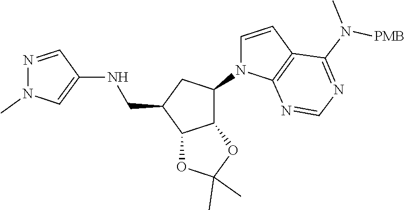

To 1-methylpyrazol-4-amine (67 mg, 0.69 mmol, 2.0 eq) in DCM (5.0 mL) and AcOH (41 mg, 0.69 mmol, 2.0 eq), was added (3αR,4S,6R,6αS)-6-(4-{[(4-methoxyphenyl)methyl](methyl) amino}pyrrolo[2,3-d]pyrimidin-7-yl)-2,2-dimethyl-tetrahydro-3αH-cyclopenta[d][1,3]dioxole-4-carbaldehyde 19a (150 mg, 0.34 mmol, 1.0 eq) and NaBH (OAc)₃ (146 mg, 0.69 mmol, 2.0 eq), at 0° C., and the reaction mixture was stirred at room temperature for 1 h. The resulting mixture was concentrated under vacuum and the crude residue was purified by reverse flash chromatography [column, C₁₈ silica gel; mobile phase, MeOH in Water/0.05% ammonia water, 50% to 90% gradient in 15 min; detector, UV 220 nm]: to afford N-{[(3ªR,4R,6R,6αS)-6-(4-{[(4-methoxyphenyl)methyl](methyl) amino}pyrrolo[2,3-d]pyrimidin-7-yl)-2,2-dimethyl-tetrahydro-3αH-cyclopenta[d][1,3]dioxol-4-yl]methyl}-1-methylpyrazol-4-amine 70a (150 mg, 59%) as a light brown solid. LCMS [conditions: XBridge C18, 50*3.0 mm, 3.5 μm, Mobile Phase A: Water/5 mM NH₄HCO₃, Mobile Phase B: Methanol, 30%-95% B-5 min, 1.50 mL/min, ES, m/z]: T_{R}=2.89 min; [M+H]⁺: 518.

### tert-Butyl-N-(3-oxopropyl)-N-(2-phenylethyl)carbamate (71a)

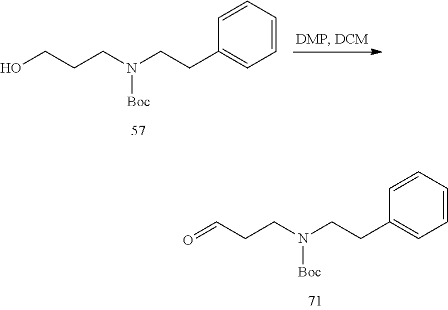 [SMILES: CC(C)(C)OC(=O)N(CCCO)CCc1ccccc1 ; CC(C)(C)OC(=O)N(CCC=O)CCc1ccccc1]

To a solution of tert-butyl-N-(3-hydroxypropyl)-N-(2-phenylethyl)carbamate 57a (2.0 g, 7.16 mmol, 1.0 eq) in DCM (20.0 mL) was added DMP (3.90 g, 9.31 mmol, 1.3 eq) and the reaction mixture was stirred for 1 h at room temperature. The crude residue was purified by silica gel column chromatography (50:50 Petroleum ether: AcOEt) to afford tert-butyl-N-(3-oxopropyl)-N-(2-phenylethyl)carbamate 71a (1.30 g, 65%) as a colorless oil.

### tert-Butyl-(3-((((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl)(1-methyl-1H-pyrazol-4-yl) amino)propyl)(phenethyl)carbamate (72a)

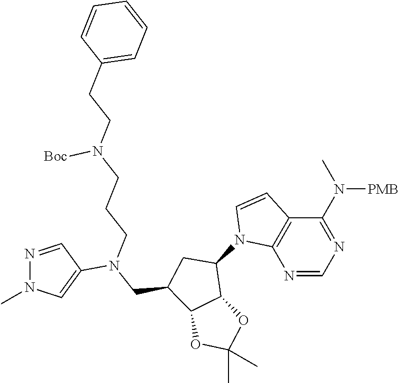

To N-{[(3αR,4R,6R,6αS)-6-(4-{[(4-methoxyphenyl)methyl](methyl) amino}pyrrolo[2,3-d]pyrimidin-7-yl)-2,2-dimethyl-tetrahydro-3αH-cyclopenta[d][1,3]dioxol-4-yl]methyl}-1-methylpyrazol-4-amine 70a (150 mg, 0.20 mmol, 1.0 eq) in DCM (3.0 mL) and AcOH (24 mg, 0.41 mmol, 2.0 eq) were added tert-butyl-N-(3-oxopropyl)-N-(2-phenylethyl)carbamate 71a (84 mg, 0.30 mmol, 1.5 eq) and NaBH (OAc)₃ (86 mg, 0.41 mmol, 2.0 eq), at 0° C., and the reaction mixture was stirred at room temperature for 1 h. The resulting mixture was concentrated under vacuum and the crude residue was purified by reverse flash chromatography [column, C₁₈ silica gel; mobile phase, MeOH in Water/0.05% ammonia water, 50% to 90% gradient in 15 min; detector, UV 220 nm)]: to afford tert-butyl-N-[3-({[(3αR,4R,6R,6αS)-6-(4-{[(4-methoxyphenyl)methyl](methyl) amino}pyrrolo[2,3-d]pyrimidin-7-yl)-2,2-dimethyl-tetrahydro-3αH-cyclopenta[d][1,3]dioxol-4-yl]methyl}(1-methylpyrazol-4-yl) amino)propyl]-N-(2-phenylethyl)carbamate 72a (120 mg, 76%) as a light brown solid. LCMS [conditions: XBridge C18, 50*3.0 mm, 3.5 μm, Mobile Phase A: Water/5 mM NH₄HCO₃, Mobile Phase B: Methanol, 30%-95% B-5 min, 1.50 mL/min, ES, m/z]: T_{R}=4.08 min; [M+H]⁺: 779.

### (1S,2R,3R,5R)-3-(((1-Methyl-1H-pyrazol-4-yl)(3-(phenethylamino)propyl) amino)methyl)-5-(4-(methylamino)-7H-pyrrolo[2,3-d]pyrimidin-7-yl) cyclopentane-1,2-diol (441)

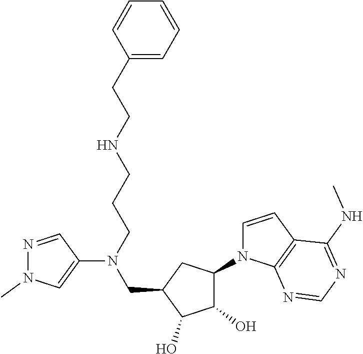 [SMILES: CNc1ncnc2c1ccn2[C@@H]1C[C@H](CN(CCCNCCc2ccccc2)c2cnn(C)c2)[C@@H](O)[C@H]1O]

To tert-butyl-N-[3-({[(3αR,4R,6R,6αS)-6-(4-{[(4-methoxyphenyl)methyl](methyl) amino}pyrrolo[2,3-d]pyrimidin-7-yl)-2,2-dimethyl-tetrahydro-3αH-cyclopenta[d][1,3]dioxol-4-yl]methyl}(1-methylpyrazol-4-yl) amino)propyl]-N-(2-phenylethyl)carbamate 72a (60 mg, 0.08 mmol, 1.0 eq) was added trifluoroacetic acid (2.0 mL) at 0° C. and the reaction mixture was stirred at room temperature for 16 h. The resulting mixture was concentrated under vacuum, the residue was dissolved in DMF (2.0 mL) and basified to pH 9-10 with NH₃.H₂O. The crude product was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 ml/min; Gradient: 24% B to 49% B in 9 min, 49% B; Wave Length: 220 nm]: to afford (1R,2S,3R,5R)-3-[4-(methylamino) pyrrolo[2,3-d]pyrimidin-7-yl]-5-{[(1-methylpyrazol-4-yl)({3-[(2-phenylethyl) amino]propyl}) amino]methyl}cyclopentane-1,2-diol 441 (10.9 mg, 27%) as an off-white solid. ¹H NMR (300 MHZ, Chloroform-d) 8 8.32 (s, 1H), 7.36-7.30 (m, 2H), 7.25-7.18 (m, 4H), 7.05-6.98 (m, 1H), 6.95 (d, J=3.6 Hz, 1H), 6.37 (d, J=3.6 Hz, 1H), 5.12 (d, J=5.8 Hz, 1H), 4.76-4.60 (m, 1H), 4.29-4.19 (m, 1H), 4.10-3.99 (m, 1H), 3.84 (s, 3H), 3.22 (d, J=5.0 Hz, 3H), 3.18-3.01 (m, 3H), 2.99-2.82 (m, 5H), 2.81-2.65 (m, 2H), 2.56-2.37 (m, 2H), 1.86-1.65 (m, 3H). LCMS [conditions: ZORBAX SB-Aq, 50*4.6 mm, 1.8 μm, Mobile Phase A: Water/0.05% TFA, Mobile Phase B: Acetonitrile, 5%-95% B-3 min-1.5, 1.50 mL/min, ES, m/z]: T_{R}=1.13 min; [M+H]⁺: 519. Chiral-SFC [Column: Lux-4 100×4.6 mm 3.0 μm, Solvent B: MeOH (20 mM NH₃), Oven Temperature: 35° C., Total Flow: 3.00 mL/min]: T_{R}=1.09 min; single peak.

### Synthesis of (1R,2S,3R,5R)-3-(4-(methylamino)-7H-pyrrolo[2,3-d]pyrimidin-7-yl)-5-(((3-(phenethylamino)propyl)(1H-pyrazol-4-yl) amino) methyl) cyclopentane-1,2-diol (442)

### Synthesis of 1-((2-(trimethylsilyl) ethoxy)methyl)-1H-pyrazol-4-amine 4-Nitro-1-((2-(trimethylsilyl) ethoxy)methyl)-1H-pyrazole (76a)

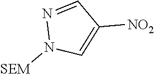 [SMILES: C[Si](C)(C)CCOCn1cc([N+](=O)[O-])cn1]

To 4-nitropyrazole (0.60 g, 5.3 mmol, 1.0 eq) in THF (10.0 mL) was added NaH (0.64 g, 15.9 mmol, 3.0 eq) in several portions at 0° C. under N₂ and the reaction mixture was stirred at room temperature for 30 min. The resulting mixture was cooled to 0° C. and then SEMCl (1.77 g, 10.6 mmol, 2.0 eq) was added and the reaction mixture was stirred at room temperature for 1 h. The reaction was quenched by the addition of H₂O (20.0 mL) at 0° C. and the resulting mixture was extracted with AcOEt (2×20 mL). The combined organic layers were dried over anhydrous Na₂SO₄, filtered and then the filtrate was concentrated under reduced pressure. The residue was purified by silica gel column chromatography to afford 4-nitro-1-{[2-(trimethylsilyl) ethoxy]methyl}pyrazole 76a (1.20 g, 93%) as a colorless oil.

### 1-((2-(Trimethylsilyl) ethoxy)methyl)-1H-pyrazol-4-amine (75a)

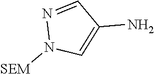 [SMILES: C[Si](C)(C)CCOCn1cc(N)cn1]

To 4-nitro-1-{[2-(trimethylsilyl) ethoxy]methyl}pyrazole 76a (1.20 g, 4.90 mmol, 1.0 eq) in AcOEt (20.0 mL) was added Pd/C(0.20 g, 1.90 mmol, 0.4 eq) and the resulting mixture was stirred for 2 h at room temperature under a H₂ baloon. The resulting mixture was filtered and the filtrate was concentrated under reduced pressure to afford 1-{[2-(trimethylsilyl) ethoxy]methyl}pyrazol-4-amine 75a (1.05 g, 99%) as a light brown oil. LCMS [Poroshell HPH-C18, 50*3.0 mm, 2.7 μm; Mobile Phase A: Water/5 mM NH₄HCO₃, Mobile Phase B: MeOH, 10%-95% B-5 min (+−), 1.50 mL/min, ES, m/z]: T_{R}=2.98 min; [M+H]⁺: 214.

### 7-((3αS,4R,6R,6αR)-2,2-Dimethyl-6-(((1-((2-(trimethylsilyl) ethoxy)methyl)-1H-pyrazol-4-yl) amino) methyl)tetrahydro-4H-cyclopenta[d][1,3]dioxol-4-yl)-N-(4-methoxybenzyl)-N-methyl-7H-pyrrolo[2,3-d]pyrimidin-4-amine (73a)

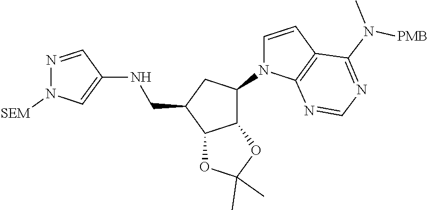

To 1-{[2-(trimethylsilyl) ethoxy]methyl}pyrazol-4-amine 75a (73 mg, 0.30 mmol, 1.5 eq) in DCM (5.0 mL) and acetic acid (27 mg, 0.5 mmol, 2.0 eq), at 0° C., were added (3αR,4S,6R,6αS)-6-(4-{[(4-methoxyphenyl)methyl](methyl) amino}pyrrolo[2,3-d]pyrimidin-7-yl)-2,2-dimethyl-tetrahydro-3αH-cyclopenta[d][1,3]dioxole-4-carbaldehyde 19a (100 mg, 0.20 mmol, 1.0 eq) and NaBH (OAc)₃ (97 mg, 0.5 mmol, 2.0 eq) and the reaction mixture was stirred at room temperature for 1 h. The resulting mixture was concentrated under vacuum and the crude residue was purified by reverse phase flash [column, C₁₈ silica gel; mobile phase, CH₃CN in Water/0.05% ammonia water, 20% to 60% gradient in 15 min; detector, UV 220 nm)]: to afford N-{[(3αR,4R,6R,6αS)-6-(4-{[(4-methoxyphenyl)methyl](methyl) amino}pyrrolo[2,3-d]pyrimidin-7-yl)-2,2-dimethyl-tetrahydro-3αH-cyclopenta[d][1,3]dioxol-4-yl]methyl}-1-{[2-(trimethylsilyl) ethoxy]methyl}pyrazol-4-amine 73a (90 mg, 62%) as an off-white solid. LCMS [XBridge C18, 50*3.0 mm, 3.5 μm; Mobile Phase A: Water/5 mM NH₄HCO₃, Mobile Phase B: MeOH, 10%-95% B (2 min)-4 min (+), 1.00 mL/min, ES, m/z]: T_{R}=2.36 min; [M+H]⁺: 634.

### tert-Butyl-(3-((((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl)(1-((2-(trimethylsilyl) ethoxy)methyl)-1H-pyrazol-4-yllamino\nronyl)(nhenethyl)carbamate (74a)

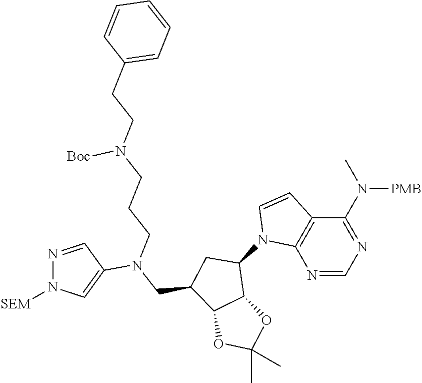

To tert-butyl-N-(3-oxopropyl)-N-(2-phenylethyl)carbamate 71a (31 mg, 0.10 mmol, 1.2 eq) in DCM (3.0 mL) was added acetic acid (56 mg, 0.10 mmol, 10.0 eq) at room temperature and the resulting mixture was stirred for 30 min at room temperature. After this time N-{[(3αR,4R,6R,6αS)-6-(4-{[(4-methoxyphenyl)methyl](methyl) amino}pyrrolo[2,3-d]pyrimidin-7-yl)-2,2-dimethyl-tetrahydro-3αH-cyclopenta[d][1,3]dioxol-4-yl]methyl}-1-{[2-(trimethylsilyl) ethoxy]methyl}pyrazol-4-amine 73a (60 mg, 0.10 mmol, 1.0 eq) in DCM (1.0 mL) was added and the resulting mixture was stirred for 1 h at room temperature. Then Boc₂O (31 mg, 0.10 mmol, 1.5 eq) and TEA (144 mg, 1.4 mmol, 15.0 eq) were added and the reaction mixture was stirred for 30 min at room temperature. The resulting mixture was concentrated under reduced pressure and the crude product was purified by HPLC [Column: Xtimate C18 50×250 mm, 10 μm; Mobile Phase A: Water (0.1% NH₃.H₂O), Mobile Phase B: ACN; Flow rate: 90 mL/min; Gradient: 75% B to 90% B in 12 min, 90% B; Wave Length: 220 nm]: to afford tert-butyl-N-[3-({[(3αR,4R,6R,6αS)-6-(4-{[(4-methoxyphenyl)methyl](methyl) amino}pyrrolo[2,3-d]pyrimidin-7-yl)-2,2-dimethyl-tetrahydro-3αH-cyclopenta[d][1,3]dioxol-4-yl]methyl}(1-{[2-(trimethylsilyl) ethoxy]methyl}pyrazol-4-yl) amino)propyl]-N-(2-phenylethyl)carbamate 74a (30 mg, 35%) as a yellow solid. LCMS [Poroshell HPH-C18, 50*3.0 mm, 2.7 μm; Mobile Phase A: Water/5 mM NH₄HCO₃, Mobile Phase B: MeOH, 10%-95% B-5 min (+−), 1.50 mL/min, ES, m/z]: T_{R}=4.54 min; [M+H]⁺: 895.

### (1R,2S,3R,5R)-3-(4-(Methylamino)-7H-pyrrolo[2,3-d]pyrimidin-7-yl)-5-(((3-(phenethylamino)propyl)(1H-pyrazol-4-yl) amino) methyl) cyclopentane-1,2-diol (442)

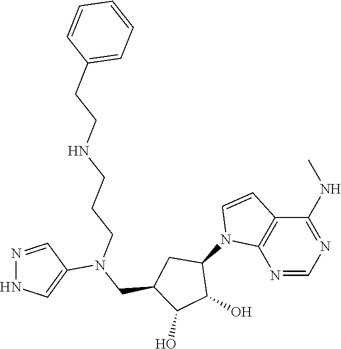 [SMILES: CNc1ncnc2c1ccn2[C@@H]1C[C@H](CN(CCCNCCc2ccccc2)c2cn[nH]c2)[C@@H](O)[C@H]1O]

To tert-butyl-N-[3-({[(3αR,4R,6R,6αS)-6-(4-{[(4-methoxyphenyl)methyl](methyl) amino}pyrrolo[2,3-d]pyrimidin-7-yl)-2,2-dimethyl-tetrahydro-3αH-cyclopenta[d][1,3]dioxol-4-yl]methyl}(1-{[2-(trimethylsilyl) ethoxy]methyl}-pyrazol-4-yl) amino)propyl]-N-(2-phenylethyl)carbamate 74a (30 mg, 0.03 mmol, 1.0 eq) was added TFA (1.0 mL), at room temperature, and the reaction mixture was stirred at 50° C. for 2 h. The resulting mixture was concentrated under reduced pressure and the residue was basified to pH 10 with NH₃.H₂O. The crude product was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 20% B to 55% B in 7 min, 55% B; Wave Length: 220 nm]: to afford (1R,2S,3R,5R)-3-[4-(methylamino) pyrrolo[2,3-d]pyrimidin-7-yl]-5-[{{3-[(2-phenylethyl) amino]propyl}(1H-pyrazol-4-yl) amino) methyl]cyclopentane-1,2-diol 442 (10.5 mg, 62%) as an off-white solid. ¹H NMR (400 MHZ, DMSO-d₆) δ 12.16 (s, 1H), 8.12 (s, 1H), 7.41-7.32 (m, 1H), 7.29-7.21 (m, 3H), 7.20-7.12 (m, 5H), 6.56-6.49 (m, 1H), 4.88-4.74 (m, 2H), 4.64 (s, 1H), 4.34-4.22 (m, 1H), 3.81-3.73 (m, 1H), 3.20-3.10 (m, 1H), 3.07-2.98 (m, 2H), 2.97-2.87 (m, 4H), 2.74-2.63 (m, 4H), 2.57-2.52 (m, 2H), 2.26-2.07 (m, 2H), 1.63-1.42 (m, 3H). LCMS [conditions ZORBAX SB-Aq, 50*4.6 mm, 1.8 μm, Mobile Phase A: Water/0.02% TFA, Mobile Phase B: Acetonitrile, 5%-95% B-3 min, 1.50 mL/min, ES, m/z]: T_{R}=1.08 min; [M+H]⁺: 505. Chiral-SFC [Column: SB 100×4.6 mm 3.0 μm, Solvent B: MeOH (20 mM NH₃), Oven Temperature: 35° C., Total Flow: 3.00 mL/min]: T_{R}=1.18 min; single peak.

### Synthesis of (1R,2S,3R,5R)-3-[4-(methylamino) pyrrolo[2,3-d]pyrimidin-7-yl]-5-{[(1-methylpyrazol-3-yl)({3-[(2-phenylethyl) amino]propyl}) amino]methyl}cyclopentane-1,2-diol (443)

### Synthesis of tert-butyl-N-{3-[(1-methylpyrazol-3-yl) amino]propyl}-N-(2-phenylethyl)carbamate (83a)

### tert-Butyl-N-(3-oxopropyl)-N-(2-phenylethyl)carbamate (71a)

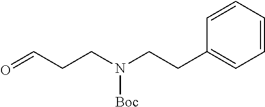 [SMILES: CC(C)(C)OC(=O)N(CCC=O)CCc1ccccc1]

To tert-butyl-N-(3-hydroxypropyl)-N-(2-phenylethyl)carbamate 57a (2.00 g, 7.16 mmol, 1.0 eq) in DCM (20 mL) was added DMP (3.90 g, 9.31 mmol, 1.3 eq) and the reaction mixture was stirred for 1 h at room temperature. The crude residue was purified by silica gel column chromatography (50:50 Petroleum ether: AcOEt) to afford tert-butyl-N-(3-oxopropyl)-N-(2-phenylethyl)carbamate 71a (1.30 g, 65%) as a colorless oil.

### tert-Butyl-N-{3-[(1-methylpyrazol-3-yl) amino]propyl}-N-(2-phenylethyl)carbamate (83a)

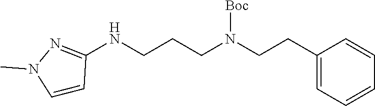 [SMILES: Cn1ccc(NCCCN(CCc2ccccc2)C(=O)OC(C)(C)C)n1]

To a solution of 1-methylpyrazol-3-amine (0.13 g, 1.30 mmol, 1.5 eq) in DCM (1.0 mL) was added AcOH (0.05 g, 0.9 mmol, 1.0 eq) and NaBH (OAc)₃ (0.38 mg, 1.8 mmol, 2.0 eq) and the reaction mixture was stirred for 30 min at 0° C. To the resulting mixture was added tert-butyl-N-(3-oxopropyl)-N-(2-phenylethyl)carbamate 71 (0.25 g, 0.90 mmol, 1.0 eq) in DCM (1.0 mL), dropwise, over 3 min, at 0° C. and the reaction mixture was stirred for additional 30 min at 0° C. The crude residue was purified by silica gel column chromatography (95:5 DCM: MeOH) to afford tert-butyl-N-{3-[(1-methylpyrazol-3-yl) amino]propyl}-N-(2-phenylethyl)carbamate 83a (0.20 g, 62%) as a light yellow oil. LCMS [conditions Xbridge Shield C18, 50*3.0 mm, 3.5 μm, Mobile Phase A: Water/0.05% Ammonia, Mobile Phase B: Acetonitrile, 5%-100% B-5 min (+), 1.50 L/min, ES, m/z]: T_{R}=3.61 min; [M+H]⁺: 359

### tert-Butyl-N-[3-({[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}(1-methylpyrazol-3-yl) amino)propyl]-N-(2-phenylethyl)carbamate (84a)

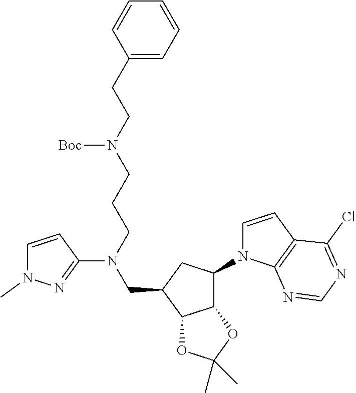 [SMILES: Cn1ccc(N(CCCN(CCc2ccccc2)C(=O)OC(C)(C)C)C[C@H]2C[C@@H](n3ccc4c(Cl)ncnc43)[C@@H]3OC(C)(C)O[C@H]23)n1]

To tert-butyl-N-{3-[(1-methylpyrazol-3-yl) amino]propyl}-N-(2-phenylethyl)carbamate 83a (0.20 g, 0.60 mmol, 1.5 eq) in DCM (1.0 mL) at 0° C. was added AcOH (0.44 g, 7.40 mmol, 20 eq) and NaBH (OAc)₃ (0.16 g, 0.70 mmol, 2.0 eq) and the reaction mixture was stirred for 30 min at 0° C. To the resultant mixture was added [(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methanol 5a (0.12 g, 0.37 mmol, 1.0 eq) in DCM (1.0 mL), dropwise over 3 min, at 0° C., and the reaction mixture was stirred for additional 30 min at 0° C. The resulting mixture was concentrated under vacuum, the residue was dissolved in MeOH (2.0 ml) and the crude residue was purified by Prep-HPLC [Column: Xtimate C18 50×250 mm, 10 μm; Mobile Phase A: Water (0.1% NH₃.H₂O), Mobile Phase B: ACN; Flow rate: 90 mL/min; Gradient: 45% B to 75% B in 12 min; Wave Length: 220 nm]: to afford tert-butyl-N-[3-({[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}(1-methylpyrazol-3-yl) amino)propyl]-N-(2-phenylethyl)carbamate 84a (0.12 g, 49%) as a light yellow oil. LCMS [conditions Xbridge Shield C18, 50*3.0 mm, 3.5 μm, Mobile Phase A: Water/0.05% Ammonia, Mobile Phase B: Acetonitrile, 10%-95% B-5 min (+), 1.50 L/min, ES, m/z]: T_{R}=4.16 min; [M+H]⁺: 664.

### tert-Butyl-N-[3-({[(3αR,4R,6R,6αS)-2,2-dimethyl-6-[4-(methylamino) pyrrolo[2,3-d]pyrimidin-7-yl]-tetrahydro-3αH-cyclopenta[d][1,3]dioxol-4-yl]methyl}(1-methylpyrazol-3-yl) amino)propyl]-N-(2-phenylethyl)carbamate (85a)

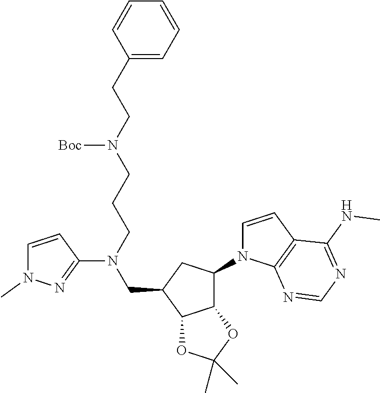

To tert-butyl-N-[3-({[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}(1-methylpyrazol-3-yl) amino)propyl]-N-(2-phenylethyl)carbamate 84a (0.12 g, 0.20 mmol, 1.0 eq) was added methylamine (30% in ethanol, 2.0 ml) and the reaction mixture was stirred at 80° C. for 1 h. The resulting mixture was concentrated under vacuum to afford tert-butyl-N-[3-({[(3αR,4R,6R,6αS)-2,2-dimethyl-6-[4-(methylamino) pyrrolo[2,3-d]pyrimidin-7-yl]-tetrahydro-3αH-cyclopenta[d][1,3]dioxol-4-yl]methyl}(1-methylpyrazol-3-yl) amino)propyl]-N-(2-phenylethyl)carbamate 85a (0.12 g, crude) as a light yellow solid. The crude product was used in the next step directly without further purification. LCMS [conditions Xbridge Shield C18, 50*3.0 mm, 3.5 μm, Mobile Phase A: Water/0.05% Ammonia, Mobile Phase B: Acetonitrile, 5%-100% B-3 min (+), 1.50 L/min, ES, m/z]: T_{R}=1.83 min; [M+H]⁺: 659

### (1R,2S,3R,5R)-3-[4-(Methylamino) pyrrolo[2,3-d]pyrimidin-7-yl]-5-{[(1-methylpyrazol-3-yl)({3-[(2-phenylethyl) amino]propyl}) amino]methyl}cyclopentane-1,2-diol (443)

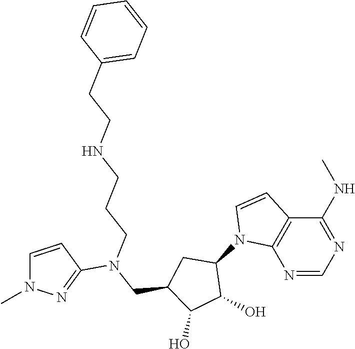 [SMILES: CNc1ncnc2c1ccn2[C@@H]1C[C@H](CN(CCCNCCc2ccccc2)c2ccn(C)n2)[C@@H](O)[C@H]1O]

To tert-butyl-N-[3-({[(3αR,4R,6R,6αS)-2,2-dimethyl-6-[4-(methylamino) pyrrolo[2,3-d]pyrimidin-7-yl]-tetrahydro-3αH-cyclopenta[d][1,3]dioxol-4-yl]methyl}(1-methylpyrazol-3-yl) amino)propyl]-N-(2-phenylethyl)carbamate 85a (0.12 g, 0.20 mmol, 1.0 eq) was added HCl (4M in 1,4-dioxane, 2.0 mL) at room temperature and the resulting mixture was stirred for 1 h at room temperature and concentrated under vacuum. The resultant residue was dissolved in H₂O (2.0 mL) and the mixture was neutralized to pH>7 with NH₃.H₂O. The crude residue was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 24% B to 50% B in 7 min, 50% B; Wave Length: 220 nm]: to afford (1R,2S,3R,5R)-3-[4-(methylamino) pyrrolo[2,3-d]pyrimidin-7-yl]-5-{[(1-methylpyrazol-3-yl)({3-[(2-phenylethyl) amino]propyl}) amino]methyl}cyclopentane-1,2-diol 443 (45.8 mg, 48%) as an off-white solid. ¹H NMR (300 MHZ, DMSO-d₆+D₂O) 88.08 (s, 1H), 7.35-7.30 (m, 1H), 7.29-7.21 (m, 3H), 7.20-7.11 (m, 3H), 6.52 (d, J=3.5 Hz, 1H), 5.56-5.50 (m, 1H), 4.88-4.75 (m, 1H), 4.30-4.20 (m, 1H), 3.79-3.74 (m, 1H), 3.58 (s, 3H), 3.38-3.25 (m, 1H), 3.23-3.07 (m, 3H), 2.92 (s, 3H), 2.67 (s, 3H), 2.49-2.42 (m, 3H), 2.33-2.21 (m, 1H), 2.20-2.01 (m, 1H), 1.68-1.55 (m, 2H), 1.54-1.39 (m, 1H). LCMS [conditions Cortecs C_{18+, 50*3.0} mm, 2.7 μm, Mobile Phase A: Water/0.02% TFA, Mobile Phase B: Acetonitrile, 5%-95% B-3 min, 1.50 mL/min, ES, m/z]: T_{R}=0.70 min; [M+H]⁺: 519 Chiral-SFC [Column: IG 100×4.6 mm 3.0 μm, Solvent B: MeOH (20 mM NH₃), Oven Temperature: 35° C., Total Flow: 3.00 mL/min]: T_{R}=2.72 min; single peak.

### Synthesis of ((1R,2S,3R,5R)-3-[4-(methylamino) pyrrolo[2,3-d]pyrimidin-7-yl]-5-[({3-[(2-phenylethyl) amino]propyl}(1H-pyrazol-3-yl) amino) methyl]cyclopentane-1,2-diol (444)

### tert-Butyl-N-(2-phenylethyl)-N-[3-(1H-pyrazol-3-ylamino)propyl]carbamate (86a)

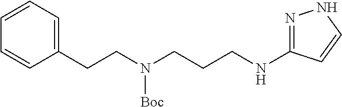 [SMILES: CC(C)(C)OC(=O)N(CCCNc1cc[nH]n1)CCc1ccccc1]

To a solution of 1H-pyrazol-3-amine (0.11 g, 1.30 mmol, 1.5 eq) in DCM (1.0 mL), at 0° C., was added AcOH (0.05 g, 0.90 mmol, 1.0 eq) and NaBH (OAc)₃ (0.38 g, 1.80 mmol, 2.0 eq), and the resulting mixture was stirred for 30 min at 0° C. Then tert-butyl-N-(3-oxopropyl)-N-(2-phenylethyl)carbamate 71a (0.25 g, 0.9 mmol, 1.0 eq) in DCM (1.0 mL) was added dropwise over 3 min at 0° C. and the reaction mixture was stirred for 30 min at 0° C. The crude residue was purified by silica gel column chromatography (96:4 DCM: MeOH) to afford tert-butyl-N-(2-phenylethyl)-N-[3-(1H-pyrazol-3-ylamino)propyl]carbamate 86a (0.20 g, 64%) as a light yellow oil. LCMS [conditions HPH-C18, 30*3.0 mm, 1.9 μm, Mobile Phase A: Water/0.05% Ammonia, Mobile Phase B: Acetonitrile, 5%-100% B-1.2 min (+)(+), 1.50 L/min, ES, m/z]: T_{R}=0.81 min; [M+H]⁺: 345.

### tert-Butyl-N-[3-({[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}(1H-pyrazol-3-yl) amino)propyl]-N-(2-phenylethyl)carbamate (87a)

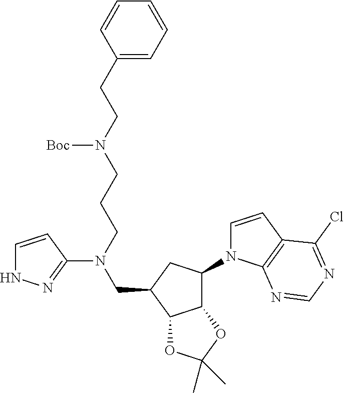 [SMILES: CC(C)(C)OC(=O)N(CCCN(C[C@H]1C[C@@H](n2ccc3c(Cl)ncnc32)[C@@H]2OC(C)(C)O[C@H]12)c1cc[nH]n1)CCc1ccccc1]

To a solution of tert-butyl-N-(2-phenylethyl)-N-[3-(1H-pyrazol-3-ylamino)propyl]carbamate 86a (0.29 g, 0.80 mmol, 1.5 eq) in DCM (1.0 mL), at 0° C., was added AcOH (0.67 g, 11.1 mmol, 20 eq) and NaBH (OAc)₃ (0.23 g, 1.10 mmol, 2.0 eq) and the resulting mixture was stirred for 30 min at 0° C. A solution of (3αR,4S,6R,6αS)-6-{4- chloropyrrolo[2,3-d]pyrimidin-7-yl}-2,2-dimethyl-tetrahydro-3αH-cyclopenta[d][1,3]dioxole-4-carbaldehyde 77a (0.18 g, 0.60 mmol, 1.0 eq) in DCM (1.0 mL) was then added, dropwise over 3 min at 0° C. and the reaction mixture was stirred for 30 min at 0° C. The resulting mixture was concentrated under vacuum and the crude residue was dissolved in MeOH (2.0 ml) and purified by Prep-HPLC [Column: Xtimate C18 50×250 mm, 10 μm; Mobile Phase A: Water (0.1% NH₃.H₂O), Mobile Phase B: ACN; Flow rate: 90 mL/min; Gradient: 35% B to 60% B in 12 min, 60% B; Wave Length: 220 nm]: to afford tert-butyl-N-[3-({[(3ªR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}(1H-pyrazol-3-yl) amino)propyl]-N-(2-phenylethyl)carbamate 87a (0.09 g, 25%) as a light yellow oil. LCMS [conditions XBridge C18, 50*3.0 mm, 3.5 μm, Mobile Phase A: Water/5 mM NH₄HCO₃, Mobile Phase B: Methanol, 10%-95% B-5 min (+), 1.50 L/min, ES, m/z, ES, m/z]: T_{R}=4.12 min; [M+H]⁺: 650

### tert-Butyl-NV-[3-({[(3αR,4R,6R,6αS)-2,2-dimethyl-6-[4-(methylamino) pyrrolo[2,3-d]pyrimidin-7-yl]-tetrahydro-3αH-cyclopenta[d][1,3]dioxol-4-yl]methyl}(1H-pyrazol-3-yl) amino)propyl]-N-(2-phenylethyl)carbamate (88a)

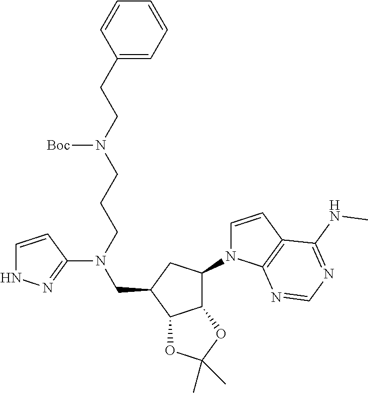 [SMILES: CNc1ncnc2c1ccn2[C@@H]1C[C@H](CN(CCCN(CCc2ccccc2)C(=O)OC(C)(C)C)c2cc[nH]n2)[C@H]2OC(C)(C)O[C@H]21]

To tert-butyl-N-[3-({[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}(1H-pyrazol-3-yl) amino)propyl]-N-(2-phenylethyl)carbamate 87a (90 mg, 0.14 mmol, 1.0 eq) was added methylamine solution (30% in EtOH, 2.0 mL) and the resulting mixture was stirred at 80° C. for 1 h. The resulting mixture was concentrated under vacuum to afford tert-butyl-N-[3-({[(3αR,4R,6R,6αS)-2,2-dimethyl-6-[4-(methylamino) pyrrolo[2,3-d]pyrimidin-7-yl]-tetrahydro-3αH-cyclopenta[d][1,3]dioxol-4-yl]methyl}(1H-pyrazol-3-yl) amino)propyl]-N-(2-phenylethyl)carbamate 88a (90 mg, crude) as a light yellow solid. The crude product was used in the next step directly without further purification. LCMS [conditions XBridge C18, 50*3.0 mm, 3.5 μm, Mobile Phase A: Water/5 mM NH₄HCO₃, Mobile Phase B: Methanol, 10%-95% B-3 min (+), 1.50 L/min, ES, m/z, ES, m/z]: T_{R}=2.27 min; [M+H]⁺: 645.

### ((1R,2S,3R,5R)-3-[4-(Methylamino) pyrrolo[2,3-d]pyrimidin-7-yl]-5-[{{3-[(2-phenylethyl) amino]propyl}(1H-pyrazol-3-yl) amino) methyl]cyclopentane-1,2-diol (444)

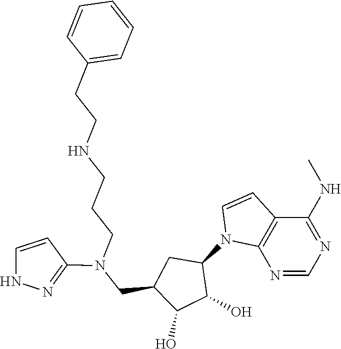 [SMILES: CNc1ncnc2c1ccn2[C@@H]1C[C@H](CN(CCCNCCc2ccccc2)c2cc[nH]n2)[C@@H](O)[C@H]1O]

To tert-butyl-N-[3-({[(3αR,4R,6R,6αS)-2,2-dimethyl-6-[4-(methylamino) pyrrolo[2,3-d]pyrimidin-7-yl]-tetrahydro-3αH-cyclopenta[d][1,3]dioxol-4-yl]methyl}(1H-pyrazol-3-yl) amino)propyl]-N-(2-phenylethyl)carbamate 88a (90 mg, 0.20 mmol, 1.0 eq) was added HCl (4M in dioxane, 2.0 mL) at room temperature and the reaction mixture was stirred for 1 h at room temperature. The resultant mixture was concentrated under vacuum and then dissolved in H₂O (2.0 mL) and neutralized to pH>7 with NH₃.H₂O. The crude residue was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 24% B to 51% B in 8 min, 51% B; Wave Length: 220 nm]: to afford ((1R,2S,3R,5R)-3-[4-(methylamino) pyrrolo[2,3-d]pyrimidin-7-yl]-5-[{{3-[(2-phenylethyl) amino]propyl}(1H-pyrazol-3-yl) amino) methyl]cyclopentane-1,2-diol 444 (45.3 mg, 64%) as an off-white solid. ¹H NMR (300 MHZ, DMSO-d₆+D₂O) 8 8.08 (s, 1H), 7.39-7.31 (m, 1H), 7.30-7.20 (m, 3H), 7.19-7.11 (m, 3H), 6.52 (d, J=3.6 Hz, 1H), 5.63-5.52 (m, 1H), 4.87-4.72 (m, 1H), 4.30-4.20 (m, 1H), 3.77-3.69 (m, 1H), 3.41-3.27 (m, 1H), 3.25-3.07 (m, 3H), 2.92 (s, 3H), 2.78-2.67 (m, 4H), 2.51-2.43 (m, 2H), 2.35-2.22 (m, 1H), 2.20-2.06 (m, 1H), 1.72-1.56 (m, 2H), 1.54-1.37 (m, 1H). LCMS [conditions ZORBAX SB-Aq, 50*4.6 mm, 1.8 μm, Mobile Phase A: Water/0.02% TFA, Mobile Phase B: Acetonitrile, 5%-(20%-50%)-95% B-5 min, 1.50 mL/min, ES, m/z]: T_{R}=1.25 min; [M+H]⁺: 505. Chiral-SFC [Column: Lux-4 100×4.6 mm 3.0 μm, Solvent B: MeOH (20 mM NH₃), Oven Temperature: 35° C., Total Flow: 3.00 mL/min]: T_{R}=1.77 min; single peak.

### Synthesis of (1S,2R,3R,5R)-3-((1,6-diazaspiro[3.3]heptan-1-yl)methyl)-5-(4-(methylamino)-7H-pyrrolo[2,3-d]pyrimidin-7-yl) cyclopentane-1,2-diol (445)

### tert-Butyl-1-(((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl)-1,6-diazaspiro[3.3]heptane-6-carboxylate (180a)

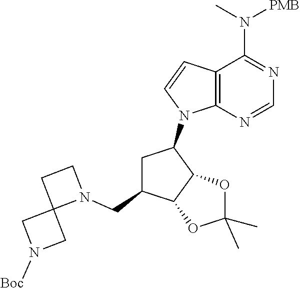

To a stirred solution of (3αR,4S,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxole-4-carbaldehyde 19a (70 mg, 0.16 mmol, 1.0 eq) and 3,6-diazaspiro[3.3]heptane-6-carboxylic acid tert-butyl ester hemioxylate (39 mg, 0.08 mmol, 0.5 eq) in DCM (1.0 mL) were added TEA (16 mq, 0.16 mmol, 1.0 eq) and NaBH(OAc)₃ (51 mq, 0.24 mmol, 1.5 eq) at room temperature and the reaction mixture was stirred for 1 h at room temperature. The resulting mixture was basified to pH 8-9 with 10% NaHCO₃ solution and extracted with AcOEt. The organic layer was washed with brine, dried over anhydrous Na₂SO₄ and filterd. The filtrate was concentrated under reduced pressure and the crude residue was purified by silica gel column chromatography (AcOEt) to afford tert-butyl-1-(((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl)-1,6-diazaspiro[3.3]heptane-6-carboxylate 180a (75 mg, 76%) as a light yellow solid. LCMS [XBridge C18, 50*3.0 mm, 3.5 μm; Mobile Phase A: Water/5 mM NH₄HCO₃, Mobile Phase B: MeOH, 10%-95% B-3 min (+), 1.50 mL/min, ES, m/z]: T_{R}=2.02 min; [M+H]⁺: 619.

### (1S,2R,3R,5R)-3-((1,6-Diazaspiro[3.3]heptan-1-yl)methyl)-5-(4-(methylamino)-7H-pyrrolo[2,3-d]pyrimidin-7-yl) cyclopentane-1,2-diol (445)

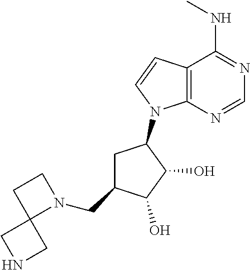 [SMILES: CNc1ncnc2c1ccn2[C@@H]1C[C@H](CN2CCC23CNC3)[C@@H](O)[C@H]1O]

A solution of tert-butyl-1-(((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl)-1,6-diazaspiro[3.3]heptane-6-carboxylate 180a (75 mg, 0.12 mmol, 1.0 eq) in TFA (1.0 mL) was stirred for 16 h at room temperature. The resulting mixture was concentrated under reduced pressure and the crude product was purified by Prep-HPLC [Column, XBridge Shield RP18 OBD Column, 19*150 mm, 5 μm; mobile phase, ACN and Water (10 mmol/L NH₄HCO₃+0.1% NH₃.H₂O) (18% up to 37% in 8 min); Detector, UV 220 nm]: to afford (1S,2R,3R,5R)-3-((1,6-diazaspiro[3.3]heptan-1-yl)methyl)-5-(4-(methylamino)-7H-pyrrolo[2,3-d]pyrimidin-7-yl) cyclopentane-1,2-diol 445 (14 mg, 32%) as a white solid. ¹H NMR (300 MHZ, DMSO-d₆) δ 8.12 (s, 1H), 7.45-7.33 (m, 1H), 7.24 (d, J=3.5 Hz, 1H), 6.52 (d, J=3.5 Hz, 1H), 4.94-4.66 (m, 3H), 4.29 4.17 (m, 1H), 4.16-3.90 (m, 1H), 3.89-3.64 (m, 3H), 3.08-3.00 (m, 2H), 2.95 (d, J=4.6 Hz, 3H), 2.76-2.63 (m, 1H), 2.26-2.13 (m, 3H), 1.95 (s, 1H), 1.56-1.41 (m, 1H). LCMS [conditions: Atlantis T3, 100*4.6 mm, 3 μm, Mobile Phase A: Water/0.05% TFA, Mobile Phase B: Acetonitrile, 0%-20%-95% B-7 min, 1.50 mL/min, ES, m/z]: T_{R}=2.74 min; [M+H]⁺: 359. Chiral-SFC [Column: SB 100×4.6 mm 3.0 μm, Solvent B: MeOH (20 mM NH₃), Oven Temperature: 35° C., Total Flow: 3.00 mL/min: Gradient 50% solvent B for 4 mins]: T_{R}=1.92 min, single peak.

### Synthesis of (1S,2R,3R,5R)-3-{1,6-Diazaspiro[3.4]octan-1-ylmethyl}-5-[4-(methylamino) pyrrolo[2,3-d]pyrimidin-7-yl]cyclopentane-1,2-diol (446)

### tert-Butyl 1-{[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}-1,6-diazaspiro[3.4]octane-6-carboxylate (181a)

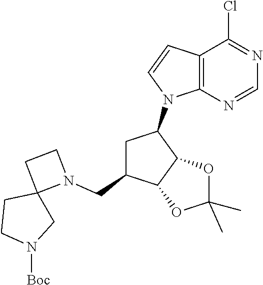 [SMILES: CC(C)(C)OC(=O)N1CCC2(CCN2C[C@H]2C[C@@H](n3ccc4c(Cl)ncnc43)[C@@H]3OC(C)(C)O[C@H]23)C1]

To a stirred solution of (3αR,4S,6R,6αS)-6-{4-chloropyrrolo[2,3-d]pyrimidin-7-yl}-2,2-dimethyl-tetrahydro-3αH-cyclopenta[d][1,3]dioxole-4-carbaldehyde 77a (0.10 g, 0.30 mmol, 1.0 eq) and tert-butyl-1,6-diazaspiro[3.4]octane-6-carboxylate (0.07 g, 0.30 mmol, 1.0 eq) in DCM (1.0 mL) was added NaBH (AcO)₃ (0.10 g, 0.50 mmol, 1.5 eq) at room temperature and the reaction mixture was stirred for 1 h at room temperature. The resulting mixture was basified to pH 8-9 with 10% NaHCO₃ solution and extracted with AcOEt. The organic layer was washed with brine, dried over anhydrous Na₂SO₄ and filtered. The filtrate was concentrated under reduced pressure and the crude residue was purified by silica gel column chromatography (50:50 Petroleum ether: AcOEt) to afford tert-butyl-1-{[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}-1,6-diazaspiro[3.4]octane-6-carboxylate 181a (90 mg, 56%) as a yellow solid. LCMS [conditions Xbridge Shield C18, 50*3.0 mm, 3.5 μm, Mobile Phase A: Water/0.05% Ammonia, Mobile Phase B: Acetonitrile, 5%-100% B-2 min (+), 1.50 L/min, ES, m/z]: T_{R}=1.46 min; [M+H]⁺: 518.

### tert-Butyl-1-{[(3αR,4R,6R,6αS)-2,2-dimethyl-6-[4-(methylamino) pyrrolo[2,3-d]pyrimidin-7-yl]-tetrahydro-3αH-cyclopenta[d][1,3]dioxol-4-yl]methyl}-1,6-diazaspiro[3.4]octane-6-carboxylate (182a)

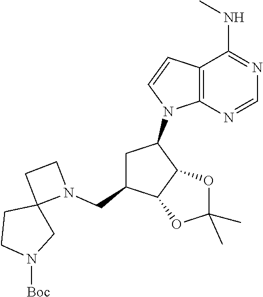 [SMILES: CNc1ncnc2c1ccn2[C@@H]1C[C@H](CN2CCC23CCN(C(=O)OC(C)(C)C)C3)[C@H]2OC(C)(C)O[C@H]21]

To tert-butyl-1-{[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}-1,6-diazaspiro[3.4]octane-6-carboxylate 181a (0.07 g, 0.13 mmol, 1.0 eq) was added methylamine (30% wt. in EtOH, 2.0 mL) at room temperature and the reaction mixture was stirred at 50° C. for 3 h. The resulting mixture was concentrated under reduced pressure to afford tert-butyl-1-{[(3αR,4R,6R,6αS)-2,2-dimethyl-6-[4-(methylamino) pyrrolo[2,3-d]pyrimidin-7-yl]-tetrahydro-3αH-cyclopenta[d][1,3]dioxol-4-yl]methyl}-1,6-diazaspiro[3.4]octane-6-carboxylate 182a (50 mg, crude) as light yellow solid. The crude product was used in the next step directly without further purification. LCMS [conditions Xbridge Shield C18, 50*3.0 mm, 3.5 μm, Mobile Phase A: Water/0.05% Ammonia, Mobile Phase B: Acetonitrile, 5%-100% B-2 min (+), 1.50 L/min, ES, m/z]: T_{R}=1.23 min; [M+H]⁺: 513

### (1S,2R,3R,5R)-3-{1,6-Diazaspiro[3.4]octan-1-ylmethyl}-5-[4-(methylamino) pyrrolo[2,3-d]pyrimidin-7-yl]cyclopentane-1,2-diol (446)

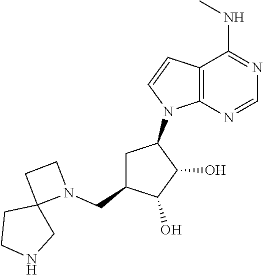 [SMILES: CNc1ncnc2c1ccn2[C@@H]1C[C@H](CN2CCC23CCNC3)[C@@H](O)[C@H]1O]

To a solution of tert-butyl-1-{[(3αR,4R,6R,6αS)-2,2-dimethyl-6-[4-(methylamino) pyrrolo[2,3-d]pyrimidin-7-yl]-tetrahydro-3αH-cyclopenta[d][1,3]dioxol-4-yl]methyl}-1,6-diazaspiro[3.4]octane-6-carboxylate 182a (0.05 g, 0.10 mmol, 1.0 eq) in DCM (1.0 mL) was added TFA (1.0 mL) and the reaction mixture was stirred for 16 h at room temperature. The resulting mixture was concentrated under reduced pressure and the crude residue was dissolved in water (2.0 mL). The solution was adjusted to pH 9 with NH₃.H₂O and the crude product was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 20% B to 43% B in 7 min, 42% B; Wave Length: 220 nm]: to afford (1S,2R,3R,5R)-3-{1,6-diazaspiro[3.4]octan-1-ylmethyl}-5-[4-(methylamino) pyrrolo[2,3-d]pyrimidin-7-yl]cyclopentane-1,2-diol 446 (27 mg, 75%) as an off-white solid. ¹H NMR (400 MHZ, DMSO-d₆) δ8.11 (s, 1H), 7.39-7.31 (m, 1H), 7.26-7.19 (m, 1H), 6.52 (d, J=3.5 Hz, 1H), 4.88-4.59 (m, 2H), 4.22-4.12 (m, 1H), 3.76-3.69 (m, 1H), 3.25-3.10 (m, 2H), 3.10-2.99 (m, 1H), 2.98-2.93 (m, 3H), 2.91-2.75 (m, 1H), 2.74-2.63 (m, 1H), 2.62-2.54 (m, 1H), 2.46-2.30 (m, 2H), 2.22-2.10 (m, 1H), 2.07-1.77 (m, 5H), 1.53-1.37 (m, 1H). LCMS [conditions ZORBAX SB-Aq, 50*4.6 mm, 1.8 μm, Mobile Phase A: Water/0.05% TFA, Mobile Phase B: Acetonitrile, 2%-2%-95% B-3 min, 1.50 L/min, ES, m/z]: T_{R}=0.79; 0.96 min; [M+H]⁺: 373. Chiral-SFC [Column: Lux-4 100×4.6 mm 3.0 μm, Solvent B: MeOH (20 mM NH₃), Oven Temperature: 35° C., Total Flow: 3.00 mL/min, keep Gradient 50% solvent B for 4 mins: two diastereomers]: T_{R}=1.82 min (major peak), T_{R}=2.13 (minor peak); dr=55:45.

### Synthesis of (1S,2R,3R,5R)-3-{1,6-Diazaspiro[3.5]nonan-1-ylmethyl}-5-[4-(methylamino) pyrrolo[2,3-d]pyrimidin-7-yl]cyclopentane-1,2-diol (447)

### Synthesis of tert-butyl-1-{[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}-1,6-diazaspiro[3.5]nonane-6-carboxylate (183a)

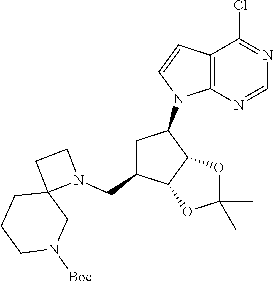 [SMILES: CC(C)(C)OC(=O)N1CCCC2(CCN2C[C@H]2C[C@@H](n3ccc4c(Cl)ncnc43)[C@@H]3OC(C)(C)O[C@H]23)C1]

To a stirred solution of (3αR,4S,6R,6αS)-6-{4-chloropyrrolo[2,3-d]pyrimidin-7-yl}-2,2-dimethyl-tetrahydro-3αH-cyclopenta[d][1,3]dioxole-4-carbaldehyde 77a (0.10 mg, 0.30 mmol, 1.0 eq) and bis(tert-butyl-1,6-diazaspiro[3.5]nonane-6-carboxylate oxalic acid (0.08 g, 0.20 mmol, 0.5 eq) in DCM (1.0 mL) were added TEA (0.03 g, 0.30 mmol, 1.0 eq) and NaBH (AcO)₃ (0.10 g, 0.50 mmol, 1.5 eq) at room temperature and the reaction mixture was stirred for 2 h at room temperature. The resulting mixture was basified to pH 8-9 with 10% NaHCO₃ solution and extracted with AcOEt. The organic layer was washed with brine, dried over anhydrous Na₂SO₄ and filtered. The filtrate was concentrated under reduced pressure and the crude residue was purified by silica gel column chromatography (50:50 Petroleum ether: AcOEt) to afford tert-butyl-1-{[(3ªR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}-1,6-diazaspiro[3.5]nonane-6-carboxylate 183a (90 mg, 54%) as a yellow solid. LCMS [conditions Xbridge Shield C18, 50*3.0 mm, 3.5 μm, Mobile Phase A: Water/0.05% Ammonia, Mobile Phase B: Acetonitrile, 5%-100% B-2 min (+), 1.50 L/min, ES, m/z]: T_{R}=1.46 min; [M+H]⁺: 532.

### tert-Butyl-1-{[(3αR,4R,6R,6αS)-2,2-dimethyl-6-[4-(methylamino) pyrrolo[2,3-d]pyrimidin-7-yl]-tetrahydro-3αH-cyclopenta[d][1,3]dioxol-4-yl]methyl}-1,6-diazaspiro[3.5]nonane-6-carboxylate (184a)

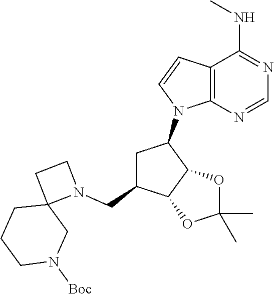 [SMILES: CNc1ncnc2c1ccn2[C@@H]1C[C@H](CN2CCC23CCCN(C(=O)OC(C)(C)C)C3)[C@H]2OC(C)(C)O[C@H]21]

To tert-butyl-1-{[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}-1,6-diazaspiro[3.5]nonane-6-carboxylate 183a (0.08 g, 0.15 mmol, 1.0 eq) was added methylamine (30% wt. in EtOH, 2.0 mL) at room temperature and the reaction mixture was stirred for 3 h at 50° C. The resulting mixture was concentrated under reduced pressure to afford tert-butyl-1-{[(3αR,4R,6R,6αS)-2,2-dimethyl-6-[4-(methylamino) pyrrolo[2,3-d]pyrimidin-7-yl]-tetrahydro-3αH-cyclopenta[d][1,3]dioxol-4-yl]methyl}-1,6-diazaspiro[3.5]nonane-6-carboxylate 184a (0.07 g, crude) as light yellow solid. The crude product was used in the next step without further purification. LCMS [conditions Xbridge

Shield C18, 50*3.0 mm, 3.5 μm, Mobile Phase A: Water/0.05% Ammonia, Mobile Phase B: Acetonitrile, 5%-100% B-2 min (+), 1.50 L/min, ES, m/z]: T_{R}=1.25 min; [M+H]⁺: 527.

### (1S,2R,3R,5R)-3-{1,6-Diazaspiro[3.5]nonan-1-ylmethyl}-5-[4-(methylamino) pyrrolo[2,3-d]pyrimidin-7-yl]cyclopentane-1,2-diol (447)

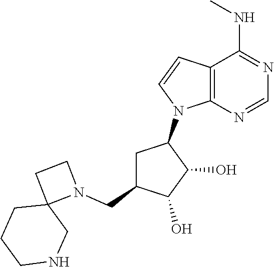 [SMILES: CNc1ncnc2c1ccn2[C@@H]1C[C@H](CN2CCC23CCCNC3)[C@@H](O)[C@H]1O]

To a solution of tert-butyl-1-{[(3αR,4R,6R,6αS)-2,2-dimethyl-6-[4-(methylamino) pyrrolo[2,3-d]pyrimidin-7-yl]-tetrahydro-3αH-cyclopenta[d][1,3]dioxol-4-yl]methyl}-1,6-diazaspiro[3.5]nonane-6-carboxylate 184a (0.07 g, 0.20 mmol, 1.0 eq) in DCM (1.0 mL) was added TFA (1.0 mL) and the reaction mixture was stirred for 16 h at room temperature. The resulting mixture was concentrated under reduced pressure and the crude residue was dissolved in water (2.0 mL). The solution was adjusted to pH 9 with NH₃.H₂O and the crude sample was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 20% B to 51% B in 7 min, 51% B; Wave Length: 220 nm]: to afford (1S,2R,3R,5R)-3-{1,6-diazaspiro[3.5]nonan-1-ylmethyl}-5-[4-(methylamino) pyrrolo[2,3-d]pyrimidin-7-yl]cyclopentane-1,2-diol 447 (34.3 mg, 67%) as an off-white solid. ¹H NMR (400 MHZ, DMSO-d₆) δ8.11 (s, 1H), 7.40-7.32 (m, 1H), 7.22 (d, J=3.5 Hz, 1H), 6.52 (d, J=3.5 Hz, 1H), 4.85-4.76 (m, 1H), 4.75-4.66 (m, 1H), 4.24-4.11 (m, 1H), 3.75-3.67 (m, 1H), 3.21-3.00 (m, 2H), 2.95 (d, J=4.7 Hz, 3H), 2.86-2.76 (m, 1H), 2.75-2.68 (m, 1H), 2.68-2.60 (m, 1H), 2.48-2.38 (m, 1H), 2.35-2.23 (m, 1H), 2.22-2.09 (m, 1H), 1.93-1.69 (m, 4H), 1.62-1.40 (m, 3H), 1.40-1.24 (m, 1H). LCMS [conditions ZORBAX SB-Aq, 50*4.6 mm, 1.8 μm, Mobile Phase A: Water/0.05% TFA, Mobile Phase B: Acetonitrile, 2%-2%-95% B-3 min-1.5.lcm, 1.50 L/min, ES, m/z]: T_{R}=1.03; 1.16 min; [M+H]⁺: 387. Chiral-SFC [Column: Lux-4 100×4.6 mm 3.0 μm, Solvent B: MeOH (20 mM NH₃), Oven Temperature: 35° C., Total Flow: 3.00 mL/min. keep Gradient 50% solvent B for 4 mins: two diastereomers]: T_{R}=1.94 min (major peak), T_{R}=2.29 (minor peak); dr=51.7:48.3.

### Synthesis of (1S,2R,3R,5R)-3-{1,6-diazaspiro[3.5]nonan-1-ylmethyl}-5-[4-(methylamino) pyrrolo[2,3-d]pyrimidin-7-yl]cyclopentane-1,2-diol (448)

### tert-Butyl-6-{[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}-2,6-diazaspiro[4.5]decane-2-carboxylate (185a)

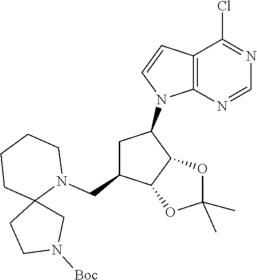 [SMILES: CC(C)(C)OC(=O)N1CCC2(CCCCN2C[C@H]2C[C@@H](n3ccc4c(Cl)ncnc43)[C@@H]3OC(C)(C)O[C@H]23)C1]

To a stirred solution of (3αR,4S,6R,6αS)-6-{4-chloropyrrolo[2,3-d]pyrimidin-7-yl}-2,2-dimethyl-tetrahydro-3αH-cyclopenta[d][1,3]dioxole-4-carbaldehyde 77a (0.10 g, 0.30 mmol, 1.0 eq) and tert-butyl-2,6-diazaspiro[4.5]decane-2-carboxylate (0.07 g, 0.30 mmol, 1.0 eq) in DCM (1.0 mL) was added NaBH (AcO)₃ (0.10 g, 0.50 mmol, 1.5 eq) and the reaction mixture was stirred for 1 h at room temperature. The resulting mixture was basified to pH 8-9 with 10% NaHCO₃ solution. and extracted with AcOEt. The organic layer was washed with brine, dried over anhydrous Na₂SO₄ and filtered. The filtrate was concentrated under reduced pressure and the crude residue was purified by silica gel column chromatography (50:50 Petroleum ether: AcOEt) to afford tert-butyl-6-{[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}-2,6-diazaspiro[4.5]decane-2-carboxylate 185a (0.10 g, 59%) as a yellow solid. LCMS [conditions Xbridge Shield C18, 50*3.0 mm, 3.5 μm, Mobile Phase A: Water/0.05% Ammonia, Mobile Phase B: Acetonitrile, 5%-100% B-2 min (+), 1.50 L/min, ES, m/z]: T_{R}=1.54 min; [M+H]⁺: 546.

### tert-Butyl-6-{[(3αR,4R,6R,6αS)-2,2-Dimethyl-6-[4-(methylamino) pyrrolo[2,3-d]pyrimidin-7-yl]-tetrahydro-3αH-cyclopenta[d][1,3]dioxol-4-yl]methyl}-2,6-diazaspiro[4.5]decane-2-carboxylate (186a)

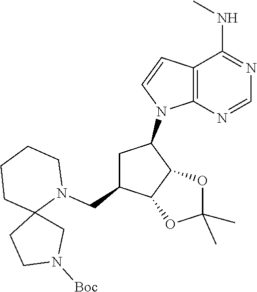 [SMILES: CNc1ncnc2c1ccn2[C@@H]1C[C@H](CN2CCCCC23CCN(C(=O)OC(C)(C)C)C3)[C@H]2OC(C)(C)O[C@H]21]

To tert-butyl-6-{[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}-2,6-diazaspiro[4.5]decane-2-carboxylate 185a (0.08 g, 0.20 mmol, 1.0 eq) and methylamine (30% wt. in ethanol, 2.0 mL) at room temperature and the reaction mixture was stirred at 50° C. for 3 h. The resulting mixture was concentrated under reduced pressure to afford tert-butyl-6-{[(3αR,4R,6R,6αS)-2,2-dimethyl-6-[4-(methylamino) pyrrolo[2,3-d]pyrimidin-7-yl]-tetrahydro-3αH-cyclopenta[d][1,3]dioxol-4-yl]methyl}-2,6-diazaspiro[4.5]decane-2-carboxylate 186a (60 mg, crude) as a yellow solid. The crude product was used in the next step directly without further purification. LCMS [conditions Xbridge Shield C18, 50*3.0 mm, 3.5 μm, Mobile Phase A: Water/0.05% Ammonia, Mobile Phase B: Acetonitrile, 5%-100% B-2 min (+), 1.50 L/min, ES, m/z]: T_{R}=1.54 min; [M+H]⁺: 541

### (1S,2R,3R,5R)-3-{1,6-Diazaspiro[3.5]nonan-1-ylmethyl}-5-[4-(methylamino) pyrrolo[2,3-d]pyrimidin-7-yl]cyclopentane-1,2-diol (448)

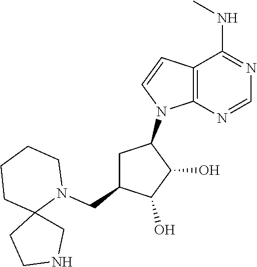 [SMILES: CNc1ncnc2c1ccn2[C@@H]1C[C@H](CN2CCCCC23CCNC3)[C@@H](O)[C@H]1O]

To tert-butyl-6-{[(3αR,4R,6R,6αS)-2,2-dimethyl-6-[4-(methylamino) pyrrolo[2,3-d]pyrimidin-7-yl]-tetrahydro-3αH-cyclopenta[d][1,3]dioxol-4-yl]methyl}-2,6-diazaspiro[4.5]decane-2-carboxylate 186a (60 mg, 0.10 mmol, 1.0 eq) in DCM (1.0 mL) was added TFA (1.0 mL) and the reaction mixture was stirred for 16 h at room temperature. The resulting mixture was concentrated under reduced pressure, dissolved in water (2.0 mL) and the solution was adjusted to pH 9 with NH₃.H₂O. The crude residue was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 22% B to 52% B in 7 min, 52% B; Wave Length: 220 nm]: to afford (1S,2R,3R,5R)-3-{2,6-diazaspiro[4.5]decan-6-ylmethyl}-5-[4-(methylamino) pyrrolo[2,3-d]pyrimidin-7-yl]cyclopentane-1,2-diol 448 (37.7 mg, 85%) as an off-white-solid. ¹H NMR (400 MHz, DMSO-d₆) δ8.15-8.08 (m, 1H), 7.40-7.32 (m, 1H), 7.26-7.19 (m, 1H), 6.55-6.49 (m, 1H), 4.88-4.68 (m, 2H), 4.29-4.19 (m, 1H), 3.84-3.71 (m, 1H), 3.31-3.10 (m, 2H), 3.00-2.91 (m, 3H), 2.90-2.79 (m, 1H), 2.77-2.64 (m, 1H), 2.62-2.53 (m, 1H), 2.38-2.26 (m, 1H), 2.24-2.09 (m, 1H), 2.08-1.93 (m, 1H), 1.78-1.65 (m, 1H), 1.54-1.33 (m, 7H). LCMS [conditions ZORBAX SB-Aq, 50*4.6 mm, 1.8 μm, Mobile Phase A: Water/0.05% TFA, Mobile Phase B: Acetonitrile, 5%-50%-95% B-5 min, 1.50 L/min, ES, m/z]: T_{R}=0.84; 0.89 min; [M+H]⁺: 401. Chiral-SFC [Column: Lux-4 100×4.6 mm 3.0 μm, Solvent B: MeOH (20 mM NH₃), Oven Temperature: 35° C., Total Flow: 3.00 mL/min. keep Gradient 50% solvent B for 4 mins]: 2 diastereomers; T_{R}=2.17 min (major peak), T_{R}=2.51 (minor peak); dr=53.5:46.5.

### Synthesis of (1S,2R,3R,5R)-3-(((S)-1,7-Diazaspiro[4.5]decan-1-yl)methyl)-5-(4-(methylamino)-7H-pyrrolo[2,3-d]pyrimidin-7-yl) cyclopentane-1,2-diol (449) and (1S,2R,3R,5R)-3-(((R)-1,7-diazaspiro[4.5]decan-1-yl)methyl)-5-(4-(methylamino)-7H-pyrrolo[2,3-d]pyrimidin-7-yl) cyclopentane-1,2-diol (450)

### tert-Butyl-1-(((3αR,4R,6R,6αS)-6-(4-chloro-7H-pyrrolo[2,3-d]pyrimidin-7-yl)-2,2-dimethyltetrahydro-4H-cyclopenta[d][1,3]dioxol-4-yl)methyl)-1,7-diazaspiro[4.5]decane-7-carboxylate (187a)

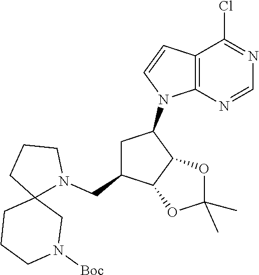 [SMILES: CC(C)(C)OC(=O)N1CCCC2(CCCN2C[C@H]2C[C@@H](n3ccc4c(Cl)ncnc43)[C@@H]3OC(C)(C)O[C@H]23)C1]

To (3αR,4S,6R,6αS)-6-{4-chloropyrrolo[2,3-d]pyrimidin-7-yl}-2,2-dimethyl-tetrahydro-3αH-cyclopenta[d][1,3]dioxole-4-carbaldehyde 77 (90 mg, 0.30 mmol, 1.0 eq), tert-butyl-1,7-diazaspiro[4.5]decane-7-carboxylate (80 mg, 0.40 mmol, 1.2 eq) in DCM (2.0 mL) was added NaBH (OAc)₃ (118 mg, 0.60 mmol, 2 eq) and AcOH (168 mg, 2.80 mmol, 10 eq) and the resulting mixture was stirred for 1 h at room temperature. The resulting mixture was basified to pH 8-9 with 10% NaHCO₃ solution. and extracted with AcOEt. The combined organic layers were washed with brine, dried over anhydrous Na₂SO₄ and filtered. The filtrate was concentrated under reduced pressure and the crude residue was purified by silica gel column chromatography (50:50 Petroleum ether: AcOEt) to afford tert-butyl-1-{[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}-1,7-diazaspiro[4.5]decane-7-carboxylate 187a (46 mg, 30%) as a yellow solid. LCMS [XBridge C18, 50*3.0 mm, 3.5 μm; Mobile Phase A: Water/5 mM NH₄HCO₃, Mobile Phase B: MeOH, 10%-95% B-3 min (+), 1.50 mL/min, ES, m/z]: T_{R}=2.36 min; [M+H]⁺: 546.

### tert-Butyl-1-(((3αR,4R,6R,6αS)-2,2-dimethyl-6-(4-(methylamino)-7H-pyrrolo[2,3-d]pyrimidin-7-yl) tetrahydro-4H-cyclopenta[d][1,3]dioxol-4-yl)methyl)-1,7-diazaspiro[4.5]decane-7-carboxylate (188a)

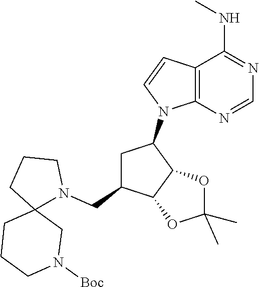 [SMILES: CNc1ncnc2c1ccn2[C@@H]1C[C@H](CN2CCCC23CCCN(C(=O)OC(C)(C)C)C3)[C@H]2OC(C)(C)O[C@H]21]

To tert-butyl-1-{[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}-1,7-diazaspiro[4.5]decane-7-carboxylate 187a (46 mg, 0.08 mmol, 1.0 eq) was added methylamine (30% wt. in EtOH, 2.0 mL) at room temperature and the reaction mixture was stirred at 50° C. for 3 h. The resulting mixture was concentrated under reduced pressure to afford tert-butyl-1-{[(3αR,4R,6R,6αS)-2,2-dimethyl-6-[4-(methylamino) pyrrolo[2,3-d]pyrimidin-7-yl]-tetrahydro-3αH-cyclopenta[d][1,3]dioxol-4-yl]methyl}-1,7-diazaspiro[4.5]decane-7-carboxylate 188a (35 mg, 77%) as a yellow oil. LCMS [XBridge C18, 50*3.0 mm, 3.5 μm; Mobile Phase A: Water/5 mM NH₄HCO₃, Mobile Phase B: MeOH, 10%-95% B-2 min (+), 1.50 mL/min, ES, m/z]: T_{R}=1.53 min; [M+H]⁺: 541.

### (1S,2R,3R,5R)-3-(((S)-1,7-diazaspiro[4.5]decan-1-yl)methyl)-5-(4-(methylamino)-7H-pyrrolo[2,3-d]pyrimidin-7-yl) cyclopentane-1,2-diol (449) & (1S,2R,3R,5R)-3-(((R)-1,7-diazaspiro[4.5]decan-1-yl)methyl)-5-(4-(methylamino)-7H-pyrrolo[2,3-d]pyrimidin-7-yl) cyclopentane-1,2-diol (450)

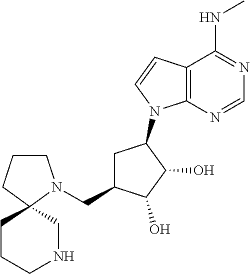 [SMILES: CNc1ncnc2c1ccn2[C@@H]1C[C@H](CN2CCC[C@]23CCCNC3)[C@@H](O)[C@H]1O]

To tert-butyl-1-{[(3αR,4R,6R,6αS)-2,2-dimethyl-6-[4-(methylamino) pyrrolo[2,3-d]pyrimidin-7-yl]-tetrahydro-3αH-cyclopenta[d][1,3]dioxol-4-yl]methyl}-1,7-diazaspiro[4.5]decane-7-carboxylate 188a (35 mg, 0.06 mmol, 1.0 eq) in DCM (0.30 mL) was added TFA (0.30 mL) and the reaction mixture was stirred for 16 h at room temperature. The resulting mixture was concentrated under reduced pressure and the mixture was basified to pH 10 with NH₃.H₂O. The crude residue was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 22% B to 58% B in 7 min, 58% B; Wave Length: 220 nm]: to afford (1S,2R,3R,5R)-3-[(5S)-1,7-diazaspiro[4.5]decan-1-ylmethyl]-5-[4-(methylamino) pyrrolo[2,3-d]pyrimidin-7-yl]cyclopentane-1,2-diol or (1S,2R,3R,5R)-3-[(5R)-1,7-diazaspiro[4.5]decan-1-ylmethyl]-5-[4-(methylamino) pyrrolo[2,3-d]pyrimidin-7-yl]cyclopentane-1,2-diol 449 (5.1 mg, 19%, 1st eluting isomer) as an off-white solid. ¹H NMR (400 MHZ, Chloroform-d) 88.37 (s, 1H), 7.01 (d, J=3.7 Hz, 1H), 6.39 (d, J=3.7 Hz, 1H), 5.08-4.97 (m, 2H), 4.49-4.41 (m, 1H), 4.38-4.28 (m, 1H), 3.27-3.11 (m, 5H), 3.07-2.96 (m, 1H), 2.83-2.68 (m, 3H), 2.66-2.55 (m, 3H), 2.29-2.19 (m, 1H), 2.18-2.01 (m, 2H), 1.94-1.72 (m, 5H), 1.71-1.39 (m, 4H). LCMS [conditions BEH Phenyl, 100*3.0 mm, 2.5 μm, Mobile Phase A: Water/0.05% ammonia water, Mobile Phase B: Acetonitrile, 5%-95% B-8.0 min (+), 0.80 mL/min, ES, m/z]: T_{R}=3.01 min; [M+H]⁺: 401. UPLC [conditions: Column name: BEH Phenyl, 100*3.0 mm, 2.5 μm, Mobile Phase A: Water/0.05% ammonia water, Mobile Phase B: Acetonitrile, 5%-95% B-8.0 min (+), 0.80 mL/min: two diastereomersl. T. =2 97 min. T. =3.04 min: dr=25:1

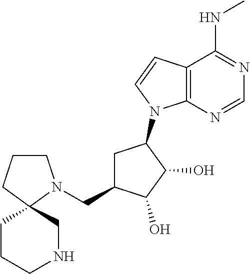 [SMILES: CNc1ncnc2c1ccn2[C@@H]1C[C@H](CN2CCC[C@@]23CCCNC3)[C@@H](O)[C@H]1O]

Prep-HPLC as above also afforded (1S,2R,3R,5R)-3-[(5R)-1,7-diazaspiro[4.5]decan-1-ylmethyl]-5-[4-(methylamino) pyrrolo[2,3-d]pyrimidin-7-yl]cyclopentane-1,2-diol 450 or (1S,2R,3R,5R)-3-[(5S)-1,7-diazaspiro[4.5]decan-1-ylmethyl]-5-[4-(methylamino) pyrrolo[2,3-d]pyrimidin-7-yl]cyclopentane-1,2-diol (5.0 mg, 19%, 2nd eluting isomer) as an off-white solid. ¹H NMR (400 MHz, Chloroform-d) δ 8.37 (s, 1H), 7.01 (d, J=3.6 Hz, 1H), 6.39 (d, J=3.6 Hz, 1H), 5.15-4.93 (m, 2H), 4.51-4.38 (m, 1H), 4.33-4.24 (m, 1H), 3.27-3.10 (m, 5H), 3.02-2.94 (m, 1H), 2.87-2.80 (m, 1H), 2.76-2.48 (m, 5H), 2.29-2.19 (m, 1H), 2.18-2.09 (m, 1H), 1.93-1.79 (m, 4H), 1.79-1.45 (m, 6H). LCMS [conditions BEH Phenyl, 100*3.0 mm, 2.5 μm, Mobile Phase A: Water/0.05% ammonia water, Mobile Phase B: Acetonitrile, 5%-95% B-8.0 min (+), 0.80 mL/min, ES, m/z]: T_{R}=3.26 min; [M+H]*: 401. UPLC conditions [Column name: BEH Phenyl, 100*3.0 mm, 2.5 μm, Mobile Phase A: Water/0.05% ammonia water, Mobile Phase B: Acetonitrile, 5%-95% B-8.0 min (+), 0.80 mL/min]: T_{R}=5.16 min (major peak, KMI1020751b or KMI1020751a), T_{R}=4.99 min; dr=18:1.

### Synthesis of (1S,2R,3R,5R)-3-((1,8-diazaspiro[4.5]decan-1-yl)methyl)-5-(4-(methylamino)-7H-pyrrolo[2,3-d]pyrimidin-7-yl) cyclopentane-1,2-diol (451)

### tert-Butyl-1-(((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl)-1,8-diazaspiro[4.5]decane-8-carboxylate (189a)

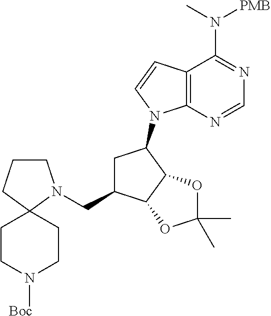

To a solution of (3αR,4S,6R,6αS)-6-(4-{[(4-methoxyphenyl)methyl](methyl) amino}pyrrolo[2,3-d]pyrimidin-7-yl)-2,2-dimethyl-tetrahydro-3αH-cyclopenta[d][1,3]dioxole-4-carbaldehyde 19a (70 mg, 0.16 mmol, 1.0 eq) and tert-butyl-1,8-diazaspiro[4.5]decane-8-carboxylate (38.5 mg, 0.16 mmol, 1.0 eq) in DCM (1.0 mL) were added sodium triacetoxyborohydride (51 mq, 0.24 mmol, 1.5 eq) and AcOH (96 mq, 1.6 mmol, 10 eq) at room temperature and the reaction mixture was stirred for 1 h at room temperature. The resulting mixture was basified to pH 8-9 with 10% NaHCO₃ solution. and extracted with AcOEt. The combined organic layers were washed with brine, dried over anhydrous Na₂SO₄ and filtered. The filtrate was concentrated under reduced pressure and the crude residue was purified by silica gel column chromatography (40:60 Petroleum ether: AcOEt) to afford tert-butyl-1-(((3αR,4R,6R,6αS)-6-(4-((4-methoxybenzyl)(methyl) amino)-7H-pyrrolo[2,3-d]pyrimidin-7-yl)-2,2-dimethyltetrahydro-4H-cyclopenta[d][1,3]dioxol-4-yl)methyl)-1,8-diazaspiro[4.5]decane-8-carboxylate 189a (70 mg, 66%) as an off-white semi-solid. LCMS [XBridge C18, 50*3.0 mm, 3.5 μm; Mobile Phase A: Water/5 mM NH₄HCO₃, Mobile Phase B: MeOH, 10%-95% B-3 min (+), 1.50 mL/min, ES, m/z]: T_{R}=2.22 min; [M+H]⁺: 661.

### (1S,2R,3R,5R)-3-((1,8-Diazaspiro[4.5]decan-1-yl)methyl)-5-(4-(methylamino)-7H-pyrrolo[2,3-d]pyrimidin-7-yl) cyclopentane-1,2-diol (451)

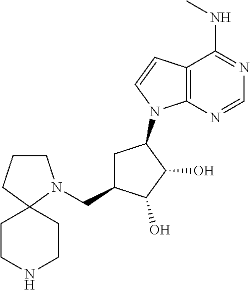 [SMILES: CNc1ncnc2c1ccn2[C@@H]1C[C@H](CN2CCCC23CCNCC3)[C@@H](O)[C@H]1O]

To tert-butyl-1-{[(3αR,4R,6R,6αS)-6-(4-{[(4-methoxyphenyl)methyl](methyl) amino}pyrrolo[2,3-d]pyrimidin-7-yl)-2,2-dimethyl-tetrahydro-3αH-cyclopenta[d][1,3]dioxol-4-yl]methyl}-1,8-diazaspiro[4.5]decane-8-carboxylate 189a (50 mg, 0.076 mmol, 1.0 eq) was added TFA (1.0 mL) and the reaction mixture was stirred for 16 h at room temperature. The resulting mixture was concentrated under reduced pressure and the crude product was purified by Prep-HPLC [Column, XBridge Shield RP18 OBD Column, 19*150 mm, 5 μm; mobile phase, ACN and Water (10 mmol/L NH₄HCO₃+0.1% NH₃.H₂O)(18% up to 43% in 8 min); Detector, UV 220 nm]: to afford (1S,2R,3R,5R)-3-{1,8-diazaspiro[4.5]decan-1-ylmethyl}-5-[4-(methylamino) pyrrolo[2,3-d]pyrimidin-7-yl]cyclopentane-1,2-diol 451 (16 mg, 52%) as a white solid. ¹H NMR (400 MHZ, DMSO-d₆) δ8.10 (s, 1H), 7.22 (d, J=3.7 Hz, 1H), 6.52 (d, J=3.6 Hz, 1H), 4.89-4.75 (m, 1H), 4.31-4.20 (m, 1H), 3.97 (s, 1H), 3.80-3.68 (m, 1H), 3.11-2.82 (m, 4H), 2.81-2.68 (m, 2H), 2.65-2.56 (m, 4H), 2.43-2.29 (m, 1H), 2.27-2.13 (m, 1H), 2.09-1.96 (m, 1H), 1.75-1.62 (m, 4H), 1.60-1.38 (m, 4H), 1.26-1.07 (m, 2H). LCMS [conditions: HPH-C18, 50*3.0 mm, 2.7 μm, Mobile Phase A: Water/0.05% ammonia water, Mobile Phase B: Acetonitrile, 5%-40%-95% B-5.0 min (+), 1.20 mL/min, ES, m/z]: T_{R}=1.92 min; [M+H]⁺: 401. Chiral-SFC [Column: IG 100×4.6 mm 3.0 μm, Solvent B: MeOH (20 mM NH₃), Oven Temperature: 35° C., Total Flow: 3.00 mL/min. Gradient 50% solvent B for 4 mins]: T_{R}=2.47 min, single peak.

### Synthesis of (1S,2R,35,4R)-4-(4-amino-5-bromo-7H-pyrrolo[2,3-d]pyrimidin-7-yl)-2,3-dihydroxy-N-(3-(phenethylamino)propyl) cyclopentane-1-carboxamide (452)

### Methyl(1S,2R,3S,4R)-4-(4-chloro-7H-pyrrolo[2,3-d]pyrimidin-7-yl)-2,3-dihydroxycyclopentane-1-carboxylate (21a)

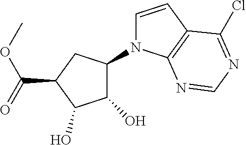 [SMILES: COC(=O)[C@H]1C[C@@H](n2ccc3c(Cl)ncnc32)[C@H](O)[C@@H]1O]

To methyl(1S,2R,3S,4R)-4-amino-2,3-dihydroxycyclopentane-1-carboxylate hydrochloride 2 (500 mg, 2.36 mmol, 1.0 eq) and 2-(4,6-dichloropyrimidin-5-yl)acetaldehyde (496 mg, 2.60 mmol, 1.1 eq) in EtOH (5.0 mL) was added TEA (717 mg, 7.08 mmol, 3.0 eq) at room temperature and the reaction mixture was stirred at 80° C. for 3 h. The resulting mixture was allowed to cool down to room temperature and concentrated under vacuum to afford methyl(1S,2R,3S,4R)-4-{4-chloropyrrolo[2,3-d]pyrimidin-7-yl}-2,3-dihydroxycyclopentane-1-carboxylate 21a (600 mg, crude) as a brown solid. The crude product was used to the next step directly without further purification. LCMS [conditions: XBridge C18, 50*3.0 mm, 3.5 μm, Mobile Phase A: Water/5 mM NH₄HCO₃, Mobile Phase B: Methanol, 10%-95% B-2.5 min (+−), 1.50 mL/min, ES, m/z]: T_{R}=1.35 min; [M+H]⁺: 312.

### Methyl(3αR,4S,6R,6αS)-6-(4-chloro-7H-pyrrolo[2,3-d]pyrimidin-7-yl)-2,2-dimethyltetrahydro-4H-cyclopenta[d][1,3]dioxole-4-carboxylate (22a)

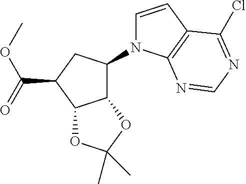 [SMILES: COC(=O)[C@H]1C[C@@H](n2ccc3c(Cl)ncnc32)[C@@H]2OC(C)(C)O[C@@H]21]

To (1S,2R,3S,4R)-4-{4-chloropyrrolo[2,3-d]pyrimidin-7-yl}-2,3-dihydroxycyclopentane-1-carboxylate 21a (600 mg, crude) and 2,2-dimethoxypropane (1.00 g, 9.63 mmol, 5.0 eq) in acetone (10 mL) was added TsOH (663 mg, 3.85 mmol, 2.0 eq) at room temperature and the reaction mixture was stirred for 1 h at this temperature. The resulting mixture was basified to pH>9 with saturated NaHCO₃ solution and then extracted with AcOEt (45 mL). The organic layer was washed with brine (15 mL) and dried over anhydrous Na₂SO₄. After filtration, the filtrate was concentrated under reduced pressure and the crude residue was purified by silica gel column chromatography (2/1 Petrolium ether/AcOEt) to afford methyl(3αR,4S,6R,6αS)-6-{4-chloropyrrolo[2,3-d]pyrimidin-7-yl}-2,2-dimethyl-tetrahydro-3αH-cyclopenta[d][1,3]dioxole-4-carboxylate 22a (500 mg, 74%) as a light yellow solid. LCMS [conditions: Cortecs C_{18+, 50*3.0} mm, 2.7 μm, Mobile Phase A: Water/0.05% TFA, Mobile Phase B: Acetonitrile, 2%-100% B-2.5 min (+)-P, 1.50 mL/min, ES, m/z]: T_{R}=1.43 min; [M+H]⁺: 352, 354.

### Methyl(3αR,4S,6R,6αS)-6-(5-bromo-4-chloro-7H-pyrrolo[2,3-d]pyrimidin-7-yl)-2,2-dimethyltetrahydro-4H-cyclopenta[d][1,3]dioxole-4-carboxylate (23a)

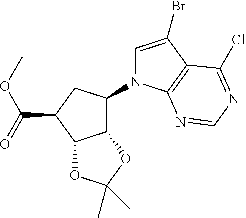 [SMILES: COC(=O)[C@H]1C[C@@H](n2cc(Br)c3c(Cl)ncnc32)[C@@H]2OC(C)(C)O[C@@H]21]

To a stirred solution of methyl(3αR,4S,6R,6αS)-6-{4-chloropyrrolo[2,3-d]pyrimidin-7-yl}-2,2-dimethyl-tetrahydro-3αH-cyclopenta[d][1,3]dioxole-4-carboxylate 22a (500 mg, 1.42 mmol, 1.0 eq) in DMF (5.0 mL), at room temperature, was added NBS (304 mg, 1.71 mmol, 1.2 eq) in several portions and the reaction mixture was stirred for 1 h. The resulting mixture was purified by reverse flash chromatography [column, C18 silica gel; mobile phase, CH₃CN in water, 10% to 50% gradient in 12 min; detector, UV 254 nm]: afforded methyl(3αR,4S,6R,6αS)-6-{5-bromo-4-chloropyrrolo[2,3-d]pyrimidin-7-yl}-2,2-dimethyl-tetrahydro-3αH-cyclopenta[d][1,3]dioxole-4-carboxylate 23a (460 mg, 75%) as an off-white solid. ¹H NMR (400 MHZ, DMSO-d₆) δ 8.69 (s, 1H), 8.25 (s, 1H), 5.27-5.14 (m, 1H), 5.00-4.92 (m, 2H), 3.66 (s, 3H), 3.18-3.06 (m, 1H), 2.60-2.52 (m, 2H), 1.50 (s, 3H), 1.24 (s, 3H).

### (3αR,4S,6R,6αS)-6-(5-Bromo-4-chloro-7H-pyrrolo[2,3-d]pyrimidin-7-yl)-2,2-dimethyltetrahydro-4H-cyclopenta[d][1,3]dioxole-4-carboxylic acid (24a)

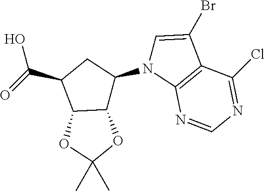 [SMILES: CC1(C)O[C@@H]2[C@H](O1)[C@@H](C(=O)O)C[C@H]2n1cc(Br)c2c(Cl)ncnc21]

Methyl(3αR,4S,6R,6αS)-6-{5-bromo-4-chloropyrrolo[2,3-d]pyrimidin-7-yl}-2,2-dimethyl-tetrahydro-3αH-cyclopenta[d][1,3]dioxole-4-carboxylate 23a (400 mg, 0.93 mmol, 1.0 equiv) was dissolved in THF (4.0 mL) and H₂O (2.0 mL) and then NaOH (74 mg, 1.86 mmol, 2.0 equiv) was added and the reaction mixture was stirred for 2 h at room temperature. The resulting mixture was concentrated under vacuum and purified by reverse flash chromatography with the following conditions [column, C₁₈ silica gel; mobile phase, CH₃CN in water, 10% to 30% gradient in 10 min; detector, UV 254 nm]: to afford (3αR,4S,6R,6αS)-6-{5-bromo-4-chloropyrrolo[2,3-d]pyrimidin-7-yl}-2,2-dimethyl-tetrahydro-3αH-cyclopenta[d][1,3]dioxole-4-carboxylic acid 24a (180 mg, 47%) as a white solid. ¹H NMR (300 MHZ, DMSO-d₆) δ 8.68 (s, 1H), 8.42 (s, 1H), 5.11 (td, J=8.0, 4.3 Hz, 1H), 4.95 (dd, J=6.5, 3.0 Hz, 1H), 4.83-4.63 (m, 1H), 2.67-2.59 (m, 1H), 2.46-2.41 (m, 1H), 2.38-2.21 (m, 1H), 1.46 (s, 3H), 1.22 (s, 3H). Chiral SFC [Volume: Column: Amylose C Neo 100×4.6 mm 3.0 μm; Co Solvent: B: MeOH; Start Conc. of Pump B: 30.0%; Method Filename: 30-50%; Total Flow: 3.00 mL/min; BPR Pressure: 15.00 MPa; BPR Temperature: 50° C.]: T_{R}=1.18 min; single peak.

### tert-Butyl-(3-((3αR,4S,6R,6αS)-6-(5-bromo-4-chloro-7H-pyrrolo[2,3-d]pyrimidin-7-yl)-2,2-dimethyltetrahydro-4H-cyclopenta[d][1,3]dioxole-4-carboxamido)propyl)(phenethyl)carbamate (25a)

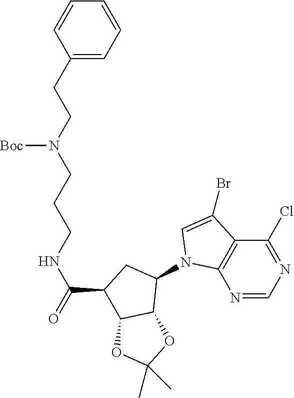 [SMILES: CC(C)(C)OC(=O)N(CCCNC(=O)[C@H]1C[C@@H](n2cc(Br)c3c(Cl)ncnc32)[C@@H]2OC(C)(C)O[C@@H]21)CCc1ccccc1]

(3αR,4S,6R,6αS)-6-{5-bromo-4-chloropyrrolo[2,3-d]pyrimidin-7-yl}-2,2-dimethyl-tetrahydro-3αH-cyclopenta[d][1,3]dioxole-4-carboxylic acid 24a (180 mg, 0.43 mmol, 1.0 eq) and HATU (197 mg, 0.52 mmol, 1.2 eq) were dissolved in DMF (3.0 mL) and DIEA (168 mg, 1.29 mmol, 3.0 eq) was added. The resulting mixture was stirred for 10 min at room temperature and then tert-butyl-N-(3-aminopropyl)-N-(2-phenylethyl)carbamate (144 mg, 0.52 mmol, 1.2 eq) was added and the reaction mixture was stirred for 2 h at room temperature. The resulting mixture was purified by reverse flash chromatography [C₁₈-column, mobile phase, MeCN in water, 20% to 70% gradient in 12 min; detector, UV 220 nm]: to afford tert-butyl-N-(3-{[(3αR,4S,6R,6αS)-6-{5-bromo-4-chloropyrrolo[2,3-d]pyrimidin-7-yl}-2,2-dimethyl-tetrahydro-3αH-cyclopenta[d][1,3]dioxol-4-yl]formamido}propyl)-N-(2-phenylethyl)carbamate 25a (100 mg, 34%) as a light yellow solid. 1H NMR (300 MHZ, Chloroform-d) 8 8.65 (s, 1H), 7.50 (s, 1H), 7.37-7.29 (m, 2H), 7.28-7.03 (m, 4H), 5.23-5.19 (m, 1H), 5.01 (s, 1H), 4.88 (dd, J=7.0, 5.0 Hz, 1H), 3.56-3.06 (m, 6H), 3.00-2.74 (m, 3H), 2.72-2.46 (m, 2H), 1.83 (s, 3H), 1.47 (s, 9H), 1.39 (s, 3H), 1.38-1.21 (m, 2H).

### tert-Butyl-(3-((3αR,4S,6R,6αS)-6-(4-amino-5-bromo-7H-pyrrolo[2,3-d]pyrimidin-7-yl)-2,2-dimethyltetrahydro-4H-cyclopenta[d][1,3]dioxole-4-carboxamido)propyl)(phenethyl)carbamate (26a)

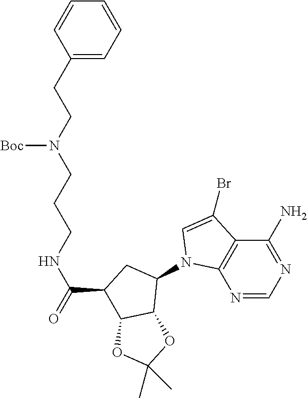 [SMILES: CC(C)(C)OC(=O)N(CCCNC(=O)[C@H]1C[C@@H](n2cc(Br)c3c(N)ncnc32)[C@@H]2OC(C)(C)O[C@@H]21)CCc1ccccc1]

To tert-butyl-N-(3-{[(3αR,4S,6R,6αS)-6-{5-bromo-4-chloropyrrolo[2,3-d]pyrimidin-7-yl}-2,2-dimethyl-tetrahydro-3αH-cyclopenta[d][1,3]dioxol-4-yl]formamido}propyl)-N-(2-phenylethyl)carbamate 25a (100 mg, 0.15 mmol, 1.0 eq) was added in NH₃ (3.0 mL, 7M in MeOH) in a sealed tube and the reaction mixture was stirred at 75° C. for 8 h. The resulting mixture was concentrated under vacuum to afford tert-butyl-(3-((3αR,4S,6R,6αS)-6-(4-amino-5-bromo-7H-pyrrolo[2,3-d]pyrimidin-7-yl)-2,2-dimethyltetrahydro-4H-cyclopenta[d][1,3]dioxole-4-carboxamido)propyl)(phenethyl)carbamate 26a (60 mg, crude) as a light yellow solid. The crude product was used in the next step directly without further purification. LCMS [conditions: Kinetex XB-C18, 3.0*30 mm, Mobile Phase A: Water/0.1% FA, Mobile Phase B: Acetonitrile/0.05% FA, 5%-95% B-2 min (+), 1.20 mL/min, ES, m/z]: T_{R}=1.19 min; [M+H]⁺: 657, 659.

### (1S,2R,3S,4R)-4-(4-Amino-5-bromo-7H-pyrrolo[2,3-d]pyrimidin-7-yl)-2,3-dihydroxy-N-(3-(phenethylamino)propyl) cyclopentane-1-carboxamide (452)

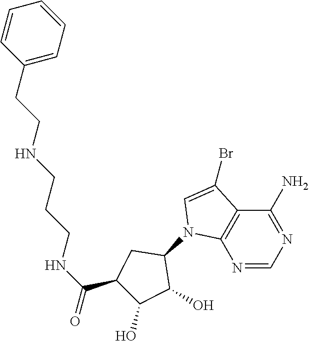 [SMILES: Nc1ncnc2c1c(Br)cn2[C@@H]1C[C@H](C(=O)NCCCNCCc2ccccc2)[C@@H](O)[C@H]1O]

To tert-butyl-(3-((3αR,4S,6R,6αS)-6-(4-amino-5-bromo-7H-pyrrolo[2,3-d]pyrimidin-7-yl)-2,2-dimethyltetrahydro-4H-cyclopenta[d][1,3]dioxole-4-carboxamido)propyl)(phenethyl)carbamate 26a (60 mg, crude) was added TFA (1.0 mL) and the reaction mixture was stirred at 50° C. for 1 h. The resulting mixture was concentrated under vacuum and basified to pH >8 with NH₃.H₂O. The crude sample was purified by prep-HPLC [Xbridge Prep C18 OBD column, 5 μm, 19*150 mm; mobile phase, Water (0.03% NH₄OH) and CH₃CN (10% up to 34% in 10 min); Detector, UV 220&254 nm]: to afford (1S,2R,3S,4R)-4-(4-amino-5-bromo-7H-pyrrolo[2,3-d]pyrimidin-7-yl)-2,3-dihydroxy-N-(3-(phenethylamino)propyl) cyclopentane-1-carboxamide 452 (23.2 mg, 49% over 2 steps) as a white solid. ¹H NMR (400 MHZ, DMSO-d₆) δ 8.09 (s, 1H), 8.00 (t, J=5.6 Hz, 1H), 7.63 (s, 1H), 7.31-7.23 (m, 2H), 7.23-7.12 (m, 3H), 6.85-6.45 (m, 2H), 5.02-4.89 (m, 3H), 4.16 (dt, J=7.6, 5.5 Hz, 1H), 4.06-3.98 (m, 1H), 3.12 (q, J=6.6 Hz, 2H), 2.77-2.66 (m, 5H), 2.58-2.52 (m, 2H), 2.56-2.51 (m, 1H), 1.88-1.80 (m, 1H), 1.57-1.33 (m, 2H). LCMS [conditions: Cortecs C_{18+, 50*3.0} mm, 2.7 μm, Mobile Phase A: Water/0.05% TFA, Mobile Phase B: Acetonitrile, 5%-95% B-3 min.lcm, 1.50 mL/min, ES, m/z]: T_{R}=0.65 min; [M+H]⁺: 517, 519. Chiral SFC [Column: SC 100×4.6 mm 3.0 μm; Co Solvent: B: MeOH (20 mM NH₃); Start Conc. of Pump B: 30.0%; Total Flow: 3.00 mL/min; BPR Pressure: 15.00 MPa; BPR Temperature: 50° C.]: T_{R}=2.55 min; dr >99:1.

### Synthesis of (1S,2R,3S,4R)-4-(4-amino-5-bromo-7H-pyrrolo[2,3-d]pyrimidin-7-yl)-2,3-dihydroxy-N-(((S)-piperidin-3-yl)methyl) cyclopentane-1-carboxamide (453)

### tert-Butyl-(R)-3-(((3αR,4S,6R,6αS)-6-(5-bromo-4-chloro-7H-pyrrolo[2,3-d]pyrimidin-7-yl)-2,2-dimethyltetrahydro-4H-cyclopenta[d][1,3]dioxole-4-carboxamido) methyl) piperidine-1-carboxylate (28a)

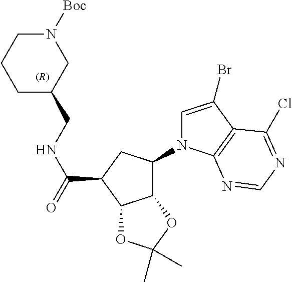

Methyl(3αR,4S,6R,6αS)-6-{5-bromo-4-chloropyrrolo[2,3-d]pyrimidin-7-yl}-2,2-dimethyl-tetrahydro-3αH-cyclopenta[d][1,3]dioxole-4-carboxylate 23a (200 mg, 0.46 mmol, 1.0 eq) and tert-butyl-(3R)-3-(aminomethyl) piperidine-1-carboxylate (149 mg, 0.70 mmol, 1.5 eq) were dissolved in toluene (3.0 mL) and the mixture was to cooled to 0° C. and then AlMe3 (1 M in toluene, 0.93 mL, 0.93 mmol, 2.0 eq) was added and the reaction mixture was heated to 80° C. and stirred for 4 h. The resulting mixture was concentrated under vacuum. The crude residue was purified by silica gel column chromatography (12/1 DCM/MeOH) to afford tert-butyl-(3R)-3-({[(3αR,45,6R,6αS)-6-{5-bromo-4-chloropyrrolo[2,3-d]pyrimidin-7-yl}-2,2-dimethyl-tetrahydro-3αH-cyclopenta[d][1,3]dioxol-4-yl]formamido}methyl) piperidine-1-carboxylate 28a (120 mg, 42%) as an off-white solid. LCMS [conditions: Kinetex XB-C18, 3.0*30 mm, 1.7 μm, Mobile Phase A: Water/0.1% FA, Mobile Phase B: Acetonitrile/0.05% FA, 5%-100% B-2.5 min-P.lcm, 1.50 mL/min, ES, m/z]: T_{R}=1.34 min; [M+H]⁺: 614, 616.

### tert-Butyl-(R)-3-(((3αR,4S,6R,6αS)-6-(4-amino-5-bromo-7H-pyrrolo[2,3-d]pyrimidin-7-yl)-2,2-dimethyltetrahydro-4H-cyclopenta[d][1,3]dioxole-4-carboxamido) methyl) piperidine-1-carboxylate (29a)

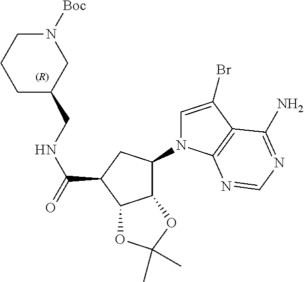

To tert-butyl-(3R)-3-({[(3αR,4S,6R,6αS)-6-{5-bromo-4-chloropyrrolo[2,3-d]pyrimidin-7-yl}-2,2-dimethyl-tetrahydro-3αH-cyclopenta[d][1,3]dioxol-4-yl]formamido}methyl) piperidine-1-carboxylate 28a (120 mg, 0.20 mmol, 1.0 eq) was added NH₃ (7M in MeOH, 3.0 mL) and the reaction mixture was stirred at 75° C. for 8 h. The resulting mixture was concentrated under vacuum to afford tert-butyl (3R)-3-({[(3αR,4S,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]formamido}methyl) piperidine-1-carboxylate 29a (100 mg, crude) as a light yellow solid. The crude product was used in the next step without further purification.

### (1S,2R,3S,4R)-4-(4-Amino-5-bromo-7H-pyrrolo[2,3-d]pyrimidin-7-yl)-2,3-dihydroxy-N-(((S)-piperidin-3-yl)methyl) cyclopentane-1-carboxamide (453)

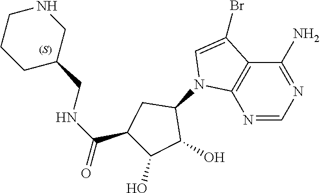

tert-Butyl-(3R)-3-({[(3αR,4S,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]formamido}methyl) piperidine-1-carboxylate 29a (100 mg, 0.17 mmol, 1.0 eq) was dissolved in TFA (3.0 mL) at room temperature and the reaction mixture was stirred for 2 h. The resulting mixture was concentrated under reduced pressure and the residue was basified to pH>10 with NH₃.H₂O. The resulting mixture was purified by Prep-HPLC [condition: column, Xbridge Prep C18 OBD column, 5 μm, 19*150 mm; mobile phase, Water (0.03% NH₄OH) and CH₃CN (10% up to 34% in 10 min); Detector, UV 220&254 nm]: to afford (1S,2R,3S,4R)-4-{4-amino-5-bromopyrrolo[2,3-d]pyrimidin-7-yl}-2,3-dihydroxy-N-[(3S)-piperidin-3-ylmethyl]cyclopentane-1-carboxamide 453 (32.4 mg, 42%) as an off-white solid. ¹H NMR (400 MHZ, DMSO-d₆) δ 8.06 (s, 1H), 7.58 (s, 1H), 4.97-4.81 (m, 1H), 4.15-4.10 (m, 1H), 4.03-3.98 (m, 1H), 3.05-2.84 (m, 4H), 2.77-2.69 (m, 1H), 2.48-2.41 (m, 1H), 2.40-2.18 (m, 2H), 1.90-1.77 (m, 1H), 1.75-1.32 (m, 4H), 1.09-0.95 (m, 1H). LCMS [conditions: ZORBAX SB-Aq, 50*4.6 mm, 1.8 μm, Mobile Phase A: Water/0.02% TFA, Mobile Phase B: Acetonitrile, 5%-95% B-3 min-1.5.lcm, 1.50 mL/min, ES, m/z]: T_{R}=0.92 min; [M+H]⁺: 453, 455. Chiral SFC [conditions: Column: SC 100×4.6 mm 3.0 μm; Co Solvent: B: MeOH (20 mM NH₃); Start Conc. of Pump B: 45.0%; Total Flow: 3.00 mL/min; BPR Pressure: 15.00 MPa; BPR Temperature: 50° C.]: T_{R}=1.46 min (major peak); dr=92:8.

### Synthesis of (1S,2R,35,4R)-4-(4-amino-5-bromo-7H-pyrrolo[2,3-d]pyrimidin-7-yl)-2,3-dihydroxy-N-(((R)-piperidin-3-yl)methyl) cyclopentane-1-carboxamide (454)

### tert-Butyl-(S)-3-(((3αR,4S,6R,6αS)-6-(5-bromo-4-chloro-7H-pyrrolo[2,3-d]pyrimidin-7-yl)-2,2-dimethyltetrahydro-4H-cyclopenta[d][1,3]dioxole-4-carboxamido) methyl) piperidine-1-carboxylate (30a)

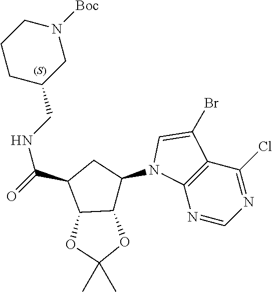

To a mixture of methyl(3αR,4S,6R,6αS)-6-(5-bromo-4-chloro-7H-pyrrolo[2,3-d]pyrimidin-7-yl)-2,2-dimethyltetrahydro-4H-cyclopenta[d][1,3]dioxole-4-carboxylate 23a (200 mg, 0.464 mmol, 1.0 eq) and tert-butyl-(3S)-3-(aminomethyl) piperidine-1-carboxylate (149 mg, 0.70 mmol, 1.5 eq) in toluene (3.0 mL) at 0° C. was added AlMe₃ (1M in toluene, 0.93 mL, 0.93 mmol, 2.0 eq) and the reaction mixture was heated at 80° C. for 4 h. The resulting mixture was concentrated under vacuum and the crude residue was purified by silica gel column chromatography (12/1 DCM/MeOH) to afford tert-butyl-(S)-3-(((3αR,4S,6R,6αS)-6-(5-bromo-4-chloro-7H-pyrrolo[2,3-d]pyrimidin-7-yl)-2,2-dimethyltetrahydro-4H-cyclopenta[d][1,3]dioxole-4-carboxamido) methyl) piperidine-1-carboxylate 30a (100 mg, 35%) as an off-white solid. LCMS [conditions: Kinetex XB-C18, 3.0*30 mm, 1.7 μm, Mobile Phase A: Water/0.1% FA, Mobile Phase B: Acetonitrile/0.05% FA, 5%-100% B-2.5 min-P.Icm, 1.50 mL/min, ES, m/z]: T_{R}=1.35 min; [M+H]⁺: 614, 616.

### tert-Butyl-(S)-3-(((3αR,4S,6R,6αS)-6-(4-amino-5-bromo-7H-pyrrolo[2,3-d]pyrimidin-7-yl)-2,2-dimethyltetrahydro-4H-cyclopenta[d][1,3]dioxole-4-carboxamido) methyl) piperidine-1-carboxylate (31a)

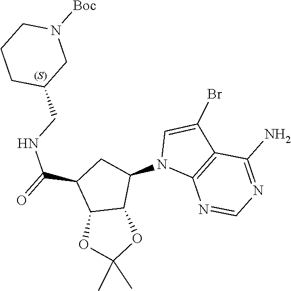

To tert-butyl-(S)-3-(((3αR,4S,6R,6αS)-6-(5-bromo-4-chloro-7H-pyrrolo[2,3-d]pyrimidin-7-yl)-2,2-dimethyltetrahydro-4H-cyclopenta[d][1,3]dioxole-4-carboxamido) methyl) piperidine-1-carboxylate 30a (110 mg, 0.18 mmol, 1.0 eq) was added in NH₃ (7M in MeOH, 3.0 mL) and the reaction mixture was stirred at 75° C. for 8 h. The resulting mixture was concentrated under vacuum to afford tert-butyl (S)-3-(((3αR,4S,6R,6αS)-6-(4-amino-5-bromo-7H-pyrrolo[2,3-d]pyrimidin-7-yl)-2,2-dimethyltetrahydro-4H-cyclopenta[d][1,3]dioxole-4-carboxamido) methyl) piperidine-1-carboxylate 31a (74 mg, crude) as an off-white solid. The crude product was used in the next step directly without further purification. LCMS [conditions: L-column3 ODS, 50*3.0 mm, 3.0 μm, Mobile Phase A: Water (0.05% ammonia water), Mobile Phase B: Acetonitrile, 5%-100% B-2.5 min, 1.20 mL/min, ES, m/z]: T_{R}=1.35 min; [M+H]⁺: 593, 595.

### (1S,2R,3S,4R)-4-(4-Amino-5-bromo-7H-pyrrolo[2,3-d]pyrimidin-7-yl)-2,3-dihydroxy-N-(((R)-piperidin-3-yl)methyl) cyclopentane-1-carboxamide (454)

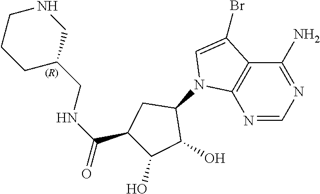

To tert-butyl-(S)-3-(((3αR,4S,6R,6αS)-6-(4-amino-5-bromo-7H-pyrrolo[2,3-d]pyrimidin-7-yl)-2,2-dimethyltetrahydro-4H-cyclopenta[d][1,3]dioxole-4-carboxamido) methyl) piperidine-1-carboxylate 31a (74 mq, crude) was added TFA (1.0 mL) and the reaction mixture was stirred at 50° C. for 1 h. The resulting mixture was concentrated under vacuum and basified to pH>8 with NH₃.H₂O. The crude residue was purified by prep-HPLC [column, Xbridge Prep C18 OBD column, 5 μm, 19*150 mm; mobile phase, water (0.03% NH₄OH) and CH₃CN (10% up to 34% in 10 min); Detector, UV 220&254 nm]: to afford (1S,2R,3S,4R)-4-{4-amino-5-bromopyrrolo[2,3-d]pyrimidin-7-yl}-2,3-dihydroxy-N-[(3R)-piperidin-3-ylmethyl]cyclopentane-1-carboxamide 454 (36.4 mg, 64%) as a white solid. ¹H NMR (300 MHZ, DMSO-d₆) δ 8.07 (s, 1H), 7.59 (s, 1H), 4.92 (q, J=8.6 Hz, 1H), 4.13 (dd, J=7.6, 5.2 Hz, 1H), 4.01 (t, J=4.7 Hz, 1H), 3.00-2.87 (m, 4H), 2.79-2.66 (m, 1H), 2.47-2.18 (m, 3H), 1.91-1.75 (m, 1H), 1.75-1.51 (m, 3H), 1.44-1.33 (m, 1H), 1.12-0.95 (m, 1H). LCMS [conditions: ZORBAX SB-Aq, 50*4.6 mm, 1.8 μm, Mobile Phase A: Water/0.02% TFA, Mobile Phase B: Acetonitrile, 5%-95% B-3 min-1.5.lcm, 1.50 mL/min, ES, m/z]: T_{R}=0.93 min; [M+H]⁺: 453,455. Chiral SFC [condition: Column: SC 100×4.6 mm 3.0 μm; Co Solvent: B: MeOH (20 mM NH₃); Start Conc. of Pump B: 40.0%; Total Flow: 3.00 mL/min; BPR Pressure: 15.00 MPa; BPR Temperature: 50° C.]: T_{R}=1.85 min (major peak); dr=92.8:7.2.

### Synthesis of (1R,2S,3R,5R)-3-(4-amino-5-bromo-7H-pyrrolo[2,3-d]pyrimidin-7-yl)-5-(((((S)-piperidin-3-yl)methyl) amino) methyl) cyclopentane-1,2-diol (455)

### tert-Butyl-(S)-3-(((tert-butoxycarbonyl)(((3αR,4R,6R,6αS)-6-(4-chloro-7H-pyrrolo[2,3-d]pyrimidin-7-yl)-2,2-dimethyltetrahydro-4H-cyclopenta[d][1,3]dioxol-4-yl)methyl) amino) methyl) piperidine-1-carboxylate (145a)

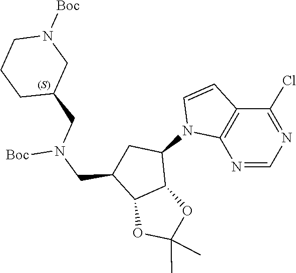

To tert-butyl-(3R)-3-(aminomethyl) piperidine-1-carboxylate (0.13 g, 0.61 mmol, 1.3 equiv) in DCM (3.0 mL) and AcOH (0.28 g, 4.7 mmol, 10.0 eq) was added NaBH (OAc)₃ (0.19 g, 0.90 mmol, 2.0 eq), at room temperature and the resulting mixture was stirred for 30 min at room temperature. To this mixture was added (3αR,4S,6R,6αS)-6-{4-chloropyrrolo[2,3-d]pyrimidin-7-yl}-2,2-dimethyl-tetrahydro-3αH-cyclopenta[d][1,3]dioxole-4-carbaldehyde 77a (0.15 g, 0.50 mmol, 1.0 eq) in DCM (1.0 mL) and the reaction mixture was stirred for 1 h at room temperature. After this time, Boc₂O (0.15 g, 0.70 mmol, 1.5 eq) and TEA (0.70 g, 7.0 mmol, 15 eq) were added and the reaction mixture was stirred for 30 min at room temperature. The resulting mixture was concentrated under reduced pressure and the crude product was purified by Prep-HPLC [Column: Xtimate C18 50×250 mm, 10 μm; Mobile Phase A: Water (0.05% NH₃·H₂O), Mobile Phase B: ACN; Flow rate: 90 mL/min; Gradient: 90% B in 15 min; Wave Length: 220 nm]: to afford tert-butyl-(3S)-3-[{{[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}(tert-butoxycarbonyl) amino) methyl]piperidine-1-carboxylate 145a (0.13 g, 45%) as a yellow solid. LCMS [XBridge C18, 50*3.0 mm, 3.5 μm; Mobile Phase A: Water/5 mM NH₄HCO₃, Mobile Phase B: MeOH, 10%-95% B-5 min (+−), 1.50 mL/min, ES, m/z]: T_{R}=4.13 min; [M+H]⁺: 620.

### tert-Butyl-(S)-3-(((((3αR,4R,6R,6αS)-6-(5-bromo-4-chloro-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino) methyl) piperidine-1-carboxylate (146a)

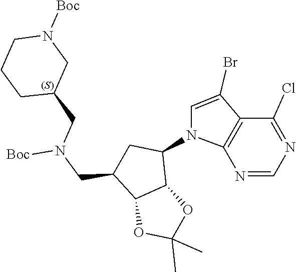

To tert-butyl-(3S)-3-[{{[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}(tert-butoxycarbonyl) amino) methyl]piperidine-1-carboxylate 145a (0.13 mg, 0.20 mmol, 1.0 eq) in DMF (3.0 mL) was added NBS (45 mg, 0.25 mmol, 1.2 eq) at room temperature and the reaction mixture was stirred for 2 h at room temperature. The reaction was quenched by the addition of water (5.0 mL) at room temperature and the resulting mixture was extracted with AcOEt (3×10 mL). The combined organic phases were concentrated under reduced pressure and the crude residue was purified by Prep-TLC(95:5 DCM: MeOH) to afford tert-butyl-(35)-3-[{{[(3αR,4R,6R,6αS)-6-{5-bromo-4-chloropyrrolo[2,3-d]pyrimidin-7-yl}-2,2-dimethyl-tetrahydro-3αH-cyclopenta[d][1,3]dioxol-4-yl]methyl}(tert-butoxycarbonyl) amino) methyl]piperidine-1-carboxylate 146a (0.11 g, 75%) as a yellow solid. LCMS [XBridge C18, 50*3.0 mm, 3.5 μm; Mobile Phase A: Water/5 mM NH₄HCO₃, Mobile Phase B: MeOH, 10%-95% B-5 min (+), 1.50 mL/min, ES, m/z]: T_{R}=4.41 min; [M+H]⁺: 698, 700.

### tert-Butyl-(R)-3-(((((3αR,4R,6R,6αS)-6-(4-amino-5-bromo-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino) methyl) piperidine-1_carhow/late/1/72)

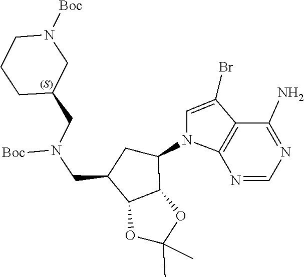

To tert-butyl-(3S)-3-[{{[(3αR,4R,6R,6αS)-6-{5-bromo-4-chloropyrrolo[2,3-d]pyrimidin-7-yl}-2,2-dimethyl-tetrahydro-3αH-cyclopenta[d][1,3]dioxol-4-yl]methyl}(tert-butoxycarbonyl) amino) methyl]piperidine-1-carboxylate 146a (0.11 g, 0.15 mmol, 1.0 eq) was added NH₃ (7M in MeOH, 3.0 mL) at room temperature and the reaction mixture was stirred overnight at 75° C. The resulting mixture was concentrated under reduced pressure to afford tert-butyl-(3S)-3-[{{[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino) methyl]piperidine-1-carboxylate 147a (80 mg, crude) as a yellow solid. LCMS [XBridge C18, 50*3.0 mm, 3.5 μm; Mobile Phase A: Water/5 mM NH₄HCO₃, Mobile Phase B: MeOH, 10%-95% B-5 min (+−), 1.50 mL/min, ES, m/z]: T_{R}=3.98 min; [M+H]⁺: 679, 681.

### (1R,2S,3R,5R)-3-(4-Amino-5-bromo-7H-pyrrolo[2,3-d]pyrimidin-7-yl)-5-(((((S)-piperidin-3-yl)methyl)amino)methyl)cyclopentane-1,2-diol (455)

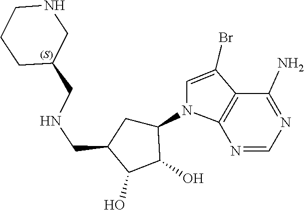

To tert-butyl-(3S)-3-[{{[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino) methyl]piperidine-1-carboxylate 147a (80 mg, 0.10 mmol, 1.0 eq) was added TFA (3.0 mL) at room temperature and the reaction mixture was stirred at 50° C. for 1 h. The resulting mixture was concentrated under reduced pressure and basified to pH 10 with NH₃.H₂O. The crude residue was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 20% B to 50% B in 7 min, 50% B; Wave Length: 220 nm]: to afford (1R,2S,3R,5R)-3-{4-amino-5-bromopyrrolo[2,3-d]pyrimidin-7-yl}-5-({[(3S)-piperidin-3-ylmethyl]amino}methyl) cyclopentane-1,2-diol 327 (20.5 mg, 39%) as an off-white solid. ¹H NMR (400 MHZ, DMSO-d₆+D₂O) 88.08 (s, 1H), 7.57 (s, 1H), 4.93-4.77 (m, 1H), 4.29-4.08 (m, 1H), 3.77-3.72 (m, 1H), 3.46-2.48 (m, 5H), 2.43-2.25 (m, 2H), 2.23-1.56 (m, 5H), 1.52-1.36 (m, 2H), 1.23-0.96 (m, 1H). LCMS [conditions Atlantis T3, 100*4.6 mm, 3 μm, Mobile Phase A: Water/0.02% TFA, Mobile Phase B: Acetonitrile, 0%-30%-95% B-7 min, 1.20 mL/min, ES, m/z]: T_{R}=3.33 min; [M+H]⁺: 439, 441. Chiral-SFC [Column: IG 100×4.6 mm 3.0 μm, Solvent B: MeOH (20 mM NH₃), Oven Temperature: 35° C., Total Flow: 3.00 mL/min. Gradient 50% solvent B for 4 mins]: T_{R}=1.95 min, single peak.

### Synthesis of (1R,2S,3R,5R)-3-(4-amino-5-bromo-7H-pyrrolo[2,3-d]pyrimidin-7-yl)-5-(((((R)-piperidin-3-yl)methyl) amino) methyl) cyclopentane-1,2-diol (456)

### tert-Butyl-(R)-3-(((tert-butoxycarbonyl)(((3αR,4R,6R,6αS)-6-(4-chloro-7H-pyrrolo[2,3-d]pyrimidin-7-yl)-2,2-dimethyltetrahydro-4H-cyclopenta[d][1,3]dioxol-4-yl)methyl) amino) methyl) piperidine-1-carboxylate (148a)

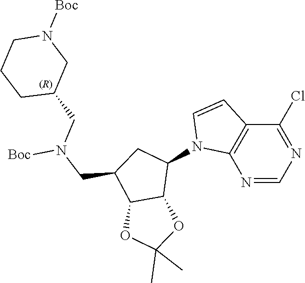

To tert-butyl-(3S)-3-(aminomethyl) piperidine-1-carboxylate (0.13 mg, 0.60 mmol, 1.3 eq), NaBH (OAc)₃ (0.19 g, 0.90 mmol, 2.0 eq) in DCM (3.0 mL) was added AcOH (0.27 g, 4.60 mmol, 10 eq) at room temperature and the resulting mixture was stirred for 30 min at room temperature. After this time, (3αR,4S,6R,6αS)-6-{4-chloropyrrolo[2,3-d]pyrimidin-7-yl}-2,2-dimethyl-tetrahydro-3αH-cyclopenta[d][1,3]dioxole-4-carbaldehyde 77a (0.15 g, 0.50 mmol, 1.0 eq) in DCM (1.0 mL) was added and the reaction mixture was stirred for 1 h at room temperature. To the resulting mixture was then added Boc₂O (0.15 g, 0.70 mmol, 1.5 eq) and TEA (0.70 g, 7.0 mmol, 15 eq) and the reaction mixture was stirred for 30 min at room temperature. The resulting mixture was concentrated under reduced pressure and the crude product was purified by Prep-HPLC [Column: Xtimate C18 50×250 mm, 10 μm; Mobile Phase A: Water (0.05% NH₃.H₂O), Mobile Phase B: ACN; Flow rate: 90 mL/min; Gradient: 90% B in 15 min; Wave Length: 220 nm]: to afford tert-butyl-(3R)-3-[{{[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}(tert-butoxycarbonyl) amino) methyl]piperidine-1-carboxylate 148a (0.14 g, 50%) as a yellow solid. LCMS [Xbridge Shield RP18, 50*3.0 mm; Mobile Phase A: Water/0.05% Ammonia, Mobile Phase B: Acetonitrile, 5%-95% B-5 min (+−), 1.20 mL/min, ES, m/z]: T_{R}=3.70 min; [M+H]⁺: 620.

### tert-Butyl-(R)-3-(((((3αR,4R,6R,6αS)-6-(5-bromo-4-chloro-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino) methyl) piperidine-1-carboxylate (149a)

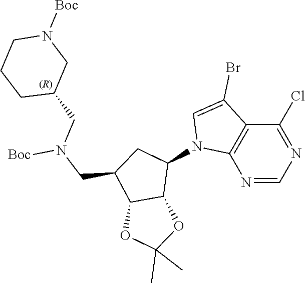

To tert-butyl-(3R)-3-[{{[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}(tert-butoxycarbonyl) amino) methyl]piperidine-1-carboxylate 148a (0.14 g, 0.20 mmol, 1.0 eq) in DMF (3.0 mL) was added NBS (50 mg, 0.30 mmol, 1.2 eq) at room temperature and the reaction mixture was stirred for 2 h at room temperature. The reaction was quenched by the addition of water (5.0 mL) at room temperature and the resulting mixture was extracted with AcOEt (3×10 mL). The combined organic extracts were concentrated under reduced pressure and purified by Prep-TLC(95:5 DCM/MeOH) to afford tert-butyl-(3R)-3-[{{[(3αR,4R,6R,6αS)-6-{5-bromo-4-chloropyrrolo[2,3-d]pyrimidin-7-yl}-2,2-dimethyl-tetrahydro-3αH-cyclopenta[d][1,3]dioxol-4-yl]methyl}(tert-butoxycarbonyl) amino) methyl]piperidine-1-carboxylate 149a (0.14 g, 86%) as a yellow solid. LCMS [XBridge C18, 50*3.0 mm, 3.5 μm; Mobile Phase A: Water/5 mM NH₄HCO₃, Mobile Phase B: MeOH, 10%-95% B-5 min (+), 1.00 mL/min, ES, m/z]: T_{R}=4.32 min; [M+H]⁺: 698, 700

### tert-Butyl-(R)-3-(((((3αR,4R,6R,6αS)-6-(4-amino-5-bromo-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino) methyl) piperidine-1-carboxylate (150a)

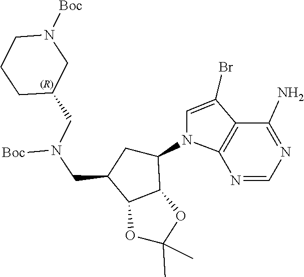

To tert-butyl-(3R)-3-[{{[(3αR,4R,6R,6αS)-6-{5-bromo-4-chloropyrrolo[2,3-d]pyrimidin-7-yl}-2,2-dimethyl-tetrahydro-3αH-cyclopenta[d][1,3]dioxol-4-yl]methyl}(tert-butoxycarbonyl) amino) methyl]piperidine-1-carboxylate 149a (0.14 g, 0.20 mmol, 1.0 eq) was added NH₃ (7M in MeOH, 3.0 mL) at room temperature and the reaction mixture was stirred at 75° C. overnight and the resulting mixture was concentrated under reduced pressure to afford crude tert-butyl-(3R)-3-[{{[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino) methyl]piperidine-1-carboxylate 150a (0.12 g, crude) as a yellow solid. LCMS [XBridge C18, 50*3.0 mm, 3.5 μm; Mobile Phase A: Water/5 mM NH₄HCO₃, Mobile Phase B: MeOH, 10%-95% B-5 min (+−), 1.00 ml/min, ES, m/z]: T_{R}=3.98 min; [M+H]⁺: 679, 681.

### (1R,2S,3R,5R)-3-(4-Amino-5-bromo-7H-pyrrolo[2,3-d]pyrimidin-7-yl)-5-(((((R)-piperidin-3-yl)methyl) amino) methyl) cyclopentane-1,2-diol (456)

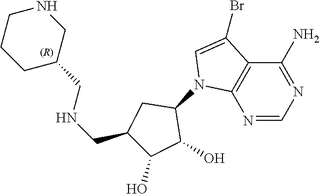

To tert-butyl-(3R)-3-[{{[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino) methyl]piperidine-1-carboxylate 150a (0.12 g, 0.20 mmol, 1.0 eq) was added TFA (3.0 mL) at room temperature and the reaction mixture was stirred at 50° C. for 1 h. The resulting mixture was concentrated under reduced pressure and the crude residue was basified to pH=10 with NH₃.H₂O and purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 20% B to 50% B in 7 min, 50% B; Wave Length: 220 nm]: to afford (1R,2S,3R,5R)-3-{4-amino-5-bromopyrrolo[2,3-d]pyrimidin-7-yl}-5-({[(3R)-piperidin-3-ylmethyl]amino}methyl) cyclopentane-1,2-diol 456 (34.8 mg, 45%) as an off-white solid. ¹H NMR (400 MHZ, DMSO-d₆+D₂O) 8 8.07 (s, 1H), 7.64-7.46 (m, 1H), 4.97-4.77 (m, 1H), 4.29-4.06 (m, 1H), 3.89-3.56 (m, 2H), 3.51-3.38 (m, 1H), 3.36-2.82 (m, 2H), 2.73-2.59 (m, 1H), 2.57-2.48 (m, 1H), 2.46-2.32 (m, 2H), 2.26-2.11 (m, 1H), 2.07-1.98 (m, 1H), 1.81-1.70 (m, 1H), 1.67-1.56 (m, 2H), 1.51-1.40 (m, 2H), 1.18-0.93 (m, 1H). LCMS [conditions Atlantis T3, 100*4.6 mm, 3 μm, Mobile Phase A: Water/0.02% TFA, Mobile Phase B: Acetonitrile, 0%-30%-95% B-7 min, 1.20 mL/min, ES, m/z]: T_{R}=3.39 min; [M+H]⁺: 439, 441. Chiral-SFC [Column: IG 100×4.6 mm 3.0 μm, Solvent B: MeOH (20 mM NH₃), Oven Temperature: 35° C., Total Flow: 3.00 ml/min. Gradient 50% solvent B for 4 mins]: T_{R}=1.21 min, single peak.

### Synthesis of ((1R,2S,3R,5R)-3-(4-amino-5-bromo-7H-pyrrolo[2,3-d]pyrimidin-7-yl)-5-(((3-((3-phenoxyphenethyl)amino)propyl)amino)methyl)cyclopentane-1,2-diol (457)

### tert-Butyl-(3-(1,3-dioxoisoindolin-2-yl)propyl)(3-phenoxyphenethyl)carbamate (50a)

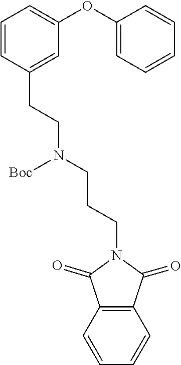 [SMILES: CC(C)(C)OC(=O)N(CCCN1C(=O)c2ccccc2C1=O)CCc1cccc(Oc2ccccc2)c1]

To a stirred mixture of tert-butyl-(3-hydroxypropyl)(3-phenoxyphenethyl)carbamate 47a (1.4 g, 3.77 mmol, 1.0 eq), phthalimide (1.11 g, 7.54 mmol, 2.0 eq) and PPh₃ (1.98 g, 7.54 mmol, 2.0 eq) in THF (40 mL) were added DIAD (1.52 g, 7.54 mmol, 2.0 eq) dropwise at 0° C. under nitrogen and the reaction mixture was stirred for 2 hours at 0° C. The reaction was quenched by the addition of water (20 mL) at room temperature and the resulting mixture was extracted with EtOAc (150 mL). The organic layer was washed with brine (50 mL), dried over anhydrous Na₂SO₄ and filtered. The filtrate was concentrated under reduced pressure and the crude residue was purified by silica gel column chromatography (5:1 Petroleum ether/AcOEt) to afford tert-butyl-(3-(1,3-dioxoisoindolin-2-yl)propyl)(3-phenoxyphenethyl)carbamate 50a (1.8 g, 95%) as a white solid. LCMS [conditions: Xbridge Shield RP18, 50*3.0 mm, 3.5 μm, Mobile Phase A: Water/0.05% ammonia water, Mobile Phase B: Acetonitrile, 5%-100% B-2.0 min (+), 1.20 mL/min, ES, m/z]: T_{R}=1.52 min; [M+H]⁺: 501.

### tert-Butyl(3-aminopropyl)(3-phenoxyphenethyl)carbamate (51a)

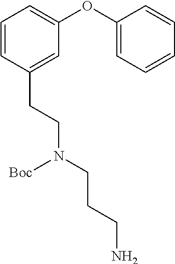 [SMILES: CC(C)(C)OC(=O)N(CCCN)CCc1cccc(Oc2ccccc2)c1]

To a solution of tert-butyl-(3-(1,3-dioxoisoindolin-2-yl)propyl)(3-phenoxyphenethyl)carbamate 50a (1.80 g, 3.60 mmol, 1.0 eq) in EtOH (20 mL) was added NH₂NH₂.H₂O (1.06 g, 18.0 mmol, 5.0 eq, 85%), dropwise, at room temperature under nitrogen and the reaction mixture was stirred for at 80° C. 3 h. The resulting mixture was allowed to cool to room temperature and concentrated in vacuum. The resulting residue was partitioned between water (20 mL) and DCM (50 mL) and the organic layer was washed with brine (20 mL), dried over anhydrous Na₂SO₄, filtered and concentrated in vacuum to afford tert-butyl-(3-aminopropyl)(3-phenoxyphenethyl)carbamate 51a (1.3 g, 98%) as a light yellow oil, which was used to next step without further purification. LCMS [conditions: Kinetex XB-C18, 3.0*30 mm, 1.7 μm, Mobile Phase A: Water/0.1% FA, Mobile Phase B: Acetonitrile/0.05% FA, 5%-100% B-1.2 min (+), 1.50 mL/min, ES, m/z]: T_{R}=0.58 min; [M+H]⁺: 371.

### tert-Butyl-(3-((tert-butoxycarbonyl)(((3αR,4R,6R,6αS)-6-(4-chloro-7H-pyrrolo[2,3-d]pyrimidin-7-yl)-2,2-dimethyltetrahydro-4H-cyclopenta[d][1,3]dioxol-4-yl)methyl) amino)propyl)(3-phenoxyphenethyl)carbamate (52a). (WO2020033288)

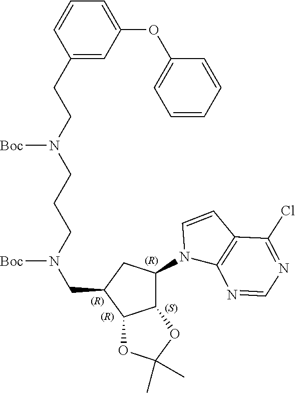

To (3αR,4S,6R,6αS)-6-(4-chloro-7H-pyrrolo[2,3-d]pyrimidin-7-yl)-2,2-dimethyltetrahydro-4H-cyclopenta[d][1,3]dioxole-4-carbaldehyde 77 (140 mg, 0.44 mmol, 1.0 eq) and tert-butyl-(3-aminopropyl)(3-phenoxyphenethyl)carbamate 51a (177 mg, 0.48 mmol, 1.1 eq) in DCM (5.0 mL) was added NaBH (AcO)₃ (30.0 mg, 1.31 mmol, 3.0 eq) at room temperature under nitrogen and the reaction mixture was stirred for 1 h at room temperature. To this mixture was added TEA (220 mg, 2.18 mmol, 5.0 eq) and Boc₂O (142 mg, 0.65 mmol, 1.5 eq), at room temperature. The reaction mixture was stirred at room temperature for 3 hours and was then quenched by the addition of sat. NH₄Cl (aq.)(5 mL) and then diluted with DCM (20 mL). The organic layer was washed with brine (10 mL), dried over anhydrous Na₂SO₄, filtered and concentrated under reduced pressure. The crude residue was purified by silica gel column chromatography (5:1 Petroleum ether/AcOEt) to afford tert-butyl-(3-((tert-butoxycarbonyl)(((3αR,4R,6R,6αS)-6-(4-chloro-7H-pyrrolo[2,3-d]pyrimidin-7-yl)-2,2-dimethyltetrahydro-4H-cyclopenta[d][1,3]dioxol-4-yl)methyl) amino)propyl)(3-phenoxyphenethyl)carbamate 52a (140 mg, 41%) as a light yellow oil. ¹H NMR (400 MHZ, Chloroform-d) 8 8.65 (s, 1H), 7.39-7.30 (m, 3H), 7.25 (d, J=7.9 Hz, 1H), 7.12 (t, J=7.4 Hz, 1H), 7.02 (d, J=8.0 Hz, 2H), 6.94 (s, 1H), 6.87 (d, J=7.5 Hz, 2H), 6.66 (d, J=3.6 Hz, 1H), 5.07-4.89 (m, 2H), 4.64-4.55 (m, 1H), 3.52-3.33 (m, 4H), 3.26-3.17 (m, 5H), 2.89-2.76 (m, 2H), 2.49-2.33 (m, 2H), 1.85-1.72 (m, 2H), 1.56 (s, 3H), 1.45 (s, 18H), 1.29 (s, 3H). LCMS [conditions: HPH-C18, 50*3.0 mm, 2.7 μm, Mobile Phase A: Water/0.05% ammonia water, Mobile Phase B: Acetonitrile, 5%-100% B-2.0 min (+), 1.20 mL/min, ES, m/z]: T_{R}=1.61 min; [M+H]⁺: 776, 778.

### tert-Butyl(3-((((3αR,4R,6R,6αS)-6-(5-bromo-4-chloro-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(3-phenoxyphenethyl)carbamate (53a)

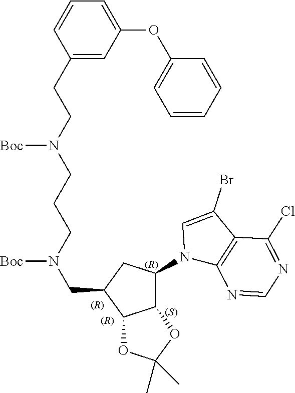

To a solution of tert-butyl-(3-((tert-butoxycarbonyl)(((3αR,4R,6R,6αS)-6-(4-chloro-7H-pyrrolo[2,3-d]pyrimidin-7-yl)-2,2-dimethyltetrahydro-4H-cyclopenta[d][1,3]dioxol-4-yl)methyl) amino)propyl)(3-phenoxyphenethyl)carbamate 52a (50 mg, 0.064 mmol, 1.0 eq) in DMF (2.0 mL) was added NBS (11.4 mg, 0.064 mmol, 1.0 eq) at room temperature under nitrogen and the reaction mixture was stirred for 1 h at room temperature. The reaction was quenched by the addition of sat. NaHCO₃(aq.)(10 mL) at room temperature and extracted with EtOAc (20 mL). The organic layer was washed with brine (10 mL), dried over anhydrous Na₂SO₄. After filtration, the filtrate was concentrated under reduced pressure and the crude residue was purified by silica gel column chromatography (5:1 Petroleum ether/AcOEt) to afford tert-butyl (3-((((3αR,4R,6R,6αS)-6-(5-bromo-4-chloro-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(3-phenoxyphenethyl)carbamate 53a (50 mg, 91%) as a light yellow solid. ¹H NMR (400 MHZ, Chloroform-d) 8 8.64 (s, 1H), 7.41-7.32 (m, 3H), 7.26 (d, J=8.1 Hz, 1H), 7.12 (t, J=7.4 Hz, 1H), 7.05-6.99 (m, 2H), 6.94 (d, J=7.3 Hz, 1H), 6.87 (d, J=6.9 Hz, 2H), 5.08-4.87 (m, 2H), 4.61-4.52 (m, 1H), 3.51-3.32 (m, 4H), 3.16-3.22 (m, 5H), 2.86-2.78 (m, 2H), 2.51-2.38 (m, 2H), 1.83-1.74 (m, 2H), 1.56 (s, 3H), 1.46 (s, 18H), 1.32-1.25 (m, 3H). LCMS [conditions: Xbridge Shield RP18, 50*3.0 mm, 3.5 μm, Mobile Phase A: Water/0.05% ammonia water, Mobile Phase B: Acetonitrile, 5%-100% B-2.0 min (+), 1.20 mL/min, ES, m/z]: T_{R}=1.72 min; [M+H]⁺: 854, 856.

### tert-Butyl(3-((((3αR,4R,6R,6αS)-6-(4-amino-5-bromo-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(3-phenoxyphenethyl)carbamate (54a)

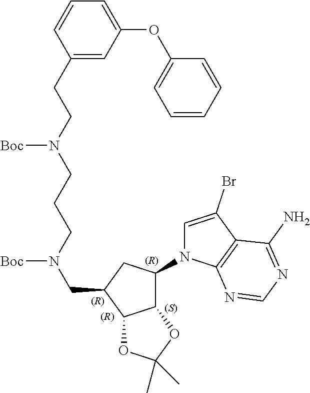

To tert-butyl (3-((((3αR,4R,6R,6αS)-6-(5-bromo-4-chloro-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(3-phenoxyphenethyl)carbamate 53a (40 mg, 0.047 mmol, 1.0 eq) was added NH₃ (7M in MeOH, 5.0 mL) at room temperature and the reaction mixture was stirred at 90° C. overnight. The resulting mixture was allowed to cool to room temperature and was concentrated under vacuum to afford tert-butyl (3-((((3αR,4R,6R,6αS)-6-(4-amino-5-bromo-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(3-phenoxyphenethyl)carbamate 54a (40 mg, crude) as light yellow solid, which was used in the next step without further purification. ¹H NMR (300 MHZ, Chloroform-d) δ 8.25 (s, 1H), 7.42-7.32 (m, 2H), 7.29 (s, 1H), 7.25 (d, J=8.0 Hz, 1H), 7.12 (t, J=7.4 Hz, 1H), 7.07-6.80 (m, 5H), 5.99-5.78 (m, 2H), 5.03-4.81 (m, 2H), 4.59-4.49 (m, 1H), 3.56-3.33 (m, 4H), 3.25-3.18 (m, 5H), 2.88-2.75 (m, 2H), 2.55-2.34 (m, 2H), 1.83-1.72 (m, 2H), 1.55 (s, 3H), 1.45 (s, 18H), 1.28 (s, 3H). LCMS [conditions: Xbridge Shield RP18, 50*3.0 mm, 3.5 μm, Mobile Phase A: Water/0.05% ammonia water, Mobile Phase B: Acetonitrile, 5%-100% B-2.0 min (+), 1.20 mL/min, ES, m/z]: T_{R}=1.59 min; [M+H]⁺: 835, 837.

### ((1R,2S,3R,5R)-3-(4-Amino-5-bromo-7H-pyrrolo[2,3-d]pyrimidin-7-yl)-5-(((3-((3-phenoxyphenethyl)amino)propyl) amino) methyl)cyclopentane-1.7-diol (457)

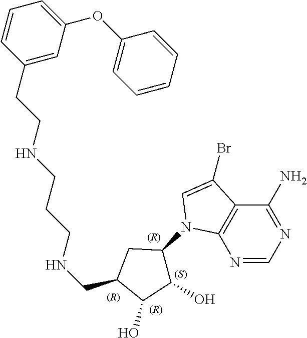

To tert-butyl (3-((((3αR,4R,6R,6αS)-6-(4-amino-5-bromo-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(3-phenoxyphenethyl)carbamate 54a (25 mg, 0.03 mmol, 1.0 eq) was added TFA (1.0 mL) and the reaction mixture was stirred at room temperature for 1 h under nitrogen. The resulting mixture was concentrated under reduced pressure and the crude residue was purified by Prep-HPLC [Column: Sunfire C18 OBD; Mobile phase: A: water (0.01% HCl); B: ACN; Gradient: 32-53% B in 7 min; detector: 220 nm; flow rate: 20 ml/min]: to afford ((1R,2S,3R,5R)-3-(4-amino-5-bromo-7H-pyrrolo[2,3-d]pyrimidin-7-yl)-5-(((3-((3-phenoxyphenethyl) amino)propyl) amino) methyl) cyclopentane-1,2-diol 457 (5.4 mg, 30%) as a white solid. ¹H NMR (400 MHZ, DMSO-d₆) δ 8.08 (s, 1H), 7.56 (s, 1H), 7.38 (t, J=7.9 Hz, 2H), 7.27 (t, J=7.9 Hz, 1H), 7.13 (t, J=7.4 Hz, 1H), 6.98 (t, J=8.3 Hz, 3H), 6.87 (d, J=2.7 Hz, 1H), 6.80 (d, J=8.0 Hz, 1H), 6.67 (s, 2H), 4.87 (q, J=9.0 Hz, 1H), 4.76 (d, J=6.4 Hz, 1H), 4.64-4.53 (m, 1H), 4.20-4.13 (m, 1H), 3.77-3.72 (m, 1H), 2.80-2.59 (m, 7H), 2.35-2.31 (m, 1H), 2.23-2.12 (m, 1H), 2.04-1.94 (m, 1H), 1.61-1.41 (m, 3H). LCMS [Halo C18, 50*3.0 mm, 2.7 μm, Mobile Phase A: Water/0.05% TFA, Mobile Phase B: Acetonitrile, 5%-50%-95% B-3 min, 1.50 mL/min, ES, m/z]: T_{R}=1.27 min; [M+H]⁺: 595, 597.

### Synthesis of (1R,2S,3R,5R)-3-(5-bromo-4-(methylamino)-7H-pyrrolo[2,3-d]pyrimidin-7-yl)-5-(((3-((3-phenoxyphenethyl) amino)propyl) amino) methyl) cyclopentane-1,2-diol (458)

### tert-Butyl-(3-((((3αR,4R,6R,6αS)-6-(5-bromo-4-(methylamino)-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(3-phenoxyphenethyl)carbamate (55a)

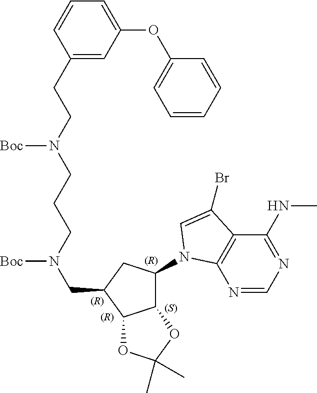

To tert-butyl (3-((((3αR,4R,6R,6αS)-6-(5-bromo-4-chloro-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(3-phenoxyphenethyl)carbamate 53a (140 mg, 0.16 mmol, 1.0 eq) was added CH₃NH₂ (30% in MeOH, 5.0 mL) at room temperature and the reaction mixture was stirred for 2 h at room temperature. The resulting mixture was concentrated under vacuum to afford tert-butyl-(3-((((3αR,4R,6R,6αS)-6-(5-bromo-4-(methylamino)-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(3-phenoxyphenethyl)carbamate 55a (110 mg, 79%) as a light yellow solid. LCMS [HPH-C18, 50*3.0 mm, 2.7 μm, Mobile Phase A: Water/0.05% ammonia water, Mobile Phase B: Acetonitrile, 5%-100% B-2.5 min, 1.50 mL/min, ES, m/z]: T_{R}=1.92 min; [M+H]⁺: 849, 851.

### (1R,2S,3R,5R)-3-(5-Bromo-4-(methylamino)-7H-pyrrolo[2,3-d]pyrimidin-7-yl)-5-(((3-((3-phenoxyphenethyl) amino)propyl) amino) methyl) cyclopentane-1,2-diol (458)

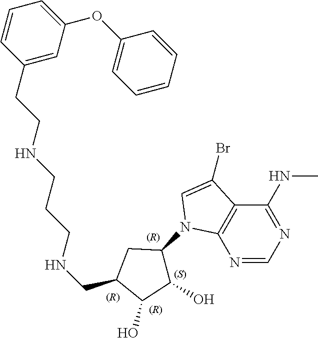

To tert-butyl-(3-((((3αR,4R,6R,6αS)-6-(5-bromo-4-(methylamino)-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(3-phenoxyphenethyl)carbamate 55a (50 mg, 0.059 mmol, 1.0 eq) was added TFA (1.0 mL) and the reaction mixture was stirred at room temperature for 1 h. The resulting mixture was concentrated under vacuum and the crude residue was purified by prep-HPLC [Column: Sunfire C18 OBD; Mobile phase: A: water (0.05% NH₃.H₂O); B: ACN; Gradient: 32-53% B in 7 min; detector: 220 nm; flow rate; 20 mL/min]: to afford (1R,2S,3R,5R)-3-(5-bromo-4-(methylamino)-7H-pyrrolo[2,3-d]pyrimidin-7-yl)-5-(((3-((3-phenoxyphenethyl) amino)propyl) amino) methyl) cyclopentane-1,2-diol 458 (14.3 mg, 40%) as a white solid. ¹H NMR (400 MHZ, DMSO-d₆) δ 8.17 (s, 1H), 7.54 (s, 1H), 7.45-7.32 (m, 2H), 7.27 (s, 1H), 7.18-7.08 (m, 1H), 7.05-6.93 (m, 3H), 6.90-6.75 (m, 2H), 6.55 (s, 1H), 4.98-4.66 (m, 2H), 4.17 (s, 1H), 3.75 (s, 1H), 3.05-2.91 (m, 3H), 2.79-2.63 (m, 4H), 2.25-1.93 (m, 2H), 1.62-1.41 (m, 3H). LCMS [ZORBAX SB-Aq, 50*4.6 mm, 1.8 μm, Mobile Phase A: Water/0.05% TFA, Mobile Phase B: Acetonitrile, 5%-60%-95% B-5 min, 1.50 mL/min, ES, m/z]: T_{R}=2.06 min; [M+H]⁺: 609, 611. Chiral-SFC [Column Name: SB 100×4.6 mm 3.0 μm, Solvent: B: MeOH (20 mM NH₃), Start Conc. of Pump B: 30.0%; Oven Temperature: 35° C.; Total Flow: 3.00 mL/min; BPR Pressure: 15.00 MPa; BPR Temperature: 50° C.]: Two diastereomers; T_{R}=1.44 min (major peak), 1.95 min (minor peak); dr >99:1.

### Synthesis of (1R,2S,3R,5R)-3-(4-Amino-5-ethyl-7H-pyrrolo[2,3-d]pyrimidin-7-yl)-5-(((3-((3-phenoxyphenethyl) amino)propyl) amino) methyl) cyclopentane-1,2-diol (459)

### tert-Butyl(3-((((3αR,4R,6R,6αS)-6-(4-amino-5-vinyl-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(3-phenoxyphenethyl)carbamate (107a)

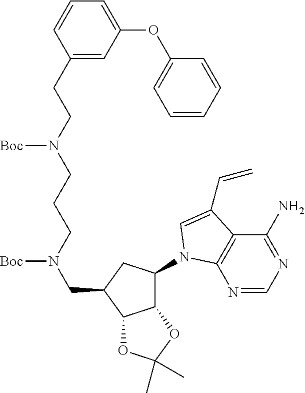 [SMILES: C=Cc1cn([C@@H]2C[C@H](CN(CCCN(CCc3cccc(Oc4ccccc4)c3)C(=O)OC(C)(C)C)C(=O)OC(C)(C)C)[C@H]3OC(C)(C)O[C@H]32)c2ncnc(N)c12]

To a solution of tert-butyl-N-[3-({[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-[2-(3-phenoxyphenyl)ethyl]carbamate 101a (0.13 g, 0.20 mmol, 1.0 eq) and 2-ethenyl-4,4,5,5-tetramethyl-1,3,2-dioxaborolane (36 mg, 0.20 mmol, 1.5 eq) in dioxane (2.0 mL) and H₂O (0.2 mL) were added Na₂CO₃ (49 mg, 0.50 mmol, 3.0 eq) and Pd (dppf) Cl₂ (11 mg, 0.02 mmol, 0.1 eq) and the reaction mixture was stirred at 100° C. for 2 h, under nitrogen. The resulting mixture was concentrated under reduced pressure and the crude residue was purified by silica gel column chromatography (95:5 DCM: MeOH) to afford tert-butyl-N-[3-({[(3αR,4R,6R,6αS)-6-{4-amino-5-ethenylpyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-[2-(3-phenoxyphenyl)ethyl]carbamate 102a (91 mg, 74%) as a yellow solid. LCMS [conditions: XBridge C18, 50*3.0 mm, 3.5 μm, Mobile Phase A: Water/5 mM NH₄HCO₃, Mobile Phase B: Methanol, 10%-95% B-2.0 min (+), 1.20 mL/min, ES, m/z]: T_{R}=1.72 min; [M+H]⁺: 783.

### tert-Butyl-(3-((((3αR,4R,6R,6αS)-6-(4-amino-5-ethyl-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(3-phenoxyphenethyl)carbamate (103a)

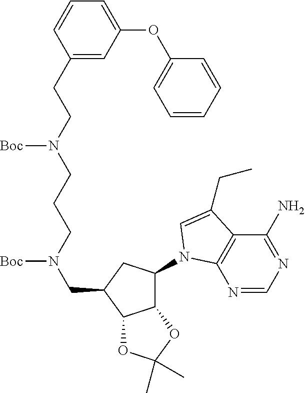 [SMILES: CCc1cn([C@@H]2C[C@H](CN(CCCN(CCc3cccc(Oc4ccccc4)c3)C(=O)OC(C)(C)C)C(=O)OC(C)(C)C)[C@H]3OC(C)(C)O[C@H]32)c2ncnc(N)c12]

To a solution of tert-butyl-N-[3-({[(3αR,4R,6R,6αS)-6-{4-amino-5-ethenylpyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-[2-(3-phenoxyphenyl)ethyl]carbamate 102a (52 mg, 0.07 mmol, 1.0 eq) in 1.0 mL MeOH was added Pd/C(10%, 4 mg) in a pressure tank and the reaction mixture was hydrogenated at room temperature under 5 psi of hydrogen pressure for 1 h. The resulting mixture was filtered through a Celite pad and concentrated under reduced pressure to afford tert-butyl-N-[3-({[(3αR,4R,6R,6αS)-6-{4-amino-5-ethylpyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-[2-(3-phenoxyphenyl)ethyl]carbamate 103a (35 mg, 67%) as a yellow solid. LCMS [conditions: Xbridge Shield RP18, 50*3.0 mm, 3.5 μm, Mobile Phase A: Water/0.05% ammonia water, Mobile Phase B: Acetonitrile, 5%-100% B-2.0 min (+), 1.20 mL/min, ES, m/z]: T_{R}=1.52 min; [M+H]⁺: 785.

### (1R,2S,3R,5R)-3-(4-Amino-5-ethyl-7H-pyrrolo[2,3-d]pyrimidin-7-yl)-5-(((3-((3-phenoxyphenethyl) amino)propyl) amino) methyl) cyclopentane-1,2-diol (459)

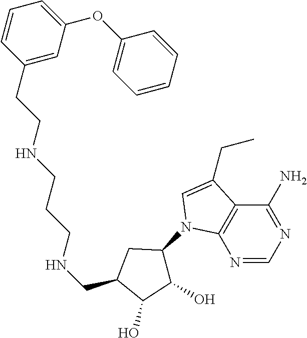 [SMILES: CCc1cn([C@@H]2C[C@H](CNCCCNCCc3cccc(Oc4ccccc4)c3)[C@@H](O)[C@H]2O)c2ncnc(N)c12]

To tert-butyl-N-[3-({[(3αR,4R,6R,6αS)-6-{4-amino-5-ethylpyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-[2-(3-phenoxyphenyl)ethyl]carbamate 103a (35 mg, 0.05 mmol, 1.0 eq) was added TFA (2.0 mL) at room temperature and the reaction mixture was stirred for 2 h at room temperature. The resulting mixture was concentrated under reduced pressure and the residue was basified to pH 10 with NH₃.H₂O. The crude product was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 23% B to 55% B in 7 min, 55% B; Wave Length: 220 nm]: to afford (1R,2S,3R,5R)-3-{4-amino-5-ethylpyrrolo[2,3-d]pyrimidin-7-yl}-5-{[(3-{[2-(3-phenoxyphenyl)ethyl]amino}propyl) amino]methyl}cyclopentane-1,2-diol 459 (12 mg, 49%) as an off-white solid. ¹H NMR (400 MHZ, Chloroform-d) 88.23 (s, 1H), 7.39-7.32 (m, 2H), 7.31-7.26 (m, 1H), 7.16-7.07 (m, 1H), 7.05-6.95 (m, 3H), 6.93-6.84 (m, 2H), 6.73 (s, 1H), 5.18-5.13 (m, 2H), 4.73-4.62 (m, 1H), 4.24-4.16 (m, 1H), 4.06-3.98 (m, 1H), 2.99-2.90 (m, 2H), 2.88-2.79 (m, 10H), 2.53-2.42 (m, 1H), 2.29 (s, 2H), 1.83-1.72 (m, 3H), 1.34 (t, J=7.4 Hz, 3H). LCMS [conditions ZORBAX SB-Aq, 50*4.6 mm, 1.8 μm, Mobile Phase A: Water/0.05% TFA, Mobile Phase B: Acetonitrile, 5%-95% B-3 min, 1.50 mL/min, ES, m/z]: T_{R}=1.29 min; [M+H]⁺: 545. Chiral-SFC [Column: Lux-4 100×4.6 mm 3.0 μm, Solvent B: MeOH (20 mM NH₃), Oven Temperature: 35° C., Total Flow: 3.00 mL/min. Gradient 50% solvent B for 4 mins]: T_{R}=2.50 min, single peak.

### Synthesis of (1R,2S,3R,5R)-3-(4-Amino-5-cyclopropyl-7H-pyrrolo[2,3-d]pyrimidin-7-yl)-5-(((3-((3-phenoxyphenethyl) amino)propyl) amino) methyl) cyclopentane-1,2-diol (460)

### tert-Butyl-(3-((((3αR,4R,6R,6αS)-6-(4-amino-5-cyclopropyl-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(3-phenoxyphenethyl)carbamate (104a)

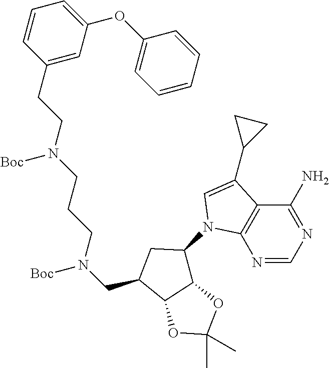 [SMILES: CC(C)(C)OC(=O)N(CCCN(C[C@H]1C[C@@H](n2cc(C3CC3)c3c(N)ncnc32)[C@@H]2OC(C)(C)O[C@H]12)C(=O)OC(C)(C)C)CCc1cccc(Oc2ccccc2)c1]

To a solution of tert-butyl-N-[3-({[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-[2-(3-phenoxyphenyl)ethyl]carbamate 101a (80 mg, 0.10 mmol, 1.0 eq) and cyclopropylboronic acid (16 mg, 0.20 mmol, 2.0 eq) in toluene (2.0 mL) and H₂O (0.10 mL) were added K₃PO₄ (61 mg, 0.30 mmol, 3 eq), Pd(AcO)₂ (2.0 mg, 0.01 mmol, 0.1 eq) and PCy₃ (5.0 mg, 0.02 mmol, 0.2 eq) and the reaction mixture was stirred at 100° C. for 2 h, under nitrogen. The resulting mixture was concentrated under reduced pressure and the crude residue was purified by silica gel column chromatography (90:10 DCM: MeOH) to afford tert-butyl-N-[3-({[(3αR,4R,6R,6αS)-6-{4-amino-5-cyclopropylpyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-[2-(3-phenoxyphenyl)ethyl]carbamate 104a (36 mg, 47%) as a yellow solid. LCMS [conditions: Xbridge Shield RP18, 50*3.0 mm, 3.5 μm, Mobile Phase A: Water/0.05% ammonia water, Mobile Phase B: Acetonitrile, 5%-100% B-2.0 min (+), 1.20 mL/min, ES, m/z]: T_{R}=1.57 min; [M+H]⁺: 797.

### (1R,2S,3R,5R)-3-(4-Amino-5-cyclopropyl-7H-pyrrolo[2,3-d]pyrimidin-7-yl)-5-(((3-((3-phenoxyphenethyl) amino)propyl) aminomethyl) cvrlonentane-12-diol (460)

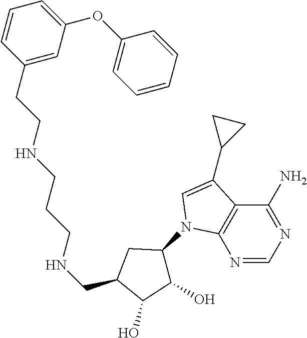 [SMILES: Nc1ncnc2c1c(C1CC1)cn2[C@@H]1C[C@H](CNCCCNCCc2cccc(Oc3ccccc3)c2)[C@@H](O)[C@H]1O]

To tert-butyl-N-[3-({[(3αR,4R,6R,6αS)-6-{4-amino-5-cyclopropylpyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-[2-(3-phenoxyphenyl)ethyl]carbamate 104a (36 mg, 0.05 mmol, 1.0 eq) was added TFA (2.0 mL) at room temperature and the reaction mixture was stirred for 2 h at room temperature. The resulting mixture was concentrated under reduced pressure and basified to pH 10 with NH₃.H₂O. The crude product was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 25% B to 60% B in 7 min, 60% B; Wave Length: 220 nm]: to afford (1R,2S,3R,5R)-3-{4-amino-5-cyclopropylpyrrolo[2,3-d]pyrimidin-7-yl}-5-{[(3-{[2-(3-phenoxyphenyl)ethyl]amino}propyl) amino]methyl}cyclopentane-1,2-diol 460 (8.1 mg, 32%) as an off-white solid. ¹H NMR (400 MHZ, Chloroform-d) 88.20 (s, 1H), 7.39-7.30 (m, 2H), 7.28-7.22 (m, 1H), 7.15-7.07 (m, 1H), 7.05-6.99 (m, 2H), 6.98-6.93 (m, 1H), 6.92-6.83 (m, 2H), 6.69 (s, 1H), 5.54 (s, 2H), 4.72-4.61 (m, 1H), 4.22-4.14 (m, 1H), 4.05-3.98 (m, 1H), 2.93-2.85 (m, 2H), 2.84-2.69 (m, 8H), 2.50-2.39 (m, 1H), 2.34-2.26 (m, 1H), 1.97-1.86 (m, 1H), 1.79-1.64 (m, 3H), 0.95-0.88 (m, 2H), 0.72-0.64 (m, 2H). LCMS [conditions ZORBAX SB-Aq, 50*4.6 mm, 1.8 μm, Mobile Phase A: Water/0.05% TFA, Mobile Phase B: Acetonitrile, 5%-50%-95% B-5 min, 1.50 mL/min, ES, m/z]: T_{R}=2.43 min; [M+H]⁺: 557. Chiral-SFC [Column: Lux-4 100×4.6 mm 3.0 μm, Solvent B: MeOH (20 mM NH₃), Oven Temperature: 35° C., Total Flow: 3.00 mL/min. Gradient 50% solvent B for 4 mins]T_{R}=2.11 min, single peak.

### Synthesis of (1R,2S,3R,5R)-3-(4-Amino-5-phenyl-7H-pyrrolo[2,3-d]pyrimidin-7-yl)-5-(((3-((3-phenoxyphenethyl) amino)propyl) amino) methyl) cyclopentane-1,2-diol (461)

### tert-Butyl-(3-((((3αR,4R,6R,6αS)-6-(4-amino-5-bromo-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(3-phenoxyphenethyl)carbamate (105a)

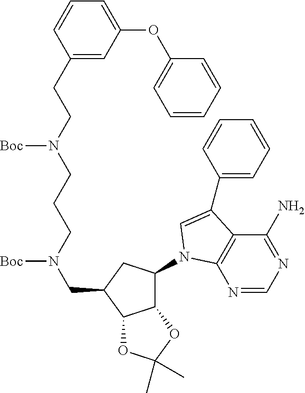 [SMILES: CC(C)(C)OC(=O)N(CCCN(C[C@H]1C[C@@H](n2cc(-c3ccccc3)c3c(N)ncnc32)[C@@H]2OC(C)(C)O[C@H]12)C(=O)OC(C)(C)C)CCc1cccc(Oc2ccccc2)c1]

To a solution of tert-butyl-N-[3-({[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-[2-(3-phenoxyphenyl)ethyl]carbamate 101a (0.10 g, 0.12 mmol, 1.0 eq) and phenyl boronic acid (17 mg, 0.14 mmol, 1.2 eq) in dioxane (2.0 mL) and H₂O (0.2 mL) were added Na₂CO₃ (38 mg, 0.36 mmol, 3.0 eq) and Pd (dppf) Cl₂ (9 mg, 0.01 mmol, 0.1 eq) and the reaction mixture was stirred at 100° C. for 2 h under nitrogen. The resulting mixture was concentrated under reduced pressure and the crude residue was purified by silica gel column chromatography (90:10 DCM: MeOH) to afford tert-butyl-N-[3-({[(3αR,4R,6R,6αS)-6-{4-amino-5-phenylpyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-[2-(3-phenoxyphenyl)ethyl]carbamate 105a (45 mg, 45%) as a yellow solid. LCMS [conditions: Xbridge Shield RP18, 50*3.0 mm, 3.5 μm, Mobile Phase A: Water/5 mM NH₄HCO₃, Mobile Phase B: Methanol, 10%-95% B-2.0 min (+), 1.20 mL/min, ES, m/z]: T_{R}=1.72 min; [M+H]⁺: 833.

### (1R,2S,3R,5R)-3-(4-Amino-5-phenyl-7H-pyrrolo[2,3-d]pyrimidin-7-yl)-5-(((3-((3-phenoxyphemethyl) amino)propyl) aminalmathullevaInnantann 1) dial/1611

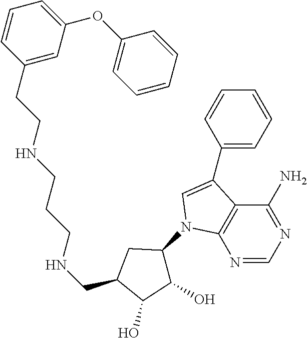 [SMILES: Nc1ncnc2c1c(-c1ccccc1)cn2[C@@H]1C[C@H](CNCCCNCCc2cccc(Oc3ccccc3)c2)[C@@H](O)[C@H]1O]

To tert-butyl-N-[3-({[(3αR,4R,6R,6αS)-6-{4-amino-5-phenylpyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-[2-(3-phenoxyphenyl)ethyl]carbamate 105a (35 mg, 0.04 mmol, 1.0 eq) was added TFA (2.0 mL) and the reaction mixture was stirred for 2 h at room temperature. The reslting mixture was concentrated under reduced pressure and basified to pH 10 with NH₃.H₂O. The crude product was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 ml/min; Gradient: 23% B to 55% B in 7 min, 55% B; Wave Length: 220 nm]: to afford (1R,2S,3R,5R)-3-{4-amino-5-phenylpyrrolo[2,3-d]pyrimidin-7-yl}-5-{[(3-{[2-(3-phenoxyphenyl)ethyl]amino}propyl) amino]methyl}cyclopentane-1,2-diol 461 (7.7 mg, 31%) as an off-white solid. ¹H NMR (400 MHZ, Chloroform-d) 81H NMR (300 MHZ, DMSO-d₆) δ 8.31 (s, 1H), 7.52-7.43 (m, 4H), 7.42-7.29 (m, 3H), 7.28-7.22 (m, 1H), 7.15-7.07 (m, 1H), 7.06-6.98 (m, 3H), 6.97-6.93 (m, 1H), 6.92-6.89 (m, 1H), 6.88-6.83 (m, 1H), 5.16 (s, 2H), 4.86-4.75 (m, 1H), 4.32-4.24 (m, 1H), 4.11-4.03 (m, 1H), 2.94-2.86 (m, 2H), 2.85-2.77 (m, 4H), 2.76-2.70 (m, 4H), 2.57-2.46 (m, 1H), 2.33 (s, 1H), 1.89-1.76 (m, 1H), 1.75-1.67 (m, 2H). LCMS [conditions ZORBAX SB-Aq, 50*4.6 mm, 1.8 μm, Mobile Phase A: Water/0.05% TFA, Mobile Phase B: Acetonitrile, 5%-50%-95% B-5 min, 1.50 mL/min, ES, m/z]: T_{R}=2.18 min; [M+H]⁺: 593. Chiral-SFC [Column: Lux-4 100×4.6 mm 3.0 μm, Solvent B: MeOH (20 mM NH₃), Oven Temperature: 35° C., Total Flow: 3.00 mL/min. Gradient 50% solvent B for 4 mins]: T_{R}=3.04 min, single peak.

### Synthesis of (1R,2S,3R,5R)-3-{4-Amino-5-ethenylpyrrolo[2,3-d]pyrimidin-7-yl}-5-{[(3-{[2-(3-phenoxyphenyl)ethyl]amino}propyl) amino]methyl}cyclopentane-1,2-diol (462)

### (1R,2S,3R,5R)-3-{4-Amino-5-ethenylpyrrolo[2,3-d]pyrimidin-7-yl]-5-{[(3-{[2-(3-phenoxyphenyl)ethyl]amino}propyl) aminolmethyllcyclopentane-1.2-diol (462)

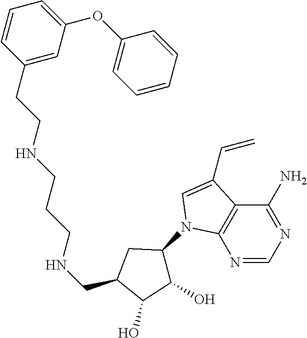 [SMILES: C=Cc1cn([C@@H]2C[C@H](CNCCCNCCc3cccc(Oc4ccccc4)c3)[C@@H](O)[C@H]2O)c2ncnc(N)c12]

To (1R,2S,3R,5R)-3-{4-amino-5-bromopyrrolo[2,3-d]pyrimidin-7-yl}-5-{[(3-{[2-(3-phenoxyphenyl)ethyl]amino}propyl) amino]methyl}cyclopentane-1,2-diol (0.08 g, 0.10 mmol, 1.0 eq), 2-ethenyl-4,4,5,5-tetramethyl-1,3,2-dioxaborolane (0.03 g, 0.20 mmol, 1.5 eq), and Na₂CO₃ (0.04 g, 0.40 mmol, 3.0 eq) in dioxane (1.5 mL) and H₂O (0.2 mL) was added Pd (dppf) Cl₂ (0.01 g, 0.01 mmol, 0.1 eq) at room temperature and the reaction mixture was stirred at 100° C. for 3 h, under N₂. The reaction mixture was filtered and the filtrate was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 ml/min; Gradient: 24% B to 49% B in 7 min, 49% B; Wave Length: 220 nm]: to afford (1R,2S,3R,5R)-3-{4-amino-5-ethenylpyrrolo[2,3-d]pyrimidin-7-yl}-5-{[(3-{[2-(3-phenoxyphenyl)ethyl]amino}propyl) amino]methyl}cyclopentane-1,2-diol 462 (15 mg, 20%) as an off-white solid. ¹H NMR (300 MHZ, DMSO-d₆) δ8.03 (s, 1H), 7.62-7.57 (m, 1H), 7.43-7.35 (m, 2H), 7.34-7.23 (m, 1H), 7.19-7.04 (m, 2H), 7.02-6.94 (m, 3H), 6.91-6.86 (m, 1H), 6.85-6.79 (m, 1H), 6.64-6.58 (m, 2H), 5.55 (d, J=17.5 Hz, 1H), 5.08 (d, J=11.0 Hz, 1H), 4.93-4.79 (m, 1H), 4.24-4.15 (m, 1H), 3.85-3.73 (m, 1H), 2.88-2.59 (m, 10H), 2.31-2.00 (m, 2H), 1.73-1.39 (m, 3H). LCMS [conditions Halo C18, 50*3.0 mm, 2.7 μm, Mobile Phase A: Water/0.05% TFA, Mobile Phase B: Acetonitrile, 5%-95% B-3 min, 1.50 L/min, ES, m/z), 2 min, ES, m/z]: T_{R}=1.30 min; [M+H]⁺: 543 Chiral-SFC [Column: IG 100×4.6 mm 3.0 μm, Solvent B: MeOH (20 mM NH₃), Oven Temperature: 35° C., Total Flow: 3.00 mL/min. Gradient 50% solvent B for 4 mins]: T_{R}=1.97 min, single peak.

### Synthesis of (1R,2S,3R,5R)-3-(4-amino-5-bromo-7H-pyrrolo[2,3-d]pyrimidin-7-yl)-5-(((3-(phenethylamino)propyl) amino) methyl) cyclopentane-1,2-diol (463)

### Synthesis of tert-Butyl-N-(3-aminopropyl)-N-(2-phenylethyl)carbamate (110a) 3-(Phenethylamino)propan-1-ol (56a)

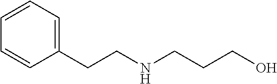 [SMILES: OCCCNCCc1ccccc1]

A mixture of 2-bromoethyl benzene (5.00 g, 27.0 mmol, 1.0 eq) and propanolamine (12.2 g, 162 mmol, 6.0 eq) in ethanol (30 mL) was heated at 80° C. for 3 h. The reaction mixture was then concentrated under vacuum, diluted with DCM and washed with water. The aqueous layer was extracted with DCM and the combined organic layers were washed with brine, dried over sodium sulfate, filtered, and concentrated to afford 3-(phenethylamino)propan-1-ol 56a (4.50 g, 85%) as a light yellow oil. ¹H NMR (300 MHZ, DMSO-d₆) δ 7.35-7.24 (m, 2H), 7.23-7.12 (m, 3H), 3.46 (t, J=6.3 Hz, 2H), 2.70 (q, J=5.0, 4.0 Hz, 4H), 2.60 (t, J=6.8 Hz, 2H), 1.55 (p, J=6.6 Hz, 2H).

### tert-Butyl(3-hydroxypropyl)(phenethyl)carbamate (57a)

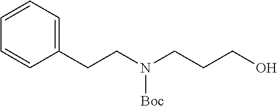 [SMILES: CC(C)(C)OC(=O)N(CCCO)CCc1ccccc1]

To a stirred mixture of 3-(phenethylamino)propan-1-ol 56 (4.50 g, 25.1 mmol, 1.0 eq) and Et₃N (5.10 g, 50.3 mmol, 2.0 eq) in DCM (80 mL) was added di-tert-butyl dicarbonate (6.60 g, 30.2 mmol, 1.2 eq) in several portions at 0° C. and the reaction mixture was stirred for 3 h at room temperature. The reaction was quenched with water (100 mL) and then extracted with DCM (60 mL). The organic layer were dried over anhydrous Na₂SO₄, filtered and the filtrate was concentrated under reduced pressure. The crude residue was purified by silica gel column chromatography (6:1 Petroleum ether/AcOEt) to afford tert-butyl (3-hydroxypropyl)(phenethyl)carbamate 57a (5.00 g, 71%) as a colorless oil. ¹H NMR (300 MHZ, DMSO-d₆) δ 7.35-7.25 (m, 2H), 7.24-7.15 (m, 3H), 4.41 (t, J=5.1 Hz, 1H), 3.44-3.28 (m, 4H), 3.16 (t, J=7.2 Hz, 2H), 2.76 (dd, J=8.6, 6.4 Hz, 2H), 1.68-1.53 (m, 2H), 1.36 (s, 9H). tert-Butyl(3-(1,3-dioxoisoindolin-2-yl)propyl)(phenethyl)carbamate (140a)

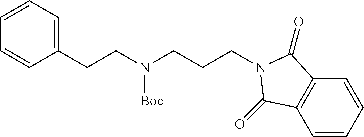 [SMILES: CC(C)(C)OC(=O)N(CCCN1C(=O)c2ccccc2C1=O)CCc1ccccc1]

To a stirred mixture of tert-butyl (3-hydroxypropyl)(phenethyl)carbamate 57a (5.00 g, 17.9 mmol, 1.0 eq), phthalimide (5.30 g, 35.8 mmol, 2.0 eq) and PPh₃ (9.40 g, 35.8 mmol, 2.0 eq) in THF (50 mL) was added DIAD (7.20 g, 35.8 mmol, 2.0 eq), dropwise, at 0° C. and the reaction mixture was stirred for 2 h, at 0° C., under nitrogen. The resulting mixture was quenched by the addition of water (20 mL) at room temperature and extracted with EtOAc (3×150 mL). The combined organic layers were washed with brine, dried over anhydrous Na₂SO₄ and filtered. The filtrate was concentrated under reduced pressure and the crude residue was purified by silica gel column chromatography (80:20 Petroleum ether: AcOEt) to afford tert-butyl (3-(1,3-dioxoisoindolin-2-yl)propyl)(phenethyl)carbamate 140a (5.50 g, 75%) as a white solid.

### tert-Butyl(3-aminopropyl)(phenethyl)carbamate (110a)

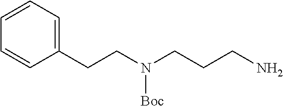 [SMILES: CC(C)(C)OC(=O)N(CCCN)CCc1ccccc1]

To a solution of tert-butyl (3-(1,3-dioxoisoindolin-2-yl)propyl)(phenethyl)carbamate 140a (5.50 g, 13.5 mmol, 1.0 eq) in EtOH (50 mL) was added NH₂NH₂·H₂O (3.30 g, 67.4 mmol, 5.0 eq), dropwise, at room temperature, under nitrogen and the reaction mixture was stirred at 80° C. for 3 h. The resulting mixture was allowed to cool to room temperature and concentrated in vacuo and the residue was partitioned between water (20 mL) and DCM (80 mL). The organic layer was washed with brine, dried over anhydrous Na₂SO₄, filtered and concentrated in vacuum to afford tert-butyl (3-aminopropyl)(phenethyl)carbamate 110a (3.20 g, 85%) as a brown oil, which was used in the next step without further purification.

### tert-Butyl(3-((tert-butoxycarbonyl)(((3αR,4R,6R,6αS)-6-(4-chloro-7H-pyrrolo[2,3-d]pyrimidin-7-yl)-2,2-dimethyltetrahydro-4H-cyclopenta[d][1,3]dioxol-4-yl)methyl) amino)propyl)(phenethyl)carbamate (107a)

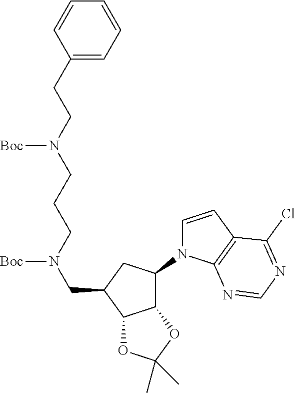 [SMILES: CC(C)(C)OC(=O)N(CCCN(C[C@H]1C[C@@H](n2ccc3c(Cl)ncnc32)[C@@H]2OC(C)(C)O[C@H]12)C(=O)OC(C)(C)C)CCc1ccccc1]

To tert-butyl-N-(3-aminopropyl)-N-(2-phenylethyl)carbamate 110a (1.82 g, 6.50 mmol, 1.2 eq), and NaBH (OAc)₃ (2.31 g, 10.9 mmol, 2.0 eq) in DCM (25.0 mL) was added AcOH (3.27 g, 54.4 mmol, 10.0 eq) at room temperature and the resulting mixture was stirred for 30 min at room temperature. Then (3αR,4S,6R,6αS)-6-{4-chloropyrrolo[2,3-d]pyrimidin-7-yl}-2,2-dimethyl-tetrahydro-3αH-cyclopenta[d][1,3]dioxole-4-carbaldehyde 77a (2.50 g, 5.40 mmol, 1.0 eq) in DCM (10.0 mL) was added and the reaction mixture was stirred for 1 h at room temperature. After this time, Boc₂O (1.78 g, 8.20 mmol, 1.5 eq) and TEA (8.26 g, 81.6 mmol, 15.0 eq) were added and the reaction mixture was stirred for 30 min at room temperature. The resulting mixture was concentrated under reduced pressure and the crude product was purified by prep-HPLC [Column: Xtimate C18 50×250 mm, 10 μm; Mobile Phase A: Water (0.1% NH₃.H₂O), Mobile Phase B: ACN; Flow rate: 90 ml/min; Gradient: 45% B to 75% B in 12 min; Wave Length: 220 nm]: to afford tert-butyl-N-[3-({[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 107a (1.43 g, 38%) as a yellow solid. ¹H NMR (400 MHZ, Chloroform-d) 8 8.65 (s, 1H), 7.38-7.29 (m, 3H), 7.27-7.14 (m, 3H), 6.67 (d, J=3.6 Hz, 1H), 5.11-4.83 (m, 2H), 4.69-4.51 (m, 1H), 3.55-3.31 (m, 4H), 3.29-3.04 (m, 4H), 2.93-2.78 (m, 2H), 2.55-2.36 (m, 2H), 2.27-1.95 (m, 1H), 1.86-1.70 (m, 2H), 1.57 (s, 3H), 1.49-1.42 (m, 18H), 1.29 (s, 3H). LCMS [conditions: XBridge C18, 50*3.0 mm, 3.5 μm, Mobile Phase A: Water/5 mM NH₄HCO₃, Mobile Phase B: Methanol, 10%-95% B-2 min (+), 1.50 mL/min]: T_{R}=2.09 min; [M+H]⁺: 684.

### tert-Butyl-(3-((((3αR,4R,6R,6αS)-6-(5-bromo-4-chloro-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(phenethyl)carbamate (108a)

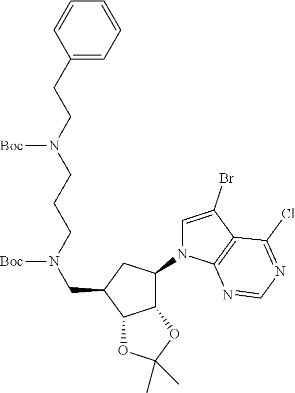 [SMILES: CC(C)(C)OC(=O)N(CCCN(C[C@H]1C[C@@H](n2cc(Br)c3c(Cl)ncnc32)[C@@H]2OC(C)(C)O[C@H]12)C(=O)OC(C)(C)C)CCc1ccccc1]

To tert-butyl-N-[3-({[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 107a (1.20 g, 1.80 mmol, 1.0 eq) was added NBS (0.37 g, 2.10 mmol, 1.2 eq) in DMF (15 mL) at room temperature and the reaction mixture was stirred for 2 h at room temperature. The reaction was quenched by the addition of water (30 mL) at room temperature and the reaction mixture was extracted with AcOEt (3×10 mL). The combined organic extracts were concentrated under reduced pressure and the crude residue was purified by Prep-TLC(95:5 DCM/MeOH) to afford tert-butyl-N-[3-({[(3αR,4R,6R,6αS)-6-{5-bromo-4-chloropyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 108a (1.21 g, 90%) as a yellow solid. ¹H NMR (400 MHZ, Chloroform-d) 88.63 (s, 1H), 7.39-7.30 (m, 2H), 7.26-7.15 (m, 4H), 5.08-4.81 (m, 2H), 4.63-4.49 (m, 1H), 3.55-3.31 (m, 3H), 3.28-3.09 (m, 5H), 2.88-2.79 (m, 2H), 2.53-2.37 (m, 2H), 2.00-1.89 (m, 1H), 1.84-1.71 (m, 2H), 1.55 (s, 3H), 1.50-1.41 (m, 18H), 1.28 (s, 3H). LCMS [conditions: Kinetex XB-C18, 3.0*30 mm, 1.7 μm, Mobile Phase A: Water/0.1% FA, Mobile Phase B: Acetonitrile/0.05% FA, 5%-95% B-2 min (+).lcm, 1.2 mL/min]: T_{R}=1.75: [M+H]⁺: 762, 764.

### tert-Butyl-(3-((((3αR,4R,6R,6αS)-6-(4-amino-5-bromo-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(phenethyl)carbamate (109a)

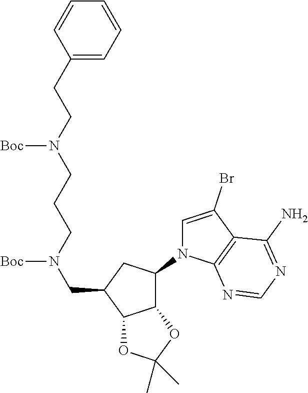 [SMILES: CC(C)(C)OC(=O)N(CCCN(C[C@H]1C[C@@H](n2cc(Br)c3c(N)ncnc32)[C@@H]2OC(C)(C)O[C@H]12)C(=O)OC(C)(C)C)CCc1ccccc1]

To tert-butyl-N-[3-({[(3αR,4R,6R,6αS)-6-{5-bromo-4-chloropyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 108a (1.00 g, 1.30 mmol, 1.0 eq) was added NH₃ (7M in MeOH, 10 mL) at room temperature and the reaction mixture was stirred at 75° C. for 12 h. The resulting mixture was concentrated under reduced pressure and the crude residue was purified by silica gel column chromatography (1:1 Petroleum ether/AcOEt) to afford tert-butyl-N-[3-({[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino) propyl]-N-(2-phenylethyl)carbamate 109 (0.70 g, 72%) as an off-white solid. LCMS [conditions: Kinetex XB-C18, 3.0*30 mm, 1.7 μm, Mobile Phase A: Water/0.1% FA, Mobile Phase B: Acetonitrile/0.05% FA, 5%-95% B-2 min (+).lcm, 1.2 mL/min]: T_{R}=1.42 min; [M+H]⁺: 743, 745. ¹H NMR (400 MHZ, Chloroform-d) δ 8.25 (s, 1H), 7.35-7.30 (m, 2H), 7.26-7.21 (m, 3H), 7.08 (s, 1H), 6.13 (s, 2H), 5.05-4.79 (m, 2H), 4.60-4.48 (m, 1H), 3.49-3.29 (m, 4H), 3.28-3.06 (m, 4H), 2.92-2.79 (m, 2H), 2.53-2.33 (m, 2H), 2.30-2.00 (m, 1H), 1.87-1.70 (m, 2H), 1.55 (s, 3H), 1.47-1.41 (m, 18H), 1.28 (s, 3H).

### (1R,2S,3R,5R)-3-(4-Amino-5-bromo-7H-pyrrolo[2,3-d]pyrimidin-7-yl)-5-(((3-(phenethylamino)propyl) amino) methyl) cyclopentane-1,2-diol (463)

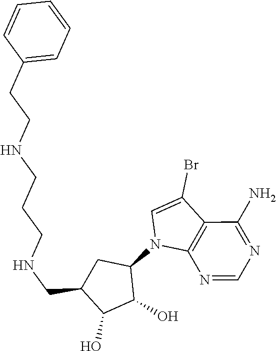 [SMILES: Nc1ncnc2c1c(Br)cn2[C@@H]1C[C@H](CNCCCNCCc2ccccc2)[C@@H](O)[C@H]1O]

To tert-butyl-N-[3-({[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 109a (35 mg, 0.05 mmol, 1.0 eq) was added TFA (1.0 mL, 13.5 mmol, 286 eq) and the reaction mixture was stirred for 0.5 h at 50° C. The resulting mixture was concentrated under reduced pressure and basified to pH >8 with NH₃.H₂O. The crude residue was dissolved in DMF (3.0 mL) and purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 24% B to 49% B in 7 min, 49% B; Wave Length: 220 nm]: to afford (1R,2S,3R,5R)-3-{4-amino-5-bromopyrrolo[2,3-d]pyrimidin-7-yl}-5-[{{3-[(2-phenylethyl) amino]propyl}amino) methyl]cyclopentane-1,2-diol 463 (10.8 mg, 45%) as an off-white solid. ¹H NMR (400 MHZ, DMSO-d₆) δ 8.08 (s, 1H), 7.58 (s, 1H), 7.30-7.22 (m, 2H), 7.21-7.12 (m, 3H), 6.68 (s, 2H), 4.93-4.83 (m, 1H), 4.82-4.77 (m, 1H), 4.21-4.13 (m, 1H), 3.76-3.74 (m, 1H), 2.73-2.56 (m, 10H), 2.22-2.10 (m, 1H), 2.05-2.01 (m, 1H), 1.60-1.41 (m, 3H). LCMS [conditions: ZORBAX SB-Aq, 50*4.6 mm, 1.8 μm, Mobile Phase A: Water/0.05% TFA, Mobile Phase B: Acetonitrile, 5%-95% B-3 min, 1.50 mL/min]: T_{R}=0.99 min; [M+H]⁺: 503, 505. Chiral-SFC [Column: SC 100×4.6 mm 3.0 μm, Solvent B: MeOH (20 mM NH₃), Oven Temperature: 50° C., Total Flow: 3.00 mL/min. Gradient 30% solvent B for 5 mins]: T_{R}=2.93 min, single peak.

### Synthesis of (1R,2S,3R,5R)-3-(4-amino-5-vinyl-7H-pyrrolo[2,3-d]pyrimidin-7-yl)-5-(((3-(phenethylamino)propyl) amino) methyl) cyclopentane-1,2-diol (464)

### (1R,2S,3R,5R)-3-(4-Amino-5-vinyl-7H-pyrrolo[2,3-d]pyrimidin-7-yl)-5-(((3-(phenethylamino)propyl) amino) methyl) cyclopentane-1.2-diol (464)

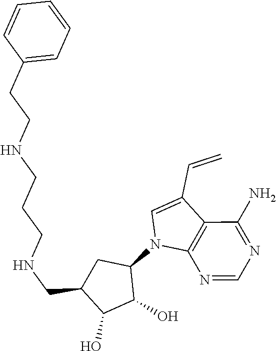 [SMILES: C=Cc1cn([C@@H]2C[C@H](CNCCCNCCc3ccccc3)[C@@H](O)[C@H]2O)c2ncnc(N)c12]

To a stirred mixture of (1R,2S,3R,5R)-3-{4-amino-5-bromopyrrolo[2,3-d]pyrimidin-7-yl}-5-[({3-[(2-phenylethyl) amino]propyl}amino) methyl]cyclopentane-1,2-diol 325 (50 mg, 0.10 mmol, 1.0 eq) and 2-ethenyl-4,4,5,5-tetramethyl-1,3,2-dioxaborolane (21 mg, 0.13 mmol, 1.5 eq) in 1,4-dioxane (1.0 mL) and H₂O (0.1 mL) were added Pd (dtbpf) Cl₂ (2.91 mg, 0.004 mmol, 0.05 eq) and K₃PO₄ (57 mg, 0.30 mmol, 3.0 eq) and the resulting mixture was stirred at 80° C., for 0.5 h, under nitrogen. The resulting mixture was concentrated under reduced pressure and partitioned between AcOEt and water. The aqueous layer was extracted with AcOEt (x2) and the combined organic layers were washed with brine, dried over anhydrous Na₂SO₄ and filtered. The filtrate was concentrated under reduced pressure and the resulting crude residue was dissolved in methanol (3.0 mL) and purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 20% B to 55% B in 7 min, 55% B; Wave Length: 220 nm]: to afford (1R,2S,3R,5R)-3-{4-amino-5-ethenylpyrrolo[2,3-d]pyrimidin-7-yl}-5-[({3-[(2-phenylethyl) amino]propyl}amino) methyl]cyclopentane-1,2-diol 464 (16.3 mg, 40%) as a white solid. ¹H NMR (400 MHZ, DMSO-d₆+D₂O) 88.03 (s, 1H), 7.61 (s, 1H), 7.30-7.23 (m, 2H), 7.22-7.04 (m, 4H), 5.62 (d, J=17.2 Hz, 1H), 5.09 (dd, J=10.8, 1.2 Hz, 1H), 4.87 (q, J=8.4 Hz, 1H), 4.26-4.13 (m, 1H), 3.84-3.72 (m, 1H), 3.25-2.53 (m, 10H), 2.23-2.00 (m, 2H), 1.66-1.41 (m, 3H). LCMS (conditions: ZORBAX SB-Aq, 50*4.6 mm, 1.8 μm, Mobile Phase A: Water/0.05% TFA, Mobile Phase B: Acetonitrile, 5%-95% B-3 min, 1.50 mL/min), T_{R}=0.58 min; [M+H]⁺: 451. Chiral-SFC [Column: IG 100×4.6 mm 3.0 μm, Solvent B: MeOH (20 mM NH₃), Oven Temperature: 35° C., Total Flow: 3.00 mL/min. Gradient 40% solvent B for 4 mins]: T_{R}=1.76 min, single peak.

### Synthesis of (1S,2R,35,5S)-3-(4-Amino-5-ethyl-7H-pyrrolo[2,3-d]pyrimidin-7-yl)-5-(((3-(phenethylamino)propyl) amino) methyl) cyclopentane-1,2-diol (465)

### tert-Butyl-(3-((((3αR,4R,6R,6αS)-6-(4-amino-5-vinyl-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino) propyl)(phenethyl)carbamate (111a)

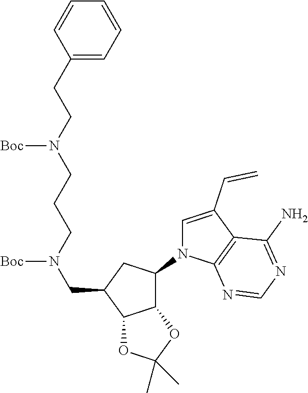 [SMILES: C=Cc1cn([C@@H]2C[C@H](CN(CCCN(CCc3ccccc3)C(=O)OC(C)(C)C)C(=O)OC(C)(C)C)[C@H]3OC(C)(C)O[C@H]32)c2ncnc(N)c12]

To a stirred solution of tert-butyl-N-[3-({[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 108a (70 mg, 0.10 mmol, 1.0 eq) and 2-ethenyl-4,4,5,5-tetramethyl-1,3,2-dioxaborolane (29 mg, 0.20 mmol, 2.0 eq) in 1,4-dioxane (1.0 mL) and H₂O (0.1 mL) were added Pd (dtbpf) Cl₂ (3 mg, 0.005 mmol, 0.05 eq) and K₃PO₄ (60 mg, 0.3 mmol, 3.0 eq) and the reaction mixture was stirred at 80° C. for 0.5 h, under nitrogen. The resulting mixture was concentrated under reduced pressure and partitioned between AcOEt and water. The aqueous layer was extracted with AcOEt (2×) and the combined organic layers were washed with brine, dried over anhydrous Na₂SO₄ and filtered. The filtrate was concentrated under reduced pressure and the crude residue was purified by silica gel column chromatography (50:50 AcOEt: petroleum ether) to afford tert-butyl-N-[3-({[(3αR,4R,6R,6αS)-6-{4-amino-5-ethenylpyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 111a (60 mg, 88%) as a white solid. LCMS [conditions: Kinetex XB-C18, 50*3.0 mm, 2.6 μm, Mobile Phase A: Water/0.05% TFA, Mobile Phase B: Acetonitrile, 2%-95% B-3 min (+).Icm, 1.50 mL/min]: T_{R}=1.71 min; MS [M+H]⁺: 691.

### tert-Butyl-(3-((((3αR,4R,6R,6αS)-6-(4-amino-5-ethyl-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(phenethyl)carbamate (112a)

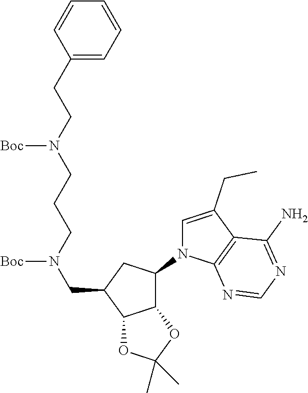 [SMILES: CCc1cn([C@@H]2C[C@H](CN(CCCN(CCc3ccccc3)C(=O)OC(C)(C)C)C(=O)OC(C)(C)C)[C@H]3OC(C)(C)O[C@H]32)c2ncnc(N)c12]

To a solution of tert-butyl-N-[3-({[(3αR,4R,6R,6αS)-6-{4-amino-5-ethenylpyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 111a (60 mg, 0.10 mmol, 1.0 eq) in 2.0 mL MeOH was added Pd/C(10%, 2.0 mg) in a pressure tank and the reaction mixture was hydrogenated at room temperature under 5 psi of hydrogen pressure for 0.5 h. The resultant mixture was filtered through a Celite pad and concentrated under reduced pressure to afford the crude product tert-butyl-N-[3-({[(3αR,4R,6R,6αS)-6-{4-amino-5-ethylpyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 112a (55 mg, 96%) which was used in the next step without further purification. LCMS [conditions: XBridge C18, 50*3.0 mm, 3.5 μm, Mobile Phase A: Water/5 mM NH₄HCO₃, Mobile Phase B: Methanol, 10%-95% B-2 min (+).lcm, 1.00 mL/min]: T_{R}=1.60 min; [M+H]⁺: 693.

### (1R,2S,3R,5R)-3-(4-Amino-5-ethyl-7H-pyrrolo[2,3-d]pyrimidin-7-yl)-5-(((3-(phenethylamino)propyl) amino) methyl) cyclopentane-1.2-diol (465)

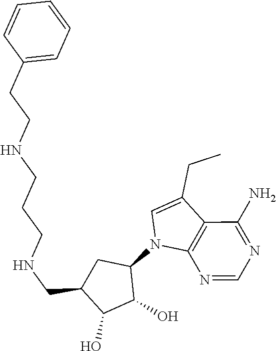 [SMILES: CCc1cn([C@@H]2C[C@H](CNCCCNCCc3ccccc3)[C@@H](O)[C@H]2O)c2ncnc(N)c12]

To tert-butyl-N-[3-({[(3αR,4R,6R,6αS)-6-{4-amino-5-ethylpyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 112a (55 mg, 0.1 mmol, 1.0 eq) was added TFA (1.0 mL) and the resulting mixture was stirred at 50° C. for 0.5 h. The resulting mixture was allowed to cool and was concentrated under reduced pressure and basified to pH >8 with NH₃.H₂O. The crude residue was dissolved in DMF (3.0 mL) and purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 25% B to 50% B in 7 min, 50% B; Wave Length: 220 nm]: to afford (1R,2S,3R,5R)-3-{4-amino-5-ethylpyrrolo[2,3-d]pyrimidin-7-yl}-5-[{{3-[(2-phenylethyl) amino]propyl}amino) methyl]cyclopentane-1,2-diol 465 (18 mg, 48%) as an off-white solid. ¹H NMR (300 MHZ, DMSO-d₆+D₂O) 87.98 (s, 1H), 7.35-7.12 (m, 5H), 7.01 (s, 1H), 4.89-4.74 (m, 1H), 4.19-4.07 (m, 1H), 3.86-3.68 (m, 1H), 3.30-2.82 (m, 6H), 2.79-2.67 (m, 5H), 2.61-2.59 (m, 1H), 2.19-2.04 (m, 2H), 1.68-1.54 (m, 2H), 1.52-1.37 (m, 1H), 1.20 (t, J=7.4 Hz, 3H). LCMS [conditions: ZORBAX SB-Aq, 50*4.6 mm, 1.8 μm, Mobile Phase A: Water/0.05% TFA, Mobile Phase B: Acetonitrile, 5%-95% B-3 min, 1.50 mL/min]: T_{R}=0.98 min; [M+H]⁺: 453. Chiral-SFC [Column: IG 100×4.6 mm 3.0 μm, Solvent B: MeOH (20 mM NH₃), Oven Temperature: 35° C., Total Flow: 3.00 mL/min]: T_{R}=2.04 min; single peak

### Synthesis of (1R,2S,3R,5R)-3-(4-amino-5-phenyl-7H-pyrrolo[2,3-d]pyrimidin-7-yl)-5-(((3-(phenethylamino)propyl) amino) methyl) cyclopentane-1,2-diol (466)

### tert-Butyl-(3-((((3αR,4R,6R,6αS)-6-(4-amino-5-phenyl-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(phenethyl)carbamate (113a)

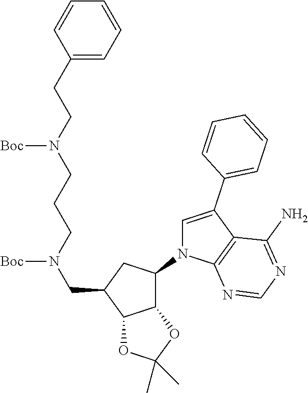 [SMILES: CC(C)(C)OC(=O)N(CCCN(C[C@H]1C[C@@H](n2cc(-c3ccccc3)c3c(N)ncnc32)[C@@H]2OC(C)(C)O[C@H]12)C(=O)OC(C)(C)C)CCc1ccccc1]

To a stirred mixture of tert-butyl-N-[3-({[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 108a (70 mg, 0.1 mmol, 1.0 eq) and phenyl boronic acid (18 mg, 0.15 mmol, 1.5 eq) in 1,4-dioxane (1.0 mL) and H₂O (0.1 mL) were added Pd (dtbpf) Cl₂ (3 mg, 0.005 mmol, 0.05 eq) and K₃PO₄ (60 mg, 0.3 mmol, 3.0 eq) and the reaction mixture was stirred at 80° C. for 1 h under nitrogen. The resulting mixture was concentrated under reduced pressure and partitioned between AcOEt and water. The aqueous layer was extracted with AcOEt (2×) and the combined organic layers were washed with brine, dried over anhydrous Na₂SO₄ and filtered. The filtrate was concentrated and the crude residue was purified by silica gel column chromatography (50:50 THF: petroleum ether) to afford tert-butyl-N-[3-({[(3αR,4R,6R,6αS)-6-{4-amino-5-phenylpyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 113a (60 mg, 86%) as a yellow solid. LCMS [conditions: XBridge C18, 50*3.0 mm, 3.5 μm, Mobile Phase A: Water/5 mM NH₄HCO₃, Mobile Phase B: Methanol, 10%-95% B-2 min (+).lcm, 1.00 mL/min]: T_{R}=1.66 min; [M+H]⁺: 741.

### (1R,2S,3R,5R)-3-(4-Amino-5-phenyl-7H-pyrrolo[2,3-d]pyrimidin-7-yl)-5-(((3-(phenethylamino)propyl) amino) methyl) cyclopentane-1,2-diol (466)

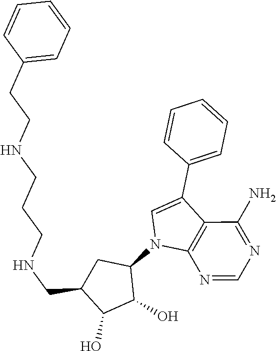 [SMILES: Nc1ncnc2c1c(-c1ccccc1)cn2[C@@H]1C[C@H](CNCCCNCCc2ccccc2)[C@@H](O)[C@H]1O]

To tert-butyl-N-[3-({[(3αR,4R,6R,6αS)-6-{4-amino-5-phenylpyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 113a (60 mg, 0.05 mmol, 1.0 eq) was aded TFA (1.0 mL) and the reaction mixture was stirred at 50° C. for 1 h. The resulting mixture was concentrated under vacuum and was basified to pH >8 with ammonium hydroxide. The crude residue was dissolved in DMF (3.0 mL) and purified by Prep-HPLC [Column: YMC-Actus Triart C₁₈ ExRS, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 24% B to 49% B in 7 min, 49% B; Wave Length: 220 nm]: to afford (1R,2S,3R,5R)-3-{4-amino-5-phenylpyrrolo[2,3-d]pyrimidin-7-yl}-5-[({3-[(2-phenylethyl) amino]propyl}amino) methyl]cyclopentane-1,2-diol 466 (12.3 mg, 50%) as a white solid. ¹H NMR (400 MHZ, Chloroform-d) 88.27 (s, 1H), 7.52-7.44 (m, 4H), 7.37 (br s, 1H), 7.32-7.26 (m, 2H), 7.22-7.17 (m, 3H), 6.98 (s, 1H), 5.17 (br s, 2H), 4.86-4.73 (m, 1H), 4.30-4.22 (m, 1H), 4.03 (br s, 1H), 2.93-2.88 (m, 2H), 2.85-2.70 (m, 8H), 2.50-2.42 (m, 1H), 2.34-2.22 (m, 1H), 1.82-1.68 (m, 3H). LCMS [conditions: ZORBAX SB-Aq, 50*4.6 mm, 1.8 μm, Mobile Phase A: Water/0.05% TFA, Mobile Phase B: Acetonitrile, 5%-95% B-3 min, 1.50 mL/min]: T_{R}=1.08 min; [M+H]⁺: 501. Chiral-SFC [Column: SC 100×4.6 mm 3.0 μm, Solvent B: MeOH (20 mM NH₃), Oven Temperature: 35° C., Total Flow: 3.00 mL/min. Gradient 30% solvent B for 4 mins]: T_{R}=2.90 min, single peak.

### Synthesis of (1R,2S,3R,5R)-3-(4-amino-5-methyl-7H-pyrrolo[2,3-d]pyrimidin-7-yl)-5-(((3-(phenethylamino)propyl) amino) methyl) cyclopentane-1,2-diol (467)

### tert-Butyl-(3-((((3αR,4R,6R,6αS)-6-(4-amino-5-methyl-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(phenethyl)carbamate (177a)

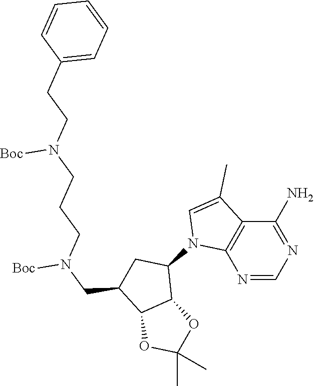 [SMILES: Cc1cn([C@@H]2C[C@H](CN(CCCN(CCc3ccccc3)C(=O)OC(C)(C)C)C(=O)OC(C)(C)C)[C@H]3OC(C)(C)O[C@H]32)c2ncnc(N)c12]

To a solution of tert-butyl-N-[3-({[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 108a (80 mg, 0.10 mmol, 1.0 eq) and 2,4,6-trimethyl-1,3,5,2,4,6-trioxatriborinane (27 mg, 0.20 mmol, 2.0 eq) in dioxane (2.0 mL) and H₂O (0.2 mL) was added Na₂CO₃ (34 mg, 0.30 mmol, 3.0 eq) and Pd (dppf) Cl₂ (16 mg, 0.02 mmol, 0.2 eq) and the reaction mixture was stirred at 100° C., for 3 h, under nitrogen. The resulting mixture was concentrated under reduced pressure and the crude residue was purified by silica gel column chromatography (90:10 CH₂Cl₂: MeOH) to afford tert-butyl-N-[3-({[(3αR,4R,6R,6αS)-6-{4-amino-5-methylpyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 122a (27 mg, 37%) as a yellow solid. LCMS [conditions: Xbridge Shield RP18, 50*3.0 mm, 3.5 μm; Mobile Phase A: Water/0.05% Ammonia, Mobile Phase B: Acetonitrile, 5%-100% B-2 min (+), 1.50 mL/min, ES, m/z]: T_{R}=1.27 min; [M+H]⁺: 679.

### (1R,2S,3R,5R)-3-(4-Amino-5-methyl-7H-pyrrolo[2,3-d]pyrimidin-7-yl)-5-(((3-(phenethylamino)propyl) amino) methyl) cyclopentane-1,2-diol (467)

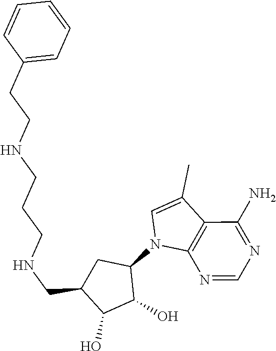 [SMILES: Cc1cn([C@@H]2C[C@H](CNCCCNCCc3ccccc3)[C@@H](O)[C@H]2O)c2ncnc(N)c12]

To tert-butyl-N-[3-({[(3αR,4R,6R,6αS)-6-{4-amino-5-methylpyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 122a (27 mg, 0.04 mmol, 1.0 eq) was added TFA (1.0 mL) and the reaction mixture was stirred at 50° C. for 2 h. The resulting mixture was concentrated under reduced pressure and basified to pH=10 with NH₃.H₂O. The crude residue was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 23% B to 54% B in 7 min, 54% B; Wave Length: 220 nm]: to afford (1R,2S,3R,5R)-3-{4-amino-5-methylpyrrolo[2,3-d]pyrimidin-7-yl}-5-[({3-[(2-phenylethyl) amino]propyl}amino) methyl]cyclopentane-1,2-diol 467 (6.8 mg, 39%) as an off-white solid. ¹H NMR (400 MHZ, Chloroform-d) 88.22 (s, 1H), 7.36-7.30 (m, 2H), 7.27-7.20 (m, 3H), 6.73 (s, 1H), 5.16 (br s, 2H), 4.72-4.63 (m, 1H), 4.24-4.11 (m, 1H), 4.04-3.90 (m, 1H), 3.00-2.92 (m, 2H), 2.91-2.84 (m, 2H), 2.83-2.69 (m, 6H), 2.49-2.36 (m, 4H), 2.32-2.23 (m, 1H), 1.85-1.65 (m, 3H). LCMS [conditions ZORBAX SB-Aq, 50*4.6 mm, 1.8 μm, Mobile Phase A: Water/0.05% TFA, Mobile Phase B: Acetonitrile, 5%-95% B-3 min, 1.50 mL/min, ES, m/z]: T_{R}=0.95 min; [M+H]⁺: 439. Chiral-SFC [Column: Lux-4 100×4.6 mm 3.0 μm, Solvent B: MeOH (20 mM NH₃), Oven Temperature: 35° C., Total Flow: 3.00 mL/min]: T_{R}=1.48 min; single peak.

### Synthesis of (1R,2S,3R,5R)-3-(4-amino-5-cyclopropyl-7H-pyrrolo[2,3-d]pyrimidin-7-yl)-5-(((3-(phenethylamino)propyl) amino) methyl) cyclopentane-1,2-diol (468)

### tert-Butyl-(3-((((3αR,4R,6R,6αS)-6-(4-amino-5-cyclopropyl-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(phenethyl)carbamate (123a)

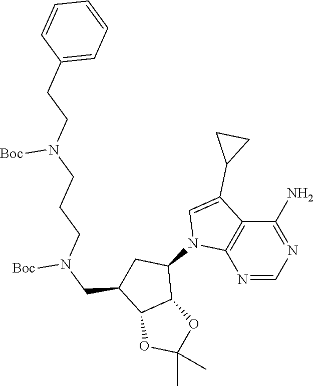 [SMILES: CC(C)(C)OC(=O)N(CCCN(C[C@H]1C[C@@H](n2cc(C3CC3)c3c(N)ncnc32)[C@@H]2OC(C)(C)O[C@H]12)C(=O)OC(C)(C)C)CCc1ccccc1]

To a solution of tert-butyl-NV-[3-({[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 108a (70 mg, 0.10 mmol, 1.0 eq) and cyclopropylboronic acid (16 mg, 0.20 mmol, 2 eq) in toluene (2.0 mL) and H₂O (0.2 mL) was added K₃PO₄ (60 mg, 0.30 mmol, 3.0 eq), Pd (AcO) 2 (4.0 mg, 0.02 mmol, 0.2 eq) and PCy₃ (10 mg, 0.04 mmol, 0.4 eq) and the reaction mixture was stirred at 100° C., for 2 h, under nitrogen. The resulting mixture was concentrated under reduced pressure and the crude residue purified by silica gel column chromatography (90:10 DCM: MeOH) to afford tert-butyl-N-[3-({[(3αR,4R,6R,6αS)-6-{4-amino-5-cyclopropylpyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 123a (60 mg, 90%) as a yellow solid. LCMS [XBridge C18, 50*3.0 mm, 3.5 μm; Mobile Phase A: Water/5 mM NH₄HCO₃, Mobile Phase B: MeOH, 10%-95% B-2 min (+), 1.00 mL/min, ES, m/z]: T_{R}=1.62 min; [M+H]⁺: 705.

### (1R,2S,3R,5R)-3-(4-Amino-5-cyclopropyl-7H-pyrrolo[2,3-d]pyrimidin-7-yl)-5-(((3-(phenethylamino)propyl) amino) methyl) cyclopentane-1,2-diol (468)

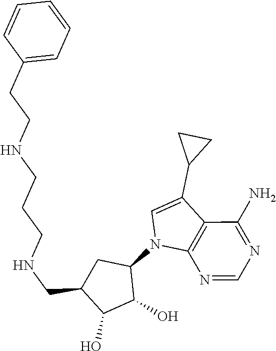 [SMILES: Nc1ncnc2c1c(C1CC1)cn2[C@@H]1C[C@H](CNCCCNCCc2ccccc2)[C@@H](O)[C@H]1O]

To tert-butyl-N-[3-({[(3αR,4R,6R,6αS)-6-{4-amino-5-cyclopropylpyrrolo[2,3-d]pyrimidin-7-yl}-2,2-dimethyl-tetrahydro-3αH-cyclopenta[d][1,3]-dioxol-4-yl]methyl}(tert-butoxycarbonyl) amino)propyl]-N-(2-phenylethyl)carbamate 123a (60 mg, 0.10 mmol, 1.0 eq) was added TFA (2.0 mL) at room temperature and the reaction mixture was stirred at 50° C. for 1 h. The resulting mixture was concentrated under reduced pressure and basified to pH=10 with NH₃.H₂O. The crude product was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 25% B to 55% B in 7 min, 55% B; Wave Length: 220 nm]: to afford (1R,2S,3R,5R)-3-{4-amino-5-cyclopropylpyrrolo[2,3-d]pyrimidin-7-yl}-5-[({3-[(2-phenylethyl) amino]propyl}amino) methyl]cyclopentane-1,2-diol 468 (14.9 mg, 38%) as an off-white solid. ¹H NMR (400 MHZ, DMSO-d₆) δ 8.00 (s, 1H), 7.33-7.12 (m, 5H), 6.96 (s, 1H), 6.51 (s, 2H), 4.90-4.68 (m, 1H), 4.24-4.04 (m, 1H), 3.78-3.69 (m, 1H), 2.87-2.49 (m, 10H), 2.19-1.94 (m, 3H), 1.62-1.53 (m, 2H), 1.49-1.33 (m, 1H), 0.94-0.78 (m, 2H), 0.64-0.49 (m, 2H). LCMS [conditions

Cortecs C_{18+, 50*3.0} mm, 2.7 μm, Mobile Phase A: Water/0.02% TFA, Mobile Phase B: Acetonitrile, 5%-95% B-3 min, 1.50 mL/min, ES, m/z]: T_{R}=0.99 min; [M+H]⁺: 465. Chiral-SFC [Column: Amylose C Neo 100×4.6 mm 3.0 μm, Solvent B: MeOH (20 mM NH₃), Oven Temperature: 35° C., Total Flow: 3.00 mL/min. Gradient 30% solvent B for 4 mins]: T_{R}=1.30 min (major peak), T_{R}=1.77 min (minor peak), dr=97.5:2.5.

### Synthesis of (1R,2S,3R,5R)-3-(4-amino-5-(sec-butyl)-7H-pyrrolo[2,3-d]pyrimidin-7-yl)-5-(((3-(phenethylamino)propyl) amino) methyl) cyclopentane-1,2-diol (469)

### tert-Butyl-(3-((((3αR,4R,6R,6αS)-6-(4-amino-5-((E)-but-2-en-2-yl)-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(phenethyl)carbamate (124a)

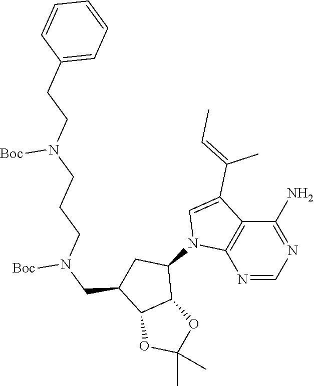 [SMILES: C/C=C(\C)c1cn([C@@H]2C[C@H](CN(CCCN(CCc3ccccc3)C(=O)OC(C)(C)C)C(=O)OC(C)(C)C)[C@H]3OC(C)(C)O[C@H]32)c2ncnc(N)c12]

To a solution of tert-butyl-N-[3-({[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 108a (90 mg, 0.12 mmol, 1.0 eq) and potassium (2-Z)-but-2-en-2-yltrifluoroboranuide (39 mg, 0.24 mmol, 2.0 eq) in toluene (2.5 mL) and H₂O (0.3 mL) were added K₃PO₄ (77 mg, 0.36 mmol, 3.0 eq), Pd (OAc) 2 (5 mg, 0.02 mmol, 0.2 eq) and PCy₃ (13 mg, 0.04 mmol, 0.4 eq) and the reaction mixture was stiired at 100° C. for 2 h, under nitrogen. The resulting mixture was concentrated under reduced pressure and the crude residue was purified by silica gel column chromatography (90:10 DCM: MeOH) to afford tert-butyl-N-[3-({[(3αR,4R,6R,6αS)-6-{4-amino-5-[(2E)-but-2-en-2-yl]pyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 124a (40 mg, 46%) as a yellow solid. LCMS [Xbridge Shield RP18, 50*3.0 mm, 3.5 μm; Mobile Phase A: Water/0.05% Ammonia, Mobile Phase B: Acetonitrile, 5%-100% B-2 min (+), 1.50 mL/min, ES, m/z]: T_{R}=1.46 min; [M+H]⁺: 719.

### tert-Butyl(3-((((3αR,4R,6R,6αS)-6-(4-amino-5-(sec-butyl)-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(phenethyl)carbamate (125a)

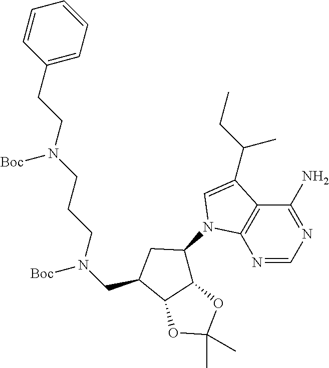 [SMILES: CCC(C)c1cn([C@@H]2C[C@H](CN(CCCN(CCc3ccccc3)C(=O)OC(C)(C)C)C(=O)OC(C)(C)C)[C@H]3OC(C)(C)O[C@H]32)c2ncnc(N)c12]

To a solution of tert-butyl-N-[3-({[(3αR,4R,6R,6αS)-6-{4-amino-5-[(2-E)-but-2-en-2-yl]pyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 124a (40 mg, 0.05 mmol, 1.0 eq) in 3.0 mL MeOH was added Pd/C(10%, 2 mg) and the reaction mixture was hydrogenated at room temperature for 1 h, under a hydrogen balloon. The resultant mixture was filtered through a Celite pad and concentrated under reduced pressure to afford tert-butyl-N-[3-({[(3αR,4R,6R,6αS)-6-[4-amino-5-(sec-butyl) pyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate 125a (30 mg, 75%) as a yellow solid. LCMS [XBridge C18, 50*3.0 mm, 3.5 μm; Mobile Phase A: Water/5 mM NH₄HCO₃, Mobile Phase B: MeOH, 10%-95% B-3 min (+), 1.50 mL/min, ES, m/z]: T_{R}=2.49 min; [M+H]⁺: 721.

### (1R,2S,3R,5R)-3-(4-Amino-5-(sec-butyl)-7H-pyrrolo[2,3-d]pyrimidin-7-yl)-5-(((3-(phenethylamino)propyl) amino) methyl) cyclopentane-1,2-diol (469)

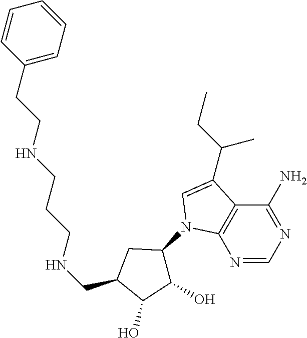 [SMILES: CCC(C)c1cn([C@@H]2C[C@H](CNCCCNCCc3ccccc3)[C@@H](O)[C@H]2O)c2ncnc(N)c12]

To tert-butyl-N-[3-({[(3αR,4R,6R,6αS)-6-[4-amino-5-(sec-butyl) pyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate 125a (30 mg, 0.04 mmol, 1.0 eq) was added TFA (2.0 mL), at room temperature and the reaction mixture was stirred at 50° C. for 1 h. The resulting mixture was concentrated under reduced pressure and basified to pH=10 with NH₃.H₂O. The crude product was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 22% B to 53% B in 7 min, 53% B; Wave Length: 220 nm]: to afford (1R,2S,3R,5R)-3-[4-amino-5-(sec-butyl) pyrrolo[2,3-d]pyrimidin-7-yl]-5-[({3-[(2-phenylethyl) amino]propyl}amino) methyl]cyclopentane-1,2-diol 469 (9.9 mg, 50%) as an off-white solid. ¹H NMR (400 MHZ, DMSO-d₆) § 8.00 (s, 1H), 7.33-7.23 (m, 2H), 7.21-7.13 (m, 3H), 7.01 (s, 1H), 6.36 (s, 2H), 4.88-4.78 (m, 1H), 4.71 (s, 1H), 4.18-4.10 (m, 1H), 3.81-3.71 (m, 1H), 3.15-3.06 (m, 1H), 2.82-2.65 (m, 6H), 2.63-2.54 (m, 4H), 2.20-2.09 (m, 1H), 2.07-1.94 (m, 1H), 1.79-1.67 (m, 1H), 1.58-1.40 (m, 4H), 1.25-1.16 (m, 3H), 0.94-0.82 (m, 3H). LCMS [conditions ZORBAX SB-Aq, 50*4.6 mm, 1.8 μm, Mobile Phase A: Water/0.02% TFA, Mobile Phase B: Acetonitrile, 5%-95% B-3 min, 1.50 mL/min, ES, m/z]: T_{R}=1.07 min; [M+H]⁺: 481. Chiral-SFC [Column: Lux-4 100×4.6 mm 3.0 μm, Solvent B: MeOH (20 mM NH₃), Oven Temperature: 35° C., Total Flow: 3.00 mL/min]: T_{R}=1.82 min, single peak.

### Synthesis of (1R,2S,3R,5R)-3-(4-amino-5-(pyridin-4-yl)-7H-pyrrolo[2,3-d]pyrimidin-7-yl)-5-(((3-(phenethylamino)propyl) amino) methyl) cyclopentane-1,2-diol (470)

### tert-Butyl-(3-((((3αR,4R,6R,6αS)-6-(4-amino-5-(pyridin-4-yl)-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(phenethyl)carbamate (126a)

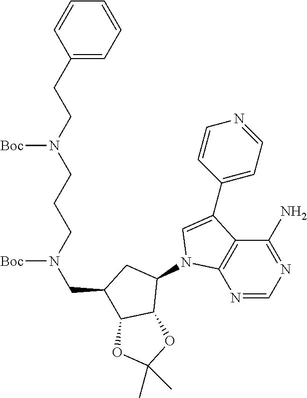 [SMILES: CC(C)(C)OC(=O)N(CCCN(C[C@H]1C[C@@H](n2cc(-c3ccncc3)c3c(N)ncnc32)[C@@H]2OC(C)(C)O[C@H]12)C(=O)OC(C)(C)C)CCc1ccccc1]

To a solution of tert-butyl-N-[3-({[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 108a (70 mg, 0.10 mmol, 1.0 eq) and pyridin-4-ylboronic acid (17 mg, 0.15 mmol, 1.5 eq) in dioxane (2.0 mL) and H₂O (0.2 mL) were added K₃PO₄ (60 mg, 0.30 mmol, 3.0 eq) and Pd (dtbpf) Cl₂ (12 mg, 0.02 mmol, 0.2 eq) and the reaction mixture was stirred at 80° C. for 1 h, under nitrogen. The resulting mixture was concentrated under reduced pressure and the crude residue was purified by silica gel column chromatography (90:10 DCM: MeOH) to afford tert-butyl-N-[3-({[(3αR,4R,6R,6αS)-6-[4-amino-5-(pyridin-4-yl) pyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate 126a (55 mg, 79%) as a yellow solid. LCMS [XBridge C18, 50*3.0 mm, 3.5 μm; Mobile Phase A: Water/5 mM NH₄HCO₃, Mobile Phase B: MeOH, 10%-95% B-3 min (+), 1.50 mL/min, ES, m/z]: T_{R}=2.41 min; [M+H]⁺: 742.

### (1R,2S,3R,5R)-3-(4-Amino-5-(pyridin-4-yl)-7H-pyrrolo[2,3-d]pyrimidin-7-yl)-5-(((3-(phenethylamino)propyl) amino) methyl) cyclopentane-1,2-diol (470)

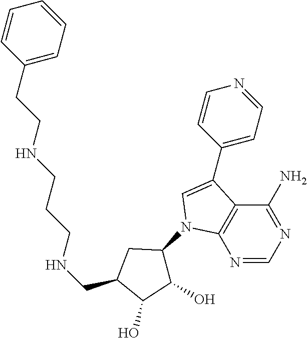 [SMILES: Nc1ncnc2c1c(-c1ccncc1)cn2[C@@H]1C[C@H](CNCCCNCCc2ccccc2)[C@@H](O)[C@H]1O]

To tert-butyl-N-[3-({[(3αR,4R,6R,6αS)-6-[4-amino-5-(pyridin-4-yl) pyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate 126a (55 mg, 0.08 mmol, 1.0 eq) was added TFA (2.0 mL) at room temperature and the reaction mixture was stirred at 50° C. for 1 h. The resulting mixture was concentrated under reduced pressure and basified to pH=10 with NH₃.H₂O. The crude residue was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 ml/min; Gradient: 28% B to 59% B in 7 min, 59% B; Wave Length: 220 nm]: to afford (1R,2S,3R,5R)-3-[4-amino-5-(pyridin-4-yl) pyrrolo[2,3-d]pyrimidin-7-yl]-5-[{{3-[(2-phenylethyl) amino]propyl}amino) methyl]cyclopentane-1,2-diol 470 (13.7 mg, 37%) as an off-white solid. ¹H NMR (400 MHZ, DMSO-d₆) δ 8.66-8.48 (m, 2H), 8.17 (s, 1H), 7.75 (s, 1H), 7.51-7.43 (m, 2H), 7.29-7.22 (m, 2H), 7.21-7.13 (m, 3H), 6.29 (s, 2H), 5.11-4.88 (m, 1H), 4.38-4.17 (m, 1H), 3.97-3.72 (m, 1H), 2.86-2.53 (m, 10H), 2.31-2.17 (m, 1H), 2.15-1.99 (m, 1H), 1.69-1.44 (m, 3H). LCMS [conditions ZORBAX SB-Aq, 50*4.6 mm, 1.8 μm, Mobile Phase A: Water/0.05% TFA, Mobile Phase B: Acetonitrile, 5%-95% B-3 min, 1.50 mL/min, ES, m/z]: T_{R}=0.90 min; [M+H]⁺: 502. Chiral-SFC [Column: SB 100×4.6 mm 3.0 μm, Solvent B: MeOH (20 mM NH₃), Oven Temperature: 35° C., Total Flow: 3.00 mL/min]: T_{R}=2.16 min, single peak.

### Synthesis of (1R,2S,3R,5R)-3-(4-amino-5-(pyrimidin-4-yl)-7H-pyrrolo[2,3-d]pyrimidin-7-yl)-5-(((3-(phenethylamino)propyl) amino) methyl) cyclopentane-1,2-diol (471)

### tert-Butyl-(3-((((3αR,4R,6R,6αS)-6-(4-amino-5-(pyrimidin-4-yl)-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(phenethyl)carbamate (127a)

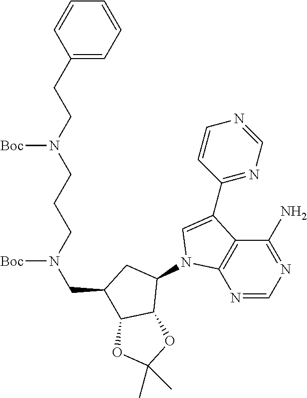 [SMILES: CC(C)(C)OC(=O)N(CCCN(C[C@H]1C[C@@H](n2cc(-c3ccncn3)c3c(N)ncnc32)[C@@H]2OC(C)(C)O[C@H]12)C(=O)OC(C)(C)C)CCc1ccccc1]

To tert-butyl-N-[3-({[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 108a (140 mg, 0.20 mmol, 1.0 eq), in dioxane (2.0 mL) at room temperature were added 4-(tributylstannyl) pyrimidine (45 mg, 0.60 mmol, 3.0 eq), Pd (PPh₃) 4 (11 mg, 0.009 mmol, 0.05 eq), LiCl (21 mg, 0.50 mmol, 2.5 eq) and Cul (2.0 mg, 0.009 mmol, 0.05 eq) and the reaction mixture was stirred at 120° C. for 12 h, under N₂. The resulting mixture was concentrated under vacuum and the crude residue was dissolved in AcOEt, filtered and the filter cake washed with AcOEt (3×10 mL). The combined washings were concentrated under reduced pressure and the crude residue was purified by Prep-TLC(80:20 AcOEt: petroleum ether) to afford tert-butyl-N-[3-({[(3αR,4R,6R,6αS)-6-[4-amino-5-(pyrimidin-4-yl) pyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate 127a (67 mg, 48%) as a white solid. LCMS [conditions: L-column3 ODS, 50*3.0 mm, 3.0 μm, Mobile Phase A: Water (0.05% ammonia water), Mobile Phase B: Acetonitrile, 5%-95% B-3 min (+).Icm, 1.50 mL/min]: T_{R}=2.22 min; [M+H]⁺: 743.

### (1R,2S,3R,5R)-3-(4-Amino-5-(pyrimidin-4-yl)-7H-pyrrolo[2,3-d]pyrimidin-7-yl)-5-(((3-(phenethylamino)propyl) amino) methyl) cyclopentane-1,2-diol (471)

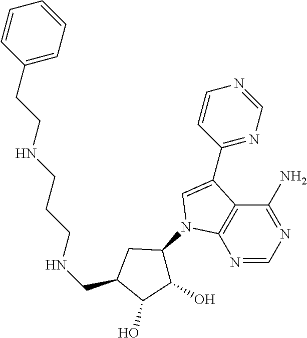 [SMILES: Nc1ncnc2c1c(-c1ccncn1)cn2[C@@H]1C[C@H](CNCCCNCCc2ccccc2)[C@@H](O)[C@H]1O]

To tert-butyl-N-[3-({[(3αR,4R,6R,6αS)-6-[4-amino-5-(pyrimidin-4-yl) pyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate 127a (67 mg, 0.09 mmol, 1.0 eq) was added TFA (2.0 mL) and the reaction mixture was stirred at 50° C. for 1 h. The resulting mixture was concentrated under reduced pressure and was basified to pH >8 with NH₃.H₂O. The crude residue was dissolved in DMF and purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 ml/min; Gradient: 20% B to 55% B in 7 min, 55% B; Wave Length: 220 nm]: to afford (1R,2S,3R,5R)-3-[4-amino-5-(pyrimidin-4-yl) pyrrolo[2,3-d]pyrimidin-7-yl]-5-[{3-[(2-phenylethyl) amino]propyl}amino) methyl]cyclopentane-1,2-diol 471 (23.2 mg, 51%) as a white solid. ¹H NMR (400 MHZ, DMSO-d₆) δ9.09 (d, J=1.3 Hz, 1H), 8.67 (d, J=5.7 Hz, 1H), 8.54 (s, 1H), 8.10 (s, 1H), 8.07-8.00 (m, 1H), 7.29-7.22 (m, 2H), 7.21-7.11 (m, 3H), 5.02-4.90 (m, 1H), 4.33-4.25 (m, 1H), 3.84-3.77 (m, 1H), 2.72-2.63 (m, 5H), 2.62-2.54 (m, 5H), 2.29-2.18 (m, 1H), 2.09-2.04 (m, 1H), 1.60-1.47 (m, 3H). LCMS [conditions: ZORBAX SB-Aq, 50*4.6 mm, 1.8 μm, Mobile Phase A: Water/0.05% TFA, Mobile Phase B: Acetonitrile, 5%-95% B-3 min, 1.50 mL/min]: T_{R}=0.10 min; [M+H]⁺: 503. Chiral HPLC [CHIRALPAK IJ-3, 100*4.6 mm, 3 μm, Mobile Phase A: Water/0.05% TFA, Mobile Phase B: CAN, Total Flow: 1.00 mL/min]: T_{R}=2.55 min, single peak.

### Synthesis of (1R,2S,3R,5R)-3-(4-amino-5-(1H-pyrazol-5-yl)-7H-pyrrolo[2,3-d]pyrimidin-7-yl)-5-(((3-(phenethylamino)propyl) amino) methyl) cyclopentane-1,2-diol (472)

### tert-Butyl-(3-((((3αR,4R,6R,6αS)-6-(4-amino-5-(1-(tetrahydro-2H-pyran-2-yl)-1H-pyrazol-5-yl)-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(phenethyl)carbamate (128a)

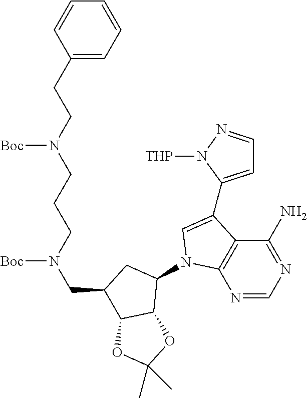

To a stirred mixture of tert-butyl-N-[3-({[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate (60 mg, 0.08 mmol, 1.0 eq) and 1-(oxan-2-yl)-5-(4,4,5,5-tetramethyl-1,3,2-dioxaborolan-2-yl) pyrazole 108a (34 mg, 0.12 mmol, 1.5 eq) in 1,4-dioxane (1.0 mL) and H₂O (0.1 mL) were added Pd (dtbpf) Cl₂ (2.6 mg, 0.004 mmol, 0.05 eq) and K₃PO₄ (52 mg, 0.24 mmol, 3.0 eq) and the reaction mixture was stirred at 80° C. for 1 h, under N₂. The resulting mixture was concentrated under reduced pressure and extracted with AcOEt (3×20 mL). The combined organic layers were washed with brine, dried over anhydrous Na₂SO₄, filtered and concentrated under reduced pressure. The crude residue was purified by silica gel column chromatography (50:50 tetrahydrofuran: petroleum ether) to afford tert-butyl-N-[3-({[(3αR,4R,6R,6αS)-6-{4-amino-5-[2-(oxan-2-yl) pyrazol-3-yl]pyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 128a (58 mg, 88%) as a light yellow solid. LCMS [conditions: XBridge C18, 50*3.0 mm, 3.5 μm, Mobile Phase A: Water/0.05% Ammonia, Mobile Phase B: Acetonitrile, 5%-100% B-2 min (+).lcm, 1.50 mL/min]: T_{R}=1.32 min; [M+H]⁺: 815.

### (1R,2S,3R,5R)-3-(4-Amino-5-(1H-pyrazol-5-yl)-7H-pyrrolo[2,3-d]pyrimidin-7-yl)-5-(((3-(phenethylamino)propyl) amino) methyl) cyclopentane-1,2-diol (472)

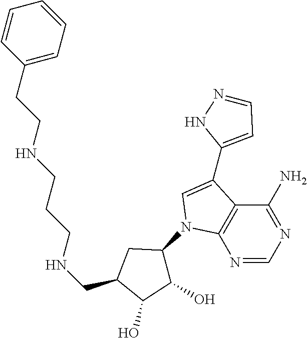 [SMILES: Nc1ncnc2c1c(-c1ccn[nH]1)cn2[C@@H]1C[C@H](CNCCCNCCc2ccccc2)[C@@H](O)[C@H]1O]

To tert-butyl-N-[3-({[(3αR,4R,6R,6αS)-6-{4-amino-5-[2-(oxan-2-yl) pyrazol-3-yl]pyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 128a (58 mg, 0.07 mmol, 1.0 eq) was added TFA (1.0 mL) and the reaction mixture was stirred at 50° C. for 1 h. The resulting mixture was concentrated under reduced pressure and was basified to pH >8 with NH₃.H₂O. The crude residue was dissolved in DMF and purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 25% B to 65% B in 7 min, 65% B; Wave Length: 220 nm]: to afford (1R,2S,3R,5R)-3-[4-amino-5-(2H-pyrazol-3-yl) pyrrolo[2,3-d]pyrimidin-7-yl]-5-[({3-[(2-phenylethyl) amino]propyl}amino) methyl]cyclopentane-1,2-diol 472 (15.3 mg, 44%) as an off-white solid. ¹H NMR (400 MHZ, DMSO-d₆) δ 12.81 (br s, 1H), 9.17 (br s, 1H), 8.03 (s, 1H), 7.84-7.71 (m, 2H), 7.36-7.02 (m, 6H), 6.73-6.61 (m, 1H), 4.99-4.72 (m, 2H), 4.27-4.17 (m, 1H), 3.86-3.77 (m, 1H), 3.01-2.57 (m, 10H), 2.26-2.15 (m, 1H), 2.12-2.02 (m, 1H), 1.64-1.46 (m, 3H). LCMS [conditions: ZORBAX SB-Aq, 50*4.6 mm, 1.8 μm, Mobile Phase A: Water/0.05% TFA, Mobile Phase B: Acetonitrile, 5%-95% B-3 min, 1.50 mL/min]: T_{R}=1.01 min; [M+H]⁺: 491. Chiral-SFC [Column: Lux-4 100×4.6 mm 3.0 μm, Solvent B: MeOH (20 mM NH₃), Oven Temperature: 35° C., Total Flow: 3.00 mL/min. Gradient 50% solvent B for 4 mins]: T_{R}=1.46 min, single peak.

### Synthesis of (1R,2S,3R,5R)-3-(4-amino-5-(1H-imidazol-2-yl)-7H-pyrrolo[2,3-d]pyrimidin-7-yl)-5-(((3-(phenethylamino)propyl) amino) methyl) cyclopentane-1,2-diol (473)

### tert-Butyl-(3-((tert-butoxycarbonyl)(phenethyl) amino)propyl)(((3αR,4R,6R,6αS)-6-(4-chloro-5-iodo-7H-pyrrolo[2,3-d]pyrimidin-7-yl)-2,2-dimethyltetrahydro-4H-cyclopenta[d][1,3]dioxol-4-yl)methyl)carbamate (129a)

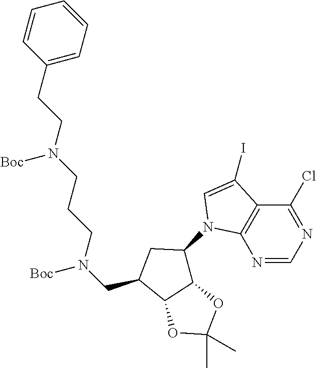 [SMILES: CC(C)(C)OC(=O)N(CCCN(C[C@H]1C[C@@H](n2cc(I)c3c(Cl)ncnc32)[C@@H]2OC(C)(C)O[C@H]12)C(=O)OC(C)(C)C)CCc1ccccc1]

To a stirred solution of tert-butyl-N-[3-({[(3αR,4R,6R,6a)-6-{4-chloropyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 106a (0.50 g, 0.70 mmol, 1.0 eq) and Na₂CO₃ (0.19 g, 1.80 mmol, 2.5 eq) in DCM (5.0 mL) was added, dropwise, ICl (1.8 mL, 1.80 mmol, 2.5 eq), at room temperature and the reaction mixture was stirred at room temperature for 4 h. The reaction was quenched with saturated sodium thiosulfate solution and was extracted with AcOEt (3×20 mL). The combined organic layers were washed with saturated sodium thiosulfate solution, saturated sodium bicarbonate solution and brine, then dried over anhydrous Na₂SO₄. After filtration, the filtrate was concentrated under reduced pressure and the crude residue was purified by silica gel column chromatography (70:30 tetrahydrofuran: petroleum ether) to afford tert-butyl-N-[3-({[(3αR,4R,6R,6αS)-6-{4-chloro-5-iodopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 129a (0.42 g, 71%) as a brown yellow solid. LCMS [conditions: XBridge C18, 50*3.0 mm, 3.5 μm, Mobile Phase A: Water/5 mM NH₄HCO₃, Mobile Phase B: Methanol, 10%-95% B-3 min (+).Icm, 1.50 mL/min]: T_{R}=2.62 min; [M+H]⁺: 810.

### tert-Butyl(((3αR,4R,6R,6αS)-6-(4-amino-5-iodo-7H-pyrrolo[2,3-d]pyrimidin-7-yl)-2,2-dimethyltetrahydro-4H-cyclopenta[d][1,3]dioxol-4-yl)methyl)(3-((tert-butoxycarbonyl)(phenethyl) amino)propyl)carbamate (130a)

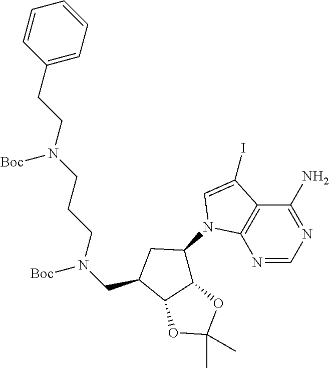 [SMILES: CC(C)(C)OC(=O)N(CCCN(C[C@H]1C[C@@H](n2cc(I)c3c(N)ncnc32)[C@@H]2OC(C)(C)O[C@H]12)C(=O)OC(C)(C)C)CCc1ccccc1]

To tert-butyl-N-[3-({[(3αR,4R,6R,6αS)-6-{4-chloro-5-iodopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 129a (0.42 g, 0.50 mmol, 1.0 eq) was added and NH₃ (7M in MeOH, 5.0 mL) at room temperature and the reaction mixture was stirred at 70° C. for 12 h, under N₂. The resulting mixture was concentrated under reduced pressure to afford crude tert-butyl-N-[3-({[(3αR,4R,6R,6αS)-6-{4-amino-5-iodopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 130a (0.33 g, 81%) which was used in the next step without further purification. TH NMR (400 MHZ, Chloroform-d) 8 8.24 (s, 1H), 7.36-7.29 (m, 2H), 7.26-7.13 (m, 4H), 4.62-4.45 (m, 1H), 3.45-3.10 (m, 8H), 2.97-2.74 (m, 3H), 2.56-2.32 (m, 3H), 1.95-1.66 (m, 3H), 1.55 (s, 3H), 1.46 (s, 18H), 1.28 (s, 3H).

### tert-Butyl-(((3αR,4R,6R,6αS)-6-(4-amino-5-(1-((2-(trimethylsilyl) ethoxy)methyl)-1H-imidazol-2-yl)-7H-pyrrolo[2,3-d]pyrimidin-7-yl)-2,2-dimethyltetrahydro-4H-cyclopenta[d][1,3]dioxol-4-yl)methyl)(3-((tert-butoxycarbonyl)(phenethyl) amino)propyl)carbamate (131a)

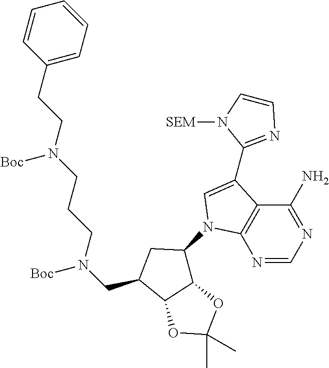 [SMILES: CC(C)(C)OC(=O)N(CCCN(C[C@H]1C[C@@H](n2cc(-c3nccn3COCC[Si](C)(C)C)c3c(N)ncnc32)[C@@H]2OC(C)(C)O[C@H]12)C(=O)OC(C)(C)C)CCc1ccccc1]

To a stirred solution of chloro (1-{[2-(trimethylsilyl) ethoxy]methyl}imidazol-2-yl) zinc 133a (2.0 mL, 0.18 mol/L, 1.8 eq) was added, dropwise, a solution of tert-butyl-N-[3-({[(3αR,4R,6R,6αS)-6-{4-amino-5-iodopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 130a (0.10 g, 0.10 mmol, 1.0 eq), Pd₂ (dba)₃.CHCl₃ (13 mg, 0.013 mmol, 0.1 eq), tris (furan-2-yl) phosphane (6.0 mg, 0.03 mmol, 0.2 eq) and K₃PO₄ (81 mg, 0.40 mmol, 3.0 eq) in THF (2.0 mL) at room temperature under N₂ and the reaction mixture was stirred at 60° C. for 12 h. The reaction was quenched with saturated ammonium chloride solution and extracted with AcOEt (3×20 mL). The combined organic layers were washed with brine, dried over anhydrous Na₂SO₄, filtered and concentrated under reduced pressure. The crude residue was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 30% B to 85% B in 7 min, 85% B; Wave Length: 220 nm]: to afford tert-butyl-N-[3-({[(3αR,4R,6R,6αS)-6-[4-amino-5-(1-{[2-(trimethylsilyl) ethoxy]methyl}imidazol-2-yl) pyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate 131a (15 mg, 14%) as a white solid. LCMS [conditions: Cortecs C_{18+, 50*3.0} mm, 2.7 μm, Mobile Phase A: Water/0.05% TFA, Mobile Phase B: Acetonitrile, 2%-80% B-5 min (+).Icm, 1.50 mL/min]: T_{R}=3.83 min; [M+H]⁺: 861.

### (1R,2S,3R,5R)-3-(4-Amino-5-(1H-imidazol-2-yl)-7H-pyrrolo[2,3-d]pyrimidin-7-yl)-5-(((3-(phenethylamino)propyl) amino) methyl) cyclopentane-1,2-diol (473)

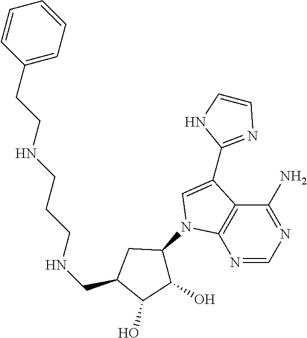 [SMILES: Nc1ncnc2c1c(-c1ncc[nH]1)cn2[C@@H]1C[C@H](CNCCCNCCc2ccccc2)[C@@H](O)[C@H]1O]

To tert-butyl-N-[3-({[(3αR,4R,6R,6αS)-6-[4-amino-5-(1-{[2-(trimethylsilyl) ethoxy]methyl}imidazol-2-yl) pyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate 131a (15 mg, 0.017 mmol, 1.0 eq) was added TFA (0.5 mL) and the reaction mixture was stirred at 50° C. for 0.5 h. The resulting mixture was concentrated under reduced pressure and basified to pH >8 with NH₃.H₂O and the crude residue was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 ml/min; Gradient: 24% B to 49% B in 7 min, 49% B; Wave Length: 220 nm]: to afford (1R,2S,3R,5R)-3-[4-amino-5-(1H-imidazol-2-yl) pyrrolo[2,3-d]pyrimidin-7-yl]-5-[({3-[(2-phenylethyl) amino]propyl}amino) methyl]cyclopentane-1,2-diol 473 (4.5 mg, 53%) as an off-white solid. ¹H NMR (300 MHZ, DMSO-d₆+D₂O) 88.03 (s, 1H), 7.94-7.78 (m, 1H), 7.36-7.07 (m, 6H), 7.05-6.89 (m, 1H), 5.04-4.75 (m, 1H), 4.24-4.02 (m, 1H), 3.91-3.75 (m, 1H), 3.40-2.57 (m, 10H), 2.35-2.19 (m, 1H), 2.17-1.97 (m, 1H), 1.73-1.33 (m, 3H). LCMS [conditions: ZORBAX SB-Aq, 50*4.6 mm, 1.8 μm, Mobile Phase A: Water/0.02% TFA, Mobile Phase B: MeOH, 5%-95% B-5 min, 1.50 mL/min]: T_{R}=1.86 min; [M+H]⁺: 491. Chiral SFC [CHIRALPACK IF-3 50*3.0 mm,3.0 μm, Solvent A: CO₂, Solvent B: MeOH (0.2% MSA), Total Flow: 1.00 mL/min. Gradient: 10% to 50% in 2.0 min, hold 1.0 min at 50%]: T_{R}=1.69 min; single peak.

### Synthesis of (1-((2-(trimethylsilyl) ethoxy)methyl)-1H-imidazol-2-yl) zinc (II) chloride (41a) 2-Bromo-1-((2-(trimethylsilyl) ethoxy)methyl)-1H-imidazole (132a)

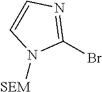 [SMILES: C[Si](C)(C)CCOCn1ccnc1Br]

To a stirred suspension of 2-bromo-1H-imidazole (0.50 g, 3.40 mmol, 1.0 eq) and K₂CO₃ (0.94 g, 6.80 mmol, 2.0 eq) in acetone (5.0 mL) was added SEMCl (0.68 g, 4.10 mmol, 1.2 eq) in acetone (3.0 mL) dropwise at room temperature and the reaction mixture was stirred at 28° C. for 12 h. The resulting mixture was concentrated under vacuum and the resultant residue was dissolved in AcOEt (60 mL) and then washed with brine, dried over anhydrous Na₂SO₄ and filtered. The filtrate was concentrated under reduced pressure and the crude residue was purified by silica gel column chromatography, (40:60 AcOEt: petroleum ether) to afford 2-bromo-1-{[2-(trimethylsilyl) ethoxy]methyl}imidazole 133a (0.50 g, 53%) as a colorless oil.

### (1-((2-(Trimethylsilyl) ethoxy)methyl)-1H-imidazol-2-yl) zinc (II) chloride (133a)

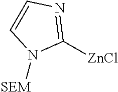

To a stirred solution of 2-bromo-1-{[2-(trimethylsilyl) ethoxy]methyl}imidazole 132a (0.30 g, 1.10 mmol, 1.0 eq) in THF (3.0 mL) was added n-BuLi (0.48 mL, 1.20 mmol, 1.1 eq), dropwise, at −78° C. under N₂ and the reaction mixture was stirred at −78° C. for 0.5 h (the reaction progress was monitored by deuterium methanol quenching an alequote of the reaction mixture). To the resulting mixture was added ZnCl2 (1.86 mL, 1.30 mmol, 1.2 eq) dropwise at −40° C. and the resulting mixture was stirred for additional 1 h at −20° C. (monitored by Deuterium methanol quenching) and the crude mixture of (1-((2-(trimethylsilyl) ethoxy)methyl)-1H-imidazol-2-yl) zinc (II) chloride 133a was used in the next step without further purification.

### Synthesis of (1R,2S,3R,5R)-3-(4-amino-5-(thiazol-2-yl)-7H-pyrrolo[2,3-d]pyrimidin-7-yl)-5-(((3-(phenethylamino)propyl) amino) methyl) cyclopentane-1,2-diol (474)

### tert-Butyl-(3-((((3αR,4R,6R,6αS)-6-(4-amino-5-(thiazol-2-yl)-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(phenethyl)carbamate (134a)

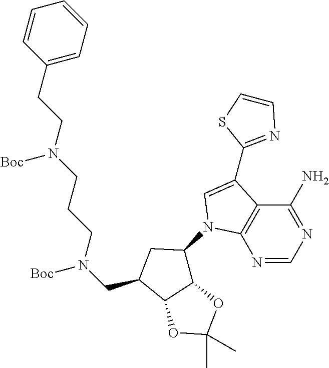 [SMILES: CC(C)(C)OC(=O)N(CCCN(C[C@H]1C[C@@H](n2cc(-c3nccs3)c3c(N)ncnc32)[C@@H]2OC(C)(C)O[C@H]12)C(=O)OC(C)(C)C)CCc1ccccc1]

To tert-butyl-N-[3-({[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 108a (80 mg, 0.11 mmol, 1.0 eq) in toluene (1.0 mL) at room temperature was added 2-(tributylstannyl)-1,3-thiazole (0.12 g, 0.33 mmol, 3.0 eq) and Pd (PPh₃) 2Cl₂ (4.0 mg, 0.005 mmol, 0.05 eq) and the reaction mixture was stirred at 120° C. for 12 h, under N₂. The resulting mixture was concentrated under vacuum and AcOEt (20 mL) was added. The resulting suspension was filtered through diatomite and the filter cake was washed with AcOEt (3×10 mL). The combined filtrate was concentrated under reduced pressure and the crude residue was purified by Prep-TLC(80:20 AcOEt: petroleum ether) to afford tert-butyl-N-[3-({[(3αR,4R,6R,6αS)-6-[4-amino-5-(1,3-thiazol-2-yl) pyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate 134a (63 mg, 78%) as a light yellow solid. LCMS [conditions: L-column3 ODS, 50*3.0 mm, 3.0 μm, Mobile Phase A: Water/0.05% ammonia water, Mobile Phase B: Acetonitrile, 5%-100% B-2 min (+).lcm, 1.50 mL/min]: T_{R}=1.57 min; [M+H]⁺: 748.

### (1R,2S,3R,5R)-3-(4-Amino-5-(thiazol-2-yl)-7H-pyrrolo[2,3-d]pyrimidin-7-yl)-5-(((3-(phenethylamino)propyl) amino) methyl) cyclopentane-1,2-diol (474)

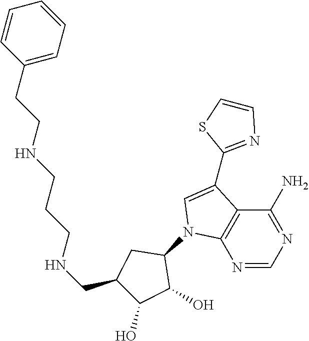 [SMILES: Nc1ncnc2c1c(-c1nccs1)cn2[C@@H]1C[C@H](CNCCCNCCc2ccccc2)[C@@H](O)[C@H]1O]

To tert-butyl-N-[3-({[(3αR,4R,6R,6αS)-6-[4-amino-5-(1,3-thiazol-2-yl) pyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate 134a (63 mg, 0.08 mmol, 1.0 eq) was added TFA (2.0 mL) and the reaction mixture was stirred at 50° C. for 1 h. The resulting mixture was concentrated under reduced pressure and the residue was basified to pH >8 with NH₃.H₂O and dissolved in DMF (3.0 mL). The crude product was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 ml/min; Gradient: 20% B to 55% B in 7 min, 55% B; Wave Length: 220 nm]: to afford (1R,2S,3R,5R)-3-[4-amino-5-(1,3-thiazol-2-yl) pyrrolo[2,3-d]pyrimidin-7-yl]-5-[{{3-[(2-phenylethyl) amino]propyl}amino) methyl]cyclopentane-1,2-diol 474 (25.5 mg, 60%) as an off-white solid. ¹H NMR (400 MHZ, DMSO-d₆+D₂O) 88.15 (s, 1H), 8.09 (s, 1H), 7.81 (d, J=3.4 Hz, 1H), 7.59 (d, J=3.4 Hz, 1H), 7.29-7.22 (m, 2H), 7.19-7.11 (m, 3H), 4.97-4.86 (m, 1H), 4.33-4.25 (m, 1H), 3.83-3.76 (m, 1H), 2.87-2.49 (m, 10H), 2.26-2.14 (m, 1H), 2.13-1.97 (m, 1H), 1.63-1.50 (m, 3H). LCMS [conditions: ZORBAX SB-Aq, 50*4.6 mm, 1.8 μm, Mobile Phase A: Water/0.05% TFA, Mobile Phase B: Acetonitrile, 5%-95% B-3 min, 1.50 mL/min]: T_{R}=1.04 min; [M+H]⁺: 508. Chiral HPLC [CHIRALPAK IC-3, 50*4.6 mm, 3 μm, Mobile Phase A: Water/0.05% TFA, Mobile Phase B: CAN, Total Flow: 1.00 mL/min]: T_{R}=1.17 min; single peak.

### Synthesis of (1R,2S,3R,5R)-3-(4-amino-5-(1H-1,2,4-triazol-5-yl)-7H-pyrrolo[2,3-d]pyrimidin-7-yl)-5-(((3-(phenethylamino)propyl) amino) methyl) cyclopentane-1,2-diol (475)

### tert-Butyl-(((3αR,4R,6R,6αS)-6-(4-amino-5-(1-(tetrahydro-2H-pyran-2-yl)-1H-1,2,4-triazol-5-yl)-7H-pyrrolo[2,3-d]pyrimidin-7-yl)-2,2-dimethyltetrahydro-4H-cyclopenta[d][1,3]dioxol-4-yl)methyl)(3-((tert-butoxycarbonyl)(phenethyl) amino)propyl)carbamate (135a)

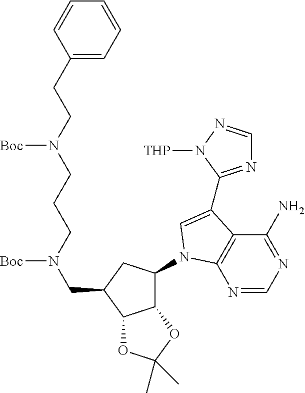

A crude sample of (1-(tetrahydro-2H-pyran-2-yl)-1H-1,2,4-triazol-5-yl) zinc (II) chloride 136a (1.5 mL, 0.35 mol/L in THF) was added dropwise, at room temperature under N₂, to a solution of tert-butyl-N-[3-({[(3αR,4R,6R,6αS)-6-{4-amino-5-iodopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 130a (50 mg, 0.06 mmol, 1.0 eq), Pd₂ (dba)₃·CHCl₃ (7.0 mg, 0.006 mmol, 0.1 eq), tris (furan-2-yl) phosphane (3 mg, 0.013 mmol, 0.2 eq) and K₃PO₄ (41 mg, 0.20 mmol, 3.0 eq) in THF (1.0 mL) and the reaction mixture was stirred at 60° C. for 12 h. The reaction was quenched with saturated ammonium chloride solution and the resulting mixture was extracted with AcOEt (3×20 mL). The combined organic layers were washed with brine, dried over anhydrous Na₂SO₄ and filtered. The filtrate was concentrated under reduced pressure and the crude residue was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 30% B to 80% B in 7 min, 80% B; Wave Length: 220 nm]: to afford tert-butyl-N-[3-({[(3αR,4R,6R,6αS)-6-{4-amino-5-[2-(oxan-2-yl)-1,2,4-triazol-3-yl]pyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 135a (25 mg, 49%) as a white solid. LCMS [conditions: Cortecs C_{18+, 50*3.0} mm, 2.7 μm, Mobile Phase A: Water/0.05% TFA, Mobile Phase B: Acetonitrile, 2%-95% B-3 min (+), 1.20 mL/min]: T_{R}=2.15 min; [M+H]⁺: 816.

### (1R,2S,3R,5R)-3-(4-Amino-5-(1H-imidazol-2-yl)-7H-pyrrolo[2,3-d]pyrimidin-7-yl)-5-(((3-(phenethylamino)propyl)amino)methyl)cyclopentane-1.7-diol (475)

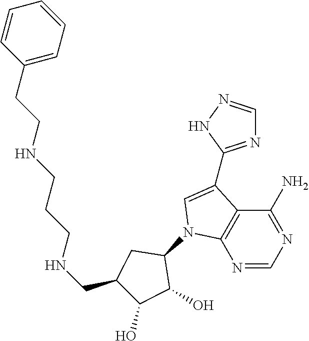 [SMILES: Nc1ncnc2c1c(-c1ncn[nH]1)cn2[C@@H]1C[C@H](CNCCCNCCc2ccccc2)[C@@H](O)[C@H]1O]

To tert-butyl-N-[3-({[(3αR,4R,6R,6αS)-6-{4-amino-5-[2-(oxan-2-yl)-1,2,4-triazol-3-yl]pyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 135a (25 mg, 0.025 mmol, 1.0 eq) was added TFA (0.5 mL) and the reaction mixture was stirred at 50° C. for 0.5 h. The resulting mixture was concentrated under reduced pressure, basified to pH >8 with NH₃.H₂O and the crude residue was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 20% B to 50% B in 7 min, 50% B; Wave Length: 220 nm]: to afford (1R,2S,3R,5R)-3-[4-amino-5-(2H-1,2,4-triazol-3-yl) pyrrolo[2,3-d]pyrimidin-7-yl]-5-[{{3-[(2-phenylethyl) amino]propyl}amino) methyl]cyclopentane-1,2-diol 475 (5.0 mg, 42%) as an off-white solid. ¹H NMR (300 MHZ, DMSO-d₆+D₂O) 88.39-8.24 (m, 1H), 8.08 (s, 1H), 8.05-7.89 (m, 1H), 7.33-7.09 (m, 5H), 4.94 (br s, 1H), 4.19 (br s, 1H), 3.80 (br s, 1H), 3.35-2.79 (m, 2H), 2.75-2.51 (m, 8H), 2.31-2.17 (m, 1H), 2.13-1.97 (m, 1H), 1.73-1.42 (m, 3H). LCMS [conditions: ZORBAX SB-Aq, 50*4.6 mm, 1.8 μm, Mobile Phase A: Water/0.05% ammonia water, Mobile Phase B: Acetonitrile, 5%-95% B-3 min, 1.50 mL/min]: T_{R}=1.03 min; [M+H]⁺: 492. Chiral HPLC [Cellulose SC, 100*4.6 mm, 3 μm, Mobile Phase A: Water/0.05% MSA, Mobile Phase B: CAN, Total Flow: 1.00 mL/min. gradient 10% B in 10 min]: T_{R}=1.75 min (major isomer), T_{R}=2.18 min (minor isomer); dr =98.7:1.3.

### Synthesis of (1-(tetrahydro-2H-pyran-2-yl)-1H-1,2,4-triazol-5-yl) zinc (II) chloride (136a) 1-(Tetrahydro-2H-pyran-2-yl)-1H-1,2,4-triazole (137a)

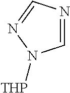

To a stirred mixture of 1,2,4-triazole (0.50 g, 7.20 mmol, 1.0 eq) and dihydropyran (0.91 g, 10.9 mmol, 1.5 eq) in THF (5.0 mL) was added imidazole (0.74 g, 10.9 mmol, 1.5 eq) at room temperature and the reaction mixture was stirred at 70° C. for 2 h. The crude mixture was purified by silica gel column chromatography (35:65 AcOEt: petroleum ether) to afford 1-(oxan-2-yl)-1,2,4-triazole 137a (0.60 g, 54%) as a colourless oil.

### (1-(Tetrahydro-2H-pyran-2-yl)-1H-1,2,4-triazol-5-yl) zinc (II) chloride (136a)

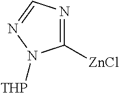

To a stirred solution of 1-(oxan-2-yl)-1,2,4-triazole 137a (0.20 g, 1.30 mmol, 1.0 eq) in THF (2.0 mL) was added n-BuLi (0.57 mL, 1.40 mmol, 1.1 eq) dropwise at −78° C. under N₂ and the resulting mixture was stirred at −78° C. for 0.5 h (the reaction progression was monitored by deuterium-methanol quenching). To the above mixture was added ZnCl₂ (2.24 mL, 1.60 mmol, 1.2 eq), dropwise, at −40° C. The reaction mixture was stirred for additional 1 h at −20° C. (reaction progress was monitored by deuterium-methanol quenching) and the crude sample of (1-(tetrahydro-2H-pyran-2-yl)-1H-1,2,4-triazol-5-yl) zinc (II) chloride 136a was used in the next step directly without isolation or purification.

### Synthesis of (1R,2S,3R,5R)-3-(4-amino-5-(1-methyl-1H-pyrazol-3-yl)-7H-pyrrolo[2,3-d]pyrimidin-7-yl)-5-(((3-(phenethylamino)propyl) amino) methyl) cyclopentane-1,2-diol (476)

### tert-Butyl-(((3αR,4R,6R,6αS)-6-(4-amino-5-(1-methyl-1H-pyrazol-3-yl)-7H-pyrrolo[2,3-d]pyrimidin-7-yl)-2,2-dimethyltetrahydro-4H-cyclopenta[d][1,3]dioxol-4-yl)methyl)(3-((tert-butoxycarbonyl)(phenethyl) amino)propyl)carbamate (170a)

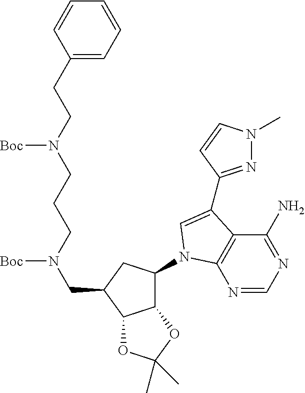 [SMILES: Cn1ccc(-c2cn([C@@H]3C[C@H](CN(CCCN(CCc4ccccc4)C(=O)OC(C)(C)C)C(=O)OC(C)(C)C)[C@H]4OC(C)(C)O[C@H]43)c3ncnc(N)c23)n1]

To a stirred mixture of tert-butyl-N-[3-({[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 108a (35 mg, 0.05 mmol, 1.0 eq) and 1-methyl-3-(4,4,5,5-tetramethyl-1,3,2-dioxaborolan-2-yl) pyrazole (20 mg, 0.10 mmol, 2.0 eq) in 1,4-dioxane (1.0 mL) and H₂O (0.1 mL) was added Pd (dtbpf) Cl₂ (1.5 mg, 0.002 mmol, 0.05 eq) and K₃PO₄ (30 mg, 0.10 mmol, 3.0 eq) and the reaction mixture was stirred at 90° C. for 0.5 h, under N₂. The resulting mixture was concentrated under reduced pressure and partitioned between AcOEt and brine. The organic layer was dried over anhydrous Na₂SO₄, filtered and the filtrate was concentrated under reduced pressure. The crude residue was purified by Prep-TLC (60:40 THF: petroleum ether) to afford tert-butyl-N-[3-({[(3αR,4R,6R,6αS)-6-[4-amino-5-(1-methylpyrazol-3-yl) pyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate 170a (30 mg, 86%) as a light yellow solid. LCMS [XBridge C18, 50*3.0 mm, 3.5 μm; Mobile Phase A: Water/5 mM NH₄HCO₃, Mobile Phase B: MeOH, 10%-95% B-3 min, 1.00 mL/min, ES, m/z]: T_{R}=2.44 min; [M+H]⁺: 745.

### (1R,2S,3R,5R)-3-(4-Amino-5-(1-methyl-1H-pyrazol-3-yl)-7H-pyrrolo[2,3-d]pyrimidin-7-yl)-5-(((3-(phenethylamino)propyl) amino) methyl) cyclopentane-1,2-diol (476)

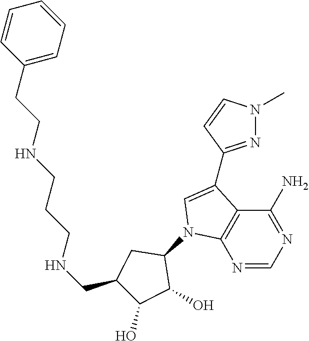 [SMILES: Cn1ccc(-c2cn([C@@H]3C[C@H](CNCCCNCCc4ccccc4)[C@@H](O)[C@H]3O)c3ncnc(N)c23)n1]

To tert-butyl-N-[3-({[(3αR,4R,6R,6αS)-6-[4-amino-5-(1-methylpyrazol-3-yl) pyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate (30 mg, 0.04 mmol, 1.0 eq) was added TFA (1.0 mL) and the reaction mixture was stirred at 50° C. for 1 h. The resulting mixture was concentrated under reduced pressure and was basified to pH >8 with NH₃.H₂O. The crude residue was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 20% B to 55% B in 9 min, 55% B; Wave Length: 220 nm]: to afford (1R,2S,3R,5R)-3-[4-amino-5-(1-methylpyrazol-3-yl) pyrrolo[2,3-d]pyrimidin-7-yl]-5-[({3-[(2-phenylethyl) amino]propyl}amino) methyl]cyclopentane-1,2-diol 476 (12.3 mg, 61%) as a white solid. ¹H NMR (400 MHZ, DMSO-d₆+D₂O) 88.02 (s, 1H), 7.77 (s, 1H), 7.70 (d, J=2.3 Hz, 1H), 7.30-7.21 (m, 2H), 7.20-7.11 (m, 3H), 6.63 (d, J=2.3 Hz, 1H), 4.94-4.82 (m, 1H), 4.26-4.16 (m, 1H), 3.87 (s, 3H), 3.81-3.75 (m, 1H), 2.82-2.51 (m, 10H), 2.25-2.14 (m, 1H), 2.10-2.00 (m, 1H), 1.63-1.41 (m, 3H). LCMS [conditions: ZORBAX SB-Aq, 50*4.6 mm, 1.8 μm, Mobile Phase A: Water/0.05% TFA, Mobile Phase B: Acetonitrile, 5%-95% B-3 min, 1.50 mL/min]: T_{R}=1.01 min; [M+H]⁺: 505. Chiral SFC [condition: Column Name: CHIRALPACK IF-3 50*3.0 mm,3.0 μm, Co-Solvent: MeOH: DCM=7:3 (20 mM NH₃), Gradient (B %): 10% to 50% in 2.0 min, hold 1.0 min at 50%, Flow (mL/min): 2.00 mL/min]: T_{R}=2.23 min, single peak.

### Synthesis of (1R,2S,3R,5R)-3-(4-amino-5-(1-benzyl-1H-pyrazol-3-yl)-7H-pyrrolo[2,3-d]pyrimidin-7-yl)-5-(((3-(phenethylamino)propyl) amino) methyl) cyclopentane-1,2-diol (477)

### tert-Butyl-(((3αR,4R,6R,6αS)-6-(4-amino-5-(1-benzyl-1H-pyrazol-3-yl)-7H-pyrrolo[2,3-d]pyrimidin-7-yl)-2,2-dimethyltetrahydro-4H-cyclopenta[d][1,3]dioxol-4-yl)methyl)(3-((tert-butoxycarbonyl)(phenethyl) amino)propyl)carbamate (171a)

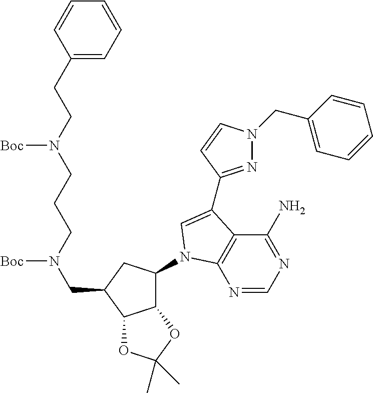 [SMILES: CC(C)(C)OC(=O)N(CCCN(C[C@H]1C[C@@H](n2cc(-c3ccn(Cc4ccccc4)n3)c3c(N)ncnc32)[C@@H]2OC(C)(C)O[C@H]12)C(=O)OC(C)(C)C)CCc1ccccc1]

To a stirred mixture of tert-butyl-N-[3-({[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 108a (40 mq, 0.05 mmol, 1.0 eq) and 1-benzyl-3-(4,4,5,5-tetramethyl-1,3,2-dioxaborolan-2-yl) pyrazole (31 mg, 0.10 mmol, 2.0 eq) in 1,4-dioxane (1.0 mL) and H₂O (0.1 mL) were added Pd (dtbpf) Cl₂ (2.0 mq, 0.003 mmol, 0.05 eq) and K₃PO₄ (34 mq, 0.20 mmol, 3.0 eq) and the reaction mixture was stirred at 90° C. for 0.5 h, under N₂. The resulting mixture was concentrated under reduced pressure and partitioned between AcOEt and brine. The organic layer was dried over anhydrous Na₂SO₄, filtered and the filtrate was concentrated under reduced pressure. The crude residue was purified by Prep-TLC (60:40 THF: petroleum ether) to afford tert-butyl-N-[3-({[(3αR,4R,6R,6αS)-6-[4-amino-5-(1-benzylpyrazol-3-yl) pyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate 171a (33 mg, 75%) as a light yellow solid. LCMS [L-column3 ODS, 50*3.0 mm, 3.0 μm; Mobile Phase A: Water/5 mM NH₄HCO₃+0.05% Ammonia, Mobile Phase B: Acetonitrile/5% water, 5%-100% B-2 min (+), 1.20 mL/min, ES, m/z]: T_{R}=1.71 min; [M+H]⁺: 821.

### (1R,2S,3R,5R)-3-(4-Amino-5-(1-benzyl-1H-pyrazol-3-yl)-7H-pyrrolo[2,3-d]pyrimidin-7-yl)-5-(((3-(phenethylamino)propyl) amino) methyl) cyclopentane-1,2-diol (477)

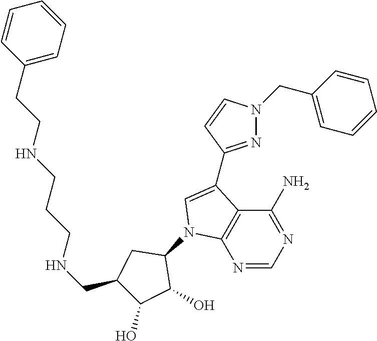 [SMILES: Nc1ncnc2c1c(-c1ccn(Cc3ccccc3)n1)cn2[C@@H]1C[C@H](CNCCCNCCc2ccccc2)[C@@H](O)[C@H]1O]

To tert-butyl-N-[3-({[(3αR,4R,6R,6αS)-6-[4-amino-5-(1-benzylpyrazol-3-yl) pyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate 171a (33 mg, 0.04 mmol, 1.0 eq) was added TFA (1.0 mL) and the reaction mixture was stirred at 50° C. for 1 h. The resulting mixture was concentrated under reduced pressure and was basified to pH >8 with NH₃.H₂O. The crude residue was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 25% B to 55% B in 7 min, 55% B; Wave Length: 220 nm]: to afford (1R,2S,3R,5R)-3-[4-amino-5-(1-benzylpyrazol-3-yl) pyrrolo[2,3-d]pyrimidin-7-yl]-5-[({3-[(2-phenylethyl) amino]propyl}amino) methyl]cyclopentane-1,2-diol 477 (16 mg, 69%) as a white solid. ¹H NMR (400 MHZ, DMSO-d₆) δ 8.09 (s, 1H), 7.97 (s, 1H), 7.60 (s, 1H), 7.38-7.34 (m, 3H), 7.32-7.28 (m, 3H), 7.27-7.22 (m, 2H), 7.19-7.12 (m, 3H), 5.37 (s, 2H), 4.94-4.84 (m, 1H), 4.21-4.13 (m, 1H), 3.79-3.72 (m, 1H), 2.72-2.62 (m, 5H), 2.57-2.49 (m, 5H), 2.22-2.14 (m, 1H), 2.09-1.98 (m, 1H), 1.60-1.43 (m, 3H). LCMS [conditions: ZORBAX SB-Aq, 50*4.6 mm, 1.8 μm, Mobile Phase A: Water/0.05% TFA, Mobile Phase B: Acetonitrile, 5%-95% B-3 min, 1.50 mL/min]: T_{R}=1.40 min; [M+H]⁺: 581. CHIRAL_HPLC [Column: CHIRALPAK ID-3, 50*4.6 mm, 3 μm, 25 dergree, Solvent B: MeOH (0.2% MSA), Solvent A: MTBE, keep Gradient 30% solvent B for 4 mins]: T_{R}=1.92 min, single peak.

### Synthesis of (1R,2S,3R,5R)-3-(4-amino-5-(1-methyl-1H-pyrazol-4-yl)-7H-pyrrolo[2,3-d]pyrimidin-7-yl)-5-(((3-(phenethylamino)propyl) amino) methyl) cyclopentane-1,2-diol (478)

### tert-Butyl-(3-((((3αR,4R,6R,6αS)-6-(4-amino-5-(1-methyl-1H-pyrazol-4-yl)-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(phenethyl)carbamate (172a)

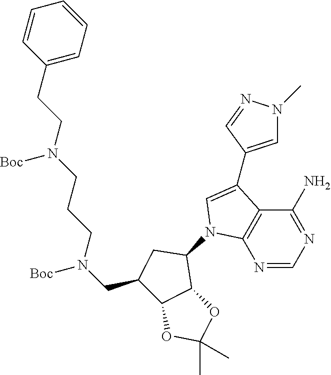 [SMILES: Cn1cc(-c2cn([C@@H]3C[C@H](CN(CCCN(CCc4ccccc4)C(=O)OC(C)(C)C)C(=O)OC(C)(C)C)[C@H]4OC(C)(C)O[C@H]43)c3ncnc(N)c23)cn1]

To a solution of tert-butyl-N-[3-({[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 108a (50 mg, 0.07 mmol, 1.0 eq) and 1-methyl-4-(4,4,5,5-tetramethyl-1,3,2-dioxaborolan-2-yl) pyrazole (21 mg, 0.10 mmol, 1.5 eq) in dioxane (1.0 mL) and H₂O (0.1 mL) were added K₃PO₄ (41 mg, 0.20 mmol, 3.0 eq) and Pd (dtbpf) Cl₂ (9.0 mg, 0.01 mmol, 0.2 eq) and the reaction mixture was stirred at 90° C. for 0.5 h, under nitrogen. The resulting mixture was concentrated under reduced pressure and the crude residue was purified by silica gel column chromatography (95:5 DCM: MeOH) to afford tert-butyl-N-[3-({[(3αR,4R,6R,6αS)-6-[4-amino-5-(1-methylpyrazol-4-yl) pyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate 172a (50 mg, 99%) as a yellow oil. LCMS [XBridge C18, 50*3.0 mm, 3.5 μm; Mobile Phase A: Water/5 mM NH₄HCO₃, Mobile Phase B: MeOH, 10%-95% B-3 min (+), 1.50 mL/min, ES, m/z]: T_{R}=2.41 min; [M+H]⁺: 745.

### (1R,2S,3R,5R)-3-(4-Amino-5-(1-methyl-1H-pyrazol-4-yl)-7H-pyrrolo[2,3-d]pyrimidin-7-yl)-5-(((3-(phenethylamino)propyl) amino) methyl) cyclopentane-1,2-diol (478)

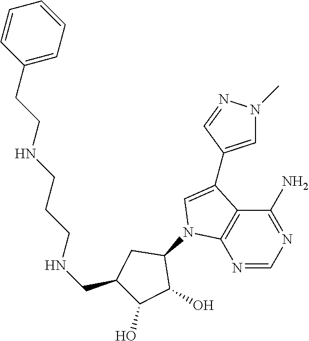 [SMILES: Cn1cc(-c2cn([C@@H]3C[C@H](CNCCCNCCc4ccccc4)[C@@H](O)[C@H]3O)c3ncnc(N)c23)cn1]

To tert-butyl-N-[3-({[(3αR,4R,6R,6αS)-6-[4-amino-5-(1-methylpyrazol-4-yl) pyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate 172a (50 mg, 0.07 mmol, 1.0 eq) was added TFA (1.0 mL) at room temperature and the reaction mixture was stirred at 50° C. for 1 h. The resulting mixture was concentrated under reduced pressure and residue was basified to pH 10 with NH₃.H₂O. The crude product was purified by Prep-HPLC [Column: YMC-Actus Triart C₁₈ ExRS, 30*150 mm, 5 μm; Mobile Phase A: Water (10 mmol/L NH₄HCO₃+0.1% NH₃.H₂O), Mobile Phase B: ACN; Flow rate: 30 ml/min; Gradient: 22% B to 53% B in 7 min, 53% B; Wave Length: 220 nm]: to afford (1R,2S,3R,5R)-3-[4-amino-5-(1-methylpyrazol-4-yl) pyrrolo[2,3-d]pyrimidin-7-yl]-5-[{{3-[(2-phenylethyl) amino]propyl}amino) methyl]cyclopentane-1,2-diol 478 (12 mg, 35%) as an off-white solid. ¹H NMR (400 MHZ, DMSO-d₆) δ8.09 (s, 1H), 7.81 (s, 1H), 7.54 (s, 1H), 7.31 (s, 1H), 7.29-7.11 (m, 5H), 6.12 (br s, 2H), 4.99-4.81 (m, 1H), 4.80-4.61 (m, 1H), 4.33-4.14 (m, 1H), 3.89 (s, 3H), 3.84-3.76 (m, 1H), 2.81-2.54 (m, 10H), 2.26-2.14 (m, 1H), 2.11-1.99 (m, 1H), 1.68-1.45 (m, 3H). LCMS [conditions ZORBAX SB-Aq, 50*4.6 mm, 1.8 μm, Mobile Phase A: Water/0.02% TFA, Mobile Phase B: Acetonitrile, 5%-95% B-3 min, 1.50 mL/min, ES, m/z]: T_{R}=1.07 min; [M+H]⁺: 505. CHIRAL SFC [Column: CHIRALPACK ID-3 50*3.0 mm, 3.0 μm. 50 degree, Solvent B: MeOH (0.2% MSA), Solvent A: CO₂, 10% to 50% in 2.0 min, hold 1.0 min at 50%]: T_{R}=2.19 min, single peak.

### Synthesis of (1R,2S,3R,5R)-3-(4-amino-5-(tetrahydro-2H-pyran-4-yl)-7H-pyrrolo[2,3-d]pyrimidin-7-yl)-5-(((3-(phenethylamino)propyl) amino) methyl) cyclopentane-1,2-diol (479)

### tert-Butyl-(3-((((3αR,4R,6R,6αS)-6-(4-amino-5-(3,6-dihydro-2H-pyran-4-yl)-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(phenethyl)carbamate (138a)

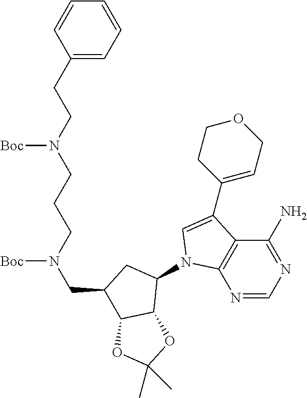 [SMILES: CC(C)(C)OC(=O)N(CCCN(C[C@H]1C[C@@H](n2cc(C3=CCOCC3)c3c(N)ncnc32)[C@@H]2OC(C)(C)O[C@H]12)C(=O)OC(C)(C)C)CCc1ccccc1]

To a stirred mixture of tert-butyl-N-[3-({[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 108a (90 mg, 0.12 mmol, 1.0 eq) and 2-(3,6-dihydro-2H-pyran-4-yl)-4,4,5,5-tetramethyl-1,3,2-dioxaborolane (38 mg, 0.18 mmol, 1.5 eq) in 1,4-dioxane (1.0 mL) and H₂O (0.1 mL) was added Pd (dtbpf) Cl₂ (4.0 mg, 0.006 mmol, 0.05 eq) and K₃PO₄ (77 mg, 0.36 mmol, 3.0 eq) and the reaction mixture was stirred at 80° C. for 1 h, under N₂. The resulting mixture was concentrated under reduced pressure and was disolved in AcOEt (60 mL), washed with saturated brine, dried over anhydrous Na₂SO₄, filtered and the filtrate was concentrated under reduced pressure. The crude residue was purified by Prep-TLC(50:50 tetrahydrofuran: petroleum ether) to afford tert-butyl-N-[3-({[(3αR,4R,6R,6αS)-6-[4-amino-5-(3,6-dihydro-2H-pyran-4-yl) pyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate 138a (80 mg, 89%) as an off-white solid. LCMS [conditions: XBridge C18, 50*3.0 mm, 3.5 μm, Mobile Phase A: Water/5 mM NH₄HCO₃, Mobile Phase B: Methanol, 10%-95% B-2 min (+).Icm, 1.50 mL/min]: T_{R}=1.61 min; [M+H]⁺: 747.

### tert-Butyl-(3-((((3αR,4R,6R,6αS)-6-(4-amino-5-(tetrahydro-2H-pyran-4-yl)-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(phenethyl)carbamate (139a)

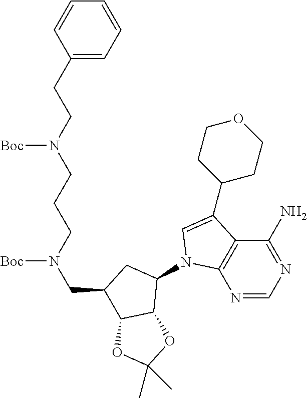 [SMILES: CC(C)(C)OC(=O)N(CCCN(C[C@H]1C[C@@H](n2cc(C3CCOCC3)c3c(N)ncnc32)[C@@H]2OC(C)(C)O[C@H]12)C(=O)OC(C)(C)C)CCc1ccccc1]

To a solution of tert-butyl-N-[3-({[(3αR,4R,6R,6αS)-6-[4-amino-5-(3,6-dihydro-2H-pyran-4-yl) pyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate 138a (70 mg, 0.09 mmol, 1.0 eq) in THF (1.0 mL) was added 10% Pd/C(2.0 mg) in a pressure tank and the reaction mixture was hydrogenated at room temperature under 20 psi of hydrogen pressure for 12 h. The resulting mixture filtered through a Celite pad and concentrated under reduced pressure to afford the crude product tert-butyl-N-[3-({[(3αR,4R,6R,6αS)-6-[4-amino-5-(oxan-4-yl) pyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate 139a (65 mg, 93%) was used in the next step without further purification. LCMS [conditions: XBridge C18, 50*3.0 mm, 3.5 μm, Mobile Phase A: Water/0.05% Ammonia, Mobile Phase B: Acetonitrile, 5%-95% B-5 min (+).Icm, 1.20 mL/min]: T_{R}=3.47 min; [M+H]⁺: 749.

### (1R,2S,3R,5R)-3-(4-Amino-5-(tetrahydro-2H-pyran-4-yl)-7H-pyrrolo[2,3-d]pyrimidin-7-yl)-5-(((3-(phenethylamino)propyl) amino) methyl) cyclopentane-1,2-diol (479)

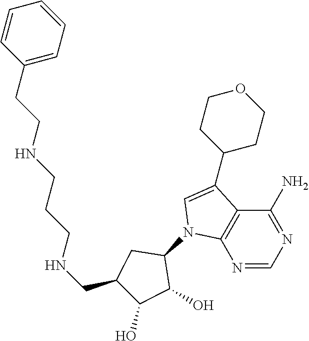 [SMILES: Nc1ncnc2c1c(C1CCOCC1)cn2[C@@H]1C[C@H](CNCCCNCCc2ccccc2)[C@@H](O)[C@H]1O]

To tert-butyl-N-[3-({[(3αR,4R,6R,6αS)-6-[4-amino-5-(oxan-4-yl) pyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate 139a (65 mg, 0.09 mmol, 1.0 eq) was added TFA (1.0 mL) and the reaction mixture was stirred at 50° C. for 1 h. The resulting mixture was concentrated under reduced pressure and was basified to pH >8 with NH₃.H₂O. The crude residue was dissolved in DMF and was purified by Prep-HPLC [Column: YMC-Actus Triart C₁₈ ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 20% B to 55% B in 7 min, 55% B; Wave Length: 220 nm]: to afford (1R,2S,3R,5R)-3-[4-amino-5-(oxan-4-yl) pyrrolo[2,3-d]pyrimidin-7-yl]-5-[({3-[(2-phenylethyl) amino]propyl}amino) methyl]cyclopentane-1,2-diol 479 (32.5 mg, 74%) as an off-white solid. ¹H NMR (400 MHZ, DMSO-d₆+D₂O) 88.00 (s, 1H), 7.29-7.23 (m, 2H), 7.22-7.12 (m, 3H), 7.07 (s, 1H), 4.88-4.77 (m, 1H), 4.19-4.11 (m, 1H), 3.92-3.83 (m, 2H), 3.80-3.75 (m, 1H), 3.54-3.45 (m, 2H), 3.23-3.13 (m, 1H), 2.98-2.54 (m, 10H), 2.17-1.95 (m, 2H), 1.87-1.79 (m, 2H), 1.66-1.41 (m, 5H). LCMS [conditions: ZORBAX SB-Aq, 50*4.6 mm, 1.8 μm, Mobile Phase A: Water/0.05% TFA, Mobile Phase B: Acetonitrile, 5%-95% B-3 min, 1.50 mL/min]: T_{R}=0.98 min; [M+H]⁺: 509. Chiral-SFC [Column: IG 100×4.6 mm 3.0 μm, Solvent B: MeOH (20 mM NH₃), Oven Temperature: 35° C., Total Flow: 3.00 mL/min]: T_{R}=1.70 min; single peak.

### Synthesis of (1R,2S,3R,5R)-3-(4-amino-5-(1-methylpiperidin-4-yl)-7H-pyrrolo[2,3-d]pyrimidin-7-yl)-5-(((3-(phenethylamino)propyl) amino) methyl) cyclopentane-1,2-diol (480)

### tert-Butyl-(3-((((3αR,4R,6R,6αS)-6-(4-amino-5-(1-methyl-1,2,3,6-tetrahydropyridin-4-yl)-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(phenethyl)carbamate (141a)

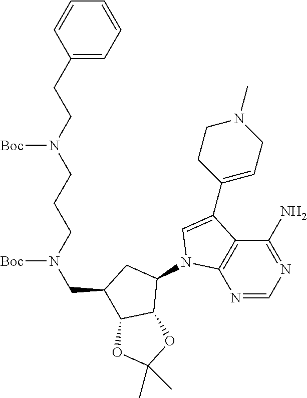 [SMILES: CN1CC=C(c2cn([C@@H]3C[C@H](CN(CCCN(CCc4ccccc4)C(=O)OC(C)(C)C)C(=O)OC(C)(C)C)[C@H]4OC(C)(C)O[C@H]43)c3ncnc(N)c23)CC1]

To a stirred mixture of tert-butyl-N-[3-({[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 108a (110 mg, 0.15 mmol, 1.0 eq) and 1-methyl-4-(4,4,5,5-tetramethyl-1,3,2-dioxaborolan-2-yl)-3,6-dihydro-2H-pyridine (50 mg, 0.22 mmol, 1.5 eq) in 1,4-dioxane (1.5 mL) and H₂O (0.15 mL) were added Pd (dtbpf) Cl₂ (5 mg, 0.007 mmol, 0.05 eq) and K₃PO₄ (95 mg, 0.45 mmol, 3.0 eq) and the reaction mixture was stirred at 80° C. for 1 h, under N₂. The resulting mixture was concentrated under reduced pressure and partitioned between AcOEt and brine. The organic layer was dried over anhydrous Na₂SO₄, filtered and concentrated under reduced pressure. The crude residue was purified by Prep-TLC (50:50 tetrahydrofuran: petroleum ether) to afford tert-butyl-N-[3-({[(3αR,4R,6R,6αS)-6-[4-amino-5-(1-methyl-3,6-dihydro-2H-pyridin-4-yl) pyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate 141a (83 mg, 74%) as an off-white solid. LCMS [conditions: XBridge C18, 50*3.0 mm, 3.5 μm, Mobile Phase A: Water/5 mM NH₄HCO₃, Mobile Phase B: Methanol, 10%-95% B-2.5 min (+).lcm, 1.50 mL/min]: T_{R}=2.03 min; [M+H]⁺: 760

### tert-Butyl-(3-((((3αR,4R,6R,6αS)-6-(4-amino-5-(1-methylpiperidin-4-yl)-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(phenethyl)carbamate (142a)

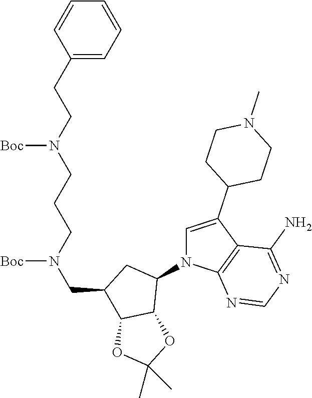 [SMILES: CN1CCC(c2cn([C@@H]3C[C@H](CN(CCCN(CCc4ccccc4)C(=O)OC(C)(C)C)C(=O)OC(C)(C)C)[C@H]4OC(C)(C)O[C@H]43)c3ncnc(N)c23)CC1]

To a solution of tert-butyl-N-[3-({[(3αR,4R,6R,6αS)-6-[4-amino-5-(1-methyl-3,6-dihydro-2H-pyridin-4-yl) pyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate 141a (43 mg, 0.06 mmol, 1.0 eq) in THF (1.0 mL, 0.014 mmol, 0.15 eq) was added 10% Pd/C(1.2 mg,) in a pressure tank and thereaction mixture was hydrogenated at room temperature under 20 atm of hydrogen pressure for 12 h. The resulting mixture was filtered through a Celite pad and concentrated under reduced pressure to afford the crude product tert-butyl-N-[3-({[(3αR,4R,6R,6αS)-6-[4-amino-5-(1-methylpiperidin-4-yl) pyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate 142a (40 mg, 93%) as a yellow solid which was used in the next step without further purification. LCMS [conditions: XBridge C18, 50*3.0 mm, 3.5 μm, Mobile Phase A: Water/0.05% Ammonia, Mobile Phase B: Acetonitrile, 5%-100% B-3 min (+).lcm, 1.20 mL/min]: T_{R}=2.17 min; [M+H]⁺: 762.

### (1R,2S,3R,5R)-3-(4-Amino-5-(1-methylpiperidin-4-yl)-7H-pyrrolo[2,3-d]pyrimidin-7-yl)-5-(((3-(phenethylamino)propyl) amino) methyl) cyclopentane-1,2-diol (480)

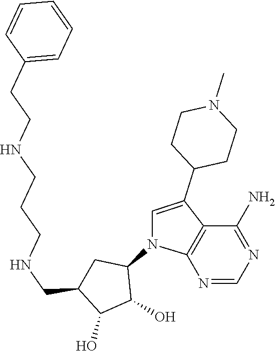 [SMILES: CN1CCC(c2cn([C@@H]3C[C@H](CNCCCNCCc4ccccc4)[C@@H](O)[C@H]3O)c3ncnc(N)c23)CC1]

To tert-butyl-N-[3-({[(3αR,4R,6R,6αS)-6-[4-amino-5-(1-methylpiperidin-4-yl) pyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate (40 mg, 0.04 mmol, 1.0 eq) was added TFA (1.0 mL) and the reaction mixture was stirred at 50° C. for 1 h. The resulting mixture was concentrated under reduced pressure and was basified to pH >8 with NH₃.H₂O. The crude residue was dissolved in DMF and was purified by Prep-HPLC [Column: YMC-Actus Triart C₁₈ ExRS, 30*150 mm, 5 μm; Mobile Phase A: Water (10 mmol/L NH₄HCO₃+0.1% NH₃.H₂O), Mobile Phase B: ACN; Flow rate: 30 ml/min; Gradient: 15% B to 60% B in 7 min, 60% B; Wave Length: 220 nm]: to afford (1R,2S,3R,5R)-3-[4-amino-5-(1-methylpiperidin-4-yl) pyrrolo[2,3-d]pyrimidin-7-yl]-5-[{{3-[(2-phenylethyl) amino]propyl}amino) methyl]cyclopentane-1,2-diol 480 (4.5 mg, 21%) as a white solid. ¹H NMR (400 MHZ, DMSO-d₆+D₂O) 88.01 (s, 1H), 7.30-7.22 (m, 2H), 7.21-7.13 (m, 3H), 7.05 (s, 1H), 6.36 (br s, 2H), 4.89-4.78 (m, 1H), 4.20-4.12 (m, 1H), 3.81-3.74 (m, 1H), 2.99-2.84 (m, 3H), 2.83-2.59 (m, 9H), 2.22-2.16 (m, 3H), 2.14-1.97 (m, 5H), 1.92-1.83 (m, 2H), 1.66-1.43 (m, 5H). LCMS [conditions: ZORBAX SB-Aq, 50*4.6 mm, 1.8 μm, Mobile Phase A: Water/0.05% TFA, Mobile Phase B: Acetonitrile, 5%-95% B-3 min, 1.50 mL/min]: T_{R}=0.85 min; [M+H]⁺: 522. Chiral-SFC [Column: IG 100×4.6 mm 3.0 μm, Solvent B: MeOH (20 mM NH₃), Oven Temperature: 35° C., Total Flow: 3.00 mL/min. Gradient 50% solvent B for 4 mins]: T_{R}=2.09 min; single peak.

### Synthesis of 1-(4-(4-amino-7-((1R,2S,3R,4R)-2,3-dihydroxy-4-(((3-(phenethylamino)propyl) amino) methyl) cyclopentyl)-7H-pyrrolo[2,3-d]pyrimidin-5-yl) piperidin-1-yl)ethan-1-one (481)

### tert-Butyl-(3-((((3αR,4R,6R,6αS)-6-(5-(1-acetyl-1,2,3,6-tetrahydropyridin-4-yl)-4-amino-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(phenethyl)carbamate (143a)

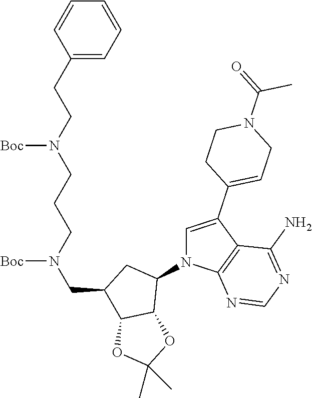 [SMILES: CC(=O)N1CC=C(c2cn([C@@H]3C[C@H](CN(CCCN(CCc4ccccc4)C(=O)OC(C)(C)C)C(=O)OC(C)(C)C)[C@H]4OC(C)(C)O[C@H]43)c3ncnc(N)c23)CC1]

To a stirred mixture of tert-butyl-N-[3-({[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 108a (80 mg, 0.11 mmol, 1.0 eq) and 1-[4-(4,4,5,5-tetramethyl-1,3,2-dioxaborolan-2-yl)-3,6-dihydro-2H-pyridin-1-yl]ethanone (41 mg, 0.16 mmol, 1.5 eq) in dioxane (1.0 mL) and H₂O (0.1 mL) was added Pd (dtbpf) Cl₂ (3.5 mg, 0.005 mmol, 0.05 eq) and K₃PO₄ (90 mg, 0.33 mmol, 3.0 eq) and the reaction mixture was stirred at 80° C. for 1 h, under N₂. The resulting mixture was concentrated under reduced pressure and was partitioned between AcOEt and brine. The orgaic layer was dried over anhydrous Na₂SO₄, filtered and the filtrate was concentrated under reduced pressure. The crude residue was purified by Prep-TLC(50:50 THF: petroleum ether) to afford tert-butyl-N-[3-({[(3αR,4R,6R,6αS)-6-[5-(1-acetyl-3,6-dihydro-2H-pyridin-4-yl)-4-aminopyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate 143a (53 mg, 63%) as an off-white solid. LCMS [conditions: XBridge C18, 50*3.0 mm, 3.5 μm, Mobile Phase A: Water/5 mM NH₄HCO₃, Mobile Phase B: Methanol, 10%-95% B-2 min (+).Icm, 1.00 mL/min]: T_{R}=1.57 min; [M+H]⁺: 788.

### tert-Butyl-(3-((((3αR,4R,6R,6αS)-6-(5-(1-acetylpiperidin-4-yl)-4-amino-7H-pyrrolo[2,3-d]pyrimidin-7-yl)-2,2-dimethyltetrahydro-4H-cyclopenta[d][1,3]dioxol-4-yl)methyl)(tert-butoxycarbonyl) amino)propyl)(phenethyl)carbamate (144a)

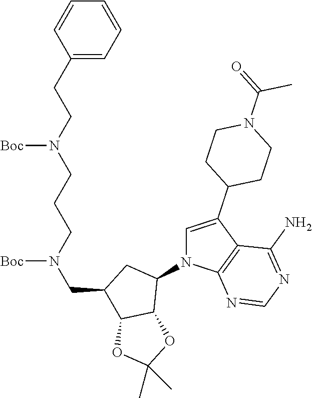 [SMILES: CC(=O)N1CCC(c2cn([C@@H]3C[C@H](CN(CCCN(CCc4ccccc4)C(=O)OC(C)(C)C)C(=O)OC(C)(C)C)[C@H]4OC(C)(C)O[C@H]43)c3ncnc(N)c23)CC1]

To a solution of tert-butyl-N-[3-({[(3αR,4R,6R,6αS)-6-[5-(1-acetyl-3,6-dihydro-2H-pyridin-4-yl)-4-aminopyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N(2-phenylethyl)carbamate 143a (53 mg, 0.07 mmol, 1.0 eq) in THF (1.0 mL) was added Pd/C(1.4 mg, 0.013 mmol, 0.2 eq) in a pressure tank and the reaction mixture was hydrogenated at room temperature under 20 atm of hydrogen pressure for 12 h The resulting mixture was filtered through a Celite pad and concentrated under reduced pressure to afford crude tert-butyl-N-[3-({[(3αR,4R,6R,6αS)-6-[5-(1-acetylpiperidin-4-yl)-4-aminopyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate 144a (50 mL, 94%) which was used in the next step without further purification. LCMS [conditions: XBridge C18, 50*3.0 mm, 3.5 μm, Mobile Phase A: Water/0.05% Ammonia, Mobile Phase B: Acetonitrile, 5%-100% B-3 min (+).lcm, 1.20 mL/min),]: T_{R}=1.90 min; [M+H]⁺: 790.

### 1-(4-(4-Amino-7-((1R,2S,3R,4R)-2,3-dihydroxy-4-(((3-(phenethylamino)propyl) amino) methyl) cyclopentyl)-7H-pyrrolo[2,3-d]pyrimidin-5-yl) piperidin-1-yl)ethan-1-one (481)

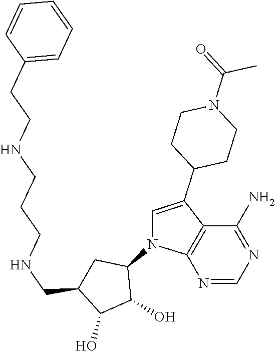 [SMILES: CC(=O)N1CCC(c2cn([C@@H]3C[C@H](CNCCCNCCc4ccccc4)[C@@H](O)[C@H]3O)c3ncnc(N)c23)CC1]

To tert-butyl-N-[3-({[(3αR,4R,6R,6αS)-6-[5-(1-acetylpiperidin-4-yl)-4-aminopyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate (50 mg, 0.06 mmol, 1.0 eq) was added TFA (1.0 mL) and the reaction mixture was stirred at 50° C. for 1 h. The resulting mixture was concentrated under reduced pressure and was basified to pH >8 with NH₃.H₂O. The crude residue was dissolved in DMF and purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 24% B to 49% B in 7 min, 49% B; Wave Length: 220 nm]: to afford 1-(4-{4-amino-7-[(1R,2S,3R,4R)-2,3-dihydroxy-4-[{{3-[(2-phenylethyl) amino]propyl}amino) methyl]cyclopentyl]pyrrolo[2,3-d]pyrimidin-5-yl}piperidin-1-yl)ethanone 481 (20.9 mg, 60%) as a white solid. ¹H NMR (400 MHZ, DMSO-d₆+D₂O) 88.01 (s, 1H), 7.29-7.21 (m, 2H), 7.20-7.11 (m, 3H), 7.06 (s, 1H), 4.88-4.77 (m, 1H), 4.46 (d, J=12.8 Hz, 1H), 4.16-4.08 (m, 1H), 3.89-3.81 (m, 1H), 3.78-3.71 (m, 1H), 3.27-3.17 (m, 2H), 2.79-2.51 (m, 11H), 2.17-2.06 (m, 1H), 2.04-1.95 (m, 1H), 2.01 (s, 3H), 1.95-1.86 (m, 2H), 1.58-1.53 (m, 2H), 1.50-1.24 (m, 3H). LCMS [conditions: ZORBAX SB-Aq, 50*4.6 mm, 1.8 μm, Mobile Phase A: Water/0.05% TFA, Mobile Phase B: Acetonitrile, 5%-95% B-3 min, 1.50 mL/min]: T_{R}=0.98 min; [M+H]⁺: 550. Chiral-SFC [Column: IG 100×4.6 mm 3.0 μm, Solvent B: MeOH (20 mM NH₃), Oven Temperature: 35° C., Total Flow: 3.00 mL/min. Gradient 50% solvent B for 4 mins]: T_{R}=1.31 min, single peak.

### Synthesis of (1R,2S,3R,5R)-3-(4-amino-5-(1-methyl-1,2,3,6-tetrahydropyridin-4-yl)-7H-pyrrolo[2,3-d]pyrimidin-7-yl)-5-(((3-(phenethylamino)propyl) amino) methyl) cyclopentane-1,2-diol (482)

### (1R,2S,3R,5R)-3-(4-Amino-5-(1-methyl-1,2,3,6-tetrahydropyridin-4-yl)-7H-pyrrolo[2,3-d]pyrimidin-7-yl)-5-(((3-(phenethylamino)propyl) amino) methyl) cyclopentane-1.2-diol (482)

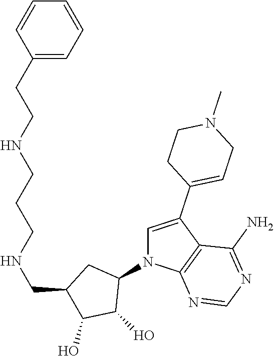 [SMILES: CN1CC=C(c2cn([C@@H]3C[C@H](CNCCCNCCc4ccccc4)[C@@H](O)[C@H]3O)c3ncnc(N)c23)CC1]

To tert-butyl-N-[3-({[(3αR,4R,6R,6αS)-6-[4-amino-5-(1-methyl-3,6-dihydro-2H-pyridin-4-yl) pyrrolo[2,3-d]pyrimidin-7-yl]-2,2-dimethyl-tetrahydro-3αH-cyclopenta[d][1,3]dioxol-4-yl]methyl}(tert-butoxycarbonyl) amino)propyl]-N-(2-phenylethyl)carbamate 141a (40 mg, 0.05 mmol, 1.0 eq) was added TFA (1.0 mL) and the reaction mixture was stirred at 50° C. for 1 h. The resulting mixture was concentrated under reduced pressure and was basified to pH >8 with NH₃.H₂O. The crude residue was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 ml/min; Gradient: 20% B to 60% B in 7 min, 60% B; Wave Length: 220 nm]: to afford (1R,2S,3R,5R)-3-[4-amino-5-(1-methyl-3,6-dihydro-2H-pyridin-4-yl) pyrrolo[2,3-d]pyrimidin-7-yl]-5-[{{3-[(2-phenylethyl) amino]propyl}amino) methyl]cyclopentane-1,2-diol 482 (6.6 mg, 24%) as an off-white solid. ¹H NMR (400 MHZ, DMSO-d₆+D₂O) 88.07 (s, 1H), 7.36 (s, 1H), 7.31-7.21 (m, 2H), 7.20-7.12 (m, 3H), 5.67 (s, 1H), 4.93-4.82 (m, 1H), 4.21-4.13 (m, 1H), 3.80-3.73 (m, 1H), 3.05-2.99 (m, 2H), 2.74-2.54 (m, 12H), 2.49-2.45 (m, 2H), 2.31-2.26 (m, 3H), 2.21-2.10 (m, 1H), 2.08-2.03 (m, 1H), 1.61-1.54 (m, 2H), 1.53-1.42 (m, 1H). LCMS [conditions: ZORBAX SB-Aq, 50*4.6 mm, 1.8 μm, Mobile Phase A: Water/0.05% TFA, Mobile Phase B: Acetonitrile, 5%-95% B-3 min, 1.50 mL/min]: T_{R}=0.85 min; [M+H]⁺: 520. Chiral-SFC [Column: IG 100×4.6 mm 3.0 μm, Solvent B: MeOH (20 mM NH₃), Oven Temperature: 35° C., Total Flow: 3.00 mL/min. Gradient 50% solvent B for 4 mins]: T_{R}=1.65 min, single peak.

### Synthesis of 4-amino-7-((1R,2S,3R,4R)-2,3-dihydroxy-4-(((3-(phenethylamino)propyl) amino) methyl) cyclopentyl)-7H-pyrrolo[2,3-d]pyrimidine-5-carbonitrile (483)

### Synthesis of tert-butyl-N-[3-({[(3αR,4R,6R,6αS)-6-{4-amino-5-cyanopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate (152a)

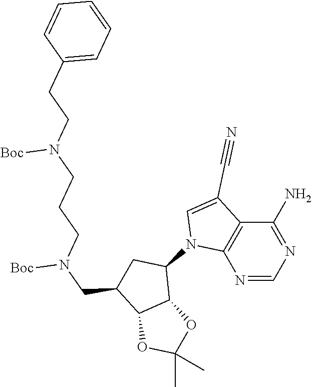 [SMILES: CC(C)(C)OC(=O)N(CCCN(C[C@H]1C[C@@H](n2cc(C#N)c3c(N)ncnc32)[C@@H]2OC(C)(C)O[C@H]12)C(=O)OC(C)(C)C)CCc1ccccc1]

To a stirred mixture of tert-butyl-N-[3-({[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 108a (100 mg, 0.13 mmol, 1.0 eq), Zn (CN) 2 (47 mg, 0.40 mmol, 3.0 eq), dppf (0.03 mmol, 0.20 eq) in NMP (2.0 mL) was added Pd₂ (dba)₃ (12 mg, 0.013 mmol, 0.1 eq) at room temperature under nitrogen and the reaction mixture was irradiated under microwave radiation for 0.5 h at 140° C. The resulting mixture was allowed to cool to room temperature and was concentrated under vacuum. The crude residue was purified by silica gel column chromatography (90:10 CH₂Cl₂: MeOH) to afford tert-butyl-N-[3-({[(3αR,4R,6R,6αS)-6-{4-amino-5-cyanopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 152a (80 mg, 86%) as a yellow solid. LCMS [Xbridge Shield RP18, 50*3.0 mm; Mobile Phase A: Water/0.05% Ammonia, Mobile Phase B: Acetonitrile, 5%-100% B-2 min (+), 1.20 mL/min, ES, m/z]: T_{R}=1.36 min; [M+H]⁺: 690.

### 4-Amino-7-((1R,2S,3R,4R)-2,3-dihydroxy-4-(((3-(phenethylamino)propyl) amino) methyl) cyclopentyl)-7H-pyrrolo[2,3-d]pyrimidine-5-carbonitrile (483)

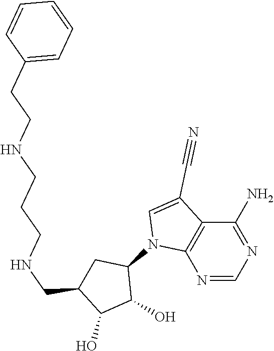 [SMILES: N#Cc1cn([C@@H]2C[C@H](CNCCCNCCc3ccccc3)[C@@H](O)[C@H]2O)c2ncnc(N)c12]

To tert-butyl-N-[3-({[(3αR,4R,6R,6αS)-6-{4-amino-5-cyanopyrrolo[2,3-d]pyrimidin-7-yl}-2,2-dimethyl-tetrahydro-3αH-cyclopenta[d][1,3]dioxol-4-yl]methyl}(tert-butoxycarbonyl) amino)propyl]-N-(2-phenylethyl)carbamate 152a (80 mg, 0.12 mmol, 1.0 eq) was added TFA (2.0 mL) and the reaction mixture was stirred for 1 h at room temperature. The resulting mixture was concentrated under reduced pressure and was basified to pH >8 with NH₃·H₂O and the crude residue was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 ml/min; Gradient: 20% B to 60% B in 7 min, 60% B; Wave Length: 220 nm]: to afford 4-amino-7-((1R,2S,3R,4R)-2,3-dihydroxy-4-(((3-(phenethylamino)propyl) amino) methyl) cyclopentyl)-7H-pyrrolo[2,3-d]pyrimidine-5-carbonitrile 483 (10.2 mg, 20%) as a white solid. ¹H NMR (300 MHZ, DMSO-d₆) δ 8.38 (s, 1H), 8.21 (s, 1H), 7.31-7.23 (m, 2H), 7.22-7.10 (m, 3H), 6.79 (br s, 2H), 4.90-4.85 (m, 2H), 4.25-4.22 (m, 1H), 3.77-3.73 (m, 1H), 2.81-2.50 (m, 9H), 2.31-2.19 (m, 1H), 2.08-1.97 (m, 1H), 1.55-1.49 (m, 3H). LCMS [conditions: ZORBAX SB-Aq, 50*4.6 mm, 1.8 μm, Mobile Phase A: Water/0.05% TFA, Mobile Phase B: Acetonitrile, 5%-95% B-3 min, 1.50 mL/min]: T_{R}=1.03 min; [M+H]⁺: 450. Chiral-SFC [Column: Lux-4 100×4.6 mm 3.0 μm, Solvent B: MeOH (20 mM NH₃), Oven Temperature: 35° C., Total Flow: 3.00 mL/min. Gradient 30% solvent B for 4 mins]: T_{R}=2.66 min, single peak.

### Synthesis of (1R,2S,3R,5R)-3-(4-Amino-5-bromo-7H-pyrrolo[2,3-d]pyrimidin-7-yl)-5-((3-((phenethylamino) methyl) azetidin-1-yl)methyl) cyclopentane-1,2-diol (484)

### Synthesis of tert-butyl-(azetidin-3-ylmethyl)(phenethyl)carbamate (114a) Benzyl 3-(((tert-butoxycarbonyl) amino) methyl) azetidine-1-carboxylate (118a)

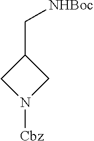 [SMILES: CC(C)(C)OC(=O)NCC1CN(C(=O)OCc2ccccc2)C1]

To a stirred solution of tert-butyl-N-(azetidin-3-ylmethyl)carbamate (1.00 g, 5.40 mmol, 1.0 eq) and TEA (1.09 g, 10.7 mmol, 2.0 eq) in DCM (10.0 mL) was added, dropwise, Cbz-Cl (1.01 g, 5.90 mmol, 1.1 eq) in DCM (5.0 mL), and the reaction mixture was stirred at room temperature for 1 h. The resulting mixture was extracted with DCM (3×20 mL) and the combined organic layers were washed with saturated ammonium chloride solution, brine, dried over anhydrous Na₂SO₄ and filtered. The filtrate was concentrated under reduced pressure and the crude residue was purified by silica gel column chromatography (60:40 AcOEt: petroleum ether) to afford benzyl 3-{[(tert-butoxycarbonyl) amino]methyl}azetidine-1-carboxylate 118a (1.50 g, 87%) as a colorless oil.

### Benzyl 3-(aminomethyl) azetidine-1-carboxylate (119a)

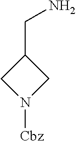 [SMILES: NCC1CN(C(=O)OCc2ccccc2)C1]

To benzyl 3-{[(tert-butoxycarbonyl) amino]methyl}azetidine-1-carboxylate 118a (1.50 g, 4.70 mmol, 1.0 eq) in DCM (20 mL) was aded TFA (1.07 g, 9.40 mmol, 2.0 eq), at room temperature, and the reaction mixture was stirred for 1 h. The resulting mixture was concentrated under reduced pressure to afford benzyl 3-(aminomethyl) azetidine-1-carboxylate 119a (1.10 g, 96%) which was used in the next step directly without further purification.

### Benzyl 3-((phenethylamino) methyl) azetidine-1-carboxylate (120a)

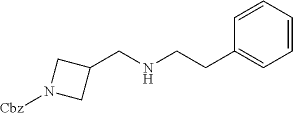 [SMILES: O=C(OCc1ccccc1)N1CC(CNCCc2ccccc2)C1]

To a stirred solution of benzyl 3-(aminomethyl) azetidine-1-carboxylate 119a (1.10 g, 2.50 mmol, 1.0 eq) and NaBH (OAc)₃ (0.79 g, 3.70 mmol, 1.5 eq) in DCM (10 ml) was added phenylacetaldehyde (0.30 g, 2.50 mmol, 1.0 eq), dropwise, at 0° C. and the reaction mixture was stirred for 0.5 h at room temperature. The resulting mixture was diluted with dichloromethane (60 mL) and washed with brine, dried over anhydrous Na₂SO₄ and filtered. The filtrate was concentrated under reduced pressure and the crude residue was purified by reverse flash chromatography [column, C₁₈ silica gel; mobile phase, CH₃CN in water, 20% to 45% gradient in 12 min; detector, UV 220 nm]: to afford benzyl 3-{[(2-phenylethyl) amino]methyl}azetidine-1-carboxylate 120a (420 mg, 52%) as a colorless oil.

### Benzyl 3-(((tert-butoxycarbonyl)(phenethyl) amino) methyl) azetidine-1-carboxylate (121a)

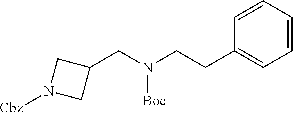 [SMILES: CC(C)(C)OC(=O)N(CCc1ccccc1)CC1CN(C(=O)OCc2ccccc2)C1]

To benzyl 3-{[(2-phenylethyl) amino]methyl}azetidine-1-carboxylate 120a (0.32 g, 0.70 mmol, 1.0 eq) in MeOH (3.0 mL) was added Boc₂O (0.19 g, 0.90 mmol, 1.2 eq) and the reaction mixture was stirred for 1 h at room temperature. The resulting mixture was evaporated and the crude residue was purified by reverse flash chromatography [column, C₁₈ silica gel; mobile phase, MeOH in water, 30% to 80% gradient in 10 min; detector, UV 220 nm]: to afford benzyl 3-{[(tert-butoxycarbonyl)(2-phenylethyl) amino]methyl}azetidine-1-carboxylate 121a (230 mg, 73%) as a colorless oil.

### tert-Butyl(azetidin-3-ylmethyl)(phenethyl)carbamate (114a)

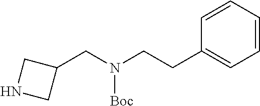 [SMILES: CC(C)(C)OC(=O)N(CCc1ccccc1)CC1CNC1]

To a solution of benzyl 3-{[(tert-butoxycarbonyl)(2-phenylethyl) amino]methyl}azetidine-1-carboxylate 121a (0.23 g, 0.50 mmol, 1.0 eq) in MeOH (3.0 mL) was added Pd/C(10%, 6 mg) in a pressure tank. The reaction mixture was hydrogenated at room temperature under 30 psi of hydrogen pressure for 1 h. The resulting mixture was filtered through a Celite pad and the filtrate was concentrated under reduced pressure to afford crude tert-butyl-N-(azetidin-3-ylmethyl)-N-(2-phenylethyl)carbamate 114a (140 mg, 89%) which was used in the next step directly without further purification. LCMS [conditions: Xbridge Shield RP18, 50*3.0 mm, 3.5 μm, Mobile Phase A: Water/0.05% Ammonia, Phase B: Acetonitrile, 5%-100% B-2 min, 1.50 mL/min]: T_{R}=1.18 min; [M+H]⁺: 291.

### tert-Butyl-((1-(((3αR,4R,6R,6αS)-6-(4-chloro-7H-pyrrolo[2,3-d]pyrimidin-7-yl)-2,2-dimethyltetrahydro-4H-cyclopenta[d][1,3]dioxol-4-yl)methyl) azetidin-3-yl)methyl)(phenethyl)carbamate (115a)

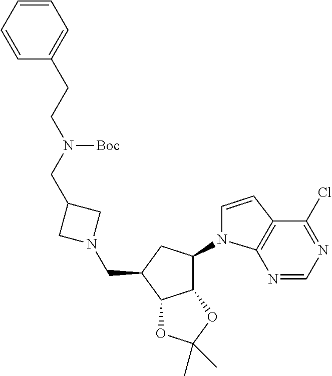 [SMILES: CC(C)(C)OC(=O)N(CCc1ccccc1)CC1CN(C[C@H]2C[C@@H](n3ccc4c(Cl)ncnc43)[C@@H]3OC(C)(C)O[C@H]23)C1]

To tert-butyl-N-(azetidin-3-ylmethyl)-N-(2-phenylethyl)carbamate 114a (98 mg, 0.34 mmol, 1.0 eq), in DCM (2.0 mL) was added AcOH (0.21 g, 3.40 mmol, 10.0 eq) at room temperature and the resulting mixture was stirred for 30 min at room temperature and then (3αR,4S,6R,6αS)-6-{4-chloropyrrolo[2,3-d]pyrimidin-7-yl}-2,2-dimethyl-tetrahydro-3αH-cyclopenta[d][1,3]dioxole-4-carbaldehyde 77 (WO2020033288)(0.15 g, 0.40 mmol, 1.2 eq) in DCM (2.0 mL) was added dropwise at room temperature. The reaction mixture was stirred for 1 h at room temperature and concentrated under reduced pressure. The crude residue was dissolved in methanol and purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 25% B to 50% B in 7 min, 50% B; Wave Length: 220 nm]: to afford tert-butyl-N-[(1-{[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}azetidin-3-yl)methyl]-N-(2-phenylethyl)carbamate 115a (130 mg, 65%) as a white solid. LCMS [conditions: Xbridge Shield RP18, 50*3.0 mm, 3.5 μm, Mobile Phase A: Water/0.05% Ammonia, Phase B: Acetonitrile, 5%-95% B-5 min, 1.50 mL/min]: T_{R}=3.57 min; [M+H]⁺: 596.

### tert-Butyl-((1-(((3αR,4R,6R,6αS)-6-(5-bromo-4-chloro-7H-pyrrolo[2,3-d]pyrimidin-7-yl)-2,2-dimethyltetrahydro-4H-cyclopenta[d][1,3]dioxol-4-yl)methyl) azetidin-3-yl)methyl)(phenethyl)carbamate (116a)

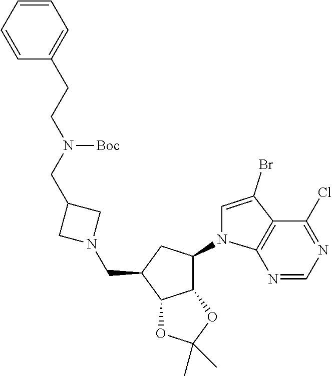 [SMILES: CC(C)(C)OC(=O)N(CCc1ccccc1)CC1CN(C[C@H]2C[C@@H](n3cc(Br)c4c(Cl)ncnc43)[C@@H]3OC(C)(C)O[C@H]23)C1]

To tert-butyl-N-[(1-{[(3αR,4R,6R,6αS)-6-{4-chloropyrrolo[2,3-d]pyrimidin-7-yl}-2,2-dimethyl-tetrahydro-3αH-cyclopenta[d][1,3]dioxol-4-yl]methyl}azetidin-3-yl)methyl]-N(2-phenylethyl)carbamate 115a (0.11 g, 0.20 mmol, 1.0 eq) in DMF (2.0 mL) was added Br2 (33 mg, 0.20 mmol, 1.1 eq) in DMF (1.0 mL), dropwise, at 0° C. and the reaction mixture was stirred for 4 h at room temperature. The resulting mixture was quenched by the addition of saturated sodium thiosulfate solution and was extracted with AcOEt (3×10 mL). The combined organic layers were washed with saturated sodium bicarbonate solution, brine and dried over anhydrous sodium sulfate. The solute was filtered, concentrated under reduced pressure and the crude residue was purified by Prep-TLC(95:5 DCM: MeOH) to afford tert-butyl-N-[(1-{[(3αR,4R,6R,6αS)-6-{5-bromo-4-chloropyrrolo[2,3-d]pyrimidin-7-yl}-2,2-dimethyl-tetrahydro-3αH-cyclopenta[d][1,3]dioxol-4-yl]methyl}azetidin-3-yl)methyl]-N-(2-phenylethyl)carbamate 116a (94 mg, 76%) as a white solid. LCMS [conditions: ZORBAX SB-Aq, 50*4.6 mm, 1.8 μm, Mobile Phase A: Water/0.05% TFA, Phase B: Acetonitrile, 5%-95% B-3 min, 1.50 mL/min]: T_{R}=1.12 min; [M+H]⁺: 674, 676.

### tert-Butyl-((1-(((3αR,4R,6R,6αS)-6-(4-amino-5-bromo-7H-pyrrolo[2,3-d]pyrimidin-7-yl)-2,2-dimethyltetrahydro-4H-cyclopenta[d][1,3]dioxol-4-yl)methyl) azetidin-3-yl)methyl)(phenethyl)carbamate. (117a)

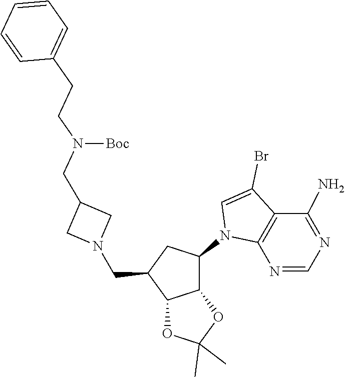 [SMILES: CC(C)(C)OC(=O)N(CCc1ccccc1)CC1CN(C[C@H]2C[C@@H](n3cc(Br)c4c(N)ncnc43)[C@@H]3OC(C)(C)O[C@H]23)C1]

To tert-butyl-N-[(1-{[(3αR,4R,6R,6αS)-6-{5-bromo-4-chloropyrrolo[2,3-d]pyrimidin-7-yl}-2,2-dimethyl-tetrahydro-3αH-cyclopenta[d][1,3]dioxol-4-yl]methyl}azetidin-3-yl)methyl]-N(2-phenylethyl)carbamate 116a (94 mg, 0.13 mmol, 1.0 eq) was added NH₃ (7M in MeOH, 2.0 mL) at room temperature and the reaction mixture was stirred at 75° C. for 12 h. The resulting mixture was concentrated under reduced pressure to afford the crude product tert-butyl-N-[(1-{[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}azetidin-3-yl)methyl]-N-(2-phenylethyl)carbamate 117a (80 mg) which was used in the next step without further purification. LCMS [Cortecs C_{18+, 50*3.0} mm, 2.7 μm; Mobile Phase A: Water/0.05% TFA, Phase B: Acetonitrile, 2%-100% B-2 min (+), 1.50 mL/min, ES, m/z]: T_{R}=0.88 min; [M+H]⁺: 655, 657.

### (1R,2S,3R,5R)-3-(4-Amino-5-bromo-7H-pyrrolo[2,3-d]pyrimidin-7-yl)-5-((3-((phenethylamino) methyl) azetidin-1-yl) methyl) cyclopentane-1.2-diol (484)

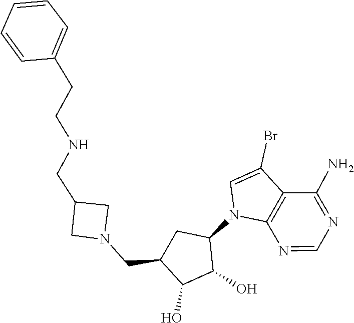 [SMILES: Nc1ncnc2c1c(Br)cn2[C@@H]1C[C@H](CN2CC(CNCCc3ccccc3)C2)[C@@H](O)[C@H]1O]

To tert-butyl-N-[(1-{[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}azetidin-3-yl)methyl]-N(2-phenylethyl)carbamate 117a (80 mg, 0.10 mmol, 1.0 eq) was added TFA (2.0 mL) and the reaction mixture was stirred at 40° C. for 0.5 h. The resulting mixture was concentrated under reduced pressure and was basified to pH >8 with NH₃.H₂O. The crude residue was dissolved in DMF and purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 20% B to 55% B in 7 min, 55% B; Wave Length: 220 nm]: to afford (1R,2S,3R,5R)-3-{4-amino-5-bromopyrrolo[2,3-d]pyrimidin-7-yl}-5-[(3-{[(2-phenylethyl) amino]methyl}azetidin-1-yl)methyl]cyclopentane-1,2-diol 484 (29.9 mg, 53%) as a white solid. ¹H NMR (300 MHZ, DMSO-d₆) δ8.08 (s, 1H), 7.57 (s, 1H), 7.31-7.24 (m, 2H), 7.23-7.12 (m, 3H), 6.67 (br s, 2H), 4.92-4.82 (m, 3H), 4.18-4.10 (m, 1H), 3.73-3.66 (m, 1H), 3.27-3.20 (m, 2H), 2.82-2.60 (m, 8H), 2.52-2.51 (m, 1H), 2.47-2.28 (m, 2H), 2.19-2.07 (m, 1H), 1.87-1.78 (m, 1H), 1.47-1.35 (m, 1H). LCMS [conditions: ZORBAX SB-Aq, 50*4.6 mm, 1.8 μm, Mobile Phase A: Water/0.05% TFA, Mobile Phase B: Acetonitrile, 5%-95% B-3 min, 1.50 mL/min]: T_{R}=0.98 min; [M+H]⁺: 515, 517. Chiral-SFC [Column: Lux-4 100×4.6 mm 3.0 μm, Solvent B: MeOH (20 mM NH₃), Oven Temperature: 35° C., Total Flow: 3.00 mL/min]: T_{R}=1.39 min (major isomer), T_{R}=2.48 min (minor isomer), dr=97.8:2.2.

### Synthesis of (1R,2S,3R,5R)-3-(4-amino-5-vinyl-7H-pyrrolo[2,3-d]pyrimidin-7-yl)-5-((3-(((4-fluorophenethyl) amino) methyl) azetidin-1-yl)methyl) cyclopentane-1,2-diol (485)

### ((3αR,4R,6R,6αS)-6-(5-bromo-4-chloro-7H-pyrrolo[2,3-d]pyrimidin-7-yl)-2,2-dimethyltetrahydro-4H-cyclopenta[d][1,3]dioxol-4-yl) methanol (163a)

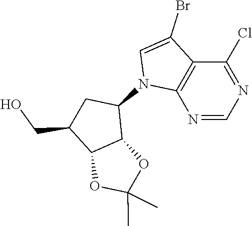 [SMILES: CC1(C)O[C@@H]2[C@@H](CO)C[C@@H](n3cc(Br)c4c(Cl)ncnc43)[C@@H]2O1]

To a solution of methyl-(3αR,4S,6R,6αS)-6-{5-bromo-4-chloropyrrolo[2,3-d]pyrimidin-7-yl}-2,2-dimethyl-tetrahydro-3αH-cyclopenta[d][1,3]dioxole-4-carboxylate 23a (0.50 g, 1.20 mmol, 1.0 eq) in MeOH (5.0 mL) was added NaBH₄ (88 mg, 2.30 mmol, 2.0 eq), at room temperature and the reaction mixture was stirred for 3 h at room temperature. The resulting mixture was quenched with saturated ammonium chloride solution and extracted with AcOEt (3×20 mL). The combined organic layers were washed with brine, dried over anhydrous Na₂SO₄ and filtered. The filtrate was concentrated under reduced pressure and the crude residue was purified by silica gel column chromatography (40:60 AcOEt: petroleum ether) to afford [(3αR,4R,6R,6αS)-6-{5-bromo-4-chloropyrrolo[2,3-d]pyrimidin-7-yl}-2,2-dimethyl-tetrahydro-3αH-cyclopenta[d][1,3]dioxol-4-yl]methanol 163a (0.30 g, 64%) as a yellow solid. LCMS [Xbridge Shield C18, 50*3.0 mm; Mobile Phase A: Water/5 mM NH₄HCO₃, Mobile Phase B: MeOH, 5%-100% B-2.5 min (+)-P, 1.50 mL/min, ES, m/z]: T_{R}=1.35 min; [M+H]⁺: 402, 404.

### (3αR,4S,6R,6αS)-6-(5-Bromo-4-chloro-7H-pyrrolo[2,3-d]pyrimidin-7-yl)-2,2-dimethyltetrahydro-4H-cyclopenta[d][1,3]dioxole-4-carbaldehyde (164a)

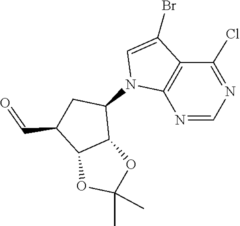 [SMILES: CC1(C)O[C@@H]2[C@H](O1)[C@@H](C=O)C[C@H]2n1cc(Br)c2c(Cl)ncnc21]

To [(3αR, 4R, 6R, 6αS)-6-{5-bromo-4-chloropyrrolo[2,3-d]pyrimidin-7-yl}-2,2-dimethyl-tetrahydro-3αH-cyclopenta[d][1,3]dioxol-4-yl]methanol 163a (0.18 g, 0.50 mmol, 1.0 eq) in DCM (3.0 mL) was added DMP (0.27 g, 0.60 mmol, 1.4 eq) at room temperature and the reaction mixture was stirred for 1 h at room temperature. The crude mixture was purified by silica gel column chromatography (40:60 AcOEt: petroleum ether) to afford (3αR,4S,6R,6αS)-6-{5-bromo-4-chloropyrrolo[2,3-d]pyrimidin-7-yl}-2,2-dimethyl-tetrahydro-3αH-cyclopenta[d][1,3]dioxole-4-carbaldehyde 164a (0.14 g, 78%) as a yellow solid. LCMS [XBridge C18, 50*3.0 mm, 3.5 μm; Mobile Phase A: Water/5 mM NH₄HCO₃, Mobile Phase B: MeOH, 10%-95% B-2 min (+), 1.00 mL/min, ES, m/z]: T_{R}=0.77 min; [M+H]⁺: 400, 402.

### tert-Butyl-((1-(((3αR,4R,6R,6αS)-6-(5-bromo-4-chloro-7H-pyrrolo[2,3-d]pyrimidin-7-yl)-2,2-dimethyltetrahydro-4H-cyclopenta[d][1,3]dioxol-4-yl)methyl) azetidin-3-yl)methyl)(4-fluorophenethyl)carbamate (166a)

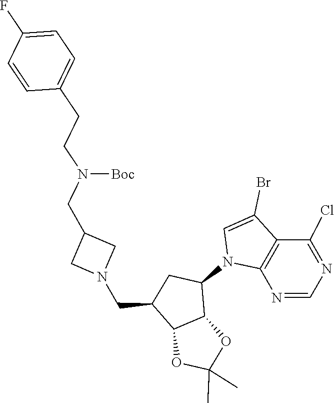 [SMILES: CC(C)(C)OC(=O)N(CCc1ccc(F)cc1)CC1CN(C[C@H]2C[C@@H](n3cc(Br)c4c(Cl)ncnc43)[C@@H]3OC(C)(C)O[C@H]23)C1]

To tert-butyl-N-(azetidin-3-ylmethyl)-N-[2-(4-fluorophenyl)ethyl]carbamate 165a (0.12 g, 0.40 mmol, 1.1 eq) in DCM (2.0 ml) was added NaBH (OAc)₃ (0.15 g, 0.70 mmol, 2.0 eq) and AcOH (0.21 mg, 3.50 mmol, 10 eq) at room temperature and the reaction mixture was stirred for 30 min at room temperature. To the resulting mixture was added (3αR,4S,6R,6αS)-6-{5-bromo-4-chloropyrrolo[2,3-d]pyrimidin-7-yl}-2,2-dimethyl-tetrahydro-3αH-cyclopenta[d][1,3]dioxole-4-carbaldehyde 164a (0.14 g, 0.35 mmol, 1.0 eq) in DCM (1.0 mL) and the reaction mixture was stirred for 1 h at room temperature. The resulting mixture was concentrated under reduced pressure and the crude product was purified by Prep-HPLC [Column: Xtimate C18 50×250 mm, 10 μm; Mobile Phase A: Water (0.05% NH₃·H₂O), Mobile Phase B: ACN; Flow rate: 90 mL/min; Gradient: 90% B in 16 min; Wave Length: 220 nm]: to afford tert-butyl-N-[(1-{[(3αR,4R,6R,6αS)-6-{5-bromo-4-chloropyrrolo[2,3-d]pyrimidin-7-yl}-2,2-dimethyl-tetrahydro-3αH-cyclopenta[d][1,3]dioxol-4-yl]methyl}azetidin-3-yl)methyl]-N-[2-(4-fluorophenyl)ethyl]carbamate 166a (160 mg, 66%) as a white solid. LCMS [XBridge C18, 50*3.0 mm, 3.5 μm; Mobile Phase A: Water/5 mM NH₄HCO₃, Mobile Phase B: MeOH, 10%-95% B-5 min (+), 1.50 mL/min, ES, m/z]: T_{R}=4.29 min; [M+H]⁺: 692, 694.

### tert-Butyl-((1-(((3αR,4R,6R,6αS)-6-(4-amino-5-bromo-7H-pyrrolo[2,3-d]pyrimidin-7-yl)-2,2-dimethyltetrahydro-4H-cyclopenta[d][1,3]dioxol-4-yl)methyl) azetidin-3-yl)methyl)(4-fluorophenethyl)carbamate (167a)

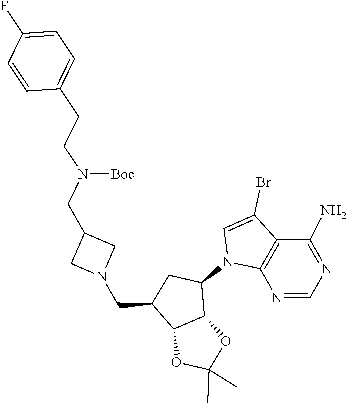 [SMILES: CC(C)(C)OC(=O)N(CCc1ccc(F)cc1)CC1CN(C[C@H]2C[C@@H](n3cc(Br)c4c(N)ncnc43)[C@@H]3OC(C)(C)O[C@H]23)C1]

To tert-butyl-N-[(1-{[(3αR,4R,6R,6αS)-6-{5-bromo-4-chloropyrrolo[2,3-d]pyrimidin-7-yl}-2,2-dimethyl-tetrahydro-3αH-cyclopenta[d][1,3]dioxol-4-yl]methyl}azetidin-3-yl) methyl]-N-[2-(4-fluorophenyl)ethyl]carbamate 166a (0.16 g, 0.20 mmol, 1.0 eq) was added NH₃ (7M in MeOH, 3.0 mL) at room temperature and the reaction mixture was stirred at 75° C. for 12 h. The resulting mixture was concentrated under reduced pressure and the crude sample was purified by silica gel column chromatography (50:50 AcOEt: petroleum ether) to afford tert-butyl-N-[(1-{[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}azetidin-3-yl)methyl]-N-[2-(4-fluorophenyl)ethyl]carbamate 167a (0.14 g, 87%) as an off-white solid. LCMS [XBridge C18, 50*3.0 mm, 3.5 μm; Mobile Phase A: Water/5 mM NH₄HCO₃+0.05% Ammonia, Mobile Phase B: Acetonitrile/5% water, 5%-100% B-2 min (+).lcm, 1.20 mL/min, ES, m/z]: T_{R}=1.56 min; [M+H]⁺: 673, 675.

### (1R,2S,3R,5R)-3-(4-Amino-5-bromo-7H-pyrrolo[2,3-d]pyrimidin-7-yl)-5-((3-(((4-fluorophenethyl) amino) methyl) azetidin-1-yl)methyl) cyclopentane-1,2-diol (168a)

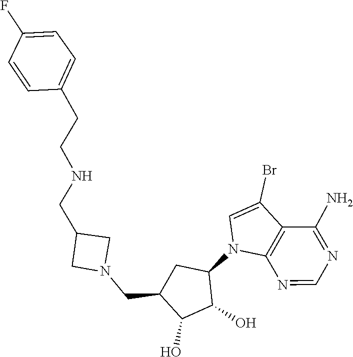 [SMILES: Nc1ncnc2c1c(Br)cn2[C@@H]1C[C@H](CN2CC(CNCCc3ccc(F)cc3)C2)[C@@H](O)[C@H]1O]

To tert-butyl-N-[(1-{[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}azetidin-3-yl)methyl]-N-[2-(4-fluorophenyl)ethyl]carbamate 167a (0.14 g, 0.20 mmol, 1.0 eq) was added TFA (1.0 mL) and the reaction mixture was stirred at 50° C. for 0.5 h. The resulting mixture was concentrated under reduced pressure and basified to pH >8 with NH₃.H₂O. The crude residue was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 20% B to 55% B in 8 min, 55% B; Wave Length: 220 nm]: to afford (1R,2S,3R,5R)-3-{4-amino-5-bromopyrrolo[2,3-d]pyrimidin-7-yl}-5-{[3-({[2-(4-fluorophenyl)ethyl]amino}methyl) azetidin-1-yl]methyl}cyclopentane-1,2-diol 168a (73 mg, 68%) as an off-white solid. LCMS [L-column3 ODS, 50*3.0 mm, 3.0 μm; Mobile Phase A: Water/5 mM NH₄HCO₃+0.05% Ammonia, Mobile Phase B: Acetonitrile/5% water, 5%-100% B-2.5 min-1.2 mL-P.Icm, 1.50 mL/min, ES, m/z]: T_{R}=1.21 min; [M+H]⁺: 533, 535.

### (1R,2S,3R,5R)-3-(4-Amino-5-vinyl-7H-pyrrolo[2,3-d]pyrimidin-7-yl)-5-((3-(((4-fluorophenethyl) amino) methyl) azetidin-1-yl)methyl) cyclopentane-1,2-diol (485)

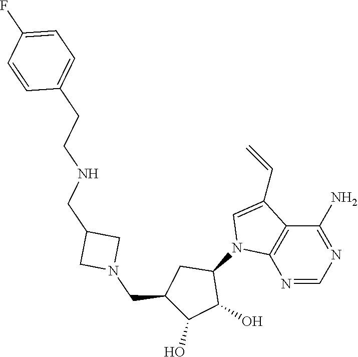 [SMILES: C=Cc1cn([C@@H]2C[C@H](CN3CC(CNCCc4ccc(F)cc4)C3)[C@@H](O)[C@H]2O)c2ncnc(N)c12]

To a stirred mixture of (1R,2S,3R,5R)-3-{4-amino-5-bromopyrrolo[2,3-d]pyrimidin-7-yl}-5-{[3-({[2-(4-fluorophenyl)ethyl]amino}methyl) azetidin-1-yl]methyl}cyclopentane-1,2-diol 168a (73 mg, 0.14 mmol, 1.0 eq) and 2-ethenyl-4,4,5,5-tetramethyl-1,3,2-dioxaborolane (32 mg, 0.21 mmol, 1.5 eq) in 1,4-dioxane (1.0 mL) and H₂O (0.1 mL) were added Pd (dtbpf) Cl₂ (5.0 mg, 0.007 mmol, 0.05 eq) and K₃PO₄ (87 mg, 0.41 mmol, 3.0 eq) and the reaction mixture was stirred at 80° C. for 0.5 h, under N₂. The resulting mixture was concentrated under reduced pressure and partitioned between AcOEt and brine. The organic layer was dried over anhydrous Na₂SO₄, filtered and the filtrate was concentrated under reduced pressure. The crude residue was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 22% B to 50% B in 9 min, 50% B; Wave Length: 220 nm]: to afford (1R,2S,3R,5R)-3-{4-amino-5-ethenylpyrrolo[2,3-d]pyrimidin-7-yl}-5-{[3-({[2-(4-fluorophenyl)ethyl]amino}methyl) azetidin-1-yl]methyl}cyclopentane-1,2-diol 485 (28.7 mg, 44%) as a white solid. ¹H NMR (300 MHZ, DMSO-d₆) δ 8.03 (s, 1H), 7.60 (s, 1H), 7.30-7.19 (m, 2H), 7.17-7.01 (m, 3H), 6.61 (br s, 2H), 5.55 (d, J=17.1 Hz, 1H), 5.07 (d, J=12.6 Hz, 1H), 4.96-4.70 (m, 2H), 4.67-4.29 (m, 1H), 4.23-4.08 (m, 1H), 3.77-3.64 (m, 1H), 3.33-3.18 (m, 2H), 2.82-2.72 (m, 2H), 2.71-2.60 (m, 6H), 2.47-2.24 (m, 2H), 2.20-2.07 (m, 1H), 1.91-1.76 (m, 1H), 1.51-1.34 (m, 1H). LCMS [conditions: ZORBAX SB-Aq, 50*4.6 mm, 1.8 μm, Mobile Phase A: Water/0.05% TFA, Mobile Phase B: Acetonitrile, 5%-95% B-5 min, 1.50 mL/min]: T_{R}=1.65 min; [M+H]⁺: 481. Chiral SFC [condition: Column Name: CHIRALPACK IF-3 50*3.0 mm,3.0 μm, Co-Solvent: MeOH (0.2% MSA), Gradient (B %): 10% to 50% in 2.0 min,hold 1.0 min at 50%, Flow (mL/min): 2.00 mL/min]: T_{R}=1.76 min (major peak), T_{R}=1.90 min (minor peak); dr=95.5:4.5.

### Synthesis of tert-butyl (azetidin-3-ylmethyl)(4-fluorophenethyl)carbamate (165a) Benzyl 3-(((tert-butoxycarbonyl)(4-fluorophenethyl) amino) methyl) azetidine-1-carboxylate (169a)

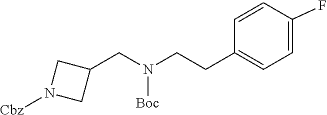 [SMILES: CC(C)(C)OC(=O)N(CCc1ccc(F)cc1)CC1CN(C(=O)OCc2ccccc2)C1]

A mixture of benzyl 3-(aminomethyl) azetidine-1-carboxylate (0.55 g, 2.50 mmol, 1.0 eq) and 2-(4-fluorophenyl)acetaldehyde (0.35 g, 2.50 mmol, 1.0 eq) were dissolved in DCM (5.0 mL) at room temperature. The resulting mixture was stirred for 10 min at room temperature then NaBH₄ (0.10 g, 2.70 mmol, 1.1 eq) was added. The mixture was stirred for 30 min at this temperature and then TEA (0.51 g, 5.00 mmol, 2.0 eq) and (Boc) 2O (1.09 g, 5.00 mmol, 2.0 eq) in MeOH (5.0 mL) was added. The reaction mixture was stirred for 0.5 h at room temperature, the resulting mixture was concentrated under reduced pressure and the crude residue was purified by silica gel column chromatography (40:60 AcOEt: petroleum ether) to afford benzyl-3-{[(tert-butoxycarbonyl) [2-(4-fluorophenyl)ethyl]amino]methyl}azetidine-1-carboxylate 169a (0.53 g, 48%) as a colorless oil. LCMS [Xbridge Shield C18, 50*3.0 mm, 3.5 μm; Mobile Phase A: Water/Ammonia, Mobile Phase B: CH₃CN, 5%-100% B-2.5 min, 1.50 mL/min, ES, m/z]: T_{R}=1.00 min; [M+H]⁺: 443.

### tert-Butyl(azetidin-3-ylmethyl)(4-fluorophenethyl)carbamate (165a)

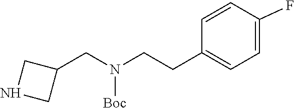 [SMILES: CC(C)(C)OC(=O)N(CCc1ccc(F)cc1)CC1CNC1]

To a solution of benzyl 3-{[(tert-butoxycarbonyl) [2-(4-fluorophenyl)ethyl]amino]methyl}azetidine-1-carboxylate 169a (0.53 g, 1.10 mmol, 1.0 eq) in MeOH (5.0 mL) was added 10% Pd/C(0.11 g) in a pressure tank. The reaction mixture was hydrogenated at room temperature under 30 psi of hydrogen pressure for 1 h. The resulting mixture was filtered through a Celite pad and concentrated under reduced pressure to afford the crude product tert-butyl-N-(azetidin-3-ylmethyl)-N-[2-(4-fluorophenyl)ethyl]carbamate 165a (0.33 g, 99%) was used in the next step directly without further purification. LCMS [Cortecs C_{18+, 50*3.0} mm, 2.7 μm; Mobile Phase A: Water/0.5% TFA, Mobile Phase B: CH₃CN, 2%-100% B-2 min (+), 1.50 mL/min, ES, m/z]: T_{R}=0.84 min; [M+H]⁺: 309.

### Synthesis of (1R,2S,3R,5S)-3-(4-amino-5-(thiazol-2-yl)-7H-pyrrolo[2,3-d]pyrimidin-7-yl)-5-(azetidin-3-ylmethyl) cyclopentane-1,2-diol (486)

### tert-Butyl 3-(((3αR,4R,6αR)-2,2-dimethyl-6-oxotetrahydro-4H-cyclopenta[d][1,3]dioxol-4-yl)methylene) azetidine-1-carboxylate (13b)

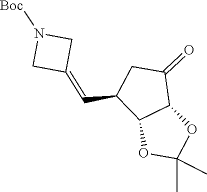 [SMILES: CC(C)(C)OC(=O)N1CC(=C[C@H]2CC(=O)[C@@H]3OC(C)(C)O[C@H]23)C1]

Into a 20 mL sealed tube were added (3αR,6αR)-2,2-dimethyl-3αH,6αH-cyclopenta[d][1,3]dioxol-4-one (800 mg, 5.18 mmol, 1.0 eq), tert-butyl 3-[(4,4,5,5-tetramethyl-1,3,2-dioxaborolan-2-yl)methylidene]azetidine-1-carboxylate (2.29 g, 7.78 mmol, 1.5 eq) [Eur. J. Org. Chem., 2019, 2019 (33), 5624-5635], [R^{h} (COD) C_{1]2} (511 mg, 1.04 mmol, 0.2 eq), KOH (87 mg, 1.56 mmol, 0.3 eq), H₂O (1.0 mL) and CH₃OH (10.0 mL) at room temperature. The reaction mixture was irradiated with microwave radiation for 2 h at 40° C. The reaction was quenched with water (30 mL) and then extracted with EtOAc (3×30 mL). The organic layer was dried over anhydrous Na₂SO₄, filtered and the filtrate was concentrated under reduced pressure. The residue was purified by silica gel column chromatography (95:5 AcOEt: Petroleum ether) to afford tert-butyl 3-{[(3αR,4R,6αR)-2,2-dimethyl-6-oxo-tetrahydrocyclopenta[d][1,3]dioxol-4-yl]methylidene}azetidine-1-carboxylate 13b (860 mg, 51%) as an off-white solid. LCMS [XBridge C18, 50*3.0 mm, 3.5 μm; Mobile Phase A: Water/5 mM NH₄HCO₃, Mobile Phase B: Methanol, 10%-95% B-3 min, 1.00 mL/min, ES, m/z]: T_{R}=1.98 min, ES m/z [M+H]⁺: 324.

### tert-Butyl 3-(((3αR,4S,6αR)-2,2-dimethyl-6-oxotetrahydro-4H-cyclopenta[d][1,3]dioxol-4-yl)methyl) azetidine-1-carboxylate (14b)

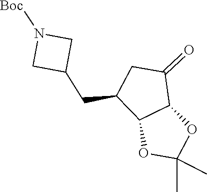 [SMILES: CC(C)(C)OC(=O)N1CC(C[C@H]2CC(=O)[C@@H]3OC(C)(C)O[C@H]23)C1]

To a solution of tert-butyl 3-{[(3αR,4R,6αR)-2,2-dimethyl-6-oxo-tetrahydrocyclopenta[d][1,3]dioxol-4-yl]methylidene}azetidine-1-carboxylate 13b (860 mg, 2.66 mmol, 1.0 eq) in MeOH (10 mL) was added 10% Pd/C(56 mg) in a 40 mL vial. The mixture was hydrogenated at room temperature for 1 h under a hydrogen balloon, filtered through a Celite pad and concentrated under reduced pressure to afford tert-butyl 3-{[(3αR,4S,6αR)-2,2-dimethyl-6-oxo-tetrahydrocyclopenta[d][1,3]dioxol-4-yl]methyl}azetidine-1-carboxylate 14b (740 mg, 85%) as an off-white crude solid. LCMS [XBridge C18, 50*3.0 mm, 3.5 μm; Mobile Phase A: Water/5 mM NH₄HCO₃, Mobile Phase B: Methanol, 10%-95% B-3 min, 1.00 mL/min, ES, m/z]: T_{R}=2.00 min, ES m/z [M+H]⁺: 326.

### tert-Butyl 3-(((3αR,4S,6αS)-6-hydroxy-2,2-dimethyltetrahydro-4H-cyclopenta[d][1,3]dioxol-4-yl)methyl) azetidine-1-carboxylate (15b)

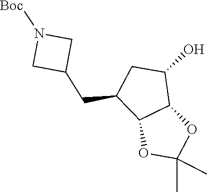 [SMILES: CC(C)(C)OC(=O)N1CC(C[C@H]2C[C@H](O)[C@@H]3OC(C)(C)O[C@H]23)C1]

To a stirred solution of tert-butyl 3-{[(3αR,4S,6αR)-2,2-dimethyl-6-oxo-tetrahydrocyclopenta[d][1,3]dioxol-4-yl]methyl}azetidine-1-carboxylate 14b (740 mg, 2.27 mmol, 1.0 eq) in MeOH (10.0 mL) were added NaBH₄ (129 mg, 3.41 mmol, 1.5 eq) in portions at 0° C. The resulting mixture was stirred for 1 h at room temperature. The reaction was quenched with water (30 mL) and then extracted with EtOAc (3×30 mL). The organic layer was dried over anhydrous Na₂SO₄, filtered and the filtrate was concentrated under reduced pressure. The crude product was purified by Prep-HPLC [Column: Xtimate C18 50×250 mm, 10 μm; Mobile Phase A: 0.5% NH₃.H₂O, Mobile Phase B: ACN; Flow rate: 90 mL/min; Gradient: 90% 15 min; Detector, UV 220 nm] to afford tert-butyl 3-{[(3αR,4S,6αS)-6-hydroxy-2,2-dimethyl-tetrahydro-3αH-cyclopenta[d][1,3]dioxol-4-yl]methyl}azetidine-1-carboxylate 15b (550 mg, 73%) as an off-white solid. LCMS [L-column3 ODS, 50*3.0 mm, 3.0 μm; Mobile Phase A: Water/5 mM NH₄HCO₃+0.05% Ammonia, Mobile Phase B: Acetonitrile/5% water, 5%-100% B-3 min, 1.00 mL/min, ES, m/z]: T_{R}=1.62 min, ES m/z [M+H]⁺: 328.

### tert-Butyl 3-(((3αR,4S,6αR)-2,2-dimethyl-6-(((trifluoromethyl) sulfonyl)oxy) tetrahydro-4H-cyclopenta[d][1,3]dioxol-4-yl)methyl) azetidine-1-carboxylate (16h)

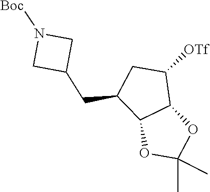 [SMILES: CC(C)(C)OC(=O)N1CC(C[C@H]2C[C@H](OS(=O)(=O)C(F)(F)F)[C@@H]3OC(C)(C)O[C@H]23)C1]

Into a 40 mL vial were added tert-butyl 3-{[(3αR,4S,6αS)-6-hydroxy-2,2-dimethyl-tetrahydro-3αH-cyclopenta[d][1,3]dioxol-4-yl]methyl}azetidine-1-carboxylate 15b (550 mg, 1.68 mmol, 1.0 eq), DCM (10.0 mL) and pyridine (332 mg, 4.20 mmol, 2.5 eq) at room temperature. Tf₂O (947 mg, 3.36 mmol, 2.0 eq) was added dropwise at 0° C. The resulting mixture was stirred for 1 h at room temperature, then concentrated under reduced pressure to afford tert-butyl 3-{[(3αR,4S,6αR)-2,2-dimethyl-6-(trifluoromethanesulfonyloxy)-tetrahydro-3αH-cyclopenta[d][1,3]dioxol-4-yl]methyl}azetidine-1-carboxylate 16b (500 mg, crude) as a yellow oil. The crude product was used in the next step directly without further purification. LCMS [L-column3 ODS, 50*3.0 mm, 3.0 μm; Mobile Phase A: Water/5 mM NH₄HCO₃+0.05% Ammonia, Mobile Phase B: Acetonitrile/5% water, 5%-100% B-3 min, 1.00 mL/min, ES, m/z]: T_{R}=2.21 min, ES m/z [M+H]⁺: 460.

### tert-Butyl 3-(((3αR,4S,6R,6αS)-6-(5-bromo-4-chloro-7H-pyrrolo[2,3-d]pyrimidin-7-yl)-2,2-dimethyltetrahydro-4H-cyclopenta[d][1,3]dioxol-4-yl) methyl) azetidine-1-carboxylate (17b)

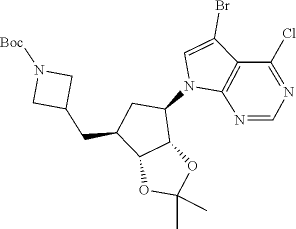 [SMILES: CC(C)(C)OC(=O)N1CC(C[C@H]2C[C@@H](n3cc(Br)c4c(Cl)ncnc43)[C@@H]3OC(C)(C)O[C@H]23)C1]

To a stirred solution of tert-butyl 3-{[(3αR,4S,6αR)-2,2-dimethyl-6-(trifluoromethanesulfonyloxy)-tetrahydro-3αH-cyclopenta[d][1,3]dioxol-4-yl]methyl}azetidine-1-carboxylate 16b (500 mg, 1.08 mmol, 1.0 eq) and 5-bromo-4-chloro-7H-pyrrolo[2,3-d]pyrimidine (303 mg, 1.30 mmol, 1.2 eq) in DMF (5.0 mL) was added 60% NaH (104 mg, 4.35 mmol, 4.0 eq) in portions at 0° C. The resulting mixture was stirred for 1 h at room temperature. The reaction was quenched with water (30 mL) and then extracted with EtOAc (3×30 mL). The organic layer was dried over anhydrous Na₂SO₄, filtered and the filtrate was concentrated under reduced pressure. The residue was purified by silica gel column chromatography (1:1 AcOEt: Petroleum ether) to afford tert-butyl 3-{[(3αR,4S,6R,6αS)-6-{5-bromo-4-chloropyrrolo[2,3-d]pyrimidin-7-yl}-2,2-dimethyl-tetrahydro-3αH-cyclopenta[d][1,3]dioxol-4-yl]methyl}azetidine-1-carboxylate 17b (350 mg, 59%) as a yellow solid. LCMS [XBridge C18, 50*3.0 mm, 3.5 μm; Mobile Phase A: Water/5 mM NH₄HCO₃, Mobile Phase B: Methanol, 10%-95% B-3 min, 1.00 mL/min, ES, m/z]: T_{R}=2.39 min, ES m/z [M+H]⁺: 541, 543.

### tert-Butyl 3-(((3αR,4S,6R,6αS)-6-(4-amino-5-bromo-7H-pyrrolo[2,3-d]pyrimidin-7-yl)-2,2-dimethyltetrahydro-4H-cyclopenta[d][1,3]dioxol-4-yl)methyl) azetidine-1-carboxylate (18b)

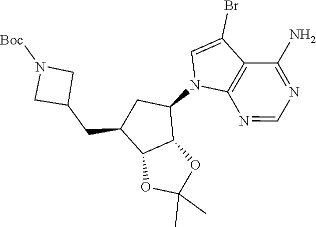 [SMILES: CC(C)(C)OC(=O)N1CC(C[C@H]2C[C@@H](n3cc(Br)c4c(N)ncnc43)[C@@H]3OC(C)(C)O[C@H]23)C1]

Into a 40 mL vial were added tert-butyl 3-{[(3αR,4S,6R,6αS)-6-{5-bromo-4-chloropyrrolo[2,3-d]pyrimidin-7-yl}-2,2-dimethyl-tetrahydro-3αH-cyclopenta[d][1,3]dioxol-4-yl]methyl}azetidine-1-carboxylate 17b (350 mg, 0.64 mmol, 1.0 eq) and 7M NH₃ in MeOH (5.0 mL) at room temperature. The resulting mixture was stirred for 16 h at 70° C. The resulting mixture was concentrated under reduced pressure. The residue was purified by silica gel column chromatography (1:1 AcOEt: petroleum ether) to afford tert-butyl 3-{[(3αR,4S,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}azetidine-1-carboxylate 18b (240 mg, 71%) as a yellow solid. LCMS [XBridge C18, 50*3.0 mm, 3.5 μm; Mobile Phase A: Water/5 mM NH₄HCO₃, Mobile Phase B: Methanol, 10%-95% B-3 min, 1.00 mL/min, ES, m/z]: T_{R}=2.20 min, ES m/z [M+H]⁺: 522, 524.

### tert-Butyl 3-(((3αR,4S,6R,6αS)-6-(4-amino-5-(thiazol-2-yl)-7H-pyrrolo[2,3-d]pyrimidin-7-yl)-2,2-dimethyltetrahydro-4H-cyclopenta[d][1,3]dioxol-4-yl)methyl) azetidine-1-carboxylate (19b)

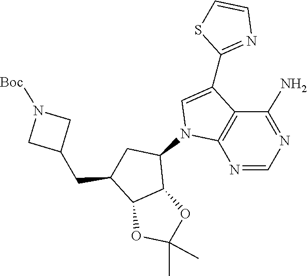 [SMILES: CC(C)(C)OC(=O)N1CC(C[C@H]2C[C@@H](n3cc(-c4nccs4)c4c(N)ncnc43)[C@@H]3OC(C)(C)O[C@H]23)C1]

To a solution of tert-butyl 3-{[(3αR,4S,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}azetidine-1-carboxylate 18b (100 mg, 0.19 mmol, 1.0 eq) and 2-(tributylstannyl)-1,3-thiazole (107 mg, 0.28 mmol, 1.5 eq) in toluene (2.0 mL) were added Pd (PPh₃) 2Cl₂ (26 mg, 0.04 mmol, 0.2 eq). After stirring for 2 h at 120° C. under a nitrogen atmosphere, the resulting mixture was concentrated under reduced pressure, then extracted with EtOAc (3×10 mL). The organic layer was dried over anhydrous Na₂SO₄, filtered and the filtrate was concentrated under reduced pressure. The residue was purified by silica gel column chromatography (1:1 AcOEt: petroleum ether) to afford tert-butyl 3-{[(3αR,4S,6R,6αS)-6-[4-amino-5-(1,3-thiazol-2-yl) pyrrolo[2,3-d]pyrimidin-7-yl]-2,2-dimethyl-tetrahydro-3αH-cyclopenta[d][1,3]dioxol-4-yl]methyl}azetidine-1-carboxylate 19b (65 mg, 64%) as a yellow solid. LCMS [L-column3 ODS, 50*3.0 mm, 3.0 μm; Mobile Phase A: Water/5 mM NH₄HCO₃+0.05% Ammonia, Mobile Phase B: Acetonitrile/5% water, 5%-100% B-3 min, 1.50 mL/min, ES, m/z]: T_{R}=2.01 min, ES m/z [M+H]⁺: 527.

### (1R,2S,3R,5S)-3-(4-amino-5-(thiazol-2-yl)-7H-pyrrolo[2,3-d]pyrimidin-7-yl)-5-(azetidin-3-ylmethyl) cyclopentane-1,2-diol (486)

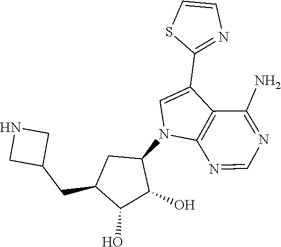 [SMILES: Nc1ncnc2c1c(-c1nccs1)cn2[C@@H]1C[C@H](CC2CNC2)[C@@H](O)[C@H]1O]

Into a 8 mL vial were added tert-butyl 3-{[(3αR,4S,6R,6αS)-6-[4-amino-5-(1,3-thiazol-2-yl) pyrrolo[2,3-d]pyrimidin-7-yl]-2,2-dimethyl-tetrahydro-3αH-cyclopenta[d][1,3]dioxol-4-yl]methyl}azetidine-1-carboxylate 19b (65 mg, 0.12 mmol, 1.0 eq) and TFA (2.0 mL) at room temperature. The resulting mixture was stirred for 1 h at 50° C., then concentrated under reduced pressure and basified to pH>8 with NH₃.H₂O. The mixture was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 ml/min; Gradient: 20% B to 55% B in 9 min; Detector, UV 220&254 nm] to afford (1R,2S,3R,5S)-3-[4-amino-5-(1,3-thiazol-2-yl) pyrrolo[2,3-d]pyrimidin-7-yl]-5-(azetidin-3-ylmethyl) cyclopentane-1,2-diol 486 (13.7 mg, 28%) as an off-white solid. ¹H NMR (300 MHZ, DMSO-d₆+D₂O) 8 8.14 (s, 1H), 8.09 (s, 1H), 7.81 (d, J=3.3 Hz, 1H), 7.60 (d, J=3.3 Hz, 1H), 4.90-4.76 (m, 1H), 4.25 (dd, J=8.0, 6.2 Hz, 1H), 3.82-3.76 (m, 2H), 3.73-3.63 (m, 1H), 3.44-3.38 (m, 2H), 2.93-2.46 (m, 1H), 2.19-2.05 (m, 1H), 1.83-1.77 (m, 2H), 1.70-1.58 (m, 1H), 1.57-1.41 (m, 1H). LCMS [ZORBAX SB-Aq, 50*4.6 mm, 1.8 μm; Mobile Phase A: Water/0.02% TFA, Mobile Phase B: Acetonitrile, 5%-95% B-3 min, 1.50 mL/min, ES, m/z]: T_{R}=0.96 min, ES m/z [M+H]⁺: 387. Chiral-SFC [Cellulose SB 50*3.0 mm,3.0 μm, Solvent B: MeOH (0.2% MSA), Oven Temperature: 35° C., Total Flow: 2.00 mL/min. Gradient 30% to 50% in 2.0 min, hold 1.0 min at 50%]: T_{R}=1.73 min (3 min run), single peak.

### Synthesis of (1R,2S,3R,5R)-3-(4-amino-5-(thiazol-2-yl)-7H-pyrrolo[2,3-d]pyrimidin-7-yl)-5-(piperidin-4-yl) cyclopentane-1,2-diol (487)

### tert-Butyl 4-((3αR,4R,6αR)-2,2-dimethyl-6-oxotetrahydro-4H-cyclopenta[d][1,3]dioxol-4-yl)-3,6-dihydropyridine-1 (2H)-carboxylate (166a)

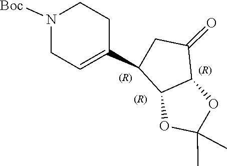

To (3αR,6αR)-2,2-dimethyl-3αH,6αH-cyclopenta[d][1,3]dioxol-4-one (0.80 g, 5.19 mmol, 1.0 eq) in a sealed tube was added tert-butyl 4-(4,4,5,5-tetramethyl-1,3,2-dioxaborolan-2-yl)-3,6-dihydro-2H-pyridine-1-carboxylate (2.40 g, 7.78 mmol, 1.5 eq), [R^{h} (COD) C_{1]2} (0.26 g, 0.52 mmol, 0.1 eq), KOH (58 mg, 1.04 mmol, 0.2 eq), H₂O (0.34 mL) and MeOH (36.0 mL) at room temperature and the reaction mixture was stirred under microwave radiation at 40° C. for 2 h. The resulting mixture was concentrated under reduced pressure and the residue was purified by silica gel column chromatography (5:95 AcOEt: petroleum ether) to afford tert-butyl 4-((3αR,4R,6αR)-2,2-dimethyl-6-oxotetrahydro-4H-cyclopenta[d][1,3]dioxol-4-yl)-3,6-dihydropyridine-1 (2H)-carboxylate 166a (0.80 g, 46%) as a yellow oil. ¹H NMR (400 MHZ, Chloroform-d) 85.34 (s, 1H), 4.67-4.63 (m, 1H), 4.28-4.21 (m, 1H), 3.89-3.84 (m, 2H), 3.54-3.47 (m, 2H), 2.97-2.80 (m, 2H), 2.36-2.28 (m, 1H), 2.25-2.16 (m, 1H), 2.12-2.05 (m, 1H), 1.47 (s, 9H), 1.46 (s, 3H), 1.36 (s, 3H). LCMS [XBridge C18, 50*3.0 mm, 3.5 μm; Mobile Phase A: Water/5 mM NH₄HCO₃, Mobile Phase B: Methanol, 10%-95% B-3 min (+), 1.00 mL/min, ES, m/z]: T_{R}=2.06 min; [M+H]⁺: 338.

### tert-Butyl 4-((3αR,4R,6αR)-2,2-dimethyl-6-oxotetrahydro-4H-cyclopenta[d][1,3]dioxol-4-yl) piperidine-1-carboxylate (167a)

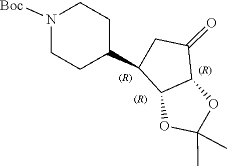

To a solution of tert-butyl 4-[(3αR,4R,6αR)-2,2-dimethyl-6-oxo-tetrahydrocyclopenta[d][1,3]dioxol-4-yl]-3,6-dihydro-2H-pyridine-1-carboxylate 167a (0.80 g, 2.37 mmol, 1.0 eq) in 10.0 mL MeOH was added 10% Pd/C(75 mg, 0.71 mmol, 0.3 eq) in a pressure tank. The mixture was hydrogenated at room temperature under 10 psi of hydrogen pressure for 1 h, filtered through a Celite pad and concentrated under reduced pressure to afford tert-butyl 4-((3αR,4R,6αR)-2,2-dimethyl-6-oxotetrahydro-4H-cyclopenta[d][1,3]dioxol-4-yl) piperidine-1-carboxylate 192a (0.70 g, crude) as a yellow oil. LCMS [XBridge C18, 50*3.0 mm, 3.5 μm; Mobile Phase A: Water/5 mM NH₄HCO₃, Mobile Phase B: Methanol, 10%-95% B-3 min (+), 1.00 mL/min, ES, m/z]: T_{R}=2.04 min; [M+H]⁺: 340.

### tert-Butyl 4-((3αR,4R,6S,6αS)-6-hydroxy-2,2-dimethyltetrahydro-4H-cyclopenta[d][1,3]dioxol-4-yl) piperidine-1-carboxylate (168a)

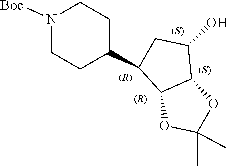

To a solution of tert-butyl 4-[(3αR,4R,6αR)-2,2-dimethyl-6-oxo-tetrahydrocyclopenta[d][1,3]dioxol-4-yl]piperidine-1-carboxylate 167a (0.70 g, 2.06 mmol, 1.0 eq) in MeOH (10.0 mL) was added NaBH₄ (117 mg, 3.09 mmol, 1.5 eq) at 0° C. and the reaction mixture was stirred at 0° C. for 1 h. The resulting mixture was quenched by the addition of water (1.0 mL) at 0° C., filtered and the filter cake was washed with MeOH (3×2.0 mL). The combined filtrate was concentrated under reduced pressure and the crude product was purified by Prep-HPLC [Column: Xtimate C18 50×250 mm, 10 μm; Mobile Phase A: 0.5% NH₃.H₂O, Mobile Phase B: ACN; Flow rate: 90 mL/min; Gradient: 90% 15 min; Detector, UV 220 nm]: to afford tert-butyl 4-((3αR,4R,6S,6αS)-6-hydroxy-2,2-dimethyltetrahydro-4H-cyclopenta[d][1,3]dioxol-4-yl) piperidine-1-carboxylate 168a (0.50 g, 71%) as a yellow oil. ¹H NMR (400 MHZ, Chloroform-d) 84.51-4.46 (m, 1H), 4.39-4.35 (m, 1H), 4.16-4.08 (m, 2H), 4.07-4.02 (m, 1H), 2.75-2.61 (m, 2H), 2.52 (s, 1H), 2.09-1.95 (m, 2H), 1.85-1.77 (m, 1H), 1.67-1.58 (m, 1H), 1.58-1.53 (m, 1H), 1.53 (s, 3H), 1.46 (s, 9H), 1.37 (s, 3H), 1.32-1.08 (m, 3H). LCMS [XBridge C18, 50*3.0 mm, 3.5 μm; Mobile Phase A: Water/5 mM NH₄HCO₃, Mobile Phase B: Methanol, 10%-95% B-3 min (+), 1.00 mL/min, ES, m/z]: T_{R}=2.03 min; [M+H]⁺: 342.

### tert-Butyl 4-((3αR,4R,6S,6αR)-2,2-dimethyl-6-(((trifluoromethyl) sulfonyl)oxy) tetrahydro-4H-cyclopenta[d][1,3]dioxol-4-yl) piperidine-1-carboxylate (169a)

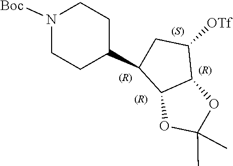

To a solution of tert-butyl 4-[(3αR,4R,65,6αS)-6-hydroxy-2,2-dimethyl-tetrahydro-3αH-cyclopenta[d][1,3]dioxol-4-yl]piperidine-1-carboxylate 168a (500 mg, 1.46 mmol, 1.0 eq) in DCM (5.0 mL) was added pyridine (347 mg, 4.40 mmol, 3.0 eq) and Tf₂O (826 mg, 2.93 mmol, 2.0 eq) at room temperature and the reaction mixture was stirred for 1 h at room temperature. The resulting mixture was concentrated under reduced pressure to afford tert-butyl 4-[(3αR,4R,6S,6αR)-2,2-dimethyl-6-(trifluoromethanesulfonyloxy)-tetrahydro-3αH-cyclopenta[d][1,3]dioxol-4-yl]piperidine-1-carboxylate 169a (600 mg, crude) as a yellow oil. The crude product was used in the next step without purification. LCMS [L-column3 ODS, 50*3.0 mm, 3.0 μm; Mobile Phase A: Water/5 mM NH₄HCO₃+0.05% Ammonia, Mobile Phase B: Acetonitrile/5% water, 5%-100% B-3 min (+), 1.00 mL/min, ES, m/z]: T_{R}=2.24 min; [M+H]⁺: 474.

### tert-Butyl 4-((3αR,4R,6R,6αS)-6-(5-bromo-4-chloro-7H-pyrrolo[2,3-d]pyrimidin-7-yl)-2,2-dimethyltetrahydro-4H-cyclopenta[d][1,3]dioxol-4-yl) piperidine-1-carboxylate (170a)

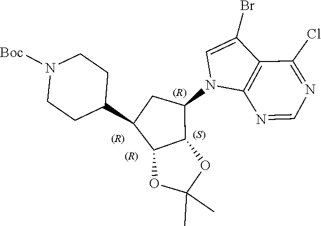

To a solution of tert-butyl 4-[(3αR,4R,6S,6αR)-2,2-dimethyl-6-(trifluoromethanesulfonyloxy)-tetrahydro-3αH-cyclopenta[d][1,3]dioxol-4-yl]piperidine-1-carboxylate 169a (600 mg, 1.27 mmol, 1.0 eq) and 5-bromo-4-chloro-7H-pyrrolo[2,3-d]pyrimidine (353 mg, 1.52 mmol, 1.2 eq) in DMF (15 mL), at 0° C., was added NaH (62 mg, 2.53 mmol, 2.0 eq), in several portions over 5 min, and the reaction mixture was stirred for 1 h at room temperature. The reaction was quenched with H₂O (10 mL) at 0° C. and the aqueous layer was extracted with AcOEt (3×10 mL). The combined organic layers were dried over anhydrous Na₂SO₄, filtered and then the filtrate was concentrated under reduced pressure. The crude residue was purified by silica gel column chromatography (AcOEt: petroleum ether 20:80) to afford tert-butyl 4-[(3αR,4R,6R,6αS)-6-{5-bromo-4-chloropyrrolo[2,3-d]pyrimidin-7-yl}-2,2-dimethyl-tetrahydro-3αH-cyclopenta[d][1,3]dioxol-4-yl]piperidine-1-carboxylate 170a (220 mg, 31%) as a yellow solid. ¹H NMR (400 MHZ, Chloroform-d) 88.63 (s, 1H), 7.36 (s, 1H), 4.98-4.87 (m, 2H), 4.58-4.51 (m, 1H), 4.23-4.07 (m, 2H), 2.74-2.65 (m, 2H), 2.51-2.39 (m, 1H), 2.29-2.09 (m, 2H), 2.02-1.88 (m, 2H), 1.70-1.60 (m, 2H), 1.56 (s, 3H), 1.47 (s, 9H), 1.30 (s, 3H), 1.25-1.15 (m, 1H). LCMS [L-column3 ODS, 50*3.0 mm, 3.0 μm; Mobile Phase A: Water/5 mM NH₄HCO₃+0.05% Ammonia, Mobile Phase B: Acetonitrile/5% water, 5%-100% B-3 min (+), 1.00 mL/min, ES, m/z]: T_{R}=2.40 min; [M+H]⁺: 555, 557.

### tert-Butyl 4-((3αR,4R,6R,6αS)-6-(4-amino-5-bromo-7H-pyrrolo[2,3-d]pyrimidin-7-yl)-2,2-dimethyltetrahydro-4H-cyclopenta[d][1,3]dioxol-4-yl) piperidine-1-carboxylate (171a)

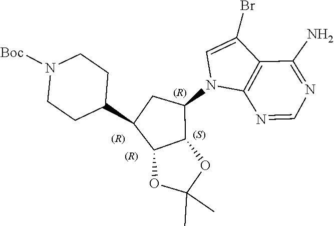

To tert-butyl 4-[(3αR,4R,6R,6αS)-6-{5-bromo-4-chloropyrrolo[2,3-d]pyrimidin-7-yl}-2,2-dimethyl-tetrahydro-3αH-cyclopenta[d][1,3]dioxol-4-yl]piperidine-1-carboxylate 170a (105 mg, 0.19 mmol, 1.0 eq) was added NH₃ (7M in MeOH, 3.0 mL) at room temperature and the reaction mixture was stirred at 75° C. for 16 h. The resulting mixture was concentrated under reduced pressure and the crude residue was purified by silica gel column chromatography (AcOEt: petroleum ether 20:80) to afford tert-butyl 4-[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]piperidine-1-carboxylate 171a (80 mg, 79%) as a yellow solid. ¹H NMR (400 MHZ, Chloroform-d) 88.26 (s, 1H), 7.03 (s, 1H), 5.76 (br s, 2H), 4.99-4.78 (m, 2H), 4.59-4.49 (m, 1H), 4.23-4.03 (m, 2H), 2.81-2.65 (m, 2H), 2.48-2.37 (m, 1H), 2.19-1.83 (m, 5H), 1.70-1.60 (m, 1H), 1.56 (s, 3H), 1.48 (s, 9H), 1.31 (s, 3H), 1.27-1.16 (m, 1H). LCMS [XBridge C18, 50*3.0 mm, 3.5 μm; Mobile Phase A: Water/5 mM NH₄HCO₃, Mobile Phase B: Methanol, 10%-95% B-3 min (+), 1.00 mL/min, ES, m/z]: T_{R}=2.24 min; [M+H]⁺: 536, 538.

### tert-Butyl 4-((3αR,4R,6R,6αS)-6-(4-amino-5-(thiazol-2-yl)-7H-pyrrolo[2,3-d]pyrimidin-7-yl)-2,2-dimethyltetrahydro-4H-cyclopenta[d][1,3]dioxol-4-yl) piperidine-1-carboxylate (172a)

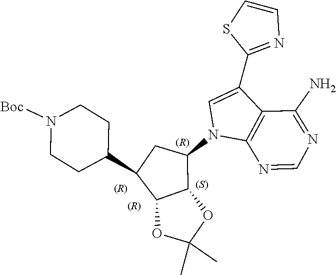

To a solution of tert-butyl 4-[(3αR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]piperidine-1-carboxylate 171a (80 mg, 0.15 mmol, 1.0 eq) in toluene (1.0 mL) was added Pd (PPh₃) 2Cl₂ (31 mg, 0.05 mmol, 0.3 eq), and 2-(tributylstannyl)-1,3-thiazole (167 mg, 0.45 mmol, 3.0 eq), in a sealed tube, at room temperature and the reaction mixture was stirred at 120° C. for 16 h, under nitrogen. The resulting mixture was concentrated under reduced pressure and the crude residue was purified by Prep-TLC(50:50 THF: petroleum ether) to afford tert-butyl 4-[(3αR,4R,6R,6αS)-6-[4-amino-5-(1,3-thiazol-2-yl) pyrrolo[2,3-d]pyrimidin-7-yl]-2,2-dimethyl-tetrahydro-3αH-cyclopenta[d][1,3]dioxol-4-yl]piperidine-1-carboxylate 172a (50 mg, 62%) as a yellow solid. ¹H NMR (400 MHZ, Chloroform-d) 89.61 (br, s, 1H), 8.26 (s, 1H), 7.70 (d, J=3.4 Hz, 1H), 7.49 (s, 1H), 7.18 (d, J=3.4 Hz, 1H), 5.77 (br, s, 1H), 5.11-5.01 (m, 1H), 4.94-4.80 (m, 1H), 4.58 (dd, J=7.6, 6.2 Hz, 1H), 4.25-4.04 (m, 2H), 2.82-2.62 (m, 2H), 2.50-2.38 (m, 1H), 2.30-2.13 (m, 1H), 1.98-1.87 (m, 2H), 1.71-1.62 (m, 1H), 1.57 (s, 3H), 1.51 (s, 1H), 1.47 (s, 9H), 1.32 (s, 3H), 1.30-1.10 (m, 2H). LCMS [XBridge C18, 50*3.0 mm, 3.5 μm; Mobile Phase A: Water/5 mM NH₄HCO₃, Mobile Phase B: Methanol, 10%-95% B-5 min (+), 1.00 mL/min, ES, m/z]: T_{R}=3.70 min; [M+H]⁺: 541.

### (1R,2S,3R,5R)-3-(4-Amino-5-(thiazol-2-yl)-7H-pyrrolo[2,3-d]pyrimidin-7-yl)-5-(piperidin-4-yl) cyclopentane-1,2-diol (487)

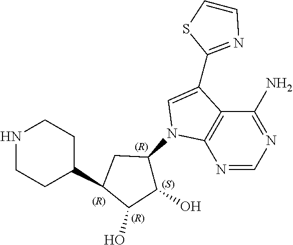

To tert-butyl 4-[(3αR,4R,6R,6αS)-6-[4-amino-5-(1,3-thiazol-2-yl) pyrrolo[2,3-d]pyrimidin-7-yl]-2,2-dimethyl-tetrahydro-3αH-cyclopenta[d][1,3]dioxol-4-yl]piperidine-1-carboxylate 172a (50 mg, 0.09 mmol, 1.0 eq) was added TFA (1.0 mL) at room temperature and the reaction mixture was stirred at 50° C. for 1 h. The resulting mixture was concentrated under reduced pressure and residue was dissolved in DMF (2.0 mL) and basified to pH=10 with NH₃.H₂O. The crude mixture was purified by Prep-HPLC [Column: YMC-Actus Triart C18 ExRS, 30*150 mm, 5 μm; Mobile Phase A: Water (10 mmol/L NH₄HCO₃+0.1% NH₃.H₂O), Mobile Phase B: ACN; Flow rate: 30 mL/min; Gradient: 20% B to 55% B in 8 min, 55% B; Detector, UV 220&254 nm]: to afford (1R,2S,3R,5R)-3-[4-amino-5-(1,3-thiazol-2-yl) pyrrolo[2,3-d]pyrimidin-7-yl]-5-(piperidin-4-yl) cyclopentane-1,2-diol 487 (18.9 mg, 51%) as an off-white solid. ¹H NMR (400 MHZ, DMSO-d+D₂O) 88.15 (s, 1H), 8.09 (s, 1H), 7.80 (d, J=3.4 Hz, 1H), 7.59 (d, J=3.4 Hz, 1H), 4.90-4.76 (m, 1H), 4.28-4.17 (m, 1H), 3.85-3.71 (m, 1H), 3.03-2.86 (m, 2H), 2.48-2.36 (m, 2H), 2.11-1.97 (m, 1H), 1.85-1.76 (m, 1H), 1.70-1.48 (m, 3H), 1.46-1.31 (m, 1H), 1.21-1.00 (m, 2H). LCMS [Cortecs C18+, 50*3.0 mm, 2.7 μm; Mobile Phase A: Water/0.02% TFA, Mobile Phase B: Acetonitrile, 5%-95% B-3 min, 1.50 mL/min, ES, m/z]: T_{R}=0.53 min; [M+H]⁺: 401. Chiral-SFC [Cellulose SB 50*3.0 mm,3.0 μm, Solvent B: MeOH (0.2% MSA), Oven Temperature: 35° C., Total Flow: 2.00 mL/min. Gradient 10% to 50% in 2.0 min, hold 1.0 min at 50%]: T_{R}=1.72 min, single peak.

### Synthesis of (1R,2S,3R,5R)-3-(4-amino-5-(4-benzylthiazol-2-yl)-7H-pyrrolo[2,3-d]pyrimidin-7-yl)-5-(piperidin-4-yl) cyclopentane-1,2-diol (488)

### tert-Butyl 4-((3αR,4R,6R,6αS)-6-(4-chloro-5-iodo-7H-pyrrolo[2,3-d]pyrimidin-7-yl)-2,2-dimethyltetrahydro-4H-cyclopenta[d][1,3]dioxol-4-yl) piperidine-1-carboxylate (27b)

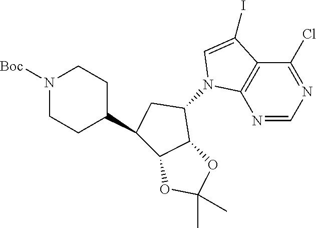 [SMILES: CC(C)(C)OC(=O)N1CCC([C@H]2C[C@H](n3cc(I)c4c(Cl)ncnc43)[C@@H]3OC(C)(C)O[C@@H]32)CC1]

To a stirred solution of tert-butyl 4-[(3αR,4R,6S,6αR)-2,2-dimethyl-6-(trifluoromethanesulfonyloxy)-tetrahydro-3αH-cyclopenta[d][1,3]dioxol-4-yl]piperidine-1-carboxylate (200 mg, 0.42 mmol, 1.0 eq) and 4-chloro-5-iodo-7H-pyrrolo[2,3-d]pyrimidine (141 mg, 0.50 mmol, 1.2 eq) in DMF (4.0 mL) was added NaH (40 mg, 1.69 mmol, 4.0 eq) in portions at 0° C. The resulting mixture was stirred for 1 h at room temperature. The reaction was quenched with water (10 mL) and then extracted with EtOAc (3×10 mL). The organic layer was dried over anhydrous Na₂SO₄, filtered and the filtrate was concentrated under reduced pressure. The residue was purified by silica gel column chromatography (1:1 AcOEt: petroleum ether) to afford tert-butyl 4-[(3aR,4R,6R,6aS)-6-{4-chloro-5-iodopyrrolo[2,3-d]pyrimidin-7-yl}-2,2-dimethyl-tetrahydro-3aH-cyclopenta[d][1,3]dioxol-4-yl]piperidine-1-carboxylate 27 (78 mg, 30%) as a yellow solid. LCMS [XBridge C18, 50*3.0 mm, 3.5 um; Mobile Phase A: Water/5 mM NH4HCO₃, Mobile Phase B: Methanol, 10%-95% B-3 min, 1.00 mL/min, ES, m/z]: TR=2.40 min, ES m/z [M+H]⁺: 603, 605.

### tert-Butyl 4-((3aR,4R,6R,6aS)-6-(4-amino-5-iodo-7H-pyrrolo[2,3-d]pyrimidin-7-yl)-2,2-dimethyltetrahydro-4H-cyclopenta[d][1,3]dioxol-4-yl)piperidine-1-carboxylate (28b)

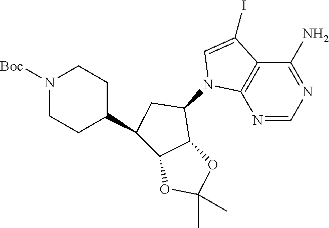 [SMILES: CC(C)(C)OC(=O)N1CCC([C@H]2C[C@@H](n3cc(I)c4c(N)ncnc43)[C@@H]3OC(C)(C)O[C@@H]32)CC1]

Into a 40 mL vial were added tert-butyl 4-[(3aR,4R,6R,6aS)-6-{4-chloro-5-iodopyrrolo[2,3-d]pyrimidin-7-yl}-2,2-dimethyl-tetrahydro-3aH-cyclopenta[d][1,3]dioxol-4-yl]piperidine-1-carboxylate 27b (75 mg, 0.12 mmol, 1.0 eq) and 7M NH3 in MeOH (3.0 mL) at room temperature. The resulting mixture was stirred for 16 h at 70° C. then concentrated under reduced pressure. The residue was purified by silica gel column chromatography (1:1 AcOEt: petroleum ether) to afford tert-butyl 4-[(3aR,4R,6R,6aS)-6-{4-amino-5-iodopyrrolo[2,3-d]pyrimidin-7-yl}-2,2-dimethyl-tetrahydro-3aH-cyclopenta[d][1,3]dioxol-4-yl]piperidine-1-carboxylate 28b (52 mg, 71%) as a yellow solid. LCMS [L-column3 ODS, 50*3.0 mm, 3.0 um; Mobile Phase A: Water/5 mM NH4HCO₃+0.05% Ammonia, Mobile Phase B: Acetonitrile/5% water, 5%-100% B-3 min, 1.20 mL/min, ES, m/z]: TR=2.11 min, ES m/z [M+H]⁺: 584.

### tert-Butyl 4-((3aR,4R,6R,6aS)-6-(4-amino-5-(4-benzylthiazol-2-yl)-7H-pyrrolo[2,3-d]pyrimidin-7-yl)-2,2-dimethyltetrahydro-4H-cyclopenta[d][1,3]dioxol-4-yl)piperidine-1-carboxylate (29b)

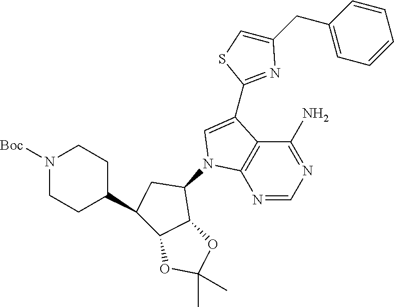 [SMILES: CC(C)(C)OC(=O)N1CCC([C@H]2C[C@@H](n3cc(-c4nc(Cc5ccccc5)cs4)c4c(N)ncnc43)[C@@H]3OC(C)(C)O[C@@H]32)CC1]

To a solution of tert-butyl 4-[(3aR,4R,6R,6aS)-6-{4-amino-5-iodopyrrolo[2,3-d]pyrimidin-7-yl}-2,2-dimethyl-tetrahydro-3aH-cyclopenta[d][1,3]dioxol-4-yl]piperidine-1-carboxylate 28b (52 mg, 0.09 mmol, 1.0 eq) and 4-benzyl-2-(tributylstannyl)-1,3-thiazole (62 mg, 0.13 mmol, 1.5 eq) [WO201110559]]in toluene (2.0 mL) were added Pd (PPh₃)₂Cl₂ (12 mg, 0.02 mmol, 0.2 eq). After stirring for 2 h at 120° C. under a nitrogen atmosphere, the resulting mixture was concentrated under reduced pressure, then extracted with EtOAc (3×10 mL). The organic layer was dried over anhydrous Na₂SO₄, filtered and the filtrate was concentrated under reduced pressure. The residue was purified by silica gel column chromatography (1:1 AcOEt: petroleum ether) to afford tert-butyl 4-[(3aR,4R,6R,6aS)-6-[4-amino-5-(4-benzyl-1,3-thiazol-2-yl)pyrrolo[2,3-d]pyrimidin-7-yl]-2,2-dimethyl-tetrahydro-3aH-cyclopenta[d][1,3]dioxol-4-yl]piperidine-1-carboxylate 29b (20 mg, 35%) as a yellow solid. LCMS [Halo C18, 30*3.0 mm, 2.0 um; Mobile Phase A: Water/0.05% TFA, Mobile Phase B: Acetonitrile, 5%-100% B-2 min, 1.50 mL/min, ES, m/z]: TR=1.22 min, ES m/z [M+H]⁺: 631.

### (1R,2S,3R,5R)-3-(4-amino-5-(4-benzylthiazol-2-yl)-7H-pyrrolo[2,3-d]pyrimidin-7-yl)-5-(piperidin-4-yl)cyclopentane-1,2-diol (488)

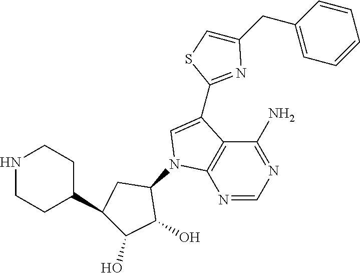 [SMILES: Nc1ncnc2c1c(-c1nc(Cc3ccccc3)cs1)cn2[C@@H]1C[C@H](C2CCNCC2)[C@@H](O)[C@H]1O]

Into a 8 mL vial were added tert-butyl 4-[(3aR,4R,6R,6aS)-6-[4-amino-5-(4-benzyl-1,3-thiazol-2-yl)pyrrolo[2,3-d]pyrimidin-7-yl]-2,2-dimethyl-tetrahydro-3aH-cyclopenta[d][1,3]dioxol-4-yl]piperidine-1-carboxylate 29b (20 mg, 0.03 mmol, 1.0 eq) and TFA (1.0 mL) at room temperature. The resulting mixture was stirred for 1 h at 50° C., then concentrated under reduced pressure. The mixture was basified to Ph>8 with NH3·H₂O. The crude product was purified by Prep-HPLC [Column: YMC-Actus Triart C18 ExRS, 30*150 mm, 5 um; Mobile Phase A: Water (10 mmol/L NH4HCO₃+0.1% NH3·H₂O), Mobile Phase B: ACN; Flow rate: 30 ml/min; Gradient: 20% B to 50% B in 8 min; Detector, UV 220&254 nm]to afford (1R,2S,3R,5R)-3-[4-amino-5-(4-benzyl-1,3-thiazol-2-yl)pyrrolo[2,3-d]pyrimidin-7-yl]-5-(piperidin-4-yl)cyclopentane-1,2-diol 488 (1.0 mg, 6%) as an off-white solid. ¹H NMR (300 MHZ, Acetonitrile-d₃) δ 8.08 (s, 1H), 7.80 (s, 1H), 7.40-7.18 (m, 5H), 7.04 (s, 1H), 4.87-4.77 (m, 1H), 4.25-4.20 (m, 1H), 4.19 (s, 2H), 3.98-3.88 (m, 1H), 3.44-3.20 (m, 2H), 2.92-2.75 (m, 2H), 2.31-2.14 (m, 1H), 2.14-2.04 (m, 1H), 1.88-1.78 (m, 2H), 1.71-1.53 (m, 2H), 1.45-1.39 (m, 2H), 1.30-1.24 (m, 1H).LCMS [ZORBAX SB-Aq, 50*4.6 mm, 1.8 um; Mobile Phase A: Water/0.02% TFA, Mobile Phase B: Acetonitrile, 5%-95% B-3 min, 1.50 mL/min, ES, m/z]: TR=1.22 min, ES m/z [M+H]⁺: 491.

### Synthesis of (1R, 2S,3R,5R)-3-(4-amino-5-(1-benzyl-1H-pyrazol-3-yl)-7H-pyrrolo[2,3-d]pyrimidin-7-yl)-5-(piperidin-4-yl)cyclopentane-1,2-diol (489)

### tert-Butyl 4-((3aR,4R,6R,6aS)-6-(4-amino-5-(1-benzyl-1H-pyrazol-3-yl)-7H-pyrrolo[2,3-d]pyrimidin-7-yl)-2,2-dimethyltetrahydro-4H-cyclopenta[d][1,3]dioxol-4-yl)piperidine-1-carboxylate (30b)

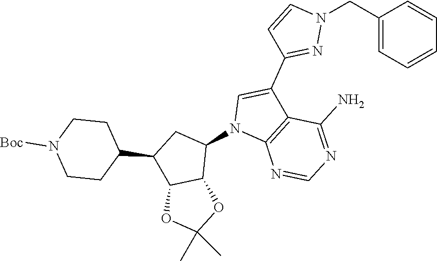 [SMILES: CC(C)(C)OC(=O)N1CCC([C@H]2C[C@@H](n3cc(-c4ccn(Cc5ccccc5)n4)c4c(N)ncnc43)[C@@H]3OC(C)(C)O[C@@H]32)CC1]

To a solution of tert-butyl 4-[(3aR,4R,6R,6aS)-6-{4-amino-5-bromopyrrolo[2,3-d]pyrimidin-7-yl}-2,2-dimethyl-tetrahydro-3aH-cyclopenta[d][1,3]dioxol-4-yl]piperidine-1-carboxylate 6 (50 mg, 0.09 mmol, 1.0 eq) and 1-benzyl-3-(4,4,5,5-tetramethyl-1,3,2-dioxaborolan-2-yl)pyrazole (31 mg, 0.11 mmol, 1.2 eq) [WO2017040450]in dioxane (2.0 mL) and H₂O (0.2 mL) were added K₃PO₄ (59 mg, 0.28 mmol, 3.0 eq) and Pd (dppf) Cl₂ (13 mg, 0.02 mmol, 0.2 eq). After stirring for 2 h at 80° C. under a nitrogen atmosphere, the reaction was quenched with water (10 mL) and then extracted with EtOAc (3×10 mL). The organic layer was dried over anhydrous Na₂SO₄, filtered and the filtrate was concentrated under reduced pressure. The residue was purified by silica gel column chromatography (1:1 AcOEt: petroleum ether) to afford tert-butyl 4-[(3aR,4R,6R,6aS)-6-[4-amino-5-(1-benzylpyrazol-3-yl)pyrrolo[2,3-d]pyrimidin-7-yl]-2,2-dimethyl-tetrahydro-3aH-cyclopenta[d][1,3]dioxol-4-yl]piperidine-1-carboxylate 30b (31 mg, 54%) as a yellow solid. LCMS [XBridge C18, 50*3.0 mm, 3.5 um; Mobile Phase A: Water/5 mM NH4HCO₃, Mobile Phase B: Methanol, 10%-95% B-3 min, 1.00 mL/min, ES, m/z]: TR=2.34 min, ES m/z [M+H]⁺: 614.

### (1R,2S,3R,5R)-3-(4-amino-5-(1-benzyl-1H-pyrazol-3-yl)-7H-pyrrolo[2,3-d]pyrimidin-7-yl)-5-(piperidin-4-yl)cyclopentane-1,2-diol (489)

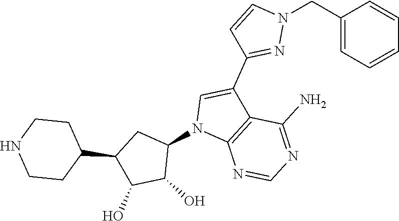 [SMILES: Nc1ncnc2c1c(-c1ccn(Cc3ccccc3)n1)cn2[C@@H]1C[C@H](C2CCNCC2)[C@@H](O)[C@H]1O]

Into a 8 mL vial were added tert-butyl 4-[(3aR,4R,6R,6aS)-6-[4-amino-5-(1-benzylpyrazol-3-yl)pyrrolo[2,3-d]pyrimidin-7-yl]-2,2-dimethyl-tetrahydro-3aH-cyclopenta[d][1,3]dioxol-4-yl]piperidine-1-carboxylate 30b (31 mg, 0.05 mmol, 1.0 eq) and TFA (2.0 mL) at room temperature. The resulting mixture was stirred for 1 h at 50° C. then concentrated under reduced pressure. The mixture was basified to pH>8 with NH3: H₂O. The crude product was purified by Prep-HPLC [Column: YMC-Actus Triart C18 ExRS, 30*150 mm, 5 um; Mobile Phase A: Water (10 mmol/L NH4HCO₃+0.1% NH3·H₂O), Mobile Phase B: ACN; Flow rate: 30 mL/min; Gradient: 20% B to 50% B in 8 min; Detector, UV 220&254 nm]to afford (1R,2S,3R,5R)-3-[4-amino-5-(1-benzylpyrazol-3-yl)pyrrolo[2,3-d]pyrimidin-7-yl]-5-(piperidin-4-yl)cyclopentane-1,2-diol 489 (9 mg, 37%) as an off-white solid. ¹H NMR (400 MHZ, DMSO-d₆+D₂O) δ 7.99 (s, 1H), 7.87 (d, J=2.3 Hz, 1H), 7.77 (s, 1H), 7.40-7.32 (m, 2H), 7.32-7.24 (m, 3H), 6.66 (d, J=2.3 Hz, 1H), 5.33 (s, 2H), 4.85-4.74 (m, 1H), 4.12 (dd, J=9.1, 6.4 Hz, 1H), 3.77 (t, J=5.2 Hz, 1H), 2.94-2.86 (m, 2H), 2.44-2.34 (m, 2H), 2.07-1.96 (m, 1H), 1.81-1.74 (m, 1H), 1.68-1.62 (m, 1H), 1.54-1.50 (m, 2H), 1.40-1.31 (m, 1H), 1.10-1.05 (m, 2H). LCMS [ZORBAX SB-Aq, 50*4.6 mm, 1.8 um; Mobile Phase A: Water/0.02% TFA, Mobile Phase B: Acetonitrile, 5%-40%-95% B-5 min, 1.50 mL/min, ES, m/z]: TR=2.31 min, ES m/z [M+H]⁺: 474. Chiral-SFC [Cellulose SB 50*3.0 mm,3.0 μm, Solvent B: MeOH (0.2% MSA), Oven Temperature: 35° C., Total Flow: 2.00 mL/min. Gradient 30% to 50% in 2.0 min, hold 1.0 min at 50%]: TR=1.91 min, single peak.

### Synthesis of (15,2R,3R,5R)-3-(piperidin-4-yl)-5-(6,7,8,9-tetrahydro-2H-2,3,5,6-tetraazabenzo[cd]azulen-2-yl)cyclopentane-1,2-diol (490)

### tert-butyl 4-((3aR,4R,6R,6aS)-2,2-dimethyl-6-(6,7,8,9-tetrahydro-2H-2,3,5,6-tetraazabenzo[cd]azulen-2-yl)tetrahydro-4H-cyclopenta[d][1,3]dioxol-4-yl)piperidine-1-carboxylate (31b)

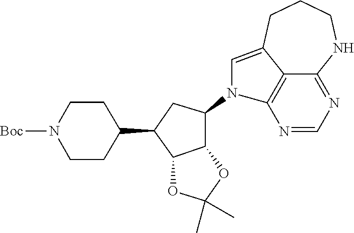 [SMILES: CC(C)(C)OC(=O)N1CCC([C@H]2C[C@@H](n3cc4c5c(ncnc53)NCCC4)[C@@H]3OC(C)(C)O[C@@H]32)CC1]

Tert-butyl 4-((3aR,4R,6S,6aR)-2,2-dimethyl-6-(((trifluoromethyl) sulfonyl)oxy)tetrahydro-4H-cyclopenta[d][1,3]dioxol-4-yl)piperidine-1-carboxylate (60 mg, 0.13 mmol, 1.0 eq) and 6,7,8,9-tetrahydro-2H-2,3,5,6-tetraazabenzo[cd]azulene (27 mg, 0.16 mmol, 1.2 eq) [WO2020050406]in DMF (4.0 mL) was added 60% NaH (21 mg, 0.52 mmol, 4.0 eq) in portions at 0° C. The resulting mixture was stirred for 1 h at room temperature. The reaction was quenched with water (10 mL) and then extracted with EtOAc (3×10 mL). The organic layer was dried over anhydrous Na₂SO₄, filtered and the filtrate was concentrated under reduced pressure. The residue was purified by silica gel column chromatography (1:1 AcOEt: Petroleum ether) to afford tert-butyl 4-((3aR,4R,6R,6aS)-2,2-dimethyl-6-(6,7,8,9-tetrahydro-2H-2,3,5,6-tetraazabenzo[cd]azulen-2-yl)tetrahydro-4H-cyclopenta[d][1,3]dioxol-4-yl)piperidine-1-carboxylate 31b (32 mg, 51%) as a yellow solid. LCMS [L-column3 ODS, 50*3.0 mm, 3.0 um; Mobile Phase A: Water/5 mM NH4HCO₃+0.05% Ammonia, Mobile Phase B: Acetonitrile/5% water, 5%-100% B-3 min, 1.20 mL/min, ES, m/z]: TR=2.00 min, ES m/z [M+H]⁺: 498.

### (1S,2R,3R,5R)-3-(piperidin-4-yl)-5-(6,7,8,9-tetrahydro-2H-2,3,5,6-tetraazabenzo[cd]azulen-2-yl)cyclopentane-1,2-diol (490)

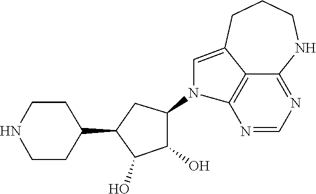 [SMILES: O[C@@H]1[C@H](O)[C@@H](C2CCNCC2)C[C@H]1n1cc2c3c(ncnc31)NCCC2]

Tert-butyl 4-((3aR,4R,6R,6aS)-2,2-dimethyl-6-(6,7,8,9-tetrahydro-2H-2,3,5,6-tetraazabenzo[cd]azulen-2-yl)tetrahydro-4H-cyclopenta[d][1,3]dioxol-4-yl)piperidine-1-carboxylate 31b (32 mg, 51%) was dissolved in 4M HCl in MeOH (2.0 mL). The resulting mixture was stirred for 1 h at 50° C., then concentrated under reduced pressure. The mixture was basified to pH>8 with NH3·H₂O. The crude product was purified by Prep-HPLC [Column: YMC-Actus Triart C18 ExRS, 30*150 mm, 5 um; Mobile Phase A: Water (10 mmol/L NH4HCO₃+0.1% NH3·H₂O), Mobile Phase B: ACN; Flow rate: 30 mL/min; Gradient: 20% B to 45% B in 8 min; Detector, UV 220&254 nm]to afford (1S,2R,3R,5R)-3-(piperidin-4-yl)-5-(6,7,8,9-tetrahydro-2H-2,3,5,6-tetraazabenzo[cd]azulen-2-yl)cyclopentane-1,2-diol 490 (13 mg, 57%) as an off-white solid. LCMS [ZORBAX SB-Aq, 50*4.6 mm, 1.8 μm; Mobile Phase A: Water/0.02% TFA, Mobile Phase B: Acetonitrile, 5%-95% B-3 min, 1.50 mL/min, ES, m/z]: TR=0.88 min, ES m/z [M+H]⁺: 538. ¹H NMR (300 MHz, DMSO-d₆+D₂O) δ 7.96 (s, 1H), 7.05 (s, 1H), 4.78-4.69 (m, 1H), 4.09-4.03 (m, 1H), 3.94-3.76 (m, 1H), 3.37-3.31 (m, 2H), 2.97-2.91 (m, 2H), 2.83-2.77 (m, 2H), 2.15-1.70 (m, 2H), 1.70-1.24 (m, 1H), 1.15-1.09 (m, 2H).

### Synthesis of (1R,2S,3R,5R)-3-(5-(1-benzyl-1H-pyrazol-3-yl)-2-chloro-7H-pyrrolo[2,3-d]pyrimidin-7-yl)-5-(1-methylpiperidin-4-yl)cyclopentane-1,2-diol (491)

### 5-(1-Benzyl-1H-pyrazol-3-yl)-2-chloro-74-pyrrolo[2,3-d]pyrimidine (67b)

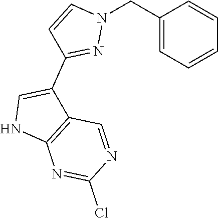 [SMILES: Clc1ncc2c(-c3ccn(Cc4ccccc4)n3)c[nH]c2n1]

Into a 40 mL vial were added 1-benzyl-3-(4,4,5,5-tetramethyl-1,3,2-dioxaborolan-2-yl)pyrazole (0.28 g, 1.0 mmol, 1.0 eq) [WO2017040450], 5-bromo-2-chloro-7H-pyrrolo[2,3-d]pyrimidine (0.27 g, 1.2 mmol, 1.2 eq), Pd (dtbpf) Cl₂ (65.0 mg, 0.1 mmol, 0.1 eq), K₃PO₄ (0.63 mg, 3.0 mmol, 3.0 eq), 1,4-dioxane (3.0 mL) and H₂O (0.3 mL) under N₂ atmosphere. The reaction was stirred at 90° C. for 2 h. The mixture was allowed to cool to room temperature, then concentrated under reduced pressure. The residue was dissolved in H₂O (10.0 mL) and the resulting mixture was extracted with AcOEt (3×10.0 mL). The combined organic layers were washed with brine and dried over anhydrous Na₂SO₄. After filtration, the filtrate was concentrated under reduced pressure. The residue was dissolved in CH₃CN (3.0 mL). The crude product was purified by reverse phase flash with the following conditions (Column: Welch Utimate AQ-C18, 50*250 mm*10 μm; Mobile Phase A: Water (0.05% NH4HCO₃), Mobile Phase B: ACN; Flow rate: 80 mL/min; Gradient: 5% B to 60% B in 15 min, 60% B; Wavelength: 220 nm) to afford 5-(1-benzyl-1H-pyrazol-3-yl)-2-chloro-7H-pyrrolo[2,3-d]pyrimidine 67b (150 mg, 48%) as a light yellow solid. LCMS (conditions L-column3 ODS, 50*3.0 mm, 3.0 μm, Mobile Phase A: Water/5 mM NH4HCO₃+0.05% Ammonia, Mobile Phase B: Acetonitrile/5% water, 2%-100% B-3 min, 1.50 L/min): TR=1.46 min; ES, m/z [M+H]⁺: 310, 312.

### Tert-butyl 4-((3aR,4R,6R,6aS)-6-(5-(1-benzyl-1H-pyrazol-3-yl)-2-chloro-7H-pyrrolo[2,3-d]pyrimidin-7-yl)-2,2-dimethyltetrahydro-4H-cyclopenta[d][1,3]dioxol-4-yl)piperidine-1-carboxylate (68b)

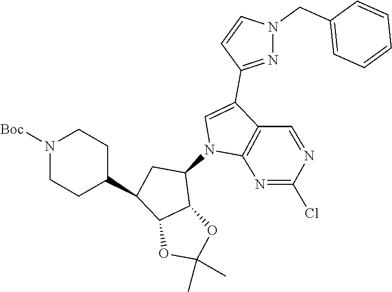 [SMILES: CC(C)(C)OC(=O)N1CCC([C@H]2C[C@@H](n3cc(-c4ccn(Cc5ccccc5)n4)c4cnc(Cl)nc43)[C@@H]3OC(C)(C)O[C@@H]32)CC1]

To a stirred solution of 5-(1-benzyl-1H-pyrazol-3-yl)-2-chloro-7H-pyrrolo[2,3-d]pyrimidine 67b (0.15 g, 0.5 mmol, 1.0 eq), tert-butyl 4-[(3aR,4R,6S,6aS)-6-hydroxy-2,2-dimethyl-tetrahydro-3aH-cyclopenta[d][1,3]dioxol-4-yl]piperidine-1-carboxylate 3 (0.20 g, 0.6 mmol, 1.2 eq) and PPh₃ (0.25 g, 1.0 mmol, 2.0 eq) in toluene (3.0 mL) was added DBAD (0.22 g, 1.0 mmol, 2.0 eq) in toluene (2.0 mL) dropwise at 0° C. The reaction was stirred at 50° C. for 2 h. The mixture was allowed to cool to room temperature, then concentrated under reduced pressure. The residue was dissolved in H₂O (10.0 mL) and the resulting mixture was extracted with AcOEt (3×10.0 mL). The combined organic layers were washed with brine and dried over anhydrous Na₂SO₄. After filtration, the filtrate was concentrated under reduced pressure. The residue was dissolved in DCM (3.0 mL) and purified by silica gel column chromatography (50:50 Petroleum ether/THF) to afford tert-butyl 4-((3aR,4R,6R,6aS)-6-(5-(1-benzyl-1H-pyrazol-3-yl)-2-chloro-7H-pyrrolo[2,3-d]pyrimidin-7-yl)-2,2-dimethyltetrahydro-4H-cyclopenta[d][1,3]dioxol-4-yl)piperidine-1-carboxylate 68b (100 mg, 33%) as a light yellow solid. LCMS (conditions L-column3 ODS, 50*3.0 mm, 3.0 μm, Mobile Phase Water/5 mM NH4HCO₃+0.05% Ammonia, Mobile Phase B: Acetonitrile/5% water, 2%-100% B-3 min, 1.50 L/min): TR=2.00 min; ES, m/z [M+H]⁺: 633, 635.

### (1R,2S,3R,5R)-3-[5-(1-benzylpyrazol-3-yl)-2-chloropyrrolo[2,3-d]pyrimidin-7-yl]-5-(piperidin-4-yl)cyclopentane-1,2-diol (69b)

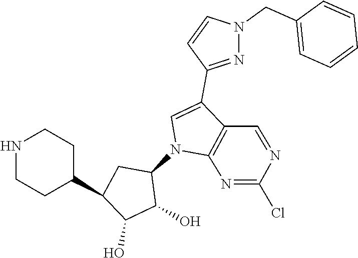 [SMILES: O[C@@H]1[C@H](O)[C@@H](C2CCNCC2)C[C@H]1n1cc(-c2ccn(Cc3ccccc3)n2)c2cnc(Cl)nc21]

Into a 40 mL vial were added tert-butyl 4-((3aR,4R,6R,6aS)-6-(5-(1-benzyl-1H-pyrazol-3-yl)-2-chloro-7H-pyrrolo[2,3-d]pyrimidin-7-yl)-2,2-dimethyltetrahydro-4H-cyclopenta[d][1,3]dioxol-4-yl)piperidine-1-carboxylate 68b (0.10 g, 0.2 mmol, 1.0 eq) and 4M HCl in MeOH (3.0 mL). The reaction was stirred at 40° C. for 20 min. The resulting mixture was concentrated under reduced pressure. The crude product (1R,2S,3R,5R)-3-(5-(1-benzyl-1H-pyrazol-3-yl)-2-chloro-7H-pyrrolo[2,3-d]pyrimidin-7-yl)-5-(piperidin-4-yl)cyclopentane-1,2-diol (70 mg) was used in the next step directly without further purification. LCMS (conditions L-column3 ODS, 50*3.0 mm, 3.0 μm, Mobile Phase A: Water/5 mM NH4HCO₃+0.05% Ammonia, Mobile Phase B: Acetonitrile/5% water, 2%-100% B-2.5 min, 1.50 L/min): TR=1.23 min; ES, m/z [M+H]⁺: 493, 495.

### (1R,2S,3R,5R)-3-(5-(1-benzyl-1H-pyrazol-3-yl)-2-chloro-7H-pyrrolo[2,3-d]pyrimidin-7-yl)-5-(1-methylpiperidin-4-yl)cyclopentane-1,2-diol (491)

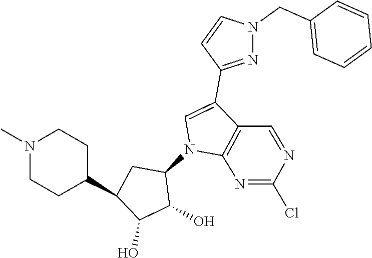 [SMILES: CN1CCC([C@H]2C[C@@H](n3cc(-c4ccn(Cc5ccccc5)n4)c4cnc(Cl)nc43)[C@H](O)[C@@H]2O)CC1]

Into a 40 mL vial were added (1R,2S,3R,5R)-3-[5-(1-benzylpyrazol-3-yl)-2-chloropyrrolo[2,3-d]pyrimidin-7-yl]-5-(piperidin-4-yl)cyclopentane-1,2-diol 69b (70 mg, 0.14 mmol, 1.0 eq), HCHO (13.0 mg, 0.4 mmol, 3.0 eq), NaBH (OAc) 3 (60.0 mg, 0.3 mmol, 2.0 eq), DIEA (55.0 mg, 0.5 mmol, 3.0 eq) and DCM (3.0 mL), and the reaction was stirred at room temperature for 2 h. The resulting mixture was concentrated under reduced pressure. The residue was dissolved in MeOH (3.0 mL) and was purified by reverse phase flash with the following conditions (Column: Welch Utimate AQ-C18, 50*250 mm*10 μm; Mobile Phase A: Water (10 mmol/L NH4HCO₃+0.1% NH3·H₂O), Mobile Phase B: ACN; Flow rate: 80 mL/min; Gradient: 5% B to 65% B in 15 min, 65% B; Wavelength: 220 nm) to afford (1R,2S,3R,5R)-3-(5-(1-benzyl-1H-pyrazol-3-yl)-2-chloro-7H-pyrrolo[2,3-d]pyrimidin-7-yl)-5-(1-methylpiperidin-4-yl)cyclopentane-1,2-diol (491)(19.5 mg, 30%) as a white solid. LCMS (Cortecs C18+, 50*3.0 mm, 2.7 μm, Mobile Phase A: Water/0.02% TFA, Mobile Phase B: Acetonitrile, 5%-95% B-3 min, 1.50 L/min): TR=0.98 min; ES, m/z [M+H]⁺: 507, 509. ¹H NMR (300 MHz, DMSO-d₆+D₂O) δ 9.24 (s, 1H), 8.22 (s, 1H), 7.90 (d, J=2.3 Hz, 1H), 7.40-7.22 (m, 5H), 6.73 (d, J=2.3 Hz, 1H), 5.40 (s, 2H), 4.98-4.82 (m, 1H), 4.14 (dd, J=9.4, 6.2 Hz, 1H), 3.78 (dd, J=6.3, 3.1 Hz, 1H), 2.76 (t, J=10.2 Hz, 2H), 2.12 (s, 3H), 2.10-2.03 (m, 1H), 1.81 (s, 3H), 1.71-1.47 (m, 3H), 1.25 (s, 3H).

### Synthesis of (1R,2S,3R,5R)-3-(4-amino-2-chloro-5-(1-(phenylsulfonyl)-1H-pyrazol-3-yl)-7H-pyrrolo[2,3-d]pyrimidin-7-yl)-5-(1-methylpiperidin-4-yl)cyclopentane-1,2-diol (492)

### Tert-butyl 4-((3aR,4R,6R,6aS)-6-(5-bromo-2,4-dichloro-7H-pyrrolo[2,3-d]pyrimidin-7-yl)-2,2-dimethyltetrahydro-4H-cyclopenta[d][1.3]dioxol-4-yl)piperidine-1-carboxylate (70b)

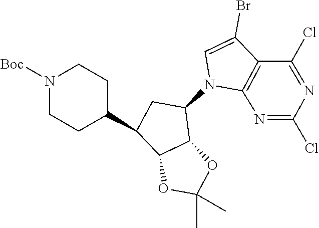 [SMILES: CC(C)(C)OC(=O)N1CCC([C@H]2C[C@@H](n3cc(Br)c4c(Cl)nc(Cl)nc43)[C@@H]3OC(C)(C)O[C@@H]32)CC1]

Into a 40 mL vial, was placed a solution of tert-butyl 4-((3aR,4R,6S,6aS)-6-hydroxy-2,2-dimethyltetrahydro-4H-cyclopenta[d][1,3]dioxol-4-yl)piperidine-1-carboxylate (1.53 g, 4.5 mmol, 1.5 eq) in toluene (4.0 mL). 5-Bromo-2,4-dichloro-7H-pyrrolo[2,3-d]pyrimidine (0.80 g, 3.0 mmol, 1.0 eq) and PPh₃ (1.57 g, 6.0 mmol, 2.0 eq) were added. The mixture was cooled to 0° C. To this was added DBAD (1.38 g, 6.0 mmol, 2.0 eq) in toluene (4.0 mL) dropwise over 1 min at 0° C. The resulting mixture was stirred for additional 20 min at 0° C., then 4 h at 50° C. The reaction was concentrated under reduced pressure. The residue was dissolved in DCM (2.0 mL) and was purified by silica gel column chromatography (78:22 Petroleum ether: AcOEt) to afford tert-butyl 4-((3aR,4R,6R,6aS)-6-(5-bromo-2,4-dichloro-7H-pyrrolo[2,3-d]pyrimidin-7-yl)-2,2-dimethyltetrahydro-4H-cyclopenta[d][1,3]dioxol-4-yl)piperidine-1-carboxylate 70b (1.0 g, 56%) as an off-white solid. LCMS (L-column3 ODS, 50*3.0 mm, 3.0 μm, Mobile Phase Water/5 mM NH4HCO₃+0.05% Ammonia, Mobile Phase B: Acetonitrile/5% water, 2%-100% B-3 min, 1.50 L/min): T_{R}=2.52 min, ES m/z [M+H]: 589, 591.

### (1R,2S,3R,5R)-3-(5-bromo-2,4-dichloro-7H-pyrrolo[2,3-d]pyrimidin-7-yl)-5-(piperidin-4-yl)cyclopentane-1,2-diol (71b)

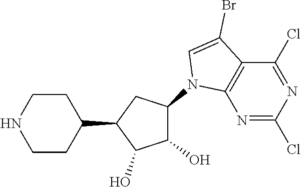 [SMILES: O[C@@H]1[C@H](O)[C@@H](C2CCNCC2)C[C@H]1n1cc(Br)c2c(Cl)nc(Cl)nc21]

Into a 40 mL vial, was placed a solution of tert-butyl 4-((3aR,4R,6R,6aS)-6-(5-bromo-2,4-dichloro-7H-pyrrolo[2,3-d]pyrimidin-7-yl)-2,2-dimethyltetrahydro-4H-cyclopenta[d][1,3]dioxol-4-yl)piperidine-1-carboxylate 70b (0.30 g, 0.5 mmol, 1.0 eq) in 4M HCl in MeOH (3.0 mL). The resulting was stirred for 20 min at 60° C. The resulting mixture was concentrated under vacuum to afford crude product (1R,2S,3R,5R)-3-(5-bromo-2,4-dichloro-7H-pyrrolo[2,3-d]pyrimidin-7-yl)-5-(piperidin-4-yl)cyclopentane-1,2-diol 71b (300 mg) as a light yellow solid. LCMS (L-column3 ODS, 50*3.0 mm, 3.0 μm, Mobile Phase Water/5 mM NH4HCO₃+0.05% Ammonia, Mobile Phase B: Acetonitrile/5% water, 2%-100% B-3 min, 1.50 L/min): T_{R}=1.32 min, ES m/z [M+H]⁺: 449, 451.

### 5-Bromo-2,4-dichloro-7-((3aS,4R,6R,6aR)-2,2-dimethyl-6-(1-methylpiperidin-4-yl)tetrahydro-4H-cyclopenta[d][1,3]dioxol-4-yl)-7H-pyrrolo[2,3-d]pyrimidine (72b)

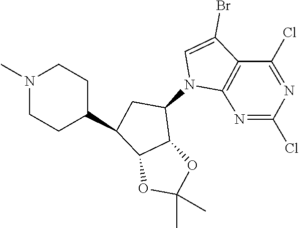 [SMILES: CN1CCC([C@H]2C[C@@H](n3cc(Br)c4c(Cl)nc(Cl)nc43)[C@@H]3OC(C)(C)O[C@@H]32)CC1]

Into a 40 ml vial, was placed a solution of (1R,2S,3R,5R)-3-(5-bromo-2,4-dichloro-7H-pyrrolo[2,3-d]pyrimidin-7-yl)-5-(piperidin-4-yl)cyclopentane-1,2-diol 71b (0.23 g, 0.8 mmol, 1.0 eq) in DCM (2.0 mL). 30% HCHO in H₂O (0.24 g, 2.5 mmol, 5.0 eq), DIEA (130.92 mg, 1.0 mmol, 2.0 eq) and NaBH (OAc) 3 (0.21 g, 1.0 mmol, 2.0 eq) were added. The mixture was stirred for 1 h at room temperature. The resulting mixture was concentrated under reduced pressure and dissolved in acetone (1.0 mL). 2,2-Dimethoxypropane (1.0 mL) and p-TsOH (0.44 mg, 2.5 mmol, 5.0 eq) were added. The mixture was stirred for 2 h at room temperature. The reaction was quenched by the addition of saturated aqueous NaHCO₃ at room temperature. The resulting mixture was extracted with AcOEt (3×5.0 mL). The combined organic layers were washed with brine and dried over anhydrous Na₂SO₄. After filtration, the filtrate was concentrated under reduced pressure. The residue was purified by silica gel column chromatography, (10:90 MeOH: DCM) to afford 5-bromo-2,4-dichloro-7-((3aS,4R,6R,6aR)-2,2-dimethyl-6-(1-methylpiperidin-4-yl)tetrahydro-4H-cyclopenta[d][1,3]dioxol-4-yl)-7H-pyrrolo[2,3-d]pyrimidine 72b (110 mg, 42%) as a light yellow solid. LCMS (L-column3 ODS, 50*3.0 mm, 3.0 μm, Mobile Phase A: Water/5 mM NH4HCO₃+0.05% Ammonia, Mobile Phase B: Acetonitrile/5% water, 2%-100% B-3 min, 1.50 L/min): T_{R}=2.17 min, ES m/z [M+H]⁺: 503, 505.

### 5-Bromo-2-chloro-7-((3aS,4R,6R,6aR)-2,2-dimethyl-6-(1-methylpiperidin-4-yl)tetrahydro-4H-cyclopenta[d][1,3]dioxol-4-yl)-7H-pyrrolo[2,3-d]pyrimidin-4-amine (73b)

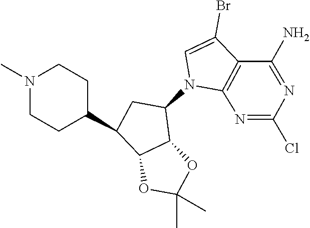 [SMILES: CN1CCC([C@H]2C[C@@H](n3cc(Br)c4c(N)nc(Cl)nc43)[C@@H]3OC(C)(C)O[C@@H]32)CC1]

Into an 8 mL vial, was placed a solution of 5-bromo-2,4-dichloro-7-((3aS,4R,6R,6aR)-2,2-dimethyl-6-(1-methylpiperidin-4-yl)tetrahydro-4H-cyclopenta[d][1,3]dioxol-4-yl)-7H-pyrrolo[2,3-d]pyrimidine 72b (0.11 g, 0.2 mmol, 1.0 eq) in 7M NH3/MeOH (3.0 mL). The mixture was stirred for 6 h at 80° C. The resulting solution was concentrated under vacuum to afford 5-bromo-2-chloro-7-((3aS,4R,6R,6aR)-2,2-dimethyl-6-(1-methylpiperidin-4-yl)tetrahydro-4H-cyclopenta[d][1,3]dioxol-4-yl)-7H-pyrrolo[2,3-d]pyrimidin-4-amine 73b (120 mg, crude) as a light yellow solid. This was directly used in next step without purification. LCMS (L-column3 ODS, 50*3.0 mm, 3.0 μm, Mobile Phase Water/5 mM NH4HCO₃+0.05% Ammonia, Mobile Phase B: Acetonitrile/5% water, 2%-100% B-3 min, 1.50 L/min): T_{R}=1.69 min, ES m/z [M+H]⁺: 484, 486.

### 2-Chloro-7-((3aS,4R,6R,6aR)-2,2-dimethyl-6-(1-methylpiperidin-4-yl)tetrahydro-4H-cyclopenta[d][1,3]dioxol-4-yl)-5-(1-(phenylsulfonyl)-1H-pyrazol-3-yl)-7H-pyrrolo[2,3-d]pyrimidin-4-amine (74b)

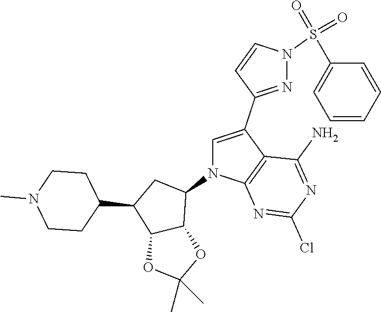 [SMILES: CN1CCC([C@H]2C[C@@H](n3cc(-c4ccn(S(=O)(=O)c5ccccc5)n4)c4c(N)nc(Cl)nc43)[C@@H]3OC(C)(C)O[C@@H]32)CC1]

Into a 40 mL vial, was placed a solution of 5-bromo-2-chloro-7-((3aS,4R,6R,6aR)-2,2-dimethyl-6-(1-methylpiperidin-4-yl)tetrahydro-4H-cyclopenta[d][1,3]dioxol-4-yl)-7H-pyrrolo[2,3-d]pyrimidin-4-amine 73b (0.10 g, 0.2 mmol, 1.0 eq) in dioxane (0.9 mL) and H₂O (0.1 mL). K₃PO₄ (87.56 mg, 0.4 mmol, 2.0 eq), 1-(benzenesulfonyl)pyrazol-3-ylboronic acid (67.58 mg, 0.3 mmol, 1.3 eq) and Pd (dtbpf) Cl₂ (13.44 mg, 0.021 mmol, 0.1 eq) were added. The resulting mixture was stirred for 2 h at 80° C. under N₂ atmosphere. The reaction was concentrated under reduced pressure. The residue was purified by Prep-HPLC (Column: Welch Ultimate XB-C18, 50*250 mm, 10 μm; Mobile Phase A: 0.1% HCl, Mobile Phase B: ACN; Flow rate: 90 mL/min; Gradient: 5% B-5% B-2 min, 5% B-50% B-12 min) to afford 2-chloro-7-((3aS,4R,6R,6aR)-2,2-dimethyl-6-(1-methylpiperidin-4-yl)tetrahydro-4H-cyclopenta[d][1,3]dioxol-4-yl)-5-(1-(phenylsulfonyl)-1H-pyrazol-3-yl)-7H-pyrrolo[2,3-d]pyrimidin-4-amine 74b (30 mg, 24%) as a light brown solid. LCMS (L-column3 ODS, 50*3.0 mm, 3.0 μm, Mobile Phase Water/5 mM NH4HCO₃+0.05% Ammonia, Mobile Phase B: Acetonitrile/5% water, 2%-100% B-3 min, 1.50 L/min): T_{R}=1.93 min, ES m/z [M+H]⁺: 612, 614.

### (1R,2S,3R,5R)-3-(4-Amino-2-chloro-5-(1-(phenylsulfonyl)-1H-pyrazol-3-yl)-7H-pyrrolo[2,3-d]pyrimidin-7-yl)-5-(1-methylpiperidin-4-yl)cyclopentane-1,2-diol (492)

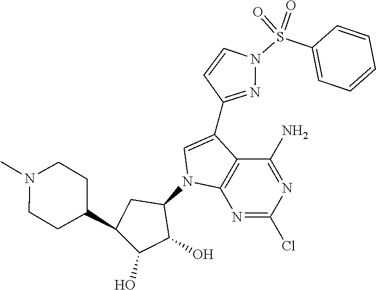 [SMILES: CN1CCC([C@H]2C[C@@H](n3cc(-c4ccn(S(=O)(=O)c5ccccc5)n4)c4c(N)nc(Cl)nc43)[C@H](O)[C@@H]2O)CC1]

Into a 40 mL vial, was placed a solution of 2-chloro-7-((3aS,4R,6R,6aR)-2,2-dimethyl-6-(1-methylpiperidin-4-yl)tetrahydro-4H-cyclopenta[d][1,3]dioxol-4-yl)-5-(1-(phenylsulfonyl)-1H-pyrazol-3-yl)-7H-pyrrolo[2,3-d]pyrimidin-4-amine 74b (30 mg, 0.049 mmol, 1.0 eq) in TFA (2.0 mL). The mixture was stirred for 1 h at room temperature. The resulting mixture was concentrated under reduced pressure. The residue was dissolved in MeOH (2.0 mL) and basified to pH>8 with TEA. The mixture was purified by Prep-HPLC (Column: YMC-Actus Triart C18, 30*150 mm, 5 μm; Mobile Phase A: Water (10 mmol/L NH4HCO₃+0.1% NH3·H₂O), Mobile Phase B: ACN; Flow rate: 30 ml/min; Gradient: 24% B to 49% B in 7 min) to afford (1R,2S,3R,5R)-3-(4-amino-2-chloro-5-(1-(phenylsulfonyl)-1H-pyrazol-3-yl)-7H-pyrrolo[2,3-d]pyrimidin-7-yl)-5-(1-methylpiperidin-4-yl)cyclopentane-1,2-diol 492 (4.8 mg, 17%) as an off-white solid. LCMS (ZORBAX SB-Aq, 50*4.6 mm, 1.8 μm, Mobile Phase A: Water/0.02% TFA, Mobile Phase B: Acetonitrile, 2%-100% B-3 min, 1.50 L/min): T_{R}=1.45 min, ES m/z [M+H]⁺: 572, 574. ¹H NMR (400 MHz, DMSO-d₆) δ8.70 (br, s, 1H), 8.55 (d, J=2.9 Hz, 1H), 8.10 (s, 1H), 7.97 (d, J=7.9 Hz, 2H), 7.88 (br, s, 1H), 7.84-7.76 (m, 1H), 7.73-7.60 (m, 2H), 7.09 (d, J=2.9 Hz, 1H), 4.81 (d, J=6.6 Hz, 1H), 4.78-4.68 (m, 1H), 4.59 (d, J=4.8 Hz, 1H), 4.05 (q, J=6.8 Hz, 1H), 3.79-3.68 (m, 1H), 2.84-2.71 (m, 2H), 2.13 (s, 3H), 2.08-1.97 (m, 1H), 1.88-1.71 (m, 3H), 1.71-1.62 (m, 1H), 1.59-1.51 (m, 1H), 1.51-1.39 (m, 1H), 1.24-1.10 (m, 3H).

### Synthesis of (1R,2S,3R,5R)-3-(4-amino-2-chloro-5-(thiazol-2-yl)-7H-pyrrolo[2,3-d]pyrimidin-7-yl)-5-(1-methyl-1,2,3,6-tetrahydropyridin-4-yl)cyclopentane-1,2-diol (493)

### Tert-butyl 4-((3aR,4R,6aR)-2,2-dimethyl-6-oxotetrahydro-4H-cyclopenta[d][1,3]dioxol-4-yl)-3,6-dihydropyridine-1 (2H)-carboxylate (75b)

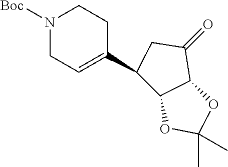 [SMILES: CC(C)(C)OC(=O)N1CC=C([C@H]2CC(=O)[C@@H]3OC(C)(C)O[C@H]23)CC1]

Into a 40 mL vial were added (3aR,6aR)-2,2-dimethyl-3aH,6aH-cyclopenta[d][1,3]dioxol-4-one (1.5 g, 9.7 mmol, 1.0 eq), 1-(tert-butoxycarbonyl)-3,6-dihydro-2H-pyridin-4-ylboronic acid (0.33 g, 14.6 mmol, 1.5 eq), cyclooctadiene rhodium chloride dimer (0.72 g, 1.5 mmol, 0.15 eq), aqueous KOH (660 μL, 3M) and CH₃OH (10.0 mL) under N₂ atmosphere. The reaction was irradiated with microwave radiation for 2 h at 40° C. The mixture was allowed to cool and was concentrated under reduced pressure. The residue was dissolved in DCM (5.0 mL) and purified by silica gel column chromatography (70:30 Petroleum ether/THF) to afford tert-butyl 4-((3aR,4R,6aR)-2,2-dimethyl-6-oxotetrahydro-4H-cyclopenta[d][1,3]dioxol-4-yl)-3,6-dihydropyridine-1 (2H)-carboxylate 75b (1.0 g, 31%) as a yellow oil. LCMS (conditions: L-column3 C18, 50*3.0 mm, 3.0 μm, Mobile Phase A: Water/5 mM NH4HCO₃; Mobile Phase B: Methanol, 2%-100% B-2.5 min, 1.50 L/min): T_{R}=1.73 min, ES m/z [M+H]⁺: 338.

### Tert-butyl 4-((3aR,4R,6S,6aS)-6-hydroxy-2,2-dimethyltetrahydro-4H-cyclopenta[d][1,3]dioxol-4-yl)-3,6-dihydropyridine-1 (2H)-carboxylate (76b)

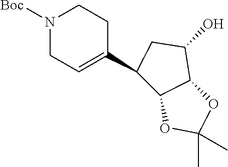 [SMILES: CC(C)(C)OC(=O)N1CC=C([C@H]2C[C@H](O)[C@@H]3OC(C)(C)O[C@@H]32)CC1]

To a stirred solution of tert-butyl 4-((3aR,4R,6aR)-2,2-dimethyl-6-oxotetrahydro-4H-cyclopenta[d][1,3]dioxol-4-yl)-3,6-dihydropyridine-1 (2H)-carboxylate 75b (0.70 g, 2.0 mmol, 1.0 eq) in MeOH (5.0 mL) was added NaBH₄ (95.0 mg, 2.5 mmol, 1.2 eq) in portions at 0° C. The reaction was stirred at room temperature for 20 min, then concentrated under reduced pressure. The residue was dissolved in DCM (5.0 mL) and purified by silica gel column chromatography (70:30 Petroleum ether/THF) to afford tert-butyl 4-((3aR,4R,6S,6aS)-6-hydroxy-2,2-dimethyltetrahydro-4H-cyclopenta[d][1,3]dioxol-4-yl)-3,6-dihydropyridine-1 (2H)-carboxylate 76b (600 mg, 85%) as a yellow oil. LCMS (conditions: L-column3 C18, 50*3.0 mm, 3.0 μm, Mobile Phase A: Water/5 mM NH4HCO₃; Mobile Phase B: Methanol, 2%-100% B-2.5 min, 1.50 L/min): T_{R}=1.60 min; ES m/z [M−H]: 338.

### Tert-butyl 4-((3aR,4R,6R,6aS)-6-(2,4-dichloro-5-iodo-7H-pyrrolo[2,3-d]pyrimidin-7-yl)-2,2-dimethyltetrahydro-4H-cyclopenta[d][1,3]dioxol-4-yl)-3,6-dihydropyridine-1 (2H)-carboxylate (77b)

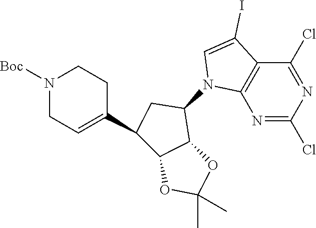 [SMILES: CC(C)(C)OC(=O)N1CC=C([C@H]2C[C@@H](n3cc(I)c4c(Cl)nc(Cl)nc43)[C@@H]3OC(C)(C)O[C@@H]32)CC1]

To a stirred solution of tert-butyl 4-((3aR,4R,6S,6aS)-6-hydroxy-2,2-dimethyltetrahydro-4H-cyclopenta[d][1,3]dioxol-4-yl)-3,6-dihydropyridine-1 (2H)-carboxylate 76 (0.56 g, 1.6 mmol, 1.3 eq), 2,4-dichloro-5-iodo-7H-pyrrolo[2,3-d]pyrimidine (0.4 g, 1.3 mmol, 1.0 eq) and PPh₃ 0.67 g, 2.6 mmol, 2.0 eq) in toluene (4.0 mL) was added DBAD (0.58 g, 2.5 mmol, 2.0 eq) in toluene (4.0 mL) dropwise at 0° C. The reaction was stirred at 50° C. for 3 h. The mixture was allowed to cool to room temperature, then concentrated under reduced pressure. The residue was dissolved in DCM (6.0 mL) and was purified by silica gel column chromatography (60:40 Petroleum ether/THF) to afford tert-butyl 4-((3aR,4R,6R,6aS)-6-(2,4-dichloro-5-iodo-7H-pyrrolo[2,3-d]pyrimidin-7-yl)-2,2-dimethyltetrahydro-4H-cyclopenta[d][1,3]dioxol-4-yl)-3,6-dihydropyridine-1 (2H)-carboxylate 77b (400 mg, 49%) as a white solid. LCMS (conditions L-column3 ODS, 50*3.0 mm, 3.0 μm, Mobile Phase A: Water/5 mM NH4HCO₃+0.05% Ammonia, Mobile Phase B: Acetonitrile, 2%-100% B-2.5 min), 1.50 L/min): T_{R}=2.0 min, ES m/z [M+H]⁺: 635, 637.

### Tert-butyl 4-((3aR,4R,6R,6aS)-6-(4-amino-2-chloro-5-iodo-7H-pyrrolo[2,3-d]pyrimidin-7-yl)-2,2-dimethyltetrahydro-4H-cyclopenta[d][1,3]dioxol-4-yl)-3,6-dihydropyridine-1 (2H)-carboxylate (78b)

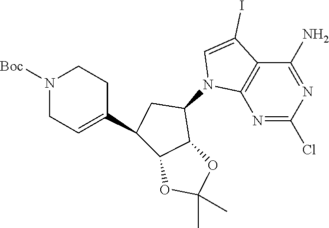 [SMILES: CC(C)(C)OC(=O)N1CC=C([C@H]2C[C@@H](n3cc(I)c4c(N)nc(Cl)nc43)[C@@H]3OC(C)(C)O[C@@H]32)CC1]

Into a 40 mL vial were added tert-butyl 4-((3aR,4R,6R,6aS)-6-(2,4-dichloro-5-iodo-7H-pyrrolo[2,3-d]pyrimidin-7-yl)-2,2-dimethyltetrahydro-4H-cyclopenta[d][1,3]dioxol-4-yl)-3,6-dihydropyridine-1 (2H)-carboxylate 77b (0.39 g, 0.6 mmol, 1.0 eq) and 7M NH3 in MeOH (6.0 mL). The reaction was stirred at 80° C. for 3 h. The mixture was allowed to cool to room temperature, then concentrated under reduced pressure. The residue was dissolved in DCM (5.0 mL) and purified by silica gel column chromatography (50:50 Petroleum ether/THF) to afford tert-butyl 4-((3aR,4R,6R,6aS)-6-(4-amino-2-chloro-5-iodo-7H-pyrrolo[2,3-d]pyrimidin-7-yl)-2,2-dimethyltetrahydro-4H-cyclopenta[d][1,3]dioxol-4-yl)-3,6-dihydropyridine-1 (2H)-carboxylate 78b (350 mg, 92%) as a yellow oil. LCMS (conditions Cortecs C18+, 50*3.0 mm, 2.7 μm, Mobile Phase A: Water/0.05% TFA, Mobile Phase B: Acetonitrile, 2%-95% B-3 min, 1.50 L/min): T_{R}=1.84 min, ES m/z [M+H]⁺: 616, 618.

### Tert-butyl 4-((3aR,4R,6R,6aS)-6-(4-amino-2-chloro-5-(thiazol-2-yl)-7H-pyrrolo[2,3-d]pyrimidin-7-yl)-2,2-dimethyltetrahydro-4H-cyclopenta[d][1,3]dioxol-4-yl)-3,6-dihydropyridine-1 (2H)-carboxylate (79b)

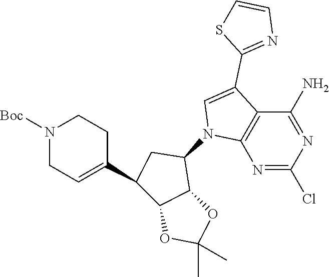 [SMILES: CC(C)(C)OC(=O)N1CC=C([C@H]2C[C@@H](n3cc(-c4nccs4)c4c(N)nc(Cl)nc43)[C@@H]3OC(C)(C)O[C@@H]32)CC1]

Into a 40 mL vial were added tert-butyl 4-((3aR,4R,6R,6aS)-6-(4-amino-2-chloro-5-iodo-7H-pyrrolo[2,3-d]pyrimidin-7-yl)-2,2-dimethyltetrahydro-4H-cyclopenta[d][1,3]dioxol-4-yl)-3,6-dihydropyridine-1 (2H)-carboxylate 78b (0.32 g, 0.52 mmol, 1.0 eq), (tributylstannyl)-1,3-thiazole (0.39 g, 1.0 mmol, 2.0 eq), Pd (PPh₃) 2Cl₂ (37.0 mg, 0.052 mmol, 0.1 eq) and toluene (4.0 mL). The reaction was stirred at 120° C. for 3 h. The mixture was allowed to cool to room temperature, then concentrated under reduced pressure. The residue was dissolved in DCM (5.0 mL) and purified by silica gel column chromatography (50:50 Petroleum ether/THF) to afford tert-butyl 4-[(3aR,4R,6R,6aS)-6-[4-amino-2-chloro-5-(1,3-thiazol-2-yl)pyrrolo[2,3-d]pyrimidin-7-yl]-2,2-dimethyl-tetrahydro-3aH-cyclopenta[d][1,3]dioxol-4-yl]-3,6-dihydro-2H-pyridine-1-carboxylate 79b (200 mg, 67%) as a white solid. LCMS (conditions Proshell HPH-C18, 50*3.0 mm, 2.7 μm, Mobile Phase A: Water (Water/5 mM NH4HCO₃+0.05% Ammonia), Mobile Phase B: Acetonitrile, 5%-100% B-2.5 min, 1.50 L/min): T_{R}=1.93 min, ES m/z [M+H]⁺: 573, 575.

### (1R,2S,3R,5R)-3-(4-Amino-2-chloro-5-(thiazol-2-yl)-7H-pyrrolo[2,3-d]pyrimidin-7-yl)-5-(1,2,3,6-tetrahydropyridin-4-yl)cyclopentane-1,2-diol (80b)

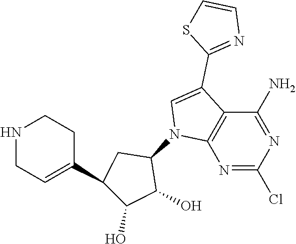 [SMILES: Nc1nc(Cl)nc2c1c(-c1nccs1)cn2[C@@H]1C[C@H](C2=CCNCC2)[C@@H](O)[C@H]1O]

Into a 40 mL vial were added tert-butyl 4-[(3aR,4R,6R,6aS)-6-[4-amino-2-chloro-5-(1,3-thiazol-2-yl)pyrrolo[2,3-d]pyrimidin-7-yl]-2,2-dimethyl-tetrahydro-3aH-cyclopenta[d][1,3]dioxol-4-yl]-3,6-dihydro-2H-pyridine-1-carboxylate 79b (0.18 g, 0.3 mmol, 1.0 eq) and 4M HCl in MeOH (4.0 mL). The reaction mixture was stirred for 30 min at 40° C. The mixture was allowed to cool to room temperature, then concentrated under reduced pressure to afford (1R,2S,3R,5R)-3-[4-amino-2-chloro-5-(1,3-thiazol-2-yl)pyrrolo[2,3-d]pyrimidin-7-yl]-5-(1,2,3,6-tetrahydropyridin-4-yl)cyclopentane-1,2-diol 80b (120 mg, crude) which was used in the next step directly without further purification. LCMS (conditions Proshell HPH-C18, 50*3.0 mm, 2.7 μm, Mobile Phase A: Water (Water/5 mM NH4HCO₃+0.05% Ammonia), Mobile Phase B: Acetonitrile, 5%-100% B-2.5 min, 1.50 L/min): T_{R}=1.23 min, ES m/z [M+H]⁺: 433, 435

### (1R,2S,3R,5R)-3-(4-Amino-2-chloro-5-(thiazol-2-yl)-7H-pyrrolo[2,3-d]pyrimidin-7-yl)-5-(1-methyl-1,2,3,6-tetrahydropyridin-4-yl)cyclopentane-1,2-diol (493)

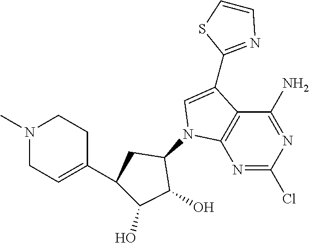 [SMILES: CN1CC=C([C@H]2C[C@@H](n3cc(-c4nccs4)c4c(N)nc(Cl)nc43)[C@H](O)[C@@H]2O)CC1]

Into a 40 mL vial were added (1R,2S,3R,5R)-3-[4-amino-2-chloro-5-(1,3-thiazol-2-yl)pyrrolo[2,3-d]pyrimidin-7-yl]-5-(1,2,3,6-tetrahydropyridin-4-yl)cyclopentane-1,2-diol 79 (40 mg, 0.092 mmol, 1.0 eq), 30% HCHO in H₂O (9.2 mg, 0.092 mmol, 1.0 eq), NaBH (OAc) 3 (40.0 mg, 0.184 mmol, 2.0 eq), DIEA (24.0 mg, 0.184 mmol, 2.0 eq) and DCM (2.0 mL). The mixture was stirred at room temperature for 2 h, then concentrated under reduced pressure. The residue was dissolved in CH₃CN (2.0 mL) and purified by reverse phase flash with the following conditions (Column: YMC-Actus Triart C18, 30*150 mm, 5 μm; Mobile Phase A: Water (10 mmol/L NH4HCO₃+0.1% NH3·H₂O), Mobile Phase B: ACN; Flow rate: 30 mL/min; Gradient: 24% B to 60% B in 10 min, 60% B; Wavelength: 220 nm) to afford (1R,2S,3R,5R)-3-(4-amino-2-chloro-5-(thiazol-2-yl)-7H-pyrrolo[2,3-d]pyrimidin-7-yl)-5-(1-methyl-1,2,3,6-tetrahydropyridin-4-yl)cyclopentane-1,2-diol 493 (7.7 mg, 18%) as a white solid. LCMS (ZORBAX SB-Aq, 50*4.6 mm, 1.8 μm, Mobile Phase A: Water/0.02% TFA, Mobile Phase B: Acetonitrile, 5%-95% B-3 min, 1.50 L/min): T_{R}=1.27 min, ES m/z [M+H]⁺: 447, 449. ¹H NMR (400 MHZ, DMSO-d₆) δ9.55 (br, s, 1H), 8.24 (s, 1H), 7.97 (br, s, 1H), 7.84 (d, J=3.4 Hz, 1H), 7.66 (d, J=3.4 Hz, 1H), 5.57 (br, s, 1H), 4.96-4.93 (m, 1H), 4.83-4.68 (m, 2H), 4.32-4.15 (m, 1H), 3.89-3.86 (m, 1H), 3.01-2.92 (s, 2H), 2.82-2.67 (m, 2H), 2.64-2.50 (m, 2H), 2.30-2.26 (m, 3H), 2.13-2.10 (m, 2H), 1.94-1.82 (m, 1H).

### Synthesis of (1R,2S,3R,5R)-3-(4-amino-2-chloro-5-(thiazol-2-yl)-7H-pyrrolo[2,3-d]pyrimidin-7-yl)-5-(1-phenethyl-1,2,3,6-tetrahydropyridin-4-yl)cyclopentane-1,2-diol. (494)

### (1R,2S,3R,5R)-3-(4-Amino-2-chloro-5-(thiazol-2-yl)-7H-pyrrolo[2,3-d]pyrimidin-7-yl)-5-(1-phenethyl-1,2,3,6-tetrahydropyridin-4-yl)cyclopentane-1,2-diol (494)

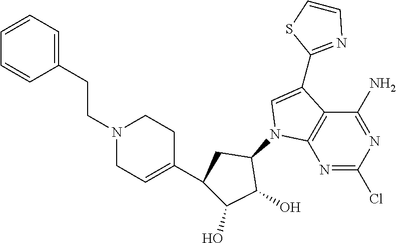 [SMILES: Nc1nc(Cl)nc2c1c(-c1nccs1)cn2[C@@H]1C[C@H](C2=CCN(CCc3ccccc3)CC2)[C@@H](O)[C@H]1O]

Into a 40 mL vial were added (1R,2S,3R,5R)-3-[4-amino-2-chloro-5-(1,3-thiazol-2-yl)pyrrolo[2,3-d]pyrimidin-7-yl]-5-(1,2,3,6-tetrahydropyridin-4-yl)cyclopentane-1,2-diol 80b (60 mg, 0.14 mmol, 1.0 eq), phenylacetaldehyde (17.0 mg, 0.14 mmol, 1.0 eq), NaBH (OAc) 3 (59.0 mg, 0.28 mmol, 2.0 eq), DIEA (37.0 mg, 0.28 mmol, 2.0 eq) and DCM (2.0 mL) at room temperature. The mixture was stirred at room temperature for 2 h, then concentrated under reduced pressure. The residue was dissolved in CH₃CN (3.0 mL) and purified by Prep-HPLC with the following conditions (Column: YMC-Actus Triart C18, 30*150 mm, 5 μm; Mobile Phase A: Water (10 mmol/L NH4HCO₃+0.1% NH3·H₂O), Mobile Phase B: ACN; Flow rate: 30 mL/min; Gradient: 24% B to 65% B in 10 min, 65% B; Wavelength: 220 nm) to afford (1R,2S,3R,5R)-3-(4-amino-2-chloro-5-(thiazol-2-yl)-7H-pyrrolo[2,3-d]pyrimidin-7-yl)-5-(1-phenethyl-1,2,3,6-tetrahydropyridin-4-yl)cyclopentane-1,2-diol 494 (21.9 mg, 29%) as a white solid. LCMS (Cortecs C18+, 50*3.0 mm, 2.7 μm, Mobile Phase A: Water/0.02% TFA, Mobile Phase B: Acetonitrile, 5%-95% B-3 min, 1.50 L/min): T_{R}=1.03 min, ES m/z [M+H]⁺: 537, 539. ¹H NMR (300 MHZ, DMSO-d₆) δ9.55 (br, s, 1H), 8.25 (s, 1H), 7.97 (br, s, 1H), 7.84 (d, J=3.4 Hz, 1H), 7.66 (d, J=3.4 Hz, 1H), 7.32-7.09 (m, 5H), 5.57 (s, 1H), 4.92 (d, J=6.4 Hz, 1H), 4.80-7.78 (m, 2H), 4.21 (q, J=7.2 Hz, 1H), 3.92-3.84 (m, 1H), 3.00 (s, 2H), 2.81-2.72 (m, 2H), 2.61-2.56 (m, 3H), 2.55-2.51 (m, 2H), 2.27-2.21 (m, 1H), 2.11-2.05 (m, 2H), 1.96-1.79 (m, 1H).

### Synthesis of (1R,2S,3R,5R)-3-(2-Chloro-5-(1-(phenylsulfonyl)-1H-pyrazol-3-yl)-7H-pyrrolo[2,3-d]pyrimidin-7-yl)-5-(1-methylpiperidin-4-yl)cyclopentane-1,2-diol. (495)

### Tert-butyl 4-((3aR,4R,6R,6aS)-6-(5-bromo-2-chloro-7H-pyrrolo[2,3-d]pyrimidin-7-yl)-2,2-dimethyltetrahydro-4H-cyclopenta[d][1,3]dioxol-4-yl)piperidine-1-carboxylate (90b)

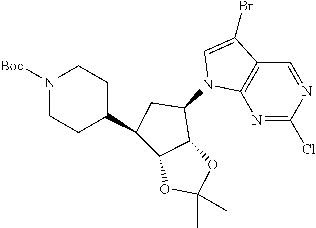 [SMILES: CC(C)(C)OC(=O)N1CCC([C@H]2C[C@@H](n3cc(Br)c4cnc(Cl)nc43)[C@@H]3OC(C)(C)O[C@@H]32)CC1]

A solution of tert-butyl 4-[(3aR,4R,6S,6aS)-6-hydroxy-2,2-dimethyl-tetrahydro-3aH-cyclopenta[d][1,3]dioxol-4-yl]piperidine-1-carboxylate (0.19 g, 0.6 mmol, 1.3 eq) in toluene (15.0 mL) was treated with 5-bromo-2-chloro-7H-pyrrolo[2,3-d]pyrimidine (0.10 g, 0.4 mmol, 1.0 eq) and PPh₃ (0.25 g, 0.8 mmol, 2.0 eq), followed by the addition of DBAD (0.20 g, 0.8 mmol, 2.0 eq) dropwise at 0° C. The reaction was stirred for 2 h at 50° C. The resulting mixture was concentrated under reduced pressure. The residue was purified by silica gel column chromatography (60:40 Petroleum ether/AcOEt) to afford tert-butyl 4-((3aR,4R,6R,6aS)-6-(5-bromo-2-chloro-7H-pyrrolo[2,3-d]pyrimidin-7-yl)-2,2-dimethyltetrahydro-4H-cyclopenta[d][1,3]dioxol-4-yl)piperidine-1-carboxylate 90b (150 mg, 62%) as a white solid. LCMS (conditions: XBridge C18, 50*3.0 mm, 3.5 μm, Mobile Phase A: Water/5 mM NH4HCO₃, Mobile Phase B: Methanol, 2%-100% B-2 min, 1.50 L/min), T_{R}=1.62 min, ES m/z [M+H]⁺: 555, 557.

### (1R,2S,3R,5R)-3-(5-bromo-2-chloro-7H-pyrrolo[2,3-d]pyrimidin-7-yl)-5-(piperidin-4-yl)cyclopentane-1,2-diol (91b)

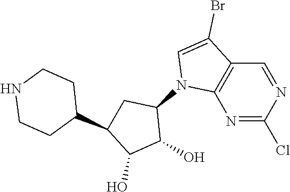 [SMILES: O[C@@H]1[C@H](O)[C@@H](C2CCNCC2)C[C@H]1n1cc(Br)c2cnc(Cl)nc21]

Into an 8 mL vial were added 4-((3aR,4R,6R,6aS)-6-(5-bromo-2-chloro-7H-pyrrolo[2,3-d]pyrimidin-7-yl)-2,2-dimethyltetrahydro-4H-cyclopenta[d][1,3]dioxol-4-yl)piperidine-1-carboxylate 90b (0.14 g, 0.3 mmol, 1.0 eq) and 4M HCl in MeOH (4.0 mL) at room temperature. The resulting mixture was stirred for 30 min at 40° C. The reaction was concentrated under reduced pressure. The residue was purified by silica gel column chromatography (10:90 MeOH/DCM) to afford to afford (1R,2S,3R,5R)-3-(5-bromo-2-chloro-7H-pyrrolo[2,3-d]pyrimidin-7-yl)-5-(piperidin-4-yl)cyclopentane-1,2-diol 91b (76 mg, 72%) as a white solid. LCMS (conditions: XBridge C18, 50*3.0 mm, 3.5 μm, Mobile Phase A: Water/5 mM NH4HCO₃, Mobile Phase B: Methanol, 2%-100% B-2 min, 1.50 L/min), T_{R}=1.20 min, ES m/z [M+H]⁺: 415, 417.

### (1R,2S,3R,5R)-3-(5-bromo-2-chloro-7H-pyrrolo[2,3-d]pyrimidin-7-yl)-5-(1-methylpiperidin-4-yl)cyclopentane-1,2-diol (92b)

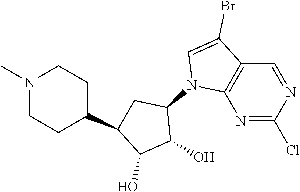 [SMILES: CN1CCC([C@H]2C[C@@H](n3cc(Br)c4cnc(Cl)nc43)[C@H](O)[C@@H]2O)CC1]

Into a 8 mL vial were added (1R,2S,3S,5R)-3-{5-bromo-2-chloropyrrolo[2,3-d]pyrimidin-7-yl}-5-(piperidin-4-yl)cyclopentane-1,2-diol 91b (71 mg, 0.2 mmol, 1.0 eq), HCHO (35.0 mg, 0.34 mmol, 2.0 eq), DIEA (45.0 mg, 0.34 mmol, 2.0 eq) and NaBH (OAc) 3 (73.0 mg, 0.34 mmol, 2.0 eq) in 3.0 mL DCM. The resulting mixture was stirred for 1 h then concentrated under reduced pressure. The residue was purified by silica gel column chromatography (50:50 Petroleum ether/AcOEt) to afford (1R,2S,3R,5R)-3-(5-bromo-2-chloro-7H-pyrrolo[2,3-d]pyrimidin-7-yl)-5-(1-methylpiperidin-4-yl)cyclopentane-1,2-diol 92b (53 mg, 72%) as a white solid. LCMS (conditions L-column3 ODS, 50*3.0 mm, 3.0 μm, Mobile Phase A: Water/5 mM NH4HCO₃+0.05% Ammonia, Mobile Phase B: Acetonitrile, 5%-100% B-2 min, 1.50 L/min): T_{R}=1.20 min, ES m/z [M+H]⁺: 429, 431.

### 5-Bromo-2-chloro-7-((3aS,4R,6R,6aR)-2,2-dimethyl-6-(1-methylpiperidin-4-yl)tetrahydro-4H-cyclopenta[d][1,3]dioxol-4-yl)-7H-pyrrolo[2,3-d]pyrimidine (93b)

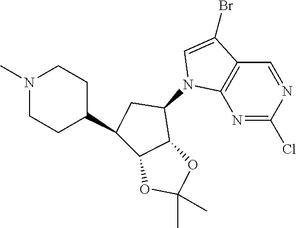 [SMILES: CN1CCC([C@H]2C[C@@H](n3cc(Br)c4cnc(Cl)nc43)[C@@H]3OC(C)(C)O[C@@H]32)CC1]

Into a 40 mL vial were added (1R,2S,3R,5R)-3-{5-bromo-2-chloropyrrolo[2,3-d]pyrimidin-7-yl}-5-(1-methylpiperidin-4-yl)cyclopentane-1,2-diol 92b (50.0 mg, 0.1 mmol, 1.0 eq), 2,2-dimethoxypropane (1.0 mL, 0.12 mmol), p-TsOH (32.0 mg, 0.2 mmol, 2.0 eq) and acetone (1.0 mL). The reaction was stirred at room temperature for 2 h. The resulting mixture was concentrated under reduced pressure. The residue was dissolved in DCM (2.0 mL) and purified by silica gel column chromatography (70:30 Petroleum ether/AcOEt) to afford 5-bromo-2-chloro-7-((3aS,4R,6R,6aR)-2,2-dimethyl-6-(1-methylpiperidin-4-yl)tetrahydro-4H-cyclopenta[d][1,3]dioxol-4-yl)-7H-pyrrolo[2,3-d]pyrimidine 93b (40 mg, 80%) as a white solid. LCMS (conditions: Proshell Kinetex XB-C18, 50*3.0 mm, 2.7 μm, Mobile Phase A: Water (0.05% TFA), Mobile Phase B: Acetonitrile, 2%-100% B-2 min, 1.50 L/min), T_{R}=0.75 min, ES m/z [M+H]⁺: 469, 471.

### 2-Chloro-7-((3aS,4R,6R,6aR)-2,2-dimethyl-6-(1-methylpiperidin-4-yl)tetrahydro-4H-cyclopenta[d][1,3]dioxol-4-yl)-5-(1-(phenylsulfonyl)-1H-pyrazol-3-yl)-7H-pyrrolo[2,3-d]pyrimidine (94b)

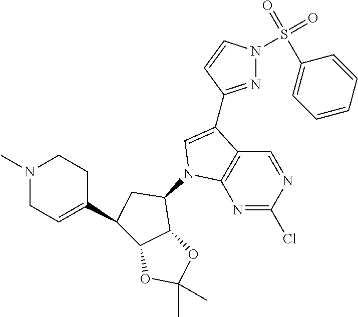 [SMILES: CN1CC=C([C@H]2C[C@@H](n3cc(-c4ccn(S(=O)(=O)c5ccccc5)n4)c4cnc(Cl)nc43)[C@@H]3OC(C)(C)O[C@@H]32)CC1]

Into a 8 mL vial were added 5-bromo-2-chloro-7-((3aS,4R,6R,6aR)-2,2-dimethyl-6-(1-methylpiperidin-4-yl)tetrahydro-4H-cyclopenta[d][1,3]dioxol-4-yl)-7H-pyrrolo[2,3-d]pyrimidine 93b (40 mg, 0.085 mmol, 1.0 eq), 1-(benzenesulfonyl)pyrazol-3-ylboronic acid (25.7 mg, 0.10 mmol, 1.2 eq), Pd (dtbpf) Cl₂ (11.0 mg, 0.017 mmol, 0.2 eq), K₃PO₄ (54.2 mg, 0.26 mmol, 3.0 eq), dioxane (2.0 mL) and H₂O (0.2 mL) under N₂ atmosphere, The reaction was stirred at 90° C. for 3 h. The mixture was allowed to cool to room temperature, then concentrated under reduced pressure. The residue was dissolved in H₂O (10.0 mL). The resulting mixture was extracted with EtOAc (2×20.0 mL). The combined organic layers were washed with brine and dried over anhydrous Na₂SO₄. After filtration, the filtrate was concentrated under reduced pressure. The residue was dissolved in DCM (2.0 mL).and purified by silica gel column chromatography (90:10 DCM/MeOH) to afford 2-chloro-7-((3aS,4R,6R,6aR)-2,2-dimethyl-6-(1-methylpiperidin-4-yl)tetrahydro-4H-cyclopenta[d][1,3]dioxol-4-yl)-5-(1-(phenylsulfonyl)-1H-pyrazol-3-yl)-7H-pyrrolo[2,3-d]pyrimidine 94b (20 mg, 39%) as a white solid. LCMS (conditions: Proshell HPH-C18, 50*3.0 mm, 2.7 μm, Mobile Phase A: Water (0.05% ammonia water), Mobile Phase B: Acetonitrile, 2%-100% B-2 min, 1.50 L/min): T_{R}=1.49 min, [M+H]⁺: 597, 599.

### (1R,2S,3R,5R)-3-(2-Chloro-5-(1-(phenylsulfonyl)-1H-pyrazol-3-yl)-7H-pyrrolo[2,3-d]pyrimidin-7-yl)-5-(1-methylpiperidin-4-yl)cyclopentane-1,2-diol (495)

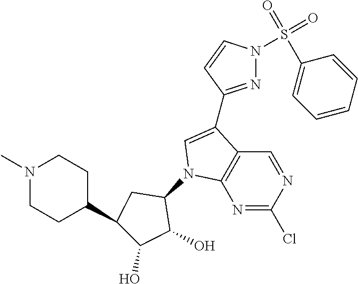 [SMILES: CN1CCC([C@H]2C[C@@H](n3cc(-c4ccn(S(=O)(=O)c5ccccc5)n4)c4cnc(Cl)nc43)[C@H](O)[C@@H]2O)CC1]

Into a 40 mL vial were added 4-[(3aR,4R,6R,6aS)-6-{5-[1-(benzenesulfonyl)pyrazol-3-yl]-2-chloropyrrolo[2,3-d]pyrimidin-7-yl}-2,2-dimethyl-tetrahydro-3aH-cyclopenta[d][1,3]dioxol-4-yl]-1-methylpiperidine 94b (20 mg, 0.033 mmol, 1.0 eq), TFA (1.5 mL) and DCM (0.5 mL). The reaction was stirred at 40° C. for 30 min. The mixture was allowed to cool to room temperature, then concentrated under reduced pressure. The residue was dissolved in CH₃CN (2.0 mL) and basified to pH>8 with TEA. The mixture was purified by reverse phase flash with the following conditions (Column: Welch Utimate AQ-C18, 50*250 mm*10 μm; Mobile Phase A: Water (10 mmol/L NH4HCO₃+0.1% NH3·H₂O), Mobile Phase B: ACN; Flow rate: 80 mL/min; Gradient: 5% B to 70% B in 15 min, 70% B; Wavelength: 220 nm) to afford (1R,2S,3R,5R)-3-(2-chloro-5-(1-(phenylsulfonyl)-1H-pyrazol-3-yl)-7H-pyrrolo[2,3-d]pyrimidin-7-yl)-5-(1-methylpiperidin-4-yl)cyclopentane-1,2-diol (8.6 mg, 48%) as a white solid. LCMS (Cortecs C18+, 50*3.0 mm, 2.7 μm, Mobile Phase A: Water/0.02% TFA, Mobile Phase B: Acetonitrile, 5%-95% B-3 min, 1.50 L/min): T_{R}=1.00 min, ES m/z [M+H]⁺: 557, 559. ¹H NMR (300 MHZ, DMSO-d₆) δ9.20 (s, 1H), 8.57 (d, J=2.9 Hz, 1H), 8.50 (s, 1H), 8.14-8.01 (m, 2H), 7.85-7.75 (m, 1H), 7.72-7.63 (m, 2H), 7.10 (d, J=2.7 Hz, 1H), 5.00-4.85 (m, 2H), 4.71-4.61 (m, 1H), 4.16-4.04 (m, 1H), 3.80-3.77 (m, 1H), 2.86-2.67 (m, 2H), 2.14 (s, 3H), 2.13-2.06 (m, 1H), 1.88-1.67 (m, 4H), 1.66-1.47 (m, 2H), 1.30-1.21 (m, 3H).

### Synthesis of (1R,2S,3R,5R)-3-(5-(4-benzylthiazol-2-yl)-2-chloro-7H-pyrrolo[2,3-d]pyrimidin-7-yl)-5-(1-methylpiperidin-4-yl)cyclopentane-1,2-diol (496)

### Tert-butyl 4-[(3aR,4R,6R,6aS)-6-{2-chloro-5-iodopyrrolo[2,3-d]pyrimidin-7-yl}-2,2-dimethyl-tetrahydro-3aH-cyclopenta[d][1,3]dioxol-4-yl]piperidine-1-carboxylate (95b)

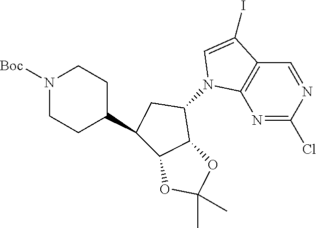 [SMILES: CC(C)(C)OC(=O)N1CCC([C@H]2C[C@H](n3cc(I)c4cnc(Cl)nc43)[C@@H]3OC(C)(C)O[C@@H]32)CC1]

A solution of tert-butyl 4-[(3aR,4R,6S,6aS)-6-hydroxy-2,2-dimethyl-tetrahydro-3aH-cyclopenta[d][1,3]dioxol-4-yl]piperidine-1-carboxylate 3 (350 mg, 1.03 mmol, 1.0 eq), PPh₃ (538 mg, 2.05 mmol, 2.0 eq) in toluene (10 mL) was treated with 2-chloro-5-iodo-7H-pyrrolo[2,3-d]pyrimidine (286 mg, 1.02 mmol, 1.0 eq) at 0° C. To the above mixture was added DBAD (472 mg, 2.05 mmol, 2.0 eq) in toluene (1.0 mL) dropwise over 5 min at 0° C. The resulting mixture was stirred for additional 2 h at 60° C. The reaction was concentrated under reduced pressure. The residue was purified by silica gel column chromatography (4:1 Petroleum ether: THF) to afford tert-butyl 4-[(3aR,4R,6R,6aS)-6-{2-chloro-5-iodopyrrolo[2,3-d]pyrimidin-7-yl}-2,2-dimethyl-tetrahydro-3aH-cyclopenta[d][1,3]dioxol-4-yl]piperidine-1-carboxylate 95b (330 mg, 53%) as a white solid. LCMS ((Cortecs C18+, 50*3.0 mm, 2.7 μm, Mobile Phase A: Water/5 mM NH4HCO₃+0.05% Ammonia, Mobile Phase B: Acetonitrile, 5%-100% B-3.0 min, 1.50 L/min): T_{R}=2.41 min, ES m/z [M+H]⁺: 603, 605.

### Tert-butyl 4-((3aR,4R,6R,6aS)-6-(5-(4-benzylthiazol-2-yl)-2-chloro-7H-pyrrolo[2,3-d]pyrimidin-7-yl)-2,2-dimethyltetrahydro-4H-cyclopenta[d][1,3]dioxol-4-yl)piperidine-1-carboxylate (96b)

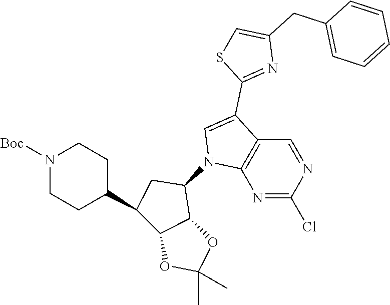 [SMILES: CC(C)(C)OC(=O)N1CCC([C@H]2C[C@@H](n3cc(-c4nc(Cc5ccccc5)cs4)c4cnc(Cl)nc43)[C@@H]3OC(C)(C)O[C@@H]32)CC1]

To a stirred solution of tert-butyl 4-[(3aR,4R,6R,6aS)-6-{2-chloro-5-iodopyrrolo[2,3-d]pyrimidin-7-yl}-2,2-dimethyl-tetrahydro-3aH-cyclopenta[d][1,3]dioxol-4-yl]piperidine-1-carboxylate 95b (330 mg, 0.55 mmol, 1.0 eq) and Pd (PPh₃) 2Cl₂ (76.84 mg, 0.11 mmol, 0.2 eq) in toluene (2.0 mL) was added (4-benzyl-1,3-thiazol-2-yl)(chloro) zinc (602.24 mg, 2.19 mmol, 4.0 eq) in THF (3.0 mL) dropwise. The reaction was stirred at 60° C. for 4 h. The mixture was allowed to cool to room temperature, then quenched with saturated NH₄Cl solution. The resulting mixture was extracted with AcOEt (3×10.0 mL). The combined organic layers were washed with brine and dried over anhydrous Na₂SO₄. After filtration, the filtrate was concentrated under reduced pressure. The residue was purified by silica gel column chromatography (1:1 Petroleum ether: THF) to afford tert-butyl 4-[(3aR,4R,6R,6aS)-6-[5-(4-benzyl-1,3-thiazol-2-yl)-2-chloropyrrolo[2,3-d]pyrimidin-7-yl]-2,2-dimethyl-tetrahydro-3aH-cyclopenta[d][1,3]dioxol-4-yl]piperidine-1-carboxylate 96b (320 mg, 89%) as a clear oil. LCMS (Cortecs C18+, 50*3.0 mm, 2.7 μm, Mobile Phase A: Water/5 mM NH4HCO₃+0.05% Ammonia, Mobile Phase B: Acetonitrile, 5%-100% B-2.5 min, 1.50 L/min): T_{R}=2.56 min, ES m/z [M+H]⁺: 650, 652.

### (1R,2S,3R,5R)-3-[5-(4-benzyl-1,3-thiazol-2-yl)-2-chloropyrrolo[2,3-d]pyrimidin-7-yl]-5-(piperidin-4-yl)cyclopentane-1,2-diol (97b)

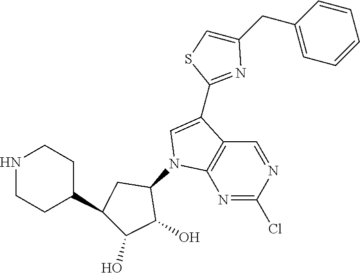 [SMILES: O[C@@H]1[C@H](O)[C@@H](C2CCNCC2)C[C@H]1n1cc(-c2nc(Cc3ccccc3)cs2)c2cnc(Cl)nc21]

Into a 40 mL vial were added tert-butyl 4-[(3aR,4R,6R,6aS)-6-[5-(4-benzyl-1,3-thiazol-2-yl)-2-chloropyrrolo[2,3-d]pyrimidin-7-yl]-2,2-dimethyl-tetrahydro-3aH-cyclopenta[d][1,3]dioxol-4-yl]piperidine-1-carboxylate 96b (320 mg, 0.5 mmol, 1.0 eq) and 4M HCl in MeOH (2.0 mL) at room temperature, The reaction was stirred at 60° C. for 30 min. The resulting mixture was concentrated under reduced pressure to afford (1R,2S,3R,5R)-3-[5-(4-benzyl-1,3-thiazol-2-yl)-2-chloropyrrolo[2,3-d]pyrimidin-7-yl]-5-(piperidin-4-yl)cyclopentane-1,2-diol HCl salt 97b (310 mg, crude) as a white solid, which was used in the next step directly without further purification. LCMS (Cortecs C18+, 50*3.0 mm, 2.7 μm, Mobile Phase A: Water/5 mM NH4HCO₃+0.05% Ammonia, Mobile Phase B: Acetonitrile, 5%-100% B-2.5 min, 1.50 L/min): T_{R}=1.52 min, ES m/z [M+H]⁺: 510, 512.

### (1R,2S,3R,5R)-3-(5-(4-benzylthiazol-2-yl)-2-chloro-7H-pyrrolo[2,3-d]pyrimidin-7-yl)-5-(1-methylpiperidin-4-yl)cyclopentane-1,2-diol (496)

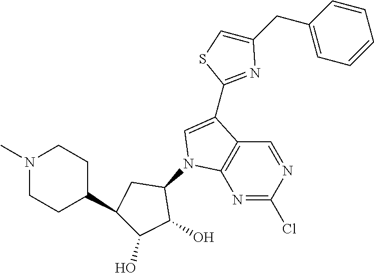 [SMILES: CN1CCC([C@H]2C[C@@H](n3cc(-c4nc(Cc5ccccc5)cs4)c4cnc(Cl)nc43)[C@H](O)[C@@H]2O)CC1]

Into a 40 ml vial were added (1R,2S,3R,5R)-3-[5-(4-benzyl-1,3-thiazol-2-yl)-2-chloropyrrolo[2,3-d]pyrimidin-7-yl]-5-(piperidin-4-yl)cyclopentane-1,2-diol HCl salt 97b (80 mg, 0.16 mmol, 1.0 eq), HCHO (9.42 mg, 0.31 mmol, 2.0 eq), AcOH (0.47 mg, 0.01 mmol, 0.05 eq) and DCM (5.0 ml) at 0° C. To this was added NaBH (OAc) 3 (66.5 mg, 0.31 mmol, 2.0 eq) in portions over 2 min at 0° C. The resulting mixture was stirred for 2 h, then concentrated under reduced pressure. The residue was dissolved in MeOH (2.0 mL) and was purified Prep-HPLC (Column: YMC-Actus Triart C18, 30*150 mm, 5 μm; Mobile Phase A: Water (10 mmol/L NH4HCO₃+0.1% NH3·H₂O), Mobile Phase B: ACN; Flow rate: 30 mL/min; Gradient: 20% B to 45% B in 7 min, 50% B; Wavelength: 220 nm) to afford (1R,2S,3R,5R)-3-[5-(4-benzyl-1,3-thiazol-2-yl)-2-chloropyrrolo[2,3-d]pyrimidin-7-yl]-5-(1-methylpiperidin-4-yl)cyclopentane-1,2-diol 496 (8.8 mg, 10%) as an off-white solid. LCMS (Cortecs C18+, 50*3.0 mm, 2.7 μm, Mobile Phase A: Water/0.02% TFA, Mobile Phase B: Acetonitrile, 5%-95% B-3 min, 1.50 L/min): T_{R}=1.09 min, ES m/z [M+H]⁺: 524, 526. ¹H NMR (300 MHz, DMSO-d₆) δ 9.31 (s, 1H), 8.61 (s, 1H), 7.43-7.16 (m, 6H), 4.97-4.86 (m, 2H), 4.67 (d, J=4.6 Hz, 1H), 4.28-4.17 (m, 1H), 4.15 (s, 2H), 3.82-3.76 (m, 1H), 3.00-2.78 (m, 2H), 2.34-2.17 (m, 3H), 2.17-1.97 (m, 3H), 1.93-1.84 (m, 1H), 1.78-1.57 (m, 3H), 1.36-1.19 (m, 3H). Chiral-HPLC [Column: CHIRALPAK IG-3, 100*4.6 mm, 3 μm IG30CS-UL011; Mobile Phase A: MtBE, Mobile Phase B: Methanol (0.2% MSA); Total Flow: 1.00 mL/min; Conc. of Pump B: 50.0%; Oven Temperature: 25 C]: T_{R}=1.55 min (5 min run), single peak.

### Synthesis of (1R,2S,3R,5R)-3-(5-(4-benzylthiazol-2-yl)-2-chloro-7H-pyrrolo[2,3-d]pyrimidin-7-yl)-5-(1-(cyclopropylmethyl)piperidin-4-yl)cyclopentane-1,2-diol (497)

### (1R,2S,3R,5R)-3-(5-(4-Benzylthiazol-2-yl)-2-chloro-7H-pyrrolo[2,3-d]pyrimidin-7-yl)-5-(1-(cyclopropylmethyl)piperidin-4-yl)cyclopentane-1,2-diol (497)

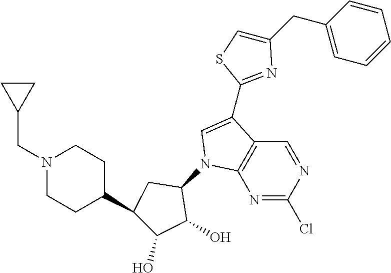 [SMILES: O[C@@H]1[C@H](O)[C@@H](C2CCN(CC3CC3)CC2)C[C@H]1n1cc(-c2nc(Cc3ccccc3)cs2)c2cnc(Cl)nc21]

Into a 40 mL vial were added (1R,2S,3R,5R)-3-[5-(4-benzyl-1,3-thiazol-2-yl)-2-chloropyrrolo[2,3-d]pyrimidin-7-yl]-5-(piperidin-4-yl)cyclopentane-1,2-diol 95b (80 mq, 0.15 mmol, 1.0 eq), cyclopropanecarbaldehyde (16.5 mg, 0.23 mmol, 1.5 eq), AcOH (0.47 mg, 0.01 mmol, 0.05 eq) and DCM (5.0 ml) at 0° C. To the above mixture was added NaBH (OAc) 3 (66.5 mg, 0.31 mmol, 2.0 eq) in portions over 2 min at 0° C. The resulting mixture was stirred for 2 h, then concentrated under reduced pressure. The residue was dissolved in MeOH (2.0 mL) and was purified by Prep-HPLC (Column: YMC-Actus Triart C18, 30*150 mm, 5 μm; Mobile Phase A: Water (10 mmol/L NH4HCO₃+0.1% NH3·H₂O), Mobile Phase B: ACN; Flow rate: 30 mL/min; Gradient: 20% B to 45% B in 7 min, 50% B; Wavelength: 220 nm) to afford (1R,2S,3R,5R)-3-(5-(4-benzylthiazol-2-yl)-2-chloro-7H-pyrrolo[2,3-d]pyrimidin-7-yl)-5-(1-(cyclopropylmethyl)piperidin-4-yl)cyclopentane-1,2-diol 497 (15.4 mg, 17%) as an off-white solid. LCMS (Cortecs C18+, 50*3.0 mm, 2.7 μm, Mobile Phase A: Water/0.02% TFA, Mobile Phase B: Acetonitrile, 5%-95% B-3 min, 1.50 L/min): T_{R}=1.16 min, ES m/z [M+H]⁺: 564, 566. ¹H NMR (300 MHZ, DMSO-d₆) δ 9.31 (s, 1H), 8.61 (s, 1H), 7.42-7.17 (m, 6H), 4.99-4.87 (m, 2H), 4.65 (d, J=4.5 Hz, 1H), 4.27-4.16 (m, 1H), 4.15 (s, 2H), 3.84-3.75 (m, 1H), 3.05-2.92 (m, 2H), 2.18-2.01 (m, 3H), 1.99-1.77 (m, 3H), 1.74-1.57 (m, 3H), 1.34-1.15 (m, 3H), 0.85-0.79 (m, 1H), 0.51-0.39 (m, 2H), 0.09-0.04 (m, 2H). Chiral-HPLC [Column: XA-RP-YMC Cellulose-SC, 4.6*100 mm, 3 μm, 119IA70110; Mobile Phase A: Water (0.05% FA), Mobile Phase B: Acetonitrile; Total Flow: 1.00 mL/min; Conc. of Pump B: 10.0%; Oven Temperature: 25 C]: T_{R}=4.40 min (10 min run), single peak.

### Synthesis of (1R,2S,3R,5R)-3-(5-(4-benzylthiazol-2-yl)-2-chloro-7H-pyrrolo[2,3-d]pyrimidin-7-yl)-5-(1-phenethylpiperidin-4-yl)cyclopentane-1,2-diol (498)

### (1R,2S,3R,5R)-3-(5-(4-Benzylthiazol-2-yl)-2-chloro-7H-pyrrolo[2,3-d]pyrimidin-7-yl)-5-(1-phenethylpiperidin-4-yl)cyclopentane-1,2-diol (498)

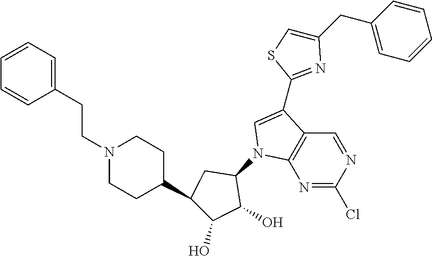 [SMILES: O[C@@H]1[C@H](O)[C@@H](C2CCN(CCc3ccccc3)CC2)C[C@H]1n1cc(-c2nc(Cc3ccccc3)cs2)c2cnc(Cl)nc21]

Into a 40 mL vial were added (1R,2S,3R,5R)-3-[5-(4-benzyl-1,3-thiazol-2-yl)-2-chloropyrrolo[2,3-d]pyrimidin-7-yl]-5-(piperidin-4-yl)cyclopentane-1,2-diol 95b (80 mq, 0.16 mmol, 1.0 eq), phenylacetaldehyde (28 mg, 0.24 mmol, 1.5 eq), AcOH (0.47 mg, 0.01 mmol, 0.05 eq) and DCM (5.0 ml) at 0° C. NaBH (OAc) 3 (66.5 mg, 0.31 mmol, 2.0 eq) was added in portions over 2 min at 0° C. The resulting mixture was stirred for 2h then concentrated under reduced pressure. The residue was dissolved in MeOH (2.0 mL). The mixture was purified by Prep-HPLC (Column: YMC-Actus Triart C18, 30*150 mm, 5 μm; Mobile Phase A: Water (10 mmol/L NH4HCO₃+0.1% NH3·H₂O), Mobile Phase B: ACN; Flow rate: 30 mL/min; Gradient: 20% B to 45% B in 7 min, 50% B; Wavelength: 220 nm) to afford (1R,2S,3R,5R)-3-[5-(4-benzyl-1,3-thiazol-2-yl)-2-chloropyrrolo[2,3-d]pyrimidin-7-yl]-5-[1-(2-phenylethyl)piperidin-4-yl]cyclopentane-1,2-diol 498 (16.9 mg, 17.54%) as an off-white solid. LCMS (Cortecs C18+, 50*3.0 mm, 2.7 μm, Mobile Phase A: Water/0.02% TFA, Mobile Phase B: Acetonitrile, 5%-95% B-3 min, 1.50 L/min): T_{R}=1.27 min, ES m/z [M+H]⁺: 614, 616. ¹H NMR (300 MHZ, DMSO-d₆) δ 9.31 (s, 1H), 8.61 (s, 1H), 7.42-7.12 (m, 11H), 5.01-4.88 (m, 2H), 4.69-4.63 (m, 1H), 4.21 (dd, J=9.4, 6.2 Hz, 1H), 4.15 (s, 2H), 3.80 (d, J=6.1 Hz, 1H), 3.04-2.86 (m, 2H), 2.79-2.68 (m, 2H), 2.49-2.43 (m, 2H), 2.19-2.01 (m, 1H), 1.98-1.79 (m, 3H), 1.76-1.54 (m, 3H), 1.48-1.24 (m, 3H).

### Synthesis of (1R,2S,3R,5R)-3-(5-(4-benzylthiazol-2-yl)-2-chloro-7H-pyrrolo[2,3-d]pyrimidin-7-yl)-5-(1-(2,2,2-trifluoroethyl)piperidin-4-yl)cyclopentane-1,2-diol (499)

### (1R,2S,3R,5R)-3-(5-(4-Benzylthiazol-2-yl)-2-chloro-7H-pyrrolo[2,3-d]pyrimidin-7-yl)-5-(1-(2,2,2-trifluoroethyl)piperidin-4-yl)cyclopentane-1,2-diol (499)

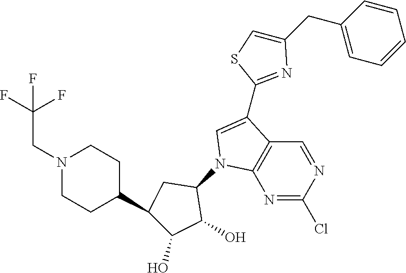 [SMILES: O[C@@H]1[C@H](O)[C@@H](C2CCN(CC(F)(F)F)CC2)C[C@H]1n1cc(-c2nc(Cc3ccccc3)cs2)c2cnc(Cl)nc21]

Into a 40 mL vial were added (1R,2S,3R,5R)-3-[5-(4-benzyl-1,3-thiazol-2-yl)-2-chloropyrrolo[2,3-d]pyrimidin-7-yl]-5-(piperidin-4-yl)cyclopentane-1,2-diol 95b (80 mq, 0.15 mmol, 1.0 eq), DIEA (61 mg, 0.47 mmol, 3.0 eq) and DCM (5 mL) at 0° C. 2,2,2-Trifluoroethyl trifluoromethanesulfonate (72.81 mq, 0.3 mmol, 2.0 eq) was added dropwise over 5 min at 0° C. The resulting mixture was stirred for 2h then concentrated under reduced pressure. The residue was dissolved in MeOH (2.0 mL) and was purified by Prep-HPLC (Column: YMC-Actus Triart C18, 30*150 mm, 5 μm; Mobile Phase A: Water (10 mmol/L NH4HCO₃+0.1% NH3·H₂O), Mobile Phase B: ACN; Flow rate: 30 mL/min; Gradient: 20% B to 45% B in 7 min, 50% B; Wavelength: 220 nm) to afford (1R,2S,3R,5R)-3-[5-(4-benzyl-1,3-thiazol-2-yl)-2-chloropyrrolo[2,3-d]pyrimidin-7-yl]-5-[1-(2,2,2-trifluoroethyl)piperidin-4-yl]cyclopentane-1,2-diol 499 (5.2 mg, 5%) as an off-white solid. LCMS (Cortecs C18+, 50*3.0 mm, 2.7 μm, Mobile Phase A: Water/0.02% TFA, Mobile Phase B: Acetonitrile, 5%-95% B-3 min, 1.50 L/min): T_{R}=1.38 min, ES m/z [M+H]⁺: 592, 594.1H NMR (300 MHz, DMSO-d₆) δ 9.31 (s, 1H), 8.61 (s, 1H), 7.52-7.10 (m, 6H), 4.95-4.86 (m, 2H), 4.66 (d, J=4.5 Hz, 1H), 4.28-4.07 (m, 3H), 3.82-3.76 (m, 1H), 3.24-3.05 (m, 2H), 3.01-2.83 (m, 2H), 2.36-2.23 (m, 2H), 2.13-2.07 (m, 1H), 1.87-1.81 (m, 1H), 1.79-1.65 (m, 2H), 1.61-1.55 (m, 1H), 1.41-1.27 (m, 3H).

### Synthesis of (1R,2S,3R,5R)-3-[4-amino-5-(1-benzylpyrazol-3-yl)-2-chloropyrrolo[2,3-d]pyrimidin-7-yl]-5-[1-(cyclopentylmethyl)piperidin-4-yl]cyclopentane-1,2-diol (500)

### (1R,2S,3R,5R)-3-[4-amino-5-(1-benzylpyrazol-3-yl)-2-chloropyrrolo[2,3-d]pyrimidin-7-yl]-5-(piperidin-4-yl)cyclopentane-1,2-diol hydrochloride (501)

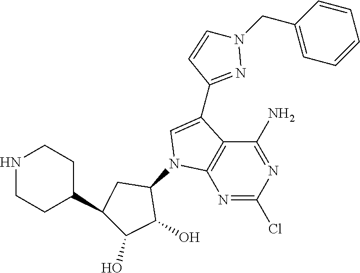 [SMILES: Nc1nc(Cl)nc2c1c(-c1ccn(Cc3ccccc3)n1)cn2[C@@H]1C[C@H](C2CCNCC2)[C@@H](O)[C@H]1O]

Into a 8 mL vial were added tert-butyl 4-[(3aR,4R,6R,6aS)-6-[4-amino-5-(1-benzylpyrazol-3-yl)-2-chloropyrrolo[2,3-d]pyrimidin-7-yl]-2,2-dimethyl-tetrahydro-3aH-cyclopenta[d][1,3]dioxol-4-yl]piperidine-1-carboxylate (0.1 g, 0.15 mmol, 1.0 eq) and 4M HCl in MeOH (1.0 mL) at room temperature. The resulting mixture was stirred for 0.5 h at room temperature. The resulting mixture was concentrated under reduced pressure to afford (1R,2S,3R,5R)-3-[4-amino-5-(1-benzylpyrazol-3-yl)-2-chloropyrrolo[2,3-d]pyrimidin-7-yl]-5-(piperidin-4-yl)cyclopentane-1,2-diol hydrochloride 501 (60 mg, 77%) as a yellow solid. LCMS (conditions Xbridge C18, 50*3.0 mm, 3.5 μm, Mobile Phase A: Water/5 mM NH4HCO₃, Mobile Phase B: Acetonitrile, 5%-100% B-3 min (+), 1.50 L/min, ES, m/z,): T_{R}=1.41 min; [M+H]⁺: 508, 510.

### (1R,2S,3R,5R)-3-[4-amino-5-(1-benzylpyrazol-3-yl)-2-chloropyrrolo[2,3-d]pyrimidin-7-yl]-5-[1-(cyclopentylmethyl)piperidin-4-yl]cyclopentane-1,2-diol (500)

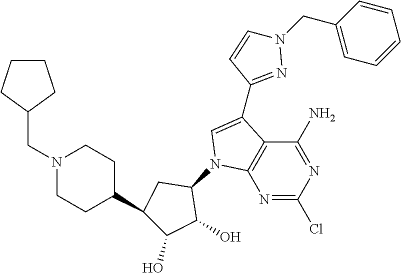 [SMILES: Nc1nc(Cl)nc2c1c(-c1ccn(Cc3ccccc3)n1)cn2[C@@H]1C[C@H](C2CCN(CC3CCCC3)CC2)[C@@H](O)[C@H]1O]

Into a 8 mL vial were added (1R,2S,3R,5R)-3-[4-amino-5-(1-benzylpyrazol-3-yl)-2-chloropyrrolo[2,3-d]pyrimidin-7-yl]-5-(piperidin-4-yl)cyclopentane-1,2-diol 501 (60 mg, 0.1 mmol, 1.0 eq), DIEA (30.5 mg, 0.2 mmol, 2.0 eq) and cyclopentanecarboxaldehyde (23.2 mg, 0.2 mmol, 2.0 eq), NaBH (Oac) 3 (50.1 mg, 0.2 mmol, 2.0 eq) in MeOH (1.0 mL) at room temperature. The resulting mixture was stirred for 1 h at room temperature. The resulting mixture was concentrated under reduced pressure. The residue was dissolved in MeOH (3.0 mL). The residue was purified by Prep-HPLC [column, Xbridge Prep C18 OBD column, 5 μm, 19*150 mm; mobile phase, Water (0.03% NH₄OH) and CH₃CN (15% CH₃CN up to 50% in 10 min); Detector, UV 220&254 nm]to afford (1R,2S,3R,5R)-3-[4-amino-5-(1-benzylpyrazol-3-yl)-2-chloropyrrolo[2,3-d]pyrimidin-7-yl]-5-[1-(cyclopentylmethyl)piperidin-4-yl]cyclopentane-1,2-diol 500 (14.1 mg, 20%) as a white solid. LCMS (conditions: ZORBAX SB-Aq, 50*4.6 mm, 1.8 μm, Mobile Phase A: Water/0.02% TFA, Mobile Phase B: Acetonitrile, 30%-95% B-3 min-1.50 mL/min, ES, m/z): T_{R}=1.22 min; [M+H]⁺: 590, 592. 1H NMR (400 MHZ, DMSO-d₆+D₂O) δ 7.87 (d, J=2.4 Hz, 1H), 7.80 (s, 1H), 7.40-7.29 (m, 2H), 7.33-7.23 (m, 3H), 6.67 (d, J=2.4 Hz, 1H), 5.33 (s, 2H), 4.77-4.66 (m, 1H), 4.07 (dd, J=9.2, 6.2 Hz, 1H), 3.75 (dd, J=6.3, 3.6 Hz, 1H), 2.86 (t, J=10.6 Hz, 2H), 2.17-2.11 (m, 2H), 2.08-1.96 (m, 2H), 1.84-1.77 (m, 3H), 1.69-1.61 (m, 3H), 1.59-1.42 (m, 6H), 1.26-1.08 (m, 5H).

### Synthesis of (1R,2S,3R,5R)-3-(4-amino-5-(1-benzyl-1H-pyrazol-3-yl)-2-chloro-7H-pyrrolo[2,3-d]pyrimidin-7-yl)-5-(piperidin-4-yl)cyclopentane-1,2-diol (501)

### tert-Butyl 4-((3aR,4R,6R,6aS)-6-(4-amino-5-bromo-2-chloro-7H-pyrrolo[2,3-d]pyrimidin-7-yl)-2,2-dimethyltetrahydro-4H-cyclopenta[d][1,3]dioxol-4-yl)piperidine-1-carboxylate (100b)

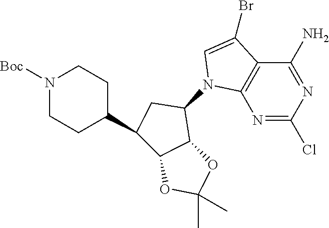 [SMILES: CC(C)(C)OC(=O)N1CCC([C@H]2C[C@@H](n3cc(Br)c4c(N)nc(Cl)nc43)[C@@H]3OC(C)(C)O[C@@H]32)CC1]

Into a 40 mL vial were added tert-butyl 4-[(3aR,4R,6R,6aS)-6-{5-bromo-2,4-dichloropyrrolo[2,3-d]pyrimidin-7-yl}-2,2-dimethyl-tetrahydro-3aH-cyclopenta[d][1,3]dioxol-4-yl]piperidine-1-carboxylate 70b (80 mg, 0.14 mmol, 1.0 eq) and 7M NH3 in MeOH (2.0 mL). The resulting mixture was stirred for 16 h at 70° C., then concentrated under reduced pressure. The residue was purified by silica gel column chromatography (50:50 AcOEt: petroleum ether) to afford tert-butyl 4-[(3aR,4R,6R,6aS)-6-{4-amino-5-bromo-2-chloropyrrolo[2,3-d]pyrimidin-7-yl}-2,2-dimethyl-tetrahydro-3aH-cyclopenta[d][1,3]dioxol-4-yl]piperidine-1-carboxylate 100b (58 mg, 75%) as a yellow solid. LCMS [L-column3 ODS, 50*3.0 mm, 3.0 μm; Mobile Phase A: Water/5 mM NH4HCO₃+0.05% Ammonia, Mobile Phase B: Acetonitrile/5% water, 5%-100% B-3 min, 1.20 mL/min]: T_{R}=2.22 min; ES m/z [M+H]⁺: 570, 572.

### tert-Butyl 4-((3aR,4R,6R,6aS)-6-(4-amino-5-(1-benzyl-1H-pyrazol-3-yl)-2-chloro-7H-pyrrolo[2,3-d]pyrimidin-7-yl)-2,2-dimethyltetrahydro-4H-cyclopenta[d][1,3]dioxol-4-yl)piperidine-1-carboxylate (101b)

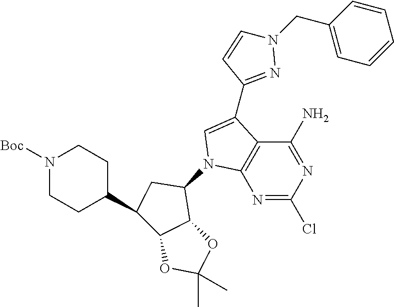 [SMILES: CC(C)(C)OC(=O)N1CCC([C@H]2C[C@@H](n3cc(-c4ccn(Cc5ccccc5)n4)c4c(N)nc(Cl)nc43)[C@@H]3OC(C)(C)O[C@@H]32)CC1]

To a solution of tert-butyl 4-[(3aR,4R,6R,6aS)-6-{4-amino-5-bromo-2-chloropyrrolo[2,3-d]pyrimidin-7-yl}-2,2-dimethyl-tetrahydro-3aH-cyclopenta[d][1,3]dioxol-4-yl]piperidine-1-carboxylate 100b (56 mg, 0.10 mmol, 1.0 eq) and 1-benzylpyrazol-3-ylboronic acid (29 mg, 0.15 mmol, 1.5 eq) [WO201704045]]in dioxane (2.0 mL) and H₂O (0.2 mL) were added K₂CO3 (40 mg, 0.29 mmol, 3.0 eq) and Pd (DtBPF) Cl₂ (12 mg, 0.02 mmol, 0.2 eq). After stirring for 2 h at 90° C. under a nitrogen atmosphere, the reaction was quenched with water (10 mL) and then extracted with EtOAc (3×10 mL). The organic layer was dried over anhydrous Na₂SO₄, filtered and the filtrate was concentrated under reduced pressure. The residue was purified by silica gel column chromatography (50:50 AcOEt: petroleum ether) to afford tert-butyl 4-[(3aR,4R,6R,6aS)-6-[4-amino-5-(1-benzylpyrazol-3-yl)-2-chloropyrrolo[2,3-d]pyrimidin-7-yl]-2,2-dimethyl-tetrahydro-3aH-cyclopenta[d][1,3]dioxol-4-yl]piperidine-1-carboxylate 101b (19 mg, 29%) as a yellow solid. LCMS [Halo C18, 30*3.0 mm, 2.0 μm; Mobile Phase A: Water/0.05% TFA, Mobile Phase B: Acetonitrile, 5%-100% B-2 min, 1.50 mL/min]: T_{R}=1.39 min; ES m/z [M+H]⁺: 648, 650.

### (1R,2S,3R,5R)-3-(4-Amino-5-(1-benzyl-1H-pyrazol-3-yl)-2-chloro-7H-pyrrolo[2,3-d]pyrimidin-7-yl)-5-(piperidin-4-yl)cyclopentane-1,2-diol (501)

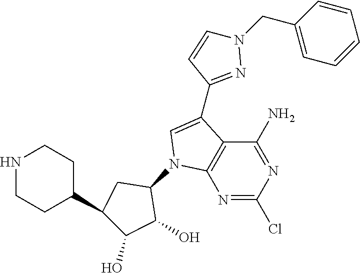 [SMILES: Nc1nc(Cl)nc2c1c(-c1ccn(Cc3ccccc3)n1)cn2[C@@H]1C[C@H](C2CCNCC2)[C@@H](O)[C@H]1O]

Into an 8 mL vial were added tert-butyl 4-[(3aR,4R,6R,6aS)-6-[4-amino-5-(1-benzylpyrazol-3-yl)-2-chloropyrrolo[2,3-d]pyrimidin-7-yl]-2,2-dimethyl-tetrahydro-3aH-cyclopenta[d][1,3]dioxol-4-yl]piperidine-1-carboxylate 101b (19 mg, 0.03 mmol, 1.0 eq) and 4M HCl in MeOH (2.0 mL) at room temperature. The resulting mixture was stirred for 1 h at 50° C. The resulting mixture was concentrated under reduced pressure. The mixture was basified to pH>8 with saturated Na₂CO3 (aq.). The crude product was purified by Prep-HPLC [Column: YMC-Actus Triart C18 ExRS, 30*150 mm, 5 μm; Mobile Phase A: Water (10 mmol/L NH4HCO₃+0.1% NH3·H₂O), Mobile Phase B: ACN; Flow rate: 30 ml/min; Gradient: 20% B to 55% B over 10 min; Detector, UV 220&254 nm]to afford (1R,2S,3R,5R)-3-[4-amino-5-(1-benzylpyrazol-3-yl)-2-chloropyrrolo[2,3-d]pyrimidin-7-yl]-5-(piperidin-4-yl)cyclopentane-1,2-diol 501 (5.2 mg, 34%) as an off-white solid. ¹H NMR (400 MHz, DMSO-d₆+H₂O) δ 7.88 (d, J=2.3 Hz, 1H), 7.80 (s, 1H), 7.38-7.32 (m, 2H), 7.32-7.23 (m, 3H), 6.67 (d, J=2.3 Hz, 1H), 5.33 (s, 2H), 4.72 (q, J=9.2 Hz, 1H), 4.07 (dd, J=9.2, 6.2 Hz, 1H), 3.79-3.72 (m, 1H), 2.95-2.90 (m, 2H), 2.45-2.39 (m, 2H), 2.06-1.95 (m, 1H), 1.82-1.74 (m, 1H), 1.69-1.62 (m, 1H), 1.57-1.42 (m, 2H), 1.40-1.33 (m, 1H), 1.12-1.08 (m, 2H). LCMS [ZORBAX SB-Aq, 50*4.6 mm, 1.8 μm; Mobile Phase A: Water/0.02% TFA, Mobile Phase B: Acetonitrile, 5%-60%-95% B-5 min, 1.50 mL/min]: T_{R}=2.55 min; ES m/z [M+H]⁺: 508, 510. Chiral-HPLC [XA-RP-YMC Cellulose-SC, 4.6*100 mm, 3 μm, Solvent A: Water (0.05% TFA), Solvent B: ACN, Oven Temperature: 25° C., Total Flow: 1.00 mL/min. 5% B-8 min]: T_{R}=4.59 min (8 min run), single peak.

### Synthesis of (1R,2S,3R,5R)-3-(4-amino-5-(1-benzyl-1H-pyrazol-3-yl)-2-chloro-7H-pyrrolo[2,3-d]pyrimidin-7-yl)-5-(1-methylpiperidin-4-yl)cyclopentane-1,2-diol (502)

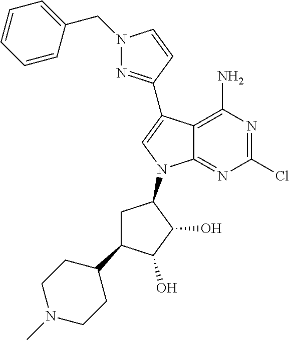 [SMILES: CN1CCC([C@H]2C[C@@H](n3cc(-c4ccn(Cc5ccccc5)n4)c4c(N)nc(Cl)nc43)[C@H](O)[C@@H]2O)CC1]

Into an 8 mL vial were added (1R,2S,3R,5R)-3-[4-amino-5-(1-benzylpyrazol-3-yl)-2-chloropyrrolo[2,3-d]pyrimidin-7-yl]-5-(piperidin-4-yl)cyclopentane-1,2-diol (19 mg, 0.04 mmol, 1.0 eq), DCM (1.0 mL) and HCHO (12 mg in H₂O, 0.37 mmol, 10.0 eq). The resulting mixture was stirred for 2 h at room temperature. CH₃OH (0.5 mL) was added, followed by NaBH₄ (3 mg, 0.07 mmol, 2.0 eq) at 0° C. The resulting mixture was stirred for 1 h at room temperature. The reaction was quenched with water (1.0 mL) at 0° C. The mixture was concentrated under reduced pressure. The residue was purified by Prep-HPLC [Column: YMC-Actus Triart C18 ExRS, 30*150 mm, 5 μm; Mobile Phase A: Water (10 mmol/L NH4HCO₃+0.1% NH3·H₂O), Mobile Phase B: ACN; Flow rate: 30 mL/min; Gradient: 20% B to 48% B in 7 min; Detector, UV 220&254 nm]to afford (1R,2S,3R,5R)-3-[4-amino-5-(1-benzylpyrazol-3-yl)-2-chloropyrrolo[2,3-d]pyrimidin-7-yl]-5-(1-methylpiperidin-4-yl)cyclopentane-1,2-diol 502 (11.2 mg, 57%) as an off-white solid. ¹H NMR (400 MHZ, DMSO-d₆) δ 9.20 (br, s, 1H), 7.90 (s, 1H), 7.84 (s, 1H), 7.59 (br, s, 1H), 7.41-7.22 (m, 5H), 6.68 (d, J=2.3 Hz, 1H), 5.36 (s, 2H), 4.79 (d, J=6.8 Hz, 1H), 4.77-4.69 (m, 1H), 4.58 (d, J=4.8 Hz, 1H), 4.13-4.03 (m, 1H), 3.80-3.72 (m, 1H), 2.76 (t, J=10.8 Hz, 2H), 2.13 (s, 3H), 2.08-1.97 (m, 1H), 1.84-1.77 (m, 3H), 1.69-1.64 (m, 1H), 1.60-1.43 (m, 2H), 1.25-1.20 (m, 3H). LCMS [Cortecs C18+, 50*3.0 mm, 2.7 μm; Mobile Phase A: Water/0.02% TFA, Mobile Phase B: Acetonitrile, 5%-95% B-3 min, 1.50 mL/min]: T_{R}=0.96 min; ES m/z [M+H]⁺: 522, 524. Chiral-HPLC [XA-RP-Lux Cellulose-4, 4.6*150 mm, 3 μm, Solvent A: Water (20 mM NH4HCO₃), Solvent B: Acetonitrile, Oven Temperature: 25° C., Total Flow: 1.00 mL/min, Conc. of Pump B: 40.0%, Run 10 min.]: T_{R}=4.83 min, single peak.

### Synthesis of (1R,2S,3R,5R)-3-(4-amino-5-(4-benzylthiazol-2-yl)-7H-pyrrolo[2,3-d]pyrimidin-7-yl)-5-(1,2,5,6-tetrahydropyridin-3-yl)cyclopentane-1,2-diol (503)

### tert-Butyl 5-((3aR,4R,6aR)-2,2-dimethyl-6-oxotetrahydro-4H-cyclopenta[d][1,3]dioxol-4-yl)-3,6-dihydropyridine-1 (2H)-carboxylate (102b)

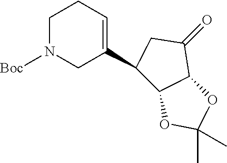 [SMILES: CC(C)(C)OC(=O)N1CCC=C([C@H]2CC(=O)[C@@H]3OC(C)(C)O[C@H]23)C1]

Into a 100 mL sealed tube were added (3aR,6aR)-2,2-dimethyl-3aH,6aH-cyclopenta[d][1,3]dioxol-4-one (6.00 g, 38.91 mmol, 1.0 eq), tert-butyl 3-(4,4,5,5-tetramethyl-1,3,2-dioxaborolan-2-yl)-5,6-dihydro-2H-pyridine-1-carboxylate (18.05 g, 58.38 mmol, 1.5 eq), [Rh(COD) C_{1]2} (3.84 g, 7.78 mmol, 0.2 eq), KOH (0.66 g, 11.67 mmol, 0.3 eq), H₂O (6.0 mL) and CH₃OH (60.0 mL) at room temperature. The reaction mixture was irradiated with microwave radiation for 2 h at 40° C. The reaction was quenched with water (100 mL) and then extracted with EtOAc (3×50 mL). The organic layer was dried over anhydrous Na₂SO₄, filtered and the filtrate was concentrated under reduced pressure. The residue was purified by silica gel column chromatography (5:95 AcOEt: petroleum ether) to afford tert-butyl 3-[(3aR,4R,6aR)-2,2-dimethyl-6-oxo-tetrahydrocyclopenta[d][1,3]dioxol-4-yl]-5,6-dihydro-2H-pyridine-1-carboxylate 102b (3.10 g, 23%) as an off-white solid. LCMS [XBridge C18, 50*3.0 mm, 3.5 μm; Mobile Phase A: Water/5 mM NH4HCO₃, Mobile Phase B: Methanol, 10%-95% B-3 min, 1.00 mL/min]: T_{R}=1.86 min; ES m/z [M+H]⁺: 338.

### tert-Butyl 5-((3aR,4R,6S,6aS)-6-hydroxy-2,2-dimethyltetrahydro-4H-cyclopenta[d][1,3]dioxol-4-yl)-3,6-dihydropyridine-1 (2H)-carboxylate (103b)

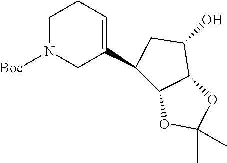 [SMILES: CC(C)(C)OC(=O)N1CCC=C([C@H]2C[C@H](O)[C@@H]3OC(C)(C)O[C@@H]32)C1]

To a stirred solution of tert-butyl 3-[(3aR,4R,6aR)-2,2-dimethyl-6-oxo-tetrahydrocyclopenta[d][1,3]dioxol-4-yl]-5,6-dihydro-2H-pyridine-1-carboxylate 102b (2.30 g, 6.81 mmol, 1.0 eq) in MeOH (25.0 mL) was added NaBH₄ (0.39 g, 10.23 mmol, 1.5 eq) in portions at 0° C. The resulting mixture was stirred for 1 h at room temperature. The reaction was quenched with water (30 mL) and then extracted with EtOAc (3×30 mL). The organic layer was dried over anhydrous Na₂SO₄, filtered and the filtrate was concentrated under reduced pressure. The crude product was purified by Prep-HPLC [Column: Xtimate C18 50×250 mm, 10 μm; Mobile Phase A: 0.5% NH3·H₂O, Mobile Phase B: ACN; Flow rate: 90 mL/min; Gradient: 90% 15 min; Detector, UV 220 nm]to afford tert-butyl 3-[(3aR,4R,6S,6aS)-6-hydroxy-2,2-dimethyl-tetrahydro-3aH-cyclopenta[d][1,3]dioxol-4-yl]-5,6-dihydro-2H-pyridine-1-carboxylate 103b (1.45 g, 62%) as a off-white solid. LCMS [L-column3 ODS, 50*3.0 mm, 3.0 μm; Mobile Phase A: Water/5 mM NH4HCO₃+0.05% Ammonia, Mobile Phase B: Acetonitrile/5% water, 5%-100% B-3 min, 1.00 mL/min]: T_{R}=1.58 min; ES m/z [M+H]⁺: 340.

### tert-Butyl 5-((3aR,4R,6R,6aS)-6-(4-chloro-5-iodo-7H-pyrrolo[2,3-d]pyrimidin-7-yl)-2,2-dimethyltetrahydro-4H-cyclopenta[d][1,3]dioxol-4-yl)-3,6-dihydropyridine-1 (2H)-carboxylate (104b)

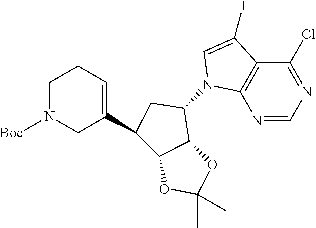 [SMILES: CC(C)(C)OC(=O)N1CCC=C([C@H]2C[C@H](n3cc(I)c4c(Cl)ncnc43)[C@@H]3OC(C)(C)O[C@@H]32)C1]

Into a 40 mL vial were added tert-butyl 3-[(3aR,4R,6S,6aS)-6-hydroxy-2,2-dimethyl-tetrahydro-3aH-cyclopenta[d][1,3]dioxol-4-yl]-5,6-dihydro-2H-pyridine-1-carboxylate 103b (1.45 g, 4.27 mmol, 1.0 eq), 4-chloro-5-iodo-7H-pyrrolo[2,3-d]pyrimidine (1.43 g, 5.12 mmol, 1.2 eq), PPh₃ (2.80 g, 10.68 mmol, 2.5 eq), toluene (20.0 mL) and DBAD (1.97 g, 8.54 mmol, 2.0 eq) at room temperature. The resulting mixture was stirred for 2 h at 50° C. under nitrogen atmosphere. The reaction was quenched with water (30 mL) and then extracted with EtOAc (3×30 mL). The organic layer was dried over anhydrous Na₂SO₄, filtered and the filtrate was concentrated under reduced pressure. The residue was purified by silica gel column chromatography (50:50 AcOEt: petroleum ether) to afford tert-butyl 3-[(3aR,4R,6R,6aS)-6-{4-chloro-5-iodopyrrolo[2,3-d]pyrimidin-7-yl}-2,2-dimethyl-tetrahydro-3aH-cyclopenta[d][1,3]dioxol-4-yl]-5,6-dihydro-2H-pyridine-1-carboxylate 104b (976 mg, 38%) as a yellow solid. LCMS [XBridge C18, 50*3.0 mm, 3.5 μm; Mobile Phase A: Water/5 mM NH4HCO₃, Mobile Phase B: Methanol, 10-95% B-3 min, 1.00 mL/min]: T_{R}=2.36 min [M+H]⁺: 601, 603.

### tert-Butyl 5-((3aR,4R,6R,6aS)-6-(4-amino-5-iodo-7H-pyrrolo[2,3-d]pyrimidin-7-yl)-2,2-dimethyltetrahydro-4H-cyclopenta[d][1,3]dioxol-4-yl)-3,6-dihydropyridine-1 (2H)-carboxylate (105b)

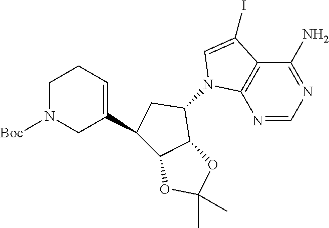 [SMILES: CC(C)(C)OC(=O)N1CCC=C([C@H]2C[C@H](n3cc(I)c4c(N)ncnc43)[C@@H]3OC(C)(C)O[C@@H]32)C1]

Into a 40 mL vial were added tert-butyl 3-[(3aR,4R,6R,6aS)-6-{4-chloro-5-iodopyrrolo[2,3-d]pyrimidin-7-yl}-2,2-dimethyl-tetrahydro-3aH-cyclopenta[d][1,3]dioxol-4-yl]-5,6-dihydro-2H-pyridine-1-carboxylate 104b (370 mg, 0.62 mmol, 1.0 eq) and 7M NH3 in MeOH (5.0 mL). The resulting mixture was stirred for 16 h at 70° C. The resulting mixture was concentrated under reduced pressure. The residue was purified by silica gel column chromatography (50:50 AcOEt: petroleum ether) to afford tert-butyl 3-[(3aR,4R,6R,6aS)-6-{4-amino-5-iodopyrrolo[2,3-d]pyrimidin-7-yl}-2,2-dimethyl-tetrahydro-3aH-cyclopenta[d][1,3]dioxol-4-yl]-5,6-dihydro-2H-pyridine-1-carboxylate 105b (210 mg, 58%) as a yellow solid. LCMS [XBridge C18, 50*3.0 mm, 3.5 μm; Mobile Phase A: Water/5 mM NH₄HCO₃, Mobile Phase B: Methanol, 10-95% B-3 min, 1.00 mL/min]: T_{R}=2.28 min; ES m/z [M+H]⁺: 582.

### tert-Butyl 5-((3aR,4R,6R,6aS)-6-(4-amino-5-(4-benzylthiazol-2-yl)-7H-pyrrolo[2,3-d]pyrimidin-7-yl)-2,2-dimethyltetrahydro-4H-cyclopenta[d][1,3]dioxol-4-yl)-3,6-dihydropyridine-1 (2H)-carboxylate (106b)

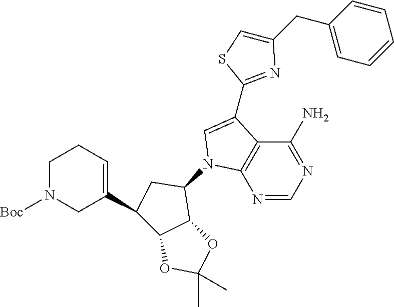 [SMILES: CC(C)(C)OC(=O)N1CCC=C([C@H]2C[C@@H](n3cc(-c4nc(Cc5ccccc5)cs4)c4c(N)ncnc43)[C@@H]3OC(C)(C)O[C@@H]32)C1]

To a solution of tert-butyl 3-[(3aR,4R,6R,6aS)-6-{4-amino-5-iodopyrrolo[2,3-d]pyrimidin-7-yl}-2,2-dimethyl-tetrahydro-3aH-cyclopenta[d][1,3]dioxol-4-yl]-5,6-dihydro-2H-pyridine-1-carboxylate 105b (187 mg, 0.32 mmol, 1.0 eq) and (4-benzyl-1,3-thiazol-2-yl)(chloro) zinc (2.5 mL in THF, 0.64 mmol, 2.0 eq) in toluene (4.0 mL) were added Pd (PPh₃) 2Cl₂ (45 mg, 0.06 mmol, 0.2 eq) and K₃PO₄ (204 mg, 0.97 mmol, 3.0 eq). After stirring for 18 h at 60° C. under a nitrogen atmosphere, the resulting mixture was concentrated under reduced pressure, then extracted with EtOAc (3×10 mL). The organic layer was dried over anhydrous Na₂SO₄, filtered and the filtrate was concentrated under reduced pressure. The residue was purified by silica gel column chromatography (50:50 AcOEt: petroleum ether) to afford tert-butyl 3-[(3aR,4R,6R,6aS)-6-[4-amino-5-(4-benzyl-1,3-thiazol-2-yl)pyrrolo[2,3-d]pyrimidin-7-yl]-2,2-dimethyl-tetrahydro-3aH-cyclopenta[d][1,3]dioxol-4-yl]-5,6-dihydro-2H-pyridine-1-carboxylate 106b (74 mg, 36%) as a yellow solid. LCMS [XBridge C18, 50*3.0 mm, 3.5 μm; Mobile Phase A: Water/5 mM NH₄HCO₃, Mobile Phase B: Methanol, 10-95% B-3 min, 1.00 mL/min]: T_{R}=2.47 min; ES m/z [M+H]⁺: 629.

### (1R,2S,3R,5R)-3-(4-amino-5-(4-benzylthiazol-2-yl)-7H-pyrrolo[2,3-d]pyrimidin-7-yl)-5-(1,2,5,6-tetrahydropyridin-3-yl)cyclopentane-1,2-diol (503)

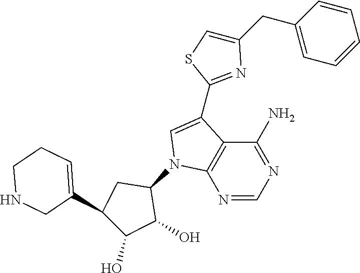 [SMILES: Nc1ncnc2c1c(-c1nc(Cc3ccccc3)cs1)cn2[C@@H]1C[C@H](C2=CCCNC2)[C@@H](O)[C@H]1O]

Into an 8 mL vial were added tert-butyl 3-[(3aR,4R,6R,6aS)-6-[4-amino-5-(4-benzyl-1,3-thiazol-2-yl)pyrrolo[2,3-d]pyrimidin-7-yl]-2,2-dimethyl-tetrahydro-3aH-cyclopenta[d][1,3]dioxol-4-yl]-5,6-dihydro-2H-pyridine-1-carboxylate 106b (30 mg, 0.05 mmol, 1.0 eq) and 4M HCl in MeOH (2.0 mL). The resulting mixture was stirred for 1 h at 50° C. The reaction was concentrated under reduced pressure. The mixture was basified to pH>8 with saturated Na₂CO3 (aq.). The crude product was purified by Prep-HPLC [Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 20% B to 58% B in 9 min; Detector, UV 220&254 nm]to afford (1R,2S,3R,5R)-3-[4-amino-5-(4-benzyl-1,3-thiazol-2-yl)pyrrolo[2,3-d]pyrimidin-7-yl]-5-(1,2,5,6-tetrahydropyridin-3-yl)cyclopentane-1,2-diol 503 (5.2 mg, 22%) as an off-white solid. ¹H NMR (400 MHZ, DMSO-d₆+H₂O) δ 8.11-8.02 (m, 2H), 7.36-7.26 (m, 4H), 7.26-7.14 (m, 2H), 5.61 (s, 1H), 4.80 (dt, J=11.1, 7.5 Hz, 1H), 4.20 (t, J=7.2 Hz, 1H), 4.04 (s, 2H), 3.87 (t, J=6.2 Hz, 1H), 3.33-3.24 (m, 1H), 3.18-3.09 (m, 1H), 2.75-2.70 (m, 2H), 2.39-2.34 (m, 1H), 2.09-1.94 (m, 3H), 1.92-1.79 (m, 1H). LCMS [Cortecs C18+, 50*3.0 mm, 2.7 μm; Mobile Phase A: Water/0.02% TFA, Mobile Phase B: Acetonitrile, 5%-20%-95% B-5 min, 1.50 mL/min]: T_{R}=2.79 min; ES m/z [M+H]⁺: 489.

### Synthesis of (1R,2S,3R,5R)-3-[4-amino-5-(4-benzyl-1,3-thiazol-2-yl)-2-chloropyrrolo[2,3-d]pyrimidin-7-yl]-5-(1-ethylpiperidin-4-yl)cyclopentane-1,2-diol (504)

### tert-butyl 4-[(3aR,4R,6R,6aS)-6-{5-bromo-2,4-dichloropyrrolo[2,3-d]pyrimidin-7-yl}-2,2-dimethyl-tetrahydro-3aH-cyclopenta[d][1,3]dioxol-4-yl]piperidine-1-carboxylate (107b)

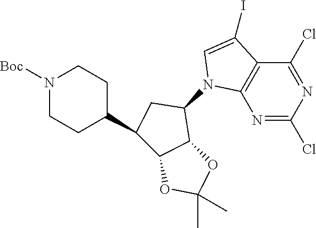 [SMILES: CC(C)(C)OC(=O)N1CCC([C@H]2C[C@@H](n3cc(I)c4c(Cl)nc(Cl)nc43)[C@@H]3OC(C)(C)O[C@@H]32)CC1]

Into a 40 mL vial was placed a solution of tert-butyl 4-[(3aR,4R,6S,6aS)-6-hydroxy-2,2-dimethyl-tetrahydro-3aH-cyclopenta[d][1,3]dioxol-4-yl]piperidine-1-carboxylate 3 (1.54 g, 4.5 mmol, 1.2 eq) in toluene (15.0 mL). 5-Bromo-2,4-dichloro-7H-pyrrolo[2,3-d]pyrimidine (1.00 g, 3.7 mmol, 1.0 eq) and PPh₃ (1.97 g, 7.5 mmol, 2.0 eq) were added, and the mixture was cooled to 0° C. To this was added DBAD (1.73 g, 7.5 mmol, 2.0 eq) in toluene (7.0 mL) dropwise over 5 min at 0° C. The resulting mixture was stirred for 5 h at 50° C., then concentrated under reduced pressure. The residue was dissolved in DCM (2.0 mL) and purified by silica gel column chromatography (78:22 Petroleum ether/AcOEt) to afford tert-butyl 4-[(3aR,4R,6R,6aS)-6-{5-bromo-2,4-dichloropyrrolo[2,3-d]pyrimidin-7-yl}-2,2-dimethyl-tetrahydro-3aH-cyclopenta[d][1,3]dioxol-4-yl]piperidine-1-carboxylate 107b (1.7 g, 76%) as an off-white solid. LCMS (conditions Proshell HPH-C18, 50*3.0 mm, 2.7 μm, Mobile Phase A: Water (0.05% ammonia water), Mobile Phase B: Acetonitrile, 2%-100% B-2 min (+), 1.50 L/min): T_{R}=1.01 min; ES m/z [M+H]⁺: 637,639.

### Tert-butyl 4-[(3aR,4R,6R,6aS)-6-{4-amino-2-chloro-5-iodopyrrolo[2,3-d]pyrimidin-7-yl}-2,2-dimethyl-tetrahydro-3aH-cyclopenta[d][1,3]dioxol-4-yl]piperidine-1-carboxylate (108b)

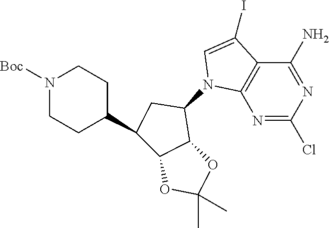 [SMILES: CC(C)(C)OC(=O)N1CCC([C@H]2C[C@@H](n3cc(I)c4c(N)nc(Cl)nc43)[C@@H]3OC(C)(C)O[C@@H]32)CC1]

Into a 40 mL vial was placed a solution of tert-butyl 4-[(3aR,4R,6R,6aS)-6-{2,4-dichloro-5-iodopyrrolo[2,3-d]pyrimidin-7-yl}-2,2-dimethyl-tetrahydro-3aH-cyclopenta[d][1,3]dioxol-4-yl]piperidine-1-carboxylate 107b (1.50 g, 2.3 mmol, 1.0 eq) in 7M NH₃ in MeOH (15.0 mL). The mixture was stirred for 6 h at 80° C. The resulting mixture was concentrated under reduced pressure. The residue was purified by silica gel column chromatography (93:7 DCM: MeOH) to afford tert-butyl 4-[(3aR,4R,6R,6aS)-6-{4-amino-2-chloro-5-iodopyrrolo[2,3-d]pyrimidin-7-yl}-2,2-dimethyl-tetrahydro-3aH-cyclopenta[d][1,3]dioxol-4-yl]piperidine-1-carboxylate 108b (1.2 g, 82%) as an off-white solid. LCMS (conditions: L-column3 ODS, 50*3.0 mm, 3.0 μm, Mobile Phase Water/5 mM NH₄HCO₃+0.05% Ammonia, Mobile Phase B: Acetonitrile/5% water, 2%-100% B-3 min, 1.50 L/min): T_{R}=2.23 min; ES m/z [M+H]⁺: 618, 620.

### Tert-butyl4-[(3aR,4R,6R,6aS)-6-[4-amino-5-(4-benzyl-1,3-thiazol-2-yl)-2-chloropyrrolo[2,3-d]pyrimidin-7-yl]-2,2-dimethyl-tetrahydro-3aH-cyclopenta[d][1,3]dioxol-4-yl]piperidine-1-carboxylate (109b)

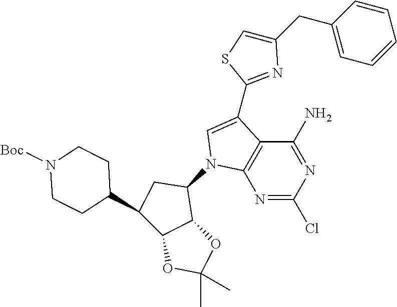 [SMILES: CC(C)(C)OC(=O)N1CCC([C@H]2C[C@@H](n3cc(-c4nc(Cc5ccccc5)cs4)c4c(N)nc(Cl)nc43)[C@@H]3OC(C)(C)O[C@@H]32)CC1]

Into a 40 mL vial was placed a solution of tert-butyl 4-[(3aR,4R,6R,6aS)-6-{4-amino-2-chloro-5-iodopyrrolo[2,3-d]pyrimidin-7-yl}-2,2-dimethyl-tetrahydro-3aH-cyclopenta[d][1,3]dioxol-4-yl]piperidine-1-carboxylate 108b (0.60 g, 1.0 mmol, 1.0 eq) in toluene (6.0 mL). Then Pd (PPh₃) 2Cl₂ (68 mg, 0.1 mmol, 0.1 eq) was added. To the mixture was added (4-benzyl-1,3-thiazol-2-yl)(chloro) zinc (0.93 g, 3.4 mmol, 3.5 eq) dropwise under N₂ atmosphere. The resulting mixture was stirred overnight at 60° C. The reaction was quenched with saturated NH₄Cl solution (10.0 mL) at room temperature. The resulting mixture was extracted with AcOEt (3×10.0 mL). The combined organic layers were washed with brine, dried over anhydrous Na₂SO₄. After filtration, the filtrate was concentrated under reduced pressure. The residue was purified by silica gel column chromatography (70:30 Petroleum ether: THF) to afford tert-butyl 4-[(3aR,4R,6R,6aS)-6-[4-amino-5-(4-benzyl-1,3-thiazol-2-yl)-2-chloropyrrolo[2,3-d]pyrimidin-7-yl]-2,2-dimethyl-tetrahydro-3aH-cyclopenta[d][1,3]dioxol-4-yl]piperidine-1-carboxylate 109b (600 mg, 92%) as light yellow oil. LCMS (conditions: L-column3 ODS, 50*3.0 mm, 3.0 μm, Mobile Phase Water/5 mM NH₄HCO₃+0.05% Ammonia, Mobile Phase B: Acetonitrile/5% water, 2%-100% B-3 min, 1.50 L/min): T_{R}=2.23 min; ES m/z [M+H]⁺: 665, 667.

### (1R,2S,3R,5R)-3-[4-amino-5-(4-benzyl-1,3-thiazol-2-yl)-2-chloropyrrolo[2,3-d]pyrimidin-7-yl]-5-(piperidin-4-yl)cyclopentane-1,2-diol (110b)

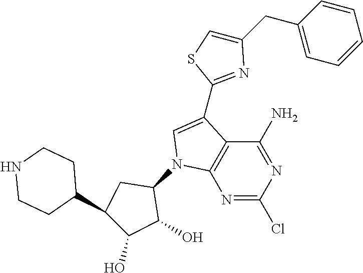 [SMILES: Nc1nc(Cl)nc2c1c(-c1nc(Cc3ccccc3)cs1)cn2[C@@H]1C[C@H](C2CCNCC2)[C@@H](O)[C@H]1O]

Into a 40 mL vial was placed a solution of tert-butyl 4-[(3aR,4R,6R,6aS)-6-[4-amino-5-(4-benzyl-1,3-thiazol-2-yl)-2-chloropyrrolo[2,3-d]pyrimidin-7-yl]-2,2-dimethyl-tetrahydro-3aH-cyclopenta[d][1,3]dioxol-4-yl]piperidine-1-carboxylate 109b (200 mg, 0.3 mmol, 1.0 eq) in 4M HCl in MeOH (4.0 mL). The mixture was stirred for 20 min at 60° C. The resulting mixture was concentrated under reduced pressure to afford crude product (1R,2S,3R,5R)-3-[4-amino-5-(4-benzyl-1,3-thiazol-2-yl)-2-chloropyrrolo[2,3-d]pyrimidin-7-yl]-5-(piperidin-4-yl)cyclopentane-1,2-diol 110b (50 mg) as a light yellow solid. This was used in the next step without purification. LCMS (conditions: L-column3 ODS, 50*3.0 mm, 3.0 μm, Mobile Phase Water/5 mM NH₄HCO₃+0.05% Ammonia, Mobile Phase B: Acetonitrile/5% water, 2%-100% B-3 min, 1.50 L/min): T_{R}=1.57 min; ES m/z [M+H]⁺: 525, 527.

### (1R,2S,3R,5R)-3-[4-amino-5-(4-benzyl-1,3-thiazol-2-yl)-2-chloropyrrolo[2,3-d]pyrimidin-7-yl]-5-(1-ethylpiperidin-4-yl)cyclopentane-1,2-diol (504)

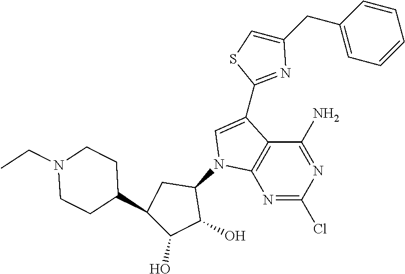 [SMILES: CCN1CCC([C@H]2C[C@@H](n3cc(-c4nc(Cc5ccccc5)cs4)c4c(N)nc(Cl)nc43)[C@H](O)[C@@H]2O)CC1]

Into a 40 mL vial was placed a solution of (1R,2S,3R,5R)-3-[4-amino-5-(4-benzyl-1,3-thiazol-2-yl)-2-chloropyrrolo[2,3-d]pyrimidin-7-yl]-5-(piperidin-4-yl)cyclopentane-1,2-diol 110b (50 mg, 0.1 mmol, 1.0 eq) in DCM (2.0 mL) and DIEA (24 mg, 0.2 mmol, 2.0 eq). NaBH (AcO) 3 (40 mg, 0.2 mmol, 2.0 eq) was added. The mixture was stirred for 10 min, then concentrated under reduced pressure. The residue was dissolved in MeOH (2.0 mL). The mixture was purified by Prep-HPLC (Column: YMC-Actus Triart C18, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 20% B to 45% B in 7 min, 50% B; Wavelength: 220 nm) to afford (1R,2S,3R,5R)-3-[4-amino-5-(4-benzyl-1,3-thiazol-2-yl)-2-chloropyrrolo[2,3-d]pyrimidin-7-yl]-5-(1-ethylpiperidin-4-yl)cyclopentane-1,2-diol 504 (11.9 mg, 22%) as an off-white solid. LCMS (conditions Cortecs C18+, 50*3.0 mm, 2.7 μm, Mobile Phase A: Water/0.02% TFA, Mobile Phase B: Acetonitrile, 2%-100% B-3 min, 1.50 L/min): T_{R}=1.10 min; ES m/z [M+H]⁺: 553, 555. ¹H NMR (400 MHZ, DMSO-d₆+D₂O) 88.09 (s, 1H), 7.36-7.17 (m, 6H), 4.78-4.67 (m, 1H), 4.17 (dd, J=9.0, 6.5 Hz, 1H), 4.05 (s, 2H), 3.79 (dd, J=6.5, 4.0 Hz, 1H), 3.47 (t, J=11.2 Hz, 2H), 3.19-3.00 (m, 2H), 2.82 (q, J=13.8 Hz, 2H), 2.15-2.01 (m, 2H), 1.87-1.79 (m, 1H), 1.75-1.34 (m, 5H), 1.21 (t, J=7.3 Hz, 3H).

### Synthesis of (1R,2S,3R,5R)-3-(4-amino-5-(4-benzylthiazol-2-yl)-2-chloro-7H-pyrrolo[2,3-d]pyrimidin-7-yl)-5-(1-methylpiperidin-4-yl)cyclopentane-1,2-diol (505)

### (1R,2S,3R,5R)-3-(4-amino-5-(4-benzylthiazol-2-yl)-2-chloro-7H-pyrrolo[2,3-d]pyrimidin-7-yl)-5-(1-methylpiperidin-4-yl)cyclopentane-1,2-diol (505)

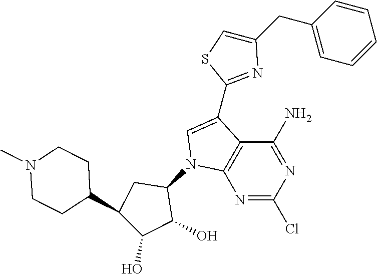 [SMILES: CN1CCC([C@H]2C[C@@H](n3cc(-c4nc(Cc5ccccc5)cs4)c4c(N)nc(Cl)nc43)[C@H](O)[C@@H]2O)CC1]

Into a 40 mL vial were added (1R,2S,3R,5R)-3-[4-amino-5-(4-benzyl-1,3-thiazol-2-yl)-2-chloropyrrolo[2,3-d]pyrimidin-7-yl]-5-(piperidin-4-yl)cyclopentane-1,2-diol 110b (80 mq, 0.15 mmol, 1.0 eq), 30% HCHO in H₂O (31 mg, 0.31 mmol, 2.0 eq), AcOH (0.46 mg, 0.01 mmol, 0.05 eq) and DCM (5.0 ml) at 0° C. To this was added NaBH (OAc) 3 (64.6 mg, 0.30 mmol, 2.0 eq) in portions over 2 min at 0° C. The resulting mixture was stirred for 2 h, then concentrated under reduced pressure. The residue was dissolved in MeOH (2.0 mL). The mixture was purified Prep-HPLC (Column: YMC-Actus Triart C18, 30*150 mm, 5 μm; Mobile Phase A: Water (10 mmol/L NH₄HCO₃+0.1% NH₃·H₂O), Mobile Phase B: ACN; Flow rate: 30 ml/min; Gradient: 20% B to 45% B in 7 min, 50% B; Wavelength: 220 nm) to afford (1R,2S,3R,5R)-3-[4-amino-5-(4-benzyl-1,3-thiazol-2-yl)-2-chloropyrrolo[2,3-d]pyrimidin-7-yl]-5-(1-methylpiperidin-4-yl)cyclopentane-1,2-diol 505 (24.3 mg, 29%) as an off-white solid. LCMS (Cortecs C18+, 50*3.0 mm, 2.7 μm, Mobile Phase A: Water/0.02% TFA, Mobile Phase B: Acetonitrile, 5%-95% B-3 min, 1.50 L/min): T_{R}=1.07 min; ES m/z [M+H]⁺: 539, 541. ¹H NMR (300 MHZ, DMSO-d₆+D₂O) δ 9.44 (s, 1H), 8.12 (s, 1H), 7.76 (s, 1H), 7.38-7.20 (m, 6H), 4.84-4.64 (m, 1H), 4.15 (dd, J=9.3, 6.4 Hz, 1H), 4.05 (s, 2H), 3.83-3.69 (m, 1H), 3.11-2.90 (m, 2H), 2.35 (s, 3H), 2.28-2.10 (m, 2H), 2.08-1.98 (m, 1H), 1.97-1.90 (m, 1H), 1.70-1.51 (m, 3H), 1.39-1.20 (m, 3H).

### Synthesis of (1R,2S,3R,5R)-3-[4-amino-5-(4-benzyl-1,3-thiazol-2-yl)-2-chloropyrrolo[2,3-d]pyrimidin-7-yl]-5-(1-isopropylpiperidin-4-yl)cyclopentane-1,2-diol (506)

### (1R,2S,3R,5R)-3-[4-amino-5-(4-benzyl-1,3-thiazol-2-yl)-2-chloropyrrolo[2,3-d]pyrimidin-7-yl]-5-(1-isopropylpiperidin-4-yl)cyclopentane-1,2-diol (506)

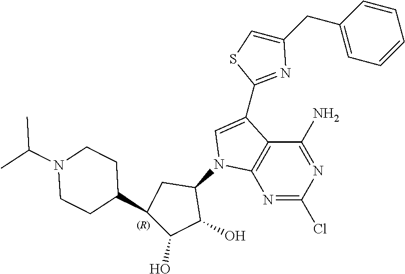

Into a 40 mL vial was placed a solution of (1R,2S,3R,5R)-3-[4-amino-5-(4-benzyl-1,3-thiazol-2-yl)-2-chloropyrrolo[2,3-d]pyrimidin-7-yl]-5-(piperidin-4-yl)cyclopentane-1,2-diol 110b (50.0 mg, 0.1 mmol, 1.0 eq) in DCM (2.0 mL). Then acetone (11.1 mg, 0.2 mmol, 2.0 eq), DIEA (24.6 mg, 0.2 mmol, 2.0 eq) and NaBH (OAc) 3 (40 mg, 0.2 mmol, 2.0 eq) were added. The mixture was stirred for, then concentrated under reduced pressure. The residue was dissolved in MeOH (2.0 mL). The mixture was purified by Prep-HPLC (Column: Welch Ultimate XB-C18, 50*250 mm, 10 μm; Mobile Phase A: 0.1% HCl, Mobile Phase B: ACN; Flow rate: 90 mL/min; Gradient: 5% B-5% B-3 min, 10% B-50% B-12 mim) to afford (1R,2S,3R,5R)-3-[4-amino-5-(4-benzyl-1,3-thiazol-2-yl)-2-chloropyrrolo[2,3-d]pyrimidin-7-yl]-5-(1-isopropylpiperidin-4-yl)cyclopentane-1,2-diol 2HCl salt 506 (13.9 mg, 25%) as an off-white solid. LCMS (ZORBAX SB-Aq, 50*4.6 mm, 1.8 μm, Mobile Phase A: Water/0.02% TFA, Mobile Phase B: Acetonitrile, 5%-(25%-55%)-95% B-5 min, 1.50 L/min): T_{R}=2.55 min; ES m/z [M+H]⁺: 567, 569.1H NMR (400 MHZ, DMSO-d₆+D₂O) 88.09 (s, 1H), 7.40-7.11 (m, 6H), 4.78-4.67 (m, 1H), 4.17 (dd, J=9.1, 6.4 Hz, 1H), 4.05 (s, 2H), 3.83-3.77 (m, 1H), 3.49-3.27 (m, 3H), 2.96-2.81 (m, 2H), 2.16-2.02 (m, 2H), 1.88-1.81 (m, 1H), 1.75-1.61 (m, 1H), 1.63-1.50 (m, 2H), 1.50-1.43 (m, 2H), 1.24 (d, J=6.6 Hz, 6H).

### Synthesis of (1R,2S,3R,5R)-3-[4-amino-5-(4-benzyl-1,3-thiazol-2-yl)-2-chloropyrrolo[2,3-d]pyrimidin-7-yl]-5-[1-(cyclopropylmethyl)piperidin-4-yl]cyclopentane-1,2-diol (507)

### (1R,2S,3R,5R)-3-[4-amino-5-(4-benzyl-1,3-thiazol-2-yl)-2-chloropyrrolo[2,3-d]pyrimidin-7-yl]-5-[1-(cyclopropylmethyl)piperidin-4-yl]cyclopentane-17-diol (507)

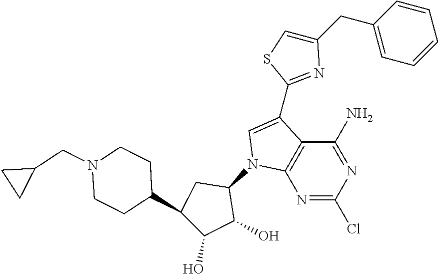 [SMILES: Nc1nc(Cl)nc2c1c(-c1nc(Cc3ccccc3)cs1)cn2[C@@H]1C[C@H](C2CCN(CC3CC3)CC2)[C@@H](O)[C@H]1O]

Into a 40 mL vial was placed a solution of (1R,2S,3R,5R)-3-[4-amino-5-(4-benzyl-1,3-thiazol-2-yl)-2-chloropyrrolo[2,3-d]pyrimidin-7-yl]-5-(piperidin-4-yl)cyclopentane-1,2-diol 110b (50 mg, 0.1 mmol, 1.0 eq) in DCM (2.0 mL). Then cyclopropanecarbaldehyde (13.35 mg, 0.2 mmol, 2.0 eq), DIEA (24.62 mg, 0.2 mmol, 2.0 eq) and NaBH (OAc) 3 (40 mg, 0.2 mmol, 2.0 eq) were added. The mixture was stirred for 10 min, then concentrated under reduced pressure. The residue was dissolved in MeOH (2.0 mL). The mixture was purified by Prep-HPLC (Column: Welch Ultimate XB-C18, 50*250 mm, 10 μm; Mobile Phase A: 0.1% HCl, Mobile Phase B: ACN; Flow rate: 90 mL/min; Gradient: 5% B-5% B-3 min, 10% B-50% B-12 mim) to afford (1R,2S,3R,5R)-3-[4-amino-5-(4-benzyl-1,3-thiazol-2-yl)-2-chloropyrrolo[2,3-d]pyrimidin-7-yl]-5-[1-(cyclopropylmethyl)piperidin-4-yl]cyclopentane-1,2-diol 2HCl 507 (17.3 mg, 31%) as an off-white solid. LCMS (Cortecs C18+, 50*3.0 mm, 2.7 μm, Mobile Phase A: Water/0.02% TFA, Mobile Phase B: Acetonitrile, 5%-95% B-3 min, 1.50 L/min): T_{R}=1.16 min; ES m/z [M+H]⁺: 579, 581 ¹H NMR (400 MHZ, DMSO-d₆+D₂O) 88.10 (s, 1H), 7.36-7.17 (m, 6H), 4.79-4.67 (m, 1H), 4.17 (dd, J=9.1, 6.4 Hz, 1H), 4.05 (s, 2H), 3.80 (dd, J=6.6, 4.1 Hz, 1H), 3.61-3.49 (m, 2H), 2.94 (d, J=7.4 Hz, 2H), 2.91-2.80 (m, 2H), 2.11-2.06 (m, 2H), 1.93-1.78 (m, 1H), 1.72-1.67 (m, 1H), 1.65-1.35 (m, 4H), 1.16-0.93 (m, 1H), 0.68-0.59 (m, 2H), 0.40-0.31 (m, 2H).

### Synthesis of (1R,2S,3R)-3-[4-amino-5-(4-benzyl-1,3-thiazol-2-yl)-2-chloropyrrolo[2,3-d]pyrimidin-7-yl]-5-[1-(2-fluoroethyl)piperidin-4-yl]cyclopentane-1,2-diol (508)

### (1R,2S,3R)-3-[4-Amino-5-(4-benzyl-1,3-thiazol-2-yl)-2-chloropyrrolo[2,3-d]pyrimidin-7-yl]-5-[1-(2-fluoroethyl)piperidin-4-yl]cyclopentane-1,2-diol (508)

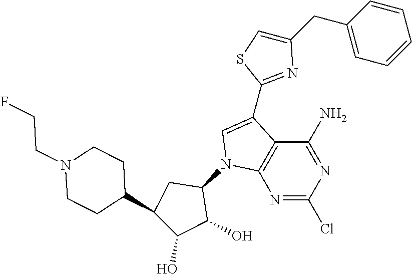 [SMILES: Nc1nc(Cl)nc2c1c(-c1nc(Cc3ccccc3)cs1)cn2[C@@H]1C[C@H](C2CCN(CCF)CC2)[C@@H](O)[C@H]1O]

Into an 8 mL vial was placed a solution of (1R,2S,3R)-3-[4-amino-5-(4-benzyl-1,3-thiazol-2-yl)-2-chloropyrrolo[2,3-d]pyrimidin-7-yl]-5-(piperidin-4-yl)cyclopentane-1,2-diol 110b (50.0 mg, 0.1 mmol, 1.0 eq) in dioxane (1.0 mL). 2-Fluoroethyl methanesulfonate (27.1 mg, 0.2 mmol, 2.0 eq) [WO2018148367]and Na₂CO₃ (20.2 mg, 0.2 mmol, 2.0 eq) were added. The mixture was stirred for 16 h at 80° C., then concentrated. The residue was purified by Prep-HPLC (Column: Welch Ultimate XB-C18, 50*250 mm, 10 μm; Mobile Phase A: 0.1% HCl, Mobile Phase B: ACN; Flow rate: 90 mL/min; Gradient: 5% B-5% B-3 min, 10% B-45% B-12 mim) to afford (1R,2S,3R)-3-[4-amino-5-(4-benzyl-1,3-thiazol-2-yl)-2-chloropyrrolo[2,3-d]pyrimidin-7-yl]-5-[1-(2-fluoroethyl)piperidin-4-yl]cyclopentane-1,2-diol dihydrochloride salt 508 (14.2 mg, 26%) as an off-white solid. LCMS (Cortecs C18+, 50*3.0 mm, 2.7 μm, Mobile Phase A: Water/0.02% TFA, Mobile Phase B: Acetonitrile, 5%-(25%-55%)-95% B-5 min, 1.50 L/min): T_{R}=1.32 min; ES m/z [M+H]⁺: 571,573. 1H NMR (400 MHZ, DMSO-d₆+D₂O) δ 8.10 (s, 1H), 7.46-7.02 (m, 6H), 4.92-4.85 (m, 1H), 4.80-4.67 (m, 2H), 4.17 (dd, J=9.1, 6.4 Hz, 1H), 4.05 (s, 2H), 3.80 (dd, J=6.5, 3.9 Hz, 1H), 3.58-3.45 (m, 3H), 3.44-3.37 (m, 1H), 2.97 (q, J=13.3 Hz, 2H), 2.15-2.01 (m, 2H), 1.87-1.79 (m, 1H), 1.74-1.42 (m, 5H).

### Synthesis of (1R,2S,3R,5R)-3-[4-amino-5-(4-benzyl-1,3-thiazol-2-yl)-2-chloropyrrolo[2,3-d]pyrimidin-7-yl]-5-[1-(prop-2-en-1-yl)piperidin-4-yl]cyclopentane-1,2-diol (509)

### (1R,2S,3R,5R)-3-[4-amino-5-(4-benzyl-1,3-thiazol-2-yl)-2-chloropyrrolo[2,3-d]pyrimidin-7-yl]-5-[1-(prop-2-en-1-yl)piperidin-4-yl]cyclopentane-1,2-diol (509)

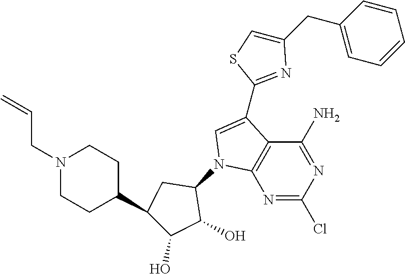 [SMILES: C=CCN1CCC([C@H]2C[C@@H](n3cc(-c4nc(Cc5ccccc5)cs4)c4c(N)nc(Cl)nc43)[C@H](O)[C@@H]2O)CC1]

Into a 8 vial was placed a solution of (1R,2S,3R)-3-[4-amino-5-(4-benzyl-1,3-thiazol-2-yl)-2-chloropyrrolo[2,3-d]pyrimidin-7-yl]-5-(piperidin-4-yl)cyclopentane-1,2-diol 110b (50.0 mg, 0.1 mmol, 1.0 eq) in dioxane (1.0 mL). Bromocyclopropane (69.1 mg, 0.6 mmol, 6.0 eq) and Na₂CO₃ (20.2 mg, 0.2 mmol, 2.0 eq) were added. The mixture was stirred for 16 h at 80° C. The resulting mixture was filtered, the filter cake being washed with MeOH (1.0 mL). The filtrate was purified by Prep-HPLC (Column: Welch Ultimate XB-C18, 50*250 mm, 10 μm; Mobile Phase A: 0.1% HCl, Mobile Phase B: ACN; Flow rate: 90 mL/min; Gradient: 5% B-5% B-3 min, 10% B-50% B-12 mim) to afford (1R,2S,3R,5R)-3-[4-amino-5-(4-benzyl-1,3-thiazol-2-yl)-2-chloropyrrolo[2,3-d]pyrimidin-7-yl]-5-[1-(prop-2-en-1-yl)piperidin-4-yl]cyclopentane-1,2-diol 2HCl salt 509 (6.9 mg, 12%) as an off-white solid. LCMS (Cortecs C18+, 50*3.0 mm, 2.7 μm, Mobile Phase A: Water/0.02% TFA, Mobile Phase B: Acetonitrile, 5%-95% B-3 min, 1.50 L/min): T_{R}=1.10 min; ES m/z [M+H]⁺: 565, 567. ¹H NMR (400 MHZ, DMSO-d₆) δ8.10 (s, 1H), 7.40-7.18 (m, 6H), 5.99-5.83 (m, 1H), 5.57-5.49 (m, 2H), 4.74 (q, J=9.2 Hz, 1H), 4.18 (dd, J=9.0, 6.5 Hz, 1H), 4.06 (s, 2H), 3.85-3.75 (m, 1H), 3.74-3.70 (m, 2H), 3.53-3.38 (m, 2H), 2.94-2.76 (m, 2H), 2.18-2.02 (m, 2H), 1.90-1.80 (m, 1H), 1.74-1.36 (m, 5H).

### Synthesis of 1-{4-[(1R,2R,3S,4R)-4-[4-amino-5-(4-benzyl-1,3-thiazol-2-yl)-2-chloropyrrolo[2,3-d]pyrimidin-7-yl]-2,3-dihydroxycyclopentyl]piperidin-1-yl}propan-2-one (510)

### 1-{4-[(1R,2R,3S,4R)-4-[4-amino-5-(4-benzyl-1,3-thiazol-2-yl)-2-chloropyrrolo[2,3-d]pyrimidin-7-yl]-2,3-dihydroxycyclopentyl]piperidin-1-yl]propan-?-one (510)

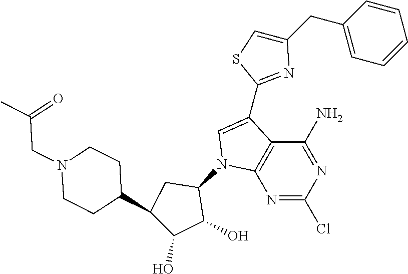 [SMILES: CC(=O)CN1CCC([C@H]2C[C@@H](n3cc(-c4nc(Cc5ccccc5)cs4)c4c(N)nc(Cl)nc43)[C@H](O)[C@@H]2O)CC1]

Into a 40 mL vial was placed a solution of (1R,2S,3R,5R)-3-[4-amino-5-(4-benzyl-1,3-thiazol-2-yl)-2-chloropyrrolo[2,3-d]pyrimidin-7-yl]-5-(piperidin-4-yl)cyclopentane-1,2-diol 110b (50.0 mg, 0.1 mmol, 1.0 eq) in DMF (1.0 mL). TEA (19.3 mg, 0.2 mmol, 2.0 eq) and bromoacetone (19.6 mg, 0.1 mmol, 1.5 eq) were added. The mixture was stirred for 1 h, then filtered, the filter cake being washed with DMF (1.0 mL). The filtrate was purified by Prep-HPLC (Column: Welch Ultimate XB-C18, 50*250 mm, 10 μm; Mobile Phase A: 0.1% HCl, Mobile Phase B: ACN; Flow rate: 90 mL/min; Gradient: 5% B-5% B-3 min, 10% B-45% B-12 min) to afford 1-{4-[(1R,2R,3S,4R)-4-[4-amino-5-(4-benzyl-1,3-thiazol-2-yl)-2-chloropyrrolo[2,3-d]pyrimidin-7-yl]-2,3-dihydroxycyclopentyl]piperidin-1-yl}propan-2-one 2HCl salt 510 (18.9 mg, 34%) as an off-white solid. LCMS (Cortecs C18+, 50*3.0 mm, 2.7 μm, Mobile Phase A: Water/0.02% TFA, Mobile Phase B: Acetonitrile, 5%-95% B-3 min, 1.50 L/min): T_{R}=1.08 min; ES m/z [M+H]⁺: 581, 583. ¹H NMR (400 MHZ, DMSO-d₆+D₂O) 88.12 (s, 1H), 7.37-7.18 (m, 6H), 4.78-4.67 (m, 1H), 4.31 (s, 2H), 4.18 (dd, J=9.1, 6.2 Hz, 1H), 4.06 (s, 2H), 3.82-3.74 (m, 1H), 3.47-3.34 (m, 2H), 3.02-2.85 (m, 2H), 2.19 (s, 3H), 2.11-2.00 (m, 2H), 1.86-1.46 (m, 6H).

### Synthesis of (1R,2S,3R,5R)-3-[4-amino-5-(4-benzyl-1,3-thiazol-2-yl)-2-chloropyrrolo[2,3-d]pyrimidin-7-yl]-5-[1-(2,2-difluoroethyl)piperidin-4-yl]cyclopentane-1,2-diol (511)

### (1R,2S,3R,5R)-3-[4-Amino-5-(4-benzyl-1,3-thiazol-2-yl)-2-chloropyrrolo[2,3-d]pyrimidin-7-yl]-5-[1-(2,2-difluoroethyl)piperidin-4-yl]cyclopentane-1,2-diol (511)

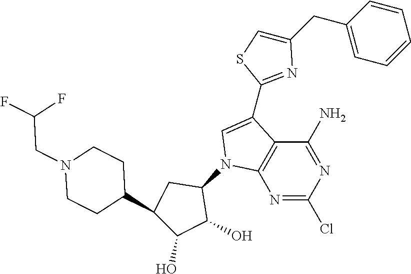 [SMILES: Nc1nc(Cl)nc2c1c(-c1nc(Cc3ccccc3)cs1)cn2[C@@H]1C[C@H](C2CCN(CC(F)F)CC2)[C@@H](O)[C@H]1O]

Into an 8 mL vial was placed a solution of (1R,2S,3R,5R)-3-[4-amino-5-(4-benzyl-1,3-thiazol-2-yl)-2-chloropyrrolo[2,3-d]pyrimidin-7-yl]-5-(piperidin-4-yl)cyclopentane-1,2-diol 110b (50.0 mg, 0.1 mmol, 1.0 eq) in CH₃CN (1.0 mL). TEA (28.9 mg, 0.3 mmol, 3.0 eq) and 2,2-difluoroethyl trifluoromethanesulfonate (40.8 mg, 0.2 mmol, 2.0 eq) were added. The mixture was stirred for 2 h at room temperature, then filtered, and the filter cake washed with CH₃CN (1.0 mL). The filtrate was purified by Prep-HPLC (Column: Welch Ultimate XB-C18, 50*250 mm, 10 μm; Mobile Phase A: 0.1% HCl, Mobile Phase B: ACN; Flow rate: 90 mL/min; Gradient: 5% B-5% B-3 min, 10% B-45% B-12 min) to afford (1R,2S,3R,5R)-3-[4-amino-5-(4-benzyl-1,3-thiazol-2-yl)-2-chloropyrrolo[2,3-d]pyrimidin-7-yl]-5-[1-(2,2-difluoroethyl)piperidin-4-yl]cyclopentane-1,2-diol 2HCl salt 511 (17.1 mg, 30%) as an off-white solid. LCMS (Cortecs C18+, 50*3.0 mm, 2.7 μm, Mobile Phase A: Water/0.02% TFA, Mobile Phase B: Acetonitrile, 5%-95% B-3 min, 1.50 L/min): T_{R}=1.11 min; ES m/z [M+H]⁺: 589, 591.1H NMR (400 MHZ, DMSO-d₆+D₂O) δ8.12 (s, 1H), 7.37-7.17 (m, 6H), 6.76-6.41 (m, 1H), 4.74 (q, J=9.1 Hz, 1H), 4.23-4.12 (m, 1H), 4.06 (s, 2H), 3.83-3.66 (m, 3H), 3.63-3.51 (m, 2H), 3.17-2.96 (m, 2H), 2.20-1.93 (m, 2H), 1.91-1.75 (m, 1H), 1.76-1.44 (m, 5H).

### Synthesis of (1R,2S,3R,5R)-3-[4-amino-2-chloro-5-(1,3-thiazol-2-yl)pyrrolo[2,3-d]pyrimidin-7-yl]-5-(1-benzylpiperidin-4-yl)cyclopentane-1,2-diol (512)

### Tert-butyl 4-[(3aR,4R,6R,6aS)-6-[4-amino-2-chloro-5-(1,3-thiazol-2-yl)pyrrolo[2,3-d]pyrimidin-7-yl]-2,2-dimethyl-tetrahydro-3aH-cvclonentald][1,3]dioxol-4-yl]nineridine-1-carboxylate (111b)

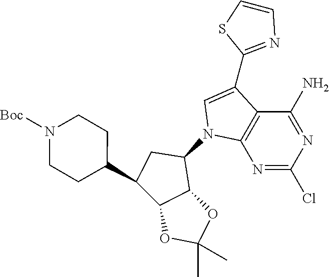 [SMILES: CC(C)(C)OC(=O)N1CCC([C@H]2C[C@@H](n3cc(-c4nccs4)c4c(N)nc(Cl)nc43)[C@@H]3OC(C)(C)O[C@@H]32)CC1]

Into an 8 mL vial was placed a solution of tert-butyl 4-[(3aR,4R,6R,6aS)-6-{4-amino-2-chloro-5-iodopyrrolo[2,3-d]pyrimidin-7-yl}-2,2-dimethyl-tetrahydro-3aH-cyclopenta[d][1,3]dioxol-4-yl]piperidine-1-carboxylate 108b (0.20 g, 0.3 mmol, 1.0 eq) in toluene (2.0 mL). 2-(Tributylstannyl)-1,3-thiazole (0.18 g, 0.48 mmol, 1.5 eq) and Pd (PPh₃) 2Cl₂ (22.7 mg, 0.032 mmol, 0.1 eq) were added, and the mixture stirred for 1 h at 100° C. under N₂ atmosphere. The resulting mixture was concentrated under reduced pressure. The residue was dissolved in DCM (1.0 mL). The residue was purified by silica gel column chromatography (30:70 THF: Petroleum ether) to afford tert-butyl 4-[(3aR,4R,6R,6aS)-6-[4-amino-2-chloro-5-(1,3-thiazol-2-yl)pyrrolo[2,3-d]pyrimidin-7-yl]-2,2-dimethyl-tetrahydro-3aH-cyclopenta[d][1,3]dioxol-4-yl]piperidine-1-carboxylate 111b (100 mg, 53%) as an off-white solid. LCMS (conditions: L-column3 ODS, 50*3.0 mm, 3.0 μm, Mobile Phase Water/5 mM NH₄HCO₃+0.05% Ammonia, Mobile Phase B: Acetonitrile/5% water, 2%-100% B-3 min, 1.50 L/min): T_{R}=2.33 min; ES m/z [M+H]⁺: 575, 577.

### (1R,2S,3R,5R)-3-[4-amino-2-chloro-5-(1,3-thiazol-2-yl)pyrrolo[2,3-d]pyrimidin-7-yl]-5-(piperidin-4-yl)cyclopentane-1,2-diol (112b)

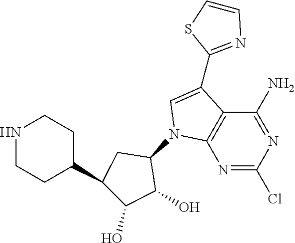 [SMILES: Nc1nc(Cl)nc2c1c(-c1nccs1)cn2[C@@H]1C[C@H](C2CCNCC2)[C@@H](O)[C@H]1O]

Into a 40 mL vial was placed a solution of tert-butyl 4-[(3aR,4R,6R,6aS)-6-[4-amino-2-chloro-5-(1,3-thiazol-2-yl)pyrrolo[2,3-d]pyrimidin-7-yl]-2,2-dimethyl-tetrahydro-3aH-cyclopenta[d][1,3]dioxol-4-yl]piperidine-1-carboxylate 111b (100 mg, 0.17 mmol, 1.0 eq) in 4M HCl in MeOH (2.0 mL). The mixture was stirred for 10 min at 60° C. The resulting mixture was concentrated under vacuum to afford (1R,2S,3R,5R)-3-[4-amino-2-chloro-5-(1,3-thiazol-2-yl)pyrrolo[2,3-d]pyrimidin-7-yl]-5-(piperidin-4-yl)cyclopentane-1,2-diol 112b (110 mg, crude) as light yellow solid. LCMS (conditions: L-column3 ODS, 50*3.0 mm, 3.0 μm, Mobile Phase Water/5 mM NH₄HCO₃+0.05% Ammonia, Mobile Phase B: Acetonitrile/5% water, 2%-100% B-3 min, 1.50 L/min): T_{R}=1.24 min; ES m/z [M+H]⁺: 435, 437.

### (1R,2S,3R,5R)-3-[4-amino-2-chloro-5-(1,3-thiazol-2-yl)pyrrolo[2,3-d]pyrimidin-7-yl]-5-(1-benzylpiperidin-4-yl)cyclopentane-1,2-diol (512)

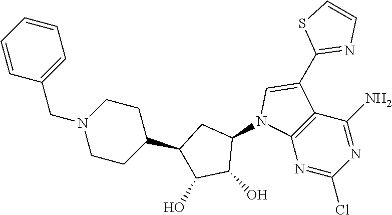 [SMILES: Nc1nc(Cl)nc2c1c(-c1nccs1)cn2[C@@H]1C[C@H](C2CCN(Cc3ccccc3)CC2)[C@@H](O)[C@H]1O]

Into 40 mL vial was placed a solution of (1R,2S,3R,5R)-3-[4-amino-2-chloro-5-(1,3-thiazol-2-yl)pyrrolo[2,3-d]pyrimidin-7-yl]-5-(piperidin-4-yl)cyclopentane-1,2-diol 112b (50 mg, 0.11 mmol, 1.0 eq) in DCM (2.0 mL). Benzaldehyde (14.64 mg, 0.13 mmol, 1.2 eq), NaBH (OAc) 3 (48.73 mg, 0.230 mmol, 2.0 eq) and DIEA (29.72 mg, 0.23 mmol, 2.0 eq) were added, and the mixture stirred for 2 h. The resulting mixture was concentrated under reduced pressure. The residue was dissolved in MeOH (2.0 mL), and purified by Prep-HPLC (Column: YMC-Actus Triart C18, 30*150 mm, 5 μm; Mobile Phase A: Water (10 mmol/L NH₄HCO₃+0.1% NH₃·H₂O), Mobile Phase B: ACN; Flow rate: 30 ml/min; Gradient: 24% B to 49% B in 7 min, 49% B; Wavelength: 220 nm;) to afford (1R,2S,3R,5R)-3-[4-amino-2-chloro-5-(1,3-thiazol-2-yl)pyrrolo[2,3-d]pyrimidin-7-yl]-5-(1-benzylpiperidin-4-yl)cyclopentane-1,2-diol 512 (5 mg, 8%) as an off-white solid. LCMS (Cortecs C18+, 50*3.0 mm, 2.7 μm, Mobile Phase A: Water/0.02% TFA, Mobile Phase B: Acetonitrile, 5%-95% B-3 min, 1.50 L/min): T_{R}=0.97 min; ES m/z [M+H]⁺: 525, 527.1H NMR (400 MHZ, DMSO-d₆) δ9.53 (s, 1H), 8.22 (s, 1H), 8.01-7.79 (m, 2H), 7.71-7.57 (m, 1H), 7.44-7.17 (m, 5H), 4.89-4.69 (m, 2H), 4.65-4.44 (m, 1H), 4.24-4.08 (m, 1H), 3.83-3.68 (m, 1H), 3.44 (s, 2H), 2.95-2.71 (m, 2H), 2.12-1.98 (m, 1H), 1.98-1.75 (m, 3H), 1.77-1.47 (m, 3H), 1.39-1.02 (m, 3H).

### Synthesis of (1R,2S,3R,5R)-3-(4-amino-2-chloro-5-(thiazol-2-yl)-7H-pyrrolo[2,3-d]pyrimidin-7-yl)-5-(1-phenethylpiperidin-4-yl)cyclopentane-1,2-diol (513)

### (1R,2S,3R,5R)-3-(4-amino-2-chloro-5-(thiazol-2-yl)-7H-pyrrolo[2,3-d]pyrimidin-7-yl)-5-(1-phenethylpiperidin-4-yl)cyclopentane-12-diol (513)

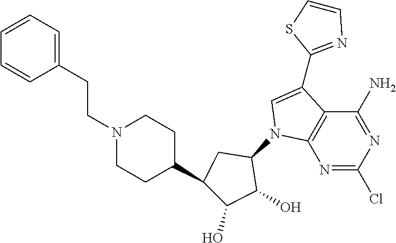 [SMILES: Nc1nc(Cl)nc2c1c(-c1nccs1)cn2[C@@H]1C[C@H](C2CCN(CCc3ccccc3)CC2)[C@@H](O)[C@H]1O]

Into a 2 mL vial was added a solution of (1R,2S,3R,5R)-3-[4-amino-2-chloro-5-(1,3-thiazol-2-yl)pyrrolo[2,3-d]pyrimidin-7-yl]-5-(piperidin-4-yl)cyclopentane-1,2-diol 112b (40 mg, 0.1 mmol, 1 eq) in DCM (0.5 mL) and phenylacetaldehyde (16.57 mg, 0.1 mmol, 1.5 eq). NaBH (OAc) 3 (38.98 mg, 0.2 mmol,2.0 eq) was added and the mixture stirred for 30 min. The resulting mixture was concentrated under reduced pressure. The residue was purified by reverse phase flash with the following conditions (Column: Welch Utimate AQ-C18, 50*250 mm*10 μm; Mobile Phase A: Water (10 mmol/L NH₄HCO₃+0.1% NH₃: H₂O), Mobile Phase B: ACN; Flow rate: 80 mL/min; Gradient: 5% B to 65% B in 15 min, 65% B; Wavelength: 220 nm) to afford (1R,2S,3R,5R)-3-(4-amino-2-chloro-5-(thiazol-2-yl)-7H-pyrrolo[2,3-d]pyrimidin-7-yl)-5-(1-phenethylpiperidin-4-yl)cyclopentane-1,2-diol 513 (10.4 mg, 20%) as a white solid. LCMS (conditions: ZORBAX SB-Aq, 50*4.6 mm, 1.8 μm; Mobile Phase A: Water/0.02% TFA, Mobile Phase B: Acetonitrile; 5%-95% B-5 min, 1.50 mL/min); T_{R}=2.33 min; ES m/z [M+H]⁺: 539,541. ¹H NMR (400 MHZ, DMSO-d₆+D₂O) δ 8.19 (s, 1H), 7.82 (d, J=3.4 Hz, 1H), 7.62 (d, J=3.4 Hz, 1H), 7.31-7.23 (m, 2H), 7.23-7.13 (m, 3H), 4.82-4.70 (m, 1H), 4.17 (dd, J=9.5, 6.3 Hz, 1H), 3.78 (dd, J=6.4, 3.3 Hz, 1H), 2.97-2.90 (m, 2H), 2.76-2.68 (m, 2H), 2.51-2.43 (m, 2H), 2.10-1.99 (m, 1H), 1.99-1.80 (m, 3H), 1.71-1.53 (m, 3H), 1.35-1.20 (m, 3H).

### Synthesis of (1R,2S,3R,5R)-3-[4-amino-2-chloro-5-(1,3-thiazol-2-yl)pyrrolo[2,3-d]pyrimidin-7-yl]-5-{1-[2-(pyridin-4-yl)ethyl]piperidin-4-yl}cyclopentane-1,2-diol (514)

### (1R,2S,3R,5R)-3-[4-amino-2-chloro-5-(1,3-thiazol-2-yl)pyrrolo[2,3-d]pyrimidin-7-yl]-5-{1-[2-(pyridin-4-yl)ethyl]piperidin-4-yl}cyclopentane-1,2-diol (514)

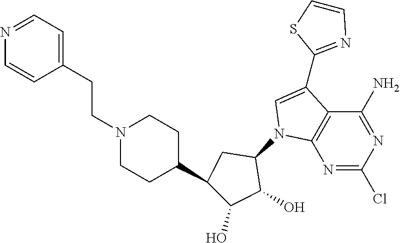 [SMILES: Nc1nc(Cl)nc2c1c(-c1nccs1)cn2[C@@H]1C[C@H](C2CCN(CCc3ccncc3)CC2)[C@@H](O)[C@H]1O]

Into a 40 mL vial was placed a solution of (1R,2S,3R,5R)-3-[4-amino-2-chloro-5-(1,3-thiazol-2-yl)pyrrolo[2,3-d]pyrimidin-7-yl]-5-(piperidin-4-yl)cyclopentane-1,2-diol 112b (50.0 mg, 0.1 mmol, 1.0 eq) in dioxane (1.0 mL). Na₂CO₃ (24.4 mg, 0.2 mmol, 2.0 eq) and 2-(pyridin-4-yl)ethyl methanesulfonate (46.3 mg, 0.2 mmol, 2.0 eq) [WO2014109414]were added, and the mixture stirred for 16 h at 80° C. The resulting mixture was filtered, and the filter cake washed with MeOH (1.0 mL). The filtrate was purified by Prep-HPLC (Column: YMC-Actus Triart C18, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 25% B to 60% B over 10 min, 55% B; Wavelength: 254 nm) to afford (1R,2S,3R,5R)-3-[4-amino-2-chloro-5-(1,3-thiazol-2-yl)pyrrolo[2,3-d]pyrimidin-7-yl]-5-{1-[2-(pyridin-4-yl)ethyl]piperidin-4-yl}cyclopentane-1,2-diol 514 (9 mg, 14%) as an off-white solid. LCMS (ZORBAX SB-Aq, 50*4.6 mm, 1.8 μm, Mobile Phase A: Water/0.02% TFA, Mobile Phase B: Acetonitrile, 5%-95% B-3 min, 1.50 L/min): T_{R}=1.15 min; ES m/z [M+H]⁺: 540, 542. ¹H NMR (400 MHz, DMSO-d₆) δ9.53 (br, s, 1H), 8.49-8.37 (m, 2H), 8.23 (s, 1H), 7.94 (br, s, 1H), 7.83 (d, J=3.3 Hz, 1H), 7.65 (d, J=3.4 Hz, 1H), 7.26 (d, J=5.1 Hz, 2H), 4.84 (d, J=6.8 Hz, 1H), 4.82-4.73 (m, 1H), 4.59 (d, J=4.7 Hz, 1H), 4.25-4.14 (m, 1H), 3.83-3.71 (m, 1H), 3.04-2.86 (m, 2H), 2.81-2.71 (m, 2H), 2.57-2.52 (m, 2H), 2.10-1.99 (m, 1H), 1.99-1.81 (m, 3H), 1.72-1.53 (m, 3H), 1.38-1.12 (m, 3H).

### Synthesis of (1R,2S,3)-3-[5-(4-benzyl-1,3-thiazol-2-yl)-2-chloropyrrolo[2,3-d]pyrimidin-7-yl]-5-(1-methylpiperidin-3-yl)cyclopentane-1,2-diol (515)

### tert-butyl 3-[(3aR,4R,6aR)-2,2-dimethyl-6-oxo-tetrahydrocyclopenta[d][1,3]dioxol-4-yl]piperidine-1-carboxylate (113b)

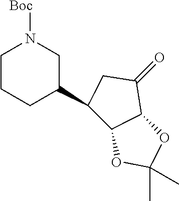 [SMILES: CC(C)(C)OC(=O)N1CCCC([C@H]2CC(=O)[C@@H]3OC(C)(C)O[C@H]23)C1]

Into a 250 ml vial was placed a solution of (3aR,6aR)-2,2-dimethyl-3aH,6aH-cyclopenta[d][1,3]dioxol-4-one (1.50 g, 9.7 mmol, 1.0 eq) in DMF (150.0 mL). 1-(Tert-butoxycarbonyl)piperidine-3-carboxylic acid (3.35 g, 14.5 mmol, 1.5 eq), Ir[dF(CF₃)ppy]₂ (dtbpy) PF₆ (0.55 g, 0.5 mmol, 0.05 eq) and K₂HPO₄ (3.39 g, 19.5 mmol, 2.0 eq) were added. The mixture was stirred for 48 h at room temperature under blue LED (45 W) light. The resulting mixture was diluted with water (300.0 mL) and extracted with AcOEt (3×100 mL). The combined organic layers were washed with brine, dried over anhydrous Na₂SO₄. After filtration, the filtrate was concentrated under reduced pressure. The residue was dissolved in DCM (10.0 mL). The residue was purified by silica gel column chromatography (75:25 Petroleum ether: THF) to afford tert-butyl 3-[(3aR,4R,6aR)-2,2-dimethyl-6-oxo-tetrahydrocyclopenta[d][1,3]dioxol-4-yl]piperidine-1-carboxylate 113b (1.80 g, 54%) as light yellow oil. LCMS (conditions: L-column3 C18, 50*3.0 mm, 3.0 μm, Mobile Phase A: Water/5 mM NH₄HCO₃, Mobile Phase B: Methanol, 2%-100% B-3 min, 1.50 L/min): T_{R}=2.45 min; ES m/z [M+H]⁺: 340.

### Tert-butyl 3-[(3aR,4R,6S,6aS)-6-hydroxy-2,2-dimethyl-tetrahydro-3aH-cyclopenta[d][1,3]dioxol-4-yl]piperidine-1-carboxylate (114b)

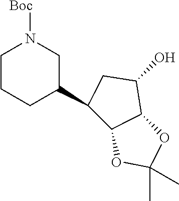 [SMILES: CC(C)(C)OC(=O)N1CCCC([C@H]2C[C@H](O)[C@@H]3OC(C)(C)O[C@@H]32)C1]

Into a 250 mL flask was placed a solution of tert-butyl 3-[(3aR,4R,6aR)-2,2-dimethyl-6-oxo-tetrahydrocyclopenta[d][1,3]dioxol-4-yl]piperidine-1-carboxylate 113b (1.70 g, 5.1 mmol, 1.0 eq) in MeOH (17.0 mL), then NaBH₄ (0.19 g, 5.1 mmol, 1.0 eq) was added. The resulting mixture was stirred for 30 min at room temperature, then concentrated under vacuum. The residue was dissolved in water (30.0 ml) and extracted with AcOEt (3×10.0 mL). The combined organic layers were washed with brine and dried over anhydrous Na₂SO₄. After filtration, the filtrate was concentrated under reduced pressure to afford tert-butyl 3-[(3aR,4R,6S,6aS)-6-hydroxy-2,2-dimethyl-tetrahydro-3aH-cyclopenta[d][1,3]dioxol-4-yl]piperidine-1-carboxylate 114b (1.6 g, 93%) as light yellow oil. LCMS (Conditions: L-column3 ODS, 50*3.0 mm, 3.0 μm, Mobile Phase A: Water/5 mM NH₄HCO₃+0.05% Ammonia, Mobile Phase B: Acetonitrile/5% water, 5%-95% B-3 min, 1.50 L/min): T_{R}=1.76 min; ES m/z [M+H]⁺: 342.

### Tert-butyl 3-[(3aR,4R,6R,6aS)-6-{2-chloro-5-iodopyrrolo[2,3-d]pyrimidin-7-yl}-2,2-dimethyl-tetrahydro-3aH-cyclopenta[d][1,3]dioxol-4-yl]piperidine-1-carboxylate (115b)

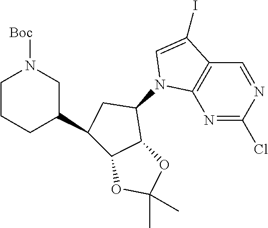 [SMILES: CC(C)(C)OC(=O)N1CCCC([C@H]2C[C@@H](n3cc(I)c4cnc(Cl)nc43)[C@@H]3OC(C)(C)O[C@@H]32)C1]

Into a 250 ml flask was placed a solution of tert-butyl 3-[(3aR,4R,6S,6aS)-6-hydroxy-2,2-dimethyl-tetrahydro-3aH-cyclopenta[d][1,3]dioxol-4-yl]piperidine-1-carboxylate 114b (1.17 g, 3.4 mmol, 1.2 eq) in toluene (80.0 mL). 2-Chloro-5-iodo-7H-pyrrolo[2,3-d]pyrimidine (0.80 g, 2.9 mmol, 1.0 eq) and PPh₃ (1.50 g, 5.7 mmol, 2.0 eq) were added. The mixture was cooled to 0° C. and DBAD (1.31 g, 5.7 mmol, 2.0 eq) in toluene (20.0 mL) was added dropwise over 2 min at 0° C. The resulting mixture was stirred for 1.5h at 50° C., then concentrated under reduced pressure. The residue was dissolved in DCM (2.0 mL) and purified by silica gel column chromatography (78:22 Petroleum ether/AcOE) to afford tert-butyl 3-[(3aR,4R,6R,6aS)-6-{2-chloro-5-iodopyrrolo[2,3-d]pyrimidin-7-yl}-2,2-dimethyl-tetrahydro-3aH-cyclopenta[d][1,3]dioxol-4-yl]piperidine-1-carboxylate 115b (800 mg, 46%) as an off-white solid. LCMS (Conditions: L-column3 ODS, 50*3.0 mm, 3.0 μm, Mobile Phase A: Water/5 mM NH₄HCO₃+0.05% Ammonia, Mobile Phase B: Acetonitrile/5% water, 5%-95% B-3 min, 1.50 L/min): T_{R}=2.42 min; ES m/z [M+H]⁺: 603, 605.

### Tert-butyl 3-[(3aR,6R,6aS)-6-[5-(4-benzyl-1,3-thiazol-2-yl)-2-chloropyrrolo[2,3-d]pyrimidin-7-yl]-2,2-dimethyl-tetrahydro-3aH-cyclopenta[d][1,3]dioxol-4-yl]piperidine-1-carboxylate (116b)

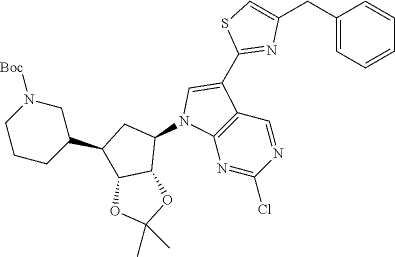 [SMILES: CC(C)(C)OC(=O)N1CCCC([C@H]2C[C@@H](n3cc(-c4nc(Cc5ccccc5)cs4)c4cnc(Cl)nc43)[C@@H]3OC(C)(C)O[C@@H]32)C1]

Into a 40 mL vial was placed a solution of tert-butyl 3-[(3aR,6R,6aS)-6-{2-chloro-5-iodopyrrolo[2,3-d]pyrimidin-7-yl}-2,2-dimethyl-tetrahydro-3aH-cyclopenta[d][1,3]dioxol-4-y]piperidine-1-carboxylate 115b (0.15 g, 0.249 mmol, 1.0 eq) in toluene (2.0 ml). Pd (PPh₃) 2Cl₂ (17.46 mg, 0.025 mmol, 0.1 eq) and K₃PO₄ (0.16 g, 0.7 mmol, 3.0 eq) were added followed by (4-benzyl-1,3-thiazol-2-yl)(chloro) zinc (0.32 g, 1.2 mmol, 5.0 eq) dropwise. The resulting mixture was stirred at 60° C. overnight. The reaction was quenched by saturated NH₄Cl solution (20.0 mL) at room temperature. The resulting mixture was extracted with AcOEt (3×10 mL). The combined organic layers were washed with brine, dried over anhydrous Na₂SO₄. After filtration, the filtrate was concentrated under reduced pressure. The residue was purified by silica gel column chromatography (70:30 Petroleum ether: AcOE) to afford tert-butyl 3-[(3aR,6R,6aS)-6-[5-(4-benzyl-1,3-thiazol-2-yl)-2-chloropyrrolo[2,3-d]pyrimidin-7-yl]-2,2-dimethyl-tetrahydro-3aH-cyclopenta[d][1,3]dioxol-4-yl]piperidine-1-carboxylate 116b (130 mg, 80%) as a light yellow solid. LCMS (Conditions: L-column3 ODS, 50*3.0 mm, 3.0 μm, Mobile Phase A: Water/5 mM NH₄HCO₃+0.05% Ammonia, Mobile Phase B: Acetonitrile/5% water, 5%-95% B-3 min, 1.50 L/min): T_{R}=2.44 min; ES m/z [M+H]⁺: 650, 652.

### (1R,2S,3R)-3-[5-(4-benzyl-1,3-thiazol-2-yl)-2-chloropyrrolo[2,3-d]pyrimidin-7-yl]-5-(piperidin-3-yl)cyclopentane-1,2-diol (117b)

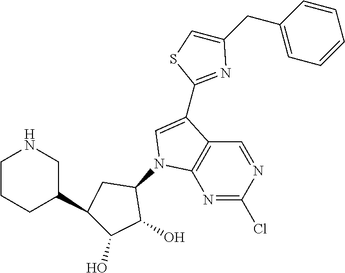 [SMILES: O[C@@H]1[C@H](O)[C@@H](C2CCCNC2)C[C@H]1n1cc(-c2nc(Cc3ccccc3)cs2)c2cnc(Cl)nc21]

Into a 40 mL vial was placed a solution of tert-butyl 3-[(3aR,6R,6aS)-6-[5-(4-benzyl-1,3-thiazol-2-yl)-2-chloropyrrolo[2,3-d]pyrimidin-7-yl]-2,2-dimethyl-tetrahydro-3aH-cyclopenta[d][1,3]dioxol-4-yl]piperidine-1-carboxylate 116b (0.15 g, 0.2 mmol, 1.0 eq) in 4M HCl in MeOH (2.0 mL). The mixture was stirred for 10 min at 60° C. The resulting mixture was concentrated under reduced pressure to afford crude product (1R,2S,3R)-3-[5-(4-benzyl-1,3-thiazol-2-yl)-2-chloropyrrolo[2,3-d]pyrimidin-7-yl]-5-(piperidin-3-yl)cyclopentane-1,2-diol 117b (50 mg) as a light yellow solid. The mixture was used in the next step directly without further purification. LCMS (Conditions: L-column3 ODS, 50*3.0 mm, 3.0 μm, Mobile Phase A: Water/5 mM NH₄HCO₃+0.05% Ammonia, Mobile Phase B: Acetonitrile/5% water, 5%-95% B-3 min, 1.50 L/min): T_{R}=1.58 min; ES m/z [M+H]⁺: 510, 512.

### (1R,2S,3R)-3-[5-(4-benzyl-1,3-thiazol-2-yl)-2-chloropyrrolo[2,3-d]pyrimidin-7-yl]-5-(1-methylpiperidin-3-yl)cyclopentane-1,2-diol (515)

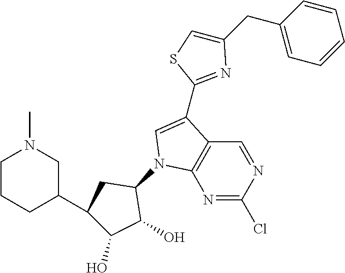 [SMILES: CN1CCCC([C@H]2C[C@@H](n3cc(-c4nc(Cc5ccccc5)cs4)c4cnc(Cl)nc43)[C@H](O)[C@@H]2O)C1]

Into a 40 mL vial was added a solution of (1R,2S,3R)-3-[5-(4-benzyl-1,3-thiazol-2-yl)-2-chloropyrrolo[2,3-d]pyrimidin-7-yl]-5-(piperidin-3-yl)cyclopentane-1,2-diol 117b (50.00 mg, 0.1 mmol, 1.0 eq) in DCM (0.5 mL). DIEA (25.34 mg, 0.2 mmol, 2.0 eq) and 30% HCHO in H₂O (18 mg mg, 0.2 mmol, 2.0 eq) were added, followed by NaBH (OAc) 3 (42 mg, 0.2 mmol, 2.0 eq). The resulting mixture was stirred for 30 min, then concentrated under reduced pressure. The mixture was dissolved with MeOH (3.0 mL) and purified by Prep-HPLC (Column: Welch Utimate AQ-C18, 50*250 mm*10 μm; Mobile Phase A: Water (10 mmol/L NH₄HCO₃+0.1% NH₃·H₂O), Mobile Phase B: ACN; Flow rate: 80 mL/min; Gradient: 5% B to 65% B in 15 min) to afford (1R,2S,3R)-3-[5-(4-benzyl-1,3-thiazol-2-yl)-2-chloropyrrolo[2,3-d]pyrimidin-7-yl]-5-(1-methylpiperidin-3-yl)cyclopentane-1,2-diol 515 (30 mg, 58%) as a white solid. LCMS (Cortecs C18+, 50*3.0 mm, 2.7 μm, Mobile Phase A: Water/0.02% TFA, Mobile Phase B: Acetonitrile, 2%-95% B-3 min, 1.50 L/min): T_{R}=1.07, 1.12 min; ES m/z [M+H]⁺: 524, 526 ¹H NMR (400 MHZ, DMSO-d₆) δ9.31 (s, 1H), 8.66-8.58 (m, 1H), 7.39-7.29 (m, 5H), 7.28-7.19 (m, 1H), 4.98-4.82 (m, 2H), 4.70-4.59 (m, 1H), 4.27-4.18 (m, 1H), 4.14 (s, 2H), 3.85-3.74 (m, 1H), 3.03-2.92 (m, 1H), 2.75-2.61 (m, 1H), 2.18-2.09 (m, 3H), 2.09-2.00 (m, 1H), 1.96-1.51 (m, 7H).

### Synthesis of (1R,2S,3R,5R)-3-(5-(4-benzylthiazol-2-yl)-2-chloro-7H-pyrrolo[2,3-d]pyrimidin-7-yl)-5-(1-(cyclopropylmethyl)piperidin-3-yl)cyclopentane-1,2-diol (516)

### (1R,2S,3R,5R)-3-(5-(4-benzylthiazol-2-yl)-2-chloro-7H-pyrrolo[2,3-d]pyrimidin-7-yl)-5-(1-(cyclopropylmethyl)piperidin-3-yl)cyclopentane-1,2-diol (516)

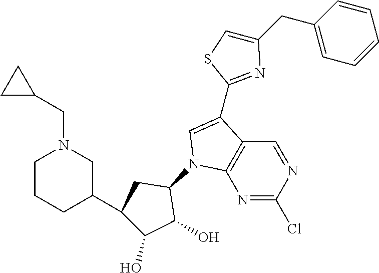 [SMILES: O[C@@H]1[C@H](O)[C@@H](C2CCCN(CC3CC3)C2)C[C@H]1n1cc(-c2nc(Cc3ccccc3)cs2)c2cnc(Cl)nc21]

Into a 40 mL vial was added a solution of (1R,2S,3R)-3-[5-(4-benzyl-1,3-thiazol-2-yl)-2-chloropyrrolo[2,3-d]pyrimidin-7-yl]-5-(piperidin-3-yl)cyclopentane-1,2-diol 117b (50 mg, 0.1 mmol, 1.0 eq) in DCM (0.5 mL). DIEA (25 mg, 0.2 mmol, 2.0 eq) and cyclopropanecarbaldehyde (14 mg, 0.2 mmol, 2.0 eq) were added, followed by NaBH (OAc) 3 (42 mg, 0.2 mmol, 2.0 eq). The resulting mixture was stirred for 30 min then concentrated under reduced pressure. The crude product was purified by reverse phase flash with the following conditions (Column: Welch Ultimate AQ-C18, 50*250 mm*10 μm; Mobile Phase A: Water (10 mmol/L NH₄HCO₃+0.1% NH₃: H₂O), Mobile Phase B: ACN; Flow rate: 80 mL/min; Gradient: 5% B to 65% B in 15 min, 65% B; Wavelength: 220 nm; T_{R}=14.9 min) to afford (1R,2S,3R,5R)-3-(5-(4-benzylthiazol-2-yl)-2-chloro-7H-pyrrolo[2,3-d]pyrimidin-7-yl)-5-(1-(cyclopropylmethyl)piperidin-3-yl)cyclopentane-1,2-diol 516 (5.5 mg, 10%) as a white solid. LCMS (conditions: Cortecs C18+,50*3.0 mm, 2.7 μm, Mobile Phase A: Water/0.02% TFA, Mobile Phase B: Acetonitrile, 5%-95% B-3 min, 1.50 L/min), T_{R}=1.14, 1.16 min; ES m/z [M+H]⁺: 564, 566. ¹H NMR (400 MHZ, DMSO-d₆) δ9.31 (s, 1H), 8.86 (s, 0.5H), 8.59 (s, 0.5H), 7.40-7.30 (m, 5H), 7.26-7.19 (m, 1H), 4.96-4.86 (m, 2H), 4.68-4.61 (m, 1H), 4.27-4.17 (m, 1H), 4.15 (s, 2H), 3.83-3.79 (m, 1H), 3.18-3.10 (m, 0.5H), 2.97-2.91 (m, 0.5H), 2.90-2.83 (m, 1H), 2.31-2.03 (m, 3H), 2.03-1.53 (m, 7H), 1.50-1.44 (m, 1H), 0.90-0.79 (m, 2H), 0.50-0.37 (m, 2H), 0.11-0.05 (m, 2H).

### Synthesis of (1R,2S,3R,5R)-3-(5-(4-benzylthiazol-2-yl)-2-chloro-7H-pyrrolo[2,3-d]pyrimidin-7-yl)-5-(1-(2,2,2-trifluoroethyl)-313-piperidin-3-yl)cyclopentane-1,2-diol (517)

### (1R,2S,3R,5R)-3-(5-(4-Benzylthiazol-2-yl)-2-chloro-7H-pyrrolo[2,3-d]pyrimidin-7-yl)-5-(1-(2,2,2-trifluoroethyl)-313-piperidin-3-yl)cyclopentane-1,2-diol (517)

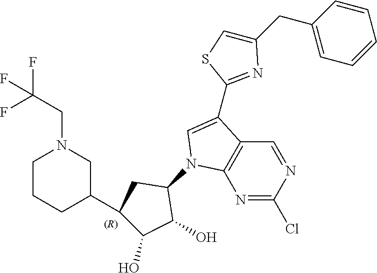

Into an 8 mL vial was added a solution of (1R,2S,3R)-3-[5-(4-benzyl-1,3-thiazol-2-yl)-2-chloropyrrolo[2,3-d]pyrimidin-7-yl]-5-(piperidin-3-yl)cyclopentane-1,2-diol 117b (50 mg, 0.1 mmol, 1.0 eq) in DCM (1.0 mL), followed by DIEA (25.34 mg, 0.2 mmol, 2.0 eq) and trifluoroacetaldehyde (19.22 mg, 0.2 mmol, 2.0 eq), and finally NaBH (OAc) 3 (41.55 mg, 0.2 mmol, 2.0 eq). The reaction was stirred for 30 min, then concentrated under reduced pressure. The residue was purified by reverse phase flash with the following conditions (Column: Welch Utimate AQ-C18, 50*250 mm*10 μm; Mobile Phase A: Water (10 mmol/L NH₄HCO₃+0.1% NH₃: H₂O), Mobile Phase B: ACN; Flow rate: 80 mL/min; Gradient: 5% B to 65% B in 15 min, 65% B; Wavelength: 220 nm; T_{R}=14.9 min) to afford (1R,2S,3R,5R)-3-(5-(4-benzylthiazol-2-yl)-2-chloro-7H-pyrrolo[2,3-d]pyrimidin-7-yl)-5-(1-(2,2,2-trifluoroethyl)-313-piperidin-3-yl)cyclopentane-1,2-diol 517 (5.7 mg, 9%) as a yellow solid. LCMS (conditions: Cortecs C18+, 50*3.0 mm, 2.7 μm, Mobile Phase A: Water/0.02% TFA, Mobile Phase B: Acetonitrile, 5%-95% B-5 min,1.50 mL/min): T_{R}=2.47 min; ES m/z [M+H]⁺: 592, 594.1H NMR (400 MHZ, DMSO-d₆+D₂O) δ9.31 (s, 1H), 8.57 (d, J=8.2 Hz, 1H), 7.40-7.27 (m, 5H), 7.26-7.18 (m, 1H), 4.96-4.85 (m, 1H), 4.28-4.17 (m, 2H), 4.14-4.05 (m, 3H), 3.90-3.78 (m, 1H), 3.70-3.62 (m, 1H), 3.38-3.30 (m, 1H), 3.08-2.97 (m, 1H), 2.92-2.82 (m, 1H), 2.28-1.57 (m, 7H), 1.27-1.13 (m, 1H).

### Synthesis of (1R,2S,3R,5R)-3-(5-(4-benzylthiazol-2-yl)-2-chloro-7H-pyrrolo[2,3-d]pyrimidin-7-yl)-5-(1-phenethylpiperidin-3-yl)cyclopentane-1,2-diol (518)

### (1R,2S,3R,5R)-3-(5-(4-benzylthiazol-2-yl)-2-chloro-7H-pyrrolo[2,3-d]pyrimidin-7-yl)-5-(1-phenethylpiperidin-3-yl)cyclopentane-1,2-diol (518)

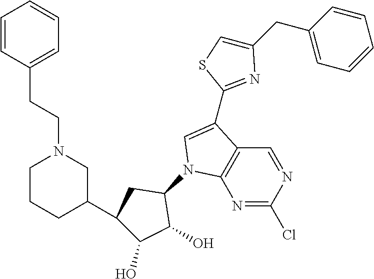 [SMILES: O[C@@H]1[C@H](O)[C@@H](C2CCCN(CCc3ccccc3)C2)C[C@H]1n1cc(-c2nc(Cc3ccccc3)cs2)c2cnc(Cl)nc21]

Into a 40 mL vial was added a solution of (1R,2S,3R)-3-[5-(4-benzyl-1,3-thiazol-2-yl)-2-chloropyrrolo[2,3-d]pyrimidin-7-yl]-5-(piperidin-3-yl)cyclopentane-1,2-diol 117b (50.00 mg, 0.1 mmol, 1.0 eq) in DCM (1.0 mL), followed by DIEA (25.34 mg, 0.2 mmol, 2.0 eq), phenylacetaldehyde (35.34 mg, 0.3 mmol, 3.0 eq) and NaBH (OAc) 3 (41.55 mg, 0.2 mmol, 2.0 eq). The resulting mixture was stirred for 30 min at room temperature, then concentrated under reduced pressure. The residue was purified by reverse phase flash with the following conditions (Column: Welch Utimate AQ-C18, 50*250 mm*10 μm; Mobile Phase A: Water (10 mmol/L NH₄HCO₃+0.1% NH₃: H₂O), Mobile Phase B: ACN; Flow rate: 80 mL/min; Gradient: 5% B to 65% B in 15 min, 65% B; Wavelength: 220 nm) to afford (1R,2S,3R,5R)-3-(5-(4-benzylthiazol-2-yl)-2-chloro-7H-pyrrolo[2,3-d]pyrimidin-7-yl)-5-(1-phenethylpiperidin-3-yl)cyclopentane-1,2-diol 518 (5.4 mg, 8%) as a yellow solid. LCMS (conditions: Cortecs C18+,50*3.0 mm,2.7 μm, Mobile Phase A: Water/0.02% TFA, Mobile Phase B: Acetonitrile, 5%-95% B-5 min, 1.50 mL/min): T_{R}=2.02 min; ES m/z [M+H]⁺: 614, 616. ¹H NMR (400 MHZ, DMSO-d₆+D₂O) δ 9.31 (s, 1H), 8.63-8.50 (m, 1H), 7.47-7.11 (m, 11H), 4.97-4.86 (m, 1H), 4.30-4.17 (m, 1H), 4.14 (s, 2H), 3.94-3.84 (m, 1H), 3.76-3.67 (m, 1H), 3.50-3.42 (m, 1H), 3.33-3.21 (m, 2H), 3.16-3.00 (m, 2H), 2.92-2.83 (m, 1H), 2.81-2.70 (m, 1H), 2.24-1.60 (m, 7H), 1.27-1.17 (m, 1H).

### Synthesis of (1R,2S,3R,5R)-3-{4-amino-5-[1-(benzenesulfonyl)pyrazol-3-yl]-2-chloropyrrolo[2,3-d]pyrimidin-7-yl}-5-[1-(2-phenylethyl)piperidin-4-yl]cyclopentane-1,2-diol (519)

### 4-[(3aR,4R,6R,6aS)-6-{5-Bromo-2,4-dichloropyrrolo[2,3-d]pyrimidin-7-yl}-2,2-dimethyl-tetrahydro-3aH-cyclopenta[d][1,3]dioxol-4-yl]-1-(2-phenylethyl)piperidine (118b)

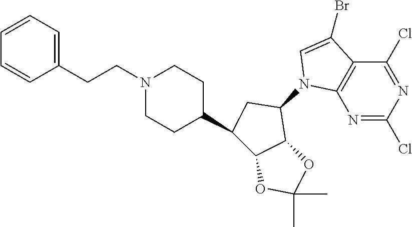 [SMILES: CC1(C)O[C@@H]2[C@H](O1)[C@@H](C1CCN(CCc3ccccc3)CC1)C[C@H]2n1cc(Br)c2c(Cl)nc(Cl)nc21]

Into a 40 mL vial was placed a solution of (1R,2S,3R,5R)-3-{5-bromo-2,4-dichloropyrrolo[2,3-d]pyrimidin-7-yl}-5-(piperidin-4-yl)cyclopentane-1,2-diol 70b (0.25 g, 0.5 mmol, 1.0 equiv) in DCM (4.0 mL). Phenylacetaldehyde (0.10 g, 0.8 mmol, 1.5 equiv), DIEA (0.14 g, 1.1 mmol, 2.0 equiv) and NaBH (OAc) 3 (0.23 g, 1.1 mmol, 2.0 equiv) were added. The mixture was stirred for 2h then concentrated under reduced pressure. The residue was dissolved in acetone (2.0 mL) and 2,2-dimethoxypropane (2.0 mL) and TsOH (0.24 g, 1.4 mmol, 2.5 equiv) were added. The mixture was stirred for 2 h then quenched with saturated aqueous NaHCO₃. The resulting mixture was extracted with AcOEt (3×3.0 mL). The combined organic layers were washed with brine, dried over anhydrous Na₂SO₄. After filtration, the filtrate was concentrated under reduced pressure and dissolved in DCM (2.0 mL). The residue was purified by silica gel column chromatography (70:30 n-hexane: THF) to afford 4-[(3aR,4R,6R,6aS)-6-{5-bromo-2,4-dichloropyrrolo[2,3-d]pyrimidin-7-yl}-2,2-dimethyl-tetrahydro-3aH-cyclopenta[d][1,3]dioxol-4-yl]-1-(2-phenylethyl)piperidine 118b (100 mg, 30%) as a light yellow solid. LCMS (conditions: L-column3 ODS, 50*3.0 mm, 3.0 μm, Mobile Phase Water/5 mM NH₄HCO₃+0.05% Ammonia, Mobile Phase B: Acetonitrile/5% water, 2%-100% B-3 min, 1.50 L/min): T_{R}=2.55 min; ES m/z [M+H]⁺: 593, 595.

### (1R,2S,3R,5R)-3-{4-amino-5-bromo-2-chloropyrrolo[2,3-d]pyrimidin-7-yl}-5-[1-(2-phenylethyl)piperidin-4-yl]cyclopentane-1,2-diol (119b)

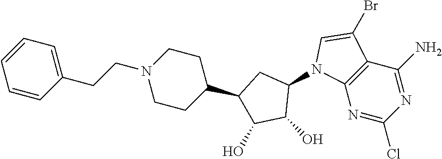 [SMILES: Nc1nc(Cl)nc2c1c(Br)cn2[C@@H]1C[C@H](C2CCN(CCc3ccccc3)CC2)[C@@H](O)[C@H]1O]

Into an 8 mL vial was placed a solution of 4-[(3aR,4R,6R,6aS)-6-{5-bromo-2,4-dichloropyrrolo[2,3-d]pyrimidin-7-yl}-2,2-dimethyl-tetrahydro-3aH-cyclopenta[d][1,3]dioxol-4-yl]-1-(2-phenylethyl)piperidine 118b (0.20 g, 0.36 mmol, 1.0 equiv) in 7M NH₃ in MeOH (2.0 mL). The mixture was stirred for 2 h at 80° C. The resulting mixture was concentrated under reduced pressure and dissolved in 4M HCl in MeOH (2.0 mL), then stirred for 1 h at 60° C. The resulting mixture was concentrated under vacuum. The residue was dissolved in MeOH and basified to pH>8 with TEA. The mixture was purified by Prep-HPLC (Column: Welch Xtimate C18 ExRS, 250 mm, 10 μm; Mobile Phase A: Water (0.05% NH₃·H₂O), Mobile Phase B: ACN; Flow rate: 90 mL/min; Gradient: 15% B to 60% B over 10 min, 90% B; Wavelength: 254 nm; T_{R}=7 min) to afford (1R,2S,3R,5R)-3-{4-amino-5-bromo-2-chloropyrrolo[2,3-d]pyrimidin-7-yl}-5-[1-(2-phenylethyl)piperidin-4-yl]cyclopentane-1,2-diol 119b (100 mg, 55%) as an off-white solid. LCMS (conditions: L-column3 ODS, 50*3.0 mm, 3.0 μm, Mobile Phase Water/5 mM NH₄HCO₃+0.05% Ammonia, Mobile Phase B: Acetonitrile/5% water, 2%-100% B-3 min, 1.50 L/min): T_{R}=1.78 min; ES m/z [M+H]⁺: 534, 536.

### (1R,2S,3R,5R)-3-{4-Amino-5-[1-(benzenesulfonyl)pyrazol-3-yl]-2-chloropyrrolo[2,3-d]pyrimidin-7-yl}-5-[1-(2-phenylethyl)piperidin-4-yl]cyclopentane-1,2-diol (519)

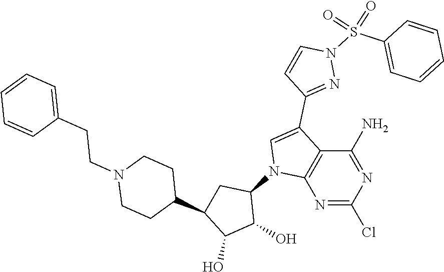 [SMILES: Nc1nc(Cl)nc2c1c(-c1ccn(S(=O)(=O)c3ccccc3)n1)cn2[C@@H]1C[C@H](C2CCN(CCc3ccccc3)CC2)[C@@H](O)[C@H]1O]

Into a 40 mL vial was placed a solution of (1R,2S,3R,5R)-3-{4-amino-5-bromo-2-chloropyrrolo[2,3-d]pyrimidin-7-yl}-5-[1-(2-phenylethyl)piperidin-4-yl]cyclopentane-1,2-diol 119b (50.0 mg, 0.1 mmol, 1.0 equiv) in H₂O (0.05 mL) and 1,4-dioxane (0.45 mL). 1-(Benzenesulfonyl)pyrazol-3-ylboronic acid (35.3 mg, 0.14 mmol, 1.5 equiv), Pd (dtbpf) Cl₂ (6.1 mg, 0.009 mmol, 0.1 equiv) and K₃PO₄ (0.40 mg, 0.19 mmol, 2.0 equiv) were added. The mixture was stirred for 1 h at 80° C. under N₂ atmosphere. The resulting mixture was filtered, and the filtrate purified by Prep-HPLC (Column: Welch Xtimate C18 ExRS, 250 mm, 10 μm; Mobile Phase A: Water (0.05% NH₃·H₂O), Mobile Phase B: ACN; Flow rate: 90 mL/min; Gradient: 20% B to 60% B in 12 min, Wavelength: 254 nm) to afford (1R,2S,3R,5R)-3-{4-amino-5-[1-(benzenesulfonyl)pyrazol-3-yl]-2-chloropyrrolo[2,3-d]pyrimidin-7-yl}-5-[1-(2-phenylethyl)piperidin-4-yl]cyclopentane-1,2-diol 519 (12.7 mg, 20%) as an off-white solid. LCMS (ZORBAX SB-Aq, 50*4.6 mm, 1.8 μm, Mobile Phase A: Water/0.02% TFA, Mobile Phase B: Acetonitrile, 5%-95% B-3 min, 1.50 L/min): T_{R}=1.61 min; ES m/z [M+H]⁺: 662, 664 ¹H NMR (400 MHZ, DMSO-d₆) δ8.71 (br, s, 1H), 8.55 (d, J=2.9 Hz, 1H), 8.11 (s, 1H), 8.03-7.91 (m, 2H), 7.89 (br, s, 1H), 7.84-7.78 (m, 1H), 7.74-7.65 (m, 2H), 7.30-7.24 (m, 2H), 7.24-7.14 (m, 3H), 7.12-7.08 (m, 1H), 4.82 (d, J=6.7 Hz, 1H), 4.79-4.69 (m, 1H), 4.61 (d, J=4.8 Hz, 1H), 4.09-3.99 (m, 1H), 3.82-3.72 (m, 1H), 3.03-2.87 (m, 2H), 2.76-2.69 (m, 2H), 2.60-2.59 (m, 2H), 2.10-1.98 (m, 1H), 1.98-1.79 (m, 3H), 1.74-1.63 (m, 1H), 1.62-1.54 (m, 1H), 1.53-1.40 (m, 1H), 1.35-1.11 (m, 3H).

### (1R,2S,3R,5R)-3-{4-amino-5-[1-(benzenesulfonyl)pyrazol-3-yl]-2-chloropyrrolo[2,3-d]pyrimidin-7-yl)-5-[1-(cyclopropylmethyl)piperidin-4-yl]cyclopentane-1,2-diol (520)

### (1R,2S,3R,5R)-3-(4-Amino-5-bromo-2-chloro-7H-pyrrolo[2,3-d]pyrimidin-7-yl)-5-(piperidin-4-yl)cyclopentane-1,2-diol (120b)

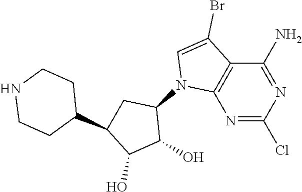 [SMILES: Nc1nc(Cl)nc2c1c(Br)cn2[C@@H]1C[C@H](C2CCNCC2)[C@@H](O)[C@H]1O]

Tert-butyl 4-((3aR,4R,6R,6aS)-6-(4-amino-5-bromo-2-chloro-7H-pyrrolo[2,3-d]pyrimidin-7-yl)-2,2-dimethyltetrahydro-4H-cyclopenta[d][1,3]dioxol-4-yl)piperidine-1-carboxylate (250 mg, 0.44 mmol, 1.o eq) in 4M HCl in MeOH (5.0 mL) was stirred for 20 min at 60° C. The resulting mixture was concentrated under reduced pressure to afford (1R,2S,3R,5R)-3-(4-amino-5-bromo-2-chloro-7H-pyrrolo[2,3-d]pyrimidin-7-yl)-5-(piperidin-4-yl)cyclopentane-1,2-diol hydrochloride 120b (250 mg, crude) as light yellow solid. LCMS (conditions: L-column3 ODS, 50*3.0 mm, 3.0 μm, Mobile Phase Water/5 mM NH₄HCO₃+0.05% Ammonia, Mobile Phase B: Acetonitrile/5% water, 2%-100% B-3 min, 1.50 L/min): T_{R}=0.69 min; ES m/z [M+H]⁺: 430, 432.

### (1R,2S,3R,5R)-3-(4-amino-5-bromo-2-chloro-7H-pyrrolo[2,3-d]pyrimidin-7-yl)-5-(1-(cyclopropylmethyl)piperidin-4-yl)cyclopentane-1,2-diol (121b)

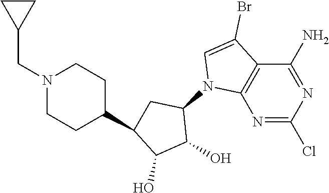 [SMILES: Nc1nc(Cl)nc2c1c(Br)cn2[C@@H]1C[C@H](C2CCN(CC3CC3)CC2)[C@@H](O)[C@H]1O]

Into a 40 mL vial was placed a solution of (1R,2S,3R,5R)-3-(4-amino-5-bromo-2-chloro-7H-pyrrolo[2,3-d]pyrimidin-7-yl)-5-(piperidin-4-yl)cyclopentane-1,2-diol 120b (0.25 g, 0.6 mmol, 1.0 eq) in DCM (4.0 mL). Cyclopropanecarbaldehyde (61.0 mg, 0.9 mmol, 1.5 eq), DIEA (0.15 g, 1.2 mmol, 2.0 eq), then NaBH (OAc) 3 (0.25 g, 1.2 mmol, 2.0 eq) were added. The mixture was stirred for 2h then concentrated under reduced pressure. The residue was dissolved in MeOH (2.0 mL) and purified by Prep-HPLC (Column: Welch Xtimate C18 ExRS, 250 mm, 10 μm; Mobile Phase A: Water (0.05% NH₃·H₂O), Mobile Phase B: ACN; Flow rate: 90 mL/min; Gradient: 20% B to 60% B over 10 min; Wavelength: 254 nm) to afford (1R,2S,3R,5R)-3-(4-amino-5-bromo-2-chloro-7H-pyrrolo[2,3-d]pyrimidin-7-yl)-5-(1-(cyclopropylmethyl)piperidin-4-yl)cyclopentane-1,2-diol 121b (100 mg, 35%) as an off-white solid. LCMS (conditions: L-column3 ODS, 50*3.0 mm, 3.0 μm, Mobile Phase Water/5 mM NH₄HCO₃+0.05% Ammonia, Mobile Phase B: Acetonitrile/5% water, 2%-100% B-3 min, 1.50 L/min): T_{R}=1.78 min; ES m/z [M+H]⁺: 484, 486.

### (1R,2S,3R,5R)-3-{4-Amino-5-[1-(benzenesulfonyl)pyrazol-3-yl]-2-chloropyrrolo[2,3-d]pyrimidin-7-yl}-5-[1-(cyclopropylmethyl)piperidin-4-yl]cyclopentane-1,2-diol (520)

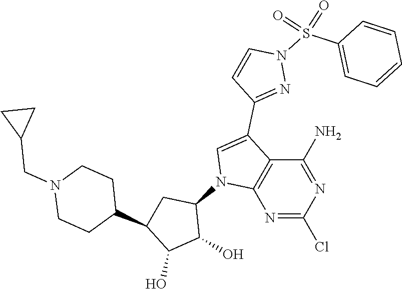 [SMILES: Nc1nc(Cl)nc2c1c(-c1ccn(S(=O)(=O)c3ccccc3)n1)cn2[C@@H]1C[C@H](C2CCN(CC3CC3)CC2)[C@@H](O)[C@H]1O]

Into an 8 mL vial was placed a solution of (1R,2S,3R,5R)-3-{4-amino-5-bromo-2-chloropyrrolo[2,3-d]pyrimidin-7-yl}-5-[1-(cyclopropylmethyl)piperidin-4-yl]cyclopentane-1,2-diol 121b (20.0 mg, 0.041 mmol, 1.0 eq) in 1,4-dioxane (0.5 mL) and H₂O (0.5 mL), and 1-(benzenesulfonyl)pyrazol-3-ylboronic acid (13.52 mg, 0.053 mmol, 1.3 eq), K₃PO₄ (27 mg, 0.123 mmol, 3.0 eq) and Pd (dtbpf) Cl₂ (6.1 mg, 0.009 mmol) were added. The mixture was stirred for 1 h at 80° C. under N₂ atmosphere. The resulting mixture was filtered. The filtrate was purified by Prep-HPLC (Column: Welch Xtimate C18 ExRS, 250 mm, 10 μm; Mobile Phase A: Water (0.05% NH₃·H₂O), Mobile Phase B: ACN; Flow rate: 90 mL/min; Gradient: 15% B to 60% B over 10 min, 90% B; Wavelength: 254 nm) to afford (1R,2S,3R,5R)-3-{4-amino-5-[1-(benzenesulfonyl)pyrazol-3-yl]-2-chloropyrrolo[2,3-d]pyrimidin-7-yl}-5-[1-(cyclopropylmethyl)piperidin-4-yl]cyclopentane-1,2-diol 520 (7.7 mg, 31%) as an off-white solid. LCMS (ZORBAX SB-Aq, 50*4.6 mm, 1.8 μm, Mobile Phase A: Water/0.02% TFA, Mobile Phase B: Acetonitrile, 5%-(20%-50%)-95% B-5 min, 1.50 L/min): T_{R}=2.79 min; ES m/z [M+H]⁺: 612, 614 ¹H NMR (400 MHZ, DMSO-d₆) δ8.71 (br, s, 1H), 8.55 (d, J=2.9 Hz, 1H), 8.11 (s, 1H), 7.99-7.93 (m, 2H), 7.89 (br, s, 1H), 7.83-7.76 (m, 1H), 7.74-7.65 (m, 2H), 7.10 (d, J=2.9 Hz, 1H), 4.93-4.49 (m, 3H), 4.10-3.97 (m, 1H), 3.79-3.71 (m, 1H), 3.02-2.90 (m, 2H), 2.13 (d, J=6.5 Hz, 2H), 2.08-1.96 (m, 1H), 1.90-1.77 (m, 3H), 1.73-1.62 (m, 1H), 1.60-1.52 (m, 1H), 1.51-1.38 (m, 1H), 1.33-1.14 (m, 3H), 0.87-0.72 (m, 1H), 0.49-0.37 (m, 2H), 0.10-0.02 (m, 2H).

### Synthesis of (1R,2S,3R,5R)-3-[4-amino-5-(4-benzyl-1,3-thiazol-2-yl)-2-chloropyrrolo[2,3-d]pyrimidin-7-yl]-5-[1-(oxolan-3-ylmethyl)piperidin-4-yl]cyclopentane-1,2-diol (521)

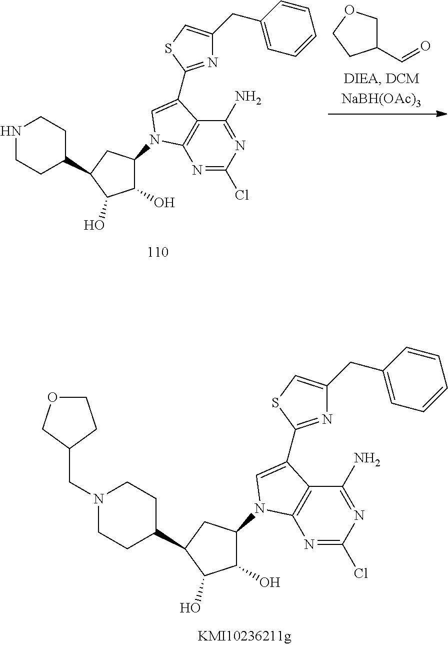

To a stirred mixture of (1R,2S,3R,5R)-3-[4-amino-5-(4-benzyl-1,3-thiazol-2-yl)-2-chloropyrrolo[2,3-d]pyrimidin-7-yl]-5-(piperidin-4-yl)cyclopentane-1,2-diol (150.0 mg, 0.2 mmol, 1.0 equiv), NaBH (OAc) 3 (121.0 mg, 0.5 mmol, 2.0 equiv) and DIEA (110.7 mg, 0.8 mmol, 3.0 equiv) in DCM (1.5 mL) was added oxolane-3-carbaldehyde (57.2 mg, 0.5 mmol, 2.0 equiv) dropwise at room temperature. The resulting mixture was stirred for 2 h at room temperature. The reaction was monitored by LCMS. The resulting mixture was concentrated under reduced pressure. The residue was dissolved in MeOH (5 mL) and was purified by Prep-HPLC with the following conditions (Column: Xselect Peptide CSH C18 19*150 mm 5 μm, 1; Mobile Phase A: Water (0.1% HCl), Mobile Phase B: ACN; Flow rate: 20 mL/min; Gradient: 33% B to 8% B in 8 min, 27% B; Wave Length: 254 nm & 220 nm nm) to afford (1R,2S,3R,5R)-3-[4-amino-5-(4-benzyl-1,3-thiazol-2-yl)-2-chloropyrrolo[2,3-d]pyrimidin-7-yl]-5-[1-(oxolan-3-ylmethyl)piperidin-4-yl]cyclopentane-1,2-diol 521 (80.4 mg, 46%) as a greenish solid. LCMS (conditions ZORBAX SB-Aq, 50*4.6 mm, 1.8 μm, Mobile Phase A: Water/0.05% TFA, Mobile Phase B: Acetonitrile/0.05% TFA, 5%-95% B-3 min): T_{R}=1.69 min; [M+H]⁺: 609, 611. ¹H NMR (400 MHZ, DMSO-d₆+D₂O) δ 8.17 (s, 1H), 7.37-7.17 (m, 6H), 4.80-4.69 (m, 1H), 4.24-4.13 (m, 1H), 4.06 (s, 2H), 3.89-3.78 (m, 2H), 3.76-3.69 (m, 2H), 3.63 (q, J=7.7 Hz, 1H), 3.59-3.37 (m, 2H), 3.21-3.02 (m, 2H), 2.96-2.79 (m, 2H), 2.73-2.61 (m, 1H), 2.15-2.00 (m, 3H), 1.82-1.70 (m, 1H), 1.73-1.50 (m, 6H).

### Synthesis of (1R,2S,3R,5R)-3-(4-amino-5-(4-benzylthiazol-2-yl)-2-chloro-7H-pyrrolo[2,3-d]pyrimidin-7-yl)-5-(1-(((R)-tetrahydrofuran-3-yl)methyl)piperidin-4-yl)cyclopentane-1,2-diol (521R, assumed structure) and (1R,2S,3R,5R)-3-(4-amino-5-(4-benzylthiazol-2-yl)-2-chloro-7H-pyrrolo[2,3-d]pyrimidin-7-yl)-5-(1-(((S)-tetrahydrofuran-3-yl)methyl)piperidin-4-yl)cyclopentane-1,2-diol (521S. assumed structure)

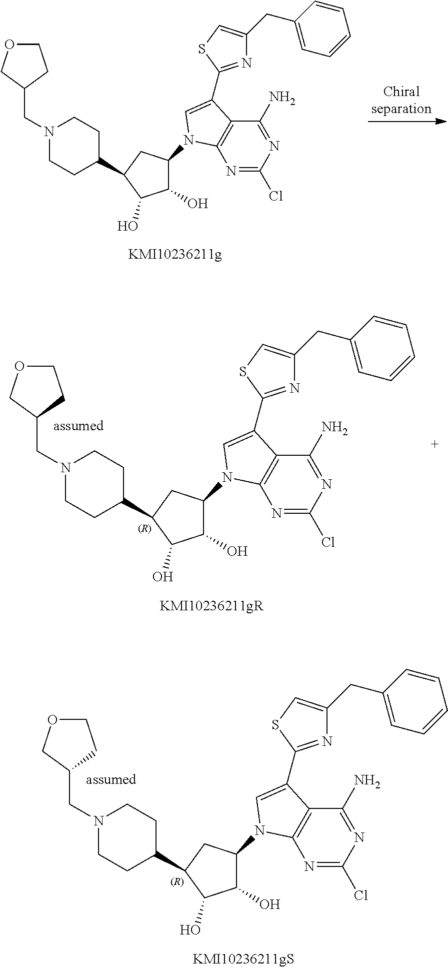

(1R,2S,3R,5R)-3-(4-amino-5-(4-benzylthiazol-2-yl)-2-chloro-7H-pyrrolo[2,3-d]pyrimidin-7-yl)-5-(1-((tetrahydrofuran-3-yl)methyl)piperidin-4-yl)cyclopentane-1,2-diol 521 (74.5 mg, 0.1 mmol, 1.0 equiv) was separated with Chiral HPLC (Column: CHIRALPAK IH, 3*25 cm, 5 μm; Mobile Phase A: Hex (0.1% 2M NH₃-MeOH)--HPLC, Mobile Phase B: EtOH: DCM=1:1--HPLC; Flow rate: 35 mL/min; Gradient: 20% B to 20% B in 20 min; Wave Length: 254/220 nm, Sample Solvent: MeOH--HPLC; Injection Volume: 0.8 mL) to afford (1R,2S,3R,5R)-3-(4-amino-5-(4-benzylthiazol-2-yl)-2-chloro-7H-pyrrolo[2,3-d]pyrimidin-7-yl)-5-(1-(((R)-tetrahydrofuran-3-yl)methyl)piperidin-4-yl)cyclopentane-1,2-diol 521R (assumed structure)(19.0 mg, 25%) as a white solid. LCMS (conditions SB-Aq, 50*3.0 mm, 1.8 μm Mobile Phase A: 0.05% TFA in H₂O Mobile Phase B: 0.05% TFA in Acetonitrile, 5%-95% B-3 min (+).lcm, 1.50 L/min, ES, m/z,): T_{R}=1.443 min; [M+H]⁺: 609, 611. ¹H NMR (400 MHZ, DMSO-d₆+D₂O) δ 8.13 (s, 1H), 7.37-7.18 (m, 6H), 4.79-4.68 (m, 1H), 4.24-4.13 (m, 1H), 4.06 (s, 2H), 3.88-3.70 (m, 3H), 3.64 (q, J=7.7 Hz, 1H), 3.58-3.50 (m, 2H), 3.45-3.36 (m, 1H), 3.22-3.04 (m, 2H), 2.94-2.79 (m, 2H), 2.68-2.58 (m, 1H), 2.15-2.01 (m, 3H), 1.84-1.77 (m, 1H), 1.74-1.45 (m, 6H).

(1R,2S,3R,5R)-3-(4-amino-5-(4-benzylthiazol-2-yl)-2-chloro-7H-pyrrolo[2,3-d]pyrimidin-7-yl)-5-(1-(((R)-tetrahydrofuran-3-yl)methyl)piperidin-4-yl)cyclopentane-1,2-diol 521S (assumed structure) (15.5 mg, 20%) as a white solid. LCMS (conditions SB-Aq, 50*3.0 mm, 1.8 μm Mobile Phase A: 0.05% TFA in H₂O Mobile Phase B: 0.05% TFA in Acetonitrile, 5%-95% B-3 min (+).lcm, 1.50 L/min, ES, m/z,): T_{R}=1.441 min; [M+H]⁺: 609, 610. ¹H NMR (400 MHZ, DMSO-d₆+D₂O) δ 8.13 (s, 1H), 7.48-7.12 (m, 6H), 4.79-4.68 (m, 1H), 4.22-4.13 (m, 1H), 4.06 (s, 2H), 3.97-3.69 (m, 3H), 3.69-3.59 (m, 1H), 3.46-3.32 (m, 2H), 3.29-3.00 (m, 3H), 3.01-2.77 (m, 2H), 2.74-2.58 (m, 1H), 2.11-2.03 (m, 3H), 1.91-1.73 (m, 1H), 1.73-1.38 (m, 6H).

### Synthesis of (1R,2S,3R,5R)-3-(5-(1-benzyl-1H-pyrazol-3-yl)-2-chloro-7H-pyrrolo[2,3-d]pyrimidin-7-yl)-5-(piperidin-4-yl)cyclopentane-1,2-diol (522)

### (1R,2S,3R,5R)-3-(5-(1-Benzyl-1H-pyrazol-3-yl)-2-chloro-7H-pyrrolo[2,3-d]pyrimidin-7-yl)-5-(piperidin-4-yl)cyclopentane-1,2-diol (522)

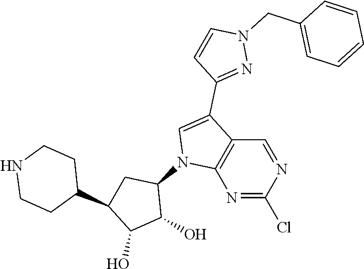 [SMILES: O[C@@H]1[C@H](O)[C@@H](C2CCNCC2)C[C@H]1n1cc(-c2ccn(Cc3ccccc3)n2)c2cnc(Cl)nc21]

Into a 40 mL vial were added tert-butyl 4-((3aR,4R,6R,6aS)-6-(5-(1-benzyl-1H-pyrazol-3-yl)-2-chloro-7H-pyrrolo[2,3-d]pyrimidin-7-yl)-2,2-dimethyltetrahydro-4H-cyclopenta[d][1,3]dioxol-4-yl)piperidine-1-carboxylate 68b (20 mg, 0.03 mmol, 1.0 eq) and 4M HCl in MeOH (2.0 mL). The mixture was stirred at 40° C. for 20 min, then concentrated under reduced pressure. The crude product was dissolved into MeOH (2.0 mL) and basified to pH>8 with NH₃·H₂O, before being purified by reverse phase flash chromatography, using the following conditions (Column: Welch Ultimate AQ-C18, 50*250 mm*10 μm; Mobile Phase A: Water (10 mmol/L NH₄HCO₃+0.1% NH₃·H₂O), Mobile Phase B: ACN; Flow rate: 80 ml/min; Gradient: 5% B to 65% B over 10 min, Wavelength: 220 nm) to afford (1R,2S,3R,5R)-3-(5-(1-benzyl-1H-pyrazol-3-yl)-2-chloro-7H-pyrrolo[2,3-d]pyrimidin-7-yl)-5-(piperidin-4-yl)cyclopentane-1,2-diol 522 (7.8 mg, 50%) as a white solid. LCMS (Xbridge BEH C18, 50*3.0 mm, 2.5 μm, Mobile Phase A: Water/0.05% ammonia water, Mobile Phase B: MeOH, 5%-95% B-3.0 min, 1.20 L/min): T_{R}=1.39 min; ES m/z [M+H]⁺: 493, 495. 1H NMR (400 MHZ, DMSO-d₆) δ 9.23 (s, 1H), 8.19 (s, 1H), 7.89 (d, J=2.3 Hz, 1H), 7.41-7.22 (m, 5H), 6.73 (d, J=2.3 Hz, 1H), 5.39 (s, 2H), 4.89 (q, J=9.5 Hz, 1H), 4.14 (dd, J=9.4, 6.2 Hz, 1H), 3.83-3.76 (m, 1H), 2.98 (t, J=10.7 Hz, 2H), 2.49-2.44 (m, 2H), 2.14-2.03 (m, 1H), 1.87-1.79 (m, 1H), 1.74-1.54 (m, 3H), 1.46-1.39 (m, 1H), 1.24-1.09 (m, 2H).

### Synthesis of (1R,2S,3R,5R)-3-(4-amino-5-(4-benzylthiazol-2-yl)-2-chloro-7H-pyrrolo[2,3-d]pyrimidin-7-yl)-5-(1-(cyclopentylmethyl)piperidin-4-yl)cyclopentane-1,2-diol (523)

### (1R,2S,3R,5R)-3-(4-Amino-5-(4-benzylthiazol-2-yl)-2-chloro-7H-pyrrolo[2,3-d]pyrimidin-7-yl)-5-(1-(cyclopentylmethyl)piperidin-4-yl)cyclopentane-1,2-diol (523)

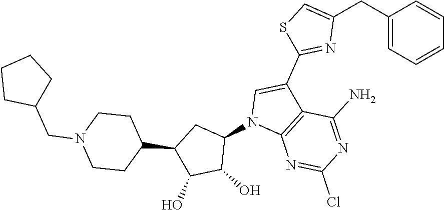 [SMILES: Nc1nc(Cl)nc2c1c(-c1nc(Cc3ccccc3)cs1)cn2[C@@H]1C[C@H](C2CCN(CC3CCCC3)CC2)[C@@H](O)[C@H]1O]

Into an 8 mL vial was added a solution of (1R,2S,3R,5R)-3-[4-amino-5-(4-benzyl-1,3-thiazol-2-yl)-2-chloropyrrolo[2,3-d]pyrimidin-7-yl]-5-(piperidin-4-yl)cyclopentane-1,2-diol 110b (50 mg, 0.1 mmol, 1.0 eq) in DCM (1.0 mL), then cyclopentanecarboxaldehyde (12 mg, 0.13 mmol, 1.3 eq), DIEA (25 mg, 0.2 mmol, 2.0 eq) and NaBH (OAc) 3 (41 mg, 0.2 mmol, 2.0 eq) were added. The resulting mixture was stirred for 1 h, then concentrated under reduced pressure. The residue was purified by Prep-HPLC with the following conditions (Column: Welch Utimate AQ-C18, 50*250 mm*10 μm; Mobile Phase A: Water (10 mmol/L NH₄HCO₃+0.1% NH₃: H₂O), Mobile Phase B: ACN; Flow rate: 80 mL/min; Gradient: 5% B to 65% B over 10 min, Wavelength: 220 nm) to afford (1R,2S,3R,5R)-3-[4-amino-5-(4-benzyl-1,3-thiazol-2-yl)-2-chloropyrrolo[2,3-d]pyrimidin-7-yl]-5-[1-(cyclopentylmethyl)piperidin-4-yl]cyclopentane-1,2-diol 523 (15 mg, 26%) as a white solid. LCMS (conditions: Cortecs C18+, 50*3.0 mm, 2.7 μm, Mobile Phase A: Water/0.02% TFA, Mobile Phase B: Acetonitrile, 5%-95% B-3 min, 1.50 L/min): T_{R}=1.23 min; ES m/z [M+H]⁺: 607, 609.1H NMR (400 MHZ, DMSO-d₆) δ 9.43 (br, s, 1H), 8.16 (s, 1H), 7.82 (br, s, 1H), 7.37-7.26 (m, 4H), 7.26-7.18 (m, 2H), 4.81 (d, J=6.9 Hz, 1H), 4.80-4.69 (m, 1H), 4.57 (d, J=4.7 Hz, 1H), 4.16 (dd, J=9.3, 6.6 Hz, 1H), 4.07 (s, 2H), 3.80-3.71 (m, 1H), 2.92-2.82 (m, 2H), 2.15 (s, 1H), 2.13 (s, 1H), 2.10-1.96 (m, 2H), 1.86-1.75 (m, 3H), 1.74-1.60 (m, 3H), 1.60-1.39 (m, 6H), 1.24 (s, 2H), 1.21-1.09 (m, 3H).

### Synthesis of (15,2R,3R,5R)-3-(1-((1H-pyrazol-5-yl)methyl)piperidin-4-yl)-5-(4-amino-5-(4-benzylthiazol-2-yl)-2-chloro-7H-pyrrolo[2,3-d]pyrimidin-7-yl)cyclopentane-1,2-diol (524)

### (1S,2R,3R,5R)-3-(1-((1H-Pyrazol-5-yl)methyl)piperidin-4-yl)-5-(4-amino-5-(4-benzylthiazol-2-yl)-2-chloro-7H-pyrrolo[2,3-d]pyrimidin-7-yl)cyclopentane-12-diol (524)

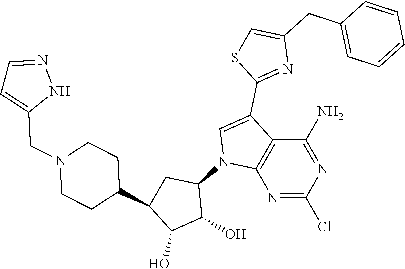 [SMILES: Nc1nc(Cl)nc2c1c(-c1nc(Cc3ccccc3)cs1)cn2[C@@H]1C[C@H](C2CCN(Cc3ccn[nH]3)CC2)[C@@H](O)[C@H]1O]

Into an 8 mL vial was added a solution of (1R,2S,3R,5R)-3-[4-amino-5-(4-benzyl-1,3-thiazol-2-yl)-2-chloropyrrolo[2,3-d]pyrimidin-7-yl]-5-(piperidin-4-yl)cyclopentane-1,2-diol 110b (50 mg, 0.1 mmol, 1.0 eq) in DCM (1.0 mL), followed by 2H-pyrazole-3-carbaldehyde (28 mg, 0.3 mmol, 3.0 eq), DIEA (25 mg, 0.2 mmol, 2.0 eq) and NaBH (AcO) 3 (41 mg, 0.2 mmol, 2.0 eq). The resulting mixture was stirred for 3 h, then concentrated under reduced pressure. The crude product was purified by Prep-HPLC with the following conditions (Column: Welch Ultimate XB-C18, 50*250 mm, 10 μm; Mobile Phase A: 0.1% HCl, Mobile Phase B: ACN; Flow rate: 90 mL/min; Gradient: 5% B-5% B-3 min, 10% B-45% B-12 min) to afford (1R,2S,3R,5R)-3-[4-amino-5-(4-benzyl-1,3-thiazol-2-yl)-2-chloropyrrolo[2,3-d]pyrimidin-7-yl]-5-[1-(2H-pyrazol-3-ylmethyl)piperidin-4-yl]cyclopentane-1,2-diol 2HCl salt 524 (26.5 mg, 46%) as a white solid. LCMS (conditions: Cortecs C18+, 50*3.0 mm, 2.7 μm, Mobile Phase A: Water/0.02% TFA, Mobile Phase B: Acetonitrile, 5%-95% B-3 min, 1.50 L/min): T_{R}=1.08 min; ES m/z [M+H]⁺: 605, 607.1H NMR (400 MHZ, DMSO-do+D₂O) δ 8.08 (s, 1H), 7.80 (d, J=2.4 Hz, 1H), 7.35-7.16 (m, 6H), 6.51 (d, J=2.4 Hz, 1H), 4.80-4.65 (m, 1H), 4.26 (s, 2H), 4.15 (dd, J=9.2, 6.3 Hz, 1H), 4.04 (s, 2H), 3.81-3.74 (m, 1H), 3.49-2.78 (m, 4H), 2.12-1.99 (m, 2H), 1.85-1.77 (m, 1H), 1.71-1.38 (m, 5H).

### Synthesis of (1R,2S,3R,5R)-3-(4-amino-5-(4-benzylthiazol-2-yl)-2-chloro-7H-pyrrolo[2,3-d]pyrimidin-7-yl)-5-(1-(furan-2-ylmethyl)piperidin-4-yl)cyclopentane-1,2-diol (525)

### (1R,2S,3R,5R)-3-(4-amino-5-(4-benzylthiazol-2-yl)-2-chloro-7H-pyrrolo[2,3-d]pyrimidin-7-yl)-5-(1-(furan-2-ylmethyl)piperidin-4-yl)cyclopentane-1,2-diol (525)

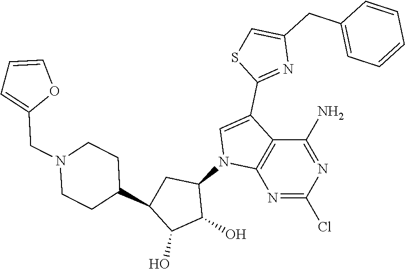 [SMILES: Nc1nc(Cl)nc2c1c(-c1nc(Cc3ccccc3)cs1)cn2[C@@H]1C[C@H](C2CCN(Cc3ccco3)CC2)[C@@H](O)[C@H]1O]

Into an 8 mL vial was added a solution of (1R,2S,3R,5R)-3-[4-amino-5-(4-benzyl-1,3-thiazol-2-yl)-2-chloropyrrolo[2,3-d]pyrimidin-7-yl]-5-(piperidin-4-yl)cyclopentane-1,2-diol 110b (50 mg, 0.1 mmol, 1.0 equiv) in DCM (1.0 mL), followed by furan-2-carbaldehyde (13 mg, 0.13 mmol, 1.3 equiv), DIEA (25 mg, 0.2 mmol, 2.0 equiv) and NaBH (AcO) 3 (41 mg, 0.2 mmol, 2.0 equiv). The resulting mixture was stirred for 1 h, then concentrated under reduced pressure. The crude product was purified by Prep-HPLC with the following conditions (Column: Welch Utimate AQ-C18, 50*250 mm*10 μm; Mobile Phase A: Water (10 mmol/L NH₄HCO₃+0.1% NH₃: H₂O), Mobile Phase B: ACN; Flow rate: 80 mL/min; Gradient: 5% B to 65% B over 15 min; Wavelength: 220 nm) to afford (1R,2S,3R,5R)-3-[4-amino-5-(4-benzyl-1,3-thiazol-2-yl)-2-chloropyrrolo[2,3-d]pyrimidin-7-yl]-5-[1-(furan-2-ylmethyl)piperidin-4-yl]cyclopentane-1,2-diol 525 (12.5 mg, 22%) as a white solid. LCMS (conditions: Cortecs C18+, 50*3.0 mm, 2.7 μm, Mobile Phase A: Water/0.02% TFA, Mobile Phase B: Acetonitrile, 5%-95% B-3 min, 1.50 L/min): T_{R}=1.16 min; ES m/z [M+H]⁺: 605, 607. ¹H NMR (400 MHZ, DMSO-d₆) δ 9.42 (br, s, 1H), 8.15 (s, 1H), 7.81 (br, s, 1H), 7.56 (s, 1H), 7.38-7.26 (m, 4H), 7.26-7.18 (m, 2H), 6.42-6.36 (m, 1H), 6.26 (d, J=3.1 Hz, 1H), 4.81 (d, J=6.9 Hz, 1H), 4.74 (dd, J=10.6, 8.2 Hz, 1H), 4.58 (d, J=4.7 Hz, 1H), 4.15 (dd, J=9.4, 6.5 Hz, 1H), 4.07 (s, 2H), 3.79-3.71 (m, 1H), 3.46 (s, 2H), 2.88-2.77 (m, 2H), 2.07-1.99 (m, 1H), 1.99-1.87 (m, 2H), 1.87-1.78 (m, 1H), 1.70-1.61 (m, 1H), 1.61-1.50 (m, 2H), 1.32-1.14 (m, 3H).

### Synthesis of (1R,2S,3R,5R)-3-(4-amino-5-(4-benzylthiazol-2-yl)-2-chloro-7H-pyrrolo[2,3-d]pyrimidin-7-yl)-5-(1-(thiophen-2-ylmethyl)piperidin-4-yl)cyclopentane-1,2-diol (526)

### (1R,2S,3R,5R)-3-(4-Amino-5-(4-benzylthiazol-2-yl)-2-chloro-7H-pyrrolo[2,3-d]pyrimidin-7-yl)-5-(1-(thiophen-2-ylmethyl)piperidin-4-yl)cyclopentane-1,2-diol (526)

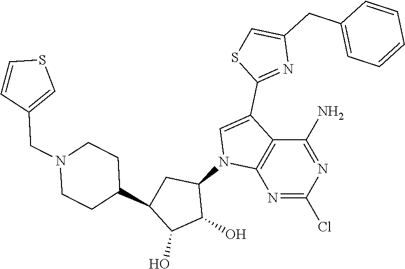 [SMILES: Nc1nc(Cl)nc2c1c(-c1nc(Cc3ccccc3)cs1)cn2[C@@H]1C[C@H](C2CCN(Cc3ccsc3)CC2)[C@@H](O)[C@H]1O]

Into an 8 mL vial was added a solution of (1R,2S,3R,5R)-3-[4-amino-5-(4-benzyl-1,3-thiazol-2-yl)-2-chloropyrrolo[2,3-d]pyrimidin-7-yl]-5-(piperidin-4-yl)cyclopentane-1,2-diol 110b (50 mg, 0.1 mmol, 1.0 eq) in DCM (1.0 mL), followed by thiophene-2-carboxaldehyde (14 mg, 0.13 mmol, 1.3 eq), DIEA (25 mg, 0.2 mmol, 2.0 eq) and NaBH (AcO) 3 (41 mg, 0.2 mmol, 2.0 eq). The resulting mixture was stirred for 1 h, then concentrated under reduced pressure. The crude product was purified by Prep-HPLC with the following conditions (Column: Welch Ultimate XB-C18, 50*250 mm, 10 μm; Mobile Phase A: 0.1% HCl, Mobile Phase B: ACN; Flow rate: 90 mL/min; Gradient: 5% B-5% B-3 min, 10% B-55% B-12 min) to afford (1R,2S,3R,5R)-3-[4-amino-5-(4-benzyl-1,3-thiazol-2-yl)-2-chloropyrrolo[2,3-d]pyrimidin-7-yl]-5-[1-(thiophen-2-ylmethyl)piperidin-4-yl]cyclopentane-1,2-diol 526 (13.7 mg, 23%) as a white solid. LCMS (conditions: Cortecs C18+, 50*3.0 mm, 2.7 μm, Mobile Phase A: Water/0.02% TFA, Mobile Phase B: Acetonitrile, 5%-95% B-3 min, 1.50 L/min): T_{R}=1.19 min; ES m/z [M+H]⁺: 621, 623. ¹H NMR (400 MHZ, DMSO-d₆+D₂O) δ 8.11 (s, 1H), 7.80-7.73 (m, 1H), 7.69-7.63 (m, 1H), 7.37-7.26 (m, 5H), 7.26-7.17 (m, 2H), 4.73 (dd, J=11.3, 8.8 Hz, 1H), 4.29 (s, 2H), 4.17 (dd, J=9.1, 6.4 Hz, 1H), 4.06 (s, 2H), 3.78 (dd, J=6.4, 3.9 Hz, 1H), 3.43-3.32 (m, 2H), 3.18-2.75 (m, 2H), 2.13-2.00 (m, 2H), 1.86-1.78 (m, 1H), 1.75-1.37 (m, 5H).

### Synthesis of (1R,2S,3R,5R)-3-(4-amino-5-(4-benzylthiazol-2-yl)-2-chloro-7H-pyrrolo[2,3-d]pyrimidin-7-yl)-5-(1-(pyridin-4-ylmethyl)piperidin-4-yl)cyclopentane-1,2-diol (527)

### (1R,2S,3R,5R)-3-(4-Amino-5-(4-benzylthiazol-2-yl)-2-chloro-7H-pyrrolo[2,3-d]pyrimidin-7-yl)-5-(1-(pyridin-4-ylmethyl)piperidin-4-yl)cyclopentane-1,2-diol (527)

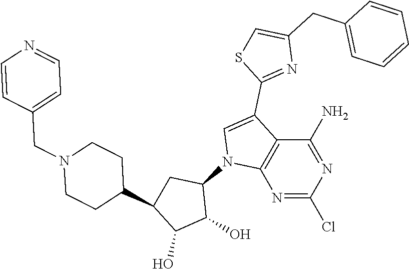 [SMILES: Nc1nc(Cl)nc2c1c(-c1nc(Cc3ccccc3)cs1)cn2[C@@H]1C[C@H](C2CCN(Cc3ccncc3)CC2)[C@@H](O)[C@H]1O]

Into an 8 mL vial was added a solution of (1R,2S,3R,5R)-3-[4-amino-5-(4-benzyl-1,3-thiazol-2-yl)-2-chloropyrrolo[2,3-d]pyrimidin-7-yl]-5-(piperidin-4-yl)cyclopentane-1,2-diol 110b (50 mg, 0.1 mmol, 1.0 eq) in DCM (0.8 mL), followed by 4-formylpyridine (14 mg, 0.13 mmol, 1.3 eq), DIEA (25 mg, 0.2 mmol, 2.0 eq) and NaBH (AcO) 3 (41 mg, 0.2 mmol, 2.0 eq). The resulting mixture was stirred for 1 h, then concentrated under reduced pressure. The crude product was purified by Prep-HPLC with the following conditions (Column: Welch Ultimate XB-C18, 50*250 mm, 10 μm; Mobile Phase A: 0.1% HCl, Mobile Phase B: ACN; Flow rate: 90 mL/min; Gradient: 5% B-5% B-3 min, 10% B-50% B-12 min) to afford (1R,2S,3R,5R)-3-[4-amino-5-(4-benzyl-1,3-thiazol-2-yl)-2-chloropyrrolo[2,3-d]pyrimidin-7-yl]-5-[1-(pyridin-4-ylmethyl)piperidin-4-yl]cyclopentane-1,2-diol 527 (23.2 mg, 40%) as a white solid. LCMS (conditions: Cortecs C18+, 50*3.0 mm, 2.7 μm, Mobile Phase A: Water/0.02% TFA, Mobile Phase B: Acetonitrile, 5%-95% B-3 min, 1.50 L/min): T_{R}=1.03 min; ES m/z [M+H]⁺: 616, 618. ¹H NMR (400 MHZ, DMSO-d₆+D₂O) δ 8.89 (d, J=6.0 Hz, 2H), 8.12 (s, 1H), 8.03 (d, J=5.6 Hz, 2H), 7.37-7.18 (m, 6H), 4.73 (q, J=9.0 Hz, 1H), 4.50 (s, 2H), 4.17 (dd, J=9.0, 6.4 Hz, 1H), 4.06 (s, 2H), 3.79 (dd, J=6.5, 3.9 Hz, 1H), 3.43-3.35 (m, 2H), 3.20-2.82 (m, 2H), 2.15-1.98 (m, 2H), 1.84-1.77 (m, 1H), 1.75-1.48 (m, 5H).

### (3aR,6R,6aR)-6-(hydroxymethyl)-2,2-dimethyltetrahydro-4H-cyclopenta[d][1,3]dioxol-4-one (528)

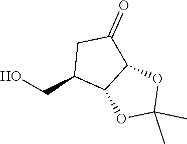

In a 1 L flask 2000 mg (12.84 mmol; 1.00 eq) of (3aR,6aR)-2,2-dimethyl-3a,6a-dihydro-4H-cyclopenta[d][1,3]dioxol-4-one were dissolved in 600 ml of MeOH. Afterwards, a catalytic amount of benzophenone (473 mg; 2.57 mmol; 0.20 eq) was added. The reaction mixture was flushed by N2 gas for 5 minutes and subsequently stirred and irradiated with a UV-lamp for 16 h at RT under reflux. The solvent was removed under reduced pressure and the remaining residue was purified by flash chromatography eluting with Cyclohexane/EtOAc (99:1% to 30:70%) to give title compound in form of a yellowish solid (1585 mg; 8.51 mmol; 66%). TLC: R_{f}=0.23 (Cyclohexane/EtOAc 1:1). ¹H-NMR: (DMSO-d₆, 400 MHZ) δ=4.92 (t, 1H, J=4.6 Hz), 4.63 (d, 1H, J=5.4 Hz), 4.10 (d, 1H, J=5.4 Hz), 3.61-3.54 (m, 1H), 3.46-3.39 (m, 1H), 2.62-2.55 (m, 1H), 2.42-2.33 (m, 1H), 2.05-1.96 (m, 1H), 1.31 (s, 3H), 1.26 (s, 3H) ppm.

### (3aR,6R,6aR)-6-(((tert-butyldimethylsilyl)oxy)methyl)-2,2-dimethyltetrahydro-4H cyclopenta[d][1,3]dioxol-4-one (529)

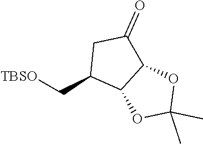 [SMILES: CC1(C)O[C@@H]2[C@@H](CO[Si](C)(C)C(C)(C)C)CC(=O)[C@@H]2O1]

1460 mg (7.84 mmol; 1.00 eq) of (3aR,6R,6aR)-6-(hydroxymethyl)-2,2-dimethyltetrahydro-4H-cyclopenta[d][1,3]dioxol-4-one, 1534 mg (23.52 mmol; 3.00 eq) of imidazole and 92 mg (0.78 mmol; 0.10 eq) of DMAP were dissolved in 40 mL of DMF. 1360 mg (9.41 mmol; 1.20 eq) of TBSCl were added. The reaction mixture was stirred for 18 h at RT and then quenched with 100 ml of a solution of a saturated NaHCO₃ solution and extracted with EtOAc (6×50 mL). The organic phases were combined, dried with MgSO₄, and then purified by flash chromatography eluting with Cyclohexane/EtOAc (99%/1% to 70%/30%) to give 2136 mg (7.11 mmol; 91%) of the title compound in form of a white solid. TLC: R_{f}=0.51 (Cyclohexane/EtOAc 5:1). ¹H-NMR: (DMSO-d₆, 400 MHZ) δ=4.65-4.60 (m, 1H), 4.09-4.04 (m, 1H), 3.77 (dd, 1H, J=9.8, 2.6 Hz), 3.58 (dd, 1H, J=9.8, 3.1 Hz), 2.66-2.59 (m, 1H), 2.46-2.39 (m, 1H), 1.95-1.91 (m, 1H), 1.32 (s, 3H), 1.25 (s, 3H), 0.81 (s, 9H), 0.02 (s, 3H), 0.00 (s, 3H) ppm.

### (3as,4s,6R,6aR)-6-(((tert-butyldimethylsilyl)oxy)methyl)-2,2-dimethyltetrahydro-4H cyclopenta[d][1,3]dioxol-4-ol (530)

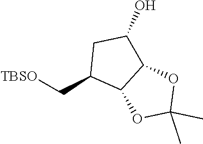 [SMILES: CC1(C)O[C@@H]2[C@@H](CO[Si](C)(C)C(C)(C)C)C[C@H](O)[C@@H]2O1]

In a 100 mL flask compound 2136 mg (7.11 mmol; 1.00 eq) of (3aR,6R,6aR)-6-(((tert-butyldimethylsilyl)oxy)methyl)-2,2-dimethyltetrahydro-4H-cyclopenta[d][1,3]dioxol-4-one were dissolved in 40 ml of MeOH. After cooling with an ice-bath to 0° C., 404 mg (10.67 mmol; 1.50 eq) of NaBH₄ were added portionwise. The reaction mixture was warmed to RT and thereby continuously stirred for 18 h. To purify the product flash chromatography was used eluting with Cyclohexane/EtOAc (99:1% to 75:25%) to the desired compound (2056 mg; 6.80 mmol; 96%) in form of a colorless liquid. TLC: R_{f}=0.45 (Cyclohexane/EtOAc 3:1). ¹H-NMR: (DMSO-d₆, 400 MHZ) δ=4.33 (d, 1H, J=6.9 Hz), 4.31-4.28 (m, 2H), 4.00-3.93 (m, 1H), 3.48-3.39 (m, 2H), 1.97-1.91 (m, 1H), 1.80-1.72 (m, 1H), 1.58-1.52 (m, 1H); 1.38 (s, 3H), 1.22 (s, 3H), 0.86 (s, 9H), 0.03 (s, 6H) ppm.

### 5-bromo-2,4-dichloro-7H-pyrrolo[2,3-d]pyrimidine (531)

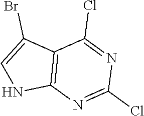 [SMILES: Clc1nc(Cl)c2c(Br)c[nH]c2n1]

1880 mg 2,4-dichloro-7H-pyrrolo[2,3-a]pyrimidine (10.00 mmol; 1.00 eq) were dissolved in 40 mL of DMF. 1958 mg NBS (11.00 mmol; 1.10 eq) were added and stirred for 16 h at RT. Afterwards the reaction mixture was quenched with 100 ml of a solution of saturated NaHCO₃ and then extracted with EtOAc (6×50 mL). The organic phases were combined, dried with MgSO₄, and purified by flash chromatography eluting with Cyclohexane/EtOAc (99:1% to 50:50%). 2194 mg (8.20 mmol; 82%) of the title compound were obtained in form of a white solid. TLC: R_{f}=0.57 (Cyclohexane/EtOAc 1:1). APCI-MS (+) m/z for C₆H₂BrCl₂N₃: calc: 264.88; found 265.8, 267.8 and 269.8. ¹H-NMR: (DMSO-d₆, 400 MHZ) δ=13.17 (s, 1H), 8.00 (s, 1H) ppm.

### 5-bromo-7-((3as,4R,6R,6aR)-6-(((tert-butyldimethylsilyl)oxy)methyl)-2,2-dimethyltetrahydro-4H-cyclopenta[d][1,3]dioxol-4-yl)-2,4-dichloro-7H-pyrrolo[2,3-d]pyrimidine (532)

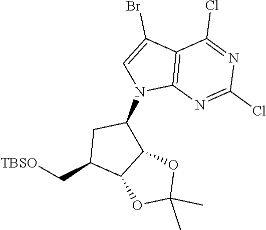 [SMILES: CC1(C)O[C@@H]2[C@@H](CO[Si](C)(C)C(C)(C)C)C[C@@H](n3cc(Br)c4c(Cl)nc(Cl)nc43)[C@@H]2O1]

1210 mg (4.00 mmol; 1.00 eq) of (3as,4,6R,6aR)-6-(((tert-butyldimethylsilyl)oxy)methyl)-2,2-dimethyltetrahydro-4H-cyclopenta[d][1,3]dioxol-4-ol were dissolved in 40 ml of Toluene and then cooled with an ice-bath to 0° C. With constant stirring, 2098 mg (8.00 mmol; 2.00 eq) of PPh₃ were added. After 5 min, 1842 mg (8.00 mmol; 2.00 eq) of DBAD were attached and 10 min later, 1067 mg (4.00 mmol; 1.00 eq) of 5-bromo-2,4-dichloro-7H-pyrrolo[2,3-d]pyrimidine were given to the reaction mixture. This was then heated to 90° C. under reflux for 15.75 h. Afterwards, the crude was purified by flash chromatography eluting with Petrolether/EtOAc (99:1% to 80:20%) to give 1161 mg (2.10 mmol; 53%) of the title compound in form of an orange oil. TLC: R_{f}=0.66 (Cyclohexane/EtOAc 3:1). APCI-MS (+) m/z for C₂₁H₃₀BrCl₂N₃O3Si: calc: 551.38; found 551.6. ¹H-NMR: (DMSO-d₆, 400 MHZ) δ=8.23 (s, 1H), 5.04-4.98 (m, 1H), 4.89-4.86 (m, 1H), 4.52-4.46 (m, 1H), 3.75-3.70 (m, 1H), 3.67-3.63 (m, 1H), 2.34-2.21 (m, 2H), 2.21-1.97 (m, 1H), 1.48 (s, 3H), 1.22 (s, 3H), 0.88 (s, 9H), 0.06 (s, 6H) ppm.

### 5-bromo-7-((3as,4R,6R,6aR)-6-(((tert-butyldimethylsilyl)oxy)methyl)-2,2-dimethyltetrahydro-4H-cyclopenta[d][1,3]dioxol-4-yl)-2-chloro-7H-pyrrolo[2,3-d]pyrimidin-4-amine (533)

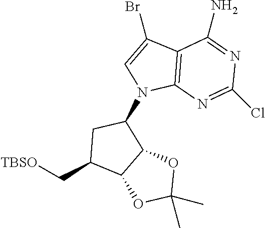 [SMILES: CC1(C)O[C@@H]2[C@@H](CO[Si](C)(C)C(C)(C)C)C[C@@H](n3cc(Br)c4c(N)nc(Cl)nc43)[C@@H]2O1]

1035 mg (1.88 mmol; 1.00 eq) of 5-bromo-7-((3as,4R,6R,6aR)-6-(((tert-butyldimethylsilyl)oxy)methyl)-2,2-dimethyltetrahydro-4H-cyclopenta[d][1,3]dioxol-4-yl)-2,4-dichloro-7H-pyrrolo[2,3-d]pyrimidine were dissolved in 10 ml of Dioxane. Subsequently, 40 mL of a 25% aqueous solution of NH₃ were added and then the reaction mixture was heated to 105° C. for 15.5 h with stirring. Then the solvent was removed under reduced pressure and the remaining residue was purified by flash chromatography eluting with DCM/MeOH (100:0% to 95:5%) to yield the title compound (640 mg; 1.20 mmol; 64%) in form of a colorless foam. TLC: R_{f}=0.94 (DCM/MeOH 10:1). APCI-MS (+) m/z for C₂₁H32BrClN4O3Si: calc.: 531.95; found 532.8. ¹H-NMR: (DMSO-d₆, 400 MHZ) δ=7.62 (s, 1H), 4.89-4.79 (m, 2H), 4.47-4.44 (m, 1H), 3.71-3.61 (m, 2H), 2.25-2.18 (m, 2H), 2.01-1.91 (m, 1H), 1.46 (s, 3H), 1.21 (s, 3H), 0.88 (s, 9H), 0.05 (s, 6H) ppm.

### ((3aR,4R,6R,6as)-6-(4-amino-5-bromo-2-chloro-7//pyrrolo[2,3-d]pyrimidin-7-yl)-2,2-dimethyltetrahydro-4H-cyclopenta[d][1,3]dioxol-4-yl)methanol (534)

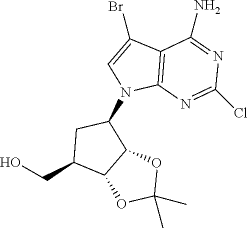 [SMILES: CC1(C)O[C@@H]2[C@@H](CO)C[C@@H](n3cc(Br)c4c(N)nc(Cl)nc43)[C@@H]2O1]

540 mg (1.20 mmol; 1.00 eq) 5-bromo-7-((3as,4R,6R,6aR)-6-(((tert-butyldimethylsilyl)oxy)methyl)-2,2-dimethyltetrahydro-4H-cyclopenta[d][1,3]dioxol-4-yl)-2-chloro-7H-pyrrolo[2,3-d]pyrimidin-4-amine were dissolved in 10.0 ml of THF and thereupon 1.80 mL of a 1 M solution of TBAF in THF (1.80 mmol; 1.50 eq) were added. The reaction mixture was then stirred at RT for 14.5 h. After removing the solvent under reduced pressure, the remaining residue was purified by flash chromatography eluting with DCM/MeOH (100:0% to 95:5%) to give 501 mg of the title compound (1.20 mmol; quantitative) in form of a white solid. TLC: R_{f}=0.77 (DCM/MeOH 10:1). APCI-MS (+) m/z for C₁₅H₁₈BrClN₄O₃: calc.: 417.69; found 416.4, 418.4 and 420.0. ¹H-NMR: (DMSO-d₆, 400 MHZ) δ=7.66 (s, 1H), 4.90-4.83 (m, 1H), 4.79-4.75 (m, 2H), 4.49-4.46 (m, 1H), 3.51-3.45 (m, 2H), 2.23-2.14 (m, 2H), 1.96-1.86 (m, 1H), 1.46 (s, 3H), 1.21 (s, 3H) ppm.

### (3aR,6R,6as)-6-(4-amino-5-bromo-2-chloro-7H-pyrrolo[2,3-d]pyrimidin-7-yl)-2,2-dimethyltetrahydro-4H-cyclopenta[d][1,3]dioxole-4-carbaldehyde (535)

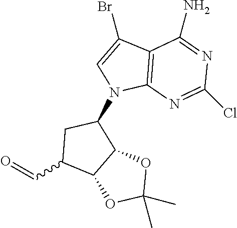 [SMILES: CC1(C)O[C@@H]2[C@H](O1)C(C=O)C[C@H]2n1cc(Br)c2c(N)nc(Cl)nc21]

250 mg (0.60 mmol; 1.0 eq) of ((3aR,4R,6R,6as)-6-(4-amino-5-bromo-2-chloro-7H-pyrrolo[2,3-d]pyrimidin-7-yl)-2,2-dimethyltetrahydro-4H-cyclopenta[d][1,3]dioxol-4-yl)methanol were dissolved in 30 ml of DCM. 509 mg (1.20 mmol; 2.0 eq) of Dess-Martin-periodinane were added at RT and the reaction solution was continuously stirred for 1.5. After addition of another 50 ml of DCM, the organic phase was washed with 50 ml of a saturated NaHCO₃ solution. The organic phase was dried over MgSO₄, filtered, and the solvent was removed under reduced pressure. Column chromatography on silica gel (Cyclohexane/EtOAc 99:1% to 0:100%) gave 160 mg (0.38 mmol; 63%) of the title compound in the form of a yellow foam and mixture of diastereomers. TLC: R_{f}=0.68 (Cyclohexane/EtOAc 1:1). ¹H-NMR: (DMSO-d₆, 400 MHZ) δ=9.71 (s, 1H), 7.46 (s, 1H), 5.31-5.25 (m, 1H), 4.89-4.79 (m, 2H), 3.59-3.47 (m, 1H), 2.39-2.20 (m, 1H), 2.02-1.90 (m, 1H), 1.37 (s, 3H), 1.25 (s, 3H) ppm.

### tert-butyl (3-((1//pyrazol-4-yl)amino)propyl)(4-fluorophenethyl)carbamate (536)

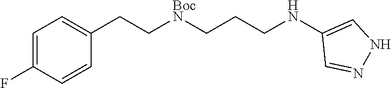 [SMILES: CC(C)(C)OC(=O)N(CCCNc1cn[nH]c1)CCc1ccc(F)cc1]

239 mg (2.88 mmol; 1.00 eq) of 1H-pyrazol-4-amine were dissolved in 30 ml of DCE and reacted at RT under N₂ atmosphere with 850 mg (2.88 mmol; 1.00 eq) of tert-butyl (4-fluorophenethyl)(3-oxopropyl)carbamate and 916 mg (4.32 mmol; 1.50 eq) of NaB (OAc) 3H. After 14 h of stirring the solvent was removed under reduced pressure and the resulting residue was adsorbed on silica gel. Column chromatography (DCM/MeOH 100:0% to 90:10%) gave 760 mg (2.10 mmol; 73%) of the title compound in the form of a colorless oil. TLC: R_{f}=0.46 (DCM/MeOH 10:1). APCI-MS (+) m/z for C₁₉H₂₇FN₄O₂: calc.: 362.45; found: 360.9, 361.9 and 363.1. ¹H-NMR: (DMSO-d₆, 400 MHZ) δ=12.17 (bs, 1H), 7.26-7.17 (m, 2H), 7.16-7.06 (m, 4H), 3.36-3.28 (m, 2H), 3.24-3.12 (m, 2H), 2.83 (t, J=7.0 Hz, 2H), 2.7-2.71 (m, 2H), 1.72-1.65 (m, 2H), 1.33 (s, 9H) ppm.

### tert-butyl (3-((((3aR,4R,6R,6as)-6-(4-amino-5-bromo-2-chloro-7H-pyrrolo[2,3-d]pyrimidin-7-yl)-2,2-dimethyltetrahydro-4H-cyclopenta[d][1,3]dioxol-4-yl)methyl)(1H-pyrazol-4-yl)amino)propyl)(4-fluorophenethyl)carbamate (537)

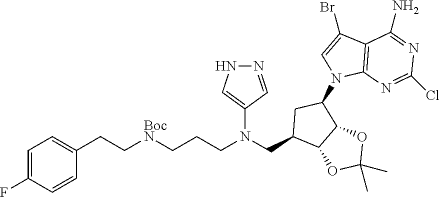 [SMILES: CC(C)(C)OC(=O)N(CCCN(C[C@H]1C[C@@H](n2cc(Br)c3c(N)nc(Cl)nc32)[C@@H]2OC(C)(C)O[C@H]12)c1cn[nH]c1)CCc1ccc(F)cc1]

290 mg (0.697 mmol; 1.00 eq) of (3aR,6R,6as)-6-(4-amino-5-bromo-2-chloro-7H-pyrrolo[2,3-d]pyrimidin-7-yl)-2,2-dimethyltetrahydro-4H-cyclopenta[d][1,3]dioxole-4-carbaldehyde were dissolved in 15 mL of DCE. 253 mg (0.697 mmol; 1.00 eq) of tert-butyl (3-((1//-pyrazol-4-yl)amino)propyl)(4-fluorophenethyl)carbamate and 222 mg (0.105 mmol; 1.50 eq) of NaB (OAc) 3H were added. The reaction mixture was stirred overnight and then the solvent was removed under reduced pressure. The resulting residue was adsorbed on silica gel and column chromatography (DCM/MeOH 100:0% to 95:5%) followed. 350 mg (0.459 mmol; 66%) of the title compound were received in the form of a colorless foam. TLC: R_{f}=0.58 (DCM/MeOH 10:1). APCI-MS (+) m/z for C₃₄H₄₃BrClFN₈O₄: calc.: 762.12; found: 760.5, 762.5 and 763.5.

### (1R,2S,3R,5R)-3-(4-amino-5-bromo-2-chloro-7H-pyrrolo[2,3-d]pyrimidin-7-yl)-5-(((3-((4-fluorophenethyl)amino)propyl)(1H-pyrazol-4-yl)amino)methyl)cyclopentane-1,2-diol (538)

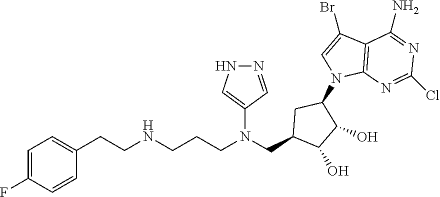 [SMILES: Nc1nc(Cl)nc2c1c(Br)cn2[C@@H]1C[C@H](CN(CCCNCCc2ccc(F)cc2)c2cn[nH]c2)[C@@H](O)[C@H]1O]

150 mg (0.196 mmol; 1.00 eq) of tert-butyl (3-((((3aR,4R,6R,6as)-6-(4-amino-5-bromo-2-chloro-7 H-pyrrolo[2,3-d]pyrimidin-7-yl)-2,2-dimethyltetrahydro-4H-cyclopenta[d][1,3]dioxol-4-yl)methyl)(1H-pyrazol-4-yl)amino)propyl)(4-fluorophenethyl)carbamate were dissolved in 6 mL TFA/H₂O (1:1) and stirred at RT for 18 h. The solvent was removed under reduced pressure and the residue was dissolved in methanol and purified by HPLC (MeCN/H₂O+0.1% TFA; 10/90% to 100/0% according to procedure A; t_{R}=13.39 min; UV-purity at 210, 230, 254, 242 and 275 nm >99%). 37 mg (0.043 mmol; 22%) of the title compound were obtained in the form of a colorless foam and as 2-fold TFA salt. APCI-MS (+) m/z for C₂₆H₃₁BrClFN₈O₂: calc.: 621.94; found: 620.4, 622.3 and 623.7. ¹H-NMR: (400 MHZ, DMSO-d₆) δ=8.47 (bs, 3H), 7.65 (s, 1H), 7.37 (bs, 1H), 7.33-7.27 (m, 2H), 7.20-7.15 (m, 2H), 4.94-4.87 (m, 1H), 4.28-4.22 (m, 1H), 3.86-3.81 (m, 1H), 3.30-3.21 (m, 1H), 3.19-3.07 (m, 4H), 3.02-2.92 (m, 3H), 2.90-2.85 (m, 2H), 2.59-2.54 (m, 1H), 2.00-1.91 (m, 1H), 1.80-1.70 (m, 3H) ppm.

### 7-((3as,4R,6R,6aR)-6-(((tert-butyldimethylsilyl)oxy)methyl)-2,2-dimethyltetrahydro-4H-cyclopenta[d][1,3]dioxol-4-yl)-2-chloro-5-(thiazol-2-yl)-7H-pyrrolo[2,3-d]pyrimidin-4-amine (539)

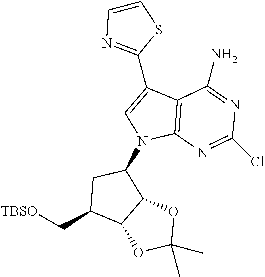 [SMILES: CC1(C)O[C@@H]2[C@@H](CO[Si](C)(C)C(C)(C)C)C[C@@H](n3cc(-c4nccs4)c4c(N)nc(Cl)nc43)[C@@H]2O1]

580 mg (1.00 mmol; 1.00 eq) of 7-((3as,4R,6R,6aR)-6-(((tert-butyldimethylsilyl)oxy)methyl)-2,2-dimethyltetrahydro-4H-cyclopenta[d][1,3]dioxol-4-yl)-2-chloro-5-iodo-7H-pyrrolo[2,3-d]pyrimidin-4-amine and 0.62 mL of Stannylthiazole (2.00 mmol; 2.00 eq) were dissolved in 30 ml of Toluene under inert gas atmosphere. Then the catalyst Pd (Ph₃)₂Cl₂ was added (70 mg; 0.110 mmol; 0.10 eq). The reaction mixture was heated under refluxation for 20 h. Afterwards the solvent was removed under reduced pressure and the crude was adsorbed on silica. Purification by flash chromatography eluting with Cyclohexane/EtOAc (99%/1% to 50%/50%) followed. The title compound (400 mg; 0.75 mmol; 75%) was obtained in form of colorless foam. APCI-MS (+) m/z for C₂₄H₃₄ClN₅O₃SSi: calc.: 535.18; found: 536.2 and 537.2. ¹H-NMR: (400 MHZ, DMSO-d₆) δ=9.56 (bs, 1H), 8.23 (s, 1H), 8.04 (bs, 1H), 7.86 (d, J=3.3 Hz, 1H), 7.69 (d, J=3.3 Hz, 1H), 4.99-4.91 (m, 2H), 4.56-4.50 (m, 1H), 3.76-3.67 (m, 2H), 2.32-2.23 (m, 2H), 2.12-2.00 (m, 1H), 1.50 (s, 3H), 1.25 (s, 3H), 0.91 (s, 9H), 0.08 (s, 6H) ppm.

### ((3aR,4R,6R,6as)-6-(4-amino-2-chloro-5-(thiazol-2-yl)-7H-pyrrolo[2,3-d]pyrimidin-7-yl)-2,2-dimethyltetrahydro-4H-cyclopenta[d][1,3]dioxol-4-yl)methanol (540)

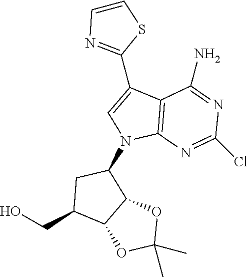 [SMILES: CC1(C)O[C@@H]2[C@@H](CO)C[C@@H](n3cc(-c4nccs4)c4c(N)nc(Cl)nc43)[C@@H]2O1]

400 mg (0.75 mmol; 1.00 eq) of 7-((3as,4R,6R,6aR)-6-(((tert-butyldimethylsilyl)oxy)methyl)-2,2-dimethyltetrahydro-4H-cyclopenta[d][1,3]dioxol-4-yl)-2-chloro-5-(thiazol-2-yl)-7H-pyrrolo[2,3-d]pyrimidin-4-amine were dissolved in 10 mL of THF and 1.10 ml of a 1M solution of TBAF in THF (1.10 mmol; 1.50 eq) were added. The reaction mixture was subsequently stirred at RT for 13 h. Then the solvent was removed under reduced pressure and The remaining residue was purified by flash chromatography eluting with DCM/MeOH (100:0% to 90:10%). 270 mg (0.64 mmol; 96%) of the title compound were isolated in form of a white solid. APCI-MS (+) m/z for C₁₈H₂₀ClN₅O₃S: calc.: 421.10; found: 422.0 and 423.1. ¹H-NMR: (400 MHZ, DMSO-d₆) δ=9.57 (bs, 1H), 8.31 (s, 1H), 8.04 (bs, 1H), 7.86 (d, J=3.3 Hz, 1H), 7.69 (d, J=3.3 Hz, 1H), 4.98-4.91 (m, 2H), 4.82-4.79 (m, 1H), 4.56-4.53 (m, 1H), 3.57-3.51 (m, 2H), 2.29-2.20 (m, 2H), 2.08-1.98 (m, 1H), 1.51 (s, 3H), 1.26 (s, 3H) ppm.

### (3aR,6R,6as)-6-(4-amino-2-chloro-5-(thiazol-2-yl)-7H-pyrrolo[2,3-d]pyrimidin-7-yl)-2,2-dimethyltetrahydro-4H-cyclopenta[d][1,3]dioxole-4-carbaldehyde (541)

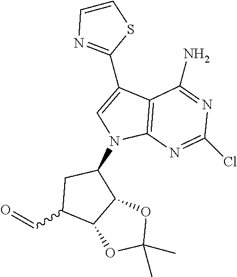 [SMILES: CC1(C)O[C@@H]2[C@H](O1)C(C=O)C[C@H]2n1cc(-c2nccs2)c2c(N)nc(Cl)nc21]

270 mg (0.64 mmol; 1.00 eq) of ((3aR,4R,6R,6as)-6-(4-amino-2-chloro-5-(thiazol-2-yl)-7H-pyrrolo[2,3-d]pyrimidin-7-yl)-2,2-dimethyltetrahydro-4H-cyclopenta[d][1,3]dioxol-4-yl)methanol were dissolved in 5 mL of DMSO. 204 mg (1.28 mmol; 2.00 eq) of pyridine sulfur trioxide and 0.18 mL (1.28 mmol; 2.00 eq) of Et₃N were added at RT. The reaction solution was continuously stirred for 3 h. The reaction was quenched with 50 mL of a saturated NaCl solution and extracted 5 times with 50 ml of EtOAc. The organic phase was dried over MgSO₄, filtered, and the solvent was removed under reduced pressure. Column chromatography on silica gel (Cyclohexane/EtOAc 99:1% to 0:100%) gave 120 mg (0.29 mmol; 45%) of the title compound in the form of a colorless foam and mixture of diastereomers. TLC: R_{f}=0.24 (Cyclohexane/EtOAc 1:1)

### tert-butyl (3-((((3aR,6R,6as)-6-(4-amino-2-chloro-5-(thiazol-2-yl)-7H-pyrrolo[2,3-d]pyrimidin-7-yl)-2,2-dimethyltetrahydro-4H-cyclopenta[d][1,3]dioxol-4-yl)methyl)(1//pyrazol-4-yl)amino)propyl)(4-fluorophenethyl)carbamate (542): 1

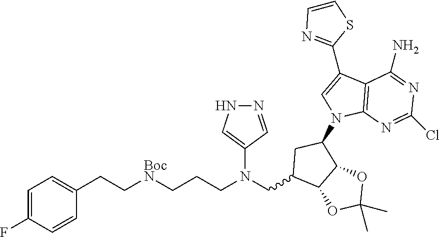 [SMILES: CC(C)(C)OC(=O)N(CCCN(CC1C[C@@H](n2cc(-c3nccs3)c3c(N)nc(Cl)nc32)[C@@H]2OC(C)(C)O[C@H]12)c1cn[nH]c1)CCc1ccc(F)cc1]

120 mg (0.290 mmol; 1.00 eq) of (3aR,6R,6as)-6-(4-amino-2-chloro-5-(thiazol-2-yl)-7H-pyrrolo[2,3-d]pyrimidin-7-yl)-2,2-dimethyltetrahydro-4H-cyclopenta[d][1,3]dioxole-4-carbaldehyde were dissolved in 15 mL of DCE. 105 mg (0.290 mmol; 1.00 eq) of tert-butyl (3-((1H-pyrazol-4-yl)amino)propyl)(4-fluorophenethyl)carbamate and 93 mg (0.440 mmol; 1.50 eq) of NaB (OAc) 3H were added. The reaction mixture was stirred overnight and then the solvent was removed under reduced pressure. The resulting residue was adsorbed on silica gel and column chromatography (DCM/MeOH 100:0% to 90:10%) followed. 130 mg (0.170 mmol; 59%) of the title compound were received in the form of a colorless foam. TLC: R_{f}=0.76 (DCM/MeOH 10:1). APCI-MS (+) m/z for C₃₇H45ClFN₉O₄S: calc.: 765.30; found: 766.4, 767.4 and 768.4.

### (1R,2S,3R)-3-(4-amino-2-chloro-5-(thiazol-2-yl)-7H-pyrrolo[2,3-d]pyrimidin-7-yl)-5-(((3-((4-fluorophenethyl)amino)propyl)(1H-pyrazol-4-yl)amino)methyl)cyclopentane-1,2-diol (543)

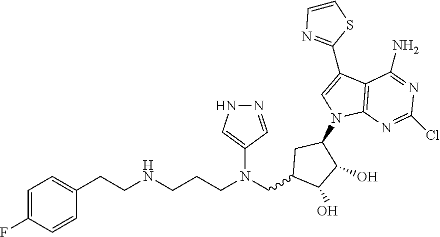 [SMILES: Nc1nc(Cl)nc2c1c(-c1nccs1)cn2[C@@H]1CC(CN(CCCNCCc2ccc(F)cc2)c2cn[nH]c2)[C@@H](O)[C@H]1O]

130 mg (0.169 mmol; 1.00 eq) of tert-butyl (3-((((3aR,6R,6as)-6-(4-amino-2-chloro-5-(thiazol-2-yl)-7H-pyrrolo[2,3-d]pyrimidin-7-yl)-2,2-dimethyltetrahydro-4H-cyclopenta[d][1,3]dioxol-4-yl)methyl)(1H-pyrazol-4-yl)amino)propyl)(4-fluorophenethyl)carbamate were dissolved in 8 mL TFA/H₂O (1:1) and stirred at RT for 24 h. The solvent was removed under reduced pressure and the residue was dissolved in methanol and purified by HPLC (MeCN/H₂O+0.1% TFA; 10/90% to 100/0% according to procedure B; t_{R}=10.60 and 10.86 min; UV-purity at 210, 230, 254, 242 and 275 nm >98%). 66 mg (0.077 mmol; 46%, stereoisomer 1) and 28 mg (0.033 mmol; 20%, stereoisomer 2) of the title compound were obtained in the form of a yellow foam (as 2-fold TFA salt). APCI-MS (+) m/z for C₂₉H₃₃ClFN₉O₂S: calc.: 625.22; found: 626.4, 627.5 and 628.4.

¹H-NMR for stereoisomer 1: (400 MHZ, DMSO-d₆) δ=9.52 (bs, 1H), 8.48 (bs, 2H), 8.24 (s, 1H), 7.96 (s, 1H), 7.84 (d, J=3.5 Hz, 1H), 7.64 (d, J=3.5 Hz, 1H), 7.46 (bs, 2H), 7.30-7.25 (m, 2H), 7.18-7.12 (m, 2H), 4.98-4.91 (m, 1H), 4.39 (dd, J=9.4, 3.4 Hz, 1H), 3.87-3.84 (m, 1H), 3.31 (bs, 1H), 3.23-3.10 (m, 4H), 3.04-2.94 (m, 3H), 2.90-2.82 (m, 2H), 2.65-2.56 (m, 1H), 2.03-1.95 (m, 1H), 1.84-1.75 (m, 3H) ppm.

¹H-NMR for stereoisomer 2: (400 MHZ, DMSO-d₆) δ=9.54 (bs, 1H), 8.47 (bs, 2H), 8.20 (s, 1H), 7.99 (s, 1H), 7.82 (d, J=3.5 Hz, 1H), 7.66 (d, J=3.3 Hz, 1H), 7.37 (bs, 2H), 7.30-7.26 (m, 2H), 7.18-7.13 (m, 2H), 4.88-4.81 (m, 1H), 4.38 (dd, J=9.1, 5.4 Hz, 1H), 3.80-3.74 (m, 1H), 3.26-3.06 (m, 5H), 3.03-2.92 (m, 3H), 2.90-2.83 (m, 2H), 2.24-2.14 (m, 2H), 1.82-1.72 (m, 2H), 1.58-1.47 (m, 1H) ppm.

### tert-butyl (3-azidopropyl)(4-fluorophenethyl)carbamate (544)

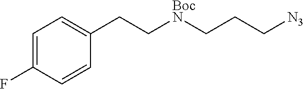 [SMILES: CC(C)(C)OC(=O)N(CCCN=[N+]=[N-])CCc1ccc(F)cc1]

2830 mg (9.51 mmol; 1.00 eq) of tert-butyl (4-fluorophenethyl)(3-hydroxypropyl)carbamate were dissolved in 40 ml of Dichloromethane. 2.64 mL of Et₃N (19.02 mmol; 2.00 eq) and 0.88 mL of Methanesulfonyl chloride (11.41 mmol; 1.20 eq) were added and the mixture was stirred over night at RT. 40 ml of a saturated NaCl-solution were added and the mixture was extracted with DCM (5×50 mL). The combined organic phases were dried over MgSO₄ and the solvent was removed under reduced pressure. The crude was used without further purification. Therefore, the residue was dissolved in 25 mL of DMF and 1854 mg (28.53 mmol; 3.00 eq) of NaN₃ were added. The mixture was heated under refluxation for 20 h. Final column chromatography (Cyclohexane/EtOAc 100:0% to 50:50%) resulted in 2600 mg (8.06 mmol; 85%) of the title compound in the form of a yellow oil. TLC: R_{f}=0.85 (Cyclohexane/EtOAc 1:1). APCI-MS (+) m/z for C₁₆H₂₃FN₄O₂: calc.: 322.38; found: 296.9 and 297.9. ¹H-NMR: (400 MHZ, DMSO-d₆) δ=7.28-7.18 (m, 2H), 7.15-7.07 (m, 2H), 3.32-3.28 (m, 4H), 3.23-3.08 (m, 2H), 2.76-2.72 (m, 2H), 1.71-1.64 (m, 2H), 1.37 and 1.33 (s, 9H) ppm.

### tert-butyl (3-aminopropyl)(4-fluorophenethyl)carbamate (545)

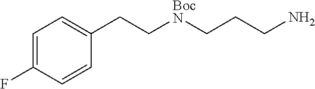 [SMILES: CC(C)(C)OC(=O)N(CCCN)CCc1ccc(F)cc1]

2600 mg (8.06 mmol; 1.00 eq) of tert-butyl (3-azidopropyl)(4-fluorophenethyl)carbamate were dissolved in 30 ml of Methanol. A catalytic amount of palladium on activated charcoal moistened with water (0.100 g; 0.05 mmol) was added and the mixture was purged with hydrogen and stirred for 10 h at rt. The catalyst was filtered off. The filtrate was concentrated under reduced pressure and the obtained crude product was purified over silica (DCM/MeOH 100:0% to 80:20%). It gave 680 mg (2.29 mmol; 28%) of the title compound in the form of a colorless oil. TLC: R_{f}=0.36 (DCM/MeOH 10:1). APCI-MS (+) m/z for C₁₆H₂₅FN₂O₂: calc.: 296.39; found: 296.9 and 297.9. ¹H-NMR: (400 MHZ, DMSO-d₆) δ=7.27-7.18 (m, 2H), 7.15-7.06 (m, 2H), 3.32-3.28 (m, 2H), 3.19-3.08 (m, 2H), 2.76-2.72 (m, 2H), 2.50-2.45 (m, 2H), 1.53-1.46 (m, 2H), 1.37 and 1.32 (s, 9H) ppm.

### tert-butyl (3-((((3aR,6R,6aS)-6-(4-amino-5-bromo-2-chloro-7H-pyrrolo[2,3-d]pyrimidin-7-yl)-2,2-dimethyltetrahydro-4H-cyclopenta[d][1,3]dioxol-4-yl)methyl)amino)propyl)(4-fluorophenethyl)carbamate (546)

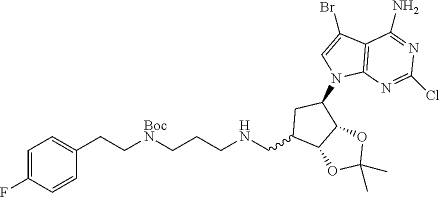 [SMILES: CC(C)(C)OC(=O)N(CCCNCC1C[C@@H](n2cc(Br)c3c(N)nc(Cl)nc32)[C@@H]2OC(C)(C)O[C@H]12)CCc1ccc(F)cc1]

160 mg (0.380 mmol; 1.00 eq) of (3aR,6R,6as)-6-(4-amino-5-bromo-2-chloro-7H-pyrrolo[2,3-d]pyrimidin-7-yl)-2,2-dimethyltetrahydro-4H-cyclopenta[d][1,3]dioxole-4-carbaldehyde were dissolved in 15 mL of DCE and reacted at RT under N₂ atmosphere with 113 mg (0.380 mmol; 1.00 eq) of tert-butyl (3-aminopropyl)(4-fluorophenethyl)carbamate as well as 121 mg (0.570 mmol; 1.50 eq) of NaB (OAc) 3H. After 19 h of stirring the solvent was removed under reduced pressure and the resulting residue was adsorbed on silica gel. Column chromatography (DCM/MeOH 100:0% to 90:10%) gave 150 mg (0.215 mmol; 57%) of the title compound in the form of a colorless foam. TLC: R_{f}=0.51 (DCM/MeOH 10:1). APCI-MS (+) m/z for C₃₁H₄₁BrClFN₆O₄: calc.: 696.06; found: 695.2, 696.2 and 697.2. ¹H-NMR: (400 MHZ, DMSO-d₆) δ=7.70 and 7.43 (s, 1H), 7.25-7.16 and 7.14-7.05 (m, 4H), 4.94-4.70 (m, 4H), 4.49-4.42 (m, 1H), 3.43-3.26 (m, 8H), 3.21-3.07 (m, 5H), 2.95-2.82 (m, 1H), 2.80-2.70 (m, 5H), 2.66-2.54 (m, 3H), 2.33-2.16 (m, 3H), 2.07-1.94 (m, 2H), 1.71-1.57 (m, 4H), 1.47 and 1.43 (s, 3H), 1.36 and 1.33 (s, 9H) 1.27 and 1.22 (s, 3H) ppm.

### (1R,2S,3R)-3-(4-amino-5-bromo-2-chloro-7H-pyrrolo[2,3-d]pyrimidin-7-yl)-5-(((3-((4-fluorophenethyl)amino)propyl)amino)methyl)cyclopentane-1,2-diol (547)

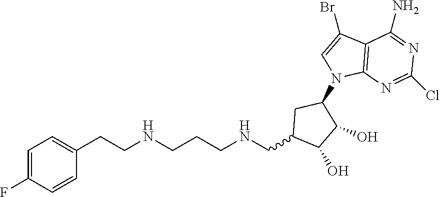 [SMILES: Nc1nc(Cl)nc2c1c(Br)cn2[C@@H]1CC(CNCCCNCCc2ccc(F)cc2)[C@@H](O)[C@H]1O]

150 mg (0.215 mmol; 1.00 eq) of tert-butyl (3-((((3aR,6R,6as)-6-(4-amino-5-bromo-2-chloro-7H-pyrrolo[2,3-d]pyrimidin-7-yl)-2,2-dimethyltetrahydro-4H-cyclopenta[d][1,3]dioxol-4-yl)methyl)amino)propyl)(4-fluorophenethyl)carbamate were dissolved in 8 mL TFA/H₂O (1:1) and stirred at RT for 24 h. The solvent was removed under reduced pressure and the residue was dissolved in methanol and purified by HPLC (MeCN/H₂O+0.1% TFA; 10/90% to 100/0% according to procedure B; t_{R}=10.06 min; UV-purity at 210, 230, 254, 242 and 275 nm >99%). 80 mg (0.102 mmol; 47%) of the title compound were obtained in the form of a yellow foam (as 2-fold TFA salt and mixture of diastereomers in a ratio of 1:1.1). APCI-MS (+) m/z for C₂₃H₂₉BrClFN₆O₂: calc.: 555.88; found: 556.8, 554.7 and 555.8. ¹H-NMR: (400 MHZ, DMSO-d₆) δ=8.81 (bs, 4H), 8.62 (bs, 2H), 8.52 (bs, 2H), 7.65 (s, 1H), 7.62 (s, 1H), 7.35-7.30 (m, 4H), 7.22-7.16 (m, 4H), 4.93-4.86 (m, 1H), 4.85-4.78 (m, 1H), 4.39-4.35 (m, 1H), 4.18-4.14 (m, 1H), 4.01-3.97 (m, 1H), 3.88-3.84 (m, 1H), 3.24-3.10 (m, 6H), 3.08-2.98 (m, 8H), 2.96-2.87 (m, 5H), 2.66-2.54 (m, 2H), 2.37-2.18 (m, 2H), 2.09-1.85 (m, 6H), 1.59-1.49 (m, 1H) ppm.

### 7-((3as,4R,6R,6aR)-6-(((tert-butyldimethylsilyl)oxy)methyl)-2,2-dimethyltetrahydro-4H-cyclopenta[d][1,3]dioxol-4-yl)-2,4-dichloro-7H-pyrrolo[2,3-d]pyrimidine (548)

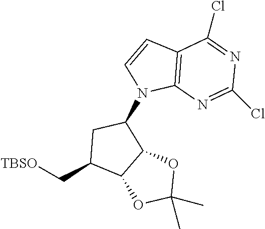 [SMILES: CC1(C)O[C@@H]2[C@@H](CO[Si](C)(C)C(C)(C)C)C[C@@H](n3ccc4c(Cl)nc(Cl)nc43)[C@@H]2O1]

1853 mg (6.56 mmol; 1.00 eq) of (3as,45,6R,6aR)-6-(((tert-butyldimethylsilyl)oxy)methyl)-2,2-dimethyltetrahydro-4H-cyclopenta[d][1,3]dioxol-4-ol were dissolved in 50 ml of Toluene and then cooled with an ice-bath to 0° C. Under continuous stirring, 3441 mg (13.12 mmol; 2.00 eq) of PPh₃ were given to the reaction mixture. 5 minutes later 3021 mg of DBAD (13.12 mmol; 2.00 eq) were added and after another 10 minutes 1233 mg (6.56 mmol; 1.00 eq) of 2-chloro-7H-pyrrolo[2,3-d]pyrimidine were given to the reaction mixture, which was then heated to 80° C. under reflux for 15 h. For purification flash chromatography was used eluting with Cyclohexane/EtOAc (99:1% to 80:20%). The desired product (1615 mg; 3.42 mmol; 52%) gave a white foam. TLC: R_{f}=0.50 (Cyclohexane/EtOAc 5:1). MS (APCI): m/z for C₂₁H₃₁Cl₂N₃O₃Si: calc.: 472.48; found 471.5. ¹H-NMR: (DMSO-d₆, 400 MHZ) δ=7.95 (d, 1H, J=4.0 Hz), 6.80 (d, 1H, J=4.0 Hz), 5.03-4.97 (m, 1H), 4.90-4.87 (m, 1H), 4.53-4.50 (m, 1H), 3.74-3.65 (m, 2H), 2.32-2.25 (m, 2H), 2.15-2.07 (m, 1H), 1.49 (s, 3H), 1.22 (s, 3H), 0.88 (s, 9H), 0.06 (s, 6H) ppm.

### 7-((3as,4R,6R,6aR)-6-(((tert-butyldimethylsilyl)oxy)methyl)-2,2-dimethyltetrahydro-4H-cyclopenta[d][1,3]dioxol-4-yl)-2-chloro-N-methyl-7H-pyrrolo[2,3-d]pyrimidin-4-amine (549)

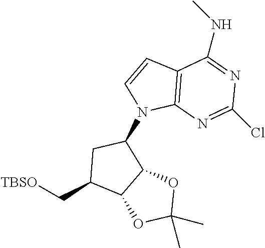 [SMILES: CNc1nc(Cl)nc2c1ccn2[C@@H]1C[C@H](CO[Si](C)(C)C(C)(C)C)[C@H]2OC(C)(C)O[C@H]21]

1615 mg (3.42 mmol; 1.00 eq) of 7-((3as,4R,6R,6aR)-6-(((tert-butyldimethylsilyl)oxy)methyl)-2,2-dimethyltetrahydro-4H-cyclopenta[d][1,3]dioxol-4-yl)-2,4-dichloro-7H-pyrrolo[2,3-d]pyrimidine were dissolved in 10 ml of Dioxane in a sealed flask. After adding 1.40 ml of a 33% solution of NH₂CH₃ in EtOH (34.20 mmol; 10.00 eq), the reaction mixture was heated to 105° C. under reflux for 17 h with stirring. Afterwards the crude was adsorbed on silica and purified by flash chromatography eluting with DCM/MeOH (100:0% to 95:5%) to obtain 910 mg (1.95 mmol; 57%) of the title compound in form of a colorless foam. TLC: R_{f}=0.90 (DCM/MeOH 10:1). ¹H-NMR: (DMSO-d₆, 400 MHZ) δ=7.28 (s, 1H), 6.59 (s, 1H), 4.88-4.79 (m, 2H), 4.50-4.47 (m, 1H), 3.72-3.63 (m, 2H), 2.93 (s, 3H), 2.26-2.16 (m, 2H), 1.99-1.96 (m, 1H), 1.47 (s, 3H), 1.21 (s, 3H), 0.88 (s, 9H), 0.05 (s, 6H) ppm.

### ((3aR,4R,6R,6as)-6-(2-chloro-4-(methylamino)-7H-pyrrolo[2,3-d]pyrimidin-7-yl)-2,2-dimethyltetrahydro-4H-cyclopenta[d][1,3]dioxol-4-yl)methanol (550)

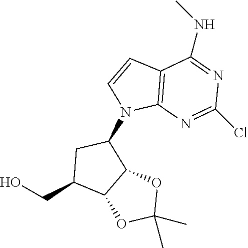 [SMILES: CNc1nc(Cl)nc2c1ccn2[C@@H]1C[C@H](CO)[C@H]2OC(C)(C)O[C@H]21]

910 mg (1.95 mmol; 1.00 eq) of 7-((3as,4R,6R,6aR)-6-(((tert-butyldimethylsilyl)oxy)methyl)-2,2-dimethyltetrahydro-4H-cyclopenta[d][1,3]dioxol-4-yl)-2-chloro-N-methyl-7H pyrrolo[2,3-d]pyrimidin-4-amine were dissolved in 10 ml of THF and thereupon 2.92 mL of a 1M solution of TBAF in THF (2.92 mmol; 1.50 eq) were added. The reaction mixture was subsequently stirred at RT for 15 h and afterwards the solvent was removed under reduced pressure. The remaining residue was purified by flash chromatography eluting with DCM/MeOH (100:0% to 90:10%) to give 650 mg (1.84 mmol; 94%) of the title compound in form of a white solid. TLC: R_{f}=0.16 (Cyclohexane/EtOAc 1:1). ¹H-NMR: (DMSO-d₆, 400 MHZ) δ=7.99-7.90 (m, 1H), 7.33 (d, 1H, J=3.6 Hz), 6.64-6.56 (m, 1H), 4.88-4.82 (m, 1H), 4.80-4.75 (m, 2H), 4.53-4.47 (m, 1H), 3.52-3.46 (m, 2H), 3.00-2.87 (m, 3H), 2.24-2.15 (m, 2H), 1.99-1.87 (m, 1H), 1.47 (s, 3H), 1.22 (s, 3H) ppm.

### 2-(((3aR,4R,6R,6as)-6-(2-chloro-4-(methylamino)-7H-pyrrolo[2,3-d]pyrimidin-7-yl)-2,2-dimethyltetrahydro-4H-cyclopenta[d][1,3]dioxol-4-yl)methyl) isoindoline-1,3-dione (551)

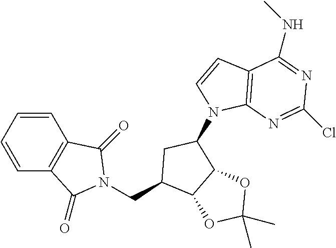 [SMILES: CNc1nc(Cl)nc2c1ccn2[C@@H]1C[C@H](CN2C(=O)c3ccccc3C2=O)[C@H]2OC(C)(C)O[C@H]21]

650 mg (1.84 mmol; 1.00 eq) of ((3aR,4R,6R,6as)-6-(2-chloro-4-(methylamino)-7H-pyrrolo[2,3-d]pyrimidin-7-yl)-2,2-dimethyltetrahydro-4H-cyclopenta[d][1,3]dioxol-4-yl)methanol were dissolved in 20 mL of THF. After cooling with an ice-bath to 0° C., 965 mg (3.68 mmol; 2.00 eq) of PPh₃ were added. The reaction mixture was flushed by N₂ gas for 5 min and reacted with 1.44 mL of a 40% solution of DEAD in Toluene (3.68 mmol; 2.00 eq). After an additional time of 5 min 541 mg (3.68 mmol; 2.00 eq) of Phthalimide were added. The reaction mixture was subsequently stirred for 16.5 h and afterwards purified by flash chromatography eluting with Cyclohexane/EtOAc (99:1% to 50:50%) to give 867 mg (1.84 mmol; quantitative) of the title compound in form of a white solid. TLC: R_{f}=0.90 (Cyclohexane/EtOAc 2:1). APCI-MS (+) m/z for C₂₄H₂₄ClN₅O₄: calc.: 481.94; found: 481.8, 482.8 and 483.7. ¹H-NMR: (DMSO-d₆, 400 MHZ) δ=7.95 (bs, 1H); 7.91-7.81 (m, 4H), 7.39 (d, 1H, J=4.0 Hz), 6.58 (bs, 1H), 4.89-4.77 (m, 2H), 4.60-4.54 (m, 1H), 3.85-3.78 (m, 1H), 3.74-3.65 (m, 1H), 2.97-2.87 (m, 3H), 2.48-2.40 (m, 1H), 2.29-2.21 (m, 1H), 2,05-1.96 (m, 1H), 1.39 (s, 3H), 1.19 (s, 3H) ppm.

### 7-((3as,4R,6R,6aR)-6-(aminomethyl)-2,2-dimethyltetrahydro-4H-cyclopenta[d][1,3]dioxol-4-yl)-2-chloro-N-methyl-7H-pyrrolo[2,3-d]pyrimidin-4-amine (552)

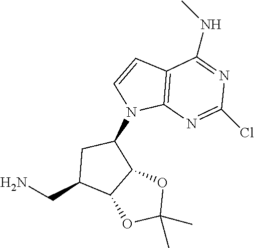 [SMILES: CNc1nc(Cl)nc2c1ccn2[C@@H]1C[C@H](CN)[C@H]2OC(C)(C)O[C@H]21]

867 mg (1.84 mmol; 1.00 eq) of 2-(((3aR,4R,6R,6as)-6-(2-chloro-4-(methylamino)-7H-pyrrolo[2,3-d]pyrimidin-7-yl)-2,2-dimethyltetrahydro-4H-cyclopenta[d][1,3]dioxol-4-yl)methyl) isoindoline-1,3-dione were dissolved in 15.0 ml of EtOH and then 0.93 mL of hydrazine hydrate (29.44 mmol; 16.0 eq) were added. The reaction mixture was heated to 90° C. under reflux and subsequently stirred for 2.5 h. Afterwards the reaction mixture was cooled to 0° C. and thereby the by-product precipitated in form of a white solid. The precipitate was filtered off, and the solvent was removed under reduced pressure. The remaining residue was purified by flash chromatography eluting with DCM/MeOH (100:0% to 85:15%) to give 211 mg (0.60 mmol; 33%) of the title compound in form of a white solid. TLC: R_{f}=0.55 (DCM/MeOH 10:1). APCI-MS (+) m/z for C₁₆H₂₂ClN₅O₂: calc.: 351.84; found: 351.8, 353.0 and 353.9. ¹H-NMR: (DMSO-d₆, 400 MHZ) δ=8.00-7.91 (m, 1H), 7.35 (d, 1H, J=4.0 Hz), 6.64-6.53 (m, 1H), 4.86-4.76 (m, 2H); 4.50-4.42 (m, 1H), 2.92 (bs, 3H), 2.75-2.70 (m, 1H), 2.66-2.61 (m, 1H), 2.27-2.19 (m, 1H), 2.14-2.06 (m, 1H), 1.93-1.79 (m, 1H), 1.47 (s, 3H), 1.22 (s, 3H) ppm

### (1s,2R,3R,5R)-3-(aminomethyl)-5-(2-chloro-4-(methylamino)-7H-pyrrolo[2,3-d]pyrimidin-7-yl)cyclopentane-1,2-diol (553)

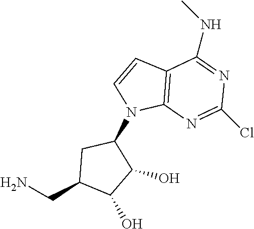 [SMILES: CNc1nc(Cl)nc2c1ccn2[C@@H]1C[C@H](CN)[C@@H](O)[C@H]1O]

347 mg (0.98 mmol; 1.00 eq) 7-((3as,4R,6R,6aR)-6-(aminomethyl)-2,2-dimethyltetrahydro-4H cyclopenta[d][1,3]dioxol-4-yl)-2-chloro-N-methyl-7H-pyrrolo[2,3-d]pyrimidin-4-amine were dissolved in a mixture of 4.0 ml of H₂O and 4.0 mL of TFA and the reaction mixture was subsequently stirred for 12 h at RT. After concentrating, the remaining residue was purified by preparative HPLC according to procedure C (MeCN/H₂O+0.1% TFA; 0/100% to 5/95%; t_{R}=8.54 min; UV-purity at 210, 230, 254, 242 and 275 nm >99%) to give the title compound (222 mg; 0.52 mmol; 53%) in form of a white foam and as 1-fold TFA salt. APCI-MS (+) m/z for C₁₃H₁₈ClN₅O₂: calc.: 311.77; found: 311.9, 313.0 and 313.9. ¹H-NMR: (DMSO-d₆, 400 MHZ) δ=8.01-7.91 (m, 1H), 7.81 (bs, 3H), 7.33-7.26 (m, 1H), 6.65-6.56 (m, 1H), 4.81-4.75 (m, 1H), 4.19-4.12 (m, 1H), 3.88-3.84 (m, 1H), 3.13-3.04 (m, 1H), 3.00-2.85 (m, 4H), 2.32-2.25 (m, 1H), 2.23-2.14 (m, 1H), 1.55-1.47 (m, 1H) ppm.

### tert-butyl (3-((((3aR,4R,6R,6as)-6-(2-chloro-4-(methylamino)-7H-pyrrolo[2,3-d]pyrimidin-7-yl)-2,2-dimethyltetrahydro-4H-cyclopenta[d][1,3]dioxol-4-yl)methyl)amino)propyl)(3-phenoxyphenethyl)carbamate (554)

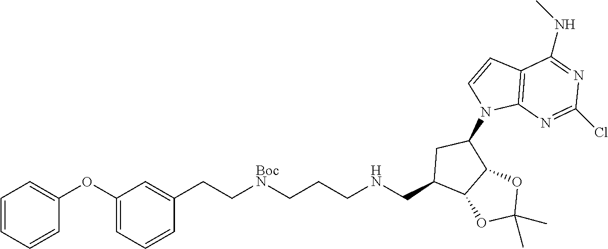 [SMILES: CNc1nc(Cl)nc2c1ccn2[C@@H]1C[C@H](CNCCCN(CCc2cccc(Oc3ccccc3)c2)C(=O)OC(C)(C)C)[C@H]2OC(C)(C)O[C@H]21]

210 mg (0.60 mmol, 1.00 eq) of 7-(3as,4R,6R,6aR)-6-(aminomethyl)-2,2-(dimethyltetrahydro-4H-cyclopenta[d][1,3]dioxol-4-yl)-2-chloro-N-methyl-7H-pyrrolo[2,3-d]pyrimidin-4-amine were dissolved in 15 mL of dry DCE. 133 mg (0.36 mmol; 0.60 eq) of (tert-butyl (3-oxopropyl)(3-phenoxyphenethyl)carbamate) and 191 mg (0.90 mmol; 1.50 eq) of NaB (OAc) 3H were added. The reaction mixture was stirred for 18 h at RT. After concentrating, the crude was purified by flash chromatography eluting with DCM/MeOH (100:0% to 90:10%). The tile compound (200 mg; 0.28 mmol; 47%) was obtained in form of a white foam. TLC: R_{f}=0.28 (DCM/MeOH 10:1). APCI-MS (+) m/z for C₃₈H₄₉ClN₆O₅: calc.: 705.30; found: 704.5, 705.7 and 706.5. ¹H-NMR: (DMSO-d₆, 400 MHZ) δ=7.99-7.89 (m, 1H), 7.40-7.34 (m, 2H), 7.33-7.31 (m, 1H), 7.24-7.29 (m, 1H), 7.14-7.09 (m, 1H), 7.02-6.92 (m, 3H), 6.87-6.79 (m, 2H), 6.61-6.55 (m, 1H), 4.87-4.76 (m, 2H), 4.47-4.41 (m, 1H), 3.53-3.42 (m, 2H), 3.36-3.27 (m, 2H), 3.18-3.05 (m, 2H), 2.97-2.89 (m, 3H), 2.78-2.62 (m, 3H), 2.60-2.53 (m, 1H), 2.30-2.13 (m, 2H), 1.92-1.82 (m, 1H), 1.62-1.55 (m, 2H), 1.45 (s, 3H), 1.35 and 1.29 (s, 9H), 1.20 (s, 3H) ppm.

### (1R,2S,3R,5R)-3-(2-chloro-4-(methylamino)-7H-pyrrolo[2,3-d]pyrimidin-7-yl)-5-(((3-((3-phenoxyphenethyl)amino)propyl)amino)methyl)cyclopentane-1,2-diol (555)

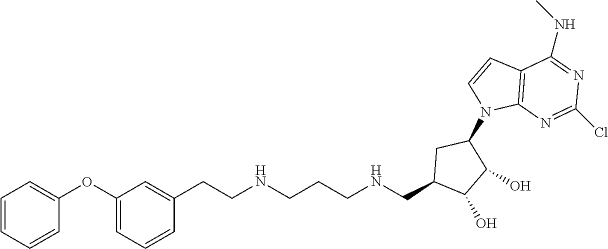 [SMILES: CNc1nc(Cl)nc2c1ccn2[C@@H]1C[C@H](CNCCCNCCc2cccc(Oc3ccccc3)c2)[C@@H](O)[C@H]1O]

200 mg (0.28 mmol; 1.00 eq) tert-butyl (3-((((3aR,4R,6R,6as)-6-(2-chloro-4-(methylamino)-7H-pyrrolo[2,3-d]pyrimidin-7-yl)-2,2-dimethyltetrahydro-4H-cyclopenta[d][1,3]dioxol-4-yl)methyl)amino)propyl)(3-phenoxyphenethyl)carbamate were dissolved in a mixture of 4.0 ml of H₂O and 4.0 mL of TFA and the reaction mixture was subsequently stirred for 16.5 h at RT. After concentrating, the remaining residue was purified by preparative HPLC according to procedure A (MeCN/H₂O+0.1% TFA; 10/90% to 100/0%; t_{R}=14.66 min; UV-purity at 210, 230, 254, 242 and 275 nm >99%) to give the title compound (102 mg; 0.129 mmol; 46%) in form of a white foam and as 2-fold TFA salt. APCI-MS (+) m/z for C₃₀H₃₇ClN₆O₃: calc.: 565.12; found: 564.8, 565.8 and 566.7. ¹H-NMR: (DMSO-d₆, 400 MHZ) δ=8.79 (bs, 2H), 8.61 (bs, 2H), 8.00-7.94 (m, 1H), 7.45-7.40 (m, 2H), 7.37 (d, 1H, J=7.9 Hz), 7.30-7.27 (m, 1H), 7.19-7.15 (m, 1H), 7.09-7.06 (m, 1H), 7.05-7.01 (m, 2H), 6.99-6.97 (m, 1H), 6.93-6-89 (m, 1H), 6.64-6.57 (m, 1H), 4.81-4.74 (m, 1H), 4.20-4-14 (m, 1H), 3.91-3-86 (m, 1H), 3.25-3.14 (m, 3H), 3.11-2.99 (m, 5H), 2.97-2.88 (m, 5H), 2.37-2.30 (m, 1H), 2.28-2.21 (m, 1H), 2.01-1.94 (m, 2H), 1.60-1.52 (m, 1H) ppm.

### 2,4-dichloro-5-iodo-7H-pyrrolo[2,3-d]pyrimidine (556)

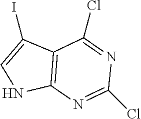 [SMILES: Clc1nc(Cl)c2c(I)c[nH]c2n1]

1880 mg (10.00 mmol; 1.00 eq) 2,4-dichloro-7H-pyrrolo[2,3-d]pyrimidine were dissolved in 40 ml of DMF. 2475 mg (11.00 mmol; 1.10 eq) of NIS were added and after 16 h of stirring at RT the reaction mixture was quenched with 100 ml of a solution of saturated NaHCO₃ and then extracted with EtOAc (6×50 mL). The organic phases were combined, dried with MgSO₄, and purified by flash chromatography eluting with CH/EtOAc (99:1% to 50:50%) to obtain compound (3021 mg; 8.43 mmol; 84%) in form a yellow solid. TLC: R_{f}=0.53 (Cyclohexane/EtOAc 1:1). APCI-MS (+) m/z for C₆H₂Cl₂IN₃: calc.: 313.91; found: 313.6, 315.6 and 316.7. ¹H-NMR: (DMSO-d₆, 400 MHZ) δ=13.12 (s, 1H); 7.98 (s, 1H) ppm.

### 7-((3as,4R,6R,6aR)-6-(((tert-butyldimethylsilyl)oxy)methyl)-2,2-dimethyltetrahydro-4H-cyclopenta[d][1,3]dioxol-4-yl)-2,4-dichloro-5-iodo-7H-pyrrolo[2,3-d]pyrimidine (557)

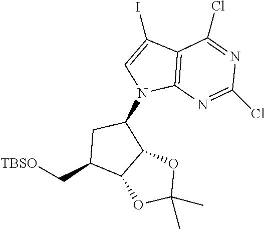 [SMILES: CC1(C)O[C@@H]2[C@@H](CO[Si](C)(C)C(C)(C)C)C[C@@H](n3cc(I)c4c(Cl)nc(Cl)nc43)[C@@H]2O1]

2056 mg (6.80 mmol; 1.00 eq) of (3as,45,6R,6aR)-6-(((tert-butyldimethylsilyl)oxy)methyl)-2,2-dimethyltetrahydro-4H-cyclopenta[d][1,3]dioxol-4-ol were dissolved in 50 ml of Toluene and then cooled with an ice-bath to 0° C. 3567 mg (13.60 mmol; 2.00 eq) of PPh₃ were given to the reaction mixture. After 5 minutes 3132 mg (13.60 mmol; 2.00 eq) of DBAD were added. After 10 more minutes compound 2437 mg (6.80 mmol; 1.00 eq) of 2,4-dichloro-5-iodo-7H-pyrrolo[2,3-d]pyrimidine were added. The reaction mixture was then heated to 80° C. under reflux for 14 h and thereby stirred continuously. The crude was purified by flash chromatography eluting with Cyclohexane/EtOAc (99:1% to 80:20%) to obtain the title compound (1818 mg; 3.04 mmol; 45%) in form of a purple foam. TLC: R$ =0.47 (Cyclohexane/EtOAc 5:1). APCI-MS (+) m/z for for C₂₁H₃₀Cl₂ IN₃O₃Si: calc.: 598.38; found 598.0, 599.0 and 600.0. ¹H-NMR: (DMSO-d₆, 400 MHZ) δ=8.20 (s, 1H), 5.02-4.96 (m, 1H), 4.89-4.86 (m, 1H), 4.51-4.46 (m, 1H), 3.73-3.62 (m, 2H), 2.32-2.21 (m, 2H), 2.11-2.02 (m, 1H), 1.46 (s, 3H), 1.21 (s, 3H), 0.88 (s, 9H), 0.06 (s, 6H) ppm.

### 7-((3as,4R,6R,6aR)-6-(((tert-butyldimethylsilyl)oxy)methyl)-2,2-dimethyltetrahydro-4H-cyclopenta[d][1,3]dioxol-4-yl)-?-chloro-5-indo-7H-pyrrolo[2,3-d]pyrimidin-4-amine (558)

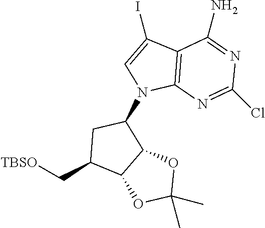 [SMILES: CC1(C)O[C@@H]2[C@@H](CO[Si](C)(C)C(C)(C)C)C[C@@H](n3cc(I)c4c(N)nc(Cl)nc43)[C@@H]2O1]

1818 mg (3.04 mmol; 1.00 eq) of 7-((3as,4R,6R,6aR)-6-(((tert-butyldimethylsilyl)oxy)methyl)-2,2-dimethyltetrahydro-4H-cyclopenta[d][1,3]dioxol-4-yl)-2,4-dichloro-5-iodo-7H-pyrrolo[2,3-d]pyrimidin were dissolved in 10 ml of Dioxane. Then 40 mL of a 25% solution of NH₃ in H₂O were added. Afterwards the reaction mixture was heated in a sealed flask to 105° C. for 62 h with stirring. The solvent was removed under reduced pressure and the remaining residue was purified by flash chromatography eluting with DCM/MeOH (100:0% to 95:5%) to give the title compound (1240 mg; 2.14 mmol; 71%) in form of purple foam. TLC: R_{f}=0.46 (DCM/MeOH 20:1). MS (APCI): calctd. for C₂₁H₃₂ClIN₄O₃Si: calc.: 578.10; found 578.6. ¹H-NMR: (DMSO-d₆, 400 MHZ) δ=7.63 (s, 1H), 4.87-4.79 (m, 2H), 4.47-4.44 (m, 1H), 3.71-3.61 (m, 2H), 2.24-2.17 (m, 2H), 1.99-1.94 (m, 1H), 1.46 (s, 3H), 1.21 (s, 3H), 0.87 (s, 9H), 0.05 (s, 6H) ppm.

### 7-((3as,4R,6R,6aR)-6-(((tert-butyldimethylsilyl)oxy)methyl)-2,2-dimethyltetrahydro-4H-cyclopenta[d][1,3]dioxol-4-yl)-2-chloro-5-phenyl-7H-pyrrolo[2,3-d]pyrimidin-4-amine (559)

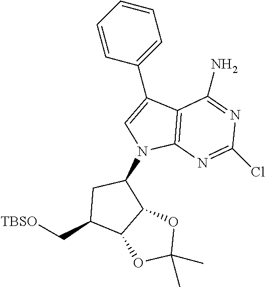 [SMILES: CC1(C)O[C@@H]2[C@@H](CO[Si](C)(C)C(C)(C)C)C[C@@H](n3cc(-c4ccccc4)c4c(N)nc(Cl)nc43)[C@@H]2O1]

654 mg (1.13 mmol; 1.00 eq) of 7-((3as,4R,6R,6aR)-6-(((tert-butyldimethylsilyl)oxy)methyl)-2,2-dimethyltetrahydro-4H-cyclopenta[d][1,3]dioxol-4-yl)-2-chloro-5-iodo-7H-pyrrolo[2,3-d]pyrimidin-4-amine, 480 mg of K₃PO₄ (2.26 mmol; 2.00 eq) and 237 mg of phenylboronic acid (1.94 mmol; 1.72 eq) were dissolved in a mixture of 10.0 ml of Dioxane and 10.0 ml of degassed H₂O. Then the catalyst Pd (dtbpf) Cl₂ was added (65 mg; 0.11 mmol; 0.10 eq) under N₂-atmosphere. The reaction mixture was heated to 90° C. under reflux with stirring. After 45 min 100 ml of a solution of saturated NaHCO₃were added and the reaction mixture was extracted with EtOAc (5×50 mL). The organic phases were combined, dried with MgSO₄, and then purified by flash chromatography eluting with Cyclohexane/EtOAc (99:1% to 80:20%) to give the title compound (472 mg; 0.89 mmol; 79%) in form of colorless foam. TLC: R_{f}=0.48 (Cyclohexane/EtOAc 3:1). MS (APCI): m/z for C₂₇H₃₇ClNAO3Si: calc.: 529.15; found 528.5. ¹H-NMR: (DMSO-d₆, 400 MHZ) δ=7.51 (s, 1H), 7.48-7.34 (m, 5H) 4.98-4.88 (m, 2H), 4.50-4.48 (m, 1H), 3.73-3.63 (m, 2H), 2.30-2.23 (m, 2H), 2.08-2.01 (m, 1H), 1.48 (s, 3H), 1.22 (s, 3H), 0.87 (s, 9H), 0.05 (s, 6H) ppm.

### ((3aR,4R,6R,6as)-6-(4-amino-2-chloro-5-phenyl-7//pyrrolo[2,3-d]pyrimidin-7-yl)-2,2-dimethyltetrahydro-4H-cyclopenta[d][1,3]dioxol-4-yl)methanol (560)

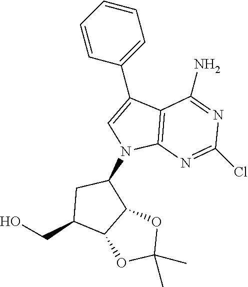 [SMILES: CC1(C)O[C@@H]2[C@@H](CO)C[C@@H](n3cc(-c4ccccc4)c4c(N)nc(Cl)nc43)[C@@H]2O1]

472 mg (0.89 mmol; 1.00 eq) of 7-((3as,4R,6R,6aR)-6-(((tert-butyldimethylsilyl)oxy)methyl)-2,2-dimethyltetrahydro-4H-cyclopenta[d][1,3]dioxol-4-yl)-2-chloro-5-phenyl-7H-pyrrolo[2,3-d]pyrimidin-4-amine were dissolved in 10.0 ml of THF and 1.34 mL of a 1 M solution of TBAF in THF (1.34 mmol; 1.50 eq) were added. After 14.5 h of stirring the reaction mixture was purified by flash chromatography eluting with DCM/MeOH (100:0% to 95:5%) to give the title compound (333 mg; 0.80 mmol; 90%) in form of colorless foam. TLC: R_{f}=0.57 (DCM/MeOH 10:1). MS (APCI): m/z for C₂₁H₂₃ClN₄O₃: calc.: 414.89; found: 414.8. ¹H-NMR: (DMSO-d₆, 400 MHZ) δ=7.57 (s, 1H), 7.51-7.45 (m, 4H), 7.40-7.33 (m, 1H), 5.00-4.91 (m, 1H), 4.89-4.84 (m, 1H), 4.77 (t, 1H, J=6.0 Hz), 4.54-4.47 (m, 1H), 3.54-3.46 (m, 2H), 2.32-2.18 (m, 2H), 2.04-1.94 (m, 1H), 1.48 (s, 3H); 1.23 (s, 3H) ppm.

### 2-(((3aR,4R,6R,6as)-6-(4-amino-2-chloro-5-phenyl-7H-pyrrolo[2,3-d]pyrimidin-7-yl)-2,2-dimethyltetrahydro-4H-cyclopenta[d][1,3]dioxol-4-yl)methyl) isoindoline-1,3-dione (561)

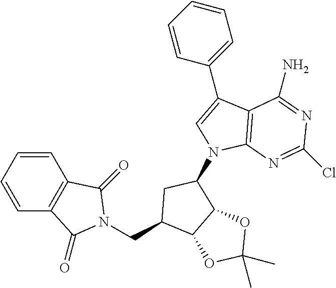 [SMILES: CC1(C)O[C@@H]2[C@@H](CN3C(=O)c4ccccc4C3=O)C[C@@H](n3cc(-c4ccccc4)c4c(N)nc(Cl)nc43)[C@@H]2O1]

333 mg (0.80 mmol; 1.00 eq) of ((3aR,4R,6R,6as)-6-(4-amino-2-chloro-5-phenyl-7H-pyrrolo[2,3-d]pyrimidin-7-yl)-2,2-dimethyltetrahydro-4H-cyclopenta[d][1,3]dioxol-4-yl)methanol were dissolved in 20 ml of THF. After cooling with an ice-bath to 0° C., 420 mg of PPh₃ (1.60 mmol; 2.00 eq) were added. Then the reaction mixture was flushed with N₂ gas for 5 min and after 5 more minutes, 0.63 mL of a 40% solution of DEAD in toluene (1.60 mmol; 2.00 eq) were given to the reaction mixture. After an additional time of 5 min, 235 mg of phthalimide (1.60 mmol; 2.00 eq) were added, whereby the entry of O₂ into the flask was prevented as far as possible. The reaction mixture was subsequently stirred for 16.5 h and afterwards purified by flash chromatography eluting with Cyclohexane/EtOAc (99:1% to 70:30%) to give the title compound (321 mg; 0.59 mmol; 74%) in form of white solid. TLC:

R_{f}=0.37 (Cyclohexane/EtOAc 7:3). MS (APCI): m/z for C₂₉H₂₆ClN₅₀₄: calc.: 544.01; found 543.7.

¹H-NMR: (DMSO-d₆, 400 MHZ) δ=7.92-7.87 (m, 1H), 7.86-7.81 (m, 1H), 7.66 (s, 1H), 7.51-7.46 (m, 4H), 7.41-7.34 (m, 1H), 5.00-4.92 (m, 1H), 4.90-4.86 (m, 1H), 4.62-4.55 (m, 1H), 3.87-3.79 (m, 1H), 3.75-3.68 (m, 1H), 2.37-2.28 (m, 1H), 2.14-2.04 (m, 1H), 1.42 (s, 3H), 1.17 (m, 3H) ppm; signal of one Proton overlayed by the DMSO-peak.

### 7-((3as,4R,6R,6aR)-6-(aminomethyl)-2,2-dimethyltetrahydro-4H-cyclopenta[d][1,3]dioxol-4-yl)-2-chloro-5-phenyl-7H-pyrrolo[2,3-d]pyrimidin-4-amine (562)

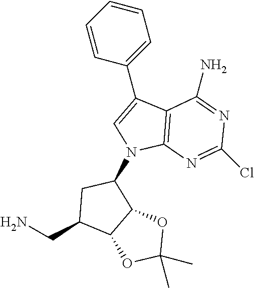 [SMILES: CC1(C)O[C@@H]2[C@@H](CN)C[C@@H](n3cc(-c4ccccc4)c4c(N)nc(Cl)nc43)[C@@H]2O1]

321 mg (0.59 mmol; 1.00 eq) of 2-(((3aR,4R,6R,6as)-6-(4-amino-2-chloro-5-phenyl-7H-pyrrolo[2,3-d]pyrimidin-7-yl)-2,2-dimethyltetrahydro-4H-cyclopenta[d][1,3]dioxol-4-yl)methyl) isoindoline-1,3-dione were dissolved in 15.0 mL of EtOH and then 0.30 ml of hydrazine hydrate (9.44 mmol; 16.00 eq) were added. The reaction mixture was heated to 90° C. under reflux and subsequently stirred for 35 min. Afterwards, the reaction mixture was cooled to 0° C., and thereby the by-product precipitated in form of a white solid. The precipitate was filtered off, and the solvent was removed under reduced pressure. The remaining residue was purified by flash chromatography eluting with DCM/MeOH (100:0% to 90:10%) to give the title compound (200 mg; 0.48 mmol; 82%) in form of a white solid. TLC: R_{f}=0.55 (DCM/MeOH 10:1). MS (APCI): m/z for C₂₁H₂₄ClN₅O₂: calc.: 413.91; found 413.8. ¹H-NMR: (DMSO-d₆, 400 MHZ) δ=7.50-7.46 (m, 5H), 7.39-7.34 (m, 1H), 4.99-4.84 (m, 2H), 4.52-4.47 (m, 1H), 3.49-3.22 (m, 2H), 2.78-2.64 (m, 1H), 2.38-2.26 (m, 2H), 1.49 (m, 3H), 1.23 (s, 3H) ppm.

### tert-butyl (3-((((3aR,4R,6R,6as)-6-(4-amino-2-chloro-5-phenyl-7H-pyrrolo[2,3-d]pyrimidin-7-yl)-2,2-dimethyltetrahydro-4H-cyclopenta[d][1,3]dioxol-4-yl)methyl)amino)propyl)(3-phenoxyphenethyl)carbamate (563)

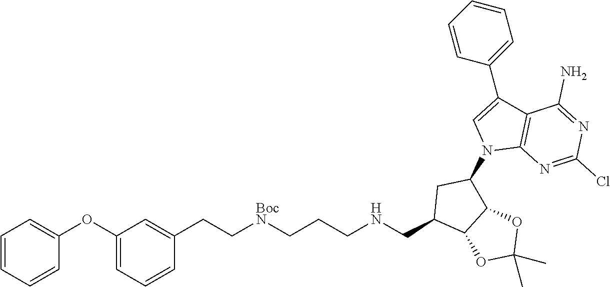 [SMILES: CC(C)(C)OC(=O)N(CCCNC[C@H]1C[C@@H](n2cc(-c3ccccc3)c3c(N)nc(Cl)nc32)[C@@H]2OC(C)(C)O[C@H]12)CCc1cccc(Oc2ccccc2)c1]

200 mg of 7-((3as,4R,6R,6aR)-6-(aminomethyl)-2,2-dimethyltetrahydro-4H cyclopenta[d][1,3]dioxol-4-yl)-2-chloro-5-phenyl-7H-pyrrolo[2,3-d]pyrimidin-4-amine (0.48 mmol; 1.00 eq) were dissolved in 15 mL of dry DCE. 115 mg of tert-butyl (3-oxopropyl)(3-phenoxyphenethyl)carbamate (0.31 mmol; 0.65 eq) and 153 mg of NaB (OAc) 3H (0.72 mmol; 1.50 eq) were added. The reaction mixture was stirred for 18 h at RT. Afterwards the crude was purified by flash chromatography eluting with DCM/MeOH (100:0% to 90:10%). The title compound was yielded in form of a colorless foam (120 mg; 0.16 mmol; 50%). TLC: R_{f}=0.39 (DCM/MeOH 10:1). APCI-MS (+) m/z for C₄₃H₅₁ClN₆O₅: calc.: 767.37; found: 766.5, 767.6 and 768.5. ¹H-NMR: (DMSO-d₆, 400 MHZ) δ=7.57 (s, 1H), 7.50-7.43 (m, 4H), 7.41-7.34 (m, 3H), 7.29-7.22 (m, 1H), 7.14-7.08 (m, 1H), 7.00-6.92 (m, 3H), 6.86-6.78 (m, 2H), 4.96-4.90 (m, 1H), 4.88-4.83 (m, 1H), 4.48-4.42 (m, 1H), 3.38-3.25 (m, 4H), 3.19-3.04 (m, 2H), 2.77-2.69 (m, 2H), 2.68-2.63 (m, 1H), 2.59-2.53 (m, 1H), 2.35-2.26 (m, 1H), 2.24-2.15 (m, 1H), 2.00-1.91 (m, 1H); 1.62-1.51 (m, 2H), 1.47 (s, 3H), 1.34 and 1.29 (s, 9H), 1.21 (s, 3H) ppm.

### (1R,2S,3R,5R)-3-(4-amino-2-chloro-5-phenyl-7//pyrrolo[2,3-d]pyrimidin-7-yl)-5-(((3-((3-phenoxyphenethyl)amino)propyl)amino)methyl)cyclopentane-1,2-diol (564)

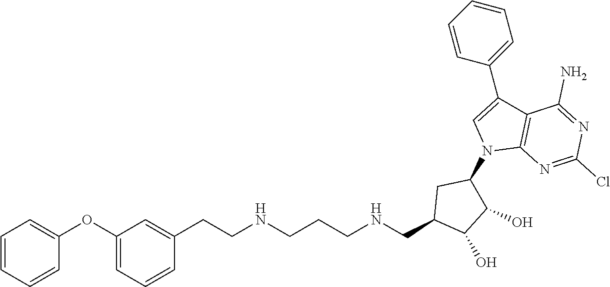 [SMILES: Nc1nc(Cl)nc2c1c(-c1ccccc1)cn2[C@@H]1C[C@H](CNCCCNCCc2cccc(Oc3ccccc3)c2)[C@@H](O)[C@H]1O]

120 mg of tert-butyl (3-((((3aR,4R,6R,6as)-6-(4-amino-2-chloro-5-phenyl-7H-pyrrolo[2,3-d]pyrimidin-7-yl)-2,2-dimethyltetrahydro-4H-cyclopenta[d][1,3]dioxol-4-yl)methyl)amino)propyl)(3-phenoxyphenethyl)carbamate (0.156 mmol; 1.00 eq) were dissolved in a mixture of 4.0 mL of H₂O and 4.0 mL of TFA and the reaction mixture was subsequently stirred for 17 h. After removing the solvent under reduced pressure, the remaining residue was purified by preparative HPLC according to procedure A (MeCN/H₂O+0.1% TFA; 10/90% to 100/0%; t_{R}=16.13 min; UV-purity at 210, 230, 254, 242 and 275 nm >99%) to give the title compound (97 mg; 0.113 mmol; 73%), which appeared in form of a white foam and as 2-fold TFA salt. APCI-MS (+) m/z for C₃₅H₃₉ClN₆O₃: calc.: 627.19; found: 626.9, 627.9 and 628.8. ¹H-NMR: (DMSO-d₆, 400 MHZ) δ=8.81 (s, 2H), 8.64 (s, 2H), 7.54-7.46 (m, 5H), 7.46-7.36 (m, 4H), 7.20-7.15 (m, 1H), 7.09-7.06 (m, 1H), 7.05-7.03 (m, 1H), 7.02-7.00 (m, 1H), 6.99-6.96 (m, 1H), 6.94-6.88 (m, 1H), 4.94-4.85 (m, 1H), 4.28-4.21 (m, 1H), 3.90 (t, 1H, J=5.3 Hz), 3.26-3.14 (m, 3H), 3.12-2.99 (m, 5H), 2.96-2.88 (m, 2H), 2.42-2.34 (m, 1H), 2.32-2.22 (m, 1H) 2.04-1.94 (m, 2H), 1.66-1.56 (m, 1H) ppm.

### tert-butyl (3-((((3aR,4R,6R,6as)-6-(4-amino-2-chloro-5-phenyl-7H-pyrrolo[2,3-d]pyrimidin-7-yl)-2,2-dimethyltetrahydro-4H-cyclopenta[d][1,3]dioxol-4-yl)methyl)amino)propyl)(4-fluorophenethyl)carbamate (565)

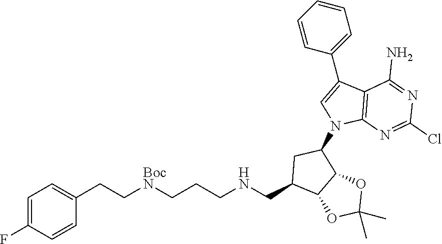 [SMILES: CC(C)(C)OC(=O)N(CCCNC[C@H]1C[C@@H](n2cc(-c3ccccc3)c3c(N)nc(Cl)nc32)[C@@H]2OC(C)(C)O[C@H]12)CCc1ccc(F)cc1]

100 mg (0.240 mmol; 1.00 eq) of 7-((3as,4R,6R,6aR)-6-(aminomethyl)-2,2-dimethyltetrahydro-4H cyclopenta[d][1,3]dioxol-4-yl)-2-chloro-5-phenyl-7H-pyrrolo[2,3-d]pyrimidin-4-amine were dissolved in 15 ml of DCE and reacted at RT under N₂ atmosphere with 62 mg (0.210 mmol; 0.90 eq) of tert-butyl (4-fluorophenethyl)(3-oxopropyl)carbamate as well as 76 mg (0.360 mmol; 1.50 eq) of NaB (OAc) 3H. After 14 h of stirring the solvent was removed under reduced pressure and the resulting residue was adsorbed on silica gel. Column chromatography (DCM/MeOH 100:0% to 90:10%) gave 120 mg (0.173 mmol; 71%) of the title compound in the form of a colorless foam. TLC: R$ =0.47 (DCM/MeOH 10:1). APCI-MS (+) m/z for C₃₇H₄₆ClFN₆O₄: calc.: 693.26; found: 692.6 and 694.5. ¹H-NMR: (DMSO-d₆, 400 MHZ) δ=7.58 (s, 1H), 7.51-7.45 (m, 4H), 7.40-7.34 (m, 1H), 7.23-7.16 (m, 2H), 7.13-7.04 (m, 2H), 4.99-4.90 (m, 1H), 4.89-4.85 (m, 1H), 4.50-4.43 (m, 1H), 3.20-3.09 (m, 2H), 2.77-2.70 (m, 2H), 2.65-2.55 (m, 2H), 2.31-2.18 (m, 2H), 2.01-1.94 (m, 1H), 1.66-1.54 (m, 2H), 1.48 (s, 3H), 1.36 and 1.31 (s, 9H), 1.22 (s, 3H) ppm; signal of four Protons overlayed by the H₂O-peak.

### (1R,2S,3R,5R)-3-(4-amino-2-chloro-5-phenyl-7/pyrrolo[2,3-d]pyrimidin-7-yl)-5-(( (3-((4-fluorophenethyl)amino)propyl)amino)methyl)cyclopentane-1,2-diol (566)

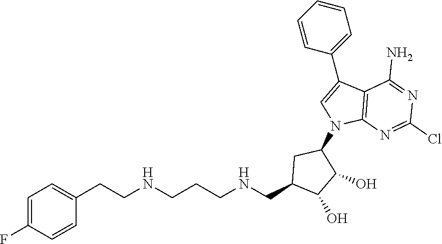 [SMILES: Nc1nc(Cl)nc2c1c(-c1ccccc1)cn2[C@@H]1C[C@H](CNCCCNCCc2ccc(F)cc2)[C@@H](O)[C@H]1O]

80 mg (0.115 mmol; 1.00 eq) of tert-butyl (3-((((3aR,4R,6R,6as)-6-(4-amino-2-chloro-5-phenyl-7H pyrrolo[2,3-d]pyrimidin-7-yl)-2,2-dimethyltetrahydro-4H-cyclopenta[d][1,3]dioxol-4-yl)methyl)amino)propyl)(4-fluorophenethyl)carbamate were dissolved in a mixture of 5.0 ml of H₂O and TFA (4:1) and the reaction mixture was subsequently stirred at RT for 16 h. After removing the solvent under reduced pressure, the resulting product was purified via preparative HPLC according to procedure A (MeCN/H₂O+0.1% TFA; 10/90% to 100/0%; t_{R}=13.46 min; UV-purity at 210, 230, 254, 242 and 275 nm >96%) to give the title compound (36 mg; 0.046 mmol; 40%) in form of a white foam and as 2-fold TFA salt. APCI-MS (+) m/z for C₂₉H₃₄ClFN₆O₂: calc.: 553.08; found: 552.6, 553.9 and 554.6. ¹H-NMR: (DMSO-d₆, 400 MHZ) δ=8.80 (s, 2H), 8.61 (s, 2H), 7.54-7.44 (m, 5H), 7.40-7.35 (m, 1H), 7.34-7.28 (m, 2H), 7.21-7.15 (m, 2H), 4.91-4.84 (m, 1H), 4.25-4.21 (m, 1H), 3.91-3.87 (m, 1H), 3.25-3.11 (m, 3H), 3.10-2.97 (m, 5H), 2.97-2.86 (m, 2H), 2.40-2.31 (m, 1H), 2.30-2.21 (m, 1H), 2.01-1.92 (m, 2H), 1.64-1.56 (m, 1H) ppm.

### tert-butyl (3-((((3aR,6R,6as)-6-(4-amino-2-chloro-5-phenyl-7H-pyrrolo[2,3-d]pyrimidin-7-yl)-2,2-dimethyltetrahydro-4H-cyclopenta[d][1,3]dioxol-4-yl)methyl)(ethyl)amino)propyl)(4-fluorophenethyl)carbamate (567)

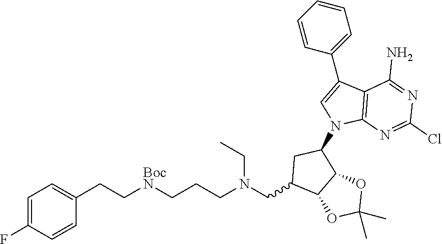 [SMILES: CCN(CCCN(CCc1ccc(F)cc1)C(=O)OC(C)(C)C)CC1C[C@@H](n2cc(-c3ccccc3)c3c(N)nc(Cl)nc32)[C@@H]2OC(C)(C)O[C@H]12]

100 mg (0.144 mmol; 1.00 eq) of tert-butyl (3-((((3aR,4R,6R,6as)-6-(4-amino-2-chloro-5-phenyl-7H-pyrrolo[2,3-d]pyrimidin-7-yl)-2,2-dimethyltetrahydro-4H-cyclopenta[d][1,3]dioxol-4-yl)methyl)amino)propyl)(4-fluorophenethyl)carbamate were dissolved in 15 mL of DCE and reacted at RT under N₂ atmosphere with 12 μL (0.217 mmol; 1.20 eq) of Acetaldehyde as well as 46 mg (0.129 mmol; 1.50 eq) of NaB (OAc) 3H. After 20 h of stirring the solvent was removed under reduced pressure and the resulting residue was adsorbed on silica gel. Column chromatography (DCM/MeOH 100:0% to 90:10%) gave 70 mg (0.097 mmol; 67%) of the title compound in the form of a colorless foam and mixture of diastereomers (in a ratio of 1:0.66). TLC: R_{f}=0.51 (DCM/MeOH 10:1). APCI-MS (+) m/z for C₃₉H₅₀ClFN₆O₄: calc.: 721.32; found: 720.5, 721.7 and 722.4.

### (1R,2S,3R,5R)-3-(4-amino-2-chloro-5-phenyl-7//-pyrrolo[2,3-d]pyrimidin-7-yl)-5-((ethyl(3-((4-fluorophenethyl)amino)propyl)amino)methyl)cyclopentane-1,2-diol (568)

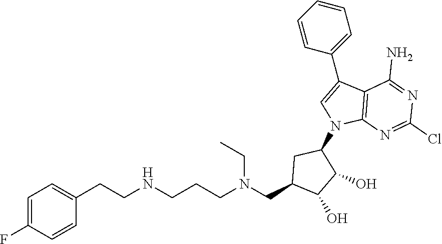 [SMILES: CCN(CCCNCCc1ccc(F)cc1)C[C@H]1C[C@@H](n2cc(-c3ccccc3)c3c(N)nc(Cl)nc32)[C@H](O)[C@@H]1O]

70 mg (0.097 mmol; 1.00 eq) of tert-butyl (3-((((3aR,6R,6as)-6-(4-amino-2-chloro-5-phenyl-7H-pyrrolo[2,3-d]pyrimidin-7-yl)-2,2-dimethyltetrahydro-4H-cyclopenta[d][1,3]dioxol-4-yl)methyl)(ethyl)amino)propyl)(4-fluorophenethyl)carbamate were dissolved in a mixture of 5.0 ml of H₂O and TFA (4:1) and the reaction mixture was subsequently stirred at RT for 18 h. After removing the solvent under reduced pressure, the resulting product was purified via preparative HPLC according to procedure A (MeCN/H₂O+0.1% TFA; 10/90% to 100/0%;

t_{R}=13.31 min; UV-purity at 210, 230, 254, 242 and 275 nm >99%) to give the title compound as main product (34 mg; 0.042 mmol; 43%) in form of a white foam and as 2-fold TFA salt. APCI-MS (+) m/z for C₃₁H₃₈ClFN₆O₂: calc.: 581.13; found: 580.8, 581.8 and 582.8. ¹H-NMR: (DMSO-d₆, 400 MHZ) δ=9.42 (s, 1H), 8.78 (s, 2H), 7.52-7.43 (m, 5H), 7.40-7.35 (m, 1H), 7.34-7.28 (m, 2H), 7.22-7.14 (m, 2H), 4.94-4.83 (m, 1H), 4.20 (t, J=6.3 Hz, 1H), 3.90-3.85 (m, 1H), 3.40-3.30 (m, 1H), 3.22-3.12 (m, 7H), 3.03 (bs, 2H), 2.94-2.87 (m, 2H), 2.43-2.27 (m, 2H), 2.07-1.93 (m, 2H), 1.67-1.57 (m, 1H), 1.24 (t, J=7.2 Hz, 3H) ppm.

### tert-butyl (3-((((3aR,6R,6as)-6-(4-amino-2-chloro-5-phenyl-7H-pyrrolo[2,3-d]pyrimidin-7-yl)-2,2-dimethyltetrahydro-4H-cyclopenta[d][1,3]dioxol-4-yl)methyl)(isopropyl)amino)propyl)(4-fluorophenethyl)carbamate (569)

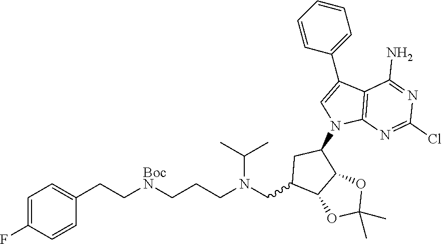 [SMILES: CC(C)N(CCCN(CCc1ccc(F)cc1)C(=O)OC(C)(C)C)CC1C[C@@H](n2cc(-c3ccccc3)c3c(N)nc(Cl)nc32)[C@@H]2OC(C)(C)O[C@H]12]

100 mg (0.144 mmol; 1.00 eq) of tert-butyl (3-((((3aR,6R,6as)-6-(4-amino-2-chloro-5-phenyl-7H-pyrrolo[2,3-d]pyrimidin-7-yl)-2,2-dimethyltetrahydro-4H-cyclopenta[d][1,3]dioxol-4-yl)methyl)(isopropyl)amino)propyl)(4-fluorophenethyl)carbamate were dissolved in 15 mL of DCE and reacted at RT under N₂ atmosphere with 16 μL (0.217 mmol; 1.20 eq) of Acetone as well as 46 mg (0.129 mmol; 1.50 eq) of NaB (OAc) 3H. After 22 h of stirring the solvent was removed under reduced pressure and the resulting residue was adsorbed on silica gel. Column chromatography (DCM/MeOH 100:0% to 95:5%) gave 80 mg (0.108 mmol; 75%) of the title compound in the form of a colorless foam and mixture of diastereomers (in a ratio of 1:0.35). TLC: R_{f}=0.18 (DCM/MeOH 20:1). APCI-MS (+) m/z for C₄₀H₅₂ClFN₆₀₄: calc.: 735.34; found: 734.7, 735.7 and 736.7.

### (1R,2S,3R,5R)-3-(4-amino-2-chloro-5-phenyl-7H-pyrrolo[2,3-d]pyrimidin-7-yl)-5-(((3-((4-fluorophenethyl)amino)propyl)(isopropyl)amino)methyl)cyclopentane-1,2-diol (570)

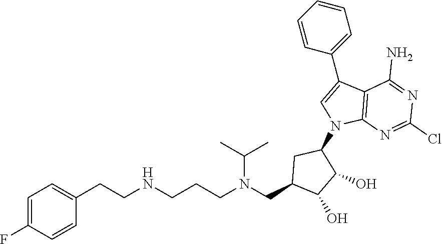 [SMILES: CC(C)N(CCCNCCc1ccc(F)cc1)C[C@H]1C[C@@H](n2cc(-c3ccccc3)c3c(N)nc(Cl)nc32)[C@H](O)[C@@H]1O]

80 mg (0.108 mmol; 1.00 eq) of tert-butyl (3-((((3aR,4R,6R,6as)-6-(4-amino-2-chloro-5-phenyl-7H-pyrrolo[2,3-d]pyrimidin-7-yl)-2,2-dimethyltetrahydro-4H-cyclopenta[d][1,3]dioxol-4-yl)methyl)(isopropyl)amino)propyl)(4-fluorophenethyl)carbamate were dissolved in a mixture of 5.0 mL of H₂O and TFA (4:1). The reaction mixture was stirred for 24 h at RT. Afterwards the solvent was removed under reduced pressure and the remaining residue was purified using HPLC according to procedure A (MeCN/H₂O+0.1% TFA; 10/90% to 100/0%; t_{R}=14.48 min; UV-purity at 210, 230, 254, 242 and 275 nm >97%) to give the title compound as main product (40 mg; 0.049 mmol; 45%) in form of a white foam and as 2-fold TFA salt. APCI-MS (+) m/z for C₃₂H₄₀ClFN₆O₂: calc.: 595.16; found: 594.5, 595.8 and 596.7. ¹H-NMR: (DMSO-d₆, 400 MHZ) δ=9.04 (s, 1H), 8.86 (s, 2H), 7.54-7.44 (m, 5H), 7.42-7.36 (m, 1H), 7.35-7.29 (m, 2H), 7.23-7.15 (m, 2H), 4.93-4.86 (m, 1H), 4.25-4.21 (m, 1H), 3.92-3.86 (m, 1H), 3.75-3.61 (m, 1H), 3.48-3.12 (m, 5H), 3.11-3.00 (m, 2H), 2.98-2.86 (m, 2H), 2.47-2.27 (m, 3H), 2.12-1.98 (m, 2H), 1.67-1.57 (m, 1H), 1.33-1.23 (m, 6H) ppm.

### (9H-fluoren-9-yl)methyl (4-fluorophenethyl)(3-hydroxypropyl)carbamate (571)

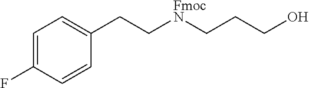

640 mg (3.24 mmol; 1.00 eq) of 3-((4-fluorophenethyl)amino)propan-1-ol were dissolved in a mixture of 30 mL Dioxane and 15 mL of a 10% NaHCO₃ solution. The solution was cooled to 0° C. with an ice-bath and 922 mg (3.56 mmol; 1.10 eq) of 9-Fluorenylmethoxycarbonyl chloride were added portionwise. The reaction was warmed to RT and consequently stirred for 18 h. Afterwards 100 mL (5×) of Dichloromethane were added and an extraction was performed. The organic phases were dried over MgSO₄, the solvent was removed under reduced pressure and the resulting residue was adsorbed on silica gel. Column chromatography (Cyclohexane/EtOAc 100:0% to 50:50%) gave 1359 mg (3.24 mmol; quantitative) of the title compound in the form of a colorless oil. TLC: R_{f}=0.29 (Cyclohexane/EtOAc 1:1).

¹H-NMR: (DMSO-d₆, 400 MHZ) δ=7.86-7.79 (m, 2H), 7.61-7.54 (m, 2H), 7.37-7.31 (m, 2H), 7.30-7.25 (m, 2H), 7.15-7.09 (m, 1H), 7.07-6.94 (m, 2H), 6.81-6.73 (m, 1H), 4.56-4.47 (m, 1H), 4.39-4.32 (m, 1H), 4.30 (t, J=5.0 Hz, 1H), 4.21-4.16 (m, 1H), 3.25-3.18 (m, 2H), 3.16-3.08 (m, 1H), 3.03-2.95 (m, 1H), 2.92-2.81 (m, 2H), 2.66-2.58 (m, 1H), 2.28-2.17 (m, 1H), 1.46-1.35 (m, 1H), 1.28-1.18 (m, 1H) ppm.

### (9H-fluoren-9-yl)methyl (4-fluorophenethyl)(3-oxopropyl)carbamate (572)

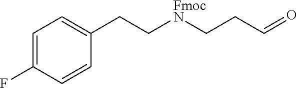

1359 mg (3.24 mmol; 1.00 eq) of (9H-fluoren-9-yl)methyl (4-fluorophenethyl)(3-hydroxypropyl)carbamate were dissolved in 50 ml of DCM. At 0° C., 1645 mg (3.89 mmol; 1.20 eq) of Dess-Martin-periodinane were added in portions. The reaction solution was continuously stirred for 1.5 h and thereby slowly warmed to RT. Then, 40 ml of a saturated NaHCO₃ solution were added. The organic phase was separated and extracted another 3-times with 80 mL of DCM. The organic phases were collected, dried over MgSO₄, filtered, and the solvent was removed under reduced pressure. Column chromatography on silica gel (Cyclohexane/EtOAc 100:0% to 65:35%) gave 1270 mg (3.04 mmol; 94%) of the title compound in the form of a colorless oil. TLC: R_{f}=0.63 (Cyclohexane/EtOAc 1:1). ¹H-NMR: (DMSO-d₆, 400 MHZ) δ=9.58 (s, 1H), 7.95-7.82 (m, 2H), 7.70-7.59 (m, 2H), 7.45-7.39 (m, 2H), 7.37-7.32 (m, 2H), 7.20-7.13 (m, 1H), 7.10-7.03 (m, 2H), 6.91-6.82 (m, 1H), 4.62-4.56 (m, 1H), 4.54-4.47 (m, 1H), 4.31-4.24 (m, 1H), 3.34-3.24 (m, 2H), 3.10-3.01 (m, 1H), 2.99-2.92 (m, 1H), 2.70-2.60 (m, 1H), 2.57-2.52 (m, 1H), 2.34-2.26 (m, 1H), 2.23-2.15 (m, 1H) ppm.

### (9//-fluoren-9-yl)methyl (3-((((3aR,4R,6R,6as)-6-(4-amino-2-chloro-5-phenyl-7H pyrrolo[2,3-d]pyrimidin-7-yl)-2,2-dimethyltetrahydro-4H-cyclopenta[d][1,3]dioxol-4-yl)methyl)amino)propyl)(4-fluorophenethyl)carbamate (573)

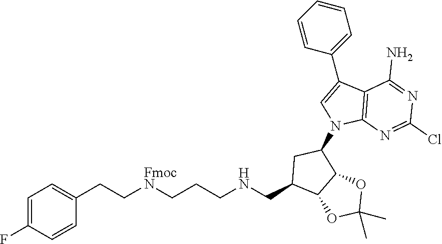

150 mg (0.360 mmol; 0.60 eq) of (9H-fluoren-9-yl)methyl (4-fluorophenethyl)(3-oxopropyl)carbamate were dissolved in 15 ml of dry DCE. 248 mg of 7-((3as,4R,6R,6aR)-6-(aminomethyl)-2,2-dimethyltetrahydro-4H-cyclopenta[d][1,3]dioxol-4-yl)-2-chloro-5-phenyl-7H pyrrolo[2,3-d]pyrimidin-4-amine (0.600 mmol; 1.00 eq) and 190 mg of NaB (OAc) 3H (0.900 mmol; 1.50 eq) were added. The reaction mixture was stirred for 18 h at RT. Afterwards the crude was purified by flash chromatography eluting with DCM/MeOH (1.:100:0% to 90:10%; 2.:100:0% to 80:20%). The title compound was yielded in form of a yellow foam (140 mg; 0.172 mmol; 48%). TLC: R_{f}=0.13 (DCM/MeOH 20:1). APCI-MS (+) m/z for C₄₇H₄₈ClFN₆O₄: calc.: 815.39; found: 592.6, 593.8 and 594.6 (without Fmoc-protection).

### (9//-fluoren-9-yl)methyl (3-((((3aR,4R,6R,6as)-6-(4-amino-2-chloro-5-phenyl-7H pyrrolo[2,3-d]pyrimidin-7-yl)-2,2-dimethyltetrahydro-4H-cyclopenta[d][1,3]dioxol-4-yl)methyl)(methyl)amino)propyl)(4-fluorophenethyl)carbamate (574)

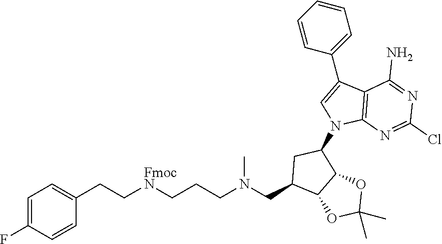

140 mg (0.170 mmol; 1.00 eq) of (9H-fluoren-9-yl)methyl (3-((((3aR,4R,6R,6as)-6-(4-amino-2-chloro-5-phenyl-7H-pyrrolo[2,3-d]pyrimidin-7-yl)-2,2-dimethyltetrahydro-4H-cyclopenta[d][1,3]dioxol-4-yl)methyl)amino)propyl)(4-fluorophenethyl)carbamate were dissolved in 15 mL of DCE and reacted at RT under N₂ atmosphere with 46 μL (1.70 mmol; 10.0 eq) of a 37% Formaldehyde solution in H₂O as well as 540 mg (2.55 mmol; 15.0 eq) of NaB (OAc) 3H. After 15 h of stirring the solvent was removed under reduced pressure and the resulting residue was adsorbed on silica gel. Column chromatography (DCM/MeOH 100:0% to 80:20%) gave 141 mg (0.170 mmol; quantitative) of the title compound in the form of a yellow solid. TLC: R_{f}=0.47 (DCM/MeOH 10:1). APCI-MS (+) m/z for C₄₈H₅₀ClFN₆₀₄: calc.: 829.41; found: 606.6, 607.7 and 608.6 (without Fmoc-protection).

### (1R,2S,3R,5R)-3-(4-amino-2-chloro-5-phenyl-7H-pyrrolo[2,3-d]pyrimidin-7-yl)-5-(((3-((4-fluorophenethyl)amino)propyl)(methyl)amino)methyl)cyclopentane-1,2-diol (575)

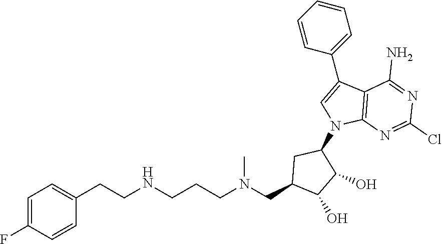 [SMILES: CN(CCCNCCc1ccc(F)cc1)C[C@H]1C[C@@H](n2cc(-c3ccccc3)c3c(N)nc(Cl)nc32)[C@H](O)[C@@H]1O]

141 mg (0.170 mmol; 1.00 eq) of (9H-fluoren-9-yl)methyl (3-((((3aR,4R,6R,6as)-6-(4-amino-2-chloro-5-phenyl-7H-pyrrolo[2,3-d]pyrimidin-7-yl)-2,2-dimethyltetrahydro-4H-cyclopenta[d][1,3]dioxol-4-yl)methyl)(methyl)amino)propyl)(4-fluorophenethyl)carbamate were dissolved in a mixture of 3.0 ml of H₂O and 3.0 mL of TFA and the reaction mixture was subsequently stirred for 5 h. After removing the solvent under reduced pressure, the remaining residue was again dissolved in 3.0 ml of H₂O and 3.0 ml of Piperidine and stirred for 10 h. The resulting product was purified via preparative HPLC according to procedure A (MeCN/H₂O+0.1% TFA; 10/90% to 100/0%; t_{R}=13.90 min; UV-purity at 210, 230, 254, 242 and 275 nm >99%) to give the title compound (45 mg; 0.057 mmol; 34%) in form of a white foam and as 2-fold TFA salt. APCI-MS (+) m/z for C₃₀H₃₆ClFN₆O₂: calc.: 566.26; found: 566.8, 567.8 and 568.7. ¹H-NMR: (DMSO-d₆, 400 MHZ) δ=9.55 (bs, 1H), 8.79 (s, 2H), 7.53-7.43 (m, 5H), 7.40-7.35 (m, 1H), 7.34-7.28 (m, 2H), 7.21-7.15 (m, 2H), 4.94-4.84 (m, 1H), 4.19 (t, J=6.4 Hz, 1H), 3.87 (t, J=5.5 Hz, 1H), 3.43-2.96 (m, 8H), 2.94-2.88 (m, 2H), 2.86-2.80 (m, 3H), 2.44-2.34 (m, 2H) 2.09-1.94 (m, 2H), 1.68-1.56 (m, 1H) ppm.

### 2-chloro-5-iodo-7H-pyrrolo[2,3-d]pyrimidine (576)

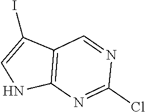 [SMILES: Clc1ncc2c(I)c[nH]c2n1]

1536 mg (10.00 mmol; 1.00 eq) of 2-chloro-7H-pyrrolo[2,3-d]pyrimidine were dissolved in 40 ml of DMF. 2475 (11.00 mmol; 1.10 eq) of N-lodsuccinimid were added and after 16 h of stirring at RT, the reaction mixture was quenched with 100 ml of a solution of saturated NaHCO₃ and then extracted with EtOAc (6×50 mL). The organic phases were combined, dried with MgSO₄, and then purified by flash chromatography (Cyclohexane/EtOAc 99:1% to 65:35%) to obtain 1786 mg (7.68 mmol; 77%) of the title compound in form of a beige solid. TLC: R_{f}=0.42 (Cyclohexane/EtOAc 3:1). APCI-MS (+) m/z for C₆H₃ClIN3: calc.: 279.47; found: 279.9 and 280.9. ¹H-NMR: (DMSO-d₆, 400 MHZ) δ=12.77 (s, 1H); 8.67 (s, 1H), 7.88 (s, 1H) ppm. 7-((3as,4R,6R,6aR)-6-(((tert-butyldimethylsilyl)oxy)methyl)-2,2-dimethyltetrahydro-4H-cyclopenta[d][1,3]dioxol-4-yl)-2-chloro-5-iodo-7H-pyrrolo[2,3-d]pyrimidine (577)

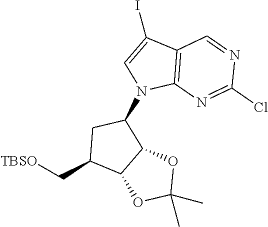 [SMILES: CC1(C)O[C@@H]2[C@@H](CO[Si](C)(C)C(C)(C)C)C[C@@H](n3cc(I)c4cnc(Cl)nc43)[C@@H]2O1]

1466 mg (4.85 mmol; 1.00 eq) of (3as,45,6R,6aR)-6-(((tert-butyldimethylsilyl)oxy)methyl)-2,2-dimethyltetrahydro-4H-cyclopenta[d][1,3]dioxol-4-ol were dissolved in 50 ml of Toluene and then cooled with an ice-bath to 0° C. 2544 mg (9.70 mmol; 2.00 eq) of PPh₃ were given to the reaction mixture. After 5 minutes 2234 mg (9.70 mmol; 2.00 eq) of DBAD were added. After 10 more minutes 1355 mg (4.85 mmol; 1.00 eq) of 2-chloro-5-iodo-7H-pyrrolo[2,3-d]pyrimidine were added. The reaction mixture was then heated to 80° C. under reflux overnight and thereby continuously stirred. The crude was purified by flash chromatography (Cyclohexane/EtOAc (99:1% to 85:15%) to obtain 2200 mg (3.90 mmol; 80%) of the title compound in form of a purple foam. TLC: R_{f}=0.66 (Cyclohexane/EtOAc 3:1). APCI-MS (+) m/z for C₂₁H₃₁ClIN₃O₃Si: calc.: 563.94; found: 563.5 and 565.4. ¹H-NMR: (DMSO-d₆, 400 MHZ) δ=8.68 (s, 1H), 8.09 (s, 1H), 5.03-4.97 (m, 1H); 4.89-4.86 (m, 1H), 4.51-4.48 (m, 1H), 3.73-3.63 (m, 2H), 2.35-2.26 (m, 2H), 2.10-2.06 (m, 1H), 1.48 (s, 3H), 1.21 (s, 3H), 0.87 (s, 9H), 0.06 (s, 6H) ppm.

### 7-((3as,4R,6R,6aR)-6-(((tert-butyldimethylsilyl)oxy)methyl)-2,2-dimethyltetrahydro-4H-cyclopenta[d][1,3]dioxol-4-yl)-2-chloro-5-(thiophen-2-yl)-7//pyrrolo[2,3-d]pyrimidine (578)

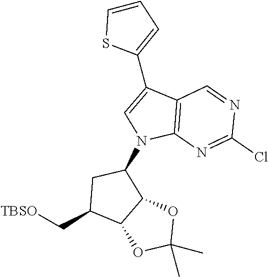 [SMILES: CC1(C)O[C@@H]2[C@@H](CO[Si](C)(C)C(C)(C)C)C[C@@H](n3cc(-c4cccs4)c4cnc(Cl)nc43)[C@@H]2O1]

7-((3as,4R,6R,6aR)-6-(((tert-butyldimethylsilyl)oxy)methyl)-2,2-dimethyltetrahydro-4H cyclopenta[d][1,3]dioxol-4-yl)-2-chloro-5-iodo-7H-pyrrolo[2,3-d]pyrimidine (1100 mg; 1.95 mmol; 1.00 eq), K₃PO₄ (828 mg; 3.90 mmol; 2.00 eq), and Thiophene-2-boronic acid pinacol ester (704 mg; 3.35 mmol; 1.72 eq) were dissolved in a mixture of 10.0 ml of Dioxane and 10.0 mL of degassed H₂O. Then the catalyst Pd (dtbpf) Cl₂ (0.10 eq) was given to the reaction mixture and afterwards, it was heated to 90° C. under reflux with stirring. After 45 min the reaction mixture was quenched with 100 ml of a solution of saturated NaCl, extracted with EtOAc (5×50 mL), dried with MgSO₄, and finally purified by flash chromatography eluting with Cyclohexane/EtOAc (99:1% to 75:25%) to obtain the title compound (581 mg; 1.12 mmol; 57%) in form of a brown oil. TLC: R$ =0.38 (Cyclohexane/EtOAc 3:1). APCI-MS (+) m/z for C₂₅H₃₄ClN₃O₃SSi: calc.: 519.2; found 519.6 and 521.5. ¹H-NMR: (DMSO-d₆, 400 MHZ) δ=9.24 (s, 1H), 8.20 (s, 1H), 7.56-7.53 (m, 2H), 7.19-7.15 (m, 1H), 5.09-5.00 (m, 1H), 4.97-4.93 (m, 1H), 4.56-4.50 (m, 1H), 3.76-3.66 (m, 2H), 2.33-2.25 (m, 2H), 2.18-2.07 (m, 1H), 1.50 (s, 3H), 1.23 (s, 3H), 0.88 (s, 9H), 0.07 (s, 6H) ppm.

### ((3aR,4R,6R,6as)-6-(2-chloro-5-(thiophen-2-yl)-7H-pyrrolo[2,3-d]pyrimidin-7-yl)-2,2-dimethyltetrahydro-4H-cyclopenta[d][1.3]dioxol-4-yl)methanol (579)

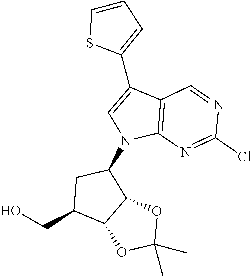 [SMILES: CC1(C)O[C@@H]2[C@@H](CO)C[C@@H](n3cc(-c4cccs4)c4cnc(Cl)nc43)[C@@H]2O1]

640 mg (1.23 mmol; 1.00 eq) of 7-((3as,4R,6R,6aR)-6-(((tert-butyldimethylsilyl)oxy)methyl)-2,2-dimethyltetrahydro-4H-cyclopenta[d][1,3]dioxol-4-yl)-2-chloro-5-(thiophen-2-yl)-7H-pyrrolo[2,3-d]pyrimidine were dissolved in 10 ml of THF and 1.85 ml (1.85 mmol; 1.50 eq) of a 1 M solution of TBAF in THF were added. The reaction mixture was stirred overnight and then the solvent was removed under reduced pressure. Column chromatography on silica gel (Cyclohexane/EtOAc 100:0% to 40:60%) gave 415 mg (1.02 mmol; 83%) of the title compound in the form of a colorless foam. TLC: R_{f}=0.34 (Cyclohexane/EtOAc 1:1). APCI-MS (+) m/z for C₁₉H₂₀ClN₃O₃S: calc.: 405.90; found: 405.5 and 407.5. ¹H-NMR: (DMSO-d₆, 400 MHZ) δ=9.26 (s, 1H), 8.30 (s, 1H), 7.59-7.55 (m, 2H), 7.19 (dd, J=5.1, 3.6 Hz, 1H), 5.11-5.01 (m, 1H), 4.94-4.88 (m, 1H), 4.81 (t, J=5.3 Hz, 1H), 4.57-4.52 (m, 1H), 3.57-3.49 (m, 2H), 2.35-2.22 (m, 2H), 2.15-2.03 (m, 1H), 1.52 (s, 3H), 1.25 (s, 3H) ppm.

### (3aR,6R,6as)-6-(2-chloro-5-(thiophen-2-yl)-7H-pyrrolo[2,3-d]pyrimidin-7-yl)-2,2-dimethyltetrahydro-4H-cyclopenta[d][1,3]dioxole-4-carbaldehyde and tert-butyl (3-((((3aR,4R,6R,6as)-6-(2-chloro-5-(thiophen-2-yl)-7H-pyrrolo[2,3-d]pyrimidin-7-yl)-2,2-dimethyltetrahydro-4H-cyclopenta[d][1,3]dioxol-4-yl)methyl)amino)propyl)(4-fluorophenethyl)carbamate (580)

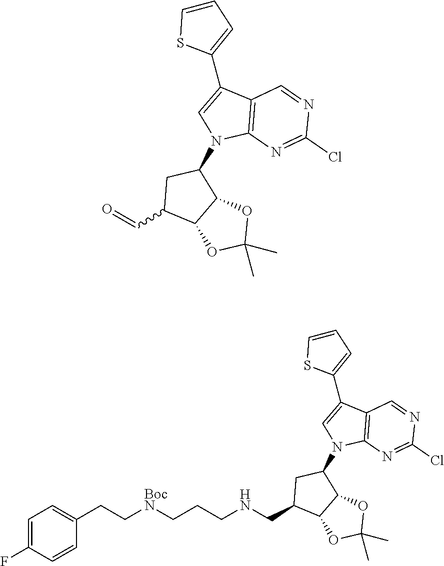 [SMILES: CC1(C)O[C@@H]2[C@H](O1)C(C=O)C[C@H]2n1cc(-c2cccs2)c2cnc(Cl)nc21 ; CC(C)(C)OC(=O)N(CCCNC[C@H]1C[C@@H](n2cc(-c3cccs3)c3cnc(Cl)nc32)[C@@H]2OC(C)(C)O[C@H]12)CCc1ccc(F)cc1]

((3aR,4R,6R,6as)-6-(2-chloro-5-(thiophen-2-yl)-7H-pyrrolo[2,3-d]pyrimidin-7-yl)-2,2-dimethyltetrahydro-4H-cyclopenta[d][1,3]dioxol-4-yl)methanol (711 mg; 1.75 mmol; 1.00 eq) was dissolved in 40 ml of DCM. After cooling with an ice-bath to 0° C., Dess-Martin-periodinane (965 mg; 2.28 mmol; 1.30 eq) was added. 3 h later, when TLC showed no more educt, the reaction mixture was warmed to RT and then quenched with 100 ml of a solution of saturated NaHCO₃ and extracted five times with 50 mL of DCM. Afterwards, the organic phases were combined, dried with MgSO₄ and the solvent was removed under reduced pressure. The remaining residue was purified by flash chromatography eluting with Petrolether/EtOAc (99%/1% to 60%/40%; R_{f}=0.55 (Cyclohexane/EtOAc 1:1) to obtain the desired product, which was used without further characterization. For further reaction 130 mg (0.320 mmol; 1.00 eq) of (3aR,6R,6as)-6-(2-chloro-5-(thiophen-2-yl)-7H-pyrrolo[2,3-d]pyrimidin-7-yl)-2,2-dimethyltetrahydro-4H-cyclopenta[d][1,3]dioxole-4-carbaldehyde were dissolved in 15 ml of DCE and reacted at RT under N₂ atmosphere with 94 mg (0.320 mmol; 1.00 eq) of tert-butyl (4-fluorophenethyl)(3-oxopropyl)carbamate and 102 mg (0.480 mmol; 1.50 eq) of NaB (OAc) 3H. After 20 h of stirring the solvent was removed under reduced pressure and the resulting residue was adsorbed on silica gel. Column chromatography (DCM/MeOH 100:0% to 90:10%) gave 170 mg (0.248 mmol; 77%) of the title compound in the form of a colorless foam. TLC: R_{f}=0.54 (DCM/MeOH 10:1). APCI-MS (+) m/z for C₃₅H₄₃ClFN₅O₄S: calc.: 684.27; found: 684.0 and 685.9.

### (1R,2S,3R)-3-(2-chloro-5-(thiophen-2-yl)-7H pyrrolo[2,3-d]pyrimidin-7-yl)-5-(((3-((4-fluorophenethyl)amino)propyl)amino)methyl)cyclopentane-1,2-diol (581)

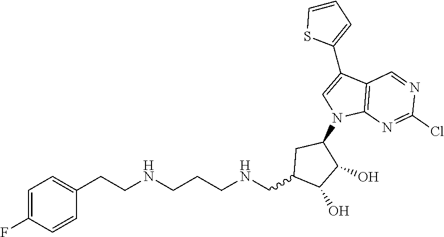 [SMILES: O[C@@H]1[C@H](O)C(CNCCCNCCc2ccc(F)cc2)C[C@H]1n1cc(-c2cccs2)c2cnc(Cl)nc21]

170 mg (0.248 mmol; 1.00 eq) of tert-butyl (3-((((3aR,4R,6R,6as)-6-(2-chloro-5-(thiophen-2-yl)-7H-pyrrolo[2,3-d]pyrimidin-7-yl)-2,2-dimethyltetrahydro-4H-cyclopenta[d][1,3]dioxol-4-yl)methyl)amino)propyl)(4-fluorophenethyl)carbamate were dissolved in a mixture of 3.0 ml of H₂O and 3.0 ml of TFA. The reaction mixture was subsequently stirred overnight. Then the solvent was removed under reduced pressure. The resulting product was purified via preparative HPLC according to procedure B (MeCN/H₂O+0.1% TFA; 10/90% to 100/0%; t_{R}=10.77 min; UV-purity at 210, 230, 254, 242 and 275 nm >98%) to give the title compound (34 mg; 0.044 mmol; 18%) in form of a white foam (as 2-fold TFA salt and mixture of diastereomers in a ratio of 1:1.4). APCI-MS (+) m/z for C₂₇H₃₁ClFN₅O₂S: calc.: 543.19; found: 544.1, 545.1 and 546.1. ¹H-NMR: (DMSO-d₆, 400 MHZ) δ=9.25 and 9.24 (s, 2H), 8.80 (bs, 4H), 8.66 (bs, 2H), 8.55 (bs, 2H), 8.25 and 8.21 (s, 2H), 7.57-7.53 (m, 4H), 7.34-7.29 (m, 4H), 7.21-7.15 (m, 6H), 5.12-5.06 and 5.03-4.97 (m, 2H), 4.58-4.52 and 4.32-4.28 (m, 2H), 4.07-4.03 and 3.93-3.89 (m, 2H), 3.27-3.11 (m, 7H), 3.09-2.96 (m, 10H), 2.95-2.87 (m, 4H), 2.42-2.34 (m, 1H), 2.16-2.04 (m, 2H), 2.03-1.91 (m, 5H), 1.73-1.65 (m, 1H) ppm.

### Synthesis of (1R,2S,3R,5R)-3-{4-amino-2-chloropyrrolo[2,3-d]pyrimidin-7-yl}-5-(1H-pyrazol-4-yl)cyclopentane-1,2-diol (582)

### (3aR,6R,6aR)-2,2-Dimethyl-6-[1-(oxan-2-yl)pyrazol-4-yl]-tetrahydrocyclopenta[d][1,3]dioxol-4-one (142b)

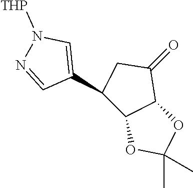

Into a 40 mL vial were added (3aR,6aR)-2,2-dimethyl-3aH,6aH-cyclopenta[d][1,3]dioxol-4-one (0.5 g, 3.2 mmol, 1.0 eq), 1-(oxan-2-yl)-4-(4,4,5,5-tetramethyl-1,3,2-dioxaborolan-2-yl)pyrazole (1.35 g, 4.9 mmol, 1.5 eq), acetylacetonato bis(ethylene) rhodium (I)(83.7 mg, 0.32 mmol, 0.1 eq) and R-BINAP (260 mg, 0.32 mmol, 0.1 eq) in dioxane (5 mL) at room temperature. The reaction was stirred for 2 h at 100° C., then concentrated. The residue was dissolved in DCM (3 mL) and was purified by silica gel column chromatography (2:1 Petroleum ether: AcOEt) to afford (3aR,6R,6aR)-2,2-dimethyl-6-[1-(oxan-2-yl)pyrazol-4-yl]-tetrahydrocyclopenta[d][1,3]dioxol-4-one 142b (0.5 g, 55%) as a white solid. LCMS (conditions: XBridge C18, 50*3.0 mm, 3.5 μm, Mobile Phase A: Water/5 mM NH₄HCO₃, Mobile Phase B: Methanol, 10%-95% B-2 min, 1.50 mL/min): T_{R}=1.28 min; ES m/z [M+H]⁺: 307.

### (3aS,4S,6R,6aR)-2,2-Dimethyl-6-[1-(oxan-2-yl)pyrazol-4-yl]-tetrahydro-3aH-cyclopenta[d][1,3]dioxol-4-ol (143b)

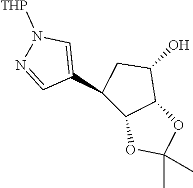

Into a 40 mL vial were added (3aR,6R,6aR)-2,2-dimethyl-6-[1-(oxan-2-yl)pyrazol-4-yl]-tetrahydrocyclopenta[d][1,3]dioxol-4-one 142b (0.5 g, 1.8 mmol, 1.0 eq) in MeOH (5 mL), followed by NaBH₄ (0.17 g, 2.7 mmol, 1.5 eq) at 0° C. The reaction was stirred for 0.5 h at room temperature, then concentrated. The residue was dissolved in DCM (3 mL) and was purified by silica gel column chromatography (3:2 Petroleum ether: AcOEt) to afford (3aS,4S,6R,6aR)-2,2-dimethyl-6-[1-(oxan-2-yl)pyrazol-4-yl]-tetrahydro-3aH-cyclopenta[d][1,3]dioxol-4-ol 143b (0.4 g, 79%) as a white solid. LCMS (conditions Xbridge Shield C18, 50*3.0 mm, 3.5 μm, Mobile Phase A: Water/0.05% Ammonia, Mobile Phase B: Acetonitrile, 5-100% B-2 min, 1.50 mL/min): T_{R}=0.89 min; ES m/z [M+H]⁺: 309.

### 4-[(3aR,4R,6R,6aS)-6-{2,4-Dichloropyrrolo[2,3-d]pyrimidin-7-yl}-2,2-dimethyl-tetrahydro-3aH-cyclopenta[d][1,3]dioxol-4-yl]-1-(oxan-2-yl)pyrazole (144b)

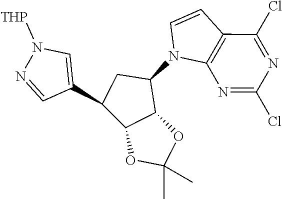

Into a 40 mL vial were added (3aS,4S,6R,6aR)-2,2-dimethyl-6-[1-(oxan-2-yl)pyrazol-4-yl]-tetrahydro-3aH-cyclopenta[d][1,3]dioxol-4-ol 143b (0.2 g, 0.65 mmol, 1.2 eq), 2,4-dichloro-7H-pyrrolo[2,3-d]pyrimidine (0.1 g, 0.54 mmol, 1.0 eq), PPh₃ (0.28 g, 1.1 mmol, 2.0 eq) and THF (5 mL), followed by DIAD (0.2 g, 1.1 mmol, 2.0 eq) at 0° C. The resulting mixture was stirred for 12 h at room temperature, then concentrated. The residue was dissolved in DCM (3 mL) and was purified by silica gel column chromatography (1:1 Petroleum ether: AcOEt) to afford 4-[(3aR,4R,6R, 6aS)-6-{2,4-dichloropyrrolo[2,3-d]pyrimidin-7-yl}-2,2-dimethyl-tetrahydro-3aH-cyclopenta[d][1,3]dioxol-4-yl]-1-(oxan-2-yl)pyrazole 144b (0.18 g, 71%) as a white solid. LCMS (conditions XBridge C18, 50*3.0 mm, 3.5 μm, Mobile Phase A: Water/5 mM NH₄HCO₃, Mobile Phase B: Acetonitrile, 5%-100% B-2 min, 1.50 mL/min,): T_{R}=1.28 min; ES m/z [M+H]⁺: 478, 480.

### 7-[(3aS,4R,6R,6aR)-2,2-Dimethyl-6-[1-(oxan-2-yl)pyrazol-4-yl]-tetrahydro-3aH-cyclopenta[d][1,3]dioxol-4-yl]-2-chloropyrrolo[2,3-d]pyrimidin-4-amine (145b)

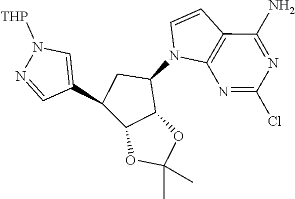

Into a 40 mL vial were added 4-[(3aR,4R,6R,6aS)-6-{2,4-dichloropyrrolo[2,3-d]pyrimidin-7-yl}-2,2-dimethyl-tetrahydro-3aH-cyclopenta[d][1,3]dioxol-4-yl]-1-(oxan-2-yl)pyrazole 144b (91 mg, 0.19 mmol, 1.0 eq) and 7M NH₃ in MeOH (2 mL) at room temperature. The resulting mixture was stirred for 8 h at 80° C. The resulting mixture was concentrated to afford 7-[(3aS,4R,6R,6aR)-2,2-dimethyl-6-[1-(oxan-2-yl)pyrazol-4-yl]-tetrahydro-3aH-cyclopenta[d][1,3]dioxol-4-yl]-2-chloropyrrolo[2,3-d]pyrimidin-4-amine 145b (60 mg, crude) as a yellow solid. LCMS (conditions XBridge C18, 50*3.0 mm, 3.5 μm, Mobile Phase A: Water/5 mM NH₄HCO₃, Mobile Phase B: Acetonitrile, 5%-100% B-2 min, 1.50 mL/min,): T_{R}=1.34 min; ES m/z [M+H]⁺: 459, 461.

### (1R,2S,3R,5R)-3-{4-Amino-2-chloropyrrolo[2,3-d]pyrimidin-7-yl}-5-(1H-pyrazol-4-yl)cyclopentane-1,2-diol (582)

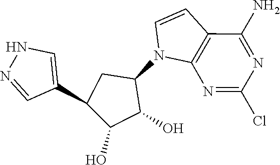 [SMILES: Nc1nc(Cl)nc2c1ccn2[C@@H]1C[C@H](c2cn[nH]c2)[C@@H](O)[C@H]1O]

Into an 8 mL vial were added 7-[(3aS,4R,6R,6aR)-2,2-dimethyl-6-[1-(oxan-2-yl)pyrazol-4-yl]-tetrahydro-3aH-cyclopenta[d][1,3]dioxol-4-yl]-2-chloropyrrolo[2,3-d]pyrimidin-4-amine 145b (60 mg, 0.13 mmol, 1.0 eq) and 4M HCl in MeOH (1.0 mL) at room temperature. The resulting mixture was stirred for 0.5 h at 50° C., then concentrated. The residue was dissolved in MeOH (3 mL) and basified to pH>8 with NH₃·H₂O. The mixture was purified by Prep-HPLC [column, Xbridge Prep C18 OBD column, 5 μm, 19*150 mm; mobile phase, Water (0.03% NH₄OH) and CH₃CN (15% to 50% CH₃CN over 10 min); Detector, UV 220&254 nm]to afford (1R,2S,3R,5R)-3-{4-amino-2-chloropyrrolo[2,3-d]pyrimidin-7-yl}-5-(1H-pyrazol-4-yl)cyclopentane-1,2-diol 582 (10.1 mg, 23%) as a white solid. LCMS (conditions: ZORBAX SB-Aq, 50*4.6 mm, 1.8 μm, Mobile Phase A: Water/0.02% TFA, Mobile Phase B: Acetonitrile, 5%-95% B-3 min, 1.50 mL/min): T_{R}=1.17 min; ES m/z [M+H]⁺: 335, 337. ¹H NMR (400 MHZ, DMSO-d₆) δ 12.59 (br, s, 1H), 7.74-7.37 (m, 4H), 7.30 (d, J=3.5 Hz, 1H), 6.58 (d, J=3.5 Hz, 1H), 4.90-4.86 (m, 2H), 4.86-4.76 (m, 1H), 4.21 (q, J=6.3 Hz, 1H), 3.88 (q, J=5.8 Hz, 1H), 3.08-2.97 (m, 1H), 2.47-2.36 (m, 1H), 1.91-1.78 (m, 1H).

### Synthesis of (1R,2S,3R,5R)-3-[2-chloro-4-(methylamino)pyrrolo[2,3-d]pyrimidin-7-yl]-5-(1H-pyrazol-4-yl)cyclopentane-1,2-diol (583)

### 7-[(3aS,4R,6R,6aR)-2,2-Dimethyl-6-[1-(oxan-2-yl)pyrazol-4-yl]-tetrahydro-3aH-cyclopenta[d][1,3]dioxol-4-yl]-2-chloro-N-methylpyrrolo[2,3-d]pyrimidin-4-amine (149b)

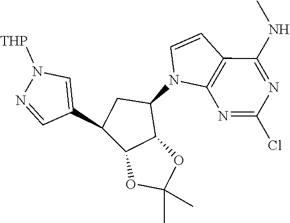

Into an 8 mL vial were added 4-[(3aR,4R,6R,6aS)-6-{2,4-dichloropyrrolo[2,3-d]pyrimidin-7-yl}-2,2-dimethyl-tetrahydro-3aH-cyclopenta[d][1,3]dioxol-4-yl]-1-(oxan-2-yl)pyrazole 144b (91 mg, 0.19 mmol, 1.0 eq) and 2M methylamine in methanol (2 mL) at room temperature. The reaction was stirred for 1 h at 80° C. The resulting mixture was concentrated to afford 7-[(3aS,4R,6R,6aR)-2,2-dimethyl-6-[1-(oxan-2-yl)pyrazol-4-yl]-tetrahydro-3aH-cyclopenta[d][1,3]dioxol-4-yl]-2-chloro-N-methylpyrrolo[2,3-d]pyrimidin-4-amine 149b (80 mg, 89%) as a light yellow solid. LCMS (conditions XBridge C18, 50*3.0 mm, 3.5 μm, Mobile Phase A: Water/5 mM NH₄HCO₃, Mobile Phase B: Acetonitrile, 5%-100% B-2 min, 1.50 mL/min,): T_{R}=1.42 min; ES m/z [M+H]⁺: 473, 475.

### (1R,2S,3R,5R)-3-[2-chloro-4-(methylamino)pyrrolo[2,3-d]pyrimidin-7-yl]-5-(1H-pyrazol-4-yl)cyclopentane-1,2-diol (583)

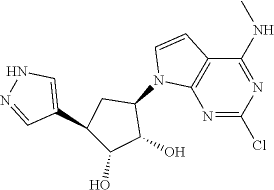 [SMILES: CNc1nc(Cl)nc2c1ccn2[C@@H]1C[C@H](c2cn[nH]c2)[C@@H](O)[C@H]1O]

Into an 8 mL vial were added 7-[(3aS,4R,6R,6aR)-2,2-dimethyl-6-[1-(oxan-2-yl)pyrazol-4-yl]-tetrahydro-3aH-cyclopenta[d][1,3]dioxol-4-yl]-2-chloro-N-methylpyrrolo[2,3-d]pyrimidin-4-amine 149b (80 mg, 0.17 mmol, 1.0 eq) and 4M HCl in MeOH (2 mL) at room temperature. The resulting mixture was stirred for 0.5 h at 50° C., then concentrated. The residue was dissolved in MeOH (3 mL) and basified to pH>8 with NH₃·H₂O. The mixture was purified by Prep-HPLC [column, Xbridge Prep C18 OBD column, 5 μm, 19*150 mm; mobile phase, Water (0.03% NH₄OH) and CH₃CN (15-45% over 10 min); Detector, UV 220&254 nm]to afford (1R,2S,3R,5R)-3-[2-chloro-4-(methylamino)pyrrolo[2,3-d]pyrimidin-7-yl]-5-(1H-pyrazol-4-yl)cyclopentane-1,2-diol 583 (14.1 mg, 24%) as a white solid. LCMS (conditions: ZORBAX SB-Aq, 50*4.6 mm, 1.8 μm, Mobile Phase A: Water/0.02% TFA, Mobile Phase B: Acetonitrile, 5%-95% B-3 min, 1.50 mL/min): T_{R}=1.30 min; ES m/z [M+H]⁺: 349, 351.1H NMR (300 MHZ, DMSO-d₆) δ 12.58 (br, s, 1H), 7.89 (d, J=4.4 Hz, 1H), 7.65-7.49 (m, 2H), 7.30 (d, J=3.6 Hz, 1H), 6.58 (d, J=3.5 Hz, 1H), 4.88-4.75 (m, 3H), 4.21 (q, J=6.3 Hz, 1H), 3.88 (q, J=5.8 Hz, 1H), 3.09-2.90 (m, 4H), 2.48-2.34 (m, 1H), 1.94-1.77 (m, 1H).

### Synthesis of (1R,2S,3R,5R)-3-(4-amino-2-chloro-7H-pyrrolo[2,3-d]pyrimidin-7-yl)-5-(1-phenethyl-1H-pyrazol-4-yl)cyclopentane-1,2-diol (584)

### 2,4-Dichloro-7-((3aS,4R,6R,6aR)-2,2-dimethyl-6-(1H-pyrazol-4-yl)tetrahydro-4H-cyclopenta[d][1,3]dioxol-4-yl)-7H-pyrrolo[2,3-d]pyrimidine (146b)

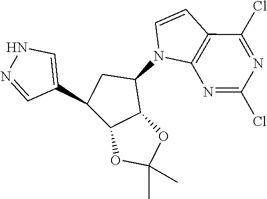 [SMILES: CC1(C)O[C@@H]2[C@H](O1)[C@@H](c1cn[nH]c1)C[C@H]2n1ccc2c(Cl)nc(Cl)nc21]

Into a 40 mL vial were added 2,4-dichloro-7-((3aS,4R,6R,6aR)-2,2-dimethyl-6-(1-(tetrahydro-2H-pyran-2-yl)-1H-pyrazol-4-yl)tetrahydro-4H-cyclopenta[d][1,3]dioxol-4-yl)-7H-pyrrolo[2,3-d]pyrimidine 144b (0.14 g, 0.3 mmol, 1.0 eq) and 4M HCl in MeOH (3 mL) at room temperature. The reaction mixture was stirred at 40° C. for 30 min, then concentrated. The residue was dissolved in acetone (3 mL), then 2,2-dimethoxypropane (1.0 mL, 0.028 mmol) and p-TsOH (1.94 mg, 0.011 mmol, 0.1 eq) were added. The reaction was stirred for 2 h, then concentrated. The residue was dissolved in CH₃CN (3 mL) and basified to pH>8 with NaHCO₃. The mixture was purified by reverse phase flash with the following conditions (Column: Welch Utimate AQ-C18, 50*250 mm*10 μm; Mobile Phase A: Water (10 mmol/L NH₄HCO₃+0.1% NH₃·H₂O), Mobile Phase B: ACETONITRILE; Flow rate: 80 mL/min; Gradient: 5% B to 60% B over 15 min, Wavelength: 220 nm) to afford 2,4-dichloro-7-((3aS,4R,6R,6aR)-2,2-dimethyl-6-(1H-pyrazol-4-yl)tetrahydro-4H-cyclopenta[d][1,3]dioxol-4-yl)-7H-pyrrolo[2,3-d]pyrimidine 146b (20 mg, 44%) as a white solid. LCMS (conditions Cortecs C18+, 50*3.0 mm, 2.7 μm, Mobile Phase A: Water/0.01% FA, Mobile Phase B: Acetonitrile/0.01% FA, 2%-100% B-2.5 min, 1.50 mL/min): T_{R}=1.23 min; ES m/z [M+H]⁺: 394, 396

### 2,4-Dichloro-7-((3aS,4R,6R,6aR)-2,2-dimethyl-6-(1-phenethyl-1H-pyrazol-4-yl)tetrahydro-4H-cyclopenta[d][1,3]dioxol-4-yl)-7H-pyrrolo[2,3-d]pyrimidine (147b)

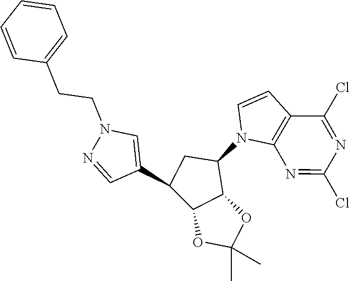 [SMILES: CC1(C)O[C@@H]2[C@H](O1)[C@@H](c1cnn(CCc3ccccc3)c1)C[C@H]2n1ccc2c(Cl)nc(Cl)nc21]

Into an 8 mL vial were added 2,4-dichloro-7-((3aS,4R,6R,6aR)-2,2-dimethyl-6-(1H-pyrazol-4-yl)tetrahydro-4H-cyclopenta[d][1,3]dioxol-4-yl)-7H-pyrrolo[2,3-d]pyrimidine 146b (30 mg, 0.076 mmol, 1.0 eq), (2-bromoethyl)benzene (18.3 mg, 0.099 mmol, 1.3 eq), K₂CO₃ (21.0 mg, 0.152 mmol, 2.0 eq) and DMAc (1.0 mL) at room temperature. The reaction was stirred at 80° C. for 2 h, cooled to room temperature, and filtered. The filtrate was purified by reverse phase flash chromatography with the following conditions (Column: Welch Utimate AQ-C18, 50*250 mm*10 μm; Mobile Phase A: Water (10 mmol/L NH₄HCO₃+0.1% NH₃·H₂O), Mobile Phase B: ACETONITRILE; Flow rate: 80 mL/min; Gradient: 5% B to 65% B over 15 min, Wavelength: 220 nm) to afford 2,4-dichloro-7-((3aS,4R,6R,6aR)-2,2-dimethyl-6-(1-phenethyl-1H-pyrazol-4-yl)tetrahydro-4H-cyclopenta[d][1,3]dioxol-4-yl)-7H-pyrrolo[2,3-d]pyrimidine 146b (10 mg, 26%) as a white solid. LCMS (conditions Proshell HPH-C18, 50*3.0 mm, 2.7 μm, Mobile Phase A: Water (0.05% ammonia water), Mobile Phase B: Acetonitrile, 2%-100% B-2.5 min, 1.50 mL/min): T_{R}=1.71 min; ES m/z [M+H]⁺: 498, 500.

### 2-Chloro-7-((3aS,4R,6R,6aR)-2,2-dimethyl-6-(1-phenethyl-1H-pyrazol-4-yl)tetrahydro-4H-cyclopenta[d][1,3]dioxol-4-yl)-7H-pyrrolo[2,3-d]pyrimidin-4-amine (147b)

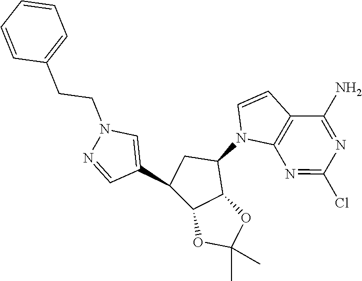 [SMILES: CC1(C)O[C@@H]2[C@H](O1)[C@@H](c1cnn(CCc3ccccc3)c1)C[C@H]2n1ccc2c(N)nc(Cl)nc21]

Into an 8 mL vial were added 2,4-dichloro-7-((3aS,4R,6R,6aR)-2,2-dimethyl-6-(1-phenethyl-1H-pyrazol-4-yl)tetrahydro-4H-cyclopenta[d][1,3]dioxol-4-yl)-7H-pyrrolo[2,3-d]pyrimidine 146b (10 mg, 0.02 mmol, 1.0 eq) and 7M NH₃ in MeOH (2 mL) at room temperature. The reaction was stirred at 80° C. for 2 h, cooled to room temperature, then concentrated. The crude product 2-chloro-7-((3aS,4R,6R,6aR)-2,2-dimethyl-6-(1-phenethyl-1H-pyrazol-4-yl)tetrahydro-4H-cyclopenta[d][1,3]dioxol-4-yl)-7H-pyrrolo[2,3-d]pyrimidin-4-amine 147b (6 mg, 62%) was used in the next step directly without further purification. LCMS (conditions Proshell HPH-C18, 50*3.0 mm, 2.7 μm, Mobile Phase A: Water (0.05% ammonia water), Mobile Phase B: Acetonitrile, 2%-100% B-2.5 min, 1.50 mL/min): T_{R}=1.37 min; ES m/z [M+H]⁺: 479, 481.

### (1R,2S,3R,5R)-3-(4-Amino-2-chloro-7H-pyrrolo[2,3-d]pyrimidin-7-yl)-5-(1-phenethyl-1H-pyrazol-4-yl)cyclopentane-1,2-diol (584)

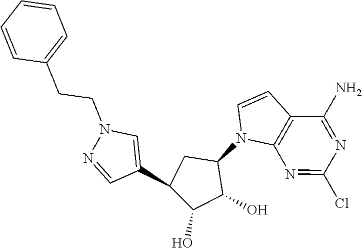 [SMILES: Nc1nc(Cl)nc2c1ccn2[C@@H]1C[C@H](c2cnn(CCc3ccccc3)c2)[C@@H](O)[C@H]1O]

Into an 8 mL vial were added 2-chloro-7-((3aS,4R,6R,6aR)-2,2-dimethyl-6-(1-phenethyl-1H-pyrazol-4-yl)tetrahydro-4H-cyclopenta[d][1,3]dioxol-4-yl)-7H-pyrrolo[2,3-d]pyrimidin-4-amine 147b (5 mg, 0.01 mmol, 1.0 eq) and 4M HCl in MeOH (2 mL) at room temperature. The reaction was stirred at 40° C. for 30 min, then cooled and concentrated. The residue was dissolved in CH₃CN (1.0 mL) and was purified by Prep-HPLC with the following conditions (Column: Welch Ultimate XB-C18, 50*250 cm, 10 μm; Mobile Phase A: 0.1% HCl, Mobile Phase B: ACETONITRILE; Flow rate: 30 mL/min; Gradient: 5% B-2 min, 5% B-33% B-12 min; Wavelength: 254 nm) to afford (1R,2S,3R,5R)-3-(4-amino-2-chloro-7H-pyrrolo[2,3-d]pyrimidin-7-yl)-5-(1-phenethyl-1H-pyrazol-4-yl)cyclopentane-1,2-diol HCl salt 584 (2.6 mg, 57%) as a white solid. LCMS (Cortecs C18+, 50*3.0 mm, 2.7 μm, Mobile Phase A: Water/0.02% TFA, Mobile Phase B: Acetonitrile, 5%-95% B-5 min, 1.50 mL/min): T_{R}=2.65 min; ES m/z [M+H]⁺: 439, 441. ¹H NMR (400 MHZ, DMSO-d₆+D₂O) δ7.56 (s, 1H), 7.44 (s, 1H), 7.28-7.17 (m, 4H), 7.15-7.11 (m, 2H), 6.59 (d, J=3.5 Hz, 1H), 4.82-4.71 (m, 1H), 4.27 (t, J=7.4 Hz, 2H), 4.22-4.14 (m, 1H), 3.81 (t, J=5.7 Hz, 1H), 3.06 (t, J=7.4 Hz, 2H), 2.99-2.93 (m, 1H), 2.42-2.35 (m, 1H), 1.86-1.75 (m, 1H).

### Synthesis of (1R,2S,3R,5R)-3-[5-(4-benzyl-1,3-thiazol-2-yl)-2-chloropyrrolo[2,3-d]pyrimidin-7-yl]-5-(1-methyl-5,6-dihydro-2H-pyridin-3-yl)cyclopentane-1,2-diol (585)

### Tert-butyl 3-[(3aR,4R,6R,6aS)-6-{2-chloro-5-iodopyrrolo[2,3-d]pyrimidin-7-yl}-2,2-dimethyl-tetrahydro-3aH-cyclopenta[d][1,3]dioxol-4-yl]-5,6-dihydro-2H-pyridine-1-carboxylate (155b)

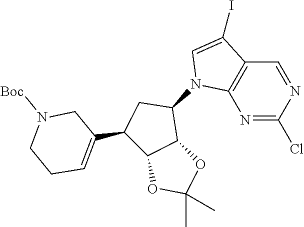 [SMILES: CC(C)(C)OC(=O)N1CCC=C([C@H]2C[C@@H](n3cc(I)c4cnc(Cl)nc43)[C@@H]3OC(C)(C)O[C@@H]32)C1]

Into a 40 mL vial were added 2-chloro-5-iodo-7H-pyrrolo[2,3-d]pyrimidine (0.20 g, 0.7 mmol, 1.0 eq), tert-butyl 3-[(3aR,4R,6S,6aS)-6-hydroxy-2,2-dimethyl-tetrahydro-3aH-cyclopenta[d][1,3]dioxol-4-yl]-5,6-dihydro-2H-pyridine-1-carboxylate 103b (0.30 g, 0.9 mmol, 1.3 eq) and PPh₃ (0.38 g, 1.4 mmol, 2.0 eq), followed by DBAD (0.33 g, 1.4 mmol, 2.0 eq) in toluene (6.0 mL) at 0° C. The resulting mixture was stirred for 2 h at 50° C., then concentrated. The residue was dissolved in DCM (2 mL) and was purified by silica gel column chromatography (1:1 Petroleum ether: AcOEt) to afford tert-butyl 3-[(3aR,4R,6R,6aS)-6-{2-chloro-5-iodopyrrolo[2,3-d]pyrimidin-7-yl}-2,2-dimethyl-tetrahydro-3aH-cyclopenta[d][1,3]dioxol-4-yl]-5,6-dihydro-2H-pyridine-1-carboxylate 155b (0.30 g, 69%) as a white solid. LCMS (conditions: XBridge C18, 50*3.0 mm, 3.5 μm, Mobile Phase A: Water/5 mM NH₄HCO₃, Mobile Phase B: Methanol, 10%-95% B-2 min, 1.50 mL/min): T_{R}=1.52 min; ES m/z [M+H]⁺: 601, 603.

### Tert-butyl 3-[(3aR,4R,6R,6aS)-6-[5-(4-benzyl-1,3-thiazol-2-yl)-2-chloropyrrolo[2,3-d]pyrimidin-7-yl]-2,2-dimethyl-tetrahydro-3aH-cyclopenta[d][1,3]dioxol-4-yl]-5,6-dihydro-2H-pyridine-1-carboxylate (156b)

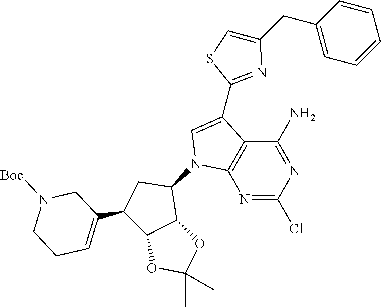 [SMILES: CC(C)(C)OC(=O)N1CCC=C([C@H]2C[C@@H](n3cc(-c4nc(Cc5ccccc5)cs4)c4c(N)nc(Cl)nc43)[C@@H]3OC(C)(C)O[C@@H]32)C1]

Into a 40 mL vial were added tert-butyl 3-[(3aR,4R,6R,6aS)-6-{2-chloro-5-iodopyrrolo[2,3-d]pyrimidin-7-yl}-2,2-dimethyl-tetrahydro-3aH-cyclopenta[d][1,3]dioxol-4-yl]-5,6-dihydro-2H-pyridine-1-carboxylate 155b (0.30 g, 0.5 mmol, 1.0 eq), (4-benzyl-1,3-thiazol-2-yl)(chloro) zinc (15 mmol, 3.0 eq) and Pd (PPh₃) 2Cl₂ (70.1 mg, 0.1 mmol, 0.2 eq) in toluene (3 mL) at room temperature. The resulting mixture was stirred for 8 h at 60° C. The resulting mixture was extracted with EtOAc (3×10.0 mL). The organic phases were dried over anhydrous Na₂SO₄ and concentrated. The residue was dissolved in DCM (2 mL) and was purified by silica gel column chromatography (3:2 Petroleum ether: AcOEt) to afford tert-butyl 3-[(3aR,4R,6R,6aS)-6-[5-(4-benzyl-1,3-thiazol-2-yl)-2-chloropyrrolo[2,3-d]pyrimidin-7-yl]-2,2-dimethyl-tetrahydro-3aH-cyclopenta[d][1,3]dioxol-4-yl]-5,6-dihydro-2H-pyridine-1-carboxylate 156b (0.25 g, 75%) as a white solid. LCMS (conditions: XBridge C18, 50*3.0 mm, 3.5 μm, Mobile Phase A: Water/5 mM NH₄HCO₃, Mobile Phase B: Methanol, 10%-95% B-2 min, 1.50 ml/min): T_{R}=1.75 min; ES m/z [M+H]⁺: 648, 650.

### (1R,2S,3R,5R)-3-[5-(4-Benzyl-1,3-thiazol-2-yl)-2-chloropyrrolo[2,3-d]pyrimidin-7-yl]-5-(1,2,5,6-tetrahydropyridin-3-yl)cyclopentane-1,2-diol (157b)

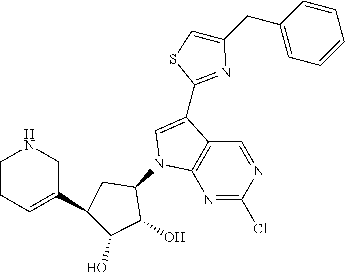 [SMILES: O[C@@H]1[C@H](O)[C@@H](C2=CCCNC2)C[C@H]1n1cc(-c2nc(Cc3ccccc3)cs2)c2cnc(Cl)nc21]

Into an 8 mL vial were added tert-butyl 3-[(3aR,4R,6R,6aS)-6-[5-(4-benzyl-1,3-thiazol-2-yl)-2-chloropyrrolo[2,3-d]pyrimidin-7-yl]-2,2-dimethyl-tetrahydro-3aH-cyclopenta[d][1,3]dioxol-4-yl]-5,6-dihydro-2H-pyridine-1-carboxylate 156b (0.25 g, 0.5 mmol, 1.0 eq) and 4M HCl in MeOH (3 mL) at room temperature. The reaction was stirred for 0.5 h at 50° C. The resulting mixture was concentrated to afford (1R,2S,3R,5R)-3-[5-(4-benzyl-1,3-thiazol-2-yl)-2-chloropyrrolo[2,3-d]pyrimidin-7-yl]-5-(1,2,5,6-tetrahydropyridin-3-yl)cyclopentane-1,2-diol 157b (200 mg, crude) as a yellow solid. LCMS (conditions: XBridge C18, 50*3.0 mm, 3.5 μm, Mobile Phase A: Water/5 mM NH₄HCO₃, Mobile Phase B: Methanol, 10%-95% B-2 min, 1.50 mL/min): T_{R}=1.80 min; ES m/z [M+H]⁺: 508, 510.

### (1R,2S,3R,5R)-3-[5-(4-Benzyl-1,3-thiazol-2-yl)-2-chloropyrrolo[2,3-d]pyrimidin-7-yl]-5-(1-methyl-5,6-dihydro-2H-pyridin-3-yl)cyclopentane-1,2-diol (585)

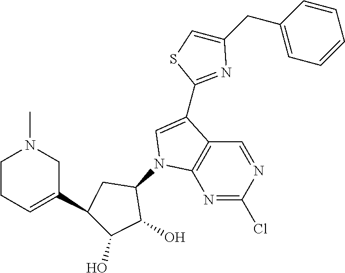 [SMILES: CN1CCC=C([C@H]2C[C@@H](n3cc(-c4nc(Cc5ccccc5)cs4)c4cnc(Cl)nc43)[C@H](O)[C@@H]2O)C1]

Into an 8 mL vial were added (1R,2S,3R,5R)-3-[5-(4-benzyl-1,3-thiazol-2-yl)-2-chloropyrrolo[2,3-d]pyrimidin-7-yl]-5-(1,2,5,6-tetrahydropyridin-3-yl)cyclopentane-1,2-diol 157b (80.0 mg, 0.15 mmol, 1.0 eq), 30% aqueous HCHO (31.5 mg, 0.3 mmol, 2.0 eq) and DIEA (40.7 mg, 0.3 mmol, 2.0 eq) in DCM (2 mL), followed by NaBH (OAc) 3 (66.7 mg, 0.3 mmol, 2.0 eq) at room temperature. The resulting mixture was stirred for 0.5 h, then concentrated. The residue was dissolved in MeOH (3 mL) and was purified by Prep-HPLC [column, Xbridge Prep C18 OBD column, 5 μm, 19*150 mm; mobile phase, Water (0.03% NH₄OH) and CH₃CN (20-40% over 10 min); Detector, UV 220&254 nm]to afford (1R,2S,3R,5R)-3-[5-(4-benzyl-1,3-thiazol-2-yl)-2-chloropyrrolo[2,3-d]pyrimidin-7-yl]-5-(1-methyl-5,6-dihydro-2H-pyridin-3-yl)cyclopentane-1,2-diol 585 (30.6 mg, 37%) as an off-white solid. LCMS (conditions: ZORBAX SB-Aq, 50*4.6 mm, 1.8 μm, Mobile Phase A: Water/0.02% TFA, Mobile Phase B: Acetonitrile, 5%-95% B-3 min, 1.50 mL/min): T_{R}=1.52 min; ES m/z [M+H]⁺: 522, 524. ¹H NMR (400 MHZ, DMSO-d₆) δ 9.32 (s, 1H), 8.63 (s, 1H), 7.40-7.30 (m, 4H), 7.28 (s, 1H), 7.25-7.20 (m, 1H), 5.62 (s, 1H), 5.05-4.78 (m, 3H), 4.24 (dd, J=8.3, 6.5 Hz, 1H), 4.15 (s, 2H), 3.87 (t, J=6.0 Hz, 1H), 2.98 (d, J=15.9 Hz, 1H), 2.79 (d, J=15.9 Hz, 1H), 2.33-2.47 (m, 3H), 2.27 (s, 3H), 2.16-2.06 (m, 3H), 1.99 (q, J=11.6 Hz, 1H).

### Synthesis of (1R,2S,3R,5R)-3-[5-(4-benzyl-1,3-thiazol-2-yl)-2-chloropyrrolo[2,3-d]pyrimidin-7-yl]-5-[1-(cyclopropylmethyl)-5,6-dihydro-2H-pyridin-3-yl]cyclopentane-1,2-diol (586)

### (1R,2S,3R,5R)-3-[5-(4-Benzyl-1,3-thiazol-2-yl)-2-chloropyrrolo[2,3-d]pyrimidin-7-yl]-5-[1-(cyclopropylmethyl)-5,6-dihydro-2H-pyridin-3-yl]cyclopentane-1,2-diol (586)

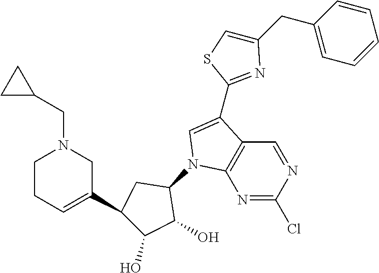 [SMILES: O[C@@H]1[C@H](O)[C@@H](C2=CCCN(CC3CC3)C2)C[C@H]1n1cc(-c2nc(Cc3ccccc3)cs2)c2cnc(Cl)nc21]

Into an 8 mL vial were added (1R,2S,3R,5R)-3-[5-(4-benzyl-1,3-thiazol-2-yl)-2-chloropyrrolo[2,3-d]pyrimidin-7-yl]-5-(1,2,5,6-tetrahydropyridin-3-yl)cyclopentane-1,2-diol 157b (80 mg, 0.15 mmol, 1.0 eq), cyclopropanecarbaldehyde (22 mg, 0.3 mmol, 2.0 eq) and DIEA (40 mg, 0.3 mmol, 2.0 eq) in DCM (2 mL), followed by NaBH (OAc) 3 (67 mg, 0.3 mmol, 2.0 eq) at room temperature. The resulting mixture was stirred for 0.5 h, then concentrated. The residue was dissolved in MeOH (3 mL). The residue was purified by Prep-HPLC [column, Xbridge Prep C18 OBD column, 5 μm, 19*150 mm; mobile phase, Water (0.03% NH₄OH) and CH₃CN (18-55% over 10 min); Detector, UV 220&254 nm]to afford (1R,2S,3R,5R)-3-[5-(4-benzyl-1,3-thiazol-2-yl)-2-chloropyrrolo[2,3-d]pyrimidin-7-yl]-5-[1-(cyclopropylmethyl)-5,6-dihydro-2H-pyridin-3-yl]cyclopentane-1,2-diol 586 (17.5 mg, 20%) as an off-white solid. LCMS (conditions: ZORBAX SB-Aq, 50*4.6 mm, 1.8 μm, Mobile Phase A: Water/0.02% TFA, Mobile Phase B: Acetonitrile, 5%-95% B-3 min, 1.50 mL/min): T_{R}=1.61 min; ES m/z [M+H]⁺: 562, 564. ¹H NMR (300 MHZ, DMSO-d₆) δ 9.32 (s, 1H), 8.60 (s, 1H), 7.46-7.14 (m, 6H), 5.66 (s, 1H), 5.08-4.80 (m, 3H), 4.24 (q, J=6.9 Hz, 1H), 4.14 (s, 2H), 3.89 (q, J=5.8 Hz, 1H), 3.09-3.05 (m, 1H), 3.04-2.95 (s, 1H), 2.63-2.51 (m, 1H), 2.46-2.41 (m, 1H), 2.38-2.24 (m, 2H), 2.21-2.07 (m, 3H), 2.06-1.94 (m, 1H), 0.98-0.84 (m, 1H), 0.63-0.43 (m, 2H), 0.27-0.08 (m, 2H).

### Synthesis of (1R,2S,3R,5R)-3-[5-(4-benzyl-1,3-thiazol-2-yl)-2-chloropyrrolo[2,3-d]pyrimidin-7-yl]-5-(1-cyclopropyl-5,6-dihydro-2H-pyridin-3-yl)cyclopentane-1,2-diol (587)

### 3-[(3aR,6R,6aS)-6-[5-(4-Benzyl-1,3-thiazol-2-yl)-2-chloropyrrolo[2,3-d]pyrimidin-7-yl]-2,2-dimethyl-tetrahydro-3aH-cyclopenta[d][1,3]dioxol-4-yl]-1,2,5,6-tetrahydropyridine (158b)

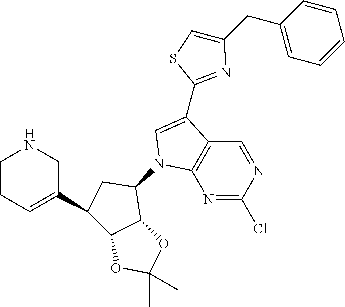 [SMILES: CC1(C)O[C@@H]2[C@H](O1)[C@@H](C1=CCCNC1)C[C@H]2n1cc(-c2nc(Cc3ccccc3)cs2)c2cnc(Cl)nc21]

Into an 8 mL vial were added (1R,2S,3R)-3-[5-(4-benzyl-1,3-thiazol-2-yl)-2-chloropyrrolo[2,3-d]pyrimidin-7-yl]-5-(1,2,5,6-tetrahydropyridin-3-yl)cyclopentane-1,2-diol 157b (0.1 g, 0.19 mmol, 1.0 eq), 2,2-dimethoxypropane (41.0 mg, 0.39 mmol, 2.0 eq) and p-TsOH (67.8 mg, 0.39 mmol, 2.0 eq) in acetone (3 mL). The resulting mixture was stirred for 1 h, then concentrated. The residue was dissolved in DCM (2 mL) and was purified by silica gel column chromatography (3:2 Petroleum ether: AcOEt) to afford 3-[(3aR,6R,6aS)-6-[5-(4-benzyl-1,3-thiazol-2-yl)-2-chloropyrrolo[2,3-d]pyrimidin-7-yl]-2,2-dimethyl-tetrahydro-3aH-cyclopenta[d][1,3]dioxol-4-yl]-1,2,5,6-tetrahydropyridine 158b (92 mg, 85%) as a white solid. LCMS (conditions XBridge C18, 50*3.0 mm, 3.5 μm, Mobile Phase A: Water/5 mM NH₄HCO₃, Mobile Phase B: Methanol, 10%-95% B-2 min, 1.50 mL/min): T_{R}=1.55 min; ES m/z [M+H]⁺: 548, 550.

### 3-[(3aR,6R,6aS)-6-[5-(4-Benzyl-1,3-thiazol-2-yl)-2-chloropyrrolo[2,3-d]pyrimidin-7-yl]-2,2-dimethyl-tetrahydro-3aH-cyclopenta[d][1,3]dioxol-4-yl]-1-cyclopropyl-5,6-dihydro-2H-pyridine (159b)

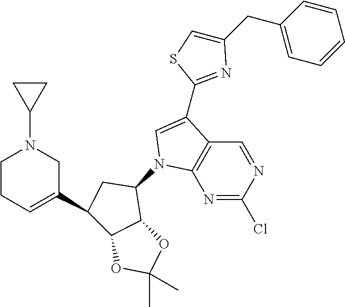 [SMILES: CC1(C)O[C@@H]2[C@H](O1)[C@@H](C1=CCCN(C3CC3)C1)C[C@H]2n1cc(-c2nc(Cc3ccccc3)cs2)c2cnc(Cl)nc21]

Into an 8 mL vial were added 3-[(3aR,6R,6aS)-6-[5-(4-benzyl-1,3-thiazol-2-yl)-2-chloropyrrolo[2,3-d]pyrimidin-7-yl]-2,2-dimethyl-tetrahydro-3aH-cyclopenta[d][1,3]dioxol-4-yl]-1,2,5,6-tetrahydropyridine 158b (98 mg, 0.18 mmol, 1.0 eq), cyclopropanol (31.2 mg, 0.54 mmol, 3.0 eq), Zn (CN) 2 (84.0 mg, 0.72 mmol, 4.0 eq) and Na₂CO₃ (75.8 mg, 0.72 mmol, 4.0 eq) in dioxane (2 mL) at room temperature. The reaction was stirred for 12 h at 100° C. The resulting mixture was filtered and the filtrate was concentrated. The residue was dissolved in DCM (2 mL) and was purified by silica gel column chromatography (1:1 Petroleum ether: AcOEt) to afford 3-[(3aR,6R,6aS)-6-[5-(4-benzyl-1,3-thiazol-2-yl)-2-chloropyrrolo[2,3-d]pyrimidin-7-yl]-2,2-dimethyl-tetrahydro-3aH-cyclopenta[d][1,3]dioxol-4-yl]-1-cyclopropyl-5,6-dihydro-2H-pyridine 159b (49 mg, 46%) as a white solid. LCMS (conditions: XBridge C18, 50*3.0 mm, 3.5 μm, Mobile Phase A: Water/5 mM NH₄HCO₃, Mobile Phase B: Methanol, 10%-95% B-2 min, 1.50 mL/min): T_{R}=1.82 min; ES m/z [M+H]⁺: 588, 590.

### (1R,2S,3R,5R)-3-[5-(4-Benzyl-1,3-thiazol-2-yl)-2-chloropyrrolo[2,3-d]pyrimidin-7-yl]-5-(1-cyclopropyl-5,6-dihydro-2H-pyridin-3-yl)cyclopentane-1,2-diol (587)

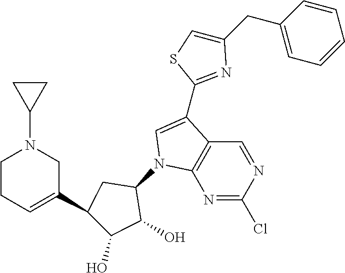 [SMILES: O[C@@H]1[C@H](O)[C@@H](C2=CCCN(C3CC3)C2)C[C@H]1n1cc(-c2nc(Cc3ccccc3)cs2)c2cnc(Cl)nc21]

Into an 8 mL vial were added 3-[(3aR,4R,6R,6aS)-6-[5-(4-benzyl-1,3-thiazol-2-yl)-2-chloropyrrolo[2,3-d]pyrimidin-7-yl]-2,2-dimethyl-tetrahydro-3aH-cyclopenta[d][1,3]dioxol-4-yl]-1-cyclopropyl-5,6-dihydro-2H-pyridine 159b (49 mg, 0.08 mmol, 1.0 eq) and 4M HCl in MeOH (2 mL) at room temperature. The reaction was stirred for 0.5 h, then concentrated. The residue was dissolved in MeOH (3 mL) and basified to pH>8 with NH₃·H₂O. The mixture was purified by Prep-HPLC [column, Xbridge Prep C18 OBD column, 5 μm, 19*150 mm; mobile phase, Water (0.03% NH₄OH) and CH₃CN (15-55% over 10 min); Detector, UV 220&254 nm]to afford (1R,2S,3R,5R)-3-[5-(4-benzyl-1,3-thiazol-2-yl)-2-chloropyrrolo[2,3-d]pyrimidin-7-yl]-5-(1-cyclopropyl-5,6-dihydro-2H-pyridin-3-yl)cyclopentane-1,2-diol 587 (9.6 mg, 21%) as a white solid. LCMS (conditions: ZORBAX SB-Aq, 50*4.6 mm, 1.8 μm, Mobile Phase A: Water/0.02% TFA, Mobile Phase B: Acetonitrile, 5%-95% B-3 min, 1.50 mL/min): T_{R}=1.16 min; ES m/z [M+H]⁺: 548, 550. ¹H NMR (300 MHZ, DMSO-d₆) δ 9.32 (s, 1H), 8.64 (s, 1H), 7.42-7.28 (m, 6H), 5.61 (s, 1H), 5.03-4.80 (m, 3H), 4.24 (q, J=6.8 Hz, 1H), 4.15 (s, 2H), 3.88 (q, J=5.7 Hz, 1H), 3.18 (d, J=15.9 Hz, 1H), 3.03 (d, J=16.2 Hz, 1H), 2.64 (t, J=5.7 Hz, 2H), 2.50-2.45 (m, 1H), 2.08-1.91 (m, 4H), 1.712 (s, 1H), 0.44-0.35 (m, 4H).

### Synthesis of (1R,2S,3R,5R)-3-[5-(4-benzyl-1,3-thiazol-2-yl)-2-chloropyrrolo[2,3-d]pyrimidin-7-yl]-5-[1-(2-phenylethyl)-5,6-dihydro-2H-pyridin-3-yl]cyclopentane-1,2-diol (588)

### (1R,2S,3R,5R)-3-[5-(4-Benzyl-1,3-thiazol-2-yl)-2-chloropyrrolo[2,3-d]pyrimidin-7-yl]-5-[1-(2-phenylethyl)-5,6-dihydro-2H-pyridin-3-yl]cyclopentane-1,2-diol (588)

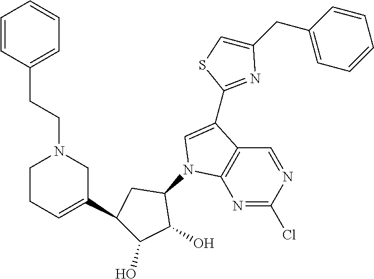 [SMILES: O[C@@H]1[C@H](O)[C@@H](C2=CCCN(CCc3ccccc3)C2)C[C@H]1n1cc(-c2nc(Cc3ccccc3)cs2)c2cnc(Cl)nc21]

Into an 8 mL vial were added (1R,2S,3R,5R)-3-[5-(4-benzyl-1,3-thiazol-2-yl)-2-chloropyrrolo[2,3-d]pyrimidin-7-yl]-5-(1,2,5,6-tetrahydropyridin-3-yl)cyclopentane-1,2-diol 157b (80.0 mg, 0.15 mmol, 1.0 eq), phenylacetaldehyde (56.7 mg, 0.45 mmol, 3.0 eq) and DIEA (40.7 mg, 0.3 mmol, 2.0 eq) in DCM (2 mL), followed by NaBH (OAc) 3 (66.7 mg, 0.3 mmol, 2.0 eq) at room temperature. The reaction was stirred for 1 h, then concentrated. The residue was purified by Prep-HPLC [column, Xbridge Prep C18 OBD column, 5 μm, 19*150 mm; mobile phase, Water (0.03% NH₄OH) and CH₃CN (20-60% over 10 min); Detector, UV 220&254 nm]to afford (1R,2S,3R,5R)-3-[5-(4-benzyl-1,3-thiazol-2-yl)-2-chloropyrrolo[2,3-d]pyrimidin-7-yl]-5-[1-(2-phenylethyl)-5,6-dihydro-2H-pyridin-3-yl]cyclopentane-1,2-diol 588 (34.8 mg, 36%) as a white solid. LCMS (conditions: ZORBAX SB-Aq, 50*4.6 mm, 1.8 μm, Mobile Phase A: Water/0.02% TFA, Mobile Phase B: Acetonitrile, 5%-95% B-3 min, 1.50 mL/min): T_{R}=1.69 min; ES m/z [M+H]⁺: 612, 614.

¹H NMR (400 MHZ, DMSO-d₆) δ 9.32 (s, 1H), 8.62 (s, 1H), 7.41-7.30 (m, 4H), 7.34-7.18 (m, 7H), 5.65 (s, 1H), 4.99-4.87 (m, 3H), 4.25 (t, J=7.5 Hz, 1H), 4.15 (s, 2H), 3.92-3.87 (m, 1H), 3.12 (d, J=15.9 Hz, 1H), 2.95 (d, J=15.9 Hz, 1H), 2.85-2.76 (m, 2H), 2.67-2.60 (m, 2H), 2.60-2.52 (m, 2H), 2.51-2.42 (m, 1H), 2.21-2.07 (m, 3H), 1.99 (q, J=11.6 Hz, 1H).

### Synthesis of (1R,2S,3R,5R)-3-[4-amino-5-(4-benzyl-1,3-thiazol-2-yl)pyrrolo[2,3-d]pyrimidin-7-yl]-5-(pyrrolidin-3-yl)cyclopentane-1,2-diol (589R) & (1R,2S,3R,5R)-3-[4-amino-5-(4-benzyl-1,3-thiazol-2-yl)pyrrolo[2,3-d]pyrimidin-7-yl]-5-[(3S)-pyrrolidin-3-yl]cyclopentane-1,2-diol (589S)

### Tert-butyl 3-[(3aR,4R,6aR)-2,2-dimethyl-6-oxo-tetrahydrocyclopenta[d][1,3]dioxol-4-yl]-2,5-dihydropyrrole-1-carboxylate (160b)

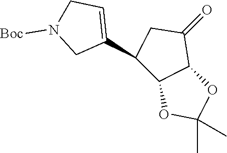 [SMILES: CC(C)(C)OC(=O)N1CC=C([C@H]2CC(=O)[C@@H]3OC(C)(C)O[C@H]23)C1]

Into a 40 mL vial were added (3aR,6aR)-2,2-dimethyl-3aH,6aH-cyclopenta[d][1,3]dioxol-4-one (1.5 g, 9.6 mmol, 1.0 eq), tert-butyl 3-(4,4,5,5-tetramethyl-1,3,2-dioxaborolan-2-yl)-2,5-dihydropyrrole-1-carboxylate (4.0 g, 13.6 mmol, 1.4 eq) and [RhCl (COD)]2 (0.14 g, 0.3 mmol, 0.03 eq), 2M KOH (630 μL, 11.2 mmol, 1.15 eq) in MeOH (15.0 mL). The final reaction mixture was irradiated with microwave radiation for 2 h at 40° C., then concentrated. The residue was dissolved in DCM (5 mL) and was purified by silica gel column chromatography (5:5 Petroleum ether: AcOEt) to afford tert-butyl 3-[(3aR,4R,6aR)-2,2-dimethyl-6-oxo-tetrahydrocyclopenta[d][1,3]dioxol-4-yl]-2,5-dihydropyrrole-1-carboxylate 160b (1.3 g, 88%) as a white solid. LCMS (conditions: XBridge C18, 50*3.0 mm, 3.5 μm, Mobile Phase A: Water/5 mM NH₄HCO₃, Mobile Phase B: Methanol, 10%-95% B-2 min, 1.50 mL/min): T_{R}=1.41 min; [M−H]: 322.

### Tert-butyl 3-[(3aR,4R,6aR)-2,2-dimethyl-6-oxo-tetrahydrocyclopenta[d][1,3]dioxol-4-yl]pyrrolidine-1-carboxylate (161b)

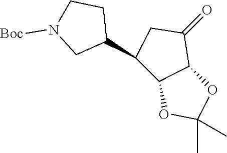 [SMILES: CC(C)(C)OC(=O)N1CCC([C@H]2CC(=O)[C@@H]3OC(C)(C)O[C@H]23)C1]

To a solution of tert-butyl 3-[(3aR,4R,6aR)-2,2-dimethyl-6-oxo-tetrahydrocyclopenta[d][1,3]dioxol-4-yl]-2,5-dihydropyrrole-1-carboxylate 160b (1.3 g, 4.0 mmol, 1.0 eq) in MeOH (13 mL) was added 10% Pd/C (0.3 g) in a pressure tank. The mixture was hydrogenated at room temperature under 30 psi of hydrogen pressure for 12 h, filtered through a Celite pad and concentrated to afford tert-butyl 3-[(3aR,4R,6aR)-2,2-dimethyl-6-oxo-tetrahydrocyclopenta[d][1,3]dioxol-4-yl]pyrrolidine-1-carboxylate 161b (1.2 g, 92%) as a white solid. LCMS (conditions: XBridge C18, 50*3.0 mm, 3.5 μm, Mobile Phase A: Water/5 mM NH₄HCO₃, Mobile Phase B: Methanol, 10%-95% B-2 min, 1.50 mL/min): T_{R}=1.41 min; [M−H]: 324.

### Tert-butyl 3-[(3aR,4R,6S,6aS)-6-hydroxy-2,2-dimethyl-tetrahydro-3aH-cyclopenta[d][1,3]dioxol-4-yl]pyrrolidine-1-carboxylate (162b)

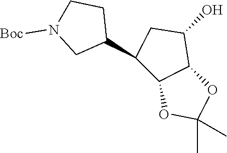 [SMILES: CC(C)(C)OC(=O)N1CCC([C@H]2C[C@H](O)[C@@H]3OC(C)(C)O[C@@H]32)C1]

Into a 40 mL vial was added tert-butyl 3-[(3aR,4R,6aR)-2,2-dimethyl-6-oxo-tetrahydrocyclopenta[d][1,3]dioxol-4-yl]pyrrolidine-1-carboxylate 161b (1.5 g, 4.6 mmol, 1.0 eq) in MeOH (15.0 mL), followed by NaBH₄ (0.17 g, 4.6 mmol, 1.0 eq) at 0° C. The reaction was stirred for 0.5 h at room temperature, then concentrated. The residue was dissolved in DCM (4.0 mL) and was purified by silica gel column chromatography (5:5 Petroleum ether: AcOEt) to afford tert-butyl 3-[(3aR,4R,6S,6aS)-6-hydroxy-2,2-dimethyl-tetrahydro-3aH-cyclopenta[d][1,3]dioxol-4-yl]pyrrolidine-1-carboxylate 162b (0.86 g, 57%) as a white solid. LCMS (conditions: XBridge C18, 50*3.0 mm, 3.5 μm, Mobile Phase A: Water/5 mM NH₄HCO₃, Mobile Phase B: Methanol, 10%-95% B-2 min, 1.50 mL/min): T_{R}=1.41 min; [M−H]: 326.

### Tert-butyl 3-[(3aR,4R,6R,6aS)-6-{4-chloro-5-iodopyrrolo[2,3-d]pyrimidin-7-yl}-2,2-dimethyl-tetrahydro-3aH-cyclopenta[d][1,3]dioxol-4-yl]piperidine-1-carboxylate (163b)

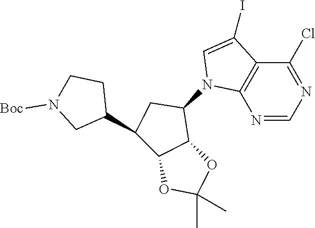 [SMILES: CC(C)(C)OC(=O)N1CCC([C@H]2C[C@@H](n3cc(I)c4c(Cl)ncnc43)[C@@H]3OC(C)(C)O[C@@H]32)C1]

Into a 40 mL vial were added 4-chloro-5-iodo-7H-pyrrolo[2,3-d]pyrimidine (0.54 g, 1.9 mmol, 1.0 eq), tert-butyl 3-[(3aR,4R,6S,6aS)-6-hydroxy-2,2-dimethyl-tetrahydro-3aH-cyclopenta[d][1,3]dioxol-4-yl]piperidine-1-carboxylate 162 (0.86 g, 2.5 mmol, 1.3 eq) and DBAD (0.9 g, 3.9 mmol, 2.0 eq), followed by PPh₃ (1.0 g, 3.9 mmol, 2.0 eq) in toluene (10 mL) at 0° C. The reaction was stirred for 6 h at 60° C., then concentrated. The residue was dissolved in DCM (3 mL) and was purified by silica gel column chromatography (6:4 Petroleum ether: AcOEt) to afford to afford tert-butyl 3-[(3aR,4R,6R,6aS)-6-{4-chloro-5-iodopyrrolo[2,3-d]pyrimidin-7-yl}-2,2-dimethyl-tetrahydro-3aH-cyclopenta[d][1,3]dioxol-4-yl]piperidine-1-carboxylate 163b (0.70 g, 60%) as a white solid. LCMS (conditions Xbridge Shield C18, 50*3.0 mm, 3.5 μm, Mobile Phase A: Water/0.05% Ammonia, Mobile Phase B: Acetonitrile, 5%-100% B-2 min, 1.50 mL/min): T_{R}=1.62 min; ES m/z [M+H]⁺: 589, 591.

### Tert-butyl 3-[(3aR,4R,6R,6aS)-6-{4-amino-5-iodopyrrolo[2,3-d]pyrimidin-7-yl}-2,2-dimethyl-tetrahydro-3aH-cyclopenta[d][1,3]dioxol-4-yl]pyrrolidine-1-carboxylate (164b)

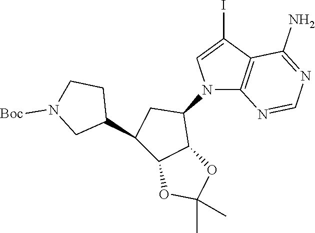 [SMILES: CC(C)(C)OC(=O)N1CCC([C@H]2C[C@@H](n3cc(I)c4c(N)ncnc43)[C@@H]3OC(C)(C)O[C@@H]32)C1]

Into a 40 mL vial were added tert-butyl 3-[(3aR,4R,6R,6aS)-6-{4-chloro-5-iodopyrrolo[2,3-d]pyrimidin-7-yl}-2,2-dimethyl-tetrahydro-3aH-cyclopenta[d][1,3]dioxol-4-yl]pyrrolidine-1-carboxylate 163b (0.70 g, 1.2 mmol, 1.0 eq) and 7M NH₃ in MeOH (5 mL) at room temperature. The resulting mixture was stirred for 2 h at 80° C., then concentrated. The residue was dissolved in DCM (3 mL) and was purified by silica gel column chromatography (1:1 Petroleum ether: AcOEt) to afford tert-butyl 3-[(3aR,4R,6R,6aS)-6-{4-amino-5-iodopyrrolo[2,3-d]pyrimidin-7-yl}-2,2-dimethyl-tetrahydro-3aH-cyclopenta[d][1,3]dioxol-4-yl]pyrrolidine-1-carboxylate 164b (0.50 g, 74%) as a white solid. LCMS (conditions XBridge C18, 50*3.0 mm, 3.5 μm, Mobile Phase A: Water/5 mM NH₄HCO₃, Mobile Phase B: Methanol, 10%-95% B-2 min, 1.50 ml/min): T_{R}=1.19 min; ES m/z [M+H]⁺: 570.

### Tert-butyl 3-[(3aR,4R,6R,6aS)-6-[4-amino-5-(4-benzyl-1,3-thiazol-2-yl)pyrrolo[2,3-d]pyrimidin-7-yl]-2,2-dimethyl-tetrahydro-3aH-cyclopenta[d][1,3]dioxol-4-yl]pyrrolidine-1-carboxylate (165b)

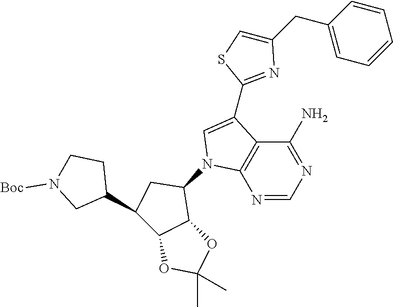 [SMILES: CC(C)(C)OC(=O)N1CCC([C@H]2C[C@@H](n3cc(-c4nc(Cc5ccccc5)cs4)c4c(N)ncnc43)[C@@H]3OC(C)(C)O[C@@H]32)C1]

Into a 40 mL vial were added tert-butyl 3-[(3aR,4R,6R,6aS)-6-{4-amino-5-iodopyrrolo[2,3-d]pyrimidin-7-yl}-2,2-dimethyl-tetrahydro-3aH-cyclopenta[d][1,3]dioxol-4-yl]pyrrolidine-1-carboxylate 164b (0.50 g, 0.9 mmol, 1.0 eq), (4-benzyl-1,3-thiazol-2-yl)(chloro) zinc (2.7 mmol, 3.0 eq) and Pd (PPh₃) 2Cl₂ (0.12 q, 0.2 mmol, 0.2 eq) in toluene (5 mL). The resulting mixture was stirred for 6 h at 60° C. The resulting mixture was extracted with EtOAc (3×10 mL). The combined organic phases were dried over anhydrous Na₂SO₄ and concentrated. The residue was dissolved in DCM (2 mL) and was purified by silica gel column chromatography (3:2 Petroleum ether: AcOEt) to afford tert-butyl 3-[(3aR,4R,6R,6aS)-6-[4-amino-5-(4-benzyl-1,3-thiazol-2-yl)pyrrolo[2,3-d]pyrimidin-7-yl]-2,2-dimethyl-tetrahydro-3aH-cyclopenta[d][1,3]dioxol-4-yl]pyrrolidine-1-carboxylate 165b (0.15 g, 27%) as a white solid. LCMS (conditions XBridge C18, 50*3.0 mm, 3.5 μm, Mobile Phase A: Water/5 mM NH₄HCO₃, Mobile Phase B: Methanol, 10%-95% B-2 min, 1.50 mL/min): T_{R}=1.60 min; ES m/z [M+H]⁺: 617.

### (1R,2S,3R,5R)-3-[4-Amino-5-(4-benzyl-1,3-thiazol-2-yl)pyrrolo[2,3-d]pyrimidin-7-yl]-5-(pyrrolidin-3-yl)cyclopentane-1,2-diol (589R) & (1R,2S,3R,5R)-3-[4-amino-5-(4-benzyl-1,3-thiazol-2-yl)pyrrolo[2,3-d]pyrimidin-7-yl]-5-[(3S)-pyrrolidin-3-yl]cyclopentane-1,2-diol (5895)

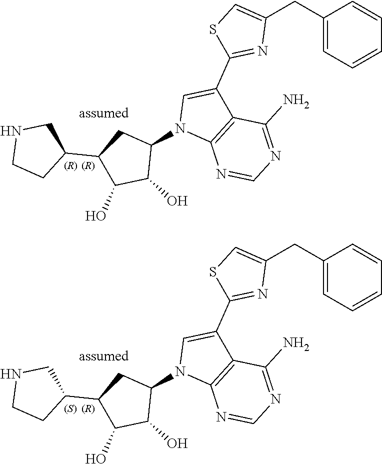

Into an 8 mL vial were added tert-butyl 3-[(3aR,4R,6R,6aS)-6-[4-amino-5-(4-benzyl-1,3-thiazol-2-yl)pyrrolo[2,3-d]pyrimidin-7-yl]-2,2-dimethyl-tetrahydro-3aH-cyclopenta[d][1,3]dioxol-4-yl]pyrrolidine-1-carboxylate 165b (0.15 g, 0.5 mmol, 1.0 eq) and 4M HCl in MeOH (1.5 mL). The resulting mixture was stirred for 0.5 h, then concentrated. The crude product (150 mg) was purified by Prep-HPLC with the following conditions (Column: XBridge C18 19*150 mm, 5 μm; Mobile Phase A: 0.05% NH₃·H₂O+10 mM NH₄HCO₃; Mobile Phase B: ACETONITRILE; Flow rate: 20 mL/min; Gradient: 25% B to 65% B over 10 min); Detector, UV 220&254 nm) to afford two diastereomers:

(1R,2S,3R,5R)-3-[4-Amino-5-(4-benzyl-1,3-thiazol-2-yl)pyrrolo[2,3-d]pyrimidin-7-yl]-5-[(3R)-pyrrolidin-3-yl]cyclopentane-1,2-diol 589R (5.0 mg, 4.3%, structure assumed) as a white solid. LCMS (conditions: ZORBAX SB-Aq, 50*4.6 mm, 1.8 μm, Mobile Phase A: Water/0.02% TFA, Mobile Phase B: Acetonitrile, 5%-95% B-3 min, 1.50 mL/min): T_{R}=1.19 min; ES m/z [M+H]⁺: 477.1H NMR (300 MHZ, DMSO-d₆+D₂O) δ 8.10 (s, 1H), 8.06 (s, 1H), 7.35-7.28 (m, 4H), 7.24-7.18 (m, 2H), 4.90-4.79 (m, 1H), 4.31-4.19 (m, 1H), 4.06 (s, 2H), 3.84-3.73 (m, 1H), 3.53-3.51 (m, 1H), 3.23-3.17 (m, 2H), 2.92-2.79 (m, 1H), 2.21-2.09 (m, 3H), 1.87-1.78 (m, 1H), 1.61-1.55 (m, 2H). (1R,2S,3R,5R)-3-[4-Amino-5-(4-benzyl-1,3-thiazol-2-yl)pyrrolo[2,3-d]pyrimidin-7-yl]-5-[(3S)-pyrrolidin-3-yl]cyclopentane-1,2-diol 5895 (9.3 mg, 8%, structure assumed) as a white solid. LCMS (conditions: ZORBAX SB-Aq, 50*4.6 mm, 1.8 μm Mobile Phase A: Water/0.02% TFA, Mobile Phase B: Acetonitrile, 5%-40%-95% B-7 min (+), 1.50 mL/min): T_{R}=3.35 min; ES m/z [M+H]⁺: 477.1H NMR (300 MHZ, DMSO-d₆+D₂O) δ 8.09 (s, 1H), 8.06 (s, 1H), 7.43-7.12 (m, 6H), 4.93-4.80 (m, 1H), 4.32-4.24 (m, 1H), 4.05 (s, 2H), 3.82-3.73 (m, 1H), 3.51-3.32 (m, 1H), 3.30-2.98 (m, 2H), 2.91-2.78 (m, 1H), 2.21-2.02 (m, 2H), 1.90-1.71 (m, 2H), 1.65-1.20 (m, 2H).

### Synthesis of 5-[(1R,2R,3S,4R)-4-{4-amino-2-chloropyrrolo[2,3-d]pyrimidin-7-yl}-2,3-dihydroxycyclopentyl]-1H-pyridin-2-one (590)

### (3aR,6R,6aR)-6-(6-Methoxypyridin-3-yl)-2,2-dimethyl-tetrahydrocyclopenta[d][1,3]dioxol-4-one (168b)

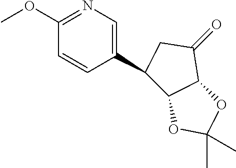

Into a 40 mL vial were added 6-methoxypyridin-3-ylboronic acid (0.59 g, 3.89 mmol, 1.5 eq), (3aR,6aR)-2,2-dimethyl-3aH,6aH-cyclopenta[d][1,3]dioxol-4-one (0.4 g, 2.59 mmol, 1.0 eq), acetylacetonato bis(ethylene) rhodium (I)(66.9 mg, 0.26 mmol, 0.1 eq) and R-BINAP (0.2 g, 0.26 mmol, 0.1 eq) in 1,4-dioxane (5 mL) and H₂O (0.5 mL). The reaction was irradiated with microwave radiation for 3 h at 100° C. The reaction was cooled, quenched with water (10 mL) and then extracted with EtOAc (3×15 mL). The organic layer was dried over anhydrous Na₂SO₄, filtered and the filtrate was concentrated. The residue was dissolved in DCM (4.0 mL) and was purified by silica gel column chromatography (2:1 Petroleum ether: AcOEt) to afford (3aR,6R,6aR)-6-(6-methoxypyridin-3-yl)-2,2-dimethyl-tetrahydrocyclopenta[d][1,3]dioxol-4-one 168b (0.49 g, 71%) as a white solid. LCMS (conditions XBridge C18, 50*3.0 mm, 3.5 μm, Mobile Phase A: Water/5 mM NH₄HCO₃, Mobile Phase B: Acetonitrile, 5%-100% B-2 min, 1.50 mL/min,): T_{R}=1.05 min; ES m/z [M+H]⁺: 264.

### (3aS,45,6R,6aR)-6-(6-Methoxypyridin-3-yl)-2,2-dimethyl-tetrahydro-3aH-cyclopenta[d][1,3]dioxol-4-ol (169b)

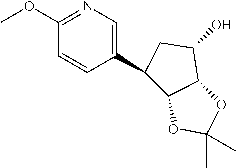

Into a 40 mL vial were added (3aR,6R,6aR)-6-(6-methoxypyridin-3-yl)-2,2-dimethyl-tetrahydrocyclopenta[d][1,3]dioxol-4-one 168b (0.49 g, 1.8 mmol, 1.0 eq) and NaBH₄ (0.1 g, 2.78 mmol, 1.5 eq) in MeOH (5 mL) at 0° C. The reaction was stirred for 0.5 h, then was quenched with water (10 mL) and extracted with EtOAc (3×15 mL). The organic layer was dried over anhydrous Na₂SO₄, filtered and the filtrate was concentrated. The residue was dissolved in DCM (3 mL) and was purified by silica gel column chromatography (2:1 Petroleum ether: AcOEt) to afford (3aS,4S,6R,6aR)-6-(6-methoxypyridin-3-yl)-2,2-dimethyl-tetrahydro-3aH-cyclopenta[d][1,3]dioxol-4-ol 169b (0.34 g, 68%) as a white solid. LCMS (conditions XBridge C18, 50*3.0 mm, 3.5 μm, Mobile Phase A: Water/5 mM NH₄HCO₃, Mobile Phase B: Methanol, 5%-100% B-2 min, 1.50 mL/min,): T_{R}=1.29 min; ES m/z [M+H]⁺: 266.

### 5-[(3aR,4R,6R,6aS)-6-{2,4-Dichloropyrrolo[2,3-d]pyrimidin-7-yl}-2,2-dimethyl-tetrahydro-3aH-cyclopenta[d][1,3]dioxol-4-yl]-2-methoxypyridine (170b)

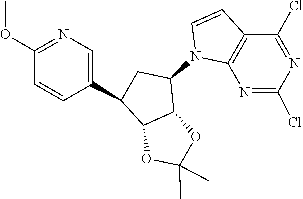 [SMILES: COc1ccc([C@H]2C[C@@H](n3ccc4c(Cl)nc(Cl)nc43)[C@@H]3OC(C)(C)O[C@@H]32)cn1]

Into an 8 mL vial were added (3aS,4S,6R,6aR)-6-(6-methoxypyridin-3-yl)-2,2-dimethyl-tetrahydro-3aH-cyclopenta[d][1,3]dioxol-4-ol 169b (0.34 g, 1.27 mmol, 1.3 eq), 2,4-dichloro-7H-pyrrolo[2,3-d]pyrimidine (0.18 g, 0.97 mmol, 1.0 eq) and PPh₃ (0.51 g, 1.95 mmol, 2.0 eq) in THF (3 mL), followed by DIAD (0.39 g, 1.95 mmol, 2.0 eq) at 0° C. The reaction was stirred for 12 h, then concentrated. The residue was dissolved in DCM (2 mL) and was purified by silica gel column chromatography (1:1 Petroleum ether: AcOEt) to afford 5-[(3aR,4R,6R,6aS)-6-{2,4-dichloropyrrolo[2,3-d]pyrimidin-7-yl}-2,2-dimethyl-tetrahydro-3aH-cyclopenta[d][1,3]dioxol-4-yl]-2-methoxypyridine 170 (0.21 g, 49%) as a white solid. LCMS (conditions XBridge C18, 50*3.0 mm, 3.5 μm, Mobile Phase A: Water/5 mM NH₄HCO₃, Mobile Phase B: Methanol, 5%-100% B-2 min, 1.50 mL/min,): T_{R}=1.61 min; ES m/z [M+H]⁺: 435, 437.

### 5-[(3aR,4R,6R,6aS)-6-{2,4-Dichloropyrrolo[2,3-d]pyrimidin-7-yl}-2,2-dimethyl-tetrahydro-3aH-cyclopenta[d][1,3]dioxol-4-yl]-1H-pyridin-2-one (171b)

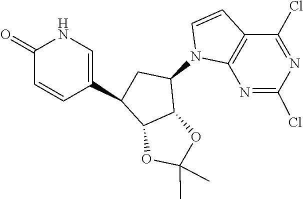 [SMILES: CC1(C)O[C@@H]2[C@H](O1)[C@@H](c1ccc(=O)[nH]c1)C[C@H]2n1ccc2c(Cl)nc(Cl)nc21]

Into an 8 mL vial were added 5-[(3aR,4R,6R,6aS)-6-{2,4-dichloropyrrolo[2,3-d]pyrimidin-7-yl}-2,2-dimethyl-tetrahydro-3aH-cyclopenta[d][1,3]dioxol-4-yl]-2-methoxypyridine 170b (0.21 g, 0.48 mmol, 1.0 eq), SiMe₃Cl (0.1 g, 0.96 mmol, 2.0 eq) and Nal (0.22 g, 1.45 mmol, 3.0 eq) in Acetonitrile (3 mL). The final reaction mixture was irradiated with microwave radiation for 10 min at 100° C., then concentrated. The residue was dissolved in DCM (2 mL) and was purified by silica gel column chromatography (1:1 Petroleum ether: AcOEt) to afford 5-[(3aR,4R,6R,6aS)-6-{2,4-dichloropyrrolo[2,3-d]pyrimidin-7-yl}-2,2-dimethyl-tetrahydro-3aH-cyclopenta[d][1,3]dioxol-4-yl]-1H-pyridin-2-one 171b (0.14 g, 70%) as a white solid. LCMS (conditions XBridge C18, 50*3.0 mm, 3.5 μm, Mobile Phase A: Water/5 mM NH₄HCO₃, Mobile Phase B: Methanol, 5%-100% B-2 min, 1.50 mL/min,): T_{R}=1.45 min; ES m/z [M+H]⁺: 421, 423.

### 5-[(3aR,4R,6R,6aS)-6-{4-Amino-2-chloropyrrolo[2,3-d]pyrimidin-7-yl}-2,2-dimethyl-tetrahydro-3aH-cyclopenta[d][1,3]dioxol-4-yl]-1H-pyridin-2-one (172b)

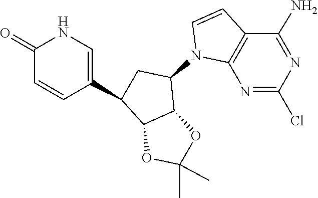 [SMILES: CC1(C)O[C@@H]2[C@H](O1)[C@@H](c1ccc(=O)[nH]c1)C[C@H]2n1ccc2c(N)nc(Cl)nc21]

Into an 8 mL vial were added 5-[(3aR,4R,6R,6aS)-6-{2,4-dichloropyrrolo[2,3-d]pyrimidin-7-yl}-2,2-dimethyl-tetrahydro-3aH-cyclopenta[d][1,3]dioxol-4-yl]-1H-pyridin-2-one 171b (0.14 g, 0.34 mmol, 1.0 eq) and 7M NH₃ in MeOH (2 mL). The reaction was stirred for 8 h at 80° C. The resulting mixture was concentrated to afford 5-[(3aR,4R,6R,6aS)-6-{4-amino-2-chloropyrrolo[2,3-d]pyrimidin-7-yl}-2,2-dimethyl-tetrahydro-3aH-cyclopenta[d][1,3]dioxol-4-yl]-1H-pyridin-2-one 172b (0.12 g, crude) as a yellow solid. LCMS (conditions XBridge C18, 50*3.0 mm, 3.5 μm, Mobile Phase A: Water/5 mM NH₄HCO₃, Mobile Phase B: Acetonitrile, 5%-100% B-2 min, 1.50 mL/min,): T_{R}=1.07 min; ES m/z [M+H]⁺: 402, 404.

### 5-[(1R,2R,3S,4R)-4-{4-Amino-2-chloropyrrolo[2,3-d]pyrimidin-7-yl}-2,3-dihydroxycyclopentyl]-1H-pyridin-2-one (590)

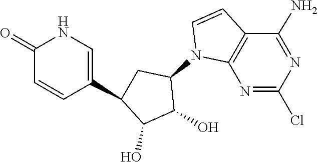 [SMILES: Nc1nc(Cl)nc2c1ccn2[C@@H]1C[C@H](c2ccc(=O)[nH]c2)[C@@H](O)[C@H]1O]

Into an 8 mL vial were added 5-[(3aR,4R,6R,6aS)-6-{4-amino-2-chloropyrrolo[2,3-d]pyrimidin-7-yl}-2,2-dimethyl-tetrahydro-3aH-cyclopenta[d][1,3]dioxol-4-yl]-1H-pyridin-2-one 172b (0.12 g, 0.30 mmol, 1.0 eq) and 4M HCl in MeOH (2 mL). The reaction was stirred for 0.5 h, then concentrated. The residue was dissolved in MeOH (3 mL) and basified to pH>8 with NH₃·H₂O, then purified by Prep-HPLC [column, Xbridge Prep C18 OBD column, 5 μm, 19*150 mm; mobile phase, Water (0.03% NH₄OH) and CH₃CN (20-60% over 10 min); Detector, UV 220&254 nm]to afford 5-[(1R,2R,3S,4R)-4-{4-amino-2-chloropyrrolo[2,3-d]pyrimidin-7-yl}-2,3-dihydroxycyclopentyl]-1H-pyridin-2-one 590 (31.1 mg, 29%) as an off-white solid. LCMS (conditions: ZORBAX SB-Aq, 50*4.6 mm, 1.8 μm, 5%-95% Acetonitrile/0.02% aqueous TFA-3 min, 1.50 mL/min): T_{R}=0.69 min; ES m/z [M+H]⁺: 362, 364. ¹H NMR (300 MHZ, DMSO-d₆) δ 11.42 (br, s, 1H), 7.61 (dd, J=9.5, 2.7 Hz, 1H), 7.46 (s, 2H), 7.38 (d, J=3.6 Hz, 1H), 7.24 (d, J=2.6 Hz, 1H), 6.59 (d, J=3.5 Hz, 1H), 6.35 (d, J=9.4 Hz, 1H), 4.95 (d, J=5.7 Hz, 1H), 4.88 (d, J=6.4 Hz, 1H), 4.84-4.70 (m, 1H), 4.13 (q, J=6.1 Hz, 1H), 3.94 (q, J=6.8 Hz, 1H), 2.93-2.79 (m, 1H), 2.33-2.18 (m, 1H), 1.83 (q, J=11.8 Hz, 1H).

### Synthesis of (1R,2S,3R,5R)-3-{4-amino-2-chloropyrrolo[2,3-d]pyrimidin-7-yl}-5-(6-methoxypyridin-3-yl)cyclopentane-1,2-diol (591)

### 7-[(3aS,4R,6R,6aR)-6-(6-Methoxypyridin-3-yl)-2,2-dimethyl-tetrahydro-3aH-cyclopenta[d][1,3]dioxol-4-yl]-2-chloropyrrolo [2,3-d]pyrimidin-4-amine (173b)

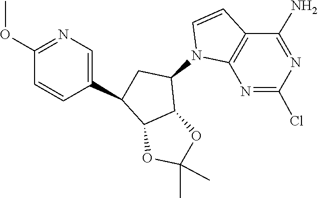 [SMILES: COc1ccc([C@H]2C[C@@H](n3ccc4c(N)nc(Cl)nc43)[C@@H]3OC(C)(C)O[C@@H]32)cn1]

Into an 8 mL vial were added 5-[(3aR,4R,6R,6aS)-6-{2,4-dichloropyrrolo[2,3-d]pyrimidin-7-yl}-2,2-dimethyl-tetrahydro-3aH-cyclopenta[d][1,3]dioxol-4-yl]-2-methoxypyridine 170b (60 mg, 0.14 mmol, 1.0 eq) and 7M NH₃ in MeOH (1.0 mL). The reaction was stirred for 8 h at 80° C., then concentrated. The residue was dissolved in DCM (1.0 mL) and was purified by silica gel column chromatography (1:1 Petroleum ether: AcOEt) to afford 7-[(3aS,4R,6R,6aR)-6-(6-methoxypyridin-3-yl)-2,2-dimethyl-tetrahydro-3aH-cyclopenta[d][1,3]dioxol-4-yl]-2-chloropyrrolo[2,3-d]pyrimidin-4-amine 173b (43 mg, 75%) as a white solid. LCMS (conditions XBridge C18, 50*3.0 mm, 3.5 μm, 5%-100% Acetonitrile/5 mM aqueous NH₄HCO₃-2 min, 1.50 mL/min,): T_{R}=1.05 min; ES m/z [M+H]⁺: 264. LCMS (conditions XBridge C18, 50*3.0 mm, 3.5 μm, Mobile Phase A: Water/5 mM NH₄HCO₃, Mobile Phase B: Acetonitrile, 5%-100% B-2 min, 1.50 mL/min,): T_{R}=1.26 min; ES m/z [M+H]⁺: 416, 418.

### (1R,2S,3R,5R)-3-{4-amino-2-chloropyrrolo[2,3-d]pyrimidin-7-yl}-5-(6-methoxypyridin-3-yl)cyclopentane-1,2-diol (591)

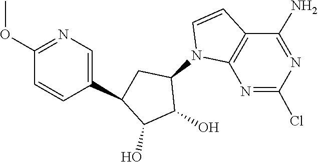 [SMILES: COc1ccc([C@H]2C[C@@H](n3ccc4c(N)nc(Cl)nc43)[C@H](O)[C@@H]2O)cn1]

Into an 8 mL vial were added 7-[(3aS,4R,6R,6aR)-6-(6-methoxypyridin-3-yl)-2,2-dimethyl-tetrahydro-3aH-cyclopenta[d][1,3]dioxol-4-yl]-2-chloropyrrolo[2,3-d]pyrimidin-4-amine 173b (43 mg, 0.10 mmol, 1.0 eq) and 4M HCl in MeOH (2 mL), and the reaction stirred for 0.5 hrs, before being concentrated. The residue was dissolved in MeOH (3 mL). The mixture was basified to pH >8 with NH₃·H₂O and was purified by Prep-HPLC [column, Xbridge Prep C18 OBD column, 5 μm, 19*150 mm; mobile phase, Water (0.03% NH₄OH) and CH₃CN (15-55% over 10 min); Detector, UV 220&254 nm]to afford (1R,2S,3R,5R)-3-{4-amino-2-chloropyrrolo[2,3-d]pyrimidin-7-yl}-5-(6-methoxypyridin-3-yl)cyclopentane-1,2-diol 591 (22.7 mg, 59%) as an off-white solid. LCMS (conditions: ZORBAX SB-Aq, 50*4.6 mm, 1.8 μm, 5%-95% Acetonitrile/0.02% aqueous TFA-3 min, 1.50 mL/min): T_{R}=0.95 min; ES m/z [M+H]⁺: 376, 378. ¹H NMR (400 MHZ, DMSO-d₆) δ 8.13 (d, J=2.4 Hz, 1H), 7.82 (dd, J=8.6, 2.6 Hz, 1H), 7.46 (s, 2H), 7.40 (d, J=3.5 Hz, 1H), 6.82 (d, J=8.6 Hz, 1H), 6.60 (d, J=3.5 Hz, 1H), 5.11-4.98 (m, 2H), 4.88-4.77 (m, 1H), 4.21 (t, J=6.4 Hz, 1H), 4.02 (t, J=6.8 Hz, 1H), 3.84 (s, 3H), 3.12-3.01 (m, 1H), 2.41-2.33 (m, 1H), 1.94 (q, J=11.8 Hz, 1H).

### Synthesis of (15,2R,3R,5R)-3-[3-(2-Aminoethyl)phenyl]-5-{4-aminopyrrolo[2,3-d]pyrimidin-7-yl}cyclopentane-1,2-diol (592)

### Tert-butyl N-{2-[3-(4,4,5,5-tetramethyl-1,3,2-dioxaborolan-2-yl)phenyl]ethyl}carbamate (174b)

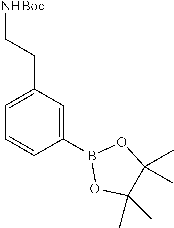 [SMILES: CC(C)(C)OC(=O)NCCc1cccc(B2OC(C)(C)C(C)(C)O2)c1]

Into a 40 mL vial were added tert-butyl N-[2-(3-bromophenyl)ethyl]carbamate (1 g, 3.3 mmol, 1.0 eq), 4,4,5,5-tetramethyl-2-(tetramethyl-1,3,2-dioxaborolan-2-yl)-1,3,2-dioxaborolane (1.27 g, 5.0 mmol, 1.5 eq), KOAc (0.65 g, 6.6 mmol, 2.0 eq) and Pd (dppf) Cl₂ (0.24 g, 0.33 mmol, 0.1 eq) in dioxane (10.0 mL). The reaction was stirred for 2 h at 100° C., then concentrated. The residue was dissolved in DCM (4.0 mL) and was purified by silica gel column chromatography (3:1 Petroleum ether: AcOEt) to afford tert-butyl N-{2-[3-(4,4,5,5-tetramethyl-1,3,2-dioxaborolan-2-yl)phenyl]ethyl}carbamate 174b (0.6 g, 52%) as a white solid. LCMS (conditions XBridge C18, 50*3.0 mm, 3.5 μm, 5%-100% Acetonitrile/5 mM aqueous NH₄HCO₃-2.5 min, 1.50 mL/min,) T_{R}=1.89 min; ES m/z [M+H]⁺: 348.

### Tert-butyl N-(2-{3-[(3aR,4R,6aR)-2,2-dimethyl-6-oxo-tetrahydrocyclopenta[d][1,3]dioxol-4-yl]phenyl}ethyl)carbamate (175b)

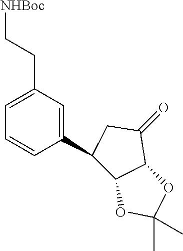 [SMILES: CC(C)(C)OC(=O)NCCc1cccc([C@H]2CC(=O)[C@@H]3OC(C)(C)O[C@H]23)c1]

Into a 40 mL vial were added (3aR,6aR)-2,2-dimethyl-3aH,6aH-cyclopenta[d][1,3]dioxol-4-one (0.18 g, 1.15 mmol, 1.0 eq), tert-butyl N-{2-[3-(4,4,5,5-tetramethyl-1,3,2-dioxaborolan-2-yl)phenyl]ethyl}carbamate 174b (0.6 g, 1.7 mmol, 1.5 eq), acetylacetonato bis(ethylene) rhodium (I) (29.64 mg, 0.12 mmol, 0.1 eq) and R-BINAP (91.2 mg, 0.12 mmol, 0.1 eq) in dioxane (8.0 mL) and H₂O (0.8 mL). The reaction was stirred for 3 h at 100° C., then cooled and quenched with water (10 mL). This was extracted with EtOAc (3×15 mL). The organic layer was dried over anhydrous Na₂SO₄, filtered and the filtrate was concentrated. The residue was dissolved in DCM (3 mL) and was purified by silica gel column chromatography (2:1 Petroleum ether: AcOEt) to afford tert-butyl N-(2-{3-[(3aR,4R,6aR)-2,2-dimethyl-6-oxo-tetrahydrocyclopenta[d][1,3]dioxol-4-yl]phenyl}ethyl)carbamate 175b (0.36 g, 84%) as a white solid. LCMS (conditions XBridge C18, 50*3.0 mm, 3.5 μm, 5%-100% Acetonitrile/5 mM aqueous NH₄HCO₃-2 min, 1.50 mL/min,): T_{R}=1.44 min; ES m/z [M+H]⁺: 376.

### Tert-butyl N-(2-{3-[(3aR,4R,6S,6aS)-6-hydroxy-2,2-dimethyl-tetrahydro-3aH-cyclopenta[d][1,3]dioxol-4-yl]phenyl}ethyl)carbamate (176b)

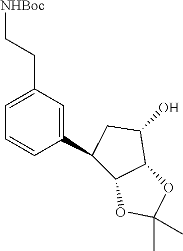 [SMILES: CC(C)(C)OC(=O)NCCc1cccc([C@H]2C[C@H](O)[C@@H]3OC(C)(C)O[C@@H]32)c1]

Into an 8 mL vial was added tert-butyl N-(2-{3-[(3aR,4R,6aR)-2,2-dimethyl-6-oxo-tetrahydrocyclopenta[d][1,3]dioxol-4-yl]phenyl}ethyl)carbamate 175b (0.36 g, 0.96 mmol, 1.0 eq) and MeOH (4.0 mL), followed by NaBH₄ (54.7 mg, 1.45 mmol, 1.5 eq) at 0° C. The reaction was stirred for 0.5 h then quenched with water (10 mL) and extracted with EtOAc (3×10 mL). The organic layer was dried over anhydrous Na₂SO₄, filtered and the filtrate was concentrated. The residue was dissolved in DCM (2 mL) and was purified by silica gel column chromatography (1:1 Petroleum ether: AcOEt) to afford tert-butyl N-(2-{3-[(3aR,4R,6S,6aS)-6-hydroxy-2,2-dimethyl-tetrahydro-3aH-cyclopenta[d][1,3]dioxol-4-yl]phenyl}ethyl)carbamate 176b (0.2 g, 56%) as a white solid. LCMS (conditions XBridge C18, 50*3.0 mm, 3.5 μm, 5%-100% Acetonitrile/5 mM aqueous NH₄HCO₃-2 min, 1.50 mL/min,): T_{R}=1.44 min; ES m/z [M+H]⁺: 378.

### Tert-butyl N-(2-{3-[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]phenyl]ethyl)carbamate (177b)

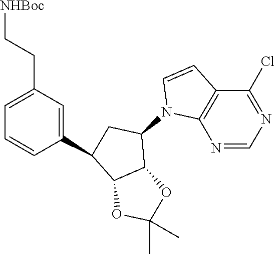 [SMILES: CC(C)(C)OC(=O)NCCc1cccc([C@H]2C[C@@H](n3ccc4c(Cl)ncnc43)[C@@H]3OC(C)(C)O[C@@H]32)c1]

Into an 8 mL vial were added tert-butyl N-(2-{3-[(3aR,4R,6S,6aS)-6-hydroxy-2,2-dimethyl-tetrahydro-3aH-cyclopenta[d][1,3]dioxol-4-yl]phenyl}ethyl)carbamate 176b (0.2 g, 0.54 mmol, 1.3 eq), 4-chloro-7H-pyrrolo[2,3-d]pyrimidine (63.2 mg, 0.41 mmol, 1.0 eq) and PPh₃ (0.22 g, 0.82 mmol, 2.0 eq) in THF (3 mL), followed by DIAD (0.17 g, 0.82 mmol, 2.0 eq) at 0° C. The resulting mixture was stirred for 12 h, then concentrated. The residue was dissolved in DCM (2 mL) and was purified by silica gel column chromatography (1:1 Petroleum ether: AcOEt) to afford tert-butyl N-(2-{3-[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]phenyl}ethyl)carbamate 177b (0.19 g, 88%) as a white solid. LCMS (conditions XBridge C18, 50*3.0 mm, 3.5 μm, 5%-100% Acetonitrile/5 mM aqueous NH₄HCO₃-2 min, 1.50 mL/min,): T_{R}=1.64 min; ES m/z [M+H]⁺: 513, 515.

### Tert-butyl N-(2-{3-[(3aR,4R,6R,6aS)-6-(4-{[(4-methoxyphenyl)methyl]amino}pyrrolo[2,3-d]pyrimidin-7-yl)-2,2-dimethyl-tetrahydro-3aH-cyclopenta[d][1,3]dioxol-4-yl]phenyl}ethyl)carbamate (178b)

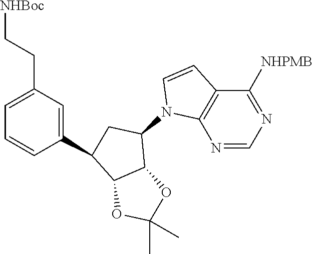

Into an 8 mL vial were added tert-butyl N-(2-{3-[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]phenyl}ethyl)carbamate 177b (0.19 g, 0.36 mmol, 1.0 eq), (4-methoxyphenyl)methanamine (0.15 g, 1.1 mmol, 3.0 eq) and TEA (73.1 mg, 0.73 mmol, 2.0 eq) in EtOH (4.0 mL). The reaction was stirred for 2 h at 80° C., then concentrated. The residue was dissolved in DCM (2 mL) and was purified by silica gel column chromatography (1:1 Petroleum ether: AcOEt) to afford tert-butyl N-(2-{3-[(3aR,4R,6R,6aS)-6-(4-{[(4-methoxyphenyl)methyl]amino}pyrrolo[2,3-d]pyrimidin-7-yl)-2,2-dimethyl-tetrahydro-3aH-cyclopenta[d][1,3]dioxol-4-yl]phenyl}ethyl)carbamate 178b (99 mg, 44%) as a white solid. LCMS (conditions XBridge C18, 50*3.0 mm, 3.5 μm, Mobile Phase A: Water/5 mM NH₄HCO₃, Mobile Phase B: Methanol, 5%-100% B-2 min, 1.50 mL/min,): T_{R}=1.55 min; ES m/z [M+H]⁺: 614.

### (1S,2R,3R,5R)-3-[3-(2-Aminoethyl)phenyl]-5-{4-aminopyrrolo[2,3-d]pyrimidin-7-yl}cyclopentane-1,2-diol (592)

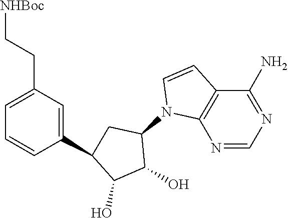 [SMILES: CC(C)(C)OC(=O)NCCc1cccc([C@H]2C[C@@H](n3ccc4c(N)ncnc43)[C@H](O)[C@@H]2O)c1]

Into an 8 mL vial were added tert-butyl N-(2-{3-[(3aR,4R,6R,6aS)-6-(4-{[(4-methoxyphenyl)methyl]amino}pyrrolo[2,3-d]pyrimidin-7-yl)-2,2-dimethyl-tetrahydro-3aH-cyclopenta[d][1,3]dioxol-4-yl]phenyl}ethyl)carbamate 178b (99 mg, 0.16 mmol, 1.0 eq) and TFA (0.5 mL) in DCM (0.5 mL). The resulting mixture was stirred for 0.5 h at 80° C., then concentrated. The residue was dissolved in MeOH (3 mL). The mixture was basified to pH >8 with NH₃·H₂O and was purified by Prep-HPLC [column, Xbridge Prep C18 OBD column, 5 μm, 19*150 mm; mobile phase, Water (0.03% NH₄OH) and CH₃CN (20-40% over 10 min); Detector, UV 220&254 nm]to afford (1S,2R,3R,5R)-3-[3-(2-aminoethyl)phenyl]-5-{4-aminopyrrolo[2,3-d]pyrimidin-7-yl}cyclopentane-1,2-diol 592 (10.4 mg, 18%) as an off-white solid. LCMS (conditions: ZORBAX SB-Aq, 50*4.6 mm, 1.8 μm, 5%-95% Acetonitrile/0.02% aqueous TFA-3 min, 1.50 mL/min): T_{R}=0.98 min; ES m/z [M+H]⁺: 354. ¹H NMR (400 MHZ, DMSO-d₆+D₂O) δ 8.07 (s, 1H), 7.36 (d, J=3.6 Hz, 1H), 7.25-7.21 (m, 3H), 7.06 (d, J=6.9 Hz, 1H), 6.57 (d, J=3.5 Hz, 1H), 4.94-4.83 (m, 1H), 4.30 (t, J=6.7 Hz, 1H), 4.06 (t, J=6.7 Hz, 1H), 3.21-3.12 (m, 2H), 3.12-3.01 (m, 1H), 2.78-2.60 (m, 2H), 2.40-2.28 (m, 1H), 2.02 (q, J=11.7 Hz, 1H).

### Synthesis of (1R,2S,3R,5R)-3-(4-amino-5-(4-benzylthiazol-2-yl)-7H-pyrrolo[2,3-d]pyrimidin-7-yl)-5-(piperidin-3-yl)cyclopentane-1,2-diol (593)

### Tert-Butyl 3-((3aR,6R,6aS)-6-(4-chloro-5-iodo-7H-pyrrolo[2,3-d]pyrimidin-7-yl)-2,2-dimethyltetrahydro-4H-cyclopenta[d][1,3]dioxol-4-yl)piperidine-1-carboxylate (179b)

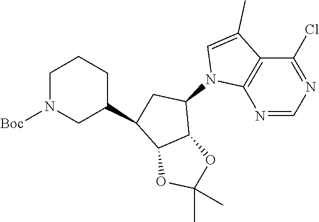 [SMILES: Cc1cn([C@@H]2C[C@H](C3CCCN(C(=O)OC(C)(C)C)C3)[C@H]3OC(C)(C)O[C@H]32)c2ncnc(Cl)c12]

Into a 40 mL vial were added tert-butyl 3-[(3aR,6S,6aS)-6-hydroxy-2,2-dimethyl-tetrahydro-3aH-cyclopenta[d][1,3]dioxol-4-yl]piperidine-1-carboxylate 114b (590 mg, 1.73 mmol, 1.0 eq), 4-chloro-5-iodo-7H-pyrrolo[2,3-d]pyrimidine (579 mg, 2.07 mmol, 1.2 eq), PPh₃ (1133 mg, 4.32 mmol, 2.5 eq), toluene (10.0 mL) and DBAD (795 mg, 3.45 mmol, 2.0 eq). The resulting mixture was stirred for 2 h at 50° C., then cooled. The reaction was quenched with water (30 mL) and then extracted with EtOAc (3×30 mL). The organic layer was dried over anhydrous Na₂SO₄, filtered and the filtrate was concentrated. The residue was purified by silica gel column chromatography (50:50 AcOEt: petroleum ether) to afford tert-butyl 3-[(3aR,6R,6aS)-6-{4-chloro-5-iodopyrrolo[2,3-d]pyrimidin-7-yl}-2,2-dimethyl-tetrahydro-3aH-cyclopenta[d][1,3]dioxol-4-yl]piperidine-1-carboxylate 179b (480 mg, 46%) as a yellow solid. LCMS [XBridge C18, 50*3.0 mm, 3.5 μm; Mobile Phase 10-95% Acetonitrile/5 mM aqueous NH₄HCO₃-3 min, 1.00 mL/min]: T_{R}=2.29 min ES m/z [M+H]⁺: 603, 605.

### tert-Butyl 3-((3aR,6R,6aS)-6-(4-amino-5-iodo-7H-pyrrolo[2,3-d]pyrimidin-7-yl)-2,2-dimethyltetrahydro-4H-cyclopenta[d][1,3]dioxol-4-yl)piperidine-1-carboxylate (180b)

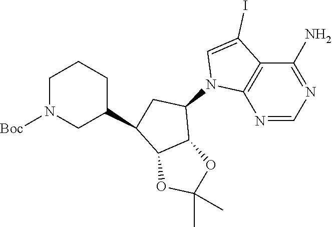 [SMILES: CC(C)(C)OC(=O)N1CCCC([C@H]2C[C@@H](n3cc(I)c4c(N)ncnc43)[C@@H]3OC(C)(C)O[C@@H]32)C1]

Into a 40 mL vial were added tert-butyl 3-[(3aR,6R,6aS)-6-{4-chloro-5-iodopyrrolo[2,3-d]pyrimidin-7-yl}-2,2-dimethyl-tetrahydro-3aH-cyclopenta[d][1,3]dioxol-4-yl]piperidine-1-carboxylate 179b (480 mg, 0.79 mmol, 1.0 eq) and 7M NH₃ in MeOH (5 mL). The resulting mixture was stirred for 16 h at 70° C., then concentrated. The residue was purified by silica gel column chromatography (50:50 AcOEt: petroleum ether) to afford tert-butyl 3-[(3aR,6R,6aS)-6-{4-amino-5-iodopyrrolo[2,3-d]pyrimidin-7-yl}-2,2-dimethyl-tetrahydro-3aH-cyclopenta[d][1,3]dioxol-4-yl]piperidine-1-carboxylate 180b (320 mg, 68%) as a yellow solid. LCMS [XBridge C18, 50*3.0 mm, 3.5 μm; Mobile Phase 10-95% Acetonitrile/5 mM aqueous NH₄HCO₃-3 min, 1.00 mL/min]: T_{R}=2.25 min; ES m/z [M+H]⁺: 584.

### tert-Butyl 3-((3aR,6R,6aS)-6-(4-amino-5-(4-benzylthiazol-2-yl)-7H-pyrrolo[2,3-d]pyrimidin-7-yl)-2,2-dimethyltetrahydro-4H-cyclopenta[d][1.3]dioxol-4-yl)piperidine-1-carboxylate (181b)

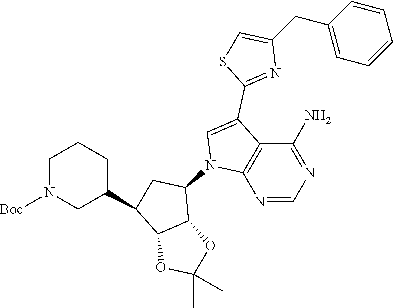 [SMILES: CC(C)(C)OC(=O)N1CCCC([C@H]2C[C@@H](n3cc(-c4nc(Cc5ccccc5)cs4)c4c(N)ncnc43)[C@@H]3OC(C)(C)O[C@@H]32)C1]

To a solution of tert-butyl 3-[(3aR,6R,6aS)-6-{4-amino-5-iodopyrrolo[2,3-d]pyrimidin-7-yl}-2,2-dimethyl-tetrahydro-3aH-cyclopenta[d][1,3]dioxol-4-yl]piperidine-1-carboxylate 179b (160 mg, 0.27 mmol, 1.0 eq) and (4-benzyl-1,3-thiazol-2-yl)(chloro) zinc (151 mg, 0.55 mmol, 2.0 eq) in toluene (4.0 mL) were added Pd (PPh₃) 2Cl₂ (38 mg, 0.05 mmol, 0.2 eq) and K₃PO₄ (174 mg, 0.82 mmol, 3.0 eq). After stirring for 18 h at 60° C. under a nitrogen atmosphere, the resulting mixture was concentrated, then extracted with EtOAc (3×10 mL). The organic layer was dried over anhydrous Na₂SO₄, filtered and the filtrate was concentrated. The residue was purified by silica gel column chromatography (50:50 AcOEt: petroleum ether) to afford tert-butyl 3-[(3aR,6R,6aS)-6-[4-amino-5-(4-benzyl-1,3-thiazol-2-yl)pyrrolo[2,3-d]pyrimidin-7-yl]-2,2-dimethyl-tetrahydro-3aH-cyclopenta[d][1,3]dioxol-4-yl]piperidine-1-carboxylate 180b (85 mg, 49%) as a yellow solid. LCMS [XBridge C18, 50*3.0 mm, 3.5 μm; Mobile Phase 10-95% Acetonitrile/5 mM aqueous NH₄HCO₃-3 min, 1.00 mL/min]: T_{R}=2.41 min; ES m/z [M+H]⁺: 631.

### (1R,2S,3R,5R)-3-(4-Amino-5-(4-benzylthiazol-2-yl)-7H-pyrrolo[2,3-d]pyrimidin-7-yl)-5-(piperidin-3-yl)cyclopentane-1,2-diol (593)

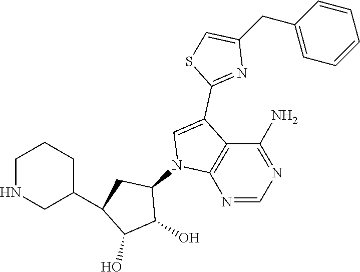 [SMILES: Nc1ncnc2c1c(-c1nc(Cc3ccccc3)cs1)cn2[C@@H]1C[C@H](C2CCCNC2)[C@@H](O)[C@H]1O]

Into an 8 mL vial were added tert-butyl 3-[(3aR,6R,6aS)-6-[4-amino-5-(4-benzyl-1,3-thiazol-2-yl)pyrrolo[2,3-d]pyrimidin-7-yl]-2,2-dimethyl-tetrahydro-3aH-cyclopenta[d][1,3]dioxol-4-yl]piperidine-1-carboxylate 180b (85 mg, 0.13 mmol, 1.0 eq) and 4M HCl in MeOH (2 mL). The resulting mixture was stirred for 1 h at 50° C., then concentrated. The mixture was basified to pH>8 with saturated Na₂CO₃ (aq.). The crude product was purified by Prep-HPLC [Column: YMC-Actus Triart C18 ExRS, 30*150 mm, 5 μm; Mobile Phase A: Water (10 mmol/L NH₄HCO₃+0.1% NH₃·H₂O), Mobile Phase B: ACETONITRILE; Flow rate: 30 mL/min; Gradient: 20% B to 58% B over 12 min; Detector, UV 220&254 nm]to afford (1R,2S,3R,5R)-3-[4-amino-5-(4-benzyl-1,3-thiazol-2-yl)pyrrolo[2,3-d]pyrimidin-7-yl]-5-(piperidin-3-yl)cyclopentane-1,2-diol 593 (45 mg, 67%) as an off-white solid. ¹H NMR (400 MHZ, DMSO-d₆+H₂O) δ 8.07 (s, 1H), 8.04 (s, 1H), 7.42-7.25 (m, 4H), 7.25-7.14 (m, 2H), 4.83-4.73 (m, 1H), 4.23-4.14 (m, 1H), 4.04 (s, 2H), 3.79-3.73 (m, 1H), 3.17-2.76 (m, 2H), 2.43-2.32 (m, 1H), 2.25-2.10 (m, 1H), 2.02-1.98 (m, 1H), 1.73-1.50 (m, 4H), 1.43-1.38 (m, 2H), 1.05-0.92 (m, 1H). LCMS [ZORBAX SB-Aq, 50*4.6 mm, 1.8 μm; Mobile Phase 5-95% Acetonitrile/0.02% aqueous TFA-3 min, 1.50 mL/min]: T_{R}=1.23, 1.26 min; ES m/z [M+H]⁺: 491.

### (1R,2S,3R,5R)-3-(4-Amino-5-(4-benzylthiazol-2-yl)-7H-pyrrolo[2,3-d]pyrimidin-7-yl)-5-((R)-piperidin-3-yl)cyclopentane-1,2-diol (593R, structure assumed) and (1R,2S,3R,5R)-3-(4-amino-5-(4-benzylthiazol-2-yl)-7H-pyrrolo[2,3-d]pyrimidin-7-yl)-5-((S)-piperidin-3-yl)cyclopentane-1,2-diol (593S, structure assumed)

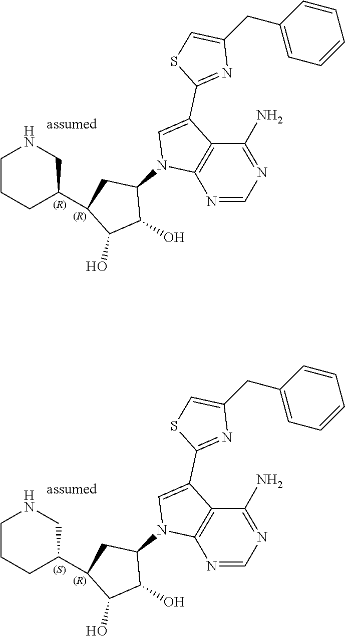

(1R,2S,3R,5R)-3-[4-Amino-5-(4-benzyl-1,3-thiazol-2-yl)pyrrolo[2,3-d]pyrimidin-7-yl]-5-(piperidin-3-yl)cyclopentane-1,2-diol 593 (45 mg, 0.09 mmol, 1.0 eq) was purified by Prep-HPLC [Column: YMC-Actus Triart C18 ExRS, 30*150 mm, 5 μm; Mobile Phase A: Water (10 mmol/L NH₄HCO₃+0.1% NH₃·H₂O), Mobile Phase B: ACETONITRILE; Flow rate: 30 mL/min; Gradient: 20% B to 58% B over 12 min; Detector, UV 220&254 nm]to afford (1R,2S,3R,5R)-3-(4-amino-5-(4-benzylthiazol-2-yl)-7H-pyrrolo[2,3-d]pyrimidin-7-yl)-5-((R)-piperidin-3-yl)cyclopentane-1,2-diol 593R (15 mg, 33%, structure assumed) as an off-white solid. ¹H NMR (400 MHZ, DMSO-d₆+H₂O) δ 8.06 (s, 1H), 8.05 (s, 1H), 7.36-7.26 (m, 4H), 7.26-7.14 (m, 2H), 4.83-4.72 (m, 1H), 4.24-4.16 (m, 1H), 4.04 (s, 2H), 3.82-3.75 (m, 1H), 2.92-2.85 (m, 2H), 2.47-2.40 (m, 1H), 2.29-2.18 (m, 1H), 2.06-1.91 (m, 2H), 1.66-1.62 (m, 3H), 1.65-1.50 (m, 2H), 1.24-1.06 (m, 1H). LCMS [ZORBAX SB-Aq, 50*4.6 mm, 1.8 μm; Mobile Phase 5-95% Acetonitrile/0.02% aqueous TFA-3 min, 1.50 mL/min]: T_{R}=1.22 min; ES m/z [M+H]⁺: 491.

And (1R,2S,3R,5R)-3-(4-amino-5-(4-benzylthiazol-2-yl)-7H-pyrrolo[2,3-d]pyrimidin-7-yl)-5-((S)-piperidin-3-yl)cyclopentane-1,2-diol 593S (15 mg, 33%, structure assumed) as an off-white solid. ¹H NMR (400 MHZ, DMSO-d₆+H₂O) δ 8.08 (s, 1H), 8.05 (s, 1H), 7.36-7.26 (m, 4H), 7.24-7.17 (m, 2H), 4.83-4.73 (m, 1H), 4.23-4.15 (m, 1H), 4.04 (s, 2H), 3.79-3.72 (m, 1H), 3.15-3.08 (m, 1H), 2.88-2.80 (m, 1H), 2.43-2.33 (m, 1H), 2.26-2.16 (m, 1H), 2.03-1.98 (m, 1H), 1.73-1.50 (m, 4H), 1.44-1.39 (m, 2H), 1.06-0.92 (m, 1H). LCMS [ZORBAX SB-Aq, 50*4.6 mm, 1.8 μm; Mobile Phase 5-95% Acetonitrile/0.02% aqueous TFA-3 min, 1.50 mL/min]: T_{R}=1.26 min; ES m/z [M+H]⁺: 491.

### Synthesis of (1R,2S,3R,5R)-3-(4-amino-2-chloro-7H-pyrrolo[2,3-d]pyrimidin-7-yl)-5-(3-(isothiazol-4-yl)phenyl)cyclopentane-1,2-diol (594)

### (3aR,6R,6aR)-6-(3-(Isothiazol-4-yl)phenyl)-2,2-dimethyltetrahydro-4H-cyclopenta[d][1,3]dioxol-4-one (185b)

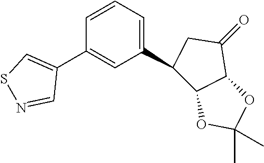 [SMILES: CC1(C)O[C@@H]2[C@@H](c3cccc(-c4cnsc4)c3)CC(=O)[C@@H]2O1]

To a solution of (3aR,6R,6aR)-6-(3-bromophenyl)-2,2-dimethyl-tetrahydrocyclopenta[d][1,3]dioxol-4-one (0.5 g, 1.61 mmol, 1.0 eq) and 4-(4,4,5,5-tetramethyl-1,3,2-dioxaborolan-2-yl)-1,2-thiazole (0.68 g, 3.21 mmol, 2.0 eq) in dioxane (5 mL) and H₂O (0.5 mL) were added K₂CO₃ (0.67 g, 4.82 mmol, 3.0 eq) and Pd (dppf) Cl₂·CH₂Cl₂ (0.13 g, 0.16 mmol, 0.1 eq). After stirring for 2 h at 80° C. under a nitrogen atmosphere, the resulting mixture was concentrated and water was added. The resulting mixture was extracted with ethyl acetate (3×30 mL). The combined organic layers were washed with brine and dried over anhydrous Na₂SO₄ and concentrated. The residue was purified by silica gel column chromatography (3:2 petroleum ether:ethyl acetate) to afford (3aR,6R,6aR)-2,2-dimethyl-6-[3-(1,2-thiazol-4-yl)phenyl]-tetrahydrocyclopenta[d][1,3]dioxol-4-one 185b (280 mg, 55%) as a colorless oil. LCMS (conditions: L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min), T_{R}=1.674 min; ES m/z [M+H]⁺: 316.

### (3aS,45,6R,6aR)-6-(3-(Isothiazol-4-yl)phenyl)-2,2-dimethyltetrahydro-4H-cyclopenta[d][1,3]dioxol-4-ol (186b)

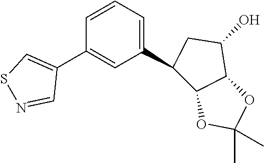 [SMILES: CC1(C)O[C@@H]2[C@H](O1)[C@@H](c1cccc(-c3cnsc3)c1)C[C@@H]2O]

To (3aR,6R,6aR)-2,2-dimethyl-6-[3-(1,2-thiazol-4-yl)phenyl]-tetrahydrocyclopenta[d][1,3]dioxol-4-one 185b (0.26 g, 0.82 mmol, 1.0 eq) in MeOH (3 mL) was added NaBH₄ (62.4 mg, 1.65 mmol, 2.0 eq), and the reaction was stirred for 0.5h at 0° C. The mixture was quenched by addition of ammonium chloride solution, and extracted with ethyl acetate (3×30 mL). The combined organic phase was washed with brine, dried over anhydrous sodium sulfate and concentrated. The residue was purified by silica gel column chromatography (1:1 petroleum ether:ethyl acetate) to afford (3aS,45,6R,6aR)-2,2-dimethyl-6-[3-(1,2-thiazol-4-yl)phenyl]-tetrahydro-3aH-cyclopenta[d][1,3]dioxol-4-ol 186b (0.12 g, 46%) as a colorless oil. LCMS (conditions: Cortecs C18+, 50*3.0 mm, 2.7 μm, Mobile Phase A: Water/0.05% TFA, Mobile Phase B: Acetonitrile, 2%-100% B-3 min), T_{R}=1.36 min; ES m/z [M+H]⁺: 318.

### 4-(3-((3aR,4R,6R,6aS)-6-(2,4-Dichloro-7H-pyrrolo[2,3-d]pyrimidin-7-yl)-2,2-dimethyltetrahydro-4H-cyclopenta[d][1,3]dioxol-4-yl)phenyl) isothiazole (187b)

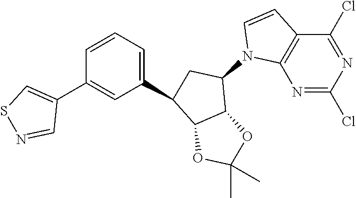 [SMILES: CC1(C)O[C@@H]2[C@H](O1)[C@@H](c1cccc(-c3cnsc3)c1)C[C@H]2n1ccc2c(Cl)nc(Cl)nc21]

To a stirred mixture of (3aS,4S,6R,6aR)-2,2-dimethyl-6-[3-(1,2-thiazol-4-yl)phenyl]-tetrahydro-3aH-cyclopenta[d][1,3]dioxol-4-ol 186b (0.11 g, 0.34 mmol, 1.2 eq), PPh₃ (0.15 g, 0.57 mmol, 2.0 eq) and 2,4-dichloro-7H-pyrrolo[2,3-d]pyrimidine (54 mg, 0.29 mmol, 1.0 eq) in THF (1.5 mL) was added DIAD (0.12 g, 0.57 mmol, 2.0 eq) in THF (0.5 mL) dropwise at 0° C. The resulting mixture was stirred for 5h then concentrated. The residue was suspended in water and extracted with ethyl acetate (3×10 mL). The combined organic phase was washed with brine, dried over anhydrous Na₂SO₄ and concentrated. The residue was purified by column chromatography (1:3 tetrahydrofuran: petroleum ether) to afford 4-{3-[(3aR,4R,6R,6aS)-6-{2,4-dichloropyrrolo[2,3-d]pyrimidin-7-yl}-2,2-dimethyl-tetrahydro-3aH-cyclopenta[d][1,3]dioxol-4-yl]phenyl}-1,2-thiazole 187b (0.11 g, 78%) as a colorless oil. LCMS (conditions: Cortecs C18+, 50*3.0 mm, 2.7 μm, Mobile Phase A: Water/0.05% TFA, Mobile Phase B: Acetonitrile, 2%-100% B-3 min), T_{R}=1.878 min; ES m/z [M+H]⁺: 487, 489.

### 2-Chloro-7-((3aS,4R,6R,6aR)-6-(3-(isothiazol-4-yl)phenyl)-2,2-dimethyltetrahydro-4H-cyclopenta[d][1,3]dioxol-4-yl)-N-(4-methoxybenzyl)-7H-pyrrolo [2,3-d]pyrimidin-4-amine (188b)

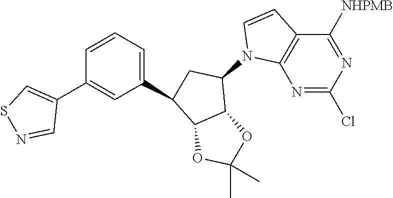

A mixture of 4-{3-[(3aR,4R,6R,6aS)-6-{2,4-dichloropyrrolo[2,3-d]pyrimidin-7-yl}-2,2-dimethyl-tetrahydro-3aH-cyclopenta[d][1,3]dioxol-4-yl]phenyl}-1,2-thiazole 187b (0.11 g, 0.23 mmol, 1.0 eq), TEA (68.5 mg, 0.68 mmol, 3.0 eq) and 4-methoxy-benzenemethanamine, (61.9 mg, 0.45 mmol, 2.0 eq) in EtOH (1.5 mL) was stirred for 4 h at 80° C., then concentrated. The residue was purified by silica gel column chromatography (1:1 ethyl acetate:petroleum ether) to afford 7-[(3aS,4R,6R,6aR)-2,2-dimethyl-6-[3-(1,2-thiazol-4-yl)phenyl]-tetrahydro-3aH-cyclopenta[d][1,3]dioxol-4-yl]-2-chloro-N-[(4-methoxyphenyl)methyl]pyrrolo[2,3-d]pyrimidin-4-amine 188 (70 mg, 52%) as a colorless oil. LCMS (conditions: L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min), T_{R}=1.716 min; ES m/z [M+H]⁺: 588. 590.

### (1R,2S,3R,5R)-3-(4-Amino-2-chloro-7H-pyrrolo[2,3-d]pyrimidin-7-yl)-5-(3-(isothiazol-4-yl)phenyl)cyclopentane-1,2-diol (594)

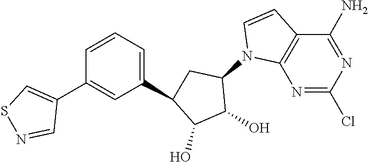 [SMILES: Nc1nc(Cl)nc2c1ccn2[C@@H]1C[C@H](c2cccc(-c3cnsc3)c2)[C@@H](O)[C@H]1O]

Into an 8 mL vial were added 7-[(3aS,4R,6R,6aR)-2,2-dimethyl-6-[3-(1,2-thiazol-4-yl)phenyl]-tetrahydro-3aH-cyclopenta[d][1,3]dioxol-4-yl]-2-chloro-N-[(4-methoxyphenyl)methyl]pyrrolo[2,3-d]pyrimidin-4-amine 188b (70 mg, 0.12 mmol, 1.0 eq) and TFA (2 mL). The resulting mixture was stirred for 0.5h at 60° C., then concentrated and basified to pH>8 with NH₃·H₂O. The mixture was purified by Prep-HPLC with the following conditions (Column: Column: YMC-Actus Triart C18, 30*150 mm, 5 μm; Mobile Phase A: Water (10 mmol/L NH₄HCO₃+0.1% NH₃·H₂O), Mobile Phase B: ACETONITRILE; Flow rate: 30 mL/min; Gradient: 20% B to 55% B over 9 min) to afford (1R,2S,3R,5R)-3-{4-amino-2-chloropyrrolo[2,3-d]pyrimidin-7-yl}-5-[3-(1,2-thiazol-4-yl)phenyl]cyclopentane-1,2-diol 594 (10.9 mg, 21%) as a white solid. LCMS (Cortecs C18+, 50*3.0 mm, 2.7 μm, Mobile Phase A: Water/0.02% TFA, Mobile Phase B: Acetonitrile, 5%-95% B-3 min), T_{R}=1.22 min; ES m/z [M+H]⁺: 428, 430. ¹H NMR (400 MHZ, DMSO-d₆) δ 9.36 (s, 1H), 9.08 (s, 1H), 7.77 (s, 1H), 7.69-7.61 (m, 1H), 7.48-7.40 (m, 5H), 6.61 (d, J=3.6 Hz, 1H), 4.96 (t, J=6.3 Hz, 2H), 4.92-4.81 (m, 1H), 4.27 (q, J=6.4 Hz, 1H), 4.13 (q, J=6.5 Hz, 1H), 3.23-3.12 (m, 1H), 2.45-2.35 (m, 1H), 2.03 (q, J=11.9 Hz, 1H).

### Synthesis of (1R,2S,3R,5R)-3-[4-amino-5-(4-benzyl-1,3-thiazol-2-yl)pyrrolo[2,3-d]pyrimidin-7-yl]-5-(3-methylpiperidin-4-yl)cyclopentane-1,2-diol (595)

### 1-(Tert-butoxycarbonyl)-3-methyl-3,6-dihydro-2H-pyridin-4-ylboronic acid (189b)

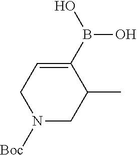 [SMILES: CC1CN(C(=O)OC(C)(C)C)CC=C1B(O)O]

Into a 250 ml 3-necked round-bottom flask were added tert-butyl 3-methyl-4-(4,4,5,5-tetramethyl-1,3,2-dioxaborolan-2-yl)-3,6-dihydro-2H-pyridine-1-carboxylate (8 g, 24.8 mmol, 1.0 eq) [WO2011025006], NalO₄ (15.9 g, 74.3 mmol, 3.0 eq) and NH₄OAc (5.7 g, 74.3 mmol, 3.0 eq) in H₂O (40.0 mL) and acetone (40.0 mL). The reaction was stirred for 12 h, then concentrated. The resulting mixture was diluted with MeOH (40.0 mL). The residue was purified by Prep-HPLC [conditions XBridge C18, 50*3.0 mm, 3.5 μm, Mobile Phase A: 0.05% tetrahydrofuran, Mobile Phase B: Acetonitrile, 5%-100% B-2 min, 1.50 mL/min,); Detector, UV 220&254 nm]to afford 1-(tert-butoxycarbonyl)-3-methyl-3,6-dihydro-2H-pyridin-4-ylboronic acid 189b (1.8 g, 30%) as a white solid. LCMS (conditions Cortecs C18, 50*3.0 mm, 2.7 μm, Mobile Phase A: Water/0.05% TFA, Mobile Phase B: Acetonitrile, 2%-100% B-2 min, 1.50 mL/min): T_{R}=0.84 min; ES m/z [M+H]⁺: 242.

### Tert-butyl 4-[(3aR,4R,6aR)-2,2-dimethyl-6-oxo-tetrahydrocyclopenta[d][1,3]dioxol-4-yl]-3-methyl-3,6-dihydro-2H-pyridine-1-carboxylate (190b)

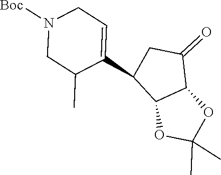 [SMILES: CC1CN(C(=O)OC(C)(C)C)CC=C1[C@H]1CC(=O)[C@@H]2OC(C)(C)O[C@H]12]

Into a 250 ml flask were added 1-(tert-butoxycarbonyl)-3-methyl-3,6-dihydro-2H-pyridin-4-ylboronic acid 189b (1.4 g, 5.9 mmol, 1.2 eq), (3aR,6aR)-2,2-dimethyl-3aH,6aH-cyclopenta[d][1,3]dioxol-4-one (0.76 g, 4.9 mmol, 1.0 eq), [Rh(COD)Cl]2 (0.24 g, 0.5 mmol, 0.1 eq) and 2M KOH (312 μL, 5.6 mmol, 1.13 eq) in MeOH (100 mL). The reaction mixture was irradiated with microwave radiation for 2 h at 40° C., then quenched with water (20 mL) and extracted with EtOAc (3×30 mL). The organic layer was dried over anhydrous Na₂SO₄, filtered and the filtrate was concentrated. The residue was dissolved in DCM (10 mL) and was purified by silica gel column chromatography (3:1 Petroleum ether: AcOEt) to afford tert-butyl 4-[(3aR,4R,6aR)-2,2-dimethyl-6-oxo-tetrahydrocyclopenta[d][1,3]dioxol-4-yl]-3-methyl-3,6-dihydro-2H-pyridine-1-carboxylate 190b (0.92 g, 53%) as a white solid. LCMS (conditions XBridge C18, 50*3.0 mm, 3.5 μm, Mobile Phase A: Water/5 mM NH₄HCO₃, Mobile Phase B: Methanol, 10%-95% B-2 min, 1.50 mL/min): T_{R}=1.50 min; ES m/z [M+H]⁺: 352.

### Tert-butyl 4-[(3aR,4R,6aR)-2,2-dimethyl-6-oxo-tetrahydrocyclopenta[d][1,3]dioxol-4-yl]-3-methylpiperidine-1-carboxylate (191b)

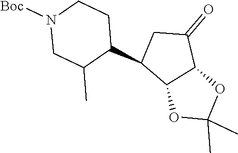 [SMILES: CC1CN(C(=O)OC(C)(C)C)CCC1[C@H]1CC(=O)[C@@H]2OC(C)(C)O[C@H]12]

To a solution of tert-butyl 4-[(3aR,4R,6aR)-2,2-dimethyl-6-oxo-tetrahydrocyclopenta[d][1,3]dioxol-4-yl]-3-methyl-3,6-dihydro-2H-pyridine-1-carboxylate 190b (0.92 g, 2.6 mmol, 1.0 eq) in MeOH (10 mL) in a 40 mL vial was added 10% Pd/C (0.1 g). The mixture was hydrogenated at room temperature for 12 h under a hydrogen balloon, then filtered through a Celite pad and concentrated to afford tert-butyl 4-[(3aR,4R,6aR)-2,2-dimethyl-6-oxo-tetrahydrocyclopenta[d][1,3]dioxol-4-yl]-3-methylpiperidine-1-carboxylate 191b (0.9 g, 97%) as a white solid. LCMS (conditions XBridge C18, 50*3.0 mm, 3.5 μm, Mobile Phase A: Water/5 mM NH₄HCO₃, Mobile Phase B: Methanol, 10%-95% B-2 min, 1.50 mL/min): T_{R}=1.48 min; ES m/z [M+H]⁺: 354.

### Tert-butyl 4-[(3aR,4R,6S,6aS)-6-hydroxy-2,2-dimethyl-tetrahydro-3aH-cyclopenta[d][1,3]dioxol-4-yl]-3-methylpiperidine-1-carboxylate (102h)

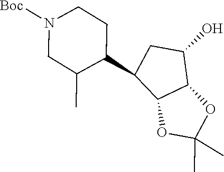 [SMILES: CC1CN(C(=O)OC(C)(C)C)CCC1[C@H]1C[C@H](O)[C@@H]2OC(C)(C)O[C@@H]21]

Into a 40 mL vial were added tert-butyl 4-[(3aR,4R,6aR)-2,2-dimethyl-6-oxo-tetrahydrocyclopenta[d][1,3]dioxol-4-yl]-3-methylpiperidine-1-carboxylate 191b (0.9 g, 2.6 mmol, 1.0 eq) and NaBH₄ (0.14 g, 3.8 mmol, 1.5 eq) in MeOH (10.0 mL) at 0° C. The resulting mixture was stirred for 0.5 h at room temperature. The reaction was quenched with water (10 mL) and then extracted with EtOAc (3×10 mL). The organic layer was dried over anhydrous Na₂SO₄, filtered and the filtrate was concentrated. The residue was dissolved in DCM (5 mL) and was purified by silica gel column chromatography (2:1 Petroleum ether: AcOEt) to afford tert-butyl 4-[(3aR,4R,6S,6aS)-6-hydroxy-2,2-dimethyl-tetrahydro-3aH-cyclopenta[d][1,3]dioxol-4-yl]-3-methylpiperidine-1-carboxylate 192b (0.7 g, 77%) as a white solid. LCMS (conditions XBridge C18, 50*3.0 mm, 3.5 μm, Mobile Phase A: Water/5 mM NH₄HCO₃, Mobile Phase B: Methanol, 10%-95% B-2 min, 1.50 mL/min): T_{R}=1.48 min; ES m/z [M+H]⁺: 356.

### Tert-butyl 4-[(3aR,4R,6R,6aS)-6-{4-chloro-5-iodopyrrolo[2,3-d]pyrimidin-7-yl}-2,2-dimethyl-tetrahydro-3aH-cyclopenta[d][1,3]dioxol-4-yl]-3-methylpiperidine-1-carboxylate (193b)

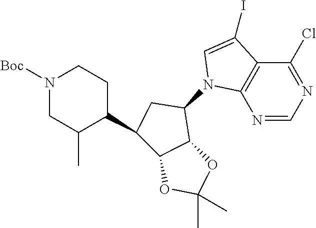 [SMILES: CC1CN(C(=O)OC(C)(C)C)CCC1[C@H]1C[C@@H](n2cc(I)c3c(Cl)ncnc32)[C@@H]2OC(C)(C)O[C@@H]21]

Into a 40 mL vial were added tert-butyl 4-[(3aR,4R,6S,6aS)-6-hydroxy-2,2-dimethyl-tetrahydro-3aH-cyclopenta[d][1,3]dioxol-4-yl]-3-methylpiperidine-1-carboxylate 192b (0.7 g, 1.9 mmol, 1.3 eq), 4-chloro-5-iodo-7H-pyrrolo[2,3-d]pyrimidine (0.42 g, 1.5 mmol, 1.0 eq), PPh₃ (0.79 g, 3.0 mmol, 2.0 eq) and toluene (10 mL). To this was added DBAD (0.7 g, 3.0 mmol, 2.0 eq) at 0° C. The reaction was stirred for 8 h at 50° C., then concentrated. The residue was dissolved in DCM (3 mL) and was purified by silica gel column chromatography (3:1 Petroleum ether: AcOEt) to afford tert-butyl 4-[(3aR,4R,6R,6aS)-6-{4-chloro-5-iodopyrrolo[2,3-d]pyrimidin-7-yl}-2,2-dimethyl-tetrahydro-3aH-cyclopenta[d][1,3]dioxol-4-yl]-3-methylpiperidine-1-carboxylate 193b (0.38 g, 41%) as a white solid. LCMS (conditions XBridge C18, 50*3.0 mm, 3.5 μm, Mobile Phase A: Water/5 mM NH₄HCO₃, Mobile Phase B: Methanol, 10%-95% B-2 min, 1.50 mL/min): T_{R}=1.74 min; ES m/z [M+H]⁺: 617, 518.

### Tert-butyl 4-[(3aR,4R,6R,6aS)-6-{4-amino-5-iodopyrrolo[2,3-d]pyrimidin-7-yl}-2,2-dimethyl-tetrahydro-3aH-cyclopenta[d][1,3]dioxol-4-yl]-3-methylpiperidine-1-carboxylate (194b)

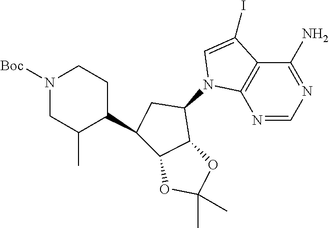 [SMILES: CC1CN(C(=O)OC(C)(C)C)CCC1[C@H]1C[C@@H](n2cc(I)c3c(N)ncnc32)[C@@H]2OC(C)(C)O[C@@H]21]

Into a 40 mL vial were added tert-butyl 4-[(3aR,4R,6R,6aS)-6-{4-chloro-5-iodopyrrolo[2,3-d]pyrimidin-7-yl}-2,2-dimethyl-tetrahydro-3aH-cyclopenta[d][1,3]dioxol-4-yl]-3-methylpiperidine-1-carboxylate 193b (0.38 g, 0.6 mmol, 1.0 eq) and 7M NH₃ in MeOH (4.0 mL). The resulting mixture was stirred for 6 h at 80° C., then concentrated. The residue was dissolved in DCM (2 mL) and was purified by silica gel column chromatography (1:1 Petroleum ether: AcOEt) to afford tert-butyl 4-[(3aR,4R,6R,6aS)-6-{4-amino-5-iodopyrrolo[2,3-d]pyrimidin-7-yl}-2,2-dimethyl-tetrahydro-3aH-cyclopenta[d][1,3]dioxol-4-yl]-3-methylpiperidine-1-carboxylate 194b (0.11 g, 30%) as a white solid. LCMS (conditions XBridge C18, 50*3.0 mm, 3.5 μm, Mobile Phase A: Water/5 mM NH₄HCO₃, Mobile Phase B: Methanol, 10%-95% B-2 min, 1.50 mL/min): T_{R}=1.60 min; ES m/z [M+H]⁺: 598.

### Tert-butyl 4-[(3aR,4R,6R,6aS)-6-[4-amino-5-(4-benzyl-1,3-thiazol-2-yl)pyrrolo[2,3-d]pyrimidin-7-yl]-2,2-dimethyl-tetrahydro-3aH-cyclopenta[d][1,3]dioxol-4-yl]-3-methylpiperidine-1-carboxylate (195b)

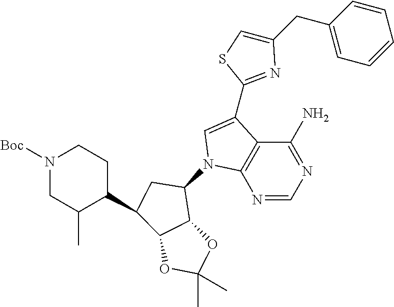 [SMILES: CC1CN(C(=O)OC(C)(C)C)CCC1[C@H]1C[C@@H](n2cc(-c3nc(Cc4ccccc4)cs3)c3c(N)ncnc32)[C@@H]2OC(C)(C)O[C@@H]21]

A solution of tert-butyl 4-[(3aR,4R,6R,6aS)-6-{4-amino-5-iodopyrrolo[2,3-d]pyrimidin-7-yl}-2,2-dimethyl-tetrahydro-3aH-cyclopenta[d][1,3]dioxol-4-yl]-3-methylpiperidine-1-carboxylate 194b (0.11 g, 0.2 mmol, 1.0 eq), Pd (PPh₃) 2Cl₂ (12.9 mg, 0.02 mmol, 0.1 eq) and (4-benzyl-1,3-thiazol-2-yl)(chloro) zinc (0.6 mmol, 3.0 eq) in toluene (3 mL) was stirred for 8 h at 60° C. under nitrogen atmosphere. The reaction was quenched with water (8 mL) and then extracted with EtOAc (3×10 mL). The organic layer was dried over anhydrous Na₂SO₄, filtered and the filtrate was concentrated. The residue was dissolved in DCM (2 mL) and was purified by silica gel column chromatography (3:2 Petroleum ether: AcOEt) to afford tert-butyl 4-[(3aR,4R,6R,6aS)-6-[4-amino-5-(4-benzyl-1,3-thiazol-2-yl)pyrrolo[2,3-d]pyrimidin-7-yl]-2,2-dimethyl-tetrahydro-3aH-cyclopenta[d][1,3]dioxol-4-yl]-3-methylpiperidine-1-carboxylate 195b (72 mg, 61%) as a white solid. LCMS (conditions XBridge C18, 50*3.0 mm, 3.5 μm, Mobile Phase A: Water/5 mM NH₄HCO₃, Mobile Phase B: Methanol, 10%-95% B-2 min, 1.50 mL/min): T_{R}=1.09 min; ES m/z [M+H]⁺: 645.

### (1R,2S,3R,5R)-3-[4-Amino-5-(4-benzyl-1,3-thiazol-2-yl)pyrrolo[2,3-d]pyrimidin-7-yl]-5-(3-methylpiperidin-4-yl)cyclopentane-12-diol (595)

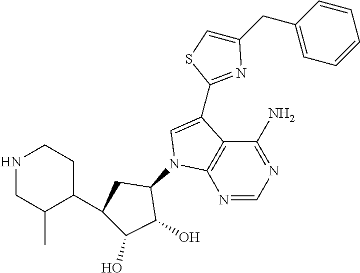 [SMILES: CC1CNCCC1[C@H]1C[C@@H](n2cc(-c3nc(Cc4ccccc4)cs3)c3c(N)ncnc32)[C@H](O)[C@@H]1O]

Into an 8 mL vial were added tert-butyl 4-[(3aR,4R,6R,6aS)-6-[4-amino-5-(4-benzyl-1,3-thiazol-2-yl)pyrrolo[2,3-d]pyrimidin-7-yl]-2,2-dimethyl-tetrahydro-3aH-cyclopenta[d][1,3]dioxol-4-yl]-3-methylpiperidine-1-carboxylate 195b (72 mg, 0.1 mmol, 1.0 eq) and 4M HCl in MeOH (2 mL). The resulting mixture was stirred for 0.5h at 60° C., then concentrated. The residue was diluted with MeOH (2 mL) and basified to pH>8 with NH₃·H₂O. The mixture was purified by Prep-HPLC (Column: YMC-Actus Triart C18, 30*150 mm, 5 μm; Mobile Phase A: aqueous 10 mmol/L NH₄HCO_{3 +0.1}% NH₃·H₂O, Mobile Phase B: ACETONITRILE; Flow rate: 30 mL/min; Gradient: 20-55% B over 8 min.) to afford (1R,2S,3R,5R)-3-[4-amino-5-(4-benzyl-1,3-thiazol-2-yl)pyrrolo[2,3-d]pyrimidin-7-yl]-5-(3-methylpiperidin-4-yl)cyclopentane-1,2-diol 595 (8.5 mg, 15%) as an off-white solid. LCMS (conditions: ZORBAX SB-Aq, 50*4.6 mm, 1.8 μm, Mobile Phase A: Water/0.02% TFA, Mobile Phase B: Acetonitrile, 5%-95% B-3 min-1.50 mL/min): T_{R}=1.27, 1.31 min; ES m/z [M+H]⁺: 505. ¹H NMR (300 MHZ, DMSO-d₆+D₂O) δ 8.03 (s, 1H), 7.97-7.86 (m, 1H), 7.36-7.25 (m, 4H), 7.29-7.15 (m, 1H), 7.12 (s, 1H), 4.82-4.73 (m, 1H), 4.30-4.10 (m, 1H), 4.05 (s, 2H), 3.90-3.77 (m, 1H), 3.00-2.60 (m, 2H), 2.46-2.20 (m, 2H), 2.21-1.86 (m, 2H), 1.82-1.08 (m, 4H), 1.04-0.76 (m, 4H).

### Synthesis of (1R,2,3R,5R)-3-(2-chloro-4-(methylamino)-7H-pyrrolo[2,3-d]pyrimidin-7-yl)-5-phenylcyclopentane-1,2-diol (596)

### 2,4-Dichloro-7-((3aS,4R,6R,6aR)-2,2-dimethyl-6-phenyltetrahydro-4H-cyclopenta[d][1,3]dioxol-4-yl)-7H-pyrrolo[2,3-d]pyrimidine (196b)

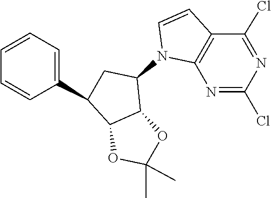 [SMILES: CC1(C)O[C@@H]2[C@H](O1)[C@@H](c1ccccc1)C[C@H]2n1ccc2c(Cl)nc(Cl)nc21]

Into a 40 mL vial was added a solution of (3aS,4S,6R,6aR)-2,2-dimethyl-6-phenyl-tetrahydro-3aH-cyclopenta[d][1,3]dioxol-4-ol (259 mg, 1.1 mmol, 1.0 eq) in THF (7.0 mL). 2,4-Dichloro-7H-pyrrolo[2,3-d]pyrimidine (208 mg, 1.1 mmol, 1.0 eq) and PPh₃ (580.36 mg, 2.2 mmol, 2.0 eq) were added, followed by DIAD (447.42 mg, 2.2 mmol, 2.0 eq) in THF (1.0 mL) in portions at 0° C. The resulting mixture was stirred for 12 h at room temperature, then concentrated. The residue was dissolved in DCM (3 mL) and was purified by flash chromatography (70:30 petroleum ether/THF) to afford 7-[(3aS,4R,6R,6aR)-2,2-dimethyl-6-phenyl-tetrahydro-3aH-cyclopenta[d][1,3]dioxol-4-yl]-2,4-dichloropyrrolo[2,3-d]pyrimidine 196b (140 mg, 31%) as a yellow oil. LCMS (conditions: Cortecs C18+, 50*3.0 mm, 2.7 μm, Mobile Phase A: Water/0.05% TFA, Mobile Phase B: Acetonitrile, 2%-95% B-3 min, 1.50 mL/min): T_{R}=1.90 min; ES m/z [M+H]⁺: 404, 406.

### 2-Chloro-7-((3aS,4R,6R,6aR)-2,2-dimethyl-6-phenyltetrahydro-4H-cyclopenta[d][1,3]dioxol-4-yl)-N-methyl-7H-pyrrolo[2,3-d]pyrimidin-4-amine (197h)

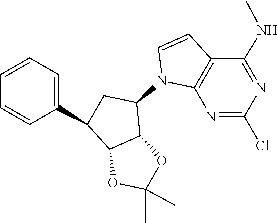 [SMILES: CNc1nc(Cl)nc2c1ccn2[C@@H]1C[C@H](c2ccccc2)[C@H]2OC(C)(C)O[C@H]21]

Into a 40 mL vial was added a solution of 7-[(3aS,4R,6R,6aR)-2,2-dimethyl-6-phenyl-tetrahydro-3aH-cyclopenta[d][1,3]dioxol-4-yl]-2,4-dichloropyrrolo[2,3-d]pyrimidine 196b (140 mg, 0.35 mmol, 1.0 eq) in 2M CH₃NH₂ in EtOH (2 mL). The resulting mixture was stirred for 2 h at 80° C., then concentrated to afford in 7-[(3aS,4R,6R,6aR)-2,2-dimethyl-6-phenyl-tetrahydro-3aH-cyclopenta[d][1,3]dioxol-4-yl]-2-chloro-N-methylpyrrolo[2,3-d]pyrimidin-4-amine 197b (100 mg, 72%) as a yellow solid. LCMS (conditions: L-column3 ODS, 50*3.0 mm, 3.0 μm, Mobile Phase A: Water (0.05% aqueous ammonia), Mobile Phase B: Acetonitrile, 5%-100% B-2 min, 1.50 mL/min): T_{R}=1.31 min; ES m/z [M+H]⁺: 399, 401.

### (1R,2S,3R,5R)-3-(2-Chloro-4-(methylamino)-7H-pyrrolo[2,3-d]pyrimidin-7-yl)-5-phenylcyclopentane-1,2-diol (596)

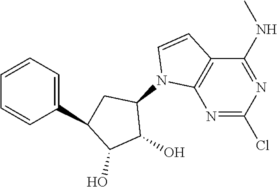 [SMILES: CNc1nc(Cl)nc2c1ccn2[C@@H]1C[C@H](c2ccccc2)[C@@H](O)[C@H]1O]

Into a 40 mL vial was added a solution of 7-[(3aS,4R,6R,6aR)-2,2-dimethyl-6-phenyl-tetrahydro-3aH-cyclopenta[d][1,3]dioxol-4-yl]-2-chloro-N-methylpyrrolo[2,3-d]pyrimidin-4-amine 197b (100 mq, 0.25 mmol, 1.0 eq) in 4M HCl in MeOH (2 mL). The resulting mixture was stirred for 15 min at 60° C., then concentrated. The residue was purified by reverse phase flash with the following conditions (Column: Welch Ultimate XB-C18, 50*250 mm, 10 μm; Mobile Phase A: 0.1% HCl, Mobile Phase B: ACETONITRILE; Flow rate: 90 mL/min; Gradient: 5% B-3 min, 10% B-45% B-10 min) to afford (1R,2S,3R,5R)-3-[2-chloro-4-(methylamino)pyrrolo[2,3-d]pyrimidin-7-yl]-5-phenylcyclopentane-1,2-diol HCl salt 596 (41.6 mg, 46%) as a white solid. LCMS (conditions: Cortecs C18+, 50*3.0 mm, 2.7 μm, Mobile Phase A: Water/0.02% TFA, Mobile Phase B: Acetonitrile, 5%-95% B-3 min, 1.50 mL/min): T_{R}=1.75 min; ES m/z [M+H]⁺: 359, 361. ¹H NMR (400 MHZ, DMSO-d₆+D₂O) δ 7.45-7.31 (m, 5H), 7.26-7.19 (m, 1H), 6.60 (d, J=3.5 Hz, 1H), 4.89-4.78 (m, 1H), 4.27 (t, J=6.7 Hz, 1H), 4.04 (t, J=6.5 Hz, 1H), 3.16-3.05 (m, 1H), 2.94 (s, 3H), 2.43-2.31 (m, 1H), 1.99 (q, J=11.6 Hz, 1H).

### Synthesis of (1R,2S,3R,5R)-3-(4-amino-5-(4-benzylthiazol-2-yl)-2-chloro-7H-pyrrolo[2,3-d]pyrimidin-7-yl)-5-((R)-1-methylpiperidin-3-yl)cyclopentane-1,2-diol (597R, structure assumed) and (1R,2S,3R,5R)-3-(4-amino-5-(4-benzylthiazol-2-yl)-2-chloro-7H-pyrrolo[2,3-d]pyrimidin-7-yl)-5-((S)-1-methylpiperidin-3-yl)cyclopentane-1,2-diol (597S, structure assumed)

### Tert-butyl(S)-3-((3aR,4R,6R,6aS)-6-(2,4-dichloro-5-iodo-7H-pyrrolo[2,3-d]pyrimidin-7-yl)-2,2-dimethyltetrahydro-4H-cyclopenta[d][1,3]dioxol-4-yl)piperidine-1-carboxylate (198b)

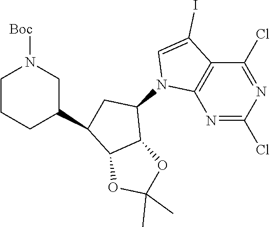 [SMILES: CC(C)(C)OC(=O)N1CCCC([C@H]2C[C@@H](n3cc(I)c4c(Cl)nc(Cl)nc43)[C@@H]3OC(C)(C)O[C@@H]32)C1]

To a stirred mixture of tert-butyl 3-[(3aR,4R,6S,6aS)-6-hydroxy-2,2-dimethyl-tetrahydro-3aH-cyclopenta[d][1,3]dioxol-4-yl]piperidine-1-carboxylate 114b (1.27 g, 3.73 mmol, 1.3 eq), PPh₃ (1.50 g, 5.73 mmol, 2.0 eq) and 2,4-dichloro-5-iodo-7H-pyrrolo[2,3-d]pyrimidine (0.90 g, 2.87 mmol, 1.0 eq) in toluene (7.0 mL) was added DBAD (1.32 g, 5.73 mmol, 2.0 eq) in toluene (3 mL) dropwise at 0° C. The resulting mixture was stirred for 5 h at 50° C. The resulting mixture was was quenched with water and extracted with ethyl acetate (3×30 mL). The combined organic phase was washed with brine, dried over anhydrous Na₂SO₄ and concentrated to give crude product which was further purified by column chromatography (1:2 tetrahydrofuran: petroleum ether) to afford tert-butyl 3-[(3aR,4R,6R,6aS)-6-{2,4-dichloro-5-iodopyrrolo[2,3-d]pyrimidin-7-yl}-2,2-dimethyl-tetrahydro-3aH-cyclopenta[d][1,3]dioxol-4-yl]piperidine-1-carboxylate 198b (1.0 g, 54%) as a colorless oil. LCMS (conditions: Cortecs C18+, 50*3.0 mm, 2.7 μm, Mobile Phase A: Water/0.05% TFA, Mobile Phase B: Acetonitrile, 2%-100% B-3 min), T_{R}=1.91 min; ES m/z [M+H]⁺: 637, 639.

### Tert-butyl(S)-3-((3aR,4R,6R,6aS)-6-(4-amino-2-chloro-5-iodo-7H-pyrrolo[2,3-d]pyrimidin-7-yl)-2,2-dimethyltetrahydro-4H-cyclopenta[d][1,3]dioxol-4-yl)piperidine-1-carboxylate (199b)

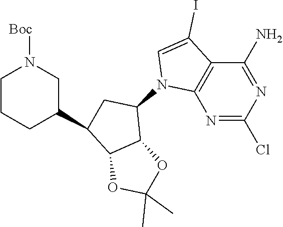 [SMILES: CC(C)(C)OC(=O)N1CCCC([C@H]2C[C@@H](n3cc(I)c4c(N)nc(Cl)nc43)[C@@H]3OC(C)(C)O[C@@H]32)C1]

Tert-butyl 3-[(3aR,4R,6R,6aS)-6-{2,4-dichloro-5-iodopyrrolo[2,3-d]pyrimidin-7-yl}-2,2-dimethyl-tetrahydro-3aH-cyclopenta[d][1,3]dioxol-4-yl]piperidine-1-carboxylate 198b (0.72 g, 1.13 mmol, 1.0 eq) was dissolved in 7M NH₃ in MeOH (10 mL). The reaction was stirred for 8 h at 80° C., then concentrated to give crude product which was further purified by column chromatography (1:1 tetrahydrofuran: petroleum ether) to afford tert-butyl 3-[(3aR,4R,6R,6aS)-6-{4-amino-2-chloro-5-iodopyrrolo[2,3-d]pyrimidin-7-yl}-2,2-dimethyl-tetrahydro-3aH-cyclopenta[d][1,3]dioxol-4-yl]piperidine-1-carboxylate 199b (0.50 g, 71%) as a white solid. LCMS (conditions: L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min), T_{R}=1.74 min; ES m/z [M+H]⁺: 618, 620. Tert-butyl(S)-3-((3aR,4R,6R,6aS)-6-(4-amino-5-(4-benzylthiazol-2-yl)-2-chloro-7H-pyrrolo[2,3-d]pyrimidin-7-yl)-2,2-dimethyltetrahydro-4H-cyclopenta[d][1,3]dioxol-4-yl)piperidine-1-carboxylate (200b)

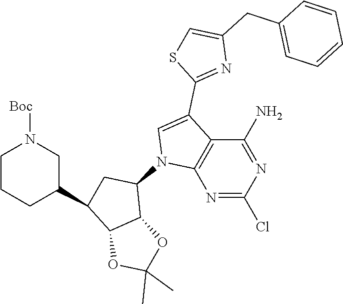 [SMILES: CC(C)(C)OC(=O)N1CCCC([C@H]2C[C@@H](n3cc(-c4nc(Cc5ccccc5)cs4)c4c(N)nc(Cl)nc43)[C@@H]3OC(C)(C)O[C@@H]32)C1]

To a stirred mixture of tert-butyl 3-[(3aR,4R,6R,6aS)-6-{4-amino-2-chloro-5-iodopyrrolo[2,3-d]pyrimidin-7-yl}-2,2-dimethyl-tetrahydro-3aH-cyclopenta[d][1,3]dioxol-4-yl]piperidine-1-carboxylate 199b (0.43 g, 0.70 mmol, 1.0 eq) and Pd (PPh₃) 2Cl₂ (49 mg, 0.07 mmol, 0.1 eq) in toluene (5 mL) were added (4-benzyl-1,3-thiazol-2-yl)(chloro) zinc (3.0 mL, 2.78 mmol, 4.0 eq) at 0° C. under N₂ atmosphere. The reaction was stirred for 12 h at 60° C., then quenched with saturated ammonium chloride solution at 0° C. The resulting mixture was extracted with ethyl acetate (3×30 mL). The combined organic layers were washed with brine and dried over anhydrous Na₂SO₄ and concentrated. The residue was purified by silica gel column chromatography (1:1 tetrahydrofuran: petroleum ether) to afford tert-butyl 3-[(3aR,4R,6R,6aS)-6-[4-amino-5-(4-benzyl-1,3-thiazol-2-yl)-2-chloropyrrolo[2,3-d]pyrimidin-7-yl]-2,2-dimethyl-tetrahydro-3aH-cyclopenta[d][1,3]dioxol-4-yl]piperidine-1-carboxylate 200b (0.33 g, 71%) as an off white solid. LCMS (L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-2 min), 1.50 mL/min): T_{R}=1.84 min; ES m/z [M+H]⁺: 665, 667.

### (1R,2S,3R,5R)-3-(4-Amino-5-(4-benzylthiazol-2-yl)-2-chloro-7H-pyrrolo[2,3-d]pyrimidin-7-yl)-5-((S)-piperidin-3-yl)cyclopentane-1,2-diol (201b)

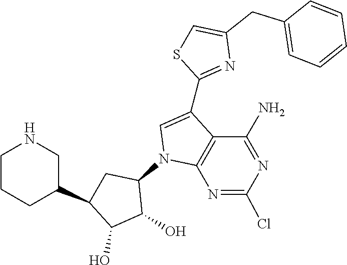 [SMILES: Nc1nc(Cl)nc2c1c(-c1nc(Cc3ccccc3)cs1)cn2[C@@H]1C[C@H](C2CCCNC2)[C@@H](O)[C@H]1O]

Tert-butyl 3-[(3aR,4R,6R,6aS)-6-[4-amino-5-(4-benzyl-1,3-thiazol-2-yl)-2-chloropyrrolo[2,3-d]pyrimidin-7-yl]-2,2-dimethyl-tetrahydro-3aH-cyclopenta[d][1,3]dioxol-4-yl]piperidine-1-carboxylate 200b (0.33 g, 0.50 mmol, 1.0 eq) was dissolved in 4M HCl in MeOH (3 mL). The reaction was stirred for 0.5 h at 60° C., then concentrated to afford (1R,2S,3R,5R)-3-[4-amino-5-(4-benzyl-1,3-thiazol-2-yl)-2-chloropyrrolo[2,3-d]pyrimidin-7-yl]-5-(piperidin-3-yl)cyclopentane-1,2-diol HCl salt 201b (0.20 g, 76%). LCMS (conditions: L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min), T_{R}=1.06 min; ES m/z [M+H]⁺: 525, 527.

### (1R,2S,3R,5R)-3-(4-Amino-5-(4-benzylthiazol-2-yl)-2-chloro-7H-pyrrolo[2,3-d]pyrimidin-7-yl)-5-((R)-1-methylpiperidin-3-yl)cyclopentane-1,2-diol (597R, structure assumed) and (1R,2S,3R,5R)-3-(4-amino-5-(4-benzylthiazol-2-yl)-2-chloro-7H-pyrrolo[2,3-d]pyrimidin-7-yl)-5-((S)-1-methylpiperidin-3-yl)cyclopentane-12-diol (5975 structure assumed)

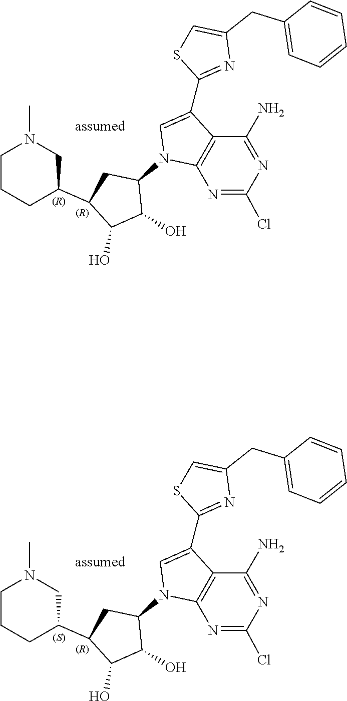

Into an 8 mL vial were added (1R,2S,3R,5R)-3-[4-amino-5-(4-benzyl-1,3-thiazol-2-yl)-2-chloropyrrolo[2,3-d]pyrimidin-7-yl]-5-(piperidin-3-yl)cyclopentane-1,2-diol HCl salt 201b (0.20 g, 0.38 mmol, 1.0 eq), DIEA (0.15 g, 1.14 mmol, 3.0 eq), NaBH₃CN (47.9 mg, 0.76 mmol, 2.0 eq), 30% HCHO in H₂O (92.7 mg, 1.14 mmol, 3.0 eq), MeOH (1.0 mL) and THF (1.0 mL). The resulting mixture was stirred for 2 h at room temperature. The reaction mixture was concentrated and the residue purified by Prep-HPLC with the following conditions (Column: Column: YMC-Actus Triart C18, 30*150 mm, 5 μm; Mobile Phase A: Water (10 mmol/L NH₄HCO₃+0.1% NH₃·H₂O), Mobile Phase B: ACETONITRILE; Flow rate: 30 ml/min; Gradient: 10% B to 60% B over 12 min) to afford the following diastereomers:

(1R,2S,3R,5R)-3-[4-Amino-5-(4-benzyl-1,3-thiazol-2-yl)-2-chloropyrrolo[2,3-d]pyrimidin-7-yl]-5-[(3R)-1-methylpiperidin-3-yl]cyclopentane-1,2-diol 597R (12.4 mg, 6%) as a white solid. LCMS (Cortecs C18+, 50*3.0 mm, 2.7 μm, Mobile Phase A: Water/0.02% TFA, Mobile Phase B: Acetonitrile, 5%-95% B-3 min), T_{R}=1.10 min; ES m/z [M+H]⁺: 539, 541. ¹H NMR (300 MHZ, DMSO-d₆) δ 9.43 (br, s, 1H), 8.17 (s, 1H), 7.84 (br, s, 1H), 7.40-7.17 (m, 6H), 4.85 (br, s, 1H), 4.77-4.68 (m, 1H), 4.59 (br, s, 1H), 4.20-4.14 (m, 1H), 4.07 (s, 2H), 3.80-3.71 (m, 1H), 2.97 (d, J=9.8 Hz, 1H), 2.72-2.66 (m, 1H), 2.18 (s, 3H), 2.06-1.94 (m, 1H), 1.85-1.72 (m, 1H), 1.72-1.32 (m, 7H), 0.93-0.79 (m, 1H).

(1R,2S,3R,5R)-3-[4-Amino-5-(4-benzyl-1,3-thiazol-2-yl)-2-chloropyrrolo[2,3-d]pyrimidin-7-yl]-5-[(3S)-1-methylpiperidin-3-yl]cyclopentane-1,2-diol 597S (27.4 mg, 13%) as a white solid. LCMS (Cortecs C18+, 50*3.0 mm, 2.7 μm, Mobile Phase A: Water/0.02% TFA, Mobile Phase B: Acetonitrile, 5%-95% B-3 min), T_{R}=1.06 min; ES m/z [M+H]⁺: 539, 541. ¹H NMR (400 MHZ, DMSO-d₆) δ 9.42 (br, s, 1H), 8.19 (s, 1H), 7.83 (br, s, 1H), 7.38-7.26 (m, 4H), 7.26-7.19 (m, 2H), 4.82 (d, J=6.7 Hz, 1H), 4.80-4.69 (m, 1H), 4.60 (br, s, 1H), 4.20-4.11 (m, 1H), 4.07 (s, 2H), 3.81-3.74 (m, 1H), 2.67-2.62 (m, 2H), 2.13 (s, 3H), 2.06-1.96 (m, 1H), 1.95-1.78 (m, 2H), 1.69-1.48 (m, 5H), 1.48-1.38 (m, 1H), 0.96 (q, J=11.5 Hz, 1H).

### Synthesis of (1R,2S,3R,5S)-3-(4-(methylamino)-7H-pyrrolo[2,3-d]pyrimidin-7-yl)-5-((3-((phenethylamino)methyl)cyclobutyl)methyl)cyclopentane-1,2-diol (598)

### Methyl 3-(((3aR,4R,6aR)-2,2-dimethyl-6-oxotetrahydro-4H-cyclopenta[d][1,3]dioxol-4-yl)methylene)cyclobutane-1-carboxylate (20c)

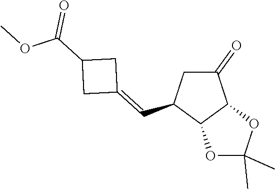

Into a 20 mL sealed tube were added (3aR,6aR)-2,2-dimethyl-3aH,6aH-cyclopenta[d][1,3]dioxol-4-one (800 mg, 5.19 mmol, 1.0 eq), methyl 3-[(4,4,5,5-tetramethyl-1,3,2-dioxaborolan-2-yl)methylidene]cyclobutane-1-carboxylate (1.96 g, 7.78 mmol, 1.5 eq) [Eur. J. Org. Chem., 2019, 2019 (33), 5624-5635], [Rh(COD)Cl]2 (511 mg, 1.04 mmol, 0.2 eq), KOH (87 mg, 1.56 mmol, 0.3 eq), H₂O (1.0 mL) and CH₃OH (10.0 mL) at room temperature. The final reaction mixture was irradiated with microwave radiation for 2 h at 40° C. The reaction was quenched with water (30 mL) and then extracted with EtOAc (3×30 mL). The organic layer was dried over anhydrous Na₂SO₄, filtered and the filtrate was concentrated under reduced pressure. The residue was purified by silica gel column chromatography, eluted with (95:5 AcOEt: petroleum ether) to afford methyl 3-{[(3aR,4R,6aR)-2,2-dimethyl-6-oxo-tetrahydrocyclopenta[d][1,3]dioxol-4-yl]methylidene}cyclobutane-1-carboxylate 20 (750 mg, 51%) as an off-white solid. LCMS [XBridge C18, 50*3.0 mm, 3.5 μm; Mobile Phase A: Water/5 mM NH₄HCO₃, Mobile Phase B: Methanol, 10%-95% B-3 min, 1.00 mL/min, ES, m/z]: T_{R}=1.82 min, ES m/z [M+H]⁺: 281.

### Methyl 3-(((3aR,4S,6aR)-2,2-dimethyl-6-oxotetrahydro-4H-cyclopenta[d][1,3]dioxol-4-yl)methyl)cyclobutane-1-carboxylate (21c)

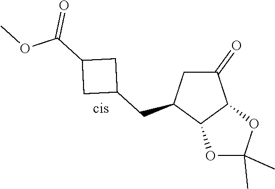

To a solution of methyl 3-{[(3aR,4R,6aR)-2,2-dimethyl-6-oxo-tetrahydrocyclopenta[d][1,3]dioxol-4-yl]methylidene}cyclobutane-1-carboxylate 20c (720 mg, 2.57 mmol, 1.0 eq) in MeOH (10 mL) was added 10% Pd/C (109 mg) in a 40 mL vial. The mixture was hydrogenated at room temperature for 1 h under a hydrogen balloon, filtered through a Celite pad and concentrated under reduced pressure to afford methyl 3-{[(3aR,4S,6aR)-2,2-dimethyl-6-oxo-tetrahydrocyclopenta[d][1,3]dioxol-4-yl]methyl}cyclobutane-1-carboxylate 21c (620 mg, crude) as an off-white solid. LCMS [XBridge C18, 50*3.0 mm, 3.5 μm; Mobile Phase A: Water/5 mM NH₄HCO₃, Mobile Phase B: Methanol, 10%-95% B-3 min, 1.00 mL/min, ES, m/z]: T_{R}=1.91 min, ES m/z [M+H]⁺: 383.

### Methyl 3-(((3aR,4S,6S,6aS)-6-hydroxy-2,2-dimethyltetrahydro-4H-cyclopenta[d][1,3]dioxol-4-yl)methyl)cyclobutane-1-carboxylate (22c)

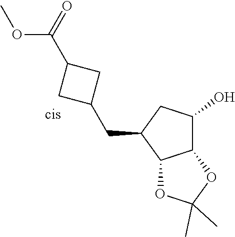 [SMILES: COC(=O)C1CC(C[C@H]2C[C@H](O)[C@@H]3OC(C)(C)O[C@H]23)C1]

To a stirred solution of methyl 3-{[(3aR,45,6aR)-2,2-dimethyl-6-oxo-tetrahydrocyclopenta[d][1,3]dioxol-4-yl]methyl}cyclobutane-1-carboxylate 21c (620 mg, 2.20 mmol, 1.0 eq) in MeOH (8.0 mL) were added NaBH₄ (124 mg, 3.30 mmol, 1.5 eq) in portions at 0° C. under. The resulting mixture was stirred for 1 h at room temperature. The reaction was quenched with water (30 mL) and then extracted with EtOAc (3×30 mL). The organic layer was dried over anhydrous Na₂SO₄, filtered and the filtrate was concentrated under reduced pressure. The crude product was purified by Prep-HPLC [Column: Xtimate C18 50×250 mm, 10 μm; Mobile Phase A: 0.5% NH₃·H₂O, Mobile Phase B: ACN; Flow rate: 90 mL/min; Gradient: 90% 15 min; Detector, UV 220 nm]to afford methyl 3-{[(3aR,4S,6S,6aS)-6-hydroxy-2,2-dimethyl-tetrahydro-3aH-cyclopenta[d][1,3]dioxol-4-yl]methyl}cyclobutane-1-carboxylate 22c (500 mg, 80%) as an off-white solid. LCMS [Xbridge Shield C18, 50*3.0 mm, 3.5 μm; Mobile Phase A: Water/0.05% Ammonia, Mobile Phase B: Acetonitrile, 5%-100% B-3 min, 1.20 mL/min, ES, m/z]: T_{R}=1.27 min, ES m/z [M+H]⁺: 285.

### 3-(((3aR,4S,6S,6aS)-6-Hydroxy-2,2-dimethyltetrahydro-4H-cyclopenta[d][1,3]dioxol-4-yl)methyl)-N-phenethylcyclobutane-1-carboxamide (73c)

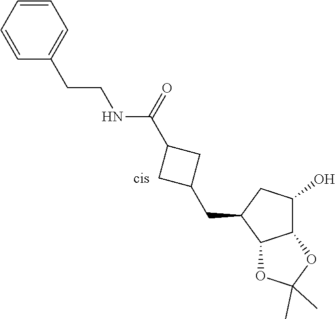 [SMILES: CC1(C)O[C@@H]2[C@@H](CC3CC(C(=O)NCCc4ccccc4)C3)C[C@H](O)[C@@H]2O1]

Into a 40 mL vial were added methyl 3-{[(3aR,4S,6S,6aS)-6-hydroxy-2,2-dimethyl-tetrahydro-3aH-cyclopenta[d][1,3]dioxol-4-yl]methyl}cyclobutane-1-carboxylate 22c (500 mg, 1.76 mmol, 1.0 eq), 2-phenylethylamine (255 mg, 2.11 mmol, 1.2 eq) and toluene (10.0 mL) at room temperature. 1M AlMe₃ in toluene (3.51 mL, 3.51 mmol, 2.0 eq) was added dropwise at 0° C. The resulting mixture was stirred for 2 h at 70° C. The reaction was quenched with water (30 mL) and then extracted with EtOAc (3×30 mL). The organic layer was dried over anhydrous Na₂SO₄, filtered and the filtrate was concentrated under reduced pressure. The residue was purified by silica gel column chromatography (1:1 AcOEt: petroleum ether) to afford 3-{[(3aR,4S,6S,6aS)-6-hydroxy-2,2-dimethyl-tetrahydro-3aH-cyclopenta[d][1,3]dioxol-4-yl]methyl}-N-(2-phenylethyl)cyclobutane-1-carboxamide 23c (520 mg, 79%) as a white solid. LCMS [Xbridge Shield C18, 50*3.0 mm, 3.0 μm; Mobile Phase A: Water/0.05% Ammonia, Mobile Phase B: Acetonitrile, 5%-100% B-3 min, 1.50 mL/min, ES, m/z]: T_{R}=1.34 min, ES m/z [M+H]⁺: 374.

### tert-Butyl ((3-(((3aR,45,6S,6aS)-6-hydroxy-2,2-dimethyltetrahydro-4H-cyclopenta[d][1,3]dioxol-4-yl)methyl)cyclobutyl)methyl)(phenethyl)carbamate (24c)

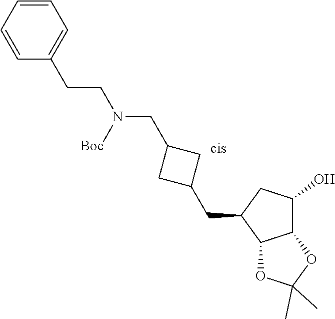 [SMILES: CC(C)(C)OC(=O)N(CCc1ccccc1)CC1CC(C[C@H]2C[C@H](O)[C@@H]3OC(C)(C)O[C@H]23)C1]

To a stirred solution of 3-{[(3aR,4S,6S,6aS)-6-hydroxy-2,2-dimethyl-tetrahydro-3aH-cyclopenta[d][1,3]dioxol-4-yl]methyl}-N-(2-phenylethyl)cyclobutane-1-carboxamide 23c (520 mg, 1.39 mmol, 1.0 eq) in THF (5.0 mL) was added 1M DIBAI-H in THF (2.78 mL, 2.78 mmol, 2.0 eq) dropwise at 0° C. The resulting mixture was stirred for 1 h at room temperature. The reaction was quenched with 1.0 mL H₂O at 0° C. and concentrated under reduced pressure. To the residue was added Boc₂O (607 mg, 2.78 mmol, 2.0 eq), DCM (5.0 mL) and TEA (422 mg, 4.18 mmol, 3.0 eq). The resulting mixture was stirred for additional 1h at room temperature. The reaction was quenched with water (30 mL) and then extracted with EtOAc (3×30 mL). The organic layer was dried over anhydrous Na₂SO₄, filtered and the filtrate was concentrated under reduced pressure. The residue was purified by silica gel column chromatography (1:1 AcOEt: petroleum ether) to afford tert-butyl N-[(3-{[(3aR,45,6S,6aS)-6-hydroxy-2,2-dimethyl-tetrahydro-3aH-cyclopenta[d][1,3]dioxol-4-yl]methyl}cyclobutyl)methyl]-N-(2-phenylethyl)carbamate 24c (270 mg, 42%) as a yellow solid. LCMS [XBridge C18, 50*3.0 mm, 3.5 μm; Mobile Phase A: Water/5 mM NH₄HCO₃, Mobile Phase B: Methanol, 10%-95% B-3 min, 1.00 mL/min, ES, m/z]: T_{R}=2.42 min, ES m/z [M+H]⁺: 460.

### (3aR,4S,6S,6aR)-6-((3-(((tert-Butoxycarbonyl)(phenethyl)amino)methyl)cyclobutyl)methyl)-2,2-dimethyltetrahydro-4H-cyclopenta[d][1,3]dioxol-4-yl trifluoromethanesulfonate (25c)

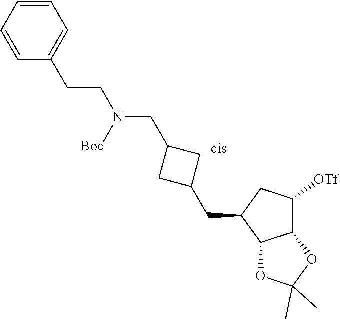 [SMILES: CC(C)(C)OC(=O)N(CCc1ccccc1)CC1CC(C[C@H]2C[C@H](OS(=O)(=O)C(F)(F)F)[C@@H]3OC(C)(C)O[C@H]23)C1]

Into a 40 mL vial were added tert-butyl N-[(3-{[(3aR,4S,6S,6aS)-6-hydroxy-2,2-dimethyl-tetrahydro-3aH-cyclopenta[d][1,3]dioxol-4-yl]methyl}cyclobutyl)methyl]-N-(2-phenylethyl)carbamate 24c (100 mg, 0.22 mmol, 1.0 eq), DCM (2.0 mL) and pyridine (43 mg, 0.55 mmol, 2.5 eq) at room temperature. Tf₂O (122 mg, 0.44 mmol, 2.0 eq) was added dropwise at 0° C. The resulting mixture was stirred for 1 h at room temperature, then concentrated under reduced pressure to afford tert-butyl N-[(3-{[(3aR,4S,6S,6aR)-2,2-dimethyl-6-(trifluoromethanesulfonyloxy)-tetrahydro-3aH-cyclopenta[d][1,3]dioxol-4-yl]methyl}cyclobutyl)methyl]-N-(2-phenylethyl)carbamate 25c (100 mg, 77%) as a yellow oil. This was used in the next step directly without further purification. LCMS [L-column3 ODS, 50*3.0 mm, 3.0 μm; Mobile Phase A: Water/5 mM NH₄HCO₃+0.05% Ammonia, Mobile Phase B: Acetonitrile/5% water, 5%-95% B-5 min, 1.00 mL/min, ES, m/z]: T_{R}=3.67 min, ES m/z [M+H]⁺: 592.

### tert-Butyl ((3-(((3aR,4S,6R,6aS)-6-(4-((4-methoxybenzyl)(methyl)amino)-7H-pyrrolo[2,3-d]pyrimidin-7-yl)-2,2-dimethyltetrahydro-4H-cyclopenta[d][1,3]dioxol-4-yl)methyl)cyclobutyl)methyl)(phenethyl)carbamate (26c)

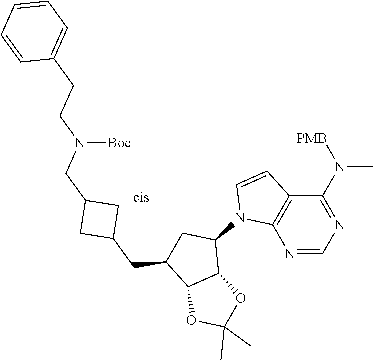

To a stirred solution of tert-butyl N-[(3-{[(3aR,45,65,6aR)-2,2-dimethyl-6-(trifluoromethanesulfonyloxy)-tetrahydro-3aH-cyclopenta[d][1,3]dioxol-4-yl]methyl}cyclobutyl)methyl]-N-(2-phenylethyl)carbamate 25c (100 mg, 0.17 mmol, 1.0 eq) and N-[(4-methoxyphenyl)methyl]-N-methyl-7H-pyrrolo[2,3-d]pyrimidin-4-amine (54 mg, 0.20 mmol, 1.2 eq) [WO2020055672]in DMF (2.0 mL) was added NaH (16 mg, 0.68 mmol, 4.0 eq) in portions at 0° C. The resulting mixture was stirred for 1 h at room temperature. The reaction was quenched with water (30 mL) and then extracted with EtOAc (3×30 mL). The organic layer was dried over anhydrous Na₂SO₄, filtered and the filtrate was concentrated under reduced pressure. The residue was purified by silica gel column chromatography (1:1 AcOEt: petroleum ether) to afford tert-butyl N-[(3-{[(3aR,4S,6R,6aS)-6-(4-{[(4-methoxyphenyl)methyl](methyl)amino}pyrrolo[2,3-d]pyrimidin-7-yl)-2,2-dimethyl-tetrahydro-3aH-cyclopenta[d][1,3]dioxol-4-yl]methyl}cyclobutyl)methyl]-N-(2-phenylethyl)carbamate 26c (32 mg, 26%) as a yellow solid. LCMS [XBridge C18, 50*3.0 mm, 3.5 μm; Mobile Phase A: Water/5 mM NH₄HCO₃, Mobile Phase B: Methanol, 10%-95% B-3 min, 1.50 mL/min, ES, m/z]: T_{R}=2.64 min, ES m/z [M+H]⁺: 710.

### (1R,2S,3R,5S)-3-(4-(methylamino)-7H-pyrrolo[2,3-d]pyrimidin-7-yl)-5-((3-((phenethylamino)methyl)cyclobutyl)methyl)cyclopentane-1,2-diol (598)

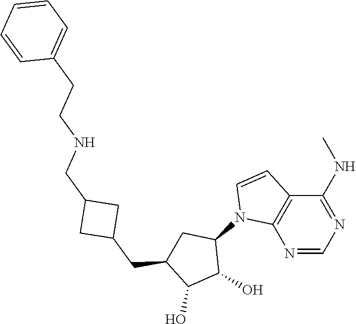 [SMILES: CNc1ncnc2c1ccn2[C@@H]1C[C@H](CC2CC(CNCCc3ccccc3)C2)[C@@H](O)[C@H]1O]

Into a 8 mL vial were added tert-butyl N-[(3-{[(3aR,45,6R,6aS)-6-(4-{[(4-methoxyphenyl)methyl](methyl)amino}pyrrolo[2,3-d]pyrimidin-7-yl)-2,2-dimethyl-tetrahydro-3aH-cyclopenta[d][1,3]dioxol-4-yl]methyl}cyclobutyl)methyl]-N-(2-phenylethyl)carbamate 26c (32 mg, 0.04 mmol, 1.0 eq) and TFA (2.0 mL) at room temperature. The resulting mixture was stirred for 1 h at 50° C. then concentrated under reduced pressure. The mixture was basified to pH>8 with NH₃. H₂O and purified by Prep-HPLC [Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 20% B to 50% B in 8 min; Detector, UV 220&254 nm]to afford (1R,2S,3R,5S)-3-[4-(methylamino)pyrrolo[2,3-d]pyrimidin-7-yl]-5-[(3-{[(2-phenylethyl)amino]methyl}cyclobutyl)methyl]cyclopentane-1,2-diol 598 (5.9 mg, 29%) as an off-white solid. ¹H NMR (400 MHZ, DMSO-d₆) δ 8.11 (s, 1H), 7.40-7.32 (m, 1H), 7.30-7.22 (m, 2H), 7.24-7.11 (m, 4H), 6.51 (d, J=3.5 Hz, 1H), 4.81-4.70 (m, 2H), 4.54 (d, J=4.9 Hz, 1H), 4.18-4.08 (m, 1H), 3.67-3.59 (m, 1H), 2.94 (d, J=4.7 Hz, 3H), 2.75-2.64 (m, 4H), 2.62-2.56 (m, 1H), 2.50-2.46 (m, 1H), 2.23-2.04 (m, 5H), 1.83-1.56 (m, 3H), 1.51-1.32 (m, 2H), 1.26-1.18 (m, 2H). LCMS [ZORBAX SB-Aq, 50*4.6 mm, 1.8 μm; Mobile Phase A: Water/0.02% TFA, Mobile Phase B: Acetonitrile, 5%-95% B-3 min, 1.50 mL/min, ES, m/z]: T_{R}=1.15 min, ES m/z [M+H]⁺: 450.

### Synthesis of (1R,2S,3R,5R)-3-[4-Amino-5-(1,3-thiazol-2-yl)pyrrolo[2,3-d]pyrimidin-7-yl]-5-{[(3-{[2-(4-fluorophenyl)ethyl]amino}propyl)(1H-pyrazol-4-yl)amino]methyl}cyclopentane-1,2-diol (599)

### 7-[(3aS,4R,6R,6aR)-6-{[(tert-Butyldimethylsilyl)oxy]methyl}-2,2-dimethyl-tetrahydro-3aH-cyclopenta[d][1,3]dioxol-4-yl]-4-chloropyrrolo[2,3-d]pyrimidine (180c)

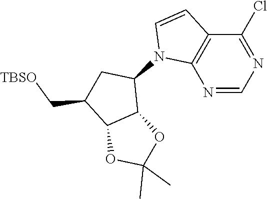 [SMILES: CC1(C)O[C@@H]2[C@@H](CO[Si](C)(C)C(C)(C)C)C[C@@H](n3ccc4c(Cl)ncnc43)[C@@H]2O1]

To a solution of [(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]methanol 5 (0.50 g, 1.54 mmol, 1.0 eq) in DCM (10.0 mL) was added 1H-imidazole (0.31 g, 4.60 mmol, 3.0 eq) and DMAP (0.02 g, 0.10 mmol, 0.1 eq). To the resulting mixture was added TBSCl (0.28 g, 1.80 mmol, 1.2 equiv), portionwise over 2 min at 0° C., and the resulting mixture was stirred for 1 h at room temperature. The reaction mixture was quenched with saturated aq. NaHCO₃ at 0° C. and extracted with AcOEt (3×10 mL). The combined organic layers were washed with brine and dried over anhydrous Na₂SO₄. After filtration, the filtrate was concentrated under reduced pressure and the crude residue was purified by silica gel column chromatography (75:25 Petroleum ether: AcOEt) to afford 7-[(3aS,4R,6R,6aR)-6-{[(tert-butyldimethylsilyl)oxy]methyl}-2,2-dimethyl-tetrahydro-3aH-cyclopenta[d][1,3]dioxol-4-yl]-4-chloropyrrolo[2,3-d]pyrimidine 180c (560 mg, 83%) as a light yellow oil. LCMS [conditions L-column3 ODS, 50*3.0 mm, 3.0 μm, Mobile Phase A: Water/5 mM NH₄HCO₃+0.05% Ammonia, Mobile Phase B: Acetonitrile/5% water, 10%-95% B-3 min (+), 1.50 L/min, ES, m/z,]: T_{R}=2.58 min; [M+H]⁺: 438. ¹H NMR (300 MHZ, DMSO-d₆) δ 8.63 (s, 1H), 7.92 (d, J=3.7 Hz, 1H), 6.72 (d, J=3.6 Hz, 1H), 5.15-5.02 (m, 1H), 4.95 (t, J=6.6 Hz, 1H), 4.59-4.46 (m, 1H), 3.80-3.59 (m, 2H), 2.37-2.06 (m, 3H), 1.49 (s, 3H), 1.22 (s, 3H), 0.88 (s, 9H), 0.06 (s, 6H).

### [(3aR,4R,6R,6aS)-6-{5-Bromo-4-chloropyrrolo[2,3-d]pyrimidin-7-yl}-2,2-dimethyl-tetrahydro-3aH-cyclopenta[d][1,3]dioxol-4-yl]methanol (153c)

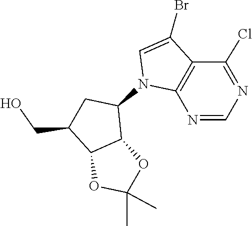 [SMILES: CC1(C)O[C@@H]2[C@@H](CO)C[C@@H](n3cc(Br)c4c(Cl)ncnc43)[C@@H]2O1]

To a solution of 7-[(3aS,4R,6R,6aR)-6-{[(tert-butyldimethylsilyl)oxy]methyl}-2,2-dimethyl-tetrahydro-3aH-cyclopenta[d][1,3]dioxol-4-yl]-4-chloropyrrolo[2,3-d]pyrimidine 180c (0.20 g, 0.40 mmol, 1.0 eq) in DMF (2.0 mL), at 0° C., was added a solution of NBS (0.10 g, 0.50 mmol, 1.2 eq) in DMF (2.0 mL), dropwise over 2 min and the resulting mixture was stirred for additional 8 h at room temperature. The reaction mixture was quenched with saturated aq. Na₂S203 at 0° C. and extracted with AcOEt (3×10 mL). The combined organic layers were washed with saturated aq. NaHCO₃, brine, and then dried over anhydrous Na₂SO₄. After filtration, the filtrate was concentrated under reduced pressure and the crude residue was purified by Prep-HPLC (Column: Spherical C18, 20-40 μm, 120g; Mobile Phase A: Water, Mobile Phase B: ACN; Flow rate: 80 mL/min; Gradient: 30% B to 80% B in 10 min, 78% B; Wave Length: 254 nm) to afford [(3aR,4R,6R,6aS)-6-{5-bromo-4-chloropyrrolo[2,3-d]pyrimidin-7-yl}-2,2-dimethyl-tetrahydro-3aH-cyclopenta[d][1,3]dioxol-4-yl]methanol 153c (110 mg, 60%) as a pale brown oil. LCMS [conditions XBridge C18, 50*3.0 mm, 3.5 μm, Mobile Phase A: Water/5 mM NH₄HCO₃, Mobile Phase B: Methanol, 10%-95% B-3 min (+), 1.50 L/min, ES, m/z]: T_{R}=2.04; [M+H]⁺: 402, 404.1H NMR (300 MHZ, DMSO-d₆) δ 8.69 (s, 1H), 8.26 (s, 1H), 5.21-5.04 (m, 1H), 4.95-4.72 (m, 2H), 4.59-4.45 (m, 1H), 3.59-3.45 (m, 2H), 2.34-2.18 (m, 2H), 1.48 (s, 3H), 1.23 (s, 3H).

### (3aR,4S,6R,6aS)-6-{5-Bromo-4-chloropyrrolo[2,3-d]pyrimidin-7-yl}-2,2-dimethyl-tetrahydro-3aH-cyclopenta[d][1,3]dioxole-4-carbaldehyde (154c)

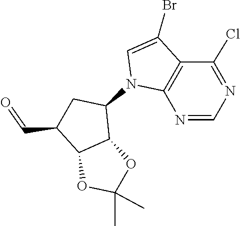 [SMILES: CC1(C)O[C@@H]2[C@H](O1)[C@@H](C=O)C[C@H]2n1cc(Br)c2c(Cl)ncnc21]

To a solution of [(3aR,4R,6R,6aS)-6-{5-bromo-4-chloropyrrolo[2,3-d]pyrimidin-7-yl}-2,2-dimethyl-tetrahydro-3aH-cyclopenta[d][1,3]dioxol-4-yl]methanol 153c (0.11 g, 0.30 mmol, 1.0 eq) in DCM (1.0 mL), at 0° C., was added DMP (0.15 g, 0.3 mmol, 1.3 eq) and the reaction mixture was stirred for 1 h at room temperature. The resulting mixture was concentrated under vacuum and the crude residue was purified by silica gel column chromatography (60:40 Petroleum ether: AcOEt) to afford (3aR,4S,6R,6aS)-6-{5-bromo-4-chloropyrrolo[2,3-d]pyrimidin-7-yl}-2,2-dimethyl-tetrahydro-3aH-cyclopenta[d][1,3]dioxole-4-carbaldehyde 154c (100 mg, 91%) as a colorless oil. LCMS [conditions Proshell HPH-C18, 50*3.0 mm, 2.7 μm, Mobile Phase A: Water (0.05% ammonia water, Mobile Phase B: Acetonitrile, 30%-100% B]: T_{R}=5.14 min; [M+H]⁺: 400, 402.1H NMR (300 MHZ, DMSO-d₆) δ 9.74 (s, 1H), 8.69 (s, 1H), 8.20 (s, 1H), 5.26-5.15 (m, 1H), 5.14-5.03 (m, 1H), 4.99-4.89 (m, 1H), 3.21-3.08 (m, 1H), 2.47-2.18 (m, 2H), 1.49 (s, 3H), 1.27 (s, 3H).

### tert-Butyl-N-[3-({[(3aR,4R,6R,6aS)-6-{5-Bromo-4-chloropyrrolo[2,3-d]pyrimidin-7-yl}-2,2-dimethyl-tetrahydro-3aH-cyclopenta[d][1,3]dioxol-4-yl]methyl}(1H-pyrazol-4-yl)amino)propyl]-N-[2-(4-fluorophenyl)ethyl]carbamate (183c)

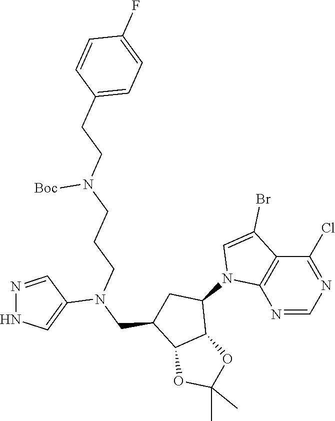 [SMILES: CC(C)(C)OC(=O)N(CCCN(C[C@H]1C[C@@H](n2cc(Br)c3c(Cl)ncnc32)[C@@H]2OC(C)(C)O[C@H]12)c1cn[nH]c1)CCc1ccc(F)cc1]

To a solution of tert-butyl-N-[2-(4-fluorophenyl)ethyl]-N-[3-(1H-pyrazol-4-ylamino)propyl]carbamate 189c (0.14 g, 0.40 mmol, 1.5 eq) in DCM (1.0 mL) was added HOAc (0.15 g, 2.50 mmol, 10 eq) and NaBH (OAc) 3 (0.10 g, 0.50 mmol, 2.0 eq) and the resulting mixture was stirred for 10 min at 0° C. To the above mixture was added (3aR,4S,6R,6aS)-6-{5-bromo-4-chloropyrrolo[2,3-d]pyrimidin-7-yl}-2,2-dimethyl-tetrahydro-3aH-cyclopenta[d][1,3]dioxole-4-carbaldehyde 182c (0.10 g, 0.20 mmol, 1.0 eq) in DCM (1.0 mL), dropwise over 3 min, at 0° C. and the reaction mixture was stirred at 0° C. for 30 min. The resulting mixture was concentrated under reduced pressure and neutralized to pH 7 with TEA and the crude residue was purified by silica gel column chromatography (96:4 DCM: MeOH) to afford tert-butyl-N-[3-({[(3aR,4R,6R,6aS)-6-{5-bromo-4-chloropyrrolo[2,3-d]pyrimidin-7-yl}-2,2-dimethyl-tetrahydro-3aH-cyclopenta[d][1,3]dioxol-4-yl]methyl}(1H-pyrazol-4-yl)amino)propyl]-N-[2-(4-fluorophenyl)ethyl]carbamate 183c (160 mg, 86%) as a colorless oil. LCMS [conditions XBridge C18, 50*3.0 mm, 3.5 μm, Mobile Phase A: Water/5 mM NH₄HCO₃, Mobile Phase B: Methanol, 10%-95% B-5 min (+), 1.50 L/min, ES, m/z)]: T_{R}=4.23 min; [M+H]⁺: 746, 748.1H NMR (300 MHZ, DMSO-d₆) δ 12.11 (s, 1H), 8.68 (s, 1H), 8.25 (s, 1H), 7.31-6.96 (m, 6H), 5.22-5.02 (m, 1H), 4.97-4.78 (m, 1H), 4.60-4.38 (m, 1H), 3.22-3.06 (m, 4H), 3.04-2.84 (m, 3H), 2.82-2.64 (m, 2H), 2.40-2.32 (m, 4H), 1.91 (s, 3H), 1.68-1.55 (m, 2H), 1.45 (s, 3H), 1.32 (s, 9H).

### tert-Butyl-N-[3-({[(3aR,4R,6R,6aS)-6-{4-amino-5-bromopyrrolo[2,3-d]pyrimidin-7-yl}-2,2-dimethyl-tetrahydro-3aH-cyclopenta[d][1,3]dioxol-4-yl]methyl}(1H-pyrazol-4-yl)amino)propyl]-N-[2-(4-fluorophenyl)ethyl]carbamate (184c)

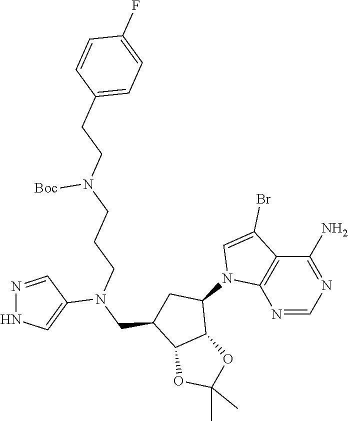 [SMILES: CC(C)(C)OC(=O)N(CCCN(C[C@H]1C[C@@H](n2cc(Br)c3c(N)ncnc32)[C@@H]2OC(C)(C)O[C@H]12)c1cn[nH]c1)CCc1ccc(F)cc1]

To tert-butyl-N-[3-({[(3aR,4R,6R,6aS)-6-{5-bromo-4-chloropyrrolo[2,3-d]pyrimidin-7-yl}-2,2-dimethyl-tetrahydro-3aH-cyclopenta[d][1,3]dioxol-4-yl]methyl}(1H-pyrazol-4-yl)amino)propyl]-N-[2-(4-fluorophenyl)ethyl]carbamate 183c (0.12 g, 0.20 mmol, 1.0 eq) was added 7M NH₃ in MeOH (2.0 mL) and the reaction mixture was stirred at 80° C. for 16 h. The resulting mixture was concentrated under vacuum to afford tert-butyl-N-[3-({[(3aR,4R,6R,6aS)-6-{4-amino-5-bromopyrrolo[2,3-d]pyrimidin-7-yl}-2,2-dimethyl-tetrahydro-3aH-cyclopenta[d][1,3]dioxol-4-yl]methyl}(1H-pyrazol-4-yl)amino)propyl]-N-[2-(4-fluorophenyl)ethyl]carbamate 184c (150 mg, crude). LCMS [conditions Proshell HPH-C18, 50*3.0 mm, 2.7 μm, Mobile Phase A: Water (0.05% ammonia water), Mobile Phase B: Acetonitrile, 5%-100% B-2 min (+), 1.50 L/min, ES, m/z]: T_{R}=1.32 min; [M+H]⁺: 727, 729.

### tert-Butyl-NV-[3-({[(3aR,4R,6R,6aS)-6-[4-amino-5-(1,3-thiazol-2-yl)pyrrolo[2,3-d]pyrimidin-7-yl]-2,2-dimethyl-tetrahydro-3aH-cyclopenta[d][1,3]dioxol-4-yl]methyl}(1H-pyrazol-4-yl)amino)propyl]-N-[2-(4-fluorophenyl)ethyl]carbamate (185c)

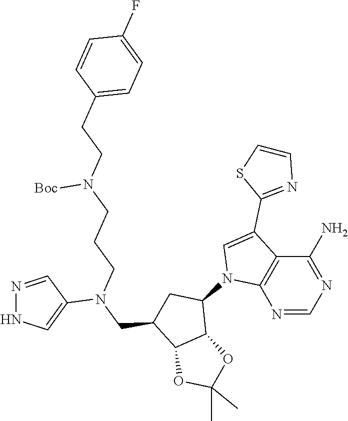 [SMILES: CC(C)(C)OC(=O)N(CCCN(C[C@H]1C[C@@H](n2cc(-c3nccs3)c3c(N)ncnc32)[C@@H]2OC(C)(C)O[C@H]12)c1cn[nH]c1)CCc1ccc(F)cc1]

To a solution of tert-butyl-N-[3-({[(3aR,4R,6R,6aS)-6-{4-amino-5-bromopyrrolo[2,3-d]pyrimidin-7-yl}-2,2-dimethyl-tetrahydro-3aH-cyclopenta[d][1,3]dioxol-4-yl]methyl}(1H-pyrazol-4-yl)amino)propyl]-N-[2-(4-fluorophenyl)ethyl]carbamate 184c (0.15 g, 0.20 mmol, 1.0 eq) in toluene (2.0 mL) was added 2-(tributylstannyl)-1,3-thiazole (0.23 g, 0.60 mmol, 3.0 eq) and Pd (PPh₃) 2Cl₂ (14.5 mg, 0.02 mmol, 0.1 eq), at room temperature under N₂, and the reaction mixture was stirred at 120° C. for 2 h. The resulting mixture was concentrated under vacuum and the crude residue was purified by Prep-TLC (12:1 DCM: MeOH) to afford tert-butyl-N-[3-({[(3aR,4R,6R,6aS)-6-[4-amino-5-(1,3-thiazol-2-yl)pyrrolo[2,3-d]pyrimidin-7-yl]-2,2-dimethyl-tetrahydro-3aH-cyclopenta[d][1,3]dioxol-4-yl]methyl}(1H-pyrazol-4-yl)amino)propyl]-N-[2-(4-fluorophenyl)ethyl]carbamate 185c (30 mg, 20%) as an off-white solid. LCMS [conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min (+), 1.50 L/min, ES, m/z]: T_{R}=2.16 min; [M+H]⁺: 732. ¹H NMR (300 MHZ, DMSO-d₆) δ 8.23 (s, 1H), 8.11 (s, 1H), 7.83 (d, J=3.4 Hz, 1H), 7.63 (d, J=3.4 Hz, 1H), 7.52-7.33 (m, 1H), 7.23-7.01 (m, 5H), 5.07-4.95 (m, 2H), 4.53-4.44 (m, 1H), 3.23-3.05 (m, 3H), 3.03-2.89 (m, 3H), 2.79-2.64 (m, 3H), 2.42-2.19 (m, 3H), 2.16-1.93 (m, 1H), 1.73-1.54 (m, 2H), 1.45 (s, 3H), 1.32 (s, 9H), 1.22 (s, 3H).

### (1R,2S,3R,5R)-3-[4-Amino-5-(1,3-thiazol-2-yl)pyrrolo[2,3-d]pyrimidin-7-yl]-5-{[(3-{[2-(4-fluorophenyl)ethyl]amino}propyl)(1H-pyrazol-4-yl)amino]methyl}cyclopentane-1,2-diol (599)

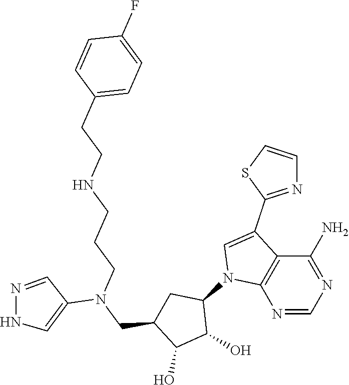 [SMILES: Nc1ncnc2c1c(-c1nccs1)cn2[C@@H]1C[C@H](CN(CCCNCCc2ccc(F)cc2)c2cn[nH]c2)[C@@H](O)[C@H]1O]

To tert-butyl-N-[3-({[(3aR,4R,6R,6aS)-6-[4-amino-5-(1,3-thiazol-2-yl)pyrrolo[2,3-d]pyrimidin-7-yl]-2,2-dimethyl-tetrahydro-3aH-cyclopenta[d][1,3]dioxol-4-yl]methyl}(1H-pyrazol-4-yl)amino)propyl]-N-[2-(4-fluorophenyl)ethyl]carbamate 185c (30 mg, 0.01 mmol, 1.0 eq) was added 4M HCl in 1,4-dioxane (2.0 mL, 8.00 mmol) and the reaction mixture was stirred for 2 h at room temperature. The resulting mixture was concentrated under vacuum and then dissolved in MeOH (2.0 mL) and adjusted to pH 8 with NH₃·H₂O. The crude residue was purified by Prep-HPLC [Column: YMC-Actus Triart C18, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 20% B to 60% B in 7 min, 55% B; Wave Length: 254 nm]to afford (1R,2S,3R,5R)-3-[4-amino-5-(1,3-thiazol-2-yl)pyrrolo[2,3-d]pyrimidin-7-yl]-5-{[(3-{[2-(4-fluorophenyl)ethyl]amino}propyl)(1H-pyrazol-4-yl)amino]methyl}cyclopentane-1,2-diol 599 (8.9 mg, 22%) as an off-white solid. LCMS [conditions Cortecs C18+, 50*3.0 mm, 2.7 μm, Mobile Phase A: Water/0.02% TFA, Mobile Phase B: Acetonitrile, 5%-50%-95% B-5 min (+), 1.50 L/min, ES, m/z]: T_{R}=2.19 min; [M+H]⁺: 592.1H NMR (300 MHZ, DMSO-d₆+D₂O) δ 8.12 (s, 1H), 8.07 (s, 1H), 7.79 (d, J=3.4 Hz, 1H), 7.57 (d, J=3.4 Hz, 1H), 7.24-7.13 (m, 4H), 7.09-6.97 (m, 2H), 4.96-4.81 (m, 1H), 4.45-4.32 (m, 1H), 3.83-3.73 (m, 1H), 3.20-3.06 (m, 1H), 3.05-2.84 (m, 3H), 2.63 (s, 3H), 2.45-2.379 (m, 2H), 2.31-2.10 (m, 2H), 1.67-1.47 (m, 3H), 1.25-1.09 (m, 1H).

### 3-{[2-(4-Fluorophenyl)ethyl]amino}propan-1-ol (186c) [commercially available]

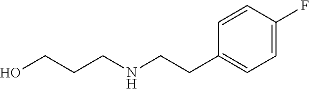 [SMILES: OCCCNCCc1ccc(F)cc1]

To a solution of 1-(2-bromoethyl)-4-fluorobenzene (4.00 g, 19.7 mmol, 1.0 eq) in ethanol (40 mL) was added propanolamine (2.96 g, 39.4 mmol, 2.0 eq) at room temperature and the reaction mixture was stirred at 80° C. for 6 h. The resulting mixture was concentrated under vacuum to afford 3-{[2-(4-fluorophenyl)ethyl]amino}propan-1-ol 186c (4.00 g, crude). The crude product was used in the next step directly without purification. LCMS [conditions Xbridge Shield C18, 50*3.0 mm, 3.5 μm, Mobile Phase A: Water/0.05% Ammonia, Mobile Phase B: Acetonitrile, 5%-100% B-2 min (+), 1.50 L/min, ES, m/z]: T_{R}=0.81 min; [M+H]⁺: 198.

### tert-Butyl-N-[2-(4-fluorophenyl)ethyl]-N-(3-hydroxypropyl)carbamate (187c)

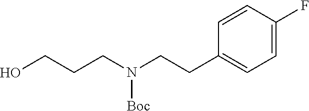 [SMILES: CC(C)(C)OC(=O)N(CCCO)CCc1ccc(F)cc1]

To a solution of 3-{[2-(4-fluorophenyl)ethyl]amino}propan-1-ol 186c (4.00 g, 20.3 mmol, 1.0 eq) in dichloromethane (40 mL) was added Boc₂O (6.64 g, 30.4 mmol, 1.5 eq) and Et₃N (6.16 g, 60.8 mmol, 3.0 eq) and the reaction mixture was stirred for 16 h at room temperature. The resulting mixture was diluted with water (20 mL) and extracted with dichloromethane (3×10 mL). The combined organic layers were washed with brine and dried over anhydrous Na₂SO₄. The filtrate was concentrated under reduced pressure and the crude residue was purified by silica gel column chromatography (70:30 Petroleum ether: tetrahydrofuran) to afford tert-butyl-N-[2-(4-fluorophenyl)ethyl]-N-(3-hydroxypropyl)carbamate 187c (4.00 g, 66%) as a pale yellow oil. LCMS [conditions XBridge C18, 50*3.0 mm, 3.5 μm, Mobile Phase A: Water/5 mM NH₄HCO₃, Mobile Phase B: Methanol, 10%-95% B-3 min (+), 1.50 L/min,ES, m/z]: T_{R}=1.85 min; [M+H]⁺: 298.

### tert-Butyl-N-[2-(4-fluorophenyl)ethyl]-N-(3-oxopropyl)carbamate (188c)

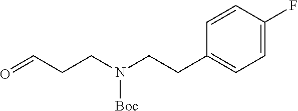 [SMILES: CC(C)(C)OC(=O)N(CCC=O)CCc1ccc(F)cc1]

To a solution of tert-butyl-N-[2-(4-fluorophenyl)ethyl]-N-(3-hydroxypropyl)carbamate 187c (0.50 g, 1.70 mmol, 1.0 eq) in DCM (15 mL) was added DMP (0.92 g, 2.20 mmol, 1.3 eq) in several portions over 5 min and the reaction mixture was stirred for 3 h at room temperature. The resulting mixture was concentrated under vacuum and purified by silica gel column chromatography (4:1 Petroleum ether: AcOEt) to afford tert-butyl-N-[2-(4-fluorophenyl)ethyl]-N-(3-oxopropyl)carbamate 188c (400 mg, 80%) as a colorless oil. LCMS [conditions XBridge C18, 50*3.0 mm, 3.5 μm, Mobile Phase A: Water/5 mM NH₄HCO₃, Mobile Phase B: Methanol, 10%-95% B-3 min (+), 1.50 L/min, ES, m/z]: 2.14 min; [M+H]⁺: 196. ¹H NMR (300 MHZ, DMSO-d₆) δ 9.65 (s, 1H), 7.28-7.18 (m, 2H), 7.17-7.06 (m, 2H), 3.45-3.28 (m, 4H), 2.74 (t, J=7.3 Hz, 2H), 2.66-2.55 (m, 2H), 1.34 (s, 9H).

### tert-Butyl-N-[2-(4-fluorophenyl)ethyl]-N-13-(1H-pyrazol-4-ylamino)propyl]carbamate (189c)

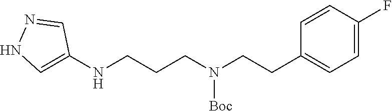 [SMILES: CC(C)(C)OC(=O)N(CCCNc1cn[nH]c1)CCc1ccc(F)cc1]

To a solution of 4-aminopyrazole hydrochloride (0.32 g, 2.70 mmol, 2.0 eq) in AcOH (0.08 g, 1.30 mmol, 1.0 eq) was added NaBH (OAc) 3 (0.15 g, 2.70 mmol, 2.0 eq) and the resulting mixture was stirred at 0° C. for 10 min. To the above mixture was added tert-butyl-N-[2-(4-fluorophenyl)ethyl]-N-(3-oxopropyl)carbamate 188c (0.40 g, 1.35 mmol, 1.0 eq) in DCM (3.0 mL), dropwise over 10 min, at 0° C. and the reaction mixture was stirred for 30 min at room temperature. The resulting mixture was concentrated under vacuum and purified by silica gel column chromatography (94:6 DCM: MeOH) to afford tert-butyl-N-[2-(4-fluorophenyl)ethyl]-N-[3-(1H-pyrazol-4-ylamino)propyl]carbamate 189c (400 mg, 80%) as a colorless oil. LCMS [conditions XBridge C18, 50*3.0 mm, 3.5 μm, Mobile Phase A: Water/5 mM NH₄HCO₃, Mobile Phase B: Methanol, 10%-95% B-3 min (+), 1.50 L/min,ES, m/z]: T_{R}=2.10; [M+H]⁺: 363.1H NMR (300 MHz, DMSO-d₆) δ7.27-7.17 (m, 2H), 7.17-7.06 (m, 2H), 7.04 (s, 2H), 3.39-3.26 (m, 2H), 3.22-3.16 (m, 2H), 2.84-2.69 (m, 4H), 1.76-1.61 (m, 2H), 1.35 (s, 9H).

### Synthesis of (1R,2S,3R,5R)-3-(4-Amino-5-(1-(methylsulfonyl)-1H-pyrazol-3-yl)-7H-pyrrolo[2,3-d]pyrimidin-7-yl)-5-(((3-(phenethylamino)propyl)amino)methyl)cyclopentane-1,2-diol (600)

### 1-(Methylsulfonyl)-3-(4,4,5,5-tetramethyl-1,3,2-dioxaborolan-2-yl)-1H-pyrazole (200c)

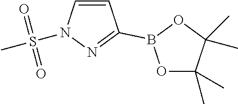

To a stirred solution of 3-(4,4,5,5-tetramethyl-1,3,2-dioxaborolan-2-yl)-1H-pyrazole (0.50 g, 2.60 mmol, 1.0 eq) and TEA (0.65 g, 6.40 mmol, 2.5 eq) in DCM (5.0 mL) was added MsCl (0.59 g, 5.15 mmol, 2.0 eq) in DCM (3.0 mL), dropwise, at room temperature and the reaction mixture was stirred for 4 h at room temperature. The reaction was quenched with saturated ammonium chloride solution and extracted with DCM (3×50 mL). The combined organic layers were washed with brine and dried over anhydrous Na₂SO₄. The filtrate was concentrated under reduced pressure to afford 1-methanesulfonyl-3-(4,4,5,5-tetramethyl-1,3,2-dioxaborolan-2-yl)pyrazole 200c (0.60 g, 51%) as a yellow solid. The crude product was used in the next step directly without further purification.

### tert-Butyl-(((3αR,4R,6R,6αS)-6-(4-amino-5-(1-(methylsulfonyl)-1H-pyrazol-3-yl)-7H-pyrrolo[2,3-d]pyrimidin-7-yl)-2,2-dimethyltetrahydro-4H-cyclopenta[d][1,3]dioxol-4-yl)methyl)(3-((tert-butoxycarbonyl)(phenethyl)amino)propyl)carbamate (201r)

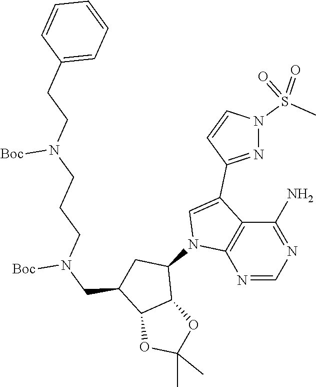 [SMILES: CC(C)(C)OC(=O)N(CCCN(C[C@H]1C[C@@H](n2cc(-c3ccn(S(C)(=O)=O)n3)c3c(N)ncnc32)[C@@H]2OC(C)(C)O[C@H]12)C(=O)OC(C)(C)C)CCc1ccccc1]

To a stirred solution of tert-butyl-N-[3-({[(3aR,4R,6R,6αS)-6-{4-amino-5-bromopyrrolo[2,3-d]pyrimidin-7-yl}-2,2-dimethyl-tetrahydro-3αH-cyclopenta[d][1,3]dioxol-4-yl]methyl}(tert-butoxycarbonyl)amino)propyl]-N-(2-phenylethyl)carbamate 108c (70 mg, 0.10 mmol, 1.0 eq) and 1-methanesulfonyl-3-(4,4,5,5-tetramethyl-1,3,2-dioxaborolan-2-yl)pyrazole 200c (0.13 g, 0.30 mmol, 3.0 eq) in 1,4-dioxane (1.0 mL) and H₂O (0.1 mL) were added Pd (DtBPF) Cl₂ (3.0 mg, 0.005 mmol, 0.05 eq) and K₃PO₄ (60 mg, 0.30 mmol, 3.0 eq) and the reaction mixture was stirred at 90° C. for 0.5 h, under N₂. The resulting mixture was concentrated under reduced pressure and extracted with AcOEt (3×20 mL). The combined organic layers were washed with brine, dried over anhydrous Na₂SO₄ and filtered. The filtrate was concentrated under reduced pressure and the crude residue was purified by Prep-TLC (60:40 Tetrahydrofuran: Petroleum ether) to afford tert-butyl-N-[3-({[(3aR,4R,6R,6aS)-6-[4-amino-5-(1-methanesulfonylpyrazol-3-yl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate 201c (70 mg, 88%) as a yellow solid. LCMS [conditions: XBridge C18, 50*3.0 mm, 3.5 μm, Mobile Phase A: Water/5 mM NH₄HCO₃, Mobile Phase B: Methanol, 10%-95% B-2 min (+).lcm, 1.50 mL/min]: T_{R}=1.11 min; [M+H]⁺: 809. ¹H NMR (300 MHZ, Chloroform-d) δ 8.25 (s, 1H), 8.06 (d, J=2.8 Hz, 1H), 7.70-7.48 (m, 1H), 7.36-7.27 (m, 2H), 7.26-7.14 (m, 3H), 7.00 (s, 1H), 6.81 (s, 2H), 4.98-4.82 (m, 1H), 4.66-4.53 (m, 1H), 4.09-3.90 (m, 1H), 3.48-3.43 (m, 2H), 3.34 (s, 3H), 3.25-3.13 (m, 2H), 2.94-2.74 (m, 2H), 2.58-2.35 (m, 3H), 2.16-1.69 (m, 6H), 1.57 (s, 3H), 1.46 (s, 18H), 1.30 (s, 3H).

### (1R,2S,3R,5R)-3-(4-Amino-5-(1-(methylsulfonyl)-1H-pyrazol-3-yl)-7H-pyrrolo[2,3-d]pyrimidin-7-yl)-5-(((3-(phenethylamino)propyl)amino)methyl)cyclopentane-1,2-diol (600)

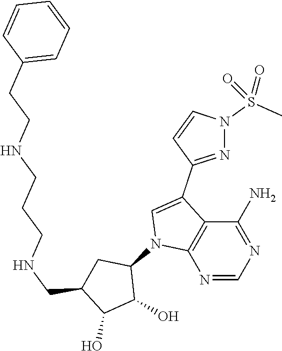 [SMILES: CS(=O)(=O)n1ccc(-c2cn([C@@H]3C[C@H](CNCCCNCCc4ccccc4)[C@@H](O)[C@H]3O)c3ncnc(N)c23)n1]

To tert-butyl-N-[3-({[(3aR,4R,6R,6aS)-6-[4-amino-5-(1-methanesulfonylpyrazol-3-yl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate 201c (70 mg, 0.10 mmol, 1.0 eq was added TFA (1.0 mL) and the reaction mixture was stirred at 50° C. for 1 h. The resulting mixture was concentrated under reduced pressure, dissolved in DMF (3.0 mL) and the solution was adjusted to pH 8 with NH₃·H₂O. The crude residue was purified by Prep-HPLC [Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 30 ml/min; Gradient: 25% B to 55% B in 7 min, 55% B; Wave Length: 220 nm]: to afford (1R,2S,3R,5R)-3-[4-amino-5-(1-methanesulfonylpyrazol-3-yl)pyrrolo[2,3-d]pyrimidin-7-yl]-5-[{{3-[(2-phenylethyl)amino]propyl}amino)methyl]cyclopentane-1,2-diol 600 (27.8 mg, 60%) as a white solid. LCMS [conditions: ZORBAX SB-Aq, 50*4.6 mm, 1.8 μm, Mobile Phase A: Water/0.05% TFA, Mobile Phase B: Acetonitrile, 5%-95% B-5 min, 1.20 mL/min]: T_{R}=2.10 min; [M+H]⁺: 569. ¹H NMR (400 MHZ, DMSO-06) 88.31 (d, J=2.9 Hz, 1H), 8.14-8.10 (s, 1H), 8.10 (s, 1H), 7.30-7.21 (m, 2H), 7.20-7.12 (m, 3H), 7.10 (d, J=2.9 Hz, 1H), 4.93 (dt, J=10.6, 8.3 Hz, 1H), 4.23 (dd, J=8.4, 5.6 Hz, 1H), 3.80 (dd, J=5.6, 3.8 Hz, 1H), 3.54 (s, 3H), 2.98-2.52 (m, 10H), 2.28-2.15 (m, 1H), 2.13-1.99 (m, 1H), 1.60-1.41 (m, 3H).

### Synthesis of (1R,2S,3R,5R)-3-(4-amino-5-(1,2,4-thiadiazol-5-yl)-7H-pyrrolo[2,3-d]pyrimidin-7-yl)-5-(((3-(phenethylamino)propyl)amino)methyl)cyclopentane-1,2-diol (601)

### tert-Butyl-(((3aR,4R,6R,6aS)-6-(4-amino-5-(4,4,5,5-tetramethyl-1,3,2-dioxaborolan-2-yl)-7H-pyrrolo[2,3-d]pyrimidin-7-yl)-2,2-dimethyltetrahydro-4H-cyclopenta[d][1,3]dioxol-4-yl)methyl)(3-((tert-butoxycarbonyl)(phenethyl)amino)propyl)carbamate (202c)

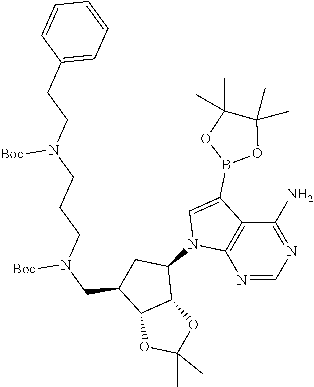 [SMILES: CC(C)(C)OC(=O)N(CCCN(C[C@H]1C[C@@H](n2cc(B3OC(C)(C)C(C)(C)O3)c3c(N)ncnc32)[C@@H]2OC(C)(C)O[C@H]12)C(=O)OC(C)(C)C)CCc1ccccc1]

To tert-butyl-N-[3-({[(3aR,4R,6R,6aS)-6-{4-amino-5-iodopyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 130c (100 mg, 0.11 mmol, 1.0 eq, 85% purity) in 1,4-dioxane (2.0 mL), was added bis(pinacolato)diboron (55 mg, 0.21 mmol, 2.0 eq), AcOK (32 mg, 0.32 mmol, 3.0 eq), Pd (dppf) Cl₂·CH₂Cl₂ (5.0 mg, 0.01 mmol, 0.05 eq), under N₂, and the reaction mixture was heated at 60° C. for 2 h. The resultant mixture was concentrated under reduced pressure, dissolved in DCM and the crude residue was purified by Prep-TLC (1:1 tetrahydrofuran: petroleum ether) to afford tert-butyl-N-[3-({[(3aR,4R,6R,6aS)-6-[4-amino-5-(4,4,5,5-tetramethyl-1,3,2-dioxaborolan-2-yl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate 202c (30 mg, 35%) as a yellow solid. LCMS [conditions: L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-2.5 min-1.2 mL-P.lcm, 1.20 mL/min]: T_{R}=2.17 min; [M+H]⁺: 791.

### tert-Butyl-(((3aR,4R,6R,6aS)-6-(4-amino-5-(1,2,4-thiadiazol-5-yl)-7H-pyrrolo[2,3-d]pyrimidin-7-yl)-2,2-dimethyltetrahydro-4H-cyclopenta[d][1,3]dioxol-4-yl)methyl)(3-((tert-butoxycarbonyl)(phenethyl)amino)propyl)carbamate (203c)

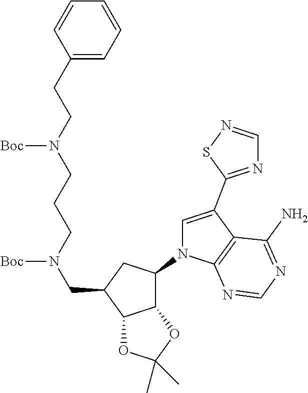 [SMILES: CC(C)(C)OC(=O)N(CCCN(C[C@H]1C[C@@H](n2cc(-c3ncns3)c3c(N)ncnc32)[C@@H]2OC(C)(C)O[C@H]12)C(=O)OC(C)(C)C)CCc1ccccc1]

To a stirred solution of tert-butyl-N-[3-({[(3aR,4R,6R,6aS)-6-[4-amino-5-(4,4,5,5-tetramethyl-1,3,2-dioxaborolan-2-yl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate 202c (30 mg, 0.04 mmol, 1.0 eq) and 5-bromo-1,2,4-thiadiazole (9.0 mg, 0.06 mmol, 1.5 eq) in 1,4-dioxane (1.0 mL) and H₂O (0.1 mL) were added Pd (dppf) Cl₂·CH₂Cl₂ (3.0 mg, 0.004 mmol, 0.1 eq) and K₃PO₄ (24 mg, 0.114 mmol, 3.0 eq) and the reaction mixture was stirred at 90° C. for 1 h, under nitrogen. The resulting mixture was concentrated under reduced pressure and partitioned between AcOEt and water. The aqueous layer was extracted with AcOEt (20 mL×2) and the combined organic layers were washed with brine, dried over anhydrous Na₂SO₄ and filtered. The filtrate was evaporated and the crude residue was purified by Prep-TLC (3:2 AcOEt: petroleum ether) to afford tert-butyl-N-[3-({[(3aR,4R,6R,6aS)-6-[4-amino-5-(1,2,4-thiadiazol-5-yl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate 203c (20 mg, 70%) as a yellow solid. LCMS [conditions: Kinetex XB-C18, 3.0*30 mm, 1.7 μm, Mobile Phase A: Water/0.1% FA, Mobile Phase B: Acetonitrile/0.05% FA, 5%-95% B-2 min (+).lcm, 1.20 mL/min]: T_{R}=1.40 min; [M+H]⁺: 749.

### (1R,2S,3R,5R)-3-(4-Amino-5-(1,2,4-thiadiazol-5-yl)-7H-pyrrolo[2,3-d]pyrimidin-7-yl)-5-(((3- (phenethylamino)propyl)amino)methyl)cyclopentane-1,2-diol (601)

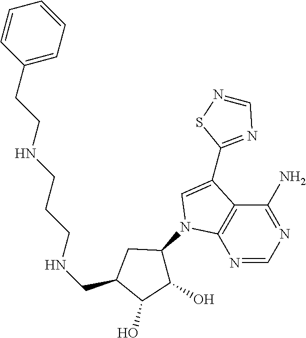 [SMILES: Nc1ncnc2c1c(-c1ncns1)cn2[C@@H]1C[C@H](CNCCCNCCc2ccccc2)[C@@H](O)[C@H]1O]

To tert-butyl-N-[3-({[(3aR,4R,6R,6aS)-6-[4-amino-5-(1,2,4-thiadiazol-5-yl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate 203c (20 mg, 0.02 mmol, 1.0 eq) was added TFA (0.5 mL) and the reaction mixture was stirred at room temperature for 2 h. The resulting mixture was concentrated under reduced pressure, dissolved in DMF (3.0 mL) and adjusted to pH 8 with NH₃·H₂O. The crude residue was purified by Prep-HPLC [Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 25% B to 50% B in 7 min, 50% B; Wave Length: 220 nm]: to afford (1R,2S,3R,5R)-3-[4-amino-5-(1,2,4-thiadiazol-5-yl)pyrrolo[2,3-d]pyrimidin-7-yl]-5-[{{3-[(2-phenylethyl)amino]propyl}amino)methyl]cyclopentane-1,2-diol 601 (2.2 mg, 32%) as a white solid. LCMS [conditions: ZORBAX SB-Aq, 50*4.6 mm, 1.8 μm, Mobile Phase A: Water/0.02% TFA, Mobile Phase B: Acetonitrile, 5%-95% B-5 min-1.5.lcm, 1.50 mL/min]: T_{R}=1.52 min; [M+H]⁺: 509. ¹H NMR (300 MHZ, DMSO-d₆) δ 8.82 (s, 1H), 8.58 (s, 1H), 8.16 (s, 1H), 7.31-7.07 (m, 5H), 5.03-4.88 (m, 1H), 4.30 (dd, J=8.7, 5.6 Hz, 1H), 3.83-3.77 (m, 1H), 3.04-2.46 (m, 10H), 2.28-2.15 (m, 1H), 2.11-2.05 (m, 1H), 1.61-1.55 (m, 3H).

### Synthesis of (1R,2S,3R,5R)-3-(4-amino-5-(thiazol-2-yl)-7H-pyrrolo[2,3-d]pyrimidin-7-yl)-5-(((3-((4-fluorophenethyl)amino)propyl)amino)methyl)cyclopentane-1,2-diol (602)

### tert-Butyl-N-[3-(1,3-dioxoisoindol-2-yl)propyl]-N-[2-(4-fluorophenyl)ethyl]carbamate (190c)

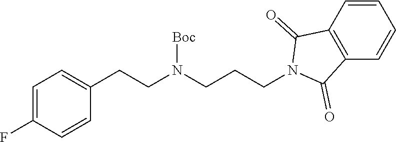 [SMILES: CC(C)(C)OC(=O)N(CCCN1C(=O)c2ccccc2C1=O)CCc1ccc(F)cc1]

To a solution of tert-butyl-N-[2-(4-fluorophenyl)ethyl]-N-(3-hydroxypropyl)carbamate 187c (1.40 g, 4.70 mmol, 1.0 eq) in dichloromethane (14 mL) was added PPh₃ (2.47 g, 9.40 mmol, 2.0 eq) and phthalimide (0.90 g, 6.12 mmol, 1.3 eq). To the resulting mixture was added DIAD (1.90 g, 9.4 mmol, 2.0 eq), dropwise over 10 min, at 0° C., under N₂ and the reaction mixture was stirred for 2 h at room temperature. The resulting mixture was concentrated under vacuum and the crude residue was purified by silica gel column chromatography (70:30 Petroleum ether: AcOEt) to afford tert-butyl-N-[3-(1,3-dioxoisoindol-2-yl)propyl]-N-[2-(4-fluorophenyl)ethyl]carbamate 190c (1.70 g, 85%) as a colorless oil. LCMS [conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-2 min (+), 1.50 L/min, ES, m/z]: T_{R}=1.62 min; [M+H]⁺: 427. ¹H NMR (300 MHZ, DMSO-d₆) δ 7.92-7.80 (m, 4H), 7.27-7.15 (m, 2H), 7.13-7.02 (m, 2H), 3.55 (t, J=7.0 Hz, 2H), 3.40-3.26 (m, 4H), 2.74 (t, J=7.4 Hz, 2H), 1.88-1.70 (m, 2H), 1.30 (s, 9H).

### tert-Butyl-N-(3-aminopropyl)-N-[2-(4-fluorophenyl]ethyl]carbamate (191r)-commercial

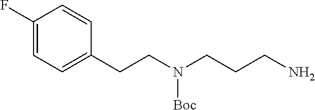 [SMILES: CC(C)(C)OC(=O)N(CCCN)CCc1ccc(F)cc1]

To a solution of tert-butyl-N-[3-(1,3-dioxoisoindol-2-yl)propyl]-N-[2-(4-fluorophenyl)ethyl]carbamate 190c (1.70 g, 4.00 mmol, 1.0 eq) in ethanol (17 mL) was added hydrazine hydrate (0.20 g, 8.00 mmol, 2.0 eq) and the reaction mixture was stirred at 80° C. for 4 h. The resultant mixture was allowed to cool to room temperature and the precipitate was filtered and washed with dichloromethane (3×10 mL). The combined filtrate was concentrated under vacuum to afford tert-butyl-N-(3-aminopropyl)-N-[2-(4-fluorophenyl)ethyl]carbamate 191c (1.10 g, 93%) as a colorless oil. LCMS [conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-2 min (+), 1.50 L/min, ES, m/z]: T_{R}=1.25 min; [M+H]⁺: 297. ¹H NMR (400 MHZ, DMSO-d₆) δ 7.27-7.19 (m, 2H), 7.16-7.07 (m, 2H), 3.35-3.27 (m, 2H), 3.20-3.13 (m, 2H), 2.79-2.71 (m, 2H), 2.50-2.44 (m, 2H), 1.56-1.45 (m, 2H), 1.36 (s, 9H).

### tert-Butyl-N-[3-({[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]methyl}amino)propyl]-N-[2-(4-fluorophenyl)ethyl]carbamate (192c)

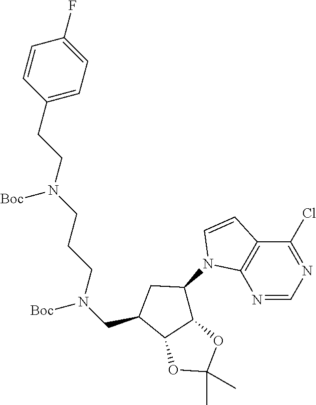 [SMILES: CC(C)(C)OC(=O)N(CCCN(C[C@H]1C[C@@H](n2ccc3c(Cl)ncnc32)[C@@H]2OC(C)(C)O[C@H]12)C(=O)OC(C)(C)C)CCc1ccc(F)cc1]

To a solution of tert-butyl-N-(3-aminopropyl)-N-[2-(4-fluorophenyl)ethyl]carbamate 191c (0.14 g, 0.50 mmol, 1.2 eq) in DCM (1.0 mL) was added HOAc (0.30 g, 4.0 mmol, 10.0 eq) and NaBH (OAc) 3 (0.17 g, 0.80 mmol, 2.0 eq) and the resulting mixture was stirred for 30 min at 0° C. To the above mixture was added (3aR,4S,6R,6aS)-6-{4-chloropyrrolo[2,3-d]pyrimidin-7-yl}-2,2-dimethyl-tetrahydro-3aH-cyclopenta[d][1,3]dioxole-4-carbaldehyde 77c (0.13 g, 0.40 mmol, 1.0 eq) in dichloromethane (1.0 mL), dropwise over 3 min, at 0° C. The resulting mixture was stirred for additional 30 min at 0° C. TEA (0.71 g, 6.00 mmol, 15 eq) and Boc₂O (0.18 g, 0.80 mmol, 2.0 eq) were added and the reaction mixture was stirred for 30 min and then concentrated under reduced pressure. The crude residue was dissolved in methanol (2.0 mL) and purified by Prep-HPLC [Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 30 ml/min; Gradient: 93% B to 93 B in 10 min, 93% B; Wave Length: 220 nm]: to afford tert-butyl-N-[3-({[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]methyl}amino)propyl]-N-[2-(4-fluorophenyl)ethyl]carbamate 192c (0.10 g, 41%) as a light yellow oil. LCMS [conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-2 min (+), 1.50 L/min, ES, m/z]: T_{R}=1.27 min; [M+H]⁺: 702.

### tert-Butyl-N-[3-({[(3aR,4R,6R,6aS)-6-{5-bromo-4-chloropyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-[2-(4-fluorophenyl)ethyl]carbamate (193c)

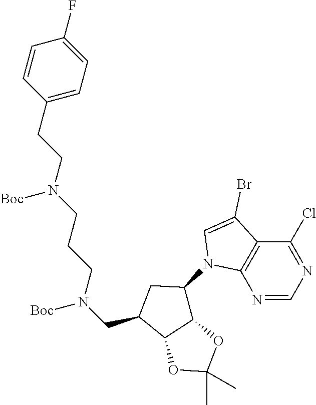 [SMILES: CC(C)(C)OC(=O)N(CCCN(C[C@H]1C[C@@H](n2cc(Br)c3c(Cl)ncnc32)[C@@H]2OC(C)(C)O[C@H]12)C(=O)OC(C)(C)C)CCc1ccc(F)cc1]

To a solution of tert-butyl-N-[3-({[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-[2-(4-fluorophenyl)ethyl]carbamate 192c (0.10 g, 0.14 mmol, 1.0 eq) in DMF (1.0 mL), was added NBS (35 mg, 0.20 mmol, 1.4 eq) in DMF (2.0 mL), dropwise over 3 min, at 0° C. and the reaction mixture was stirred for 2 h at room temperature. The resulting mixture was quenched by the addition of sat. Na₂S2O₃ aq. (3.0 ml) at 0° C. and extracted with AcOEt (3×5 mL). The combined organic layers were washed with sat. aq. NaHCO₃, brine and then dried over anhydrous Na₂SO₄. The filtrate was concentrated under reduced pressure and the crude residue was purified by silica gel column chromatography (60:40 Petroleum ether: AcOEt) to afford tert-butyl-N-[3-({[(3aR,4R,6R,6aS)-6-{5-bromo-4-chloropyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-[2-(4-fluorophenyl)ethyl]carbamate 193c (0.09 g, 81%) as a colorless oil. LCMS [conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min (+), 1.50 L/min, ES, m/z]: T_{R}=2.58 min; [M+H]⁺: 780, 782.1H NMR (300 MHZ, DMSO-d₆) δ 8.69 (s, 1H), 8.28 (s, 1H), 7.28-7.16 (m, 2H), 7.16-7.06 (m, 2H), 5.18-5.00 (m, 1H), 4.95-4.86 (m, 1H), 4.56-4.48 (m, 1H), 3.55-3.37 (m, 2H), 3.24-2.97 (m, 4H), 2.81-2.67 (m, 4H), 2.47-2.25 (m, 1H), 2.24-2.02 (m, 1H), 1.76-1.57 (m, 3H), 1.46 (s, 3H), 1.44-1.28 (m, 18H). 1.25 (s, 3H).

### tert-Butyl-N-[3-({[(3aR,4R,6R,6aS)-6-{4-amino-5-bromopyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]--[2-(4-fluorophenyl)ethyl]carbamate (194c)

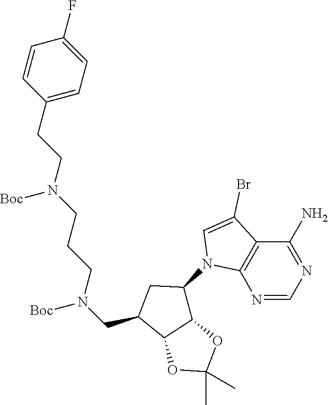 [SMILES: CC(C)(C)OC(=O)N(CCCN(C[C@H]1C[C@@H](n2cc(Br)c3c(N)ncnc32)[C@@H]2OC(C)(C)O[C@H]12)C(=O)OC(C)(C)C)CCc1ccc(F)cc1]

To tert-butyl-N-[3-({[(3aR,4R,6R,6aS)-6-{5-bromo-4-chloropyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-[2-(4-fluorophenyl)ethyl]carbamate 193c (0.09 g, 0.11 mmol, 1.0 eq) was added 7M NH₃ in methanol (2.0 mL, 14.0 mmol) and the reaction mixture was stirred at 80° C. for 16 h. The resulting mixture was concentrated under reduced pressure to afford tert-butyl-NV-[3-({[(3aR,4R,6R,6aS)-6-{4-amino-5-bromopyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-[2-(4-fluorophenyl)ethyl]carbamate 194c (90 mg, crude). The crude product was used in the next step directly without further purification. LCMS [conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min (+), 1.50 L/min, ES, m/z]: T_{R}=2.36 min; [M+H]⁺: 761, 763.

### tert-Butyl-N-[3-({[(3aR,4R,6R,6aS)-6-[4-amino-5-(1,3-thiazol-2-yl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-[2-(4-fluorophenyl)ethyl]carbamate (195c)

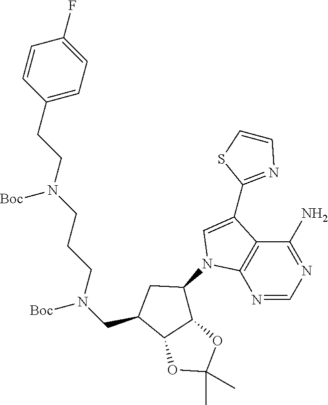 [SMILES: CC(C)(C)OC(=O)N(CCCN(C[C@H]1C[C@@H](n2cc(-c3nccs3)c3c(N)ncnc32)[C@@H]2OC(C)(C)O[C@H]12)C(=O)OC(C)(C)C)CCc1ccc(F)cc1]

To a solution of tert-butyl-N-[3-({[(3aR,4R,6R,6aS)-6-{4-amino-5-bromopyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-[2-(4-fluorophenyl)ethyl]carbamate 194c (0.09 g, 0.10 mmol, 1.0 eq) in toluene (1.0 mL) were added 2-(tributylstannyl)-1,3-thiazole (0.12 g, 0.30 mmol, 3.0 eq) and Pd (PPh₃) 2Cl₂ (7.8 mg, 0.01 mmol, 0.1 eq) and the reaction mixture was stirred at 120° C. for 2 h, under N₂. The resulting mixture was concentrated under vacuum and the crude residue was purified by silica gel column chromatography (60:40 Petroleum ether: AcOEt) to afford tert-butyl-N-[3-({[(3aR,4R,6R,6aS)-6-[4-amino-5-(1,3-thiazol-2-yl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-[2-(4-fluorophenyl)ethyl]carbamate 195c (50 mg, 58%) as a colorless oil. LCMS [conditions Xbridge Shield C18, 50*3.0 mm, 3.5 μm, Mobile Phase A: Water/0.05% Ammonia, Mobile Phase B: Acetonitrile 5%-100% B-3 min (+), 1.50 L/min, ES, m/z]: T_{R}=2.47 min; [M+H]⁺: 766.

### (1R,2S,3R,5R)-3-[4-Amino-5-(1,3-thiazol-2-yl)pyrrolo[2,3-d]pyrimidin-7-yl]-5-{[(3-{[2-(4-fluorophenyl)ethyl]amino}propyl)amino]methyl}cyclopentane-1,2-diol (602)

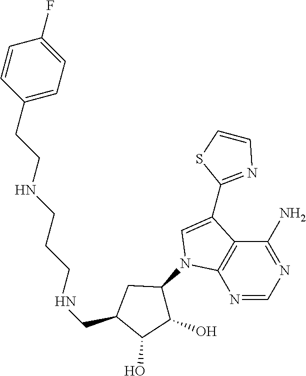 [SMILES: Nc1ncnc2c1c(-c1nccs1)cn2[C@@H]1C[C@H](CNCCCNCCc2ccc(F)cc2)[C@@H](O)[C@H]1O]

To tert-butyl-N-[3-({[(3aR,4R,6R,6aS)-6-[4-amino-5-(1,3-thiazol-2-yl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-[2-(4-fluorophenyl)ethyl]carbamate 195c (50 mg, 0.07 mmol, 1 eq) was added 4M HCl in MeOH (2.0 mL, 8.00 mmol) and the reaction mixture was stirred for 1 h at room temperature. The resulting mixture was concentrated under reduced pressure and the crude residue was dissolved in MeOH (2.0 mL) and the solution was adjusted to pH 8 with NH₃·H₂O. The crude mixture was purified by Prep-HPLC [Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 30 ml/min; Gradient: 20% B to 45% B in 8 min, Wave Length: 220 nm]: to afford (1R,2S,3R,5R)-3-[4-amino-5-(1,3-thiazol-2-yl)pyrrolo[2,3-d]pyrimidin-7-yl]-5-{[(3-{[2-(4-fluorophenyl)ethyl]amino}propyl)amino]methyl}cyclopentane-1,2-diol 602 (9.0 mg, 26%) as an off-white solid. LCMS [conditions ZORBAX SB-Aq, 50*4.6 mm, 1.8 μm, Mobile Phase A: Water/0.02% TFA, Mobile Phase B: Acetonitrile, 5%-95% B-5 min, 1.50 L/min, ES, m/z]: T_{R}=1.32 min; [M+H]⁺: 526. ¹H NMR (300 MHZ, DMSO-d₆) δ 8.14 (s, 1H), 8.09 (s, 1H), 7.81 (d, J=3.4 Hz, 1H), 7.59 (d, J=3.4 Hz, 1H), 7.25-7.13 (m, 2H), 7.09-6.99 (m, 2H), 4.91 (q, J=9.1 Hz, 1H), 4.33-4.23 (m, 1H), 3.82-3.72 (m, 1H), 2.76-2.53 (m, 10H), 2.32-2.12 (m, 1H), 2.12-1.98 (m, 1H), 1.65-1.43 (m, 3H).

### Synthesis of (1R,2S,3R,5R)-3-{4-Aminopyrrolo[2,3-d]pyrimidin-7-yl}-5-{[(3-bromo-2H-pyrazol-4-yl)(3-{[2-(4-fluorophenyl)ethyl]amino}propyl)amino]methyl}cyclopentane-1,2-diol (603)

### tert-Butyl-N-[3-({[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]methyl}(1H-pyrazol-4-yl)amino)propyl]-N-[2-(4-fluorophenyl)ethyl]carbamate (196c)

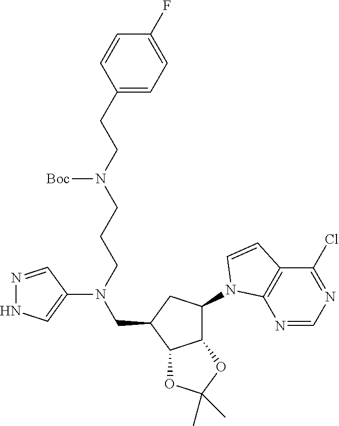 [SMILES: CC(C)(C)OC(=O)N(CCCN(C[C@H]1C[C@@H](n2ccc3c(Cl)ncnc32)[C@@H]2OC(C)(C)O[C@H]12)c1cn[nH]c1)CCc1ccc(F)cc1]

To a solution of tert-butyl-N-[2-(4-fluorophenyl)ethyl]-N-[3-(1H-pyrazol-4-ylamino)propyl]carbamate 189c (0.20 g, 0.60 mmol, 1.0 eq) in DCM (1.0 mL) was added HOAc (0.33 g, 5.50 mmol, 10 eq) and NaBH (OAc) 3 (0.23 g, 1.10 mmol, 2.0 eq) were added and the resulting mixture was stirred at 0° C. for 30 min. To the above mixture was added (3aR,4S,6R,6aS)-6-{4-chloropyrrolo[2,3-d]pyrimidin-7-yl}-2,2-dimethyl-tetrahydro-3aH-cyclopenta[d][1,3]dioxole-4-carbaldehyde 77c (0.21 g, 0.70 mmol, 1.2 eq) in DCM (1.0 mL), dropwise over 3 min, at 0° C. and the reaction mixture was stirred at 0° C. for 30 min. The resulting mixture was concentrated under reduced pressure and the crude residue purified by Prep-HPLC [Column: Welch Xtimate C18 ExRS, 250 mm, 10 μm; Mobile Phase A: Water (0.05% NH₃·H₂O), Mobile Phase B: ACN; Flow rate: 90 mL/min; Gradient: 90% B to 90% B in 10 min, 90% B; Wave Length: 254 nm]: to afford tert-butyl-N-[3-({[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]methyl}(1H-pyrazol-4-yl)amino)propyl]-N-[2-(4-fluorophenyl)ethyl]carbamate 196c (220 mg, 60%) as an off-white solid. LCMS [conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-2 min (+), 1.50 L/min, ES, m/z]: T_{R}=1.85 min; [M+H]⁺: 668. ¹H NMR (300 MHZ, DMSO-d₆) δ 12.25 (s, 1H), 8.64 (s, 1H), 7.96 (d, J=3.7 Hz, 1H), 7.22-7.13 (m, 4H), 7.12-7.02 (m, 2H), 6.76-6.69 (m, 1H), 5.14-5.02 (m, 1H), 4.95 (t, J=6.6 Hz, 1H), 4.54-4.44 (m, 1H), 3.33-3.24 (m, 2H), 3.12 (d, J=7.1 Hz, 3H), 3.04-2.92 (m, 3H), 2.70 (d, J=7.4 Hz, 2H), 2.35 (s, 2H), 2.16-2.03 (m, 1H), 1.67-1.59 (m, 2H), 1.46 (s, 3H), 1.32 (s, 9H), 1.21 (s, 3H).

### tert-Butyl-N-[3-({[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]methyl}(3-bromo-2H-pyrazol-4-yl)amino)propyl]-N-[2-(4-fluorophenyl)ethyl]carbamate (197c)

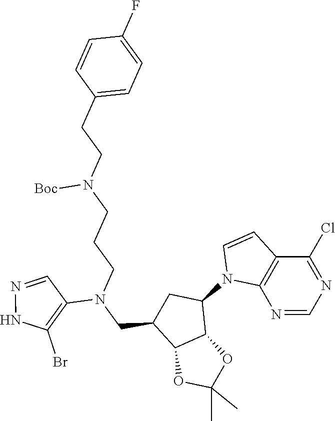 [SMILES: CC(C)(C)OC(=O)N(CCCN(C[C@H]1C[C@@H](n2ccc3c(Cl)ncnc32)[C@@H]2OC(C)(C)O[C@H]12)c1cn[nH]c1Br)CCc1ccc(F)cc1]

To a solution of tert-butyl-N-[3-({[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]methyl}(1H-pyrazol-4-yl)amino)propyl]-N-[2-(4-fluorophenyl)ethyl]carbamate 196c (0.20 g, 0.30 mmol, 1.0 eq) in DMF (1.0 mL) was added NBS (0.04 g, 0.2 0 mmol, 0.7 eq) in DMF (1.0 mL), dropwise over 10 min, at 0° C. and the reaction mixture was stirred at 0° C. for 5 min. The reaction was quenched by the addition of sat. aq. NaS₂O₃ (3.0 mL) at 0° C. and then extracted with AcOEt (3×3 mL). The combined organic layers were washed with sat. aq. NaHCO₃, brine and then dried over anhydrous Na₂SO₄. The filtrate was concentrated under reduced pressure and the crude residue was purified by silica gel column chromatography (70:30 Petroleum ether: AcOEt) to afford tert-butyl-N-[3-({[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]methyl}(3-bromo-2H-pyrazol-4-yl)amino)propyl]-N-[2-(4-fluorophenyl)ethyl]carbamate 197c (110 mg, 49%) as a white solid. LCMS [conditions XBridge C18, 50*3.0 mm, 3.5 μm, Mobile Phase A: Water/5 mM NH₄HCO₃, Mobile Phase B: Methanol, 10%-95% B-3 min (+), 1.50 L/min, ES, m/z]: T_{R}=2.42 min; [M+H]⁺: 746, 748.1H NMR (300 MHZ, DMSO-d₆) δ 8.65 (s, 1H), 7.98 (s, 1H), 7.24-7.18 (m, 3H), 7.17-7.08 (m, 2H), 6.74 (d, J=3.7 Hz, 1H), 5.11-5.05 (m, 1H), 4.95 (t, J=6.4 Hz, 1H), 4.59-4.53 (m, 1H), 3.13-3.07 (m, 7H), 2.78-2.72 (m, 3H), 2.42-1.97 (m, 3H), 1.72-1.64 (m, 2H), 1.46 (s, 3H), 1.38 (s, 9H), 1.22 (s, 3H).

### tert-Butyl-NV-[3-({[(3aR,4R,6R,6aS)-6-{4-aminopyrrolo[2,3-d]pyrimidin-7-yl}-2,2-dimethyl-tetrahydro-3aH-cyclopenta[d][1,3]dioxol-4-yl]methyl}(3-bromo-2H-pyrazol-4-yl)amino)propyl]-N-[2-(4-fluorophenyl)ethyl]carbamate (198c)

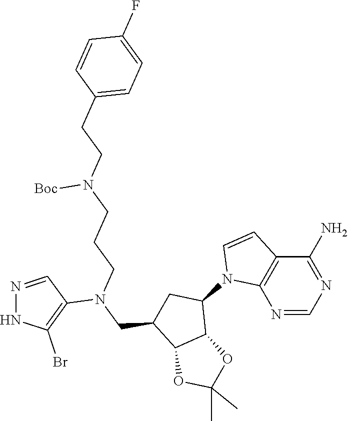 [SMILES: CC(C)(C)OC(=O)N(CCCN(C[C@H]1C[C@@H](n2ccc3c(N)ncnc32)[C@@H]2OC(C)(C)O[C@H]12)c1cn[nH]c1Br)CCc1ccc(F)cc1]

To tert-butyl-N-[3-({[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]methyl}(3-bromo-2H-pyrazol-4-yl)amino)propyl]-N-[2-(4-fluorophenyl)ethyl]carbamate 197c (0.11 g, 0.10 mmol, 1.0 eq) was added 7M NH₃ in methanol (2.0 mL, 14.0 mmol) and the reaction mixture was stirred at 80° C. for 16 h. The resulting mixture was concentrated under reduced pressure and the crude residue was purified by silica gel column chromatography (93:7 DCM: MeOH) to afford tert-butyl-N-[3-({[(3aR,4R,6R,6aS)-6-{4-aminopyrrolo[2,3-d]pyrimidin-7-yl}-2,2-dimethyl-tetrahydro-3aH-cyclopenta[d][1,3]dioxol-4-yl]methyl}(3-bromo-2H-pyrazol-4-yl)amino)propyl]-N-[2-(4-fluorophenyl)ethyl]carbamate 198c (50 mg, 47%) as an off-white solid. LCMS [conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-2 min (+), 1.50 L/min, ES, m/z]: T_{R}=1.49 min; [M+H]⁺: 727, 729.

### (1R,2S,3R,5R)-3-{4-Aminopyrrolo[2,3-d]pyrimidin-7-yl}-5-{[(3-bromo-2H-pyrazol-4-yl)(3-{[2-(4-fluorophenyl)ethyl]amino}propyl)amino]methyl}cyclopentane-1,2-diol (603)

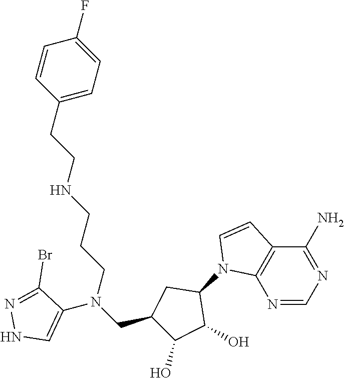 [SMILES: Nc1ncnc2c1ccn2[C@@H]1C[C@H](CN(CCCNCCc2ccc(F)cc2)c2c[nH]nc2Br)[C@@H](O)[C@H]1O]

To a solution of tert-butyl-N-[3-({[(3aR,4R,6R,6aS)-6-{4-aminopyrrolo[2,3-d]pyrimidin-7-yl}-2,2-dimethyl-tetrahydro-3aH-cyclopenta[d][1,3]dioxol-4-yl]methyl}(3-bromo-2H-pyrazol-4-yl)amino)propyl]-N-[2-(4-fluorophenyl)ethyl]carbamate 198c (0.05 g, 0.10 mmol, 1.0 eq) in DCM (1.0 mL) was added 4M HCl in MeOH (1.0 mL, 4.00 mmol) and the reaction mixture was stirred for 1 h at room temperature. The resulting mixture was concentrated under reduced pressure and adjusted to pH 8 with NH₃·H₂O. The crude mixture was purified by Prep-HPLC [Column: YMC-Actus Triart C18, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 24% B to 49% B in 7 min, 49% B; Wave Length: 220 nm]: to afford (1R,2S,3R,5R)-3-{4-aminopyrrolo[2,3-d]pyrimidin-7-yl}-5-{[(3-bromo-2H-pyrazol-4-yl)(3-{[2-(4-fluorophenyl)ethyl]amino}propyl)amino]methyl}cyclopentane-1,2-diol 603 (19.3 mg, 48%) as an off-white solid. LCMS [conditions ZORBAX SB-Aq, 50*4.6 mm, 1.8 μm, Mobile Phase A: Water/0.02% TFA, Mobile Phase B: Acetonitrile, 5%-95% B-3 min (+), 1.50 L/min, ES, m/z]: T_{R}=1.14 min; [M+H]⁺: 587. ¹H NMR (300 MHZ, DMSO-d₆) δ 7.99 (s, 1H), 7.56 (s, 1H), 7.22 (d, J=3.6 Hz, 1H), 7.18-7.10 (m, 2H), 7.08-6.95 (m, 2H), 6.55 (d, J=3.6 Hz, 1H), 4.84-4.67 (m, 1H), 4.25-4.12 (m, 1H), 3.74-3.60 (m, 1H), 3.16-3.01 (m, 1H), 2.96-2.67 (m, 3H), 2.64-2.56 (m, 4H), 2.57-2.51 (m, 2H), 2.36-2.06 (m, 1H), 2.04-1.91 (m, 1H), 1.60-1.37 (m, 3H).

### Synthesis of (1R,2S,3R,5R)-3-(4-amino-5-(1-(phenylsulfonyl)-1H-pyrazol-3-yl)-7H-pyrrolo[2,3-d]pyrimidin-7-yl)-5-(((3-(phenethylamino)propyl)amino)methyl)cyclopentane-1,2-diol. (604)

### (1-(Phenylsulfonyl)-1H-pyrazol-3-yl) boronic acid (204c)

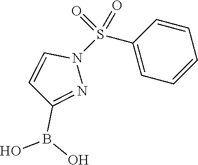 [SMILES: O=S(=O)(c1ccccc1)n1ccc(B(O)O)n1]

To a stirred suspension of 3-(4,4,5,5-tetramethyl-1,3,2-dioxaborolan-2-yl)-1H-pyrazole (500 mg, 2.60 mmol, 1.0 eq) and TEA (522 mg, 5.20 mmol, 2.0 eq) in DCM (5 mL) was added benzenesulfonyl chloride (683 mg, 3.80 mmol, 1.5 eq) in DCM (1.0 mL) at room temperature and the reaction mixture was stirred for 4 h at room temperature. The resulting mixture was concentrated under reduced pressure and the crude residue was purified by reverse phase flash (Column: C18, Mobile Phase A: H₂O, Mobile Phase B: ACN; Flow rate: 70 mL/min; Gradient: 5% B to 25% B in 15 min): to afford 1-(benzenesulfonyl)pyrazol-3-ylboronic acid 204c (300 mg, 51%) as a yellow solid.

### tert-Butyl-(((3aR,4R,6R,6aS)-6-(4-amino-5-(1-(phenylsulfonyl)-1H-pyrazol-3-yl)-7H-pyrrolo[2,3-d]pyrimidin-7-yl)-2,2-dimethyltetrahydro-4H-cyclopenta[d][1,3]dioxol-4-yl)methyl)(3-((tert-butoxycarbonyl)(phenethyl)amino)propyl)carbamate (205c)

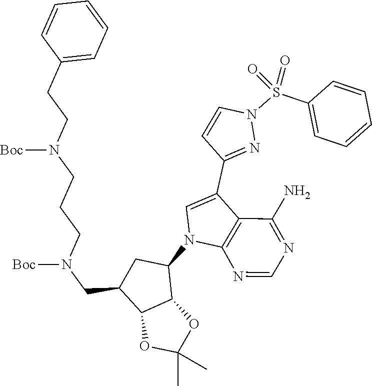 [SMILES: CC(C)(C)OC(=O)N(CCCN(C[C@H]1C[C@@H](n2cc(-c3ccn(S(=O)(=O)c4ccccc4)n3)c3c(N)ncnc32)[C@@H]2OC(C)(C)O[C@H]12)C(=O)OC(C)(C)C)CCc1ccccc1]

To a stirred suspension of tert-butyl-N-[3-({[(3aR,4R,6R,6aS)-6-{4-amino-5-bromopyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 108c (70 mg, 0.09 mmol, 1.0 eq) and 1-(benzenesulfonyl)pyrazol-3-ylboronic acid 204c (71 mg, 0.28 mmol, 3.0 eq) in dioxane (1.0 mL) and H₂O (0.1 mL), under N₂, were added Pd (dppf) Cl₂·CH₂Cl₂ (7.7 mg, 0.009 mmol, 0.1 eq) and K₃PO₄ (60 mg, 0.28 mmol, 3.0 eq) and the reaction mixture was stirred at 90° C. for 1 h. The resulting mixture was concentrated under reduced pressure, dissolved in AcOEt, washed with brine and then dried over anhydrous Na₂SO₄. The filtrate was concentrated under reduced pressure and the crude residue was purified by Prep-TLC (3:2 Tetrahydrofuran: petroleum ether) to afford tert-butyl-N-[3-({[(3aR,4R,6R,6aS)-6-{4-amino-5-[1-(benzenesulfonyl)pyrazol-3-yl]pyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 205c (70 mg, 85%) as a light yellow solid. LCMS [conditions: ZORBAX SB-Aq, 50*4.6 mm, 1.8 μm, Mobile Phase A: Water/0.02% TFA, Mobile Phase B: Acetonitrile, 5%-50%-95% B-5 min-1.5.lcm, 1.50 mL/min]: T_{R}=1.91 min; [M+H]⁺: 871.

### (1R,2S,3R,5R)-3-(4-Amino-5-(1-(phenylsulfonyl)-1H-pyrazol-3-yl)-7H-pyrrolo[2,3-d]pyrimidin-7-yl)-5-(((3-(phenethylamino)propyl)amino)methyl)cyclopentane-1,2-diol. (604)

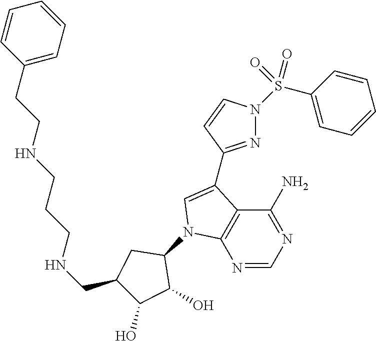 [SMILES: Nc1ncnc2c1c(-c1ccn(S(=O)(=O)c3ccccc3)n1)cn2[C@@H]1C[C@H](CNCCCNCCc2ccccc2)[C@@H](O)[C@H]1O]

To tert-butyl-N-[3-({[(3aR,4R,6R,6aS)-6-{4-amino-5-[1-(benzenesulfonyl)pyrazol-3-yl]pyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 205c (70 mg, 0.08 mmol, 1.0 eq) was added TFA (1.0 mL) and the reaction mixture was stirred at 50° C. for 1 h. The resulting mixture was concentrated under reduced pressure and adjusted to pH 8 with NH₃·H₂O. The crude residue was purified by Prep-HPLC [Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 20% B to 55% B in 7 min, 55% B; Wave Length: 220 nm]: to afford (1R,2S,3R,5R)-3-{4-amino-5-[1-(benzenesulfonyl)pyrazol-3-yl]pyrrolo[2,3-d]pyrimidin-7-yl}-5-[{{3-[(2-phenylethyl)amino]propyl}amino)methyl]cyclopentane-1,2-diol 604 (22.4 mg, 44%) as a white solid. LCMS [conditions: ZORBAX SB-Aq, 50*4.6 mm, 1.8 μm, Mobile Phase A: Water/0.02% TFA, Mobile Phase B: Acetonitrile, 5%-50%-95% B-5 min-1.5.lcm, 1.50 mL/min]: T_{R}=1.18 min; [M+H]⁺: 631. ¹H NMR (400 MHZ, DMSO-d₆) δ 8.50 (d, J=2.9 Hz, 1H), 8.06 (d, J=4.2 Hz, 2H), 8.01-7.94 (m, 2H), 7.84-7.75 (m, 1H), 7.73-7.65 (m, 2H), 7.27-7.19 (m, 2H), 7.19-7.10 (m, 3H), 7.08 (d, J=2.9 Hz, 1H), 4.94-4.82 (m, 1H), 4.22-4.14 (m, 1H), 3.80-3.73 (m, 1H), 2.78-2.51 (m, 10H), 2.24-2.12 (m, 1H), 2.03 (br s, 1H), 1.57-1.51 (m, 2H), 1.50-1.39 (m, 1H).

### Synthesis of (1R,2S,3R,5R)-3-(4-amino-5-(4-benzylthiazol-2-yl)-7H-pyrrolo[2,3-d]pyrimidin-7-yl)-5-(((3-(phenethylamino)propyl)amino)methyl)cyclopentane-1,2-diol (605)

### tert-Butyl-(3-((((3aR,4R,6R,6aS)-6-(4-amino-5-(4-benzylthiazol-2-yl)-7H-pyrrolo[2,3-d]pyrimidin-7-yl)-2,2-dimethyltetrahydro-4H-cyclopenta[d][1,3]dioxol-4-yl)methyl)(tert-butoxycarbonyl)amino)propyl)(phenethyl)carbamate (206c)

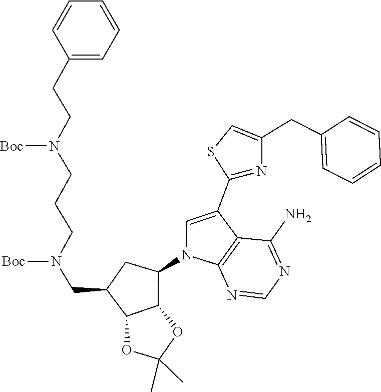 [SMILES: CC(C)(C)OC(=O)N(CCCN(C[C@H]1C[C@@H](n2cc(-c3nc(Cc4ccccc4)cs3)c3c(N)ncnc32)[C@@H]2OC(C)(C)O[C@H]12)C(=O)OC(C)(C)C)CCc1ccccc1]

To tert-butyl-NV-[3-({[(3aR,4R,6R,6aS)-6-{4-amino-5-iodopyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 130c (40 mg, 0.05 mmol, 1.0 eq) in toluene (0.5 mL) was added Pd (PPh₃) 2Cl₂ (1.8 mg, 0.003 mmol, 0.05 eq) and 4-benzyl-2-(tributylstannyl)-1,3-thiazole (70.5 mg, 0.15 mmol, 3.0 eq) at room temperature and the reaction mixture was stirred at 130° C. for 3 h, under N₂. The resulting mixture was extracted with AcOEt (3×10 mL) and the combined organic layers were washed with brine and dried over anhydrous Na₂SO₄. The filtrate was concentrated under reduced pressure and the crude residue was purified by silica gel column chromatography (3:2 tetrahydrofuran: petroleum ether) to afford tert-butyl-N-[3-({[(3aR,4R,6R,6aS)-6-[4-amino-5-(4-benzyl-1,3-thiazol-2-yl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate 206c (30.0 mg, 70%) as a yellow oil. LCMS [conditions: ZORBAX SB-Aq, 50*4.6 mm, 1.8 μm, Mobile Phase A: Water/0.02% TFA, Mobile Phase B: Acetonitrile, 5%-95% B-3 min-1.5.lcm, 1.50 mL/min]: T_{R}=1.91 min; [M+H]⁺: 838.

### (1R,2S,3R,5R)-3-(4-Amino-5-(4-benzylthiazol-2-yl)-7H-pyrrolo[2,3-d]pyrimidin-7-yl)-5-(((3-(phenethylamino)propyl)amino)methyl)cyclopentane-1,2-diol (605)

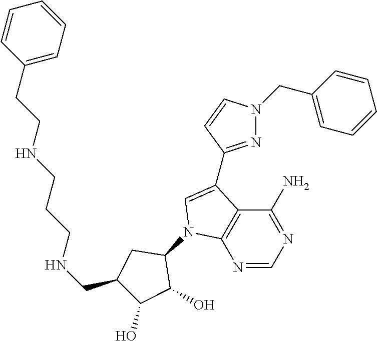 [SMILES: Nc1ncnc2c1c(-c1ccn(Cc3ccccc3)n1)cn2[C@@H]1C[C@H](CNCCCNCCc2ccccc2)[C@@H](O)[C@H]1O]

To tert-butyl-N-[3-({[(3aR,4R,6R,6aS)-6-[4-amino-5-(4-benzyl-1,3-thiazol-2-yl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate 206c (25 mg, 0.03 mmol, 1.0 eq) was added TFA (1 mL) and the reaction mixture was stirred at 50° C. for 1 h. The resulting mixture was allowed to cool, concentrated under reduced pressure and adjusted to pH 8 with NH₃·H₂O. The crude residue was purified by Prep-HPLC [Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 20% B to 55% B in 7 min, 55% B; Wave Length: 220 nm]: to afford (1R,2S,3R,5R)-3-[4-amino-5-(4-benzyl-1,3-thiazol-2-yl)pyrrolo[2,3-d]pyrimidin-7-yl]-5-[{{3-[(2-phenylethyl)amino]propyl}amino)methyl]cyclopentane-1,2-diol 605 (6.4 mg, 36%) as a white solid. LCMS [conditions: ZORBAX SB-Aq, 50*4.6 mm, 1.8 μm, Mobile Phase A: Water/0.02% TFA, Mobile Phase B: Acetonitrile, 5%-95% B-3 min-1.5.lcm, 1.50 mL/min]: T_{R}=1.22 min; [M+H]⁺: 598. 1H NMR (400 MHZ, DMSO-d₆) δ 8.08 (s, 1H), 8.07 (s, 1H), 7.34-7.31 (m, 4H), 7.26-7.19 (m, 4H), 7.18-7.11 (m, 3H), 4.93-4.82 (m, 1H), 4.29-4.21 (m, 1H), 4.05 (s, 2H), 3.80-3.73 (m, 1H), 2.76-2.64 (m, 5H), 2.62-2.53 (m, 5H), 2.24-2.12 (m, 1H), 2.04 (s, 1H), 1.61-1.49 (s, 3H).

### Synthesis of (1S,2R,3R,5R)-3-{[(3-aminopropyl)(3-bromo-2H-pyrazol-4-yl)amino]methyl}-5-[4-(methylamino)pyrrolo[2,3-d]pyrimidin-7-yl]cyclopentane-1,2-diol (606)

### tert-Butyl-N-[3-({[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]methyl}(1H-pyrazol-4-yl)amino)propyl]carbamate (216c)

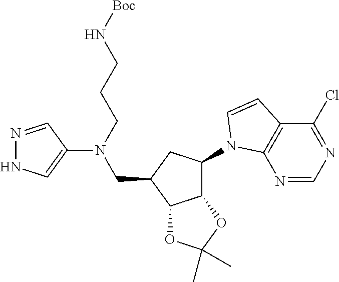 [SMILES: CC(C)(C)OC(=O)NCCCN(C[C@H]1C[C@@H](n2ccc3c(Cl)ncnc32)[C@@H]2OC(C)(C)O[C@H]12)c1cn[nH]c1]

To a solution of N-{[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]methyl}-1H-pyrazol-4-amine 212c (0.11 g, 0.28 mmol, 1.0 eq) in DCM (1.0 mL) were added NaBH (OAc) 3 (0.12 g, 0.57 mmol, 2.0 eq) and HOAc (0.17 g, 2.83 mmol, 10.0 eq) and the resulting mixture was stirred at 0° C. for 10 min. To the above mixture was added tert-butyl-N-(3-oxopropyl)carbamate (73 g, 0.42 mmol, 1.5 eq) in DCM (1.0 mL), dropwise over 3 min, at 0° C. and the reaction mixture was stirred at 0° C. for 30 min. The resulting mixture was concentrated under reduced pressure and the crude residue was adjusted to pH 8 with TEA and then purified by silica gel column chromatography (96:4 DCM: MeOH) to afford tert-butyl-N-[3-({[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]methyl}(1H-pyrazol-4-yl)amino)propyl]carbamate 216c (110 mg, 71%) as a colorless oil. LCMS [conditions Xbridge Shield C18, 50*3.0 mm, 3.5 μm, Mobile Phase A: Water/0.05% Ammonia, Mobile Phase B: Acetonitrile, 10%-95% B-3 min (+), 1.50 L/min, ES, m/z]: T_{R}=1.29 min; [M+H]⁺: 546. ¹H NMR (300 MHZ, DMSO-d₆) δ 12.03 (s, 1H), 8.65 (s, 1H), 7.97 (d, J=3.7 Hz, 1H), 7.18 (s, 1H), 6.81 (s, 1H), 6.73 (d, J=3.7 Hz, 1H), 5.13-5.01 (m, 1H), 5.01-4.91 (m, 1H), 4.54-4.46 (m, 1H), 4.41-4.32 (m, 1H), 3.43-3.36 (m, 3H), 3.20-3.07 (m, 1H), 3.04-2.89 (m, 4H), 2.45-2.25 (m, 1H), 2.15-1.97 (m, 1H), 1.48 (s, 3H), 1.38 (s, 9H), 1.22 (s, 3H).

### tert-Butyl-N-[3-({[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]methyl}(3-bromo-2H-pyrazol-4-yl)amino)propyl]carbamate (217c)

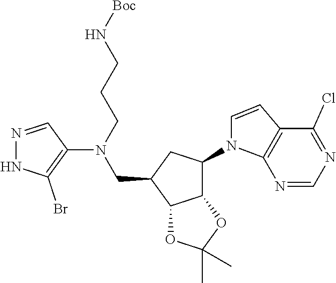 [SMILES: CC(C)(C)OC(=O)NCCCN(C[C@H]1C[C@@H](n2ccc3c(Cl)ncnc32)[C@@H]2OC(C)(C)O[C@H]12)c1cn[nH]c1Br]

To a solution of tert-butyl-N-[3-({[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]methyl}(1H-pyrazol-4-yl)amino)propyl]carbamate 216c (0.11 g, 0.20 mmol, 1.0 eq) in DMF (1.0 mL), at 0° C., was added NBS (25 mg, 0.14 mmol, 0.7 eq) in DMF (1.0 mL), dropwise over 10 min and the resulting mixture was stirred for 5 min at 0° C. The reaction was quenched by the addition of saturated aq. NaS2O₃ (3.0 mL) at 0° C. and the resulting mixture was extracted with AcOEt (3×3 mL). The combined organic layers were washed with brine, dried over anhydrous Na₂SO₄ and the filtrate was concentrated under reduced pressure. The crude residue was purified by silica gel column chromatography (94:6 DCM: MeOH) to afford tert-butyl-N-[3-({[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]methyl}(3-bromo-2H-pyrazol-4-yl)amino)propyl]carbamate 217c (50 mg, 40%) as a colorless semi-solid. LCMS [conditions L-column3 ODS, 50*3.0 mm, 3.0 μm, Mobile Phase A: Water/5 mM NH₄HCO₃+0.05% Ammonia, Mobile Phase B: Acetonitrile/5% water, 10%-95% B-3 min (+), 1.50 L/min, ES, m/z]: T_{R}=2.16 min; [M+H]⁺: 624, 626.1H NMR (300 MHZ, DMSO-d₆) δ 12.85 (s, 1H), 8.63 (d, J=6.2 Hz, 1H), 7.96 (d, J=3.6 Hz, 1H), 7.65 (s, 1H), 6.78-6.59 (m, 1H), 5.13-4.97 (m, 1H), 4.98-4.81 (m, 1H), 4.50 (s, 1H), 4.41-4.33 (m, 1H), 4.12-3.95 (m, 1H), 3.49-3.37 (m, 2H), 3.22-3.09 (m, 1H), 3.05-2.79 (m, 4H), 2.42-2.02 (m, 1H), 1.62-1.48 (m, 1H), 1.44 (s, 3H), 1.38 (s, 9H), 1.22 (s, 3H).

### tert-Butyl-N-[3-({[(3aR,4R,6R,6aS)-2,2-dimethyl-6-[4-(methylamino)pyrrolo[2,3-d]pyrimidin-7-yl]-tetrahydro-3aH-cyclopenta[d][1,3]dioxol-4-yl]methyl}(3-bromo-2H-pyrazol-4-yl)amino)propyl]carbamate (218c)

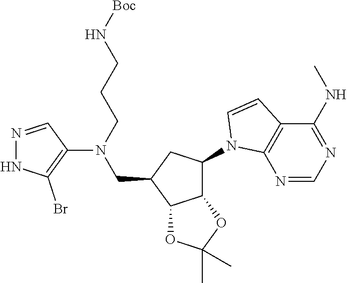 [SMILES: CNc1ncnc2c1ccn2[C@@H]1C[C@H](CN(CCCNC(=O)OC(C)(C)C)c2cn[nH]c2Br)[C@H]2OC(C)(C)O[C@H]21]

To tert-butyl-N-[3-({[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]methyl}(3-bromo-2H-pyrazol-4-yl)amino)propyl]carbamate 217c (50 mg, 0.08 mmol, 1.0 eq) was added 30% methylamine in ethanol (2.0 mL) at room temperature and the reaction mixture was stirred at 80° C. for 4 h. The resulting mixture was concentrated under reduced pressure to afford tert-butyl-N-[3-({[(3aR,4R,6R,6aS)-2,2-dimethyl-6-[4-(methylamino)pyrrolo[2,3-d]pyrimidin-7-yl]-tetrahydro-3aH-cyclopenta[d][1,3]dioxol-4-yl]methyl}(3-bromo-2H-pyrazol-4-yl)amino)propyl]carbamate 218c (60 mg, crude) which was used directly in the next step without further purification. LCMS [conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-2 min (+), 1.50 L/min, ES, m/z]: T_{R}=1.30 min; [M+H]⁺: 619, 621.

### (1S,2R,3R,5R)-3-{[(3-Aminopropyl)(3-bromo-2H-pyrazol-4-yl)amino]methyl}-5-[4-(methylamino)pyrrolo[2,3-d]pyrimidin-7-yl]cyclopentane-1,2-diol (606)

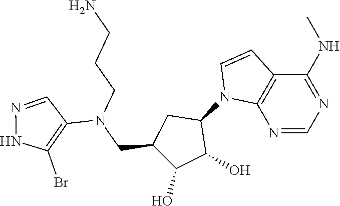 [SMILES: CNc1ncnc2c1ccn2[C@@H]1C[C@H](CN(CCCN)c2cn[nH]c2Br)[C@@H](O)[C@H]1O]

To a solution of tert-butyl-N-[3-({[(3aR,4R,6R,6aS)-2,2-dimethyl-6-[4-(methylamino)pyrrolo[2,3-d]pyrimidin-7-yl]-tetrahydro-3aH-cyclopenta[d][1,3]dioxol-4-yl]methyl}(3-bromo-2H-pyrazol-4-yl)amino)propyl]carbamate 218c (50 mg, 0.08 mmol, 1.0 eq) in DCM (1.0 mL), at room temperature, was added TFA (1.0 mL) and the reaction mixture was stirred at 50° C. for 1 h. The resulting mixture was concentrated under reduced pressure and the crude residue was dissolved in MeOH (3.0 mL) and adjusted to pH 8 with NH₃·H₂O. The crude sample was purified by Prep-HPLC [Column: YMC-Actus Triart C18, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 15% B to 40% B in 10 min, 40% B; Wave Length: 254 nm]: to afford (1S,2R,3R,5R)-3-{[(3-aminopropyl)(3-bromo-2H-pyrazol-4-yl)amino]methyl}-5-[4-(methylamino)pyrrolo[2,3-d]pyrimidin-7-yl]cyclopentane-1,2-diol 606 (7.4 mg, 19%) as a white solid. LCMS [conditionsZORBAX SB-Aq, 50*4.6 mm, 1.8 μm, Mobile Phase A: Water/0.02% TFA, Mobile Phase B: Acetonitrile, 5%-95% B-3 min (+), 1.50 L/min, ES, m/z]: T_{R}=1.14 min; [M+H]⁺: 479, 481.1H NMR (400 MHZ, DMSO-d₆) δ 8.07 (s, 1H), 7.58 (s, 1H), 7.20 (d, J=3.6 Hz, 1H), 6.52 (d, J=3.5 Hz, 1H), 4.85-4.72 (m, 1H), 4.22-4.14 (m, 1H), 3.81-3.76 (m, 1H), 3.14-3.04 (m, 1H), 2.97-2.77 (m, 6H), 2.55 (t, J=7.1 Hz, 2H), 2.22-2.06 (m, 1H), 2.06-1.94 (m, 1H), 1.55-1.39 (m, 3H).

### Synthesis of (1R,2S,3R,5R)-3-(4-amino-5-(4-phenoxythiazol-2-yl)-7H-pyrrolo[2,3-d]pyrimidin-7-yl)-5-(((3-(phenethylamino)propyl)amino)methyl)cyclopentane-1,2-diol (607)

### tert-Butyl-(3-((((3aR,4R,6R,6aS)-6-(4-amino-5-(4-phenoxythiazol-2-yl)-7H-pyrrolo[2,3-d]pyrimidin-7-yl)-2,2-dimethyltetrahydro-4H-cyclopenta[d][1,3]dioxol-4-yl)methyl)(tert-butoxycarbonyl)amino)propyl)(phenethyl)carbamate (209r)

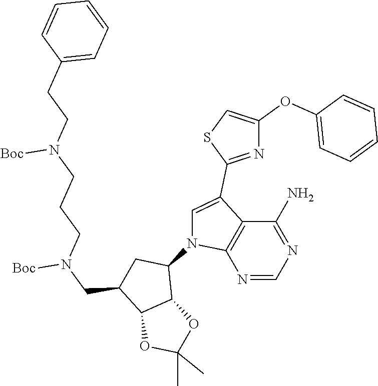 [SMILES: CC(C)(C)OC(=O)N(CCCN(C[C@H]1C[C@@H](n2cc(-c3nc(Oc4ccccc4)cs3)c3c(N)ncnc32)[C@@H]2OC(C)(C)O[C@H]12)C(=O)OC(C)(C)C)CCc1ccccc1]

To a solution of tert-butyl-N-[3-({[(3aR,4R,6R,6aS)-6-{4-amino-5-iodopyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 130c (90 mg, 0.12 mmol, 1.0 eq) and 4-phenoxy-2-(tributylstannyl)-1,3-thiazole 208c (159 mg, 0.30 mmol, 3.0 eq) in toluene (1.0 mL) was added Pd (PPh₃) 2Cl₂ (8.0 mg, 0.01 mmol, 0.1 eq) at room temperature and reaction mixture was stirred at 100° C. for 30 min, under N₂. The resulting mixture was extracted with AcOEt (3×10 mL) and the combined organic layers were washed with brine, dried over anhydrous Na₂SO₄ and the filtrate was concentrated under reduced pressure. The crude residue was purified by silica gel column chromatography (1:1 AcOEt: Petroleum ether) to afford tert-butyl-N-[3-({[(3aR,4R,6R,6aS)-6-[4-amino-5-(4-phenoxy-1,3-thiazol-2-yl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate 209c (88 mg, 84%) as a yellow oil. LCMS [conditions: L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-2.5 min-1.2 mL-P.lcm, 1.20 mL/min]: T_{R}=2.23 min; [M+H]⁺: 840.

### (1R,2S,3R,5R)-3-(4-Amino-5-(4-phenoxythiazol-2-yl)-7H-pyrrolo[2,3-d]pyrimidin-7-yl)-5-(((3-(phenethylamino)propyl)amino)methyl)cyclopentane-1,7-diol (607)

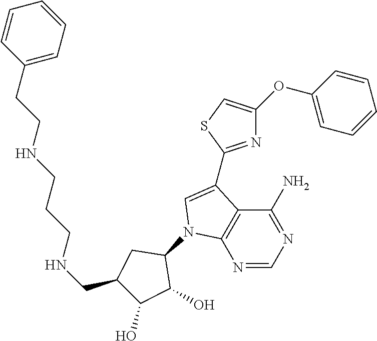 [SMILES: Nc1ncnc2c1c(-c1nc(Oc3ccccc3)cs1)cn2[C@@H]1C[C@H](CNCCCNCCc2ccccc2)[C@@H](O)[C@H]1O]

To a solution of tert-butyl-N-[3-({[(3aR,4R,6R,6aS)-6-[4-amino-5-(4-phenoxy-1,3-thiazol-2-yl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate 209c (78 mg, 0.09 mmol, 1.0 eq) in DCM (1.0 mL) was added TFA (1.0 mL) at room temperature and the reaction mixture was stirred at 50° C. for 1 h. The resulting mixture was concentrated under reduced pressure and the sample was adjusted to pH 8 with NH₃·H₂O. The crude residue was purified by Prep-HPLC [Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 25% B to 55% B in 7 min, 55% B; Wave Length: 220 nm]: to afford (1R,2S,3R,5R)-3-[4-amino-5-(4-phenoxy-1,3-thiazol-2-yl)pyrrolo[2,3-d]pyrimidin-7-yl]-5-[({3-[(2-phenylethyl)amino]propyl}amino)methyl]cyclopentane-1,2-diol 607 (36.1 mg, 64%) as a yellow solid. LCMS [conditions: ZORBAX SB-Aq, 50*4.6 mm, 1.8 μm, Mobile Phase A: Water/0.02% TFA, Mobile Phase B: Acetonitrile, 5%-95% B-3 min-1.5.lcm, 1.50 mL/min]: T_{R}=1.20 min; [M+H]⁺: 600. 1H NMR (300 MHZ, DMSO-d₆) δ 8.62 (s, 1H), 8.40 (s, 1H), 7.53-7.40 (m, 2H), 7.40-7.31 (m, 2H), 7.31-7.20 (m, 4H), 7.25-7.13 (m, 2H), 6.88 (s, 1H), 5.08-4.94 (m, 1H), 4.34-4.24 (m, 1H), 4.01-3.91 (m, 1H), 3.28-3.01 (m, 8H), 2.98-2.84 (m, 2H), 2.46-2.29 (m, 2H), 2.16-2.02 (m, 2H), 1.82-1.69 (m, 1H).

### Synthesis of 4-phenoxy-2-(tributylstannyl) thiazole (208c)

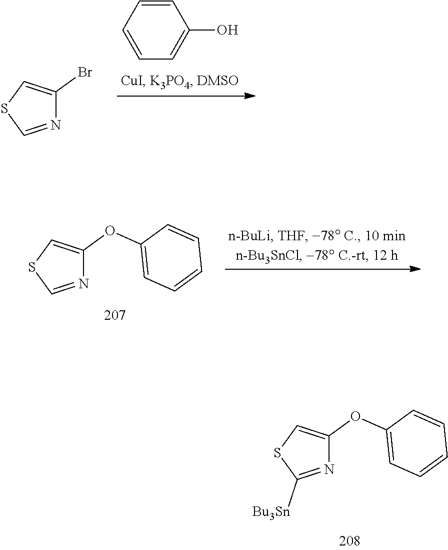

### 4-Phenoxythiazole (207c) [WO20202167791]

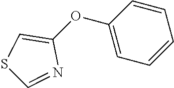 [SMILES: c1ccc(Oc2cscn2)cc1]

To phenol (860 mg, 9.20 mmol, 1.5 eq) in DMSO (10 mL) were added 4-bromo-1,3-thiazole (1.00 g, 6.10 mmol, 1.0 eq), Cul (106 mg, 0.60 mmol, 0.1 eq), picolinic acid (150 mg, 1.20 mmol, 0.2 eq), and K₃PO₄ (3.80 g, 18.1 mmol, 3.0 eq) at room temperature and the reaction mixture was heated under microwave radiation at 150° C. for 1 h. The resulting mixture was allowed to cool and was then extracted with AcOEt (3×30 mL). The combined organic layers were washed with brine, dried over anhydrous Na₂SO₄ and the filtrate was concentrated under reduced pressure. The crude residue was purified by reverse flash chromatography [column, C18 silica gel; mobile phase, MeOH in TFA water, 20% to 90% gradient over 15 min; detector UV 254 nm]: to afford 4-phenoxy-1,3-thiazole 207c (550 mg, 51%) as a yellow oil. LCMS [conditions: XBridge C18, 50*3.0 mm, 3.5 μm, Mobile Phase A: Water/5 mM NH₄HCO₃, Mobile Phase B: Methanol, 10%-95% B-2 min-1.5.lcm, 1.50 mL/min]: T_{R}=1.31 min; [M+H]⁺: 178. ¹H NMR (400 MHZ, Methanol-d₄) δ 8.86 (d, J=2.4 Hz, 1H), 7.44-7.34 (m, 2H), 7.22-7.13 (m, 1H), 7.12-7.04 (m, 2H), 6.69 (d, J=2.4 Hz, 1H).

### 4-Phenoxy-2-(tributylstannyl) thiazole (208c)

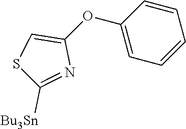

To a stirred solution of 4-phenoxy-1,3-thiazole 207c (500 mg, 2.80 mmol, 1.0 eq) in THF (5.0 mL) was added n-BuLi (2.4 M in hexanes, 1.3 mL, 3.10 mmol, 1.1 eq) dropwise, at −78° C., under N₂ and the resulting mixture was stirred at −78° C. for 10 min. To the above mixture was added tributylchlorostannane (1.80 g, 5.60 mmol, 2.0 eq) dropwise, at −78° C., and the reaction mixture was stirred at room temperature for 12 h. The resulting mixture was extracted with AcOEt (3×20 mL) and the combined organic layers were washed with brine, dried over anhydrous Na₂SO₄ and the filtrate was concentrated under reduced pressure to afford 4-phenoxy-2-(tributylstannyl)-1,3-thiazole 208c (1.1 g, crude) was used in the next step directly without further purification. LCMS [conditions: Kinetex XB-C18, 3.0*30 mm, 2.0 μm, Mobile Phase A: Water/0.1% FA, Mobile Phase B: Acetonitrile/0.05% FA, 5%-95% B-3 min (+).1.5.lcm, 1.50 mL/min]: T_{R}=2.85 min; [M+H]⁺: 468.

### Synthesis of (1R,2S,3R,5R)-3-{5-ethenylpyrrolo[2,3-d]pyrimidin-7-yl}-5-[{{3-[(2-phenylethyl)amino]propyl}amino)methyl]cyclopentane-1,2-diol (608)

### tert-Butyl-N-[3-({[(3aR,4R,6R,6aS)-2,2-dimethyl-6-{pyrrolo[2,3-d]pyrimidin-7-yl}-tetrahydro-3aH-cyclopenta[d][1,3]dioxol-4-yl]methyl}(tert-butoxycarbonyl)amino)propyl]-N-(2-phenylethyl)carbamate (219c)

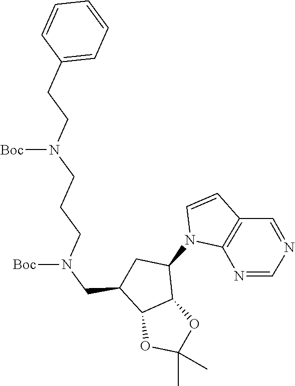 [SMILES: CC(C)(C)OC(=O)N(CCCN(C[C@H]1C[C@@H](n2ccc3cncnc32)[C@@H]2OC(C)(C)O[C@H]12)C(=O)OC(C)(C)C)CCc1ccccc1]

To a solution of tert-butyl-N-[3-({[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 107c (0.50 g, 0.70 mmol, 1.0 eq) in MeOH (10 mL) was added 10% Pd/C (50 mg) and the reaction mixture was stirred for 30 min at room temperature, under H₂. The resulting mixture was filtered, the filter cake was washed with MeOH and the combined filtrate was concentrated under reduced pressure to afford tert-butyl-N-[3-({[(3aR,4R,6R,6aS)-2,2-dimethyl-6-{pyrrolo[2,3-d]pyrimidin-7-yl}-tetrahydro-3aH-cyclopenta[d][1,3]dioxol-4-yl]methyl}(tert-butoxycarbonyl)amino)propyl]-N-(2-phenylethyl)carbamate 219c (400 mg, 84%) as a pale yellow solid. LCMS [conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min (+), 1.50 L/min, ES, m/z]: T_{R}=2.46 min; [M+H]⁺: 650.1H NMR (300 MHZ, DMSO-d₆) δ 9.34 (s, 1H), 9.11 (s, 1H), 8.19 (s, 1H), 7.35-7.24 (m, 2H), 7.23-7.15 (m, 3H), 7.07-6.97 (m, 1H), 5.24-5.10 (m, 1H), 5.01-4.91 (m, 1H), 4.63-4.52 (m, 1H), 3.53-3.39 (m, 1H), 3.38-3.29 (m, 2H), 3.28-3.15 (m, 1H), 3.13 (s, 4H), 3.12-3.05 (m, 1H), 2.82-2.71 (m, 2H), 2.30-2.24 (m, 1H), 2.28-2.10 (m, 1H), 1.74-1.63 (m, 2H), 1.46 (s, 3H), 1.37 (s, 18H), 1.22 (s, 3H).

### tert-Butyl-N-[3-({[(3aR,4R,6R,6aS)-6-{5-bromopyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate (220c)

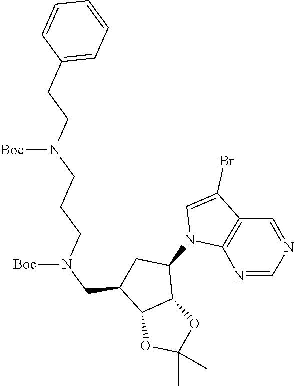 [SMILES: CC(C)(C)OC(=O)N(CCCN(C[C@H]1C[C@@H](n2cc(Br)c3cncnc32)[C@@H]2OC(C)(C)O[C@H]12)C(=O)OC(C)(C)C)CCc1ccccc1]

To tert-butyl-N-[3-({[(3aR,4R,6R,6aS)-2,2-dimethyl-6-{pyrrolo[2,3-d]pyrimidin-7-yl}-tetrahydro-3aH-cyclopenta[d][1,3]dioxol-4-yl]methyl}(tert-butoxycarbonyl)amino)propyl]-N-(2-phenylethyl)carbamate 219c (0.30 g, 0.50 mmol, 1.0 eq) in DMF (2.0 mL) was added NBS (0.09 g, 0.50 mmol, 1.1 eq) in DMF (1.0 mL), dropwise over 2 min, at 0° C. and the reaction mixture was stirred at 0° C. for 20 min. The reaction was quenched by the addition of saturated aq. NaS2O₃ at 0° C. and the resulting mixture was extracted with AcOEt (3×3 mL). The combined organic layers were washed with brine, dried over anhydrous Na₂SO₄ and the filtrate was concentrated under reduced pressure. The crude residue was purified by silica gel column chromatography (80:20 Petroleum ether: AcOEt) to afford tert-butyl-N-[3-({[(3aR,4R,6R,6aS)-6-{5-bromopyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 220c (300 mg, 89%) as a pale yellow oil. LCMS [conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min (+), 1.50 L/min, ES, m/z]: T_{R}=2.63 min; [M+H]⁺: 728,730.1H NMR (300 MHZ, DMSO-d₆) δ 8.94 (s, 1H), 8.89 (s, 1H), 8.16 (s, 1H), 7.34-7.24 (m, 2H), 7.24-7.15 (m, 3H), 5.18-5.04 (m, 1H), 4.98-4.88 (m, 1H), 4.60-4.49 (m, 1H), 3.52-3.33 (m, 2H), 3.26-3.14 (m, 1H), 3.11 (d, J=8.3 Hz, 4H), 2.82-2.73 (m, 2H), 2.41-2.35 (m, 1H), 2.27-2.19 (m, 2H), 2.17-2.06 (m, 1H), 1.71-1.65 (m, 2H), 1.46 (s, 3H), 1.37 (s, 18H), 1.19 (s, 3H)

### (1R,2S,3R,5R)-3-{5-Bromopyrrolo[2,3-d]pyrimidin-7-yl}-5-[{{3-[(2-phenylethyl)amino]propyl}amino)methyl]cyclopentane-1,2-diol (221c)

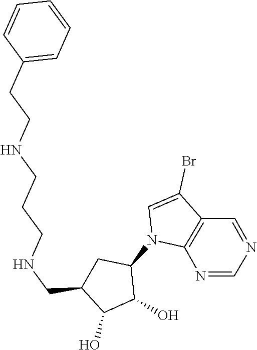 [SMILES: O[C@@H]1[C@@H](CNCCCNCCc2ccccc2)C[C@@H](n2cc(Br)c3cncnc32)[C@@H]1O]

To a solution of tert-butyl-N-[3-({[(3aR,4R,6R,6aS)-6-{5-bromopyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 220c (0.30 g, 0.40 mmol, 1.0 eq) in DCM (2.0 mL) was added TFA (3.0 mL) and the reaction mixture was stirred at 50° C. for 2 h. The resulting mixture was concentrated under reduced pressure to afford (1R,2S,3R,5R)-3-{5-bromopyrrolo[2,3-d]pyrimidin-7-yl}-5-[{{3-[(2-phenylethyl)amino]propyl}amino)methyl]cyclopentane-1,2-diol 221c (200 mg, crude) which was used in the next step directly without further purification. LCMS [conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-2 min (+), 1.50 L/min, ES, m/z]: T_{R}=1.15 min; [M+H]⁺: 488, 490.

### (1R,2S,3R,5R)-3-{5-Ethenylpyrrolo[2,3-d]pyrimidin-7-yl}-5-[{{3-[(2-phenylethyl)amino]propyl}amino)methyl]cyclopentane-12-diol (608)

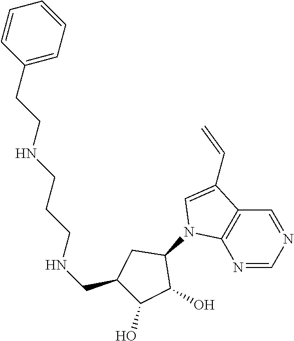 [SMILES: C=Cc1cn([C@@H]2C[C@H](CNCCCNCCc3ccccc3)[C@@H](O)[C@H]2O)c2ncncc12]

To (1R,2S,3R,5R)-3-{5-bromopyrrolo[2,3-d]pyrimidin-7-yl}-5-[({3-[(2-phenylethyl)amino]propyl}amino)methyl]cyclopentane-1,2-diol 221c (0.20 g, 0.40 mmol, 1.0 eq) in H₂O (0.2 mL) and dioxane (2.0 mL) were added 2-ethenyl-4,4,5,5-tetramethyl-1,3,2-dioxaborolane (0.31 g, 2.00 mmol, 5.0 eq), Pd (dtbpf) Cl₂ (3.0 mg, 0.004 mmol, 0.01 eq) and K₃PO₄ (0.43 g, 2.00 mmol, 5.0 eq) at room temperature under N₂ and the reaction mixture was stirred at 80° C. for 30 min. The resulting mixture was filtered and the filtrate was purified by HPLC [Column: Welch Ultimate XB-C18, 50*250 mm, 10 μm; Mobile Phase A: Water (10 mmol/L NH₄HCO₃+0.1% NH₃·H₂O), Mobile Phase B: ACN; Flow rate: 30 mL/min; Gradient: 20% B to 50% B in 10 min, Wave Length: 254 nm]: to afford (1R,2S,3R,5R)-3-{5-ethenylpyrrolo[2,3-d]pyrimidin-7-yl}-5-[({3-[(2-phenylethyl)amino]propyl}amino)methyl]cyclopentane-1,2-diol 608 (100 mg, 56%) as an off-white solid. LCMS [conditions ZORBAX SB-Aq, 50*4.6 mm, 1.8 μm, Mobile Phase A: Water/0.02% TFA, Mobile Phase B: Acetonitrile, 5%-95% B-3 min, 1.50 L/min, ES, m/z]: T_{R}=1.03 min; [M+H]⁺: 438. ¹H NMR (300 MHZ, DMSO-d₆+D₂O) δ 9.30 (s, 1H), 8.81 (s, 1H), 7.94 (s, 1H), 7.32-7.10 (m, 5H), 6.85 (dd, J=17.9, 11.2 Hz, 1H), 5.82 (dd, J=17.9, 1.2 Hz, 1H), 5.23 (dd, J=11.2, 1.2 Hz, 1H), 5.01 (q, J=9.0 Hz, 1H), 4.85 (br s, 1H), 4.29-4.18 (m, 1H), 3.84-3.78 (m, 1H), 2.96-2.53 (m, 10H), 2.28-2.15 (m, 1H), 2.14-2.01 (m, 1H), 1.63-1.46 (m, 3H).

### Synthesis of (1R,2S,3R,5R)-3-{5-ethylpyrrolo[2,3-d]pyrimidin-7-yl}-5-[({3-[(2-phenylethyl)amino]propyl}amino)methyl]cyclopentane-1,2-diol (609)

### (1R,2S,3R,5R)-3-{5-Ethylpyrrolo[2,3-d]pyrimidin-7-yl}-5-[{{3-[(2-phenylethyl)amino]propyl}amino)methyl]cyclopentane-1,2-diol (609)

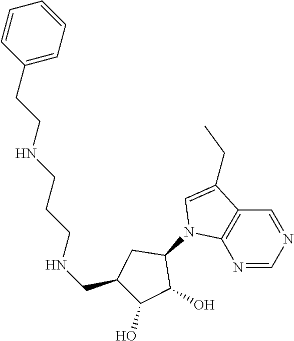 [SMILES: CCc1cn([C@@H]2C[C@H](CNCCCNCCc3ccccc3)[C@@H](O)[C@H]2O)c2ncncc12]

To a solution of (1R,2S,3R,5R)-3-{5-ethenylpyrrolo[2,3-d]pyrimidin-7-yl}-5-[({3-[(2-phenylethyl)amino]propyl}amino)methyl]cyclopentane-1,2-diol 608 (80 mg, 0.20 mmol, 1 eq) in MeOH (2.0 mL) was added 10% Pd/C (19.5 mg, 0.20 mmol, 1.0 eq) and the reaction mixture was stirred for 30 min at room temperature under H₂. The resulting mixture was filtered, the filter cake was washed with MeOH and the combined filtrate was evaporated. The crude residue was purified by Prep-HPLC [Column: Welch Ultimate XB-C18, 50*250 mm, 10 μm; Mobile Phase A: 0.1% HCl, Mobile Phase B: ACN; Flow rate: 90 mL/min; Gradient: 5% B-5% B-3 min, 5% B-20% B-12mim]: to afford (1R,2S,3R,5R)-3-{5-ethylpyrrolo[2,3-d]pyrimidin-7-yl}-5-[({3-[(2-phenylethyl)amino]propyl}amino)methyl]cyclopentane-1,2-diol 609 (11.5 mg, 14%) as a colorless oil. LCMS [conditions ZORBAX SB-Aq, 50*4.6 mm, 1.8 μm, Mobile Phase A: Water/0.02% TFA, Mobile Phase B: Acetonitrile, 5%-95% B-3 min (+), 1.50 L/min, ES, m/z]: T_{R}=0.97 min; [M+H]⁺: 438. ¹H NMR (300 MHZ, DMSO-d₆) δ 9.37 (s, 1H), 9.26-8.99 (m, 4H), 7.97 (s, 1H), 7.45-7.20 (m, 5H), 5.14-4.99 (m, 1H), 4.30-4.19 (m, 1H), 4.04-3.92 (m, 1H), 3.16-2.91 (m, 10H), 2.83 (q, J=7.4 Hz, 2H), 2.42-2.31 (m, 2H), 2.19-2.03 (m, 2H), 1.80-1.67 (m, 1H), 1.32 (t, J=7.5 Hz, 3H).

### Synthesis of (1R,2S,3R,5R)-3-(4-methyl-5-vinyl-7H-pyrrolo[2,3-d]pyrimidin-7-yl)-5-(((3-(phenethylamino)propyl)amino)methyl)cyclopentane-1,2-diol (610)

### tert-Butyl-(3-((tert-butoxycarbonyl)(phenethyl)amino)propyl)(((3aR,4R,6R,6aS)-2,2-dimethyl-6-(4-methyl-7H-pyrrolo[2,3-d]pyrimidin-7-yl)tetrahydro-4H-cyclopenta[d][1,3]dioxol-4-yl)methyl)carbamate (222c)

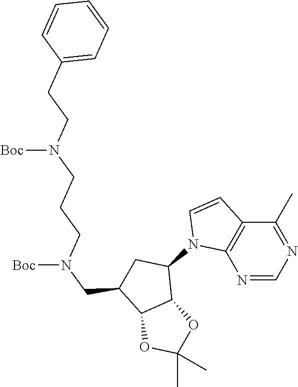 [SMILES: Cc1ncnc2c1ccn2[C@@H]1C[C@H](CN(CCCN(CCc2ccccc2)C(=O)OC(C)(C)C)C(=O)OC(C)(C)C)[C@H]2OC(C)(C)O[C@H]21]

To a solution of tert-butyl-N-[3-({[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 107c (0.50 g, 0.70 mmol, 1.0 eq) in THF (5.0 mL) was added, Pd (PPh₃) 2Cl₂ (25.6 mg, 0.04 mmol, 0.05 eq) and, 1.0 M Zn (CH₃) 2 in toluene (2.4 mL, 2.40 mmol, 3.0 eq) at room temperature and the reaction mixture was stirred at 80° C. under N₂ for 4 h. The reaction was quenched with NH₄Cl (saturated solution) at 0° C. and the resulting mixture was extracted with ethyl acetate (3×30 mL). The combined organic layers were washed with brine, dried over anhydrous Na₂SO₄ and the filtrate was concentrated under reduced pressure. The crude residue was purified by silica gel column chromatography (3:7 ethyl acetate:petroleum ether) to afford tert-butyl-N-[3-({[(3aR,4R,6R,6aS)-2,2-dimethyl-6-{4-methylpyrrolo[2,3-d]pyrimidin-7-yl}-tetrahydro-3aH-cyclopenta[d][1,3]dioxol-4-yl]methyl}(tert-butoxycarbonyl)amino)propyl]-N-(2-phenylethyl)carbamate 222c (0.42 g, 87%) as a colorless oil. LCMS [conditions: ZORBAX SB-Aq, 50*4.6 mm, 1.8 μm, Mobile Phase A: Water/0.02% TFA, Mobile Phase B: Acetonitrile, 2%-100% B-2 min (+).lcm, 1.20 mL/min]: T_{R}=1.35 min; [M+H]⁺: 664.

### tert-Butyl-(((3aR,4R,6R,6aS)-6-(5-bromo-4-methyl-7H-pyrrolo[2,3-d]pyrimidin-7-yl)-2,2-dimethyltetrahydro-4H-cyclopenta[d][1,3]dioxol-4-yl)methyl)(3-((tert-butoxycarbonyl)(phenethyl)amino)propyl)carbamate (223c)

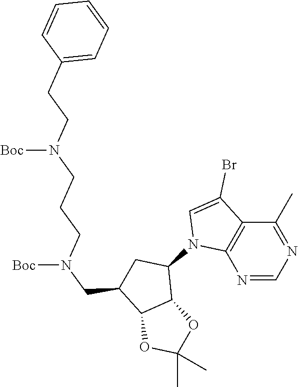 [SMILES: Cc1ncnc2c1c(Br)cn2[C@@H]1C[C@H](CN(CCCN(CCc2ccccc2)C(=O)OC(C)(C)C)C(=O)OC(C)(C)C)[C@H]2OC(C)(C)O[C@H]21]

To tert-butyl-N-[3-({[(3aR,4R,6R,6aS)-2,2-dimethyl-6-{4-methylpyrrolo[2,3-d]pyrimidin-7-yl}-tetrahydro-3aH-cyclopenta[d][1,3]dioxol-4-yl]methyl}(tert-butoxycarbonyl)amino)propyl]-N-(2-phenylethyl)carbamate 222c (0.40 g, 0.60 mmol, 1.0 eq) in DMF (5.0 mL) was added a solution of NBS (0.13 g, 0.7 mmol, 1.2 eq) in DMF (1.0 mL), dropwise, at 0° C. and the reaction mixture was stirred at room temperature for 1 h. The reaction was quenched by the addition of sodium thiosulfate solution at 0° C. and the resulting mixture was extracted with AcOEt (3×30 mL). The combined organic layers were washed with brine and dried over anhydrous Na₂SO₄. The filtrate was concentrated under reduced pressure and the crude residue was purified by silica gel column chromatography (2:3 ethyl acetate:petroleum ether) to afford tert-butyl-N-[3-({[(3aR,4R,6R,6aS)-6-{5-bromo-4-methylpyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 223c (0.37 mg, 82%) as a colorless oil. LCMS [conditions: XBridge C18, 50*3.0 mm, 3.5 μm, Mobile Phase A: Water/5 mM NH₄HCO₃, Mobile Phase B: Methanol, 10%-95% B-2 min (+).Icm, 1.20 mL/min]: T_{R}=1.62 min; [M+H]⁺: 742, 744.

### (1R,2S,3R,5R)-3-(5-Bromo-4-methyl-7H-pyrrolo[2,3-d]pyrimidin-7-yl)-5-(((3-(phenethylamino)propyl)amino)methyl)cyclopentane-1,2-diol (224c)

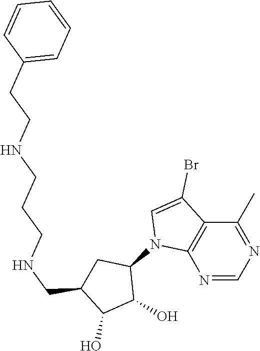 [SMILES: Cc1ncnc2c1c(Br)cn2[C@@H]1C[C@H](CNCCCNCCc2ccccc2)[C@@H](O)[C@H]1O]

To tert-butyl-N-[3-({[(3aR,4R,6R,6aS)-6-{5-bromo-4-methylpyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 223c (0.35 g, 0.47 mmol, 1.0 eq) was added TFA (3.0 mL) and the reaction mixture was stirred at 50° C. for 0.5 h. The resulting mixture was concentrated under reduced pressure to afford (1R,2S,3R,5R)-3-{5-bromo-4-methylpyrrolo[2,3-d]pyrimidin-7-yl}-5-[({3-[(2-phenylethyl)amino]propyl}amino)methyl]cyclopentane-1,2-diol 224c (0.20 g, crude) which was used in the next step directly without purification. LCMS [conditions: ZORBAX SB-Aq, 50*4.6 mm, 1.8 μm, Mobile Phase A: Water/0.02% TFA, Mobile Phase B: Acetonitrile, 2%-100% B-2 min (+).lcm, 1.20 mL/min)]: T_{R}=0.65 min; [M+H]⁺: 502, 504.

### (1R,2S,3R,5R)-3-(4-Methyl-5-vinyl-7H-pyrrolo[2,3-d]pyrimidin-7-yl)-5-(((3-(phenethylamino)propyl)amino)methyl)cyclopentane-1,2-diol (610)

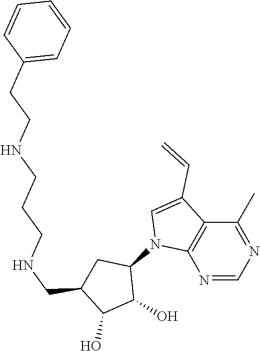 [SMILES: C=Cc1cn([C@@H]2C[C@H](CNCCCNCCc3ccccc3)[C@@H](O)[C@H]2O)c2ncnc(C)c12]

To a stirred solution of (1R,2S,3R,5R)-3-{5-bromo-4-methylpyrrolo[2,3-d]pyrimidin-7-yl}-5-[{{3-[(2-phenylethyl)amino]propyl}amino)methyl]cyclopentane-1,2-diol 224c (0.20 g, 0.40 mmol, 1 eq) and 2-ethenyl-4,4,5,5-tetramethyl-1,3,2-dioxaborolane (92 mg, 0.60 mmol, 1.5 eq) in 1,4-dioxane (1.0 mL) and H₂O (0.1 mL) were added Pd (DtBPF) Cl₂ (13.0 mg, 0.02 mmol, 0.05 eq) and K₃PO₄ (0.25 g, 1.20 mmol, 3.0 eq) and the reaction mixture was stirred at 80° C., under N₂, for 0.5 h. The resulting mixture was concentrated under reduced pressure, dissolved in methanol (3.0 mL) and the crude residue was purified by Prep-HPLC [Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 25% B to 65% B in 7 min, 65% B; Wave Length: 220 nm]: to afford (1R,2S,3R,5R)-3-{5-ethenyl-4-methylpyrrolo[2,3-d]pyrimidin-7-yl}-5-[{{3-[(2-phenylethyl)amino]propyl}amino)methyl]cyclopentane-1,2-diol 610 (90 mg, 50%) as an off-white solid. LCMS [conditions ZORBAX SB-Aq, 50*4.6 mm, 1.8 μm, Mobile Phase A: Water/0.02% TFA, Mobile Phase B: Acetonitrile, 5%-95% B-3 min-1.5.lcm, 1.50 mL/min, ES, m/z]: T_{R}=0.99 min; [M+H]⁺: 450.1H NMR (400 MHZ, DMSO-d₆+D₂O) δ 8.57 (s, 1H), 7.96 (s, 1H), 7.28-7.20 (m, 2H), 7.20-7.11 (m, 3H), 7.09-6.97 (m, 1H), 5.67 (d, J=17.6 Hz, 1H), 5.21 (d, J=12.0 Hz, 1H), 5.06-4.95 (m, 1H), 4.29-4.21 (m, 1H), 3.84-3.79 (m, 1H), 3.37-2.87 (m, 2H), 2.79-2.51 (m, 11H), 2.26-2.08 (m, 2H), 1.62-1.49 (m, 3H).

### Synthesis of (1R,2S,3R,5R)-3-(5-ethyl-4-methyl-7H-pyrrolo[2,3-d]pyrimidin-7-yl)-5-(((3-(phenethylamino)propyl)amino)methyl)cyclopentane-1,2-diol (611)

### (1R,2S,3R,5R)-3-(5-Ethyl-4-methyl-7H-pyrrolo[2,3-d]pyrimidin-7-yl)-5-(((3-(phenethylamino)propyl)amino)methyl)cyclopentane-17-diol (611)

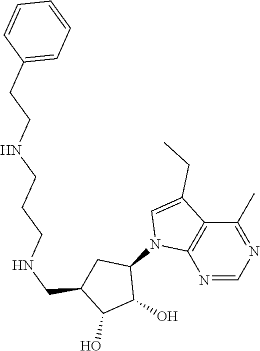 [SMILES: CCc1cn([C@@H]2C[C@H](CNCCCNCCc3ccccc3)[C@@H](O)[C@H]2O)c2ncnc(C)c12]

To a solution of (1R,2S,3R,5R)-3-{5-ethenyl-4-methylpyrrolo[2,3-d]pyrimidin-7-yl}-5-[({3-[(2-phenylethyl)amino]propyl}amino)methyl]cyclopentane-1,2-diol 610 (45.0 mg, 0.1 mmol, 1.0 eq) in THF (1.0 mL) was added 10% Pd/C (10.7 mg, 0.01 mmol, 0.1 eq) and the reaction mixture was hydrogenated at room temperature, under 30 psi of hydrogen, in a pressure tank for 0.5 h. The resulting mixture was filtered through a Celite pad and concentrated under reduced pressure. The crude residue was purified by Prep-HPLC [Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 20% B to 80% B in 7 min, 65% B; Wave Length: 220 nm]: to afford (1R,2S,3R,5R)-3-{5-ethyl-4-methylpyrrolo[2,3-d]pyrimidin-7-yl}-5-[{{3-[(2-phenylethyl)amino]propyl}amino)methyl]cyclopentane-1,2-diol 611 (16.0 mg, 35%) as a white solid. LCMS [conditions ZORBAX SB-Aq, 50*4.6 mm, 1.8 μm, Mobile Phase A: Water/0.02% TFA, Mobile Phase B: Acetonitrile, 5%-95% B-5 min-1.0-AB.lcm, 1.50 mL/min, ES, m/z]: T_{R}=1.86 min; [M+H]⁺: 452. ¹H NMR (400 MHZ, DMSO-d₆+D₂O) δ 8.52 (s, 1H), 7.41 (s, 1H), 7.29-7.22 (m, 2H), 7.20-7.12 (m, 3H), 5.01-4.88 (m, 1H), 4.25-4.12 (m, 1H), 3.88-3.74 (m, 1H), 2.84 (q, J=7.5 Hz, 2H), 2.79-2.52 (m, 13H), 2.25-1.97 (m, 2H), 1.66-1.46 (m, 3H), 1.26 (t, J=7.4 Hz, 3H).

### Synthesis of (1R,2S,3R,5R)-3-{4-chloro-5-ethenylpyrrolo[2,3-d]pyrimidin-7-yl}-5-[{{3-[(2-phenylethyl)amino]propyl}amino)methyl]cyclopentane-1,2-diol (612)

### (1R,2S,3R,5R)-3-(4-Chloro-5-iodo-7H-pyrrolo[2,3-d]pyrimidin-7-yl)-5-(((3-(phenethylamino)propyl)amino)methyl)cyclopentane-17-diol (225c)

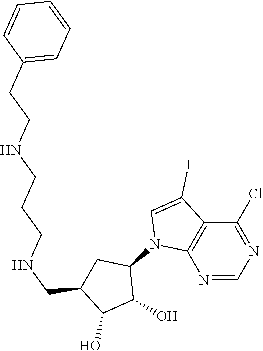 [SMILES: O[C@@H]1[C@@H](CNCCCNCCc2ccccc2)C[C@@H](n2cc(I)c3c(Cl)ncnc32)[C@@H]1O]

To tert-butyl-N-[3-({[(3aR,4R,6R,6aS)-6-{4-chloro-5-iodopyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 129c (70 mg, 0.09 mmol, 1.0 eq) was added TFA (2.0 mL) at room temperature and the resulting mixture was stirred at 50° C. for 1 h. The resulting mixture was concentrated under reduced pressure to afford (1R,2S,3R,5R)-3-(4-chloro-5-iodo-7H-pyrrolo[2,3-d]pyrimidin-7-yl)-5-(((3-(phenethylamino)propyl)amino)methyl)cyclopentane-1,2-diol 225c (40 mg, crude) which was used in the next step directly without further purification. LCMS [conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min (+), 1.50 L/min, ES, m/z]: T_{R}=1.55 min; [M+H]⁺: 570.

### (1R,2S,3R,5R)-3-{4-Chloro-5-ethenylpyrrolo[2,3-d]pyrimidin-7-yl}-5-[{{3-[(2-phenylethyl)amino]propyl}amino)methyl]cyclopentane-1,2-diol (61)

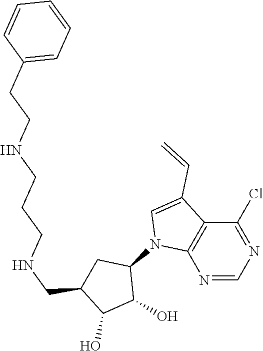 [SMILES: C=Cc1cn([C@@H]2C[C@H](CNCCCNCCc3ccccc3)[C@@H](O)[C@H]2O)c2ncnc(Cl)c12]

To a suspension of (1R,2S,3R,5R)-3-{4-chloro-5-iodopyrrolo[2,3-d]pyrimidin-7-yl}-5-[({3-[(2-phenylethyl)amino]propyl}amino)methyl]cyclopentane-1,2-diol 225c (40 mg, 0.07 mmol, 1.0 eq) and 2-ethenyl-4,4,5,5-tetramethyl-1,3,2-dioxaborolane (21.6 mg, 0.14 mmol, 2.0 eq) and K₃PO₄ (89.4 mg, 0.42 mmol, 6.0 eq) in dioxane (1.0 mL) and water H₂O (0.10 mL) was added Pd (DtBPF) Cl₂ (5.0 mg, 0.007 mmol, 0.1 eq) at room temperature under N₂ and the reaction mixture was stirred at 80° C. for 30 min. The resulting mixture was filtered, the filtrate was concentrated under reduced pressure and the crude residue was purified by Prep-HPLC [Column: Xbridge C18,19*150 mm, 5 μm; Mobile Phase A: Water (20 mmol/L NH₄HCO₃+0.05% NH₃·H₂O), Mobile Phase B: ACN; Flow rate: 30 mL/min; Gradient: 20% B to 45% B in 10 min; Wave Length: 254 nm]: to afford (1R,2S,3R,5R)-3-{4-chloro-5-ethenylpyrrolo[2,3-d]pyrimidin-7-yl}-5-[{{3-[(2-phenylethyl)amino]propyl}amino)methyl]cyclopentane-1,2-diol 612 (1.8 mg, 5%) as a pale yellow oil. LCMS [conditions ZORBAX SB-Aq, 50*4.6 mm, 1.8 μm, Mobile Phase A: Water/0.02% TFA, Mobile Phase B: Acetonitrile, 5%-95% B-3 min (+), 1.50 L/min, ES, m/z]: T_{R}=1.25 min; [M+H]⁺: 470, 472. ¹H NMR (400 MHZ, DMSO-d₆+D₂O) δ 8.58 (s, 1H), 8.23 (s, 1H), 7.29-7.08 (m, 6H), 5.77 (d, J=17.6 Hz, 1H), 5.27 (d, J=11.2 Hz, 1H), 5.06-4.96 (m, 1H), 4.29-4.22 (m, 1H), 3.83-3.75 (m, 1H), 2.77-2.63 (m, 5H), 2.62-2.53 (m, 5H), 2.27-2.13 (m, 1H), 2.11-1.98 (m, 1H), 1.62-1.50 (m, 3H).

### Synthesis of (1R,2S,3R,5R)-3-(5-(4-benzylthiazol-2-yl)-7H-pyrrolo[2,3-d]pyrimidin-7-yl)-5-(((3-(phenethylamino)propyl)amino)methyl)cyclopentane-1,2-diol (613)

### (4-Benzylthiazol-2-yl) zinc (II) chloride (227c)

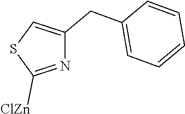

To 4-benzyl-2-bromo-1,3-thiazole (0.40 g, 1.57 mmol, 1.0 eq) in THF (2.0 mL) was added 2.5 M n-BuLi in hexane (0.7 mL, 1.73 mmol, 1.1 eq) at −78° C., and the resulting mixture was stirred at −78° C., under nitrogen, for 30 min. To this solution was then added 0.7 M zinc chloride in THF (3.4 mL, 2.36 mmol, 1.5 eq) at −78° C. and the resulting mixture was stirred at −40° C., under nitrogen for 30 min to afford a crude sample of (4-benzyl-1,3-thiazol-2-yl)(chloro) zinc) 227c crude (6.1 mL in THF) which was used in the next step directly without isolation.

### Tert-Butyl-(3-((tert-butoxycarbonyl)(phenethyl)amino)propyl)(((3aR,4R,6R,6aS)-6-(5-iodo-7H-pyrrolo[2,3-d]pyrimidin-7-yl)-2,2-dimethyltetrahydro-4H-cyclopenta[d][1,3]dioxol-4-yl)methyl)carbamate (226c)

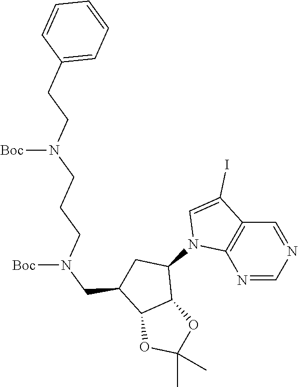 [SMILES: CC(C)(C)OC(=O)N(CCCN(C[C@H]1C[C@@H](n2cc(I)c3cncnc32)[C@@H]2OC(C)(C)O[C@H]12)C(=O)OC(C)(C)C)CCc1ccccc1]

To a stirred solution of tert-butyl-N-[3-({[(3aR,4R,6R,6aS)-2,2-dimethyl-6-{pyrrolo[2,3-d]pyrimidin-7-yl}-tetrahydro-3aH-cyclopenta[d][1,3]dioxol-4-yl]methyl}(tert-butoxycarbonyl)amino)propyl]-N-(2-phenylethyl)carbamate 219c (255 mg, 0.40 mmol, 1.0 eq) in DMF (4.0 mL) was added NIS (132 mg, 0.60 mmol, 1.5 eq), in several portions, at room temperature and the reaction mixture was stirred at room temperature, for 1 h, under nitrogen. The resulting mixture was concentrated under reduced pressure and the crude residue was purified by silica gel column chromatography (1:1 Petroleum ether: AcOEt) to afford tert-butyl-N-[3-({[(3aR,4R,6R,6aS)-6-{5-iodopyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 226c (180 mg, 59%) as a yellow solid. LCMS [column: Xbridge C18, 50*3.0 mm, 3.5 μm, Mobile Phase A: Water/5 mM NH₄HCO₃, Mobile Phase B: Methanol, 10%-95% B-3 min, 1.00 mL/min, ES, m/z]: T_{R}=2.44 min; [M+H]⁺: 776.

### Tert-Butyl-(((3aR,4R,6R,6aS)-6-(5-(4-benzylthiazol-2-yl)-7H-pyrrolo[2,3-d]pyrimidin-7-yl)-2,2-dimethyltetrahydro-4H-cyclopenta[d][1,3]dioxol-4-yl)methyl)(3-((tert-butoxycarbonyl)(phenethyl)amino)propyl)carbamate (228c)

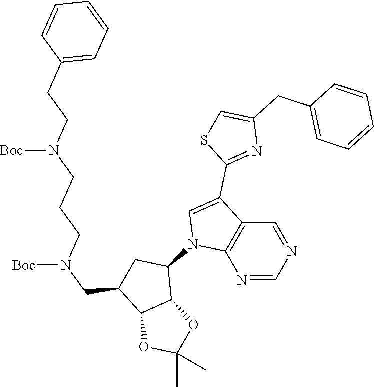 [SMILES: CC(C)(C)OC(=O)N(CCCN(C[C@H]1C[C@@H](n2cc(-c3nc(Cc4ccccc4)cs3)c3cncnc32)[C@@H]2OC(C)(C)O[C@H]12)C(=O)OC(C)(C)C)CCc1ccccc1]

To a suspension of tert-butyl-N-[3-({[(3aR,4R,6R,6aS)-6-{5-iodopyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 226c (105 mg, 0.13 mmol, 1.0 eq), Pd₂ (dba)₃ (24 mg, 0.03 mmol, 0.2 eq), tris(furan-2-yl)phosphane (12 mg, 0.05 mmol, 0.4 eq) and K₃PO₄ (86 mg, 0.40 mmol, 3.0 eq) in THF (2.0 mL) was added (4-benzyl-1,3-thiazol-2-yl)(chloro) zinc 227c crude (2.6 mL in THF, 0.65 mmol, 5.0 eq) at room temperature and the reaction mixture was stirred at 60° C. for 12 h, under nitrogen. The resulting mixture was diluted with EtOAc (20 mL), washed with H₂O (3×10 mL) and the organic layer was dried with anhydrous Na₂SO₄. The filtrate was concentrated under reduced pressure and the crude residue was purified by silica gel column chromatography (1:1 Petroleum ether: AcOEt) to afford tert-butyl-N-[3-({[(3aR,4R,6R,6aS)-6-[5-(4-benzyl-1,3-thiazol-2-yl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate 228c (45 mg, 40%) as a yellow solid. LCMS [column: L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min, 1.20 mL/min, ES, m/z]: T_{R}=2.70 min; [M+H]⁺: 823.

### (1R,2S,3R,5R)-3-(5-(4-Benzylthiazol-2-yl)-7H-pyrrolo[2,3-d]pyrimidin-7-yl)-5-(((3-(phenethylamino)propyl)amino)methyl)cyclopentane-1,7-diol (613)

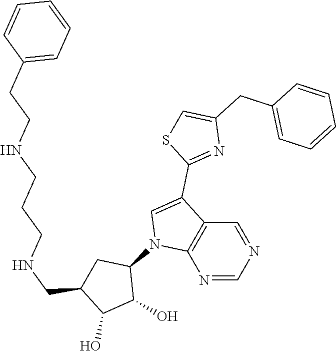 [SMILES: O[C@@H]1[C@@H](CNCCCNCCc2ccccc2)C[C@@H](n2cc(-c3nc(Cc4ccccc4)cs3)c3cncnc32)[C@@H]1O]

To tert-butyl-N-[3-({[(3aR,4R,6R,6aS)-6-[5-(4-benzyl-1,3-thiazol-2-yl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate 228c (45 mg, 0.06 mmol, 1.0 eq) was added 4M HCl in MeOH (2.0 mL) at room temperature and the reaction mixture was stirred at 50° C. for 1 h. The resulting mixture was concentrated under reduced pressure, adjusted to pH 10 with saturated (aq.) Na₂CO₃ and the crude residue was purified by Prep-HPLC [Column, Xbridge Shield RP18 OBD Column, 19*150 mm, 5 μm; mobile phase, Water (10 mmol/L NH₄HCO₃+0.1% NH₃·H₂O) and CAN (20% CAN up to 70% in 7.5 min); Detector, UV 254 nm]: to afford (1R,2S,3R,5R)-3-[5-(4-benzyl-1,3-thiazol-2-yl)pyrrolo[2,3-d]pyrimidin-7-yl]-5-[({3-[(2-phenylethyl)amino]propyl}amino)methyl]cyclopentane-1,2-diol 613 (15.7 mg, 49%) as an off-white solid. LCMS [column: Cortecs ZORBAX SB-Aq, 50*4.6 mm, 1.8 μm. Mobile phase A: Water/0.02% TFA, mobile phase B: Acetonitrile, 5%-95% B-3 min, 1.50 mL/min, ES, m/z]: T_{R}=1.33 min; [M+H]⁺: 583. ¹H NMR (400 MHZ, DMSO-d₆+D₂O) δ 9.41 (s, 1H), 8.87 (s, 1H), 8.47 (s, 1H), 7.39-7.27 (m, 4H), 7.26-7.21 (m, 4H), 7.16-7.08 (m, 3H), 5.10-4.99 (m, 1H), 4.35-4.27 (m, 1H), 4.12 (s, 2H), 3.82-3.75 (m, 1H), 2.72-2.63 (m, 5H), 2.59-2.51 (m, 5H), 2.29-2.18 (m, 1H), 2.09-2.04 (m, 1H), 1.68-1.47 (m, 3H). Chiral-SFC [Column Name: CHIRALPACK IF-3 50*3.0 mm,3.0 μm Co Solvent: B: MeOH (0.2% MSA), Start Conc. Of Pump B: 30.0%; Oven Temperature: 35° C.; Total Flow: 2.00 mL/min]: T_{R}=1.75 min (3 min run), single peak.

### Synthesis of (15,2R,3R,5R)-3-(((3-(Phenethylamino)propyl)amino)methyl)-5-(5-(1-(phenylsulfonyl)-1H-pyrazol-3-yl)-7H-pyrrolo[2,3-d]pyrimidin-7-yl)cyclopentane-1,2-diol (614)

### tert-Butyl-(3-((tert-butoxycarbonyl)(phenethyl)amino)propyl)(((3aR,4R,6R,6aS)-2,2-dimethyl-6-(5-(1-(phenylsulfonyl)-1H-pyrazol-3-yl)-7H-pyrrolo[2,3-d]pyrimidin-7-yl)tetrahydro-4H-cyclopenta[d][1,3]dioxol-4-yl)methyl)carbamate (779r)

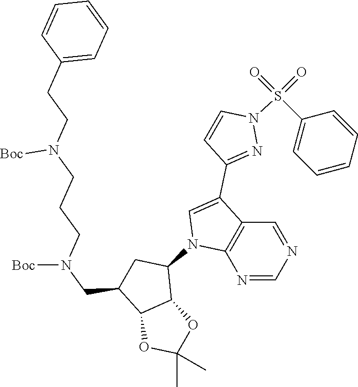 [SMILES: CC(C)(C)OC(=O)N(CCCN(C[C@H]1C[C@@H](n2cc(-c3ccn(S(=O)(=O)c4ccccc4)n3)c3cncnc32)[C@@H]2OC(C)(C)O[C@H]12)C(=O)OC(C)(C)C)CCc1ccccc1]

To a solution of tert-butyl-N-[3-({[(3aR,4R,6R,6aS)-6-{5-bromopyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 220c (60 mg, 0.08 mmol, 1.0 eq) and 1-(benzenesulfonyl)pyrazol-3-ylboronic acid 204 (31 mg, 0.12 mmol, 1.5 eq) in dioxane (2.0 mL) and H₂O (0.2 mL) were added K₃PO₄ (52 mg, 0.25 mmol, 3.0 eq) and Pd (DtBPF) Cl₂ (10 mg, 0.02 mmol, 0.2 eq) and the reaction mixture was stirred at 100° C. for 1 h, under nitrogen. The reaction mixture was concentrated under reduced pressure and the crude residue was purified by silica gel column chromatography (1:1 Petroleum ether: AcOEt) to afford tert-butyl-N-[3-({[(3aR,4R,6R,6aS)-6-{5-[1-(benzenesulfonyl)pyrazol-3-yl]pyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 229c (50 mg, 71%) as a yellow solid. LCMS [column: L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min, 1.20 mL/min, ES, m/z]: T_{R}=2.61 min; [M+H]⁺: 856.

### (1S,2R,3R,5R)-3-(((3-(Phenethylamino)propyl)amino)methyl)-5-(5-(1-(phenylsulfonyl)-1H-pyrazol-3-yl)-7H-pyrrolo[2,3-d]pyrimidin-7-yl)cyclopentane-1,2-diol (614)

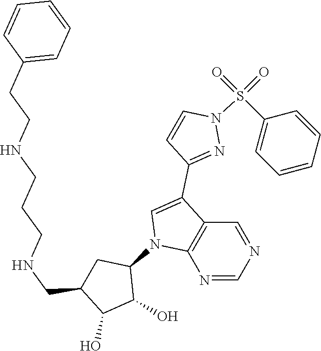 [SMILES: O=S(=O)(c1ccccc1)n1ccc(-c2cn([C@@H]3C[C@H](CNCCCNCCc4ccccc4)[C@@H](O)[C@H]3O)c3ncncc23)n1]

To tert-butyl-N-[3-({[(3aR,4R,6R,6aS)-6-{5-[1-(benzenesulfonyl)pyrazol-3-yl]pyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 229c (55 mg, 0.06 mmol, 1.0 eq) was added TFA (2.0 mL) at room temperature and the reaction mixture was stirred at 50° C. for 1 h. The resulting mixture was concentrated under reduced pressure and was adjusted to pH 8 with NH₃: H₂O. The crude residue was purified by Prep-HPLC [Column, XBridge Shield RP18 OBD Column, 19*150 mm, 5 μm; mobile phase, Water (10 mmol/L NH₄HCO₃+0.1% NH₃·H₂O) and ACN (20% ACN up to 68% in 6.5 min); Detector, UV 254 nm]: to afford (1R,2S,3R,5R)-3-{5-[1-(benzenesulfonyl)pyrazol-3-yl]pyrrolo[2,3-d]pyrimidin-7-yl}-5-[{{3-[(2-phenylethyl)amino]propyl}amino)methyl]cyclopentane-1,2-diol 614 (13.6 mg, 34%) as an off-white solid. LCMS [column: ZORBAX SB-Aq, 50*4.6 mm, 1.8 μm. mobile phase A: Water/0.02% TFA, mobile phase B: Acetonitrile, 5%-95% B-3 min, 1.50 mL/min, ES, m/z]: T_{R}=1.27 min; [M+H]⁺: 616. 1H NMR (400 MHZ, DMSO-d₆+D₂O) δ 9.31 (s, 1H), 8.85 (s, 1H), 8.52 (d, J=2.9 Hz, 1H), 8.40 (s, 1H), 8.10-7.98 (m, 2H), 7.80-7.72 (m, 1H), 7.71-7.62 (m, 2H), 7.27-7.04 (m, 6H), 5.10-4.95 (m, 1H), 4.29-4.18 (m, 1H), 3.82-3.74 (m, 1H), 2.69-2.63 (m, 10H), 2.28-2.15 (m, 1H), 2.15-1.94 (m, 1H), 1.59-1.50 (m, 3H). Chiral-HPLC [Column Name: YMC Cellulose-SB, 100*4.6 mm, 3 μm Co Solvent: B: ACN, Start Conc. of Pump B: 10.0%; Oven Temperature: 30° C.; Total Flow: 1.00 mL/min]: T_{R}=3.20 min (8 min run), single peak.

### Synthesis of (1R,2S,3R,5R)-3-(5-(4-methoxythiazol-2-yl)-7H-pyrrolo[2,3-d]pyrimidin-7-yl)-5-(((3-(phenethylamino)propyl)amino)methyl)cyclopentane-1,2-diol (615)

### tert-Butyl-(3-((tert-butoxycarbonyl)(((3aR,4R,6R,6aS)-6-(5-(4-methoxythiazol-2-yl)-7H-pyrrolo[2,3-d]pyrimidin-7-yl)-2,2-dimethyltetrahydro-4H-cyclopenta[d][1,3]dioxol-4-yl)methyl)amino)propyl)(phenethyl)carbamate (230c)

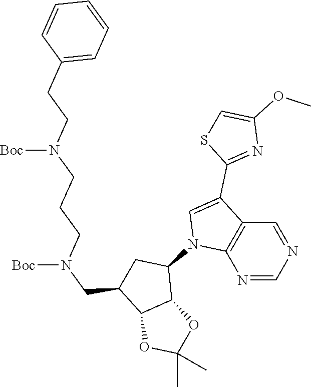 [SMILES: COc1csc(-c2cn([C@@H]3C[C@H](CN(CCCN(CCc4ccccc4)C(=O)OC(C)(C)C)C(=O)OC(C)(C)C)[C@H]4OC(C)(C)O[C@H]43)c3ncncc23)n1]

To tert-butyl-N-[3-({[(3aR,4R,6R,6aS)-6-{5-iodopyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 226c (80 mg, 0.10 mmol, 1.0 eq), Pd₂ (dba)₃ (19 mg, 0.02 mmol, 0.2 eq), tris(furan-2-yl)phosphane (9.6 mg, 0.04 mmol, 0.4 eq), K₃PO₄ (65 mg, 0.31 mmol, 3.0 eq) in THF (2.0 mL) was added (4-methoxythiazol-2-yl) zinc (II) chloride (5.0 mL in THF, crude) at room temperature and the reaction mixture was stirred at 60° C., for 12 h, under nitrogen. The resulting mixture was partitioned between EtOAc (20.0 mL) and H₂O (6.0 mL) and the organic layer was further washed with H₂O (3×10 mL), dried with anhydrous Na₂SO₄ and then concentrated under reduced pressure. The crude residue was purified by silica gel column chromatography (1:1 Petroleum ether: AcOEt) to afford tert-butyl-N-[3-({[(3aR,4R,6R,6aS)-6-[5-(4-methoxy-1,3-thiazol-2-yl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate 230c (51 mg, 62%) as a yellow solid. LCMS [column: Xbridge Shield C18, 50*3.0 mm, 3.5 μm, Mobile Phase A: Water/0.05% Ammonia, Mobile Phase B: Acetonitrile, 5%-100% B-3 min, 1.20 mL/min, ES, m/z]: T_{R}=2.24 min; [M+H]⁺: 763.

### (4-Methoxythiazol-2-yl) zinc (II) chloride

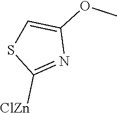

To a solution of 4-methoxythiazole (0.30 g, 1.57 mmol, 1.0 eq) [J. Am. Chem. Soc., 2013, 135 (5), 1986-1996]in THF (3.0 mL) was added n-BuLi (2.5 M in hexanes, 0.70 mL, 1.1 eq), at −78° C., under nitrogen and the resulting mixture was stirred at −78° C. for 30 min. Then zinc chloride (0.7 M solution in THF, 3.4 mL, 2.36 mmol, 1.5 eq) was added at −78° C. and the reaction mixture was stirred for 30 min at −40° C. The resulting mixture was used in the next step directly without purification.

### (1R,2S,3R,5R)-3-(5-(4-Methoxythiazol-2-yl)-7H-pyrrolo[2,3-d]pyrimidin-7-yl)-5-(((3-(phenethylamino)propyl)amino)methyl)cyclopentane-1,2-diol (615)

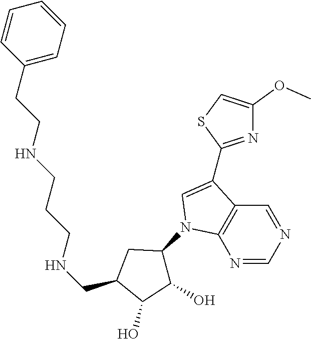 [SMILES: COc1csc(-c2cn([C@@H]3C[C@H](CNCCCNCCc4ccccc4)[C@@H](O)[C@H]3O)c3ncncc23)n1]

To tert-butyl-N-[3-({[(3aR,4R,6R,6aS)-6-[5-(4-methoxy-1,3-thiazol-2-yl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate 230c (51 mg, 0.07 mmol, 1.0 eq) was added TFA (2.0 mL) at room temperature and the reaction mixture was stirred at 40° C. for 1 h. The resulting mixture was concentrated under reduced pressure and adjusted to pH 8 with NH₃: H₂O. The crude residue was purified by Prep-HPLC [Column, XBridge Shield RP18 OBD Column, 19*150 mm, 5 μm; mobile phase, Water (10 mmol/L NH₄HCO₃+0.1% NH₃·H₂O) and ACN (20% ACN up to 72% in 8 min); Detector, UV 254 nm]: to afford (1R,2S,3R,5R)-3-[5-(4-methoxy-1,3-thiazol-2-yl)pyrrolo[2,3-d]pyrimidin-7-yl]-5-[{{3-[(2-phenylethyl)amino]propyl}amino)methyl]cyclopentane-1,2-diol 615 (14.9 mg, 42%) as an off-white solid. LCMS [column: ZORBAX SB-Aq, 50*4.6 mm, 1.8 μm. mobile phase A: Water/0.02% TFA, mobile phase B: Acetonitrile, 5%-95% B-3 min, 1.50 mL/min, ES, m/z]: T_{R}=1.15 min; [M+H]⁺: 523. 1H NMR (400 MHZ, DMSO-d₆+D₂O) δ 9.42 (s, 1H), 8.89 (s, 1H), 8.54 (s, 1H), 7.28-7.20 (m, 2H), 7.19-7.11 (m, 3H), 6.49 (s, 1H), 5.13-5.02 (m, 1H), 4.92-4.88 (m, 1H), 4.39-4.31 (m, 1H), 3.90 (s, 3H), 3.84-3.78 (m, 1H), 2.85-2.53 (m, 10H), 2.31-2.19 (m, 1H), 2.10-2.06 (m, 1H), 1.73-1.47 (m, 3H). Chiral-HPLC [Column Name: CHIRALPAK IA-3, 50*4.6 mm, 3 μm IA30CC-WE011 Co Solvent: B: Ethanol (0.2% MSA), Start Conc. of Pump B: 50.0%; Oven Temperature: 25° C.; Total Flow: 1.00 mL/min]: T_{R}=1.79 min (6 min run), single peak.

### Synthesis of ((1S,2R,3R,5R)-3-[{{3-[(2-phenylethyl)amino]propyl}(1H-pyrazol-4-yl)amino)methyl]-5-[5-(thiophen-2-yl)pyrrolo[2,3-d]pyrimidin-7-yl]cyclopentane-1,2-diol (615)

### [(3aR,4R,6R,6aS)-2,2-Dimethyl-6-{pyrrolo[2,3-d]pyrimidin-7-yl}-tetrahydro-3aH-cyclopenta[d][1,3]dioxol-4-yl]methanol (231c)

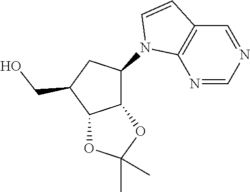 [SMILES: CC1(C)O[C@@H]2[C@@H](CO)C[C@@H](n3ccc4cncnc43)[C@@H]2O1]

To a solution of [(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]methanol (5.00 g, 15.4 mmol, 1.0 eq) in MeOH (50.0 mL) was added Pd/C (1.64 g, 15.4 mmol, 0.1 eq, 10%) and the reaction mixture was stirred for 1 h at room temperature under a H₂ atmosphere (40 psi). The resulting mixture was filtered, the filter cake was washed with MeOH (3×20 mL) and the filtrate was concentrated under reduced pressure. The crude residue was dissolved in DCM (5.0 mL) and was purified by silica gel column chromatography (2:3 Petroleum ether: THF) to afford [(3aR,4R,6R,6aS)-2,2-dimethyl-6-{pyrrolo[2,3-d]pyrimidin-7-yl}-tetrahydro-3aH-cyclopenta[d][1,3]dioxol-4-yl]methanol 231c (1.50 g, 33%) as a pale yellow oil. LCMS [conditions Proshell HPH-C18, 50*3.0 mm, 2.7 μm, Mobile Phase A: Water (0.05% ammonia water), Mobile Phase B: Acetonitrile, 2%-100% B-2 min (+), 1.50 L/min, ES, m/z]: T_{R}=1.01 min; [M+H]⁺: 290.1H NMR (300 MHZ, DMSO-d₆) δ9.43 (s, 1H), 9.20 (s, 1H), 8.27 (d, J=3.4 Hz, 1H), 7.06 (d, J=3.2 Hz, 1H), 5.31-5.10 (m, 1H), 4.99-4.84 (m, 1H), 4.66-4.53 (m, 1H), 3.61-3.51 (m, 2H), 2.36-2.21 (m, 2H), 2.20-1.95 (m, 1H), 1.49 (s, 3H), 1.23 (s, 3H).

### [(3aR,4R,6R,6aS)-6-{5-Bromopyrrolo[2,3-d]pyrimidin-7-yl}-2,2-dimethyl-tetrahydro-3aH-cyclopenta[d][1,3]dioxol-4-yl]methanol (232c)

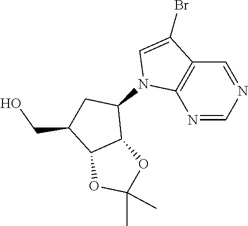 [SMILES: CC1(C)O[C@@H]2[C@@H](CO)C[C@@H](n3cc(Br)c4cncnc43)[C@@H]2O1]

To a solution of [(3aR,4R,6R,6aS)-2,2-dimethyl-6-{pyrrolo[2,3-d]pyrimidin-7-yl}-tetrahydro-3aH-cyclopenta[d][1,3]dioxol-4-yl]methanol 231c (0.70 g, 2.40 mmol, 1.0 eq) in DMF (7.0 mL) was added a solution of NBS (0.51 g, 2.90 mmol, 1.2 eq) at 0° C. under N₂ and the reaction mixture was stirred at 0° C. for 30 min. The reaction was quenched by the addition of Na₂S203 aq. at 0° C. and the resulting mixture was extracted with AcOEt (3×5 mL). The combined organic extracts were washed with brine, dried over anhydrous Na₂SO₄ and filtered. The filtrate was concentrated under reduced pressure and the crude residue was purified by Prep-TLC (2:3 Petroleum ether: AcOEt) to afford [(3aR,4R,6R,6aS)-6-{5-bromopyrrolo[2,3-d]pyrimidin-7-yl}-2,2-dimethyl-tetrahydro-3aH-cyclopenta[d][1,3]dioxol-4-yl]methanol 232c (300 mg, 33%) as a pale yellow oil. LCMS [conditions Proshell HPH-C18, 50*3.0 mm, 2.7 μm, Mobile Phase A: Water (0.05% ammonia water), Mobile Phase B: Acetonitrile, 2%-100% B-2 min (+), 1.50 L/min, ES, m/z]: T_{R}=1.11 min; [M+H]⁺: 368, 370. ¹H NMR (300 MHZ, DMSO-d₆) δ8.81 (s, 1H), 8.73 (s, 1H), 6.01 (s, 1H), 5.22-5.13 (m, 1H), 4.93-4.90 (m, 1H), 4.66-4.54 (m, 2H), 3.98-3.89 (m, 1H), 2.69-2.63 (m, 1H), 2.48-2.40 (m, 1H), 1.70-1.59 (m, 1H), 1.45 (s, 3H), 1.28 (s, 3H).

### [(3aR,4R,6R,6aS)-2,2-Dimethyl-6-[5-(thiophen-2-yl)pyrrolo[2,3-d]pyrimidin-7-yl]-tetrahydro-3aH-cyclopenta[d][1,3]dioxol-4-yl]methanol (??? r)

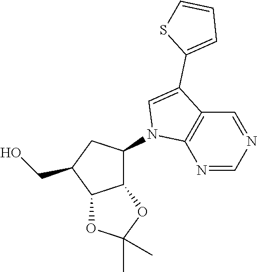 [SMILES: CC1(C)O[C@@H]2[C@@H](CO)C[C@@H](n3cc(-c4cccs4)c4cncnc43)[C@@H]2O1]

To a solution of [(3aR,4R,6R,6aS)-6-{5-bromopyrrolo[2,3-d]pyrimidin-7-yl}-2,2-dimethyl-tetrahydro-3aH-cyclopenta[d][1,3]dioxol-4-yl]methanol 232c (0.50 g, 1.30 mmol, 1.0 eq) in dioxane (2.0 mL) were added thiophen-2-ylboronic acid (0.26 g, 2.00 mmol, 1.5 eq), Pd (dtbpf) Cl₂ (88.5 mg, 0.13 mmol, 0.1 eq), K₃PO₄ (0.58 g, 2.70 mmol, 2.0 eq) and H₂O (0.2 mL) and the reaction mixture was stirred at 80° C. for 1 h, under N₂. The resulting mixture was concentrated under reduced pressure and the crude residue was purified by silica gel column chromatography (3:5 Petroleum ether: AcOEt) to afford [(3aR,4R,6R,6aS)-2,2-dimethyl-6-[5-(thiophen-2-yl)pyrrolo[2,3-d]pyrimidin-7-yl]-tetrahydro-3aH-cyclopenta[d][1,3]dioxol-4-yl]methanol 233c (350 mg, 69%) as an off-white solid. LCMS [conditions Proshell HPH-C18, 50*3.0 mm, 2.7 μm, Mobile Phase A: Water (0.05% ammonia water), Mobile Phase B: Acetonitrile, 2%-100% B-2 min (+), 1.50 L/min, ES, m/z]: T_{R}=1.53 min; [M+H]⁺: 372.

### (3aR,45,6R,6aS)-2,2-Dimethyl-6-[5-(thiophen-2-yl)pyrrolo[2,3-d]pyrimidin-7-yl]-tetrahydro-3aH-cyclopenta[d][1,3]dioxole-4-carbaldehyde (234c)

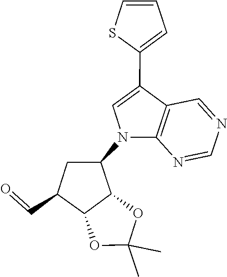 [SMILES: CC1(C)O[C@@H]2[C@H](O1)[C@@H](C=O)C[C@H]2n1cc(-c2cccs2)c2cncnc21]

To a solution of [(3aR,4R,6R,6aS)-2,2-dimethyl-6-[5-(thiophen-2-yl)pyrrolo[2,3-d]pyrimidin-7-yl]-tetrahydro-3aH-cyclopenta[d][1,3]dioxol-4-yl]methanol 233c (0.12 g, 0.30 mmol, 1.0 eq) in DCM (2.0 mL) was added DMP (0.16 g, 0.40 mmol, 1.2 eq) and the reaction mixture was stirred for 1 h at room temperature. The resulting mixture was filtered and the filtrate was concentrated under reduced pressure to afford (3aR,4S,6R,6aS)-2,2-dimethyl-6-[5-(thiophen-2-yl)pyrrolo[2,3-d]pyrimidin-7-yl]-tetrahydro-3aH-cyclopenta[d][1,3]dioxole-4-carbaldehyde 234c (0.11 g, crude) which was used in the next step without purification. LCMS [conditions L-column3 ODS, 50*3.0 mm, 3.0 μm, Mobile Phase Water/5 mM NH₄HCO₃+0.05% Ammonia, Mobile Phase B: Acetonitrile/5% water, 2%-100% B-3 min (+), 1.50 L/min, ES, m/z]: T_{R}=1.76 min; [M+H]⁺: 370.

### tert-Butyl-N-(2-phenylethyl)-NV-[3-(1H-pyrazol-4-ylamino)propyl]carbamate (235r)

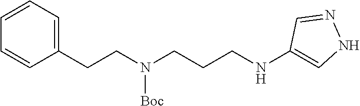 [SMILES: CC(C)(C)OC(=O)N(CCCNc1cn[nH]c1)CCc1ccccc1]

To a solution of 4-aminopyrazole (71.9 mg, 0.80 mmol, 1.2 eq) in DCM (1.5 mL) were added AcOH (43.3 mg, 0.70 mmol, 1.0 eq) and NaBH (AcO) 3 (0.30 g, 1.40 mmol, 2.0 eq) and the resulting mixture was stirred for 30 min at 0° C. To the above mixture was added tert-butyl-N-(3-oxopropyl)-N-(2-phenylethyl)carbamate 71c (0.20 g, 0.70 mmol, 1.0 eq) in DCM (0.5 mL), dropwise over 3 min, at 0° C. and the reaction mixture was stirred for additional 30 min at 0° C. The resulting mixture was concentrated under reduced pressure and was purified by silica gel column chromatography (96:4 DCM: MeOH) to afford tert-butyl-N-(2-phenylethyl)-N-[3-(1H-pyrazol-4-ylamino)propyl]carbamate 235c (80 mg, 32%) as a colorless oil. LCMS [conditions L-column3 ODS, 50*3.0 mm, 3.0 μm, Mobile PhaseA: Water/5 mM NH₄HCO₃+0.05% Ammonia, Mobile Phase B: Acetonitrile/5% water, 2%-100% B-2 min (+), 1.50 L/min, ES, m/z]: T_{R}=1.33 min; [M+H]⁺: 345.

### tert-Butyl-N-[3-({[(3aR,4R,6R,6aS)-2,2-dimethyl-6-[5-(thiophen-2-yl)pyrrolo[2,3-d]pyrimidin-7-yl]-tetrahydro-3aH-cyclopenta[d][1,3]dioxol-4-yl]methyl}(1H-pyrazol-4-yl)amino)propyl]-N-(2-phenylethyl)carbamate (236c)

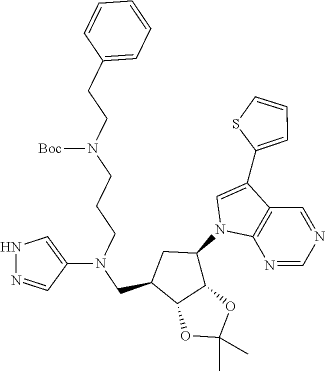 [SMILES: CC(C)(C)OC(=O)N(CCCN(C[C@H]1C[C@@H](n2cc(-c3cccs3)c3cncnc32)[C@@H]2OC(C)(C)O[C@H]12)c1cn[nH]c1)CCc1ccccc1]

To a solution of tert-butyl-N-(2-phenylethyl)-N-[3-(1H-pyrazol-4-ylamino)propyl]carbamate 235c (0.15 g, 0.40 mmol, 1.5 eq) in DCM (2.0 mL) were added AcOH (0.18 g, 3.00 mmol, 10 eq) and NaBH (OAc) 3 (0.13 g, 0.60 mmol, 2.0 eq) and the resulting mixture was stirred for 30 min at 0° C. To the above mixture was added (3aR,4S,6R,6aS)-2,2-dimethyl-6-[5-(thiophen-2-yl)pyrrolo[2,3-d]pyrimidin-7-yl]-tetrahydro-3aH-ccyclopenta[d][1,3]dioxole-4-carbaldehyde 234c (0.11 g, 0.30 mmol, 1.0 eq) in DCM (2.0 mL), dropwise over 3 min, at 0° C. and the reaction mixture was stirred for an additional 30 min at 0° C. The resulting mixture was concentrated under reduced pressure and the crude residue was purified by Prep-HPLC [Column: Welch Xtimate C18 ExRS, 250 mm, 10 μm; Mobile Phase A: Water (0.05% NH₃·H₂O), Mobile Phase B: ACN; Flow rate: 90 mL/min; Gradient: 90% B to 90% B in 10 min, 90% B; Wave Length: 254 nm]: to afford tert-butyl-N-[3-({[(3aR,4R,6R,6aS)-2,2-dimethyl-6-[5-(thiophen-2-yl)pyrrolo[2,3-d]pyrimidin-7-yl]-tetrahydro-3aH-cyclopenta[d][1,3]dioxol-4-yl]methyl}(1H-pyrazol-4-yl)amino)propyl]-N-(2-phenylethyl)carbamate 236 (80 mg, 38%) as an off-white solid. LCMS [conditions L-column3 ODS, 50*3.0 mm, 3.0 μm, Mobile Phase Water/5 mM NH₄HCO₃+0.05% Ammonia, Mobile Phase B: Acetonitrile/5% water, 2%-100% B-2 min (+), 1.50 L/min, ES, m/z]: T_{R}=2.22 min; [M+H]⁺: 698.

### ((1S,2R,3R,5R)-3-[{{3-[(2-Phenylethyl)amino]propyl}(1H-pyrazol-4-yl)amino)methyl]-5-[5-(thiophen-2-yl)pyrrolo[2,3-d]pyrimidin-7-yl]cyclopentane-1,2-diol (616)

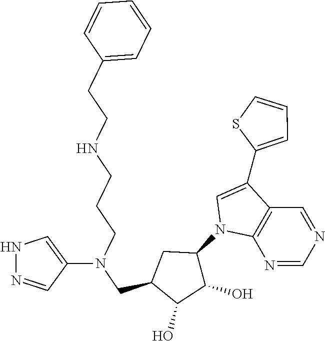 [SMILES: O[C@@H]1[C@@H](CN(CCCNCCc2ccccc2)c2cn[nH]c2)C[C@@H](n2cc(-c3cccs3)c3cncnc32)[C@@H]1O]

To tert-butyl-N-[3-({[(3aR,4R,6R,6aS)-2,2-dimethyl-6-[5-(thiophen-2-yl)pyrrolo[2,3-d]pyrimidin-7-yl]-tetrahydro-3aH-cyclopenta[d][1,3]dioxol-4-yl]methyl}(1H-pyrazol-4-yl)amino)propyl]-N-(2-phenylethyl)carbamate 236c (30 mg, 0.043 mmol, 1.0 eq) was added 4M HCl in MeOH (2.0 mL) and the reaction mixture was stirred at 50° C. for 10 min. The resulting mixture was concentrated under reduced pressure and the crude residue was purified by Prep-HPLC [Column: YMC-Actus Triart C18, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 20% B to 60% B in 10 min, 55% B; Wave Length: 254 nm]: to afford ((1S,2R,3R,5R)-3-[{{3-[(2-phenylethyl)amino]propyl}(1H-pyrazol-4-yl)amino)methyl]-5-[5-(thiophen-2-yl)pyrrolo[2,3-d]pyrimidin-7-yl]cyclopentane-1,2-diol 616 (12.3 mg, 51%) as a pale yellow solid. LCMS [ZORBAX SB-Aq, 50*4.6 mm, 1.8 μm, Mobile Phase A:

Water/0.02% TFA, Mobile Phase B: Acetonitrile, 5%-95% B-3 min, 1.50 L/min, ES, m/z]: T_{R}=1.33 min; [M+H]⁺: 558. ¹H NMR (400 MHZ, DMSO-d₆+D₂O) δ9.56 (s, 1H), 9.16 (s, 1H), 8.54 (s, 1H), 7.81 (s, 2H), 7.65-7.56 (m, 2H), 7.37-7.29 (m, 2H), 7.28-7.16 (m, 4H), 5.15-5.03 (m, 1H), 4.41-4.27 (m, 1H), 3.93-3.85 (m, 1H), 3.56-3.49 (m, 1H), 3.48-3.32 (m, 3H), 3.17-3.04 (m, 2H), 3.02-2.94 (m, 2H), 2.94-2.84 (m, 2H), 2.30-2.12 (m, 2H), 1.97-1.80 (m, 2H), 1.79-1.64 (m, 1H).

### Synthesis of (1R,2S,3R,5R)-3-[5-(3-benzyl-1,2-thiazol-5-yl)pyrrolo[2,3-d]pyrimidin-7-yl]-5-[({3-[(2-phenylethyl)amino]propyl}amino)methyl]cyclopentane-1,2-diol (617)

### N-[(12)-(5-Bromo-1,2-thiazol-3-yl)methylidene]-4-methylbenzenesulfonohydrazide (237c)

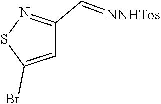

To a solution of 5-bromo-1,2-thiazole-3-carbaldehyde (2.20 g, 11.4 mmol, 1.0 eq) in DME (20 mL) was added 4-toluenesulfonyhydrazide (3.20 g, 17.1 mmol, 1.5 eq) and the reaction mixture was stirred for 30 min at room temperature. The resulting mixture was concentrated under reduced pressure and the crude residue was purified by silica gel column chromatography (3:2 Petroleum ether: AcOEt) to afford N-[(12)-(5-bromo-1,2-thiazol-3-yl)methylidene]-4-methylbenzenesulfonohydrazide 237c (1.00 g, 24%) as a pale yellow solid. LCMS [conditions L-column3 ODS, 50*3.0 mm, 3.0 μm, Mobile Phase Water/5 mM NH₄HCO₃+0.05% Ammonia, Mobile Phase B: Acetonitrile/5% water, 2%-100% B-5 min (+), 1.50 L/min, ES, m/z]: T_{R}=1.94 min; [M+H]⁺: 360, 362.

### 3-Benzyl-5-bromo-1,2-thiazole (238) [2090331-44-3/Enamine]

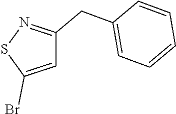 [SMILES: Brc1cc(Cc2ccccc2)ns1]

To a solution of N-[(1Z)-(5-bromo-1,2-thiazol-3-yl)methylidene]-4-methylbenzenesulfonohydrazide 237c (0.20 g, 0.10 mmol, 1.0 eq) in dioxane (3.0 mL) were added phenyl boronic acid (0.10 g, 0.80 mmol, 1.5 eq) and Cs₂CO₃ (362 mg, 1.10 mmol, 2.0 eq) and the reaction mixture was stirred at 70° C. for 20 min. The resultant mixture was allowed to cool down to room temperature, filtered and the filtrate was concentrated under reduced pressure. The crude residue was purified by Prep-TLC (12:1 Petroleum ether: AcOEt) to afford 3-benzyl-5-bromo-1,2-thiazole 238c (0.14 g, 99%) as a colorless oil. LCMS [conditions Halo C18, 50*3.0 mm, 2.7 μm, Mobile Phase A: Water/0.05% TFA, Mobile Phase B: Water/0.05% TFA, 2%-100% B-3 min (+), 1.50 L/min, ES, m/z]: T_{R}=1.85 min; [M+H]⁺: 254, 256

### tert-Butyl-N-[3-({[(3aR,4R,6R,6aS)-2,2-dimethyl-6-[5-(4,4,5,5-tetramethyl-1,3,2-dioxaborolan-2-yl)pyrrolo[2,3-d]pyrimidin-7-yl]-tetrahydro-3aH-cyclopenta[d][1,3]dioxol-4-yl]methyl}(tert-butoxycarbonyl)amino)propyl]-N-(2-phenylethyl)carbamate (239c)

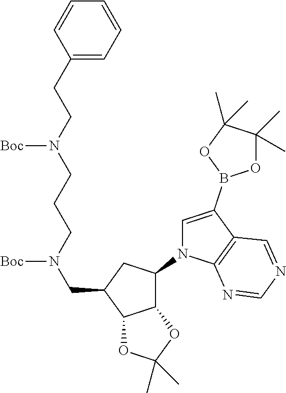 [SMILES: CC(C)(C)OC(=O)N(CCCN(C[C@H]1C[C@@H](n2cc(B3OC(C)(C)C(C)(C)O3)c3cncnc32)[C@@H]2OC(C)(C)O[C@H]12)C(=O)OC(C)(C)C)CCc1ccccc1]

To a solution of tert-butyl-N-[3-({[(3aR,4R,6R,6aS)-6-{5-bromopyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 220c (150 mg, 0.20 mmol, 1.0 eq) in dioxane (1.5 mL) was added bis(pinacolato)diboron (62.7 mg, 0.20 mmol, 1.2 eq), Pd (dppf) Cl₂ (15.1 mg, 0.021 mmol, 0.1 eq), and AcOK (40.40 mg, 0.4 mmol, 2.0 eq) and the reaction mixture was stirred at 100° C. for 1 h, under N₂. The resulting mixture was concentrated under vacuum to afford tert-butyl-N-[3-({[(3aR,4R,6R,6aS)-2,2-dimethyl-6-[5-(4,4,5,5-tetramethyl-1,3,2-dioxaborolan-2-yl)pyrrolo[2,3-d]pyrimidin-7-yl]-tetrahydro-3aH-cyclopenta[d][1,3]dioxol-4-yl]methyl}(tert-butoxycarbonyl)amino)propyl]-N-(2-phenylethyl)carbamate 239c (150 mg, crude) which was used in the next step without purification. LCMS [Conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-95% B-3 min, 1.50 L/min, ES, m/z]: T_{R}=2.64 min; [M+H]⁺: 776

### tert-Butyl-N-[3-({[(3aR,4R,6R,6aS)-6-[5-(3-benzyl-1,2-thiazol-5-yl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate (240c)

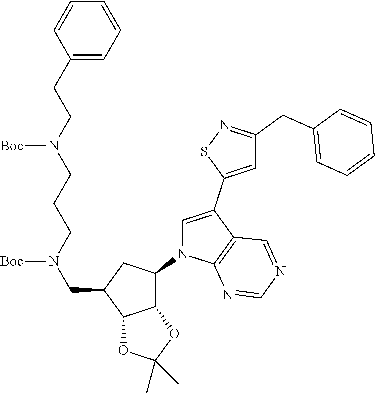 [SMILES: CC(C)(C)OC(=O)N(CCCN(C[C@H]1C[C@@H](n2cc(-c3cc(Cc4ccccc4)ns3)c3cncnc32)[C@@H]2OC(C)(C)O[C@H]12)C(=O)OC(C)(C)C)CCc1ccccc1]

To a solution of tert-butyl-N-[3-({[(3aR,4R,6R,6aS)-2,2-dimethyl-6-[5-(4,4,5,5-tetramethyl-1,3,2-dioxaborolan-2-yl)pyrrolo[2,3-d]pyrimidin-7-yl]-tetrahydro-3aH-cyclopenta[d][1,3]dioxol-4-yl]methyl}(tert-butoxycarbonyl)amino)propyl]-N-(2-phenylethyl)carbamate 239c (150 mg, 0.20 mmol, 1.0 eq) in dioxane (1.0 mL) and H₂O (0.2 mL) was added 3-benzyl-5-bromo-1,2-thiazole (73.7 mg, 0.30 mmol, 1.5 eq), Pd (dppf) Cl₂ (14.2 mg, 0.019 mmol, 0.1 eq) and AcOK (38.0 mg, 0.40 mmol, 2.0 eq), and the reaction mixture was stirred at 80° C. for 2 h. The resulting mixture was diluted with water (10 mL) and extracted with AcOEt (3×10 mL). The combined organic layers were washed with brine, dried over anhydrous Na₂SO₄ and filtered. The filtrate was concentrated under reduced pressure and was purified by Prep-TLC (7:3 Petroleum ether: THF) to afford tert-butyl-N-[3-({[(3aR,4R,6R,6aS)-6-[5-(3-benzyl-1,2-thiazol-5-yl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate 240c (30 mg, 19%) as a yellow oil. LCMS [Conditions Kinetex XB-C18, 50*3.0 mm, 2.6 μm, Mobile Phase A: Water/0.02% TFA, Mobile Phase B: Acetonitrile, 2%-95% B-5 min (+), 1.50 L/min]: T_{R}=3.59 min; [M+H]⁺: 823.

### (1R,2S,3R,5R)-3-[5-(3-Benzyl-1,2-thiazol-5-yl)pyrrolo[2,3-d]pyrimidin-7-yl]-5-[({3-[(2-phenylethyl)amino]propyl}amino)methyl]cyclopentane-1,2-diol (617)

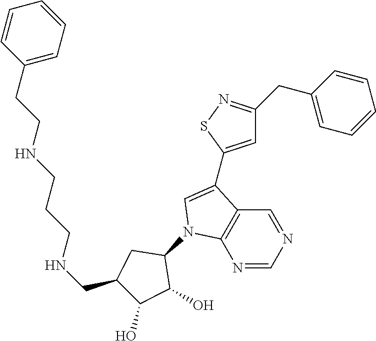 [SMILES: O[C@@H]1[C@@H](CNCCCNCCc2ccccc2)C[C@@H](n2cc(-c3cc(Cc4ccccc4)ns3)c3cncnc32)[C@@H]1O]

To tert-butyl-N-[3-({[(3aR,4R,6R,6aS)-6-[5-(3-benzyl-1,2-thiazol-5-yl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate 240c (30 mg, 0.04 mmol, 1.0 eq) was added 4M HCl in MeOH (2.0 mL) and the reaction mixture was stirred at 50° C. for 10 min. The resulting mixture was concentrated under reduced pressure and the crude residue was purified by Prep-HPLC [Column: YMC-Actus Triart C18, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 20% B to 60% B in 10 min, 55% B; Wave Length: 254 nm]: to afford (1R,2S,3R,5R)-3-[5-(3-benzyl-1,2-thiazol-5-yl)pyrrolo[2,3-d]pyrimidin-7-yl]-5-[({3-[(2-phenylethyl)amino]propyl}amino)methyl]cyclopentane-1,2-diol 617 (3.8 mg, 18%) as an off-white solid. LCMS [ZORBAX SB-Aq, 50*4.6 mm, 1.8 μm, Mobile Phase A: Water/0.02% TFA, Mobile Phase B: Acetonitrile, 5%-95% B-3 min, 1.50 L/min, ES, m/z]: T_{R}=1.34 min; [M+H]⁺: 583. ¹H NMR (400 MHZ, DMSO-d₆) δ9.29 (s, 1H), 8.91 (s, 1H), 8.47 (s, 1H), 7.67 (s, 1H), 7.39-7.29 (m, 4H), 7.29-7.18 (m, 3H), 7.20-7.11 (m, 3H), 5.13-5.02 (m, 1H), 4.34-4.26 (m, 1H), 4.17 (s, 2H), 3.85-3.78 (m, 1H), 2.86-2.54 (m, 10H), 2.31-2.20 (m, 1H), 2.11-2.06 (m, 1H), 1.66-1.53 (m, 3H).

### Synthesis of (1R,2S,3R,5R)-3-[5-(4-Benzylthiophen-2-yl)pyrrolo[2,3-d]pyrimidin-7-yl]-5-{[(3-{[2-(4-fluorophenyl)ethyl]amino}propyl)amino]methyl}cyclopentane-1,2-diol (618)

### tert-Butyl-N-{[(3aR,4R,6aR)-2,2-dimethyl-6-oxo-tetrahydrocyclopenta[d][1.3]dioxol-4-yl]methyl}carbamate (241c)

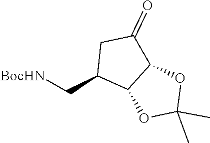 [SMILES: CC(C)(C)OC(=O)NC[C@H]1CC(=O)[C@@H]2OC(C)(C)O[C@H]12]

To a solution of (3aR,6aR)-2,2-dimethyl-3aH,6aH-cyclopenta[d][1,3]dioxol-4-one (1.50 g, 9.63 mmol, 1.0 eq) in DMF (15 mL) was added [(tert-butoxycarbonyl)amino]acetic acid (2.56 g, 14.6 mmol, 1.5 eq), IR [DF (CF₃) PPY]2 (DTBPY) PF₆ (109 mg, 0.01 mmol, 0.01 eq) and K₂HPO₄ (3.29 g, 19.9 mmol, 2.0 eq) and the reaction mixture was stirred at room temperature for 24 h, under N₂, with blue LED (60 W). The resulting mixture was diluted with water (50 mL) and the resulting mixture was extracted with AcOEt (3×30 mL). The combined organic layers were washed with brine, dried over anhydrous Na₂SO₄ and then filtered. The filtrate was concentrated under reduced pressure and the crude residue was purified by silica gel column chromatography (3:1 Petroleum ether: THF) to afford tert-butyl-N-{[(3aR,4R,6aR)-2,2-dimethyl-6-oxo-tetrahydrocyclopenta[d][1,3]dioxol-4-yl]methyl}carbamate 241c (1.50 g, 54%) as a pale yellow oil. LCMS [conditions L-column3 C18, 50*3.0 mm, 3.0 μm, Mobile Phase A: Water/5 mM NH₄HCO₃, Mobile Phase B: Methanol, 2%-100% B-3 min (+), 1.50 L/min, ES, m/z]: T_{R}=2.32 min; [M+H]⁺: 286.

### tert-Butyl-N-{[(3aR,4R,6S,6aS)-6-hydroxy-2,2-dimethyl-tetrahydro-3aH-cyclopenta[d][1,3]dioxol-4-yl]methyl}carbamate (242c)

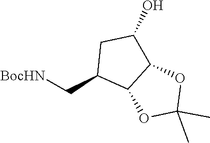 [SMILES: CC(C)(C)OC(=O)NC[C@H]1C[C@H](O)[C@@H]2OC(C)(C)O[C@H]12]

To tert-butyl-N-{[(3aR,4R,6aR)-2,2-dimethyl-6-oxo-tetrahydrocyclopenta[d][1,3]dioxol-4-yl]methyl}carbamate 241c (900 mg, 3.15 mmol, 1.0 eq) in MeOH (20 mL) was added NaBH₄ (179 mg, 4.73 mmol, 1.5 eq), in several portions, over 10 min at 0° C. and the reaction mixture was stirred for 1 h at 0° C. The resulting mixture was concentrated under reduced pressure and the crude residue was purified by silica gel column chromatography (4:1 Petroleum ether: THF) to afford tert-butyl-N-{[(3aR,4R,6S,6aS)-6-hydroxy-2,2-dimethyl-tetrahydro-3aH-cyclopenta[d][1,3]dioxol-4-yl]methyl}carbamate 242c (790 mg, 87%) as a white solid. LCMS [Cortecs C18+, 50*3.0 mm, 2.7 μm, Mobile Phase A: Water/5 mM NH₄HCO₃+0.05% Ammonia, Mobile Phase B: Acetonitrile, 5%-100% B-2 min (+−), 1.50 L/min, ES, m/z]: T_{R}=1.12 min; [M+H]⁺: 288.

### tert-Butyl-N-{[(3aR,4R,6R,6aS)-6-{5-bromopyrrolo[2,3-d]pyrimidin-7-yl}-2,2-dimethyl-tetrahydro-3aH-cyclopenta[d][1,3]dioxol-4-yl]methyl}carbamate (243c)

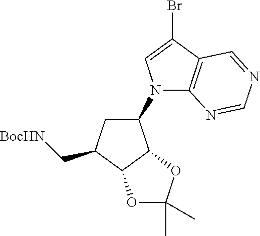 [SMILES: CC(C)(C)OC(=O)NC[C@H]1C[C@@H](n2cc(Br)c3cncnc32)[C@@H]2OC(C)(C)O[C@H]12]

To a solution of tert-butyl-N-{[(3aR,4R,6S,6aS)-6-hydroxy-2,2-dimethyl-tetrahydro-3aH-cyclopenta[d][1,3]dioxol-4-yl]methyl}carbamate 242c (400 mg, 1.39 mmol, 1.0 eq) and PPh₃ (730 mg, 2.78 mmol, 2.0 eq) in toluene (20 mL) was added 5-bromo-7H-pyrrolo[2,3-d]pyrimidine (276 mg, 1.39 mmol, 1.0 eq) and the resulting mixture was stirred for 10 min at 0° C. To the above mixture was added DBAD (641 mg, 2.78 mmol, 2.0 eq) in toluene (1.0 mL), dropwise over 5 min at 0° C., and the reaction mixture was stirred at 60° C. for 2 h. The resulting mixture was concentrated under reduced pressure and the crude residue was purified by silica gel column chromatography (5:1 Petroleum ether: THF) to afford tert-butyl-N-{[(3aR,4R,6R,6aS)-6-{5-bromopyrrolo[2,3-d]pyrimidin-7-yl}-2,2-dimethyl-tetrahydro-3aH-cyclopenta[d][1,3]dioxol-4-yl]methyl}carbamate 243c (240 mg, 37%) as a white solid. LCMS [Cortecs C18+, 50*3.0 mm, 2.7 μm, Mobile Phase A: Water/5 mM NH₄HCO₃+0.05% Ammonia, Mobile Phase B: Acetonitrile, 5%-100% B-3 min (+−), 1.50 L/min, ES, m/z]: T_{R}=1.63 min; [M+H]⁺: 467, 469.

### tert-Butyl-N-{[(3aR,4R,6R,6aS)-6-[5-(4-benzylthiophen-2-yl)pyrrolo[2,3-d]pyrimidin-7-yl]-2,2-dimethyl-tetrahydro-3aH-cyclopenta[d][1.3]dioxol-4-yl]methyl]carbamate)(244c)

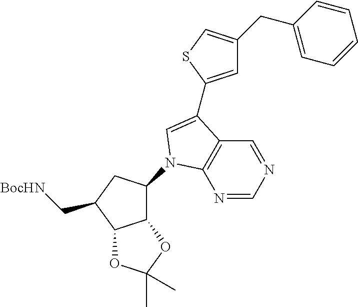 [SMILES: CC(C)(C)OC(=O)NC[C@H]1C[C@@H](n2cc(-c3cc(Cc4ccccc4)cs3)c3cncnc32)[C@@H]2OC(C)(C)O[C@H]12]

To a solution of tert-butyl-N-{[(3aR,4R,6R,6aS)-6-{5-bromopyrrolo[2,3-d]pyrimidin-7-yl}-2,2-dimethyl-tetrahydro-3aH-cyclopenta[d][1,3]dioxol-4-yl]methyl}carbamate 243c (240 mg, 0.51 mmol, 1.0 eq) and 2-(4-benzylthiophen-2-yl)-4,4,5,5-tetramethyl-1,3,2-dioxaborolane 315c (224 mg, 1.02 mmol, 2.0 eq) in dioxane (20 mL) and H₂O (2.0 mL) were added K₃PO₄ (327 mg, 1.54 mmol, 3.0 eq) and Pd (DtBPF) Cl₂ (16.7 mg, 0.03 mmol, 0.05 eq) and the reaction mixture was stirred at 90° C. for 2 h, under nitrogen. The resulting mixture was concentrated under reduced pressure and the crude residue was purified by silica gel column chromatography (3:1 Petroleum ether: THF) to afford tert-butyl-N-{[(3aR,4R,6R,6aS)-6-[5-(4-benzylthiophen-2-yl)pyrrolo[2,3-d]pyrimidin-7-yl]-2,2-dimethyl-tetrahydro-3aH-cyclopenta[d][1,3]dioxol-4-yl]methyl}carbamate) 244c (170 mg, 65%) as a colorless oil. LCMS [Cortecs C18+, 50*3.0 mm, 2.7 μm, Mobile Phase A: Water/5 mM NH₄HCO₃+0.05% Ammonia, Mobile Phase B: Acetonitrile, 5%-100% B-3 min (+−), 1.50 L/min, ES, m/z]: T_{R}=2.25 min; [M+H]⁺: 561.

### (1S,2R,3R,5R)-3-(Aminomethyl)-5-[5-(4-benzylthiophen-2-yl)pyrrolo[2,3-d]pyrimidin-7-yl]cyclopentane-1,2-diol (245c)

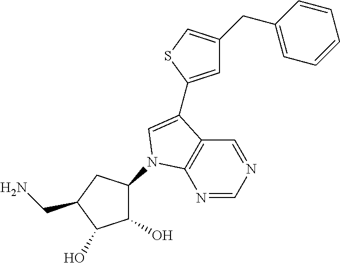 [SMILES: NC[C@H]1C[C@@H](n2cc(-c3cc(Cc4ccccc4)cs3)c3cncnc32)[C@H](O)[C@@H]1O]

To tert-butyl-N-{[(3aR,4R,6R,6aS)-6-[5-(4-benzylthiophen-2-yl)pyrrolo[2,3-d]pyrimidin-7-yl]-2,2-dimethyl-tetrahydro-3aH-cyclopenta[d][1,3]dioxol-4-yl]methyl}carbamate 244c (120 mg, 0.21 mmol, 1.0 eq) was added 4M HCl in MeOH (2.5 mL, 10 mmol) and the reaction mixture was stirred at 50° C. for 1 h. The resulting mixture was concentrated under reduced pressure to afford (1S,2R,3R,5R)-3-(aminomethyl)-5-[5-(4-benzylthiophen-2-yl)pyrrolo[2,3-d]pyrimidin-7-yl]cyclopentane-1,2-diol 245c (70 mg, crude) as a white solid. LCMS [Cortecs C18+, 50*3.0 mm, 2.7 μm, Mobile Phase A: Water/5 mM NH₄HCO₃+0.05% Ammonia, Mobile Phase B: Acetonitrile, 5%-100% B-2.5 min (+−), 1.50 L/min, ES, m/z]: T_{R}=1.31 min; [M+H]⁺: 421.

### tert-Butyl-N-[3-({[(1R,2R,3S,4R)-4-[5-(4-benzylthiophen-2-yl)pyrrolo[2,3-d]pyrimidin-7-yl]-2,3-dihydroxycyclopentyl]methyl}amino)propyl]-N-[2-(4-fluorophenyl)ethyl]carbamate (246c)

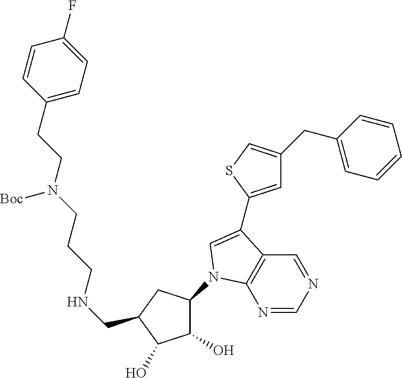 [SMILES: CC(C)(C)OC(=O)N(CCCNC[C@H]1C[C@@H](n2cc(-c3cc(Cc4ccccc4)cs3)c3cncnc32)[C@H](O)[C@@H]1O)CCc1ccc(F)cc1]

To a solution of (1S,2R,3R,5R)-3-(aminomethyl)-5-[5-(4-benzylthiophen-2-yl)pyrrolo[2,3-d]pyrimidin-7-yl]cyclopentane-1,2-diol 245c (70 mg, 0.16 mmol, 1.0 eq) and tert-butyl-N-[2-(4-fluorophenyl)ethyl]-N-(3-oxopropyl)carbamate (49.2 mg, 0.16 mmol, 1.0 eq) in DCM (5.0 mL) and MeOH (5.0 mL) was added DIEA (0.1 mL, 0.57 mmol, 3.45 eq) and the resulting mixture was stirred at 40° C. for 30 min. To this mixture was added NaBH₄ (5.7 mg, 0.24 mmol, 1.5 eq) in several portions at 0° C. and the reaction mixture was stirred at 0° C. for 0.5 h. The resulting mixture was concentrated under reduced pressure and the crude residue was purified by reverse flash chromatography [XBridge C18; mobile phase, MeCN in Ammonium bicarbonate water, 20% to 60% gradient in 10 min; detector, UV 254 nm]: to afford tert-butyl-N-[3-({[(1R,2R,3S,4R)-4-[5-(4-benzylthiophen-2-yl)pyrrolo[2,3-d]pyrimidin-7-yl]-2,3-dihydroxycyclopentyl]methyl}amino)propyl]-N-[2-(4-fluorophenyl)ethyl]carbamate 246c (56 mg, 48%) as a yellow solid. LCMS [Cortecs C18+, 50*3.0 mm, 2.7 μm, Mobile Phase A: Water/0.02% TFA, Mobile Phase B: Acetonitrile, 5%-95% B-2.5 min, 1.50 L/min, ES, m/z]: T_{R}=1.35 min; [M+H]⁺: 700.

### (1R,2S,3R,5R)-3-[5-(4-Benzylthiophen-2-yl)pyrrolo[2,3-d]pyrimidin-7-yl]-5-{[(3-{[2-(4-fluorophenyl)ethyl]amino}propyl)amino]methyl}cyclopentane-1,2-diol (618)

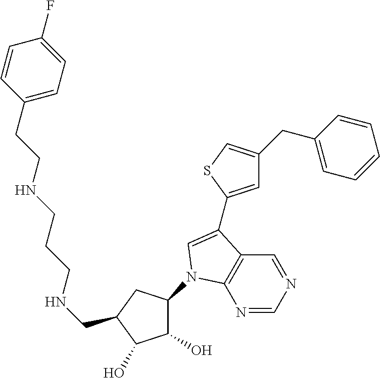 [SMILES: O[C@@H]1[C@@H](CNCCCNCCc2ccc(F)cc2)C[C@@H](n2cc(-c3cc(Cc4ccccc4)cs3)c3cncnc32)[C@@H]1O]

To tert-butyl-N-[3-({[(1R,2R,3S,4R)-4-[5-(4-benzylthiophen-2-yl)pyrrolo[2,3-d]pyrimidin-7-yl]-2,3-dihydroxycyclopentyl]methyl}amino)propyl]-N-[2-(4-fluorophenyl)ethyl]carbamate 246c (56 mg, 0.08 mmol, 1.0 eq) was added 4M HCl in MeOH (2.0 mL, 8.0 mmol) at 0° C. and the reaction mixture was then stirred at 50° C. for 10 min. The resulting mixture was concentrated under reduced pressure and the crude residue was dissolved in MeOH (2.0 mL), adjusted to PH 8 with NH₃·H₂O and the crude mixture was purified by Prep-HPLC [Column: Welch Ultimate XB-C18, 50*250 mm, 10 μm; Mobile Phase A: 0.1% HCl, Mobile Phase B: ACN; Flow rate: 90 mL/min; Gradient: 5% B-5% B-3 min, 10% B-50% B-12mim]: to afford (1R,2S,3R,5R)-3-[5-(4-benzylthiophen-2-yl)pyrrolo[2,3-d]pyrimidin-7-yl]-5-{[(3-{[2-(4-fluorophenyl)ethyl]amino}propyl)amino]methyl}cyclopentane-1,2-diol 618 (3.6 mg, 8%) as a white solid. LCMS [Cortecs C18+, 50*3.0 mm, 2.7 μm, Mobile Phase A: Water/0.02% TFA, Mobile Phase B: Acetonitrile, 5%-95% B-3 min, 1.50 L/min, ES, m/z]: T_{R}=1.38 min; [M+H]⁺: 600. ¹H NMR (300 MHZ, DMSO-d₆+D₂O) δ 9.25 (s, 1H), 8.84 (s, 1H), 8.16 (s, 1H), 7.40-7.28 (m, 5H), 7.22-7.19 (m, 3H), 7.13 (d, J=1.3 Hz, 1H), 7.11-7.00 (m, 2H), 5.13-4.98 (m, 1H), 4.87-4.84 (m, 1H), 4.32-4.27 (m, 1H), 3.97 (s, 2H), 3.81-3.79 (m, 1H), 2.84-2.54 (m, 10H), 2.28-2.15 (m, 1H), 2.09-2.07 (m, 1H), 1.68-1.51 (m, 3H). Chiral-HPLC [Column: YMC Cellulose-SB, 100*4.6 mm, 3 μm 107AB00129; Mobile Phase A: MtBE (0.2% MSA), Mobile Phase B: Ethanol (0.2% MSA); Total Flow: 1.00 mL/min; Conc. of Pump B: 60.0%; Oven Temperature: 25° C.]: T_{R}=1.90 min, single peak.

### Synthesis of (1R,2S,3R,5R)-3-(5-(4-benzylthiophen-2-yl)-7H-pyrrolo[2,3-d]pyrimidin-7-yl)-5-(((3-((4-(trifluoromethyl) phenethyl)amino)propyl)amino)methyl)cyclopentane-1,2-diol (619)

### tert-Butyl-N-[3-({[(1R,2R,3S,4R)-4-[5-(4-benzylthiophen-2-yl)pyrrolo[2,3-d]pyrimidin-7-yl]-2,3-dihydroxycyclopentyl]methyl}amino)propyl]-N-{2-[4-(trifluoromethyl)phenyl]ethyl}carbamate (247c)

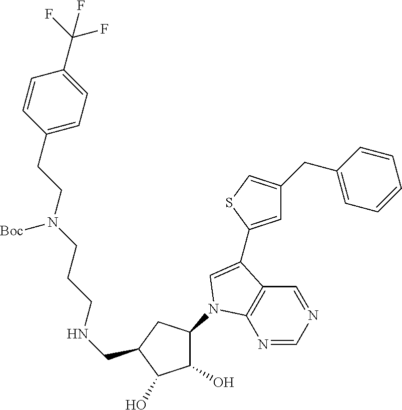 [SMILES: CC(C)(C)OC(=O)N(CCCNC[C@H]1C[C@@H](n2cc(-c3cc(Cc4ccccc4)cs3)c3cncnc32)[C@H](O)[C@@H]1O)CCc1ccc(C(F)(F)F)cc1]

To a solution of (1S,2R,3R,5R)-3-(aminomethyl)-5-[5-(4-benzylthiophen-2-yl)pyrrolo[2,3-d]pyrimidin-7-yl]cyclopentane-1,2-diol 244c (50 mg, 0.12 mmol, 1.0 eq) and tert-butyl-N-(3-oxopropyl)-N-{2-[4-(trifluoromethyl)phenyl]ethyl}carbamate 346c (41 mg, 0.12 mmol, 1.0 eq) in DCM (3.0 mL) and MeOH (3.0 mL) was added DIEA (0.1 mL, 0.57 mmol, 4.8 eq) and the resulting mixture was stirred at 40° C., for 30 min, under nitrogen. To this mixture was added NaBH₄ (9.0 mg, 0.23 mmol, 2.0 eq), in several portions at 0° C., and the reaction mixture was stirred at 0° C. for 0.5 h. The resulting mixture was concentrated under reduced pressure and the crude residue was purified by reverse flash chromatography [XBridgeC18; mobile phase, MeCN in ammonium bicarbonate water, 20% to 60% gradient in 10 min; detector, UV 254 nm]:

to afford tert-butyl-N-[3-({[(1R,2R,3S,4R)-4-[5-(4-benzylthiophen-2-yl)pyrrolo[2,3-d]pyrimidin-7-yl]-2,3-dihydroxycyclopentyl]methyl}amino)propyl]-N-{2-[4-(trifluoromethyl)phenyl]ethyl}carbamate 247c (25 mg, 28%) as a yellow solid. LCMS [Cortecs C18+, 50*3.0 mm, 2.7 μm, Mobile Phase A: Water/0.02% TFA, Mobile Phase B: Acetonitrile, 5%-95% B-2.0 min, 1.50 L/min, ES, m/z]: T_{R}=0.99 min; [M+H]⁺: 750.

### tert-butyl (3-hydroxypropyl)(4-(trifluoromethyl) phenethyl)carbamate (345c)

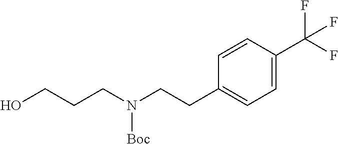 [SMILES: CC(C)(C)OC(=O)N(CCCO)CCc1ccc(C(F)(F)F)cc1]

A mixture of 1-(2-bromoethyl)-4-(trifluoromethyl)benzene (2.0 g, 7.94 mmol, 1.0 eq) and 3-aminopropan-1-ol (0.6 g, 7.94 mmol, 1.0 eq) in ethanol (20 mL) was heated at 80° C. for 6 h. The reaction mixture was then concentrated under vacuum, diluted with DCM and washed with water. The aqueous layer was extracted with DCM and the combined organic layers were washed with brine, dried over sodium sulfate, filtered, and concentrated. The residue was dissolved in DCM (20 mL). Then Et₃N (2.4 g, 23.82 mmol, 3.0 eq) and di-tert-butyl dicarbonate (2.01 g, 9.52 mmol, 1.2 eq) were added at 0° C. and the reaction mixture was stirred for 16 h at room temperature. The reaction was quenched with water (50 mL) and then extracted with DCM (3×50 mL). The organic layer was dried over anhydrous Na₂SO₄, filtered and the filtrate was concentrated under reduced pressure. The residue was purified by silica gel column chromatography (6:1 Petroleum ether: AcOEt) to afford tert-butyl (3-hydroxypropyl)(4-(trifluoromethyl) phenethyl)carbamate 345c (2.0 g, 73%) as a light yellow oil. LCMS (column: ZORBAX SB-Aq, 50*4.6 mm, 1.8 μm. mobile phase A: Water/0.02% TFA, mobile phase B: Acetonitrile, 5%-95% B-3 min, 1.50 mL/min): T_{R}=1.08 min; [M+H]⁺: 348.

### tert-butyl (3-oxopropyl)(4-(trifluoromethyl) phenethyl)carhamate (346r)

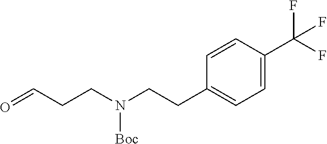 [SMILES: CC(C)(C)OC(=O)N(CCC=O)CCc1ccc(C(F)(F)F)cc1]

To tert-butyl (3-hydroxypropyl)(4-(trifluoromethyl) phenethyl)carbamate 345c (500 mg, 1.44 mmol, 1.0 eq) in DCM (5.0 mL) was added DMP (733 mg, 1.73 mmol, 1.2 eq) and the reaction mixture was stirred for 1 h at room temperature. The reaction was quenched with water (20 mL) and then extracted with DCM (3×20 mL). The organic layer was dried over anhydrous Na₂SO₄, filtered and the filtrate was concentrated under reduced pressure. The residue was purified by silica gel column chromatography (1:1 Petroleum ether: AcOEt) to afford tert-butyl (3-oxopropyl)(4-(trifluoromethyl) phenethyl)carbamate 346c (172 mg, 35%) as a colorless oil. LCMS (column: ZORBAX SB-Aq, 50*4.6 mm, 1.8 μm. mobile phase A: Water/0.02% TFA, mobile phase B: Acetonitrile, 5%-95% B-3 min, 1.50 mL/min): T_{R}=1.16 min; [M+H]⁺: 346.

### (1R,2S,3R,5R)-3-(5-(4-Benzylthiophen-2-yl)-7H-pyrrolo[2,3-d]pyrimidin-7-yl)-5-(((3-((4-(trifluoromethyl) phenethyl)amino)propyl)amino)methyl)cyclopentane-1,2-diol (619)

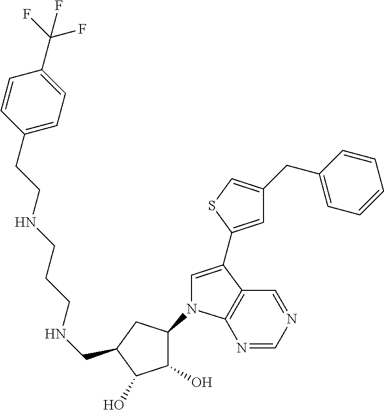 [SMILES: O[C@@H]1[C@@H](CNCCCNCCc2ccc(C(F)(F)F)cc2)C[C@@H](n2cc(-c3cc(Cc4ccccc4)cs3)c3cncnc32)[C@@H]1O]

To tert-butyl-N-[3-({[(1R,2R,3S,4R)-4-[5-(4-benzylthiophen-2-yl)pyrrolo[2,3-d]pyrimidin-7-yl]-2,3-dihydroxycyclopentyl]methyl}amino)propyl]-N-{2-[4-(trifluoromethyl)phenyl]ethyl}carbamate 247c (28 mg, 0.04 mmol, 1.0 eq) was added 4M HCl in MeOH (2.0 mL, 8.0 mmol) at 0° C. and the reaction mixture was stirred at 50° C. for 10 min. The resulting mixture was concentrated under reduced pressure, dissolved in MeOH (2.0 mL) and adjusted to pH 8 with NH₃. H₂O. The crude residue was purified by Prep-HPLC [Column: YMC-Actus Triart C18, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 20% B to 45% B in 7 min, 50% B; Wave Length: 220 nm]: to afford (1R,2S,3R,5R)-3-[5-(4-benzylthiophen-2-yl)pyrrolo[2,3-d]pyrimidin-7-yl]-5-({[3-({2-[4-(trifluoromethyl)phenyl]ethyl}amino)propyl]amino}methyl)cyclopentane-1,2-diol 619 (3.6 mg, 15%) as an off-white solid. LCMS [Cortecs C18+, 50*3.0 mm, 2.7 μm, Mobile Phase A: Water/0.02% TFA, Mobile Phase B: Acetonitrile, 5%-95% B-3 min, 1.50 L/min, ES, m/z]: T_{R}=1.43 min; [M+H]⁺: 650. ¹H NMR (400 MHZ, DMSO-d₆+D₂O) δ 9.19 (s, 1H), 8.80 (s, 1H), 8.07 (s, 1H), 7.56 (d, J=7.9 Hz, 2H), 7.37 (d, J=8.0 Hz, 2H), 7.33-7.26 (m, 5H), 7.19-7.16 (m, 1H), 7.10 (s, 1H), 5.00-5.09 (m, 1H), 4.31-4.25 (m, 1H), 3.93 (s, 2H), 3.84-3.75 (m, 1H), 2.84-2.74 (m, 5H), 2.61-2.57 (m, 5H), 2.29-2.21 (m, 1H), 2.17-2.08 (m, 1H), 1.67-1.54 (m, 3H). Chiral-HPLC [Column: CHIRALPAK IF-3, 100*4.6 mm, 3 μm IF30CS-UB006; Mobile Phase A: MtBE (0.2% MSA), Mobile Phase B: Methanol (0.2% MSA); Total Flow: 1.0000 mL/min; Conc. of Pump B: 50.0%; Oven Temperature: 25° C.]: T_{R}=1.45 min, single peak.

### Synthesis of (1R,2S,3R,5R)-3-(5-(4-benzylthiophen-2-yl)-7H-pyrrolo[2,3-d]pyrimidin-7-yl)-5-(((3-((4-(trifluoromethyl) phenethyl)amino)propyl)amino)methyl)cyclopentane-1,2-diol (620)

### tert-Butyl-N-[3-({[(1R,2R,3S,4R)-4-[5-(4-Benzylthiophen-2-yl)pyrrolo[2,3-d]pyrimidin-7-yl]-2,3-dihydroxycyclopentyl]methyl}amino)propyl]-N-(2-phenylethyl)carbamate (248c)

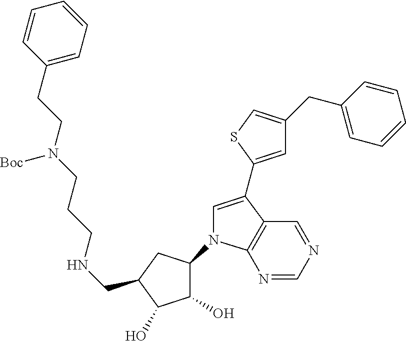 [SMILES: CC(C)(C)OC(=O)N(CCCNC[C@H]1C[C@@H](n2cc(-c3cc(Cc4ccccc4)cs3)c3cncnc32)[C@H](O)[C@@H]1O)CCc1ccccc1]

To a solution of (1S,2R,3R,5R)-3-(aminomethyl)-5-[5-(4-benzylthiophen-2-yl)pyrrolo[2,3-d]pyrimidin-7-yl]cyclopentane-1,2-diol 245c (50 mg, 0.12 mmol, 1.0 eq) and tert-butyl-N-(3-oxopropyl)-N-(2-phenylethyl)carbamate 71c (33 mg, 0.12 mmol, 1.0 eq) in DCM (5.0 mL) and MeOH (5.0 mL) was added DIEA (46 mg, 0.36 mmol, 3.0 eq) and the resulting mixture was stirred for 30 min at 40° C., under nitrogen. To this mixture was added NaBH₄ (7 mg, 0.17 mmol, 1.5 eq), in several portions, at 0° C., and the reaction mixture was stirred at 0° C. for 0.5 h. The resulting mixture was concentrated under reduced pressure and the crude residue was purified by reverse flash chromatography [XBridge C18; mobile phase, MeCN in Ammonium bicarbonate water, 20% to 60% gradient in 10 min; detector, UV 254 nm]: to afford tert-butyl-N-[3-({[(1R,2R,3S,4R)-4-[5-(4-benzylthiophen-2-yl)pyrrolo[2,3-d]pyrimidin-7-yl]-2,3-dihydroxycyclopentyl]methyl}amino)propyl]-N-(2-phenylethyl)carbamate 248c (70 mg, 86%) as a yellow solid. LCMS [Cortecs C18+, 50*3.0 mm, 2.7 μm, Mobile Phase A: Water/0.02% TFA, Mobile Phase B: Acetonitrile, 5%-95% B-2.5 min, 1.50 L/min, ES, m/z]: T_{R}=0.94 min; [M+H]⁺: 682.

### (1R,2S,3R,5R)-3-[5-(4-Benzylthiophen-2-yl)pyrrolo[2,3-d]pyrimidin-7-yl]-5-{[methyl({3-[(2-phenylethyl)amino]propyl})amino]methyl}cyclopentane-1,2-diol (620)

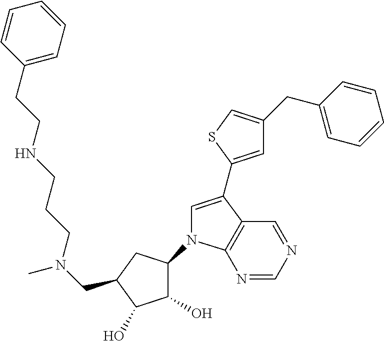 [SMILES: CN(CCCNCCc1ccccc1)C[C@H]1C[C@@H](n2cc(-c3cc(Cc4ccccc4)cs3)c3cncnc32)[C@H](O)[C@@H]1O]

To a solution of tert-butyl-N-[3-({[(1R,2R,3S,4R)-4-[5-(4-benzylthiophen-2-yl)pyrrolo[2,3-d]pyrimidin-7-yl]-2,3-dihydroxycyclopentyl]methyl}amino)propyl]-N-(2-phenylethyl)carbamate 248c (70 mg, 0.10 mmol, 1.0 eq) and AcOH (0.3 mg, 0.005 mmol, 0.05 eq) in DCM (2.5 mL) was added NaBH (OAc) 3 (32 mg, 0.15 mmol,1.5 eq) in several portions over 2 min, at 0° C. The reaction mixture was stirred for 2 h at 25° C. and the resulting mixture was concentrated under reduced pressure. The residue was dissolved in 4M HCl in MeOH (2.0 mL, 8.00 mmol) and the reaction mixture was stirred at 50° C. for 10 min. The resulting mixture was concentrated under reduced pressure, dissolved in MeOH (2.0 mL), adjusted to pH 8 with NH₃·H₂O and the crude residue was purified by Prep-HPLC [Column: Welch Ultimate XB-C18, 50*250 mm, 10 μm; Mobile Phase A: 0.1% HCl, Mobile Phase B: ACN; Flow rate: 90 mL/min; Gradient: 5% B-5% B-3 min, 10% B-50% B-12 min]: to afford (1R,2S,3R,5R)-3-[5-(4-benzylthiophen-2-yl)pyrrolo[2,3-d]pyrimidin-7-yl]-5-{[methyl({3-[(2-phenylethyl)amino]propyl})amino]methyl}cyclopentane-1,2-diol 620 (3.6 mg, 6%) as an off-white solid. LCMS [Cortecs C18+, 50*3.0 mm, 2.7 μm, Mobile Phase A: Water/0.02% TFA, Mobile Phase B: Acetonitrile, 5%-95% B-3 min, 1.50 L/min, ES, m/z]: T_{R}=1.38 min; [M+H]⁺: 596. ¹H NMR (400 MHZ, DMSO-d₆) δ 9.23 (s, 1H), 8.82 (s, 1H), 8.13 (s, 1H), 7.35 (s, 1H), 7.34-7.24 (m, 4H), 7.23-7.17 (m, 3H), 7.14-6.91 (m, 4H), 5.10-4.97 (m, 1H), 4.32 (dd, J=9.2, 5.5 Hz, 1H), 3.96 (s, 2H), 3.74 (d, J=4.9 Hz, 1H), 2.63-2.54 (m, 4H), 2.42-2.40 (m, 2H), 2.39-2.27 (m, 4H), 2.19-2.08 (m, 5H), 1.65-1.41 (m, 3H). Chiral-HPLC [Column: CHIRALPAK IH-3, 50*4.6 mm, 3 μm IH30CC-WH004; Mobile Phase A: MtBE (0.2% MSA), Mobile Phase B: Methanol (0.2% MSA); Total Flow: 1.00 mL/min; Conc. of Pump B: 30.0%; Oven Temperature: 25° C.]: T_{R}=1.19 min, single peak.

### Synthesis of (1R,2S,3R,5R)-3-[5-(4-benzylthiophen-2-yl)-2-chloropyrrolo[2,3-d]pyrimidin-7-yl]-5-[{{3-[(2-phenylethyl)amino]propyl}amino)methyl]cyclopentane-1,2-diol (621)

### tert-Butyl-N-{[(3aR,4R,6R,6aS)-6-{5-bromo-2-chloropyrrolo[2,3-d]pyrimidin-7-yl}-2,2-dimethyl-tetrahydro-3aH-cyclopenta[d][1,3]dioxol-4-yl]methyl}carbamate (249c)

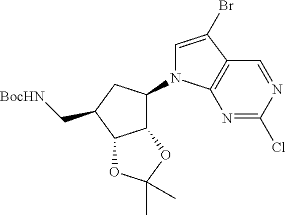 [SMILES: CC(C)(C)OC(=O)NC[C@H]1C[C@@H](n2cc(Br)c3cnc(Cl)nc32)[C@@H]2OC(C)(C)O[C@H]12]

To a solution of tert-butyl-N-{[(3aR,4R,6S,6aS)-6-hydroxy-2,2-dimethyl-tetrahydro-3aH-cyclopenta[d][1,3]dioxol-4-yl]methyl}carbamate 242c (0.45 g, 1.60 mmol, 1.2 eq) in toluene (3.0 mL) were added 5-bromo-2-chloro-7H-pyrrolo[2,3-d]pyrimidine (0.30 g, 1.30 mmol, 1.0 eq) and PPh₃ (0.68 g, 2.60 mmol, 2.0 eq) and the resulting mixture was stirred for 5 min at 0° C. To the above mixture was added, dropwise, over 5 min, at 0° C. under N₂, a solution of DBAD (601 mg, 2.61 mmol, 2.0 eq) in toluene (2.0 mL) and the reaction mixture was stirred at 60° C. for 15 h. The resulting mixture was concentrated under reduced pressure and the crude residue was purified by silica gel column chromatography (3:1 Petroleum ether: THF) to afford tert-butyl-N-{[(3aR,4R,6R,6aS)-6-{5-bromo-2-chloropyrrolo[2,3-d]pyrimidin-7-yl}-2,2-dimethyl-tetrahydro-3aH-cyclopenta[d][1,3]dioxol-4-yl]methyl}carbamate 249c (500 mg, 76%) as a yellow solid. LCMS [conditions: L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min (+), 1.50 L/min, ES, m/z]: T_{R}=2.10 min; [M+H]⁺: 501, 503.

### tert-Butyl-N-{[(3aR,4R,6R,6aS)-6-[5-(4-benzylthiophen-2-yl)-2-chloropyrrolo[2,3-d]pyrimidin-7-yl]-2,2-dimethyl-tetrahydro-3aH-cyclopenta[d][1,3]dioxol-4-yl]methyl}carbamate (250c)

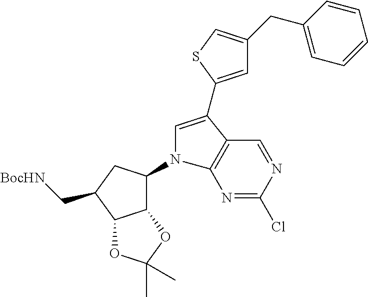 [SMILES: CC(C)(C)OC(=O)NC[C@H]1C[C@@H](n2cc(-c3cc(Cc4ccccc4)cs3)c3cnc(Cl)nc32)[C@@H]2OC(C)(C)O[C@H]12]

To a solution of tert-butyl-N-{[(3aR,4R,6R,6aS)-6-{5-bromo-2-chloropyrrolo[2,3-d]pyrimidin-7-yl}-2,2-dimethyl-tetrahydro-3aH-cyclopenta[d][1,3]dioxol-4-yl]methyl}carbamate 249c (300 mg, 0.60 mmol, 1.0 eq) in dioxane (3.0 mL) were added 2-(4-benzylthiophen-2-yl)-4,4,5,5-tetramethyl-1,3,2-dioxaborolane 315c (124 mg, 0.72 mmol, 1.2 eq), Pd (DtBPF) Cl₂ (77.9 mg, 0.12 mmol, 0.2 eq) and potassium phosphate tribasic (444 mg, 2.10 mmol, 3.5 eq) and the reaction mixture was stirred at 80° C. for 20 min, under N₂. The reaction mixture was concentrated under reduced pressure and the crude product was purified by flash chromatography (2:1 Petroleum ether: THF) to afford tert-butyl-N-{[(3aR,4R,6R,6aS)-6-[5-(4-benzylthiophen-2-yl)-2-chloropyrrolo[2,3-d]pyrimidin-7-yl]-2,2-dimethyl-tetrahydro-3aH-cyclopenta[d][1,3]dioxol-4-yl]methyl}carbamate 250c (292 mg, 82%) as a pale yellow oil. LCMS [conditions: Kinetex XB-C18,50*3.0 mm, 2.6 μm, Mobile Phase A: Water/0.05% TFA, Mobile Phase B: Acetonitrile, 2%-100% B-2 min (+).lcm]: T_{R}=1.28 min; [M+H]⁺: 595, 597.

### (1R,2S,3R,5R)-3-[5-(4-Benzylthiophen-2-yl)-2-chloropyrrolo[2,3-d]pyrimidin-7-yl]-5-[{{3-[(2-phenylethyl)amino]propyl}amino)methyl]cyclopentane-1,2-diol (251c)

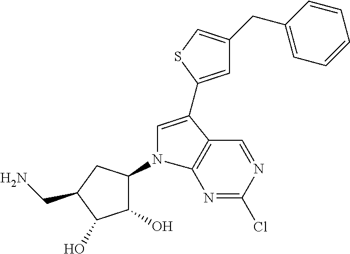 [SMILES: NC[C@H]1C[C@@H](n2cc(-c3cc(Cc4ccccc4)cs3)c3cnc(Cl)nc32)[C@H](O)[C@@H]1O]

To tert-butyl-N-{[(3aR,4R,6R,6aS)-6-[5-(4-benzylthiophen-2-yl)-2-chloropyrrolo[2,3-d]pyrimidin-7-yl]-2,2-dimethyl-tetrahydro-3aH-cyclopenta[d][1,3]dioxol-4-yl]methyl}carbamate 250c (120 mg, 0.20 mmol, 1.0 eq) was added 4M HCl in MeOH (1.0 mL, 4.0 mmol) and the reaction mixture was stirred at 50° C. for 1 h. The resulting mixture was concentrated under reduced pressure to afford (1R,2S,3R,5R)-3-[5-(4-benzylthiophen-2-yl)-2-chloropyrrolo[2,3-d]pyrimidin-7-yl]-5-[{{3-[(2-phenylethyl)amino]propyl}amino)methyl]cyclopentane-1,2-diol 251c (90 mg, crude) which was used in the next step without purification. LCMS [conditions: Cortecs C18+, 50*3.0 mm, 2.7 μm, Mobile Phase A: Water/0.05% TFA Mobile Phase B: Acetonitrile, 2%-95% B-3 min (+).Icm]: T_{R}=1.15 min; [M+H]⁺: 455, 457.

### tert-Butyl-N-[3-({[(1R,2R,3S,4R)-4-[5-(4-benzylthiophen-2-yl)-2-chloropyrrolo[2,3-d]pyrimidin-7-yl]-2,3-dihydroxycyclopentyl]methyl}amino)propyl]-N-(2-phenylethyl)carbamate (252c)

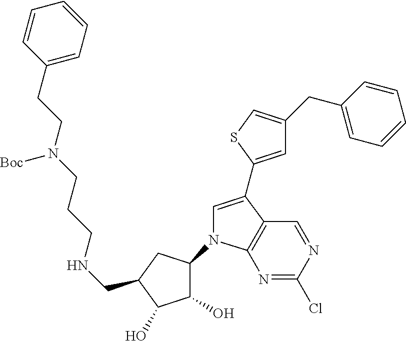 [SMILES: CC(C)(C)OC(=O)N(CCCNC[C@H]1C[C@@H](n2cc(-c3cc(Cc4ccccc4)cs3)c3cnc(Cl)nc32)[C@H](O)[C@@H]1O)CCc1ccccc1]

To a solution of (1S,2R,3R,5R)-3-(aminomethyl)-5-[5-(4-benzylthiophen-2-yl)-2-chloropyrrolo[2,3-d]pyrimidin-7-yl]cyclopentane-1,2-diol 251c (90 mg, 0.20 mmol, 1.0 eq), NaBH (OAc) 3 (84 mg, 0.40 mmol, 2.0 eq) and AcOH (119 mg, 2.0 mmol, 10 eq) in DCM (2.0 mL) was added a solution of tert-butyl-N-(3-oxopropyl)-N-(2-phenylethyl)carbamate 71c (60 mg, 0.22 mmol, 1.1 eq) in DCM (1.0 mL), at 0° C., under nitrogen, and the reaction mixture was stirred at 0° C. for 40 min. The resulting mixture was concentrated under reduced pressure to afford tert-butyl-N-[3-({[(1R,2R,3S,4R)-4-[5-(4-benzylthiophen-2-yl)-2-chloropyrrolo[2,3-d]pyrimidin-7-yl]-2,3-dihydroxycyclopentyl]methyl}amino)propyl]-N-(2-phenylethyl)carbamate 252 (50 mg, crude) as a green oil which was used in the next step directly without purification. LCMS [conditions: L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min (+).lcm 90-2000.lcm)]: T_{R}=2.35 min; [M+H]⁺: 716, 718.

### (1R,2S,3R,5R)-3-[5-(4-Benzylthiophen-2-yl)-2-chloropyrrolo[2,3-d]pyrimidin-7-yl]-5-[{{3-[(2-phenylethyl)amino]propyl}amino)methyl]cyclopentane-1,2-diol (621)

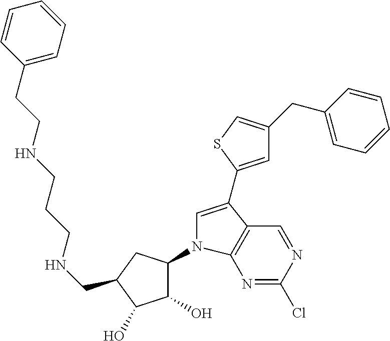 [SMILES: O[C@@H]1[C@@H](CNCCCNCCc2ccccc2)C[C@@H](n2cc(-c3cc(Cc4ccccc4)cs3)c3cnc(Cl)nc32)[C@@H]1O]

To tert-butyl-N-[3-({[(3aR,4R,6R,6aS)-2,2-dimethyl-6-[5-(thiophen-2-yl)pyrrolo[2,3-d]pyrimidin-7-yl]-tetrahydro-3aH-cyclopenta[d][1,3]dioxol-4-yl]methyl}(1H-pyrazol-4-yl)amino)propyl]-N-(2-phenylethyl)carbamate 252c (30 mg, crude) was added 4M HCl in MeOH (2.0 mL, 8.0 mmol) at room temperature and the reaction mixture was stirred at 50° C. for 10 min. The resulting mixture was concentrated under reduced pressure, dissolved with 2.0 mL MeOH, the solution adjusted to pH 8 with NH₃·H₂O and the crude residue was purified by Prep-HPLC [Column: Xbridge C18,19*150 mm, 5 μm; Mobile Phase A: Water (20 mmol/L NH₄HCO₃+0.05% NH₃·H₂O), Mobile Phase B: ACN; Flow rate: 30 ml/min; Gradient: 20% B to 45% B in 10 min; Wave Length: 254 nm]: to afford (1R,2S,3R,5R)-3-[5-(4-benzylthiophen-2-yl)-2-chloropyrrolo[2,3-d]pyrimidin-7-yl]-5-[{{3-[(2-phenylethyl)amino]propyl}amino)methyl]cyclopentane-1,2-diol 621 (11.2 mg, 22%) as an off-white solid. LCMS [conditions: ZORBAX SB-Aq, 50*4.6 mm, 1.8 μm; Mobile Phase A: Water/0.02% TFA, Mobile Phase B: Acetonitrile; 5%-95% B-3 min-1.5.lcm]: T_{R}=1.45 min; [M+H]⁺: 616, 618. ¹H NMR (400 MHZ, DMSO-d₆+D₂O) δ9.14 (s, 1H), 8.22 (s, 1H), 7.38 (s, 1H), 7.33-7.30 (m, 4H), 7.32-7.10 (m, 7H), 5.08-4.94 (m, 1H), 4.23 (dd, J=8.9, 5.4 Hz, 1H), 3.96 (s, 2H), 3.80 (br s, 1H), 2.90-2.40 (m, 10H), 2.36-2.15 (m, 1H), 2.11-2.01 (m, 1H), 1.58-1.52 (m, 3H).

### Synthesis of (1R,2S,3R,5R)-3-(2-chloro-5-(1-(methylsulfonyl)-1H-pyrazol-3-yl)-7H-pyrrolo[2,3-d]pyrimidin-7-yl)-5-(((3-(phenethylamino)propyl)amino)methyl)cyclopentane-1,2-diol (622)

### Benzyl-N-{[(3aR,4R,6aR)-2,2-dimethyl-6-oxo-tetrahydrocyclopenta[d][1,3]dioxol-4-yl]methyl}carbamate (253c)

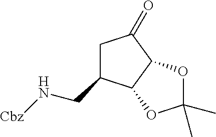 [SMILES: CC1(C)O[C@@H]2[C@@H](CNC(=O)OCc3ccccc3)CC(=O)[C@@H]2O1]

To a solution of (3aR,6aR)-2,2-dimethyl-3aH,6aH-cyclopenta[d][1,3]dioxol-4-one (3.00 g, 19.4 mmol, 1.0 eq) in DMF (30 mL) was added N-benzyloxycarbonylglycine (6.11 g, 29.2 mmol, 1.5 eq), IR [DF (CF₃) PPY]2 (DTBPY) PF₆ (109 mg, 1.0 mmol, 0.01 eq), K₂HPO₄ (6.78 g, 38.9 mmol, 2.0 eq) and the reaction mixture was stirred at room temperature for 24 h in blue LED (60 W), under N₂. The resulting mixture was diluted with water (50 mL) and extracted with AcOEt (3×50 mL). The combined organic layers were washed with brine, dried over anhydrous Na₂SO₄ and filtered. The filtrate was concentrated under reduced pressure and the crude residue was purified by silica gel column chromatography (3:1 Petroleum ether: THF) to afford benzyl-N-{[(3aR,4R,6aR)-2,2-dimethyl-6-oxo-tetrahydrocyclopenta[d][1,3]dioxol-4-yl]methyl}carbamate 253c (3.00 g, 48%) as pale yellow oil. LCMS [conditions L-column3 C18, 50*3.0 mm, 3.0 μm, Mobile Phase A: Water/5 mM NH₄HCO₃, Mobile Phase B: Methanol, 2%-100% B-3 min (+), 1.50 L/min, ES, m/z]: T_{R}=2.32 min; [M+H]⁺: 320.

### Benzyl-N-{[(3aR,4R,6S,6aS)-6-hydroxy-2,2-dimethyl-tetrahydro-3aH-cyclopenta[d][1,3]dioxol-4-yl]methyl}carbamate (254c)

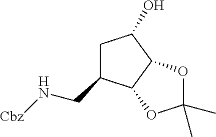 [SMILES: CC1(C)O[C@@H]2[C@@H](CNC(=O)OCc3ccccc3)C[C@H](O)[C@@H]2O1]

To a solution of benzyl-N-{[(3aR,4R,6aR)-2,2-dimethyl-6-oxo-tetrahydrocyclopenta[d][1,3]dioxol-4-yl]methyl}carbamate 253c (3.00 g, 9.40 mmol, 1.0 eq) in MeOH (30.0 mL) was added NaBH₄ (0.36 g, 9.40 mmol, 1.0 eq) in 5 equal portions and the reaction mixture was stirred for 30 min at room temperature. The resulting mixture was concentrated under reduced pressure and the crude residue was purified by silica gel column chromatography (3:2 Petroleum ether: THF) to afford benzyl-N-{[(3aR,4R,6S,6aS)-6-hydroxy-2,2-dimethyl-tetrahydro-3aH-cyclopenta[d][1,3]dioxol-4-yl]methyl}carbamate 254c (2.50 g, 82%) as colorless oil. LCMS [conditions Proshell HPH-C18, 50*3.0 mm, 2.7 μm, Mobile PhaseA: Water (0.05% ammonia water), Mobile Phase B: Acetonitrile, 2%-100% B-3 min (+,-), 1.50 L/min, ES, m/z]: T_{R}=1.72 min; [M+H]⁺: 322.

### (3aS,4S,6R,6aR)-6-(Aminomethyl)-2,2-dimethyl-tetrahydro-3aH-cyclopenta[d][1,3]dioxol-4-ol (255c)

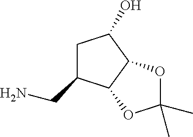

To a solution of benzyl-N-{[(3aR,4R,6S,6aS)-6-hydroxy-2,2-dimethyl-tetrahydro-3aH-cyclopenta[d][1,3]dioxol-4-yl]methyl}carbamate 254c (2.50 g, 7.80 mmol, 1.0 eq) in MeOH (25 mL) was added 10% Pd/C (0.69 g, 3.9 mmol, 0.5 eq) and the reaction mixture was stirred at 25° C. for 2 h under hydrogen. The resulting mixture was filtered, the filter cake was washed with MeOH (3×30 mL) and the filtrate was concentrated under reduced pressure to afford (3aS,4S,6R,6aR)-6-(aminomethyl)-2,2-dimethyl-tetrahydro-3aH-cyclopenta[d][1,3]dioxol-4-ol 255c (1.20 g, 82%) as colorless oil. LCMS [conditions Kinetex EVO C18, 50*3.0 mm, 2.6 μm, Mobile PhaseA: Water/5 mM NH₄HCO₃+0.05% Ammonia, Mobile Phase B: Acetonitrile/5% water, 2%-100% B-3 min (+), 1.50 L/min, ES, m/z]: T_{R}=0.91 min; [M+H]⁺: 188.

### tert-Butyl-N-[3-({[(3aR,4R,6S,6aS)-6-hydroxy-2,2-dimethyl-tetrahydro-3aH-cyclopenta[d][1,3]dioxol-4-yl]methyl}(tert-butoxycarbonyl)amino)propyl]-N-(2-phenylethyl)carbamate (256c)

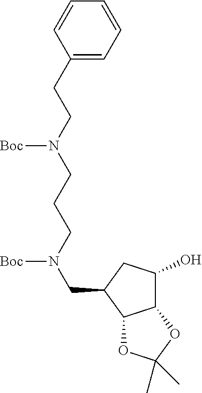 [SMILES: CC(C)(C)OC(=O)N(CCCN(C[C@H]1C[C@H](O)[C@@H]2OC(C)(C)O[C@H]12)C(=O)OC(C)(C)C)CCc1ccccc1]

To a solution of (3aS,4S,6R,6aR)-6-(aminomethyl)-2,2-dimethyl-tetrahydro-3aH-cyclopenta[d][1,3]dioxol-4-ol 255c (1.20 g, 6.40 mmol, 1.0 eq) in DCM (20 mL) was added tert-butyl N-(3-oxopropyl)-N-(2-phenylethyl)carbamate 71c (1.60 g, 5.80 mmol, 0.9 eq), AcOH (0.77 g, 12.8 mmol, 2.0 eq) and NaBH (OAc) 3 (2.71 g, 12.8 mmol, 2.0 eq) and the reaction mixture was stirred for 2 h at room temperature. Subsequently, TEA (1.95 g, 19.2 mmol, 3.0 eq) and Boc₂O (2.80 g, 12.8 mmol, 2.0 eq) were added and the reaction mixture was stirred for 1 h at 25° C. The reaction was quenched with water at 0° C. and the resulting mixture was concentrated under reduced pressure. The resulting residue was partitioned between AcOEt and brine, dried over anhydrous Na₂SO₄ and the filtrate was concentrated under reduced pressure. The crude residue was purified by silica gel column chromatography (3:2 Petroleum ether: THF) to afford tert-butyl-N-[3-({[(3aR,4R,6S,6aS)-6-hydroxy-2,2-dimethyl-tetrahydro-3aH-cyclopenta[d][1,3]dioxol-4-yl]methyl}(tert-butoxycarbonyl)amino)propyl]-N-(2-phenylethyl)carbamate 256c (3.00 g, 85%) as colorless oil. LCMS [Conditions L-column3 ODS, 50*3.0 mm, 3.0 μm, Mobile PhaseA: Water/5 mM NH₄HCO₃+0.05% Ammonia, Mobile Phase B: Acetonitrile/5% water, 2%-100% B-3 min (+), 1.50 L/min, ES, m/z]: T_{R}=1.85 min; [M+H]⁺: 549.

### 2-Chloro-5-(1-(methylsulfonyl)-1H-pyrazol-3-yl)-7H-pyrrolo[2,3-d]pyrimidine (257c)

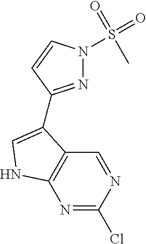 [SMILES: CS(=O)(=O)n1ccc(-c2c[nH]c3nc(Cl)ncc23)n1]

To a solution of 5-bromo-2-chloro-7H-pyrrolo[2,3-d]pyrimidine (230 mg, 0.10 mmol, 1.0 eq) and 1-methanesulfonyl-3-(4,4,5,5-tetramethyl-1,3,2-dioxaborolan-2-yl)pyrazole 200c (323 mg, 1.19 mmol, 1.2 eq) in 1,4-dioxane (3.0 mL) and H₂O (0.3 mL) was added Pd (DtBPF)Cl₂ (129 mg, 0.20 mmol, 0.2 eq) and K₃PO₄ (630 mg, 3.00 mmol, 3.0 eq) and the reaction mixture was stirred at 90° C. for 1 h, under nitrogen. The resulting mixture was concentrated under reduced pressure and the residue was purified by silica gel column chromatography (1:1 Petroleum ether: AcOEt) to afford 3-{2-chloro-7H-pyrrolo[2,3-d]pyrimidin-5-yl}-1-methanesulfonylpyrazole 257c (150 mg, 51%) as a yellow solid. LCMS [column: XBridge C18, 50*3.0 mm, 3.5 μm, Mobile Phase A: Water/5 mM NH₄HCO₃, Mobile Phase B: Methanol, 10%-95% B-3 min, 1.00 mL/min, ES, m/z]: T_{R}=1.30 min; [M+H]⁺: 298, 300.

### tert-Butyl-(3-((tert-butoxycarbonyl)(((3aR,4R,6R,6aS)-6-(2-chloro-5-(1-(methylsulfonyl)-1H-pyrazol-3-yl)-7H-pyrrolo[2,3-d]pyrimidin-7-yl)-2,2-dimethyltetrahydro-4H-cyclopenta[d][1,3]dioxol-4-yl)methyl)amino)propyl)(phenethyl)carbamate (258c)

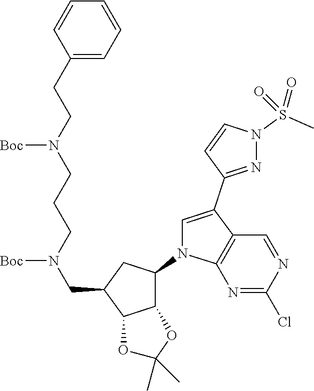 [SMILES: CC(C)(C)OC(=O)N(CCCN(C[C@H]1C[C@@H](n2cc(-c3ccn(S(C)(=O)=O)n3)c3cnc(Cl)nc32)[C@@H]2OC(C)(C)O[C@H]12)C(=O)OC(C)(C)C)CCc1ccccc1]

To a solution of 3-{2-chloro-7H-pyrrolo[2,3-d]pyrimidin-5-yl}-1-methanesulfonylpyrazole 257c (150 mg, 0.50 mmol, 1.0 eq) and tert-butyl-N-[3-({[(3aR,4R,6S,6aS)-6-hydroxy-2,2-dimethyl-tetrahydro-3aH-cyclopenta[d][1,3]dioxol-4-yl]methyl}(tert-butoxycarbonyl)amino)propyl]-N-(2-phenylethyl)carbamate 256c (414 mg, 0.76 mmol, 1.5 eq) in toluene (4.0 mL) were added DBAD (236 mg, 1.00 mmol, 2.0 eq), PPh₃ (303 mg, 1.16 mmol, 2.3 eq), at room temperature and the reaction mixture was stirred at 50° C. for 1 h, under nitrogen. The resulting mixture was concentrated under reduced pressure and the crude residue was purified by silica gel column chromatography (1:1 Petroleum ether: AcOEt) to afford tert-butyl-N-[3-({[(3aR,4R,6R,6aS)-6-[2-chloro-5-(1-methanesulfonylpyrazol-3-yl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate 258c (70 mg, 16%) as a yellow solid. LCMS [column: L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min, 1.20 mL/min, ES, m/z]: T_{R}=2.47 min; [M+H]⁺: 828, 830.

### (1R,2S,3R,5R)-3-(2-Chloro-5-(1-(methylsulfonyl)-1H-pyrazol-3-yl)-7H-pyrrolo[2,3-d]pyrimidin-7-yl)-5-(((3-(phenethylamino)propyl)amino)methyl)cyclopentane-1,2-diol (622)

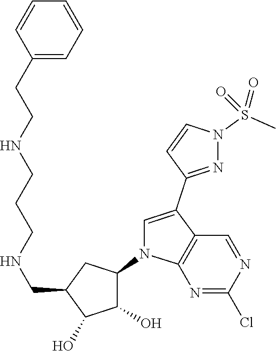 [SMILES: CS(=O)(=O)n1ccc(-c2cn([C@@H]3C[C@H](CNCCCNCCc4ccccc4)[C@@H](O)[C@H]3O)c3nc(Cl)ncc23)n1]

To tert-butyl-N-[3-({[(3aR,4R,6R,6aS)-6-[2-chloro-5-(1-methanesulfonylpyrazol-3-yl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate 258c (70 mg, 0.08 mmol, 1.0 eq) was added 4M HCl in MeOH (2.0 mL, 8.0 mmol) at room temperature and the reaction mixture was stirred at 50° C. for 1 h. The resulting mixture was concentrated under reduced pressure, adjusted to pH 8 with NH₃·H₂O and the crude residue was purified by Prep-HPLC [Column, XBridge Shield RP18 OBD Column, 19*150 mm, 5 μm; mobile phase, Water (10 mmol/L NH₄HCO₃+0.1% NH₃·H₂O) and ACN (18% ACN up to 68% in 7.5 min); Detector, UV 254 nm]: to afford (1R,2S,3R,5R)-3-[2-chloro-5-(1-methanesulfonylpyrazol-3-yl)pyrrolo[2,3-d]pyrimidin-7-yl]-5-[({3-[(2-phenylethyl)amino]propyl}amino)methyl]cyclopentane-1,2-diol 622 (16.8 mg, 34%) as an off-white solid. LCMS [column: ZORBAX SB-Aq, 50*4.6 mm, 1.8 μm. mobile phase A: Water/0.02% TFA, mobile phase B: Acetonitrile, 5%-95% B-3 min, 1.50 mL/min, ES, m/z]: T_{R}=1.24 min; [M+H]⁺: 588, 590. ¹H NMR (400 MHZ, DMSO-d₆) δ 9.35 (s, 1H), 8.53 (s, 1H), 8.36 (d, J=2.8 Hz, 1H), 7.28-7.23 (m, 2H), 7.22-7.11 (m, 3H), 7.10-7.07 (m, 1H), 5.07-4.88 (m, 2H), 4.27-4.22 (m, 1H), 3.84-3.77 (m, 1H), 3.64 (s, 3H), 2.72-2.67 (m, 5H), 2.60-2.55 (m, 5H), 2.33-2.21 (m, 1H), 2.11-2.06 (m, 1H), 1.63-1.50 (m, 3H). Chiral-HPLC [Column Name: CHIRALPAK IA-3, 50*4.6 mm, 3 μm IA30CC-WE011 Co Solvent: B: Methanol (0.2% MSA), Start Conc. of Pump B: 20.0%; Oven Temperature: 25° C.; Total Flow: 1.00 mL/min]: T_{R}=1.62 min (6 min run), single peak.

### Synthesis of (1R,2S,3R,5R)-3-(2-chloro-5-(1-(phenylsulfonyl)-1H-pyrazol-3-yl)-7H-pyrrolo[2,3-d]pyrimidin-7-yl)-5-(((3-(phenethylamino)propyl)amino)methyl)cyclopentane-1,2-diol (623)

### 2-Chloro-5-(1-(phenylsulfonyl)-1H-pyrazol-3-yl)-7H-pyrrolo[2,3-d]pyrimidine (259c)

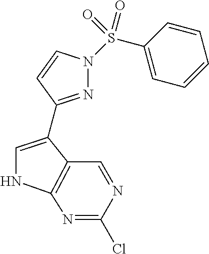 [SMILES: O=S(=O)(c1ccccc1)n1ccc(-c2c[nH]c3nc(Cl)ncc23)n1]

To a solution of 1-(benzenesulfonyl)pyrazol-3-ylboronic acid 204c (250 mg, 0.99 mmol, 1.0 eq) and 5-bromo-2-chloro-7H-pyrrolo[2,3-d]pyrimidine (276 mg, 1.20 mmol, 1.2 eq) in 1,4-dioxane (2.0 mL) and H₂O (0.2 mL) were added K₂CO₃ (411 mg, 2.97 mmol, 3.0 eq) and Pd (DtBPF) Cl₂ (129 mg, 0.20 mmol, 0.2 eq) and the reaction mixture was stirred at 100° C. for 1 h, under nitrogen. The resulting mixture was concentrated under reduced pressure and the crude residue was purified by silica gel column chromatography (1:1 Petroleum ether: AcOEt) to afford 1-(benzenesulfonyl)-3-{2-chloro-7H-pyrrolo[2,3-d]pyrimidin-5-yl}pyrazole 259c (90 mg, 25%) as a yellow solid. LCMS [column: L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min, 1.20 mL/min, ES, m/z]: T_{R}=1.85 min; [M+H]⁺: 360, 362.

### tert-Butyl-(3-((tert-butoxycarbonyl)(phenethyl)amino)propyl)(((3aR,4R,6R,6aS)-6-(2-chloro-5-(1-(phenylsulfonyl)-1H-pyrazol-3-yl)-7H-pyrrolo[2,3-d]pyrimidin-7-yl)-2,2-dimethyltetrahydro-4H-cyclopenta[d][1,3]dioxol-4-yl)methyl)carbamate (260c)

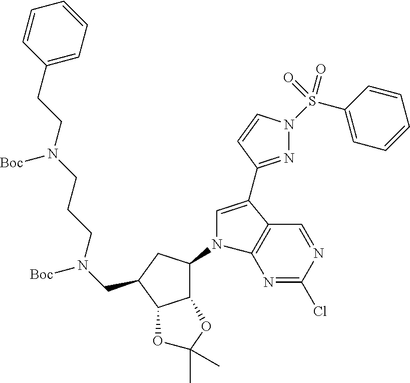 [SMILES: CC(C)(C)OC(=O)N(CCCN(C[C@H]1C[C@@H](n2cc(-c3ccn(S(=O)(=O)c4ccccc4)n3)c3cnc(Cl)nc32)[C@@H]2OC(C)(C)O[C@H]12)C(=O)OC(C)(C)C)CCc1ccccc1]

To a solution of 1-(benzenesulfonyl)-3-{2-chloro-7H-pyrrolo[2,3-d]pyrimidin-5-yl}pyrazole 259c (60 mg, 0.17 mmol, 1.0 eq) and tert-butyl-N-[3-({[(3aR,4R,6S,6aS)-6-hydroxy-2,2-dimethyl-tetrahydro-3aH-cyclopenta[d][1,3]dioxol-4-yl]methyl}(tert-butoxycarbonyl)amino)propyl]-N-(2-phenylethyl)carbamate 256c (137 mg, 0.25 mmol, 1.5 eq) in toluene (2.0 mL) were added DBAD (76 mg, 0.33 mmol, 2.0 eq) and PPh₃ (109 mg, 0.42 mmol, 2.5 eq) at room temperature and the reaction mixture was stirred at 60° C. for 1 h, under nitrogen. The resulting mixture was concentrated under reduced pressure and the crude residue was purified by silica gel column chromatography (1:1 Petroleum ether: AcOEt) to afford tert-butyl-N-[3-({[(3aR,4R,6R,6aS)-6-{5-[1-(benzenesulfonyl)pyrazol-3-yl]-2-chloropyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N(2-phenylethyl)carbamate 260c (25 mg, 17%) as a yellow solid. LCMS [column: L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min, 1.20 mL/min, ES, m/z]: T_{R}=2.49 min; [M+H]⁺: 890, 892.

### (1R,2S,3R,5R)-3-(2-Chloro-5-(1-(phenylsulfonyl)-1H-pyrazol-3-yl)-7H-pyrrolo[2,3-d]pyrimidin-7-yl)-5-(((3-(phenethylamino)propyl)amino)methyl)cyclopentane-1,2-diol (623)

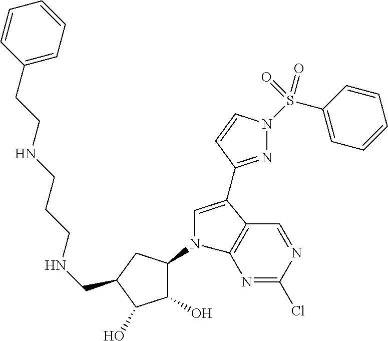 [SMILES: O=S(=O)(c1ccccc1)n1ccc(-c2cn([C@@H]3C[C@H](CNCCCNCCc4ccccc4)[C@@H](O)[C@H]3O)c3nc(Cl)ncc23)n1]

To tert-butyl-N-[3-({[(3aR,4R,6R,6aS)-6-{5-[1-(benzenesulfonyl)pyrazol-3-yl]-2-chloropyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 260c (17 mg, 0.02 mmol, 1.0 eq) was added TFA (2.0 mL) at room temperature and the reaction mixture was stirred at 50° C. for 1 h. The resulting mixture was concentrated under reduced pressure, adjusted to pH 8 with NH₃·H₂O and the crude residue was purified by Prep-HPLC [Column, XBridge Shield RP18 OBD Column, 19*150 mm, 5 μm; mobile phase, Water (10 mmol/L NH₄HCO₃+0.1% NH₃·H₂O) and ACN (20% ACN up to 72% in 8.5 min); Detector, UV 254 nm]: to afford (1R,2S,3R,5R)-3-{5-[1-(benzenesulfonyl)pyrazol-3-yl]-2-chloropyrrolo[2,3-d]pyrimidin-7-yl}-5-[({3-[(2-phenylethyl)amino]propyl}amino)methyl]cyclopentane-1,2-diol 623 (3.1 mg, 25%) as an off-white solid. LCMS (column: ZORBAX SB-Aq, 50*4.6 mm, 1.8 μm. mobile phase A: Water/0.02% TFA, mobile phase B: Acetonitrile, 5%-95% B-3 min, 1.50 mL/min, ES, m/z): T_{R}=1.35 min; [M+H]⁺: 650, 652. ¹H NMR (400 MHZ, DMSO-d₆) δ 9.20 (s, 1H), 8.56 (d, J=2.9 Hz, 1H), 8.48 (s, 1H), 8.09 (d, J=7.8 Hz, 2H), 7.79 (t, J=7.4 Hz, 1H), 7.69 (t, J=7.7 Hz, 2H), 7.27-7.19 (m, 2H), 7.19-7.07 (m, 4H), 5.02-4.88 (m, 2H), 4.73 (br. s, 1H), 4.23-4.17 (m, 1H), 3.81-3.75 (m, 1H), 2.69-2.64 (m, 5H), 2.60-2.54 (m, 5H), 2.30-2.18 (m, 1H), 2.08-2.04 (m, 1H), 1.58-1.46 (m, 3H). Chiral-HPLC [Column Name: CHIRALPAK IF-3, 100*4.6 mm, 3 μm IF30CS-UB006 Co Solvent: B: Ethanol (0.2% MAS), Start Conc. of Pump B: 50.0%; Oven Temperature: 25° C.; Total Flow: 1.00 mL/min]: T_{R}=4.10 min (12.5 min run), single peak.

### Synthesis of (1S,2R,3S,4R)-2,3-dihydroxy-N-methyl-4-(6,7,8,9-tetrahydro-2H-2,3,5,6-tetraazabenzo[cd]azulen-2-yl)cyclopentane-1-carboxamide (624)

### (3aR,4S,6R,6aS)-6-(5-Bromo-4-chloro-7H-pyrrolo[2,3-d]pyrimidin-7-yl)-N,2,2-trimethyltetrahydro-4H-cyclopenta[d][1,3]dioxole-4-carboxamide (261c)

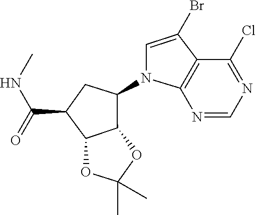 [SMILES: CNC(=O)[C@H]1C[C@@H](n2cc(Br)c3c(Cl)ncnc32)[C@@H]2OC(C)(C)O[C@@H]21]

To (3aR,4S,6R,6aS)-6-(5-bromo-4-chloro-7H-pyrrolo[2,3-d]pyrimidin-7-yl)-2,2-dimethyltetrahydro-4H-cyclopenta[d][1,3]dioxole-4-carboxylic acid 24c (220 mg, 0.53 mmol, 1.0 eq) in DMF (3.0 mL) were added HATU (242 mg, 0.64 mmol, 1.2 eq) and DIEA (205 mg, 1.59 mmol, 3.0 eq) and the resulting mixture was stirred for 10 min at room temperature. Methylamine (20 mg, 0.64 mmol, 1.2 eq) was then added and the reaction mixture was stirred for 1 h at room temperature. The resulting mixture was quenched with H₂O (10 mL) and extracted with AcOEt (3×10 mL). The combined organic layers were washed with brine, dried over anhydrous Na₂SO₄ and the filtrate was concentrated under reduced pressure. The crude residue was purified by silica gel column chromatography (1:3 AcOEt: petroleum ether) to afford (3aR,4S,6R,6aS)-6-(5-bromo-4-chloro-7H-pyrrolo[2,3-d]pyrimidin-7-yl)-N,2,2-trimethyltetrahydro-4H-cyclopenta[d][1,3]dioxole-4-carboxamide 261c (160 mg, 71%) as a light yellow solid. ¹H NMR (300 MHz, Chloroform-d) δ 8.66 (s, 1H), 7.54 (s, 1H), 5.37-5.23 (m, 1H), 5.03-4.93 (m, 1H), 4.93-4.83 (m, 1H), 3.46-3.33 (m, 1H), 3.03 (s, 3H), 2.60 (t, J=9.2 Hz, 2H), 1.62 (s, 3H), 1.32 (s, 3H).

### (3aR,4S,6R,6aS)-6-(4-Amino-5-bromo-7H-pyrrolo[2,3-d]pyrimidin-7-yl)-N,2,2-trimethyltetrahydro-4H-cyclopenta[d][1,3]dioxole-4-carboxamide (262c)

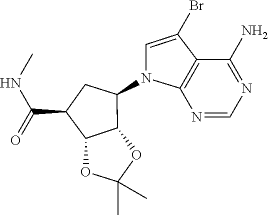 [SMILES: CNC(=O)[C@H]1C[C@@H](n2cc(Br)c3c(N)ncnc32)[C@@H]2OC(C)(C)O[C@@H]21]

To (3aR,4S,6R,6aS)-6-(5-bromo-4-chloro-7H-pyrrolo[2,3-d]pyrimidin-7-yl)-N,2,2-trimethyltetrahydro-4H-cyclopenta[d][1,3]dioxole-4-carboxamide 261c (100 mg, 0.23 mmol, 1.0 eq) was added 7M NH₃ in MeOH (2.0 mL, 14 mmol) and the reaction mixture was stirred at 75° C. for 8 h. The resulting mixture was concentrated under reduced pressure and the crude residue was purified by silica gel column chromatography (1:3 tetrahydrofuran: petroleum ether) to afford (3aR,4S,6R,6aS)-6-(4-amino-5-bromo-7H-pyrrolo[2,3-d]pyrimidin-7-yl)-N,2,2-trimethyltetrahydro-4H-cyclopenta[d][1,3]dioxole-4-carboxamide 262c (60 mg, 63%) as a white solid. ¹H NMR (300 MHZ, Chloroform-d) δ 8.29 (s, 1H), 7.20 (s, 1H), 5.88 (s, 2H), 5.30-5.19 (m, 1H), 5.03-4.93 (m, 1H), 4.90-4.79 (m, 1H), 3.42-3.29 (m, 1H), 3.02 (s, 3H), 2.64-2.42 (m, 2H), 1.60 (s, 3H), 1.32 (s, 3H).

### (3aR,4S,6R,6aS)-6-(4-Amino-5-(3,3-diethoxypropyl)-7H-pyrrolo[2,3-d]pyrimidin-7-yl)-N,2,2-trimethyltetrahydro-4H-cyclopenta[d][1,3]dioxole-4-carboxamide (263c)

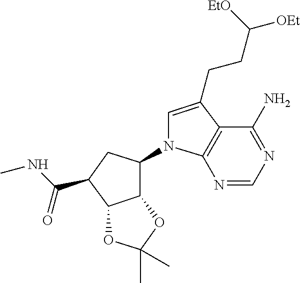 [SMILES: CCOC(CCc1cn([C@@H]2C[C@H](C(=O)NC)[C@H]3OC(C)(C)O[C@H]32)c2ncnc(N)c12)OCC]

To a solution of (3aR,4S,6R,6aS)-6-(4-amino-5-bromo-7H-pyrrolo[2,3-d]pyrimidin-7-yl)-N,2,2-trimethyltetrahydro-4H-cyclopenta[d][1,3]dioxole-4-carboxamide 262c (60 mg, 0.15 mmol, 1.0 eq), and (E)-2-(3,3-diethoxyprop-1-en-1-yl)-4,4,5,5-tetramethyl-1,3,2-dioxaborolane (28 mg, 0.22 mmol, 1.5 eq) in 1,4-dioxane (3.0 mL) and H₂O (0.6 mL) were added Pd (dtbpf) Cl₂ (5.0 mg, 0.008 mmol, 0.05 eq) and K₃PO₄ (96 mg, 0.45 mmol, 3.0 eq) and the reaction mixture was stirred at 90° C. for 1 h, under N₂. The resulting mixture was portioned between AcOEt and brine and the organic layer was dried over anhydrous Na₂SO₄. The filtrate was concentrated under reduced pressure and the crude residue was purified by silica gel column chromatography (1:1 AcOEt: petroleum ether) to afford (3aR,4S,6R,6aS)-6-(4-amino-5-(3,3-diethoxypropyl)-7H-pyrrolo[2,3-d]pyrimidin-7-yl)-N,2,2-trimethyltetrahydro-4H-cyclopenta[d][1,3]dioxole-4-carboxamide 263c (30 mg, 44%) as an off-white solid. LCMS [conditions ZORBAX SB-Aq, 50*4.6 mm, 1.8 μm, Mobile Phase A: Water/0.02% TFA, Mobile Phase B: Acetonitrile, 5%-95% B-3 min (+), 1.50 L/min, ES, m/z]: T_{R}=2.01 min; [M+H]⁺: 462.

### (3aR,4S,6R,6aS)-N,2,2-Trimethyl-6-(6,7,8,9-tetrahydro-2H-2,3,5,6-tetraazabenzo[cd]azulen-2-yl)tetrahydro-4H-cyclopenta[d][1,3]dioxole-4-carboxamide (264c)

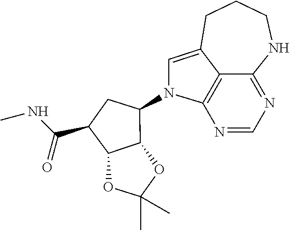 [SMILES: CNC(=O)[C@H]1C[C@@H](n2cc3c4c(ncnc42)NCCC3)[C@@H]2OC(C)(C)O[C@@H]21]

To (3aR,4S,6R,6aS)-6-(4-amino-5-(3,3-diethoxypropyl)-7H-pyrrolo[2,3-d]pyrimidin-7-yl)-N,2,2-trimethyltetrahydro-4H-cyclopenta[d][1,3]dioxole-4-carboxamide 263c (30 mg, 0.07 mmol, 1.0 eq) was added AcOH (90% aq., 1.5 mL) and the resulting mixture was stirred at 50° C. for 2 h. After allowing to cool to room temperature, 10% Pd/C (10 mg) was added and the reaction mixture was stirred for 2 h at room temperature, under H₂. The resulting mixture was filtered and the filtrate was concentrated under reduced pressure, adjusted to pH 8 with NH₃·H₂O and the crude mixture was purified by Prep-HPLC [Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 20% B to 65% B in 12 min, 65% B; Wave Length: 220 nm]: to afford (3aR,4S,6R,6aS)-N,2,2-trimethyl-6-(6,7,8,9-tetrahydro-2H-2,3,5,6-tetraazabenzo[cd]azulen-2-yl)tetrahydro-4H-cyclopenta[d][1,3]dioxole-4-carboxamide 264c (15 mg, 62%) as a pale yellow solid. LCMS [conditions ZORBAX SB-Aq, 50*4.6 mm, 1.8 μm, Mobile Phase A: Water/0.02% TFA, Mobile Phase B: Acetonitrile, 5%-95% B-3 min (+), 1.50 L/min, ES, m/z]: T_{R}=1.61 min; [M+H]⁺: 372.

### (1S,2R,3S,4R)-2,3-Dihydroxy-N-methyl-4-(6,7,8,9-tetrahydro-2H-2,3,5,6-tetraazabenzo[cd]azulen-2-yl)cyclopentane-1-carboxamide (624)

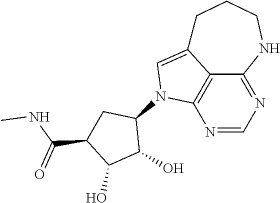 [SMILES: CNC(=O)[C@H]1C[C@@H](n2cc3c4c(ncnc42)NCCC3)[C@H](O)[C@@H]1O]

To (3aR,4S,6R,6aS)-N,2,2-trimethyl-6-(6,7,8,9-tetrahydro-2H-2,3,5,6-tetraazabenzo[cd]azulen-2-yl)tetrahydro-4H-cyclopenta[d][1,3]dioxole-4-carboxamide 264c (15 mg, 0.04 mmol, 1.0 eq) was added TFA (1.0 mL) and the reaction mixture was stirred at 50° C. for 30 min. The resulting mixture was concentrated under reduced pressure, adjusted to pH 8 with NH₃·H₂O and the crude residue was purified by Prep-HPLC [Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 20% B to 50% B in 10 min, 50% B; Wave Length: 220 nm]: to afford (1S,2R,3S,4R)-2,3-dihydroxy-N-methyl-4-(6,7,8,9-tetrahydro-2H-2,3,5,6-tetraazabenzo[cd]azulen-2-yl)cyclopentane-1-carboxamide 624 (3.1 mg, 23%) as a white solid. LCMS [conditions ZORBAX SB-Aq, 50*4.6 mm, 1.8 μm, Mobile Phase A: Water/0.02% TFA, Mobile Phase B: Acetonitrile, 5%-95% B-3 min (+), 1.50 L/min, ES, m/z]: T_{R}=1.01 min; [M+H]⁺: 332. ¹H NMR (300 MHZ, Methanol-d₄) δ 8.03 (s, 1H), 7.20 (s, 1H), 5.11-4.97 (m, 1H), 4.34-4.18 (m, 2H), 3.57-3.48 (m, 2H), 3.00-2.90 (m, 2H), 2.93-2.81 (m, 1H), 2.79 (s, 3H), 2.58-2.42 (m, 1H), 2.14-1.97 (m, 3H). Chiral-SFC [Column Name: SB 100×4.6 mm 3.0 μm, Solvent: B: MeOH (20 mM NH₃), Start Conc. of Pump B: 50.0%; Oven Temperature: 25° C.; Total Flow: 3.00 mL/min]: T_{R}=1.83 min (4 min run), single peak.

### Synthesis of (15,2R,3S,4R)-2,3-dihydroxy-N,N-dimethyl-4-(6,7,8,9-tetrahydro-2H-2,3,5,6-tetraazabenzo[cd]azulen-2-yl)cyclopentane-1-carboxamide (625)

### (3aR,4S,6R,6aS)-6-(5-Bromo-4-chloro-7H-pyrrolo[2,3-d]pyrimidin-7-yl)-N,N,2,2-tetramethyltetrahydro-4H-cyclopenta[d][1,3]dioxole-4-carboxamide (265c)

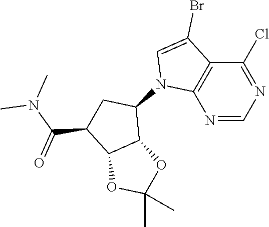 [SMILES: CN(C)C(=O)[C@H]1C[C@@H](n2cc(Br)c3c(Cl)ncnc32)[C@@H]2OC(C)(C)O[C@@H]21]

To (3aR,4S,6R,6aS)-6-(5-bromo-4-chloro-7H-pyrrolo[2,3-d]pyrimidin-7-yl)-2,2-dimethyltetrahydro-4H-cyclopenta[d][1,3]dioxole-4-carboxylic acid (220 mg, 0.53 mmol, 1.0 eq) in DMF (3.0 mL) were added DIEA (205 mg, 1.59 mmol, 3.0 eq) and HATU (242 mg, 0.64 mmol, 1.2 eq) and the resulting mixture was stirred for 10 min at room temperature. Dimethylamine (29 mg, 0.64 mmol, 1.2 eq) was then added and the reaction mixture was stirred for 1 h at room temperature. The resulting mixture was quenched with H₂O (10 mL) and extracted with AcOEt (3×10 mL). The combined organic layers were washed with brine, dried over anhydrous Na₂SO₄ and filtered. The filtrate was concentrated under reduced pressure and the crude residue was purified by silica gel column chromatography (1:3 AcOEt: petroleum ether) to afford (3aR,4S,6R,6aS)-6-(5-bromo-4-chloro-7H-pyrrolo[2,3-d]pyrimidin-7-yl)-N,N,2,2-tetramethyltetrahydro-4H-cyclopenta[d][1,3]dioxole-4-carboxamide 265c (170 mg, 73%) as a pale yellow solid. LCMS [conditions ZORBAX SB-Aq, 50*4.6 mm, 1.8 μm, Mobile Phase A: Water/0.02% TFA, Mobile Phase B: Acetonitrile, 5%-95% B-3 min (+), 1.50 L/min, ES, m/z]: T_{R}=1.81 min; [M+H]⁺: 443, 445.

### (3aR,45,6R,6aS)-6-(4-Amino-5-bromo-7H-pyrrolo[2,3-d]pyrimidin-7-yl)-N,N,2,2-tetramethyltetrahydro-4H-cyclopenta[d][1,3]dioxole-4-carboxamide (266c)

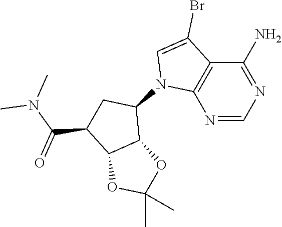 [SMILES: CN(C)C(=O)[C@H]1C[C@@H](n2cc(Br)c3c(N)ncnc32)[C@@H]2OC(C)(C)O[C@@H]21]

To (3aR,4S,6R,6aS)-6-(5-bromo-4-chloro-7H-pyrrolo[2,3-d]pyrimidin-7-yl)-N,N,2,2-tetramethyltetrahydro-4H-cyclopenta[d][1,3]dioxole-4-carboxamide 265c (170 mg, 0.38 mmol, 1.0 eq) was added 7M NH₃ in MeOH (2.5 mL, 17.5 mmol) and the reaction mixture was stirred at 75° C. for 8 h. The resulting mixture was concentrated under reduced pressure and the crude residue was purified by silica gel column chromatography (1:3 tetrahydrofuran: petroleum ether) to afford (3aR,4S,6R,6aS)-6-(4-amino-5-bromo-7H-pyrrolo[2,3-d]pyrimidin-7-yl)-N, N,2,2-tetramethyltetrahydro-4H-cyclopenta[d][1,3]dioxole-4-carboxamide 266c (140 mg, 86%) as a white solid. LCMS [conditions ZORBAX SB-Aq, 50*4.6 mm, 1.8 μm, Mobile Phase A: Water/0.02% TFA, Mobile Phase B: Acetonitrile, 5%-95% B-3 min (+), 1.50 L/min, ES, m/z]: T_{R}=1.54 min; [M+H]⁺: 424, 426.

### (3aR,4S,6R,6aS)-6-(4-Amino-5-(3,3-diethoxypropyl)-7H-pyrrolo[2,3-d]pyrimidin-7-yl)-N,N,2,2-tetramethyltetrahydro-4H-cyclopenta[d][1,3]dioxole-4-carboxamide (267c)

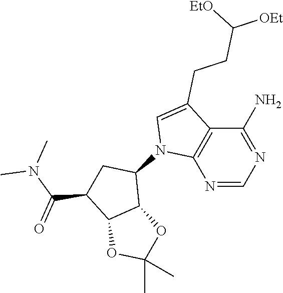 [SMILES: CCOC(CCc1cn([C@@H]2C[C@H](C(=O)N(C)C)[C@H]3OC(C)(C)O[C@H]32)c2ncnc(N)c12)OCC]

To a solution of (3aR,4S,6R,6aS)-6-(4-amino-5-bromo-7H-pyrrolo[2,3-d]pyrimidin-7-yl)-N,N,2,2-tetramethyltetrahydro-4H-cyclopenta[d][1,3]dioxole-4-carboxamide 266c (140 mg, 0.33 mmol, 1.0 eq), and (E)-2-(3,3-diethoxyprop-1-en-1-yl)-4,4,5,5-tetramethyl-1,3,2-dioxaborolane (64 mg, 0.49 mmol, 1.5 eq) in 1,4-dioxane (3.0 mL) and H₂O (0.6 mL), were added Pd (dtbpf) Cl₂ (10 mg, 0.016 mmol, 0.05 eq) and K₃PO₄ (210 mg, 0.99 mmol, 3.0 eq) and the reaction mixture was stirred at 90° C. for 1 h, under N₂. The resulting mixture was partitioned between AcOEt and brine and the organic layer was dried over anhydrous Na₂SO₄. The filtrate was concentrated under reduced pressure and the crude residue was purified by silica gel column chromatography (1:1 AcOEt: petroleum ether) to afford (3aR,4S,6R,6aS)-6-(4-amino-5-(3,3-diethoxypropyl)-7H-pyrrolo[2,3-d]pyrimidin-7-yl)-N,N,2,2-tetramethyltetrahydro-4H-cyclopenta[d][1,3]dioxole-4-carboxamide 267c (70 mg, 45%) as an off-white solid. LCMS [conditions ZORBAX SB-Aq, 50*4.6 mm, 1.8 μm, Mobile Phase A: Water/0.02% TFA, Mobile Phase B: Acetonitrile, 5%-95% B-3 min (+), 1.50 L/min, ES, m/z]: T_{R}=1.81 min; [M+H]⁺: 476.

### (3aR,4S,6R,6aS)-N,N,2,2-Tetramethyl-6-(6,7,8,9-tetrahydro-2H-2,3,5,6-tetraazabenzo[cd]azulen-2-yl)tetrahydro-4H-cyclopenta[d][1,3]dioxole-4-carboxamide (268c)

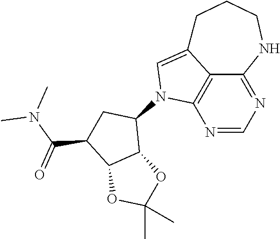 [SMILES: CN(C)C(=O)[C@H]1C[C@@H](n2cc3c4c(ncnc42)NCCC3)[C@@H]2OC(C)(C)O[C@@H]21]

To (3aR,4S,6R,6aS)-6-(4-amino-5-(3,3-diethoxypropyl)-7H-pyrrolo[2,3-d]pyrimidin-7-yl)-N, N,2,2-tetramethyltetrahydro-4H-cyclopenta[d][1,3]dioxole-4-carboxamide 267c (70 mg, 0.15 mmol, 1.0 eq) was added AcOH (90% aq., 2.0 mL) and the reaction mixture was stirred at 50° C. for 2 h. After allowing to cool to room temperature, 10% Pd/C (15 mg) was added and the reaction mixture stirred for 2 h at room temperature, under H₂. The resulting mixture was filtered, the filtrate was concentrated under reduced pressure, adjusted to pH 8 with NH₃·H₂O and the crude mixture was purified by Prep-HPLC [Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 20% B to 65% B in 12 min, 65% B; Wave Length: 220 nm]: to afford (3aR,4S,6R,6aS)-N,N,2,2-tetramethyl-6-(6,7,8,9-tetrahydro-2H-2,3,5,6-tetraazabenzo[cd]azulen-2-yl)tetrahydro-4H-cyclopenta[d][1,3]dioxole-4-carboxamide 268c (40 mg, 71%) as a pale yellow solid. LCMS [conditions ZORBAX SB-Aq, 50*4.6 mm, 1.8 μm, Mobile Phase A: Water/0.02% TFA, Mobile Phase B: Acetonitrile, 5%-95% B-3 min (+), 1.50 L/min, ES, m/z]: T_{R}=1.41 min; [M+H]⁺: 386.

### (1S,2R,3S,4R)-2,3-Dihydroxy-N,N-dimethyl-4-(6,7,8,9-tetrahydro-2H-2,3,5,6-tetraazabenzo[cd]azulen-2-yl)cyclopentane-1-carboxamide (625)

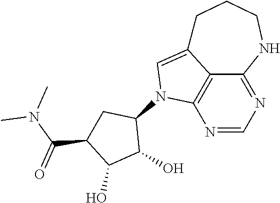 [SMILES: CN(C)C(=O)[C@H]1C[C@@H](n2cc3c4c(ncnc42)NCCC3)[C@H](O)[C@@H]1O]

To (3aR,4S,6R,6aS)-N,N,2,2-tetramethyl-6-(6,7,8,9-tetrahydro-2H-2,3,5,6-tetraazabenzo[cd]azulen-2-yl)tetrahydro-4H-cyclopenta[d][1,3]dioxole-4-carboxamide 268c (40 mg, 0.11 mmol, 1.0 eq) was added TFA (2.0 mL) and the reaction mixture was stirred at 50° C. for 30 min. The resulting mixture was concentrated under reduced pressure, adjusted to pH 8 with NH₃·H₂O and the crude residue was purified by Prep-HPLC [Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 20% B to 55% B in 10 min, 55% B; Wave Length: 220 nm]: to afford (1S,2R,3S,4R)-2,3-dihydroxy-N,N-dimethyl-4-(6,7,8,9-tetrahydro-2H-2,3,5,6-tetraazabenzo[cd]azulen-2-yl)cyclopentane-1-carboxamide 625 (11.5 mg, 32%) as a white solid. LCMS [conditions ZORBAX SB-Aq, 50*4.6 mm, 1.8 μm, Mobile Phase A: Water/0.02% TFA, Mobile Phase B: Acetonitrile, 5%-95% B-3 min (+), 1.50 L/min, ES, m/z]: T_{R}=1.06 min; [M+H]⁺: 346. 1H NMR (300 MHZ, Methanol-d₄) δ 8.03 (s, 1H), 7.19 (s, 1H), 5.18-5.03 (m, 1H), 4.31-4.22 (m, 2H), 3.57-3.48 (m, 2H), 3.46-3.33 (m, 1H), 3.20 (s, 3H), 3.02 (s, 3H), 2.99-2.89 (m, 2H), 2.61-2.45 (m, 1H), 2.18-1.96 (m, 3H). Chiral-SFC [Column Name: SB 100×4.6 mm 3.0 μm, Solvent: B: MeOH (20 mM NH₃), Start Conc. of Pump B: 50.0%; Oven Temperature: 25° C.; Total Flow: 3.00 mL/min]: T_{R}=0.97 min (4 min run), single peak.

### Synthesis of (2-((1R,2S,3R,4R)-4-(Aminomethyl)-2,3-dihydroxycyclopentyl)-2,6,8,9-tetrahydro-7H-2,3,5,6-tetraazabenzo[cd]azulen-7-one (626)

### 7-[(3aS,4R,6R,6aR)-6-(Azidomethyl)-2,2-dimethyl-tetrahydro-3aH-cyclopenta[d][1,3]dioxol-4-yl]-4-chloropyrrolo[2,3-d]pyrimidine (277c)

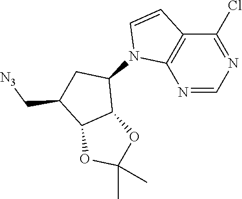 [SMILES: CC1(C)O[C@@H]2[C@@H](CN=[N+]=[N-])C[C@@H](n3ccc4c(Cl)ncnc43)[C@@H]2O1]

To [(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]methanol (2.00 g, 6.10 mmol, 1.0 eq) in THF (16 mL) was added PPh₃ (2.20 g, 8.60 mmol, 1.4 eq), under nitrogen, and the resultant mixture was cooled to −10° C. To this mixture was added DIAD (1.70 g, 8.60 mmol, 1.4 eq), dropwise over 5 min, and the reaction mixture was stirred at −10° C. for 10 min. To the above mixture was added a solution of DPPA (2.40 g, 8.60 mmol, 1.4 eq) in THF (4.0 mL), dropwise over 10 min, at −10° C. The reaction mixture was allowed to warm to room temperature and was then stirred for 3 h. The resulting mixture was concentrated under reduced pressure, dissolved in DCM (3.0 mL) and was then purified by silica gel column chromatography (7:3 petroleum ether/AcOEt) to afford 7-[(3aS,4R,6R,6aR)-6-(azidomethyl)-2,2-dimethyl-tetrahydro-3aH-cyclopenta[d][1,3]dioxol-4-yl]-4-chloropyrrolo[2,3-d]pyrimidine 277c (1.10 g, 51%) as a colorless oil. LCMS [conditions XBridge C18, 50*3.0 mm, 3.5 μm, Mobile Phase A: Water/5 mM NH₄HCO₃, Mobile Phase B: Methanol, 10%-95% B-3 min (+), 1.50 L/min, ES, m/z]: T_{R}=2.11 min; [M+H]⁺: 349, 351

### 1-[(3aR,4R,6R,6aS)-6-{4-Chloropyrrolo[2,3-d]pyrimidin-7-yl}-2,2-dimethyl-tetrahydro-3aH-cyclopenta[d][1,3]dioxol-4-yl]methanamine (278c)

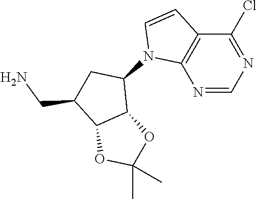 [SMILES: CC1(C)O[C@@H]2[C@@H](CN)C[C@@H](n3ccc4c(Cl)ncnc43)[C@@H]2O1]

To a solution of 7-[(3aS,4R,6R,6aR)-6-(azidomethyl)-2,2-dimethyl-tetrahydro-3aH-cyclopenta[d][1,3]dioxol-4-yl]-4-chloropyrrolo[2,3-d]pyrimidine 277c (1.10 g, 3.10 mmol, 1.0 eq) in THF (6.6 mL) was added PPh₃ (1.60 g, 6.30 mmol, 2.0 eq) and H₂O (4.4 mL) and the reaction mixture was stirred at 40° C. for 2 h, under N₂. The crude product 1-[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]methanamine 278c. was used in the next step directly without further purification. LCMS [conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min (+), 1.50 L/min, ES, m/z]: T_{R}=1.43; [M+H]⁺: 323, 325.

### tert-Butyl-N-{[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]methyl}carbamate (279c)

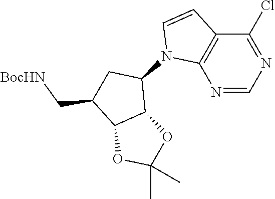 [SMILES: CC(C)(C)OC(=O)NC[C@H]1C[C@@H](n2ccc3c(Cl)ncnc32)[C@@H]2OC(C)(C)O[C@H]12]

To a solution of 1-[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]methanamine 278c (crude) in DCM (8.0 mL) and H₂O (4.0 mL) were added di-tert-butyl dicarbonate (1.50 g, 6.80 mmol, 2.0 eq) and TEA (1.0 g, 10.2 mmol, 3.0 eq) and the reaction mixture was stirred for 30 min at room temperature. The resulting mixture was extracted with AcOEt (3×20 mL) and the combined organic layers were washed with brine and dried over anhydrous Na₂SO₄. The filtrate was concentrated under reduced pressure and the crude residue was purified by silica gel column chromatography (96:4 DCM: MeOH) to afford tert-butyl-N-{[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]methyl}carbamate 279c (1.00 g, 69%) as a light brown oil. LCMS [conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min, 1.50 L/min, ES, m/z]: T_{R}=2.05 min; [M+H]⁺: 423, 425.

### tert-Butyl-N-{[(3aR,4R,6R,6aS)-6-{4-chloro-5-iodopyrrolo[2,3-d]pyrimidin-7-yl}-2,2-dimethyl-tetrahydro-3aH-cyclopenta[d][1,3]dioxol-4-yl]methyl}carbamate (280c)

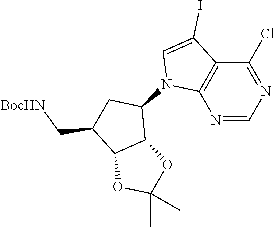 [SMILES: CC(C)(C)OC(=O)NC[C@H]1C[C@@H](n2cc(I)c3c(Cl)ncnc32)[C@@H]2OC(C)(C)O[C@H]12]

To a solution of tert-butyl-N-{[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]methyl}carbamate 279c (1.00 g, 2.40 mmol, 1.0 eq) in DMF (10 mL) was added a solution of NIS (0.80 g, 3.50 mmol, 1.5 eq) in DMF (2.0 mL), dropwise over 10 min, at 0° C., and the reaction mixture was stirred for 16 h at room temperature. The reaction was quenched with saturated Na₂S203 aq. at 0° C. and the resulting mixture was extracted with AcOEt (3×10.0 mL). The combined organic layers were washed with saturated NaHCO₃ aq., brine and dried over anhydrous Na₂SO₄. The filtrate was concentrated under reduced pressure and the crude residue was purified by silica gel column chromatography (1:3 AcOEt: petroleum ether) to afford tert-butyl-N-{[(3aR,4R,6R,6aS)-6-{4-chloro-5-iodopyrrolo[2,3-d]pyrimidin-7-yl}-2,2-dimethyl-tetrahydro-3aH-cyclopenta[d][1,3]dioxol-4-yl]methyl}carbamate 280c (1.10 g, 85%) as an off-white solid. LCMS [conditions XBridge C18, 50*3.0 mm, 3.5 μm, Mobile Phase A: Water/5 mM NH₄HCO₃, Mobile Phase B: Methanol, 10%-95% B-3 min (+), 1.50 L/min, ES, m/z]: T_{R}=2.29 min; [M+H]⁺: 549, 551.

### tert-Butyl-(((3aR,4R,6R,6aS)-6-(4-amino-5-iodo-7H-pyrrolo[2,3-d]pyrimidin-7-yl)-2,2-dimethyltetrahydro-4H-cyclopenta[d][1,3]dioxol-4-yl)methyl)carbamate (281c)

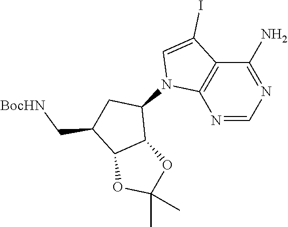 [SMILES: CC(C)(C)OC(=O)NC[C@H]1C[C@@H](n2cc(I)c3c(N)ncnc32)[C@@H]2OC(C)(C)O[C@H]12]

To tert-butyl-N-{[(3aR,4R,6R,6aS)-6-{4-chloro-5-iodopyrrolo[2,3-d]pyrimidin-7-yl}-2,2-dimethyl-tetrahydro-3aH-cyclopenta[d][1,3]dioxol-4-yl]methyl}carbamate 280c (0.50 g, 0.90 mmol, 1.0 eq) was added 7M NH₃ in MeOH (5.0 mL) at room temperature and the reaction mixture was stirred at 80° C. for 12 h. The resulting mixture was concentrated under reduced pressure to afford tert-butyl-N-{[(3aR,4R,6R,6aS)-6-{4-amino-5-iodopyrrolo[2,3-d]pyrimidin-7-yl}-2,2-dimethyl-tetrahydro-3aH-cyclopenta[d][1,3]dioxol-4-yl]methyl}carbamate 281c (0.51 g, 92%) was used in the next step directly without further purification. LCMS [conditions XBridge C18, 50*3.0 mm, 3.5 μm, Mobile Phase A: Water/0.05% Ammonia, Mobile Phase B: Acetonitrile, 5%-100% B-3 min (+).Icm, 1.20 mL/min, ES, m/z]: T_{R}=1.40 min; [M+H]⁺: 530.

### Ethyl-(E)-3-(4-amino-7-((3aS,4R,6R,6aR)-6-(((tert-butoxycarbonyl)amino)methyl)-2,2-dimethyltetrahydro-4H-cyclopenta[d][1,3]dioxol-4-yl)-7H-pyrrolo[2,3-d]pyrimidin-5-yl) acrylate (282c)

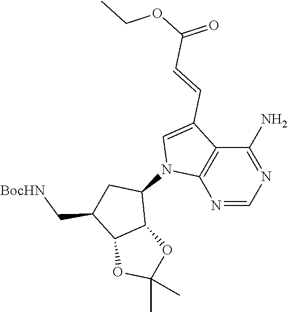 [SMILES: CCOC(=O)/C=C/c1cn([C@@H]2C[C@H](CNC(=O)OC(C)(C)C)[C@H]3OC(C)(C)O[C@H]32)c2ncnc(N)c12]

To a stirred mixture of tert-butyl-N-{[(3aR,4R,6R,6aS)-6-{4-amino-5-iodopyrrolo[2,3-d]pyrimidin-7-yl}-2,2-dimethyl-tetrahydro-3aH-cyclopenta[d][1,3]dioxol-4-yl]methyl}carbamate 281c (0.13 q, 0.25 mmol, 1.0 eq) and methyl-(2E)-3-(4,4,5,5-tetramethyl-1,3,2-dioxaborolan-2-yl)prop-2-enoate (78.0 mq, 0.37 mmol, 1.5 eq) in 1,4-dioxane (1.0 mL) and H₂O (0.1 mL) were added Pd (dppf) Cl₂·CH₂Cl₂ (20 mq, 0.03 mmol, 0.1 eq) and K₃PO₄ (0.16 q, 0.70 mmol, 3.0 eq) and the reaction mixture was stirred at 90° C. for 0.5 h, under N₂. The resulting mixture was concentrated under reduced pressure, partitioned between AcOEt and brine, and the organic layer was dried over anhydrous Na₂SO₄. The filtrate was concentrated under reduced pressure and the crude residue was purified by Prep-TLC (3:2 tetrahydrofuran: petroleum ether) to afford ethyl-(E)-3-(4-amino-7-((3aS,4R,6R,6aR)-6-(((tert-butoxycarbonyl)amino)methyl)-2,2-dimethyltetrahydro-4H-cyclopenta[d][1,3]dioxol-4-yl)-7H-pyrrolo[2,3-d]pyrimidin-5-yl) acrylate 282c (0.10 g, 84%) as a yellow solid. LCMS [conditions XBridge C18, 50*3.0 mm, 3.5 μm, Mobile Phase A: Water/0.05% Ammonia, Mobile Phase B: Acetonitrile, 5%-100% B-3 min (+).Icm, 1.20 mL/min, ES, m/z]: T_{R}=1.53 min; [M+H]⁺: 502.

### tert-Butyl-(((3aR,4R,6R,6aS)-2,2-dimethyl-6-(7-oxo-6,7-dihydro-2H-2,3,5,6-tetraazabenzo[cd]azulen-2-yl)tetrahydro-4H-cyclopenta[d][1,3]dioxol-4-yl)methyl)carbamate (283c)

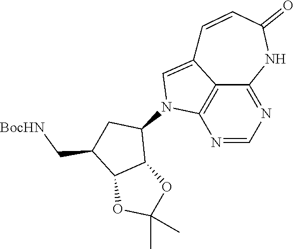 [SMILES: CC(C)(C)OC(=O)NC[C@H]1C[C@@H](n2cc3c4c(ncnc42)NC(=O)C=C3)[C@@H]2OC(C)(C)O[C@H]12]

To ethyl-(E)-3-(4-amino-7-((3aS,4R,6R,6aR)-6-(((tert-butoxycarbonyl)amino)methyl)-2,2-dimethyltetrahydro-4H-cyclopenta[d][1,3]dioxol-4-yl)-7H-pyrrolo[2,3-d]pyrimidin-5-yl) acrylate 282 (100 mq, 0.20 mmol, 1.0 eq) in MeOH (2.0 mL) was added NaOMe (33 mq, 0.60 mmol, 3.0 eq) at room temperature and the reaction mixture was stirred at 80° C. for 2 h. The resulting mixture was concentrated under reduced pressure and the crude residue was purified by silica gel column chromatography (1:1 tetrahydrofuran: petroleum ether) to afford tert-butyl-(((3aR,4R,6R,6aS)-2,2-dimethyl-6-(7-oxo-6,7-dihydro-2H-2,3,5,6-tetraazabenzo[cd]azulen-2-yl)tetrahydro-4H-cyclopenta[d][1,3]dioxol-4-yl)methyl)carbamate 283c (30 mg, 32%) as a yellow solid. LCMS [conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min (+).Icm, 1.50 mL/min, ES, m/z]: T_{R}=1.72 min; [M+H]⁺: 456.

### tert-Butyl-(((3aR,4R,6R,6aS)-2,2-dimethyl-6-(7-oxo-6,7,8,9-tetrahydro-2H-2,3,5,6-tetraazabenzo[cd]azulen-2-yl)tetrahydro-4H-cyclopenta[d][1,3]dioxol-4-yl)methyl)carbamate (284c)

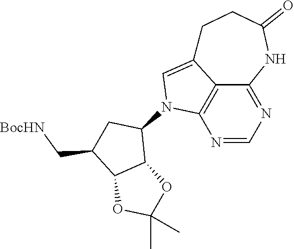 [SMILES: CC(C)(C)OC(=O)NC[C@H]1C[C@@H](n2cc3c4c(ncnc42)NC(=O)CC3)[C@@H]2OC(C)(C)O[C@H]12]

To a solution of tert-butyl-(((3aR,4R,6R,6aS)-2,2-dimethyl-6-(7-oxo-6,7-dihydro-2H-2,3,5,6-tetraazabenzo[cd]azulen-2-yl)tetrahydro-4H-cyclopenta[d][1,3]dioxol-4-yl)methyl)carbamate 283 (30.0 mg, 0.07 mmol, 1.0 eq) in MeOH (0.5 mL) was added 10% Pd/C (7.0 mg, 0.007 mmol, 0.1 eq) in a pressure tank and the reaction mixture was hydrogenated at room temperature under 30 psi of hydrogen pressure for 1 h. The resultant mixture was filtered through a Celite pad and the filtrate was concentrated under reduced pressure to afford tert-butyl-(((3aR,4R,6R,6aS)-2,2-dimethyl-6-(7-oxo-6,7,8,9-tetrahydro-2H-2,3,5,6-tetraazabenzo[cd]azulen-2-yl)tetrahydro-4H-cyclopenta[d][1,3]dioxol-4-yl)methyl)carbamate 284c (27.0 mg, 90%) which was used in the next step directly without further purification. LCMS [conditions Cortecs C18+, 50*3.0 mm, 2.7 Um, Mobile Phase A: Water/0.05% TFA, Mobile Phase B: Acetonitrile, 2%-100% B-2 min (+).Icm, 1.20 mL/min, ES, m/z]: T_{R}=0.99 min; [M+H]⁺: 458.

### 2-((1R,2S,3R,4R)-4-(Aminomethyl)-2,3-dihydroxycyclopentyl)-2,6,8,9-tetrahydro-7H-2,3,5,6-tetraazabenzo[cd]azulen-7-one (626)

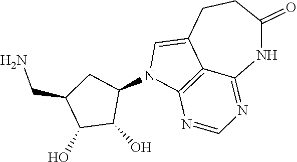 [SMILES: NC[C@H]1C[C@@H](n2cc3c4c(ncnc42)NC(=O)CC3)[C@H](O)[C@@H]1O]

To a stirred solution of tert-butyl-(((3aR,4R,6R,6aS)-2,2-dimethyl-6-(7-oxo-6,7,8,9-tetrahydro-2H-2,3,5,6-tetraazabenzo[cd]azulen-2-yl)tetrahydro-4H-cyclopenta[d][1,3]dioxol-4-yl)methyl)carbamate 284c (9.0 mg, 0.02 mmol, 1.0 eq) in MeOH (0.25 mL) was added 4M HCl in MeOH (0.5 mL) at room temperature and the reaction mixture was stirred for 1 h at room temperature. The resulting mixture was adjusted to pH 8 with HN3·H₂O and the crude residue was purified by Prep-HPLC [column, Xbridge Prep C18 OBD column, 5 μm, 19*150 mm; mobile phase, water (0.03% NH₄OH) and CH₃CN (20% CH₃CN up to 40% in 10 min); Detector, UV 220&254 nm]: to afford 2-((1R,2S,3R,4R)-4-(aminomethyl)-2,3-dihydroxycyclopentyl)-2,6,8,9-tetrahydro-7H-2,3,5,6-tetraazabenzo[cd]azulen-7-one 626 (1.9 mg, 30%) as a white solid. LCMS [conditions ZORBAX SB-Aq, 50*4.6 mm, 1.8 μm, Mobile Phase A: Water/0.02% TFA, Mobile Phase B: Acetonitrile, 5%-95% B-3 min-1.5.lcm, 1.50 mL/min, ES, m/z]: T_{R}=0.91 min; [M+H]⁺: 318. 1H NMR (300 MHZ, DMSO-d₆+D₂O) δ 8.42 (s, 1H), 7.40 (s, 1H), 5.00-4.85 (m, 1H), 4.21-4.10 (m, 1H), 3.78-3.76 (m, 1H), 3.53-3.49 (m, 1H), 2.97-2.94 (m, 2H), 2.91-2.83 (m, 2H), 2.31-2.25 (m, 1H), 2.22-1.91 (m, 2H), 1.54-1.48 (m, 1H).

### Synthesis of 2-((1R,2S,3R,4R)-2,3-dihydroxy-4-(((3-(phenethylamino)propyl)amino)methyl)cyclopentyl)-2,6,8,9-tetrahydro-7H-2,3,5,6-tetraazabenzo[cd]azulen-7-one (627)

### 2-((1R,2S,3R,4R)-2,3-dihydroxy-4-(((3-(phenethylamino)propyl)amino)methyl)cyclopentyl)-2,6,8,9-tetrahydro-7H-2,3,5,6-tetraazabenzo[cd]azulen-7-one (627)

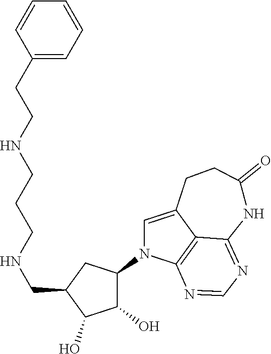 [SMILES: O=C1CCc2cn([C@@H]3C[C@H](CNCCCNCCc4ccccc4)[C@@H](O)[C@H]3O)c3ncnc(c23)N1]

To 2-((1R,2S,3R,4R)-4-(aminomethyl)-2,3-dihydroxycyclopentyl)-2,6,8,9-tetrahydro-7H-2,3,5,6-tetraazabenzo[cd]azulen-7-one 626 (16.0 mg, 0.05 mmol, 1.0 eq) in DCM (0.25 mL) were added NaBH (AcO) 3 (21.4 mg, 0.10 mmol, 2.0 eq) and AcOH (36.3 mg, 0.60 mmol, 12.0 eq) at room temperature and the resulting mixture was stirred for 30 min at ambient temperature. A solution of tert-butyl-N-(3-oxopropyl)-N-(2-phenylethyl)carbamate WO2021053158 (14.0 mg, 0.05 mmol, 1.0 eq) in DCM (0.25 mL) was added the reaction mixture was stirred for 0.5 h at room temperature. To the resulting mixture was added 4M HCl in MeOH (0.25 mL) and the reaction mixture was stirred for 30 min at room temperature. The resulting mixture was concentrated under reduced pressure, dissolved in DMF (2.0 mL) and adjusted to pH 9 with NH₃·H₂O. The crude residue was and was purified by Prep-HPLC [Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 15% B to 60% B in 7 min, 55% B; Wave Length: 220 nm]: to afford 2-((1R,2S,3R,4R)-2,3-dihydroxy-4-(((3-(phenethylamino)propyl)amino)methyl)cyclopentyl)-2,6,8,9-tetrahydro-7H-2,3,5,6-tetraazabenzo[cd]azulen-7-one 627 (3.4 mg, 14%) as an off-white solid. LCMS [conditions: ZORBAX SB-Aq, 50*4.6 mm, 1.8 μm, Mobile Phase A: Water/0.02% TFA, Mobile Phase B: Acetonitrile, 5%-95% B-3 min-1.5.lcm, 1.50 mL/min]: T_{R}=1.05 min; [M+H]⁺: 479. ¹H NMR (400 MHZ, DMSO-d₆+D₂O) δ 8.42 (s, 1H), 7.39 (s, 1H), 7.28-7.23 (m, 2H), 7.22-7.16 (m, 3H), 5.04-4.85 (m, 1H), 4.25-4.09 (m, 1H), 3.86-3.72 (m, 1H), 3.01-2.92 (m, 2H), 2.85-2.78 (m, 2H), 2.77-2.52 (m, 10H), 2.24-2.15 (m, 1H), 2.10-2.01 (m, 1H), 1.60-1.46 (m, 3H).

### Synthesis of (1S,2R,3R,5R)-3-(((3-(phenethylamino)propyl)amino)methyl)-5-(6,7,8,9-tetrahydro-2H-2,3,5,6-tetraazabenzo[cd]azulen-2-yl)cyclopentane-1,2-diol (628)

### tert-Butyl-N-[3-({[(3aR,4R,6R,6aS)-6-{4-amino-5-[(12)-3,3-diethoxyprop-1-en-1-yl]pyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate (285c)

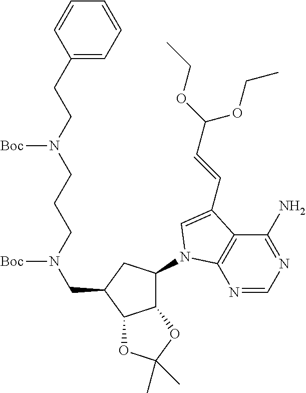 [SMILES: CCOC(/C=C/c1cn([C@@H]2C[C@H](CN(CCCN(CCc3ccccc3)C(=O)OC(C)(C)C)C(=O)OC(C)(C)C)[C@H]3OC(C)(C)O[C@H]32)c2ncnc(N)c12)OCC]

To a solution of tert-butyl-N-[3-({[(3aR,4R,6R,6aS)-6-{4-amino-5-bromopyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 108c (0.10 g, 0.13 mmol, 1.0 eq) and (E)-2-(3,3-diethoxyprop-1-en-1-yl)-4,4,5,5-tetramethyl-1,3,2-dioxaborolane (0.04 g, 0.16 mmol, 1.2 eq) in 1,4-dioxane (2.7 mL) and H₂O (0.3 mL) were added Pd (dtbpf) Cl₂ (8.76 mg, 0.013 mmol, 0.1 eq) and K₃PO₄ (0.08 g, 0.40 mmol, 3.0 eq), at room temperature under N₂, and the reaction mixture was stirred at 80° C. for 1 h. The reaction was quenched with water (5 mL) and then extracted with EtOAc (3×10 mL). The organic layer was dried over anhydrous Na₂SO₄, filtered and the filtrate was concentrated under reduced pressure to afford tert-butyl-N-[3-({[(3aR,4R,6R,6aS)-6-{4-amino-5-[(12)-3,3-diethoxyprop-1-en-1-yl]pyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 285c (100 mg, crude). LCMS [conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min (+), 1.50 L/min, ES, m/z]: T_{R}=2.43 min; [M+H]⁺: 793

### tert-Butyl-N-[3-({[(3aR,4R,6R,6aS)-6-[4-amino-5-(3,3-diethoxypropyl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate (286c)

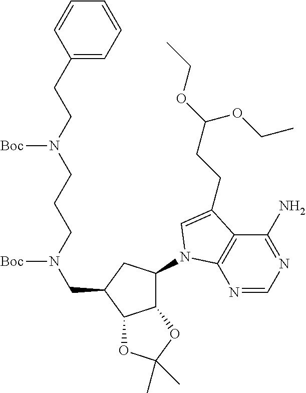 [SMILES: CCOC(CCc1cn([C@@H]2C[C@H](CN(CCCN(CCc3ccccc3)C(=O)OC(C)(C)C)C(=O)OC(C)(C)C)[C@H]3OC(C)(C)O[C@H]32)c2ncnc(N)c12)OCC]

To a solution of tert-butyl-N-[3-({[(3aR,4R,6R,6aS)-6-{4-amino-5-[(1E)-3,3-diethoxyprop-1-en-1-yl]pyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]--(2-phenylethyl)carbamate 285c (0.10 g, 0.10 mmol, 1.0 eq) in THF (0.5 mL) and EtOH (0.5 mL) was added 10% Pd/C (13.4 mg, 0.13 mmol, 1.0 eq) and the reaction mixture was stirred for 1 h at room temperature, under H₂. The resulting mixture was filtered and the filtrate was purified by Pre-HPLC [Column: Spherical C18, 20-40 μm, 120g; Mobile Phase A: Water, Mobile Phase B: ACN; Flow rate: 80 mL/min; Gradient: 30% B to 80% B in 10 min, 78% B; Wave Length: 254 nm]: to afford tert-butyl-N-[3-({[(3aR,4R,6R,6aS)-6-[4-amino-5-(3,3-diethoxypropyl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate 286c (40 mg, 40%). LCMS [conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min (+), 1.50 L/min, ES, m/z]: T_{R}=2.45 min; [M+H]⁺: 795

### tert-Butyl-(3-((tert-butoxycarbonyl)(((3aR,4R,6R,6aS)-2,2-dimethyl-6-(6,7,8,9-tetrahydro-2H-2,3,5,6-tetraazabenzo[cd]azulen-2-yl)tetrahydro-4H-cyclopenta[d][1,3]dioxol-4-yl)methyl)amino)propyl)(phenethyl)carbamate (287c)

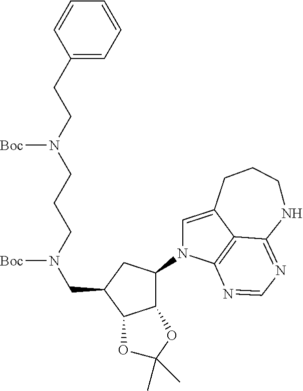 [SMILES: CC(C)(C)OC(=O)N(CCCN(C[C@H]1C[C@@H](n2cc3c4c(ncnc42)NCCC3)[C@@H]2OC(C)(C)O[C@H]12)C(=O)OC(C)(C)C)CCc1ccccc1]

To tert-butyl-N-[3-({[(3aR,4R,6R,6aS)-6-[4-amino-5-(3,3-diethoxypropyl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate 286c (30 mg, 0.04 mmol, 1.0 eq) was added AcOH (0.5 mL) at room temperature and the resulting mixture was stirred at 50° C. for 1 h. Then 10% Pd/C (4.02 mg, 0.04 mmol, 1.0 eq) was added and the reaction mixture was stirred for 8 h at room temperature, under H₂. The resulting mixture was filtered, the filter cake was washed with DMF (3×0.5 mL) and the combined filtrate, adjusted to pH 8 with NH₃·H₂O. The crude mixture was purified by Prep-HPLC [Column: YMC-Actus Triart C18, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 24% B to 49% B in 7 min, 49% B; Wave Length: 220 nm]: to afford tert-butyl-(3-((tert-butoxycarbonyl)(((3aR,4R,6R,6aS)-2,2-dimethyl-6-(6,7,8,9-tetrahydro-2H-2,3,5,6-tetraazabenzo[cd]azulen-2-yl)tetrahydro-4H-cyclopenta[d][1,3]dioxol-4-yl)methyl)amino)propyl)(phenethyl)carbamate 287c (18 mg, 27%) as a colorless oil. LCMS [conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min (+), 1.50 L/min, ES, m/z]: T_{R}=2.39 min; [M+H]⁺: 705

### (1S,2R,3R,5R)-3-(((3-(Phenethylamino)propyl)amino)methyl)-5-(6,7,8,9-tetrahydro-2H-2,3,5,6-tetraazabenzo[cd]azulen-2-yl)cyclopentane-1,2-diol (628)

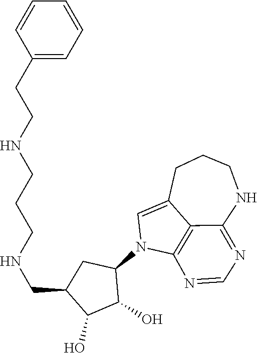 [SMILES: O[C@@H]1[C@@H](CNCCCNCCc2ccccc2)C[C@@H](n2cc3c4c(ncnc42)NCCC3)[C@@H]1O]

To tert-butyl-(3-((tert-butoxycarbonyl)(((3aR,4R,6R,6aS)-2,2-dimethyl-6-(6,7,8,9-tetrahydro-2H-2,3,5,6-tetraazabenzo[cd]azulen-2-yl)tetrahydro-4H-cyclopenta[d][1,3]dioxol-4-yl)methyl)amino)propyl)(phenethyl)carbamate 287c (18 mg, 0.03 mmol, 1.0 eq) was added 4M HCl in MeOH (2.0 mL, 8.00 mmol) and the reaction mixture was stirred for 3 h at room temperature. The resulting mixture was concentrated under reduced pressure, dissolved in MeOH (3.0 mL), adjusted to pH 8 with NH₃·H₂O and the crude residue was purified by Prep-HPLC [Column: YMC-Actus Triart C18, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 24% B to 49% B in 7 min, 49% B; Wave Length: 220 nm]: to afford (1S,2R,3R,5R)-3-(((3-(phenethylamino)propyl)amino)methyl)-5-(6,7,8,9-tetrahydro-2H-2,3,5,6-tetraazabenzo[cd]azulen-2-yl)cyclopentane-1,2-diol 628 (1.8 mg, 15%) as an off-white solid. LCMS [Xbridge BEH Phenyl, 50*3.0 mm, 2.5 μm, Mobile Phase A: Water/0.05% ammonia water, Mobile Phase B: CAN, 5%-100% B-3 min (+), 1.50 L/min, ES, m/z]: T_{R}=1.76 min; [M+H]⁺: 465. 1H NMR (400 MHZ, DMSO-d₆) δ 7.97 (s, 1H), 7.30-7.19 (m, 2H), 7.20-7.12 (m, 3H), 7.04 (s, 1H), 4.87-4.72 (m, 1H), 4.15-4.05 (m, 1H), 3.79-3.70 (m, 1H), 3.37-3.29 (m, 2H), 2.84-2.76 (m, 2H), 2.75-2.57 (m, 10H), 2.20-2.08 (m, 1H), 2.06-1.93 (m, 1H), 1.92-1.81 (m, 2H), 1.62-1.50 (m, 2H), 1.49-1.34 (m, 1H).

### Synthesis of (15,2R,3R,5R)-3-(Aminomethyl)-5-(9-methyl-6,7,8,9-tetrahydro-2H-2,3,5,6-tetraazabenzo[cd]azulen-2-yl)cyclopentane-1,2-diol (629)

### tert-Butyl-(((3aR,4R,6R,6aS)-6-(4-(but-3-en-1-ylamino)-5-iodo-7H-pyrrolo[2,3-d]pyrimidin-7-yl)-2,2-dimethyltetrahydro-4H-cyclopenta[d][1,3]dioxol-4-yl)methyl)carbamate (294c)

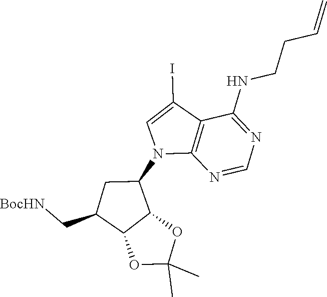 [SMILES: C=CCCNc1ncnc2c1c(I)cn2[C@@H]1C[C@H](CNC(=O)OC(C)(C)C)[C@H]2OC(C)(C)O[C@H]21]

To a solution of tert-butyl-N-{[(3aR,4R,6R,6aS)-6-{4-chloro-5-iodopyrrolo[2,3-d]pyrimidin-7-yl}-2,2-dimethyl-tetrahydro-3aH-cyclopenta[d][1,3]dioxol-4-yl]methyl}carbamate 280c (300 mg, 0.54 mmol, 1.0 eq) in DMA (5.0 mL) and TEA (165 mg, 1.64 mmol, 3.0 eq) was added 3-buten-1-amine (58 mg, 0.82 mmol, 1.5 eq) at room temperature and the reaction mixture was stirred at 100° C. for 16 h. The reaction was quenched with water (10 mL) and was then extracted with EtOAc (3×15 mL). The combined organic extracts were dried over anhydrous Na₂SO₄, filtered and the filtrate was concentrated under reduced pressure. The crude residue was purified by reverse flash chromatography [column, C18 silica gel; mobile phase, MeCN in Water, 10% to 50% gradient in 10 min; detector, UV 254 nm]: to afford (((3aR,4R,6R,6aS)-6-(4-(but-3-en-1-ylamino)-5-iodo-7H-pyrrolo[2,3-d]pyrimidin-7-yl)-2,2-dimethyltetrahydro-4H-cyclopenta[d][1,3]dioxol-4-yl)methyl)carbamate 294c (260 mg, 81%) as a yellow solid. LCMS [column: L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min, 1.20 mL/min, ES, m/z]: T_{R}=2.39 min; [M+H]⁺: 584.

### tert-Butyl-(((3aR,4R,6R,6aS)-2,2-dimethyl-6-(9-methylene-6,7,8,9-tetrahydro-2H-2,3,5,6-tetraazabenzo[cd]azulen-2-yl)tetrahydro-4H-cyclopenta[d][1,3]dioxol-4-yl)methyl)carbamate (295c)

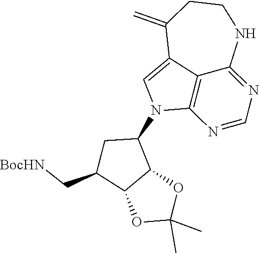 [SMILES: C=C1CCNc2ncnc3c2c1cn3[C@@H]1C[C@H](CNC(=O)OC(C)(C)C)[C@H]2OC(C)(C)O[C@H]21]

To a solution of (((3aR,4R,6R,6aS)-6-(4-(but-3-en-1-ylamino)-5-iodo-7H-pyrrolo[2,3-d]pyrimidin-7-yl)-2,2-dimethyltetrahydro-4H-cyclopenta[d][1,3]dioxol-4-yl)methyl)carbamate 294c (250 mg, 0.43 mmol, 1.0 eq) in DMA (3.0 mL) were added Pd (PPh₃) 2Cl₂ (60 mg, 0.08 mmol, 0.2 eq), Et₃N (130 mg, 1.30 mmol, 3.0 eq) and PPh₃ (337 mg, 1.28 mmol, 3.0 eq) and the reaction mixture was stirred at 120° C. for 2 h, under nitrogen. The resulting mixture was concentrated under reduced pressure and the crude residue was purified by silica gel column chromatography (1:1 Petroleum ether/AcOEt) to afford tert-butyl-(((3aR,4R,6R,6aS)-2,2-dimethyl-6-(9-methylene-6,7,8,9-tetrahydro-2H-2,3,5,6-tetraazabenzo[cd]azulen-2-yl)tetrahydro-4H-cyclopenta[d][1,3]dioxol-4-yl)methyl)carbamate 295c (56 mg, 28%) as a yellow crude solid. LCMS [column: L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min, 1.20 mL/min, ES, m/z]: T_{R}=2.23 min; [M+H]⁺: 456.

### tert-Butyl-(((3aR,4R,6R,6aS)-2,2-dimethyl-6-(9-methyl-6,7,8,9-tetrahydro-2H-2,3,5,6-tetraazabenzo[cd]azulen-2-yl)tetrahydro-4H-cyclopenta[d][1,3]dioxol-4-yl)methyl)carbamate (296c)

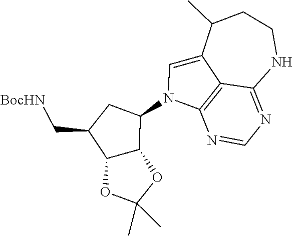 [SMILES: CC1CCNc2ncnc3c2c1cn3[C@@H]1C[C@H](CNC(=O)OC(C)(C)C)[C@H]2OC(C)(C)O[C@H]21]

To a solution of tert-butyl-(((3aR,4R,6R,6aS)-2,2-dimethyl-6-(9-methylene-6,7,8,9-tetrahydro-2H-2,3,5,6-tetraazabenzo[cd]azulen-2-yl)tetrahydro-4H-cyclopenta[d][1,3]dioxol-4-yl)methyl)carbamate 295c (20 mg, 0.04 mmol, 1.0 eq) in MeOH (2.0 mL) was added 10% Pd/C (5.0 mg) and the reaction mixture was stirred at room temperature for 1 h under a hydrogen balloon. The resulting mixture was filtered through a celite pad and concentrated under reduced pressure to afford tert-butyl-(((3aR,4R,6R,6aS)-2,2-dimethyl-6-(9-methyl-6,7,8,9-tetrahydro-2H-2,3,5,6-tetraazabenzo[cd]azulen-2-yl)tetrahydro-4H-cyclopenta[d][1,3]dioxol-4-yl)methyl)carbamate 296c (10 mg, crude) as a yellow solid. LCMS [column: XBridge C18, 50*3.0 mm, 3.5 μm, Mobile Phase A: Water/5 mM NH₄HCO₃, Mobile Phase B: Methanol, 10%-95% B-3 min, 1.00 mL/min, ES, m/z]: T_{R}=2.12 min; [M+H]⁺: 458.

### (1S,2R,3R,5R)-3-(Aminomethyl)-5-(9-methyl-6,7,8,9-tetrahydro-2H-2,3,5,6-tetraazabenzo[cd]azulen-2-yl)cyclopentane-1,2-diol (629)

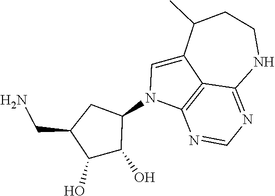 [SMILES: CC1CCNc2ncnc3c2c1cn3[C@@H]1C[C@H](CN)[C@@H](O)[C@H]1O]

To tert-butyl-(((3aR,4R,6R,6aS)-2,2-dimethyl-6-(9-methyl-6,7,8,9-tetrahydro-2H-2,3,5,6-tetraazabenzo[cd]azulen-2-yl)tetrahydro-4H-cyclopenta[d][1,3]dioxol-4-yl)methyl)carbamate 296c (10 mg, 0.02 mmol, 1.0 eq) was added TFA (2.0 mL) at room temperature and the reaction mixture was stirred at 50° C. for 1 h. The resulting mixture was concentrated under reduced pressure, adjusted to pH 8 with NH₃·H₂O and the crude mixture was purified by Prep-HPLC [Column, XBridge Shield RP18 OBD Column, 19*150 mm, 5 μm; mobile phase, Water (10 mmol/L NH₄HCO₃+0.1% NH₃·H₂O) and ACN (20% ACN up to 74% in 7 min); Detector, UV 254 nm]: to afford (1S,2R,3R,5R)-3-(aminomethyl)-5-(9-methyl-6,7,8,9-tetrahydro-2H-2,3,5,6-tetraazabenzo[cd]azulen-2-yl)cyclopentane-1,2-diol (629)(1.4 mg, 20%) as an off-white solid. LCMS [column: ZORBAX SB-Aq, 50*4.6 mm, 1.8 μm. mobile phase A: Water/0.02% TFA, mobile phase B: Acetonitrile, 5%-95% B-3 min, 1.50 mL/min, ES, m/z]: T_{R}=0.87 min; [M+H]⁺: 318. ¹H NMR (400 MHZ, DMSO-d₆+D₂O) δ 7.97 (s, 1H), 7.12 (s, 1H), 4.97-4.72 (m, 1H), 4.23-4.09 (m, 1H), 3.84-3.67 (m, 1H), 3.61-3.56 (m, 1H), 3.29-3.09 (m, 2H), 2.94-2.81 (m, 1H), 2.76-2.66 (m, 1H), 2.22-1.88 (m, 3H), 1.66-1.43 (m, 2H), 1.28 (d, J=6.6 Hz, 3H).

### Synthesis of (15,2R,3R,5R)-3-(aminomethyl)-5-(7,8,9,10-tetrahydro-2,3,5,6-tetraazacycloocta[cd]inden-2 (6H)-yl)cyclopentane-1,2-diol (630)

### tert-Butyl-(((3aR,4R,6R,6aS)-6-(4-amino-5-((E)-4-((tert-butyldimethylsilyl)oxy) but-1-en-1-yl)-7H-pyrrolo[2,3-d]pyrimidin-7-yl)-2,2-dimethyltetrahydro-4H-cyclopenta[d][1,3]dioxol-4-yl)methyl)carbamate (297c)

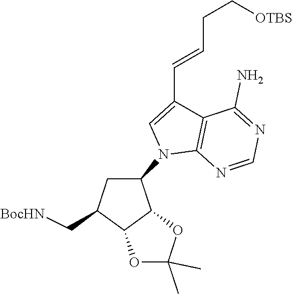 [SMILES: CC(C)(C)OC(=O)NC[C@H]1C[C@@H](n2cc(/C=C/CCO[Si](C)(C)C(C)(C)C)c3c(N)ncnc32)[C@@H]2OC(C)(C)O[C@H]12]

To a solution of tert-butyl-N-{[(3aR,4R,6R,6aS)-6-{4-amino-5-iodopyrrolo[2,3-d]pyrimidin-7-yl}-2,2-dimethyl-tetrahydro-3aH-cyclopenta[d][1,3]dioxol-4-yl]methyl}carbamate 281c (400 mg, 0.75 mmol, 1.0 eq) and tert-butyldimethyl {[(3E)-4-(4,4,5,5-tetramethyl-1,3,2-dioxaborolan-2-yl) but-3-en-1-yl]oxy}silane (354 mg, 1.10 mmol, 1.5 eq) in dioxane (5.0 mL) and H₂O (0.5 mL) were added Pd (dppf) Cl₂ (110 mg, 0.15 mmol, 0.2 eq) and K₃PO₄ (481 mg, 2.27 mmol, 3.0 eq) and the reaction mixture was stirred at 100° C. for 2 h under nitrogen. The reaction was quenched with water (10 mL) and then extracted with EtOAc (3×10 mL). The combined organic extracts were dried over anhydrous Na₂SO₄, filtered and the filtrate was concentrated under reduced pressure. The crude residue was purified by silica gel column chromatography (1:1 Petroleum ether/AcOEt) to afford tert-butyl-N-{[(3aR,4R,6R,6aS)-6-{4-amino-5-[(1E)-4-[(tert-butyldimethylsilyl)oxy]but-1-en-1-yl]pyrrolo[2,3-d]pyrimidin-7-yl}-2,2-dimethyl-tetrahydro-3aH-cyclopenta[d][1,3]dioxol-4-yl]methyl}carbamate 297c (290 mg, 65%) as a yellow solid. LCMS [column: XBridge C18, 50*3.0 mm, 3.5 μm, Mobile Phase A: Water/5 mM NH₄HCO₃, Mobile Phase B: Methanol, 10%-95% B-3 min, 1.00 mL/min, ES, m/z]: T_{R}=2.47 min; [M+H]⁺: 588.

### tert-Butyl-(((3aR,4R,6R,6aS)-6-(4-amino-5-(4-hydroxybutyl)-7H-pyrrolo[2,3-d]pyrimidin-7-yl)-2,2-dimethyltetrahydro-4H-cyclopenta[d][1,3]dioxol-4-yl)methyl)carbamate (298c) 1>

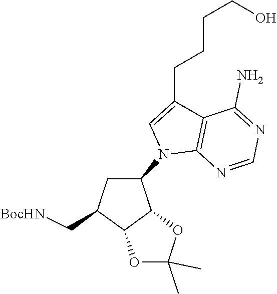 [SMILES: CC(C)(C)OC(=O)NC[C@H]1C[C@@H](n2cc(CCCCO)c3c(N)ncnc32)[C@@H]2OC(C)(C)O[C@H]12]

To a solution of tert-butyl-N-{[(3aR,4R,6R,6aS)-6-{4-amino-5-[(1E)-4-[(tert-butyldimethylsilyl)oxy]but-1-en-1-yl]pyrrolo[2,3-d]pyrimidin-7-yl}-2,2-dimethyl-tetrahydro-3aH-cyclopenta[d][1,3]dioxol-4-yl]methyl}carbamate 297c (200 mg, 0.34 mmol, 1.0 eq) in MeOH (4.0 mL) was added 10% Pd/C (18 mg) and the reaction mixture was stirred at room temperature for 1 h under a hydrogen balloon. The resulting mixture was filtered through a Celite pad and the filtrate was concentrated under reduced pressure. The resultant crude residue was dissolved in THF (4.0 mL), TBAF (1M in THF, 1.7 mL, 1.70 mmol, 5.0 eq) was added and the reaction mixture was stirred for 30 min at room temperature. The resulting mixture was concentrated under reduced pressure and the crude residue was purified by silica gel column chromatography (1:1 Petroleum ether/AcOEt) to afford tert-butyl-N-{[(3aR,4R,6R,6aS)-6-[4-amino-5-(4-hydroxybutyl)pyrrolo[2,3-d]pyrimidin-7-yl]-2,2-dimethyl-tetrahydro-3aH-cyclopenta[d][1,3]dioxol-4-yl]methyl}carbamate 298c (110 mg, 67%) as a yellow solid. LCMS [column: L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min, 1.20 mL/min, ES, m/z]: T_{R}=1.52 min; [M+H]⁺: 476.

### tert-Butyl-(((3aR,4R,6R,6aS)-6-(4-amino-5-(4-bromobutyl)-7H-pyrrolo[2,3-d]pyrimidin-7-yl)-2,2-dimethyltetrahydro-4H-cyclopenta[d][1,3]dioxol-4-yl)methyl)carbamate (299c)

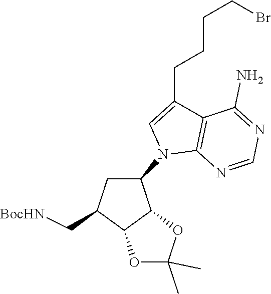 [SMILES: CC(C)(C)OC(=O)NC[C@H]1C[C@@H](n2cc(CCCCBr)c3c(N)ncnc32)[C@@H]2OC(C)(C)O[C@H]12]

To tert-butyl-N-{[(3aR,4R,6R,6aS)-6-[4-amino-5-(4-hydroxybutyl)pyrrolo[2,3-d]pyrimidin-7-yl]-2,2-dimethyl-tetrahydro-3aH-cyclopenta[d][1,3]dioxol-4-yl]methyl}carbamate 298c (110 mg, 0.23 mmol, 1.0 eq) in DCM (3.0 mL) were added PPh₃ (182 mg, 0.69 mmol, 3.0 eq), CBr₄ (153 mg, 0.46 mmol, 2.0 eq) and Na₂CO₃ (4.9 mg, 0.05 mmol, 0.2 eq), at room temperature under nitrogen, and the reaction mixture was stirred for 1 h at room temperature. The resulting mixture was concentrated under reduced pressure and the crude residue was purified by silica gel column chromatography (1:1 Petroleum ether/AcOEt) to afford tert-butyl-N-{[(3aR,4R,6R,6aS)-6-[4-amino-5-(4-bromobutyl)pyrrolo[2,3-d]pyrimidin-7-yl]-2,2-dimethyl-tetrahydro-3aH-cyclopenta[d][1,3]dioxol-4-yl]methyl}carbamate 299c (70 mg, 56%) as a yellow solid. LCMS [column: L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min, 1.20 mL/min, ES, m/z]: T_{R}=1.89 min; [M+H]⁺: 538, 540.

### tert-Butyl-(((3aR,4R,6R,6aS)-2,2-dimethyl-6-(7,8,9,10-tetrahydro-2,3,5,6-tetraazacycloocta [cd]inden-2 (6H)-yl)tetrahydro-4H-cyclopenta[d][1,3]dioxol-4-yl)methyl)carbamate (300c)

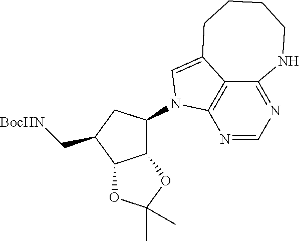 [SMILES: CC(C)(C)OC(=O)NC[C@H]1C[C@@H](n2cc3c4c(ncnc42)NCCCC3)[C@@H]2OC(C)(C)O[C@H]12]

To a solution of tert-butyl-N-{[(3aR,4R,6R,6aS)-6-[4-amino-5-(4-bromobutyl)pyrrolo[2,3-d]pyrimidin-7-yl]-2,2-dimethyl-tetrahydro-3aH-cyclopenta[d][1,3]dioxol-4-yl]methyl}carbamate 299c (70 mg, 0.13 mmol, 1.0 eq) in DMF (2.0 mL) was added NaH (12 mg, 0.52 mmol, 4.0 eq), in several portions over 10 min at 0° C., and the reaction mixture was stirred for 1 h at room temperature. The reaction was quenched with water at 0° C. and then extracted with EtOAc (3×10 mL). The combined organic extracts were dried over anhydrous Na₂SO₄, filtered and the filtrate was concentrated under reduced pressure. The crude residue was purified by silica gel column chromatography (1:1 Petroleum ether/AcOEt) to afford tert-butyl-(((3aR,4R,6R,6aS)-2,2-dimethyl-6-(7,8,9,10-tetrahydro-2,3,5,6-tetraazacycloocta [cd]inden-2 (6H)-yl)tetrahydro-4H-cyclopenta[d][1,3]dioxol-4-yl)methyl)carbamate 300c (40 mg, 67%) as a yellow solid. LCMS [column: L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min, 1.20 mL/min, ES, m/z]: T_{R}=1.82 min; [M+H]⁺: 458.

### (1S,2R,3R,5R)-3-(Aminomethyl)-5-(7,8,9,10-tetrahydro-2,3,5,6-tetraazacycloocta [cd]inden-2 (6H)-yl)cyclopentane-1,2-diol (630)

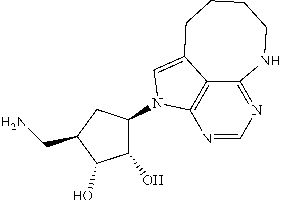 [SMILES: NC[C@H]1C[C@@H](n2cc3c4c(ncnc42)NCCCC3)[C@H](O)[C@@H]1O]

To tert-butyl-(((3aR,4R,6R,6aS)-2,2-dimethyl-6-(7,8,9,10-tetrahydro-2,3,5,6-tetraazacycloocta [cd]inden-2 (6H)-yl)tetrahydro-4H-cyclopenta[d][1,3]dioxol-4-yl)methyl)carbamate 300c (40 mg, 0.09 mmol, 1.0 eq) was added TFA (2.0 mL) at room temperature and the reaction mixture was stirred at 50° C. for 1 h. The resulting mixture was concentrated under reduced pressure, adjusted to pH 8 with NH₃ H₂O and the crude residue was purified by Prep-HPLC [Column, XBridge Shield RP18 OBD Column, 19*150 mm, 5 μm; mobile phase, Water (10 mmol/L NH₄HCO₃+0.1% NH₃·H₂O) and ACN (20% ACN up to 69% in 9.0 min); Detector, UV 254 nm]: to afford (1S,2R,3R,5R)-3-(aminomethyl)-5-(1S,2R,3R,5R)-3-(aminomethyl)-5-(7,8,9,10-tetrahydro-2,3,5,6-tetraazacycloocta [cd]inden-2 (6H)-yl)cyclopentane-1,2-diol (630)(15 mg, 54%) as an off-white solid. LCMS (column: ZORBAX SB-Aq, 50*4.6 mm, 1.8 μm. mobile phase A: Water/0.02% TFA, mobile phase B: Acetonitrile, 5%-95% B-3 min, 1.50 mL/min, ES, m/z): T_{R}=0.86 min; [M+H]⁺: 318. ¹H NMR (400 MHZ, DMSO-d₆+D₂O) δ 7.97 (s, 1H), 7.08 (s, 1H), 4.91-4.79 (m, 1H), 4.09 (q, J=7.1 Hz, 1H), 3.75 (q, J=4.9 Hz, 1H), 3.36-3.29 (m, 2H), 3.14-2.94 (m, 1H), 2.77-2.52 (m, 3H), 2.19-2.08 (m, 1H), 2.07-1.86 (m, 1H), 1.74-1.56 (m, 4H), 1.49-1.32 (m, 1H). Chiral-HPLC [Column Name: CHIRALPACK ID-3 50*3.0 mm,3.0 μm Co Solvent: B: MeOH (0.2% MSA), Start Conc. of Pump B: 30.0%; Oven Temperature: 35° C.; Total Flow: 1.00 mL/min]: T_{R}=0.95 min (3.0 min run), single peak.

### Synthesis of (15,2R,3R,5R)-3-(((3-(phenethylamino)propyl)amino)methyl)-5-(7,8,9,10-tetrahydro-2,3,5,6-tetraazacycloocta [cd]inden-2 (6H)-yl)cyclopentane-1,2-diol (631)

### (1S,2R,3R,5R)-3-(((3-(phenethylamino)propyl)amino)methyl)-5-(7,8,9,10-tetrahydro-2,3,5,6-tetraazacycloocta [cd]inden-2 (6H)-yl)cyclopentane-1,2-diol (631)

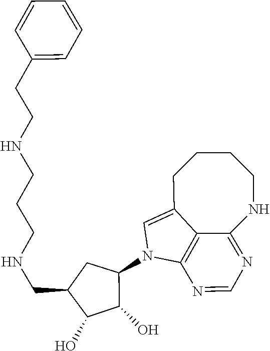 [SMILES: O[C@@H]1[C@@H](CNCCCNCCc2ccccc2)C[C@@H](n2cc3c4c(ncnc42)NCCCC3)[C@@H]1O]

To a solution of (1S,2R,3R,5R)-3-(aminomethyl)-5-(7,8,9,10-tetrahydro-2,3,5,6-tetraazacycloocta [cd]inden-2 (6H)-yl)cyclopentane-1,2-diol 630 (15 mg, 0.05 mmol, 1.0 eq), and tert-butyl-N-(3-oxopropyl)-N-(2-phenylethyl)carbamate 71c (16 mg, 0.06 mmol, 1.2 eq) in DCM (1.0 mL) was added AcOH (0.57 mg, 0.01 mmol, 0.2 eq) and the resulting mixture was stirred for 2 h at room temperature. To the above mixture was added NaBH₄ (3.0 mg, 0.07 mmol, 1.5 eq) and MeOH (1.0 mL) and the reaction mixture was stirred for 30 min at room temperature. To this mixture was then added TFA (2.0 mL) and the reaction mixture was stirred for 5 h at room temperature. The resulting mixture was concentrated under reduced pressure, adjusted to pH 8 with NH₃ H₂O and the crude residue was purified by Prep-HPLC [Column, XBridge Shield RP18 OBD Column, 19*150 mm, 5 μm; mobile phase, Water (10 mmol/L NH₄HCO₃+0.1% NH₃·H₂O) and ACN (20% ACN up to 72% in 9.0 min); Detector, UV 254 nm]: to afford (1S,2R,3R,5R)-3-(((3-(phenethylamino)propyl)amino)methyl)-5-(7,8,9,10-tetrahydro-2,3,5,6-tetraazacycloocta [cd]inden-2 (6H)-yl)cyclopentane-1,2-diol 631 (6.2 mg, 27%) as an off-white solid. LCMS [column: ZORBAX SB-Aq, 50*4.6 mm, 1.8 μm. mobile phase A: Water/0.02% TFA, mobile phase B: Acetonitrile, 5%-95% B-3 min, 1.50 mL/min, ES, m/z]: T_{R}=1.00 min; [M+H]⁺: 479. 1H NMR (400 MHZ, DMSO-d₆+D₂O) 7.96 (s, 1H), 7.29-7.21 (m, 2H), 7.20-7.11 (m, 3H), 7.08 (s, 1H), 4.84 (q, J=8.9 Hz, 1H), 4.10 (dd, J=8.2, 5.7 Hz, 1H), 3.73 (t, J=4.9 Hz, 1H), 3.34-3.30 (m, 2H), 2.75-2.52 (m, 12H), 2.20-2.08 (m, 1H), 2.03-1.99 (m, 1H), 1.71-1.66 (m, 2H), 1.65-1.58 (m, 2H), 1.57-1.49 (m, 2H), 1.48-1.35 (m, 1H). Chiral-HPLC [Column Name: CHIRALPAK IF-3 100*4.6 mm, 3 μm Co Solvent: B: ACN, Start Conc. of Pump B: 10.0%; Oven Temperature: 30° C.; Total Flow: 1.00 mL/min]: T_{R}=2.65 min (8.0 min run), single peak.

### Synthesis of (1R,2S,3R,5R)-3-(4-chloro-5-ethyl-7H-pyrrolo[2,3-d]pyrimidin-7-yl)-5-(((3-(phenethylamino)propyl)amino)methyl)cyclopentane-1,2-diol (632)

### tert-Butyl-(3-((tert-butoxycarbonyl)(phenethyl)amino)propyl)(((3aR,4R,6R,6aS)-6-(4-chloro-5-vinyl-7H-pyrrolo[2,3-d]pyrimidin-7-yl)-2,2-dimethyltetrahydro-4H-cyclopenta[d][1,3]dioxol-4-yl)methyl)carbamate (301c)

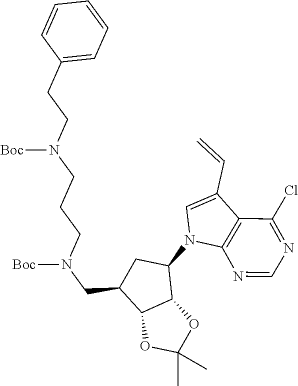 [SMILES: C=Cc1cn([C@@H]2C[C@H](CN(CCCN(CCc3ccccc3)C(=O)OC(C)(C)C)C(=O)OC(C)(C)C)[C@H]3OC(C)(C)O[C@H]32)c2ncnc(Cl)c12]

To a solution of tert-butyl-N-[3-({[(3aR,4R,6R,6aS)-6-{5-bromo-4-chloropyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 129c (70 mg, 0.09 mmol, 1.0 eq) and 2-ethenyl-4,4,5,5-tetramethyl-1,3,2-dioxaborolane (14.1 mg, 0.09 mmol, 1.0 eq) in dioxane (2.5 mL) and H₂O (0.25 mL) were added Pd (DtBPF) Cl₂ (6.0 mg, 0.009 mmol, 0.1 eq) and K₃PO₄ (58.4 mg, 0.30 mmol, 3.0 eq) and the reaction mixture was stirred at 60° C. for 0.5 h, under N₂. The reaction was quenched with water (5 mL) and then extracted with EtOAc (3×10 mL). The combined organic extracts were dried over anhydrous Na₂SO₄, filtered and the filtrate was concentrated under reduced pressure. The crude residue was purified by silica gel column chromatography (3:7 tetrahydrofuran: Petroleum ether) to afford tert-butyl-N-[3-({[(3aR,4R,6R,6aS)-6-{4-chloro-5-ethenylpyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 301c (50 mg, 77%) as a pale yellow oil. LCMS [conditions: L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min (+).lcm]: T_{R}=2.65 min; [M+H]⁺: 710, 712.

### tert-Butyl-(3-((tert-butoxycarbonyl)(phenethyl)amino)propyl)(((3aR,4R,6R,6aS)-6-(4-chloro-5-ethyl-7H-pyrrolo[2,3-d]pyrimidin-7-yl)-2,2-dimethyltetrahydro-4H-cyclopenta[d][1,3]dioxol-4-yl)methyl)carbamate (302c)

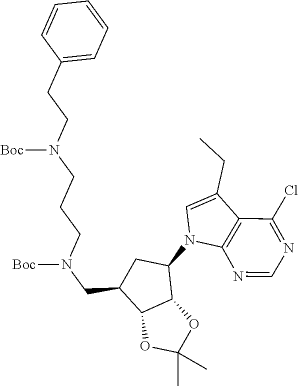 [SMILES: CCc1cn([C@@H]2C[C@H](CN(CCCN(CCc3ccccc3)C(=O)OC(C)(C)C)C(=O)OC(C)(C)C)[C@H]3OC(C)(C)O[C@H]32)c2ncnc(Cl)c12]

To a solution of tert-butyl-N-[3-({[(3aR,4R,6R,6aS)-6-{4-chloro-5-ethenylpyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 301c (50 mg, 0.07 mmol, 1.0 eq) in THF (3.0 mL) was added 10% Pd/C (3.7 mg, 0.007 mmol, 0.1 eq) in a pressure tank and the reaction mixture was hydrogenated at room temperature, under 30 psi of hydrogen pressure, for min. The resulting mixture was filtered through a celite pad and the filtrate was concentrated under reduced pressure. The crude residue was purified by silica gel column chromatography (2:3 ethyl acetate: Petroleum ether) to afford tert-butyl-N-[3-({[(3aR,4R,6R,6aS)-6-{4-chloro-5-ethylpyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 302c (35 mg, 70%) as a colorless oil. LCMS [conditions: Proshell HPH-C18, 50*3.0 mm, 2.7 μm, Mobile Phase A: Water (0.05% ammonia water), Mobile Phase B: Acetonitrile, 5%-100% B-2 min (+).lcm]: T_{R}=1.69 min; [M+H]⁺: 712, 714.

### (1R,2S,3R,5R)-3-(4-chloro-5-ethyl-7H-pyrrolo[2,3-d]pyrimidin-7-yl)-5-(((3-(phenethylamino)propyl)amino)methyl)cyclopentane-1,2-diol (632)

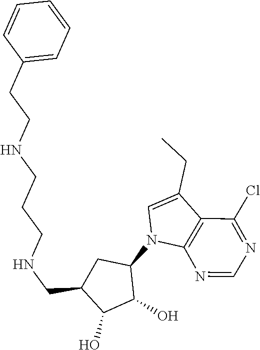 [SMILES: CCc1cn([C@@H]2C[C@H](CNCCCNCCc3ccccc3)[C@@H](O)[C@H]2O)c2ncnc(Cl)c12]

To tert-butyl-N-[3-({[(3aR,4R,6R,6aS)-6-{4-chloro-5-ethylpyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 302c (33 mg, 0.05 mmol, 1.0 eq) was added TFA (1.0 mL) and the reaction mixture was stirred at 50° C. for 1 h. The resulting mixture was concentrated under reduced pressure and adjusted to pH 8 with NH₃·H₂O. The crude residue was dissolved in DMF (3.0 mL) and was then purified by Prep-HPLC [Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 30 ml/min; Gradient: 25% B to 65% B in 9 min, 55% B; Wave Length: 220 nm]: to afford (1R,2S,3R,5R)-3-(4-chloro-5-ethyl-7H-pyrrolo[2,3-d]pyrimidin-7-yl)-5-(((3-(phenethylamino)propyl)amino)methyl)cyclopentane-1,2-diol 632 (14 mg, 64%) as a white solid. LCMS [conditions ZORBAX SB-Aq, 50*4.6 mm, 1.8 μm, Mobile Phase A: Water/0.02% TFA, Mobile Phase B: MeOH, 5%-95% B-5 min-1.0-AB.lcm, 1.50 mL/min, ES, m/z]: T_{R}=2.76 min; [M+H]⁺: 472, 474. ¹H NMR (400 MHZ, DMSO-d₆+D₂O) δ 8.53 (s, 1H), 7.65 (s, 1H), 7.29-7.21 (m, 2H), 7.20-7.12 (m, 3H), 4.98 (dd, J=10.7, 8.3 Hz, 1H), 4.21 (dd, J=8.7, 5.6 Hz, 1H), 3.77 (dd, J=5.6, 3.5 Hz, 1H), 2.87 (q, J=7.4 Hz, 2H), 2.81-2.52 (m, 10H), 2.25-2.13 (m, 1H), 2.11-2.00 (m, 1H), 1.61-1.48 (m, 3H), 1.28 (t, J=7.4 Hz, 3H).

### Synthesis of (1R,2S,3R,5R)-3-(5-(4-chloro-1H-pyrazol-5-yl)-7H-pyrrolo[2,3-d]pyrimidin-7-yl)-5-(((3-(phenethylamino)propyl)amino)methyl)cyclopentane-1,2-diol (633)

### tert-Butyl-(3-((tert-butoxycarbonyl)(phenethyl)amino)propyl)(((3aR,4R,6R,6aS)-6-(5-iodo-7H-pyrrolo[2,3-d]pyrimidin-7-yl)-2,2-dimethyltetrahydro-4H-cyclopenta[d][1,3]dioxol-4-yl)methyl)carbamate (303c)

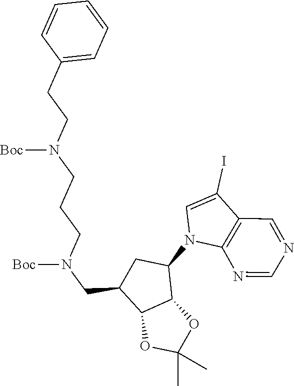 [SMILES: CC(C)(C)OC(=O)N(CCCN(C[C@H]1C[C@@H](n2cc(I)c3cncnc32)[C@@H]2OC(C)(C)O[C@H]12)C(=O)OC(C)(C)C)CCc1ccccc1]

To a solution of tert-butyl-(3-((tert-butoxycarbonyl)(phenethyl)amino)propyl)(((3aR,4R,6R,6aS)-2,2-dimethyl-6-(7H-pyrrolo[2,3-d]pyrimidin-7-yl)tetrahydro-4H-cyclopenta[d][1,3]dioxol-4-yl)methyl)carbamate 219c (0.15 g, 0.25 mmol, 1.0 eq) in DMF (2.0 mL) was added NIS (62.3 mg, 0.30 mmol, 1.2 eq) at room temperature and the reaction mixture was stirred at room temperature for 2 h. The reaction was quenched by the addition of saturated sodium thiosulfate solution and the resulting mixture was extracted with ethyl acetate (3×10 mL). The combined organic extracts were washed with brine, dried over anhydrous Na₂SO₄ and the filtrate was concentrated under reduced pressure. The crude residue was purified by silica gel column chromatography (2:3 Ethyl acetate: Petroleum ether) to afford tert-butyl-(3-((tert-butoxycarbonyl)(phenethyl)amino)propyl) (((3aR,4R,6R,6aS)-6-(5-iodo-7H-pyrrolo[2,3-d]pyrimidin-7-yl)-2,2-dimethyltetrahydro-4H-cyclopenta[d][1,3]dioxol-4-yl)methyl)carbamate 303c (0.11 g, 60%) as a colorless oil. LCMS (conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-2 min (+).lcm, 1.50 mL/min, ES, m/z): T_{R}=1.86 min; [M+H]⁺: 776.

### (4-Chloro-1-((2-(trimethylsilyl) ethoxy)methyl)-1H-pyrazol-5-yl) zinc (II) chloride (304c)

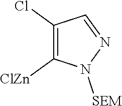

To a stirred solution of 4-chloro-1-{[2-(trimethylsilyl)ethoxy]methyl}pyrazole (0.40 g, 1.70 mmol, 1.0 eq) [J. Org. Chem., 2016, 81 (2), 689-698]in THF (4.0 mL) was added 2,2,6,6-tetramethylpiperidinylmagnesium chloride-lithium chloride complex (1.9 mL, 1 M in THF, 1.90 mmol, 1.1 eq), dropwise, at −20° C., under N₂ and the reaction mixture was stirred at −20° C. for 0.5 h. To the above mixture was added ZnCl₂ (3.0 mL, 0.7 M in THF, 2.10 mmol, 1.2 eq) dropwise, at −20° C. and the reaction mixture was stirred at 10° C. for 1 h. The resulting mixture was used in the next step directly without isolation.

### tert-Butyl-(3-((tert-butoxycarbonyl)(phenethyl)amino)propyl)(((3aR,4R,6R,6aS)-6-(5-(4-chloro-1-((2-(trimethylsilyl) ethoxy)methyl)-1H-pyrazol-5-yl)-7H-pyrrolo[2,3-d]pyrimidin-7-yl)-2,2-dimethyltetrahydro-4H-cyclopenta[d][1,3]dioxol-4-yl)methyl)carbamate (305c)

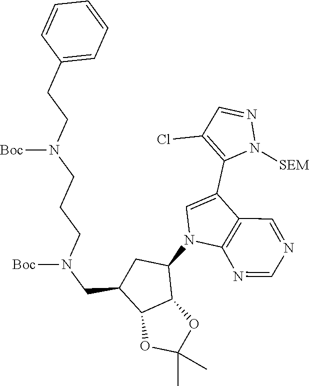 [SMILES: CC(C)(C)OC(=O)N(CCCN(C[C@H]1C[C@@H](n2cc(-c3c(Cl)cnn3COCC[Si](C)(C)C)c3cncnc32)[C@@H]2OC(C)(C)O[C@H]12)C(=O)OC(C)(C)C)CCc1ccccc1]

To a stirred solution of tert-butyl-(3-((tert-butoxycarbonyl)(phenethyl)amino)propyl) (((3aR,4R,6R,6aS)-6-(5-iodo-7H-pyrrolo[2,3-d]pyrimidin-7-yl)-2,2-dimethyltetrahydro-4H-cyclopenta[d][1,3]dioxol-4-yl)methyl)carbamate 303c (0.10 g, 0.13 mmol, 1.0 eq), Pd₂ (dba)₃ (11.8 mg, 0.013 mmol, 0.1 eq), tris(furan-2-yl)phosphane (6.0 mg, 0.03 mmol, 0.2 eq) and K₃PO₄ (82.1 mg, 0.40 mmol, 3.0 eq) in THF (5.0 mL) was added chloro(4-chloro-2-{[2-(trimethylsilyl) ethoxy]methyl}pyrazol-3-yl) zinc 304c (1.7 mL, crude, 0.33 mmol, 2.5 eq), dropwise, at room temperature, under N₂ and the reaction mixture was stirred at 60° C. for 12 h. The reaction was quenched with saturated ammonium chloride solution at room temperature and then extracted with ethyl acetate (3×20 mL). The combined organic extracts were washed with saturated brine and dried over anhydrous Na₂SO₄. The filtrate was concentrated under reduced pressure and the crude residue was purified by silica gel column chromatography (1:1 Ethyl acetate: Petroleum ether) to afford tert-butyl-(3-((tert-butoxycarbonyl)(phenethyl)amino)propyl) (((3aR,4R,6R,6aS)-6-(5-(4-chloro-1-((2-(trimethylsilyl) ethoxy)methyl)-1H-pyrazol-5-yl)-7H-pyrrolo[2,3-d]pyrimidin-7-yl)-2,2-dimethyltetrahydro-4H-cyclopenta[d][1,3]dioxol-4-yl)methyl)carbamate 305c (76 mg, 67%) as a white solid. LCMS [conditions Cortecs C18+, 50*3.0 mm, 2.7 μm, Mobile Phase A: Water/0.05% TFA, Mobile Phase B: Acetonitrile, 2%-95% B-3 min (+)(90-2000).Icm, 1.50 mL/min, ES, m/z]: T_{R}=2.28 min; [M+H]⁺: 880, 882.

### (1R,2S,3R,5R)-3-(5-(4-Chloro-1H-pyrazol-5-yl)-7H-pyrrolo[2,3-d]pyrimidin-7-yl)-5-(((3-(phenethylamino)propyl)amino)methyl)cyclopentane-1,2-diol (633)

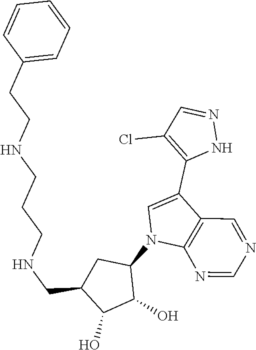 [SMILES: O[C@@H]1[C@@H](CNCCCNCCc2ccccc2)C[C@@H](n2cc(-c3[nH]ncc3Cl)c3cncnc32)[C@@H]1O]

To tert-butyl-(3-((tert-butoxycarbonyl)(phenethyl)amino)propyl)(((3aR,4R,6R,6aS)-6-(5-(4-chloro-1-((2-(trimethylsilyl) ethoxy)methyl)-1H-pyrazol-5-yl)-7H-pyrrolo[2,3-d]pyrimidin-7-yl)-2,2-dimethyltetrahydro-4H-cyclopenta[d][1,3]dioxol-4-yl)methyl)carbamate 305c (70 mg, 0.08 mmol, 1.0 eq) was added TFA (1.0 mL) and the reaction mixture was stirred at 50° C. for 1 h. The resulting mixture was concentrated under reduced pressure, dissolved in DMF (3.0 mL), adjusted the solution to pH 8 with NH₃·H₂O and the crude residue was purified by Prep-HPLC [Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 15% B to 55% B in 7 min, 55% B; Wave Length: 220 nm]: to afford (1R,2S,3R,5R)-3-(5-(4-chloro-1H-pyrazol-5-yl)-7H-pyrrolo[2,3-d]pyrimidin-7-yl)-5-(((3-(phenethylamino)propyl)amino)methyl)cyclopentane-1,2-diol 633 (25.3 mg, 62%) as a white solid. LCMS [conditions ZORBAX SB-Aq, 50*4.6 mm, 1.8 μm, Mobile Phase A: Water/0.02% TFA, Mobile Phase B: Acetonitrile, 5%-95% B-3 min-1.5.lcm, 1.50 mL/min, ES, m/z]: T_{R}=1.07 min; [M+H]⁺: 510, 512. ¹H NMR (400 MHZ, DMSO-d₆+D₂O) ¹H NMR (400 MHZ, DMSO-d₆) δ 9.36 (s, 1H), 8.85 (s, 1H), 8.17 (s, 1H), 8.03 (s, 1H), 7.27-7.11 (m, 5H), 5.10-4.99 (m, 1H), 4.33 (dd, J=8.7, 5.5 Hz, 1H), 3.81 (dd, J=5.6, 3.5 Hz, 1H), 2.77-2.53 (m, 10H), 2.34-2.24 (m, 1H), 2.15-2.02 (m, 1H), 1.76-1.64 (m, 1H), 1.59-1.51 (m, 2H). Chiral-SFC [Column Name: CHIRALPACK IH-3 50*3.0 mm,3.0 μm, Mobile Phase A: Water (0.05% H₃PO₄), Solvent: B: acetontirle, Start Conc. of Pump B: 10.0%; Oven Temperature: 25° C.; Total Flow: 1.00 mL/min]: T_{R}=1.20 min (3 min run), single peak.

### Synthesis of (1R,2S,3R,5R)-3-(5-(4-benzylthiazol-2-yl)-2-chloro-7H-pyrrolo[2,3-d]pyrimidin-7-yl)-5-(((3-(phenethylamino)propyl)amino)methyl)cyclopentane-1,2-diol (634)

### 4-Benzyl-2-(2-chloro-7H-pyrrolo[2,3-d]pyrimidin-5-yl) thiazole (306c)

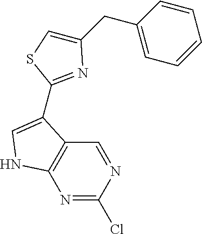 [SMILES: Clc1ncc2c(-c3nc(Cc4ccccc4)cs3)c[nH]c2n1]

To a solution of 2-chloro-5-iodo-7H-pyrrolo[2,3-d]pyrimidine (0.10 g, 0.36 mmol, 1.0 eq) and (4-benzyl-1,3-thiazol-2-yl)(chloro) zinc 227c (2.2 mL, crude, 0.72 mmol, 2.0 eq) in toluene (2.0 mL) was added Pd (PPh₃) 2Cl₂ (25.1 mg, 0.04 mmol, 0.1 eq) and the the reaction mixture was stirred at 60° C. for 12 h, under N₂. The reaction was quenched with ammonium chloride solution at 0° C. and the resulting mixture was extracted with ethyl acetate (3×10 mL). The combined organic extracts were washed with brine, dried over anhydrous Na₂SO₄ and the filtrate was concentrated under reduced pressure. The crude residue was purified by reverse flash chromatography (C18 silica gel; mobile phase, Water/0.05% TFA in acetonitrile, 10% to 50% gradient in 12 min; detector, UV 254 nm): to afford 4-benzyl-2-{2-chloro-7H-pyrrolo[2,3-d]pyrimidin-5-yl}-1,3-thiazole 306c (50 mg, 43%) as an off white solid. LCMS [conditions: L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min (+).lcm,]: T_{R}=1.36 min; [M+H]⁺: 327, 329.

### tert-Butyl-(3-((((3aR,4R,6R,6aS)-6-(5-(4-benzylthiazol-2-yl)-2-chloro-7H-pyrrolo[2,3-d]pyrimidin-7-yl)-2,2-dimethyltetrahydro-4H-cyclopenta[d][1,3]dioxol-4-yl)methyl)(tert-butoxycarbonyl)amino)propyl)(phenethyl)carbamate (307c)

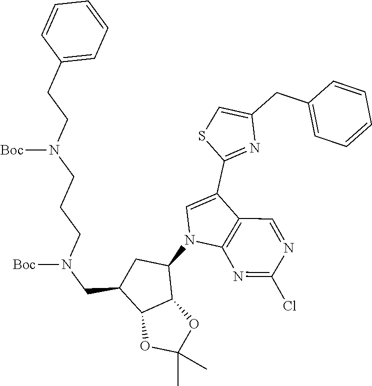 [SMILES: CC(C)(C)OC(=O)N(CCCN(C[C@H]1C[C@@H](n2cc(-c3nc(Cc4ccccc4)cs3)c3cnc(Cl)nc32)[C@@H]2OC(C)(C)O[C@H]12)C(=O)OC(C)(C)C)CCc1ccccc1]

To a stirred solution of 4-benzyl-2-{2-chloro-7H-pyrrolo[2,3-d]pyrimidin-5-yl}-1,3-thiazole (50 mg, 0.15 mmol, 1.0 eq), tert-butyl-N-[3-({[(3aR,4R,6S,6aS)-6-hydroxy-2,2-dimethyl-tetrahydro-3aH-cyclopenta[d][1,3]dioxol-4-yl]methyl}(tert-butoxycarbonyl)amino)propyl]-N-(2-phenylethyl)carbamate 306c (0.10 g, 0.20 mmol, 1.2 eq) and PPh₃ (80.3 mg, 0.30 mmol, 2.0 eq) in toluene (1.0 mL) was added a solution of DBAD (70.5 mg, 0.30 mmol, 2.0 eq) in toluene (0.5 mL), dropwise, at 0° C. and the reaction mixture was stirred at 60° C. for 4 h. The resulting mixture was concentrated under vacuum and the crude residue was purified by silica gel column chromatography (1:1 tetrahydrofuran: petroleum ether) to afford tert-butyl-(3-((((3aR,4R,6R,6aS)-6-(5-(4-benzylthiazol-2-yl)-2-chloro-7H-pyrrolo[2,3-d]pyrimidin-7-yl)-2,2-dimethyltetrahydro-4H-cyclopenta[d][1,3]dioxol-4-yl)methyl)(tert-butoxycarbonyl)amino)propyl)(phenethyl)carbamate 307c (80 mg, 61%) as a colorless oil. LCMS [conditions Cortecs C18+, 50*3.0 mm, 2.7 μm, Mobile Phase A: Water/0.05% TFA, Mobile Phase B: Acetonitrile, 2%-95% B-3 min (+), 1.50 mL/min, ES, m/z]: T_{R}=2.13 min; [M+H]⁺: 857, 859.

### (1R,2S,3R,5R)-3-(5-(4-Benzylthiazol-2-yl)-2-chloro-7H-pyrrolo[2,3-d]pyrimidin-7-yl)-5-(((3-(phenethylamino)propyl)amino)methyl)cyclopentane-1,2-diol (634)

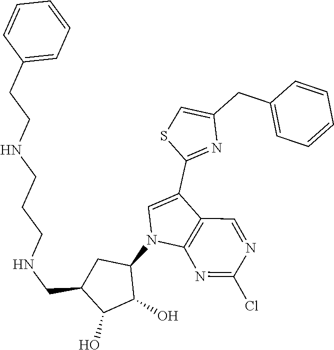 [SMILES: O[C@@H]1[C@@H](CNCCCNCCc2ccccc2)C[C@@H](n2cc(-c3nc(Cc4ccccc4)cs3)c3cnc(Cl)nc32)[C@@H]1O]

To tert-butyl-N-[3-({[(3aR,4R,6R,6aS)-6-[5-(4-benzyl-1,3-thiazol-2-yl)-2-chloropyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate 307c (60.0 mg, 0.07 mmol, 1.0 eq) was added 4M HCl in MeOH (2.0 mL, 8.00 mmol) at room temperature and the reaction mixture was stirred at 50° C. for 0.5 h. The resulting mixture was concentrated under reduced pressure, dissolved in MeOH (3.0 mL) and the solution was adjusted to pH 8 with Na₂CO₃. The crude residue was purified by Prep-HPLC [Column: YMC Triart C18, 30*150 mm, 5 μm; Mobile Phase A: 20 mM NH₄HCO₃+0.05% NH₃H₂O, Mobile Phase B: ACN; Flow rate: 35 mL/min; Gradient: 15% to 70% in 8 min]: to afford (1R,2S,3R,5R)-3-(5-(4-benzylthiazol-2-yl)-2-chloro-7H-pyrrolo[2,3-d]pyrimidin-7-yl)-5-(((3-(phenethylamino)propyl)amino)methyl)cyclopentane-1,2-diol 634 (15.0 mg, 35%) as a white solid. LCMS [conditions ZORBAX SB-Aq, 50*4.6 mm, 1.8 μm, Mobile Phase A: Water/0.02% TFA, Mobile Phase B: Acetonitrile, 5%-95% B-3 min-1.5.lcm, 1.50 mL/min]: T_{R}=1.42 min; [M+H]⁺: 617, 619. ¹H NMR (400 MHZ, DMSO-d₆+D₂O) δ 9.31 (s, 1H), 8.59 (s, 1H), 7.40-7.30 (m, 4H), 7.28-7.13 (m, 7H), 5.06-4.89 (m, 2H), 4.35-4.25 (m, 1H), 4.15 (s, 2H), 3.85-3.75 (m, 1H), 2.78-2.56 (m, 10H), 2.28-2.19 (m, 1H), 2.13-2.03 (m, 1H), 1.65-1.53 (m, 3H).

### Synthesis of (1R,2S,3R,5R)-3-(4-Amino-5-(4-benzylthiazol-2-yl)-2-chloro-7H-pyrrolo[2,3-d]pyrimidin-7-yl)-5-(((3-(phenethylamino)propyl)amino)methyl)cyclopentane-1,2-diol (635)

### tert-Butyl-(3-((tert-butoxycarbonyl)(((3aR,4R,6R,6aS)-6-(2,4-dichloro-5-iodo-7H-pyrrolo[2,3-d]pyrimidin-7-yl)-2,2-dimethyltetrahydro-4H-cyclopenta[d][1,3]dioxol-4-yl)methyl)amino)propyl)(phenethyl)carbamate (308r)

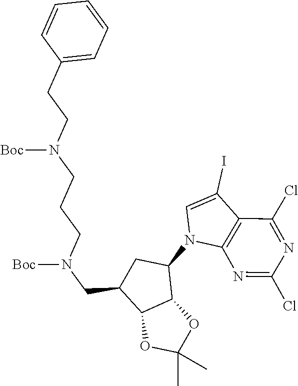 [SMILES: CC(C)(C)OC(=O)N(CCCN(C[C@H]1C[C@@H](n2cc(I)c3c(Cl)nc(Cl)nc32)[C@@H]2OC(C)(C)O[C@H]12)C(=O)OC(C)(C)C)CCc1ccccc1]

To a stirred solution of tert-butyl-N-[3-({[(3aR,4R,6S,6aS)-6-hydroxy-2,2-dimethyl-tetrahydro-3aH-cyclopenta[d][1,3]dioxol-4-yl]methyl}(tert-butoxycarbonyl)amino)propyl]-N-(2-phenylethyl)carbamate 256c (0.21 g, 0.38 mmol, 1.2 eq), 2,4-dichloro-5-iodo-7H-pyrrolo[2,3-d]pyrimidine (0.10 g, 0.30 mmol, 1.0 eq) and PPh₃ (0.17 g, 0.60 mmol, 2.0 eq) in toluene (1.0 mL) was added a solution of DBAD (0.15 g, 0.60 mmol, 2.0 eq) in toluene (1.0 mL), dropwise, at 0° C. under N₂, and the reaction mixture was stirred at 60° C. for 2 h. The resulting mixture was allowed to cool down to room temperature and was concentrated under reduced pressure. The crude residue was dissolved in DCM (2.0 mL) and was purified by silica gel column chromatography (1:2 ethyl acetate:petroleum ether) to afford tert-butyl-(3-((tert-butoxycarbonyl)(((3aR,4R,6R,6aS)-6-(2,4-dichloro-5-iodo-7H-pyrrolo[2,3-d]pyrimidin-7-yl)-2,2-dimethyltetrahydro-4H-cyclopenta[d][1,3]dioxol-4-yl)methyl)amino)propyl)(phenethyl)carbamate 308c (0.10 g, 37%) as a yellow solid. LCMS [conditions: L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min (+).lcm]: T_{R}=2.15 min; [M+H]⁺: 844, 846.

### tert-Butyl-(3-((((3aR,4R,6R,6aS)-6-(4-amino-2-chloro-5-iodo-7H-pyrrolo[2,3-d]pyrimidin-7-yl)-2,2-dimethyltetrahydro-4H-cyclopenta[d][1,3]dioxol-4-yl)methyl)(tert-butoxycarbonyl)amino)propyl)(phenethyl)carbamate (309c)

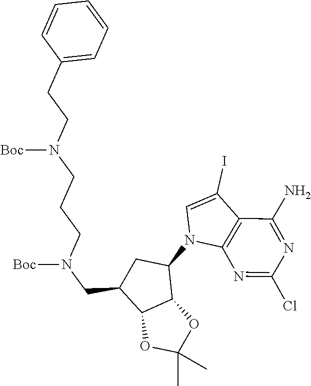 [SMILES: CC(C)(C)OC(=O)N(CCCN(C[C@H]1C[C@@H](n2cc(I)c3c(N)nc(Cl)nc32)[C@@H]2OC(C)(C)O[C@H]12)C(=O)OC(C)(C)C)CCc1ccccc1]

To tert-butyl-N-[3-({[(3aR,4R,6R,6aS)-6-{2,4-dichloro-5-iodopyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 308c (0.10 g, 0.12 mmol, 1.0 eq) was added 7M NH₃ in MeOH (2.0 mL) at room temperature and the reaction mixture was stirred at 80° C. for 8 h. The resulting mixture was concentrated under vacuum and the crude residue was purified by silica gel column chromatography (1:1 tetrahydrofuran: Petroleum ether) to afford tert-butyl-N-[3-({[(3aR,4R,6R,6aS)-6-{4-amino-2-chloro-5-iodopyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 309c (88 mg, 90%) as an off white solid. LCMS [conditions Cortecs C18+, 50*3.0 mm, 2.7 μm, Mobile Phase A: Water/0.05% TFA, Mobile Phase B: Acetonitrile, 2%-95% B-3 min (+), 1.50 mL/min]: T_{R}=1.89 min; [M+H]⁺: 825, 827.

### tert-Butyl-(3-((((3aR,4R,6R,6aS)-6-(4-amino-5-(4-benzylthiazol-2-yl)-2-chloro-7H-pyrrolo[2,3-d]pyrimidin-7-yl)-2,2-dimethyltetrahydro-4H-cyclopenta[d][1,3]dioxol-4-yl)methyl)(tert-butoxycarbonyl)amino)propyl)(phenethyl)carbamate (310c)

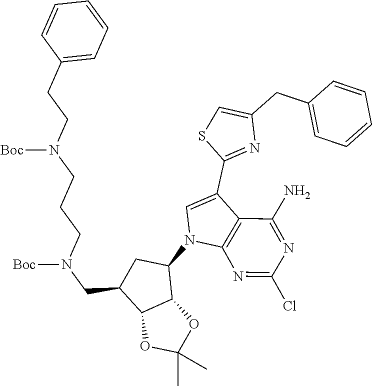 [SMILES: CC(C)(C)OC(=O)N(CCCN(C[C@H]1C[C@@H](n2cc(-c3nc(Cc4ccccc4)cs3)c3c(N)nc(Cl)nc32)[C@@H]2OC(C)(C)O[C@H]12)C(=O)OC(C)(C)C)CCc1ccccc1]

To a solution of tert-butyl-N-[3-({[(3aR,4R,6R,6aS)-6-{4-amino-2-chloro-5-iodopyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 309c (88 mg, 0.11 mmol, 1.0 eq) and 4-benzyl-2-(tributylstannyl)-1,3-thiazole (0.25 g, 0.50 mmol, 4.5 eq) [WO2011105590]in toluene (2.0 mL) was added Pd (PPh₃) 2Cl₂ (7.5 mg, 0.01 mmol, 0.1 eq) and the reaction mixture was stirred at 100° C. for 4 h under N₂. The resulting mixture was concentrated under vacuum and the crude residue was purified by silica gel column chromatography (2:1 tetrahydrofuran: Petroleum ether) to afford tert-butyl-(3-((((3aR,4R,6R,6aS)-6-(4-amino-5-(4-benzylthiazol-2-yl)-2-chloro-7H-pyrrolo[2,3-d]pyrimidin-7-yl)-2,2-dimethyltetrahydro-4H-cyclopenta[d][1,3]dioxol-4-yl)methyl)(tert-butoxycarbonyl)amino)propyl)(phenethyl)carbamate 310c (72 mg, 77%) as an off white solid. LCMS [conditions Cortecs C18+, 50*3.0 mm, 2.7 μm, Mobile Phase A: Water/0.05% TFA, Mobile Phase B: Acetonitrile, 2%-95% B-3 min (+), 1.50 mL/min]: T_{R}=2.15 min; [M+H]⁺: 872, 874.

### (1R,2S,3R,5R)-3-(4-Amino-5-(4-benzylthiazol-2-yl)-2-chloro-7H-pyrrolo[2,3-d]pyrimidin-7-yl)-5-(((3-(phenethylamino)propyl)amino)methyl)cyclopentane-1,2-diol (635)

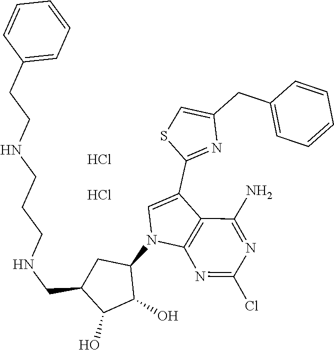 [SMILES: Nc1nc(Cl)nc2c1c(-c1nc(Cc3ccccc3)cs1)cn2[C@@H]1C[C@H](CNCCCNCCc2ccccc2)[C@@H](O)[C@H]1O ; Cl ; Cl]

To tert-butyl-N-[3-({[(3aR,4R,6R,6aS)-6-[4-amino-5-(4-benzyl-1,3-thiazol-2-yl)-2-chloropyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate 309c (72.0 mg, 0.08 mmol, 1.0 eq) was added 4M HCl in MeOH (2.0 mL, 8.00 mmol) at room temperature and the reaction mixture was stirred at 50° C. for 0.5 h. The precipitated solids were collected by filtration, washed with n-hexanes (3×10 mL) and the solid was dried under vacuum to afford (1R,2S,3R,5R)-3-(4-amino-5-(4-benzylthiazol-2-yl)-2-chloro-7H-pyrrolo[2,3-d]pyrimidin-7-yl)-5-(((3-(phenethylamino)propyl)amino)methyl)cyclopentane-1,2-diol dihydrochloride 635 (20.2 mg, 39%) as a white solid. LCMS [conditions ZORBAX SB-Aq, 50*4.6 mm, 1.8 μm, Mobile Phase A: Water/0.02% TFA, Mobile Phase B: Acetonitrile, 5%-95% B-3 min-1.5.lcm, 1.50 mL/min, ES, m/z]: T_{R}=1.36 min; [M+H]⁺: 632, 634. ¹H NMR (300 MHZ, DMSO-d₆+D₂O) δ 8.14 (s, 1H), 7.42-7.18 (m, 11H), 4.89-4.74 (m, 1H), 4.30-4.21 (m, 1H), 4.06 (s, 2H), 3.91 (t, J=5.1 Hz, 1H), 3.22-2.91 (m, 10H), 2.38-2.22 (m, 2H), 2.12-1.98 (m, 2H), 1.69-1.53 (m, 1H).

### Synthesis of (1R,2S,3R,5R)-3-(4-amino-5-(1-benzyl-1H-pyrazol-3-yl)-2-chloro-7H-pyrrolo[2,3-d]pyrimidin-7-yl)-5-(((3-(phenethylamino)propyl)amino)methyl)cyclopentane-1,2-diol (636)

### tert-Butyl-(3-((((3aR,4R,6R,6aS)-6-(4-amino-5-(1-benzyl-1H-pyrazol-3-yl)-2-chloro-7H-pyrrolo[2,3-d]pyrimidin-7-yl)-2,2-dimethyltetrahydro-4H-cyclopenta[d][1,3]dioxol-4-yl)methyl)(tert-butoxycarbonyl)amino)propyl)(phenethyl)carbamate (311r)

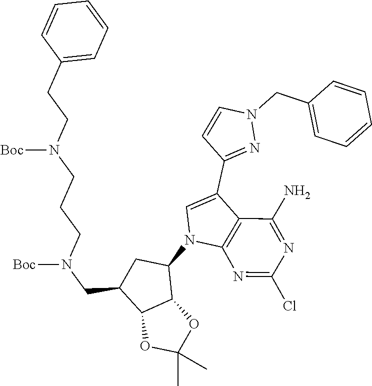 [SMILES: CC(C)(C)OC(=O)N(CCCN(C[C@H]1C[C@@H](n2cc(-c3ccn(Cc4ccccc4)n3)c3c(N)nc(Cl)nc32)[C@@H]2OC(C)(C)O[C@H]12)C(=O)OC(C)(C)C)CCc1ccccc1]

To a solution of tert-butyl-NV-[3-({[(3aR,4R,6R,6aS)-6-{4-amino-2-chloro-5-iodopyrrolo[2,3-d]pyrimidin-7-yl}-2,2-dimethyl-tetrahydro-3aH-cyclopenta[d][1,3]dioxol-4-yl]methyl}(tert-butoxycarbonyl)amino)propyl]-N-(2-phenylethyl)carbamate 309c (80.0 mg, 0.10 mmol, 1.0 eq) and 1-benzylpyrazol-3-ylboronic acid (29.4 mg, 0.15 mmol, 1.5 eq) [WO2017040450]in dioxane (1.5 mL) and H₂O (0.15 mL) were added K₃PO₄ (61.7 mg, 0.30 mmol, 3.0 eq) and Pd (DtBPF) Cl₂ (6.3 mg, 0.01 mmol, 0.1 eq) and the reaction mixture was stirred at 60° C. for 0.5 h, under N₂. The reaction was quenched with water (10 mL) and the resulting mixture was extracted with EtOAc (3×10 mL). The combined organic extracts were dried over anhydrous Na₂SO₄ and the filtrate was concentrated under reduced pressure. The crude residue was purified by silica gel column chromatography (2:1 tetrahydrofuran: petroleum ether) to afford tert-butyl-N-[3-({[(3aR,4R,6R,6aS)-6-[4-amino-5-(1-benzylpyrazol-3-yl)-2-chloropyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate 311c (53.0 mg, 64%) as an off white solid. LCMS [conditions Cortecs C18+, 50*3.0 mm, 2.7 μm, Mobile Phase A: Water/0.05% TFA, Mobile Phase B: Acetonitrile, 2%-95% B-3 min (+)(90-2000).lcm, 1.50 mL/min, ES, m/z]: T_{R}=2.22 min; [M+H]⁺: 855, 857.

### (1R,2S,3R,5R)-3-(4-Amino-5-(1-benzyl-1H-pyrazol-3-yl)-2-chloro-7H-pyrrolo[2,3-d]pyrimidin-7-yl)-5-(((3-(phenethylamino)propyl)amino)methyl)cyclopentane-1,2-diol (636)

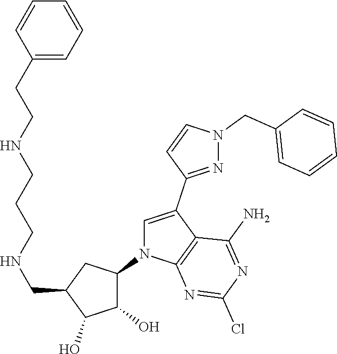 [SMILES: Nc1nc(Cl)nc2c1c(-c1ccn(Cc3ccccc3)n1)cn2[C@@H]1C[C@H](CNCCCNCCc2ccccc2)[C@@H](O)[C@H]1O]

To tert-butyl-N-[3-({[(3aR,4R,6R,6aS)-6-[4-amino-5-(1-benzylpyrazol-3-yl)-2-chloropyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate 311c (53.0 mg, 0.06 mmol, 1.0 eq) was added 4M HCl in MeOH (2.0 mL, 8.00 mmol) and the reaction mixture was stirred at 50° C. for 1 h. The resulting mixture was concentrated under reduced pressure, dissolved in DMF (3.0 mL) and the solution was adjusted to pH 8 with Na₂CO₃. The crude residue was purified by Prep-HPLC [Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 25% B to 65% B in 7 min, 55% B; Wave Length: 220 nm]: to afford (1R,2S,3R,5R)-3-[4-amino-5-(1-benzylpyrazol-3-yl)-2-chloropyrrolo[2,3-d]pyrimidin-7-yl]-5-[({3-[(2-phenylethyl)amino]propyl}amino)methyl]cyclopentane-1,2-diol 636 (15.1 mg, 40%) as a white solid. LCMS [conditions ZORBAX SB-Aq, 50*4.6 mm, 1.8 μm, Mobile Phase A: Water/0.02% TFA, Mobile Phase B: Acetonitrile, 5%-95% B-3 min-1.5.lcm, 1.50 mL/min, ES, m/z]: T_{R}=1.36 min; [M+H]⁺: 615, 617. ¹H NMR (400 MHZ, DMSO-d₆) δ 9.21 (s, 1H), 7.89 (s, 1H), 7.86 (s, 1H), 7.60 (s, 1H), 7.39-7.15 (m, 10H), 6.70 (d, J=2.3 Hz, 1H), 5.37 (s, 2H), 4.87-4.77 (m, 2H), 4.16 (t, J=7.1 Hz, 1H), 3.77 (t, J=4.5 Hz, 1H), 2.79-2.55 (m, 10H), 2.21-2.13 (m, 1H), 2.08-2.01 (m, 1H), 1.59-1.52 (m, 2H), 1.49-1.40 (m, 1H).

### Synthesis of (1R,2S,3R,5R)-3-(5-(4-benzylthiophen-2-yl)-7H-pyrrolo[2,3-d]pyrimidin-7-yl)-5-(((3-(phenethylamino)propyl)amino)methyl)cyclopentane-1,2-diol (637)

### (E)-N-((5-Bromothiophen-3-yl)methylene)-4-methylbenzenesulfonohvdrazide (313c)

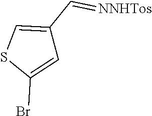

To a solution of 5-bromothiophene-3-carbaldehyde (0.50 g, 2.60 mmol, 1.0 eq) in dioxane (5.0 mL) was added 4-toluenesulfonyl hydrazide (0.49 g, 2.60 mmol, 1.0 eq) at room temperature and the reaction mixture was stirred for 1 h at room temperature. The resulting mixture was concentrated under reduced pressure to afford N-[(12)-(5-bromothiophen-3-yl)methylidene]-4-methylbenzenesulfonohydrazide 313c (0.80 g, 85%) was used in the next step directly without purification. LCMS [L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-2 min (+)]: T_{R}=1.16 min; [M+H]⁺: 359, 361.

### 4-Benzyl-2-bromothiophene (314c)

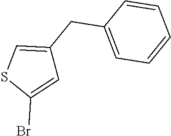 [SMILES: Brc1cc(Cc2ccccc2)cs1]

To a solution of (E)-N-((5-bromothiophen-3-yl)methylene)-4-methylbenzenesulfonohydrazide 313c (0.80 g, 2.20 mmol, 1.0 eq) and phenylboronic acid (0.54 g, 4.45 mmol, 2.0 eq) in dioxane (10 mL) was added Cs₂CO₃ (2.18 g, 6.70 mmol, 3.0 eq) and the reaction mixture was stirred at 100° C. for 2 h, under N₂. The resulting mixture was concentrated under reduced pressure and the residue was partitioned between ethyl acetate and brine. The organic layer was dried over anhydrous Na₂SO₄ and the filtrate was concentrated under reduced pressure. The crude residue was purified by silica gel column chromatography (1:5 ethyl acetate:petroleum ether) to afford 4-benzyl-2-bromothiophene 314c (0.45 g, 80%) as a pale yellow oil. LCMS [L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-2 min (+)]: T_{R}=1.69 min; [M+H]⁺: 253, 255.

### 2-(4-Benzylthiophen-2-yl)-4,4,5,5-tetramethyl-1,3,2-dioxaborolane (315c)

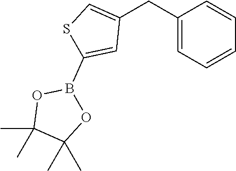 [SMILES: CC1(C)OB(c2cc(Cc3ccccc3)cs2)OC1(C)C]

To a solution of 4-benzyl-2-bromothiophene 314c (0.20 g, 0.80 mmol, 1.0 eq), bis(pinacolato)diboron (0.30 g, 1.20 mmol, 1.5 eq) and AcOK (0.23 g, 2.40 mmol, 3.0 eq) in DMF (3.0 mL) was added Pd (dppf) Cl₂ (57.8 mg, 0.08 mmol, 0.1 eq) and the reaction mixture was stirred at 100° C. for 2 h, under N₂. The resulting mixture was allowed to cool to room temperature and was partitioned between AcOEt and brine. The organic layer was dried over anhydrous Na₂SO₄ and the filtrate was concentrated under reduced pressure to afford 2-(4-benzylthiophen-2-yl)-4,4,5,5-tetramethyl-1,3,2-dioxaborolane 315c (200 mg, crude) which was used in the next step directly without purification.

### tert-Butyl-(((3aR,4R,6R,6aS)-6-(5-(4-benzylthiophen-2-yl)-7H-pyrrolo[2,3-d]pyrimidin-7-yl)-2,2-dimethyltetrahydro-4H-cyclopenta[d][1,3]dioxol-4-yl)methyl)(3-((tert-butoxycarbonyl)(phenethyl)amino)propyl)carbamate (316c)

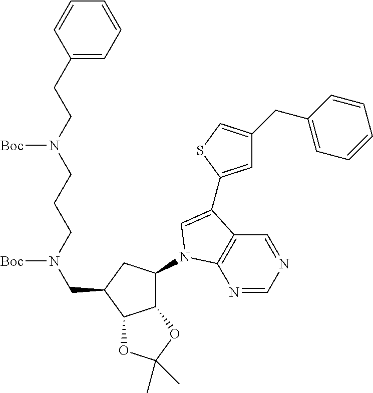 [SMILES: CC(C)(C)OC(=O)N(CCCN(C[C@H]1C[C@@H](n2cc(-c3cc(Cc4ccccc4)cs3)c3cncnc32)[C@@H]2OC(C)(C)O[C@H]12)C(=O)OC(C)(C)C)CCc1ccccc1]

To a solution of tert-butyl-(((3aR,4R,6R,6aS)-6-(5-bromo-7H-pyrrolo[2,3-d]pyrimidin-7-yl)-2,2-dimethyltetrahydro-4H-cyclopenta[d][1,3]dioxol-4-yl)methyl)(3-((tert-butoxycarbonyl)(phenethyl)amino)propyl)carbamate 220c (40.0 mg, 0.06 mmol, 1.0 eq) and 2-(4-benzylthiophen-2-yl)-4,4,5,5-tetramethyl-1,3,2-dioxaborolane 315c (49.4 mg, 0.17 mmol, 3.0 eq) in dioxane (1.5 mL) and H₂O (0.15 mL) were added K₃PO₄ (35.0 mg, 0.17 mmol, 3.0 eq) and Pd (dtbpf) Cl₂ (3.6 mg, 0.006 mmol, 0.1 eq) and the reaction mixture was stirred at 90° C. for 0.5 h, under N₂. The reaction was quenched with water (10 mL) and then extracted with EtOAc (3×10 mL). The combined organic extracts were dried over anhydrous Na₂SO₄ and the filtrate was concentrated under reduced pressure. The crude residue was purified by silica gel column chromatography (3:7 tetrahydrofuran: petroleum ether) to afford tert-butyl-(((3aR,4R,6R,6aS)-6-(5-(4-benzylthiophen-2-yl)-7H-pyrrolo[2,3-d]pyrimidin-7-yl)-2,2-dimethyltetrahydro-4H-cyclopenta[d][1,3]dioxol-4-yl)methyl)(3-((tert-butoxycarbonyl)(phenethyl)amino)propyl)carbamate 316c (40.0 mg, 88%) as a yellow oil. LCMS [L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min (+), 1.50 mL/min]: T_{R}=2.77 min; [M+H]⁺: 822.

### (1R,2S,3R,5R)-3-(5-(4-Benzylthiophen-2-yl)-7H-pyrrolo[2,3-d]pyrimidin-7-yl)-5-(((3-(phenethylamino)propyl)amino)methyl)cyclopentane-1,2-diol (637)

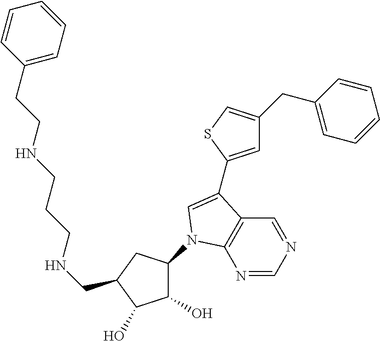 [SMILES: O[C@@H]1[C@@H](CNCCCNCCc2ccccc2)C[C@@H](n2cc(-c3cc(Cc4ccccc4)cs3)c3cncnc32)[C@@H]1O]

To a solution of tert-butyl-N-[3-({[(3aR,4R,6R,6aS)-6-[5-(4-benzylthiophen-2-yl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate 316c (40.0 mg, 0.05 mmol, 1.0 eq) in MeOH (0.5 mL) was added 4 M HCl in MeOH (2.0 mL, 8.00 mmol) at room temperature and the reaction mixture was stirred at 50° C. for 0.5 h. The resulting mixture was concentrated under reduced pressure, dissolved in MeOH (3.0 mL) and adjusted to pH 8 with Na₂CO₃. The crude residue was purified by Prep-HPLC [Column: YMC Triart C18, 30*150 mm, 5 μm; Mobile Phase A: 20 mM NH₄HCO₃+0.05% NH₃H₂O, Mobile Phase B: ACN; Flow rate: 35 mL/min; Gradient: 10% to 65% in 8 min]: to afford (1R,2S,3R,5R)-3-[5-(4-benzylthiophen-2-yl)pyrrolo[2,3-d]pyrimidin-7-yl]-5-[{{3-[(2-phenylethyl)amino]propyl}amino)methyl]cyclopentane-1,2-diol 637 (20 mg, 70%) as a white solid. LCMS [conditions Cortecs C18+, 50*3.0 mm, 2.7 μm, Mobile Phase A: Water/0.02% TFA, Mobile Phase B: Acetonitrile, 5%-95% B-3 min-1.5.lcm, 1.50 mL/min]: T_{R}=0.92 min; [M+H]⁺: 582.1H NMR (400 MHZ, DMSO-d₆+D₂O) δ 9.23 (s, 1H), 8.83 (s, 1H), 8.13 (s, 1H), 7.37-7.28 (m, 5H), 7.26-7.18 (m, 3H), 7.17-7.08 (m, 4H), 5.14-4.97 (m, 1H), 4.27 (dd, J=8.8, 5.6 Hz, 1H), 3.96 (s, 2H), 3.79 (dd, J=5.6, 3.4 Hz, 1H), 2.74-2.52 (m, 10H), 2.30-2.15 (m, 1H), 2.14-2.03 (m, 1H), 1.66-1.48 (m, 3H).

### Synthesis of (1R,2S,3R,5R)-3-(2-chloro-5-(thiazol-2-yl)-7H-pyrrolo[2,3-d]pyrimidin-7-yl)-5-(((3-(phenethylamino)propyl)amino)methyl)cyclopentane-1,2-diol (638)

### 2-(2-Chloro-7H-pyrrolo[2,3-d]pyrimidin-5-yl) thiazole (317c)

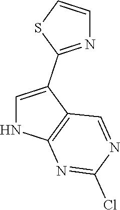 [SMILES: Clc1ncc2c(-c3nccs3)c[nH]c2n1]

To a solution of 2-chloro-5-iodo-7H-pyrrolo[2,3-d]pyrimidine (0.20 g, 0.70 mmol, 1.0 eq) and 2-(tributylstannyl)-1,3-thiazole (0.54 g, 1.40 mmol, 2.0 eq) in toluene (4.0 mL) was added Pd (PPh₃) 2Cl₂ (50.2 mg, 0.07 mmol, 0.1 eq) and the reaction mixture was stirred at 110° C. for 12 h, under N₂. The reaction was quenched with water (10 mL) and then extracted with EtOAc (3×10 mL). The combined organic extracts were dried over anhydrous Na₂SO₄ and the filtrate was concentrated under reduced pressure. The crude residue was purified by Prep-HPLC [Column: YMC-Actus Triart C18, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 15% B to 55% B in 7 min, 55% B; Wave Length: 220 nm]: to afford 2-{2-chloro-7H-pyrrolo[2,3-d]pyrimidin-5-yl}-1,3-thiazole 317 (20 mg, 12%) as a white solid. LCMS [L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-2 min (+)]: T_{R}=1.28 min; [M+H]⁺: 237, 239.

### tert-Butyl-(3-((tert-butoxycarbonyl)(((3aR,4R,6R,6aS)-6-(2-chloro-5-(thiazol-2-yl)-7H-pyrrolo[2,3-d]pyrimidin-7-yl)-2,2-dimethyltetrahydro-4H-cyclopenta[d][1,3]dioxol-4-yl)methyl)amino)propyl)(phenethyl)carbamate (318c)

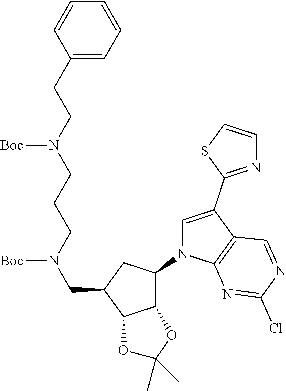 [SMILES: CC(C)(C)OC(=O)N(CCCN(C[C@H]1C[C@@H](n2cc(-c3nccs3)c3cnc(Cl)nc32)[C@@H]2OC(C)(C)O[C@H]12)C(=O)OC(C)(C)C)CCc1ccccc1]

To a stirred solution of 2-{2-chloro-7H-pyrrolo[2,3-d]pyrimidin-5-yl}-1,3-thiazole 317c (36.0 mg, 0.15 mmol, 1.0 eq), tert-butyl-(3-((tert-butoxycarbonyl)(((3aR,4R,6S,6aS)-6-hydroxy-2,2-dimethyltetrahydro-4H-cyclopenta[d][1,3]dioxol-4-yl)methyl)amino)propyl)(phenethyl)carbamate 256c (0.11 g, 0.20 mmol, 1.3 eq) and PPh₃ (79.8 mg, 0.30 mmol, 2.0 eq) in toluene (1.0 mL) was added a solution of DBAD (70.0 mg, 0.30 mmol, 2.0 eq) in toluene (1.0 mL), dropwise at 0° C., and the reaction mixture was stirred at 60° C. for 4 h. The resulting mixture was concentrated under reduced pressure, dissolved in DMF (2.0 mL) and the crude residue was purified by reverse flash chromatography (Column, C18 silica gel; mobile phase, Water/0.05% TFA in acetonitrile, 30% to 85% gradient in 15 min; detector, UV 254 nm) to afford tert-butyl-(3-((tert-butoxycarbonyl)(((3aR,4R,6R,6aS)-6-(2-chloro-5-(thiazol-2-yl)-7H-pyrrolo[2,3-d]pyrimidin-7-yl)-2,2-dimethyltetrahydro-4H-cyclopenta[d][1,3]dioxol-4-yl)methyl)amino)propyl)(phenethyl)carbamate 318c (38.0 mg, 32%) as an off white solid. LCMS [conditions Cortecs C18+, 50*3.0 mm, 2.7 μm, Mobile Phase A: Water/0.05% TFA, Mobile Phase B: Acetonitrile, 2%-95% B-3 min (+)(90-2000).lcm, 1.50 mL/min, ES, m/z]: T_{R}=2.24 min; [M+H]⁺: 767, 769.

### (1R,2S,3R,5R)-3-(2-Chloro-5-(thiazol-2-yl)-7H-pyrrolo[2,3-d]pyrimidin-7-yl)-5-(((3-(phenethylamino)propyl)amino)methyl)cyclopentane-1,2-diol (638)

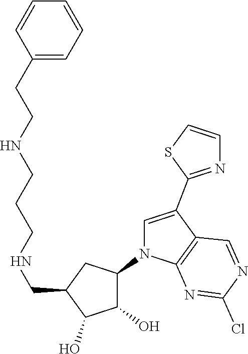 [SMILES: O[C@@H]1[C@@H](CNCCCNCCc2ccccc2)C[C@@H](n2cc(-c3nccs3)c3cnc(Cl)nc32)[C@@H]1O]

To tert-butyl-(3-((tert-butoxycarbonyl)(((3aR,4R,6R,6aS)-6-(2-chloro-5-(thiazol-2-yl)-7H-pyrrolo[2,3-d]pyrimidin-7-yl)-2,2-dimethyltetrahydro-4H-cyclopenta[d][1,3]dioxol-4-yl)methyl)amino)propyl)(phenethyl)carbamate 318c (36.0 mg, 0.05 mmol, 1.0 eq) was added 4 M HCl in MeOH (1.0 mL, 4.00 mmol) at room temperature and the reaction mixture was stirred at 50° C. for 0.5 h. The resulting mixture was concentrated under reduced pressure, dissolved in MeOH (3.0 mL) and the solution was adjusted to pH 8 with Na₂CO₃. The crude residue was purified by Prep-HPLC [Column: YMC-Actus Triart C18, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 25% B to 65% B in 7 min, 65% B; Wave Length: 220 nm]: to afford (1R,2S,3R,5R)-3-(2-chloro-5-(thiazol-2-yl)-7H-pyrrolo[2,3-d]pyrimidin-7-yl)-5-(((3-(phenethylamino)propyl)amino)methyl)cyclopentane-1,2-diol 638 (5.2 mg, 21%) as a white solid. LCMS [conditions ZORBAX SB-Aq, 50*4.6 mm, 1.8 μm, Mobile Phase A: Water/0.02% TFA, Mobile Phase B: Acetonitrile, 5%-95% B-3 min-1.5.lcm, 1.50 mL/min, ES, m/z]: T_{R}=1.22 min; [M+H]⁺: 527, 529. ¹H NMR (400 MHZ, DMSO-d₆) δ 9.37 (s, 1H), 8.65 (s, 1H), 7.93 (d, J=3.3 Hz, 1H), 7.73 (d, J=3.3 Hz, 1H), 7.30-7.22 (m, 2H), 7.21-7.11 (m, 3H), 5.06-4.91 (m, 2H), 4.36-4.28 (m, 1H), 3.83-3.78 (m, 1H), 2.80-2.57 (m, 10H), 2.28-2.24 (m, 1H), 2.11-2.06 (m, 1H), 1.68-1.56 (m, 3H).

### Synthesis of (15,2R,3R,5R)-3-(((3-(phenethylamino)propyl)amino)methyl)-5-(5-(4-phenoxythiazol-2-yl)-7H-pyrrolo[2,3-d]pyrimidin-7-yl)cyclopentane-1,2-diol (639)

### tert-Butyl-(3-((tert-butoxycarbonyl)(((3aR,4R,6R,6aS)-2,2-dimethyl-6-(5-(4-phenoxythiazol-2-yl)-7H-pyrrolo[2,3-d]pyrimidin-7-yl)tetrahydro-4H-cyclopenta[d][1,3]dioxol-4-yl)methyl)amino)propyl)(phenethyl)carbamate (319c)

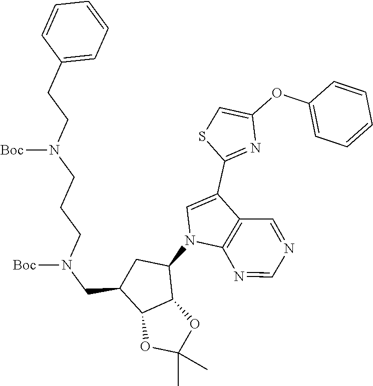 [SMILES: CC(C)(C)OC(=O)N(CCCN(C[C@H]1C[C@@H](n2cc(-c3nc(Oc4ccccc4)cs3)c3cncnc32)[C@@H]2OC(C)(C)O[C@H]12)C(=O)OC(C)(C)C)CCc1ccccc1]

To a solution of tert-butyl-(3-((tert-butoxycarbonyl)(phenethyl)amino)propyl)(((3aR,4R,6R,6aS)-6-(5-iodo-7H-pyrrolo[2,3-d]pyrimidin-7-yl)-2,2-dimethyltetrahydro-4H-cyclopenta[d][1,3]dioxol-4-yl)methyl)carbamate 226c (0.18 g, 0.39 mmol, 1.0 eq) and 4-phenoxy-2-(tributylstannyl)-1,3-thiazole 208c (0.182 g, 0.39 mmol, 1.0 eq) in toluene (2.0 mL) was added Pd (PPh₃) 2Cl₂ (27.3 mg, 0.039 mmol, 0.1 eq) and the reaction mixture was stirred at 100° C. for 4 h, under N₂. The reaction was quenched with water (10 mL) and extracted with EtOAc (3×10 mL). The combined organic extracts were dried over anhydrous Na₂SO₄ and the filtrate was concentrated under reduced pressure. The crude residue was purified by silica gel column chromatography (1:2 tetrahydrofuran: petroleum ether) to afford tert-butyl-(3-((tert-butoxycarbonyl)(((3aR,4R,6R,6aS)-2,2-dimethyl-6-(5-(4-phenoxythiazol-2-yl)-7H-pyrrolo[2,3-d]pyrimidin-7-yl)tetrahydro-4H-cyclopenta[d][1,3]dioxol-4-yl)methyl)amino)propyl)(phenethyl)carbamate 319c (76 mg, 71%) as a light yellow oil. LCMS [Column: Kinetex XB-C18, 50*3.0 mm, 2.6 μm, Mobile Phase A: Water/0.05% TFA, Mobile Phase B: Acetonitrile, 2%-100% B-2 min (+).Icm, 1.50 mL/min, ES, m/z]: T_{R}=1.53 min; [M+H]⁺: 825.

### (1S,2R,3R,5R)-3-(((3-(Phenethylamino)propyl)amino)methyl)-5-(5-(4-phenoxythiazol-2-yl)-7H-pyrrolo[2,3-d]pyrimidin-7-yl)cyclopentane-1,2-diol (639)

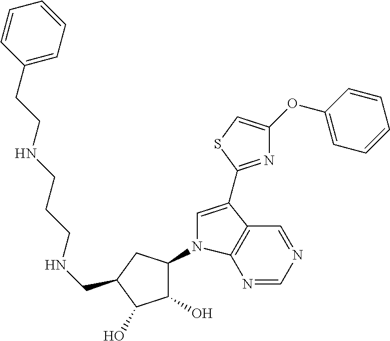 [SMILES: O[C@@H]1[C@@H](CNCCCNCCc2ccccc2)C[C@@H](n2cc(-c3nc(Oc4ccccc4)cs3)c3cncnc32)[C@@H]1O]

To tert-butyl-(3-((tert-butoxycarbonyl)(((3aR,4R,6R,6aS)-2,2-dimethyl-6-(5-(4-phenoxythiazol-2-yl)-7H-pyrrolo[2,3-d]pyrimidin-7-yl)tetrahydro-4H-cyclopenta[d][1,3]dioxol-4-yl)methyl)amino)propyl)(phenethyl)carbamate 319c (70.0 mg, 0.09 mmol, 1.0 eq) was added 4M HCl in MeOH (2.0 mL, 8.00 mmol) at room temperature and the reaction mixture was stirred at 50° C. for 0.5 h. The resulting mixture was concentrated under reduced pressure, dissolved in DMF (3.0 mL) and the solution was adjusted to pH 8 with NH₃·H₂O. The crude residue was purified by Prep-HPLC [Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 20% B to 65% B in 9 min, 55% B; Wave Length: 220 nm]: to afford (1S,2R,3R,5R)-3-(((3-(phenethylamino)propyl)amino)methyl)-5-(5-(4-phenoxythiazol-2-yl)-7H-pyrrolo[2,3-d]pyrimidin-7-yl)cyclopentane-1,2-diol 639 (20.7 mg, 42%) as a white solid. LCMS [conditions ZORBAX SB-Aq, 50*4.6 mm, 1.8 μm, Mobile Phase A: Water/0.02% TFA, Mobile Phase B: MeOH, 5%-95% B-5 min-1.0-AB.Icm, 1.50 mL/min, ES, m/z]: T_{R}=1.33 min; [M+H]⁺: 585. ¹H NMR (400 MHZ, DMSO-d₆) δ 9.31 (s, 1H), 8.90 (s, 1H), 8.61 (s, 1H), 7.49-7.40 (m, 2H), 7.31-7.12 (m, 8H), 6.84 (s, 1H), 5.09 (dd, J=10.6, 8.4 Hz, 1H), 4.89 (br s, 1H), 4.42-4.30 (m, 1H), 3.87-3.75 (m, 1H), 2.95-2.54 (m, 10H), 2.33-2.20 (m, 1H), 2.15-2.02 (m, 1H), 1.74-1.51 (m, 3H).

### Synthesis of (1R,2S,3R,5R)-3-(5-(1-(methylsulfonyl)-1H-pyrazol-3-yl)-7H-pyrrolo[2,3-d]pyrimidin-7-yl)-5-(((3-(phenethylamino)propyl)amino)methyl)cyclopentane-1,2-diol (640)

### tert-Butyl-(3-((tert-butoxycarbonyl)(((3aR,4R,6R,6aS)-2,2-dimethyl-6-(5-(1-(methylsulfonyl)-1H-pyrazol-3-yl)-7H-pyrrolo[2,3-d]pyrimidin-7-yl)tetrahydro-4H-cyclopenta[d][1,3]dioxol-4-yl)methyl)amino)propyl)(phenethyl)carbamate (323c)

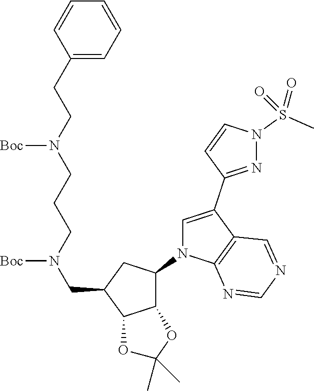 [SMILES: CC(C)(C)OC(=O)N(CCCN(C[C@H]1C[C@@H](n2cc(-c3ccn(S(C)(=O)=O)n3)c3cncnc32)[C@@H]2OC(C)(C)O[C@H]12)C(=O)OC(C)(C)C)CCc1ccccc1]

To a solution of tert-butyl-(((3aR,4R,6R,6aS)-6-(5-bromo-7H-pyrrolo[2,3-d]pyrimidin-7-yl)-2,2-dimethyltetrahydro-4H-cyclopenta[d][1,3]dioxol-4-yl)methyl)(3-((tert-butoxycarbonyl)(phenethyl)amino)propyl)carbamate 220c (0.10 g, 0.14 mmol, 1.0 eq) and 1-methanesulfonyl-3-(4,4,5,5-tetramethyl-1,3,2-dioxaborolan-2-yl)pyrazole 200c (74.7 mg, 0.30 mmol, 2.0 eq) and K₃PO₄ (87.4 mg, 0.40 mmol, 3.0 eq) in dioxane (1.5 mL) and water (0.15 mL), under N₂, was added Pd (DtBPF) Cl₂ (8.9 mg, 0.014 mmol, 0.1 eq) and the reaction mixture was stirred at 90° C. for 0.5 h. The reaction was quenched with water (10 mL) and then extracted with EtOAc (3×10 mL). The combined organic extracts were dried over anhydrous Na₂SO₄, filtered and the filtrate was concentrated under reduced pressure. The crude residue was purified by silica gel column chromatography (1:1 tetrahydrofuran: petroleum ether) to afford tert-butyl-N-[3-({[(3aR,4R,6R,6aS)-6-[5-(1-methanesulfonylpyrazol-3-yl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate 323c (88.0 mg, 81%) as a yellow oil. LCMS [Column: Kinetex XB-C18, 50*3.0 mm, 2.6 μm, Mobile Phase A: Water/0.05% TFA, Mobile Phase B: Acetonitrile, 2%-100% B-2 min (+).Icm, 1.50 mL/min, ES, m/z]: T_{R}=1.86 min; [M+H]⁺: 794.

### (1R,2S,3R,5R)-3-(5-(1-(Methylsulfonyl)-1H-pyrazol-3-yl)-7H-pyrrolo[2,3-d]pyrimidin-7-yl)-5-(((3-(phenethylamino)propyl)amino)methyl)cyclopentane-1,2-diol (640)/>

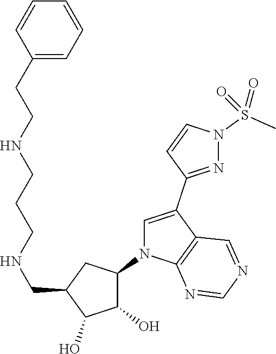 [SMILES: CS(=O)(=O)n1ccc(-c2cn([C@@H]3C[C@H](CNCCCNCCc4ccccc4)[C@@H](O)[C@H]3O)c3ncncc23)n1]

To tert-butyl-N-[3-({3aR,4R,6R,6aS)-6-15-(1-methanesulfonylpyrazol-3-yl)pyrrolo[2,3-d]pyrimidin-7-yl]-2,2-dimethyl-tetrahydro-3aH-cyclopenta[d][1,3]dioxol-4-yl]methyl}(tert-butoxycarbonyl)amino)propyl]-N-(2-phenylethyl)carbamate 640 (82.0 mg, 0.10 mmol, 1.0 eq) in DCM (1.0 mL) was added TFA (1.0 mL) and the reaction mixture was stirred at 50° C. for 1 h. The resulting mixture was concentrated under reduced pressure, dissolved in DMF (3.0 mL) and the solution was adjusted to pH 8 with NH₃·H₂O. The crude residue was purified by Prep-HPLC [Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 25% B to 65% B in 9 min, 55% B; Wave Length: 220 nm]: to afford (1R,2S,3R,5R)-3-[5-(1-methanesulfonylpyrazol-3-yl)pyrrolo[2,3-d]pyrimidin-7-yl]-5-[{{3-[(2-phenylethyl)amino]propyl}amino)methyl]cyclopentane-1,2-diol 640 (26.5 mg, 46%) as a white solid. LCMS [conditions ZORBAX SB-Aq, 50*4.6 mm, 1.8 μm, Mobile Phase A: Water/0.02% TFA, Mobile Phase B: MeOH, 5%-95% B-5 min-1.0-AB.Icm, 1.50 mL/min, ES, m/z]: T_{R}=1.11 min; [M+H]⁺: 554.1H NMR (400 MHZ, DMSO-d₆+D₂O) δ 9.49 (s, 1H), 8.89 (s, 1H), 8.47 (s, 1H), 8.35 (d, J=2.8 Hz, 1H), 7.29-7.22 (m, 2H), 7.21-7.12 (m, 3H), 7.10 (d, J=2.8 Hz, 1H), 5.08 (dd, J=10.6, 8.4 Hz, 1H), 4.99-4.84 (m, 1H), 4.37-4.23 (m, 1H), 3.89-3.80 (m, 1H), 3.64 (s, 3H), 2.84-2.55 (m, 10H), 2.38-2.24 (m, 1H), 2.15-2.02 (m, 1H), 1.67-1.51 (m, 3H).

### Synthesis of (1R,2S,3R,5R)-3-(2-chloro-5-(thiophen-2-yl)-7H-pyrrolo[2,3-d]pyrimidin-7-yl)-5-(((3-(phenethylamino)propyl)(1H-pyrazol-4-yl)amino)methyl)cyclopentane-1,2-diol (641)

### (3aR,6R,6aR)-6-(((tert-Butyldiphenylsilyl)oxy)methyl)-2,2-dimethyltetrahydro-4H-cyclopenta[d][1,3]dioxol-4-one (324c)

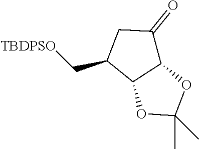

To a solution of (3aR,6R,6aR)-6-(hydroxymethyl)-2,2-dimethyl-tetrahydrocyclopenta[d][1,3]dioxol-4-one (1.00 g, 5.37 mmol, 1.0 eq) [Bioorg. Med. Chem., 1996, 4 (7), 1077-1088]and imidazole (1.10 g, 16.1 mmol, 3.0 eq) in DMF (20 mL), at 0° C., was added tert-butyl(chloro)diphenylsilane (2.21 g, 8.05 mmol, 1.5 eq), in several portions over 10 min, and the reaction mixture was stirred at 25° C. for 16 h. The resulting mixture was partitioned between EtOAc and brine, the organic layer was dried over anhydrous Na₂SO₄ and the filtrate was concentrated under reduced pressure. The crude residue was purified by silica gel column chromatography (10:1 Petroleum ether: THF) to afford (3aR,6R,6aR)-6-{[(tert-butyldiphenylsilyl)oxy]methyl}-2,2-dimethyl-tetrahydrocyclopenta[d][1,3]dioxol-4-one 324c (1.30 g, 57%) as a white solid. LCMS [Cortecs C18+, 50*3.0 mm, 2.7 μm, Mobile Phase A: Water/5 mM NH₄HCO₃+0.05% Ammonia, Mobile Phase B: Acetonitrile, 5%-100% B-2 min (+−), 1.50 L/min, ES, m/z]: T_{R}=1.31 min; [M+H]⁺: 425.

### (3aS,4S,6R,6aR)-6-(((tert-Butyldiphenylsilyl)oxy)methyl)-2,2-dimethyltetrahydro-4H-cyclopenta[d][1,3]dioxol-4-ol (325c)

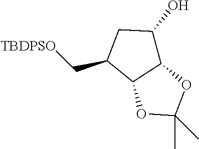

To a solution of (3aR,6R,6aR)-6-{[(tert-butyldiphenylsilyl)oxy]methyl}-2,2-dimethyl-tetrahydrocyclopenta[d][1,3]dioxol-4-one 324c (1.30 g, 3.10 mmol, 1.0 eq) in MeOH (20 mL), at 0° C., was added NaBH₄ (174 mg, 4.60 mmol, 1.5 eq), in several portions over 10 min, and the reaction mixture was stirred at 0° C. for 1 h. The resulting mixture was concentrated under reduced pressure and the crude residue was purified by silica gel column chromatography (4:1 Petroleum ether: THF) to afford (3aS,4S,6R,6aR)-6-{[(tert-butyldiphenylsilyl)oxy]methyl}-2,2-dimethyl-tetrahydro-3aH-cyclopenta[d][1,3]dioxol-4-ol 325c (1.10 g, 84%) as a white solid. LCMS [Cortecs C18+, 50*3.0 mm, 2.7 μm, Mobile Phase A: Water/5 mM NH₄HCO₃+0.05% Ammonia, Mobile Phase B: Acetonitrile/5% water: 5%-100% B-2.5 min (+−), 1.50 L/min, ES, m/z]: T_{R}=1.15 min; [M+H]⁺: 427.

### 2-chloro-5-(thiophen-2-yl)-7H-pyrrolo[2,3-d]pyrimidine (347c)

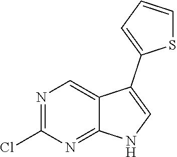 [SMILES: Clc1ncc2c(-c3cccs3)c[nH]c2n1]

To a solution of 5-bromo-2-chloro-7H-pyrrolo[2,3-d]pyrimidine (500 mg, 2.15 mmol, 1.0 eq) and thiophen-2-ylboronic acid (412 mg, 3.22 mmol, 1.5 eq) in dioxane (5.0 mL) and H₂O (0.5 mL) were added K₂CO₃ (891 mg, 6.45 mmol, 3.0 eq) and Pd (DtBPF) Cl₂ (280 mg, 0.43 mmol, 0.2 eq) and the reaction mixture was stirred at 100° C. for 1 h, under nitrogen. The reaction was quenched with water (10 mL) and then extracted with EtOAc (3×10 mL). The combined organic extracts were dried over anhydrous Na₂SO₄ and filtered. The filtrate was concentrated under reduced pressure and the crude residue was purified by silica gel column chromatography (1:1 Petroleum ether/AcOEt) to afford 2-chloro-5-(thiophen-2-yl)-7H-pyrrolo[2,3-d]pyrimidine 347c (300 mg, 59%) as a yellow solid. LCMS [column: L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min, 1.20 mL/min, ES, m/z]: T_{R}=1.75 min; [M+H]⁺: 236, 238.

### 7-((3aS,4R,6R,6aR)-6-(((tert-Butyldiphenylsilyl)oxy)methyl)-2,2-dimethyltetrahydro-4H-cyclopenta[d][1,3]dioxol-4-yl)-2-chloro-5-(thiophen-2-yl)-7H-pyrrolo[2,3-d]pyrimidine (326c)

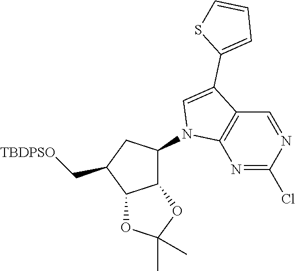

To a solution of (3aS,4S,6R,6aR)-6-{[(tert-butyldiphenylsilyl)oxy]methyl}-2,2-dimethyl-tetrahydro-3aH-cyclopenta[d][1,3]dioxol-4-ol 325c (0.40 g, 0.90 mmol, 1.0 eq), PPh₃ (0.49 g, 1.90 mmol, 2.0 eq) in toluene (20.0 mL) was added 2-chloro-5-(thiophen-2-yl)-7H-pyrrolo[2,3-d]pyrimidine 347c (0.22 g, 0.90 mmol, 1.0 eq), over 10 min, at 0° C. To the above mixture, at 0° C., was then added a solution of DBAD (0.43 g, 1.90 mmol, 2.1 eq) in toluene (1.0 mL), dropwise over 5 min, and the reaction mixture was stirred at 60° C. for 2 h. The resulting mixture was concentrated under reduced pressure and the crude residue was purified by silica gel column chromatography (5:1 Petroleum ether: THF) to afford 7-[(3aS,4R,6R,6aR)-6-{[(tert-butyldiphenylsilyl)oxy]methyl}-2,2-dimethyl-tetrahydro-3aH-cyclopenta[d][1,3]dioxol-4-yl]-2-chloro-5-(thiophen-2-yl)pyrrolo[2,3-d]pyrimidine 326c (400 mg, 66%) as a white solid. LCMS [Cortecs C18+, 50*3.0 mm, 2.7 μm, Mobile Phase A: Water/5 mM NH₄HCO₃+0.05% Ammonia, Mobile Phase B: Acetonitrile, 5%-100% B-3 min (+−), 1.50 L/min, ES, m/z]: T_{R}=1.71 min; [M+H]⁺: 644, 646.

### ((3aR,4R,6R,6aS)-6-(2-Chloro-5-(thiophen-2-yl)-7H-pyrrolo[2,3-d]pyrimidin-7-yl)-2,2-dimethyltetrahydro-4H-cyclopenta[d][1,3]dioxol-4-yl)methanol (327c)

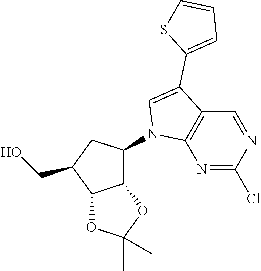 [SMILES: CC1(C)O[C@@H]2[C@@H](CO)C[C@@H](n3cc(-c4cccs4)c4cnc(Cl)nc43)[C@@H]2O1]

To a solution of 7-[(3aS,4R,6R,6aR)-6-{[(tert-butyldiphenylsilyl)oxy]methyl}-2,2-dimethyl-tetrahydro-3aH-cyclopenta[d][1,3]dioxol-4-yl]-2-chloro-5-(thiophen-2-yl)pyrrolo[2,3-d]pyrimidine 326c (400 mg, 0.62 mmol, 1.0 eq) in THF (10 mL), at 0° C., was added 1M TBAF (1M in THF, 0.74 mL, 0.74 mmol, 1.2 eq) dropwise over 10 min at 0° C., and the reaction mixture was stirred at 25° C. for 2 h. The resulting mixture was concentrated under reduced pressure and the crude residue was purified by silica gel column chromatography (15:1 DCM: MeOH) to afford [(3aR,4R,6R,6aS)-6-[2-chloro-5-(thiophen-2-yl)pyrrolo[2,3-d]pyrimidin-7-yl]-2,2-dimethyl-tetrahydro-3aH-cyclopenta[d][1,3]dioxol-4-yl]methanol 327c (280 mg, crude) as a colorless oil. LCMS [Cortecs C18+, 50*3.0 mm, 2.7 μm, Mobile Phase A: Water/5 mM NH₄HCO₃+0.05% Ammonia, Mobile Phase B: Acetonitrile, 5%-100% B-3 min (+−), 1.50 L/min, ES, m/z]: T_{R}=1.31 min; [M+H]⁺: 406, 408.

### (3aR,4S,6R,6aS)-6-(2-Chloro-5-(thiophen-2-yl)-7H-pyrrolo[2,3-d]pyrimidin-7-yl)-2,2-dimethyltetrahydro-4H-cyclopenta[d][1,3]dioxole-4-carbaldehyde (328c)

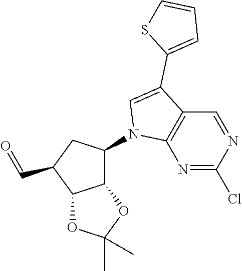 [SMILES: CC1(C)O[C@@H]2[C@H](O1)[C@@H](C=O)C[C@H]2n1cc(-c2cccs2)c2cnc(Cl)nc21]

To a solution of (3aR,4R,6R,6aS)-6-[2-chloro-5-(thiophen-2-yl)pyrrolo[2,3-d]pyrimidin-7-yl]-2,2-dimethyl-tetrahydro-3aH-cyclopenta[d][1,3]dioxol-4-yl]methanol 327c (150 mq, 0.37 mmol, 1.0 eq) and DCM (10 mL), at 0° C., was added DMP (172 mq, 0.41 mmol, 1.1 eq), in several portions over 10 min, and the reaction mixture was stirred at 25° C. for 2 h. The resulting mixture was filtered and the filter cake was washed with DCM (3×30 mL). The combined filtrate was concentrated under reduced pressure and the crude residue was purified by silica gel column chromatography (10:1 Petroleum ether: THF) to afford (3aR,4S,6R,6aS)-6-[2-chloro-5-(thiophen-2-yl)pyrrolo[2,3-d]pyrimidin-7-yl]-2,2-dimethyl-tetrahydro-3aH-cyclopenta[d][1,3]dioxole-4-carbaldehyde 328c (120 mg, 80%) as a colorless oil. LCMS [Cortecs C18+, 50*3.0 mm, 2.7 μm, Mobile Phase A: Water/5 mM NH₄HCO₃+0.05% Ammonia, Mobile Phase B: Acetonitrile, 5%-100% B-3 min (+−), 1.50 L/min, ES, m/z]: T_{R}=2.25 min; [M+H]⁺: 404, 406.

### (1R,2S,3R,5R)-3-(2-Chloro-5-(thiophen-2-yl)-7H-pyrrolo[2,3-d]pyrimidin-7-yl)-5-(((3-(phenethylamino)propyl)(1H-pyrazol-4-yl)amino)methyl)cyclopentane-1,2-diol (641)

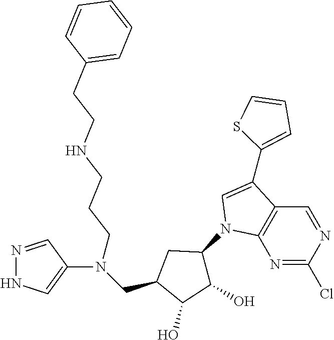 [SMILES: O[C@@H]1[C@@H](CN(CCCNCCc2ccccc2)c2cn[nH]c2)C[C@@H](n2cc(-c3cccs3)c3cnc(Cl)nc32)[C@@H]1O]

To a solution of (3aR,4S,6R,6aS)-6-[2-chloro-5-(thiophen-2-yl)pyrrolo[2,3-d]pyrimidin-7-yl]-2,2-dimethyl-tetrahydro-3aH-cyclopenta[d][1,3]dioxole-4-carbaldehyde 328c (120 mq, 0.29 mmol, 1.0 eq), tert-butyl-N-(2-phenylethyl)-N-[3-(1H-pyrazol-4-ylamino)propyl]carbamate 225c (120 mg, 0.35 mmol, 1.2 eq) and AcOH (36 mq, 0.58 mmol, 2 eq) in DCM (5.0 mL), at 0° C., was added NaBH (OAc) 3 (123 mg, 0.58 mmol, 2.0 eq) in several portions, over 2 min, and the reaction mixture was stirred at 25° C. for 2 h. The resulting mixture was concentrated under reduced pressure and 4M HCl in MeOH (2.0 mL, 8.00 mmol) was added, dropwise over 2 min, at 0° C. and the reaction mixture was stirred at 50° C. for 10 min. The resulting mixture was concentrated under reduced pressure and the crude residue was dissolved in MeOH (2.0 mL) and the solution was adjusted to pH 8 with NH₃·H₂O. The crude residue was purified by Prep-HPLC [Column: Welch Ultimate XB-C18, 50*250 mm, 10 μm; Mobile Phase A: 0.1% HCl, Mobile Phase B: ACN; Flow rate: 90 mL/min; Gradient: 5% B-5% B-3 min, 10% B-50% B-12mim]: to afford (1R,2S,3R,5R)-3-[2-chloro-5-(thiophen-2-yl)pyrrolo[2,3-d]pyrimidin-7-yl]-5-[{{3-[(2-phenylethyl)amino]propyl}(1H-pyrazol-4-yl)amino)methyl]cyclopentane-1,2-diol 641 (28.3 mg, 16%) as an off-white solid. LCMS [Cortecs C18+, 50*3.0 mm, 2.7 μm, Mobile Phase A: Water/0.02% TFA, Mobile Phase B: Acetonitrile, 5%-95% B-3 min, 1.50 L/min, ES, m/z]: T_{R}=1.05 min; [M+H]⁺: 592, 594. ¹H NMR (300 MHZ, DMSO-d₆) δ 12.22 (br s, 1H), 9.22 (s, 1H), 8.25 (s, 1H), 7.54 (d, J=4.4 Hz, 2H), 7.34-7.07 (m, 8H), 5.08-4.97 (m, 1H), 4.95 (d, J=6.5 Hz, 1H), 4.84-4.66 (m, 1H), 4.45-4.32 (m, 1H), 3.79 (d, J=5.3 Hz, 1H), 3.16 (dd, J=13.6, 6.8 Hz, 1H), 3.10-2.99 (m, 2H), 2.93 (dd, J=13.5, 7.0 Hz, 1H), 2.77-2.62 (m, 4H), 2.56 (d, J=6.8 Hz, 2H), 2.30-2.19 (m, 2H), 1.63-1.47 (m, 3H).

### Synthesis of (1R,2S,3R,5R)-3-[5-(1-benzylpyrazol-3-yl)-2-chloropyrrolo[2,3-d]pyrimidin-7-yl]-5-{[(3-{[2-(4-fluorophenyl)ethyl]amino}propyl)(methyl)amino]methyl}cyclopentane-1,2-diol (642)

### tert-Butyl-N-[3-({[(3aR,4R,6S,6aS)-6-hydroxy-2,2-dimethyl-tetrahydro-3aH-cyclopenta[d][1,3]dioxol-4-yl]methyl}amino)propyl]-N-[2-(4-fluorophenyl)ethyl]carbamate (329c)

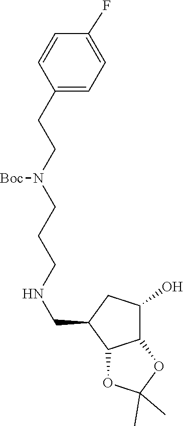

To a solution of (3aS,45,6R,6aR)-6-(aminomethyl)-2,2-dimethyl-tetrahydro-3aH-cyclopenta[d][1,3]dioxol-4-ol 225c (500 mg, 2.67 mmol, 1.0 eq), tert-butyl-N-[2-(4-fluorophenyl)ethyl]-N-(3-oxopropyl)carbamate 188c (394 mg, 1.33 mmol, 0.5 eq) and AcOH (160 mg, 2.67 mmol, 1.0 eq) in DCM (30 mL), at 0° C., was added NaBH (OAc) 3 (1.13 g, 5.34 mmol, 2.0 eq), in several portions over 5 min, and the reaction mixture was stirred at room temperature for 2 h. The resulting mixture was concentrated under reduced pressure and the crude residue was purified by silica gel column chromatography (15:1 DCM: MeOH) to afford tert-butyl-N-[3-({[(3aR,4R,6S,6aS)-6-hydroxy-2,2-dimethyl-tetrahydro-3aH-cyclopenta[d][1,3]dioxol-4-yl]methyl}amino)propyl]-N-[2-(4-fluorophenyl)ethyl]carbamate 329c (1.00 g, 80%) as a white solid. LCMS [Cortecs C18+, 50*3.0 mm, 2.7 μm, Mobile Phase A: Water/5 mM NH₄HCO₃+0.05% Ammonia Ammonia, Mobile Phase B: Acetonitrile/5% water: 5%-100% B-2.5 min (+−), 1.50 L/min, ES, m/z]: T_{R}=1.38 min; [M+H]⁺: 467.

### (1S,2R,3R,5R)-3-(Aminomethyl)-5-[5-(4-benzylthiophen-2-yl)pyrrolo[2,3-d]pyrimidin-7-yl]cyclopentane-1,2-diol (330c)

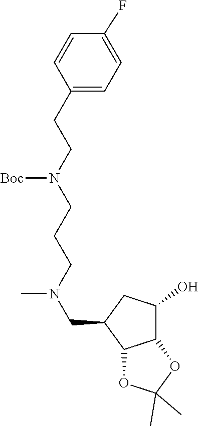

To a solution of tert-butyl-N-[3-({[(3aR,4R,6S,6aS)-6-hydroxy-2,2-dimethyl-tetrahydro-3aH-cyclopenta[d][1,3]dioxol-4-yl]methyl}amino)propyl]-N-[2-(4-fluorophenyl)ethyl]carbamate 329c (1.00 g. 2.14 mmol, 1.0 eq) and HCHO (0.32 g, 10.8 mmol, 5.0 eq) in DCM (30 mL), at 0° C., was added NaBH (OAc) 3 (0.91 g, 4.28 mmol, 2.0 eq), in several portions over 5 min, and the reaction mixture was stirred for 2 h at room temperature. The resulting mixture was concentrated under reduced pressure and the crude residue was purified by silica gel column chromatography (25:1 DCM: MeOH) to afford tert-butyl-N-[3-({[(3aR,4R,6S,6aS)-6-hydroxy-2,2-dimethyl-tetrahydro-3aH-cyclopenta[d][1,3]dioxol-4-yl]methyl}(methyl)amino)propyl]-N-[2-(4-fluorophenyl)ethyl]carbamate 330c (750 mg, 72%) as a white solid. LCMS [Cortecs C18+, 50*3.0 mm, 2.7 μm, Mobile Phase A: Water/5 mM NH₄HCO₃+0.05% Ammonia, Mobile Phase B: Acetonitrile/5% water: 5%-100% B-2.5 min (+−), 1.50 L/min, ES, m/z]: T_{R}=2.08 min; [M+H]⁺: 481.

### tert-Butyl-N-[3-({[(3aR,4R,6R,6aS)-6-{5-bromo-2-chloropyrrolo[2,3-d]pyrimidin-7-yl}-2,2-dimethyl-tetrahydro-3aH-cyclopenta[d][1,3]dioxol-4-yl]methyl}(methyl)amino)propyl]-N-[2-(4-fluorophenyl)ethyl]carbamate (331c)

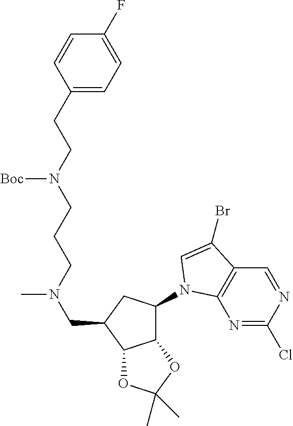 [SMILES: CN(CCCN(CCc1ccc(F)cc1)C(=O)OC(C)(C)C)C[C@H]1C[C@@H](n2cc(Br)c3cnc(Cl)nc32)[C@@H]2OC(C)(C)O[C@H]12]

To a solution of tert-butyl-N-[3-({[(3aR,4R,6S,6aS)-6-hydroxy-2,2-dimethyl-tetrahydro-3aH-cyclopenta[d][1,3]dioxol-4-yl]methyl}(methyl)amino)propyl]-N-[2-(4-fluorophenyl)ethyl]carbamate 330c (210 mg, 0.43 mmol, 1.0 eq), 5-bromo-2-chloro-7H-pyrrolo[2,3-d]pyrimidine (102 mg, 0.43 mmol, 1.0 eq) and PPh₃ (229 mg, 0.87 mmol, 2.0 eq) in toluene (20 mL) was added a solution of DBAD (201 mg, 0.87 mmol, 2.0 eq) in toluene (1.0 mL), dropwise over 5 min, at 0° C. and the reaction mixture was stirred at 60° C. for 2 h. The resulting mixture was concentrated under reduced pressure and the crude residue was purified by silica gel column chromatography (3:1 Petroleum ether: THF) to afford tert-butyl-N-[3-({[(3aR,4R,6R,6aS)-6-{5-bromo-2-chloropyrrolo[2,3-d]pyrimidin-7-yl}-2,2-dimethyl-tetrahydro-3aH-cyclopenta[d][1,3]dioxol-4-yl]methyl}(methyl)amino)propyl]-N-[2-(4-fluorophenyl)ethyl]carbamate 331c (160 mg, 52%) as a white solid. LCMS [Cortecs C18+, 50*3.0 mm, 2.7 μm, Mobile Phase A: Water/5 mM NH₄HCO₃+0.05% Ammonia, Mobile Phase B: Acetonitrile/5% water, 5%-100% B-2.5 min (+−), 1.50 L/min, ES, m/z]: T_{R}=2.13 min; [M+H]⁺: 694, 696.

### tert-Butyl-(3-((((3aR,4R,6R,6aS)-6-(5-(1-benzyl-1H-pyrazol-3-yl)-2-chloro-7H-pyrrolo[2,3-d]pyrimidin-7-yl)-2,2-dimethyltetrahydro-4H-cyclopenta[d][1,3]dioxol-4-yl)methyl)(methyl)amino)propyl)(4-fluorophenethyl)carbamate (332c)

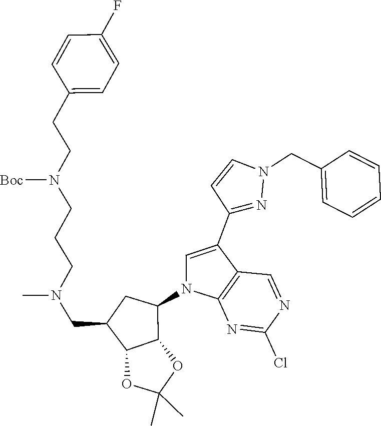 [SMILES: CN(CCCN(CCc1ccc(F)cc1)C(=O)OC(C)(C)C)C[C@H]1C[C@@H](n2cc(-c3ccn(Cc4ccccc4)n3)c3cnc(Cl)nc32)[C@@H]2OC(C)(C)O[C@H]12]

To a solution of tert-butyl-N-[3-({[(3aR,4R,6R,6aS)-6-{5-bromo-2-chloropyrrolo[2,3-d]pyrimidin-7-yl}-2,2-dimethyl-tetrahydro-3aH-cyclopenta[d][1,3]dioxol-4-yl]methyl}(methyl)amino)propyl]-N-[2-(4-fluorophenyl)ethyl]carbamate 331c (30 mg, 0.04 mmol, 1.0 eq) and 1-benzylpyrazol-3-ylboronic acid (17.4 mg, 0.08 mmol, 2.0 eq) [WO20170404450]in dioxane (2.0 mL) and H₂O (0.2 mL) were added K₂CO₃ (17.9 mg, 0.12 mmol, 3.0 eq) and Pd (DtBPF) Cl₂ (1.4 mg, 0.01 mmol, 0.05 eq) and the reaction mixture was stirred at 90° C. for 2 h, under nitrogen. The reaction was quenched with water (10 mL) and was then extracted with EtOAc (3×10 mL). The combined organic extracts were dried over anhydrous Na₂SO₄ and the filtrate was concentrated under reduced pressure. The crude residue was purified by silica gel column chromatography (1:1 Petroleum ether: THF) to afford tert-butyl-(3-((((3aR,4R,6R,6aS)-6-(5-(1-benzyl-1H-pyrazol-3-yl)-2-chloro-7H-pyrrolo[2,3-d]pyrimidin-7-yl)-2,2-dimethyltetrahydro-4H-cyclopenta[d][1,3]dioxol-4-yl)methyl)(methyl)amino)propyl)(4-fluorophenethyl)carbamate 332c (30 mg, 89%) as a white solid. LCMS [Cortecs C18+, 50*3.0 mm, 2.7 μm, Mobile Phase A: Water/5 mM NH₄HCO₃+0.05% Ammonia, Mobile Phase B: Acetonitrile/5% water: 5%-100% B-3.0 min (+−), 1.50 L/min, ES, m/z]: T_{R}=2.21 min; [M+H]⁺: 772, 774.

### (1R,2S,3R,5R)-3-[5-(1-Benzylpyrazol-3-yl)-2-chloropyrrolo[2,3-d]pyrimidin-7-yl]-5-{[(3-{[2-(4-fluorophenyl)ethyl]amino}propyl)(methyl)amino]methyl}cyclopentane-1,2-diol (642)

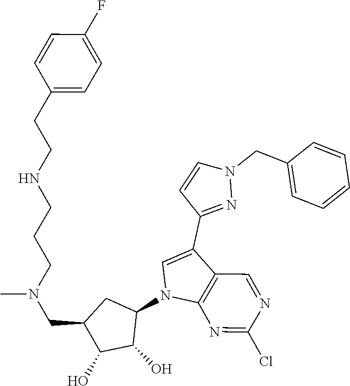 [SMILES: CN(CCCNCCc1ccc(F)cc1)C[C@H]1C[C@@H](n2cc(-c3ccn(Cc4ccccc4)n3)c3cnc(Cl)nc32)[C@H](O)[C@@H]1O]

To a solution of tert-butyl-N-[3-({[(3aR,4R,6R,6aS)-6-[5-(1-benzylpyrazol-3-yl)-2-chloropyrrolo[2,3-d]pyrimidin-7-yl]-2,2-dimethyl-tetrahydro-3aH-cyclopenta[d][1,3]dioxol-4-yl]methyl}(methyl)amino)propyl]-N-[2-(4-fluorophenyl)ethyl]carbamate 332c (30 mg, 0.04 mmol, 1.0 eq) in MeOH (2.0 mL) was added 4M HCl in MeOH (1.0 mL, 4.00 mmol) at 0° C. and the reaction mixture was stirred at 50° C. for 20 min. The resulting mixture was concentrated under reduced pressure, dissolved in MeOH (2.0 mL) and the solution was adjusted to pH 8 with NH₃·H₂O. The crude residue was purified by Prep-HPLC [Column: Welch Ultimate XB-C18, 50*250 mm, 10 μm; Mobile Phase A: 0.1% HCl, Mobile Phase B: ACN; Flow rate: 90 mL/min; Gradient: 5% B-5% B-3 min, 10% B-50% B-12mim]: to afford (1R,2S,3R,5R)-3-[5-(1-benzylpyrazol-3-yl)-2-chloropyrrolo[2,3-d]pyrimidin-7-yl]-5-{[(3-{[2-(4-fluorophenyl)ethyl]amino}propyl)(methyl)amino]methyl}cyclopentane-1,2-diol 642 (4.7 mg, 19%) as a white solid. LCMS [Cortecs C18+, 50*3.0 mm, 2.7 μm, Mobile Phase A: Water/0.02% TFA, Mobile Phase B: Acetonitrile, 5%-95% B-3 min, 1.50 L/min, ES, m/z]: T_{R}=0.93 min; [M+H]⁺: 632, 634. ¹H NMR (300 MHZ, DMSO-d₆+D₂O) δ 9.23 (s, 1H), 8.17 (s, 1H), 7.90 (s, 1H), 7.42-7.27 (m, 5H), 7.14-7.08 (m, 2H), 7.07-6.89 (m, 2H), 6.75 (s, 1H), 5.40 (s, 2H), 5.03-4.90 (m, 1H), 4.34-4.27 (m, 1H), 3.81-3.72 (m, 1H), 2.64-2.56 (m, 5H), 2.43-2.32 (m, 5H), 2.19-2.09 (m, 5H), 1.69-1.50 (m, 3H). Chiral-HPLC [Column: XA-RP YMC Cellulose-SB, 4.6*100 mm, 3 μm, 121AB00077; Mobile Phase A: Water (0.05% H₃PO₄), Mobile Phase B: acetonitrile; Total Flow: 1.00 mL/min; Conc. of Pump B: 10.0%; Oven Temperature: 25° C.]: T_{R}=3.42 min (10 min run), single peak.

### Synthesis of (1R,2S,3R,5R)-3-[4-amino-5-(1-benzylpyrazol-3-yl)-2-chloropyrrolo[2,3-d]pyrimidin-7-yl]-5-{[(3-{[2-(4-fluorophenyl)ethyl]amino}propyl)(methyl)amino]methyl}cyclopentane-1,2-diol (643)

### tert-Butyl-N-{[(3aR,4R,6R,6aS)-6-[4-amino-5-(1-benzylpyrazol-3-yl)-2-chloropyrrolo[2,3-d]pyrimidin-7-yl]-2,2-dimethyl-tetrahydro-3aH-cyclopenta[d][1,3]dioxol-4-yl]methyl}carbamate (333c)

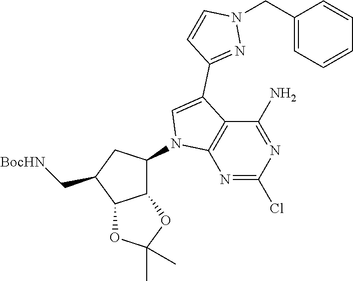 [SMILES: CC(C)(C)OC(=O)NC[C@H]1C[C@@H](n2cc(-c3ccn(Cc4ccccc4)n3)c3c(N)nc(Cl)nc32)[C@@H]2OC(C)(C)O[C@H]12]

To a solution of tert-butyl-N-{[(3aR,4R,6R,6aS)-6-{4-amino-5-bromo-2-chloropyrrolo[2,3-d]pyrimidin-7-yl}-2,2-dimethyl-tetrahydro-3aH-cyclopenta[d][1,3]dioxol-4-yl]methyl}carbamate (80 mg, 0.15 mmol, 1.0 eq) and (1-benzyl-1H-pyrazol-3-yl) boronic acid (44 mg, 0.15 mmol, 1.0 eq) [WO2017040445]]in dioxane (5.0 mL) and H₂O (0.5 mL) were added K₂CO₃ (64 mg, 0.46 mmol, 3.0 eq) and Pd (DtBPF) Cl₂ (5.1 mg, 0.01 mmol, 0.05 eq) and the reaction mixture was stirred at 80° C. for 2 h, under nitrogen. The reaction was quenched with water (10 mL) and then extracted with EtOAc (3×10 mL). The combined organic extracts were dried over anhydrous Na₂SO₄ and the filtrate was concentrated under reduced pressure. The crude residue was purified by silica gel column chromatography (15:1 DCM: MeOH) to afford tert-butyl-N-{[(3aR,4R,6R,6aS)-6-[4-amino-5-(1-benzylpyrazol-3-yl)-2-chloropyrrolo[2,3-d]pyrimidin-7-yl]-2,2-dimethyl-tetrahydro-3aH-cyclopenta[d][1,3]dioxol-4-yl]methyl}carbamate 333c (70 mg, 76%) as a pale brown solid. LCMS [Cortecs C18+, 50*3.0 mm, 2.7 μm, Mobile Phase A: Water/5 mM NH₄HCO₃+0.05% Ammonia, Mobile Phase B: Acetonitrile, 5%-100% B-2.0 min (+−), 1.50 L/min, ES, m/z): T_{R}=1.57 min; [M+H]⁺: 594 596.

### (1R,2S,3R,5R)-3-[4-Amino-5-(1-benzylpyrazol-3-yl)-2-chloropyrrolo[2,3-d]pyrimidin-7-yl]-5-(aminomethyl)cyclopentane-1,2-diol (334c)

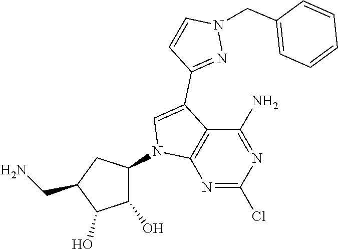 [SMILES: NC[C@H]1C[C@@H](n2cc(-c3ccn(Cc4ccccc4)n3)c3c(N)nc(Cl)nc32)[C@H](O)[C@@H]1O]

To tert-butyl-N-{[(3aR,4R,6R,6aS)-6-[4-amino-5-(1-benzylpyrazol-3-yl)-2-chloropyrrolo[2,3-d]pyrimidin-7-yl]-2,2-dimethyl-tetrahydro-3aH-cyclopenta[d][1,3]dioxol-4-yl]methyl}carbamate 333c (70 mg, 0.12 mmol, 1.0 eq) was added 4M HCl in MeOH (2.5 mL, 14.0 mmol) and the reaction mixture was stirred at 50° C. for 1 h. The resulting mixture was concentrated under reduced pressure to afford (1R,2S,3R,5R)-3-[4-amino-5-(1-benzylpyrazol-3-yl)-2-chloropyrrolo[2,3-d]pyrimidin-7-yl]-5-(aminomethyl)cyclopentane-1,2-diol 334c (53 mg, crude) as a white solid which was used in the next step without purification. LCMS [Cortecs C18+, 50*3.0 mm, 2.7 μm, Mobile Phase A: Water/5 mM NH₄HCO₃+0.05% Ammonia, Mobile Phase B: Acetonitrile, 5%-100% B-2.0 min (+−), 1.50 L/min, ES, m/z]: T_{R}=1.34 min; [M+H]⁺: 454, 456.

### tert-Butyl-N-[3-({[(1R,2R,3S,4R)-4-[4-amino-5-(1-benzylpyrazol-3-yl)-2-chloropyrrolo[2,3-d]pyrimidin-7-yl]-2,3-dihydroxycyclopentyl]methyl}amino)propyl]-N-[2-(4-fluorophenyl)ethyl]carbamate (335c)

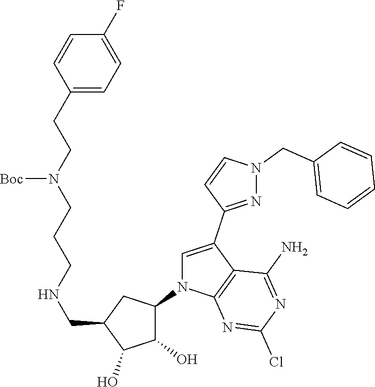 [SMILES: CC(C)(C)OC(=O)N(CCCNC[C@H]1C[C@@H](n2cc(-c3ccn(Cc4ccccc4)n3)c3c(N)nc(Cl)nc32)[C@H](O)[C@@H]1O)CCc1ccc(F)cc1]

To a solution of 1R,2S,3R,5R)-3-[4-amino-5-(1-benzylpyrazol-3-yl)-2-chloropyrrolo[2,3-d]pyrimidin-7-yl]-5-(aminomethyl)cyclopentane-1,2-diol (53 mg, 0.12 mmol, 1.0 eq) and tert-butyl-N-[2-(4-fluorophenyl)ethyl]-N-(3-oxopropyl)carbamate 334c (34.5 mg, 0.12 mmol, 1.0 eq) in DCM (2.0 mL) and MeOH (2.0 mL) was added DIEA (4.5 mg, 0.04 mmol, 0.3 eq) and the reaction mixture was heated at 40° C. for 30 min, under nitrogen. After this time, the resulting mixture was cooled to 0° C., NaBH₄ (8.8 mg, 0.23 mmol, 2.0 eq) was added and the reaction mixture was stirred at 0° C. for 0.5 h. The resulting mixture was concentrated under reduced pressure and the crude residue was purified by reverse flash chromatography (XBridge C18; mobile phase, MeCN in Ammonium bicarbonate water, 20% to 60% gradient in 10 min; detector, UV 254 nm) to afford tert-butyl-N-[3-({[(1R,2R,3S,4R)-4-[4-amino-5-(1-benzylpyrazol-3-yl)-2-chloropyrrolo[2,3-d]pyrimidin-7-yl]-2,3-dihydroxycyclopentyl]methyl}amino)propyl]-N-[2-(4-fluorophenyl)ethyl]carbamate 335c (61 mg, 71%) as a yellow solid. LCMS [Cortecs C18+, 50*3.0 mm, 2.7 μm, Mobile Phase A: Water/5 mM NH₄HCO₃+0.05% Ammonia, Mobile Phase B: Acetonitrile, 5%-100% B-2.5 min (+−), 1.50 L/min, ES, m/z]: T_{R}=1.73 min; [M+H]⁺: 734, 736.

### (1R,2S,3R,5R)-3-[4-Amino-5-(1-benzylpyrazol-3-yl)-2-chloropyrrolo[2,3-d]pyrimidin-7-yl]-5-{[(3-{[2-(4-fluorophenyl)ethyl]amino}propyl)(methyl)amino]methyl}cyclopentane-1,2-diol (643)

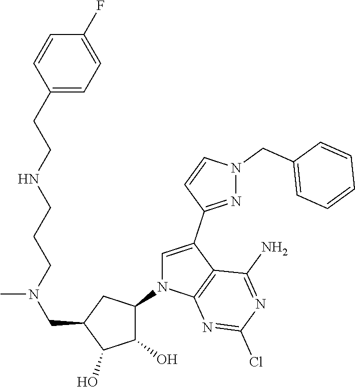 [SMILES: CN(CCCNCCc1ccc(F)cc1)C[C@H]1C[C@@H](n2cc(-c3ccn(Cc4ccccc4)n3)c3c(N)nc(Cl)nc32)[C@H](O)[C@@H]1O]

To a solution of tert-butyl-N-[3-({[(1R,2R,3S,4R)-4-[4-amino-5-(1-benzylpyrazol-3-yl)-2-chloropyrrolo[2,3-d]pyrimidin-7-yl]-2,3-dihydroxycyclopentyl]methyl}amino)propyl]-N-[2-(4-fluorophenyl)ethyl]carbamate (61 mg, 0.08 mmol, 1.0 eq), HCHO (5.0 mg, 0.16 mmol, 2.0 eq) and AcOH (0.3 mg, 0.01 mmol, 0.05 eq) in DCM (5.0 mL), at 0° C., was added NaBH (OAc) 3 (35.3 mg, 0.16 mmol, 2.0 eq), in several portions over 2 min, and the reaction mixture was stirred at 25° C. for 0.5 h. The resulting mixture was concentrated under reduced pressure and to the crude residue was added 4M HCl in MeOH (2.0 mL, 8.00 mmol), at 0° C., and the reaction mixture was stirred at 50° C. for 10 min. The resulting mixture was concentrated under reduced pressure, dissolved in MeOH (2.0 mL) and the solution was adjusted to pH 8 with NH₃·H₂O. The crude residue was purified by Prep-HPLC [Column: Welch Ultimate XB-C18, 50*250 mm, 10 μm; Mobile Phase A: 0.1% HCl, Mobile Phase B: ACN; Flow rate: 90 mL/min; 10% B-50% B-12mim]: to afford (1R,2S,3R,5R)-3-[4-amino-5-(1-benzylpyrazol-3-yl)-2-chloropyrrolo[2,3-d]pyrimidin-7-yl]-5-{[(3-{[2-(4-fluorophenyl)ethyl]amino}propyl)(methyl)amino]methyl}cyclopentane-1,2-diol 643 (7.1 mg, 13%) as an off-white solid. LCMS [Cortecs C18+, 50*3.0 mm, 2.7 μm, Mobile Phase A: Water/0.02% TFA, Mobile Phase B: Acetonitrile, 5%-95% B-3 min, 1.50 L/min, ES, m/z]: T_{R}=1.38 min; [M+H]⁺: 647, 649. ¹H NMR (300 MHZ, DMSO-d₆) δ 7.89 (d, J=2.3 Hz, 1H), 7.81 (s, 1H), 7.42-7.22 (m, 5H), 7.21-7.11 (m, 2H), 7.10-6.97 (m, 2H), 6.67 (d, J=2.3 Hz, 1H), 5.35 (s, 2H), 4.78-4.71 (m, 1H), 4.21 (dd, J=9.0, 5.4 Hz, 1H), 3.76-3.68 (m, 1H), 2.76-2.57 (m, 6H), 2.52-2.43 (m, 1H), 2.39-2.25 (m, 3H), 2.16-2.03 (m, 5H), 1.63-1.52 (m, 2H), 1.49-1.38 (m, 1H).

### Synthesis of (1R,2S,3R,5R)-3-[2-chloro-5-(1-methylpyrazol-3-yl)pyrrolo[2,3-d]pyrimidin-7-yl]-5-{[(3-{[2-(4-fluorophenyl)ethyl]amino}propyl)(methyl)amino]methyl}cyclopentane-1,2-diol (644)

### tert-Butyl-N-[3-({[(3aR,4R,6R,6aS)-6-[2-chloro-5-(1-methylpyrazol-3-yl)pyrrolo[2,3-d]pyrimidin-7-yl]-2,2-dimethyl-tetrahydro-3aH-cyclopenta[d][1,3]dioxol-4-yl]methyl}(methyl)amino)propyl]-N-[2-(4-fluorophenyl)ethyl]carbamate (336c) F

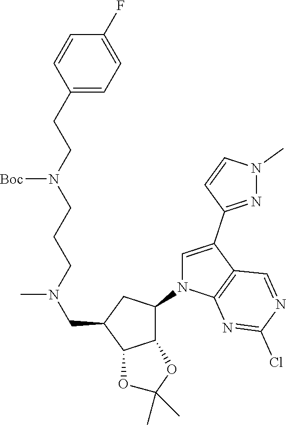

To a solution of tert-butyl-N-[3-({[(3aR,4R,6R,6aS)-6-{5-bromo-2-chloropyrrolo[2,3-d]pyrimidin-7-yl}-2,2-dimethyl-tetrahydro-3aH-cyclopenta[d][1,3]dioxol-4-yl]methyl}(methyl)amino)propyl]-N-[2-(4-fluorophenyl)ethyl]carbamate 331c (80 mg, 0.12 mmol, 1.0 eq) and 1-methyl-3-(4,4,5,5-tetramethyl-1,3,2-dioxaborolan-2-yl)pyrazole (47.9 mg, 0.23 mmol, 2.0 eq) in dioxane (2.0 mL) and H₂O (0.2 mL) were added K₂CO₃ (47.7 mg, 0.35 mmol, 3.0 eq) and Pd (DtBPF) Cl₂ (3.8 mg, 0.01 mmol, 0.05 eq) and the reaction mixture was stirred at 90° C. for 2 h, under nitrogen. The reaction was quenched with water (10 mL) and extracted with EtOAc (3×10 mL). The combined organic extracts were dried over anhydrous Na₂SO₄ and the filtrate was concentrated under reduced pressure. The crude residue was purified by silica gel column chromatography (1:1 Petroleum ether: THF) to afford tert-butyl-N-[3-({[(3aR,4R,6R,6aS)-6-[2-chloro-5-(1-methylpyrazol-3-yl)pyrrolo[2,3-d]pyrimidin-7-yl]-2,2-dimethyl-tetrahydro-3aH-cyclopenta[d][1,3]dioxol-4-yl]methyl}(methyl)amino)propyl]-N-[2-(4-fluorophenyl)ethyl]carbamate 336c (65 mg, 81%) as a white solid. LCMS [Cortecs C18+, 50*3.0 mm, 2.7 μm, Mobile Phase A: Water/5 mM NH₄HCO₃+0.05% Ammonia, Mobile Phase B: Acetonitrile/5% water: 5%-100% B-3.0 min (+−), 1.50 L/min, ES, m/z]: T_{R}=2.48 min; [M+H]⁺: 696, 698.

### (1R,2S,3R,5R)-3-[2-Chloro-5-(1-methylpyrazol-3-yl)pyrrolo[2,3-d]pyrimidin-7-yl]-5-{[(3-{[2-(4-fluorophenyl)ethyl]amino}propyl)(methyl)amino]methyl}cyclopentane-1,2-diol (644)

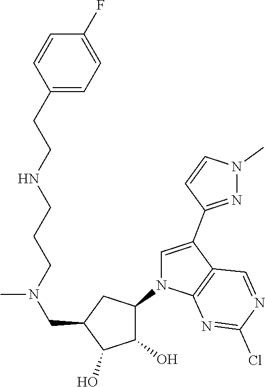

To tert-butyl-N-[3-({[(3aR,4R,6R,6aS)-6-[2-chloro-5-(1-methylpyrazol-3-yl)pyrrolo[2,3-d]pyrimidin-7-yl]-2,2-dimethyl-tetrahydro-3aH-cyclopenta[d][1,3]dioxol-4-yl]methyl}(methyl)amino)propyl]-N-[2-(4-fluorophenyl)ethyl]carbamate 336c (65 mg, 0.09 mmol, 1.0 eq) was added 4M HCl in MeOH (2.0 mL, 8.00 mmol) at 0° C. and the reaction mixture was stirred at 50° C. for 20 min. The resulting mixture was concentrated under reduced pressure, dissolved in MeOH (2.0 mL) and the solution was adjusted to pH 8 with NH₃·H₂O. The crude residue was purified by Prep-HPLC [Column: Welch Ultimate XB-C18, 50*250 mm, 10 μm; Mobile Phase A: 0.1% HCl, Mobile Phase B: ACN; Flow rate: 90 mL/min; Gradient: 5% B-5% B-3 min, 10% B-50% B-12mim)]: to afford (1R,2S,3R,5R)-3-[2-chloro-5-(1-methylpyrazol-3-yl)pyrrolo[2,3-d]pyrimidin-7-yl]-5-{[(3-{[2-(4-fluorophenyl)ethyl]amino}propyl)(methyl)amino]methyl}cyclopentane-1,2-diol 644 (10.3 mg, 19%) as a white solid. LCMS [Cortecs C18+, 50*3.0 mm, 2.7 μm, Mobile Phase A: Water/0.02% TFA, Mobile Phase B: Acetonitrile, 5%-95% B-3 min, 1.50 L/min, ES, m/z]: T_{R}=1.29 min; [M+H]⁺: 556, 558. ¹H NMR (300 MHZ, DMSO-d₆+D₂O) δ 9.26 (s, 1H), 8.04 (s, 1H), 7.76 (d, J=2.3 Hz, 1H), 7.27-7.16 (m, 2H), 7.13-7.01 (m, 2H), 6.67 (d, J=2.2 Hz, 1H), 4.59 (d, J=6.0 Hz, 1H), 4.45-4.32 (m, 1H), 4.14 (dd, J=13.9, 8.2 Hz, 1H), 3.91 (s, 3H), 3.73-3.67 (m, 1H), 3.59-3.52 (m, 1H), 2.75-2.63 (m, 4H), 2.61-2.55 (m, 1H), 2.45-2.18 (m, 4H), 2.09 (s, 3H), 1.66-1.56 (m, 1H), 1.54-1.43 (m, 2H), 1.12-1.01 (m, 1H). Chiral-HPLC [Column: XA-RP-YMC Cellulose-SC, 4.6*100 mm, 3 μm, 119IA70110; Mobile Phase A: Water (0.05% H₃PO₄), Mobile Phase B: Acetonorle; Total Flow: 1.00 mL/min; Conc. of Pump B: 10.0%; Oven Temperature: 25° C.]: T_{R}=2.70 min (10 min run), single peak.

### Synthesis of (1R,2S,3R,5R)-3-[4-amino-2-chloro-5-(1-methylpyrazol-3-yl)pyrrolo[2,3-d]pyrimidin-7-yl]-5-{[(3-{[2-(4-fluorophenyl)ethyl]amino}propyl)(methyl)amino]methyl}cyclopentane-1,2-diol (645)

### tert-Butyl-N-{[(3aR,4R,6R,6aS)-6-{5-bromo-2,4-dichloropyrrolo[2,3-d]pyrimidin-7-yl}-2,2-dimethyl-tetrahydro-3aH-cyclopenta[d][1,3]dioxol-4-yl]methyl}carbamate (337c)

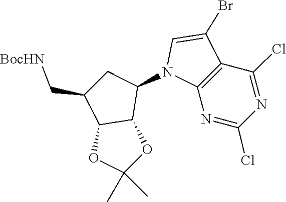 [SMILES: CC(C)(C)OC(=O)NC[C@H]1C[C@@H](n2cc(Br)c3c(Cl)nc(Cl)nc32)[C@@H]2OC(C)(C)O[C@H]12]

To a solution of tert-butyl-N-{[(3aR,4R,6S,6aS)-6-hydroxy-2,2-dimethyl-tetrahydro-3aH-cyclopenta[d][1,3]dioxol-4-yl]methyl}carbamate 242c (300 mg, 1.04 mmol, 1.0 eq) and PPh₃ (547 mg, 2.08 mmol, 2.0 eq) in toluene (30 mL) was added 5-bromo-2,4-dichloro-7H-pyrrolo[2,3-d]pyrimidine (223 mg, 0.83 mmol, 0.8 eq) and the resulting mixture was stirred at 0° C. for 10 min. To this mixture was then added a solution of DBAD (481 mg, 2.08 mmol, 2.0 eq) in toluene (1.0 mL), dropwise over 5 min, at 0° C. and the reaction mixture was stirred at 50° C. for 2 h. The resulting mixture was concentrated under reduced pressure and the crude residue was purified by silica gel column chromatography (10:1 Petroleum ether: THF) to afford tert-butyl-N-{[(3aR,4R,6R,6aS)-6-{5-bromo-2,4-dichloropyrrolo[2,3-d]pyrimidin-7-yl}-2,2-dimethyl-tetrahydro-3aH-cyclopenta[d][1,3]dioxol-4-yl]methyl}carbamate 337c (210 mg, 37%) as a white solid. LCMS [Cortecs C18+, 50*3.0 mm, 2.7 μm, Mobile Phase A: Water/5 mM NH₄HCO₃+0.05% Ammonia, Mobile Phase B: Acetonitrile, 5%-100% B-3.0 min (+−), 1.50 L/min, ES, m/z]: T_{R}=1.96 min; [M+H]⁺: 537, 539.

### tert-Butyl-N-{[(3aR,4R,6R,6aS)-6-{4-amino-5-bromo-2-chloropyrrolo[2,3-d]pyrimidin-7-yl}-2,2-dimethyl-tetrahydro-3aH-cyclopenta[d][1,3]dioxol-4-yl]methyl}carbamate (338c)

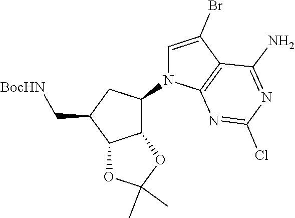 [SMILES: CC(C)(C)OC(=O)NC[C@H]1C[C@@H](n2cc(Br)c3c(N)nc(Cl)nc32)[C@@H]2OC(C)(C)O[C@H]12]

To tert-butyl-N-{[(3aR,4R,6R,6aS)-6-{5-bromo-2,4-dichloropyrrolo[2,3-d]pyrimidin-7-yl}-2,2-dimethyl-tetrahydro-3aH-cyclopenta[d][1,3]dioxol-4-yl]methyl}carbamate 337c (210 mg, 0.39 mmol, 1.0 eq) was added 7M NH₃ in MeOH (5.0 mL, 35 mmol) at room temperature and the reaction mixture was stirred at 80° C. for 2 h. The resulting mixture was concentrated under reduced pressure to afford tert-butyl-N-{[(3aR,4R,6R,6aS)-6-{4-amino-5-bromo-2-chloropyrrolo[2,3-d]pyrimidin-7-yl}-2,2-dimethyl-tetrahydro-3aH-cyclopenta[d][1,3]dioxol-4-yl]methyl}carbamate 338c (200 mg, crude) as a pale brown solid. LCMS [Cortecs C18+, 50*3.0 mm, 2.7 μm, Mobile Phase A: Water/5 mM NH₄HCO₃+0.05% Ammonia, Mobile Phase B: Acetonitrile, 5%-100% B-2.0 min (+−), 1.50 L/min, ES, m/z]: T_{R}=1.49 min; [M+H]⁺: 518, 520.

### tert-Butyl-N-{[(3aR,4R,6R,6aS)-6-[4-amino-2-chloro-5-(1-methylpyrazol-3-yl)pyrrolo[2,3-d]pyrimidin-7-yl]-2,2-dimethyl-tetrahydro-3aH-cyclopenta[d][1,3]dioxol-4-yl]methyl}carbamate (339c)

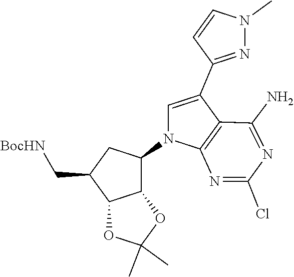 [SMILES: Cn1ccc(-c2cn([C@@H]3C[C@H](CNC(=O)OC(C)(C)C)[C@H]4OC(C)(C)O[C@H]43)c3nc(Cl)nc(N)c23)n1]

To a solution of tert-butyl-N-{[(3aR,4R,6R,6aS)-6-{4-amino-5-bromo-2-chloropyrrolo[2,3-d]pyrimidin-7-yl}-2,2-dimethyl-tetrahydro-3aH-cyclopenta[d][1,3]dioxol-4-yl]methyl}carbamate 338c (100 mg, 0.19 mmol, 1.0 eq) and 1-methylpyrazol-3-ylboronic acid (24.4 mg, 0.19 mmol, 1.0 eq) in dioxane (5.0 mL) and H₂O (0.5 mL) were added K₂CO₃ (80.2 mg, 0.58 mmol, 3.0 eq) and Pd (DtBPF) Cl₂ (6.3 mg, 0.01 mmol, 0.05 eq) and the reaction mixture was stirred at 80° C. for 2 h, under nitrogen. The reaction was quenched with water (10 mL) and then extracted with EtOAc (3×10 mL). The combined organic extracts were dried over anhydrous Na₂SO₄ and the filtrate was concentrated under reduced pressure. The crude residue was purified by silica gel column chromatography (10:1 DCM: MeOH) to afford tert-butyl-N-{[(3aR,4R,6R,6aS)-6-[4-amino-2-chloro-5-(1-methylpyrazol-3-yl)pyrrolo[2,3-d]pyrimidin-7-yl]-2,2-dimethyl-tetrahydro-3aH-cyclopenta[d][1,3]dioxol-4-yl]methyl}carbamate 339c (82 mg, 81%) as a pale brown solid. LCMS [Cortecs C18+, 50*3.0 mm, 2.7 μm, Mobile Phase A: Water/5 mM NH₄HCO₃+0.05% Ammonia, Mobile Phase B: Acetonitrile, 5%-100% B-2.0 min (+−), 1.50 L/min, ES, m/z]: T_{R}=1.95 min; [M+H]⁺: 518, 520.

### (1R,2S,3R,5R)-3-[4-Amino-2-chloro-5-(1-methylpyrazol-3-yl)pyrrolo[2,3-d]pyrimidin-7-yl]-5-(aminomethyl)cyclopentane-1,2-diol (340c)

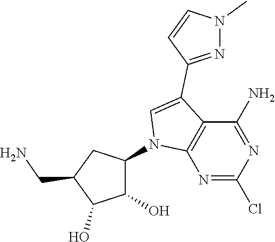 [SMILES: Cn1ccc(-c2cn([C@@H]3C[C@H](CN)[C@@H](O)[C@H]3O)c3nc(Cl)nc(N)c23)n1]

To tert-butyl-N-{[(3aR,4R,6R,6aS)-6-[4-amino-2-chloro-5-(1-methylpyrazol-3-yl)pyrrolo[2,3-d]pyrimidin-7-yl]-2,2-dimethyl-tetrahydro-3aH-cyclopenta[d][1,3]dioxol-4-yl]methyl}carbamate 339c (82 mg, 0.15 mmol, 1.0 eq) was added 4M HCl in MeOH (2.5 mL, 10 mmol) and the reaction mixture was stirred at 50° C. for 1 h. The resulting mixture was concentrated under reduced pressure to afford (1R,2S,3R,5R)-3-[4-amino-2-chloro-5-(1-methylpyrazol-3-yl)pyrrolo[2,3-d]pyrimidin-7-yl]-5-(aminomethyl)cyclopentane-1,2-diol 340c (58 mg, crude) as a white solid. LCMS [Cortecs C18+, 50*3.0 mm, 2.7 μm, Mobile Phase A: Water/5 mM NH₄HCO₃+0.05% Ammonia, Mobile Phase B: Acetonitrile, 5%-100% B-2.0 min (+−), 1.50 L/min, ES, m/z]: T_{R}=0.87 min; [M+H]⁺: 378, 580.

### tert-Butyl-N-[3-({[(3aR,4R,6R,6aS)-6-{5-bromo-2-chloropyrrolo[2,3-d]pyrimidin-7-yl}-2,2-dimethyl-tetrahydro-3aH-cyclopenta[d][1,3]dioxol-4-yl]methyl}(methyl)amino)propyl]-N-[2-(4-fluorophenyl)ethyl]carbamate (341c)

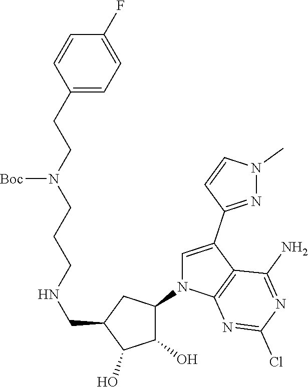 [SMILES: Cn1ccc(-c2cn([C@@H]3C[C@H](CNCCCN(CCc4ccc(F)cc4)C(=O)OC(C)(C)C)[C@@H](O)[C@H]3O)c3nc(Cl)nc(N)c23)n1]

To a solution of (1R,2S,3R,5R)-3-[4-amino-2-chloro-5-(1-methylpyrazol-3-yl)pyrrolo[2,3-d]pyrimidin-7-yl]-5-(aminomethyl)cyclopentane-1,2-diol 340c (58 mg, 0.15 mmol, 1.0 eq) and tert-butyl-N-[2-(4-fluorophenyl)ethyl]-N-(3-oxopropyl)carbamate 188c (45.3 mg, 0.15 mmol, 1.0 eq) in DCM (2.0 mL) and MeOH (2.0 mL) was added with DIEA (6.0 mg, 0.04 mmol, 0.3 eq) and the resulting mixture was stirred at 40° C. for 30 min, under nitrogen. To this mixture was then added NaBH₄ (11.6 mg, 0.31 mmol, 2.0 eq), in several portions at 0° C., and the reaction mixture was stirred at 0° C. for 0.5 h. The resulting mixture was concentrated under reduced pressure and the crude residue was purified by reverse flash chromatography (XBridge C18; mobile phase, MeCN in Ammonium bicarbonate water, 20% to 60% gradient in 10 min; detector, UV 254 nm): to afford tert-butyl-N-[3-({[(1R,2R,3S,4R)-4-[4-amino-2-chloro-5-(1-methylpyrazol-3-yl)pyrrolo[2,3-d]pyrimidin-7-yl]-2,3-dihydroxycyclopentyl]methyl}amino)propyl]-N-[2-(4-fluorophenyl)ethyl]carbamate 341c (50 mg, 49%) as a yellow solid. LCMS [Cortecs C18+, 50*3.0 mm, 2.7 μm, Mobile Phase A: Water/5 mM NH₄HCO₃+0.05% Ammonia, Mobile Phase B: Acetonitrile, 5%-100% B-2.5 min (+−), 1.50 L/min, ES, m/z]: T_{R}=1.50 min; [M+H]⁺: 658, 660.

### (1R,2S,3R,5R)-3-[4-Amino-2-chloro-5-(1-methylpyrazol-3-yl)pyrrolo[2,3-d]pyrimidin-7-yl]-5-{[(3-{[2-(4-fluorophenyl)ethyl]amino}propyl)(methyl)amino]methyl}cyclopentane-1,2-diol (645)

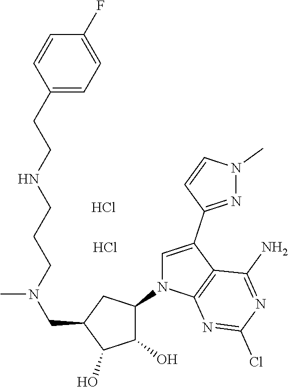 [SMILES: CN(CCCNCCc1ccc(F)cc1)C[C@H]1C[C@@H](n2cc(-c3ccn(C)n3)c3c(N)nc(Cl)nc32)[C@H](O)[C@@H]1O ; Cl ; Cl]

To a solution of tert-butyl-N-[3-({[(1R,2R,3S,4R)-4-[4-amino-2-chloro-5-(1-methylpyrazol-3-yl)pyrrolo[2,3-d]pyrimidin-7-yl]-2,3-dihydroxycyclopentyl]methyl}amino)propyl]-N-[2-(4-fluorophenyl)ethyl]carbamate 341c (58 mg, 0.08 mmol, 1.0 eq), HCHO (5.0 mg, 0.16 mmol, 2.0 eq) and AcOH (0.31 mg, 0.005 mmol, 0.05 eq) in DCM (5.0 mL), at 0° C., was added NaBH (OAc) 3 (28.1 mg, 0.13 mmol, 1.5 eq), in several portions over 2 min, and the reaction mixture was stirred at 25° C. for 2 h. The resulting mixture was concentrated under reduced pressure, 4M HCl in MeOH (2.0 mL, 8.00 mmol) was added and the reaction mixture was stirred at 50° C. for 10 min. The resulting mixture was concentrated under reduced pressure, the residue was dissolved in MeOH (2.0 mL) and the solution was adjusted to pH 8 with NH₃·H₂O. The crude residue was purified by Prep-HPLC [Column: Welch Ultimate XB-C18, 50*250 mm, 10 μm; Mobile Phase A: 0.1% HCl, Mobile Phase B: ACN; Flow rate: 90 mL/min; Gradient: 5% B-5% B-3 min, 10% B-50% B-12mim]: to afford (1R,2S,3R,5R)-3-[4-amino-2-chloro-5-(1-methylpyrazol-3-yl)pyrrolo[2,3-d]pyrimidin-7-yl]-5-{[(3-{[2-(4-fluorophenyl)ethyl]amino}propyl)(methyl)amino]methyl}cyclopentane-1,2-diol bis-HCl salt. 645 (8.5 mg, 16%) as an off-white solid. LCMS [Cortecs C18+, 50*3.0 mm, 2.7 μm, Mobile Phase A: Water/0.02% TFA, Mobile Phase B: Acetonitrile, 5%-95% B-3 min, 1.50 L/min, ES, m/z]: T_{R}=1.15 min; [M+H]⁺: 572, 574. ¹H NMR (300 MHZ, DMSO-d₆+D₂O) δ 7.79 (s, 1H), 7.54 (s, 1H), 7.33 (s, 1H), 7.18-7.12 (m, 2H), 7.07-6.99 (m, 2H), 4.89-4.78 (m, 1H), 4.21 (dd, J=8.9, 5.4 Hz, 1H), 3.88 (s, 3H), 3.69 (dd, J=5.4, 2.7 Hz, 1H), 2.69-2.58 (m, 4H), 2.44-2.25 (m, 4H), 2.20-2.07 (m, 5H), 1.62-1.40 (m, 3H)

### Synthesis of (1R,2S,3R,5R)-3-(4-amino-5-(4-benzylthiazol-2-yl)-2-chloro-7H-pyrrolo[2,3-d]pyrimidin-7-yl)-5-(((3-((4-fluorophenethyl)amino)propyl)amino)methyl)cyclopentane-1,2-diol (646)

### tert-Butyl-(((3aR,4R,6R,6aS)-6-(2,4-dichloro-5-iodo-7H-pyrrolo[2,3-d]pyrimidin-7-yl)-2,2-dimethyltetrahydro-4H-cyclopenta[d][1,3]dioxol-4-yl)methyl)carbamate (348c)

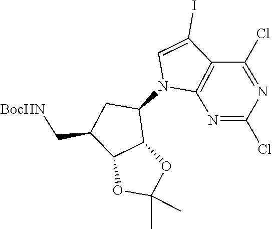 [SMILES: CC(C)(C)OC(=O)NC[C@H]1C[C@@H](n2cc(I)c3c(Cl)nc(Cl)nc32)[C@@H]2OC(C)(C)O[C@H]12]

To a stirred solution of tert-butyl-N-{[(3aR,4R,6S,6aS)-6-hydroxy-2,2-dimethyl-tetrahydro-3aH-cyclopenta[d][1,3]dioxol-4-yl]methyl}carbamate 242c (0.30 g, 1.00 mmol, 1.2 eq), 2,4-dichloro-5-iodo-7H-pyrrolo[2,3-d]pyrimidine (0.27 g, 0.90 mmol, 1.0 eq) and PPh₃ (0.45 g, 1.7 mmol, 2.0 eq) in toluene (3.0 mL), was added, dropwise at room temperature under N₂, a solution of DBAD (0.40 g, 1.70 mmol, 2.0 eq) in toluene (3.0 mL) and the reaction mixture was stirred at 60° C. for 3 h. The resulting mixture was allowed to cool to room temperature and was concentrated under reduced pressure. The crude residue was dissolved in DCM (3.0 mL) and purified by silica gel column chromatography (70:30 Petroleum ether/THF) to afford tert-butyl-(((3aR,4R,6R,6aS)-6-(2,4-dichloro-5-iodo-7H-pyrrolo[2,3-d]pyrimidin-7-yl)-2,2-dimethyltetrahydro-4H-cyclopenta[d][1,3]dioxol-4-yl)methyl)carbamate 348c (120 mg, 23%) as a pale yellow solid. LCMS [conditions Proshell HPH-C18, 50*3.0 mm, 2.7 μm, Mobile Phase A: Water (0.05% ammonia water), Mobile Phase B: Acetonitrile, 5%-100% B-2.5 min-1.2 mL-P (+−), 1.50 L/min, ES, m/z]: T_{R}=1.90 min; [M+H]⁺: 583, 585.

### tert-Butyl-(((3aR,4R,6R,6aS)-6-(4-amino-2-chloro-5-iodo-7H-pyrrolo[2,3-d]pyrimidin-7-yl)-2,2-dimethyltetrahydro-4H-cyclopenta[d][1,3]dioxol-4-yl)methyl)carbamate (349c)

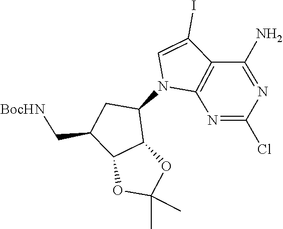 [SMILES: CC(C)(C)OC(=O)NC[C@H]1C[C@@H](n2cc(I)c3c(N)nc(Cl)nc32)[C@@H]2OC(C)(C)O[C@H]12]

To (((3aR,4R,6R,6aS)-6-(2,4-dichloro-5-iodo-7H-pyrrolo[2,3-d]pyrimidin-7-yl)-2,2-dimethyltetrahydro-4H-cyclopenta[d][1,3]dioxol-4-yl)methyl)carbamate 348c (0.10 g, 0.17 mmol, 1.0 eq) was added 7M NH₃ in MeOH (4.0 mL, 28 mmol) at room temperature and the reaction mixture was stirred at 80° C. for 2 h. The resulting mixture was allowed to cool to room temperature and was concentrated under reduced pressure to afford tert-butyl-(((3aR,4R,6R,6aS)-6-(4-amino-2-chloro-5-iodo-7H-pyrrolo[2,3-d]pyrimidin-7-yl)-2,2-dimethyltetrahydro-4H-cyclopenta[d][1,3]dioxol-4-yl)methyl)carbamate 349c (90 mg, crude) which was used in the next step directly without purification. LCMS [conditions Proshell HPH-C18, 50*3.0 mm, 2.7 μm, Mobile Phase A: Water (0.05% ammonia water), Mobile Phase B: Acetonitrile, 5%-100% B-2.5 min-1.2 mL-P (+−), 1.50 L/min, ES, m/z]: T_{R}=1.66 min; [M+H]⁺: 564, 566.

### tert-Butyl-(((3aR,4R,6R,6aS)-6-(4-amino-5-(4-benzylthiazol-2-yl)-2-chloro-7H-pyrrolo[2,3-d]pyrimidin-7-yl)-2,2-dimethyltetrahydro-4H-cyclopenta[d][1,3]dioxol-4-yl)methyl)carbamate (350c)

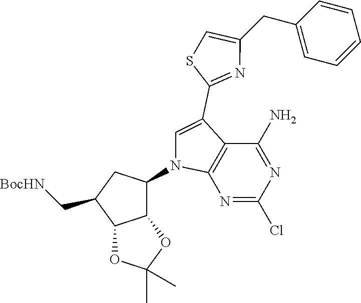 [SMILES: CC(C)(C)OC(=O)NC[C@H]1C[C@@H](n2cc(-c3nc(Cc4ccccc4)cs3)c3c(N)nc(Cl)nc32)[C@@H]2OC(C)(C)O[C@H]12]

To a stirred solution of tert-butyl-(((3aR,4R,6R,6aS)-6-(4-amino-2-chloro-5-iodo-7H-pyrrolo[2,3-d]pyrimidin-7-yl)-2,2-dimethyltetrahydro-4H-cyclopenta[d][1,3]dioxol-4-yl)methyl)carbamate 349c (90 mg, 0.16 mmol, 1.0 eq), Pd (PPh₃) 2Cl₂ (22.4 mg, 0.032 mmol, 0.2 eq) and K₃PO₄ (0.10 g, 0.50 mmol, 3.0 eq) in toluene (2.0 mL) was added, dropwise at room temperature, a solution of (4-benzyl-1,3-thiazol-2-yl)(chloro) zinc 227c (0.40 mmol crude in 5.0 mL THF, 2.5 eq) and the reaction mixture was stirred at 60° C. for 4 h. The resulting mixture was allowed to cool to room temperature, was quenched by the addition of saturated NH₄Cl solution and the resulting mixture was extracted with AcOEt (3×10 mL). The combined organic layers were washed with brine, dried over anhydrous Na₂SO₄ and the filtrate was concentrated under reduced pressure. The crude residue was purified by silica gel column chromatography (50:50 Petroleum ether/THF) to afford tert-butyl-(((3aR,4R,6R,6aS)-6-(4-amino-5-(4-benzylthiazol-2-yl)-2-chloro-7H-pyrrolo[2,3-d]pyrimidin-7-yl)-2,2-dimethyltetrahydro-4H-cyclopenta[d][1,3]dioxol-4-yl)methyl)carbamate 350c (50 mg, 51%) as colourless oil. LCMS [conditions Proshell HPH-C18, 50*3.0 mm, 2.7 μm, Mobile Phase A: Water/0.05% TFA, Mobile Phase B: Acetonitrile, 2%-95% B-5 min (+).lcm, 1.50 L/min, ES, m/z]: T_{R}=3.04 min; [M+H]⁺: 611, 613.

### (1R,2S,3R,5R)-3-(4-Amino-5-(4-benzylthiazol-2-yl)-2-chloro-7H-pyrrolo[2,3-d]pyrimidin-7-yl)-5-(aminomethyl)cyclopentane-1,2-diol (351c)

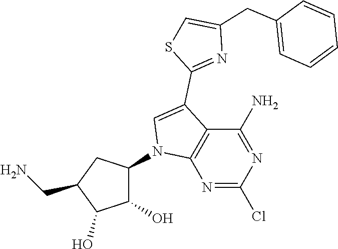 [SMILES: NC[C@H]1C[C@@H](n2cc(-c3nc(Cc4ccccc4)cs3)c3c(N)nc(Cl)nc32)[C@H](O)[C@@H]1O]

To tert-butyl-(((3aR,4R,6R,6aS)-6-(4-amino-5-(4-benzylthiazol-2-yl)-2-chloro-7H-pyrrolo[2,3-d]pyrimidin-7-yl)-2,2-dimethyltetrahydro-4H-cyclopenta[d][1,3]dioxol-4-yl)methyl)carbamate 350c (45 mg, 0.074 mmol, 1.0 eq), at room temperature, was added 4M HCl in MeOH (2.0 mL, 8.00 mmol) and the reaction mixture was stirred at 60° C. for 30 mim. The resulting mixture was concentrated under reduced pressure to afford (1R,2S,3R,5R)-3-[4-amino-5-(4-benzyl-1,3-thiazol-2-yl)-2-chloropyrrolo[2,3-d]pyrimidin-7-yl]-5-(aminomethyl)cyclopentane-1,2-diol HCl salt 351c (30 mg, crude) as a white solid was used in the next step directly without purification. LCMS [conditions Proshell HPH-C18, 50*3.0 mm, 2.7 μm, Mobile Phase A: Water/0.05% TFA, Mobile Phase B: Acetonitrile, 2%-100% B-2 min (+).lcm, 1.50 L/min, ES, m/z]: T_{R}=0.77 min; [M+H]⁺: 471, 473.

### tert-Butyl-(3-((((1R,2R,3S,4R)-4-(4-amino-5-(4-benzylthiazol-2-yl)-2-chloro-7H-pyrrolo[2,3-d]pyrimidin-7-yl)-2,3-dihydroxycyclopentyl)methyl)amino)propyl)(4-fluorophenethyl)carbamate (352c)

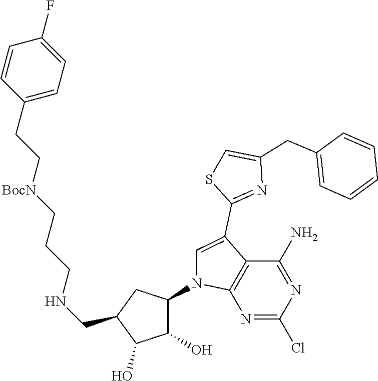 [SMILES: CC(C)(C)OC(=O)N(CCCNC[C@H]1C[C@@H](n2cc(-c3nc(Cc4ccccc4)cs3)c3c(N)nc(Cl)nc32)[C@H](O)[C@@H]1O)CCc1ccc(F)cc1]

To (1R,2S,3R,5R)-3-[4-amino-5-(4-benzyl-1,3-thiazol-2-yl)-2-chloropyrrolo[2,3-d]pyrimidin-7-yl]-5-(aminomethyl)cyclopentane-1,2-diol HCl salt 351c (25 mg, 0.05 mmol, 1.0 eq) and tert-butyl-N-[2-(4-fluorophenyl)ethyl]-N-(3-oxopropyl)carbamate 188c (10 mg, 0.035 mmol, 0.7 eq) in DCM (2.0 mL) was added DIEA (20 mg, 0.15 mmol, 3 eq) and the resulting mixture was stirred at room temperature for 1 h. MeOH (0.5 mL) and then NaBH₄ (6 mg, 0.15 mmol, 3.0 eq) were added and the reaction mixture was stirred at 0° C. for 0.5 h. The resulting mixture was concentrated under reduced pressure. The crude residue was dissolved in DCM (2.0 mL) and then purified by Prep-TLC (50:50 Petroleum ether/THF) to afford tert-butyl-(3-((((1R,2R,3S,4R)-4-(4-amino-5-(4-benzylthiazol-2-yl)-2-chloro-7H-pyrrolo[2,3-d]pyrimidin-7-yl)-2,3-dihydroxycyclopentyl)methyl)amino)propyl)(4-fluorophenethyl)carbamate 352c (20 mg, 50%) as a colourless oil. LCMS [conditions L-column3 ODS, 50*3.0 mm, 3.0 μm, Mobile Phase A: Water/5 mM NH₄HCO₃+0.05% Ammonia, Mobile Phase B: Acetonitrile/5% water, 2%-100% B-2.5 min (+), 1.50 L/min, ES, m/z]: T_{R}=1.82 min; [M+H]⁺: 750, 752.

### (1R,2S,3R,5R)-3-(4-Amino-5-(4-benzylthiazol-2-yl)-2-chloro-7H-pyrrolo[2,3-d]pyrimidin-7-yl)-5-(((3-((4-fluorophenethyl)amino)propyl)amino)methyl)cyclopentane-1,2-diol (646)

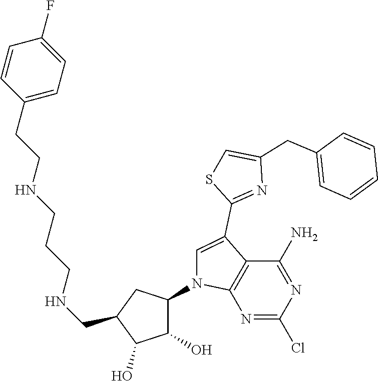 [SMILES: Nc1nc(Cl)nc2c1c(-c1nc(Cc3ccccc3)cs1)cn2[C@@H]1C[C@H](CNCCCNCCc2ccc(F)cc2)[C@@H](O)[C@H]1O]

To tert-butyl-(3-((((1R,2R,3S,4R)-4-(4-amino-5-(4-benzylthiazol-2-yl)-2-chloro-7H-pyrrolo[2,3-d]pyrimidin-7-yl)-2,3-dihydroxycyclopentyl)methyl)amino)propyl)(4-fluorophenethyl)carbamate 352c (20 mg, 0.027 mmol, 1.0 eq) was added 4M HCl in MeOH (1.5 mL, 6.0 mmol) at room temperature and the reaction mixture was stirred at 40° C. for 20 min. The resulting mixture was concentrated under reduced pressure. The crude residue was dissolved in CH₃CN (2.0 mL) and adjusted to pH 8 with NH₃·H₂O and the resulting mixture was purified by Prep-HPLC [Column: YMC-Actus Triart C18, 30*150 mm, 5 μm; Mobile Phase A: Water (10 mmol/L NH₄HCO₃+0.1% NH₃·H₂O), Mobile Phase B: ACN; Flow rate: 30 ml/min; Gradient: 24% B to 49% B in 10 min, 49% B; Wave Length: 220 nm]: to afford (1R,2S,3R,5R)-3-(4-amino-5-(4-benzylthiazol-2-yl)-2-chloro-7H-pyrrolo[2,3-d]pyrimidin-7-yl)-5-(((3-((4-fluorophenethyl)amino)propyl)amino)methyl)cyclopentane-1,2-diol 646 (9.3 mg, 51%) as a white solid. LCMS [ZORBAX SB-Aq, 50*4.6 mm, 1.8 μm, Mobile Phase A: Water/0.02% TFA, Mobile Phase B: Acetonitrile, 5%-95% B-3 min, 1.50 L/min, ES, m/z]: T_{R}=1.69 min; [M+H]⁺: 650, 652. 1H NMR (300 MHZ, DMSO-d₆) δ9.44 (s, 1H), 8.16 (s, 1H), 7.87 (s, 1H), 7.42-7.17 (m, 8H), 7.10-6.91 (m, 2H), 4.98-4.71 (m, 2H), 4.31-4.21 (m, 1H), 4.07 (s, 2H), 3.82-3.71 (m, 1H), 2.87-2.51 (m, 10H), 2.20-2.11 (m, 1H), 2.09-2.01 (m, 1H), 1.63-1.42 (m, 3H).

### Synthesis of (1R,2S,3R,5R)-3-(4-amino-5-(4-benzylthiazol-2-yl)-2-chloro-7H-pyrrolo[2,3-d]pyrimidin-7-yl)-5-(((3-((4-fluorophenethyl)amino)propyl)(methyl)amino)methyl)cyclopentane-1,2-diol (647)

### tert-Butyl-(3-((((1R,2R,35,4R)-4-(4-amino-5-(4-benzylthiazol-2-yl)-2-chloro-7H-pyrrolo[2,3-d]pyrimidin-7-yl)-2,3-dihydroxycyclopentyl)methyl)(methyl)amino)propyl)(4-fluorophenethyl)carbamate (353c)

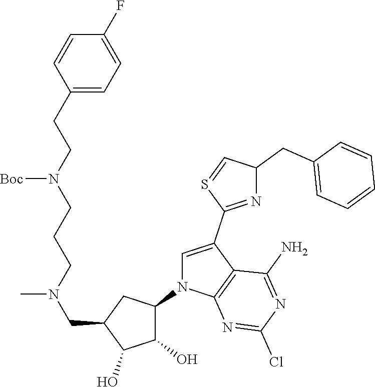

To a solution of tert-butyl-N-[3-({[(1R,2R,3S,4R)-4-[4-amino-5-(4-benzyl-1,3-thiazol-2-yl)-2-chloropyrrolo[2,3-d]pyrimidin-7-yl]-2,3-dihydroxycyclopentyl]methyl}amino)propyl]-N-[2-(4-fluorophenyl)ethyl]carbamate 352c (15 mg, 0.02 mmol, 1.0 eq) in THF (0.5 mL) and MeOH (0.5 mL) were added 30% HCHO (3.0 mg, 0.03 mmol, 1.5 eq), NaBH₃CN (2.5 mg, 0.04 mmol, 2.0 eq) and DIEA (5.2 mg, 0.04 mmol, 2.0 eq) and the reaction mixture was stirred at room temperature for 30 min. The resulting mixture was concentrated under reduced pressure and the crude residue was purified by prep-HPLC [Column: Welch Ultimate XB-C18, 50*250 mm, 10 μm; Mobile Phase A: Water/5 mM NH₄HCO₃, Mobile Phase B: ACN; Flow rate: 90 mL/min; Gradient: 5% B-5% B-2 min, 10% B-60% B-12 mim]: to afford tert-butyl-N-[3-({[(1R,2R,3S,4R)-4-[4-amino-5-(4-benzyl-1,3-thiazol-2-yl)-2-chloropyrrolo[2,3-d]pyrimidin-7-yl]-2,3-dihydroxycyclopentyl]methyl}(methyl)amino)propyl]-N-[2-(4-fluorophenyl)ethyl]carbamate 353c (13 mg, 85%) as an off-white solid. LCMS [conditions L-column3 ODS, 50*3.0 mm, 3.0 μm, Mobile PhaseA: Water/5 mM NH₄HCO₃+0.05% Ammonia, Mobile Phase B: Acetonitrile/5% water, 2%-100% B-2 min (+), 1.50 L/min, ES, m/z]: T_{R}=1.61 min; [M+H]⁺: 764, 766.

### (1R,2S,3R,5R)-3-(4-Amino-5-(4-benzylthiazol-2-yl)-2-chloro-7H-pyrrolo[2,3-d]pyrimidin-7-yl)-5-(((3-((4-fluorophenethyl)amino)propyl)(methyl)amino)methyl)cyclopentane-1,2-diol (647)

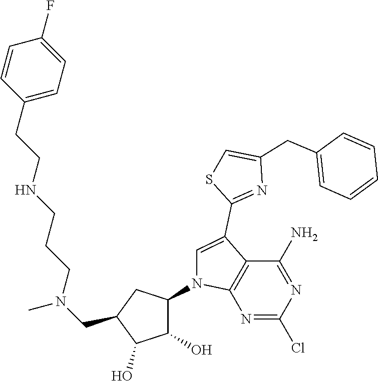 [SMILES: CN(CCCNCCc1ccc(F)cc1)C[C@H]1C[C@@H](n2cc(-c3nc(Cc4ccccc4)cs3)c3c(N)nc(Cl)nc32)[C@H](O)[C@@H]1O]

To tert-butyl-N-[3-({[(1R,2R,3S,4R)-4-[4-amino-5-(4-benzyl-1,3-thiazol-2-yl)-2-chloropyrrolo[2,3-d]pyrimidin-7-yl]-2,3-dihydroxycyclopentyl]methyl}(methyl)amino)propyl]-N-[2-(4-fluorophenyl)ethyl]carbamate 353c (13 mg, 0.02 mmol, 1.0 eq) in 4M HCl in MeOH (2.0 mL, 8.0 mmol) and the reaction mixture was stirred at 60° C. for 10 min. The resulting mixture was concentrated under reduced pressure. The crude residue was dissolved into MeOH (2.0 mL) and adjusted to pH 8 with NH₃·H₂O and the resulting mixture was purified by prep-HPLC [Column: YMC-Actus Triart C18, 30*150 mm, 5 μm; Mobile Phase A: Water 10 mmol/L NH₄HCO₃+0.1% NH₃·H₂O, Mobile Phase B: ACN; Flow rate: 30 mL/min; Gradient: 15% B to 60% B in 10 min, 55% B; Wave Length: 254 nm]: to afford (1R,2S,3R,5R)-3-(4-amino-5-(4-benzylthiazol-2-yl)-2-chloro-7H-pyrrolo[2,3-d]pyrimidin-7-yl)-5-(((3-((4-fluorophenethyl)amino)propyl)(methyl)amino)methyl)cyclopentane-1,2-diol 647 (4.4 mg, 38%) as an off-white solid. LCMS [ZORBAX SB-Aq, 50*4.6 mm, 1.8 μm, Mobile Phase A: Water/0.02% TFA, Mobile Phase B: Acetonitrile, 5%-95% B-3 min, 1.50 L/min, ES, m/z]: T_{R}=1.43 min; [M+H]⁺: 664, 666. ¹H NMR (400 MHZ, DMSO-d₆) δ9.43 (s, 1H), 8.15 (s, 1H), 7.83 (s, 1H), 7.37-7.27 (m, 4H), 7.25-7.19 (m, 2H), 7.18-7.11 (m, 2H), 7.02 (t, J=8.8 Hz, 2H), 4.88-4.77 (m, 2H), 4.68 (br s, 1H), 4.33-4.24 (m, 1H), 4.07 (s, 2H), 3.75-3.67 (m, 1H), 2.73-2.59 (m, 4H), 2.49-2.40 (m, 3H), 2.37-2.30 (m, 1H), 2.29-2.22 (m, 2H), 2.21-2.01 (m, 5H), 1.59-1.39 (m, 3H).

### Synthesis of (1R,2S,3R,5R)-3-(4-amino-7H-pyrrolo[2,3-d]pyrimidin-7-yl)-5-(3-(pyridin-3-yl)phenyl)cyclopentane-1,2-diol (649)

### (3aR,6R,6aR)-2,2-dimethyl-6-(3-(pyridin-3-yl)phenyl)tetrahydro-4H-cyclopenta[d][1,3]dioxol-4-one (32d)

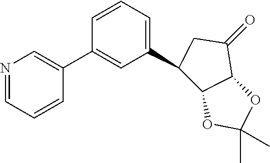 [SMILES: CC1(C)O[C@@H]2[C@@H](c3cccc(-c4cccnc4)c3)CC(=O)[C@@H]2O1]

Into a 40 mL vial were added (3aR,6aR)-2,2-dimethyl-3aH,6aH-cyclopenta[d][1,3]dioxol-4-one (0.50 g, 3.2 mmol, 1.0 eq), 3-(pyridin-3-yl)phenylboronic acid (0.77 g, 3.9 mmol, 1.2 eq), cyclooctadiene rhodium chloride dimer (0.16 g, 0.3 mmol, 0.1 eq), KOH (0.54 g, 9.7 mmol, 3.0 eq) and CH₃OH (8.0 mL) at room temperature under N₂ atmosphere. The reaction was irradiated with microwave radiation for 2 h at 50° C. The mixture was allowed to cool to room temperature. The resulting mixture was extracted with AcOEt (3×20 mL). The combined organic layers were washed with brine (2×20 mL) and dried over anhydrous Na₂SO₄. After filtration, the filtrate was concentrated under reduced pressure. The residue was dissolved in DCM (3.0 mL) and purified by silica gel column chromatography (70:30 Petroleum ether/AcOEt) to afford (3aR,6R,6aR)-2,2-dimethyl-6-(3-(pyridin-3-yl)phenyl)tetrahydro-4H-cyclopenta[d][1,3]dioxol-4-one 32d (200 mg, 20%) as a brown solid. LCMS (conditions L-column3 ODS, 50*3.0 mm, 3.0 μm, Mobile Phase A: Water/5 mM NH₄HCO₃+0.05% Ammonia, Mobile Phase B: Acetonitrile/5% water, 2%-100% B-2.5 min (+), 1.50 L/min): T_{R}=1.47 min, ES m/z [M+H]⁺: 310. ¹H NMR (400 MHZ, Chloroform-d) δ 8.82 (d, J=2.4 Hz, 1H), 8.64-8.52 (m, 1H), 7.90-7.79 (m, 1H), 7.49-7.38 (m, 3H), 7.37-7.33 (m, 1H), 7.32-7.17 (m, 1H), 4.79-4.70 (m, 1H), 4.32-4.19 (m, 1H), 3.63-3.44 (m, 1H), 2.36-2.23 (m, 1H), 2.14-1.99 (m, 1H), 1.38 (s, 3H), 1.28 (s, 3H).

### (3aS,4S,6R,6aR)-2,2-dimethyl-6-(3-(pyridin-3-yl)phenyl)tetrahydro-4H-cyclopenta[d][1,3]dioxol-4-ol (33d)

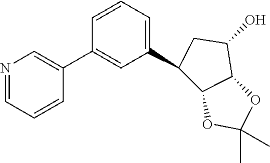 [SMILES: CC1(C)O[C@@H]2[C@H](O1)[C@@H](c1cccc(-c3cccnc3)c1)C[C@@H]2O]

Into a 40 mL vial were added (3aR,6R,6aR)-2,2-dimethyl-6-[3-(pyridin-3-yl)phenyl]-tetrahydrocyclopenta[d][1,3]dioxol-4-one 32d (0.20 g, 0.6 mmol, 1.0 eq), NaBH₄ (50 mg, 1.3 mmol, 2.0 eq) and MeOH (4.0 mL), and the reaction stirred at room temperature for 15 min. The resulting mixture was concentrated under reduced pressure. The residue was dissolved in DCM (2.0 mL) and purified by Prep-TLC (60:40 Petroleum ether/AcOEt) to afford (3aS,45,6R,6aR)-2,2-dimethyl-6-(3-(pyridin-3-yl)phenyl)tetrahydro-4H-cyclopenta[d][1,3]dioxol-4-ol 33d (140 mg, 93%) as light yellow oil. LCMS (conditions L-column3 ODS, 50*3.0 mm, 3.0 μm, Mobile Phase A: Water/5 mM NH₄HCO₃+0.05% Ammonia, Mobile Phase B: Acetonitrile/5% water, 2%-100% B-2.5 min (+), 1.50 L/min): T_{R}=1.35 min, ES m/z [M+H]⁺: 312.

### 4-Chloro-7-((3aS,4R,6R,6aR)-2,2-dimethyl-6-(3-(pyridin-3-yl)phenyl)tetrahydro-4H-cyclopenta[d][1,3]dioxol-4-yl)-7H-pyrrolo [2,3-d]pyrimidine (34d)

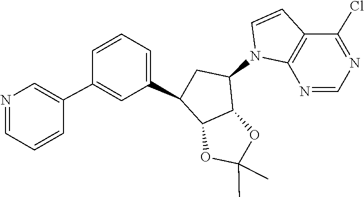 [SMILES: CC1(C)O[C@@H]2[C@H](O1)[C@@H](c1cccc(-c3cccnc3)c1)C[C@H]2n1ccc2c(Cl)ncnc21]

To a stirred solution of 4-chloro-7H-pyrrolo[2,3-d]pyrimidine 33d (0.14 g, 0.5 mmol, 1.0 eq), PPh₃ (0.25 g, 1.0 mmol, 2.0 eq) and (3aS,4S,6R,6aR)-2,2-dimethyl-6-(3-(pyridin-3-yl)phenyl)tetrahydro-4H-cyclopenta[d][1,3]dioxol-4-ol (0.11 g, 0.7 mmol, 1.5 eq) in toluene (3.0 mL) was added DBAD (0.22 g, 1.0 mmol, 2.0 eq) in toluene (2.0 mL) dropwise at 0° C. The reaction was stirred at 60° C. for 4 h. The mixture was allowed to cool to room temperature. The resulting mixture was concentrated under vacuum. The residue was dissolved in H₂O. The resulting mixture was extracted with AcOEt (2×10.0 mL). The combined organic layers were dried over anhydrous Na₂SO₄. After filtration, the filtrate was concentrated under reduced pressure. The residue was purified by Prep-TLC (50:50 Petroleum ether/AcOEt) to afford 4-chloro-7-((3aS,4R,6R,6aR)-2,2-dimethyl-6-(3-(pyridin-3-yl)phenyl)tetrahydro-4H-cyclopenta[d][1,3]dioxol-4-yl)-7H-pyrrolo[2,3-d]pyrimidine 34d (70 mg, 35%) as light yellow oil. LCMS (conditions L-column3 ODS, 50*3.0 mm, 3.0 μm, Mobile Phase A: Water/5 mM NH₄HCO₃+0.05% Ammonia, Mobile Phase B: Acetonitrile/5% water, 2%-100% B-2 min 1.50 L/min): T_{R}=1.53 min, ES m/z [M+H]⁺: 447, 449.

### 7-((3aS,4R,6R,6aR)-2,2-dimethyl-6-(3-(pyridin-3-yl)phenyl)tetrahydro-4H-cyclopenta[d][1,3]dioxol-4-yl)-7H-pyrrolo[2,3-d]pyrimidin-4-amine (35d)

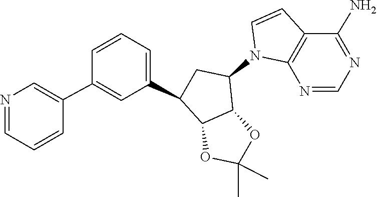 [SMILES: CC1(C)O[C@@H]2[C@H](O1)[C@@H](c1cccc(-c3cccnc3)c1)C[C@H]2n1ccc2c(N)ncnc21]

Into a 40 mL vial were added 4-chloro-7-((3aS,4R,6R,6aR)-2,2-dimethyl-6-(3-(pyridin-3-yl)phenyl)tetrahydro-4H-cyclopenta[d][1,3]dioxol-4-yl)-7H-pyrrolo[2,3-d]pyrimidine 34d (70 mg, 0.16 mmol, 1.0 eq) and 7M NH₃ in MeOH (3.0 mL) at room temperature, and the mixture was stirred at 80° C. for 24 h, The resulting mixture was concentrated under reduced pressure to afford 7-((3aS,4R,6R,6aR)-2,2-dimethyl-6-(3-(pyridin-3-yl)phenyl)tetrahydro-4H-cyclopenta[d][1,3]dioxol-4-yl)-7H-pyrrolo[2,3-d]pyrimidin-4-amine (20 mg, crude). This was used in the next step directly without further purification. LCMS (conditions L-column3 ODS, 50*3.0 mm, 3.0 μm, Mobile Phase A: Water/5 mM NH₄HCO₃+0.05% Ammonia, Mobile Phase B: Acetonitrile/5% water, 2%-100% B-2.5 min (+), 1.50 L/min): T_{R}=1.40 min, ES m/z [M+H]⁺: 428.

### (1R,2S,3R,5R)-3-(4-amino-7H-pyrrolo[2,3-d]pyrimidin-7-yl)-5-(3-(pyridin-3-yl)phenyl)cyclopentane-1,2-diol (649)

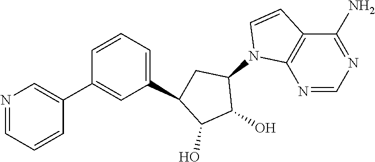 [SMILES: Nc1ncnc2c1ccn2[C@@H]1C[C@H](c2cccc(-c3cccnc3)c2)[C@@H](O)[C@H]1O]

Into a 40 mL vial was placed a solution of 7-((3aS,4R,6R,6aR)-2,2-dimethyl-6-(3-(pyridin-3-yl)phenyl)tetrahydro-4H-cyclopenta[d][1,3]dioxol-4-yl)-7H-pyrrolo[2,3-d]pyrimidin-4-amine 35d (30 mg, 0.070 mmol, 1.0 eq) in 4M HCl in MeOH (2.0 mL). The mixture was stirred for 30 min at 40° C., then concentrated under reduced pressure and basified to pH>8 with NH₃·H₂O. The residue was purified by Prep-HPLC with the following conditions (Column: YMC-Actus Triart C18, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 24% B to 60% B in 10 min, 60% B; Wavelength: 220 nm) to afford (1R,2S,3R,5R)-3-(4-amino-7H-pyrrolo[2,3-d]pyrimidin-7-yl)-5-(3-(pyridin-3-yl)phenyl)cyclopentane-1,2-diol 649 (5.0 mg, 19%) as a white solid. LCMS (ZORBAX SB-Aq, 50*4.6 mm, 1.8 μm, Mobile Phase A: Water/0.02% TFA, Mobile Phase B: Acetonitrile, 5%-95% B-3 min, 1.50 L/min): T_{R}=0.62 min, ES m/z [M+H]⁺: 388. ¹H NMR (300 MHZ, DMSO-d₆+D₂O) δ 8.90 (d, J=2.4 Hz, 1H), 8.57 (dd, J=4.8, 1.6 Hz, 1H), 8.10 (dt, J=8.3, 1.9 Hz, 1H), 8.05 (s, 1H), 7.75 (s, 1H), 7.63-7.53 (m, 1H), 7.56-7.41 (m, 3H), 7.39 (d, J=3.6 Hz, 1H), 6.58 (d, J=3.5 Hz, 1H), 4.99-4.84 (m, 1H), 4.32 (t, J=6.6 Hz, 1H), 4.15 (t, J=6.8 Hz, 1H), 3.27-3.12 (m, 1H), 2.48-2.33 (m, 1H), 2.11 (q, J=11.7 Hz, 1H).

### Synthesis of (1R,2S,3R,5R)-3-[4-amino-2-chloro-5-(1,3-thiazol-2-yl)pyrrolo[2,3-d]pyrimidin-7-yl]-5-{[(3-{[2-(4-fluorophenyl)ethyl]amino}propyl)(methyl)amino]methyl}cyclopentane-1,2-diol (648)

### tert-Butyl-N-{[(3aR,4R,6R,6aS)-6-[4-amino-2-chloro-5-(1,3-thiazol-2-yl)pyrrolo[2,3-d]pyrimidin-7-yl]-2,2-dimethyl-tetrahydro-3aH-cyclopenta[d][1,3]dioxol-4-yl]methyl}carbamate (354c)

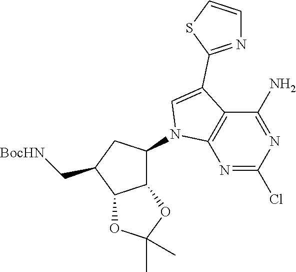 [SMILES: CC(C)(C)OC(=O)NC[C@H]1C[C@@H](n2cc(-c3nccs3)c3c(N)nc(Cl)nc32)[C@@H]2OC(C)(C)O[C@H]12]

To a solution of tert-butyl-N-{[(3aR,4R,6R,6aS)-6-{4-amino-2-chloro-5-iodopyrrolo[2,3-d]pyrimidin-7-yl}-2,2-dimethyl-tetrahydro-3aH-cyclopenta[d][1,3]dioxol-4-yl]methyl}carbamate 349c (0.11 g, 0.20 mmol, 1.0 eq) and Pd (PPh₃) 2Cl₂ (7.0 mg, 0.01 mmol, 0.05 eq) in toluene (3.0 mL), at room temperature under N₂, was added 2-(tributylstannyl)-1,3-thiazole (0.14 g, 0.40 mmol, 2.0 eq) and the reaction mixture was stirred at 120° C. for 2 h. The resulting mixture was allowed to cool to room temperature and was then concentrated under reduced pressure. The crude residue was dissolved in DCM (3.0 mL) and was then purified by silica gel column chromatography (3:2 Petroleum ether/THF) to afford tert-butyl-N-{[(3aR,4R,6R,6aS)-6-[4-amino-2-chloro-5-(1,3-thiazol-2-yl)pyrrolo[2,3-d]pyrimidin-7-yl]-2,2-dimethyl-tetrahydro-3aH-cyclopenta[d][1,3]dioxol-4-yl]methyl}carbamate 354c (60 mg, 59%) as a white solid. LCMS [conditions Cortecs C18+, 50*3.0 mm, 2.7 μm, Mobile Phase A: Water/0.05% TFA, Mobile Phase B: Acetonitrile, 2%-100% B-3 min (+), 1.50 L/min, ES, m/z]: T_{R}=1.65 min; [M+H]⁺: 521, 523.

### (1R,2S,3R,5R)-3-[4-Amino-2-chloro-5-(1,3-thiazol-2-yl)pyrrolo[2,3-d]pyrimidin-7-yl]-5-(aminomethyl)cyclopentane-1,2-diol (355c)

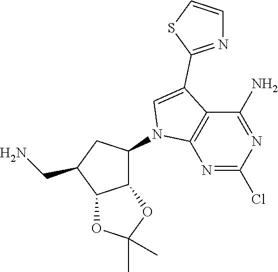 [SMILES: CC1(C)O[C@@H]2[C@@H](CN)C[C@@H](n3cc(-c4nccs4)c4c(N)nc(Cl)nc43)[C@@H]2O1]

To a solution of tert-butyl-N-{[(3aR,4R,6R,6aS)-6-[4-amino-2-chloro-5-(1,3-thiazol-2-yl)pyrrolo[2,3-d]pyrimidin-7-yl]-2,2-dimethyl-tetrahydro-3aH-cyclopenta[d][1,3]dioxol-4-yl]methyl}carbamate 354c (60 mg, 0.11 mmol, 1.0 eq) in DCM (1.0 mL) was added TFA (1.0 mL) and the reaction mixture was stirred for 1 h at room temperature. The resulting mixture was concentrated under reduced pressure to afford (1R,2S,3R,5R)-3-[4-amino-2-chloro-5-(1,3-thiazol-2-yl)pyrrolo[2,3-d]pyrimidin-7-yl]-5-(aminomethyl)cyclopentane-1,2-diol HCl salt 355c (40 mg, crude) which was used in the next step without purification. LCMS [conditions L-column3 ODS, 50*3.0 mm, 3.0 μm, Mobile PhaseA: Water/5 mM NH₄HCO₃+0.05% Ammonia, Mobile Phase B: Acetonitrile/5% water, 2%-100% B-2.5 min (+), 1.50 L/min, ES, m/z]: T_{R}=1.57 min; [M+H]⁺: 381, 383.

### tert-Butyl-N-[3-({[(1R,2R,3S,4R)-4-[4-amino-2-chloro-5-(1,3-thiazol-2-yl)pyrrolo[2,3-d]pyrimidin-7-yl]-2,3-dihydroxycyclopentyl]methyl}(methyl)amino)propyl]-N-[2-(4-fluorophenyl)ethyl]carbamate (356c)

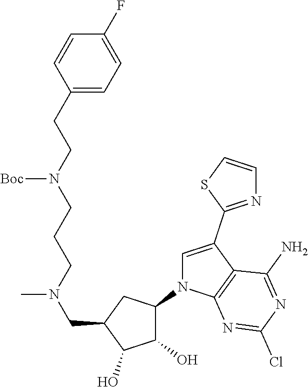 [SMILES: CN(CCCN(CCc1ccc(F)cc1)C(=O)OC(C)(C)C)C[C@H]1C[C@@H](n2cc(-c3nccs3)c3c(N)nc(Cl)nc32)[C@H](O)[C@@H]1O]

To a solution of (1R,2S,3R,5R)-3-[4-amino-2-chloro-5-(1,3-thiazol-2-yl)pyrrolo[2,3-d]pyrimidin-7-yl]-5-(aminomethyl)cyclopentane-1,2-diol HCl salt 355c (40 mg, 0.10 mmol, 1.0 eq) in DCM (0.5 mL) was added tert-butyl-N-[2-(4-fluorophenyl)ethyl]-N-(3-oxopropyl)carbamate 188c (46.5 mg, 0.16 mmol, 1.5 eq), DIEA (27.1 mg, 0.21 mmol, 2.0 eq) and NaBH (OAc) 3 (42 mg, 0.20 mmol, 2.0 eq) and the resulting mixture was stirred for 20 min at room temperature. HCHO (4.5 mg, 0.15 mmol, 1.5 eq) was then added and the resulting mixture was stirred for 30 min at room temperature before adding MeOH (0.5 mL) and then NaBH₄ (3.18 mg, 0.084 mmol, 0.8 eq), at room temperature, and the reaction mixture was stirred for 10 min at room temperature. The resulting mixture was concentrated under reduced pressure, dissolved in MeOH (1.0 mL) and the crude residue was purified by Prep-HPLC [Column: Welch Ultimate XB-C18, 50*250 mm, 10 μm; Mobile Phase A: Water/5 mM NH₄HCO₃, Mobile Phase B: ACN; Flow rate: 90 mL/min; Gradient: 5% B-5% B-2 min, 10% B-60% B-12 mim]: to afford tert-butyl-N-[3-({[(1R,2R,3S,4R)-4-[4-amino-2-chloro-5-(1,3-thiazol-2-yl)pyrrolo[2,3-d]pyrimidin-7-yl]-2,3-dihydroxycyclopentyl]methyl}(methyl)amino)propyl]-N-[2-(4-fluorophenyl)ethyl]carbamate 356c (15 mg, 21%) as an off-white solid. LCMS [conditions L-column3 ODS, 50*3.0 mm, 3.0 μm, Mobile PhaseA: Water/5 mM NH₄HCO₃+0.05% Ammonia, Mobile Phase B: Acetonitrile/5% water, 2%-100% B-3 min (+), 1.50 L/min, ES, m/z]: T_{R}=2.22 min; [M+H]⁺: 674, 676.

### (1R,2S,3R,5R)-3-[4-Amino-2-chloro-5-(1,3-thiazol-2-yl)pyrrolo[2,3-d]pyrimidin-7-yl]-5-{[(3-{[2-(4-fluorophenyl)ethyl]amino}propyl)(methyl)amino]methyl}cyclopentane-1,2-diol (648)

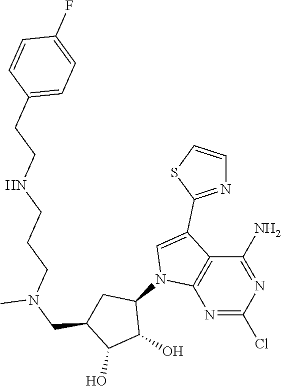 [SMILES: CN(CCCNCCc1ccc(F)cc1)C[C@H]1C[C@@H](n2cc(-c3nccs3)c3c(N)nc(Cl)nc32)[C@H](O)[C@@H]1O]

To tert-butyl-N-[3-({[(1R,2R,3S,4R)-4-[4-amino-2-chloro-5-(1,3-thiazol-2-yl)pyrrolo[2,3-d]pyrimidin-7-yl]-2,3-dihydroxycyclopentyl]methyl}(methyl)amino)propyl]-N-[2-(4-fluorophenyl)ethyl]carbamate 356c (15 mg, 0.02 mmol, 1.0 eq) was added 4M HCl in MeOH (2.0 mL, 8.0 mmol) and the reaction mixture was stirred at 60° C. for 10 min. The resulting mixture was concentrated under vacuum, the residue was dissolved in MeOH (2.0 mL) and the solution was adjusted to pH 8 with NH₃·H₂O. The crude mixture was purified by Prep-HPLC [Column: YMC-Actus Triart C18, 30*150 mm, 5 μm; Mobile Phase A: Water 10 mmol/L NH₄HCO₃+0.1% NH₃·H₂O, Mobile Phase B: ACN; Flow rate: 30 ml/min; Gradient: 15% B to 60% B in 10 min, 55% B; Wave Length: 254 nm]: to afford (1R,2S,3R,5R)-3-[4-amino-2-chloro-5-(1,3-thiazol-2-yl)pyrrolo[2,3-d]pyrimidin-7-yl]-5-{[(3-{[2-(4-fluorophenyl)ethyl]amino}propyl)(methyl)amino]methyl}cyclopentane-1,2-diol 648 (4.0 mg, 31%) as an off-white solid. LCMS [ZORBAX SB-Aq, 50*4.6 mm, 1.8 μm, Mobile Phase A: Water/0.02% TFA, Mobile Phase B: Acetonitrile, 5%-95% B-3 min, 1.50 L/min, ES, m/z]: T_{R}=1.25 min; [M+H]⁺: 574, 576. ¹H NMR (400 MHZ, DMSO-d) δ 9.53 (s, 1H), 8.21 (s, 1H), 7.94 (s, 1H), 7.83 (d, J=3.5 Hz, 1H), 7.65 (d, J=3.4 Hz, 1H), 7.20-7.13 (m, 2H), 7.03 (t, J=8.0 Hz, 2H), 4.90-4.81 (m, 2H), 4.68 (br s, 1H), 4.38-4.27 (m, 1H), 3.75-3.70 (m, 1H), 2.74-2.59 (m, 2H), 2.47-2.41 (m, 6H), 2.38-2.31 (m, 1H), 2.30-2.23 (m, 1H), 2.20-2.08 (m, 5H), 1.62-1.45 (m, 3H).

### (1R,2S,3R,5R)-3-(4-amino-7H-pyrrolo[2,3-d]pyrimidin-7-yl)-5-(3-(isothiazol-4-yl)phenyl)cyclopentane-1,2-diol (650)

### (3aR,6R,6aR)-6-(3-bromophenyl)-2,2-dimethyltetrahydro-4H-cyclopenta[d][1,3]dioxol-4-one (36d)

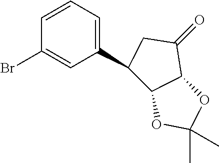 [SMILES: CC1(C)O[C@@H]2[C@@H](c3cccc(Br)c3)CC(=O)[C@@H]2O1]

Into a 40 mL vial were added (3aR,6aR)-2,2-dimethyl-3aH,6aH-cyclopenta[d][1,3]dioxol-4-one (0.50 g, 3.2 mmol, 1.0 eq), 3-bromophenylboronic acid (0.78 g, 3.9 mmol, 1.2 eq), cyclooctadiene rhodium chloride dimer (0.16 g, 0.3 mmol, 0.1 eq), KOH (0.55 g, 9.8 mmol, 3.0 eq) and CH₃OH (8.0 mL) at room temperature under N₂ atmosphere. The reaction was irradiated with microwave radiation for 2 h at 40° C. The mixture was allowed to cool to room temperature. The resulting mixture was extracted with AcOEt (3×20 mL). The combined organic layers were washed with brine and dried over anhydrous Na₂SO₄. After filtration, the filtrate was concentrated under reduced pressure. The residue was dissolved in DCM (3.0 mL) and purified by silica gel column chromatography (70:30 Petroleum ether/THF) to afford (3aR,6R,6aR)-6-(3-bromophenyl)-2,2-dimethyltetrahydro-4H-cyclopenta[d][1,3]dioxol-4-one 36d (120 mg, 12%) as a white solid. 1H NMR (300 MHZ, Chloroform-d) 87.46-7.40 (m, 1H), 7.34-7.29 (m, 1H), 7.22 (d, J=7.9 Hz, 1H), 7.09-7.03 (m, 1H), 4.76-4.69 (m, 1H), 4.42-4.36 (m, 1H), 3.69-3.60 (m, 1H), 3.17-3.03 (m, 1H), 2.58-2.47 (m, 1H), 1.37 (s, 3H), 1.34 (s, 3H).

### (3aR,6R,6aR)-6-(3-(isothiazol-4-yl)phenyl)-2,2-dimethyltetrahydro-4H-cyclopenta[d][1,3]dioxol-4-one (38d)

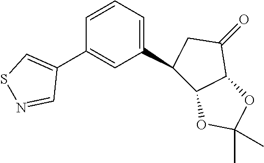 [SMILES: CC1(C)O[C@@H]2[C@@H](c3cccc(-c4cnsc4)c3)CC(=O)[C@@H]2O1]

Into a 8 mL vial were added (3aR,6R,6aR)-6-(3-bromophenyl)-2,2-dimethyl-tetrahydrocyclopenta[d][1,3]dioxol-4-one 36d (0.10 g, 0.3 mmol, 1.0 eq), 4-(4,4,5,5-tetramethyl-1,3,2-dioxaborolan-2-yl)-1,2-thiazole mixture 37d (0.13 g, 0.6 mmol, 2.0 eq) [WO2015100117], Pd(dtbpf)Cl₂ (21.0 mg, 0.032 mmol, 0.1 eq), K₂CO₃ (0.13 g, 1.0 mmol, 3.0 eq), dioxane (2.0 mL) and H₂O (0.2 mL) at room temperature, The reaction was stirred at 80° C. for 2 h under N₂ atmosphere. The mixture was allowed to cool to room temperature, then concentrated under reduced pressure. The residue was dissolved in H₂O (10.0 mL). The resulting mixture was extracted with EtOAc (3×10 mL). The combined organic layers were washed with brine and dried over anhydrous Na₂SO₄. After filtration, the filtrate was concentrated under reduced pressure. The residue was dissolved in DCM (3.0 mL) and purified by silica gel column chromatography (70:30 Petroleum ether/EtOAc) to afford (3aR,6R,6aR)-6-(3-(isothiazol-4-yl)phenyl)-2,2-dimethyltetrahydro-4H-cyclopenta[d][1,3]dioxol-4-one 38d (70 mg, 70%) as a white solid. LCMS (conditions L-column3 ODS, 50*3.0 mm, 3.0 μm, Mobile Phase A: Water/5 mM NH₄HCO₃+0.05% Ammonia, Mobile Phase B: Acetonitrile/5% water, 2%-100% B-2.5 min (+), 1.50 L/min): T_{R}=1.55 min, ES m/z [M+H]⁺: 316.

### (3aS,4S,6R,6aR)-6-(3-(isothiazol-4-yl)phenyl)-2,2-dimethyltetrahydro-4H-cyclopenta[d][1,3]dioxol-4-ol (39d)

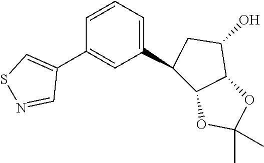 [SMILES: CC1(C)O[C@@H]2[C@H](O1)[C@@H](c1cccc(-c3cnsc3)c1)C[C@@H]2O]

Into a 40 mL vial were added (3aR,6R,6aR)-6-(3-(isothiazol-4-yl)phenyl)-2,2-dimethyltetrahydro-4H-cyclopenta[d][1,3]dioxol-4-one 38d (65.0 mg, 0.2 mmol, 1.0 eq), NaBH₄ (32.0 mg, 0.8 mmol, 4.0 eq) and MeOH (2.0 mL) at room temperature, and the solution stirred for 15 mins. The reaction was quenched with H₂O at room temperature. The crude product was purified by reverse phase flash with the following conditions (Column: Welch Utimate AQ-C18, 50*250 mm*10 μm; Mobile Phase A: Water (10 mmol/L NH₄HCO₃+0.1% NH₃·H₂O), Mobile Phase B: ACN; Flow rate: 80 mL/min; Gradient: 5% B to 65% B in 15 min, 65% B; Wavelength: 220 nm) to afford (3aS,4S,6R,6aR)-6-(3-(isothiazol-4-yl)phenyl)-2,2-dimethyltetrahydro-4H-cyclopenta[d][1,3]dioxol-4-ol 39 (50 mg, 76%) as an off white solid. LCMS (conditions L-column3 ODS, 50*3.0 mm, 3.0 μm, Mobile Phase A: Water/5 mM NH₄HCO₃+0.05% Ammonia, Mobile Phase B: Acetonitrile/5% water, 2%-100% B-3 min (+), 1.50 L/min): T_{R}=1.30 min, ES m/z [M+H]⁺: 318.

### 4-(3-((3aR,4R,6R,6aS)-6-(4-chloro-7H-pyrrolo[2,3-d]pyrimidin-7-yl)-2,2-dimethyltetrahydro-4H-cyclopenta[d][1,3]dioxol-4-yl)phenyl) isothiazole (40d)

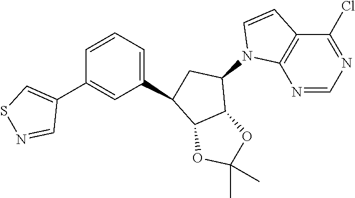 [SMILES: CC1(C)O[C@@H]2[C@H](O1)[C@@H](c1cccc(-c3cnsc3)c1)C[C@H]2n1ccc2c(Cl)ncnc21]

To a stirred solution of (3aS,4S,6R,6aR)-6-(3-(isothiazol-4-yl)phenyl)-2,2-dimethyltetrahydro-4H-cyclopenta[d][1,3]dioxol-4-ol 39d (50 mg, 0.2 mmol, 1.1 eq), 4-chloro-7H-pyrrolo[2,3-d]pyrimidine (26 mg, 0.17 mmol, 1.0 eq) and PPh₃ (90.0 mg, 0.35 mmol, 2.0 eq) in toluene (2.0 mL) was added DBAD (79.0 mg, 0.34 mmol, 2.0 eq) in toluene (1.0 mL) dropwise at 0° C. under N₂ atmosphere. The reaction was stirred at 50° C. for 3 h. The mixture was allowed to cool to room temperature, then concentrated under reduced pressure. The residue was dissolved in H₂O (10.0 mL) and the resulting mixture was extracted with AcOEt (3×10.0 mL). The combined organic layers were washed with brine and dried over anhydrous Na₂SO₄. After filtration, the filtrate was concentrated under reduced pressure. The residue was dissolved in DCM (3.0 mL) and purified by silica gel column chromatography (50:50 Petroleum ether/THF) to afford 4-(3-((3aR,4R,6R,6aS)-6-(4-chloro-7H-pyrrolo[2,3-d]pyrimidin-7-yl)-2,2-dimethyltetrahydro-4H-cyclopenta[d][1,3]dioxol-4-yl)phenyl) isothiazole 40d (60 mg, 78%) as a light yellow solid. LCMS (conditions L-column3 ODS, 50*3.0 mm, 3.0 μm, Mobile Phase A: Water/5 mM NH₄HCO₃+0.05% Ammonia, Mobile Phase B: Acetonitrile/5% water, 2%-100% B-2.5 min (+), 1.50 L/min): T_{R}=1.80 min, ES m/z [M+H]⁺: 453, 455.

### 7-((3aS,4R,6R,6aR)-6-(3-(isothiazol-4-yl)phenyl)-2,2-dimethyltetrahydro-4H-cyclopenta[d][1,3]dioxol-4-yl)-N-(4-methoxybenzyl)-7H-pyrrolo[2,3-d]pyrimidin-4-amine (41d)

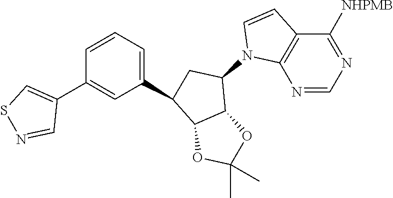

Into an 8 mL vial were added 4-(3-((3aR,4R,6R,6aS)-6-(4-chloro-7H-pyrrolo[2,3-d]pyrimidin-7-yl)-2,2-dimethyltetrahydro-4H-cyclopenta[d][1,3]dioxol-4-yl)phenyl) isothiazole 40d (50 mg, 0.1 mmol, 1.0 eq), 4-methoxy-benzenemethanamine (30.3 mg, 0.2 mmol, 2.0 eq), Et₃N (34 mg, 0.3 mmol, 3.0 eq) and EtOH (3.0 mL) at room temperature. The reaction was stirred at 80° C. for 2 h. The mixture was allowed to cool to room temperature, then concentrated under reduced pressure. The residue was dissolved in DCM (2.0 mL) and purified by silica gel column chromatography (50:50 Petroleum ether/EtOAc) to afford 7-((3aS,4R,6R,6aR)-6-(3-(isothiazol-4-yl)phenyl)-2,2-dimethyltetrahydro-4H-cyclopenta[d][1,3]dioxol-4-yl)-N-(4-methoxybenzyl)-7H-pyrrolo[2,3-d]pyrimidin-4-amine 41d (45 mg, 73%) as a light yellow solid. LCMS (conditions L-column3 ODS, 50*3.0 mm, 3.0 μm, Mobile Phase A: Water/5 mM NH₄HCO₃+0.05% Ammonia, Mobile Phase B: Acetonitrile/5% water, 2%-100% B-2.5 min (+), 1.50 L/min): T_{R}=1.48 min, ES m/z [M+H]⁺: 554.

### (1R,2S,3R,5R)-3-(4-amino-7H-pyrrolo[2,3-d]pyrimidin-7-yl)-5-(3-(isothiazol-4-yl)phenyl)cyclopentane-1,2-diol (650)

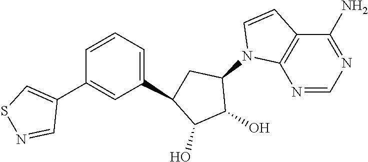 [SMILES: Nc1ncnc2c1ccn2[C@@H]1C[C@H](c2cccc(-c3cnsc3)c2)[C@@H](O)[C@H]1O]

Into a 40 mL vial, was placed a solution of 7-((3aS,4R,6R,6aR)-6-(3-(isothiazol-4-yl)phenyl)-2,2-dimethyltetrahydro-4H-cyclopenta[d][1,3]dioxol-4-yl)-N-(4-methoxybenzyl)-7H-pyrrolo[2,3-d]pyrimidin-4-amine 41d (45 mg, 0.081 mmol, 1.0 eq) in TFA (3.0 mL, 0.1 mmol, 1.0 eq). The mixture was stirred for 2 h at 60° C., and the resulting mixture concentrated under reduced pressure. The residue was purified by Prep-HPLC (Column: XBridge Prep C18 OBD Column, 19*150 mm, 5 μm; Mobile Phase A: Water (10 mmol/L NH₄HCO₃+0.1% NH₃·H₂O), Mobile Phase B: ACN; Flow rate: 20 mL/min; Gradient: 10% B to 50% B in 8 min, 50% B; Wavelength: 254/220 nm) to afford (1R,2S,3R,5R)-3-(4-amino-7H-pyrrolo[2,3-d]pyrimidin-7-yl)-5-(3-(isothiazol-4-yl)phenyl)cyclopentane-1,2-diol 650 (13.8 mg, 30%) as a white solid. LCMS (Cortecs C18+, 50*3.0 mm, 2.7 μm, Mobile Phase A: Water/0.02% TFA, Mobile Phase B: Acetonitrile, 5%-95% B-3 min, 1.50 L/min): T_{R}=0.85 min, ES m/z [M+H]⁺: 394. ¹H NMR (300 MHZ, DMSO-d₆) δ 9.37 (s, 1H), 9.08 (s, 1H), 8.08 (s, 1H), 7.80 (s, 1H), 7.73-7.60 (m, 1H), 7.49-7.33 (m, 3H), 6.95 (s, 2H), 6.59 (d, J=3.5 Hz, 1H), 4.99 (d, J=5.8 Hz, 1H), 4.95-4.88 (m, 2H), 4.38-4.25 (m, 1H), 4.20-4.12 (m, 1H), 3.23-3.08 (m, 1H), 2.47-2.35 (m, 1H), 2.15-2.01 (m, 1H).

### Synthesis of (1R,2S,3R,5R)-3-[5-(4-benzyl-1,3-thiazol-2-yl)-2-chloropyrrolo[2,3-d]pyrimidin-7-yl]-5-[1-(2,2,2-trifluoroethyl)-5,6-dihydro-2H-pyridin-3-yl]cyclopentane-1,2-diol (651) (1R,2S,3R,5R)-3-[5-(4-Benzyl-1,3-thiazol-2-yl)-2-chloropyrrolo[2,3-d]pyrimidin-7-yl]-5-[1-(2,2,2-trifluoroethyl)-5,6-dihydro-2H-pyridin-3-yl]cyclopentane-1,2-diol (651)

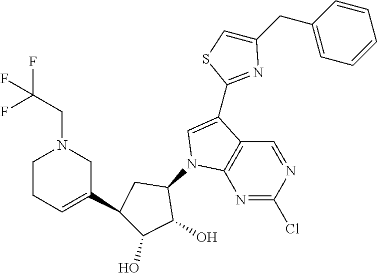 [SMILES: O[C@@H]1[C@H](O)[C@@H](C2=CCCN(CC(F)(F)F)C2)C[C@H]1n1cc(-c2nc(Cc3ccccc3)cs2)c2cnc(Cl)nc21]

Into an 8 mL vial were added (1R,2S,3R,5R)-3-[5-(4-benzyl-1,3-thiazol-2-yl)-2-chloropyrrolo[2,3-d]pyrimidin-7-yl]-5-(1,2,5,6-tetrahydropyridin-3-yl)cyclopentane-1,2-diol (80 mg, 0.15 mmol, 1.0 eq), 2,2,2-trifluoroethyl trifluoromethanesulfonate (73.1 mg, 0.3 mmol, 2.0 eq) and DIEA (40.7 mg, 0.3 mmol, 2.0 eq) in DCM (2 mL) at room temperature. The reaction was stirred for 1 h, then concentrated. The residue was purified by Prep-HPLC [column, Xbridge Prep C18 OBD column, 5 μm, 19*150 mm; mobile phase, Water (0.03% NH₄OH) and CH₃CN (25-50% over 10 min); Detector, UV 220&254 nm]to afford (1R,2S,3R,5R)-3-[5-(4-benzyl-1,3-thiazol-2-yl)-2-chloropyrrolo[2,3-d]pyrimidin-7-yl]-5-[1-(2,2,2-trifluoroethyl)-5,6-dihydro-2H-pyridin-3-yl]cyclopentane-1,2-diol 651 (14.8 mg, 16%) as a white solid. LCMS (conditions: Cortecs C18+, 50*3.0 mm, 2.7 μm, Mobile Phase A: Water/0.02% TFA, Mobile Phase B: Acetonitrile, 5%-95% B 3 min, 1.50 ml/min): T_{R}=1.66 min; ES m/z [M+H]⁺: 590, 592. ¹H NMR (300 MHZ, DMSO-d₆) δ 9.32 (s, 1H), 8.61 (s, 1H), 7.44-7.16 (m, 6H), 5.68 (s, 1H), 5.01 (d, J=6.2 Hz, 1H), 4.98-4.87 (m, 1H), 4.85 (d, J=5.5 Hz, 1H), 4.24 (q, J=6.9 Hz, 1H), 4.14 (s, 2H), 3.87 (q, J=5.7 Hz, 1H), 3.30-3.08 (m, 4H), 2.72 (t, J=5.7 Hz, 2H), 2.47-2.36 (m, 1H), 2.20-2.07 (m, 3H), 1.97 (q, J=11.7 Hz, 1H).

### Synthesis of (15,2R,3R,5R)-3-(3-(1H-pyrazol-4-yl)phenyl)-5-(4-amino-7H-pyrrolo[2,3-d]pyrimidin-7-yl)cyclopentane-1,2-diol (652)

### (3aR,6R,6aR)-2,2-dimethyl-6-(3-(1-(tetrahydro-2H-pyran-2-yl)-1H-pyrazol-4-yl)phenyl)tetrahydro-4H-cyclopenta[d][1.3]dioxol-4-one (47d)

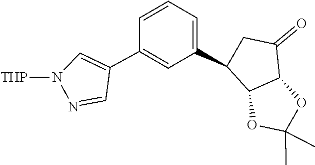

Into a 40 mL vial were added (3aR,6R,6aR)-6-(3-bromophenyl)-2,2-dimethyl-tetrahydrocyclopenta[d][1,3]dioxol-4-one (0.20 g, 0.6 mmol, 1.0 eq), 1-(oxan-2-yl)-4-(4,4,5,5-tetramethyl-1,3,2-dioxaborolan-2-yl)pyrazole (0.21 g, 0.77 mmol, 1.2 eq), Pd (dppf) Cl₂ (47.0 mg, 0.064 mmol, 0.1 eq), K₂CO₃ (0.27 g, 1.9 mmol, 3.0 eq), dioxane (3.0 mL) and H₂O (0.3 mL) at room temperature under N₂ atmosphere. The reaction was stirred at 80° C. for 2 h. The mixture was allowed to cool to room temperature, then concentrated under reduced pressure. The residue was dissolved in H₂O (20.0 mL). The resulting mixture was extracted with EtOAc (3×20.0 mL). The combined organic layers were washed with brine and dried over anhydrous Na₂SO₄. After filtration, the filtrate was concentrated under reduced pressure. The residue was purified by silica gel column chromatography (70:30 Petroleum ether/THF) to afford (3aR,6R,6aR)-2,2-dimethyl-6-(3-(1-(tetrahydro-2H-pyran-2-yl)-1H-pyrazol-4-yl)phenyl)tetrahydro-4H-cyclopenta[d][1,3]dioxol-4-one 42d (150 mg, 61%) as a yellow oil. LCMS (conditions Proshell HPH-C18, 50*3.0 mm, 2.7 μm, Mobile Phase A: Water (0.05% ammonia water), Mobile Phase B: Acetonitrile, 5%-100% B-2.5 min, 1.50 L/min): T_{R}=1.60 min, ES m/z [M+H]⁺: 383.

### (3aS,4S,6R,6aR)-2,2-dimethyl-6-(3-(1-(tetrahydro-2H-pyran-2-yl)-1H-pyrazol-4-yl)phenyl)tetrahydro-4H-cyclopenta[d][1,3]dioxol-4-ol (43d)

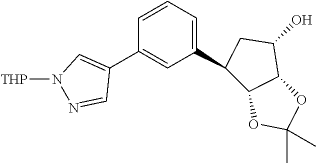

To a stirred solution of (3aR,6R,6aR)-2,2-dimethyl-6-(3-(1-(tetrahydro-2H-pyran-2-yl)-1H-pyrazol-4-yl)phenyl)tetrahydro-4H-cyclopenta[d][1,3]dioxol-4-one 42d (0.14 g, 0.4 mmol, 1.0 eq) in MeOH (2.0 mL) was added NaBH₄ (27.70 mg, 0.732 mmol, 2.0 eq) in portions at 0° C. The solution was stirred at room temperature for 15 min. The reaction was quenched with H₂O and concentrated under reduced pressure. The residue was dissolved in DCM (3.0 mL) and purified by silica gel column chromatography (60:40 Petroleum ether/AcOEt) to afford (3aS,4S,6R,6aR)-2,2-dimethyl-6-(3-(1-(tetrahydro-2H-pyran-2-yl)-1H-pyrazol-4-yl)phenyl)tetrahydro-4H-cyclopenta[d][1,3]dioxol-4-ol 43d (120 mg, 85%) as white oil. LCMS (conditions Proshell HPH-C18, 50*3.0 mm, 2.7 μm, Mobile Phase A: Water (0.05% ammonia water), Mobile Phase B: Acetonitrile, 5%-100% B-2.5 min, 1.50 L/min): T_{R}=1.48 min, ES m/z [M+H]⁺: 385.

### 4-chloro-7-((3aS,4R,6R,6aR)-2,2-dimethyl-6-(3-(1-(tetrahydro-2H-pyran-2-yl)-1H-pyrazol-4-yl)phenyl)tetrahydro-4H-cyclopenta[d][1,3]dioxol-4-yl)-7H-pyrrolo[2,3-d]pyrimidine (44d)

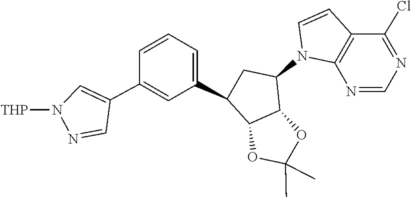

To a stirred solution of (3aS,4S,6R,6aR)-2,2-dimethyl-6-(3-(1-(tetrahydro-2H-pyran-2-yl)-1H-pyrazol-4-yl)phenyl)tetrahydro-4H-cyclopenta[d][1,3]dioxol-4-ol 43d (0.12 g, 0.3 mmol, 1.2 eq), 4-chloro-7H-pyrrolo[2,3-d]pyrimidine (40 mg, 0.26 mmol, 1.0 eq) and PPh₃ (0.13 g, 0.5 mmol, 2.0 eq) in toluene (2.0 mL) was added DBAD (0.12 g, 0.5 mmol, 2.0 eq) in toluene (2.0 mL) dropwise at 0° C. under N₂ atmosphere. The reaction was stirred at 50° C. for 4 h. The mixture was allowed to cool to room temperature, then concentrated under reduced pressure. The residue was dissolved in DCM (3.0 mL) and purified by silica gel column chromatography (50:50 Petroleum ether/THF) to afford to afford 4-chloro-7-((3aS,4R,6R,6aR)-2,2-dimethyl-6-(3-(1-(tetrahydro-2H-pyran-2-yl)-1H-pyrazol-4-yl)phenyl)tetrahydro-4H-cyclopenta[d][1,3]dioxol-4-yl)-7H-pyrrolo[2,3-d]pyrimidine 44d (120 mg, 88%) as a yellow oil. LCMS (conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-2.5 min-, 1.50 L/min): T_{R}=1.84 min, ES m/z [M+H]⁺: 520, 522.

### 7-((3aS,4R,6R,6aR)-2,2-dimethyl-6-(3-(1-(tetrahydro-2H-pyran-2-yl)-1H-pyrazol-4-yl)phenyl)tetrahydro-4H-cyclopenta[d][1,3]dioxol-4-yl)-N-(4-methoxybenzyl)-7H-pyrrolo[2,3-d]pyrimidin-4-amine (45d)

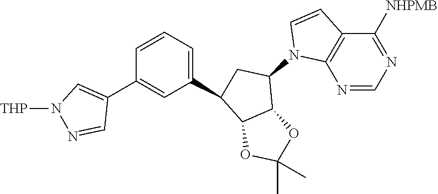

Into a 40 mL vial were added 4-chloro-7-((3aS,4R,6R,6aR)-2,2-dimethyl-6-(3-(1-(tetrahydro-2H-pyran-2-yl)-1H-pyrazol-4-yl)phenyl)tetrahydro-4H-cyclopenta[d][1,3]dioxol-4-yl)-7H-pyrrolo[2,3-d]pyrimidine 44d (0.10 mg, 0.2 mmol, 1.0 eq), 4-methoxy-benzenemethanamine (0.27 g, 1.9 mmol, 10.0 eq), TEA (0.20 g, 1.9 mmol, 10 eq) and EtOH (4.0 mL) at room temperature. The reaction was stirred at 80° C. for 8 h. The mixture was allowed to cool to room temperature, then concentrated under reduced pressure. The residue was dissolved in DCM (3.0 mL) and purified by silica gel column chromatography (40:60 Petroleum ether/THF) to afford 7-((3aS,4R,6R,6aR)-2,2-dimethyl-6-(3-(1-(tetrahydro-2H-pyran-2-yl)-1H-pyrazol-4-yl)phenyl)tetrahydro-4H-cyclopenta[d][1,3]dioxol-4-yl)-N-(4-methoxybenzyl)-7H-pyrrolo[2,3-d]pyrimidin-4-amine 45d (90 mg, 75%) as a yellow oil. LCMS (conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-2.5 min, 1.50 L/min): T_{R}=1.76 min, ES m/z [M+H]⁺: 621.

### (15,2R,3R,5R)-3-(3-(1H-pyrazol-4-yl)phenyl)-5-(4-amino-7H-pyrrolo[2,3-d]pyrimidin-7-yl)cyclopentane-1,2-diol (652)

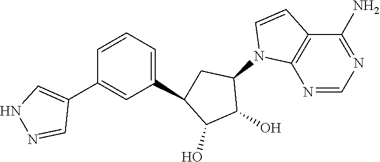 [SMILES: Nc1ncnc2c1ccn2[C@@H]1C[C@H](c2cccc(-c3cn[nH]c3)c2)[C@@H](O)[C@H]1O]

Into a 40 mL vial were added 7-((3aS,4R,6R,6aR)-2,2-dimethyl-6-(3-(1-(tetrahydro-2H-pyran-2-yl)-1H-pyrazol-4-yl)phenyl)tetrahydro-4H-cyclopenta[d][1,3]dioxol-4-yl)-N-(4-methoxybenzyl)-7H-pyrrolo[2,3-d]pyrimidin-4-amine 45d (80 mg, 0.13 mmol, 1.0 eq) and TFA (3.0 mL) at room temperature, The reaction was stirred at 80° C. for 1 h. The mixture was allowed to cool to room temperature, then concentrated under reduced pressure. The residue was dissolved in MeOH (3.0 mL). This mixture was basified to pH>8 with Et₃N and purified by reverse phase flash with the following conditions (Column: Welch Utimate AQ-C18, 50*250 mm*10 μm; Mobile Phase A: Water (10 mmol/L NH₄HCO₃+0.1% NH₃·H₂O), Mobile Phase B: ACN; Flow rate: 80 mL/min; Gradient: 5% B to 60% B in 15 min, 60% B; Wavelength: 220 nm) to afford (1S,2R,3R,5R)-3-(3-(1H-pyrazol-4-yl)phenyl)-5-(4-amino-7H-pyrrolo[2,3-d]pyrimidin-7-yl)cyclopentane-1,2-diol 652 (14.2 mg, 29%) as a white solid. LCMS (Cortecs C18+, 50*3.0 mm, 2.7 μm, Mobile Phase A: Water/0.02% TFA, Mobile Phase B: Acetonitrile, 5%-95% B-3 min, 1.50 L/min): T_{R}=0.76 min, ES m/z [M+H]⁺: 377. 1H NMR (300 MHZ, DMSO-d₆) δ 12.93 (s, 1H), 8.17 (br, s, 1H), 8.08 (s, 1H), 7.95 (br, s, 1H), 7.60 (d, J=1.8 Hz, 1H), 7.50-7.43 (m, 1H), 7.39 (d, J=3.6 Hz, 1H), 7.35-7.26 (m, 1H), 7.25-7.19 (m, 1H), 6.94 (s, 2H), 6.59 (d, J=3.5 Hz, 1H), 4.98-4.85 (m, 3H), 4.38-4.26 (m, 1H), 4.17-4.09 (m, 1H), 3.18-3.06 (m, 1H), 2.46-2.30 (m, 1H), 2.13-1.96 (m, 1H).

### Synthesis of (1R,2S,3R,5R)-3-(4-amino-7H-pyrrolo[2,3-d]pyrimidin-7-yl)-5-(3-(thiazol-5-yl)phenyl)cyclopentane-1,2-diol (653)

### 7-((3aS,4R,6R,6aR)-2,2-dimethyl-6-(3-(thiazol-5-yl)phenyl)tetrahydro-4H-cyclopenta[d][1,3]dioxol-4-yl)-N-(4-methoxybenzyl)-7H-pyrrolo[2,3-d]pyrimidin-4-amine (49d)

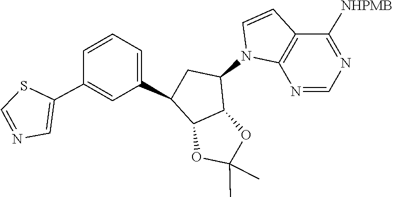

Into a 40 mL vial were added 7-[(3aS,4R,6R,6aR)-6-(3-bromophenyl)-2,2-dimethyl-tetrahydro-3aH-cyclopenta[d][1,3]dioxol-4-yl]-N-[(4-methoxyphenyl)methyl]pyrrolo[2,3-d]pyrimidin-4-amine 48d (80 mg, 0.146 mmol, 1.0 eq), 5-(4,4,5,5-tetramethyl-1,3,2-dioxaborolan-2-yl)-1,3-thiazole (46.1 mg, 0.2 mmol, 1.5 eq), Pd (PPh₃) 4 (16.8 mg, 0.015 mmol, 0.1 eq), Na₂CO₃ (46.3 mg, 0.4 mmol, 3.0 eq), dioxane (3.0 mL) and H₂O (0.3 mL) at room temperature under N₂ atmosphere. The reaction was stirred at 100° C. for 2 h. The mixture was allowed to cool to room temperature. The resulting mixture was extracted with AcOEt (3×20 mL). The combined organic layers were washed with brine and dried over anhydrous Na₂SO₄. After filtration, the filtrate was concentrated under reduced pressure. The residue was dissolved in DCM (3.0 mL) and purified by silica gel column chromatography (50:50 Petroleum ether/THF) to afford 7-((3aS,4R,6R,6aR)-2,2-dimethyl-6-(3-(thiazol-5-yl)phenyl)tetrahydro-4H-cyclopenta[d][1,3]dioxol-4-yl)-N-(4-methoxybenzyl)-7H-pyrrolo[2,3-d]pyrimidin-4-amine 50d (40 mg, 49%) as a white solid. LCMS (conditions L-column3 ODS, 50*3.0 mm, 3.0 μm, Mobile Phase Water/5 mM NH₄HCO₃+0.05% Ammonia, Mobile Phase B: Acetonitrile/5% water, 2%-100% B-3-2.5 min (+), 1.50 L/min): T_{R}=1.76 min, [M+H]⁺: 554.

### (1R,2S,3R,5R)-3-(4-amino-7H-pyrrolo[2,3-d]pyrimidin-7-yl)-5-(3-(thiazol-5-yl)phenyl)cyclopentane-1,2-diol (653)

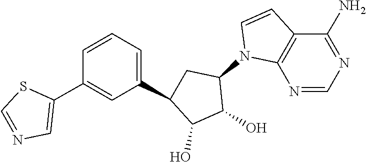 [SMILES: Nc1ncnc2c1ccn2[C@@H]1C[C@H](c2cccc(-c3cncs3)c2)[C@@H](O)[C@H]1O]

Into a 40 mL vial were added 7-((3aS,4R,6R,6aR)-2,2-dimethyl-6-(3-(thiazol-5-yl)phenyl)tetrahydro-4H-cyclopenta[d][1,3]dioxol-4-yl)-N-(4-methoxybenzyl)-7H-pyrrolo[2,3-d]pyrimidin-4-amine 50d (40 mg, 0.081 mmol, 1.0 eq) and TFA (3.0 mL). The reaction was stirred at 80° C. for 1 h. The mixture was allowed to cool to room temperature, then concentrated under reduced pressure. The residue was dissolved in MeOH (3.0 mL) and purified by reverse phase flash with the following conditions (Column: Welch Utimate AQ-C18, 50*250 mm*10 μm; Mobile Phase A: Water (10 mmol/L NH₄HCO₃+0.1% NH₃·H₂O), Mobile Phase B: ACN; Flow rate: 80 mL/min; Gradient: 5% B to 65% B in 15 min, 65% B; Wavelength: 220 nm) to afford (1R,2S,3R,5R)-3-(4-amino-7H-pyrrolo[2,3-d]pyrimidin-7-yl)-5-(3-(thiazol-5-yl)phenyl)cyclopentane-1,2-diol 653 (5.7 mg, 16%) as a white solid. LCMS (Cortecs C18+, 50*3.0 mm, 2.7 μm, 1.8 μm, Mobile Phase A: Water/0.02% TFA, Mobile Phase B: Acetonitrile, 5%-95% B-3 min, 1.50 L/min): T_{R}=0.83 min, ES m/z [M+H]⁺: 394. ¹H NMR (300 MHZ, DMSO-d₆) δ 9.11 (s, 1H), 8.40 (s, 1H), 8.34 (s, 1H), 7.82 (d, J=3.7 Hz, 1H), 7.68 (s, 1H), 7.60-7.51 (m, 1H), 7.49-7.39 (m, 2H), 7.03 (d, J=3.6 Hz, 1H), 5.09-4.95 (m, 1H), 4.31 (t, J=6.9 Hz, 1H), 4.11 (t, J=6.8 Hz, 1H), 3.26-3.11 (m, 1H), 2.50-2.35 (m, 1H), 2.08 (q, J=11.8 Hz, 1H).

### Synthesis of (1R,2S,3R,5R)-3-(4-amino-7H-pyrrolo[2,3-d]pyrimidin-7-yl)-5-(3-(thiazol-4-yl)phenyl)cyclopentane-1,2-diol (654)

### (3aS,45,6R,6aR)-6-(3-bromophenyl)-2,2-dimethyltetrahydro-4H-cyclopenta[d][1,3]dioxol-4-ol (46d)

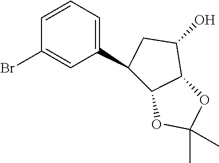 [SMILES: CC1(C)O[C@@H]2[C@H](O1)[C@@H](c1cccc(Br)c1)C[C@@H]2O]

To a stirred solution of (3aR,6R,6aR)-6-(3-bromophenyl)-2,2-dimethyl-tetrahydrocyclopenta[d][1,3]dioxol-4-one 36d (0.45 g, 1.4 mmol, 1.0 eq) in MeOH (5.0 mL) was added NaBH₄ (0.11 g, 2.9 mmol, 2.0 eq) in portions at room temperature over 20 min. The reaction was quenched with H₂O then concentrated under reduced pressure. The residue was dissolved in DCM (5.0 mL) and purified by silica gel column chromatography (70:30 Petroleum ether/THF) to afford (3aS,4S,6R,6aR)-6-(3-bromophenyl)-2,2-dimethyltetrahydro-4H-cyclopenta[d][1,3]dioxol-4-ol 46d (300 mg, 66%) as a yellow oil. LCMS (conditions L-column3 ODS, 50*3.0 mm, 3.0 μm, Mobile Phase A: Water/5 mM NH₄HCO₃+0.05% Ammonia, Mobile Phase B: Acetonitrile/5% water, 2%-100% B-3-2.5 min (+,-), 1.50 L/min): T_{R}=1.55 min, [M−H]: 311.

### 7-((3aS,4R,6R,6aR)-6-(3-bromophenyl)-2,2-dimethyltetrahydro-4H-cyclopenta[d][1,3]dioxol-4-yl)-4-chloro-7H-pyrrolo[2,3-d]pyrimidine (47d)

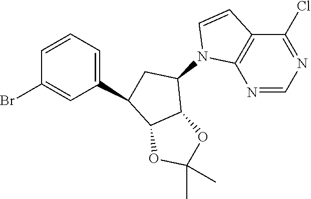 [SMILES: CC1(C)O[C@@H]2[C@H](O1)[C@@H](c1cccc(Br)c1)C[C@H]2n1ccc2c(Cl)ncnc21]

To a stirred solution of (3aS,4S,6R,6aR)-6-(3-bromophenyl)-2,2-dimethyl-tetrahydro-3aH-cyclopenta[d][1,3]dioxol-4-ol (0.3 g, 0.9 mmol, 1.2 eq), 4-chloro-7H-pyrrolo[2,3-d]pyrimidine 46d (0.13 g, 0.8 mmol, 1.0 eq) and PPh₃ (0.43 g, 1.6 mmol, 2.0 eq) in toluene (10.0 mL) was added DBAD (0.37 g, 1.6 mmol, 2.0 equiv) in toluene (3.0 mL) dropwise at 0° C. under N₂ atmosphere. The reaction was stirred at 50° C. for 2 h. The mixture was allowed to cool to room temperature, then concentrated under reduced pressure. The residue was dissolved in DCM (5.0 mL) and purified by silica gel column chromatography (60:40 Petroleum ether/THF) to afford 7-((3aS,4R,6R,6aR)-6-(3-bromophenyl)-2,2-dimethyltetrahydro-4H-cyclopenta[d][1,3]dioxol-4-yl)-4-chloro-7H-pyrrolo[2,3-d]pyrimidine 47d (300 mg, 82%) as a yellow oil. LCMS (conditions Proshell HPH-C18, 50*3.0 mm, 2.7 μm, Mobile Phase A: Water (0.01% FA), Mobile Phase B: Acetonitrile/0.01% FA, 2%-100% B-2.5 min, 1.50 L/min): T_{R}=1.11 min, [M+H]⁺: 448, 450.

### 7-((3aS,4R,6R,6aR)-6-(3-bromophenyl)-2,2-dimethyltetrahydro-4H-cyclopenta[d][1,3]dioxol-4-yl)-N-(4-methoxybenzyl)-7H-pyrrolo[2,3-d]pyrimidin-4-amine (48d)

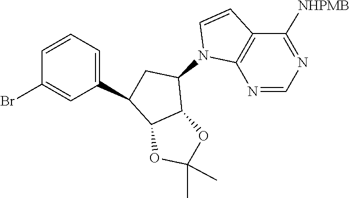

Into a 40 mL vial were added 7-((3aS,4R,6R,6aR)-6-(3-bromophenyl)-2,2-dimethyltetrahydro-4H-cyclopenta[d][1,3]dioxol-4-yl)-4-chloro-7H-pyrrolo[2,3-d]pyrimidine 47d (0.28 g, 0.6 mmol, 1.0 eq), 4-methoxy-benzenemethanamine (0.26 g, 1.9 mmol, 3.0 eq), TEA (0.19 g, 1.9 mmol, 3.0 eq) and EtOH (5.0 mL) at room temperature, The reaction was stirred at 80° C. for 1 h. The mixture was allowed to cool to room temperature, then concentrated under reduced pressure. The residue was dissolved in DCM (4.0 mL) and purified by silica gel column chromatography (45:55 Petroleum ether/THF) to afford 7-((3aS,4R,6R,6aR)-6-(3-bromophenyl)-2,2-dimethyltetrahydro-4H-cyclopenta[d][1,3]dioxol-4-yl)-N-(4-methoxybenzyl)-7H-pyrrolo[2,3-d]pyrimidin-4-amine 48d (250 mg, 73%) as a yellow solid. LCMS (conditions L-column3 ODS, 50*3.0 mm, 3.0 μm, Mobile Phase A: Water/5 mM NH₄HCO₃+0.05% Ammonia, Mobile Phase B: Acetonitrile/5% water, 2%-100% B-3-2.5 min (+), 1.50 L/min): T_{R}=1.86 min, [M+H]⁺: 549, 561.

### 7-((3aS,4R,6R,6aR)-2,2-dimethyl-6-(3-(thiazol-4-yl)phenyl)tetrahydro-4H-cyclopenta[d][1,3]dioxol-4-yl)-N-(4-methoxybenzyl)-7H-pyrrolo[2,3-d]pyrimidin-4-amine (49d)

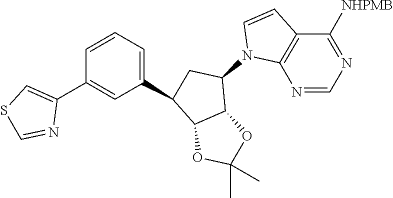

Into a 40 mL vial were added 7-[(3aS,4R,6R,6aR)-6-(3-bromophenyl)-2,2-dimethyl-tetrahydro-3aH-cyclopenta[d][1,3]dioxol-4-yl]-N-[(4-methoxyphenyl)methyl]pyrrolo[2,3-d]pyrimidin-4-amine 48d (0.10 g, 0.2 mmol, 1.0 eq), 4-(tributylstannyl)-1,3-thiazole (0.14 g, 0.4 mmol, 2.0 eq), Pd (PPh₃) 2Cl₂ (12.77 mg, 0.018 mmol, 0.1 eq) and toluene (3.0 mL) at room temperature under N₂ atmosphere. The reaction was stirred at 120° C. for 2 h. The mixture was allowed to cool to room temperature. The resulting mixture was extracted with AcOEt (3×10.0 mL). The combined organic layers were washed with brine and dried over anhydrous Na₂SO₄. After filtration, the filtrate was concentrated under reduced pressure. The residue was dissolved in DCM (3.0 mL) and purified by silica gel column chromatography (50:50 Petroleum ether/THF) to afford 7-[(3aS,4R,6R,6aR)-2,2-dimethyl-6-[3-(1,3-thiazol-4-yl)phenyl]-tetrahydro-3aH-cyclopenta[d][1,3]dioxol-4-yl]-N-[(4-methoxyphenyl)methyl]pyrrolo[2,3-d]pyrimidin-4-amine 49d (80 mg, 79%) as white oil. LCMS (conditions L-column3 ODS, 50*3.0 mm, 3.0 μm, Mobile Phase A: Water/5 mM NH₄HCO₃+0.05% Ammonia, Mobile Phase B: Acetonitrile/5% water, 2%-100% B-2.5 min (+), 1.50 L/min): T_{R}=1.73 min, ES m/z [M+H]⁺: 554.

### (1R,2S,3R,5R)-3-{4-aminopyrrolo[2,3-d]pyrimidin-7-yl}-5-[3-(1,3-thiazol-4-yl)phenyl]cyclopentane-1,2-diol (654)

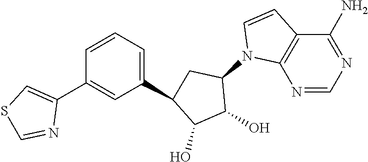 [SMILES: Nc1ncnc2c1ccn2[C@@H]1C[C@H](c2cccc(-c3cscn3)c2)[C@@H](O)[C@H]1O]

Into a 40 mL vial were added 7-[(3aS,4R,6R,6aR)-2,2-dimethyl-6-[3-(1,3-thiazol-4-yl)phenyl]-tetrahydro-3aH-cyclopenta[d][1,3]dioxol-4-yl]-N-[(4-methoxyphenyl)methyl]pyrrolo[2,3-d]pyrimidin-4-amine 49d (75 mg, 0.135 mmol, 1.0 eq) and TFA (3.0 mL). The reaction was stirred at 80° C. for 1 h. The mixture was allowed to cool to room temperature, then concentrated under reduced pressure. The residue was dissolved in MeOH (3.0 mL) and purified by reverse phase flash with the following conditions (Column: Welch Utimate AQ-C18, 50*250 mm*10 μm; Mobile Phase A: Water (10 mmol/L NH₄HCO₃+0.1% NH₃·H₂O), Mobile Phase B: ACN; Flow rate: 80 mL/min; Gradient: 5% B to 50% B in 15 min, 50% B; Wavelength: 220 nm) to afford (1R,2S,3R,5R)-3-(4-amino-7H-pyrrolo[2,3-d]pyrimidin-7-yl)-5-(3-(thiazol-4-yl)phenyl)cyclopentane-1,2-diol 654 (25.8 mg, 47%) as a white solid. LCMS (Cortecs C18+, 50*3.0 mm, 2.7 μm, Mobile Phase A: Water/0.02% TFA, Mobile Phase B: Acetonitrile, 5%-95% B-3 min, 1.50 L/min): T_{R}=0.84 min, ES m/z [M+H]⁺: 394. ¹H NMR (300 MHZ, DMSO-d₆) δ9.21 (d, J=1.9 Hz, 1H), 8.18 (d, J=1.9 Hz, 1H), 8.08 (s, 1H), 8.02 (s, 1H), 7.88-7.83 (m, 1H), 7.44-7.37 (m, 3H), 6.94 (s, 2H), 6.59 (d, J=3.5 Hz, 1H), 5.06-4.79 (m, 3H), 4.33 (q, J=6.3 Hz, 1H), 4.16 (q, J=6.7 Hz, 1H), 3.25-3.11 (m, 1H), 2.48-2.32 (m, 1H), 2.09 (q, J=11.8 Hz, 1H).

### Synthesis of (1R,2S,3R,5R)-3-((2-chloro-6-methylpyrimidin-4-yl)(methyl)amino)-5-(4-hydroxyphenyl)cyclopentane-1,2-diol (655)

### (3aR,6R,6aR)-6-(4-((Tert-butyldimethylsilyl)oxy)phenyl)-2,2-dimethyltetrahydro-4H-cyclopenta[d][1,3]dioxol-4-one (81d)

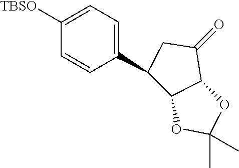 [SMILES: CC1(C)O[C@@H]2[C@@H](c3ccc(O[Si](C)(C)C(C)(C)C)cc3)CC(=O)[C@@H]2O1]

Into a 40 mL vial were added (3aR,6aR)-2,2-dimethyl-3aH,6aH-cyclopenta[d][1,3]dioxol-4-one (0.50 g, 3.2 mmol, 1.0 eq), 4-[(tert-butyldimethylsilyl)oxy]phenylboronic acid (1.23 g, 4.8 mmol, 1.5 eq), cyclooctadiene rhodium chloride dimer (0.24 g, 0.5 mmol, 0.15 eq), (R)-BINAP (0.30 g, 0.5 mmol, 0.15 eq) and H₂O (0.5 mL). The reaction was stirred at 100° C. for 3 h. The mixture was allowed to cool down to temperature, then was extracted with AcOEt (3×20.0 mL). The combined organic layers were washed with brine and dried over anhydrous Na₂SO₄. After filtration, the filtrate was concentrated under reduced pressure. The residue was dissolved in DCM (3.0 mL) and purified by silica gel column chromatography (20:80 Petroleum ether/THF) to afford (3aR,6R,6aR)-6-(4-((tert-butyldimethylsilyl)oxy)phenyl)-2,2-dimethyltetrahydro-4H-cyclopenta[d][1,3]dioxol-4-one 81d (250 mg, 21%) as a yellow oil. ¹H NMR (400 MHZ, Chloroform-d) 87.04-6.96 (m, 2H), 6.84-6.77 (m, 2H), 4.71 (d, J=5.3 Hz, 1H), 4.36 (d, J=5.4 Hz, 1H), 3.67-3.57 (m, 1H), 2.57-2.43 (m, 1H), 2.06 (s, 1H), 1.53 (s, 3H), 1.36 (s, 3H), 0.99 (s, 9H), 0.20 (s, 6H).

### (3aS,45,6R,6aR)-6-(4-((Tert-butyldimethylsilyl)oxy)phenyl)-2,2-dimethyltetrahydro-4H-cyclopenta[d][1,3]dioxol-4-ol (82d)

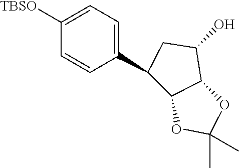 [SMILES: CC1(C)O[C@@H]2[C@H](O1)[C@@H](c1ccc(O[Si](C)(C)C(C)(C)C)cc1)C[C@@H]2O]

To a stirred solution of (3aR,6R,6aR)-6-(4-((tert-butyldimethylsilyl)oxy)phenyl)-2,2-dimethyltetrahydro-4H-cyclopenta[d][1,3]dioxol-4-one 81d (0.21 g, 0.579 mmol, 1 eq) in MeOH (3.0 mL) was added NaBH₄ (44.0 mg, 1.158 mmol, 2.0 eq) in portions at 0° C. The reaction was stirred at room temperature for 20 min, then quenched with H₂O. The resulting mixture was concentrated under reduced pressure. The residue was dissolved in DCM (3.0 mL) and purified by silica gel column chromatography (60:40 Petroleum ether/THF) to afford (3aS,4S,6R,6aR)-6-(4-((tert-butyldimethylsilyl)oxy)phenyl)-2,2-dimethyltetrahydro-4H-cyclopenta[d][1,3]dioxol-4-ol 82d (200 mg, 94%) as a yellow oil. LCMS (conditions L-column3 ODS, 50*3.0 mm, 3.0 μm, Mobile Phase Water/5 mM NH₄HCO₃+0.05% Ammonia, Mobile Phase B: Acetonitrile/5% water, 2%-100% B-3 min, 1.50 L/min): T_{R}=1.93 min, ES m/z [M−H]: 363.

### 7-((3aS,4R,6R,6aR)-6-(4-((Tert-butyldimethylsilyl)oxy)phenyl)-2,2-dimethyltetrahydro-4H-cyclopenta[d][1,3]dioxol-4-yl)-2,4-dichloro-7H-pyrrolo[2,3-d]pyrimidine (83d)

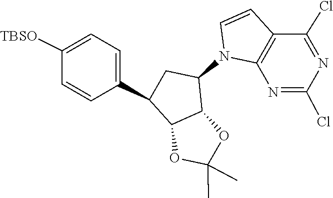 [SMILES: CC1(C)O[C@@H]2[C@H](O1)[C@@H](c1ccc(O[Si](C)(C)C(C)(C)C)cc1)C[C@H]2n1ccc2c(Cl)nc(Cl)nc21]

To a stirred solution of 2,4-dichloro-7H-pyrrolo[2,3-d]pyrimidine (85 mg, 0.45 mmol, 1.0 eq), (3aS,4S,6R,6aR)-6-(4-((tert-butyldimethylsilyl)oxy)phenyl)-2,2-dimethyltetrahydro-4H-cyclopenta[d][1,3]dioxol-4-ol 82d (0.20 g, 0.5 mmol, 1.2 eq) and PPh₃ (0.23 g, 0.9 mmol, 2.0 eq) in THF (2.0 mL) was added DIAD (0.18 g, 0.9 mmol, 2 eq) in THF (2.0 mL) dropwise at 0° C. under N₂ atmosphere. The reaction was stirred at room temperature for 12 h. The resulting mixture was concentrated under reduced pressure. The residue was dissolved in DCM (4.0 mL) and purified by silica gel column chromatography (60:40 Petroleum ether/THF) to afford 7-((3aS,4R,6R,6aR)-6-(4-((tert-butyldimethylsilyl)oxy)phenyl)-2,2-dimethyltetrahydro-4H-cyclopenta[d][1,3]dioxol-4-yl)-2,4-dichloro-7H-pyrrolo[2,3-d]pyrimidine 83d (160 mg, 66%) as a white solid. LCMS (conditions L-column3 ODS, 50*3.0 mm, 3.0 μm, Mobile Phase A: Water/5 mM NH₄HCO₃+0.05% Ammonia, Mobile Phase B: Acetonitrile/5% water, 2%-100% B-2.5 min, 1.50 L/min): T_{R}=1.78 min, ES m/z [M+H]⁺: 534, 536.

### 7-((3aS,4R,6R,6aR)-6-(4-((Tert-butyldimethylsilyl)oxy)phenyl)-2,2-dimethyltetrahydro-4H-cyclopenta[d][1,3]dioxol-4-yl)-2-chloro-N-(4-methoxybenzyl)-7H-pyrrolo[2,3-d]pyrimidin-4-amine (84d)

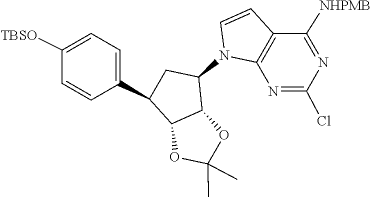

Into a 40 mL vial were added 7-((3aS,4R,6R,6aR)-6-(4-((tert-butyldimethylsilyl)oxy)phenyl)-2,2-dimethyltetrahydro-4H-cyclopenta[d][1,3]dioxol-4-yl)-2,4-dichloro-7H-pyrrolo[2,3-d]pyrimidine 83d (90.0 mg, 0.168 mmol, 1.0 eq), 4-methoxy-benzenemethanamine (70.0 mg, 0.5 mmol, 3.0 eq), Et₃N (51.1 mg, 0.5 mmol, 3.0 eq) and EtOH (2.0 mL). The reaction was stirred at 80° C. for 2 h. The mixture was allowed to cool to room temperature, then concentrated under reduced pressure. The residue was dissolved in DCM (3.0 mL) and purified by silica gel column chromatography (50:50 Petroleum ether/THF) to afford 7-((3aS,4R,6R,6aR)-6-(4-((tert-butyldimethylsilyl)oxy)phenyl)-2,2-dimethyltetrahydro-4H-cyclopenta[d][1,3]dioxol-4-yl)-2-chloro-N-(4-methoxybenzyl)-7H-pyrrolo[2,3-d]pyrimidin-4-amine 84d (80 mg, 75%) as a yellow oil. LCMS (conditions L-column3 ODS, 50*3.0 mm, 3.0 μm, Mobile Phase A: Water/5 mM NH₄HCO₃+0.05% Ammonia, Mobile Phase B: Acetonitrile/5% water, 2%-100% B-3 min, 1.50 L/min): T_{R}=2.67 min, ES m/z [M+H]⁺: 635, 637.

### (1R,2S,3R,5R)-3-((2-chloro-6-methylpyrimidin-4-yl)(methyl)amino)-5-(4-hydroxyphenyl)cyclopentane-1,2-diol (655)

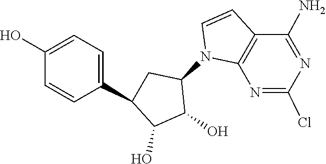 [SMILES: Nc1nc(Cl)nc2c1ccn2[C@@H]1C[C@H](c2ccc(O)cc2)[C@@H](O)[C@H]1O]

Into a 40 mL vial were added 7-((3aS,4R,6R,6aR)-6-(4-((tert-butyldimethylsilyl)oxy)phenyl)-2,2-dimethyltetrahydro-4H-cyclopenta[d][1,3]dioxol-4-yl)-2-chloro-N-(4-methoxybenzyl)-7H-pyrrolo[2,3-d]pyrimidin-4-amine 84d (80 mg, 0.13 mmol, 1.0 eq) and TFA (3.0 mL). The reaction was stirred at 80° C. for 20 min. The mixture was allowed to cool to room temperature, then concentrated under reduced pressure. The residue was dissolved in CH₃CN (3.0 mL), basified to pH>8 with Et₃N and purified by reverse phase flash chromatography with the following conditions (Column: Welch Ultimate XB-C18, 50*250 cm, 10 μm; Phase A: Water (0.05% NH₃·H₂O), Mobile Phase B: ACN; Flow rate: 80 mL/min; Gradient: 5% B-5% B-2 min, 5% B-50% B-12 min, 50% B) to afford (1R,2S,3R,5R)-3-((2-chloro-6-methylpyrimidin-4-yl)(methyl)amino)-5-(4-hydroxyphenyl)cyclopentane-1,2-diol 655 (11.9 mg, 30%) as a white solid. LCMS (Cortecs C18+, 50*3.0 mm, 2.7 μm, Mobile Phase A: Water/0.02% TFA, Mobile Phase B: Acetonitrile, 5%-95% B-3 min, 1.50 L/min): T_{R}=0.89 min, ES m/z [M+H]⁺: 361, 363. ¹H NMR (300 MHZ, DMSO-d₆) δ9.18 (br, s, 1H), 7.45 (br, s, 2H), 7.38 (d, J=3.6 Hz, 1H), 7.26-7.13 (m, 2H), 6.77-6.69 (m, 2H), 6.59 (d, J=3.5 Hz, 1H), 4.89 (d, J=6.0 Hz, 1H), 4.86-4.72 (m, 2H), 4.24 (q, J=6.4 Hz, 1H), 3.94 (q, J=6.2 Hz, 1H), 3.08-2.83 (m, 1H), 2.37-2.23 (m, 1H), 2.03-1.79 (m, 1H).

### Synthesis of (1R,2S,3R,5R)-3-(2-chloro-4-(methylamino)-7H-pyrrolo[2,3-d]pyrimidin-7-yl)-5-(4-hydroxyphenyl)cyclopentane-1,2-diol (656)

### 7-((3aS,4R,6R,6aR)-6-(4-((Tert-butyldimethylsilyl)oxy)phenyl)-2,2-dimethyltetrahydro-4H-cyclopenta[d][1,3]dioxol-4-yl)-2-chloro-N-(4-methoxybenzyl)-N-methyl-7H-pyrrolo[2,3-d]pyrimidin-4-amine (85d)

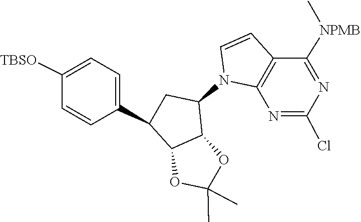

Into a 40 mL vial were added 7-[(3aS,4R,6R,6aR)-6-{4-[(tert-butyldimethylsilyl)oxy]phenyl}-2,2-dimethyl-tetrahydro-3aH-cyclopenta[d][1,3]dioxol-4-yl]-2,4-dichloropyrrolo[2,3-d]pyrimidine 83d (50 mg, 0.094 mmol, 1.0 eq), 1-(4-methoxyphenyl)-N-methylmethanamine (70.7 mg, 0.5 mmol, 5.0 eq), Et₃N (30.0 mg, 0.3 mmol, 3.0 eq) and EtOH (2.0 mL). The reaction was stirred at 80° C. for 2 h. The mixture was allowed to cool to room temperature, then concentrated under reduced pressure. The residue was dissolved in DCM (2.0 mL) and purified by Prep-TLC (50:50 Petroleum ether/THF) to afford 7-[(3aS,4R,6R,6aR)-6-{4-[(tert-butyldimethylsilyl)oxy]phenyl}-2,2-dimethyl-tetrahydro-3aH-cyclopenta[d][1,3]dioxol-4-yl]-2-chloro-N-[(4-methoxyphenyl)methyl]-N-methylpyrrolo[2,3-d]pyrimidin-4-amine 85d (40 mg, 65%) as a yellow oil. LCMS (conditions L-column3 ODS, 50*3.0 mm, 3.0 μm, Mobile Phase Water/5 mM NH₄HCO₃+0.05% Ammonia, Mobile Phase B: Acetonitrile/5% water, 2%-100% B-2.5 min, 1.50 L/min): T_{R}=2.40 min, ES m/z [M+H]⁺: 649, 651.

### (1R,2S,3R,5R)-3-(2-chloro-4-(methylamino)-7H-pyrrolo[2,3-d]pyrimidin-7-yl)-5-(4-hydroxyphenyl)cyclopentane-1,2-diol (656)

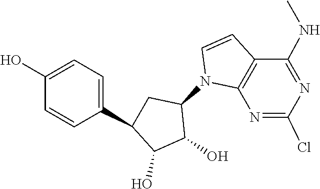 [SMILES: CNc1nc(Cl)nc2c1ccn2[C@@H]1C[C@H](c2ccc(O)cc2)[C@@H](O)[C@H]1O]

Into a 40 mL vial were added 7-((3aS,4R,6R,6aR)-6-(4-((tert-butyldimethylsilyl)oxy)phenyl)-2,2-dimethyltetrahydro-4H-cyclopenta[d][1,3]dioxol-4-yl)-2-chloro-N-(4-methoxybenzyl)-N-methyl-7H-pyrrolo[2,3-d]pyrimidin-4-amine 85d (40 mg, 0.062 mmol, 1.0 eq) and TFA (3.0 mL). The reaction was stirred at 80° C. for 1 h. The mixture was allowed to cool to room temperature, then concentrated under reduced pressure. The residue was dissolved in CH₃CN (3.0 mL). The mixture was basified to pH>8 with Et₃N and purified by reverse phase flash with the following conditions (Column: Welch Ultimate XB-C18, 50*250 cm, 10 μm; Phase A: Water (0.05% NH₃·H₂O), Mobile Phase B: ACN; Flow rate: 80 mL/min; Gradient: 5% B-5% B-2 min, 5% B-50% B-12 mim,50% B; Wavelength: 254 nm) to afford (1R,2S,3R,5R)-3-[2-chloro-4-(methylamino)pyrrolo[2,3-d]pyrimidin-7-yl]-5-(4-hydroxyphenyl)cyclopentane-1,2-diol 656 (6.6 mg, 30%) as a white solid. LCMS (Cortecs C18+, 50*3.0 mm, 2.7 μm, Mobile Phase A: Water/0.02% TFA, Mobile Phase B: Acetonitrile, 5%-95% B-3 min, 1.50 L/min): T_{R}=1.04 min, ES m/z [M+H]⁺: 375, 377. ¹H NMR (300 MHz, DMSO-d₆) δ9.18 (s, 1H), 7.90 (br, s, 1H), 7.37 (d, J=3.5 Hz, 1H), 7.20 (d, J=8.1 Hz, 2H), 6.72 (d, J=8.1 Hz, 2H), 6.59 (d, J=3.5 Hz, 1H), 4.93-4.74 (m, 3H), 4.24 (q, J=6.5 Hz, 1H), 3.95 (q, j=6.2 Hz, 1H), 3.05-2.93 (m, 4H), 2.35-2.26 (m, 1H), 1.99-1.83 (m, 1H).

### Synthesis of (1R,2S,3R,5R)-3-[4-(methylamino)pyrrolo[2,3-d]pyrimidin-7-yl]-5-(1H-pyrazol-4-yl)cyclopentane-1,2-diol (657)

### 7-[(3aS,4R,6R,6aR)-2,2-Dimethyl-6-[1-(oxan-2-yl)pyrazol-4-yl]-tetrahydro-3aH-cyclopenta[d][1,3]dioxol-4-yl]-N-methylpyrrolo[2,3-d]pyrimidin-4-amine (152d)

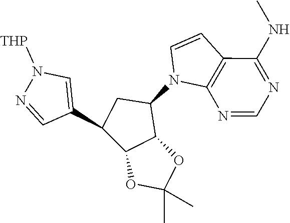

Into an 8 mL vial were added 4-[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]-1-(oxan-2-yl)pyrazole (96 mg, 0.22 mmol, 1.0 eq) and 2M methylamine in methanol (2 mL) at room temperature. The resulting mixture was stirred for 1 h at 80° C. The resulting mixture was concentrated to afford 7-[(3aS,4R,6R,6aR)-2,2-dimethyl-6-[1-(oxan-2-yl)pyrazol-4-yl]-tetrahydro-3aH-cyclopenta[d][1,3]dioxol-4-yl]-N-methylpyrrolo[2,3-d]pyrimidin-4-amine 152d (80 mg, 84%) as a light yellow solid. LCMS (conditions XBridge C18, 50*3.0 mm, 3.5 μm, Mobile Phase A: Water/5 mM NH₄HCO₃, Mobile Phase B: Acetonitrile, 5%-100% B-2 min, 1.50 mL/min) T_{R}=1.25 min; ES m/z [M+H]⁺: 439.

### (1R,2S,3R,5R)-3-[4-(Methylamino)pyrrolo[2,3-d]pyrimidin-7-yl]-5-(1H-pyrazol-4-yl)cyclopentane-1,2-diol (657)

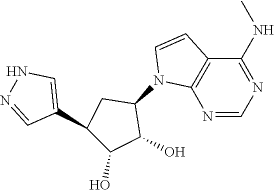 [SMILES: CNc1ncnc2c1ccn2[C@@H]1C[C@H](c2cn[nH]c2)[C@@H](O)[C@H]1O]

Into an 8 mL vial were added 7-[(3aS,4R,6R,6aR)-2,2-dimethyl-6-[1-(oxan-2-yl)pyrazol-4-yl]-tetrahydro-3aH-cyclopenta[d][1,3]dioxol-4-yl]-N-methylpyrrolo[2,3-d]pyrimidin-4-amine 152d (80 mg, 0.18 mmol, 1.0 eq) and 4M HCl in MeOH (2 mL) at room temperature. The reaction was stirred for 0.5 h at 50° C., then concentrated. The residue was dissolved in MeOH (3 mL) and basified to pH>8 with NH₃·H₂O. The mixture was purified by Prep-HPLC [column, Xbridge Prep C18 OBD column, 5 μm, 19*150 mm; mobile phase, Water (0.03% NH₄OH) and CH₃CN (20-50% over 10 min); Detector, UV 220&254 nm]to afford (1R,2S,3R,5R)-3-[4-(methylamino)pyrrolo[2,3-d]pyrimidin-7-yl]-5-(1H-pyrazol-4-yl)cyclopentane-1,2-diol 657 (25.1 mg, 44%) as a white solid. LCMS (conditions: ZORBAX SB-Aq, 50*4.6 mm, 1.8 μm, Mobile Phase A: Water/0.02% TFA, Mobile Phase B: Acetonitrile, 5%-95% B-3 min, 1.50 mL/min): T_{R}=0.98 min; ES m/z [M+H]⁺: 315. ¹H NMR (300 MHZ, DMSO-d₆) δ 12.55 (br, s, 1H), 8.14 (s, 1H), 7.55 (br, s, 2H), 7.39 (d, J=5.0 Hz, 1H), 7.26 (d, J=3.5 Hz, 1H), 6.55 (d, J=3.5 Hz, 1H), 4.96-4.79 (m, 3H), 4.24 (q, J=6.1 Hz, 1H), 3.93 (q, J=6.2 Hz, 1H), 3.09-3.00 (m, 1H), 2.96 (d, J=4.6 Hz, 3H), 2.48-2.34 (m, 1H), 1.98-1.80 (m, 1H).

### Synthesis of (1R,2S,3R,5R)-3-{4-aminopyrrolo[2,3-d]pyrimidin-7-yl}-5-(1H-pyrazol-4-yl)cyclopentane-1,2-diol (658)

### 4-[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]-1-(oxan-2-yl)pyrazole (150d)

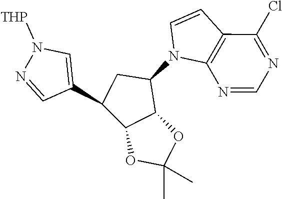

Into an 8 mL vial were added (3aS,4S,6R,6aR)-2,2-dimethyl-6-[1-(oxan-2-yl)pyrazol-4-yl]-tetrahydro-3aH-cyclopenta[d][1,3]dioxol-4-ol (0.2 g, 0.65 mmol, 1.2 eq), 4-chloro-7H-pyrrolo[2,3-d]pyrimidine (83.3 mg, 0.54 mmol, 1.0 eq) and DIAD (0.22 g, 1.08 mmol, 2.0 eq), followed by PPh₃ (0.28 g, 1.08 mmol, 2.0 eq) in THF (5 mL) dropwise at 0° C. The reaction was stirred for 12 h at room temperature, then concentrated. The residue was dissolved in DCM (3 mL) and was purified by silica gel column chromatography (1:1 Petroleum ether: AcOEt) to afford 4-[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]-1-(oxan-2-yl)pyrazole 150d (0.19 g, 80%) as a white solid. LCMS (conditions XBridge C18, 50*3.0 mm, 3.5 μm, Mobile Phase A: Water/5 mM NH₄HCO₃, Mobile Phase B: Acetonitrile, 5%-100% B-2 min, 1.50 mL/min,): T_{R}=1.47 min; ES m/z [M+H]⁺: 444, 446.

### 7-[(3aS,4R,6R,6aR)-2,2-Dimethyl-6-[1-(oxan-2-yl)pyrazol-4-yl]-tetrahydro-3aH-cyclopenta[d][1,3]dioxol-4-yl]-N-[(4-methoxyphenyl)methyl]pyrrolo[2,3-d]pyrimidin-4-amine (151d)

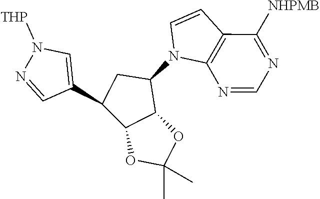

Into an 8 mL vial were added 4-[(3aR,4R,6R,6aS)-6-{4-chloropyrrolo[2,3-d]pyrimidin-7-yl}-2,2-dimethyl-tetrahydro-3aH-cyclopenta[d][1,3]dioxol-4-yl]-1-(oxan-2-yl)pyrazole 150d (96 mg, 0.22 mmol, 1.0 eq), (4-methoxyphenyl)methanamine (89 mg, 0.65 mmol, 3.0 eq) and TEA (43.8 mg, 0.43 mmol, 2.0 eq) in EtOH (2 mL) at room temperature. The reaction was stirred for 1 h at 80° C. The resulting mixture was concentrated to afford 7-[(3aS,4R,6R,6aR)-2,2-dimethyl-6-[1-(oxan-2-yl)pyrazol-4-yl]-tetrahydro-3aH-cyclopenta[d][1,3]dioxol-4-yl]-N-[(4-methoxyphenyl)methyl]pyrrolo[2,3-d]pyrimidin-4-amine 151d (80 mg, 68%) as a yellow solid. LCMS (conditions Xbridge Shield C18, 50*3.0 mm, 3.5 μm, Mobile Phase A: Water/0.05% Ammonia, Mobile Phase B: Acetonitrile, 5%-100% B-3 min, 1.50 mL/min): T_{R}=1.90 min; ES m/z [M+H]⁺: 545.

### (1R,2S,3R,5R)-3-{4-Aminopyrrolo[2,3-d]pyrimidin-7-yl}-5-(1H-pyrazol-4-yl)cyclopentane-1,2-diol (658)

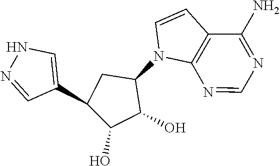 [SMILES: Nc1ncnc2c1ccn2[C@@H]1C[C@H](c2cn[nH]c2)[C@@H](O)[C@H]1O]

Into an 8 mL vial were added 7-[(3aS,4R,6R,6aR)-2,2-dimethyl-6-[1-(oxan-2-yl)pyrazol-4-yl]-tetrahydro-3aH-cyclopenta[d][1,3]dioxol-4-yl]-N-[(4-methoxyphenyl)methyl]pyrrolo[2,3-d]pyrimidin-4-amine 151d (80 mg, 0.15 mmol, 1.0 eq) and TFA (2 mL) at room temperature. The resulting mixture was stirred for 0.5 h at 80° C., then concentrated. The residue was dissolved in MeOH (3 mL) and basified to pH>8 with NH₃·H₂O. The mixture was purified by Prep-HPLC [column, Xbridge Prep C18 OBD column, 5 μm, 19*150 mm; mobile phase, Water (0.03% NH₄OH) and CH₃CN (20-55% CH₃CN over 10 min); Detector, UV 220&254 nm]to afford (1R,2S,3R,5R)-3-{4-aminopyrrolo[2,3-d]pyrimidin-7-yl}-5-(1H-pyrazol-4-yl)cyclopentane-1,2-diol 658 (13.9 mg, 32%) as a white solid. LCMS (conditions: ZORBAX SB-Aq, 50*4.6 mm, 1.8 μm, Mobile Phase A: Water/0.02% TFA, Mobile Phase B: Acetonitrile, 5%-95% B-3 min, 1.50 mL/min): T_{R}=0.94 min; ES m/z [M+H]⁺: 301.1H NMR (400 MHZ, DMSO-d₆) δ 8.05 (s, 1H), 7.55 (s, 2H), 7.27 (d, J=3.5 Hz, 1H), 6.92 (s, 2H), 6.56 (d, J=3.5 Hz, 1H), 4.94-4.80 (m, 3H), 4.25-4.20 (m, 1H), 3.97-3.92 (m, 1H), 3.05-3.01 (m, 1H), 2.46-2.35 (m, 1H), 1.89 (q, J=10.9 Hz, 1H).

### Synthesis of (1R,2S,3R,5R)-3-(4-amino-7H-pyrrolo[2,3-d]pyrimidin-7-yl)-5-(3-(aminomethyl)phenyl)cyclopentane-1,2-diol (659)

### Tert-butyl (3-((3aR,4R,6aR)-2,2-dimethyl-6-oxotetrahydro-4H-cyclopenta[d][1,3]dioxol-4-yl)benzyl)carbamate (122d)

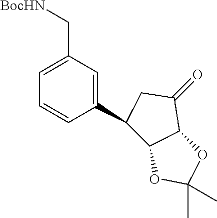 [SMILES: CC(C)(C)OC(=O)NCc1cccc([C@H]2CC(=O)[C@@H]3OC(C)(C)O[C@H]23)c1]

Into a 40 mL vial were added (3aR,6aR)-2,2-dimethyl-3aH, 6aH-cyclopenta[d][1,3]dioxol-4-one (0.5 g, 3.2 mmol, 1.0 eq), 3-{[(tert-butoxycarbonyl)amino]methyl}phenylboronic acid (1.6 g, 6.5 mmol, 2.0 eq), acetylacetonato bis(ethylene) rhodium (I)(84.0 mg, 0.3 mmol, 0.1 eq), (R)-BINAP (0.2 g, 0.3 mmol, 0.1 eq), dioxane (5.0 mL) and H₂O (0.5 mL) at room temperature under N₂ atmosphere. The reaction mixture was stirred at 100° C. for 2 h, then allowed to cool to room temperature. The resulting mixture was concentrated under reduced pressure. The residue was extracted with AcOEt (3×20.0 mL). The combined organic layers were washed with brine and dried over anhydrous Na₂SO₄. After filtration, the filtrate was concentrated under reduced pressure. The residue was dissolved in DCM (3.0 mL) and purified by silica gel column chromatography (70:30 Petroleum ether/THF) to afford tert-butyl (3-((3aR,4R,6aR)-2,2-dimethyl-6-oxotetrahydro-4H-cyclopenta[d][1,3]dioxol-4-yl)benzyl)carbamate 122d (350 mg, 29%) as yellow oil. LCMS (conditions: L-column3 ODS, 50*3.0 mm, 3.0 μm, Mobile Phase A: Water/5 mM NH₄HCO₃+0.05% Ammonia, Mobile Phase B: Acetonitrile, 5%-100% B-2 min, 1.50 L/min): T_{R}=1.45 min; ES m/z [M−H]: 360.

### Tert-butyl (3-((3aR,4R,6S,6aS)-6-hydroxy-2,2-dimethyltetrahydro-4H-cyclopenta[d][1,3]dioxol-4-yl)benzyl)carbamate (123d)

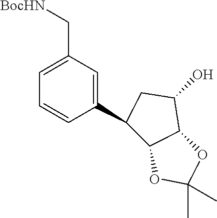 [SMILES: CC(C)(C)OC(=O)NCc1cccc([C@H]2C[C@H](O)[C@@H]3OC(C)(C)O[C@@H]32)c1]

To a stirred solution of tert-butyl (3-((3aR,4R,6aR)-2,2-dimethyl-6-oxotetrahydro-4H-cyclopenta[d][1,3]dioxol-4-yl)benzyl)carbamate 122d (340 mg, 0.9 mmol, 1.0 eq) in MeOH (4.0 mL) was added NaBH₄ (36.0 mg, 0.9 mmol, 1.0 eq) in portions at 0° C. The reaction mixture was stirred at room temperature for 30 min then concentrated under reduced pressure. The residue was dissolved in DCM (5.0 mL), and purified by silica gel column chromatography (40:60 Petroleum ether/THF) to afford tert-butyl (3-((3aR,4R,6S,6aS)-6-hydroxy-2,2-dimethyltetrahydro-4H-cyclopenta[d][1,3]dioxol-4-yl)benzyl)carbamate 123d (330 mg, 96%) as a clear oil. LCMS (conditions: L-column3 ODS, 50*3.0 mm, 3.0 μm, Mobile Phase A: Water/5 mM NH₄HCO₃+0.05% Ammonia, Mobile Phase B: Acetonitrile, 5%-100% B-2 min, 1.50 L/min): T_{R}=1.38 min; ES m/z [M−H]: 362.

### Tert-butyl (3-((3aR,4R,6R,6aS)-6-(4-chloro-7H-pyrrolo[2,3-d]pyrimidin-7-yl)-2,2-dimethyltetrahydro-4H-cyclopenta[d][1,3]dioxol-4-yl)benzyl)carbamate (124d)

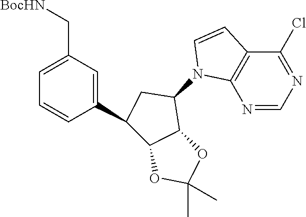 [SMILES: CC(C)(C)OC(=O)NCc1cccc([C@H]2C[C@@H](n3ccc4c(Cl)ncnc43)[C@@H]3OC(C)(C)O[C@@H]32)c1]

To a stirred solution of tert-butyl (3-((3aR,4R,6S,6aS)-6-hydroxy-2,2-dimethyltetrahydro-4H-cyclopenta[d][1,3]dioxol-4-yl)benzyl)carbamate 123d (0.31 g, 0.8 mmol, 1.2 eq), 4-chloro-7H-pyrrolo[2,3-d]pyrimidine (0.11 g, 0.7 mmol, 1.0 eq) and PPh₃ (0.37 g, 1.4 mmol, 2.0 eq) in toluene (2.5 mL) was added DBAD (0.33 g, 1.4 mmol, 2.0 eq) in toluene (2.5 mL) dropwise at 0° C. The reaction mixture was stirred at 50° C. for 2 h, then concentrated under reduced pressure. The residue was dissolved in DCM (3.0 mL) and purified by silica gel column chromatography (60:40 Petroleum ether/THF) to afford tert-butyl (3-((3aR,4R,6R,6aS)-6-(4-chloro-7H-pyrrolo[2,3-d]pyrimidin-7-yl)-2,2-dimethyltetrahydro-4H-cyclopenta[d][1,3]dioxol-4-yl)benzyl)carbamate 124d (220 mg, 61%) as a white solid. LCMS (conditions: L-column3 ODS, 50*3.0 mm, 3.0 μm, Mobile Phase A: Water/5 mM NH₄HCO₃+0.05% Ammonia, Mobile Phase B: Acetonitrile, 5%-100% B-3 min, 1.50 L/min): T_{R}=2.04 min; ES m/z [M+H]⁺: 499, 501.

### Tert-butyl (3-((3aR,4R,6R,6aS)-6-(4-((4-methoxybenzyl)amino)-7H-pyrrolo[2,3-d]pyrimidin-7-yl)-2,2-dimethyltetrahydro-4H-cyclopenta[d][1.3]dioxol-4-yl)benzyl)carbamate (125d)

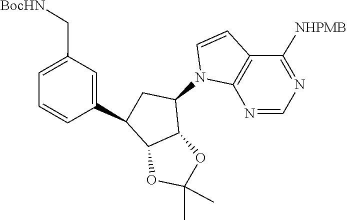

Into a 40 mL vial were added tert-butyl (3-((3aR,4R,6R,6aS)-6-(4-chloro-7H-pyrrolo[2,3-d]pyrimidin-7-yl)-2,2-dimethyltetrahydro-4H-cyclopenta[d][1,3]dioxol-4-yl)benzyl)carbamate 124d (0.21 g, 0.4 mmol, 1.0 eq), 4-methoxy-benzenemethanamine, (0.23 g, 1.7 mmol, 4.0 eq), TEA (0.13 g, 1.3 mmol, 3.0 eq) and EtOH (3.0 mL). The reaction mixture was stirred at 80° C. for 2 h, then allowed to cool to room temperature. The resulting mixture was concentrated under reduced pressure. The residue was dissolved in DCM (5.0 mL) and purified by silica gel column chromatography (1:1 Petroleum ether/THF) to afford tert-butyl (3-((3aR,4R,6R,6aS)-6-(4-((4-methoxybenzyl)amino)-7H-pyrrolo[2,3-d]pyrimidin-7-yl)-2,2-dimethyltetrahydro-4H-cyclopenta[d][1,3]dioxol-4-yl)benzyl)carbamate 125d (150 mg, 59%) as a white solid. LCMS (conditions: L-column3 ODS, 50*3.0 mm, 3.0 μm, Mobile Phase A: Water/5 mM NH₄HCO₃+0.05% Ammonia, Mobile Phase B: Acetonitrile, 5%-100% B-2.5 min, 1.50 L/min): T_{R}=1.88 min; ES m/z [M+H]⁺: 600.

### (1R,2S,3R,5R)-3-(4-Amino-7H-pyrrolo[2,3-d]pyrimidin-7-yl)-5-(3-(aminomethyl)phenyl)cyclopentane-1,2-diol (659)

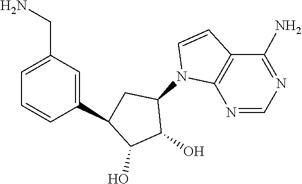 [SMILES: NCc1cccc([C@H]2C[C@@H](n3ccc4c(N)ncnc43)[C@H](O)[C@@H]2O)c1]

Into 40 mL vial were added tert-butyl (3-((3aR,4R,6R,6aS)-6-(4-((4-methoxybenzyl)amino)-7H-pyrrolo[2,3-d]pyrimidin-7-yl)-2,2-dimethyltetrahydro-4H-cyclopenta[d][1,3]dioxol-4-yl)benzyl)carbamate 125d (60 mg, 0.1 mmol, 1.0 eq), TFA (2.0 mL) and DCM (2.0 mL) at room temperature. The reaction mixture was stirred at 80° C. for 1 h, then allowed to cool to room temperature and concentrated under reduced pressure. The residue was dissolved in MeOH (3.0 mL) and basified to pH>8 with NH₃·H₂O. The mixture was purified by reverse phase flash chromatography with the following conditions (Column: Welch Utimate AQ-C18, 50*250 mm*10 μm; Mobile Phase A: Water (10 mmol/L NH₄HCO₃+0.1% NH₃·H₂O), Mobile Phase B: ACN; Flow rate: 80 mL/min; Gradient: 5% B to 60% B in 15 min, 60% B; Wavelength: 220 nm) to afford (1R,2S,3R,5R)-3-(4-amino-7H-pyrrolo[2,3-d]pyrimidin-7-yl)-5-(3-(aminomethyl)phenyl)cyclopentane-1,2-diol 659 (10.8 mg, 31%) as a white solid. LCMS (ZORBAX SB-Aq, 50*4.6 mm, 1.8 μm, Mobile Phase A: Water/0.02% TFA, Mobile Phase B: Acetonitrile, 5%-95% B-3 min, 1.50 L/min): T_{R}=0.93 min; ES m/z [M+H]⁺: 340. ¹H NMR (300 MHZ, DMSO-d₆+D₂O) δ8.06 (s, 1H), 7.40-7.10 (m, 5H), 6.58 (d, J=3.6 Hz, 1H), 5.00-4.77 (m, 1H), 4.29 (t, J=6.7 Hz, 1H), 4.14 (s, 1H), 4.06 (t, J=6.7 Hz, 1H), 3.71 (d, J=2.6 Hz, 1H), 3.15-2.99 (m, 1H), 2.39-2.28 (m, 1H), 2.09-1.88 (m, 1H).

### Synthesis of (1R,2S,3R,5R)-3-{4-amino-2-chloropyrrolo[2,3-d]pyrimidin-7-yl}-5-[3-(aminomethyl)phenyl]cyclopentane-1,2-diol (660)

### Tert-butyl N-({3-[(3aR,4R,6R,6aS)-6-{2,4-dichloropyrrolo[2,3-d]pyrimidin-7-yl}-2,2-dimethyl-tetrahydro-3aH-cyclopenta[d][1,3]dioxol-4-yl]phenyl}methyl)carbamate (126d)

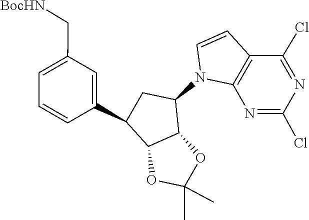 [SMILES: CC(C)(C)OC(=O)NCc1cccc([C@H]2C[C@@H](n3ccc4c(Cl)nc(Cl)nc43)[C@@H]3OC(C)(C)O[C@@H]32)c1]

Into a 40 mL vial was placed a solution of tert-butyl N-({3-[(3aR,4R,6S,6aS)-6-hydroxy-2,2-dimethyl-tetrahydro-3aH-cyclopenta[d][1,3]dioxol-4-yl]phenyl}methyl)carbamate (0.41 g, 1.1 mmol, 1.4 eq) in THF (2.0 mL). 2,4-Dichloro-7H-pyrrolo[2,3-d]pyrimidine (0.15 g, 0.8 mmol, 1.0 eq) and PPh₃ (0.42 g, 1.6 mmol, 2.0 eq) were added, followed by DIAD (0.32 g, 1.6 mmol, 2.0 eq) dropwise over 2 min at 0° C. The resulting mixture was stirred for 10 h at 40° C., then concentrated under reduced pressure. The residue was dissolved in DCM (2.0 ml) and purified by silica gel column chromatography (70:30 Petroleum ether: THF) to afford tert-butyl N-({3-[(3aR,4R,6R,6aS)-6-{2,4-dichloropyrrolo[2,3-d]pyrimidin-7-yl}-2,2-dimethyl-tetrahydro-3aH-cyclopenta[d][1,3]dioxol-4-yl]phenyl}methyl)carbamate 126d (280 mg, 65%) as a colorless oil. LCMS (conditions: Kinetex EVO C18, 50*3.0 mm, 2.6 μm, Mobile Phase A: Water/5 mM NH₄HCO₃+0.05% Ammonia, Mobile Phase B: Acetonitrile/5% water, 2%-100% B-3 min, 1.50 L/min): T_{R}=2.06 min; ES m/z [M+H]⁺: 533, 535.

### Tert-butyl N-({3-[(3aR,4R,6R,6aS)-6-{4-amino-2-chloropyrrolo[2,3-d]pyrimidin-7-yl}-2,2-dimethyl-tetrahydro-3aH-cyclopenta[d][1,3]dioxol-4-yl]phenyl}methyl)carbamate (127d)

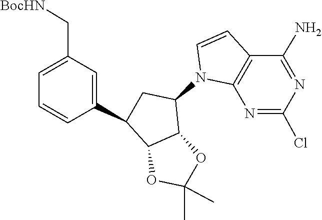 [SMILES: CC(C)(C)OC(=O)NCc1cccc([C@H]2C[C@@H](n3ccc4c(N)nc(Cl)nc43)[C@@H]3OC(C)(C)O[C@@H]32)c1]

Into a 40 mL vial was placed a solution of tert-butyl N-({3-[(3aR,4R,6R,6aS)-6-{2,4-dichloropyrrolo[2,3-d]pyrimidin-7-yl}-2,2-dimethyl-tetrahydro-3aH-cyclopenta[d][1,3]dioxol-4-yl]phenyl}methyl)carbamate 126d (0.22 g, 0.4 mmol, 1.0 eq) in 7M NH₃ in MeOH (2.0 mL). The mixture was stirred for 8 h at 80° C. The resulting mixture was concentrated under vacuum to afford tert-butyl N-({3-[(3aR,4R,6R,6aS)-6-{4-amino-2-chloropyrrolo[2,3-d]pyrimidin-7-yl}-2,2-dimethyl-tetrahydro-3aH-cyclopenta[d][1,3]dioxol-4-yl]phenyl}methyl)carbamate 127d (180 mg, crude) as a yellow solid. LCMS (Conditions: L-column3 ODS, 50*3.0 mm, 3.0 μm, Mobile PhaseA: Water/5 mM NH₄HCO₃+0.05% Ammonia, Mobile Phase B: Acetonitrile/5% water, 2%-100% B-3 min, 1.50 L/min): T_{R}=2.01 min; ES m/z [M+H]⁺: 514, 516.

### (1R,2S,3R,5R)-3-{4-Amino-2-chloropyrrolo[2,3-d]pyrimidin-7-yl}-5-[3-(aminomethyl)phenyl]cyclopentane-1,2-diol (660)

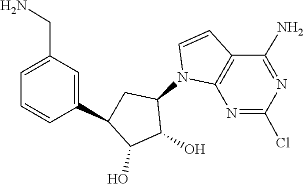 [SMILES: NCc1cccc([C@H]2C[C@@H](n3ccc4c(N)nc(Cl)nc43)[C@H](O)[C@@H]2O)c1]

Into a 40 mL vial was placed a solution of tert-butyl N-({3-[(3aR,4R,6R,6aS)-6-{4-amino-2-chloropyrrolo[2,3-d]pyrimidin-7-yl}-2,2-dimethyl-tetrahydro-3aH-cyclopenta[d][1,3]dioxol-4-yl]phenyl}methyl)carbamate 127d (0.15 g, 0.3 mmol, 1.0 eq) in 4M HCl in MeOH (2.0 mL). The mixture was stirred for 20 min at 50° C. The resulting mixture was concentrated under vacuum. The residue was dissolved in MeOH (2.0 mL) and basified to pH>8 with Na₂CO₃. The resulting mixture was filtered and the filter cake washed with MeOH (3×1 mL). The combined filtrate was purified by Prep-HPLC (Column: Spherical C18, 20-40 μm, 120g; Mobile Phase A: Water/5 mM NH₄HCO₃+0.05% Ammonia, Mobile Phase B: ACN; Flow rate: 80 mL/min; Gradient: 10% B to 35% B over 10 min; Wavelength: 254 nm; Number Of Runs: 2) to afford (1R,2S,3R,5R)-3-{4-amino-2-chloropyrrolo[2,3-d]pyrimidin-7-yl}-5-[3-(aminomethyl)phenyl]cyclopentane-1,2-diol 660 (80 mg, 73%) as an off-white solid. LCMS (Cortecs C18+, 50*3.0 mm, 2.7 μm, Mobile Phase A: Water/0.02% TFA, Mobile Phase B: Acetonitrile, 5%-95% B-3 min, 1.50 L/min): T_{R}=0.68 min; ES m/z [M+H]⁺: 374, 376. ¹H NMR (400 MHZ, DMSO-d₆) δ7.41-7.36 (m, 1H), 7.35-7.22 (m, 3H), 7.21-7.15 (m, 1H), 6.60 (d, J=3.5 Hz, 1H), 4.86-4.76 (m, 1H), 4.26 (t, J=6.8 Hz, 1H), 4.03 (t, J=6.3 Hz, 1H), 3.70 (s, 2H), 3.13-3.02 (m, 1H), 2.40-2.28 (m, 1H), 1.97 (q, J=11.5 Hz, 1H).

### Synthesis of (1R,2S,3R,5R)-3-(4-amino-2-chloro-7H-pyrrolo[2,3-d]pyrimidin-7-yl)-5-(1-methyl-1H-pyrazol-4-yl)cyclopentane-1,2-diol (661)

### (3aR,6R,6aR)-2,2-Dimethyl-6-(1-methyl-1H-pyrazol-4-yl)tetrahydro-4H-cyclopenta[d][1,3]dioxol-4-one (86d)

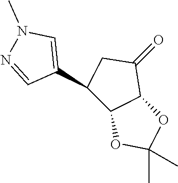 [SMILES: Cn1cc([C@H]2CC(=O)[C@@H]3OC(C)(C)O[C@H]23)cn1]

Into a 40 mL vial were added (3aR,6aR)-2,2-dimethyl-3aH, 6aH-cyclopenta[d][1,3]dioxol-4-one (0.30 g, 1.9 mmol, 1.0 eq), 1-methyl-4-(4,4,5,5-tetramethyl-1,3,2-dioxaborolan-2-yl)pyrazole (0.60 g, 2.9 mmol, 1.5 eq), cyclooctadiene rhodium chloride dimer (95.95 mg, 0.195 mmol, 0.1 eq), 3M aqueous KOH (130 μL) and CH₃OH (3.0 mL) under N₂ atmosphere. The reaction was irradiated with microwave radiation for 2 h at 40° C. The mixture was allowed to cool and was concentrated under reduced pressure. The residue was dissolved in DCM (3.0 mL) and purified by silica gel column chromatography (30:70 Petroleum ether/THF) to afford (3aR,6R,6aR)-2,2-dimethyl-6-(1-methyl-1H-pyrazol-4-yl)tetrahydro-4H-cyclopenta[d][1,3]dioxol-4-one 86d (200 mg, 43%) as a yellow oil. LCMS (conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-2 min, 1.50 L/min): T_{R}=1.10 min, ES m/z [M+H]⁺: 237.

### (3aS,4S,6R,6aR)-2,2-dimethyl-6-(1-methyl-1H-pyrazol-4-yl)tetrahydro-4H-cyclopenta[d][1,3]dioxol-4-ol (87d)

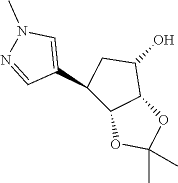

To a stirred solution of (3aR,6R,6aR)-2,2-dimethyl-6-(1-methyl-1H-pyrazol-4-yl)tetrahydro-4H-cyclopenta[d][1,3]dioxol-4-one 87d (0.18 g, 0.8 mmol, 1.0 eq) in MeOH (3.0 mL) was added NaBH₄ (58.0 mg, 1.5 mmol, 2.0 eq) in portions at 0° C. The reaction mixture was stirred at room temperature for 20 min. The resulting mixture was concentrated under reduced pressure. The residue was dissolved in DCM (3.0 mL) and purified by silica gel column chromatography (60:40 Petroleum ether/THF) to afford (3aS,4S,6R,6aR)-2,2-dimethyl-6-(1-methyl-1H-pyrazol-4-yl)tetrahydro-4H-cyclopenta[d][1,3]dioxol-4-ol 88d (160 mg, 88%) as a white solid. LCMS (conditions L-column3 ODS, 50*3.0 mm, 3.0 μm, Mobile Phase Water/5 mM NH₄HCO₃+0.05% Ammonia, Mobile Phase B: Acetonitrile/5% water, 5%-100% B-2.5 min, 1.50 L/min): T_{R}=1.03 min, ES m/z [M+H]⁺: 239.

### 4-[(3aR,4R,6R,6aS)-6-{2,4-Dichloropyrrolo[2,3-d]pyrimidin-7-yl}-2,2-dimethyl-tetrahydro-3aH-cyclopenta[d][1,3]dioxol-4-yl]-1-methylpyrazole (89d)

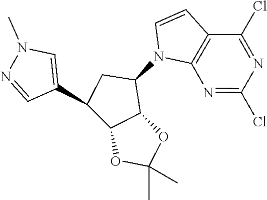 [SMILES: Cn1cc([C@H]2C[C@@H](n3ccc4c(Cl)nc(Cl)nc43)[C@@H]3OC(C)(C)O[C@@H]32)cn1]

To a stirred solution of 2,4-dichloro-7H-pyrrolo[2,3-d]pyrimidine (0.10 g, 0.5 mmol, 1.0 eq), (3aS,4S,6R,6aR)-2,2-dimethyl-6-(1-methyl-1H-pyrazol-4-yl)tetrahydro-4H-cyclopenta[d][1,3]dioxol-4-ol 88d (0.16 g, 0.7 mmol, 1.3 eq) and PPh₃ (0.28 g, 1.1 mmol, 2.0 eq) in THF (3.0 mL) was added DIAD (0.21 g, 1.1 mmol, 2.0 eq) dropwise at 0° C. under N₂ atmosphere. The reaction was stirred at room temperature for 4 h. The resulting mixture was concentrated under reduced pressure. The residue was dissolved in DCM (3.0 mL) and purified by silica gel column chromatography (50:50 Petroleum ether/THF) to afford 4-[(3aR,4R,6R,6aS)-6-{2,4-dichloropyrrolo[2,3-d]pyrimidin-7-yl}-2,2-dimethyl-tetrahydro-3aH-cyclopenta[d][1,3]dioxol-4-yl]-1-methylpyrazole 89d (80 mg, 36%) as a white solid. LCMS (conditions Cortecs C18+, 50*3.0 mm, 2.7 μm, Mobile Phase A: Water/0.05% TFA, Mobile Phase B: Acetonitrile, 2%-100% B-2 min, 1.50 L/min): T_{R}=1.10 min, ES m/z [M+H]⁺: 408, 410.

### 2-chloro-7-((3aS,4R,6R,6aR)-2,2-dimethyl-6-(1-methyl-1H-pyrazol-4-yl)tetrahydro-4H-cyclopenta[d][1,3]dioxol-4-yl)-7H-pyrrolo[2,3-d]pyrimidin-4-amine (89d)

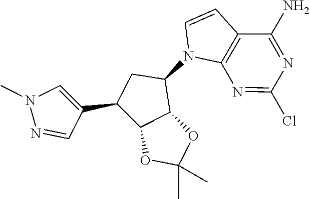 [SMILES: Cn1cc([C@H]2C[C@@H](n3ccc4c(N)nc(Cl)nc43)[C@@H]3OC(C)(C)O[C@@H]32)cn1]

Into a 40 mL vial were added 4-[(3aR,4R,6R,6aS)-6-{2,4-dichloropyrrolo[2,3-d]pyrimidin-7-yl}-2,2-dimethyl-tetrahydro-3aH-cyclopenta[d][1,3]dioxol-4-yl]-1-methylpyrazole 88d (70 mg, 0.17 mmol, 1.0 eq) and 7M NH₃ in MeOH (4.0 mL) at room temperature. The reaction was stirred at 80° C. for 3 h. The mixture was allowed to cool to room temperature, then concentrated under reduced pressure to afford 2-chloro-7-((3aS,4R,6R,6aR)-2,2-dimethyl-6-(1-methyl-1H-pyrazol-4-yl)tetrahydro-4H-cyclopenta[d][1,3]dioxol-4-yl)-7H-pyrrolo[2,3-d]pyrimidin-4-amine 89d (40 mg, 60%), which was used in the next step directly without further purification. LCMS (conditions Cortecs C18+, 50*3.0 mm,, 2.7 μm, Mobile Phase A: Water/0.05% TFA, Mobile Phase B: Acetonitrile, 2%-95% B-3 min, 1.50 L/min): T_{R}=1.14 min, ES m/z [M+H]⁺: 389, 391

### (1R,2S,3R,5R)-3-(4-Amino-2-chloro-7H-pyrrolo[2,3-d]pyrimidin-7-yl)-5-(1-methyl-1H-pyrazol-4-yl)cyclopentane-1,2-diol (661)

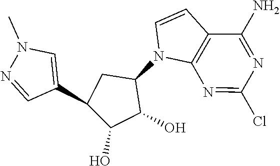 [SMILES: Cn1cc([C@H]2C[C@@H](n3ccc4c(N)nc(Cl)nc43)[C@H](O)[C@@H]2O)cn1]

Into a 40 mL vial were added 2-chloro-7-((3aS,4R,6R,6aR)-2,2-dimethyl-6-(1-methyl-1H-pyrazol-4-yl)tetrahydro-4H-cyclopenta[d][1,3]dioxol-4-yl)-7H-pyrrolo[2,3-d]pyrimidin-4-amine 89d (40 mg, 0.1 mmol, 1.0 eq) and 4M HCl in MeOH (3.0 mL) at room temperature. The reaction was stirred for 40 min. The mixture was allowed to cool to room temperature, then concentrated under reduced pressure. The residue was dissolved in DCM (3.0 mL) and basified to pH >8 with NH₃·H₂O. The mixture was purified by Prep-HPLC with the following conditions (Column: XBridge Prep C18 OBD Column, 19*150 mm, 5 μm; Mobile Phase A: Water (10 mmol/L NH₄HCO₃+0.1% NH₃·H₂O), Mobile Phase B: ACN; Flow rate: 30 mL/min; Gradient: 10% B to 60% B in 8 min, 60% B; Wavelength: 254/220 nm) to afford (1R,2S,3R,5R)-3-{4-amino-2-chloropyrrolo[2,3-d]pyrimidin-7-yl}-5-(1-methylpyrazol-4-yl)cyclopentane-1,2-diol 661 (12.4 mg, 34%) as a white solid. LCMS (Cortecs C18+, 50*3.0 mm, 2.7 μm, 2.7 μm, Mobile Phase A: Water/0.02% TFA, Mobile Phase B: Acetonitrile, 5%-95% B-3 min, 1.50 L/min): T_{R}=0.79 min, ES m/z [M+H]⁺: 349, 351. ¹H NMR (400 MHZ, DMSO-d₆) δ7.64 (s, 1H), 7.45 (s, 2H), 7.39 (s, 1H), 7.29 (d, J=3.6 Hz, 1H), 6.58 (d, J=3.5 Hz, 1H), 4.91-4.85 (m, 2H), 4.80 (dd, J=10.4, 7.6 Hz, 1H), 4.21 (q, J=6.3 Hz, 1H), 3.85 (q, J=5.8 Hz, 1H), 3.80 (s, 3H), 3.04-2.93 (m, 1H), 2.47-2.35 (m, 1H), 1.91-1.78 (m, 1H).

### Synthesis of 4-[(1R,2R,3S,4R)-4-{4-amino-2-chloropyrrolo[2,3-d]pyrimidin-7-yl}-2,3-dihydroxycyclopentyl]-1H-pyridin-2-one (662)

### (3aR,6R,6aR)-6-(2-Methoxypyridin-4-yl)-2,2-dimethyl-tetrahydrocyclopenta[d][1,3]dioxol-4-one (134d)

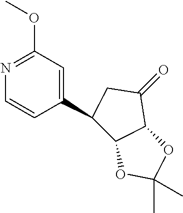 [SMILES: COc1cc([C@H]2CC(=O)[C@@H]3OC(C)(C)O[C@H]23)ccn1]

Into a 40 mL vial was placed a solution of (3aR,6aR)-2,2-dimethyl-3aH,6aH-cyclopenta[d][1,3]dioxol-4-one (0.50 g, 3.3 mmol, 1.0 eq) in 1,4-dioxane (4.0 mL) and H₂O (1.0 mL). 2-Methoxypyridin-4-ylboronic acid (0.70 g, 4.9 mmol, 1.5 eq), acetylacetonato bis(ethylene) rhodium (I)(83.72 mg, 0.3 mmol, 0.1 eq) and (R)-BINAP (0.21 g, 0.3 mmol, 0.1 eq) were added. The mixture was stirred for 3 h at 100° C., and the resulting mixture was concentrated under vacuum. The residue was dissolved in DCM (5.0 mL) and purified by silica gel column chromatography (85:15 Petroleum ether: THF) to afford (3aR,6R,6aR)-6-(2-methoxypyridin-4-yl)-2,2-dimethyl-tetrahydrocyclopenta[d][1,3]dioxol-4-one 134d (400 mg, 46%) as light yellow oil. LCMS (conditions Kinetex EVO C18, 50*3.0 mm, 2.6 μm, Mobile PhaseA: Water/5 mM NH₄HCO₃+0.05% Ammonia, Mobile Phase B: Acetonitrile/5% water, 2%-100% B-3 min, 1.50 L/min): T_{R}=1.60 min; ES m/z [M+H]⁺: 264.

### (3aS,45,6R,6aR)-6-(2-Methoxypyridin-4-yl)-2,2-dimethyl-tetrahydro-3aH-cyclopenta[d][1,3]dioxol-4-ol (135d)

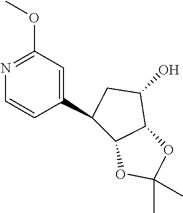 [SMILES: COc1cc([C@H]2C[C@H](O)[C@@H]3OC(C)(C)O[C@@H]32)ccn1]

Into a 40 mL vial was placed a solution of (3aR,6R,6aR)-6-(2-methoxypyridin-4-yl)-2,2-dimethyl-tetrahydrocyclopenta[d][1,3]dioxol-4-one 134d (0.40 g, 1.5 mmol, 1.0 eq) in MeOH (4.0 mL) which was treated with NaBH₄ (57.5 mg, 1.5 mmol, 1.0 eq) in portions at room temperature. The resulting mixture was stirred for 20 min then concentrated under reduced pressure. The residue was dissolved in DCM (2.0 mL) and purified by silica gel column chromatography (94:6 DCM: MeOH) to afford (3aS,4S,6R,6aR)-6-(2-methoxypyridin-4-yl)-2,2-dimethyl-tetrahydro-3aH-cyclopenta[d][1,3]dioxol-4-ol 135d (300 mg, 74%) as light yellow oil. LCMS (conditions: L-column3 ODS, 50*3.0 mm, 3.0 μm, Mobile Phase A: Water/5 mM NH₄HCO₃+0.05% Ammonia, Mobile Phase B: Acetonitrile/5% water, 2%-100% B-3 min, 1.50 L/min): T_{R}=1.35 min; ES m/z [M+H]⁺: 266. ¹H NMR (400 MHZ, DMSO-d₆) δ8.07 (d, J=5.3 Hz, 1H), 6.86-6.82 (m, 1H), 6.63 (s, 1H), 4.63-4.58 (m, 1H), 4.55-4.46 (m, 2H), 3.99-3.91 (m, 1H), 3.83 (s, 3H), 3.19-3.13 (m, 1H), 2.15-2.05 (m, 1H), 1.85-1.73 (m, 1H), 1.46 (s, 3H), 1.28 (s, 3H).

### 4-[(3aR,4R,6R,6aS)-6-{2,4-Dichloropyrrolo[2,3-d]pyrimidin-7-yl}-2,2-dimethyl-tetrahydro-3aH-cyclopenta[d][1,3]dioxol-4-yl]-2-methoxypyridine (136d)

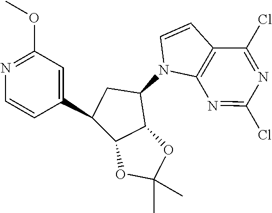 [SMILES: COc1cc([C@H]2C[C@@H](n3ccc4c(Cl)nc(Cl)nc43)[C@@H]3OC(C)(C)O[C@@H]32)ccn1]

Into a 40 ml vial was placed a solution of (3aS,4S,6R,6aR)-6-(2-methoxypyridin-4-yl)-2,2-dimethyl-tetrahydro-3aH-cyclopenta[d][1,3]dioxol-4-ol 135d (0.27 g, 1.0 mmol, 1.3 eq) in THF (3.0 mL). 2,4-Dichloro-7H-pyrrolo[2,3-d]pyrimidine (0.15 g, 0.8 mmol, 1.0 eq) and PPh₃ (0.42 g, 1.6 mmol, 2.0 eq) were added, followed by DIAD (0.32 g, 1.6 mmol, 2.0 eq) dropwise over 5 min at 0° C. The resulting mixture was stirred for 8 h at 40° C., then concentrated under vacuum. The residue was dissolved in DCM (1.0 mL) and purified by silica gel column chromatography (85:15 DCM: THF) to afford 4-[(3aR,4R,6R,6aS)-6-{2,4-dichloropyrrolo[2,3-d]pyrimidin-7-yl}-2,2-dimethyl-tetrahydro-3aH-cyclopenta[d][1,3]dioxol-4-yl]-2-methoxypyridine 136d (300 mg, 86%) as a light brown oil. LCMS (conditions: L-column3 ODS, 50*3.0 mm, 3.0 μm, Mobile Phase A: Water/5 mM NH₄HCO₃+0.05% Ammonia, Mobile Phase B: Acetonitrile/5% water, 2%-100% B-3 min, 1.50 L/min): T_{R}=2.09 min; ES m/z [M+H]⁺: 435; 437.

### 4-[(3aR,4R,6R,6aS)-6-{2,4-Dichloropyrrolo[2,3-d]pyrimidin-7-yl}-2,2-dimethyl-tetrahydro-3aH-cyclopenta[d][1,3]dioxol-4-yl]-1H-pyridin-2-one (137d)

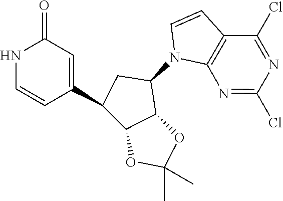 [SMILES: CC1(C)O[C@@H]2[C@H](O1)[C@@H](c1cc[nH]c(=O)c1)C[C@H]2n1ccc2c(Cl)nc(Cl)nc21]

Into an 8 mL vial was placed a solution of 4-[(3aR,4R,6R,6aS)-6-{2,4-dichloropyrrolo[2,3-d]pyrimidin-7-yl}-2,2-dimethyl-tetrahydro-3aH-cyclopenta[d][1,3]dioxol-4-yl]-2-methoxypyridine 136d (0.20 g, 0.4 mmol, 1.0 eq) in MeCN (2.0 mL). Chlorotrimethylsilane (99.8 mg, 0.9 mmol, 2.0 eq) and Nal (0.21 g, 1.4 mmol, 3.0 eq) were added. The mixture was irradiated with microwave radiation for 10 min at 100° C., then quenched with saturated aqueous NaS₂O₃ solution at 0° C. The resulting mixture was extracted with AcOEt (3×5.0 mL). The combined organic layers were washed with brine and dried over anhydrous Na₂SO₄. After filtration, the filtrate was concentrated under reduced pressure and purified by silica gel column chromatography (94:6 DCM/MeOH) to afford 4-[(3aR,4R,6R,6aS)-6-{2,4-dichloropyrrolo[2,3-d]pyrimidin-7-yl}-2,2-dimethyl-tetrahydro-3aH-cyclopenta[d][1,3]dioxol-4-yl]-1H-pyridin-2-one 137d (100 mg, 51%) as a light yellow oil. LCMS (Conditions: L-column3 ODS, 50*3.0 mm, 3.0 μm, Mobile Phase A: Water/5 mM NH₄HCO_{3 +0.05}% Ammonia, Mobile Phase B: Acetonitrile/5% water, 2%-100% B-3 min, 1.50 L/min): T_{R}=1.61 min; ES m/z [M+H]⁺: 421, 422.

### 4-[(3aR,4R,6R,6aS)-6-{4-Amino-2-chloropyrrolo[2,3-d]pyrimidin-7-yl}-2,2-dimethyl-tetrahydro-3aH-cyclopenta[d][1,3]dioxol-4-yl]-1H-pyridin-2-one (138d)

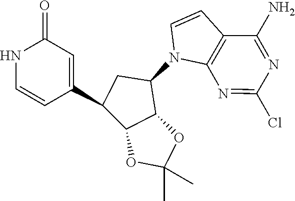 [SMILES: CC1(C)O[C@@H]2[C@H](O1)[C@@H](c1cc[nH]c(=O)c1)C[C@H]2n1ccc2c(N)nc(Cl)nc21]

Into an 8 mL vial was placed a solution of 4-[(3aR,4R,6R,6aS)-6-{2,4-dichloropyrrolo[2,3-d]pyrimidin-7-yl}-2,2-dimethyl-tetrahydro-3aH-cyclopenta[d][1,3]dioxol-4-yl]-1H-pyridin-2-one 137d (50 mg, 0.1 mmol, 1.0 eq) in 7M NH₃ in MeOH (2.0 mL). The mixture was stirred for 4 h at 80° C. The resulting mixture was concentrated under reduced pressure and dissolved in MeOH (2.0 mL) and purified by Prep-HPLC (Column: YMC-Actus Triart C18, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 15% B to 60% B over 10 min, Wavelength: 220 nm) to afford 4-[(3aR,4R,6R,6aS)-6-{4-amino-2-chloropyrrolo[2,3-d]pyrimidin-7-yl}-2,2-dimethyl-tetrahydro-3aH-cyclopenta[d][1,3]dioxol-4-yl]-1H-pyridin-2-one 138d (30 mg, 62%) as an off-white solid. LCMS (Conditions: L-column3 ODS, 50*3.0 mm, 3.0 μm, Mobile Phase A: Water/5 mM NH₄HCO₃+0.05% Ammonia, Mobile Phase B: Acetonitrile/5% water, 2%-100% B-3 min, 1.50 L/min): T_{R}=1.29 min; ES m/z [M+H]⁺: 402, 404.

### 4-[(1R,2R,3S,4R)-4-{4-Amino-2-chloropyrrolo[2,3-d]pyrimidin-7-yl}-2,3-dihydroxycyclopentyl]-1H-pyridin-2-one (662)

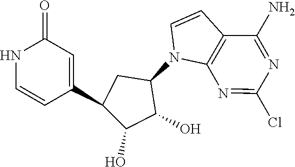 [SMILES: Nc1nc(Cl)nc2c1ccn2[C@@H]1C[C@H](c2cc[nH]c(=O)c2)[C@@H](O)[C@H]1O]

Into a 40 mL vial was placed a solution of 4-[(3aR,4R,6R,6aS)-6-{4-amino-2-chloropyrrolo[2,3-d]pyrimidin-7-yl}-2,2-dimethyl-tetrahydro-3aH-cyclopenta[d][1,3]dioxol-4-yl]-1H-pyridin-2-one 138d (30.0 mg, 0.1 mmol, 1.0 eq) in 4M HCl in MeOH (2.0 mL). The mixture was stirred for 10 min at 60° C., then concentrated under reduced pressure. The residue was dissolved in MeOH (2.0 mL) and neutralized to pH>8 with NaHCO₃. The resulting mixture was filtered and purified by Prep-HPLC (Column: YMC-Actus Triart C18, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 10% B to 50% B over 10 min, Wavelength: 254 nm) to afford 4-[(1R,2R,3S,4R)-4-{4-amino-2-chloropyrrolo[2,3-d]pyrimidin-7-yl}-2,3-dihydroxycyclopentyl]-1H-pyridin-2-one 662 (6.2 mg, 22%) as an off-white solid. LCMS (ZORBAX SB-Aq, 50*4.6 mm, 1.8 μm, Mobile Phase A: Water/0.02% TFA, Mobile Phase B: Acetonitrile, 5%-95% B-3 min, 1.50 L/min): T_{R}=1.16 min; ES m/z [M+H]⁺: 362, 364. 1H NMR (400 MHZ, DMSO-d₆) δ 11.38 (br, s, 1H), 7.45 (s, 2H), 7.38 (d, J=3.6 Hz, 1H), 7.31 (d, J=6.8 Hz, 1H), 6.59 (d, J=3.6 Hz, 1H), 6.33-6.24 (m, 2H), 5.01-4.95 (m, 2H), 4.85-4.73 (m, 1H), 4.17 (q, J=6.4 Hz, 1H), 4.04 (q, J=6.6 Hz, 1H), 2.96-2.85 (m, 1H), 2.36-2.22 (m, 1H), 1.90 (q, J=11.7 Hz, 1H).

### (1R,2S,3R,5R)-3-{4-Amino-2-chloropyrrolo[2,3-d]pyrimidin-7-yl}-5-(2-methoxypyridin-4-yl)cyclopentane-1,2-diol (663)

### 7-[(3aS,4R,6R,6aR)-6-(2-Methoxypyridin-4-yl)-2,2-dimethyl-tetrahydro-3aH-cyclopenta[d][1,3]dioxol-4-yl]-2-chloropyrrolo[2,3-d]pyrimidin-4-amine (139d)

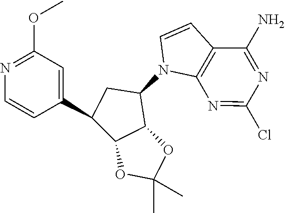 [SMILES: COc1cc([C@H]2C[C@@H](n3ccc4c(N)nc(Cl)nc43)[C@@H]3OC(C)(C)O[C@@H]32)ccn1]

Into an 8 mL vial was placed a solution of 4-[(3aR,4R,6R,6aS)-6-{2,4-dichloropyrrolo[2,3-d]pyrimidin-7-yl}-2,2-dimethyl-tetrahydro-3aH-cyclopenta[d][1,3]dioxol-4-yl]-2-methoxypyridine 136d (0.15 g, 0.3 mmol, 1.0 eq) in 7M NH₃ in MeOH (2.0 mL), and the mixture stirred for 8 h at 80° C. The resulting mixture was concentrated under reduced pressure to afford 7-[(3aS,4R,6R,6aR)-6-(2-methoxypyridin-4-yl)-2,2-dimethyl-tetrahydro-3aH-cyclopenta[d][1,3]dioxol-4-yl]-2-chloropyrrolo[2,3-d]pyrimidin-4-amine 139d (30 mg, crude) as a light yellow solid. The mixture was used directly in the next step. LCMS (conditions: L-column3 ODS, 50*3.0 mm, 3.0 μm, Mobile PhaseA: Water/5 mM NH₄HCO₃+0.05% Ammonia, Mobile Phase B: Acetonitrile/5% water, 2%-100% B-3 min, 1.50 L/min): T_{R}=1.76 min; ES m/z [M+H]⁺: 416, 418.

### (1R,2S,3R,5R)-3-{4-Amino-2-chloropyrrolo[2,3-d]pyrimidin-7-yl}-5-(2-methoxypyridin-4-yl)cyclopentane-1,2-diol (663)

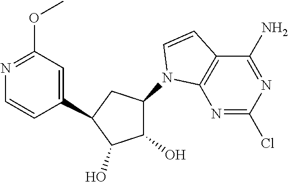 [SMILES: COc1cc([C@H]2C[C@@H](n3ccc4c(N)nc(Cl)nc43)[C@H](O)[C@@H]2O)ccn1]

Into a 40 mL vial was placed a solution of 7-[(3aS,4R,6R,6aR)-6-(2-methoxypyridin-4-yl)-2,2-dimethyl-tetrahydro-3aH-cyclopenta[d][1,3]dioxol-4-yl]-2-chloropyrrolo[2,3-d]pyrimidin-4-amine 139d (30 mg, 0.1 mmol, 1.0 eq) in 4M HCl in MeOH (2.0 mL). The mixture was stirred for 10 min at 60° C., then concentrated under reduced pressure. The residue was dissolved in MeOH (2.0 mL) and neutralized to pH>8 with NaHCO₃. The resulting mixture was filtered and the filtrate purified by Prep-HPLC (Column: YMC-Actus Triart C18, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 10% B to 45% B over 10 min, Wavelength: 254 nm) to afford (1R,2S,3R,5R)-3-{4-amino-2-chloropyrrolo[2,3-d]pyrimidin-7-yl}-5-(2-methoxypyridin-4-yl)cyclopentane-1,2-diol 663 (13.1 mg, 48%) as an off-white solid. LCMS (Cortecs C18+, 50*3.0 mm, 2.7 μm, Mobile Phase A: Water/0.02% TFA, Mobile Phase B: Acetonitrile, 5%-95% B-3 min, 1.50 L/min): T_{R}=0.89 min; ES m/z [M+H]⁺: 376, 378. ¹H NMR (400 MHZ, DMSO-d₆+D₂O) δ8.10 (d, J=5.3 Hz, 1H), 7.46 (s, 2H), 7.39 (d, J=3.6 Hz, 1H), 7.06 (dd, J=5.4, 1.5 Hz, 1H), 6.83 (s, 1H), 6.59 (d, J=3.5 Hz, 1H), 5.05-4.95 (m, 2H), 4.90-4.78 (m, 1H), 4.21 (q, J=6.3 Hz, 1H), 4.07 (q, J=6.5 Hz, 1H), 3.85 (s, 3H), 3.14-3.02 (m, 1H), 2.40-2.31 (m, 1H), 1.97 (q, J=11.7 Hz, 1H).

### Synthesis of (1R,2S,3R,5R)-3-{4-amino-2-chloropyrrolo[2,3-d]pyrimidin-7-yl]-5-(3,5-dimethoxyphenyl)cyclopentane-1,2-diol (664)

### (3aR,6R,6aR)-6-(3,5-Dimethoxyphenyl)-2,2-dimethyl-tetrahydrocyclopenta[d][1,3]dioxol-4-one (248d)

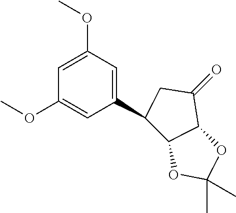

A solution of (3aR,6aR)-2,2-dimethyl-3aH,6aH-cyclopenta[d][1,3]dioxol-4-one (0.5 g, 3.2 mmol, 1.0 eq), 3,5-dimethoxyphenylboronic acid (0.77 g, 4.2 mmol, 1.3 eq), acetylacetonato bis(ethylene) rhodium (I)(83.7 mg, 0.3 mmol, 0.1 eq) and R-BINAP (0.26 g, 0.3 mmol, 0.1 eq) in 1,4-dioxane (5 mL) and H₂O (0.5 mL) was stirred for 3 h at 100° C. under nitrogen atmosphere. The reaction was quenched with water (10 mL) and then extracted with EtOAc (3×10 mL). The organic layer was dried over anhydrous Na₂SO₄, filtered and the filtrate was concentrated. The residue was purified by silica gel column chromatography (3:1 Petroleum ether: AcOEt) to afford (3aR,6R,6aR)-6-(3,5-dimethoxyphenyl)-2,2-dimethyl-tetrahydrocyclopenta[d][1,3]dioxol-4-one 248d (0.84 g, 89%) as a white solid. LCMS (conditions XBridge C18, 50*3.0 mm, 3.5 μm, Mobile Phase A: Water/5 mM NH₄HCO₃, Mobile Phase B: Methanol, 10-95% B-3 min, 1.50 mL/min): T_{R}=1.84 min; ES m/z [M+H]⁺: 293.

### (3aS,4S,6R,6aR)-6-(3,5-Dimethoxyphenyl)-2,2-dimethyl-tetrahydro-3aH-cyclopenta[d][1,3]dioxol-4-ol (249d)

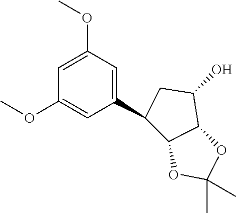

Into a 40 mL vial were added (3aR,6R,6aR)-6-(3,5-dimethoxyphenyl)-2,2-dimethyl-tetrahydrocyclopenta[d][1,3]dioxol-4-one 248d (0.84 g, 2.9 mmol, 1.0 eq) and NaBH₄ (0.16 g, 4.4 mmol, 1.5 eq) in MeOH (8.0 mL) at 0° C. The resulting mixture was stirred for 0.5 h at room temperature. The reaction was quenched with water (10 mL) and then extracted with EtOAc (3×10 mL). The organic layer was dried over anhydrous Na₂SO₄, filtered and the filtrate was concentrated. The residue was purified by silica gel column chromatography (2:1 Petroleum ether: AcOEt) to afford (3aS,4S,6R,6aR)-6-(3,5-dimethoxyphenyl)-2,2-dimethyl-tetrahydro-3aH-cyclopenta[d][1,3]dioxol-4-ol 249d (0.68 g, 80%) as a white solid. LCMS (conditions XBridge C18, 50*3.0 mm, 3.5 μm, Mobile Phase A: Water/5 mM NH₄HCO₃, Mobile Phase B: Acetonitrile, 10-95% B-3 min, 1.50 mL/min): T_{R}=1.62 min; ES m/z [M+H]⁺: 295.

### 7-[(3aS,4R,6R,6aR)-6-(3,5-Dimethoxyphenyl)-2,2-dimethyl-tetrahydro-3aH-cyclopenta[d][1,3]dioxol-4-yl]-2,4-dichloropyrrolo[2,3-d]pyrimidine (250d)

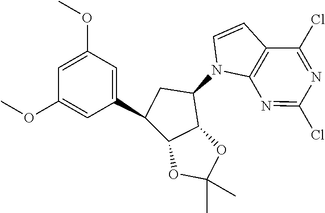 [SMILES: COc1cc(OC)cc([C@H]2C[C@@H](n3ccc4c(Cl)nc(Cl)nc43)[C@@H]3OC(C)(C)O[C@@H]32)c1]

A solution of (3aS,4S,6R,6aR)-6-(3,5-dimethoxyphenyl)-2,2-dimethyl-tetrahydro-3aH-cyclopenta[d][1,3]dioxol-4-ol 249d (0.67 g, 2.3 mmol, 1.3 eq), 2,4-dichloro-7H-pyrrolo[2,3-d]pyrimidine (0.33 g, 1.8 mmol, 1.0 eq), DIAD (0.71 g, 3.5 mmol, 2.0 eq) and PPh₃ (0.92 g, 3.5 mmol, 2.0 eq) in THF (8.0 mL) at 0° C. was stirred for 12 h to room temperature, then concentrated. The residue was dissolved in DCM (2 mL) and was purified by silica gel column chromatography (1:1 Petroleum ether: AcOEt) to afford 7-[(3aS,4R,6R,6aR)-6-(3,5-dimethoxyphenyl)-2,2-dimethyl-tetrahydro-3aH-cyclopenta[d][1,3]dioxol-4-yl]-2,4-dichloropyrrolo[2,3-d]pyrimidine 250d (0.42 g, 52%) as a white solid. LCMS (conditions XBridge C18, 50*3.0 mm, 3.5 μm, Mobile Phase A: Water/5 mM NH₄HCO₃, Mobile Phase B: Acetonitrile, 10-95% B-3 min, 1.50 mL/min): T_{R}=2.31 min; ES m/z [M+H]⁺: 464, 466.

### 7-[(3aS,4R,6R,6aR)-6-(3,5-Dimethoxyphenyl)-2,2-dimethyl-tetrahydro-3aH-cyclopenta[d][1,3]dioxol-4-yl]-2-chloropyrrolo[2,3-d]pyrimidin-4-amine (251d)

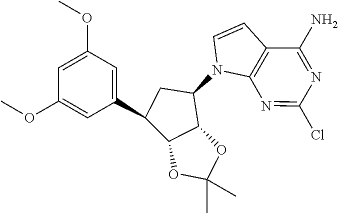 [SMILES: COc1cc(OC)cc([C@H]2C[C@@H](n3ccc4c(N)nc(Cl)nc43)[C@@H]3OC(C)(C)O[C@@H]32)c1]

Into a 40 mL vial were added 7-[(3aS,4R,6R,6aR)-6-(3,5-dimethoxyphenyl)-2,2-dimethyl-tetrahydro-3aH-cyclopenta[d][1,3]dioxol-4-yl]-2,4-dichloropyrrolo[2,3-d]pyrimidine 250d (0.4 g, 0.9 mmol, 1.0 eq) and 7M NH₃ in MeOH (4.0 mL) at room temperature. The reaction was stirred for 8 h at 80° C., then concentrated. The residue was dissolved in DCM (2 mL) and was purified by silica gel column chromatography (1:1 Petroleum ether: AcOEt) to afford 7-[(3aS,4R,6R,6aR)-6-(3,5-dimethoxyphenyl)-2,2-dimethyl-tetrahydro-3aH-cyclopenta[d][1,3]dioxol-4-yl]-2-chloropyrrolo[2,3-d]pyrimidin-4-amine 251d (0.13 g, 33%) as a white solid. LCMS (conditions XBridge C18, 50*3.0 mm, 3.5 μm, Mobile Phase A: Water/5 mM NH₄HCO₃, Mobile Phase B: Acetonitrile, 10-95% B-3 min, 1.50 mL/min): T_{R}=1.63 min; ES m/z [M+H]⁺: 445, 447.

### (1R,2S,3R,5R)-3-{4-Amino-2-chloropyrrolo[2,3-d]pyrimidin-7-yl]-5-(3,5-dimethoxyphenyl)cyclopentane-1,2-diol (664)

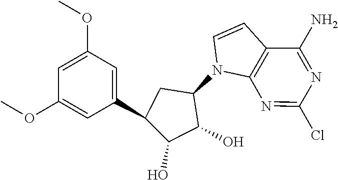 [SMILES: COc1cc(OC)cc([C@H]2C[C@@H](n3ccc4c(N)nc(Cl)nc43)[C@H](O)[C@@H]2O)c1]

Into an 8 mL vial were added 7-[(3aS,4R,6R,6aR)-6-(3,5-dimethoxyphenyl)-2,2-dimethyl-tetrahydro-3aH-cyclopenta[d][1,3]dioxol-4-yl]-2-chloropyrrolo[2,3-d]pyrimidin-4-amine 251d (85 mg, 0.2 mmol, 1.0 eq) and 4M HCl in MeOH (1.0 mL) at room temperature. The reaction was stirred for 0.5 h, then concentrated. The residue was dissolved in MeOH (3 mL) and was purified by Prep-HPLC [Column: Welch Ultimate XB-C18, 50*250 cm, 10 μm; Mobile Phase A: 0.1% HCl, Mobile Phase B: Acetonitrile; Flow rate: 90 mL/min; Gradient: 5% B-2 min, 5-33% B-12 min); Detector, UV 220&254 nm]to afford (1R,2S,3R,5R)-3-{4-amino-2-chloropyrrolo[2,3-d]pyrimidin-7-yl}-5-(3,5-dimethoxyphenyl)cyclopentane-1,2-diol HCl salt 664 (19.0 mg, 25%) as an off-white solid. LCMS (conditions: ZORBAX SB-Aq, 50*4.6 mm, 1.8 μm, Mobile Phase A: Water/0.02% TFA, Mobile Phase B: Acetonitrile, 5-95% B-5 min. 1.50 mL/min): T_{R}=2.28 min; ES m/z [M+H]⁺: 405, 407. ¹H NMR (400 MHZ, DMSO-d₆+D₂O) δ 7.48-7.40 (m, 1H), 6.70-6.61 (m, 1H), 6.54 (d, J=2.2 Hz, 2H), 6.36 (t, J=2.2 Hz, 1H), 4.85-4.74 (m, 1H), 4.26-4.18 (m, 1H), 4.05 (t, J=6.7 Hz, 1H), 3.75 (s, 6H), 3.08-2.97 (m, 1H), 2.37-2.25 (m, 1H), 2.02-1.89 (m, 1H).

### Synthesis of (1R,2S,3R,5R)-3-(4-amino-2-chloro-7H-pyrrolo[2,3-d]pyrimidin-7-yl)-5-(3-(aminomethyl)-5-methoxyphenyl)cyclopentane-1,2-diol (665)

### tert-butyl (3-methoxy-5-(4,4,5,5-tetramethyl-1,3,2-dioxaborolan-2-yl)benzyl)carbamate (282d)

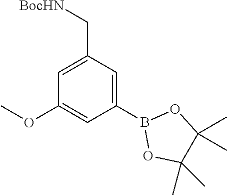

A solution of tert-butyl N-[(3-bromo-5-methoxyphenyl)methyl]carbamate (1.2 g, 3.795 mmol, 1.0 equiv) [WO20190374526], KOAc (744.9 mg, 7.59 mmol, 2.0 equiv) and 4,4,5,5-tetramethyl-2-(tetramethyl-1,3,2-dioxaborolan-2-yl)-1,3,2-dioxaborolane (1445.60 mg, 5.69 mmol, 1.5 equiv), Pd (PPh₃) 2Cl₂ (266.3 mg, 0.38 mmol, 0.1 equiv) in 1,4-dioxane (12.0 mL) was stirred for 3 h at 80° C. under nitrogen atmosphere. The resulting mixture was concentrated under reduced pressure. The resulting mixture was diluted with DCM (6.0 mL). The residue was purified by silica gel column chromatography (4:1 Petroleum ether/THF) to afford tert-butyl N-{[3-methoxy-5-(4,4,5,5-tetramethyl-1,3,2-dioxaborolan-2-yl)phenyl]methyl}carbamate 282d (820.0 mg, 59%) as a white solid. LCMS (conditions L-column3 ODS, 50*3.0 mm, 3.0 μm, Mobile Phase A: Water (0.05% ammonia water), Mobile Phase B: Acetonitrile, 5%-100% B-2 min): T_{R}=1.42 min; [M+H]⁺: 364.

### tert-butyl (3-((3aR,4R,6aR)-2,2-dimethyl-6-oxotetrahydro-4H-cyclopenta[d][1,3]dioxol-4-yl)-5-methoxybenzyl)carbamate (283d)

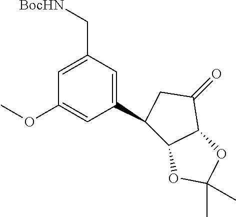

A solution of tert-butyl N-{[3-methoxy-5-(4,4,5,5-tetramethyl-1,3,2-dioxaborolan-2-yl)phenyl]methyl}carbamate 282 (1.20 g, 3.3 mmol, 1.1 equiv), (3aR,6aR)-2,2-dimethyl-3aH,6aH-cyclopenta[d][1,3]dioxol-4-one (464.0 mg, 3.0 mmol, 1.0 equiv) and Chloro(1,5-cyclooctadiene) rhodium (I) dimer (148.4 mg, 0.3 mmol, 0.1 equiv), 1M KOH (194 μL, 0.06 equiv) in MeOH (5.0 mL) was stirred for 10 min at room temperature under nitrogen atmosphere. The final reaction mixture was irradiated with microwave radiation for 2 h at 40° C. The resulting mixture was concentrated under reduced pressure. The residue was purified by silica gel column chromatography (4:1 Petroleum ether/THF) to afford tert-butyl N-({3-[(3aR,4R,6aR)-2,2-dimethyl-6-oxo-tetrahydrocyclopenta[d][1,3]dioxol-4-yl]-5-methoxyphenyl}methyl)carbamate 283d (750 mg, 63%) as a white solid. LCMS (conditions L-column3 ODS, 50*3.0 mm, 3.0 μm, Mobile Phase A: Water (0.05% ammonia water), Mobile Phase B: Acetonitrile, 5%-100% B-2 min): T_{R}=1.41 min; [M+H]⁺: 392.

### tert-butyl (3-((3aR,4R,6S,6aS)-6-hydroxy-2,2-dimethyltetrahydro-4H-cyclopenta[d][1,3]dioxol-4-yl)-5-methoxybenzyl)carbamate (284d)

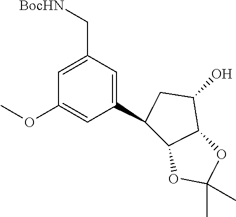

Into a 40 mL vial were added tert-butyl N-({3-[(3aR,4R,6aR)-2,2-dimethyl-6-oxo-tetrahydrocyclopenta[d][1,3]dioxol-4-yl]-5-methoxyphenyl}methyl)carbamate 283 (750.0 mg, 1.9 mmol, 1.0 equiv) and MeOH (8.0 mL) at room temperature. To the above mixture was added NaBH₄ (168.1 mg, 4.4 mmol, 2.3 equiv) in portions over 3 min at 0° C. The resulting mixture was stirred for 0.5 h at room temperature. The resulting mixture was concentrated under reduced pressure. The resulting mixture was diluted with DCM (5.0 mL) and was purified by silica gel column chromatography (2:1 Petroleum ether/THF) to afford tert-butyl N-({3-[(3aR,4R,6S,6aS)-6-hydroxy-2,2-dimethyl-tetrahydro-3aH-cyclopenta[d][1,3]dioxol-4-yl]-5-methoxyphenyl}methyl)carbamate 284 (500 0 mg, 66%) as a white solid. LCMS (conditions L-column3 ODS, 50*3.0 mm, 3.0 μm, Mobile Phase A: Water (0.05% ammonia water), Mobile Phase B: Acetonitrile, 5%-100% B-2 min): T_{R}=2.11 min; [M+H]⁺: 394.

### tert-butyl (3-((3aR,4R,6R,6aS)-6-(2,4-dichloro-7H-pyrrolo[2,3-d]pyrimidin-7-yl)-2,2-dimethyltetrahydro-4H-cyclopenta[d][1.3]dioxol-4-yl)-5-methoxybenzyl)carbamate (285d)

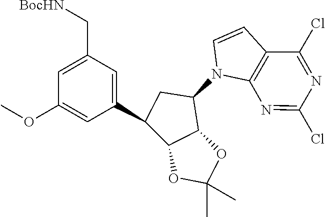 [SMILES: COc1cc(CNC(=O)OC(C)(C)C)cc([C@H]2C[C@@H](n3ccc4c(Cl)nc(Cl)nc43)[C@@H]3OC(C)(C)O[C@@H]32)c1]

A solution of tert-butyl N-({3-[(3aR,4R,6S,6aS)-6-hydroxy-2,2-dimethyl-tetrahydro-3aH-cyclopenta[d][1,3]dioxol-4-yl]-5-methoxyphenyl}methyl)carbamate 284d (482.2 mg, 1.2 mmol, 1.2 equiv), 2,4-dichloro-7H-pyrrolo[2,3-d]pyrimidine (192.0 mg, 1.0 mmol, 1.0 equiv) and PPh₃ (535.7 mg, 2.0 mmol, 2.0 equiv) in toluene (5.0 mL) was stirred for 5 min at room temperature under nitrogen atmosphere. To the above mixture was added DBAD (470.3 mg, 2.0 mmol, 2.0 equiv) in toluene (3.0 mL) dropwise over 10 min at 0° C. The resulting mixture was stirred for additional 3 h at 60° C. The resulting mixture was concentrated under reduced pressure. The resulting mixture was diluted with DCM (2.5 mL) and was purified by silica gel column chromatography (2:1 Petroleum ether/THF) to afford tert-butyl N-({3-[(3aR,4R,6R,6aS)-6-{2,4-dichloropyrrolo[2,3-d]pyrimidin-7-yl}-2,2-dimethyl-tetrahydro-3aH-cyclopenta[d][1,3]dioxol-4-yl]-5-methoxyphenyl}methyl)carbamate 285d (190 mg, 30%) as a white solid. LCMS (conditions L-column3 ODS, 50*3.0 mm, 3.0 μm, Mobile Phase A: Water (0.05% ammonia water), Mobile Phase B: Acetonitrile, 5%-95% B-2 min): T_{R}=1.43 min; [M+H]⁺: 563, 565.

### tert-butyl (3-((3aR,4R,6R,6aS)-6-(2-chloro-4-((4-methoxybenzyl)amino)-7H-pyrrolo[2,3-d]pyrimidin-7-yl)-2,2-dimethyltetrahydro-4H-cyclopenta[d][1,3]dioxol-4-yl)-5-methoxybenzyl)carbamate (286d)

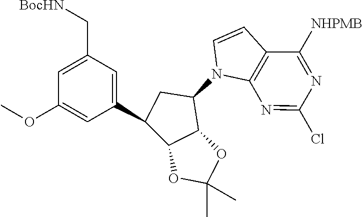

Into a 40 mL vial were added tert-butyl N-({3-[(3aR,4R,6R,6aS)-6-{2,4-dichloropyrrolo[2,3-d]pyrimidin-7-yl}-2,2-dimethyl-tetrahydro-3aH-cyclopenta[d][1,3]dioxol-4-yl]-5-methoxyphenyl}methyl)carbamate 285d (190.0 mg, 0.3 mmol, 1.0 equiv), (4-methoxyphenyl)methanamine (94.9 mg, 0.7 mmol, 3.0 equiv) and TEA (68.2 mg, 0.6 mmol, 2.0 equiv), EtOH (2.0 mL) at room temperature. The resulting mixture was stirred for 2 h at 80° C. The resulting mixture was concentrated under reduced pressure. The resulting mixture was diluted with DCM (1.0 mL). The residue was purified by silica gel column chromatography (4:1 Petroleum ether/THF) to afford tert-butyl N-({3-[(3aR,4R,6R,6aS)-6-(2-chloro-4-{[(4-methoxyphenyl)methyl]amino}pyrrolo[2,3-d]pyrimidin-7-yl)-2,2-dimethyl-tetrahydro-3aH-cyclopenta[d][1,3]dioxol-4-yl]-5-methoxyphenyl}methyl)carbamate 286d (140 mg, 63%) as a white solid. LCMS (conditions L-column3 ODS, 50*3.0 mm, 3.0 μm, Mobile Phase A: Water (0.05% ammonia water), Mobile Phase B: Acetonitrile, 5%-100% B-2 min): T_{R}=1.51 min; [M+H]⁺: 664, 666

### (1R,2S,3R,5R)-3-(4-amino-2-chloro-7H-pyrrolo[2,3-d]pyrimidin-7-yl)-5-(3-(aminomethyl)-5-methoxyphenyl)cyclopentane-1,2-diol (665)

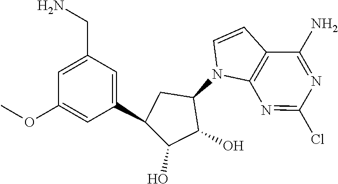 [SMILES: COc1cc(CN)cc([C@H]2C[C@@H](n3ccc4c(N)nc(Cl)nc43)[C@H](O)[C@@H]2O)c1]

Into a 8 mL vial were added tert-butyl N-({3-[(3′aR,4R,6R,6aS)-6-(2-chloro-4-{[(4-methoxyphenyl)methyl]amino}pyrrolo[2,3-d]pyrimidin-7-yl)-2,2-dimethyl-tetrahydro-3aH-cyclopenta[d][1,3]dioxol-4-yl]-5-methoxyphenyl}methyl)carbamate 286d (140.0 mg, 0.2 mmol, 1.0 equiv) and TFA (1.5 mL) at room temperature. The resulting mixture was stirred for 0.5h at 60° C. The reaction was monitored by LCMS. After completion of reaction, The resulting mixture was concentrated under reduced pressure and was basified to pH>8 with NH₃·H₂O. The residue was purified by Prep-HPLC with the following conditions (Column: Column: YMC-Actus Triart C18, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 20% B to 55% B in 7 min) to afford (1R,2S,3R,5R)-3-{4-amino-2-chloropyrrolo[2,3-d]pyrimidin-7-yl}-5-[3-(aminomethyl)-5-methoxyphenyl]cyclopentane-1,2-diol 665 (15.8 mg, 26%) as an off-white solid. LCMS (conditions ZORBAX SB-Aq, 50*4.6 mm, 1.8 μm, Mobile Phase A: Water/0.05% TFA, Mobile Phase B: Acetonitrile/0.05% TFA, 5%-(30%-60%)-95% B-5 min): T_{R}=1.07 min; [M+H]⁺: 404, 406.1H NMR (400 MHZ, DMSO-d₆+D₂O) δ 7.40 (d, J=3.6 Hz, 1H), 6.91 (s, 1H), 6.87-6.75 (m, 2H), 6.60 (d, J=3.6 Hz, 1H), 4.87-4.76 (m, 1H), 4.24 (t, J=6.8 Hz, 1H), 4.03 (t, J=6.6 Hz, 1H), 3.76 (s, 3H), 3.68 (s, 2H), 3.09-2.99 (m, 1H), 2.44-2.05 (m, 1H), 1.96 (q, J=11.6 Hz, 1H).

### Synthesis of (1R,2S,3R,5R)-3-(4-amino-2-chloro-7H-pyrrolo[2,3-d]pyrimidin-7-yl)-5-(3-bromo-5-methoxyphenyl)cyclopentane-1,2-diol (666)

### (3aR,6R,6aR)-6-(3-bromo-5-methoxyphenyl)-2,2-dimethyltetrahydro-4H-cyclopenta[d][1,3]dioxol-4-one (287d)

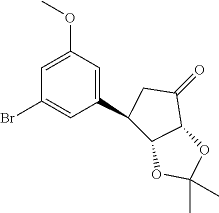 [SMILES: COc1cc(Br)cc([C@H]2CC(=O)[C@@H]3OC(C)(C)O[C@H]23)c1]

A solution of (3aR,6aR)-2,2-dimethyl-3aH,6aH-cyclopenta[d][1,3]dioxol-4-one (800 0 mg, 5.2 mmol, 1.0 equiv), 3-bromo-5-methoxycyclohexylboronic acid (1.84 g, 7.8 mmol, 1.5 equiv) and Chloro(1,5-cyclooctadiene) rhodium (I) dimer (255.8 mg, 0.5 mmol, 0.1 equiv), KOH (336 μL, 5.9 mmol, 1.1 equiv) in MeOH (8.0 mL) was stirred for 2 h at 40° C. under nitrogen atmosphere. The resulting mixture was concentrated under reduced pressure. The resulting mixture was diluted with DCM (4.0 mL). The residue was purified by silica gel column chromatography (4:1 Petroleum ether/THF) to afford (3aR,6R,6aR)-6-(3-bromo-5-methoxyphenyl)-2,2-dimethyl-tetrahydrocyclopenta[d][1,3]dioxol-4-one 287d (1.50 g, 84%) as a white solid. LCMS (conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-2 min): T_{R}=1.52 min; [M+H]⁺: 341, 343.

### (3aS,4S,6R,6aR)-6-(3-bromo-5-methoxyphenyl)-2,2-dimethyltetrahydro-4H-cyclopenta[d][1,3]dioxol-4-ol (288)

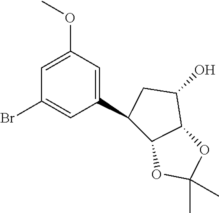 [SMILES: COc1cc(Br)cc([C@H]2C[C@H](O)[C@@H]3OC(C)(C)O[C@@H]32)c1]

Into a 40 mL vial were added (3aR,6R,6aR)-6-(3-bromo-5-methoxyphenyl)-2,2-dimethyl-tetrahydrocyclopenta[d][1,3]dioxol-4-one 287d (1.5 g, 4.3 mmol, 1.0 equiv) and NaBH₄ (249.4 mg, 6.6 mmol, 1.5 equiv) at 0° C. The resulting mixture was stirred for 0.5 h at room temperature. The reaction was quenched by the addition of Water/Ice (2.0 mL) at 0° C. The resulting mixture was extracted with EtOAc (3×50 mL). The combined organic layers were washed with brine, dried over anhydrous Na₂SO₄. After filtration, the filtrate was concentrated under reduced pressure. The residue was purified by silica gel column chromatography (4:1 Petroleum ether/THF) to afford (3aS,45,6R,6aR)-6-(3-bromo-5-methoxyphenyl)-2,2-dimethyl-tetrahydro-3aH-cyclopenta[d][1,3]dioxol-4-ol 288d (1 g, 66%) as a white solid. LCMS (conditions L-column3 ODS, 50*3.0 mm, 3.0 μm, Mobile Phase A: Water (0.05% ammonia water) Mobile Phase B: Acetonitrile, 5%-100% B-2 min): T_{R}=1.30 min; [M+H]⁺: 343, 345.

### 7-((3aS,4R,6R,6aR)-6-(3-bromo-5-methoxyphenyl)-2,2-dimethyltetrahydro-4H-cyclopenta[d][1,3]dioxol-4-yl)-2,4-dichloro-7H-pyrrolo[2,3-d]pyrimidine (289d)

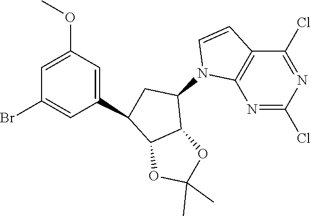 [SMILES: COc1cc(Br)cc([C@H]2C[C@@H](n3ccc4c(Cl)nc(Cl)nc43)[C@@H]3OC(C)(C)O[C@@H]32)c1]

A solution of 2,4-dichloro-7H-pyrrolo[2,3-d]pyrimidine (420.0 mg, 2.2 mmol, 1.0 equiv), (3aS,4S,6R,6aR)-6-(3-bromo-5-methoxyphenyl)-2,2-dimethyl-tetrahydro-3aH-cyclopenta[d][1,3]dioxol-4-ol 288d (1.0 g, 2.9 mmol, 1.3 equiv) and PPh₃ (1.2 g, 4.4 mmol, 2.0 equiv) in toluene (5.0 mL) was stirred for 5 min at room temperature under nitrogen atmosphere. To the above mixture was added DBAD (1.03 g, 4.5 mol, 2.0q) in toluene (5.0 mL) dropwise over 10 min at 0° C. The resulting mixture was stirred for 3 h at 60° C. The resulting mixture was concentrated under reduced pressure. The resulting mixture was diluted with DCM (10.0 mL). The residue was purified by silica gel column chromatography (4:1 Petroleum ether/THF) to afford 7-[(3aS,4R,6R,6aR)-6-(3-bromo-5-methoxyphenyl)-2,2-dimethyl-tetrahydro-3aH-cyclopenta[d][1,3]dioxol-4-yl]-2,4-dichloropyrrolo[2,3-d]pyrimidine 289d (1.2 g, 95%) as a white solid. LCMS (conditions L-column3 ODS, 50*3.0 mm, 3.0 μm, Mobile Phase A: Water/5 mM NH₄HCO₃+0.05% Ammonia Mobile Phase B: Acetonitrile/5% water 5%-100% B-2 min): T_{R}=1.77 min; [M+H]⁺: 512, 514.

### 7-((3aS,4R,6R,6aR)-6-(3-bromo-5-methoxyphenyl)-2,2-dimethyltetrahydro-4H-cyclopenta[d][1,3]dioxol-4-yl)-2-chloro-N-(4-methoxybenzyl)-7H-pyrrolo[2,3-d]pyrimidin-4-amine (290d)

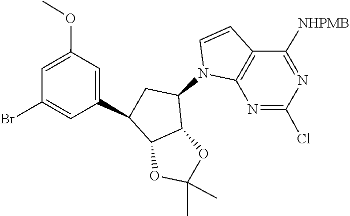

Into a 40*2 mL vial were added 7-[(3aS,4R,6R,6aR)-6-(3-bromo-5-methoxyphenyl)-2,2-dimethyl-tetrahydro-3aH-cyclopenta[d][1,3]dioxol-4-yl]-2,4-dichloropyrrolo[2,3-d]pyrimidine 289d (1.2 g, 2.4 mmol, 1.0 equiv), (4-methoxyphenyl)methanamine (465.1 mg, 3.3 mmol, 3.0 equiv) and EtOH (5.0 mL) at room temperature. The resulting mixture was stirred for 2 h at 80° C. The resulting mixture was concentrated under reduced pressure. The residue was purified by silica gel column chromatography (2:1 Petroleum ether/THF) to afford 7-[(3aS,4R,6R,6aR)-6-(3-bromo-5-methoxyphenyl)-2,2-dimethyl-tetrahydro-3aH-cyclopenta[d][1,3]dioxol-4-yl]-2-chloro-N-[(4-methoxyphenyl)methyl]pyrrolo[2,3-d]pyrimidin-4-amine 290d (450.0 mg, 31%) as an off-white solid. LCMS (conditions L-column3 ODS, 50*3.0 mm, 3.0 μm, Mobile Phase A: Water/5 mM NH₄HCO₃+0.05% Ammonia Mobile Phase B: Acetonitrile/5% water, 5%-100% B-2 min): T_{R}=1.74 min; [M+H]⁺: 613, 615.

### (1R,2S,3R,5R)-3-(4-amino-2-chloro-7H-pyrrolo[2,3-d]pyrimidin-7-yl)-5-(3-bromo-5-methoxyphenyl)cyclopentane-1,2-diol (666)

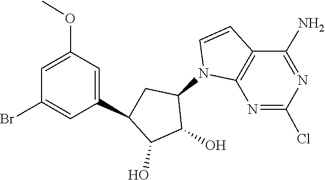 [SMILES: COc1cc(Br)cc([C@H]2C[C@@H](n3ccc4c(N)nc(Cl)nc43)[C@H](O)[C@@H]2O)c1]

Into a 8 mL vial were added 7-[(3aS,4R,6R,6aR)-6-(3-bromo-5-methoxyphenyl)-2,2-dimethyl-tetrahydro-3aH-cyclopenta[d][1,3]dioxol-4-yl]-2-chloro-N-[(4-methoxyphenyl)methyl]pyrrolo[2,3-d]pyrimidin-4-amine 290d (110 mg, 0.2 mmol, 1.0 equiv) and TFA (1.5 mL) at room temperature. The resulting mixture was stirred for 0.5h at 60° C. The reaction was monitored by LCMS. After completion of reaction, The resulting mixture was concentrated under reduced pressure and was basified to pH>8 with NH₃·H₂O. The residue was purified by Prep-HPLC with the following conditions (Column: Column: YMC-Actus Triart C18, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 20% B to 55% B in 7 min) to afford (1R,2S,3R,5R)-3-{4-amino-2-chloropyrrolo[2,3-d]pyrimidin-7-yl}-5-(3-bromo-5-methoxyphenyl)cyclopentane-1,2-diol 666 (17.1 mg, 21%) as an off-white solid. LCMS (conditions ZORBAX SB-Aq, 50*4.6 mm, 1.8 μm, Mobile Phase A: Water/0.05% TFA, Mobile Phase B: Acetonitrile/0.05% TFA, 5%-(30%-60%)-95% B-5 min): T_{R}=2.62 min; [M+H]⁺: 453, 455.1H NMR (400 MHZ, DMSO-d₆) δ 7.45 (s, 2H), 7.42 (d, J=3.6 Hz, 1H), 7.20 (s, 1H), 7.02 (t, J=2.1 Hz, 1H), 6.96 (s, 1H), 6.59 (d, J=3.6 Hz, 1H), 4.97 (br, s, 2H), 4.84-4.70 (m, 1H), 4.19 (t, J=6.5 Hz, 1H), 4.07 (t, J=7.0 Hz, 1H), 3.79 (s, 3H), 3.12-3.01 (m, 1H), 2.36-2.27 (m, 1H), 1.96 (q, J=11.9 Hz, 1H).

### Synthesis of (1R,2S,3R,5R)-3-(4-amino-2-chloro-7H-pyrrolo[2,3-d]pyrimidin-7-yl)-5-(3-(isothiazol-4-yl)-5-methoxyphenyl)cyclopentane-1,2-diol (667)

### 2-chloro-7-((3aS,4R,6R,6aR)-6-(3-(isothiazol-4-yl)-5-methoxyphenyl)-2,2-dimethyltetrahydro-4H-cyclopenta[d][1,3]dioxol-4-yl)-N-(4-methnxvhenzyl)-7H-nvrrolol2 3-dInvrimidin-4-amine (291d)

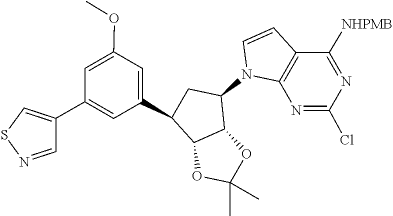

7-((3aS,4R,6R,6aR)-6-(3-bromo-5-methoxyphenyl)-2,2-dimethyltetrahydro-4H-cyclopenta[d][1,3]dioxol-4-yl)-2-chloro-N-(4-methoxybenzyl)-7H-pyrrolo[2,3-d]pyrimidin-4-amine 290d (180.0 mg, 0.3 mmol, 1.0 equiv), 4-(4,4,5,5-tetramethyl-1,3,2-dioxaborolan-2-yl)-1,2-thiazole (247.5 mg, 1.2 mmol, 4.0 equiv) and K₂CO₃ (121.5 mg, 0.8 mmol, 3.0 equiv), Pd (dppf) Cl₂ (10.7 mg, 0.1 mmol, 0.05 equiv) were added ino 1,4-dioxane/H₂O (5.0 mL/0.5 mL) under nitrogen atmosphere. The resulting mixture was stirred for 0.5h at 60° C. The resulting mixture was cooled to room temperature and added 10 mL H₂O. The resulting mixture was extracted with EtOAc (3×15 mL). The combined organic layers were washed with brine, dried over anhydrous Na₂SO₄. After filtration, the filtrate was concentrated under reduced pressure. The residue was purified by silica gel column chromatography (2:1 Petroleum ether/THF) to afford 7-[(3aS,4R,6R,6aR)-6-[3-methoxy-5-(1,2-thiazol-4-yl)phenyl]-2,2-dimethyl-tetrahydro-3aH-cyclopenta[d][1,3]dioxol-4-yl]-2-chloro-N-[(4-methoxyphenyl)methyl]pyrrolo[2,3-d]pyrimidin-4-amine 291d (100.0 mg, 55%) as a white solid. LCMS (conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-2 min): T_{R}=1.66 min; [M+H]⁺: 618, 620.

### (1R,2S,3R,5R)-3-(4-amino-2-chloro-7H-pyrrolo[2,3-d]pyrimidin-7-yl)-5-(3-(isothiazol-4-yl)-5-methoxyphenyl)cyclopentane-1,2-diol (667)

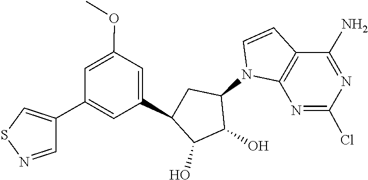 [SMILES: COc1cc(-c2cnsc2)cc([C@H]2C[C@@H](n3ccc4c(N)nc(Cl)nc43)[C@H](O)[C@@H]2O)c1]

Into a 8 mL vial were added 7-[(3aS,4R,6R,6aR)-6-[3-methoxy-5-(1,2-thiazol-4-yl)phenyl]-2,2-dimethyl-tetrahydro-3aH-cyclopenta[d][1,3]dioxol-4-yl]-2-chloro-N-[(4-methoxyphenyl)methyl]pyrrolo[2,3-d]pyrimidin-4-amine 291d (100.0 mg, 0.2 mmol, 1.0 equiv) and TFA (1.0 mL) at room temperature. The resulting mixture was stirred for 0.5h at 60° C. The reaction was monitored by LCMS. After completion of reaction, the resulting mixture was concentrated under reduced pressure and was basified to pH>8 with NH₃·H₂O. The residue was purified by Prep-HPLC with the following conditions (Column: Column: YMC-Actus Triart C18, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 20% B to 55% B in 7 min) to afford (1R,2S,3R,5R)-3-{4-amino-2-chloropyrrolo[2,3-d]pyrimidin-7-yl}-5-[3-methoxy-5-(1,2-thiazol-4-yl)phenyl]cyclopentane-1,2-diol 667 (19.1 mg, 25%) as an off-white solid. LCMS (conditions ZORBAX SB-Aq, 50*4.6 mm, 1.8 μm, Mobile Phase A: Water/0.05% TFA, Mobile Phase B: Acetonitrile/0.05% TFA, 5%-95% B-3 min): T_{R}=1.69 min; [M+H]⁺: 458, 460. ¹H NMR (400 MHZ, DMSO-d₆) δ 9.38 (s, 1H), 9.08 (s, 1H), 7.48-7.41 (m, 3H), 7.36 (s, 1H), 7.23 (t, J=2.0 Hz, 1H), 6.99-6.94 (m, 1H), 6.61 (d, J=3.5 Hz, 1H), 4.98-4.92 (m, 2H), 4.90-4.79 (m, 1H), 4.28-4.23 (m, 1H), 4.16-4.12 (m, 1H), 3.85 (s, 3H), 3.19-3.08 (m, 1H), 2.44-2.34 (m, 1H), 2.10-1.95 (m, 1H).

### Synthesis of (1R,2S,3R,5R)-3-{4-amino-2-chloropyrrolo[2,3-d]pyrimidin-7-yl}-5-[3-(trifluoromethoxy)phenyl]cyclopentane-1,2-dio (668)

### (3aR,6R,6aR)-2-methyl-6-[3-(trifluoromethoxy)phenyl]-tetrahydro-2H-cyclopenta[d][1,3]dioxol-4-one (292d)

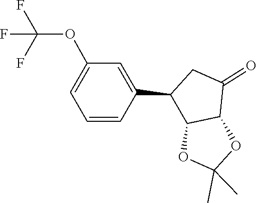 [SMILES: CC1(C)O[C@@H]2[C@@H](c3cccc(OC(F)(F)F)c3)CC(=O)[C@@H]2O1]

A solution of (3aR,6aR)-2,2-dimethyl-3aH,6aH-cyclopenta[d][1,3]dioxol-4-one (500.0 mg, 3.2 mmol, 1.0 equiv) in MeOH was treated with 3-(trifluoromethoxy)phenylboronic acid (868.2 mg, 4.2 mmol, 1.3 equiv) and [RhCl (COD)]2 (79.9 mg, 0.1 mmol, 0.1 equiv) at room temperature under nitrogen atmosphere followed by the addition of KOH (210 μL, 0.4 mmol, 1.5 equiv) in portions at room temperature. The final reaction mixture was irradiated with microwave radiation for 3 h at 40° C. The reaction was monitored by LCMS. The resulting mixture was concentrated under reduced pressure. The residue was purified by silica gel column chromatography (5:1 Petroleum ether/ethyl acetate) to afford (3aR,6R,6aR)-2-methyl-6-[3-(trifluoromethoxy)phenyl]-tetrahydro-2H-cyclopenta[d][1,3]dioxol-4-one 292d (600.0 mg, 61%) as a yellow oil. LCMS (conditions L-column3 ODS, 50*3.0 mm, 3.0 μm, Mobile Phase A: Water (0.05% ammonia water, Mobile Phase B: Acetonitrile, 10%-95% B-2 min): T_{R}=1.52 min; [M−H]⁺: 317.

### (3aS,45,6R,6aR)-2,2-dimethyl-6-(3-(trifluoromethoxy)phenyl)tetrahydro-4H-cyclopenta[d][1,3]dioxol-4-ol (293d)

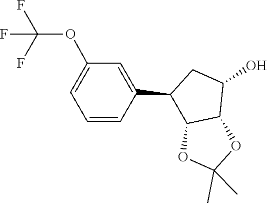 [SMILES: CC1(C)O[C@@H]2[C@H](O1)[C@@H](c1cccc(OC(F)(F)F)c1)C[C@@H]2O]

To a stirred solution of (3aR,6R,6aR)-2,2-dimethyl-6-[3-(trifluoromethoxy)phenyl]-tetrahydrocyclopenta[d][1,3]dioxol-4-one 292d (580.0 mg, 1.8 mmol, 1.0 equiv) in MeOH was added NaBH₄ (104.0 mg, 2.7 mmol, 1.5 equiv) in portions at 0° C. The resulting mixture was stirred for 30 min at room temperature. The reaction was monitored by LCMS. The reaction was quenched by the addition of Water/Ice at 0° C. The resulting mixture was extracted with EtOAc (3×20 mL). The combined organic layers were washed with brine (2×10 mL), dried over anhydrous Na₂SO₄. After filtration, the filtrate was concentrated under reduced pressure. The residue was purified by silica gel column chromatography (3:1 Petroleum ether/tetrahydrofuran) to afford (3aS,4S,6R,6aR)-2,2-dimethyl-6-[3-(trifluoromethoxy)phenyl]-tetrahydro-3aH-cyclopenta[d][1,3]dioxol-4-ol 293d (406.0 mg, 70%) as a colourless liquid. LCMS (conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-2 min): T_{R}=1.46 min; [M+H]⁺: 319.

### 2,4-dichloro-7-((3aS,4R,6R,6aR)-2,2-dimethyl-6-(3-(trifluoromethoxy)phenyl)tetrahydro-4H-cyclopenta[d][1,3]dioxol-4-yl)-7H-pyrrolo[2,3-d]pyrimidine (294d)

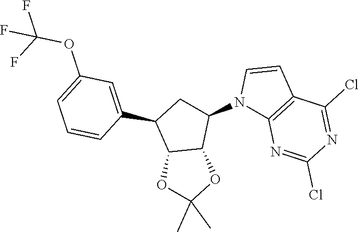 [SMILES: CC1(C)O[C@@H]2[C@H](O1)[C@@H](c1cccc(OC(F)(F)F)c1)C[C@H]2n1ccc2c(Cl)nc(Cl)nc21]

A solution of (3aS,4S,6R,6aR)-2,2-dimethyl-6-[3-(trifluoromethoxy)phenyl]-tetrahydro-3aH-cyclopenta[d][1,3]dioxol-4-ol 293d (392.3 mg, 2.4 mmol, 1.2 equiv) in THF was treated with 2,4-dichloro-7H-pyrrolo[2,3-d]pyrimidine (390.0 mq, 2.0 mmol, 1.0 equiv) and PPh₃ (1088.1 mg, 4.1 mmol, 2.0 equiv) at room temperature under nitrogen atmosphere followed by the addition of DIAD (838.9 mg, 4.1 mmol, 2.0 equiv) dropwise at room temperature. The resulting mixture was stirred for overnight at room temperature. The reaction was monitored by LCMS. The resulting mixture was concentrated under reduced pressure. The residue was dissolved in DCM (10 mL) and was purified by silica gel column chromatography (2:1 Petroleum ether/Tetrahydrofuran) to afford 7-[(3aS,4R,6R,6aR)-2,2-dimethyl-6-[3-(trifluoromethoxy)phenyl]-tetrahydro-3aH-cyclopenta[d][1,3]dioxol-4-yl]-2,4-dichloropyrrolo[2,3-d]pyrimidine 294d (340.0 mq, 34%) as an off-white solid. LCMS (conditions Proshell HPH-C18, 3.0*50 mm, 2.7 μm, Mobile Phase A: Water (0.05% ammonia water, Mobile Phase B: Acetonitrile, 5%-100% B-2 min): T_{R}=1.53 min; [M+H]⁺: 488, 490.

### 2-chloro-7-((3aS,4R,6R,6aR)-2,2-dimethyl-6-(3-(trifluoromethoxy)phenyl)tetrahydro-4H-cyclopenta[d][1,3]dioxol-4-yl)-N-(4-methoxybenzyl)-7H-pyrrolo [2,3-d]pyrimidin-4-amine (295d)

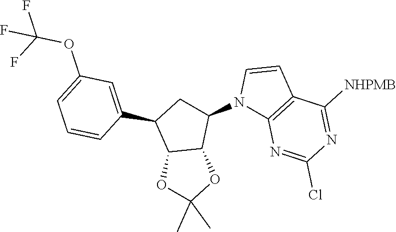

Into a 40 mL vial were added 7-[(3aS,4R,6R,6aR)-2,2-dimethyl-6-[3-(trifluoromethoxy)phenyl]-tetrahydro-3aH-cyclopenta[d][1,3]dioxol-4-yl]-2,4-dichloropyrrolo[2,3-d]pyrimidine 294d (320.0 mg, 0.6 mmol, 1.0 equiv), TEA (132.6 mg, 1.3 mmol, 2.0 equiv), EtOH (3.5 mL) and 1-(4-methoxyphenyl)methanamine (269.7 mg, 1.9 mmol, 3.0 equiv) at room temperature. The resulting mixture was stirred for 1 h at 80° C. The reaction was monitored by LCMS. The resulting mixture was concentrated under reduced pressure. The residue was purified by silica gel column chromatography (2:1 Petroleum ether/Tetrahydrofuran) to afford 2-chloro-7-((3aS,4R,6R,6aR)-2,2-dimethyl-6-(3-(trifluoromethoxy)phenyl)tetrahydro-4H-cyclopenta[d][1,3]dioxol-4-yl)-N-(4-methoxybenzyl)-7H-pyrrolo[2,3-d]pyrimidin-4-amine 295d (200.0 mg, 52%) as a colourless oil. LCMS (conditions L-column3 ODS, 50*3.0 mm, 3.0 μm, Mobile Phase A: Water (0.05% ammonia water, Mobile Phase B: Acetonitrile, 5%-100% B-2.0 min): T_{R}=1.59 min; [M+H]⁺: 589, 591.

### (1R,2S,3R,5R)-3-(4-amino-2-chloro-7H-pyrrolo[2,3-d]pyrimidin-7-yl)-5-(3-(trifluoromethoxy)phenyl)cyclopentane-1,2-diol (668)

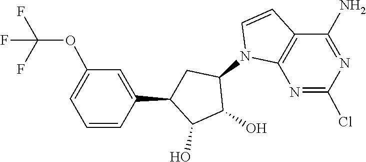 [SMILES: Nc1nc(Cl)nc2c1ccn2[C@@H]1C[C@H](c2cccc(OC(F)(F)F)c2)[C@@H](O)[C@H]1O]

Into an 8 mL vial were added 2-chloro-7-((3aS,4R,6R,6aR)-2,2-dimethyl-6-(3-(trifluoromethoxy)phenyl)tetrahydro-4H-cyclopenta[d][1,3]dioxol-4-yl)-N-(4-methoxybenzyl)-7H-pyrrolo[2,3-d]pyrimidin-4-amine 295d (190.0 mg, 0.3 mmol, 1.0 equiv) and trifluoroacetic acid (2.5 mL) at room temperature. The resulting mixture was stirred for 1 h at 60° C. The resulting mixture was concentrated under reduced pressure. The mixture was basified to pH>8 with NH₃·H₂O. The residue was was purified by Prep-HPLC with the following conditions (Column: YMC-Actus Triart C18 ExRS, 30*150 mm, 5 μm; Mobile Phase A: Water (0.05% NH₃·H₂O), Mobile Phase B: ACN; Flow rate: 60 mL/min; Gradient: 26% B to 56% B in 8 min, 56% B; Wave Length: 220 nm) to afford (1R,2S,3R,5R)-3-(4-amino-2-chloro-7H-pyrrolo[2,3-d]pyrimidin-7-yl)-5-(3-(trifluoromethoxy)phenyl)cyclopentane-1,2-diol 668 (36.2 mg, 26.17%) as a white solid. LCMS (conditions Cortecs C18+, 50*3.0 mm, 2.7 μm, Mobile Phase A: Water/0.05% TFA, Mobile Phase B: Acetonitrile/0.05% TFA, 5%-95% B-3 min): T_{R}=1.35 min; [M+H]⁺: 429, 431. ¹H NMR (400 MHZ, DMSO-d) δ 7.54-7.36 (m, 6H), 7.27-7.20 (m, 1H), 6.60 (d,)=3.5 Hz, 1H), 5.02 (s, 2H), 4.87-4.73 (m, 1H), 4.23 (t, J=6.5 Hz, 1H), 4.08 (t, J=6.9 Hz, 1H), 3.22-3.11 (m, 1H), 2.45-2.33 (m, 1H), 2.01 (q, J=11.8 Hz, 1H).

### Synthesis of (1R,2S,3R,5R)-3-(4-amino-5-bromo-2-chloro-7H-pyrrolo[2,3-d]pyrimidin-7-yl)-5-(3-methoxyphenyl)cyclopentane-1,2-diol (669)

### (3aR,6R,6aR)-6-(3-methoxyphenyl)-2,2-dimethyltetrahydro-4Hcyclopenta[d][1,3]dioxol-4-one (296d)

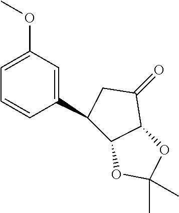 [SMILES: COc1cccc([C@H]2CC(=O)[C@@H]3OC(C)(C)O[C@H]23)c1]

Into a 40 mL vial were added mixture of (3aR,6aR)-2,2-dimethyl-3aH,6aH-cyclopenta[d][1,3]dioxol-4-one (2.0 g, 12.9 mmol, 1.0 equiv) and 3-methoxyphenylboronic acid (2.6 g, 16.9 mmol, 1.3 equiv) in dioxane (20.0 mL) were added acetylacetonatobis(ethylene) rhodium (I)(230 mg, 0.1 equiv) and R-BINAP (160 mg, 2.58 mmol, 0.02 equiv) in portions at room temperature under N₂ atmosphere The resulting mixture was stirred for 1 h at 100° C. The resulting mixture was concentrated under reduced pressure. The residue was purified by silica gel column chromatography (1:10 Tetrahydrofuran: Petroleum ether) to afford (3aR,6R,6aR)-6-(3-methoxyphenyl)-2,2-dimethyl-tetrahydrocyclopenta[d][1,3]dioxol-4-one 296d (3.1 g, 91%) as a yellow oil. LCMS (conditions XBridge C18, 50*3.0 mm, 3.5 μm, Mobile Phase A: Water/5 mM NH₄HCO₃, Mobile Phase B: Acetonitrile, 5%-100% B-3 min): T_{R}=1.83 min; [M+H]⁺: 263.

### (3aS,4S,6R,6aR)-6-(3-methoxyphenyl)-2,2-dimethyltetrahydro-4H-cyclopenta[d][1,3]dioxol-4-ol (297d)

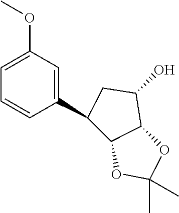 [SMILES: COc1cccc([C@H]2C[C@H](O)[C@@H]3OC(C)(C)O[C@@H]32)c1]

Into a 40 mL vial were added mixture of (3aR,6R,6aR)-6-(3-methoxyphenyl)-2,2-dimethyl-tetrahydrocyclopenta[d][1,3]dioxol-4-one 296d (3.1 g, 11.8 mmol, 1 equiv) in MeOH (31.0 mL,) was added NaBH₄ (670.6 mg, 17.7 mmol, 1.5 equiv) in portions at 0° C. The resulting mixture was stirred for 0.5 h at room temperature The reaction was quenched by the addition of NH₄Cl (5.0 mL) at 0° C. The resulting mixture was extracted with AcOEt (3×50 mL). The combined organic layers were washed with brine, dried with Na₂SO₄. After filtration, the filtrate was concentrated under reduced pressure. The residue was purified by silica gel column chromatography (1:5 Tetrahydrofuran/Petroleum ether) to afford (3aS,4S,6R,6aR)-6-(3-methoxyphenyl)-2,2-dimethyltetrahydro-4H-cyclopenta[d][1,3]dioxol-4-ol 297d (2.7 g, 86%) as a yellow oil. LCMS (conditions XBridge C18, 50*3.0 mm, 3.5 μm, Mobile Phase A: Water/5 mM NH₄HCO₃, Mobile Phase B: Acetonitrile, 5%-100% B-3 min): T_{R}=1.59 min; [M+H]⁺: 265.

### 5-bromo-2,4-dichloro-7-((3aS,4R,6R,6aR)-6-(3-methoxyphenyl)-2,2-dimethyltetrahydro-4H-cyclopenta[d][1,3]dioxol-4-yl)-7H-pyrrolo[2,3-d]pyrimidine (298d)

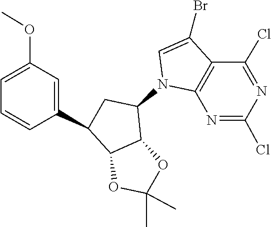 [SMILES: COc1cccc([C@H]2C[C@@H](n3cc(Br)c4c(Cl)nc(Cl)nc43)[C@@H]3OC(C)(C)O[C@@H]32)c1]

Into a 40 mL vial were added mixture of (3aS,4S,6R,6aR)-6-(3-methoxyphenyl)-2,2-dimethyl-tetrahydro-3aH-cyclopenta[d][1,3]dioxol-4-ol 297d (772.4 mg, 3.0 mmol, 1.3 equiv) and 5-bromo-2,4-dichloro-7H-pyrrolo[2,3-d]pyrimidine (600 mg, 2.2 mmol, 1.00 equiv) in toluene (12.0 mL) was added PPh₃ (1179.2 mg, 4.5 mmol, 2.0 equiv) in portions at room temperature under N₂ atmosphere. To the above mixture was added DBAD (1035.2 mg, 4.5 mmol, 2.0 equiv) in portions over 1 min at 0° C. The resulting mixture was stirred for 1 h at 50° C. The resulting mixture was concentrated under reduced pressure. The residue was purified by silica gel column chromatography (1:5 Tetrahydrofuran/Petroleum ether) to afford 5-bromo-2,4-dichloro-7-((3aS,4R,6R,6aR)-6-(3-methoxyphenyl)-2,2-dimethyltetrahydro-4H-cyclopenta[d][1,3]dioxol-4-yl)-7H-pyrrolo[2,3-d]pyrimidine 298d (700 mg, 61%) as a yellow oil. LCMS (conditions XBridge C18, 50*3.0 mm, 3.5 μm, Mobile Phase A: Water/5 mM NH₄HCO₃, Mobile Phase B: Acetonitrile, 5%-100% B-3 min): T_{R}=2.39 min; [M+H]⁺: 512, 514.

### 5-bromo-2-chloro-7-((3aS,4R,6R,6aR)-6-(3-methoxyphenyl)-2,2-dimethyltetrahydro-4H-cyclopenta[d][1,3]dioxol-4-yl)-7H-pyrrolo[2,3-d]pyrimidin-4-amine (299d)

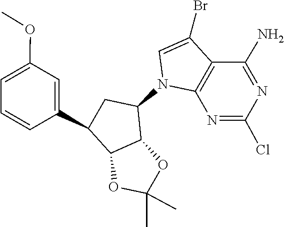 [SMILES: COc1cccc([C@H]2C[C@@H](n3cc(Br)c4c(N)nc(Cl)nc43)[C@@H]3OC(C)(C)O[C@@H]32)c1]

Into a 40 mL vial were added mixture of 5-bromo-2,4-dichloro-7-((3aS,4R,6R,6aR)-6-(3-methoxyphenyl)-2,2-dimethyltetrahydro-4H-cyclopenta[d][1,3]dioxol-4-yl)-7H-pyrrolo[2,3-d]pyrimidine 298d (100.0 mg, 0.2 mmol, 1.0 equiv) in 7M NH₃ in MeOH (2.0 mL) at room temperature. The resulting mixture was stirred for 1 h at 80° C. The resulting mixture was concentrated under reduced pressure to afford 5-bromo-2-chloro-7-((3aS,4R,6R,6aR)-6-(3-methoxyphenyl)-2,2-dimethyltetrahydro-4H-cyclopenta[d][1,3]dioxol-4-yl)-7H-pyrrolo[2,3-d]pyrimidin-4-amine 299d (80.0 mg, 83%) as a white solid. LCMS (conditions XBridge C18, 50*3.0 mm, 3.5 μm, Mobile Phase A: Water/5 mM NH₄HCO₃, Mobile Phase B: Acetonitrile, 5%-100% B-2 min): T_{R}=1.58 min; [M+H]⁺: 493, 495.

### (1R,2S,3R,5R)-3-(4-amino-5-bromo-2-chloro-7H-pyrrolo[2,3-d]pyrimidin-7-yl)-5-(3-methoxyphenyl)cyclopentane-1,2-diol (669)

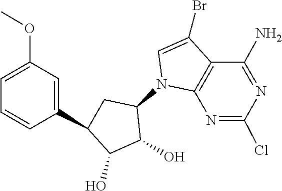 [SMILES: COc1cccc([C@H]2C[C@@H](n3cc(Br)c4c(N)nc(Cl)nc43)[C@H](O)[C@@H]2O)c1]

Into a 40 mL vial were added mixture of 5-bromo-2-chloro-7-((3aS,4R,6R,6aR)-6-(3-methoxyphenyl)-2,2-dimethyltetrahydro-4H-cyclopenta[d][1,3]dioxol-4-yl)-7H-pyrrolo[2,3-d]pyrimidin-4-amine 299d (80.0 mg, 0.14 mmol, 1.0 equiv) in TFA (1.0 mL) at room temperature. The resulting mixture was stirred for 1 h at 50° C. The resulting mixture was concentrated under reduced pressure. The mixture was basified to pH>8 with NH₃·H₂O and was purified by Prep-HPLC with the following conditions (Column: XBridge C18, 19*150 mm, 5 μm; Mobile Phase A: 20 mM NH₄HCO₃+0.05% NH₃·H₂O, Mobile Phase B: ACN; Flow rate: 20 mL/min; Gradient: 10% to 65% in 8 min) to afford (1R,2S,3R,5R)-3-(4-amino-5-bromo-2-chloro-7H-pyrrolo[2,3-d]pyrimidin-7-yl)-5-(3-methoxyphenyl)cyclopentane-1,2-diol 669 (9.9 mg, 14%) as an off-white solid. LCMS (conditions Cortecs C18+, 50*3.0 mm, 2.7 μm, Mobile Phase A: Water/0.05% TFA, Mobile Phase B: Acetonitrile/0.05% TFA, 5%-95% B-3 min): T_{R}=1.41 min; [M+H]⁺: 453, 455. ¹H NMR (400 MHZ, DMSO-d₆) δ 7.77 (s, 1H), 7.29-7.21 (m, 1H), 6.98 (d, J=7.4 Hz, 1H), 6.96-6.92 (m, 1H), 6.80 (dd, J=8.0, 2.6 Hz, 1H), 4.98 (d, J=6.2 Hz, 1H), 4.91 (d, J=6.0 Hz, 1H), 4.89-4.80 (m, 1H), 4.29-4.20 (m, 1H), 4.04-3.94 (m, 1H), 3.77 (s, 3H), 3.10-2.98 (m, 1H), 2.36-2.25 (m, 1H), 2.02-1.89 (m, 1H).

### (1R,2S,3R,5R)-3-{4-amino-2-chloropyrrolo[2,3-d]pyrimidin-7-yl}-5-(3-methoxyphenyl)cyclopentane-1,2-diol)(670)

### 7-[(3aS,4R,6R,6aR)-6-(3-methoxyphenyl)-2-methyl-hexahydrocyclopenta[d][1,3]dioxol-4-yl]-2,4-dichloropyrrolo[2,3-d]pyrimidine (300d)

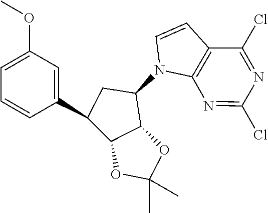 [SMILES: COc1cccc([C@H]2C[C@@H](n3ccc4c(Cl)nc(Cl)nc43)[C@@H]3OC(C)(C)O[C@@H]32)c1]

A solution of (3aS,4S,6R,6aR)-6-(3-methoxyphenyl)-2,2-dimethyl-tetrahydro-3aH-cyclopenta[d][1,3]dioxol-4-ol (105.4 mg, 0.3 mmol, 1.5 equiv) in THF was treated with 2,4-dichloro-7H-pyrrolo[2,3-d]pyrimidine (50.0 mg, 0.2 mmol, 1.0 equiv) and PPh₃ (139.5 mg, 0.5 mmol, 2.0 equiv) at room temperature followed by the addition of DIAD (107.5 mg, 0.5 mmol, 2.0 equiv) dropwise at 0° C. The resulting mixture was stirred for 3 h at room temperature. The reaction was monitored by LCMS. The resulting mixture was concentrated under reduced pressure. The residue was purified by silica gel column chromatography (2:1 petroleum ether/ethyl acetate) to afford 7-[(3aS,4R,6R,6aR)-6-(3-methoxyphenyl)-2-methyl-hexahydrocyclopenta[d][1,3]dioxol-4-yl]-2,4-dichloropyrrolo[2,3-d]pyrimidine 300d (110 mg, 63%) as a colourless oil. LCMS (conditions: L-column3 ODS, 50*3.0 mm, Mobile Phase A: Water/5 mM NH₄HCO₃+0.05% Ammonia, Mobile Phase B: Acetonitrile/5% water, 5%-100% B-2 min): T_{R}=1.69 min; [M+H]⁺: 434, 436.

### 7-[(3aS,4R,6R,6aR)-6-(3-methoxyphenyl)-2,2-dimethyl-tetrahydro-3aH-cyclopenta[d][1,3]dioxol-4-yl]-2-chloro-N-[(4-methoxyphenyl)methyl]pyrrolo[2,3-d]pyrimidin-4-amine)(301d)

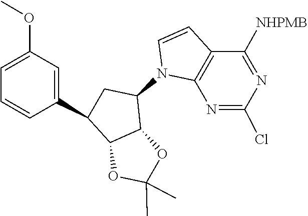

Into 8 mL vial were added 7-[(3aS,4R,6R,6aR)-6-(3-methoxyphenyl)-2,2-dimethyl-tetrahydro-3aH-cyclopenta[d][1,3]dioxol-4-yl]-2,4-dichloropyrrolo[2,3-d]pyrimidine (95.0 mq, 0.2 mmol, 1.0 equiv), EtOH (1.5 mL), and TEA (44.2 mg, 0.4 mmol, 2.0 equiv) at room temperature. To the above mixture was added 1-(4-methoxyphenyl)methanamine (60.0 mq, 0.4 mmol, 2.0 equiv) dropwise at room temperature. The resulting mixture was stirred for 2 h at 80° C. The reaction was monitored by LCMS. The resulting mixture was concentrated under reduced pressure. The residue was purified by silica gel column chromatography (3:1 Petroleum ether/Tetrahydrofuran) to afford 7-[(3aS,4R,6R,6aR)-6-(3-methoxyphenyl)-2,2-dimethyl-tetrahydro-3aH-cyclopenta[d][1,3]dioxol-4-yl]-2-chloro-N-[(4-methoxyphenyl)methyl]pyrrolo[2,3-d]pyrimidin-4-amine (65 mg, 56%) as a colourless oil. LCMS (conditions: L-column3 ODS, 50*3.0 mm, Mobile Phase A: Water (0.05% ammonia water), Mobile Phase B: Acetonitrile, 5%-95% B-3 min): T_{R}=2.15 min; [M+H]⁺: 535, 537.

### (1R,2S,3R,5R)-3-{4-amino-2-chloropyrrolo[2,3-d]pyrimidin-7-yl}-5-(3-methoxyphenyl)cyclopentane-1,2-diol) (670)

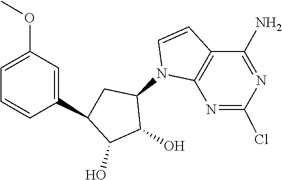 [SMILES: COc1cccc([C@H]2C[C@@H](n3ccc4c(N)nc(Cl)nc43)[C@H](O)[C@@H]2O)c1]

Into a 40 mL vial were added 7-[(3aS,4R,6R,6aR)-6-(3-methoxyphenyl)-2,2-dimethyl-tetrahydro-3aH-cyclopenta[d][1,3]dioxol-4-yl]-2-chloro-N-[(4-methoxyphenyl)methyl]pyrrolo[2,3-d]pyrimidin-4-amine (65.0 mq, 0.1 mmol, 1.0 equiv) and TFA (2.0 mL) at room temperature. The resulting mixture was stirred for 30 min at 60° C. The reaction was monitored by LCMS. The mixture was basified to Ph>8 with saturated NaHCO₃(aq.). The residue was purified by Prep-HPLC (Column: YMC-Actus Triart C18 ExRS, 30*150 mm, 5 μm; Mobile Phase A: 10mmolNH4HCO3+0.05% NH₃·H₂O, Mobile Phase B: ACN; Flow rate: 60 mL/min; Gradient: 18% B to 48% B in 8 min) to afford (1R,2S,3R,5R)-3-{4-amino-2-chloropyrrolo[2,3-d]pyrimidin-7-yl}-5-(3-methoxyphenyl)cyclopentane-1,2-diol 670 (11 mq, 24%) as a white solid. LCMS (conditions: ZORBAX SB-Aq, 50*4.6 mm, 1.8 μm, Mobile Phase A: Water/0.05% TFA, Mobile Phase B: Acetonitrile/0.05% TFA, 5%-95% B-3 min): T_{R}=1.44 min; [M+H]⁺: 375, 377. ¹H NMR (400 MHZ, DMSO-d₆) δ 7.45 (s, 2H), 7.40 (d, J=3.6 Hz, 1H), 7.25 (t, J=7.9 Hz, 1H), 6.99 (d, J=7.8 Hz, 1H), 6.94 (t, J=2.0 Hz, 1H), 6.80 (dd, J=8.2, 2.5 Hz, 1H), 6.59 (d, J=3.6 Hz, 1H), 4.93 (d, J=6.0 Hz, 1H), 4.89 (d, J=6.2 Hz, 1H), 4.86-4.76 (m, 1H), 4.24 (q, J=6.5 Hz, 1H), 4.05 (q, J=6.5 Hz, 1H), 3.77 (s, 3H), 3.12-3.01 (m, 1H), 2.40-2.28 (m, 1H), 1.97 (q, J=11.7 Hz, 1H).

### Synthesis of (1R,2S,3R,5R)-3-(4-amino-2-chloro-5-(1-methyl-1H-pyrazol-3-yl)-7H-pyrrolo[2,3-d]pyrimidin-7-yl)-5-(3-methoxyphenyl)cyclopentane-1,2-diol (671)

### 2-chloro-7-((3aS,4R,6R,6aR)-6-(3-methoxyphenyl)-2,2-dimethyltetrahydro-4H-cyclopenta[d][1,3]dioxol-4-yl)-5-(1-methyl-1H-pyrazol-3-yl)-7H-pyrrolo[2,3-d]pyrimidin-4-amine (299d)

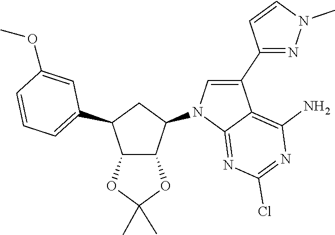

Into a 40 mL vial were added mixture of 7-[(3aS,4R,6R,6aR)-6-(3-methoxyphenyl)-2,2-dimethyl-tetrahydro-3aH-cyclopenta[d][1,3]dioxol-4-yl]-5-bromo-2-chloropyrrolo[2,3-d]pyrimidin-4-amine 299d (110.0 mg, 0.2 mmol, 1.0 equiv) and 1-methyl-3-(4,4,5,5-tetramethyl-1,3,2-dioxaborolan-2-yl)pyrazole (92.7 mg, 0.4 mmol, 2.0 equiv) in 1,4-dioxane (2.2 mL) and H₂O (0.22 mL) were added Pd (dtbpf) Cl₂ (14.5 mg, 0.02 mmol, 0.1 equiv) and K₃PO₄ (141.8 mg, 0.7 mmol, 3.0 equiv) in portions at room temperature under N₂ atmosphere. The resulting mixture was stirred for 0.5 h at 80° C. The resulting mixture was concentrated under reduced pressure. The residue was purified by silica gel column chromatography (1:2 tetrahydrofuran/Petroleum ether) to afford 7-[(3aS,4R,6R,6aR)-6-(3-methoxyphenyl)-2,2-dimethyl-tetrahydro-3aH-cyclopenta[d][1,3]dioxol-4-yl]-2-chloro-5-(1-methylpyrazol-3-yl)pyrrolo[2,3-d]pyrimidin-4-amine 302d (80.0 mg, 73%) as a yellow solid. LCMS (conditions XBridge C18, 50*3.0 mm, 3.5 μm, Mobile Phase A: Water/5 mM NH₄HCO₃, Mobile Phase B: Acetonitrile, 5%-100% B-2 min): T_{R}=1.56 min; [M+H]⁺: 495, 497.

### (1R,2S,3R,5R)-3-(4-amino-2-chloro-5-(1-methyl-1H-pyrazol-3-yl)-7H-pyrrolo[2,3-d]pyrimidin-7-yl)-5-(3-methoxyphenyl)cyclopentane-1,2-diol (671)

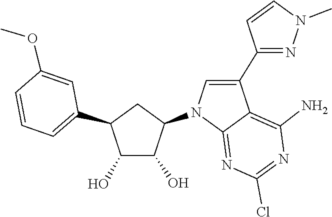

Into a 40 mL vial were added mixture of 7-[(3aS,4R,6R,6aR)-6-(3-methoxyphenyl)-2,2-dimethyl-tetrahydro-3aH-cyclopenta[d][1,3]dioxol-4-yl]-2-chloro-5-(1-methylpyrazol-3-yl)pyrrolo[2,3-d]pyrimidin-4-amine 302d (72.0 mg, 0.2 mmol, 1.0 equiv) in TFA (1.5 mL) at room temperature. The resulting mixture was stirred for 2 h at 50° C. The resulting mixture was concentrated under reduced pressure. The mixture was basified to pH>7 with NH₃·H₂O. The residue was purified by Prep-HPLC with the following conditions (Column: XBridge C18, 19*150 mm, 5 μm; Mobile Phase A: 20 mM NH₄HCO₃+0.05% NH₃·H₂O, Mobile Phase B: ACN; Flow rate: 20 mL/min; Gradient: 10% to 65% in 8 min) to afford (1R,2S,3R,5R)-3-[4-amino-2-chloro-5-(1-methylpyrazol-3-yl)pyrrolo[2,3-d]pyrimidin-7-yl]-5-(3-methoxyphenyl)cyclopentane-1,2-diol (17.2 mg, 26%) as a white solid. LCMS (conditions ZORBAX SB-Aq, 50*4.6 mm, 1.8 μm, Mobile Phase A: Water/0.05% TFA, Mobile Phase B: Acetonitrile/0.05% TFA, 55%-95% B-3 min): T_{R}=1.78 min; [M+H]⁺: 455, 457. ¹H NMR (400 MHz, DMSO-d₆) δ 9.32 (s, 1H), 7.94 (s, 1H), 7.75 (d, J=2.2 Hz, 1H), 7.73-7.65 (m, 1H), 7.31-7.20 (m, 1H), 7.01 (d, J=7.8 Hz, 1H), 7.71 (s, 1H), 6.81 (dd, J=7.8, 2.6 Hz, 1H), 6.66 (d, J=2.2 Hz, 1H), 4.99 (d, J=6.2 Hz, 1H), 4.92 (d, J=6.2 Hz, 1H), 4.89-4.82 (m, 1H), 4.34-4.21 (m, 1H), 4.13-3.97 (m, 1H), 3.89 (s, 3H), 3.77 (s, 3H), 3.16-2.99 (m, 1H), 2.41-2.25 (m, 1H), 2.08-1.91 (m, 1H).

### Synthesis of (1R,2S,3R,5R)-3-(4-amino-2-chloro-7H-pyrrolo[2,3-d]pyrimidin-7-yl)-5-(3-(azetidin-1-ylmethyl)phenyl)cyclopentane-1,2-diol (672)

### 7-((3aS,4R,6R,6aR)-6-(3-(azetidin-1-ylmethyl)phenyl)-2,2-dimethyltetrahydro-4H-cyclopenta[d][1,3]dioxol-4-yl)-2-chloro-N-(4-methoxybenzyl)-7H-pyrrolo[2,3-d]pyrimidin-4-amine (303d)

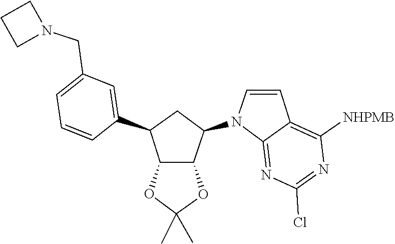

Into a 8 mL vial were added 3-[(3aR,4R,6R,6aS)-6-(2-chloro-4-{[(4-methoxyphenyl)methyl]amino}pyrrolo[2,3-d]pyrimidin-7-yl)-2,2-dimethyl-tetrahydro-3aH-cyclopenta[d][1,3]dioxol-4-yl]benzaldehyde (120.0 mg, 0.2 mmol, 1.0 equiv), azetidine (38.5 mg, 0.6 mmol, 3.0 equiv) and NaBH (OAc) 3 (143.1 mg, 0.6 mmol, 3.0 equiv), AcOH (27.0 mg, 0.4 mmol, 2.0 equiv) in DCM (2.0 mL) at room temperature. The resulting mixture was stirred for 0.5h at room temperature. The resulting mixture was concentrated under reduced pressure. The resulting mixture was diluted with DCM (3.0 mL). The residue was purified by silica gel column chromatography (1:1 Petroleum ether/THF) to afford 7-[(3aS,4R,6R,6aR)-6-[3-(azetidin-1-ylmethyl)phenyl]-2,2-dimethyl-tetrahydro-3aH-cyclopenta[d][1,3]dioxol-4-yl]-2-chloro-N-[(4-methoxyphenyl)methyl]pyrrolo[2,3-d]pyrimidin-4-amine 303d (120.0 mg, 92%) as a white solid. LCMS (conditions L-column3 ODS, 50*3.0 mm, 3.0 μm, Mobile Phase A: Water (0.05% ammonia water), Mobile Phase B: Acetonitrile, 5%-100% B-2 min, 1.20 L/min, ES, m/z): T_{R}=1.48 min; [M+H]⁺: 574, 576.

### (1R,2S,3R,5R)-3-(4-amino-2-chloro-7H-pyrrolo[2,3-d]pyrimidin-7-yl)-5-(3-(azetidin-1-ylmethyl)phenyl)cyclopentane-1,2-diol (677)

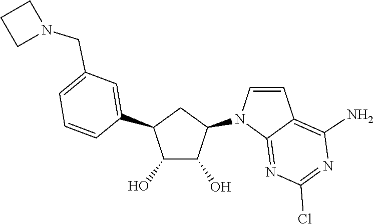 [SMILES: Nc1nc(Cl)nc2c1ccn2[C@@H]1C[C@H](c2cccc(CN3CCC3)c2)[C@@H](O)[C@H]1O]

Into a 8 mL vial were added 7-[(3aS,4R,6R,6aR)-6-[3-(azetidin-1-ylmethyl)phenyl]-2,2-dimethyl-tetrahydro-3aH-cyclopenta[d][1,3]dioxol-4-yl]-2-chloro-N-[(4-methoxyphenyl)methyl]pyrrolo[2,3-d]pyrimidin-4-amine 303d (60.0 mq, 0.1 mmol, 1.0 equiv) and TFA (2.0 mL) at room temperature. The resulting mixture was stirred for 0.5h at 60° C. After completion of reaction, the resulting mixture was concentrated under reduced pressure and was basified to pH >8 with NH₃·H₂O and was purified by Prep-HPLC with the following conditions (Column: Column: YMC-Actus Triart C18, 30*150 mm, 5 μm; Mobile Phase A: Water (10 mmol/L NH₄HCO₃+0.1% NH₃·H₂O), Mobile Phase B: ACN; Flow rate: 30 ml/min; Gradient: 20% B to 55% B in 7 min) to afford to afford (1R,2S,3R,5R)-3-{4-amino-2-chloropyrrolo[2,3-d]pyrimidin-7-yl}-5-[3-(azetidin-1-ylmethyl)phenyl]cyclopentane-1,2-diol 672 (12.7 mq, 29%) as an off-white solid. LCMS (conditions Xselect CSH Fluoro-Phenyl, 100*3.0 mm, 2.5 μm Mobile Phase A: Water/0.05% TFA, Mobile Phase B: Acetonitrile/0.05% TFA, 5%-40%-95% B-7 min, 1.00 mL/min, ES, m/z): T_{R}=2.228 min; [M+H]⁺: 414, 416. ¹H NMR (400 MHZ, DMSO-d₆) δ 7.45 (s, 2H), 7.40 (d, J=3.5 Hz, 1H), 7.31-7.21 (m, 3H), 7.15-7.08 (m, 1H), 6.60 (d, J=3.6 Hz, 1H), 4.93 (d, J=6.1 Hz, 1H), 4.88 (d, J=6.1 Hz, 1H), 4.87-4.76 (m, 1H), 4.26 (q, J=6.5 Hz, 1H), 4.03 (q, J=6.4 Hz, 1H), 3.52 (s, 2H), 3.13 (t, J=6.9 Hz, 4H), 3.09-3.01 (m, 1H), 2.40-2.29 (m, 1H), 2.04-1.89 (m, 3H).

### Synthesis of (1R,2S,3R,5R)-3-(4-amino-5-bromo-2-chloro-7H-pyrrolo[2,3-d]pyrimidin-7-yl)-5-(3-(azetidin-1-ylmethyl)phenyl)cyclopentane-1,2-diol (673)

### 5-bromo-7-((3aS,4R,6R,6aR)-6-(3-(((tert-butyldimethylsilyl)oxy)methyl)phenyl)-2,2-dimethyltetrahydro-4H-cyclopenta[d][1,3]dioxol-4-yl)-2,4-dichloro-7H-pyrrolo[2,3-d]pyrimidine (304d)

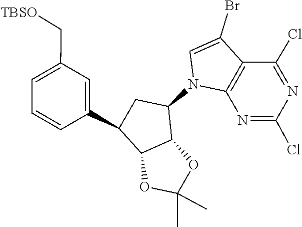 [SMILES: CC1(C)O[C@@H]2[C@H](O1)[C@@H](c1cccc(CO[Si](C)(C)C(C)(C)C)c1)C[C@H]2n1cc(Br)c2c(Cl)nc(Cl)nc21]

A solution of 5-bromo-2,4-dichloro-7H-pyrrolo[2,3-d]pyrimidine (1.7 g, 6.51 mmol, 1.0 equiv), (3aS,4S,6R,6aR)-6-(3-{[(tert-butyldimethylsilyl)oxy]methyl}phenyl)-2,2-dimethyl-tetrahydro-3aH-cyclopenta[d][1,3]dioxol-4-ol (3.2 q, 8.47 mmol, 1.3 equiv) and PPh₃ (3.4 g, 13.02 mmol, 2.0 equiv) in toluene (20.0 mL) was stirred for 5 min at room temperature under nitrogen atmosphere. To the above mixture was added DBAD (3.0 g, 13.02 mmol, 2.0 equiv) in toluene (20.0 mL) dropwise over 5 min at 0° C. The resulting mixture was stirred for 3 h at 50° C. The resulting mixture was concentrated under reduced pressure. The residue was purified by silica gel column chromatography (4:1 Petroleum ether/THF) to afford 7-[(3aS,4R,6R,6aR)-6-(3-{[(tert-butyldimethylsilyl)oxy]methyl}phenyl)-2,2-dimethyl-tetrahydro-3aH-cyclopenta[d][1,3]dioxol-4-yl]-5-bromo-2,4-dichloropyrrolo[2,3-d]pyrimidine 304d (3.0 g, 57%) as a white solid. LCMS (conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min, 1.20 L/min, ES, m/z,): T_{R}=2.90 min; [M+H]⁺: 626, 628.

### 5-bromo-7-((3aS,4R,6R,6aR)-6-(3-(((tert-butyldimethylsilyl)oxy)methyl)phenyl)-2,2-dimethyltetrahydro-4H-cyclopenta[d][1,3]dioxol-4-yl)-2-chloro-7H-pyrrolo[2,3-d]pyrimidin-4-amine (305d)

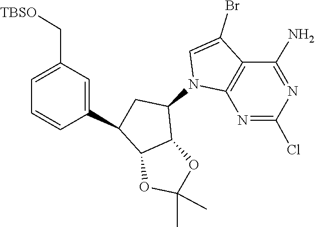 [SMILES: CC1(C)O[C@@H]2[C@H](O1)[C@@H](c1cccc(CO[Si](C)(C)C(C)(C)C)c1)C[C@H]2n1cc(Br)c2c(N)nc(Cl)nc21]

Into a 100 mL vial were added 7-[(3aS,4R,6R,6aR)-6-(3-{[(tert-butyldimethylsilyl)oxy]methyl}phenyl)-2,2-dimethyl-tetrahydro-3aH-cyclopenta[d][1,3]dioxol-4-yl]-5-bromo-2,4-dichloropyrrolo[2,3-d]pyrimidine 304d (3.0 q, 4.8 mmol, 1.0 equiv) and 7M NH₃ in MeOH (30.0 mL) at room temperature. The resulting mixture was stirred for 2 h at 80° C. The resulting mixture was concentrated under reduced pressure. The residue was purified by silica gel column chromatography (1:1 Petroleum ether/THF) to afford 7-[(3aS,4R,6R,6aR)-6-(3-{[(tert-butyldimethylsilyl)oxy]methyl}phenyl)-2,2-dimethyl-tetrahydro-3aH-cyclopenta[d][1,3]dioxol-4-yl]-5-bromo-2-chloropyrrolo[2,3-d]pyrimidin-4-amine 305d (2.7 g, 92%) as a white solid. LCMS (conditions L-column3 ODS, 50*3.0 mm, 3.0 μm, Mobile Phase A: Water (0.05% ammonia water), Mobile Phase B: Acetonitrile, 10%-95% B-3 min, 1.00 L/min, ES, m/z,): T_{R}=2.77 min; [M+H]⁺: 607, 609.

### 3-((3aR,4R,6R,6aS)-6-(4-amino-5-bromo-2-chloro-7H-pyrrolo[2,3-d]pyrimidin-7-yl)-2,2-dimethyltetrahydro-4H-cyclopenta[d][1,3]dioxol-4-yl)phenyl)methanol (306d)

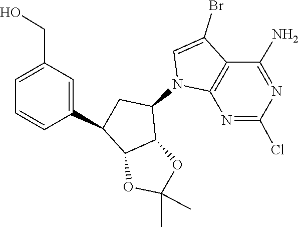 [SMILES: CC1(C)O[C@@H]2[C@H](O1)[C@@H](c1cccc(CO)c1)C[C@H]2n1cc(Br)c2c(N)nc(Cl)nc21]

Into a 100 mL vial were added 7-[(3aS,4R,6R,6aR)-6-(3-{[(tert-butyldimethylsilyl)oxy]methyl}phenyl)-2,2-dimethyl-tetrahydro-3aH-cyclopenta[d][1,3]dioxol-4-yl]-5-bromo-2-chloropyrrolo[2,3-d]pyrimidin-4-amine 305d (2.6 q, 4.3 mmol, 1.0 equiv) and THF (25.0 mL) at room temperature. To the above mixture was added 1M TBAF in THF (8.6 mL, 8.6 mmol, 2.0 equiv) dropwise at room temperature. The resulting mixture was stirred for 0.5h at room temperature. The resulting mixture was concentrated under reduced pressure. The residue was purified by silica gel column chromatography (1:1 Petroleum ether/THF) to afford {3-[(3aR,4R,6R,6aS)-6-{4-amino-5-bromo-2-chloropyrrolo[2,3-d]pyrimidin-7-yl}-2,2-dimethyl-tetrahydro-3aH-cyclopenta[d][1,3]dioxol-4-yl]phenyl}methanol 306d (1.6 g, 76%) as a white solid. LCMS (conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min, 1.20 L/min, ES, m/z,): T_{R}=1.84 min; [M+H]⁺: 493, 495.

### 3-((3aR,4R,6R,6aS)-6-(4-amino-5-bromo-2-chloro-7H-pyrrolo[2,3-d]pyrimidin-7-yl)-2,2-dimethyltetrahydro-4H-cyclopenta[d][1,3]dioxol-4-yl)benzaldehyde (307d)

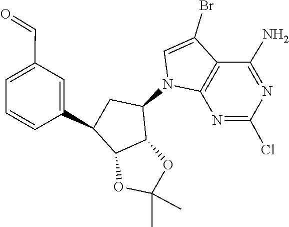 [SMILES: CC1(C)O[C@@H]2[C@H](O1)[C@@H](c1cccc(C=O)c1)C[C@H]2n1cc(Br)c2c(N)nc(Cl)nc21]

Into a 40 mL vial were added {3-[(3aR,4R,6R,6aS)-6-{4-amino-5-bromo-2-chloropyrrolo[2,3-d]pyrimidin-7-yl}-2,2-dimethyl-tetrahydro-3aH-cyclopenta[d][1,3]dioxol-4-yl]phenyl}methanol 306d (1.6 g, 3.3 mmol, 1.0 equiv) and manganese dioxide (1.4 g, 16.3 mmol, 5.0 equiv) in DCM (15.0 mL) at room temperature. The resulting mixture was stirred for overnight at room temperature. The resulting mixture was filtered, the filter cake was washed with DCM (3×20.0 mL) and concentrated under reduced pressure to afford 3-[(3aR,4R,6R,6aS)-6-{4-amino-5-bromo-2-chloropyrrolo[2,3-d]pyrimidin-7-yl}-2,2-dimethyl-tetrahydro-3aH-cyclopenta[d][1,3]dioxol-4-yl]benzaldehyde 307d (1.4 g, 88%) as a white solid. LCMS (conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min, 1.20 L/min, ES, m/z,): T_{R}=2.02 min; [M+H]⁺: 491, 493.

### (1R,2S,3R,5R)-3-(4-amino-5-bromo-2-chloro-7H-pyrrolo[2,3-d]pyrimidin-7-yl)-5-(3-(azetidin-1-ylmethyl)phenyl)cyclopentane-1,2-diol (673)

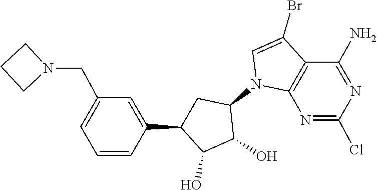 [SMILES: Nc1nc(Cl)nc2c1c(Br)cn2[C@@H]1C[C@H](c2cccc(CN3CCC3)c2)[C@@H](O)[C@H]1O]

Into a 20 mL vial were added 3-[(3aR,4R,6R,6aS)-6-{4-amino-5-bromo-2-chloropyrrolo[2,3-d]pyrimidin-7-yl}-2,2-dimethyl-tetrahydro-3aH-cyclopenta[d][1,3]dioxol-4-yl]benzaldehyde 307d (200.0 mg, 0.4 mmol, 1.0 equiv), azetidine (69.6 mg, 1.2 mmol, 3.0 equiv) and AcOH (48.8 mg, 0.8 mmol, 2.0 equiv), NaBH (OAc) 3 (258.5 mg, 1.2 mmol, 3.0 equiv) in DCM (2.0 mL) at room temperature. The resulting mixture was stirred for 0.5h at room temperature. The resulting mixture was concentrated under reduced pressure. The crude product mixture was used in the next step directly without further purification. The residue was dissolved into TFA (1.5 mL) at room temperature. The resulting mixture was stirred for additional 0.5h at 50° C. After completion of reaction, The resulting mixture was concentrated under reduced pressure and was basified to pH>8 with NH₃·H₂O and was purified by Prep-HPLC with the following conditions (Column: Column: YMC-Actus Triart C18, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 20% B to 55% B in 7 min) to afford (1R,2S,3R,5R)-3-{4-amino-5-bromo-2-chloropyrrolo[2,3-d]pyrimidin-7-yl}-5-[3-(azetidin-1-ylmethyl)phenyl]cyclopentane-1,2-diol 673 (23.1 mg, 26%) as a white solid. LCMS (conditions Cortecs C18+, 50*3.0 mm, 2.7 μm, Mobile Phase A: Water/0.05% TFA, Mobile Phase B: Acetonitrile/0.05% TFA, 5%-95% B-3 min, 1.50 L/min, ES, m/z,): T_{R}=0.953 min; [M+H]⁺: 492, 494. ¹H NMR (400 MHZ, DMSO-d₆) δ 7.76 (s, 1H), 7.29-7.21 (m, 3H), 7.11 (d, J=6.2 Hz, 1H), 4.96 (d, J=6.2 Hz, 1H), 4.91-4.80 (m, 2H), 4.26 (q, J=6.8 Hz, 1H), 3.98 (q, J=6.3 Hz, 1H), 3.52 (s, 2H), 3.12 (t, J=6.9 Hz, 4H), 3.09-2.99 (m, 1H), 2.34-2.26 (m, 1H), 2.04-1.89 (m, 3H).

### Synthesis of (1R,2S,3R,5R)-3-(4-amino-2-chloro-5-(1-methyl-1H-pyrazol-3-yl)-7H-pyrrolo[2,3-d]pyrimidin-7-yl)-5-(3-(azetidin-1-ylmethyl)phenyl)cyclopentane-1,2-diol (674)

### 7-((3aS,4R,6R,6aR)-6-(3-(((tert-butyldimethylsilyl)oxy)methyl)phenyl)-2,2-dimethyltetrahydro-4H-cyclopenta[d][1,3]dioxol-4-yl)-2-chloro-5-(1-methyl-1H-pyrazol-3-yl)-7H-pyrrolo[2,3-d]pyrimidin-4-amine (308d)

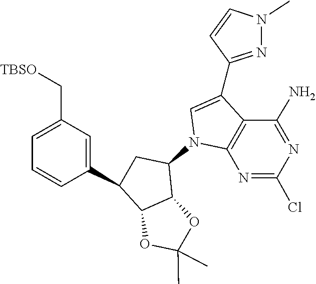 [SMILES: Cn1ccc(-c2cn([C@@H]3C[C@H](c4cccc(CO[Si](C)(C)C(C)(C)C)c4)[C@H]4OC(C)(C)O[C@H]43)c3nc(Cl)nc(N)c23)n1]

A solution of 7-[(3aS,4R,6R,6aR)-6-(3-{[(tert-butyldimethylsilyl)oxy]methyl}phenyl)-2,2-dimethyl-tetrahydro-3aH-cyclopenta[d][1,3]dioxol-4-yl]-5-bromo-2-chloropyrrolo[2,3-d]pyrimidin-4-amine 305d (190.0 mg, 0.3 mmol, 1.0 equiv),1-methyl-3-(4,4,5,5-tetramethyl-1,3,2-dioxaborolan-2-yl)-1H-pyrazole (130.0 mg, 0.6 mmol, 2.0 equiv) and K₃PO₄ (262.8 mg, 1.2 mmol, 3.0 equiv), Pd (DtBPF) Cl₂ (20.3 mg, 0.03 mmol, 0.1 equiv) in 1,4-dioxane (2.0 mL)/H₂O (0.2 mL) was stirred for 5 min at room temperature under nitrogen atmosphere. The resulting mixture was stirred for additional 0.5h at 80° C. The resulting mixture was concentrated under reduced pressure. The residue was purified by silica gel column chromatography (1:1 Petroleum ether/THF) to afford 7-[(3aS,4R,6R,6aR)-6-(3-{[(tert-butyldimethylsilyl)oxy]methyl}phenyl)-2,2-dimethyl-tetrahydro-3aH-cyclopenta[d][1,3]dioxol-4-yl]-2-chloro-5-(1-methylpyrazol-3-yl)pyrrolo[2,3-d]pyrimidin-4-amine 308d (110 mg, 57%) as a white solid. LCMS (conditions L-column3 ODS, 50*3.0 mm, 3.0 μm, Mobile Phase A: Water/0.05% Ammonia, Mobile Phase B: Acetonitrile, 5%-100% B-2 min, 1.20 L/min, ES, m/z,): T_{R}=1.60 min; [M+H]⁺: 609, 611.

### (3-((3aR,4R,6R,6aS)-6-(4-amino-2-chloro-5-(1-methyl-1H-pyrazol-3-yl)-7H-pyrrolo[2,3-d]pyrimidin-7-yl)-2,2-dimethyltetrahydro-4H-cyclopenta[d][1,3]dioxol-4-yl)phenyl)methanol (309d)

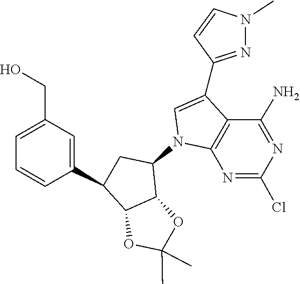 [SMILES: Cn1ccc(-c2cn([C@@H]3C[C@H](c4cccc(CO)c4)[C@H]4OC(C)(C)O[C@H]43)c3nc(Cl)nc(N)c23)n1]

Into a 8 mL vial were added 7-[(3aS,4R,6R,6aR)-6-(3-{[(tert-butyldimethylsilyl)oxy]methyl}phenyl)-2,2-dimethyl-tetrahydro-3aH-cyclopenta[d][1,3]dioxol-4-yl]-2-chloro-5-(1-methylpyrazol-3-yl)pyrrolo[2,3-d]pyrimidin-4-amine 308d (110.0 mg, 0.2 mmol, 1.0 equiv) and 1M TBAF in THF (0.36 mL, 0.4 mmol, 2.0 equiv) in THF (1.5 mL) at room temperature. The resulting mixture was stirred for 0.5h at room temperature. The resulting mixture was concentrated under reduced pressure. The residue was purified by silica gel column chromatography (1:1 Petroleum ether/THF) to afford {3-[(3aR,4R,6R,6aS)-6-[4-amino-2-chloro-5-(1-methylpyrazol-3-yl)pyrrolo[2,3-d]pyrimidin-7-yl]-2,2-dimethyl-tetrahydro-3aH-cyclopenta[d][1,3]dioxol-4-yl]phenyl}methanol 309d (75.0 mg, 83%) as a white solid. LCMS (conditions L-column3 ODS, 50*3.0 mm, 3.0 μm, Mobile Phase A: Water (0.05% ammonia water), Mobile Phase B: Acetonitrile, 10%-95% B-3 min, 1.00 L/min, ES, m/z,): T_{R}=2.12 min; [M+H]⁺: 495, 497.

### 3-((3aR,4R,6R,6aS)-6-(4-amino-2-chloro-5-(1-methyl-1H-pyrazol-3-yl)-7H-pyrrolo[2,3-d]pyrimidin-7-yl)-2,2-dimethyltetrahydro-4H-cyclopenta[d][1,3]dioxol-4-yl)benzaldehyde (310d)

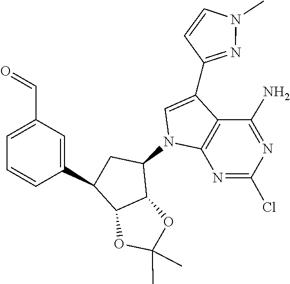 [SMILES: Cn1ccc(-c2cn([C@@H]3C[C@H](c4cccc(C=O)c4)[C@H]4OC(C)(C)O[C@H]43)c3nc(Cl)nc(N)c23)n1]

Into a 8 mL vial were added {3-[(3aR,4R,6R,6aS)-6-[4-amino-2-chloro-5-(1-methylpyrazol-3-yl)pyrrolo[2,3-d]pyrimidin-7-yl]-2,2-dimethyl-tetrahydro-3aH-cyclopenta[d][1,3]dioxol-4-yl]phenyl}methanol 309d (75.0 mq, 0.2 mmol, 1.0 equiv) and manganese dioxide (65.8 mq, 0.7 mmol, 5.0 equiv) in DCM (3.0 mL) at room temperature. The resulting mixture was stirred for overnight at room temperature. The resulting mixture was filtered, the filter cake was washed with DCM (5.0 mL). The filtrate was concentrated under reduced pressure to afford 3-[(3aR,4R,6R,6aS)-6-[4-amino-2-chloro-5-(1-methylpyrazol-3-yl)pyrrolo[2,3-d]pyrimidin-7-yl]-2,2-dimethyl-tetrahydro-3aH-cyclopenta[d][1,3]dioxol-4-yl]benzaldehyde 310d (70 mg, 97%) as a white solid. LCMS (conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min, 1.20 L/min, ES, m/z,): T_{R}=2.01 min; [M+H]⁺: 493, 495.

### 7-((3aS,4R,6R,6aR)-6-(3-(azetidin-1-ylmethyl)phenyl)-2,2-dimethyltetrahydro-4H-cyclopenta[d][1,3]dioxol-4-yl)-2-chloro-5-(1-methyl-1H-pyrazol-3-yl)-7H-pyrrolo[2,3-d]pyrimidin-4-amine (311d)

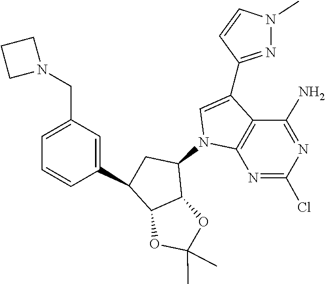 [SMILES: Cn1ccc(-c2cn([C@@H]3C[C@H](c4cccc(CN5CCC5)c4)[C@H]4OC(C)(C)O[C@H]43)c3nc(Cl)nc(N)c23)n1]

Into a 8 mL vial were added 3-[(3aR,4R,6R,6aS)-6-[4-amino-2-chloro-5-(1-methylpyrazol-3-yl)pyrrolo[2,3-d]pyrimidin-7-yl]-2,2-dimethyl-tetrahydro-3aH-cyclopenta[d][1,3]dioxol-4-yl]benzaldehyde 310d (75.0 mq, 0.1 mmol, 1.0 equiv), azetidine (26.0 mg, 0.4 mmol, 3.0 equiv), AcOH (6 mq, 0.1 mmol, 1.0 equiv) and NaBH (OAc) 3 (96.7 mq, 0.4 mmol, 3.0 equiv) in DCM (3.0 mL) at room temperature. The resulting mixture was stirred for 0.5h at room temperature. The resulting mixture was concentrated under reduced pressure. The residue was purified by silica gel column chromatography (1:1 Petroleum ether/THF) to afford 7-[(3aS,4R,6R,6aR)-6-[3-(azetidin-1-ylmethyl)phenyl]-2,2-dimethyl-tetrahydro-3aH-cyclopenta[d][1,3]dioxol-4-yl]-2-chloro-5-(1-methylpyrazol-3-yl)pyrrolo[2,3-d]pyrimidin-4-amine 311d (70 mg, 86%) as a white solid. LCMS (conditions L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-2 min, 1.20 L/min, ES, m/z,): T_{R}=1.51 min; [M+H]⁺: 534, 536.

### (1R,2S,3R,5R)-3-(4-amino-2-chloro-5-(1-methyl-1H-pyrazol-3-yl)-7H-pyrrolo[2,3-d]pyrimidin-7-yl)-5-(3-(azetidin-1-ylmethyl)phenyl)cyclopentane-12_diol/67/\

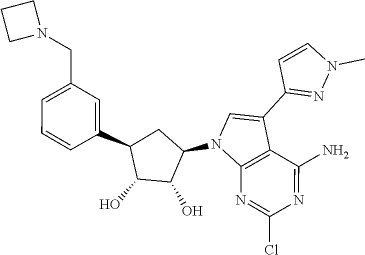 [SMILES: Cn1ccc(-c2cn([C@@H]3C[C@H](c4cccc(CN5CCC5)c4)[C@@H](O)[C@H]3O)c3nc(Cl)nc(N)c23)n1]

Into a 8 mL vial were added 7-[(3aS,4R,6R,6aR)-6-[3-(azetidin-1-ylmethyl)phenyl]-2,2-dimethyl-tetrahydro-3aH-cyclopenta[d][1,3]dioxol-4-yl]-2-chloro-5-(1-methylpyrazol-3-yl)pyrrolo[2,3-d]pyrimidin-4-amine 311d (70.0 mg, 0.1 mmol, 1.0 equiv) and TFA (1.0 mL) at room temperature. The resulting mixture was stirred for 0.5h at 50° C. After completion of reaction, the resulting mixture was concentrated under reduced pressure and was basified to pH>8 with NH₃·H₂O and was purified by Prep-HPLC with the following conditions (Column: Column: YMC-Actus Triart C18, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 20% B to 55% B in 7 min) to afford (1R,2S,3R,5R)-3-[4-amino-2-chloro-5-(1-methylpyrazol-3-yl)pyrrolo[2,3-d]pyrimidin-7-yl]-5-[3-(azetidin-1-ylmethyl)phenyl]cyclopentane-1,2-diol 674 (14.2 mq, 21%) as a white solid. LCMS (conditions Cortecs C18+, 50*3.0 mm, 2.7 μm, Mobile Phase A: Water/0.05% TFA, Mobile Phase B: Acetonitrile/0.05% TFA, 5%-95% B-3 min, 1.50 mL/min, ES, m/z): T_{R}=0.931 min; [M+H]⁺: 494, 496. 1H NMR (400 MHZ, DMSO-d₆) δ 9.32 (s, 1H), 7.94 (s, 1H), 7.75 (d, J=2.3 Hz, 1H), 7.72 (s, 1H), 7.34-7.23 (m, 3H), 7.13 (d, J=7.0 Hz, 1H), 6.66 (d, J=2.3 Hz, 1H), 5.00 (d, J=6.1 Hz, 1H), 4.93-4.82 (m, 2H), 4.30 (q, J=6.8 Hz, 1H), 4.04 (q, J=6.4 Hz, 1H), 3.89 (s, 3H), 3.57-3.53 (m, 2H), 3.24-3.04 (m, 5H), 2.41-2.30 (m, 1H), 2.06-1.93 (m, 3H).

### Synthesis of (1R,2S,3R,5R)-3-(4-amino-2-chloro-5-(4-chloro-1-methyl-1H-pyrazol-3-yl)-7H-pyrrolo[2,3-d]pyrimidin-7-yl)-5-(3-(azetidin-1-ylmethyl)phenyl)cyclopentane-1,2-diol (675)

### 7-((3aS,4R,6R,6aR)-6-(3-(((tert-butyldimethylsilyl)oxy)methyl)phenyl)-2,2-dimethyltetrahydro-4H-cyclopenta[d][1,3]dioxol-4-yl)-2-chloro-5-(4-chloro-1-methyl-1H-pyrazol-3-yl)-7H-pyrrolo[2,3-d]pyrimidin-4-amine (312d)

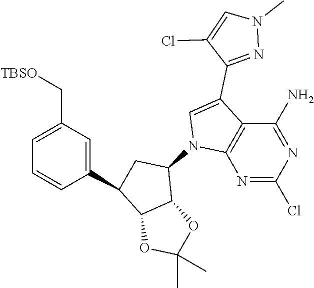 [SMILES: Cn1cc(Cl)c(-c2cn([C@@H]3C[C@H](c4cccc(CO[Si](C)(C)C(C)(C)C)c4)[C@H]4OC(C)(C)O[C@H]43)c3nc(Cl)nc(N)c23)n1]

Into a 40 mL vial were added mixture of 7-[(3aS,4R,6R,6aR)-6-(3-{[(tert-butyldimethylsilyl)oxy]methyl}phenyl)-2,2-dimethyl-tetrahydro-3aH-cyclopenta[d][1,3]dioxol-4-yl]-5-bromo-2-chloropyrrolo[2,3-d]pyrimidin-4-amine 305d (0.2 g, 0.3 mmol, 1.0 equiv) and 4-chloro-1-methyl-3-(4,4,5,5-tetramethyl-1,3,2-dioxaborolan-2-yl)pyrazole (0.16.g, 0.7 mmol, 2.0 equiv) in 1,4-dioxane (3.6 mL)/H₂O (0.4 mL) were added Pd (dtbpf) Cl₂ (21.4 mg, 0.03 mmol, 0.1 equiv) and K₃PO₄ (0.2. g, 0.99 mmol, 3.0 equiv) at room temperature under N₂ atmosphere. The resulting mixture was stirred for 2 h at 80° C. The resulting mixture was concentrated under reduced pressure. The residue was purified by silica gel column chromatography (1:1 Petroleum ether/AcOEt) to afford 7-[(3aS,4R,6R,6aR)-6-(3-{[(tert-butyldimethylsilyl)oxy]methyl}phenyl)-2,2-dimethyl-tetrahydro-3aH-cyclopenta[d][1,3]dioxol-4-yl]-2-chloro-5-(4-chloro-1-methylpyrazol-3-yl)pyrrolo[2,3-d]pyrimidin-4-amine 312d (0.18 g, 85%) as a white solid. LCMS (conditions XBridge C18, 50*3.0 mm, 3.5 μm, Mobile Phase A: Water/5 mM NH₃, Mobile Phase B: Acetonitrile, 5%-100% B-3 min, 1.50 L/min, ES, m/z,): T_{R}=2.53 min; [M+H]⁺: 643, 645.

### (3-((3aR,4R,6R,6aS)-6-(4-amino-2-chloro-5-(4-chloro-1-methyl-1H-pyrazol-3-yl)-7H-pyrrolo[2,3-d]pyrimidin-7-yl)-2,2-dimethyltetrahydro-4H-cyclopenta[d][1,3]dioxol-4-yl)phenyl)methanol (313d)

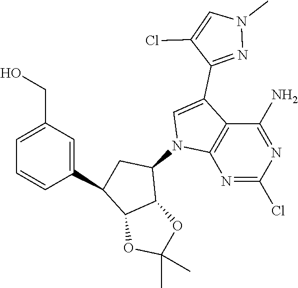 [SMILES: Cn1cc(Cl)c(-c2cn([C@@H]3C[C@H](c4cccc(CO)c4)[C@H]4OC(C)(C)O[C@H]43)c3nc(Cl)nc(N)c23)n1]

Into a 40 mL vial were added mixture of {3-[(3aR,4R,6R,6aS)-6-[4-amino-2-chloro-5-(4-chloro-1-methylpyrazol-3-yl)pyrrolo[2,3-d]pyrimidin-7-yl]-2,2-dimethyl-tetrahydro-3aH-cyclopenta[d][1,3]dioxol-4-yl]phenyl}methanol 312d (0.18 g, 0.34 mmol, 1.0 equiv) in THF (1.8 mL) was added 1M TBAF in THF (0.27 g, 1.0 mmol, 3.0 equiv) dropwise at room temperature. The resulting mixture was concentrated under reduced pressure. The residue was purified by silica gel column chromatography (1:10 THF/DCM) to afford7-[(3aS,4R,6R,6aR)-6-(3-{[(tert-butyldimethylsilyl)oxy]methyl}phenyl)-2,2-dimethyl-tetrahydro-3aH-cyclopenta[d][1,3]dioxol-4-yl]-2-chloro-5-(4-chloro-1-methylpyrazol-3-yl)pyrrolo[2,3-d]pyrimidin-4-amine 313d (0.12 g, 55%) as a white solid. LCMS (conditions XBridge C18, 50*3.0 mm, 3.5 μm, Mobile Phase A: Water/5 mM NH₄HCO₃, Mobile Phase B: Acetonitrile, 5%-100% B-2 min, 1.50 L/min, ES, m/z,): T_{R}=1.46 min; [M+H]⁺: 529, 531.

### 3-((3aR,4R,6R,6aS)-6-(4-amino-2-chloro-5-(4-chloro-1-methyl-1H-pyrazol-3-yl)-7H-pyrrolo[2,3-d]pyrimidin-7-yl)-2,2-dimethyltetrahydro-4H-cyclopenta[d][1,3]dioxol-4-yl)benzaldehyde (314d)

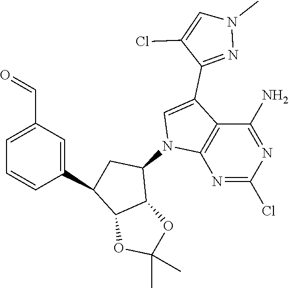 [SMILES: Cn1cc(Cl)c(-c2cn([C@@H]3C[C@H](c4cccc(C=O)c4)[C@H]4OC(C)(C)O[C@H]43)c3nc(Cl)nc(N)c23)n1]

Into a 40 mL vial were added mixture of {3-[(3aR,4R,6R,6aS)-6-[4-amino-2-chloro-5-(4-chloro-1-methylpyrazol-3-yl)pyrrolo[2,3-d]pyrimidin-7-yl]-2,2-dimethyl-tetrahydro-3aH-cyclopenta[d][1,3]dioxol-4-yl]phenyl}methanol 313d (0.12 g, 0.23 mmol, 1.0 equiv) and MnO2 (0.19 g, 2.3 mmol, 10 equiv) in DCM (5.0 mL) at room temperature. The resulting mixture was stirred for 14 h at room temperature. The resulting mixture was filtered, the filter cake was washed with DCM (3×5.0 mL). The filtrate was concentrated under reduced pressure to afford 3-[(3aR,4R,6R,6aS)-6-[4-amino-2-chloro-5-(4-chloro-1-methylpyrazol-3-yl)pyrrolo[2,3-d]pyrimidin-7-yl]-2,2-dimethyl-tetrahydro-3aH-cyclopenta[d][1,3]dioxol-4-yl]benzaldehyde 314d (0.1 mg, 84%) as a yellow solid. LCMS (conditions XBridge C18, 50*3.0 mm, 3.5 μm, Mobile Phase A: Water/5 mM NH₄HCO₃, Mobile Phase B: Acetonitrile, 5%-100% B-2 min, 1.50 L/min, ES, m/z,): T_{R}=1.57 min; [M+H]⁺: 527, 529.

### 7-((3aS,4R,6R,6aR)-6-(3-(azetidin-1-ylmethyl)phenyl)-2,2-dimethyltetrahydro-4H-cyclopenta[d][1,3]dioxol-4-yl)-2-chloro-5-(4-chloro-1-methyl-1H-pyrazol-3-yl)-7H-pyrrolo[2,3-d]pyrimidin-4-amine (315d)

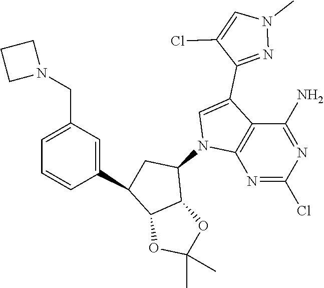 [SMILES: Cn1cc(Cl)c(-c2cn([C@@H]3C[C@H](c4cccc(CN5CCC5)c4)[C@H]4OC(C)(C)O[C@H]43)c3nc(Cl)nc(N)c23)n1]

Into a 40 mL vial were added mixture of 3-[(3aR,4R,6R,6aS)-6-[4-amino-2-chloro-5-(4-chloro-1-methylpyrazol-3-yl)pyrrolo[2,3-d]pyrimidin-7-yl]-2,2-dimethyl-tetrahydro-3aH-cyclopenta[d][1,3]dioxol-4-yl]benzaldehyde 314d (0.1 mg, 0.2 mmol, 1.0 equiv) and azetidine (32.5 mg, 0.6 mmol, 3.0 equiv) in DCM (2.0 mL) were added AcOH (22.8 mg, 0.4 mmol, 2.0 equiv) and NaBH (OAc) 3 (0.12 g, 0.6 mmol, 3.0 equiv) at room temperature. The resulting mixture was stirred for 0.5 h at room temperature. The resulting mixture was concentrated under reduced pressure. The residue was purified by silica gel column chromatography (1:8 MeOH/DCM) to afford 7-[(3aS,4R,6R,6aR)-6-[3-(azetidin-1-ylmethyl)phenyl]-2,2-dimethyl-tetrahydro-3aH-cyclopenta[d][1,3]dioxol-4-yl]-2-chloro-5-(4-chloro-1-methylpyrazol-3-yl)pyrrolo[2,3-d]pyrimidin-4-amine 315d (80 mg, 74%) as a white solid. LCMS (conditions XBridge C18, 50*3.0 mm, 3.5 μm, Mobile Phase A: Water/5 mM NH₄HCO₃, Mobile Phase B: Acetonitrile, 5%-100% B-2 min, 1.50 L/min, ES, m/z,): T_{R}=1.57 min; [M+H]⁺: 568, 570.

### (1R,2S,3R,5R)-3-(4-amino-2-chloro-5-(4-chloro-1-methyl-1H-pyrazol-3-yl)-7H-pyrrolo[2,3-d]pyrimidin-7-yl)-5-(3-(azetidin-1-ylmethyl)phenyl)cyclopentane-1,2-diol (675)

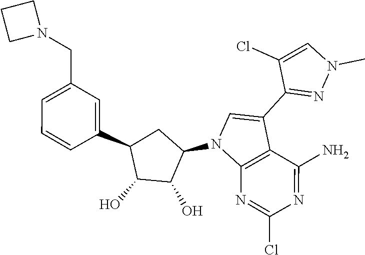 [SMILES: Cn1cc(Cl)c(-c2cn([C@@H]3C[C@H](c4cccc(CN5CCC5)c4)[C@@H](O)[C@H]3O)c3nc(Cl)nc(N)c23)n1]

Into a 40 mL vial were added mixture of 7-[(3aS,4R,6R,6aR)-6-[3-(azetidin-1-ylmethyl)phenyl]-2,2-dimethyl-tetrahydro-3aH-cyclopenta[d][1,3]dioxol-4-yl]-2-chloro-5-(4-chloro-1-methylpyrazol-3-yl)pyrrolo[2,3-d]pyrimidin-4-amine 315d (80 mg, 0.14 mmol, 1.0 equiv) in TFA (1 mL) at room temperature. The resulting mixture was concentrated under reduced pressure. The residue was dissolved into MeOH (3.0 mL) and was basified to pH>8 with NH₃·H₂O. The resulting mixture was purified by Prep-HPLC with the following conditions (Column: Bridge C18, 19*150 mm, 5 am; Mobile Phase A: 20 mM NH₄HCO₃+0.05% NH₃·H₂O, Mobile Phase B: ACN; Flow rate: 20 mL/min; Gradient: 10% to 65% in 8 min,) to afford (1R,2S,3R,5R)-3-[4-amino-2-chloro-5-(4-chloro-1-methylpyrazol-3-yl)pyrrole [2,3-d]pyrimidin-7-yl]-5-[3-(azetidin-1-ylmethyl)phenyl]cyclopentane-1,2-diol 675 (34.8 mg, 47%) as a white solid. LCMS (conditions Cortecs C18+, 50*3.0 mm, 2.7 μm, Mobile Phase A: Water/0.05% TFA, Mobile Phase B: Acetonitrile/0.05% TFA, 5%-95% B-3 min, 1.50 L/min, ES, m/z,): T_{R}=1.068 min; [M+H]⁺: 528, 530.1H NMR (400 MHZ, DMSO-d₆) δ 8.97 (s, 1H), 8.10 (s, 1H), 7.96 (s, 1H), 7.78 (s, 1H), 7.33-7.22 (m, 3H), 7.15-7.08 (m, 1H), 5.02 (d, J=6.2 Hz, 1H), 4.95-4.83 (m, 2H), 4.37 (q, J=6.6 Hz, 1H), 4.07 (q, J=6.2 Hz, 1H), 3.90 (s, 3H), 3.52 (s, 2H), 3.16-3.05 (m, 5H), 2.48-2.37 (m, 1H), 2.14-1.92 (m, 3H)

### Synthesis of (1R,2S,3R,5R)-3-{4-amino-2-chloropyrrolo[2,3-d]pyrimidin-7-yl}-5-[3-(difluoromethoxy)phenyl]cyclopentane-1,2-diol (676)

### (3aR,6R,6aR)-6-[3-(difluoromethoxy)phenyl]-2,2-dimethyl-tetrahydrocyclopenta[d][1,3]dioxol-4-one (252e)

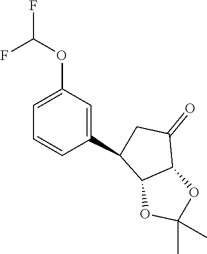 [SMILES: CC1(C)O[C@@H]2[C@@H](c3cccc(OC(F)F)c3)CC(=O)[C@@H]2O1]

A solution of (3aR,6aR)-2,2-dimethyl-3aH,6aH-cyclopenta[d][1,3]dioxol-4-one (0.4 g, 2.6 mmol, 1.0 eq), 2-[3-(difluoromethoxy)phenyl]-4,4,5,5-tetramethyl-1,3,2-dioxaborolane (0.9 g, 3.4 mmol, 1.3 eq) and Chloro(1,5-cyclooctadiene) rhodium (I) dimer (0.13 g, 0.26 mmol, 0.1 eq), KOH (168 μL, 1.13 eq) in MeOH (4.0 mL) at room temperature under nitrogen atmosphere. The final reaction mixture was irradiated with microwave radiation for 2 h at 40° C. The resulting mixture was concentrated under reduced pressure. The residue was dissolved in DCM (3.0 mL) and was purified by silica gel column chromatography (4:1 Petroleum ether: AcOEt) to afford (3aR,6R,6aR)-6-[3-(difluoromethoxy)phenyl]-2,2-dimethyl-tetrahydrocyclopenta[d][1,3]dioxol-4-one 252e (0.6 g, 78%) as a white solid. LCMS (conditions XBridge C18, 50*3.0 mm, 3.5 μm, Mobile Phase A: Water/5 mM NH₄HCO₃, Mobile Phase B: Methanol, 10%-95% B-3 min (+), 1.50 L/min, ES, m/z): T_{R}=2.05 min; [M+H]⁺: 299.

### (3aS,4S,6R,6aR)-6-[3-(difluoromethoxy)phenyl]-2,2-dimethyl-tetrahydro-3aH-cyclopenta[d][1,3]dioxol-4-ol (253e)

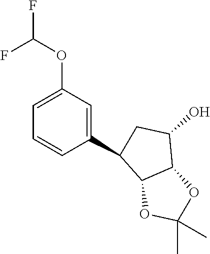 [SMILES: CC1(C)O[C@@H]2[C@H](O1)[C@@H](c1cccc(OC(F)F)c1)C[C@@H]2O]

Into a 40 mL vial were added (3aR,6R,6aR)-6-[3-(difluoromethoxy)phenyl]-2,2-dimethyl-tetrahydrocyclopenta[d][1,3]dioxol-4-one 252e (0.6 g, 2.0 mmol, 1.0 eq) and NaBH₄ (0.1 g, 3.0 mmol, 1.5 eq) in MeOH (6.0 mL) at 0° C. The resulting mixture was stirred for 0.5 h at room temperature. The resulting mixture was concentrated under reduced pressure. The residue was dissolved in DCM (2.0 mL) and was purified by silica gel column chromatography (2:1 Petroleum ether: AcOEt) to afford (3aS,4S,6R,6aR)-6-[3-(difluoromethoxy)phenyl]-2,2-dimethyl-tetrahydro-3aH-cyclopenta[d][1,3]dioxol-4-ol 253e (0.4 g, 66%) as a white solid. LCMS (conditions XBridge C18, 50*3.0 mm, 3.5 μm, Mobile Phase A: Water/5 mM NH₄HCO₃, Mobile Phase B: Methanol, 10%-95% B-2 min (+), 1.50 L/min, ES, m/z): T_{R}=1.38 min; [M+H]⁺: 301.

### 7-[(3aS,4R,6R,6aR)-6-[3-(difluoromethoxy)phenyl]-2,2-dimethyl-tetrahydro-3aH-cyclopenta[d][1,3]dioxol-4-yl]-2,4-dichloropyrrolo[2,3-d]pyrimidine (254e)

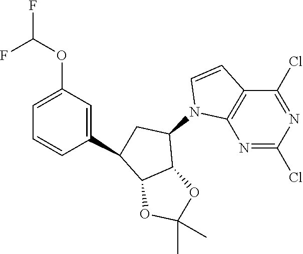 [SMILES: CC1(C)O[C@@H]2[C@H](O1)[C@@H](c1cccc(OC(F)F)c1)C[C@H]2n1ccc2c(Cl)nc(Cl)nc21]

A solution of 2,4-dichloro-7H-pyrrolo[2,3-d]pyrimidine (0.2 g, 1.0 mmol, 1.0 eq), (3aS,4S,6R,6aR)-6-[3-(difluoromethoxy)phenyl]-2,2-dimethyl-tetrahydro-3aH-cyclopenta[d][1,3]dioxol-4-ol 253e (0.4 g, 1.4 mmol, 1.3 eq) and DIAD (0.4 g, 2.1 mmol, 2.0 eq), PPh₃ (0.56 g, 2.1 mmol, 2.0 eq) in THF (3.0 mL) at 0° C. under nitrogen atmosphere. The resulting mixture was stirred for 12 h at room temperature. The resulting mixture was concentrated under reduced pressure. The residue was dissolved in DCM (2.0 mL) and was purified by silica gel column chromatography (2:1 Petroleum ether: AcOEt) to afford 7-[(3aS,4R,6R,6aR)-6-[3-(difluoromethoxy)phenyl]-2,2-dimethyl-tetrahydro-3aH-cyclopenta[d][1,3]dioxol-4-yl]-2,4-dichloropyrrolo[2,3-d]pyrimidine 254e (0.35 g, 70%) as a white solid. LCMS (conditions XBridge C18, 50*3.0 mm, 3.5 μm, Mobile Phase A: Water/5 mM NH₄HCO₃, Mobile Phase B: Acetonitrile, 5%-100% B-3 min (+), 1.50 L/min, ES, m/z,): T_{R}=2.31 min; [M+H]⁺: 470, 472.

### 7-[(3aS,4R,6R,6aR)-6-[3-(difluoromethoxy)phenyl]-2,2-dimethyl-tetrahydro-3aH-cyclopenta[d][1,3]dioxol-4-yl]-2-chloro-N-[(4-methoxyphenyl)methyl]pyrrolo[2,3-d]pyrimidin-4-amine (255e)

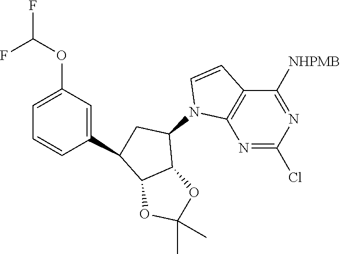

Into a 8 mL vial were added 7-[(3aS,4R,6R,6aR)-6-[3-(difluoromethoxy)phenyl]-2,2-dimethyl-tetrahydro-3aH-cyclopenta[d][1,3]dioxol-4-yl]-2,4-dichloropyrrolo[2,3-d]pyrimidine 254e (0.1 g, 0.2 mmol, 1.0 eq) and (4-methoxyphenyl)methanamine (87.5 mg, 0.6 mmol, 3.0 eq), TEA (43 mg, 0.4 mmol, 2.0 eq) in EtOH (1.0 mL) at room temperature. The resulting mixture was stirred for 2 h at 80° C. The resulting mixture was concentrated under reduced pressure. The residue was dissolved in DCM (1.0 mL) and was purified by silica gel column chromatography (1:1 Petroleum ether: AcOEt) to afford 7-[(3aS,4R,6R,6aR)-6-[3-(difluoromethoxy)phenyl]-2,2-dimethyl-tetrahydro-3aH-cyclopenta[d][1,3]dioxol-4-yl]-2-chloro-N-[(4-methoxyphenyl)methyl]pyrrolo[2,3-d]pyrimidin-4-amine 255e (70 mg, 58%) as a white solid. LCMS (conditions XBridge C18, 50*3.0 mm, 3.5 μm, Mobile Phase A: Water/5 mM NH₄HCO₃, Mobile Phase B: Acetonitrile, 5%-100% B-3 min (+), 1.50 L/min, ES, m/z,): T_{R}=2.30 min; [M+H]⁺: 571, 573.

### (1R,2S,3R,5R)-3-{4-amino-2-chloropyrrolo[2,3-d]pyrimidin-7-yl}-5-[3-(difluoromethoxy)phenyl]cyclopentane-1,2-diol (676)

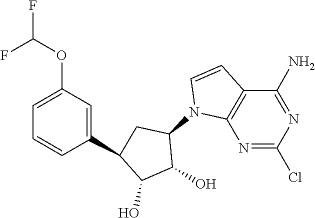 [SMILES: Nc1nc(Cl)nc2c1ccn2[C@@H]1C[C@H](c2cccc(OC(F)F)c2)[C@@H](O)[C@H]1O]

Into a 8 mL vial were added 7-[(3aS,4R,6R,6aR)-6-[3-(difluoromethoxy)phenyl]-2,2-dimethyl-tetrahydro-3aH-cyclopenta[d][1,3]dioxol-4-yl]-2-chloro-N-[(4-methoxyphenyl)methyl]pyrrolo[2,3-d]pyrimidin-4-amine 255e (70 mg, 0.1 mmol, 1.0 eq) and TFA (1.0 mL) at room temperature. The resulting mixture was stirred for 0.5 h at 80° C. The resulting mixture was concentrated under reduced pressure. The residue was dissolved in MeOH (3.0 mL). The mixture was basified to pH >8 with NH₃·H₂O and was purified by Prep-HPLC (column, Xbridge Prep C18 OBD column, 5 μm, 19*150 mm; mobile phase, Water (0.03% NH₄OH) and CH₃CN (20% CH₃CN up to 45% in 10 min); Detector, UV 220&254 nm) to afford (1R,2S,3R,5R)-3-{4-amino-2-chloropyrrolo[2,3-d]pyrimidin-7-yl}-5-[3-(difluoromethoxy)phenyl]cyclopentane-1,2-diol 676 (23.8 mg, 47%) as a white solid. LCMS (conditions: Xselect CSH Fluoro-Phenyl, 100*3.0 mm, 2.5 μm, Mobile Phase A: Water/0.05% TFA, Mobile Phase B: Acetonitrile/0.05% TFA, 20%-60%-95% B-7 min-1.50 mL/min, ES, m/z): T_{R}=2.89 min; [M+H]⁺: 411, 413. ¹H NMR (400 MHZ, DMSO-d₆+D₂O) δ 7.44-6.98 (m, 6H), 6.59 (d, J=3.6 Hz, 1H), 4.86-4.75 (m, 1H), 4.22 (t, J=6.6 Hz, 1H), 4.05 (t, J=6.7 Hz, 1H), 3.17-3.06 (m, 1H), 2.42-2.30 (m, 1H), 2.05-1.91 (m, 1H).

### Synthesis of (1R,2S,3R,5R)-3-{4-amino-5-bromo-2-chloropyrrolo[2,3-d]pyrimidin-7-yl}-5-(3,5-dimethoxyphenyl)cyclopentane-1,2-diol (677)

### 7-[(3aS,4R,6R,6aR)-6-(3,5-dimethoxyphenyl)-2,2-dimethyl-tetrahydro-3aH-cyclopenta[d][1,3]dioxol-4-yl]-5-bromo-2,4-dichloropyrrolo[2,3-d]pyrimidine (256e)

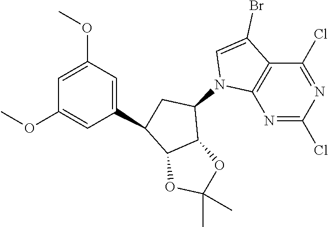 [SMILES: COc1cc(OC)cc([C@H]2C[C@@H](n3cc(Br)c4c(Cl)nc(Cl)nc43)[C@@H]3OC(C)(C)O[C@@H]32)c1]

A solution of 5-bromo-2,4-dichloro-7H-pyrrolo[2,3-d]pyrimidine (0.2 g, 0.75 mmol, 1.0 eq), (3aS,4S,6R,6aR)-6-(3-methoxyphenyl)-2,2-dimethyl-tetrahydro-3aH-cyclopenta[d][1,3]dioxol-4-ol 249e (0.26 g, 0.97 mmol, 1.3 eq) and DBAD (0.35 g, 1.5 mmol, 2.0 eq), PPh₃ (0.39 g, 1.5 mmol, 2.0 eq) in toluene (3.0 mL) at 0° C. under nitrogen atmosphere. The resulting mixture was stirred for 8 h at 60° C. The resulting mixture was concentrated under reduced pressure. The residue was dissolved in DCM (2.0 mL) and was purified by silica gel column chromatography (1:1 Petroleum ether: AcOEt) to afford 7-[(3aS,4R,6R,6aR)-6-(3,5-dimethoxyphenyl)-2,2-dimethyl-tetrahydro-3aH-cyclopenta[d][1,3]dioxol-4-yl]-5-bromo-2,4-dichloropyrrolo[2,3-d]pyrimidine 256e (0.3 g, 74%) as a white solid. LCMS (conditions XBridge C18, 50*3.0 mm, 3.5 μm, Mobile Phase A: Water/5 mM NH₄HCO₃, Mobile Phase B: Acetonitrile, 10%-95% B-3 min (+), 1.50 L/min, ES, m/z): T_{R}=2.41 min; [M+H]⁺: 542, 544.

### 7-[(3aS,4R,6R,6aR)-6-(3,5-dimethoxyphenyl)-2,2-dimethyl-tetrahydro-3aH-cyclopenta[d][1,3]dioxol-4-yl]-5-bromo-?-chloropyrrolo [2 3-d]pyrimidin-4-amine (257e)

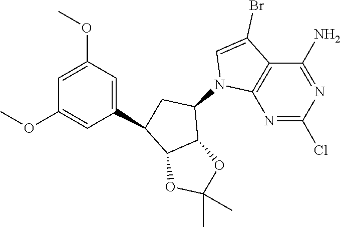 [SMILES: COc1cc(OC)cc([C@H]2C[C@@H](n3cc(Br)c4c(N)nc(Cl)nc43)[C@@H]3OC(C)(C)O[C@@H]32)c1]

Into a 8 mL vial were added 7-[(3aS,4R,6R,6aR)-6-(3,5-dimethoxyphenyl)-2,2-dimethyl-tetrahydro-3aH-cyclopenta[d][1,3]dioxol-4-yl]-5-bromo-2,4-dichloropyrrolo[2,3-d]pyrimidine 256e (0.2 g, 0.4 mmol, 1.0 eq) and 7M NH₃ in MeOH (2.0 mL) at room temperature. The resulting mixture was stirred for 8 h at 80° C. The resulting mixture was concentrated under reduced pressure. The residue was dissolved in DCM (2.0 mL) and was purified by silica gel column chromatography (1:2 Petroleum ether: AcOEt) to afford to afford 7-[(3aS,4R,6R,6aR)-6-(3,5-dimethoxyphenyl)-2,2-dimethyl-tetrahydro-3aH-cyclopenta[d][1,3]dioxol-4-yl]-5-bromo-2-chloropyrrolo[2,3-d]pyrimidin-4-amine 257e (0.12 g, 62%) as a yellow solid. LCMS (conditions XBridge C18, 50*3.0 mm, 3.5 μm, Mobile Phase A: Water/5 mM NH₄HCO₃, Mobile Phase B: Acetonitrile, 10%-95% B-3 min (+), 1.50 L/min, ES, m/z): T_{R}=2.13 min; [M+H]⁺: 523, 525.

### (1R,2S,3R,5R)-3-{4-amino-5-bromo-2-chloropyrrolo[2,3-d]pyrimidin-7-yl}-5-(3,5-dimethoxyphenyl)cyclopentane-1,2-diol (677)

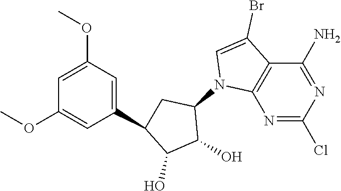 [SMILES: COc1cc(OC)cc([C@H]2C[C@@H](n3cc(Br)c4c(N)nc(Cl)nc43)[C@H](O)[C@@H]2O)c1]

Into a 8 mL vial were added 7-[(3aS,4R,6R,6aR)-6-(3,5-dimethoxyphenyl)-2,2-dimethyl-tetrahydro-3aH-cyclopenta[d][1,3]dioxol-4-yl]-5-bromo-2-chloropyrrolo[2,3-d]pyrimidin-4-amine 257e (50 mg, 0.1 mmol, 1.0 eq) and 4M HCl in MeOH (0.8 mL) at room temperature. The resulting mixture was stirred for 0.5 h at room temperature. The resulting mixture was concentrated under reduced pressure. The residue was dissolved in MeOH (3.0 mL) and basified to pH >8 with NH₃·H₂O. The residue was purified by Prep-HPLC (column, Xbridge Prep C18 OBD column, 5 μm, 19*150 mm; mobile phase, Water (0.03% NH₄OH) and CH₃CN (10% CH₃CN up to 50% in 10 min); Detector, UV 220&254 nm) to afford (1R,2S,3R,5R)-3-{4-amino-5-bromo-2-chloropyrrolo[2,3-d]pyrimidin-7-yl}-5-(3,5-dimethoxyphenyl)cyclopentane-1,2-diol 677 (27.1 mg, 29%) as an off-white solid. LCMS (conditions: Cortecs C18+, 50*3.0 mm, 2.7 μm, Mobile Phase A: Water/0.02% TFA, Mobile Phase B: Acetonitrile, 5%-95% B-3 min-1. 50 mL/min, ES, m/z): T_{R}=1.36 min; [M+H]⁺: 482, 484. ¹H NMR (400 MHZ, DMSO-d₆) δ 7.77 (s, 1H), 6.55 (d, J=2.3 Hz, 2H), 6.37 (t, J=2.3 Hz, 1H), 4.98-4.93 (m, 1H), 4.93-4.88 (m, 1H), 4.88-4.78 (m, 1H), 4.22 (q, J=6.7 Hz, 1H), 4.00 (q, J=6.3 Hz, 1H), 3.75 (s, 6H), 3.05-2.94 (m, 1H), 2.34-2.22 (m, 1H), 1.94 (q, J=11.9 Hz, 1H).

### Synthesis of (1R,2S,3R,5R)-3-[4-amino-2-chloro-5-(1-methylpyrazol-3-yl)pyrrolo[2,3-d]pyrimidin-7-yl]-5-(3,5-dimethoxyphenyl)cyclopentane-1,2-diol (678)

### 7-[(3aS,4R,6R,6aR)-6-(3,5-dimethoxyphenyl)-2,2-dimethyl-tetrahydro-3aH-cyclopenta[d][1,3]dioxol-4-yl]-2-chloro-5-(1-methylpyrazol-3-yl)pyrrolo[2,3-d]pyrimidin-4-amine (258e)

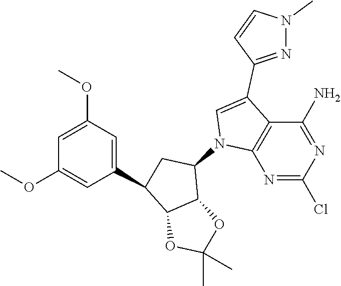 [SMILES: COc1cc(OC)cc([C@H]2C[C@@H](n3cc(-c4ccn(C)n4)c4c(N)nc(Cl)nc43)[C@@H]3OC(C)(C)O[C@@H]32)c1]

Into a 8 mL vial were added 7-[(3aS,4R,6R,6aR)-6-(3,5-dimethoxyphenyl)-2,2-dimethyl-tetrahydro-3aH-cyclopenta[d][1,3]dioxol-4-yl]-5-bromo-2-chloropyrrolo[2,3-d]pyrimidin-4-amine (90 mg, 0.17 mmol, 1.0 eq), 1-methyl-3-(4,4,5,5-tetramethyl-1,3,2-dioxaborolan-2-yl)pyrazole (71.5 mg, 0.34 mmol, 2.0 eq) and K₃PO₄ (0.1 g, 0.51 mmol, 3.0 eq), Pd (dtbpf) Cl₂ (14 mg, 0.02 mmol, 0.1 eq) in 1,4-dioxane (1.0 mL) and H₂O (0.1 mL) at room temperature. The resulting mixture was stirred for 2 h at 80° C. The resulting mixture was extracted with AcOEt (3×10 mL). The combined organic layers were washed with brine, dried over anhydrous Na₂SO₄. After filtration, the filtrate was concentrated under reduced pressure. The residue was purified by silica gel column chromatography (1:1 Petroleum ether: AcOEt) to afford 7-[(3aS,4R,6R,6aR)-6-(3,5-dimethoxyphenyl)-2,2-dimethyl-tetrahydro-3aH-cyclopenta[d][1,3]dioxol-4-yl]-2-chloro-5-(1-methylpyrazol-3-yl)pyrrolo[2,3-d]pyrimidin-4-amine 258e (70 mg, 78%) as a white solid. LCMS (conditions XBridge C18, 50*3.0 mm, 3.5 μm, Mobile Phase A: Water/5 mM NH₄HCO₃, Mobile Phase B: Acetonitrile, 10%-95% B-3 min (+), 1.50 L/min, ES, m/z): T_{R}=2.11 min; [M+H]⁺: 525, 527.

### (1R,2S,3R,5R)-3-[4-amino-2-chloro-5-(1-methylpyrazol-3-yl)pyrrolo[2,3-d]pyrimidin-7-yl]-5-(3,5-dimethoxyphenyl)cyclopentane-1,2-diol (678)

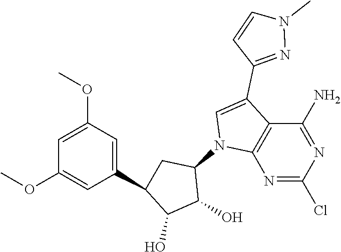 [SMILES: COc1cc(OC)cc([C@H]2C[C@@H](n3cc(-c4ccn(C)n4)c4c(N)nc(Cl)nc43)[C@H](O)[C@@H]2O)c1]

Into a 8 mL vial were added 3-[(3aR,4R,6R,6aS)-6-[4-amino-2-chloro-5-(1-methylpyrazol-3-yl)pyrrolo[2,3-d]pyrimidin-7-yl]-2,2-dimethyl-tetrahydro-3aH-cyclopenta[d][1,3]dioxol-4-yl]-5-methoxyphenol 258e (70 mg, 0.14 mmol, 1.0 eq) and 4M HCl in MeOH (1.0 mL) at room temperature. The resulting mixture was stirred for 0.5 h at room temperature. The resulting mixture was concentrated under reduced pressure. The residue was dissolved in MeOH (3.0 mL) and basified to pH >8 with NH₃·H₂O. The residue was purified by Prep-HPLC (column, Xbridge Prep C18 OBD column, 5 μm, 19*150 mm; mobile phase, Water (0.03% NH₄OH) and CH₃CN (15% CH₃CN up to 60% in 10 min); Detector, UV 220&254 nm) to afford (1R,2S,3R,5R)-3-[4-amino-2-chloro-5-(1-methylpyrazol-3-yl)pyrrolo[2,3-d]pyrimidin-7-yl]-5-(3,5-dimethoxyphenyl)cyclopentane-1,2-diol 678 (34.6 mg, 52%) as a white solid. LCMS (conditions: Cortecs C18+, 50*3.0 mm, 2.7 μm, Mobile Phase A: Water/0.02% TFA, Mobile Phase B: Acetonitrile, 5%-(25%-45%)-95% B-5 min-1.50 mL/min, ES, m/z): T_{R}=2.63 min; [M+H]⁺: 485, 487. 1H NMR (400 MHZ, DMSO-d₆) δ 9.31 (s, 1H), 7.94 (s, 1H), 7.75 (d, J=2.3 Hz, 1H), 7.71 (s, 1H), 6.65 (d, J=2.2 Hz, 1H), 6.56 (d, J=2.3 Hz, 2H), 6.38 (t, J=2.3 Hz, 1H), 4.98 (d, J=6.1 Hz, 1H), 4.92 (d, J=6.2 Hz, 1H), 4.90-4.79 (m, 1H), 4.27 (q, J=6.7 Hz, 1H), 4.07 (q, J=6.5 Hz, 1H), 3.89 (s, 3H), 3.76 (s, 6H), 3.10-2.99 (m, 1H), 2.39-2.27 (m, 1H), 1.97 (q, J=11.9 Hz, 1H).

### Synthesis of (1R,2S,3R,5R)-3-[4-amino-5-(1-benzylpyrazol-3-yl)-2-chloropyrrolo[2,3-d]pyrimidin-7-yl]-5-(3,5-dimethoxyphenyl)cyclopentane-1,2-diol (679)

### 7-[(3aS,4R,6R,6aR)-6-(3,5-dimethoxyphenyl)-2,2-dimethyl-tetrahydro-3aH-cyclopenta[d][1,3]dioxol-4-yl]-5-(1-benzylpyrazol-3-yl)-2-chloropyrrolo[2,3-d]pyrimidin-4-amine (259e)

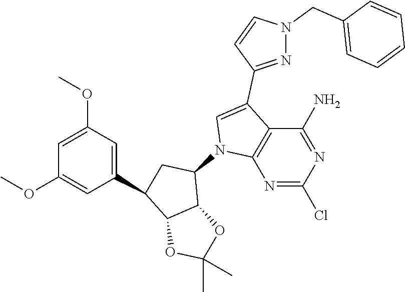 [SMILES: COc1cc(OC)cc([C@H]2C[C@@H](n3cc(-c4ccn(Cc5ccccc5)n4)c4c(N)nc(Cl)nc43)[C@@H]3OC(C)(C)O[C@@H]32)c1]

Into a 8 mL vial were added 7-[(3aS,4R,6R,6aR)-6-(3,5-dimethoxyphenyl)-2,2-dimethyl-tetrahydro-3aH-cyclopenta[d][1,3]dioxol-4-yl]-5-bromo-2-chloropyrrolo[2,3-d]pyrimidin-4-amine (0.1 g, 0.2 mmol, 1.0 eq), 1-benzylpyrazol-3-ylboronic acid (77.1 mg, 0.4 mmol, 2.0 eq) and K₃PO₄ (0.12 g, 0.573 mmol, 3.0 eq), Pd (dtbpf) Cl₂ (12.4 mg, 0.02 mmol, 0.1 eq) in 1,4-dioxane (1.0 ml) and H₂O (0.1 mL) at room temperature. The resulting mixture was stirred for additional 2 h at 80° C. The resulting mixture was concentrated under reduced pressure. The resulting mixture was extracted with AcOEt (3×10 mL). The combined organic layers were washed with brine, dried over anhydrous Na₂SO₄. After filtration, the filtrate was concentrated under reduced pressure. The residue was dissolved in DCM (3.0 mL) and was purified by silica gel column chromatography (1:1 Petroleum ether: AcOEt) to afford 7-[(3aS,4R,6R,6aR)-6-(3,5-dimethoxyphenyl)-2,2-dimethyl-tetrahydro-3aH-cyclopenta[d][1,3]dioxol-4-yl]-5-(1-benzylpyrazol-3-yl)-2-chloropyrrolo[2,3-d]pyrimidin-4-amine 259e (40 mg, 35%) as a white solid. LCMS (conditions XBridge C18, 50*3.0 mm, 3.5 μm, Mobile Phase A: Water/5 mM NH₄HCO₃, Mobile Phase B: Acetonitrile, 5%-100% B-3 min (+), 1.50 L/min, ES, m/z,): T_{R}=2.28 min; [M+H]⁺: 601, 603.

### (1R,2S,3R,5R)-3-[4-amino-5-(1-benzylpyrazol-3-yl)-2-chloropyrrolo[2,3-d]pyrimidin-7-yl]-5-(3,5-dimethoxyphenyl)cyclopentane-1,2-diol (679)

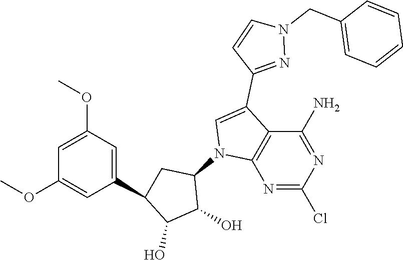 [SMILES: COc1cc(OC)cc([C@H]2C[C@@H](n3cc(-c4ccn(Cc5ccccc5)n4)c4c(N)nc(Cl)nc43)[C@H](O)[C@@H]2O)c1]

Into a 8 mL vial were added 7-[(3aS,4R,6R,6aR)-6-(3,5-dimethoxyphenyl)-2,2-dimethyl-tetrahydro-3aH-cyclopenta[d][1,3]dioxol-4-yl]-5-(1-benzylpyrazol-3-yl)-2-chloropyrrolo[2,3-d]pyrimidin-4-amine 259e (40 mg, 0.1 mmol, 1.0 eq) and 4M HCl in MeOH (0.5 mL) at room temperature. The resulting mixture was stirred for additional 0.5 h at room temperature. The resulting mixture was concentrated under reduced pressure. The residue was dissolved in MeOH (3.0 mL) and basified to pH >8 with NH₃·H₂O. The residue was purified by Prep-HPLC (column, Xbridge Prep C18 OBD column, 5 μm, 19*150 mm; mobile phase, Water (0.03% NH₄OH) and CH₃CN (25% CH₃CN up to 55% in 10 min); Detector, UV 220&254 nm) to afford (1R,2S,3R,5R)-3-[4-amino-5-(1-benzylpyrazol-3-yl)-2-chloropyrrolo[2,3-d]pyrimidin-7-yl]-5-(3,5-dimethoxyphenyl)cyclopentane-1,2-diol 679 (12.4 mg, 33%) as a white solid. LCMS (conditions: Xselect CSH Fluoro-Phenyl, 100*3.0 mm, 2.5 μm, Mobile Phase A: Water/0.05% TFA, Mobile Phase B: Acetonitrile/0.05% TFA, 20%-60%-95% B-7 min-1.50 mL/min, ES, m/z): T_{R}=4.32 min; [M+H]⁺: 561, 563. ¹H NMR (400 MHZ, DMSO-d₆+D₂O) δ 7.93-7.87 (m, 2H), 7.41-7.33 (m, 2H), 7.32-7.24 (m, 3H), 6.69 (d, J=2.3 Hz, 1H), 6.54 (d, J=2.3 Hz, 2H), 6.39-6.33 (m, 1H), 5.35 (s, 2H), 4.88-4.77 (m, 1H), 4.24 (t, J=6.9 Hz, 1H), 4.05 (t, J=6.7 Hz, 1H), 3.74 (s, 6H), 3.08-2.97 (m, 1H), 2.33-2.25 (m, 1H), 2.02-1.89 (m, 1H).

### 5-bromo-2,4-dichloro-7-((3aS,4R,6R,6aR)-6-(2-methoxypyridin-4-yl)-2,2-dimethyltetrahydro-4H-cyclopenta[d][1,3]dioxol-4-yl)-7H-pyrrolo[2,3-d]pyrimidine

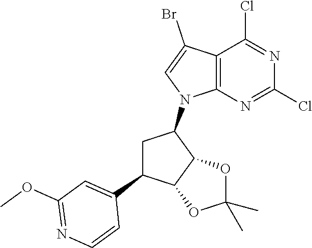 [SMILES: COc1cc([C@H]2C[C@@H](n3cc(Br)c4c(Cl)nc(Cl)nc43)[C@@H]3OC(C)(C)O[C@@H]32)ccn1]

To a solution of (3aS,4S,6R,6aR)-6-(2-methoxypyridin-4-yl)-2,2-dimethyltetrahydro-4H-cyclopenta[d][1,3]dioxol-4-ol (249 mg, 0.94 mmol, 1.5 eq.) in toluene (2 mL) was added 5-bromo-2,4-dichloro-7H-pyrrolo[2,3-d]pyrimidine (170 mg, 0.64 mmol, 1 eq.) and PPh₃ (333 mg, 1.27 mmol, 2 eq.) were added under N₂. The mixture was cooled to 0° C. and DBAD (286 g, 1.24 mmol, 1.9 eq) was added portionwise. The resulting mixture was stirred for additional 20 min at 0° C., then for 10 h at 50° C. After the reaction mixture was cooled down to RT, it was concentrated under reduced pressure and was purified by flash chromatography eluting with n-heptane/EtOAc (1-+20%) to give 5-bromo-2,4-dichloro-7-((3aS,4R,6R,6aR)-6-(2-methoxypyridin-4-yl)-2,2-dimethyltetrahydro-4H-cyclopenta[d][1,3]dioxol-4-yl)-7H-pyrrolo[2,3-d]pyrimidine (yield: 68 mg, 0.64 mmol, 20%), as a transparent syrup. R_{f}=0.20 (Cyclohexane/EtOAc 8:2). APCI: calcd. for C₂₀H₁₉BrCl₂N₄₀₃ [M+H]⁺: 513.0 found 514.8 ¹H NMR (400 MHZ, DMSO-d₆) δ 8.45 (s, 1H, H₆), 8.14 (d, J=5.4 Hz, 1H, a′-H, pyridine), 7.04 (dd, J=5.3, 1.5 Hz, 1H, B′-H, pyridine), 6.87-6.84 (m, 1H, B-H, pyridine), 5.28-5.20 (m, 1H, H₁′), 4.92 (dd, J=7.4, 5.0 Hz, 1H, H₂′), 4.76 (t, J=7.2 Hz, 1H, H₃′), 3.86 (s, 3H, OCH₃, pyridine), 3.38-3.27 (m, 1H, H₄′, under water peak), 2.61-2.52 (m, 2H, H₅′, under DMSO peak), 1.55 (s, 3H, CH₃, acetonide), 1.23 (s, 3H, CH₃, acetonide).

### 5-bromo-2-chloro-7-((3aS,4R,6R,6aR)-6-(2-methoxypyridin-4-yl)-2,2-dimethyltetrahydro-4H-cyclopenta[d][1,3]dioxol-4-yl)-7H-pyrrolo[2,3-d]pyrimidin-4-amine

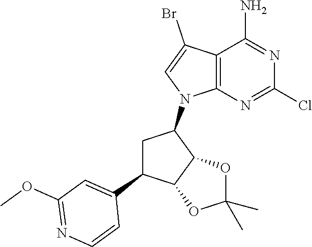 [SMILES: COc1cc([C@H]2C[C@@H](n3cc(Br)c4c(N)nc(Cl)nc43)[C@@H]3OC(C)(C)O[C@@H]32)ccn1]

To a solution of 5-bromo-2,4-dichloro-7-((3aS,4R,6R,6aR)-6-(2-methoxypyridin-4-yl)-2,2-dimethyltetrahydro-4H-cyclopenta[d][1,3]dioxol-4-yl)-7H-pyrrolo[2,3-d]pyrimidine (63 mg, 0.12 mmol, 1 eq.) in dioxane (3 mL) in a pressure flask was added aqueous ammonia (25%, 6 mL). The reaction mixture was stirred for 18 h at 100° C. After concentration, the residue was purified by flash chromatography eluting with DCM/MeOH (0.5->5%) to give 5-bromo-2-chloro-7-((3aS,4R,6R,6aR)-6-(2-methoxypyridin-4-yl)-2,2-dimethyltetrahydro-4H-cyclopenta[d][1,3]dioxol-4-yl)-7H-pyrrolo[2,3-d]pyrimidin-4-amine (yield: 27 mg, 0.05 mmol, 41%), as a brown solid. R_{f}=0.58 (DCM/MeOH 95:5). APCI: calcd. for C₂₀H₂₁BrClN₅O₃ [M+H]⁺: 494.1 found 494.2/496.2 ¹H NMR (400 MHZ, DMSO-d₆) δ 8.11 (d, J=5.4 Hz, 1H, a′-H, pyridine), 7.83 (s, 1H, H₆), 7.01 (dd, J=5.3, 1.5 Hz, 1H, B′-H, pyridine), 6.83-6.81 (m, 1H, B-H, pyridine), 5.11-5.03 (m, 1H, H₁′), 4.83 (dd, J=7.5, 5.0 Hz, 1H, H₂′), 4.71 (t, J=7.2 Hz, 1H, H₃′), 3.84 (s, 3H, OCH₃, pyridine), 3.29-3.21 (m, 1H, H₄′), 2.45-2.34 (m, 2H, H₅′), 1.52 (s, 3H, CH₃, acetonide), 1.21 (s, 3H, CH₃, acetonide).

### (1R,2S,3R,5R)-3-(4-amino-5-bromo-2-chloro-7H-pyrrolo[2,3-d]pyrimidin-7-yl)-5-(2-methoxypyridin-4-yl)cyclopentane-1,2-diol (680)

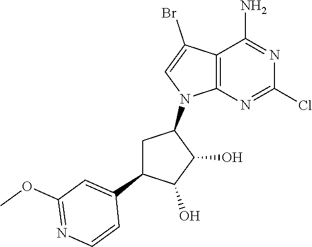 [SMILES: COc1cc([C@H]2C[C@@H](n3cc(Br)c4c(N)nc(Cl)nc43)[C@H](O)[C@@H]2O)ccn1]

5-bromo-2-chloro-7-((3aS,4R,6R,6aR)-6-(2-methoxypyridin-4-yl)-2,2-dimethyltetrahydro-4H-cyclopenta[d][1,3]dioxol-4-yl)-7H-pyrrolo[2,3-d]pyrimidin-4-amine (23 mg, 0.05 mmol, 1 eq.) was dissolved in 1.9 mL of TFA/water (4:1) and the mixture was stirred for 4 h at room temperature. After evaporation under reduced pressure, the residue was purified by preparative HPLC to give 680 (yield: 22 mg, 0.05 mmol, quant.), as a white foam. APCI: calcd. for C₁₇H₁₇BrClNO₃ [M+H]⁺: 454.0 found 454.0/456.0 HPLC (method B1): t_{R}=10.56 min, UV-purity at 210 nm=97%. ¹H NMR (400 MHZ, DMSO-d₆) δ 8.11 (d, J=5.3 Hz, 1H, a′-H, pyridine), 7.77 (s, 1H, H₆), 7.07 (dd, J=5.4, 1.5 Hz, 1H, B′-H, pyridine), 6.87-6.84 (m, 1H, B-H, pyridine), 4.90-4.83 (m, 1H, H₁′), 4.20 (t, J=6.8 Hz, 1H, H₂′), 4.01 (t, J=6.6 Hz, 1H, H₃′), 3.86 (s, 3H, OCH₃, pyridine), 3.11-3.03 (m, 1H, H₄′), 2.37-2.29 (m, 1H, H₅A′), 1.95 (q, J=11.6 Hz, 1H, H₅B′).

### Synthesis of (1R,2S,3R,5R)-3-(4-amino-2-chloro-7H-pyrrolo[2,3-d]pyrimidin-7-yl)-5-(3-amino-5-(isothiazol-4-yl)phenyl)cyclopentane-1,2-diol (681)

### N-(3-bromo-5-(4,4,5,5-tetramethyl-1,3,2-dioxaborolan-2-yl)phenyl) acetamide (128f)

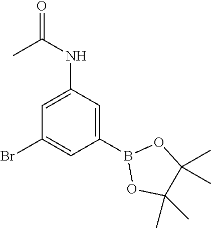 [SMILES: CC(=O)Nc1cc(Br)cc(B2OC(C)(C)C(C)(C)O2)c1]

A mixture of N-(3,5-dibromophenyl) acetamide (1.50 g, 5.12 mmol, 1.0 eq) [WO2016122150], bis(pinacolato)diboron (1.30 g, 5 mmol, 1.0 eq), KOAc (1.50 g, 15 mmol, 3.0 eq) and Pd (dppf) Cl₂ (208 mg, 0.26 mmol, 0.05 eq) in dioxane (15.0 mL) was stirred for 12 h at 80° C. under N₂ atmosphere. The resulting mixture was concentrated under reduced pressure and was extracted with ethyl acetate (3×50 mL). The combined organic layers were washed with brine and dried over anhydrous Na₂SO₄. After filtration, the filtrate was concentrated under reduced pressure. The residue was purified by silica gel column chromatography (7:3 petroleum ether: ethyl acetate) to afford N-[3-bromo-5-(4,4,5,5-tetramethyl-1,3,2-dioxaborolan-2-yl)phenyl]acetamide 128f (0.68 g, 39%) as a colourless oil. LCMS (conditions: L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min, T_{R}=1.77 min; ES m/z [M+H]⁺: 340, 342.

### N-(3-Bromo-5-((3aR,4R,6aR)-2,2-dimethyl-6-oxotetrahydro-4H-cyclopenta[d][1,3]dioxol-4-yl)phenyl) acetamide (129f)

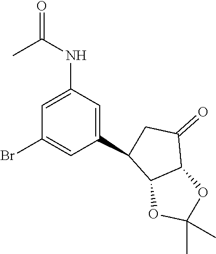 [SMILES: CC(=O)Nc1cc(Br)cc([C@H]2CC(=O)[C@@H]3OC(C)(C)O[C@H]23)c1]

Into a 10 mL microwave tube were added N-[3-bromo-5-(4,4,5,5-tetramethyl-1,3,2-dioxaborolan-2-yl)phenyl]acetamide 128f (0.68 g, 2.0 mmol, 1.0 eq), (3aR,6aR)-2,2-dimethyl-3aH,6aH-cyclopenta[d][1,3]dioxol-4-one (0.30 g, 1.9 mmol, 1.0 eq), 2M KOH in water (0.15 mL, 0.29 mmol, 0.15 eq), chloro(1,5-cyclooctadiene) rhodium (I) dimer (48.0 mg, 0.1 mmol, 0.05 eq) and CH₃OH (5.0 mL). The reaction mixture was irradiated with microwave radiation for 2 h at 40° C. The resulting mixture was concentrated under reduced pressure and water was added. The aqueous layer was extracted with ethyl acetate (3×50 mL). The combined organic phase was washed with brine, dried over anhydrous Na₂SO₄ and concentrated under reduced pressure to give crude product which was further purified by column chromatography (3:2 petroleum ether: ethyl acetate) to afford N-{3-[(3aR,4R,6aR)-2,2-dimethyl-6-oxo-tetrahydrocyclopenta[d][1,3]dioxol-4-yl]-5-bromophenyl}acetamide 129f (0.40 g, 55%) as a yellow solid. LCMS (conditions: Proshell HPH-C18, 50*3.0 mm, 2.7 μm, Mobile Phase A: Water (0.05% ammonia water), Mobile Phase B: Acetonitrile, 5%-95% B-3 min, T_{R}=1.13 min; ES m/z [M−H]: 366, 368.

### N-(3-((3aR,4R,6aR)-2,2-Dimethyl-6-oxotetrahydro-4H-cyclopenta[d][1,3]dioxol-4-yl)-5-(isothiazol-4-yl)phenyl) acetamide (130f)

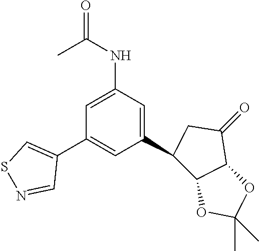 [SMILES: CC(=O)Nc1cc(-c2cnsc2)cc([C@H]2CC(=O)[C@@H]3OC(C)(C)O[C@H]23)c1]

A mixture of N-{3-[(3aR,4R,6aR)-2,2-dimethyl-6-oxo-tetrahydrocyclopenta[d][1,3]dioxol-4-yl]-5-bromophenyl}acetamide 129f (0.40 g, 1.1 mmol, 1.0 eq), 4-(4,4,5,5-tetramethyl-1,3,2-dioxaborolan-2-yl) isothiazole (0.35 g, 1.65 mmol, 1.5 eq) [WO2015100117], K₂CO₃ (0.45 g, 3.3 mmol, 3.0 eq) and Pd (dppf) Cl₂ (39.7 mg, 0.054 mmol, 0.05 eq) in 1,4-dioxane/H₂O=10/1 (5.5 mL) was stirred for 1 h at 80° C. under N₂ atmosphere. The resulting mixture was concentrated under reduced pressure and water was added. The resulting mixture was extracted with ethyl acetate (3×30 mL). The combined organic layers were washed with brine and dried over anhydrous Na₂SO₄. After filtration, the filtrate was concentrated under reduced pressure. The residue was purified by silica gel column chromatography (5:4 petroleum ether:ethyl acetate) to afford N-{3-[(3aR,4R,6aR)-2,2-dimethyl-6-oxo-tetrahydrocyclopenta[d][1,3]dioxol-4-yl]-5-(1,2-thiazol-4-yl)phenyl}acetamide 130f (0.35 g, 86%) as a yellow oil. LCMS (conditions: L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min, T_{R}=1.59 min; ES m/z [M+H]⁺: 373.

### N-(3-((3aR,4R,6S,6aS)-6-Hydroxy-2,2-dimethyltetrahydro-4H-cyclopenta[d][1,3]dioxol-4-yl)-5-(isothiazol-4-yl)phenyl) acetamide (131f)

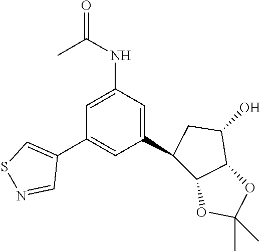 [SMILES: CC(=O)Nc1cc(-c2cnsc2)cc([C@H]2C[C@H](O)[C@@H]3OC(C)(C)O[C@@H]32)c1]

A mixture of N-{3-[(3aR,4R,6aR)-2,2-dimethyl-6-oxo-tetrahydrocyclopenta[d][1,3]dioxol-4-yl]-5-(1,2-thiazol-4-yl)phenyl}acetamide 130f (0.35 g, 0.94 mmol, 1.0 eq) and NaBH₄ (71 mg, 1.9 mmol, 2.0 eq) in MeOH (4.0 mL) was stirred for 0.5h at 0° C. After completion, the reaction mixture was quenched with ammonium chloride solution. The aqueous layer was extracted with ethyl acetate (3×30 mL). The combined organic phase was washed with brine, dried over anhydrous Na₂SO₄ and concentrated under reduced pressure. The residue was purified by silica gel column chromatography (1:1 petroleum ether:ethyl acetate) to afford N-{3-[(3aR,4R,6S,6aS)-6-hydroxy-2,2-dimethyl-tetrahydro-3aH-cyclopenta[d][1,3]dioxol-4-yl]-5-(1,2-thiazol-4-yl)phenyl}acetamide 131f (0.31 g, 88%) as a colourless oil. LCMS (conditions: Cortecs C18+, 50*3.0 mm, 2.7 μm, Mobile Phase A: Water/0.05% TFA, Mobile Phase B: Acetonitrile, 2%-100% B-3 min, T_{R}=1.26 min; ES m/z [M+H]⁺: 375.

### N-(3-((3aR,4R,6R,6aS)-6-(2,4-Dichloro-7H-pyrrolo[2,3-d]pyrimidin-7-yl)-2,2-dimethyltetrahydro-4H-cyclopenta[d][1,3]dioxol-4-yl)-5-(isothiazol-4-yl)phenyl) acetamide (132f)

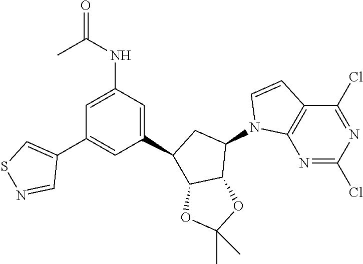 [SMILES: CC(=O)Nc1cc(-c2cnsc2)cc([C@H]2C[C@@H](n3ccc4c(Cl)nc(Cl)nc43)[C@@H]3OC(C)(C)O[C@@H]32)c1]

To a stirred mixture of N-{3-[(3aR,4R,6S,6aS)-6-hydroxy-2,2-dimethyl-tetrahydro-3aH-cyclopenta[d][1,3]dioxol-4-yl]-5-(1,2-thiazol-4-yl)phenyl}acetamide 131 (0.31 g, 0.83 mmol, 1.2 eq), PPh₃ (0.36 g, 1.38 mmol, 2.0 eq) and 2,4-dichloro-7H-pyrrolo[2,3-d]pyrimidine (0.13 g, 0.69 mmol, 1.0 eq) in THF (3.0 mL) was added DIAD (0.28 g, 1.38 mmol, 2.0 eq) in THF (1.0 mL) dropwise at 0° C. The resulting mixture was stirred for 8 h at room temperature, then concentrated under vacuum and quenched by addition of water. The aqueous layer was extracted with ethyl acetate (3×30 mL). The combined organic phase was washed with brine, dried over anhydrous Na₂SO₄ and concentrated under reduced pressure to give crude product which was further purified by column chromatography (2:3 tetrahydrofuran: petroleum ether) to afford N-{3-[(3aR,4R,6R,6aS)-6-{2,4-dichloropyrrolo[2,3-d]pyrimidin-7-yl}-2,2-dimethyl-tetrahydro-3aH-cyclopenta[d][1,3]dioxol-4-yl]-5-(1,2-thiazol-4-yl)phenyl}acetamide 132f (0.15 g, 40%) as a light yellow oil. LCMS (conditions: Cortecs C18+, 50*3.0 mm, 2.7 μm, Mobile Phase A: Water/0.05% TFA, Mobile Phase B: Acetonitrile, 2%-100% B-3 min, T_{R}=1.30 min; ES m/z [M+H]⁺: 544, 546.

### N-(3-((3aR,4R,6R,6aS)-6-(4-amino-2-chloro-7H-pyrrolo[2,3-d]pyrimidin-7-yl)-2,2-dimethyltetrahydro-4H-cyclopenta[d][1,3]dioxol-4-yl)-5-(isothiazol-4-yl)phenyl) acetamide (133f)

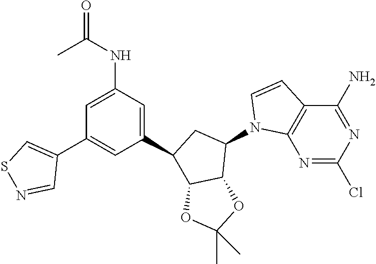 [SMILES: CC(=O)Nc1cc(-c2cnsc2)cc([C@H]2C[C@@H](n3ccc4c(N)nc(Cl)nc43)[C@@H]3OC(C)(C)O[C@@H]32)c1]

N-{3-[(3aR,4R,6R,6aS)-6-{2,4-Dichloropyrrolo[2,3-d]pyrimidin-7-yl}-2,2-dimethyl-tetrahydro-3aH-cyclopenta[d][1,3]dioxol-4-yl]-5-(1,2-thiazol-4-yl)phenyl}acetamide 132f (0.15 g, 0.28 mmol, 1.0 eq) was dissolved in 7M NH₃ in MeOH (2.0 mL). The resulting solution was stirred for 8 h at 80° C., then concentrated under reduced pressure to give crude product which was further purified by column chromatography (1:1 tetrahydrofuran: petroleum ether) to afford N-{3-[(3aR,4R,6R,6aS)-6-{4-amino-2-chloropyrrolo[2,3-d]pyrimidin-7-yl}-2,2-dimethyl-tetrahydro-3aH-cyclopenta[d][1,3]dioxol-4-yl]-5-(1,2-thiazol-4-yl)phenyl}acetamide 133f (80.0 mg, 55%) as a light yellow oil. LCMS (conditions: L-column3 ODS, 50*3.0 mm, 3.0 μm, Mobile Phase A: Water/5 mM NH₄HCO₃+0.05% Ammonia, Mobile Phase B: Acetonitrile/5% water, 5%-100% B-3 min, T_{R}=1.68 min; ES m/z [M+H]⁺: 525, 527.

### (1R,2S,3R,5R)-3-(4-Amino-2-chloro-7H-pyrrolo[2,3-d]pyrimidin-7-yl)-5-(3-amino-5-(isothiazol-4-yl)phenyl)cyclopentane-1,2-diol (681)

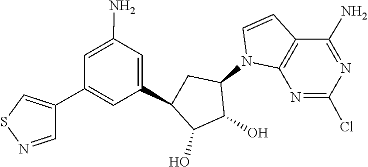 [SMILES: Nc1cc(-c2cnsc2)cc([C@H]2C[C@@H](n3ccc4c(N)nc(Cl)nc43)[C@H](O)[C@@H]2O)c1]

Into an 8 mL vial were added N-{3-[(3aR,4R,6R,6aS)-6-{4-amino-2-chloropyrrolo[2,3-d]pyrimidin-7-yl}-2,2-dimethyl-tetrahydro-3aH-cyclopenta[d][1,3]dioxol-4-yl]-5-(1,2-thiazol-4-yl)phenyl}acetamide 133f (40 mg, 0.076 mmol, 1.0 eq) and 4M HCl in MeOH (1.0 mL) at room temperature. The resulting mixture was stirred for 0.5h at 60° C., then concentrated under reduced pressure. The crude product was dissolved in MeOH (2.0 mL) and basified to pH>8 with NH₃·H₂O. The mixture was purified by Prep-HPLC with the following conditions (Column: Welch Ultimate XB-C18, 50*250 cm, 10 μm; Mobile Phase A: 0.1% HCl, Mobile Phase B: ACN; Flow rate: 90 mL/min; Gradient: 5% B-5% B-2 min, 5% B-35% B, 12 min.) to afford (1R,2S,3R,5R)-3-{4-amino-2-chloropyrrolo[2,3-d]pyrimidin-7-yl}-5-[3-amino-5-(1,2-thiazol-4-yl)phenyl]cyclopentane-1,2-diol HCl salt 681 (11.1 mg, 33%) as a white solid. LCMS (conditions: ZORBAX SB-Aq, 50*4.6 mm, 1.8 μm, Mobile Phase A: Water/0.05% ammonia water, Mobile Phase B: Acetonitrile, 5%-95% B-3 min, T_{R}=1.33 min; ES m/z [M+H]⁺: 443, 445. ¹H NMR (400 MHZ, DMSO-d₆+D₂O) δ 9.38 (s, 1H), 9.04 (s, 1H), 7.73 (s, 1H), 7.48 (s, 1H), 7.41 (d, J=3.6 Hz, 1H), 7.26 (s, 1H), 6.63 (d, J=3.5 Hz, 1H), 4.94-4.81 (m, 1H), 4.25 (t, J=6.5 Hz, 1H), 4.13 (t, J=7.0 Hz, 1H), 3.25-3.13 (m, 1H), 2.47-2.38 (m, 1H), 2.01 (q, J=11.8 Hz, 1H).

### Synthesis of (1R,2S,3R,5R)-3-{4-amino-2-chloropyrrolo[2,3-d]pyrimidin-7-yl}-5-(4-methoxyphenyl)cyclopentane-1,2-diol (682)

### (3aR,6R,6aR)-6-(4-Methoxyphenyl)-2,2-dimethyl-tetrahydrocyclopenta[d][1,3]dioxol-4-one (244g)

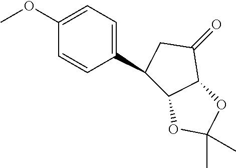

A solution of (3aR,6aR)-2,2-dimethyl-3aH,6aH-cyclopenta[d][1,3]dioxol-4-one (0.5 g, 3.2 mmol, 1.0 eq), 4-methoxyphenylboronic acid (0.64 g, 4.2 mmol, 1.3 eq), acetylacetonato bis(ethylene) rhodium (I)(84 mg, 0.3 mmol, 0.1 eq) and R-BINAP (0.26 g, 0.3 mmol, 0.1 eq) in 1,4-dioxane (5 mL) and H₂O (0.5 mL) was stirred for 3 h at 100° C. under nitrogen atmosphere. The reaction was quenched with water (10 mL) and then extracted with EtOAc (3×10 mL). The organic layer was dried over anhydrous Na₂SO₄, filtered and the filtrate was concentrated. The residue was dissolved in DCM (3 mL) and was purified by silica gel column chromatography (3:1 Petroleum ether: AcOEt) to afford (3aR,6R,6aR)-6-(4-methoxyphenyl)-2,2-dimethyl-tetrahydrocyclopenta[d][1,3]dioxol-4-one 244g (0.42 g, 49%) as a white solid. LCMS (conditions XBridge C18, 50*3.0 mm, 3.5 μm, Mobile Phase A: Water/5 mM NH₄HCO₃, Mobile Phase B: Acetonitrile, 10-95% B-2 min, 1.50 mL/min): T_{R}=1.39 min; ES m/z [M+H]⁺: 263.

### (3aS,45,6R,6aR)-6-(4-Methoxyphenyl)-2,2-dimethyl-tetrahydro-3aH-cyclopenta[d][1,3]dioxol-4-ol (245g)

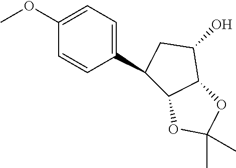

Into a 40 mL vial were added (3aR,6R,6aR)-6-(4-methoxyphenyl)-2,2-dimethyl-tetrahydrocyclopenta[d][1,3]dioxol-4-one 244g (0.42 g, 1.6 mmol, 1.0 eq) and NaBH₄ (91 mg, 2.4 mmol, 1.5 eq) in MeOH (4.0 mL) at 0° C. The resulting mixture was stirred for 0.5 h at room temperature. The reaction was quenched with water at 0° C. and then extracted with EtOAc (3×10 mL). The organic layer was dried over anhydrous Na₂SO₄, filtered and the filtrate was concentrated. The residue was dissolved in DCM (3 mL) and was purified by silica gel column chromatography (2:1 Petroleum ether: AcOEt) to afford (3aS,4S,6R,6aR)-6-(4-methoxyphenyl)-2,2-dimethyl-tetrahydro-3aH-cyclopenta[d][1,3]dioxol-4-ol 245g (0.26 g, 63%) as a white solid. LCMS (conditions XBridge C18, 50*3.0 mm, 3.5 μm, Mobile Phase A: Water/5 mM NH₄HCO₃, Mobile Phase B: Acetonitrile, 10-95% B-3 min, 1.50 mL/min): T_{R}=1.01 min; ES m/z [M+H]⁺: 265.

### 7-[(3aS,4R,6R,6aR)-6-(4-Methoxyphenyl)-2,2-dimethyl-tetrahydro-3aH-cyclopenta[d][1,3]dioxol-4-yl]-2,4-dichloropyrrolo[2,3-d]pyrimidine (246g)

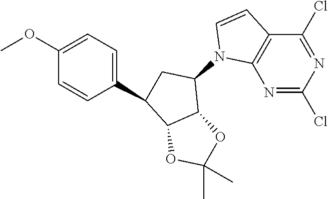 [SMILES: COc1ccc([C@H]2C[C@@H](n3ccc4c(Cl)nc(Cl)nc43)[C@@H]3OC(C)(C)O[C@@H]32)cc1]

A solution of (3aS,4S,6R,6aR)-6-(4-methoxyphenyl)-2,2-dimethyl-tetrahydro-3aH-cyclopenta[d][1,3]dioxol-4-ol 245g (0.26 g, 1.0 mmol, 1.5 eq), 2,4-dichloro-7H-pyrrolo[2,3-d]pyrimidine (0.12 g, 0.7 mmol, 1.0 eq), DIAD (0.27 g, 1.4 mmol, 2.0 eq) and PPh₃ (0.35 g, 1.4 mmol, 2.0 eq) in THF (3.0 mL) was stirred for 10 min at 0° C., then for 12 h at room temperature. The reaction was quenched with water (10 mL) and then extracted with EtOAc (3×10 mL). The organic layer was dried over anhydrous Na₂SO₄, filtered and the filtrate was concentrated. The residue was dissolved in DCM (3 mL) and was purified by silica gel column chromatography (2:1 Petroleum ether: AcOEt) to afford 7-[(3aS,4R,6R,6aR)-6-(4-methoxyphenyl)-2,2-dimethyl-tetrahydro-3aH-cyclopenta[d][1,3]dioxol-4-yl]-2,4-dichloropyrrolo[2,3-d]pyrimidine 246g (0.15 g, 50%) as a white solid. LCMS (conditions XBridge C18, 50*3.0 mm, 3.5 μm, Mobile Phase A: Water/5 mM NH₄HCO₃, Mobile Phase B: Acetonitrile, 10-95% B-3 min, 1.50 mL/min): T_{R}=2.33 min; ES m/z [M+H]⁺: 434, 436.

### 7-[(3aS,4R,6R,6aR)-6-(4-Methoxyphenyl)-2,2-dimethyl-tetrahydro-3aH-cyclopenta[d][1,3]dioxol-4-yl]-2-chloropyrrolo[2,3-d]pyrimidin-4-amine (247a)

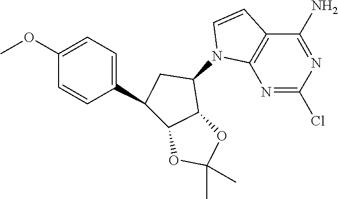 [SMILES: COc1ccc([C@H]2C[C@@H](n3ccc4c(N)nc(Cl)nc43)[C@@H]3OC(C)(C)O[C@@H]32)cc1]

Into an 8 mL vial were added 7-[(3aS,4R,6R,6aR)-6-(4-methoxyphenyl)-2,2-dimethyl-tetrahydro-3aH-cyclopenta[d][1,3]dioxol-4-yl]-2,4-dichloropyrrolo[2,3-d]pyrimidine 246g (0.12 g, 0.3 mmol, 1.0 eq) and 7M NH₃ in MeOH (2 mL) at room temperature. The reaction was stirred for 8 h at 80° C., then concentrated. The residue was purified by silica gel column chromatography (1:1 Petroleum ether: AcOEt) to afford 7-[(3aS,4R,6R,6aR)-6-(4-methoxyphenyl)-2,2-dimethyl-tetrahydro-3aH-cyclopenta[d][1,3]dioxol-4-yl]-2-chloropyrrolo[2,3-d]pyrimidin-4-amine 247g (80 mg, 67%) as a white solid. LCMS (conditions XBridge C18, 50*3.0 mm, 3.5 μm, Mobile Phase A: Water/5 mM NH₄HCO₃, Mobile Phase B: Acetonitrile, 10-95% B-2 min, 1.50 mL/min): T_{R}=1.43 min; ES m/z [M+H]⁺: 415, 417.

### (1R,2S,3R,5R)-3-{4-Amino-2-chloropyrrolo[2,3-d]pyrimidin-7-yl}-5-(4-methoxyphenyl)cyclopentane-1,2-diol (682)

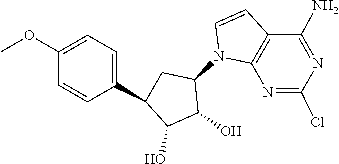 [SMILES: COc1ccc([C@H]2C[C@@H](n3ccc4c(N)nc(Cl)nc43)[C@H](O)[C@@H]2O)cc1]

Into an 8 mL vial were added 7-[(3aS,4R,6R,6aR)-6-(4-methoxyphenyl)-2,2-dimethyl-tetrahydro-3aH-cyclopenta[d][1,3]dioxol-4-yl]-2-chloropyrrolo[2,3-d]pyrimidin-4-amine 247g (80 mg, 0.2 mmol, 1.0 eq) and 4M HCl in MeOH (1.0 mL) at room temperature. The reaction was stirred for 0.5 h at room temperature, then concentrated. The residue was dissolved in MeOH (3 mL) and was basified to pH>8 with NH₃: H₂O. The residue was purified by Prep-HPLC [column, Xbridge Prep C18 OBD column, 5 μm, 19*150 mm; mobile phase, Water (0.03% NH₄OH) and CH₃CN (20-60% over 10 min); Detector, UV 220&254 nm]to afford (1R,2S,3R,5R)-3-{4-amino-2-chloropyrrolo[2,3-d]pyrimidin-7-yl}-5-(4-methoxyphenyl)cyclopentane-1,2-diol 682 (25.0 mg, 35%) as an off-white solid. LCMS (conditions: ZORBAX SB-Aq, 50*4.6 mm, 1.8 μm, Mobile Phase A: Water/0.02% TFA, Mobile Phase B: Acetonitrile, 5-95% B-3 min, 1.50 mL/min): T_{R}=2.11 min; ES m/z [M+H]⁺: 375, 377.1H NMR (300 MHZ, DMSO-d₆) δ 7.46 (s, 2H), 7.39 (d, J=3.5 Hz, 1H), 7.33 (d, J=8.7 Hz, 2H), 6.89 (d, J=8.7 Hz, 2H), 6.59 (d, J=3.5 Hz, 1H), 4.92 (d, J=6.0 Hz, 1H), 4.84 (d, J=6.3 Hz, 1H), 4.83-4.73 (m, 1H), 4.24 (q, J=6.5 Hz, 1H), 3.98 (q, J=6.4 Hz, 1H), 3.74 (s, 3H), 3.11-2.96 (m, 1H), 2.40-2.28 (m, 1H), 1.93 (q,)=11.6 Hz, 1H).

### Synthesis of (1R,2S,3R,5R)-3-(4-(methylamino)-7H-pyrrolo[2,3-d]pyrimidin-7-yl)-5-((1R,2S)-2-(3-(phenethylamino)propyl)cyclopropyl)cyclopentane-1,2-diol (683)

### (1R,2S,3R,5R)-3-(4-((4-methoxybenzyl)(methyl)amino)-7H-pyrrolo[2,3-d]pyrimidin-7-yl)-5-((E)-5-(phenethylamino) pent-1-en-1-yl)cyclopentane-1,2-diol (398d)

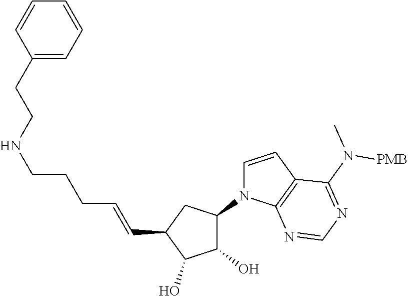

Into a 40 mL Vessel, tert-butyl N-{5-[(3aR,4R,6R,6aS)-6-(4-{[(4-methoxyphenyl)methyl](methyl)amino}pyrrolo[2,3-d]pyrimidin-7-yl)-2,2-dimethyl-tetrahydro-3aH-cyclopenta[d][1,3]dioxol-4-yl]pent-4-en-1-yl}-N-(2-phenylethyl)carbamate (90 mg, 0.129 mmol, 1.0 eq) was dissolved into DCM (4.0 mL). Then TFA (1.0 mL) was added at 0° C. The resulting mixture was stirred for 2 h at room temperature. The resulting mixture was concentrated under reduced pressure. The crude residue product was purified by reverse flash chromatography with the following conditions (column, C18; mobile phase, MeOH in Water/0.05% ammonia water, 50% to 90% gradient in 15 min; detector, UV 220 nm) to afford (1R,2S,3R,5R)-3-(4-{[(4-methoxyphenyl)methyl](methyl)amino}pyrrolo[2,3-d]pyrimidin-7-yl)-5-{5-[(2-phenylethyl)amino]pent-1-en-1-yl}cyclopentane-1,2-diol 398d (56 mg, 78%) as a light brown oil. LCMS (conditions: XBridge C18, 50*3.0 mm, 3.5 μm, Mobile Phase A: Water/5 mM NH₄HCO₃, Mobile Phase B: Methanol, 10%-95% B-3 min, 1.50 mL/min, ES, m/z): T_{R}=2.10 min; [M+H]⁺: 556.

### (1R,2S,3R,5R)-3-(4-((4-methoxybenzyl)(methyl)amino)-7H-pyrrolo[2,3-d]pyrimidin-7-yl)-5-((1R,2S)-2-(3-(phenethylamino)propyl)cyclopropyl)cyclopentane-1,2-diol (399d)

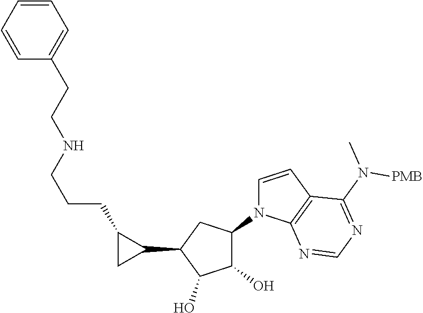

Into a 8 mL Vessel was added diethylzinc (0.505 ml, 1M in hexane, 0.505 mmol, 5.0 eq), the solution was cooled to 0° C., CH₂₁₂ (134.95 mg, 0.505 mmol, 5.0 eq) was added dropwise. The mixture was stirred at 0° C. for 30 min. (1R,2S,3R,5R)-3-(4-{[(4-methoxyphenyl)methyl](methyl)amino}pyrrolo[2,3-d]pyrimidin-7-yl)-5-{5-[(2-phenylethyl)amino]pent-1-en-1-yl}cyclopentane-1,2-diol 398d (56 mg, 0.101 mmol, 1.0 eq) in DCE (5.0 mL) was added dropwise. The reaction mixture was warm to room temperature and stirred at that temperature for 2 h. The resulting mixture was concentrated under reduced pressure. The crude product was purified by reverse flash chromatography with the following conditions (column, C18 silica gel; mobile phase, MeOH in Water/0.05% ammonia water, 50% to 90% gradient in 15 min; detector, UV 220 nm) to afford (1R,2S,3R,5R)-3-(4-{[(4-methoxyphenyl)methyl](methyl)amino}pyrrolo[2,3-d]pyrimidin-7-yl)-5-[(25)-2-{3-[(2-phenylethyl)amino]propyl}cyclopropyl]cyclopentane-1,2-diol 399d (24 mg, 38%) as a white solid. LCMS (conditions: XBridge C18, 50*3.0 mm, 3.5 μm, Mobile Phase A: Water/5 mM NH₄HCO₃, Mobile Phase B: Methanol, 10%-95% B-3 min, 1.50 mL/min, ES, m/z): T_{R}=2.07 min; [M+H]⁺: 570.

### (1R,2S,3R,5R)-3-(4-(methylamino)-7H-pyrrolo[2,3-d]pyrimidin-7-yl)-5-((1R,2S)-2-(3-(phenethylamino)propyl)cyclopropyl)cyclopentane-1,2-diol/622)

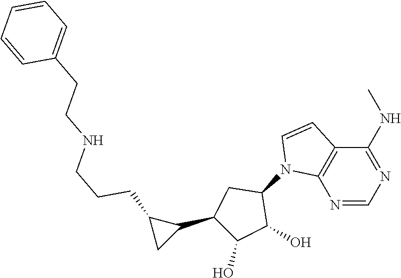 [SMILES: CNc1ncnc2c1ccn2[C@@H]1C[C@H](C2C[C@@H]2CCCNCCc2ccccc2)[C@@H](O)[C@H]1O]

Into a 8 MI Vessel were added trifluoroacetic acid (1 mL) and (1R,2S,3R,5R)-3-(4-{[(4-methoxyphenyl)methyl](methyl)amino}pyrrolo[2,3-d]pyrimidin-7-yl)-5-[(25)-2-{3-[(2-phenylethyl)amino]propyl}cyclopropyl]cyclopentane-1,2-diol 399d (24 mg, 0.038 mmol, 1.0 eq) at 0° C. The solution was stirred for 16 h. The resulting mixture was concentrated under vacuum. The residue was dissolved in DMF (2 mL) and basified to pH>8 with NH₃·H₂O. The mixture was purified by Prep-HPLC with the following conditions (Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 30 mL/min; Gradient: 24% B to 49% B in 7 min, 49% B; Wave Length: 220 nm) to afford (1R,2S,3R,5R)-3-[4-(methylamino)pyrrolo[2,3-d]pyrimidin-7-yl]-5-[(25)-2-{3-[(2-phenylethyl)amino]propyl}cyclopropyl]cyclopentane-1,2-diol 683 (2.1 mg, 11%) as an off-white solid. ¹H NMR (400 MHZ, Chloroform-d) 88.29 (s, 1H), 7.34-7.27 (m, 3H), 7.24-7.18 (m, 3H), 6.97 (d, J=3.6 Hz, 1H), 6.37 (d, J=3.6 Hz, 1H), 4.78-4.60 (m, 2H), 4.30-4.21 (m, 1H), 4.17-4.10 (m, 1H), 3.20 (s, 3H), 2.96-2.76 (m, 4H), 2.72-2.56 (m, 3H), 2.00-1.86 (m, 1H), 1.78-1.67 (m, 1H), 1.66-1.47 (m, 2H), 1.33-1.24 (m, 1H), 1.22-1.11 (m, 1H), 0.92-0.65 (m, 3H), 0.16-0.08 (m, 1H). LCMS (conditions: HPH-C18, 50*3.0 mm, 2.7 μm, Mobile Phase A: Water/0.05% ammonia water, Mobile Phase B: Acetonitrile, 5%-50%-95% B-7.0 min, 1.20 mL/min, ES, m/z): T_{R}=4.438 min; [M+H]⁺: 450.

### 2. Testing of the Compounds for KMT9-Inhibition and KMT9-Binding

### Microscale Thermophoresis (MST) to Test KMT9-Binding by Compounds of the Present Invention

To determine the binding affinity of the compounds according to the present invention and KMT9, microscale thermophoresis (MST) analysis was performed with a NanoTemper Monolith NT.115 instrument (NanoTemper Technologies GmbH). KMT9 was labelled with a RED-Tris-NTA labelling kit (NanoTemper Technologies GmbH) based on the manufacturer's instructions. Buffer including 25 mM HEPES (pH 7.5), 100 mM NaCl, 1 mM DTT and 0.05% Tween was used for the reaction buffer. Varying concentrations of the tested compounds of formula (I) as indicated in Table 1 were titrated against His-tag labelled KMT9 proteins (20 nM). Samples were loaded into standard Capillaries (NanoTemper Technologies GmbH) and MST measurements were performed using 40% MST power and 100% LED power. For each set of binding experiments, MST measurement was carried out with Binding Affinity module in MO.Control program under Nano-RED Excitation. Datasets were processed with the MO.Affinity Analysis software (NanoTemper Technologies GmbH).

For the results of the microscale thermophoresis (MST) assay shown as “MST” in Table 1, “A” represents a K_{d} of equal to or less than 10 nM (K_{d}≤10 nM); “B” represents a K_{d} of greater than 10 nM and equal to or less than 100 nM (10 nM<K_{d}<100 nM); “C” represents a K_{d} of greater than 100 nM and equal to or less than 1000 nM (100 nM<K_{d} ≤ 1000 nM); “D” represents a K_{d} of greater than 1000 nM (1000 nM<K_{d}).

### KMT9-Thermal Shift Assay to Test the Increase in Thermal Stability of KMT9 Upon Inhibitor Binding

The assay was carried out in 96-well Hard-Shell PCR plates (Bio-Rad Laboratories Inc.). Assay buffer consisted of 50 mM BTP, 1 mM MgCl₂ and 1 mM DTT at a pH of 8.5 (adjusted using 1 M HCl and 1 M NaOH). 20X inhibitor stock (in DMSO), 2X enzyme stock and 4X SyproOrange (SO) stock (both in assay buffer) were prepared. Final assay concentrations were set to 500 UM of the tested compound of formula (I) as indicated in Table 1, 2 μM KMT9 and 5X SO. 1 μL of 20X compound, 4 μL of assay buffer, 10 μL of 2X KMT9 and 5 μL of 4X SO were pipetted. For the no-compound control, 20X inhibitor stock was replaced by DMSO. The final volume was 20 μL. The plate was spun down at 700 rpm for 1 min using a Hettich UNIVERSAL 320 centrifuge and incubated at 25° C., shaking at 600 rpm. After incubation, the plate was spun down for 1 min at 700 rpm and measurement was conducted using a CFX96 Touch Real-Time PCR Detection System (Bio-Rad Laboratories Inc.). The plate was equilibrated at 20° C. for 4 min and then heated stepwise at a rate of 1° C. per 15 s until 95° C. After every step, fluorescence was measured in FRET mode. Calculation of melting points was conducted by a Boltzman sigmoidal model using GraphPad Prism 7.0.

For the results of the thermal shift assay shown as “Thermal shift” in Table 1, “++++” represents a ΔTₘ of greater 20 K, “+++” represents a ΔTₘ of greater than 15 K and less than or equal to 20 K (15 K<ΔTₘ≤20 K); “++” represents a ΔTₘ of greater than 10 K and less than or equal to 15 K (10 K<ΔTₘ ≤ 15 K); “+” represents a ΔTₘ of equal to or less than 10 K (ΔTₘ≤10 K).

### Inhibition of Cell Proliferation (“In Vivo”)

Cell proliferation was determined using X-Celligence RTCA system (Roche). For real-time recording of HT-1376, resp. Cal29 cell proliferation, 5000 cells/well were seeded in 16 well E-plates (Roche). Cells were seeded in the presence of DMSO or the tested compound of formula (I) as indicated in Table 124 h before seeding in E-plates. Cell indices were automatically recorded every 15 minutes. Relative velocities represent the change of the cell index over time.

For the results of the HT-1376, resp. Cal29 cell proliferation assay shown as “in vivo (HT-1376 Cells)” and “in vivo (Cal29 Cells)” in Table 1, “***” represents an anti-proliferation effect at less than or equal to 10 μM; “**” represents an anti-proliferation effect at greater than 10 UM and less than or equal to 30 μM; “*” represents an anti-proliferation effect at greater than 30 UM and less than or equal to 100 μM.

Inhibition of cell viability (“in vivo (MTT, PC3M cells)”)

MTT assays were performed with the CellTiter 96 Non-Radioactive Cell Proliferation Assay (MTT) kit (G4000, Promega) according to the manufacturer's instructions. Briefly, 100 μl of cell culture medium containing inhibitor were dispensed per well into 96-well tissue culture dishes and allowed to equilibrate for 30 min at room temperature. 100 μl of cell suspension were added per well (resulting in 300 cells per well). Cells were allowed to adhere for 30 min at room temperature and then incubated at 37° C. (5% CO₂) in a CellXpert incubator (Eppendorf). After 4 days, the medium was replaced with 100 μl of fresh cell culture medium containing inhibitor followed by incubation for additional 3 days. At the end of the culture period, 15 μl of Dye Solution per well were added, the plate incubated for 4 hours at 37° C. followed by addition of 100 μl of Solubilization Solution/Stop Mix per well and incubation for 1 hour at 37° C. Finally, the content of the 96-well dish was homogenized by pipetting up and down and assayed at 570 nm and 650 nm using a NanoQuant Infinite M200 Pro plate reader (TECAN). Absorption at 650 nm (background) was substracted from absorption at 570 nm and the resulting values were normalized to those of the DMSO control.

For the results of the cell viability assay shown as “in vivo (MTT, PC3M Cells)” in Table 1 “###” represents an effect at less than or equal to 1 μM; “##” represents an effect at greater than 1 μM and less than or equal to 3 μM; “#” represents an effect at greater than 3 μM.

### KMT9 Methyltransferase Inhibition Assay (“Inhibition”)

For the compounds of entries 1-154 of Table 1 below, the following procedure was used to determine IC₅₀ values:

The assay was carried out in 0.5 mL Eppendorf tubes in duplicates. Assay buffer consisted of 50 mM BTP, 1 mM MgCl₂, 1 mM DTT and 0.01% Triton-X¹⁰⁰ at a pH of 8.5 (adjusted using 1 M HCl and 1 M NaOH). 20X inhibitor stock (in DMSO), 2X enzyme stock and 4X substrate mix (both in assay buffer) were prepared. The substrate mix consisted of Tritium-labelled S-adenosylmethionine (3H-SAM), S-adenosylmethionine (SAM) and eukaryotic peptide chain release factor subunit1 (Homo sapiens, residues 140-275)(ETF1). Final assay concentrations were set to 25 nM KMT9, 0.3 μM 3H-SAM, 0.7 UM SAM and 5 μM ETF1. A 1:1 dilution series of 20x stocks of the tested compound of formula (I) as indicated in Table 1 (10 concentrations) was prepared in an optimized range of 250-0,488 M. 1 μL of the appropriate 20× stock of the tested compound of formula (I) as indicated in Table 1, 4 μL of assay buffer and 10 μL of 2x KMT9 stock were pipetted and incubated at 25° C. for 15 minutes. 5 μL of 4x substrate mix was added to initiate the reaction. The final reaction volume was 20 μL. For the positive control, the compound of formula (I) was replaced by DMSO; for the negative control, the compound of formula (I) was replaced by DMSO and KMT9 was replaced by assay buffer. The reaction mix was incubated in an Eppendorf thermomixer comfort at 30° C. for 2 h, shaking at 300 rpm. The reaction was quenched using 5 μL 50% trichloroacetic acid (TCA) and incubated at 25° C. for 5 minutes. 22 μL of the mixture was transferred into 96-well MultiScreen@HTS FB filter plates (Merck KGaA, Darmstadt, Germany) and washed four times with 10% TCA and two times with 100% ethanol. The plate was dried overnight, the filters were transferred into individual 6 mL Pony Vials (Perkin Elmer Inc, Waltham, MA, USA) and incubated in 3 mL of Ultima Gold scintillation cocktail (Perkin Elmer Inc, Waltham, MA, USA) for 30 minutes. The scintillation signal was measured for 3x 1 min using TriCarb 2910 T_{R} (Perkin Elmer) scintillation counter in 3H CPM mode (LL: 0, UL: 18.6). Inhibition was calculated using the following formula:

$$Inhibition [ \% ] = ( 1 - \frac{{x}_{c} - {x}_{p o s}}{{x}_{p o s} - {x}_{n e g}} ) * 100$$

With x_{c}: signal of compound, Xₚₒₛ: mean signal of positive control, X_{neg}: mean signal of negative control. Data fitting was carried out by GraphPad 7.0 using nonlinear fit ([Inhibitor]vs. response-variable slope (four parameters)).

For the compounds from entry 155 on in Table 1, the following procedure was used to determine IC₅₀ values: The assay was carried out in white, opaque 384-well OptiPlates (PerkinElmer). For IC₅₀ determination, a 2.5x substrate mix (final assay concentrations: 5 μM ETF1, 1 μM SAM and 1x MTaseGlo Reagent), a 2.5x KMT9 solution (final assay concentration 100 nM) and a 5x inhibitor stock 1:1 dilution series (concentration according to the desired range; dilution series was prepared in assay buffer containing 10% DMSO to obtain a final DMSO concentration of 2% in the assay) were prepared. 2 μL of 5x inhibitor stock and 4 μL of 2.5x KMT9 were pipetted, the plate was spun down at 700 rpm for 1 min using a Hettich UNIVERSAL 320 centrifuge and incubated at room temperature (around 25° C.) for 15 min. The reaction was initiated by adding 4 μL of 2.5x substrate mix, the plate was spun down as described before and incubated at 30° C. for 2 h. Afterwards, 10 μL of 2× MTaseGlo Detection solution was added, the plate was centrifuged as described above and incubated for 60 min at room temperature. Luminescence was measured using a PerkinElmer EnVision 2102 Multilabel Reader. Calculation of inhibition and data fitting was carried out as for entries 1-154. described above.

For the results of the methyltransferase inhibition assay shown as “Inhibition” in Table 1, “I” represents an IC₅₀ less than 0.1 μM, “II” represents an IC₅₀ equal or higher than 0.1 μM and less than 1 μM, “III” represents an IC₅₀ equal or higher than 1 UM and less than 10 UM and “IV” represents an IC₅₀ equal or higher than 10 UM

TABLE 1

| | | | | | | In | In | In |
|---|---|---|---|---|---|---|---|---|
| | | | | | in- | vivo | vivo | vivo |
| | | | | Ther- | hi- | (HT- | (Cal- | (MTT, |
| En- | | | | mal | bi- | 1376 | 29 | PC3M |
| try | Name | Structure | MST | shift | tion | Cells) | Cells) | Cells) |
| 1 | 287 (184) | 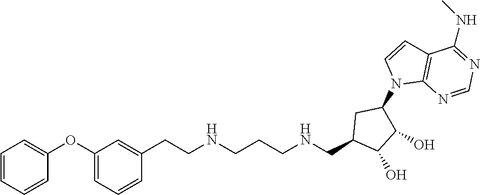 [SMILES: CNc1ncnc2c1ccn2[C@@H]1C[C@H](CNCCCNCCc2cccc(Oc3ccccc3)c2)[C@@H](O)[C@H]1O] | C | ++ | III | *** | NA | NA |
| 2 | 289 (186) | 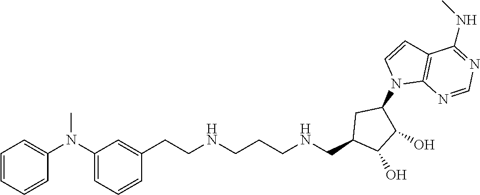 [SMILES: CNc1ncnc2c1ccn2[C@@H]1C[C@H](CNCCCNCCc2cccc(N(C)c3ccccc3)c2)[C@@H](O)[C@H]1O] | D | ++ | IV | *** | NA | NA |
| 3 | 291 (188) | 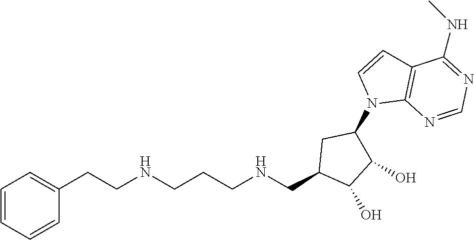 [SMILES: CNc1ncnc2c1ccn2[C@@H]1C[C@H](CNCCCNCCc2ccccc2)[C@@H](O)[C@H]1O] | D | ++ | III | ** | NA | NA |
| 4 | 293 (190) | 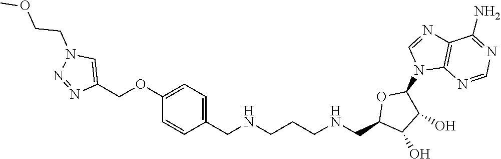 [SMILES: COCCn1cc(COc2ccc(CNCCCNC[C@H]3O[C@@H](n4cnc5c(N)ncnc54)[C@H](O)[C@@H]3O)cc2)nn1] | D | + | IV | NA | NA | NA |
| 5 | 295 (192) | 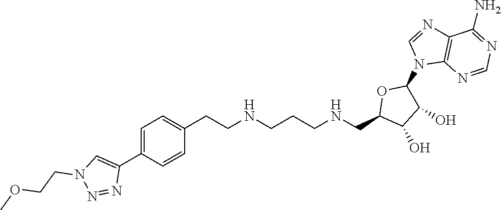 [SMILES: COCCn1cc(-c2ccc(CCNCCCNC[C@H]3O[C@@H](n4cnc5c(N)ncnc54)[C@H](O)[C@@H]3O)cc2)nn1] | C | + | IV | NA | NA | NA |
| 6 | 297 (194) | 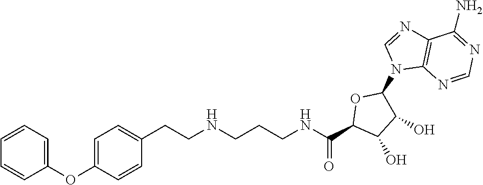 [SMILES: Nc1ncnc2c1ncn2[C@@H]1O[C@H](C(=O)NCCCNCCc2ccc(Oc3ccccc3)cc2)[C@@H](O)[C@H]1O] | C | + | III | NA | NA | NA |
| 7 | 299 (196) | 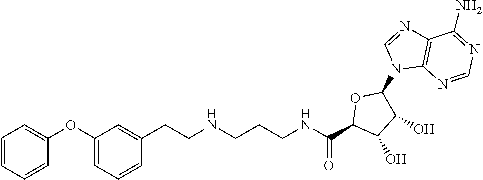 [SMILES: Nc1ncnc2c1ncn2[C@@H]1O[C@H](C(=O)NCCCNCCc2cccc(Oc3ccccc3)c2)[C@@H](O)[C@H]1O] | C | ++ | III | NA | NA | NA |
| 8 | 301 (198) | 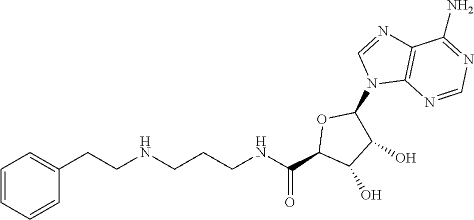 [SMILES: Nc1ncnc2c1ncn2[C@@H]1O[C@H](C(=O)NCCCNCCc2ccccc2)[C@@H](O)[C@H]1O] | C | + | IV | NA | NA | NA |
| 9 | 309 (208) | 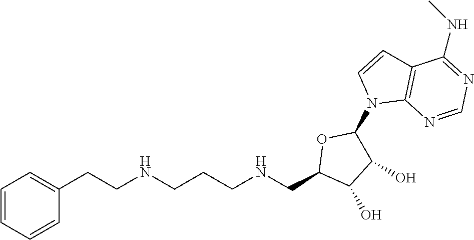 [SMILES: CNc1ncnc2c1ccn2[C@@H]1O[C@H](CNCCCNCCc2ccccc2)[C@@H](O)[C@H]1O] | D | ++ | IV | NA | NA | NA |
| 10 | 303 (200) | 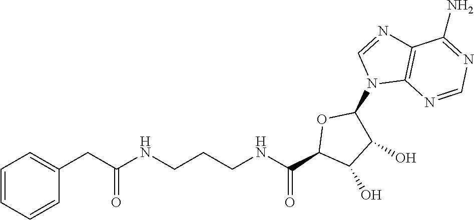 [SMILES: Nc1ncnc2c1ncn2[C@@H]1O[C@H](C(=O)NCCCNC(=O)Cc2ccccc2)[C@@H](O)[C@H]1O] | D | + | IV | NA | NA | NA |
| 11 | 684 (202) | 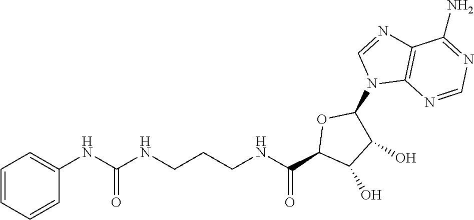 [SMILES: Nc1ncnc2c1ncn2[C@@H]1O[C@H](C(=O)NCCCNC(=O)Nc2ccccc2)[C@@H](O)[C@H]1O] | D | + | IV | NA | NA | NA |
| 12 | 305 (204) | 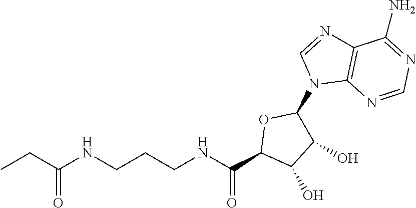 [SMILES: CCC(=O)NCCCNC(=O)[C@H]1O[C@@H](n2cnc3c(N)ncnc32)[C@H](O)[C@@H]1O] | D | + | IV | NA | NA | NA |
| 13 | 307 (206) | 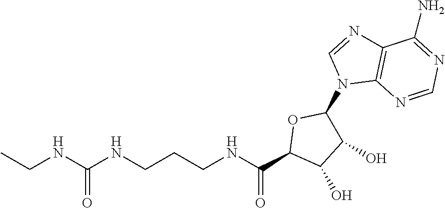 [SMILES: CCNC(=O)NCCCNC(=O)[C@H]1O[C@@H](n2cnc3c(N)ncnc32)[C@H](O)[C@@H]1O] | D | + | IV | NA | NA | NA |
| 14 | 311 (210) | 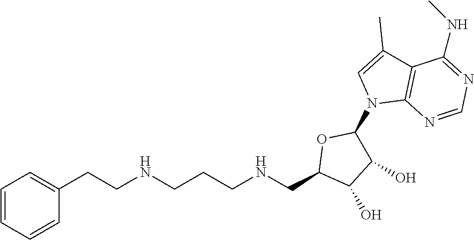 [SMILES: CNc1ncnc2c1c(C)cn2[C@@H]1O[C@H](CNCCCNCCc2ccccc2)[C@@H](O)[C@H]1O] | D | + | IV | NA | NA | NA |
| 15 | 313 (212) | 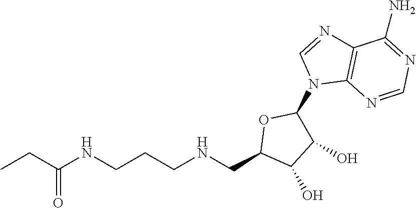 [SMILES: CCC(=O)NCCCNC[C@H]1O[C@@H](n2cnc3c(N)ncnc32)[C@H](O)[C@@H]1O] | D | + | IV | NA | NA | NA |
| 16 | 686 (214) | 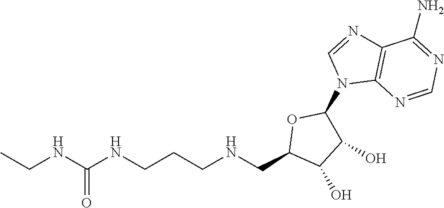 [SMILES: CCNC(=O)NCCCNC[C@H]1O[C@@H](n2cnc3c(N)ncnc32)[C@H](O)[C@@H]1O] | D | + | IV | NA | NA | NA |
| 17 | 315 (216) | 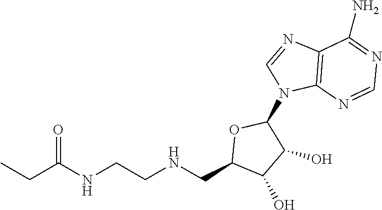 [SMILES: CCC(=O)NCCNC[C@H]1O[C@@H](n2cnc3c(N)ncnc32)[C@H](O)[C@@H]1O] | D | + | IV | NA | NA | NA |
| 18 | 317 (218) | 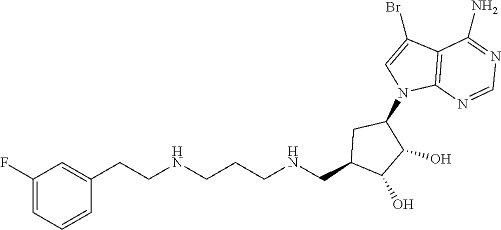 [SMILES: Nc1ncnc2c1c(Br)cn2[C@@H]1C[C@H](CNCCCNCCc2cccc(F)c2)[C@@H](O)[C@H]1O] | B | +++ | II | ** | NA | NA |
| 19 | 319 (220) | 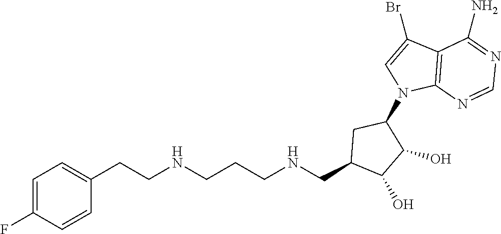 [SMILES: Nc1ncnc2c1c(Br)cn2[C@@H]1C[C@H](CNCCCNCCc2ccc(F)cc2)[C@@H](O)[C@H]1O] | B | +++ | II | ** | NA | NA |
| 20 | 416 (287) | 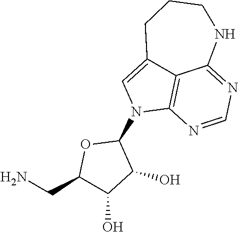 [SMILES: NC[C@H]1O[C@@H](n2cc3c4c(ncnc42)NCCC3)[C@H](O)[C@@H]1O] | D | + | IV | NA | NA | NA |
| 21 | 346 (250) | 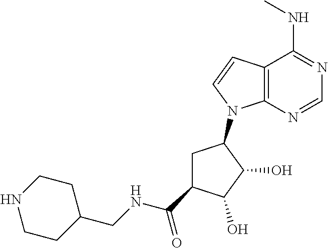 [SMILES: CNc1ncnc2c1ccn2[C@@H]1C[C@H](C(=O)NCC2CCNCC2)[C@@H](O)[C@H]1O] | D | + | IV | NA | NA | NA |
| 22 | 348 (252) | 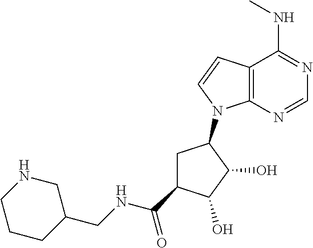 [SMILES: CNc1ncnc2c1ccn2[C@@H]1C[C@H](C(=O)NCC2CCCNC2)[C@@H](O)[C@H]1O] | D | + | IV | NA | NA | NA |
| 23 | 350 (254) | 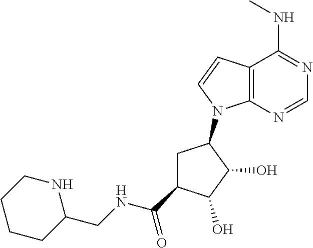 [SMILES: CNc1ncnc2c1ccn2[C@@H]1C[C@H](C(=O)NCC2CCCCN2)[C@@H](O)[C@H]1O] | D | + | IV | NA | NA | NA |
| 24 | 353 (257) | 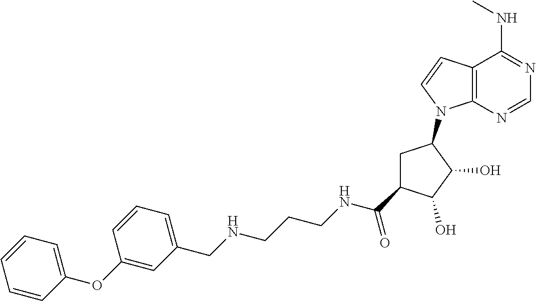 [SMILES: CNc1ncnc2c1ccn2[C@@H]1C[C@H](C(=O)NCCCNCc2cccc(Oc3ccccc3)c2)[C@@H](O)[C@H]1O] | C | ++ | III | NA | NA | NA |
| 25 | 352 (256) | 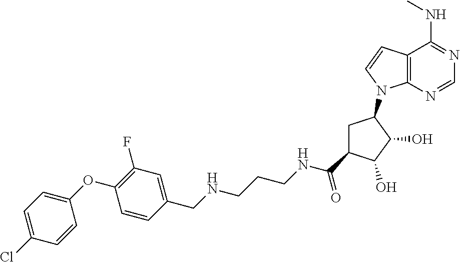 [SMILES: CNc1ncnc2c1ccn2[C@@H]1C[C@H](C(=O)NCCCNCc2ccc(Oc3ccc(Cl)cc3)c(F)c2)[C@@H](O)[C@H]1O] | C | ++ | IV | NA | NA | NA |
| 26 | 355 (259) | 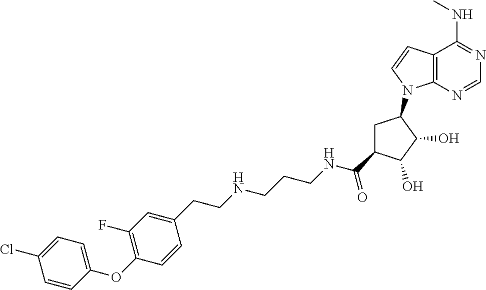 [SMILES: CNc1ncnc2c1ccn2[C@@H]1C[C@H](C(=O)NCCCNCCc2ccc(Oc3ccc(Cl)cc3)c(F)c2)[C@@H](O)[C@H]1O] | C | ++ | II | NA | NA | NA |
| 27 | 354 (258) | 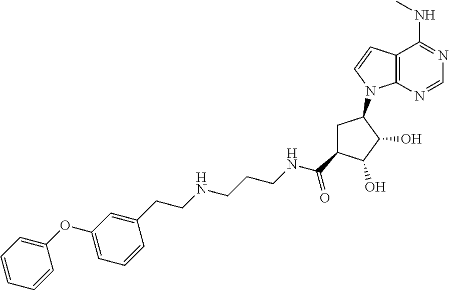 [SMILES: CNc1ncnc2c1ccn2[C@@H]1C[C@H](C(=O)NCCCNCCc2cccc(Oc3ccccc3)c2)[C@@H](O)[C@H]1O] | B | +++ | III | * | NA | NA |
| 28 | 351 (255) | 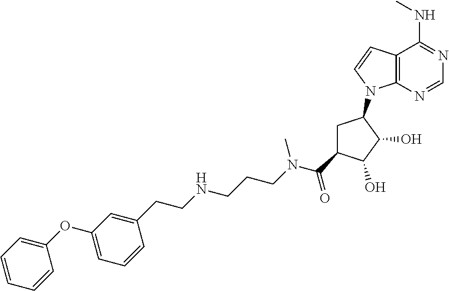 [SMILES: CNc1ncnc2c1ccn2[C@@H]1C[C@H](C(=O)N(C)CCCNCCc2cccc(Oc3ccccc3)c2)[C@@H](O)[C@H]1O] | B | ++ | III | ** | NA | NA |
| 29 | 356 (260) | 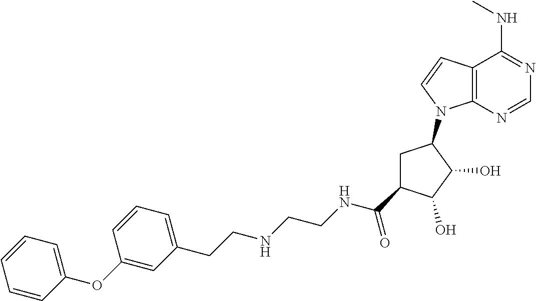 [SMILES: CNc1ncnc2c1ccn2[C@@H]1C[C@H](C(=O)NCCNCCc2cccc(Oc3ccccc3)c2)[C@@H](O)[C@H]1O] | C | ++ | IV | ** | NA | NA |
| 30 | 357 (261) | 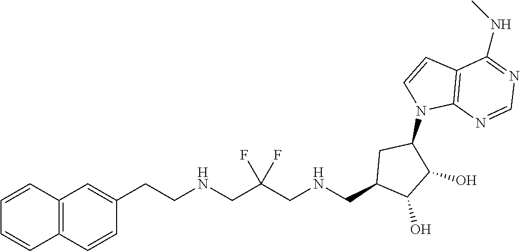 [SMILES: CNc1ncnc2c1ccn2[C@@H]1C[C@H](CNCC(F)(F)CNCCc2ccc3ccccc3c2)[C@@H](O)[C@H]1O] | D | + | IV | NA | NA | NA |
| 31 | 359 (263) | 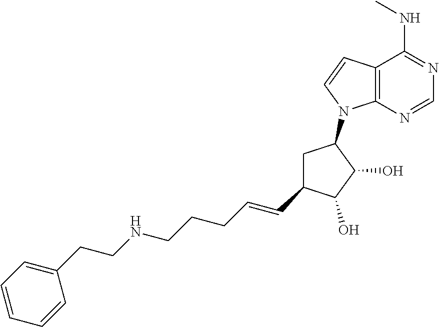 [SMILES: CNc1ncnc2c1ccn2[C@@H]1C[C@H](/C=C/CCCNCCc2ccccc2)[C@@H](O)[C@H]1O] | D | + | IV | NA | NA | NA |
| 32 | 360 (264) | 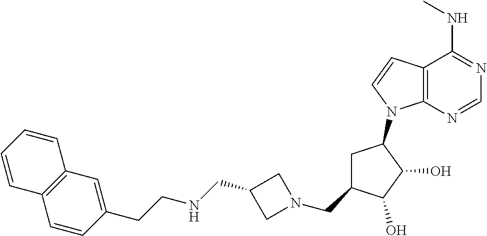 | D | + | IV | *** | NA | NA |
| 33 | 362 (266) | 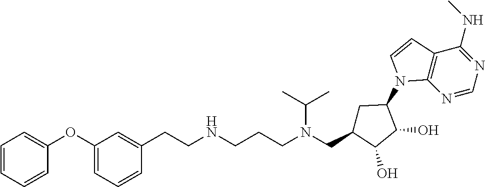 [SMILES: CNc1ncnc2c1ccn2[C@@H]1C[C@H](CN(CCCNCCc2cccc(Oc3ccccc3)c2)C(C)C)[C@@H](O)[C@H]1O] | C | ++ | III | ** | NA | NA |
| 34 | 364 (268) | 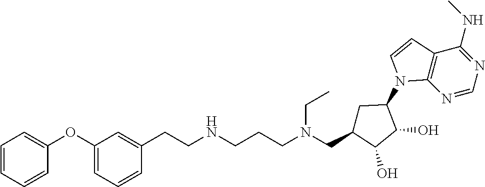 [SMILES: CCN(CCCNCCc1cccc(Oc2ccccc2)c1)C[C@H]1C[C@@H](n2ccc3c(NC)ncnc32)[C@H](O)[C@@H]1O] | C | ++ | III | *** | NA | NA |
| 35 | 367 (271) | 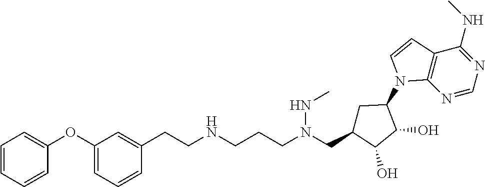 [SMILES: CNc1ncnc2c1ccn2[C@@H]1C[C@H](CN(CCCNCCc2cccc(Oc3ccccc3)c2)NC)[C@@H](O)[C@H]1O] | D | + | IV | NA | NA | NA |
| 36 | 360 (270) | 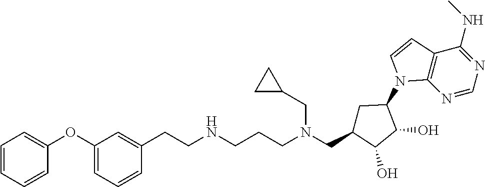 [SMILES: CNc1ncnc2c1ccn2[C@@H]1C[C@H](CN(CCCNCCc2cccc(Oc3ccccc3)c2)CC2CC2)[C@@H](O)[C@H]1O] | C | + | IV | *** | NA | NA |
| 37 | 368 (272) | 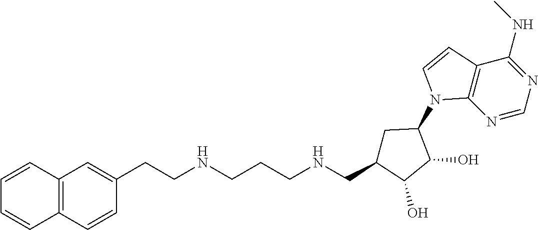 [SMILES: CNc1ncnc2c1ccn2[C@@H]1C[C@H](CNCCCNCCc2ccc3ccccc3c2)[C@@H](O)[C@H]1O] | D | ++ | IV | NA | NA | NA |
| 38 | 370 (274) | 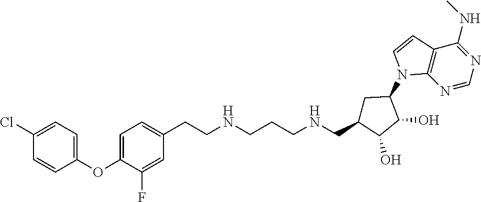 [SMILES: CNc1ncnc2c1ccn2[C@@H]1C[C@H](CNCCCNCCc2ccc(Oc3ccc(Cl)cc3)c(F)c2)[C@@H](O)[C@H]1O] | D | ++ | III | NA | NA | NA |
| 39 | 372 (276) | 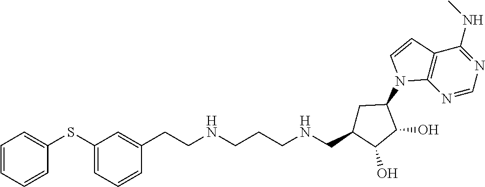 [SMILES: CNc1ncnc2c1ccn2[C@@H]1C[C@H](CNCCCNCCc2cccc(Sc3ccccc3)c2)[C@@H](O)[C@H]1O] | C | ++ | III | *** | NA | NA |
| 40 | 374 (278) | 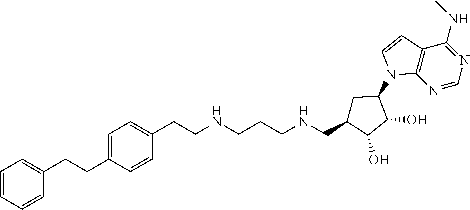 [SMILES: CNc1ncnc2c1ccn2[C@@H]1C[C@H](CNCCCNCCc2ccc(CCc3ccccc3)cc2)[C@@H](O)[C@H]1O] | D | ++ | IV | NA | NA | NA |
| 41 | 375 (279) | 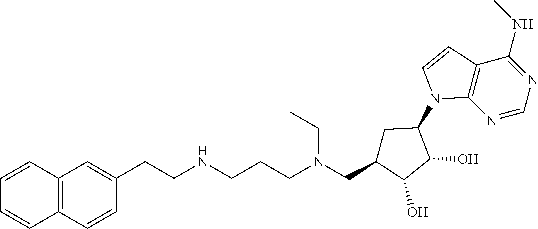 [SMILES: CCN(CCCNCCc1ccc2ccccc2c1)C[C@H]1C[C@@H](n2ccc3c(NC)ncnc32)[C@H](O)[C@@H]1O] | C | ++ | IV | ** | NA | NA |
| 42 | 377 (281) | 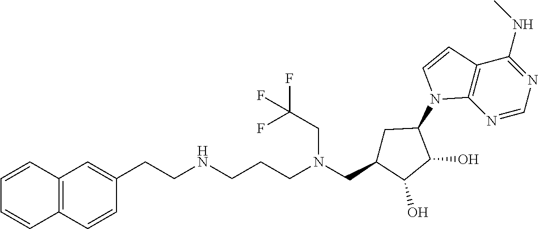 [SMILES: CNc1ncnc2c1ccn2[C@@H]1C[C@H](CN(CCCNCCc2ccc3ccccc3c2)CC(F)(F)F)[C@@H](O)[C@H]1O] | D | + | IV | ** | NA | NA |
| 43 | 326 (229) | 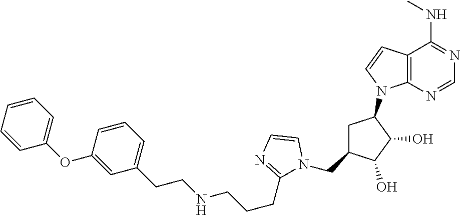 [SMILES: CNc1ncnc2c1ccn2[C@@H]1C[C@H](Cn2ccnc2CCCNCCc2cccc(Oc3ccccc3)c2)[C@@H](O)[C@H]1O] | D | + | IV | NA | NA | NA |
| 44 | 325 (228) | 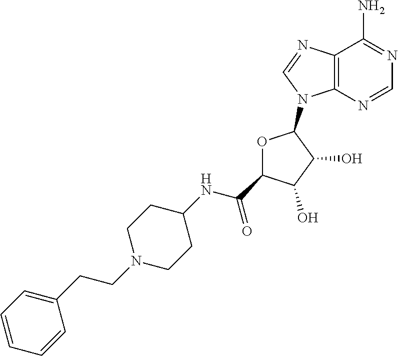 [SMILES: Nc1ncnc2c1ncn2[C@@H]1O[C@H](C(=O)NC2CCN(CCc3ccccc3)CC2)[C@@H](O)[C@H]1O] | D | + | IV | NA | NA | NA |
| 45 | 338 (242) | 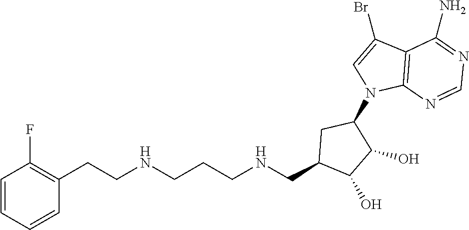 [SMILES: Nc1ncnc2c1c(Br)cn2[C@@H]1C[C@H](CNCCCNCCc2ccccc2F)[C@@H](O)[C@H]1O] | B | +++ | II | ** | NA | NA |
| 46 | 334 (238) | 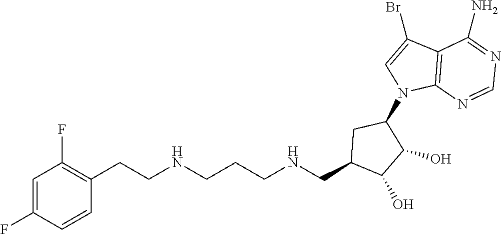 [SMILES: Nc1ncnc2c1c(Br)cn2[C@@H]1C[C@H](CNCCCNCCc2ccc(F)cc2F)[C@@H](O)[C@H]1O] | B | +++ | II | * | NA | NA |
| 47 | 336 (240) | 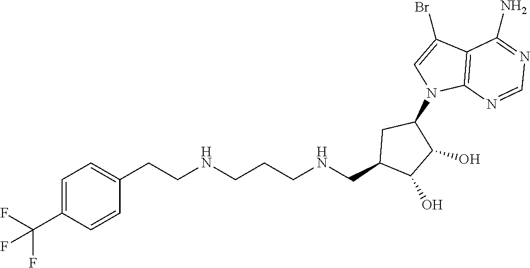 [SMILES: Nc1ncnc2c1c(Br)cn2[C@@H]1C[C@H](CNCCCNCCc2ccc(C(F)(F)F)cc2)[C@@H](O)[C@H]1O] | C | +++ | II | * | NA | NA |
| 48 | 330 (234) | 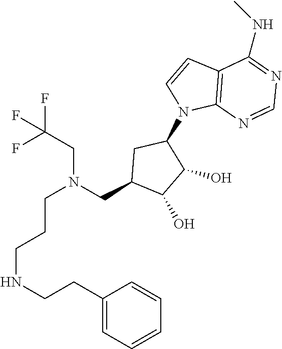 [SMILES: CNc1ncnc2c1ccn2[C@@H]1C[C@H](CN(CCCNCCc2ccccc2)CC(F)(F)F)[C@@H](O)[C@H]1O] | C | + | IV | * | NA | NA |
| 49 | 340 (244) | 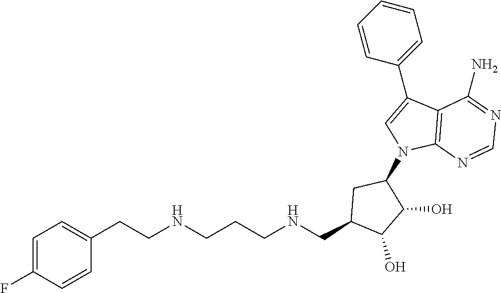 [SMILES: Nc1ncnc2c1c(-c1ccccc1)cn2[C@@H]1C[C@H](CNCCCNCCc2ccc(F)cc2)[C@@H](O)[C@H]1O] | B | NA | II | ** | NA | NA |
| 50 | 321 (224) | 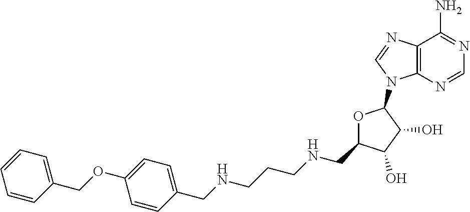 [SMILES: Nc1ncnc2c1ncn2[C@@H]1O[C@H](CNCCCNCc2ccc(OCc3ccccc3)cc2)[C@@H](O)[C@H]1O] | C | + | IV | NA | NA | NA |
| 51 | 323 (226) | 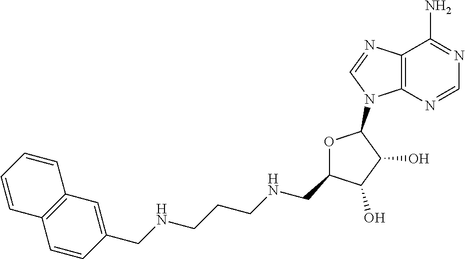 [SMILES: Nc1ncnc2c1ncn2[C@@H]1O[C@H](CNCCCNCc2ccc3ccccc3c2)[C@@H](O)[C@H]1O] | D | + | IV | NA | NA | NA |
| 52 | 688 (222) | 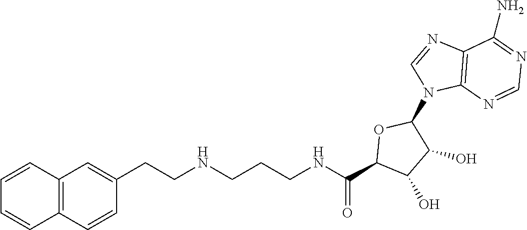 [SMILES: Nc1ncnc2c1ncn2[C@@H]1O[C@H](C(=O)NCCCNCCc2ccc3ccccc3c2)[C@@H](O)[C@H]1O] | | | IV | | | |
| 52 | 328 (232) | 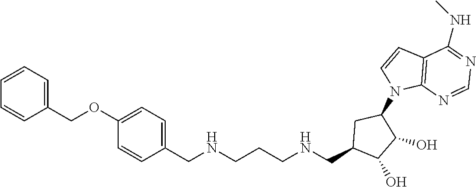 [SMILES: CNc1ncnc2c1ccn2[C@@H]1C[C@H](CNCCCNCc2ccc(OCc3ccccc3)cc2)[C@@H](O)[C@H]1O] | D | + | IV | NA | NA | NA |
| 53 | 332 (236) | 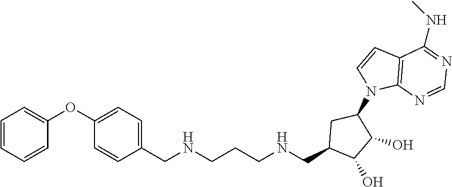 [SMILES: CNc1ncnc2c1ccn2[C@@H]1C[C@H](CNCCCNCc2ccc(Oc3ccccc3)cc2)[C@@H](O)[C@H]1O] | D | + | IV | NA | NA | NA |
| 54 | 343 (246) | 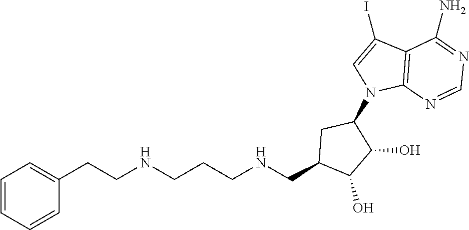 [SMILES: Nc1ncnc2c1c(I)cn2[C@@H]1C[C@H](CNCCCNCCc2ccccc2)[C@@H](O)[C@H]1O] | C | +++ | II | ** | NA | NA |
| 56 | 344 (248) | 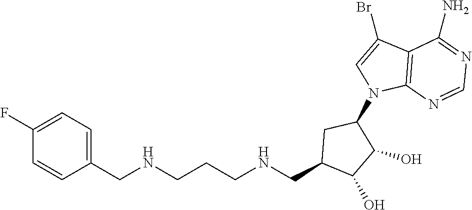 [SMILES: Nc1ncnc2c1c(Br)cn2[C@@H]1C[C@H](CNCCCNCc2ccc(F)cc2)[C@@H](O)[C@H]1O] | C | +++ | II | NA | NA | NA |
| 57 | 417 (288) | 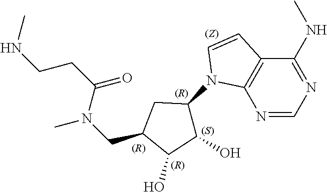 | D | + | IV | NA | NA | NA |
| 58 | 418 (289) | 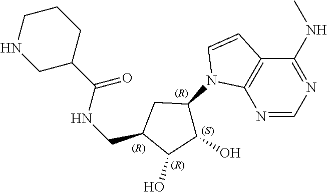 | D | + | IV | NA | NA | NA |
| 59 | 419 (290) | 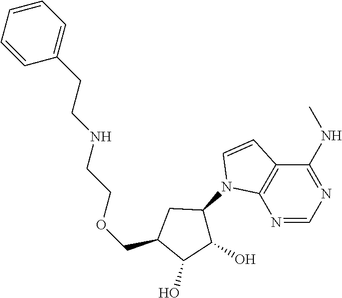 [SMILES: CNc1ncnc2c1ccn2[C@@H]1C[C@H](COCCNCCc2ccccc2)[C@@H](O)[C@H]1O] | D | + | IV | NA | NA | NA |
| 60 | 420 (291) | 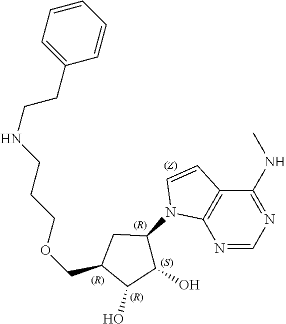 | D | + | IV | NA | NA | NA |
| 61 | 421 (292) | 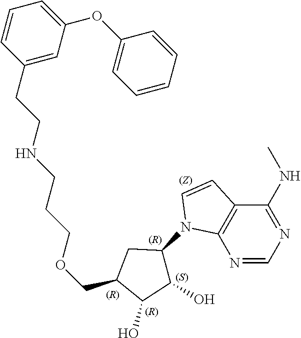 | D | + | IV | NA | NA | NA |
| 62 | 422 (293) | 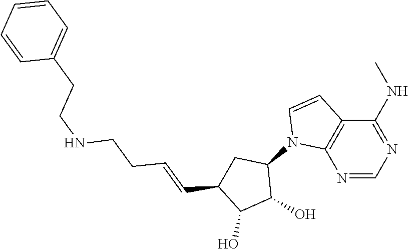 [SMILES: CNc1ncnc2c1ccn2[C@@H]1C[C@H](/C=C/CCNCCc2ccccc2)[C@@H](O)[C@H]1O] | D | + | IV | NA | NA | NA |
| 63 | 423 (294) | 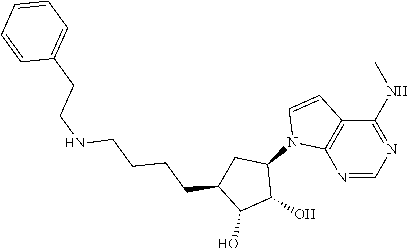 [SMILES: CNc1ncnc2c1ccn2[C@@H]1C[C@H](CCCCNCCc2ccccc2)[C@@H](O)[C@H]1O] | D | + | IV | NA | NA | NA |
| 64 | 424 (295) | 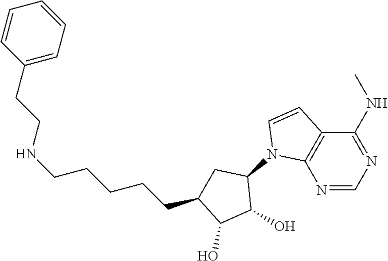 [SMILES: CNc1ncnc2c1ccn2[C@@H]1C[C@H](CCCCCNCCc2ccccc2)[C@@H](O)[C@H]1O] | C | ++ | III | NA | NA | NA |
| 65 | 425 (296) | 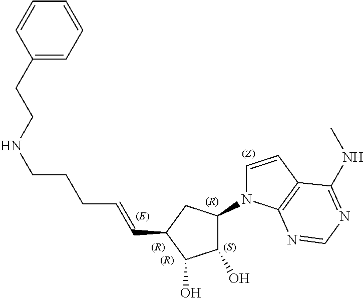 | D | + | IV | NA | NA | NA |
| 66 | 426 (297) | 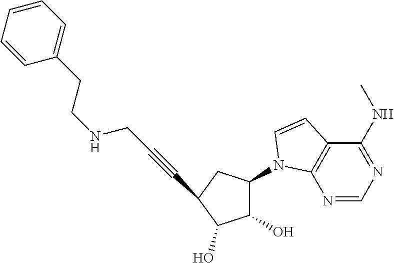 [SMILES: CNc1ncnc2c1ccn2[C@@H]1C[C@H](C#CCNCCc2ccccc2)[C@@H](O)[C@H]1O] | D | + | IV | NA | NA | NA |
| 67 | 689 (298) | 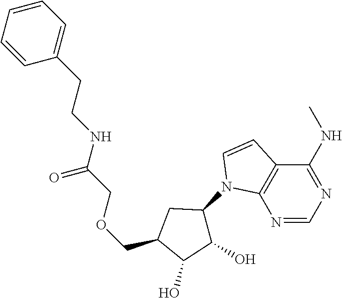 [SMILES: CNc1ncnc2c1ccn2[C@@H]1C[C@H](COCC(=O)NCCc2ccccc2)[C@@H](O)[C@H]1O] | D | + | IV | NA | NA | NA |
| 68 | 427 (299) | 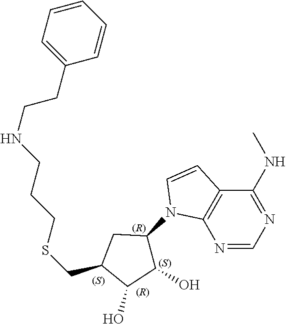 | D | + | III | NA | NA | NA |
| 69 | 428 (300) | 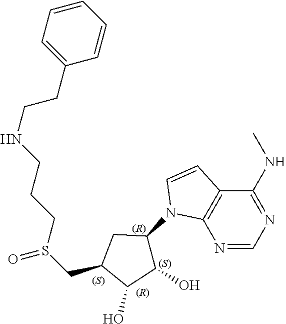 | D | + | IV | NA | NA | NA |
| 70 | 429 (301) | 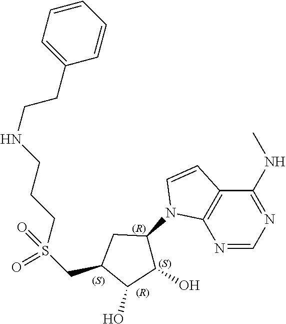 | D | + | IV | NA | NA | NA |
| 71 | 430 (302) | 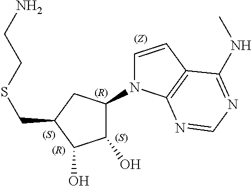 | D | + | IV | NA | NA | NA |
| 72 | 431 (303) | 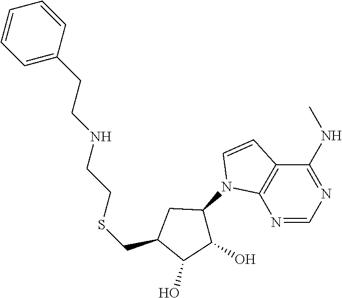 [SMILES: CNc1ncnc2c1ccn2[C@@H]1C[C@H](CSCCNCCc2ccccc2)[C@@H](O)[C@H]1O] | D | + | IV | NA | NA | NA |
| 73 | 432 (304) | 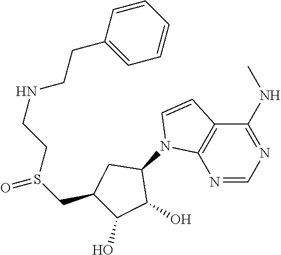 [SMILES: CNc1ncnc2c1ccn2[C@@H]1C[C@H](CS(=O)CCNCCc2ccccc2)[C@@H](O)[C@H]1O] | D | + | IV | NA | NA | NA |
| 74 | 433 (305) | 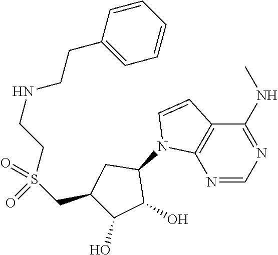 [SMILES: CNc1ncnc2c1ccn2[C@@H]1C[C@H](CS(=O)(=O)CCNCCc2ccccc2)[C@@H](O)[C@H]1O] | D | + | IV | NA | NA | NA |
| 75 | 434 (306) | 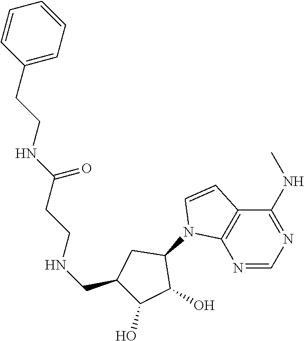 [SMILES: CNc1ncnc2c1ccn2[C@@H]1C[C@H](CNCCC(=O)NCCc2ccccc2)[C@@H](O)[C@H]1O] | D | + | IV | NA | NA | NA |
| | | 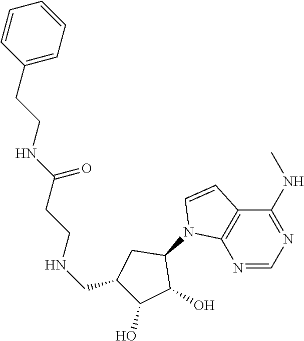 [SMILES: CNc1ncnc2c1ccn2[C@@H]1C[C@@H](CNCCC(=O)NCCc2ccccc2)[C@@H](O)[C@H]1O] | | | | | | |
| 76 | 435 (307) | 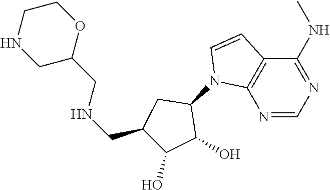 [SMILES: CNc1ncnc2c1ccn2[C@@H]1C[C@H](CNCC2CNCCO2)[C@@H](O)[C@H]1O] | D | + | IV | NA | NA | NA |
| | | 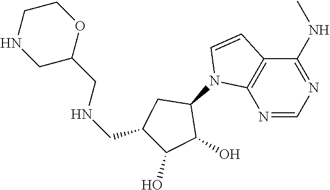 [SMILES: CNc1ncnc2c1ccn2[C@@H]1C[C@@H](CNCC2CNCCO2)[C@@H](O)[C@H]1O] | | | | | | |
| | | mixtures | | | | | | |
| 77 | 436 (308) | 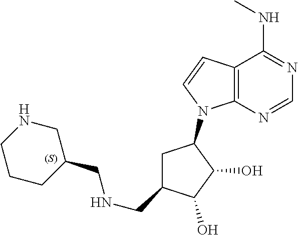 | D | + | IV | NA | NA | NA |
| 78 | 437 (309) | 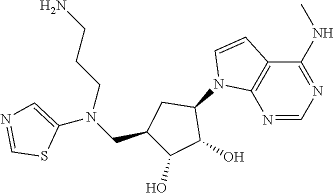 [SMILES: CNc1ncnc2c1ccn2[C@@H]1C[C@H](CN(CCCN)c2cncs2)[C@@H](O)[C@H]1O] | D | + | III | NA | NA | NA |
| 79 | 438 (310) | 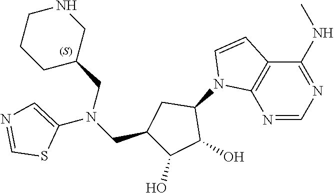 | D | + | IV | NA | NA | NA |
| 80 | 439 (311) | 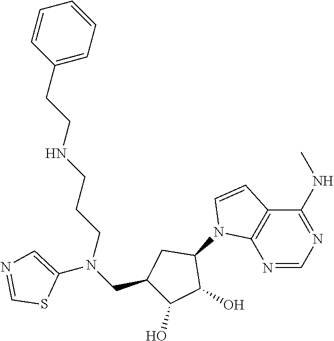 [SMILES: CNc1ncnc2c1ccn2[C@@H]1C[C@H](CN(CCCNCCc2ccccc2)c2cncs2)[C@@H](O)[C@H]1O] | D | + | III | NA | NA | NA |
| 81 | 440 (312) | 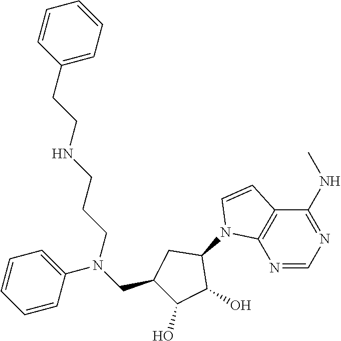 [SMILES: CNc1ncnc2c1ccn2[C@@H]1C[C@H](CN(CCCNCCc2ccccc2)c2ccccc2)[C@@H](O)[C@H]1O] | D | + | IV | NA | NA | NA |
| 82 | 441 (313) | 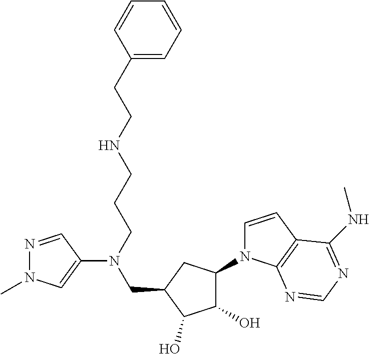 [SMILES: CNc1ncnc2c1ccn2[C@@H]1C[C@H](CN(CCCNCCc2ccccc2)c2cnn(C)c2)[C@@H](O)[C@H]1O] | D | + | IV | NA | NA | NA |
| 83 | 442 (314) | 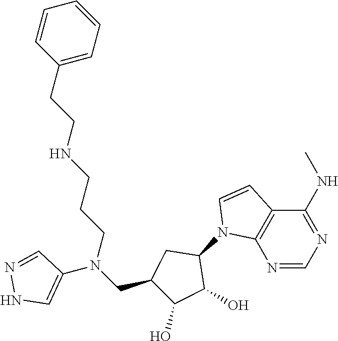 [SMILES: CNc1ncnc2c1ccn2[C@@H]1C[C@H](CN(CCCNCCc2ccccc2)c2cn[nH]c2)[C@@H](O)[C@H]1O] | C | ++ | III | NA | NA | NA |
| 84 | 443 (315) | 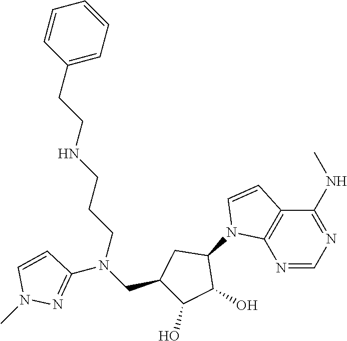 [SMILES: CNc1ncnc2c1ccn2[C@@H]1C[C@H](CN(CCCNCCc2ccccc2)c2ccn(C)n2)[C@@H](O)[C@H]1O] | D | + | IV | NA | NA | NA |
| 85 | 444 (316) | 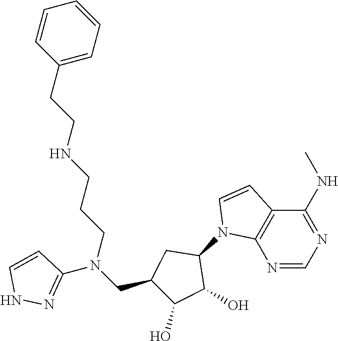 [SMILES: CNc1ncnc2c1ccn2[C@@H]1C[C@H](CN(CCCNCCc2ccccc2)c2cc[nH]n2)[C@@H](O)[C@H]1O] | D | + | III | NA | NA | NA |
| 86 | 445 (317) | 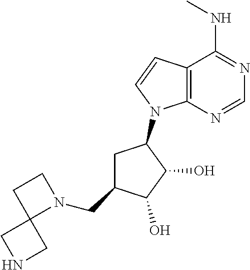 [SMILES: CNc1ncnc2c1ccn2[C@@H]1C[C@H](CN2CCC23CNC3)[C@@H](O)[C@H]1O] | D | + | IV | NA | NA | NA |
| 87 | 446 (318) | 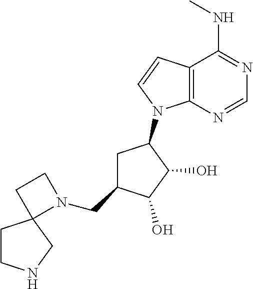 [SMILES: CNc1ncnc2c1ccn2[C@@H]1C[C@H](CN2CCC23CCNC3)[C@@H](O)[C@H]1O] | D | + | IV | NA | NA | NA |
| 88 | 447 (319) | 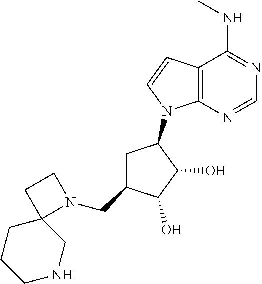 [SMILES: CNc1ncnc2c1ccn2[C@@H]1C[C@H](CN2CCC23CCCNC3)[C@@H](O)[C@H]1O] | D | + | IV | NA | NA | NA |
| 89 | 448 (320) | 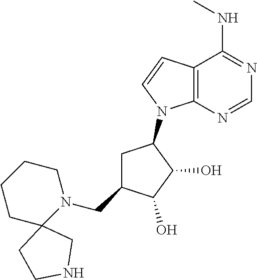 [SMILES: CNc1ncnc2c1ccn2[C@@H]1C[C@H](CN2CCCCC23CCNC3)[C@@H](O)[C@H]1O] | D | + | IV | NA | NA | NA |
| 90 | 449 (321) | 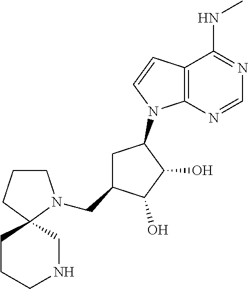 [SMILES: CNc1ncnc2c1ccn2[C@@H]1C[C@H](CN2CCC[C@]23CCCNC3)[C@@H](O)[C@H]1O] | D | + | IV | NA | NA | NA |
| 91 | 450 (322) | 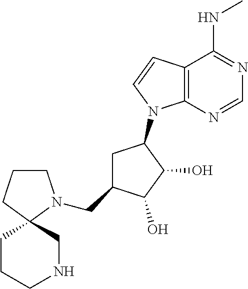 [SMILES: CNc1ncnc2c1ccn2[C@@H]1C[C@H](CN2CCC[C@@]23CCCNC3)[C@@H](O)[C@H]1O] | D | + | IV | NA | NA | NA |
| 92 | 451 (323) | 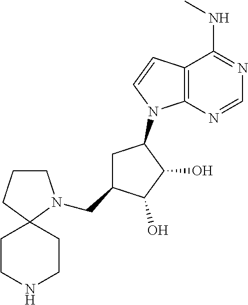 [SMILES: CNc1ncnc2c1ccn2[C@@H]1C[C@H](CN2CCCC23CCNCC3)[C@@H](O)[C@H]1O] | D | + | IV | NA | NA | NA |
| 93 | 452 (324) | 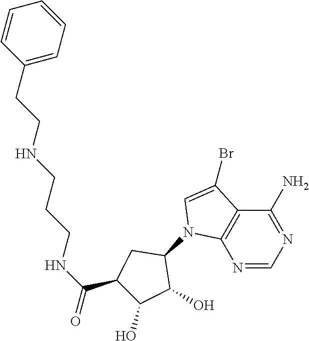 [SMILES: Nc1ncnc2c1c(Br)cn2[C@@H]1C[C@H](C(=O)NCCCNCCc2ccccc2)[C@@H](O)[C@H]1O] | B | +++ | II | * | NA | NA |
| 94 | 453 (325) | 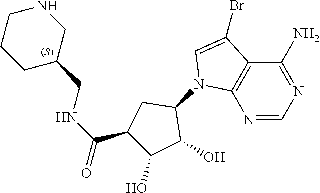 | C | ++ | IV | NA | NA | NA |
| 95 | 454 (326) | 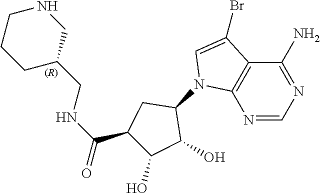 | C | ++ | IV | NA | NA | NA |
| 96 | 455 (327) | 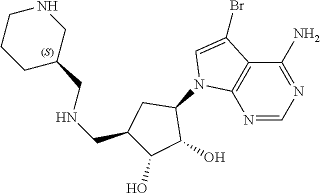 | D | ++ | IV | NA | NA | NA |
| 97 | 456 (328) | 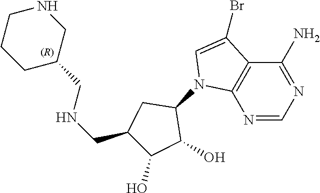 | D | ++ | IV | NA | NA | NA |
| 98 | 457 (329) | 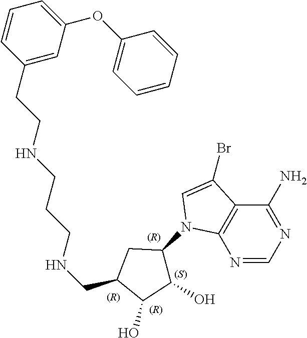 | B | +++ | II | *** | NA | NA |
| 99 | 458 (330) | 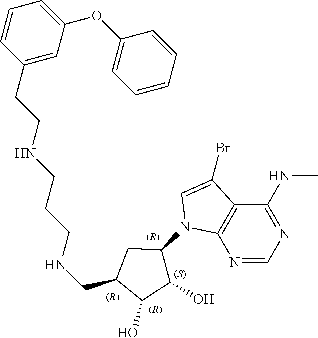 | D | + | IV | NA | NA | NA |
| 100 | 459 (332) | 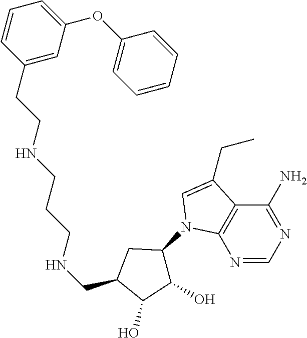 [SMILES: CCc1cn([C@@H]2C[C@H](CNCCCNCCc3cccc(Oc4ccccc4)c3)[C@@H](O)[C@H]2O)c2ncnc(N)c12] | B | +++ | III | ** | NA | NA |
| 101 | 460 (333) | 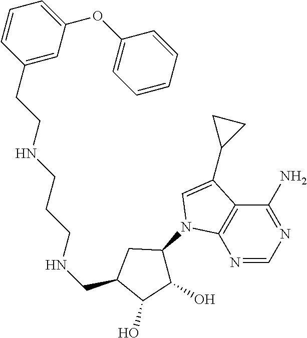 [SMILES: Nc1ncnc2c1c(C1CC1)cn2[C@@H]1C[C@H](CNCCCNCCc2cccc(Oc3ccccc3)c2)[C@@H](O)[C@H]1O] | B | +++ | II | ** | NA | NA |
| 102 | 461 (334) | 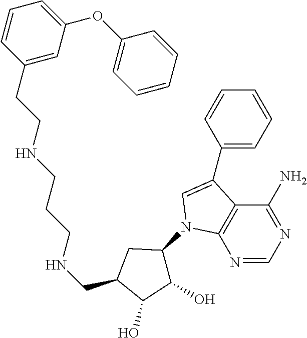 [SMILES: Nc1ncnc2c1c(-c1ccccc1)cn2[C@@H]1C[C@H](CNCCCNCCc2cccc(Oc3ccccc3)c2)[C@@H](O)[C@H]1O] | C | +++ | II | *** | NA | NA |
| 103 | 462 (336) | 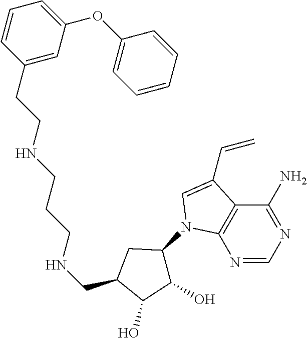 [SMILES: C=Cc1cn([C@@H]2C[C@H](CNCCCNCCc3cccc(Oc4ccccc4)c3)[C@@H](O)[C@H]2O)c2ncnc(N)c12] | B | +++ | III | ** | NA | NA |
| 104 | 463 (337) | 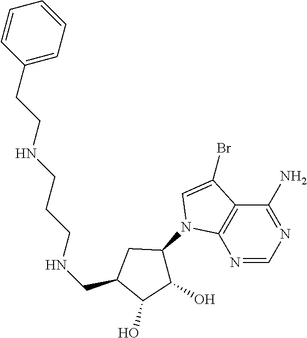 [SMILES: Nc1ncnc2c1c(Br)cn2[C@@H]1C[C@H](CNCCCNCCc2ccccc2)[C@@H](O)[C@H]1O] | B | +++ | IV | * | NA | NA |
| 105 | 464 (338) | 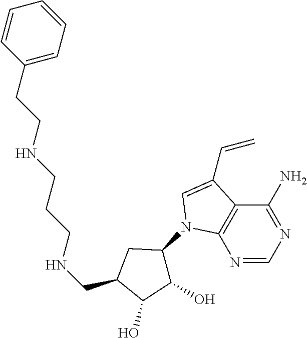 [SMILES: C=Cc1cn([C@@H]2C[C@H](CNCCCNCCc3ccccc3)[C@@H](O)[C@H]2O)c2ncnc(N)c12] | B | ++ | III | * | NA | NA |
| 106 | 465 (339) | 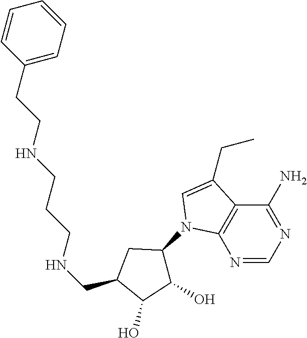 [SMILES: CCc1cn([C@@H]2C[C@H](CNCCCNCCc3ccccc3)[C@@H](O)[C@H]2O)c2ncnc(N)c12] | C | ++ | III | * | NA | NA |
| 107 | 466 (340) | 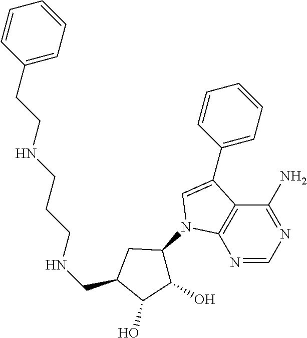 [SMILES: Nc1ncnc2c1c(-c1ccccc1)cn2[C@@H]1C[C@H](CNCCCNCCc2ccccc2)[C@@H](O)[C@H]1O] | C | ++ | II | ** | NA | NA |
| 108 | 467 (341) | 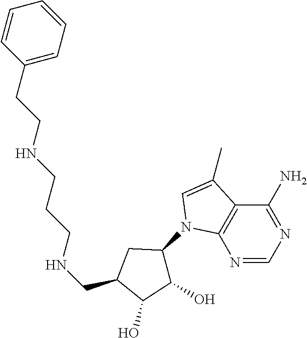 [SMILES: Cc1cn([C@@H]2C[C@H](CNCCCNCCc3ccccc3)[C@@H](O)[C@H]2O)c2ncnc(N)c12] | C | ++ | III | NA | NA | NA |
| 109 | 468 (342) | 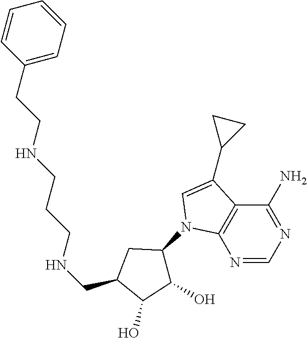 [SMILES: Nc1ncnc2c1c(C1CC1)cn2[C@@H]1C[C@H](CNCCCNCCc2ccccc2)[C@@H](O)[C@H]1O] | C | +++ | III | * | NA | NA |
| 110 | 469 (343) | 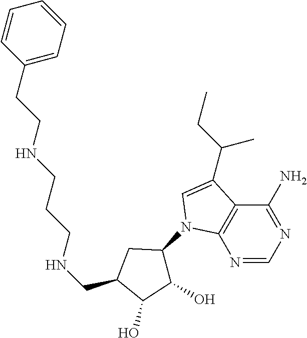 [SMILES: CCC(C)c1cn([C@@H]2C[C@H](CNCCCNCCc3ccccc3)[C@@H](O)[C@H]2O)c2ncnc(N)c12] | C | ++ | III | * | NA | NA |
| 111 | 470 (344) | 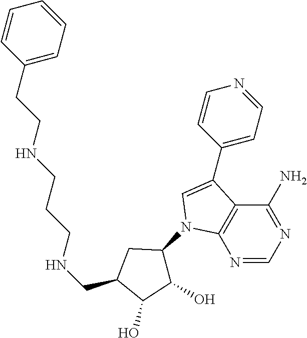 [SMILES: Nc1ncnc2c1c(-c1ccncc1)cn2[C@@H]1C[C@H](CNCCCNCCc2ccccc2)[C@@H](O)[C@H]1O] | C | ++ | II | NA | NA | NA |
| 112 | 471 (345) | 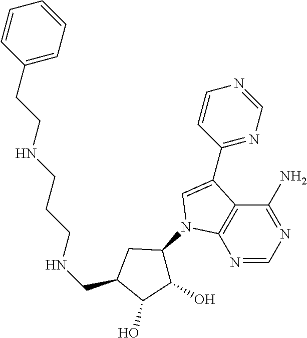 [SMILES: Nc1ncnc2c1c(-c1ccncn1)cn2[C@@H]1C[C@H](CNCCCNCCc2ccccc2)[C@@H](O)[C@H]1O] | C | +++ | II | NA | NA | NA |
| 113 | 472 (346) | 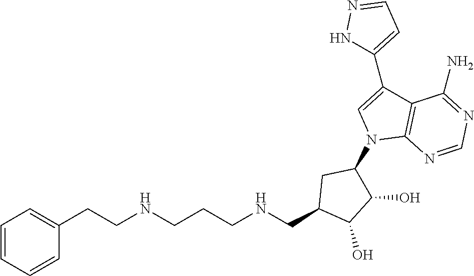 [SMILES: Nc1ncnc2c1c(-c1ccn[nH]1)cn2[C@@H]1C[C@H](CNCCCNCCc2ccccc2)[C@@H](O)[C@H]1O] | B | +++ | II | NA | NA | NA |
| 114 | 473 (347) | 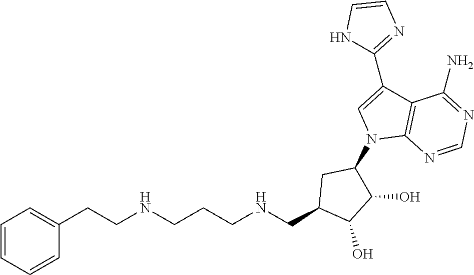 [SMILES: Nc1ncnc2c1c(-c1ncc[nH]1)cn2[C@@H]1C[C@H](CNCCCNCCc2ccccc2)[C@@H](O)[C@H]1O] | B | +++ | II | NA | NA | NA |
| 115 | 474 (348) | 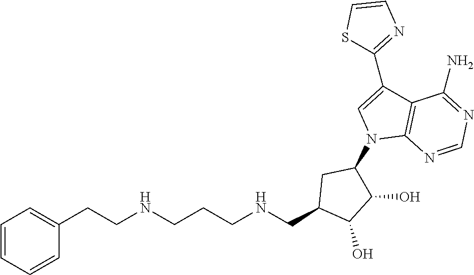 [SMILES: Nc1ncnc2c1c(-c1nccs1)cn2[C@@H]1C[C@H](CNCCCNCCc2ccccc2)[C@@H](O)[C@H]1O] | B | +++ | III | ** | NA | NA |
| 116 | 475 (349) | 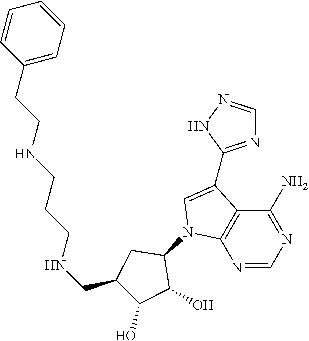 [SMILES: Nc1ncnc2c1c(-c1ncn[nH]1)cn2[C@@H]1C[C@H](CNCCCNCCc2ccccc2)[C@@H](O)[C@H]1O] | B | +++ | II | NA | NA | NA |
| 117 | 476 (350) | 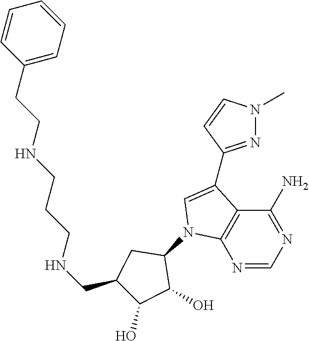 [SMILES: Cn1ccc(-c2cn([C@@H]3C[C@H](CNCCCNCCc4ccccc4)[C@@H](O)[C@H]3O)c3ncnc(N)c23)n1] | C | +++ | II | * | NA | NA |
| 118 | 477 (351) | 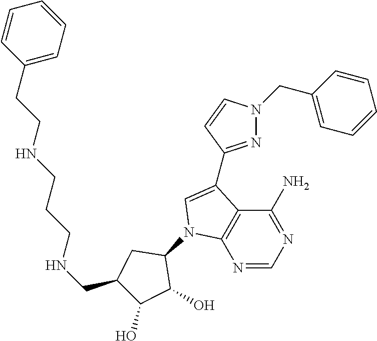 [SMILES: Nc1ncnc2c1c(-c1ccn(Cc3ccccc3)n1)cn2[C@@H]1C[C@H](CNCCCNCCc2ccccc2)[C@@H](O)[C@H]1O] | B | +++ | III | * | NA | NA |
| 119 | 478 (352) | 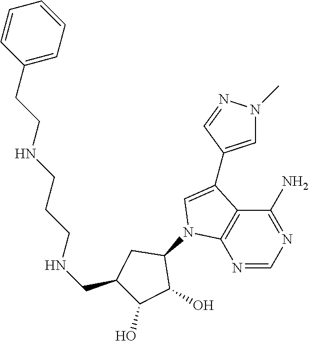 [SMILES: Cn1cc(-c2cn([C@@H]3C[C@H](CNCCCNCCc4ccccc4)[C@@H](O)[C@H]3O)c3ncnc(N)c23)cn1] | B | +++ | III | NA | NA | NA |
| 120 | 479 (353) | 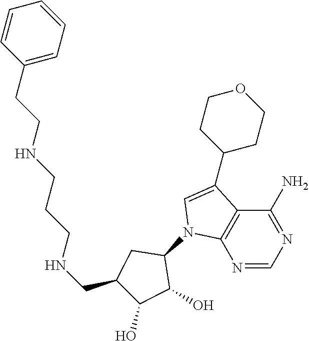 [SMILES: Nc1ncnc2c1c(C1CCOCC1)cn2[C@@H]1C[C@H](CNCCCNCCc2ccccc2)[C@@H](O)[C@H]1O] | C | ++ | III | NA | NA | NA |
| 121 | 480 (354) | 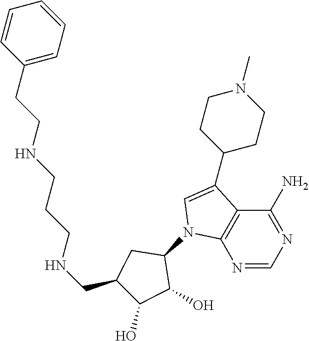 [SMILES: CN1CCC(c2cn([C@@H]3C[C@H](CNCCCNCCc4ccccc4)[C@@H](O)[C@H]3O)c3ncnc(N)c23)CC1] | D | + | III | NA | NA | NA |
| 122 | 481 (355) | 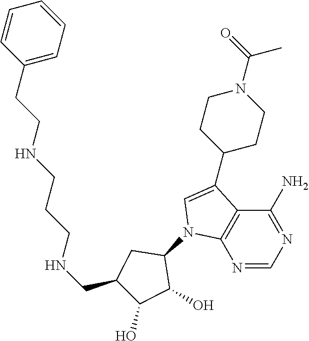 [SMILES: CC(=O)N1CCC(c2cn([C@@H]3C[C@H](CNCCCNCCc4ccccc4)[C@@H](O)[C@H]3O)c3ncnc(N)c23)CC1] | C | ++ | III | NA | NA | NA |
| 123 | 482 (356) | 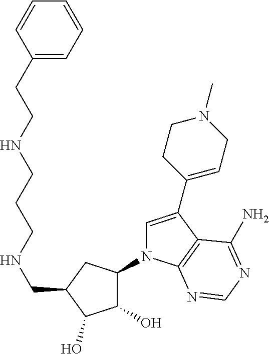 [SMILES: CN1CC=C(c2cn([C@@H]3C[C@H](CNCCCNCCc4ccccc4)[C@@H](O)[C@H]3O)c3ncnc(N)c23)CC1] | D | ++ | III | NA | NA | NA |
| 124 | 483 (357) | 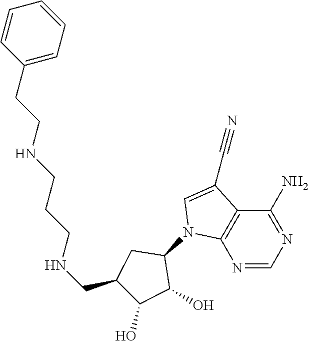 [SMILES: N#Cc1cn([C@@H]2C[C@H](CNCCCNCCc3ccccc3)[C@@H](O)[C@H]2O)c2ncnc(N)c12] | C | ++ | III | NA | NA | NA |
| 125 | 484 (358) | 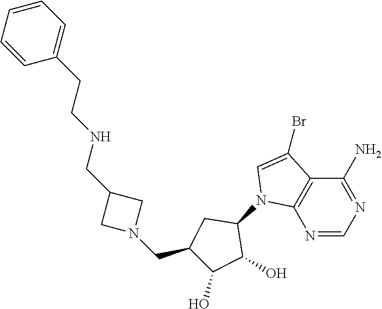 [SMILES: Nc1ncnc2c1c(Br)cn2[C@@H]1C[C@H](CN2CC(CNCCc3ccccc3)C2)[C@@H](O)[C@H]1O] | B | +++ | II | * | NA | NA |
| 126 | 485 (359) | 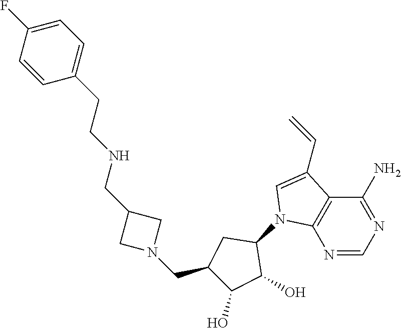 [SMILES: C=Cc1cn([C@@H]2C[C@H](CN3CC(CNCCc4ccc(F)cc4)C3)[C@@H](O)[C@H]2O)c2ncnc(N)c12] | B | +++ | III | * | NA | NA |
| 127 | 487 (360) | 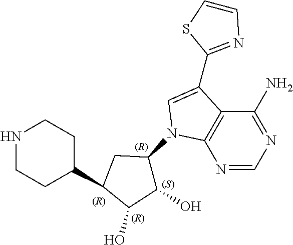 | B | +++ | III | * | NA | NA |
| 128 | 683 | 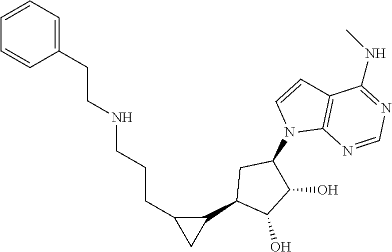 [SMILES: CNc1ncnc2c1ccn2[C@@H]1C[C@H](C2CC2CCCNCCc2ccccc2)[C@@H](O)[C@H]1O] | D | + | | NA | NA | NA |
| 129 | 602 | 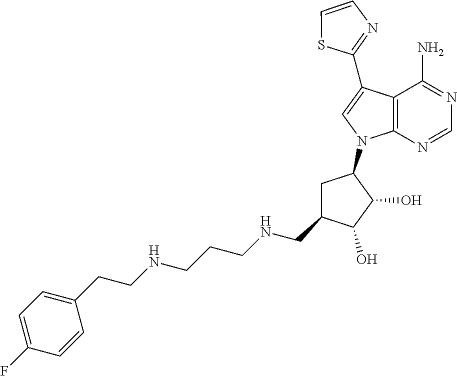 [SMILES: Nc1ncnc2c1c(-c1nccs1)cn2[C@@H]1C[C@H](CNCCCNCCc2ccc(F)cc2)[C@@H](O)[C@H]1O] | B | +++ | II | ** | NA | NA |
| 130 | 600 | 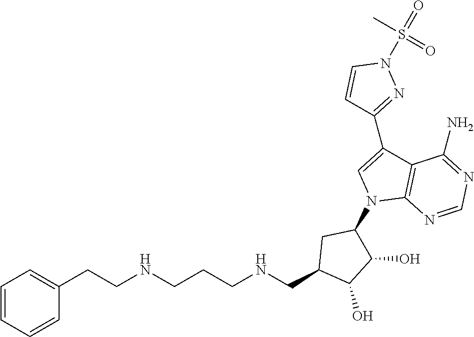 [SMILES: CS(=O)(=O)n1ccc(-c2cn([C@@H]3C[C@H](CNCCCNCCc4ccccc4)[C@@H](O)[C@H]3O)c3ncnc(N)c23)n1] | A | +++ | I | * | NA | NA |
| 131 | 603 | 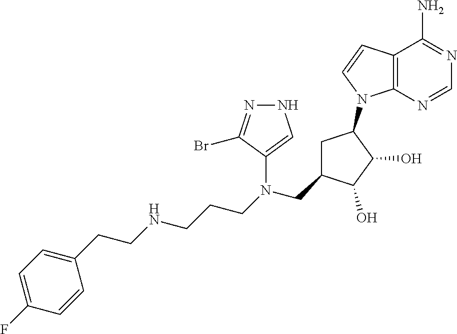 [SMILES: Nc1ncnc2c1ccn2[C@@H]1C[C@H](CN(CCCNCCc2ccc(F)cc2)c2c[nH]nc2Br)[C@@H](O)[C@H]1O] | A | +++ | II | * | NA | NA |
| 132 | 601 | 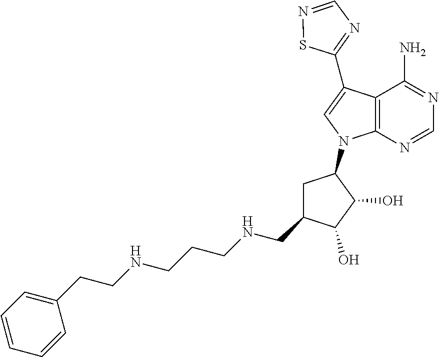 [SMILES: Nc1ncnc2c1c(-c1ncns1)cn2[C@@H]1C[C@H](CNCCCNCCc2ccccc2)[C@@H](O)[C@H]1O] | C | ++ | III | * | NA | NA |
| 133 | 624 | 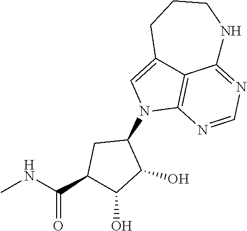 [SMILES: CNC(=O)[C@H]1C[C@@H](n2cc3c4c(ncnc42)NCCC3)[C@H](O)[C@@H]1O] | D | + | IV | NA | NA | NA |
| 134 | 599 | 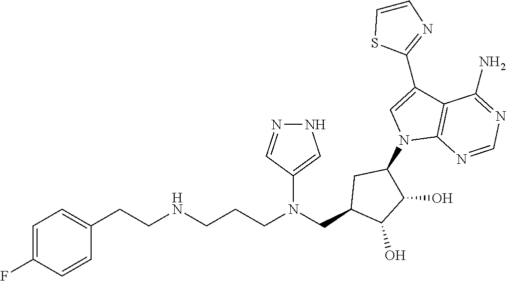 [SMILES: Nc1ncnc2c1c(-c1nccs1)cn2[C@@H]1C[C@H](CN(CCCNCCc2ccc(F)cc2)c2cn[nH]c2)[C@@H](O)[C@H]1O] | A | +++ | I | * | * | ## |
| 135 | 626 | 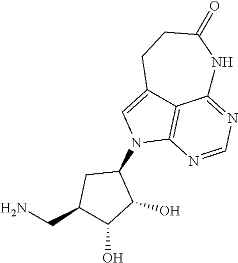 [SMILES: NC[C@H]1C[C@@H](n2cc3c4c(ncnc42)NC(=O)CC3)[C@H](O)[C@@H]1O] | D | + | IV | NA | NA | NA |
| 136 | 625 | 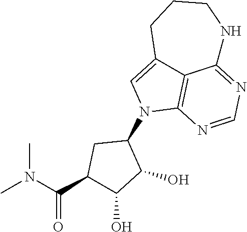 [SMILES: CN(C)C(=O)[C@H]1C[C@@H](n2cc3c4c(ncnc42)NCCC3)[C@H](O)[C@@H]1O] | D | + | IV | NA | NA | NA |
| 137 | 629 | 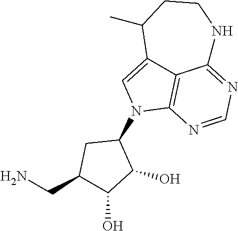 [SMILES: CC1CCNc2ncnc3c2c1cn3[C@@H]1C[C@H](CN)[C@@H](O)[C@H]1O] | D | + | IV | NA | NA | NA |
| 138 | 486 | 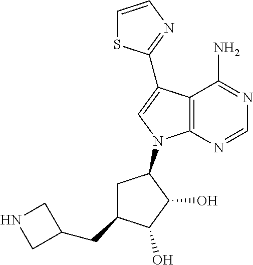 [SMILES: Nc1ncnc2c1c(-c1nccs1)cn2[C@@H]1C[C@H](CC2CNC2)[C@@H](O)[C@H]1O] | D | + | IV | NA | NA | NA |
| 139 | 606 | 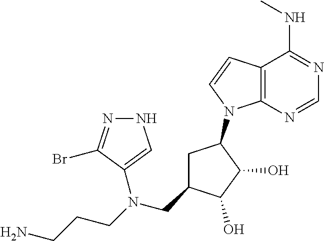 [SMILES: CNc1ncnc2c1ccn2[C@@H]1C[C@H](CN(CCCN)c2c[nH]nc2Br)[C@@H](O)[C@H]1O] | D | + | IV | NA | NA | NA |
| 140 | 604 | 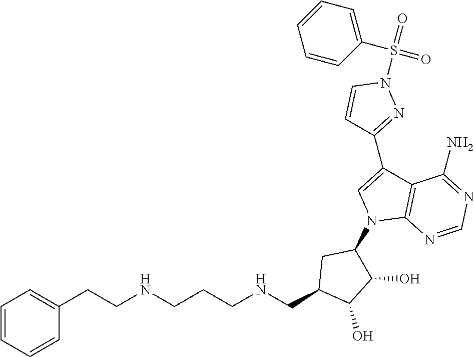 [SMILES: Nc1ncnc2c1c(-c1ccn(S(=O)(=O)c3ccccc3)n1)cn2[C@@H]1C[C@H](CNCCCNCCc2ccccc2)[C@@H](O)[C@H]1O] | A | +++ | I | NA | ** | NA |
| 141 | 627 | 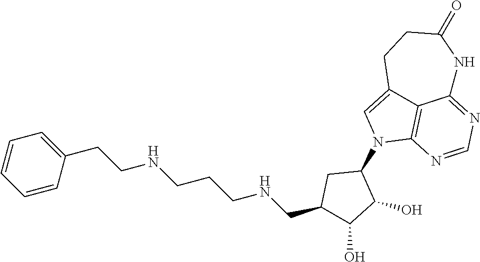 [SMILES: O=C1CCc2cn([C@@H]3C[C@H](CNCCCNCCc4ccccc4)[C@@H](O)[C@H]3O)c3ncnc(c23)N1] | C | + | IV | NA | * | NA |
| 142 | 605 | 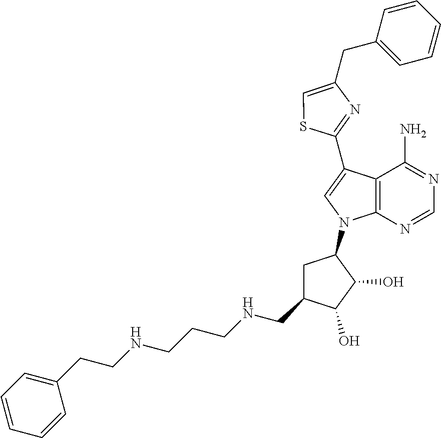 [SMILES: Nc1ncnc2c1c(-c1nc(Cc3ccccc3)cs1)cn2[C@@H]1C[C@H](CNCCCNCCc2ccccc2)[C@@H](O)[C@H]1O] | B | +++ | II | *** | *** | ## |
| 143 | 609 | 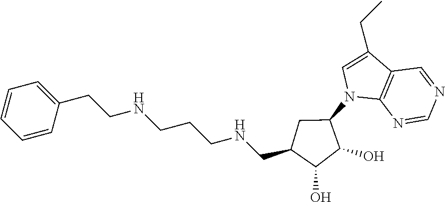 [SMILES: CCc1cn([C@@H]2C[C@H](CNCCCNCCc3ccccc3)[C@@H](O)[C@H]2O)c2ncncc12] | C | + | IV | NA | NA | NA |
| 144 | 611 | 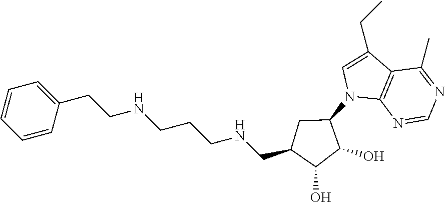 [SMILES: CCc1cn([C@@H]2C[C@H](CNCCCNCCc3ccccc3)[C@@H](O)[C@H]2O)c2ncnc(C)c12] | D | + | IV | NA | NA | NA |
| 145 | 608 | 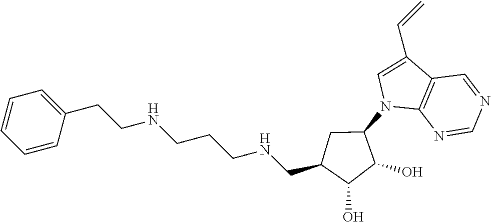 [SMILES: C=Cc1cn([C@@H]2C[C@H](CNCCCNCCc3ccccc3)[C@@H](O)[C@H]2O)c2ncncc12] | C | + | IV | NA | ** | NA |
| 146 | 610 | 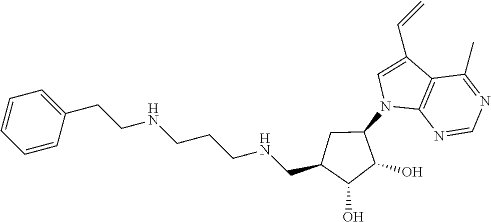 [SMILES: C=Cc1cn([C@@H]2C[C@H](CNCCCNCCc3ccccc3)[C@@H](O)[C@H]2O)c2ncnc(C)c12] | D | + | IV | NA | NA | NA |
| 147 | 630 | 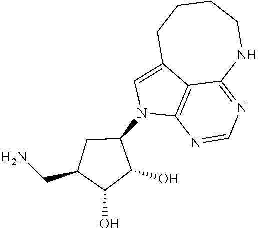 [SMILES: NC[C@H]1C[C@@H](n2cc3c4c(ncnc42)NCCCC3)[C@H](O)[C@@H]1O] | D | + | IV | NA | NA | NA |
| 148 | 489 | 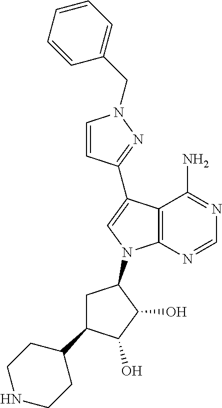 [SMILES: Nc1ncnc2c1c(-c1ccn(Cc3ccccc3)n1)cn2[C@@H]1C[C@H](C2CCNCC2)[C@@H](O)[C@H]1O] | B | +++ | III | NA | * | NA |
| 149 | 633 | 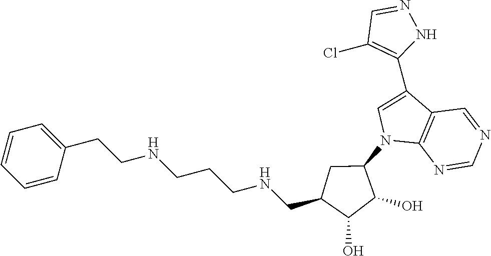 [SMILES: O[C@@H]1[C@@H](CNCCCNCCc2ccccc2)C[C@@H](n2cc(-c3[nH]ncc3Cl)c3cncnc32)[C@@H]1O] | D | + | III | NA | NA | NA |
| 150 | 632 | 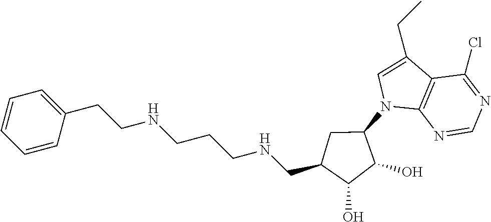 [SMILES: CCc1cn([C@@H]2C[C@H](CNCCCNCCc3ccccc3)[C@@H](O)[C@H]2O)c2ncnc(Cl)c12] | D | + | IV | NA | NA | NA |
| 151 | 612 | 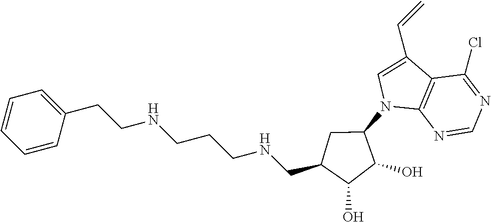 [SMILES: C=Cc1cn([C@@H]2C[C@H](CNCCCNCCc3ccccc3)[C@@H](O)[C@H]2O)c2ncnc(Cl)c12] | D | + | IV | NA | NA | NA |
| 152 | 488 | 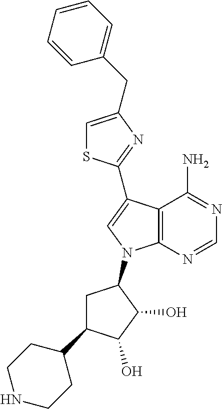 [SMILES: Nc1ncnc2c1c(-c1nc(Cc3ccccc3)cs1)cn2[C@@H]1C[C@H](C2CCNCC2)[C@@H](O)[C@H]1O] | B | ++ | III | NA | ** | NA |
| 153 | 614 | 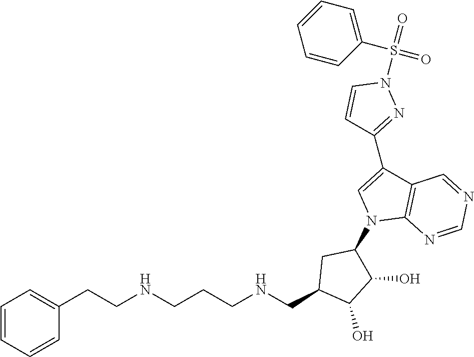 [SMILES: O=S(=O)(c1ccccc1)n1ccc(-c2cn([C@@H]3C[C@H](CNCCCNCCc4ccccc4)[C@@H](O)[C@H]3O)c3ncncc23)n1] | C | ++ | III | NA | ** | NA |
| 154 | 490 | 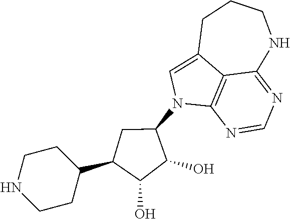 [SMILES: O[C@@H]1[C@H](O)[C@@H](C2CCNCC2)C[C@H]1n1cc2c3c(ncnc31)NCCC2] | C | ++ | III | NA | * | NA |
| 155 | 379 | 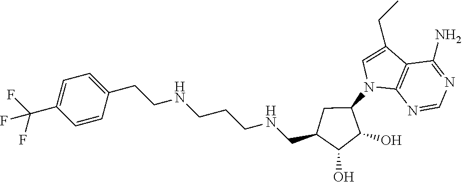 [SMILES: CCc1cn([C@@H]2C[C@H](CNCCCNCCc3ccc(C(F)(F)F)cc3)[C@@H](O)[C@H]2O)c2ncnc(N)c12] | C | ++ | III | NA | ** | NA |
| 156 | 613 | 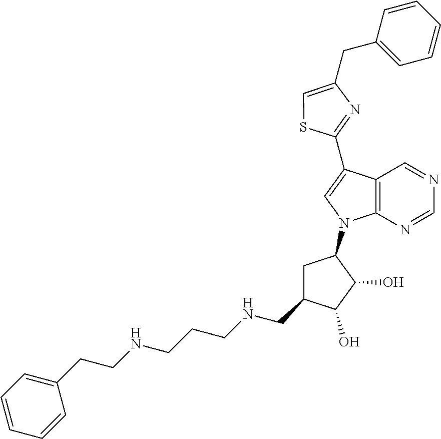 [SMILES: O[C@@H]1[C@@H](CNCCCNCCc2ccccc2)C[C@@H](n2cc(-c3nc(Cc4ccccc4)cs3)c3cncnc32)[C@@H]1O] | D | ++ | III | NA | *** | NA |
| 157 | 631 | 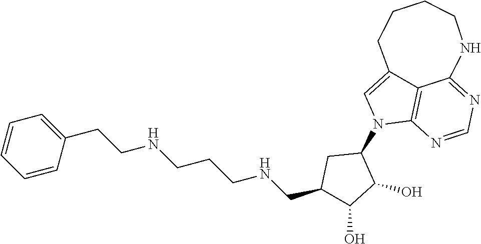 [SMILES: O[C@@H]1[C@@H](CNCCCNCCc2ccccc2)C[C@@H](n2cc3c4c(ncnc42)NCCCC3)[C@@H]1O] | B | ++ | III | NA | * | NA |
| 158 | 607 | 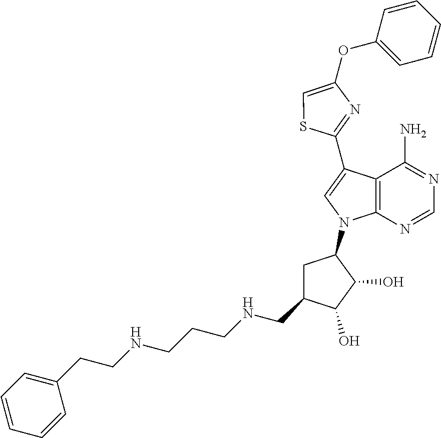 [SMILES: Nc1ncnc2c1c(-c1nc(Oc3ccccc3)cs1)cn2[C@@H]1C[C@H](CNCCCNCCc2ccccc2)[C@@H](O)[C@H]1O] | B | +++ | II | NA | *** | NA |
| 159 | 628 | 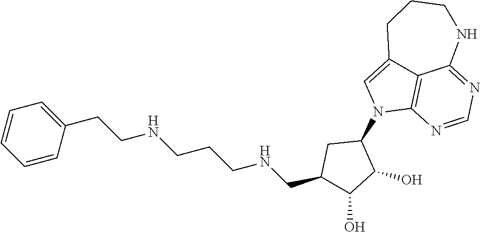 [SMILES: O[C@@H]1[C@@H](CNCCCNCCc2ccccc2)C[C@@H](n2cc3c4c(ncnc42)NCCC3)[C@@H]1O] | C | ++ | III | NA | * | NA |
| 160 | 385 | 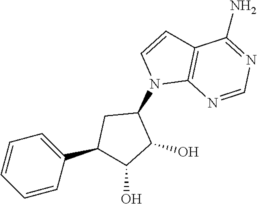 [SMILES: Nc1ncnc2c1ccn2[C@@H]1C[C@H](c2ccccc2)[C@@H](O)[C@H]1O] | D | + | IV | NA | NA | NA |
| 161 | 389 | 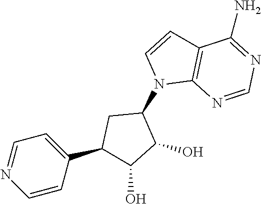 [SMILES: Nc1ncnc2c1ccn2[C@@H]1C[C@H](c2ccncc2)[C@@H](O)[C@H]1O] | D | + | IV | NA | NA | NA |
| 162 | 384 | 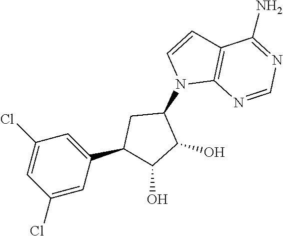 [SMILES: Nc1ncnc2c1ccn2[C@@H]1C[C@H](c2cc(Cl)cc(Cl)c2)[C@@H](O)[C@H]1O] | D | + | IV | NA | NA | NA |
| 163 | 381 | 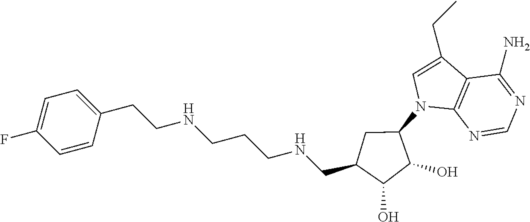 [SMILES: CCc1cn([C@@H]2C[C@H](CNCCCNCCc3ccc(F)cc3)[C@@H](O)[C@H]2O)c2ncnc(N)c12] | C | ++ | III | NA | NA | NA |
| 164 | 637 | 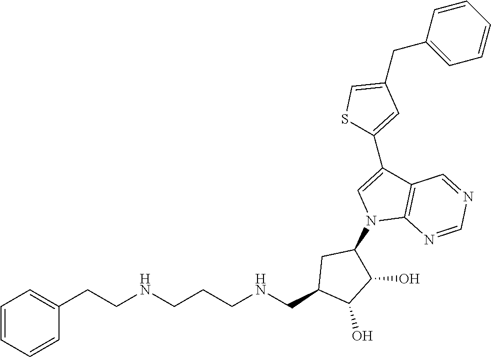 [SMILES: O[C@@H]1[C@@H](CNCCCNCCc2ccccc2)C[C@@H](n2cc(-c3cc(Cc4ccccc4)cs3)c3cncnc32)[C@@H]1O] | D | ++ | III | NA | *** | NA |
| 165 | 522 | 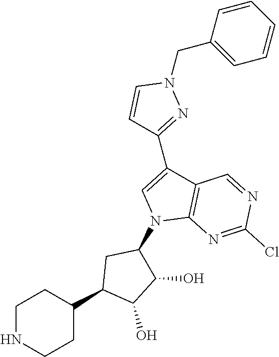 [SMILES: O[C@@H]1[C@H](O)[C@@H](C2CCNCC2)C[C@H]1n1cc(-c2ccn(Cc3ccccc3)n2)c2cnc(Cl)nc21] | C | +++ | III | NA | * | NA |
| 166 | 383 | 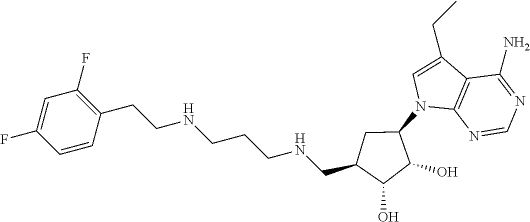 [SMILES: CCc1cn([C@@H]2C[C@H](CNCCCNCCc3ccc(F)cc3F)[C@@H](O)[C@H]2O)c2ncnc(N)c12] | B | ++ | III | NA | ** | NA |
| 167 | 501 | 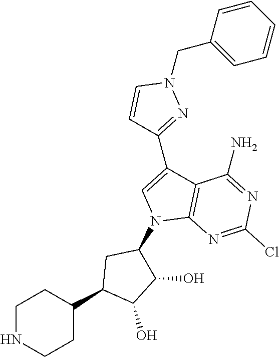 [SMILES: Nc1nc(Cl)nc2c1c(-c1ccn(Cc3ccccc3)n1)cn2[C@@H]1C[C@H](C2CCNCC2)[C@@H](O)[C@H]1O] | A | ++++ | I | NA | * | NA |
| 168 | 638 | 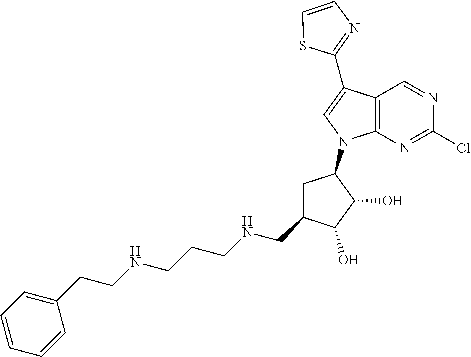 [SMILES: O[C@@H]1[C@@H](CNCCCNCCc2ccccc2)C[C@@H](n2cc(-c3nccs3)c3cnc(Cl)nc32)[C@@H]1O] | C | +++ | III | NA | ** | NA |
| 169 | 634 | 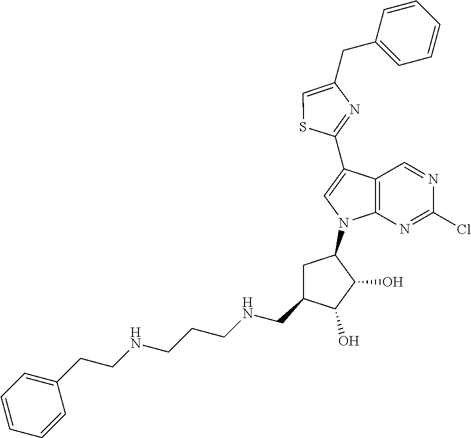 [SMILES: O[C@@H]1[C@@H](CNCCCNCCc2ccccc2)C[C@@H](n2cc(-c3nc(Cc4ccccc4)cs3)c3cnc(Cl)nc32)[C@@H]1O] | B | +++ | III | NA | *** | ## |
| 170 | 636 | 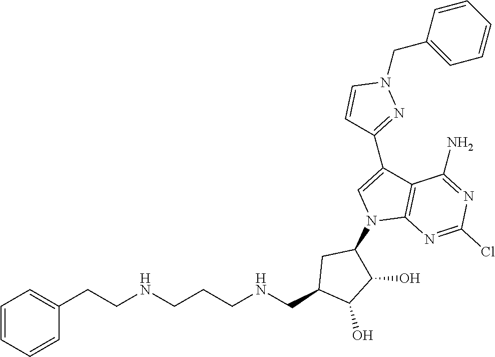 [SMILES: Nc1nc(Cl)nc2c1c(-c1ccn(Cc3ccccc3)n1)cn2[C@@H]1C[C@H](CNCCCNCCc2ccccc2)[C@@H](O)[C@H]1O] | A | ++++ | II | NA | *** | ## |
| 171 | 503 | 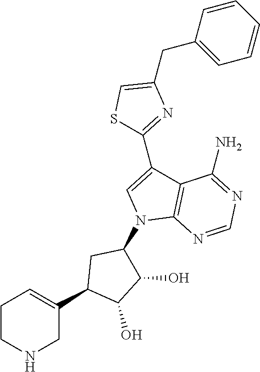 [SMILES: Nc1ncnc2c1c(-c1nc(Cc3ccccc3)cs1)cn2[C@@H]1C[C@H](C2=CCCNC2)[C@@H](O)[C@H]1O] | C | +++ | III | NA | *** | NA |
| 172 | 502 | 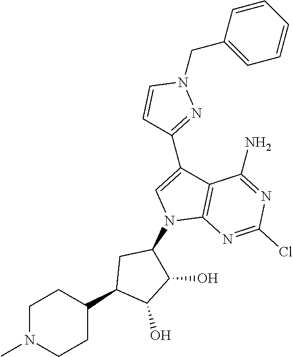 [SMILES: CN1CCC([C@H]2C[C@@H](n3cc(-c4ccn(Cc5ccccc5)n4)c4c(N)nc(Cl)nc43)[C@H](O)[C@@H]2O)CC1] | A | ++++ | II | NA | *** | NA |
| 173 | 394 | 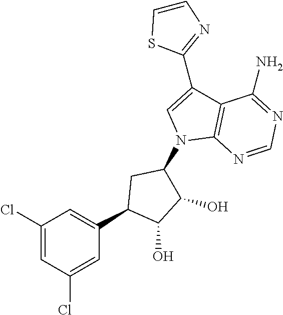 [SMILES: Nc1ncnc2c1c(-c1nccs1)cn2[C@@H]1C[C@H](c2cc(Cl)cc(Cl)c2)[C@@H](O)[C@H]1O] | D | + | IV | NA | | NA |
| 174 | 617 | 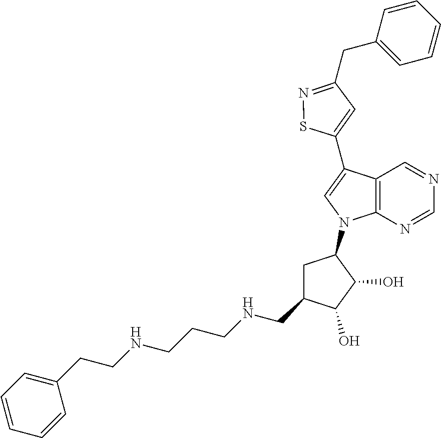 [SMILES: O[C@@H]1[C@@H](CNCCCNCCc2ccccc2)C[C@@H](n2cc(-c3cc(Cc4ccccc4)ns3)c3cncnc32)[C@@H]1O] | D | ++ | III | NA | *** | NA |
| 175 | 593R | 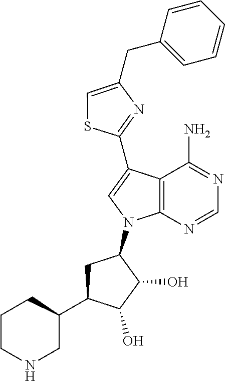 [SMILES: Nc1ncnc2c1c(-c1nc(Cc3ccccc3)cs1)cn2[C@@H]1C[C@H]([C@@H]2CCCNC2)[C@@H](O)[C@H]1O] | C | +++ | III | NA | ** | NA |
| 176 | 593S | 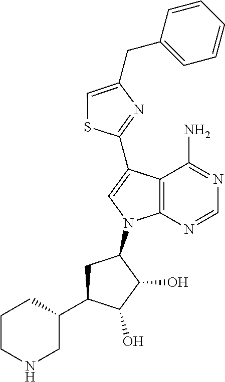 [SMILES: Nc1ncnc2c1c(-c1nc(Cc3ccccc3)cs1)cn2[C@@H]1C[C@H]([C@H]2CCCNC2)[C@@H](O)[C@H]1O] | C | ++ | III | NA | ** | NA |
| 177 | 387 | 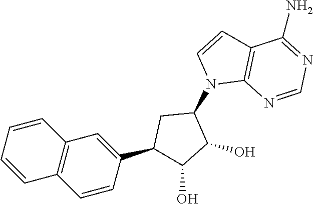 [SMILES: Nc1ncnc2c1ccn2[C@@H]1C[C@H](c2ccc3ccccc3c2)[C@@H](O)[C@H]1O] | D | + | IV | NA | NA | NA |
| 178 | 395 | 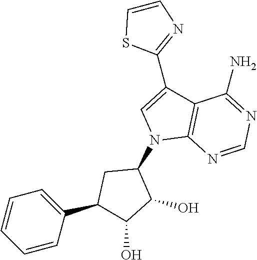 [SMILES: Nc1ncnc2c1c(-c1nccs1)cn2[C@@H]1C[C@H](c2ccccc2)[C@@H](O)[C@H]1O] | D | + | IV | NA | NA | NA |
| 179 | 390 | 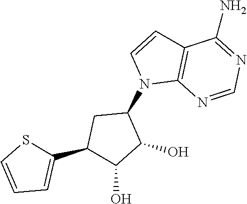 [SMILES: Nc1ncnc2c1ccn2[C@@H]1C[C@H](c2cccs2)[C@@H](O)[C@H]1O] | D | + | IV | NA | NA | NA |
| 180 | 616 | 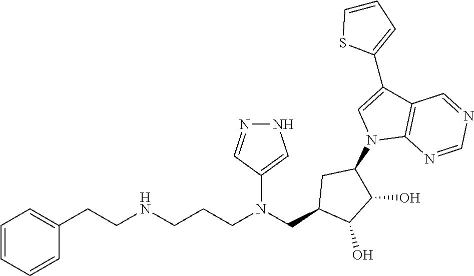 [SMILES: O[C@@H]1[C@@H](CN(CCCNCCc2ccccc2)c2cn[nH]c2)C[C@@H](n2cc(-c3cccs3)c3cncnc32)[C@@H]1O] | B | ++ | III | NA | * | NA |
| 181 | 639 | 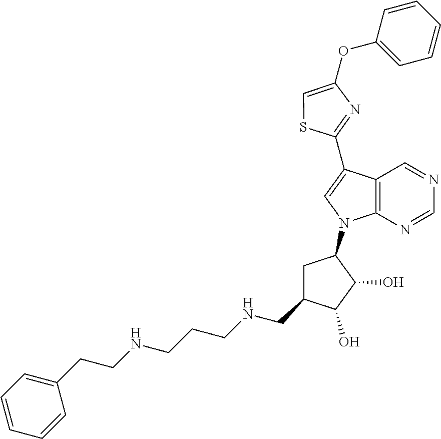 [SMILES: O[C@@H]1[C@@H](CNCCCNCCc2ccccc2)C[C@@H](n2cc(-c3nc(Oc4ccccc4)cs3)c3cncnc32)[C@@H]1O] | D | ++ | III | NA | | ### |
| 182 | 621 | 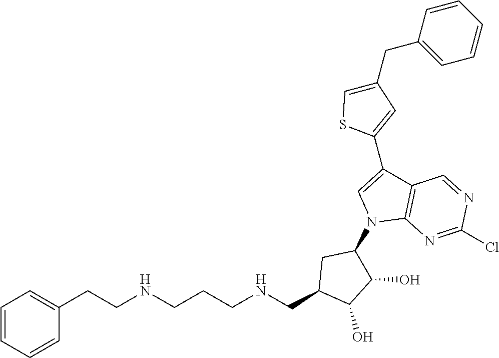 [SMILES: O[C@@H]1[C@@H](CNCCCNCCc2ccccc2)C[C@@H](n2cc(-c3cc(Cc4ccccc4)cs3)c3cnc(Cl)nc32)[C@@H]1O] | C | +++ | III | NA | *** | ### |
| 183 | 635 | 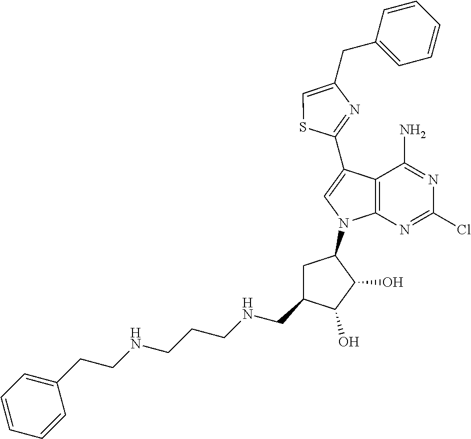 [SMILES: Nc1nc(Cl)nc2c1c(-c1nc(Cc3ccccc3)cs1)cn2[C@@H]1C[C@H](CNCCCNCCc2ccccc2)[C@@H](O)[C@H]1O] | C | +++ | II | NA | *** | ## |
| 184 | 640 | 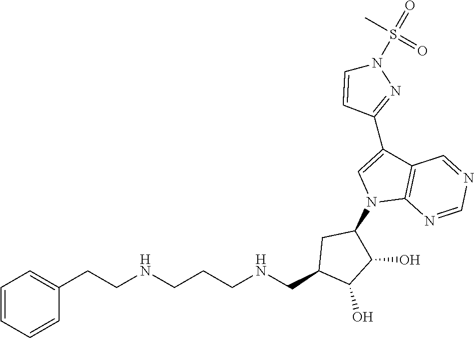 [SMILES: CS(=O)(=O)n1ccc(-c2cn([C@@H]3C[C@H](CNCCCNCCc4ccccc4)[C@@H](O)[C@H]3O)c3ncncc23)n1] | D | ++ | III | NA | * | NA |
| 185 | 589S | 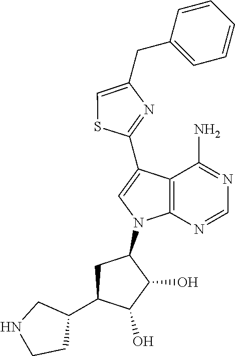 [SMILES: Nc1ncnc2c1c(-c1nc(Cc3ccccc3)cs1)cn2[C@@H]1C[C@H]([C@@H]2CCNC2)[C@@H](O)[C@H]1O] | C | ++ | III | NA | NA | NA |
| 186 | 589R | 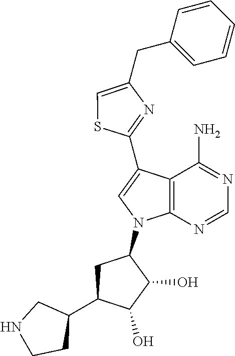 [SMILES: Nc1ncnc2c1c(-c1nc(Cc3ccccc3)cs1)cn2[C@@H]1C[C@H]([C@H]2CCNC2)[C@@H](O)[C@H]1O] | C | ++ | III | NA | NA | NA |
| 187 | 386 | 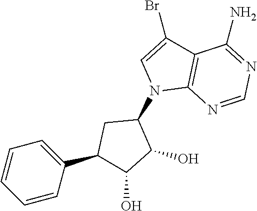 [SMILES: Nc1ncnc2c1c(Br)cn2[C@@H]1C[C@H](c2ccccc2)[C@@H](O)[C@H]1O] | D | + | IV | NA | NA | NA |
| 188 | 396 | 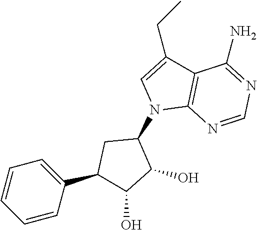 [SMILES: CCc1cn([C@@H]2C[C@H](c3ccccc3)[C@@H](O)[C@H]2O)c2ncnc(N)c12] | D | + | IV | NA | NA | NA |
| 189 | 615 | 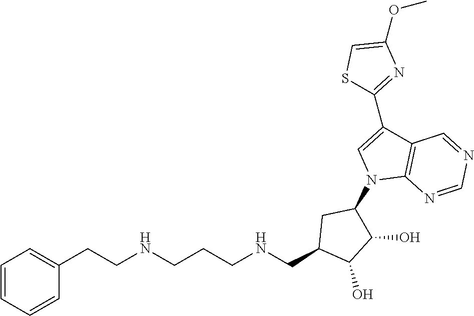 [SMILES: COc1csc(-c2cn([C@@H]3C[C@H](CNCCCNCCc4ccccc4)[C@@H](O)[C@H]3O)c3ncncc23)n1] | D | + | IV | NA | NA | NA |
| 190 | 618 | 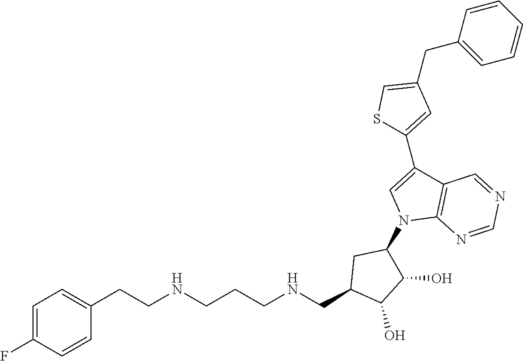 [SMILES: O[C@@H]1[C@@H](CNCCCNCCc2ccc(F)cc2)C[C@@H](n2cc(-c3cc(Cc4ccccc4)cs3)c3cncnc32)[C@@H]1O] | C | ++ | III | NA | *** | NA |
| 191 | 619 | 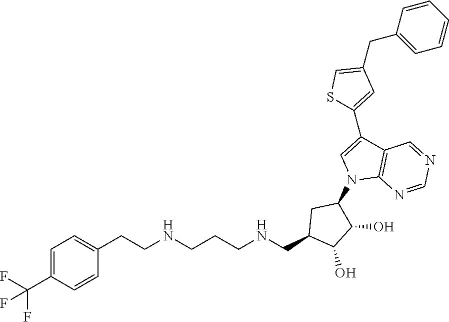 [SMILES: O[C@@H]1[C@@H](CNCCCNCCc2ccc(C(F)(F)F)cc2)C[C@@H](n2cc(-c3cc(Cc4ccccc4)cs3)c3cncnc32)[C@@H]1O] | D | + | IV | NA | | NA |
| 192 | 620 | 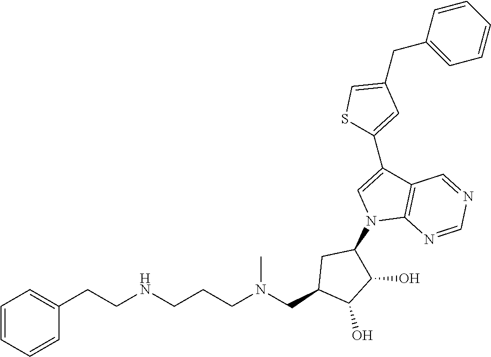 [SMILES: CN(CCCNCCc1ccccc1)C[C@H]1C[C@@H](n2cc(-c3cc(Cc4ccccc4)cs3)c3cncnc32)[C@H](O)[C@@H]1O] | C | ++ | III | NA | *** | ### |
| 193 | 622 | 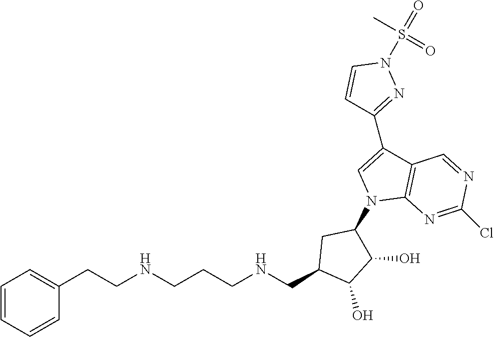 [SMILES: CS(=O)(=O)n1ccc(-c2cn([C@@H]3C[C@H](CNCCCNCCc4ccccc4)[C@@H](O)[C@H]3O)c3nc(Cl)ncc23)n1] | C | +++ | II | NA | ** | NA |
| 194 | 491 | 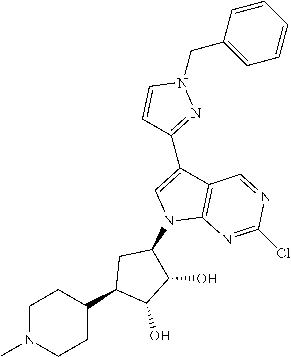 [SMILES: CN1CCC([C@H]2C[C@@H](n3cc(-c4ccn(Cc5ccccc5)n4)c4cnc(Cl)nc43)[C@H](O)[C@@H]2O)CC1] | C | +++ | II | NA | ** | # |
| 195 | 649 | 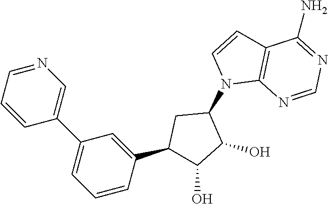 [SMILES: Nc1ncnc2c1ccn2[C@@H]1C[C@H](c2cccc(-c3cccnc3)c2)[C@@H](O)[C@H]1O] | D | + | IV | NA | NA | NA |
| 196 | 515 | 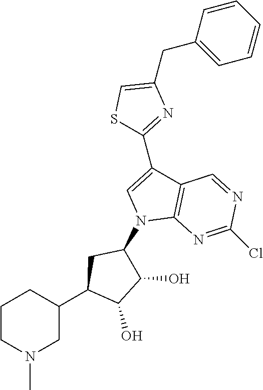 [SMILES: CN1CCCC([C@H]2C[C@@H](n3cc(-c4nc(Cc5ccccc5)cs4)c4cnc(Cl)nc43)[C@H](O)[C@@H]2O)C1] | C | +++ | III | NA | NA | NA |
| 197 | 516 | 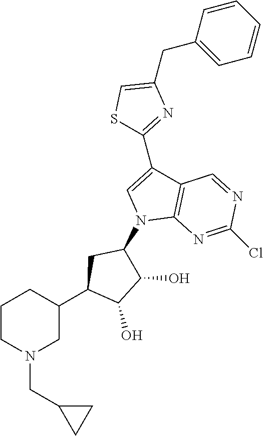 [SMILES: O[C@@H]1[C@H](O)[C@@H](C2CCCN(CC3CC3)C2)C[C@H]1n1cc(-c2nc(Cc3ccccc3)cs2)c2cnc(Cl)nc21] | C | | III | NA | NA | NA |
| 198 | 517 | 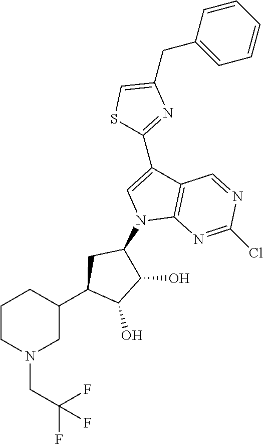 [SMILES: O[C@@H]1[C@H](O)[C@@H](C2CCCN(CC(F)(F)F)C2)C[C@H]1n1cc(-c2nc(Cc3ccccc3)cs2)c2cnc(Cl)nc21] | D | + | IV | NA | NA | NA |
| 199 | 518 | 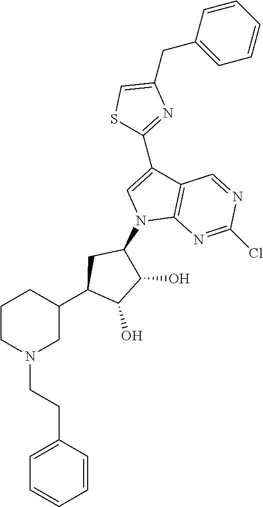 [SMILES: O[C@@H]1[C@H](O)[C@@H](C2CCCN(CCc3ccccc3)C2)C[C@H]1n1cc(-c2nc(Cc3ccccc3)cs2)c2cnc(Cl)nc21] | D | + | IV | NA | NA | NA |
| 200 | 650 | 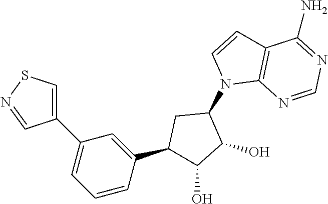 [SMILES: Nc1ncnc2c1ccn2[C@@H]1C[C@H](c2cccc(-c3cnsc3)c2)[C@@H](O)[C@H]1O] | C | + | IV | NA | NA | NA |
| 201 | 392 | 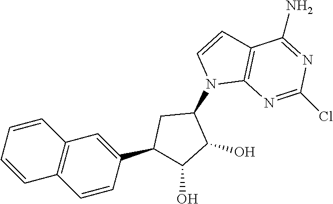 [SMILES: Nc1nc(Cl)nc2c1ccn2[C@@H]1C[C@H](c2ccc3ccccc3c2)[C@@H](O)[C@H]1O] | C | | III | NA | NA | NA |
| 202 | 641 | 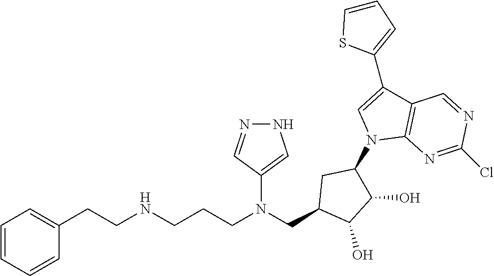 [SMILES: O[C@@H]1[C@@H](CN(CCCNCCc2ccccc2)c2cn[nH]c2)C[C@@H](n2cc(-c3cccs3)c3cnc(Cl)nc32)[C@@H]1O] | A | +++ | II | NA | ** | ## |
| 203 | 623 | 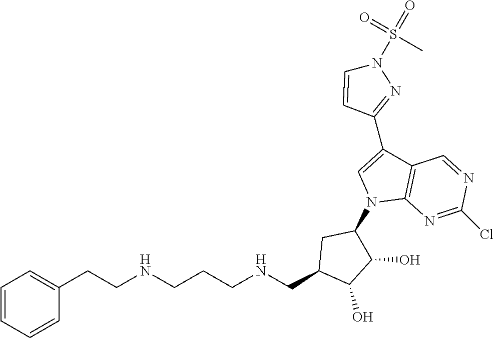 [SMILES: CS(=O)(=O)n1ccc(-c2cn([C@@H]3C[C@H](CNCCCNCCc4ccccc4)[C@@H](O)[C@H]3O)c3nc(Cl)ncc23)n1] | A | +++ | II | NA | *** | ## |
| 204 | 496 | 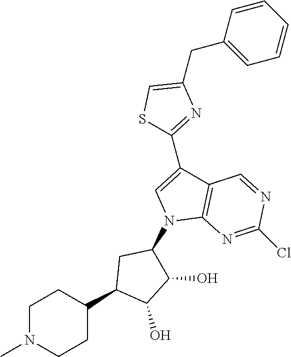 [SMILES: CN1CCC([C@H]2C[C@@H](n3cc(-c4nc(Cc5ccccc5)cs4)c4cnc(Cl)nc43)[C@H](O)[C@@H]2O)CC1] | B | +++ | III | NA | ** | # |
| 205 | 497 | 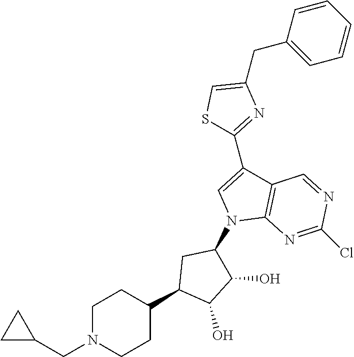 [SMILES: O[C@@H]1[C@H](O)[C@@H](C2CCN(CC3CC3)CC2)C[C@H]1n1cc(-c2nc(Cc3ccccc3)cs2)c2cnc(Cl)nc21] | B | ++ | III | NA | *** | ## |
| 206 | 499 | 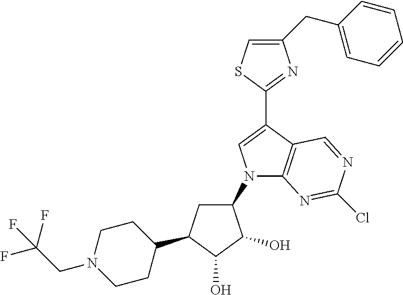 [SMILES: O[C@@H]1[C@H](O)[C@@H](C2CCN(CC(F)(F)F)CC2)C[C@H]1n1cc(-c2nc(Cc3ccccc3)cs2)c2cnc(Cl)nc21] | D | + | IV | NA | NA | NA |
| 207 | 498 | 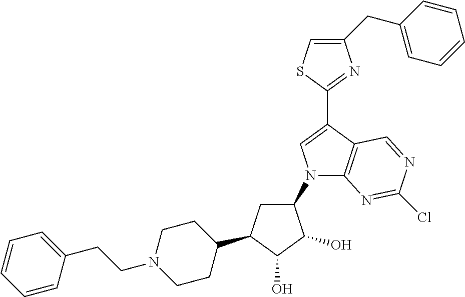 [SMILES: O[C@@H]1[C@H](O)[C@@H](C2CCN(CCc3ccccc3)CC2)C[C@H]1n1cc(-c2nc(Cc3ccccc3)cs2)c2cnc(Cl)nc21] | B | ++ | III | NA | *** | NA |
| 208 | 505 | 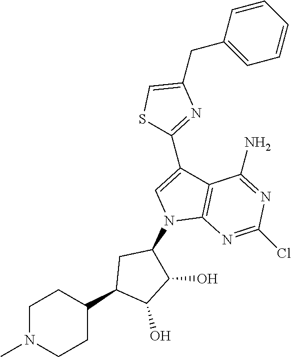 [SMILES: CN1CCC([C@H]2C[C@@H](n3cc(-c4nc(Cc5ccccc5)cs4)c4c(N)nc(Cl)nc43)[C@H](O)[C@@H]2O)CC1] | A | ++++ | II | NA | *** | ## |
| 209 | 585 | 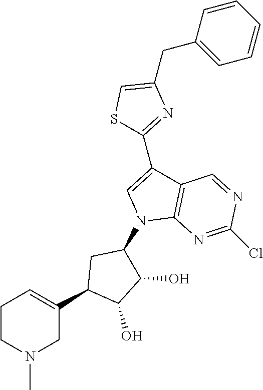 [SMILES: CN1CCC=C([C@H]2C[C@@H](n3cc(-c4nc(Cc5ccccc5)cs4)c4cnc(Cl)nc43)[C@H](O)[C@@H]2O)C1] | C | ++ | III | NA | *** | NA |
| 210 | 586 | 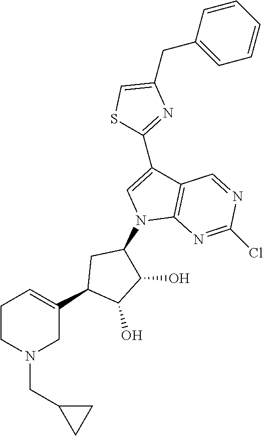 [SMILES: O[C@@H]1[C@H](O)[C@@H](C2=CCCN(CC3CC3)C2)C[C@H]1n1cc(-c2nc(Cc3ccccc3)cs2)c2cnc(Cl)nc21] | C | + | III | NA | NA | NA |
| 211 | 651 | 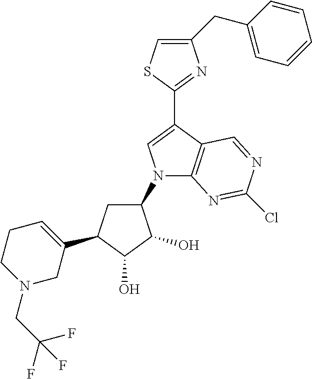 [SMILES: O[C@@H]1[C@H](O)[C@@H](C2=CCCN(CC(F)(F)F)C2)C[C@H]1n1cc(-c2nc(Cc3ccccc3)cs2)c2cnc(Cl)nc21] | D | + | IV | NA | NA | NA |
| 212 | 588 | 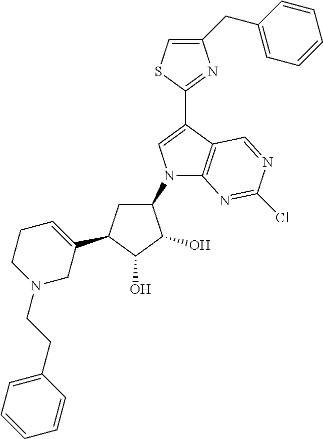 [SMILES: O[C@@H]1[C@H](O)[C@@H](C2=CCCN(CCc3ccccc3)C2)C[C@H]1n1cc(-c2nc(Cc3ccccc3)cs2)c2cnc(Cl)nc21] | D | + | IV | NA | NA | NA |
| 213 | 495 | 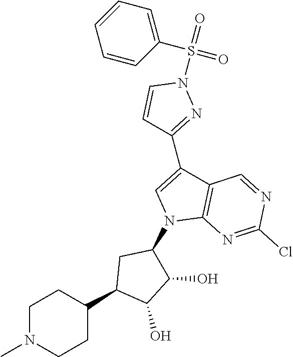 [SMILES: CN1CCC([C@H]2C[C@@H](n3cc(-c4ccn(S(=O)(=O)c5ccccc5)n4)c4cnc(Cl)nc43)[C@H](O)[C@@H]2O)CC1] | B | +++ | III | NA | ** | NA |
| 214 | 397 | 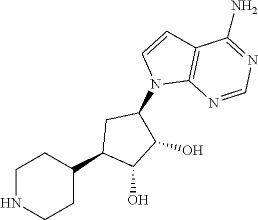 [SMILES: Nc1ncnc2c1ccn2[C@@H]1C[C@H](C2CCNCC2)[C@@H](O)[C@H]1O] | C | ++ | III | NA | NA | NA |
| 215 | 391 | 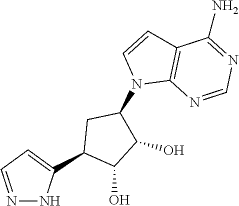 [SMILES: Nc1ncnc2c1ccn2[C@@H]1C[C@H](c2ccn[nH]2)[C@@H](O)[C@H]1O] | D | + | IV | NA | NA | NA |
| 216 | 409 | 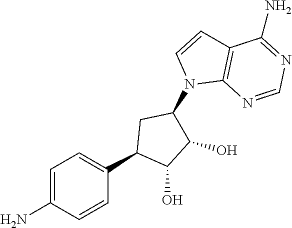 [SMILES: Nc1ccc([C@H]2C[C@@H](n3ccc4c(N)ncnc43)[C@H](O)[C@@H]2O)cc1] | D | + | IV | NA | NA | NA |
| 217 | 405 | 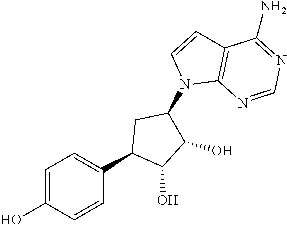 [SMILES: Nc1ncnc2c1ccn2[C@@H]1C[C@H](c2ccc(O)cc2)[C@@H](O)[C@H]1O] | D | ++ | IV | NA | NA | NA |
| 218 | 646 | 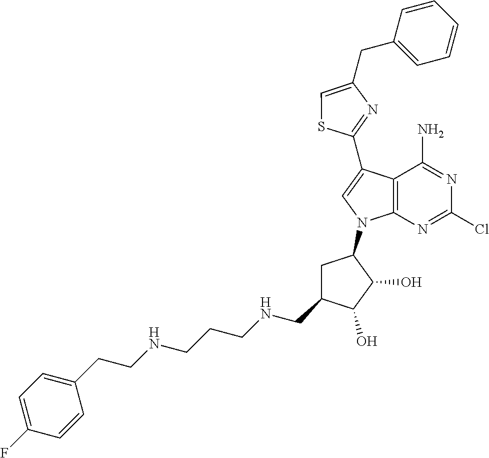 [SMILES: Nc1nc(Cl)nc2c1c(-c1nc(Cc3ccccc3)cs1)cn2[C@@H]1C[C@H](CNCCCNCCc2ccc(F)cc2)[C@@H](O)[C@H]1O] | B | ++++ | II | NA | *** | ## |
| 219 | 587 | 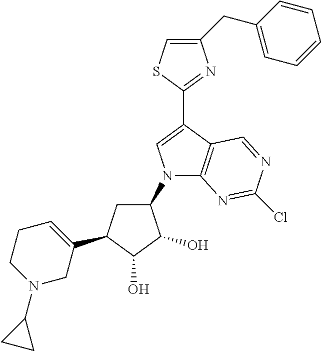 [SMILES: O[C@@H]1[C@H](O)[C@@H](C2=CCCN(C3CC3)C2)C[C@H]1n1cc(-c2nc(Cc3ccccc3)cs2)c2cnc(Cl)nc21] | D | + | IV | NA | NA | NA |
| 220 | 652 | 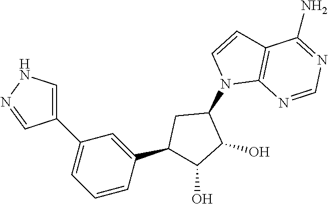 [SMILES: Nc1ncnc2c1ccn2[C@@H]1C[C@H](c2cccc(-c3cn[nH]c3)c2)[C@@H](O)[C@H]1O] | D | + | IV | NA | NA | NA |
| 221 | 644 | 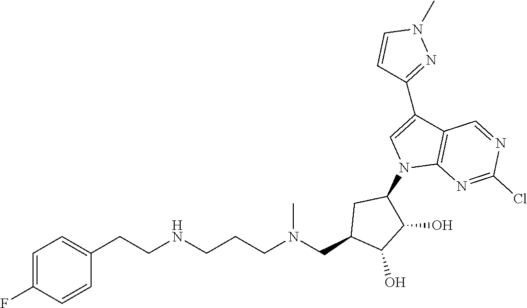 [SMILES: CN(CCCNCCc1ccc(F)cc1)C[C@H]1C[C@@H](n2cc(-c3ccn(C)n3)c3cnc(Cl)nc32)[C@H](O)[C@@H]1O] | D | + | IV | NA | NA | NA |
| 222 | 512 | 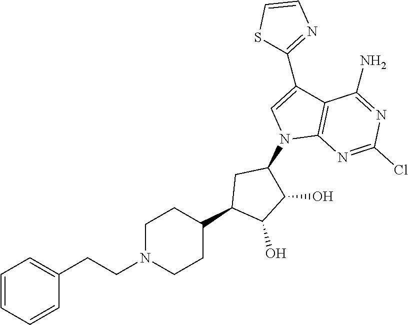 [SMILES: Nc1nc(Cl)nc2c1c(-c1nccs1)cn2[C@@H]1C[C@H](C2CCN(CCc3ccccc3)CC2)[C@@H](O)[C@H]1O] | A | ++++ | II | NA | *** | ## |
| 223 | 504 | 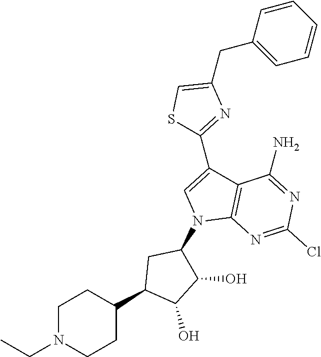 [SMILES: CCN1CCC([C@H]2C[C@@H](n3cc(-c4nc(Cc5ccccc5)cs4)c4c(N)nc(Cl)nc43)[C@H](O)[C@@H]2O)CC1] | A | ++++ | II | NA | *** | ## |
| 224 | 506 | 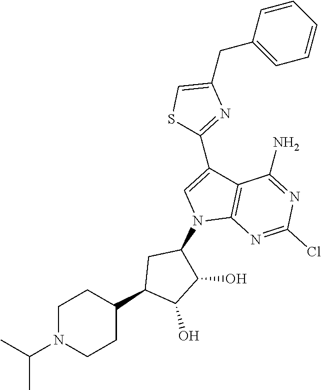 [SMILES: CC(C)N1CCC([C@H]2C[C@@H](n3cc(-c4nc(Cc5ccccc5)cs4)c4c(N)nc(Cl)nc43)[C@H](O)[C@@H]2O)CC1] | A | ++++ | II | NA | *** | ## |
| 225 | 507 | 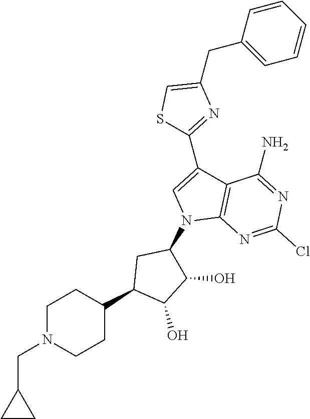 [SMILES: Nc1nc(Cl)nc2c1c(-c1nc(Cc3ccccc3)cs1)cn2[C@@H]1C[C@H](C2CCN(CC3CC3)CC2)[C@@H](O)[C@H]1O] | A | ++++ | II | NA | *** | ### |
| 226 | 492 | 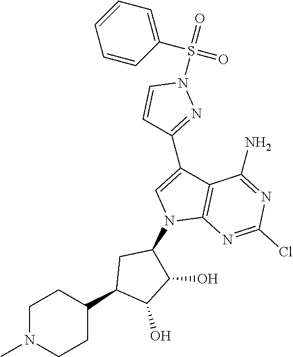 [SMILES: CN1CCC([C@H]2C[C@@H](n3cc(-c4ccn(S(=O)(=O)c5ccccc5)n4)c4c(N)nc(Cl)nc43)[C@H](O)[C@@H]2O)CC1] | A | ++++ | II | NA | *** | NA |
| 227 | 404 | 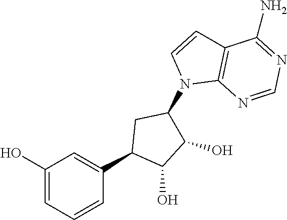 [SMILES: Nc1ncnc2c1ccn2[C@@H]1C[C@H](c2cccc(O)c2)[C@@H](O)[C@H]1O] | D | + | IV | NA | NA | NA |
| 228 | 566 | 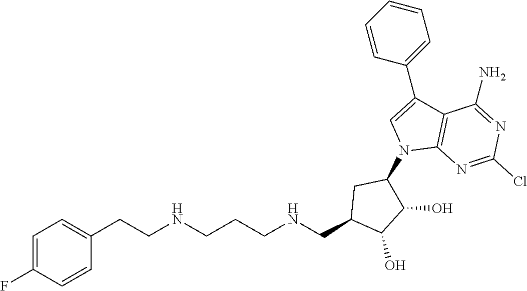 [SMILES: Nc1nc(Cl)nc2c1c(-c1ccccc1)cn2[C@@H]1C[C@H](CNCCCNCCc2ccc(F)cc2)[C@@H](O)[C@H]1O] | A | ++++ | II | NA | ** | NA |
| 229 | 500 | 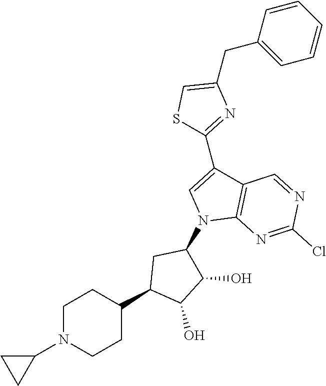 [SMILES: O[C@@H]1[C@H](O)[C@@H](C2CCN(C3CC3)CC2)C[C@H]1n1cc(-c2nc(Cc3ccccc3)cs2)c2cnc(Cl)nc21] | C | ++ | III | NA | NA | NA |
| 230 | 653 | 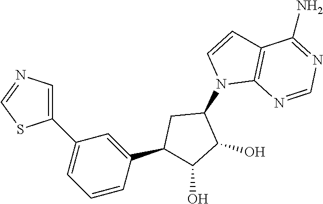 [SMILES: Nc1ncnc2c1ccn2[C@@H]1C[C@H](c2cccc(-c3cncs3)c2)[C@@H](O)[C@H]1O] | D | + | IV | NA | NA | NA |
| 231 | 654 | 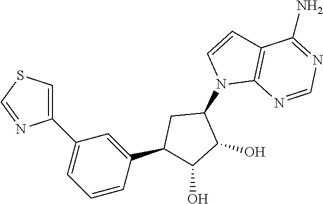 [SMILES: Nc1ncnc2c1ccn2[C@@H]1C[C@H](c2cccc(-c3cscn3)c2)[C@@H](O)[C@H]1O] | D | + | IV | NA | NA | NA |
| 232 | 568 | 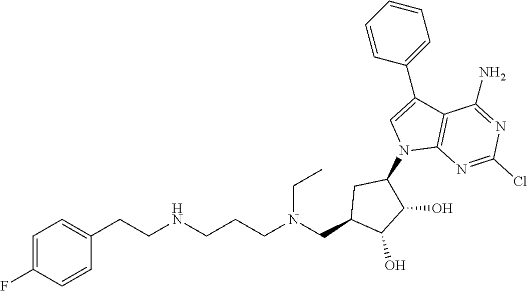 [SMILES: CCN(CCCNCCc1ccc(F)cc1)C[C@H]1C[C@@H](n2cc(-c3ccccc3)c3c(N)nc(Cl)nc32)[C@H](O)[C@@H]1O] | B | ++++ | II | NA | NA | NA |
| 233 | 642 | 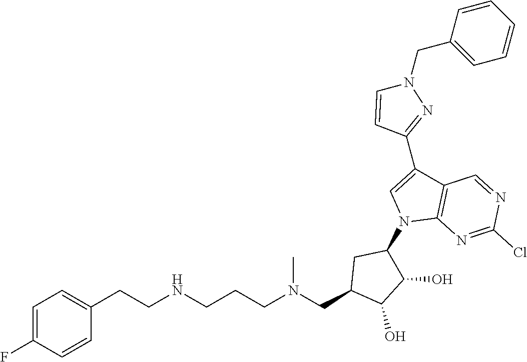 [SMILES: CN(CCCNCCc1ccc(F)cc1)C[C@H]1C[C@@H](n2cc(-c3ccn(Cc4ccccc4)n3)c3cnc(Cl)nc32)[C@H](O)[C@@H]1O] | B | +++ | III | NA | *** | ## |

| | | | | | | | | |
|---|---|---|---|---|---|---|---|---|
| 234 | 508 | 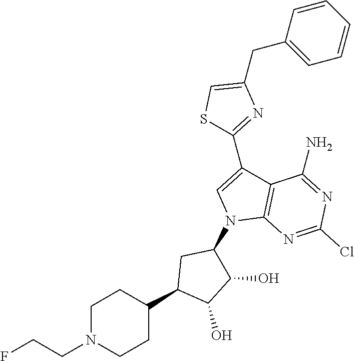 [SMILES: Nc1nc(Cl)nc2c1c(-c1nc(Cc3ccccc3)cs1)cn2[C@@H]1C[C@H](C2CCN(CCF)CC2)[C@@H](O)[C@H]1O] | A | +++ | II | NA | *** | ## |
| 235 | 509 | 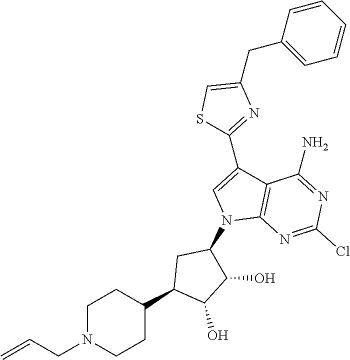 [SMILES: C=CCN1CCC([C@H]2C[C@@H](n3cc(-c4nc(Cc5ccccc5)cs4)c4c(N)nc(Cl)nc43)[C@H](O)[C@@H]2O)CC1] | A | +++ | III | NA | *** | NA |
| 236 | 407 | 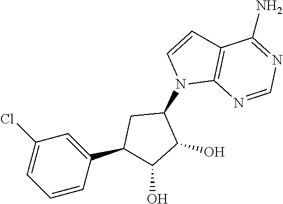 [SMILES: Nc1ncnc2c1ccn2[C@@H]1C[C@H](c2cccc(Cl)c2)[C@@H](O)[C@H]1O] | D | + | IV | NA | | NA |
| 237 | 655 | 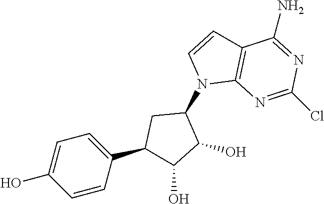 [SMILES: Nc1nc(Cl)nc2c1ccn2[C@@H]1C[C@H](c2ccc(O)cc2)[C@@H](O)[C@H]1O] | A | ++ | II | NA | ** | NA |
| 238 | 656 | 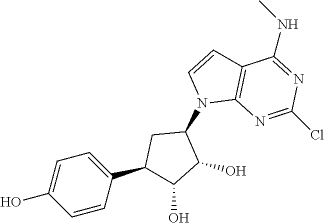 [SMILES: CNc1nc(Cl)nc2c1ccn2[C@@H]1C[C@H](c2ccc(O)cc2)[C@@H](O)[C@H]1O] | B | + | III | NA | ** | NA |
| 239 | 406 | 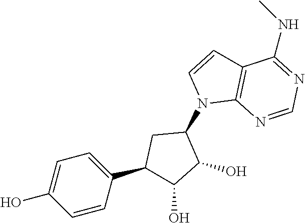 [SMILES: CNc1ncnc2c1ccn2[C@@H]1C[C@H](c2ccc(O)cc2)[C@@H](O)[C@H]1O] | D | + | IV | NA | NA | NA |
| 240 | 512 | 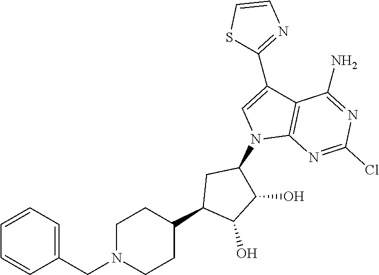 [SMILES: Nc1nc(Cl)nc2c1c(-c1nccs1)cn2[C@@H]1C[C@H](C2CCN(Cc3ccccc3)CC2)[C@@H](O)[C@H]1O] | A | ++++ | II | NA | ** | ## |
| 241 | 513 | 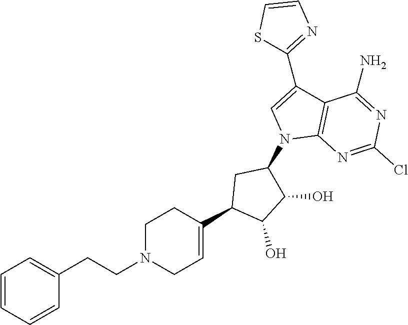 [SMILES: Nc1nc(Cl)nc2c1c(-c1nccs1)cn2[C@@H]1C[C@H](C2=CCN(CCc3ccccc3)CC2)[C@@H](O)[C@H]1O] | A | +++ | II | NA | ** | NA |
| 242 | 596 | 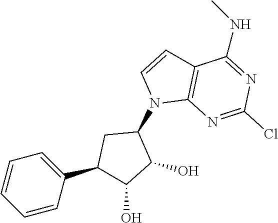 [SMILES: CNc1nc(Cl)nc2c1ccn2[C@@H]1C[C@H](c2ccccc2)[C@@H](O)[C@H]1O] | C | + | III | NA | * | NA |
| 243 | 594 | 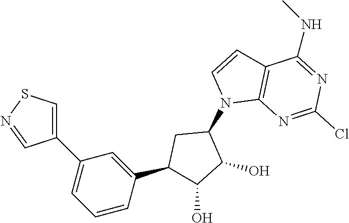 [SMILES: CNc1nc(Cl)nc2c1ccn2[C@@H]1C[C@H](c2cccc(-c3cnsc3)c2)[C@@H](O)[C@H]1O] | C | ++ | III | ** | NA | NA |
| 244 | 569 | 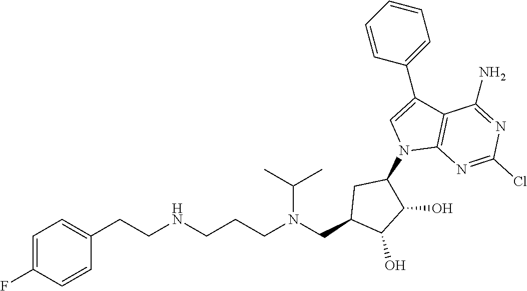 [SMILES: CC(C)N(CCCNCCc1ccc(F)cc1)C[C@H]1C[C@@H](n2cc(-c3ccccc3)c3c(N)nc(Cl)nc32)[C@H](O)[C@@H]1O] | B | ++++ | II | NA | NA | NA |
| 245 | 643 | 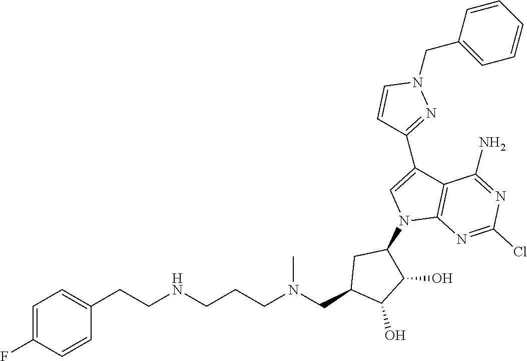 [SMILES: CN(CCCNCCc1ccc(F)cc1)C[C@H]1C[C@@H](n2cc(-c3ccn(Cc4ccccc4)n3)c3c(N)nc(Cl)nc32)[C@H](O)[C@@H]1O] | A | ++++ | II | NA | *** | ## |
| 246 | 645 | 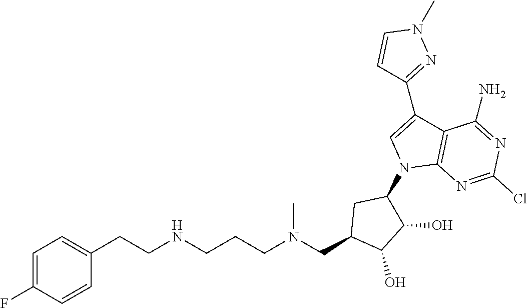 [SMILES: CN(CCCNCCc1ccc(F)cc1)C[C@H]1C[C@@H](n2cc(-c3ccn(C)n3)c3c(N)nc(Cl)nc32)[C@H](O)[C@@H]1O] | B | ++++ | II | NA | NA | NA |
| 247 | 514 | 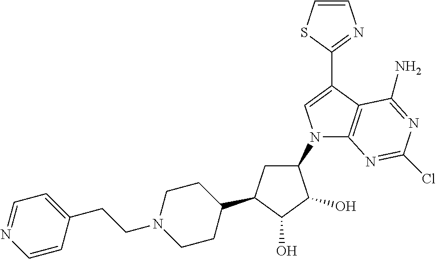 [SMILES: Nc1nc(Cl)nc2c1c(-c1nccs1)cn2[C@@H]1C[C@H](C2CCN(CCc3ccncc3)CC2)[C@@H](O)[C@H]1O] | A | +++ | II | NA | ** | NA |
| 248 | 493 | 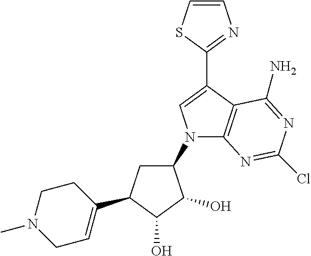 [SMILES: CN1CC=C([C@H]2C[C@@H](n3cc(-c4nccs4)c4c(N)nc(Cl)nc43)[C@H](O)[C@@H]2O)CC1] | A | +++ | II | NA | ** | NA |
| 249 | 523 | 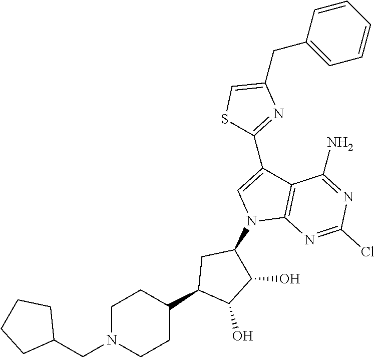 [SMILES: Nc1nc(Cl)nc2c1c(-c1nc(Cc3ccccc3)cs1)cn2[C@@H]1C[C@H](C2CCN(CC3CCCC3)CC2)[C@@H](O)[C@H]1O] | C | ++ | III | NA | NA | ### |
| 250 | 510 | 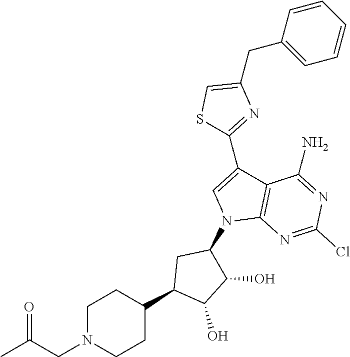 [SMILES: CC(=O)CN1CCC([C@H]2C[C@@H](n3cc(-c4nc(Cc5ccccc5)cs4)c4c(N)nc(Cl)nc43)[C@H](O)[C@@H]2O)CC1] | B | +++ | II | NA | *** | ## |
| 251 | 524 | 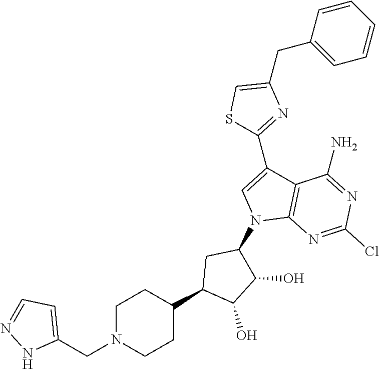 [SMILES: Nc1nc(Cl)nc2c1c(-c1nc(Cc3ccccc3)cs1)cn2[C@@H]1C[C@H](C2CCN(Cc3ccn[nH]3)CC2)[C@@H](O)[C@H]1O] | B | +++ | II | NA | *** | ## |
| 252 | 525 | 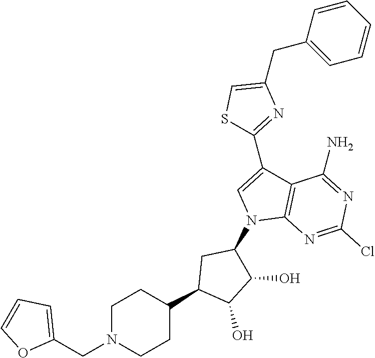 [SMILES: Nc1nc(Cl)nc2c1c(-c1nc(Cc3ccccc3)cs1)cn2[C@@H]1C[C@H](C2CCN(Cc3ccco3)CC2)[C@@H](O)[C@H]1O] | A | +++ | III | NA | *** | ## |
| 253 | 526 | 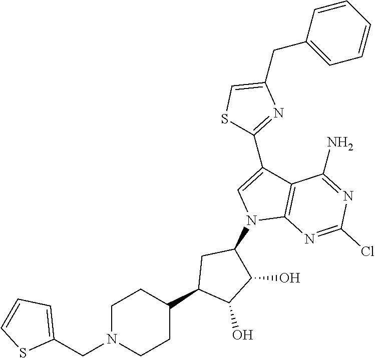 [SMILES: Nc1nc(Cl)nc2c1c(-c1nc(Cc3ccccc3)cs1)cn2[C@@H]1C[C@H](C2CCN(Cc3cccs3)CC2)[C@@H](O)[C@H]1O] | A | +++ | II | NA | *** | ## |
| 254 | 527 | 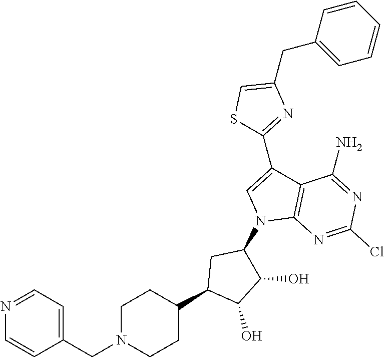 [SMILES: Nc1nc(Cl)nc2c1c(-c1nc(Cc3ccccc3)cs1)cn2[C@@H]1C[C@H](C2CCN(Cc3ccncc3)CC2)[C@@H](O)[C@H]1O] | B | ++ | III | NA | *** | NA |
| 255 | 511 | 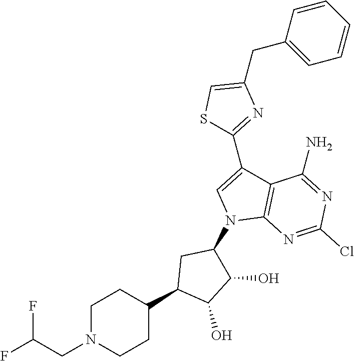 [SMILES: Nc1nc(Cl)nc2c1c(-c1nc(Cc3ccccc3)cs1)cn2[C@@H]1C[C@H](C2CCN(CC(F)F)CC2)[C@@H](O)[C@H]1O] | C | ++ | III | NA | NA | ## |
| 256 | 582 | 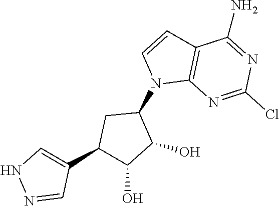 [SMILES: Nc1nc(Cl)nc2c1ccn2[C@@H]1C[C@H](c2cn[nH]c2)[C@@H](O)[C@H]1O] | C | + | III | NA | NA | NA |
| 257 | 658 | 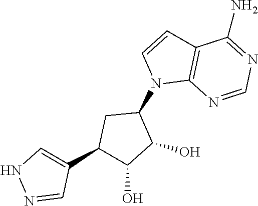 [SMILES: Nc1ncnc2c1ccn2[C@@H]1C[C@H](c2cn[nH]c2)[C@@H](O)[C@H]1O] | D | + | IV | NA | NA | NA |
| 258 | 657 | 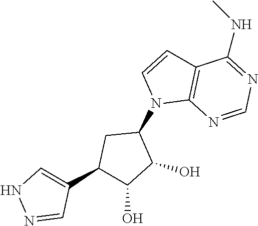 [SMILES: CNc1ncnc2c1ccn2[C@@H]1C[C@H](c2cn[nH]c2)[C@@H](O)[C@H]1O] | D | + | IV | NA | NA | NA |
| 259 | 583 | 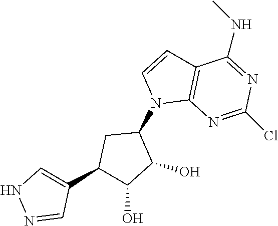 [SMILES: CNc1nc(Cl)nc2c1ccn2[C@@H]1C[C@H](c2cn[nH]c2)[C@@H](O)[C@H]1O] | D | + | IV | NA | NA | NA |
| 260 | 648 | 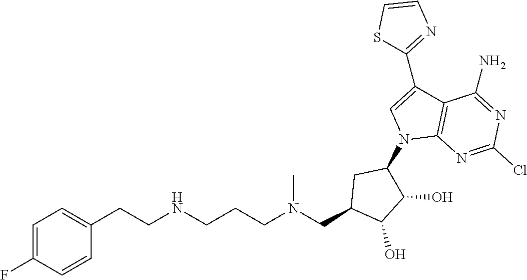 [SMILES: CN(CCCNCCc1ccc(F)cc1)C[C@H]1C[C@@H](n2cc(-c3nccs3)c3c(N)nc(Cl)nc32)[C@H](O)[C@@H]1O] | A | ++++ | II | NA | NA | # |
| 261 | 647 | 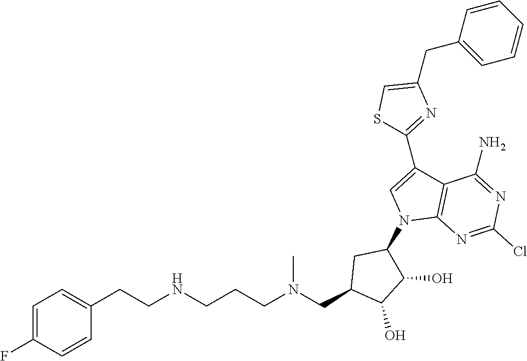 [SMILES: CN(CCCNCCc1ccc(F)cc1)C[C@H]1C[C@@H](n2cc(-c3nc(Cc4ccccc4)cs3)c3c(N)nc(Cl)nc32)[C@H](O)[C@@H]1O] | B | ++++ | III | NA | NA | ## |
| 262 | 597R | 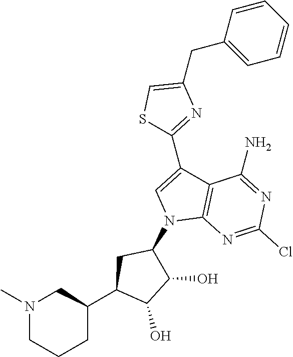 [SMILES: CN1CCC[C@H]([C@H]2C[C@@H](n3cc(-c4nc(Cc5ccccc5)cs4)c4c(N)nc(Cl)nc43)[C@H](O)[C@@H]2O)C1] | A | +++ | II | NA | NA | NA |
| 263 | 597S | 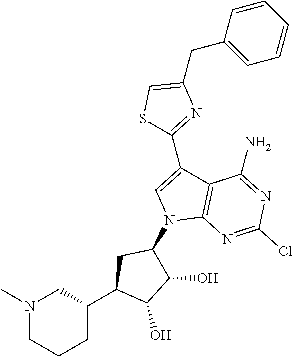 [SMILES: CN1CCC[C@@H]([C@H]2C[C@@H](n3cc(-c4nc(Cc5ccccc5)cs4)c4c(N)nc(Cl)nc43)[C@H](O)[C@@H]2O)C1] | A | +++ | II | NA | NA | NA |
| 264 | 520 | 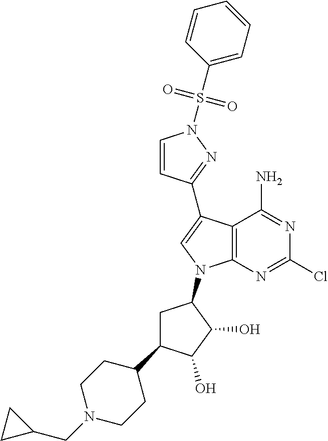 [SMILES: Nc1nc(Cl)nc2c1c(-c1ccn(S(=O)(=O)c3ccccc3)n1)cn2[C@@H]1C[C@H](C2CCN(CC3CC3)CC2)[C@@H](O)[C@H]1O] | A | ++++ | = | NA | NA | ## |
| 265 | 410 | 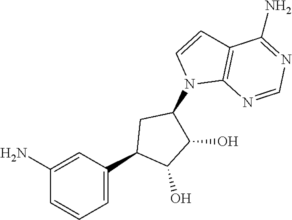 [SMILES: Nc1cccc([C@H]2C[C@@H](n3ccc4c(N)ncnc43)[C@H](O)[C@@H]2O)c1] | D | + | IV | NA | NA | NA |
| 266 | 659 | 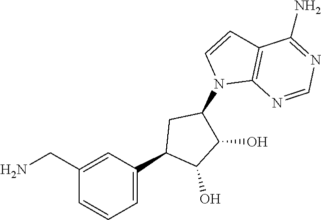 [SMILES: NCc1cccc([C@H]2C[C@@H](n3ccc4c(N)ncnc43)[C@H](O)[C@@H]2O)c1] | C | + | III | NA | NA | NA |
| 267 | 592 | 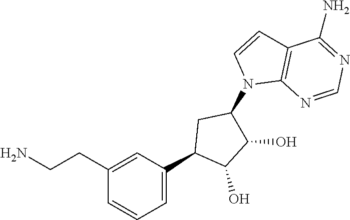 [SMILES: NCCc1cccc([C@H]2C[C@@H](n3ccc4c(N)ncnc43)[C@H](O)[C@@H]2O)c1] | D | + | III | NA | NA | NA |
| 268 | 575 | 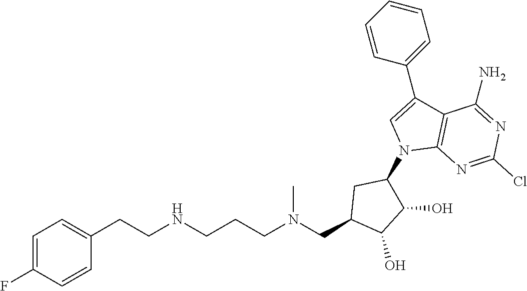 [SMILES: CN(CCCNCCc1ccc(F)cc1)C[C@H]1C[C@@H](n2cc(-c3ccccc3)c3c(N)nc(Cl)nc32)[C@H](O)[C@@H]1O] | A | +++ | II | NA | NA | ## |
| 269 | 519 | 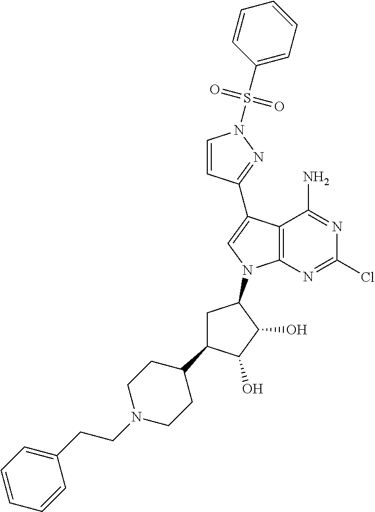 [SMILES: Nc1nc(Cl)nc2c1c(-c1ccn(S(=O)(=O)c3ccccc3)n1)cn2[C@@H]1C[C@H](C2CCN(CCc3ccccc3)CC2)[C@@H](O)[C@H]1O] | A | ++++ | II | NA | NA | ### |
| 270 | 660 | 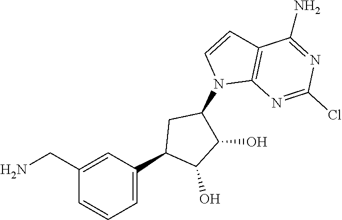 [SMILES: NCc1cccc([C@H]2C[C@@H](n3ccc4c(N)nc(Cl)nc43)[C@H](O)[C@@H]2O)c1] | A | +++ | I | NA | NA | # |
| 271 | 408 | 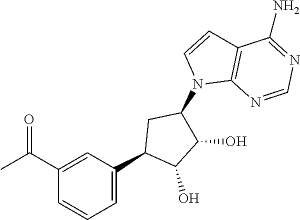 [SMILES: CC(=O)c1cccc([C@H]2C[C@@H](n3ccc4c(N)ncnc43)[C@H](O)[C@@H]2O)c1] | D | + | IV | NA | NA | NA |
| 272 | 392 | 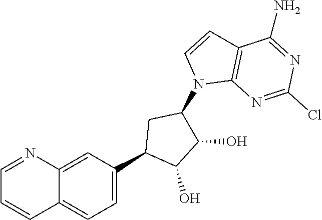 [SMILES: Nc1nc(Cl)nc2c1ccn2[C@@H]1C[C@H](c2ccc3cccnc3c2)[C@@H](O)[C@H]1O] | C | + | III | NA | NA | NA |
| 273 | 661 | 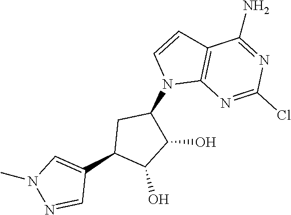 [SMILES: Cn1cc([C@H]2C[C@@H](n3ccc4c(N)nc(Cl)nc43)[C@H](O)[C@@H]2O)cn1] | C | + | III | NA | NA | NA |
| 274 | 584 | 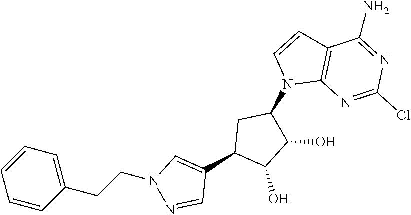 [SMILES: Nc1nc(Cl)nc2c1ccn2[C@@H]1C[C@H](c2cnn(CCc3ccccc3)c2)[C@@H](O)[C@H]1O] | D | + | IV | NA | NA | NA |
| 275 | 590 | 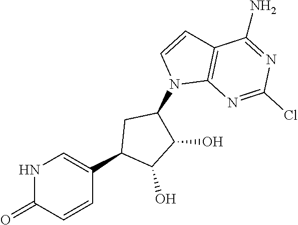 [SMILES: Nc1nc(Cl)nc2c1ccn2[C@@H]1C[C@H](c2ccc(=O)[nH]c2)[C@@H](O)[C@H]1O] | C | + | III | NA | NA | NA |
| 276 | 398 | 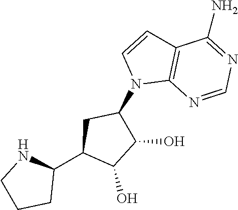 [SMILES: Nc1ncnc2c1ccn2[C@@H]1C[C@H]([C@H]2CCCN2)[C@@H](O)[C@H]1O] | D | + | IV | NA | NA | NA |
| 277 | 399 | 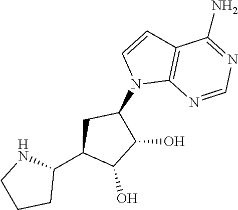 [SMILES: Nc1ncnc2c1ccn2[C@@H]1C[C@H]([C@@H]2CCCN2)[C@@H](O)[C@H]1O] | D | + | IV | NA | NA | NA |
| 278 | 591 | 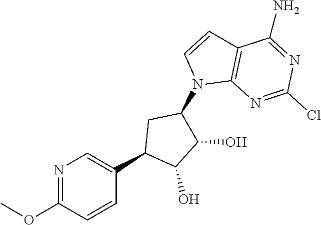 [SMILES: COc1ccc([C@H]2C[C@@H](n3ccc4c(N)nc(Cl)nc43)[C@H](O)[C@@H]2O)cn1] | B | ++ | II | NA | NA | # |
| 279 | 662 | 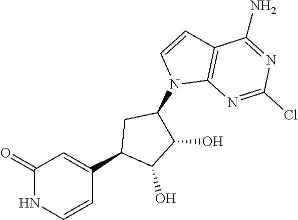 [SMILES: Nc1nc(Cl)nc2c1ccn2[C@@H]1C[C@H](c2cc[nH]c(=O)c2)[C@@H](O)[C@H]1O] | C | + | III | NA | NA | NA |
| 280 | 663 | 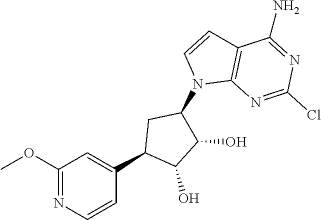 [SMILES: COc1cc([C@H]2C[C@@H](n3ccc4c(N)nc(Cl)nc43)[C@H](O)[C@@H]2O)ccn1] | B | + | II | NA | NA | # |
| 281 | 538 | 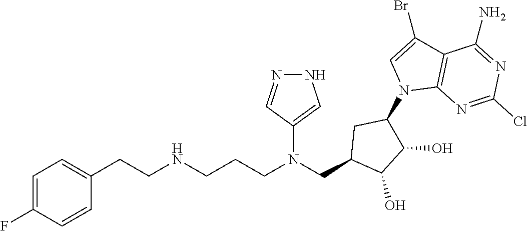 [SMILES: Nc1nc(Cl)nc2c1c(Br)cn2[C@@H]1C[C@H](CN(CCCNCCc2ccc(F)cc2)c2cn[nH]c2)[C@@H](O)[C@H]1O] | A | +++ | II | NA | NA | ## |
| 282 | 595 | 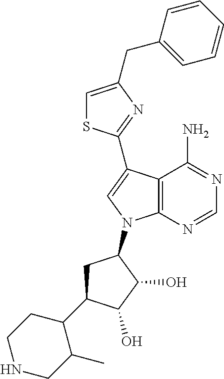 [SMILES: CC1CNCCC1[C@H]1C[C@@H](n2cc(-c3nc(Cc4ccccc4)cs3)c3c(N)ncnc32)[C@H](O)[C@@H]1O] | C | ++ | III | NA | NA | NA |
| 283 | 681 | 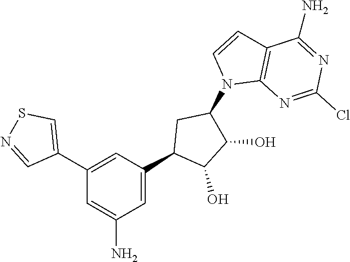 [SMILES: Nc1cc(-c2cnsc2)cc([C@H]2C[C@@H](n3ccc4c(N)nc(Cl)nc43)[C@H](O)[C@@H]2O)c1] | B | ++ | II | NA | NA | NA |
| 284 | 555 | 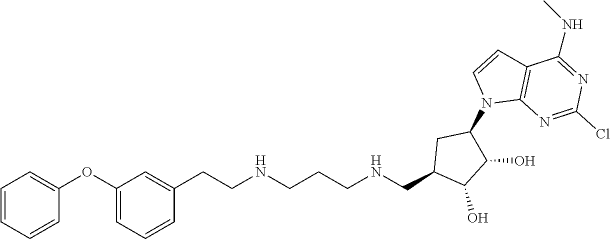 [SMILES: CNc1nc(Cl)nc2c1ccn2[C@@H]1C[C@H](CNCCCNCCc2cccc(Oc3ccccc3)c2)[C@@H](O)[C@H]1O] | B | +++ | II | NA | NA | ## |
| 285 | 664 | 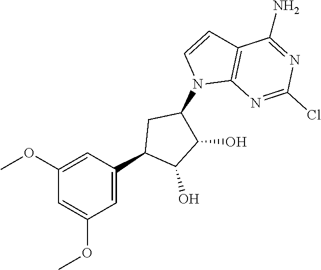 [SMILES: COc1cc(OC)cc([C@H]2C[C@@H](n3ccc4c(N)nc(Cl)nc43)[C@H](O)[C@@H]2O)c1] | B | ++ | II | NA | NA | # |
| 286 | 682 | 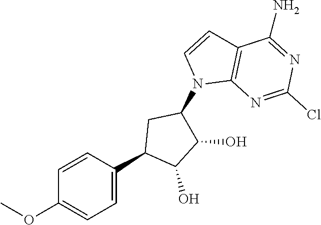 [SMILES: COc1ccc([C@H]2C[C@@H](n3ccc4c(N)nc(Cl)nc43)[C@H](O)[C@@H]2O)cc1] | B | ++ | II | NA | NA | # |
| 287 | 564 | 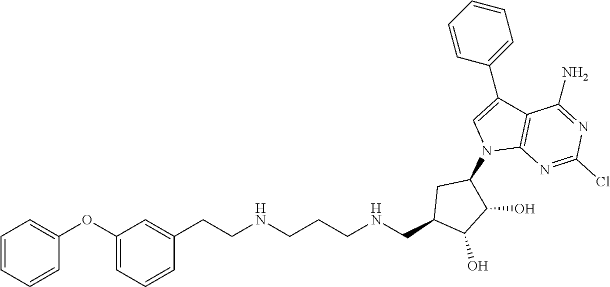 [SMILES: Nc1nc(Cl)nc2c1c(-c1ccccc1)cn2[C@@H]1C[C@H](CNCCCNCCc2cccc(Oc3ccccc3)c2)[C@@H](O)[C@H]1O] | B | ++++ | III | NA | NA | ## |
| 288 | 397 | 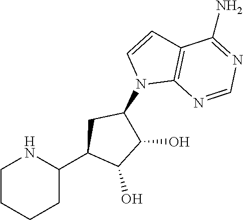 [SMILES: Nc1ncnc2c1ccn2[C@@H]1C[C@H](C2CCCCN2)[C@@H](O)[C@H]1O] | D | + | IV | NA | NA | NA |
| 289 | 500 | 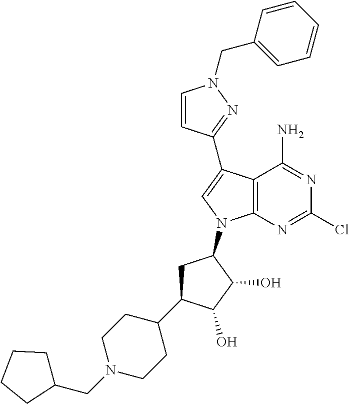 [SMILES: Nc1nc(Cl)nc2c1c(-c1ccn(Cc3ccccc3)n1)cn2[C@@H]1C[C@H](C2CCN(CC3CCCC3)CC2)[C@@H](O)[C@H]1O] | A | ++++ | II | NA | NA | ## |
| 290 | 665 | 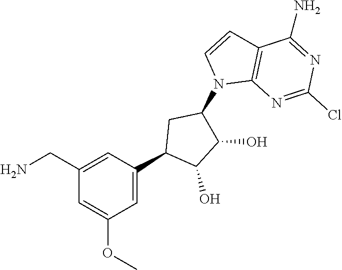 [SMILES: COc1cc(CN)cc([C@H]2C[C@@H](n3ccc4c(N)nc(Cl)nc43)[C@H](O)[C@@H]2O)c1] | A | +++ | II | NA | NA | # |
| 291 | 672 | 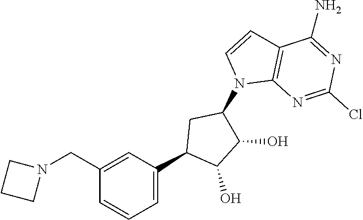 [SMILES: Nc1nc(Cl)nc2c1ccn2[C@@H]1C[C@H](c2cccc(CN3CCC3)c2)[C@@H](O)[C@H]1O] | B | ++ | II | NA | NA | # |
| 292 | 677 | 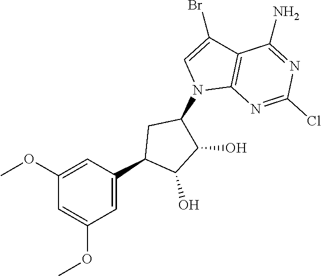 [SMILES: COc1cc(OC)cc([C@H]2C[C@@H](n3cc(Br)c4c(N)nc(Cl)nc43)[C@H](O)[C@@H]2O)c1] | B | ++ | III | NA | NA | # |
| 293 | 678 | 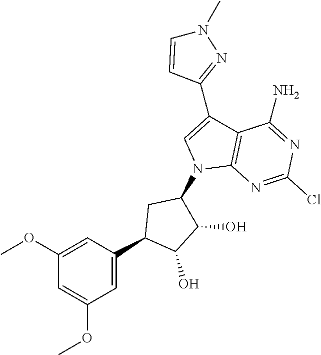 [SMILES: COc1cc(OC)cc([C@H]2C[C@@H](n3cc(-c4ccn(C)n4)c4c(N)nc(Cl)nc43)[C@H](O)[C@@H]2O)c1] | C | ++ | III | NA | NA | ## |
| 294 | 666 | 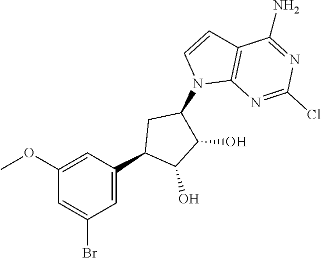 [SMILES: COc1cc(Br)cc([C@H]2C[C@@H](n3ccc4c(N)nc(Cl)nc43)[C@H](O)[C@@H]2O)c1] | B | ++ | II | NA | NA | # |
| 295 | 670 | 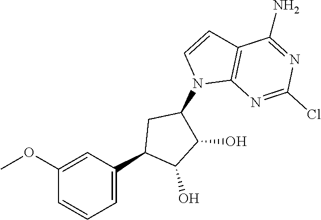 [SMILES: COc1cccc([C@H]2C[C@@H](n3ccc4c(N)nc(Cl)nc43)[C@H](O)[C@@H]2O)c1] | A | ++ | II | NA | NA | # |
| 296 | 547 | 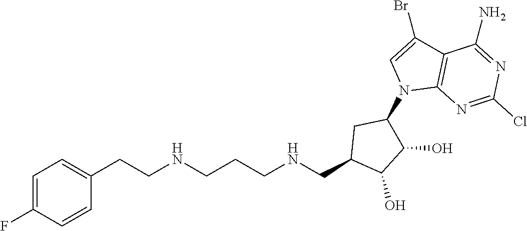 [SMILES: Nc1nc(Cl)nc2c1c(Br)cn2[C@@H]1C[C@H](CNCCCNCCc2ccc(F)cc2)[C@@H](O)[C@H]1O] | A | ++++ | II | NA | NA | ## |
| 297 | 679 | 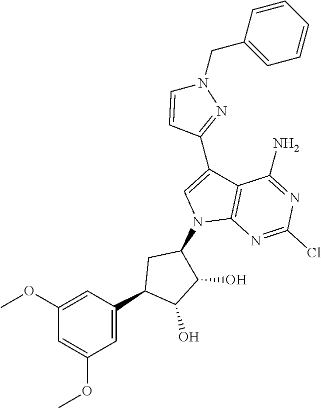 [SMILES: COc1cc(OC)cc([C@H]2C[C@@H](n3cc(-c4ccn(Cc5ccccc5)n4)c4c(N)nc(Cl)nc43)[C@H](O)[C@@H]2O)c1] | C | + | III | NA | NA | ## |
| 298 | 676 | 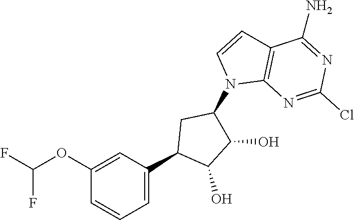 [SMILES: Nc1nc(Cl)nc2c1ccn2[C@@H]1C[C@H](c2cccc(OC(F)F)c2)[C@@H](O)[C@H]1O] | B | + | III | NA | NA | # |
| 299 | 521R | 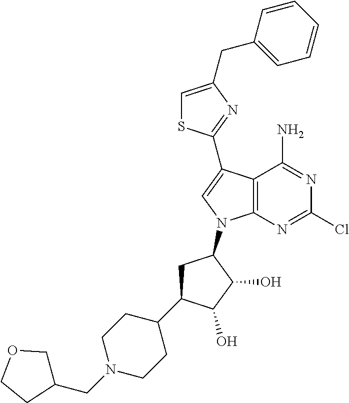 [SMILES: Nc1nc(Cl)nc2c1c(-c1nc(Cc3ccccc3)cs1)cn2[C@@H]1C[C@H](C2CCN(CC3CCOC3)CC2)[C@@H](O)[C@H]1O] | B | +++ | III | NA | NA | ## |
| 300 | 521S | 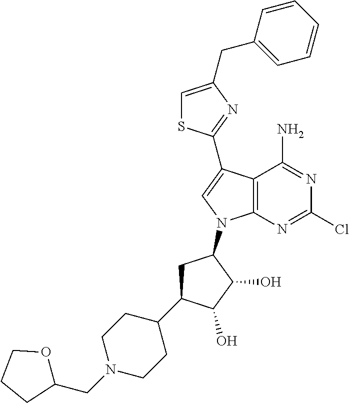 [SMILES: Nc1nc(Cl)nc2c1c(-c1nc(Cc3ccccc3)cs1)cn2[C@@H]1C[C@H](C2CCN(CC3CCCO3)CC2)[C@@H](O)[C@H]1O] | A | +++ | II | NA | NA | ### |
| 301 | 667 | 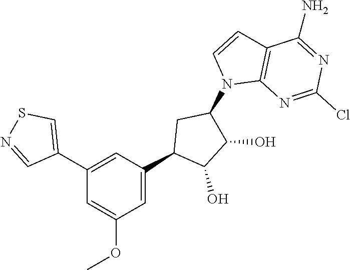 [SMILES: COc1cc(-c2cnsc2)cc([C@H]2C[C@@H](n3ccc4c(N)nc(Cl)nc43)[C@H](O)[C@@H]2O)c1] | B | ++ | III | NA | NA | # |
| 302 | 668 | 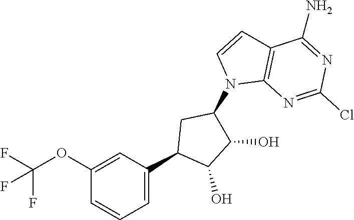 [SMILES: Nc1nc(Cl)nc2c1ccn2[C@@H]1C[C@H](c2cccc(OC(F)(F)F)c2)[C@@H](O)[C@H]1O] | C | + | III | NA | NA | # |
| 303 | 543 | 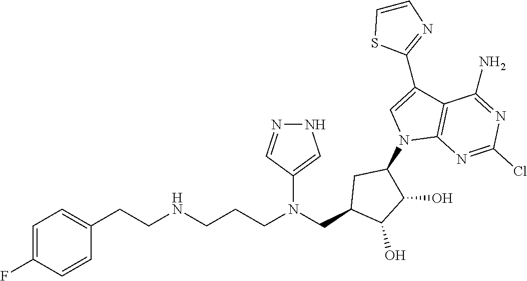 [SMILES: Nc1nc(Cl)nc2c1c(-c1nccs1)cn2[C@@H]1C[C@H](CN(CCCNCCc2ccc(F)cc2)c2cn[nH]c2)[C@@H](O)[C@H]1O] | A | ++++ | II | NA | NA | ### |
| 304 | 673 | 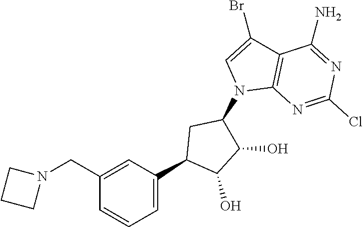 [SMILES: Nc1nc(Cl)nc2c1c(Br)cn2[C@@H]1C[C@H](c2cccc(CN3CCC3)c2)[C@@H](O)[C@H]1O] | A | +++ | II | NA | NA | # |
| 305 | 674 | 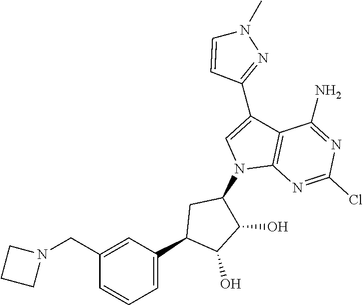 [SMILES: Cn1ccc(-c2cn([C@@H]3C[C@H](c4cccc(CN5CCC5)c4)[C@@H](O)[C@H]3O)c3nc(Cl)nc(N)c23)n1] | A | +++ | II | NA | NA | # |
| 306 | 675 | 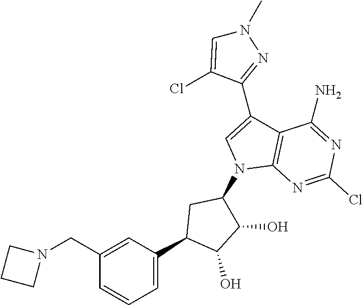 [SMILES: Cn1cc(Cl)c(-c2cn([C@@H]3C[C@H](c4cccc(CN5CCC5)c4)[C@@H](O)[C@H]3O)c3nc(Cl)nc(N)c23)n1] | A | +++ | II | NA | NA | ## |
| 307 | 581 | 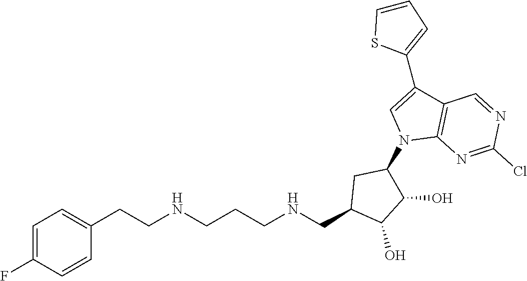 [SMILES: O[C@@H]1[C@@H](CNCCCNCCc2ccc(F)cc2)C[C@@H](n2cc(-c3cccs3)c3cnc(Cl)nc32)[C@@H]1O] | A | ++ | III | NA | NA | ## |
| 308 | 669 | 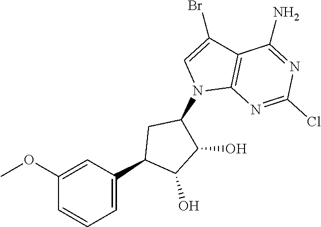 [SMILES: COc1cccc([C@H]2C[C@@H](n3cc(Br)c4c(N)nc(Cl)nc43)[C@H](O)[C@@H]2O)c1] | B | ++ | III | NA | NA | # |
| 309 | 671 | 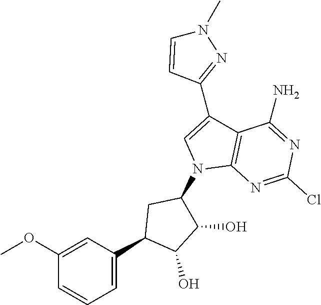 [SMILES: COc1cccc([C@H]2C[C@@H](n3cc(-c4ccn(C)n4)c4c(N)nc(Cl)nc43)[C@H](O)[C@@H]2O)c1] | B | ++ | III | NA | NA | # |
| 310 | 393 | 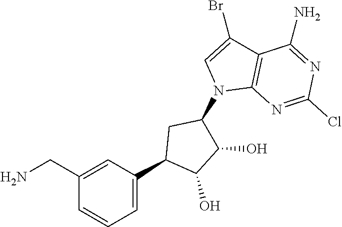 [SMILES: NCc1cccc([C@H]2C[C@@H](n3cc(Br)c4c(N)nc(Cl)nc43)[C@H](O)[C@@H]2O)c1] | A | +++ | II | NA | NA | # |
| 311 | 680 | 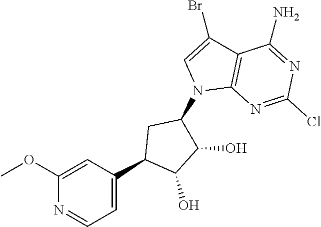 [SMILES: COc1cc([C@H]2C[C@@H](n3cc(Br)c4c(N)nc(Cl)nc43)[C@H](O)[C@@H]2O)ccn1] | B | ++ | II | NA | NA | # |
| 312 | 401 | 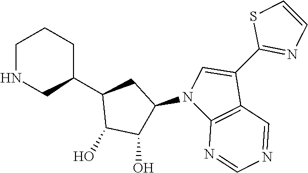 [SMILES: O[C@@H]1[C@H](O)[C@@H]([C@@H]2CCCNC2)C[C@H]1n1cc(-c2nccs2)c2cncnc21] | NA | + | NA | NA | NA | NA |
| 313 | 402 | 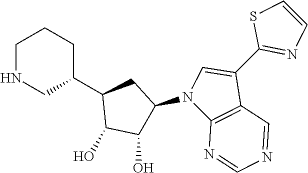 [SMILES: O[C@@H]1[C@H](O)[C@@H]([C@H]2CCCNC2)C[C@H]1n1cc(-c2nccs2)c2cncnc21] | NA | + | NA | NA | NA | NA |
| 314 | 403 | 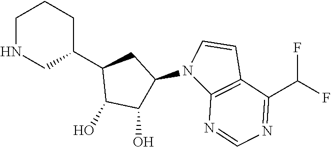 [SMILES: O[C@@H]1[C@H](O)[C@@H]([C@H]2CCCNC2)C[C@H]1n1ccc2c(C(F)F)ncnc21] | NA | + | NA | NA | NA | NA |

1) The indication in brackets in the column “Name” in the above Table 1 refers to the name of the respective compounds of the priority application.
2) The abbreviation NA in the above Table 1 stands for not available.

### Further Cell Proliferation Assays

Compound no. 1 (Name 287) was tested in further cell proliferation assays using different cancer cell lines. Cell proliferation was determined using X-Celligence RTCA system (Roche). For real-time recording of CAL-29, VM-CUB1, SW480, SKBR3, HT-1376, PC-3M, A549, UM-UC-3, DU145 and 22RV1, between 2500 and 20000 cells/well (depending on the cell type) were seeded in 16 well E-plates (Roche). Cells were seeded in presence of DMSO or Compound no. 1 (Name 287) 24 h before seeding in E-plates. Cell indices were automatically recorded every 15 minutes. Relative velocities represent the change of the cell index over time. The ECso values of compound no. 1 (Name 287) in different cancer cell lines are shown in Table 2

TABLE 2
EC₅₀ of compound no. 1 (Name 287) in different cancer cell lines

| Cell line | EC₅₀ (μM) |
|---|---|
| CAL-29 | 6.45 |
| VM-CUB1 | 6.17 |
| SW480 | 9.12 |
| SKBR3 | 6.60 |
| HT-1376 | 12.6 |
| PC-3M* | 15.8 |
| A549 | 12.0 |
| UM-UC-3 | 17.0 |
| DU145* | 19.0 |
| 22RV1* | 16.6 |

*Castration resistant prostate cancer cells

1. A compound of formula (I) 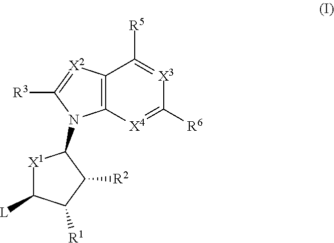
or a salt, stereoisomer, tautomer or N-oxide thereof, 1-1
wherein
X¹ is CH₂, or N;
X² is CR⁴ or N;
X³ is CH or N;
X⁴ is CH, or N;
R¹, R² are independently of each other selected from H, halogen and OH;
R³ is H, or C₁-C₄-alkyl;
R⁴ is H, CN, halogen, C₁-C₄-alkyl, C₂-C₄-alkenyl, phenyl, or a 3-to 6-membered saturated, partially or fully unsaturated, or aromatic carbocyclyl, or heterocyclyl, wherein the aforementioned heterocyclic ring comprises one or more, same or different heteroatoms selected from O, N and S, wherein said N- and/or S-atoms are independently oxidized or non-oxidized, and wherein each substitutable carbon or heteroatom in the aforementioned groups is independently unsubstituted or substituted with one or more same or different substituents R^{x};
R⁵ is H, C₁-C₄-alkyl, C₁-C₄-haloalkyl, or NRªR^{b}, or
R⁴ and R⁵ together with the atoms to which they are bonded form a 7-to 10-membered partially or fully unsaturated carbocyclyl or heterocyclyl, wherein the aforementioned heterocyclic ring comprises one or more, same or different heteroatoms selected from O, N and S, wherein said N- and/or S-atoms are independently oxidized or non-oxidized, and wherein each substitutable carbon or heteroatom in the aforementioned groups is independently unsubstituted or substituted with one or more, same or different substituents R^{x};
R⁶ is H, halogen, C₁-C₄-alkyl, NR^{a}R^{b}, or OR^{e};
wherein
L (i) is a 4-to 6-membered saturated, partially or fully unsaturated or aromatic heterocyclyl, wherein the aforementioned heterocyclic ring comprises one or more, same or different heteroatoms selected from O, N and S, and wherein said N- and/or S-atoms are independently oxidized or non-oxidized, and wherein each substitutable carbon or heteroatom in the aforementioned groups is independently unsubstituted or substituted with one or more, same or different substituents R^{Y}, or
a 5- or 6-membered aromatic carbocyclyl, wherein the aforementioned carbocyclic ring is independently unsubstituted or substituted with one or more, same or different substituents R^{Y}; or
a 7-to 10-membered aromatic carbobicyclyl or heterobicyclyl, wherein the aforementioned heterobicyclic ring comprises one or more, same or different heteroatoms selected from O, N and S, and wherein said N- and/or S-atoms are independently oxidized or non-oxidized, and wherein each substitutable carbon or heteroatom in the aforementioned groups is independently unsubstituted or substituted with one or more, same or different substituents R^{Y}; or
(ii) is selected from 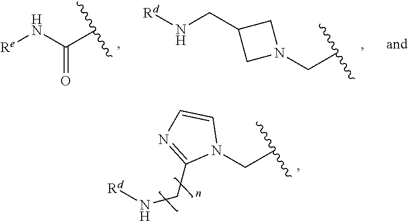
wherein
each substitutable carbon atom in the aforementioned groups is independently unsubstituted or substituted with one or more, same or different substituents selected from halogen, or two substituents together form C(═O);
wherein
R^{a}, R^{b} are independently of each other selected from H, C₁-C₄-alkyl and phenyl;
R^{c} is H, C₁-C₄-alkyl, NR^{a}R^{b}, NR^{a}R^{b}-C₁-C₄-alkyl, benzyl, or a 5- or 6-membered saturated heterocyclyl, wherein the aforementioned heterocyclic ring comprises one or more, same or different heteroatoms selected from O, N and S, and wherein said N- and/or S-atoms are independently oxidized or non-oxidized;
R^{d} is benzophenone-C₁-C₄-alkyl, phenoxybenzene-C₁-C₄-alkyl, N-methyl-diphenylamine-C₁-C₄-alkyl, or diphenylsulfide-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae 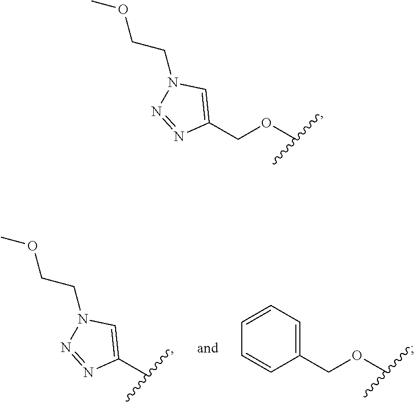
R^{e} is a 5- or 6-membered saturated heterocyclyl, wherein said heterocyclic ring comprises one or more, same or different heteroatoms selected from O, N and S, wherein said N- and/or S-atoms are independently oxidized or non-oxidized, and wherein each substitutable carbon or heteroatom in the aforementioned groups is independently unsubstituted or substituted with one or more, same or different substituents R^{x};
R^{g} is H, C(═O) R^{e}, phenyl-C₁-C₄-alkyl, benzophenone-C₁-C₄-alkyl, phenoxybenzene-C₁-C₄-alkyl, N-methyl-diphenylamine-C₁-C₄-alkyl, diphenylsulfide-C₁-C₄-alkyl, or naphthalene-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae 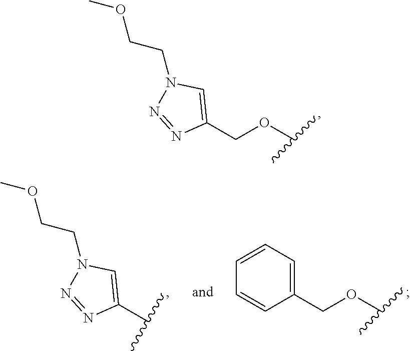
R^{h} is C₁-C₄-alkyl, or phenyl;
Rⁱ is C₁-C₄-alkyl, or phenyl;
R^{x} is C₁-C₄-alkyl, C(═O)R^{c}, S(═O)₂Rh, ORⁱ, or phenyl-C₁-C₄-alkyl;
R^{Y} is halogen, NH₂, OH, C₁-C₄-alkyl, C₁-C₄-haloalkyl, C₂-C₆-alkenyl, C₁-C₄-alkoxy, C₁-C₄-haloalkoxy, cyclopropyl, cyclopropyl-C₁-C₄-alkyl, phenyl-C₁-C₄-alkyl, two R^{Y} form-O, or a 4-to 6-membered saturated, partially or fully unsaturated or aromatic carbocyclyl, carbocyclyl-C₁-C₄-alkyl, heterocyclyl or heterocyclyl-C₁-C₄-alkyl, wherein the aforementioned heterocyclic rings comprise one or more, same or different heteroatoms selected from O, N and S, wherein said N- and/or S-atoms are independently oxidized or non-oxidized, and wherein each substitutable carbon in the aforementioned groups is independently unsubstituted or substituted with one or more, same or different substituents selected from NH₂, or two substituents form ═O; and
n is 1, 2, 3 or 4;
with the proviso that if X² is N or CR⁴, wherein R⁴ is H, then R⁵ is not H or NH₂.

2. The compound according to claim 1, wherein
R^{x} is C₁-C₄-alkyl, C(═O) R^{e}, or phenyl-C₁-C₄-alkyl.

3. The compound according to claim 1, wherein
X¹ is CH₂;
X² is CR⁴;
X³ is N; and
X⁴ is N;
with the proviso that if X² is CR⁴, wherein R⁴ is H, then R⁵ is not H or NH₂.

4. The compound according to claim 1, wherein
R¹, R² are OH;
R³ is H;
R⁴ is H, halogen, C₁-C₄-alkyl or
a 5-membered fully unsaturated or aromatic heterocyclyl, wherein the aforementioned heterocyclic ring comprises one or more, same or different heteroatoms selected from O, N and S, wherein said N- and/or S-atoms are independently oxidized or non-oxidized, and wherein each substitutable carbon or heteroatom in the aforementioned groups is independently unsubstituted or substituted with one or more same or different substituents R^{x};
R⁵ is H, or NR^{a}R^{b}; or
R⁴ and R⁵ together with the atoms to which they are bonded form a 7-to 10-membered partially or fully unsaturated carbocyclyl or heterocyclyl, wherein the aforementioned heterocyclic ring comprises one or more, same or different heteroatoms selected from O, N and S, wherein said N- and/or S-atoms are independently oxidized or non-oxidized, and wherein each substitutable carbon or heteroatom in the aforementioned groups is independently unsubstituted or substituted with one or more, same or different substituents R^{x};
R⁶ is H, or halogen;
with the proviso that if X² is CR⁴, wherein R⁴ is H, then R⁵ is not H or NH₂.

5. The compound according to claim 1, wherein
L (i) is a 4-to 6-membered saturated, partially or fully unsaturated or aromatic heterocyclyl, wherein the aforementioned heterocyclic ring comprises one or more, same or different heteroatoms selected from O, N and S, and wherein said N- and/or S-atoms are independently oxidized or non-oxidized, and wherein each substitutable carbon or heteroatom in the aforementioned groups is independently unsubstituted or substituted with one or more, same or different substituents R^{Y}, or
a 5- or 6-membered aromatic carbocyclyl, wherein the aforementioned carbocyclic ring is independently unsubstituted or substituted with one or more, same or different substituents R^{Y}.

6. A pharmaceutical composition comprising a pharmaceutically effective amount of the compound according to claim 1 and a pharmaceutically acceptable carrier, diluent, or excipient.

7. A method of treating a patient comprising administering the compound according to claim 1.

8. A method of treating cancer, the method comprising administering the compound according to claim 1 wherein the cancer is selected from the group consisting of prostate cancer, breast cancer, ovarian cancer, colon cancer, glioblastoma, lung cancer, neuroblastoma, osteosarcoma, liposarcoma, colorectal cancer, rectal adenocarcinoma, mesothelioma, endometrium adenocarcinoma, leukemia, erythroleukemia, medulloblastoma, astrocytoma, Ewing sarcoma, myelodysplastic syndrome (MDS), diffuse large B-cell lymphoma, myelogenic leukemia, myeloid leukemia, acute monocytic leukemia, gallbladder carcinoma, cecum adenocarcinoma, gastric adenocarcinoma, stomach adenocarcinoma, renal cell carcinoma, bladder carcinoma, melanoma, cervical squamous cell carcinoma, pancreatic carcinoma, chondrosarcoma, duodenal adenocarcinoma, rhabdomyosarcoma, hepatocellular carcinoma and uterine adenocarcinoma.

9. The method according to claim 8, wherein said cancer is selected from the group consisting of prostate cancer, breast cancer, ovarian cancer, colon cancer, glioblastoma, lung cancer, neuroblastoma, colorectal cancer and bladder carcinoma.

10. A method of treating prostate cancer, the method comprising administering a compound of formula (I) 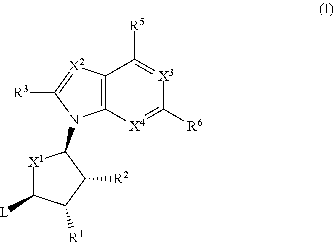
or a salt, stereoisomer, tautomer or N-oxide thereof,
wherein
X¹ is CH₂, N or O;
X² is CR⁴, or N;
X³ is CH, or N;
X⁴ is CH, or N;
R¹, R² are independently of each other selected from H, halogen and OH;
R³ is H, or C₁-C₄-alkyl;
R⁴ is H, CN, halogen, C₁-C₄-alkyl, C₂-C₄-alkenyl, phenyl, or a 3-to 6-membered saturated, partially or fully unsaturated, or aromatic carbocyclyl, or heterocyclyl, wherein the aforementioned heterocyclic ring comprises one or more, same or different heteroatoms selected from O, N and S, wherein said N- and/or S-atoms are independently oxidized or non-oxidized, and wherein each substitutable carbon or heteroatom in the aforementioned groups is independently unsubstituted or substituted with one or more same or different substituents R^{x};
R⁵ is H, halogen, C₁-C₄-alkyl, C₁-C₄-haloalkyl, or NR^{a}R^{b}, or R⁴ and R⁵ together with the atoms to which they are bonded form a 7-to 10-membered partially or fully unsaturated carbocyclyl or heterocyclyl, wherein the aforementioned heterocyclic ring comprises one or more, same or different heteroatoms selected from O, N and S, wherein said N- and/or S-atoms are independently oxidized or non-oxidized, and wherein each substitutable carbon or heteroatom in the aforementioned groups is independently unsubstituted or substituted with one or more, same or different substituents R^{x};
R⁶ is H, halogen, C₁-C₄-alkyl, NR^{a}R^{b}, or OR^{c};
wherein
L (i) is a 4-to 6-membered saturated, partially or fully unsaturated or aromatic heterocyclyl, or heterocyclyl-C₁-C₄-alkyl, wherein the aforementioned heterocyclic rings comprise one or more, same or different heteroatoms selected from O, N and S, and wherein said N- and/or S-atoms are independently oxidized or non-oxidized, and wherein each substitutable carbon or heteroatom in the aforementioned groups is independently unsubstituted or substituted with one or more, same or different substituents R^{Y}, or
a 5- or 6-membered aromatic carbocyclyl, wherein the aforementioned carbocyclic ring is independently unsubstituted or substituted with one or more, same or different substituents R^{Y}; or
a 7-to 10-membered aromatic carbobicyclyl or heterobicyclyl, wherein the aforementioned heterobicyclic ring comprises one or more, same or different heteroatoms selected from O, N and S, and wherein said N- and/or S-atoms are independently oxidized or non-oxidized, and wherein each substitutable carbon or heteroatom in the aforementioned groups is independently unsubstituted or substituted with one or more, same or different substituents R^{Y}; or
(ii) is selected from 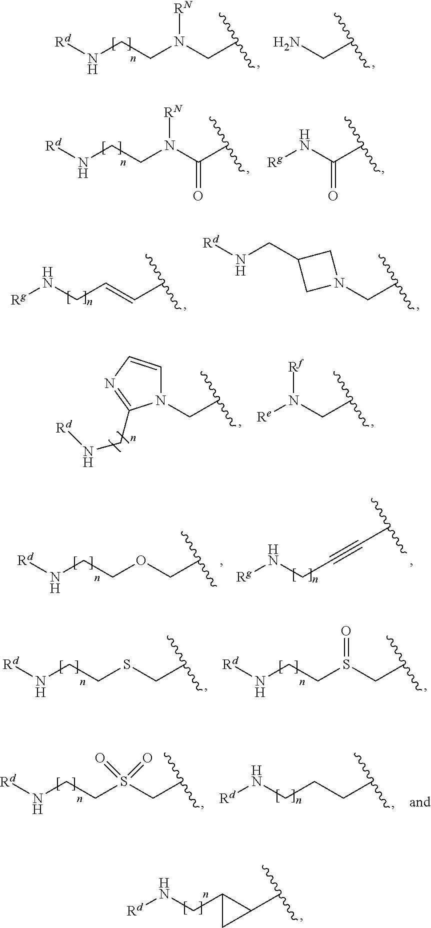
wherein
each substitutable carbon atom in the aforementioned groups is independently unsubstituted or substituted with one or more, same or different substituents selected from halogen, or two substituents together form C(═O);
R^{N} is H, C₁-C₄-alkyl, C₁-C₄-haloalkyl, NR^{a}R^{b}, cyclopropyl-C₁-C₄-alkyl, or a 5- or 6-membered saturated, partially or fully unsaturated or aromatic carbocyclyl, or heterocyclyl, wherein the aforementioned heterocyclic ring comprises one or more, same or different heteroatoms selected from O, N and S, wherein said N- and/or S-atoms are independently oxidized or non-oxidized and wherein each substitutable carbon or heteroatom in the aforementioned groups is independently unsubstituted or substituted with one or more, same or different substituents R^{X}; and
wherein
R^{a}, R^{b} are independently of each other selected from H, C₁-C₄-alkyl, and phenyl;
R^{c} is H, C₁-C₄-alkyl, NR^{a}R^{b}, NR^{a}R^{b}-C₁-C₄-alkyl, benzyl, or a 5- or 6-membered saturated heterocyclyl, wherein the aforementioned heterocyclic ring comprises one or more, same or different heteroatoms selected from O, N and S, and wherein said N- and/or S-atoms are independently oxidized or non-oxidized;
R^{d} is H, C(═O)R^{c}, phenyl-C₁-C₄-alkyl, benzophenone-C₁-C₄-alkyl, phenoxybenzene-C₁-C₄-alkyl, N-methyl-diphenylamine-C₁-C₄-alkyl, diphenylsulfide-C₁-C₄-alkyl, or naphthalene-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae 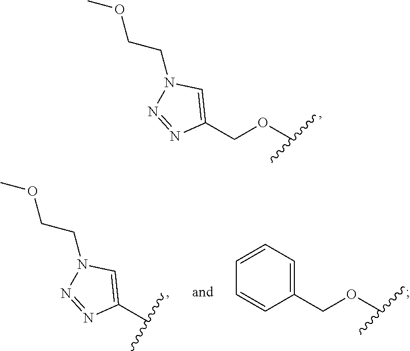
R^{e} is C₁-C₄-alkyl, C(═O)R^{c}, or a 5- or 6-membered saturated heterocyclyl, or heterocyclyl-C₁-C₄-alkyl, wherein said heterocyclic rings comprise one or more, same or different heteroatoms selected from O, N and S, wherein said N- and/or S-atoms are independently oxidized or non-oxidized, and wherein each substitutable carbon or heteroatom in the aforementioned groups is independently unsubstituted or substituted with one or more, same or different substituents R″;
R^{f} is H, or C₁-C₄-alkyl; or
R^{e} and R^{f} together with the nitrogen atom to which they are bonded form a 7-to 10-membered saturated spiro-heterocyclyl, wherein the aforementioned heterocyclic ring comprises one or more, same or different heteroatoms selected from O, N and S, and wherein said N- and/or S-atoms are independently oxidized or non-oxidized;
R^{g} is H, C(═O)R^{c}, phenyl-C₁-C₄-alkyl, benzophenone-C₁-C₄-alkyl, phenoxybenzene-C₁-C₄-alkyl, N-methyl-diphenylamine-C₁-C₄-alkyl, diphenylsulfide-C₁-C₄-alkyl, or naphthalene-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae 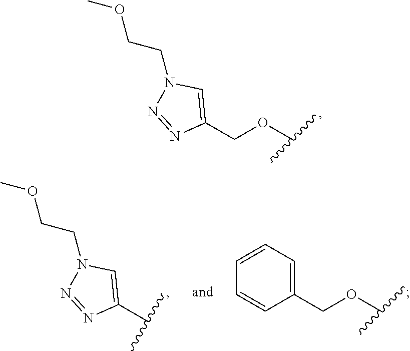
R^{h} is C₁-C₄-alkyl, or phenyl;
Rⁱ is C₁-C₄-alkyl, or phenyl;
R^{x} is halogen, C₁-C₄-alkyl, C(═O)R^{c}, S(═O)₂R^{h}, ORⁱ, or phenyl-C₁-C₄-alkyl, or two R^{x} form ═O;
R^{Y} is halogen, NH₂, OH, C₁-C₄-alkyl, C₁-C₄-haloalkyl, C₂-C₆-alkenyl, C₁-C₄-alkoxy, C₁-C₄-haloalkoxy, cyclopropyl, cyclopropyl-C₁-C₄-alkyl, phenyl-C₁-C₄-alkyl, two R^{Y} form-O, or a 4-to 6-membered saturated, partially or fully unsaturated or aromatic carbocyclyl, carbocyclyl-C₁-C₄-alkyl, heterocyclyl or heterocyclyl-C₁-C₄-alkyl, wherein the aforementioned heterocyclic rings comprise one or more, same or different heteroatoms selected from O, N and S, wherein said N- and/or S-atoms are independently oxidized or non-oxidized, and wherein each substitutable carbon in the aforementioned groups is independently unsubstituted or substituted with one or more, same or different substituents selected from NH₂, or two substituents form ═O; and
n is 1, 2, 3 or 4.

11. The method according to claim 10, wherein said prostate cancer is castration resistant prostate cancer.

12. The method according to claim 10, wherein in said compound of formula (I)
R^{x} is halogen, C₁-C₄-alkyl, C(═O)R^{c}, or phenyl-C₁-C₄-alkyl, or two R^{x} form ═O.

13. The method according to claim 10, wherein in said compound of formula (I)
X¹ is CH₂, or O;
X² is CR⁴;
X³ is N; and
X⁴ is N.

14. The method according to claim 10, wherein in said compound of formula (I)
R¹, R² are OH;
R³ is H; and
R⁶ is H, halogen, or C₁-C₄-alkyl.

15. The method according to claim 10, wherein in said compound of formula (I)
R⁴ is H, halogen, C₁-C₄-alkyl, C₂-C₄-alkenyl, phenyl, or a 3-to 6-membered fully unsaturated or aromatic heterocyclyl, wherein the aforementioned heterocyclic ring comprises one or more, same or different heteroatoms selected from O, N and S, wherein said N- and/or S-atoms are independently oxidized or non-oxidized, and wherein each substitutable carbon or heteroatom in the aforementioned groups is independently unsubstituted or substituted with one or more same or different substituents R^{x};
R⁵ is H, C₁-C₄-alkyl, or NR^{a}R^{b}; or
R⁴ and R⁵ together with the atoms to which they are bonded form a 7-to 10-membered partially or fully unsaturated carbocyclyl or heterocyclyl, wherein the aforementioned heterocyclic ring comprises one or more, same or different heteroatoms selected from O, N and S, wherein said N- and/or S-atoms are independently oxidized or non-oxidized, and wherein each substitutable carbon or heteroatom in the aforementioned groups is independently unsubstituted or substituted with one or more, same or different substituents R^{x}.

16. The method according to claim 10, wherein in said compound of formula (I)
L (i) is a 4-to 6-membered saturated, partially or fully unsaturated or aromatic heterocyclyl, or heterocyclyl-C₁-C₄-alkyl, wherein the aforementioned heterocyclic rings comprise one or more, same or different heteroatoms selected from O, N and S, and wherein said N- and/or S-atoms are independently oxidized or non-oxidized, and wherein each substitutable carbon or heteroatom in the aforementioned groups is independently unsubstituted or substituted with one or more, same or different substituents R^{Y}, or
a 5- or 6-membered aromatic carbocyclyl, wherein the aforementioned carbocyclic ring is independently unsubstituted or substituted with one or more, same or different substituents R^{Y}; or
(ii) is selected from 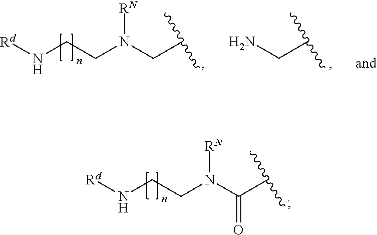
wherein
R^{N} is H, or a 5- or 6-membered saturated, partially or fully unsaturated or aromatic carbocyclyl, or heterocyclyl, wherein the aforementioned heterocyclic ring comprises one or more, same or different heteroatoms selected from O, N and S, wherein said N- and/or S-atoms are independently oxidized or non-oxidized and wherein each substitutable carbon or heteroatom in the aforementioned groups is independently unsubstituted or substituted with one or more, same or different substituents R^{x}; and
wherein
R^{d} is H, phenyl-C₁-C₄-alkyl, benzophenone-C₁-C₄-alkyl, phenoxybenzene-C₁-C₄-alkyl, or naphthalene-C₁-C₄-alkyl, wherein each substitutable carbon or heteroatom in the aforementioned groups is independently unsubstituted or substituted with one or more, same or different substituents selected from halogen, C₁-C₄-haloalkyl, and phenyl-C₁-C₄-alkyl, or a substituent according to the following formulae

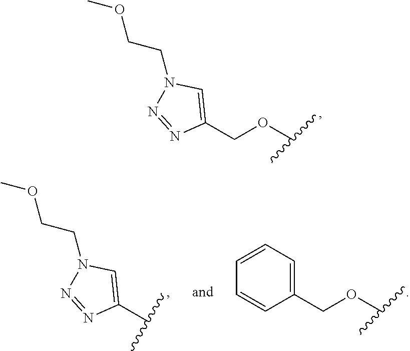

17. A method of treating castration resistant prostate cancer, the method comprising administering a pharmaceutical composition comprising a pharmaceutically effective amount of the compound of formula (I) as defined in claim 10 and a pharmaceutically acceptable carrier, diluent, or excipient.
